(19) United States
(12) Patent Application Publication
(10) Pub. No.: US 2023/0303666 A1
(43) Pub. Date: Sep. 28, 2023

(54) CELL-FREE ANTIBODY ENGINEERING PLATFORM AND NEUTRALIZING ANTIBODIES FOR SARS-COV-2

(71) Applicants: The Broad Institute, Inc., Cambridge, MA (US); Massachusetts Institute of Technology, Cambridge, MA (US)

(72) Inventors: Aviv Regev, Cambridge, MA (US); Xun Chen, Cambridge, MA (US)

(21) Appl. No.: 18/026,866

(22) PCT Filed: Sep. 24, 2021
(86) PCT No.: PCT/US21/51925

§ 371 Date: Mar. 17, 2023

Related U.S. Application Data
(60) Provisional application No. 63/083,073, filed on Sep. 24, 2020.
(60) Provisional application No. 63/221,663, filed on Jul. 14, 2021.

(51) Int. Cl. C07K 16/10; A61P 31/14; C12N 15/10

(52) U.S. Cl. CPC C07K 16/10; A61P 31/14; C12N 15/1093; C07K 2317/565; C07K 2317/569; C07K 2317/22; C07K 2317/24

(57) ABSTRACT

The present invention discloses antibodies capable of binding to and neutralizing SARS-CoV-2 and variants thereof. The invention also discloses a cell-free antibody engineering platform capable of identifying antibodies that bind to specific target molecules and virus-neutralizing antibodies.

### CROSS-REFERENCE TO RELATED APPLICATIONS

This application claims the benefit of U.S. Provisional Application Nos. 63/083,073, filed Sep. 24, 2020, and 63/221,663, filed Jul. 14, 2021. The entire contents of the above-identified applications are hereby fully incorporated herein by reference.

### STATEMENT REGARDING FEDERALLY SPONSORED RESEARCH

This invention was made with government support under Grant No. HG006193 awarded by the National Institutes of Health. The government has certain rights in the invention.

### REFERENCE TO AN ELECTRONIC SEQUENCE LISTING

The contents of the electronic sequence listing (“BROD-5260WP_ST25.txt”; Size is 4,302,794 bytes and it was created on Sep. 20, 2021) is herein incorporated by reference in its entirety.

### TECHNICAL FIELD

The subject matter disclosed herein is generally directed to an integrated platform for generating and engineering antibodies and its application in developing SARS-CoV-2 neutralizing antibodies.

### BACKGROUND

Antibodies and their functional domains play key roles in research, diagnostics and therapeutics. Antibodies are traditionally made by immunizing animals with the desired target as antigen, but such methods are time consuming, their outcome is often unpredictable, and their use is increasingly restricted in the European Union 1. Alternatively, antibodies can be generated and selected in vitro, where libraries of antibody-encoding DNA, either fully synthetic or derived from animals, are displayed in vitro followed by selection and recovery of those binding the intended target^{2,3}. However, the adoption of such in vitro methods is still more limited than that of animal-dependent antibody generation⁴, possibly due to throughput limitations and concerns over functional fitness and in vivo tolerance of antibodies generated in vitro 5. Recent advances in antibody library design and construction, in vitro display and selection methods, post-selection binder identification and maturation have helped increase the utility of in vitro antibody generation². For example, recently developed antibody library designs have been successfully used with in vitro display methods for engineering antibodies⁶⁻⁸.

For typical antibodies, antigen binding is co-determined by the variable domains of both its heavy chain (VH) and light chain (VL/VK), while camelids produce unconventional heavy-chain-only antibodies that bind to antigens solely based on the variable domain of their heavy chain, the VHH domain (also known as nanobodies). Nanobodies are increasingly used as functional antibody domains because of their small size (˜14 kD)⁹ and high stability (Tₘ, up to 90° C.)¹⁰. Nanobody libraries have been successfully screened for binders by phage and yeast display^{6,11,12}. However, the screening diversity of such cell-based systems has often been limited in practice by the efficiency of DNA library delivery into cells (e.g., transformation efficiency of E. coli is typically <10¹⁰). Conversely, cell-free approaches, such as ribosome display¹³, are not limited by cell transformation and culture constraints. Despite these potential advantages, ribosome display remains underutilized compared to cell-based display systems², possibly due to sub-optimal efficiency and fidelity of cell free reactions. Further optimization can open up this methodology for antibody screening and enable wider adoption of cell-free systems in antibody engineering.

Severe acute respiratory syndrome coronavirus 2 (SARS-CoV-2) is the virus causing the ongoing Coronavirus Disease 19 (COVID19) pandemic. Identifying neutralizing antibodies is important for the development of effective therapeutics.

Citation or identification of any document in this application is not an admission that such a document is available as prior art to the present invention.

### SUMMARY

In one aspect, the present invention provides for an antibody or antigen binding fragment comprising one or more complementarity-determining regions (CDRs) selected or derived from any cluster or CDRs in any of Tables 1-9 (SEQ ID NOS: 1-5872). In certain embodiments, the CDRs are selected or derived from the SR1, SR2, SR4, SR6, SR8, SR12, SR15, SR18, SR25, SR30 or SR38 cluster families. In certain embodiments, the antibody or antigen binding fragment comprises CDRs from SR6v15, SR6v7, SR38, SR6c3, SR4t13, or SR2c3. In certain embodiments, the antibody or antigen binding fragment is a heavy chain antibody or variable domain of the heavy chain (VHH). In certain embodiments, the heavy chain antibody or variable domain of the heavy chain (VHH) is SR38 and binds to a N501Y SARS-CoV-2 variant. In certain embodiments, the heavy chain antibody or variable domain of the heavy chain (VHH) is SR6v15. In certain embodiments, the heavy chain antibody or variable domain of the heavy chain (VHH) is a dimer of SR6v15. In certain embodiments, the heavy chain antibody or variable domain of the heavy chain (VHH) is SR6v7. In certain embodiments, the heavy chain antibody or VHH are derived from camelid heavy chain antibodies. In certain embodiments, one or more framework residues in camelid antibodies are humanized. In certain embodiments, the humanized residues are located in one or more positions selected from the group consisting of frame 2 position 4, frame 2 position 11, frame 2 position 12, frame 2 position 14, and frame 4 position 8. In certain embodiments, the antibody or antigen binding fragment is modified to alter binding affinity, stability, in vivo half-life, neutralizing activity and/or dimerization. In certain embodiments, the antibody or antigen binding fragment is a fusion protein. In certain embodiments, the antibody or antigen binding fragment is fused to another antibody or antibody fragment, Fc domain, antigen binding domain, glutathione S-transferase (GST), and/or serum albumin.

In another aspect, the present invention provides for a method of treating SARS-CoV-2 infection comprising administering to a subject in need thereof the antibody or antigen binding fragment of any embodiment herein. In certain embodiments, the subject is infected with a SARS-CoV-2 variant. In certain embodiments, SR38 is administered to the subject. In certain embodiments, the subject is infected with a SARS-CoV-2 variant containing the N501Y mutation. In certain embodiments, SR6v15 is administered to the subject. In certain embodiments, a dimer of SR6v15 is administered to the subject.

In another aspect, the present invention provides for a method of detecting SARS-CoV-2 comprising contacting a biological sample obtained from a subject with the antibody or antigen binding fragment of any embodiment herein. In certain embodiments, the antibody is SR38 and a N501Y variant is detected. In certain embodiments, the antibody is SR38 and a E484K variant is detected. In certain embodiments, the antibody is SR6v15.

In another aspect, the present invention provides for a method of generating a VHH library comprising a VHH template with a randomized CDR1, CDR2 and CDR3 comprising: a. providing a VHH template; b. providing a first set of primers capable of amplifying the VHH template from a first CDR sequence to the end of the template, wherein the set of primers comprise: i. a primer comprising a 5′ randomized sequence corresponding to all or part of the first CDR sequence and a 3′ sequence capable of hybridizing to a non-randomized sequence; and ii. a hairpin primer capable of hybridizing to one end of the template; c. providing a second set of primers capable of amplifying the VHH template from the sequence directly adjacent to where the first primer set amplified from to the other end of the template, wherein the set of primers comprise: i. a primer capable of hybridizing to the sequence directly adjacent to where the first primer set amplified from, optionally, wherein the primer starts within the first CDR sequence and comprises a 5′ randomized sequence corresponding to the remaining first CDR sequence and a 3′ sequence capable of hybridizing to a non-randomized sequence; and ii. a hairpin primer capable of hybridizing to the other end of the template; d. PCR amplifying the VHH template with the first and second sets of primers to generate two single-end blocked PCR products corresponding to the entire VHH template; e. ligating the two PCR products; f repeating steps (a) to (e) for the second CDR sequence, wherein the randomized VHH ligation product obtained in step (e) is used as the template, whereby a VHH template randomized for two CDRs is obtained; and g. repeating steps (a) to (e) for the third CDR sequence, wherein the randomized VHH ligation product obtained in step (f) is used as the template, whereby a VHH template randomized for all three CDRs is obtained. In certain embodiments, the primer sequences are 5′ NNB randomized, where N is a mixture of A, T, G, C bases, and B is a mixture of G, C, T bases. In certain embodiments, the primer sequences are 5′ randomized using NNN tri-nucleotide sequence, where N is a mixture of A, T, C, G nucleotides. In certain embodiments, step (d) is performed using a DNA polymerase without strand displacement activity or with weak strand displacement activity. In certain embodiments, step (d) is performed using an elongation temperature of 65° C. In certain embodiments, CDR2 is randomized first. In certain embodiments, CDR1 is randomized second. In certain embodiments, CDR3 is randomized last. In certain embodiments, CDR2 encodes for 4 or 5 amino acids. In certain embodiments, CDR1 encodes for 4 to 8 amino acids. In certain embodiments, CDR3 encodes for 4 to 30 amino acids. In certain embodiments, the VHH templates comprise a promoter sequence upstream of the VHH template. In certain embodiments, the promoter is a T7 promoter. In certain embodiments, the VHH templates comprise an epitope tag sequence downstream of and in frame with the VHH template. In certain embodiments, the epitope tag comprises one or more myc tags. In certain embodiments, the method further comprises displaying the CDR1 and/or CDR2 randomized library of step (e) or (f) with ribosome display; enriching library members using the epitope tag; and using the enriched DNAs for input in step (g). In certain embodiments, the VHH templates do not include a stop codon.

In another aspect, the present invention provides for a method of identifying CDRs for generating an antibody or binding fragment of an antibody specific to an antigen of interest comprising: a. providing a linear DNA library, wherein each sequence in the library encodes for an antibody framework comprising three CDRs and operably linked to a 5′ promoter sequence, and wherein at least one CDR is randomized; b. performing ribosome display on the linear DNA library, whereby mRNAs transcribed from the linear DNA library are translated to an antibody protein that is tethered to the ribosome ribonucleoprotein complex; c. binding the ribonucleoprotein complexes to an immobilized antigen of interest; d. performing reverse transcription PCR (RT-PCR) on mRNA extracted from ribonucleoprotein complexes bound to the immobilized antigen, whereby cDNA is generated; e. optionally, repeating steps (b) to (d) using the cDNA from the bound ribonucleoprotein complexes as the linear DNA input; f sequencing the cDNAs to obtain antibody sequences; and g. clustering the antibody sequences based on similarity of their CDRs to identify distinct antibody clusters containing CDRs specific to the antigen of interest. In certain embodiments, all three CDRs are randomized. In certain embodiments, the CDRs are encoded by DNA oligos with 5′ NNB or NNN randomized sequences, where N is a mixture of A, T, G, C bases, and B is a mixture of G, C, T bases. In certain embodiments, step (c) is performed in solutions containing Mg2+ ions at concentrations of 5 mM or less. In certain embodiments, step (d) is performed using a mixture of two DNA polymerases in the PCR reaction, wherein one type is a DNA polymerase without strand displacement activity or with weak strand displacement activity and the other type is a DNA polymerase with strong strand displacement activity. In certain embodiments, steps (b) to (d) are performed for three rounds. In certain embodiments, the method further comprises identifying amino acid substitutions that will increase antibody binding and/or viral neutralization activity, said method comprising: h. introducing random mutations across the full length of one or more identified antibody frameworks using error prone PCR to obtain a mutated linear DNA library; i. repeating steps (b) to (d) for 1 to 3 rounds using the linear DNA library obtained in step (h) as the linear DNA library; j. sequencing the linear DNA library in (h) and the cDNA obtained in (i); k. calculating the percentage of each proteinogenic amino acid found at each antibody framework amino acid position among all sequenced antibody frameworks obtained from (h) and (i); 1. identifying amino acids at each position with increased percentage in (i) as compared to in sequences from (h); and m. replacing the amino acids at said positions in the antibody framework with the identified amino acids. In certain embodiments, step (c) is performed using a binding time of less than 1 minute. In certain embodiments, CDRs are selected from one or more of the clusters having the largest number of members. In certain embodiments, the antibody frameworks are heavy chain antibody variable domains (VHHs). In certain embodiments, the VHHs are camelid VHHs. In certain embodiments, the linear DNA library in step (a) is obtained according to the method of any embodiment herein. In certain embodiments, the method further comprises validating at least one member of a cluster or VHH sequence with amino acid substitutions by expressing the antibody framework and determining binding to the antigen of interest. In certain embodiments, the antigen of interest is associated with a viral pathogen and the antibody framework is tested for neutralizing activity. In certain embodiments, the method further comprises transferring one or more of the CDRs to a different antibody framework. In certain embodiments, the method further comprises synthesizing one or more sequences from each antibody cluster for cloning of the antibody genes and testing the antibody proteins.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1J-A cell-free nanobody engineering platform for rapid isolation of nanobodies from large synthetic libraries. (FIG. 1a) The workflow takes linear DNA library as input. (FIG. 1B) Ribosome display links genotype (RNAs transcribed from DNA input library that are stop codon free, and stall ribosome at the end of the transcript) and phenotype (folded VHH protein tethered to ribosomes due to the lack of stop codon in the RNA). (FIG. 1c) Selection cycle that enriches DNA encoding for VHHs that binds immobilized targets. (FIG. 1d) High throughput sequencing of full-length VHHs. (FIG. 1e) Sequences are grouped into clusters based on similarity of their CDRs, each cluster is distinct and represent a unique binding family. (FIG. 1f) The system outputs one representative sequence from each cluster to be synthesized and characterized for specific downstream applications. (FIG. 1g) Workflow for generating VHH library. VHH CDR randomization was introduced by PCR using a hairpin oligo (blocks DNA end from ligation) and an oligo with random 5′ sequence, followed by orientation-controlled ligation. Three successive PCR plus ligation cycles randomizes all three CDRs. (FIG. 1h) The final DNA library sequence structure. (FIG. 1i) One round of ribosome display and anti-Myc selection was performed after randomization of CDR1 and CDR2. The pie chart shows percentage of indicated sequence categories before and after anti-Myc selection. (FIG. 1j) Length distribution of DNA region encoding CDR1 of the VHH library before and after anti-Myc selection. Arrows indicate all correct-frame lengths showing increased percentage after anti-Myc selection.

FIGS. 2A-2D—Amino acid profiles of natural and synthetic VHHs (nanobodies). (FIG. 2a) Position-wise amino acid profile of natural VHHs (298 VHHs, PDB) and (FIG. 2b) synthetic VHHs. Amino acids were color coded according to labels to the right, B indicates an empty position. Bar height is the relative percentage of each amino acids. The two most common amino acids were shown as patterned bars while others were shown as solid bars. (FIG. 2c) Plot of diversity index (as 1—Gini index) for each amino acid position of natural VHHs and (FIG. 2d) synthetic VHHs.

FIGS. 3A-3C—Design of VHH frames and their homology to human IGH genes. (FIG. 3a) Amino acid sequences encoded by frames that serve as templates for VHH library generation were aligned to the corresponding segments of the human IGHV3-23 (hIGHV3-23) or IGHJ4 (hIGHJ4). Positions in hIGHV3-23/hIGHJ4 that are identical to the corresponding position in at least one VHH frames are highlighted in orange. Positions in VHH frames that are identical to the corresponding position in hIGHV3-23/hIGHJ4 are highlighted in orange. hIGHV3-23 positions not identical to any VHH frames are numbered according to its position within the segment. Asterisks indicate VHH hallmark residues thought to be required for VHH's independence of light chain. (FIG. 3b) Percent homology of VHH frames to the closest human gene. (FIG. 3c) List of VHH residues at positions numbered in (a) and representative human IGHV genes that encode the same VHH residue at the corresponding position. None: no human IGHV genes has the VHH residue at the corresponding position.

FIGS. 4A-4B—Working principles of orientation-controlled ligation by end blocking using hairpin oligos. (FIG. 4a) working principle for generating one end blocked DNA for orientation-controlled ligation by PCR using a hairpin DNA oligo. (FIG. 4b) Representative orientation-controlled ligation products visualized by agarose gel electrophoresis.

FIGS. 5—Comparison of the CDR2 region in the CeVICA nanobody library and previous designs. Alignment of CDR2 and neighboring sequences designed for four libraries (rows). X: highly diversified positions (>=10 different amino acids); blue triangles: positions with limited diversity (<10 different amino acids).

FIGS. 6A-6H—Isolation and characterization of synthetic VHHs that binds SARS-CoV-2 spike RBD. (FIG. 6a) Immobilization strategy for the target proteins: 3×Flag-tagged EGFP or RBD. (FIG. 6b) Pair-wise CDR match score (based on BLOSUM62 matrix) were calculated for 2000 randomly selected sequences from input library and output libraries after 3 rounds of selection. High match score populations appeared in the output libraries. Combining CDR1 and 2 match scores further separated high and low score population and a match score of 35 (black dashed line) was chosen as cut-off for downstream clustering analysis. (FIG. 6c) Percentage of indicated sequence categories in the input library and output libraries (EGFP, RBD). (FIG. 6d) Number of unique and shared clusters identified in EGFP and RBD output libraries. (FIG. 6e) Number of sequences for each size of RBD unique clusters. (FIG. 6f) ELISA assay revealed 3 strong binders (“s”) to RBD, 8 weak binders (“w”) and (FIG. 6g) 3 non-binders (“n”, background subtracted OD 450 nm<0.02) among the 14 VHHs chosen for characterization. (FIG. 6h) SARS-CoV-2 S pseudotyped lentivirus neutralization assay showed 6 VHHs inhibiting infection >30% at 1 μM on HEK293T expressing ACE2 and TMPRSS2. Data shown are two technical replicates, bars indicate the average of data, circles indicate values of each replicate.

FIGS. 7A-7F—Evaluation of ribosome display and selection rounds. (FIG. 7a) Yield of recovered RNA at each round of ribosome display and selection for EGFP or RBD targets. (FIG. 7b) Representative RT reaction (without heat denaturation) product for RBD selection after 3 rounds, visualized by agarose gel electrophoresis. (FIG. 7c) Plot of match scores of sequence pairs with a combined CDR1 and CDR2 score >35. (FIG. 7d) Plot of match scores of sequence pairs (from 2000 randomly sampled sequences) with indicated CDR1 scores, and (FIG. 7e) indicated CDR2 scores, and (FIG. 70 indicated CDR3 scores.

FIGS. 8A-8E—Unique output binders amino acid profiles is more similar to that of input library than natural VHHs. (FIG. 8a) Spearman correlation coefficient values for the amino acid percentages in the indicated sequence group pairs at each CDR position. 298 natural nanobodies (natural) and 298 randomly sampled sequences from input library (input) and output binders (output) were analyzed. Three random sampling trials were performed to generate three Spearman correlation coefficient for each position. **: p<0.01, *: p<0.05 (t test between output vs. input and output vs. natural values). (FIG. 8b) Scatter plots of the percentage of each amino acid in the input library and the output binders and (FIG. 8c) that in the natural nanobodies and the output binders at representative CDR positions. A few data points are out of the range of the set axes due to extreme “outlier” values in the natural profile, see Table 11 for all data point values. Circle: mean, error bar: standard deviation. (FIG. 8d) Root mean square error (RMSE, relative to y=x line) values for the indicated sequence group pairs at each CDR position. Using the same randomly sampled sequences as (a). **: p<0.01, *: p<0.05 (t test between output vs. input and output vs. natural values). (FIG. 8e) Three-way distance maps of the distances between the three groups, with the length of each line connecting between two sequence groups indicating their RMSE. The input group (input) is fixed at (0,0), the natural group (natural) is fixed on the x axis (0,y), and the position of output group (output) is calculated based on its distance (RMSE) to the input and natural groups. Vertical dashed lines indicate the middle point of the distance between the input and natural groups.

FIGS. 9A-9B—Amino acid profile for EGFP and RBD unique output binders. (FIG. 9a) Amino acid profile of representative VHH sequence for each unique cluster identified from RBD and EGFP output libraries (“output binders”, 932 sequences). Plotted as described in FIG. 2a. (FIG. 9b) Plot of diversity index (as 1—Gini index) for each amino acid position of output binder VHHs.

FIG. 10A-10B—Comparison of CeVICA input and output amino acid profiles to that of 1,030 natural nanobody sequences in the abYsis collection. (FIG. 10a) Root mean square error (RMSE, relative to y=x line) values for the amino acid percentages in 1,030 nanobodies from abYsis (natural), and 350 randomly sampled sequences from either the input (input) or output binders (output) libraries. Three random sampling trials were performed to generate three RMSE for each position. **: p<0.01, t test of the difference between output vs. input and output vs. natural. (FIG. 10b) Three-way distance maps of the distances between the three groups, with the length of each line connecting between two sequence groups indicating their RMSE. The input group (input) is fixed at (0,0), the natural group (natural) is fixed on the x axis (0,y), and the position of output group (output) is calculated based on its distance (RMSE) to the input and natural groups. Vertical dashed lines indicate the middle point of the distance between the input and natural groups.

FIG. 11A-11G—An affinity maturation strategy enhances binding and neutralization properties of synthetic VHHs. (FIG. 11a) Affinity maturation workflow. (FIG. 11b) Two representative sections of position-wise post-minus pre-affinity maturation amino acid percent point change profile. White values indicate the original amino acid, yellow values indicate the beneficial mutation. Empty positions indicate amino acids not detected in either the pre- or post-selection libraries. (FIG. 11c) ELISA assay of VHH variants. (FIG. 1id) SARS-CoV-2 S pseudotyped lentivirus neutralization assay of VHHs on HEK293T expressing ACE2 and TMPRSS2. For (c) and (d), data shown are two technical replicates, bars indicate the average of data, circles indicate values of each replicate. (FIG. 11e) Scatter plot of ELISA assay absorbance versus pseudotyped lentivirus neutralization as percent infection inhibited. VHH concentration for both assays were 50 nM. Values are average of two technical replicates. Numbers on linear fitting lines were r² value for data within each family. (FIG. 111) Dose-response curve for neutralization of pseudotyped lentiviral infection by VHHs. Markers are average of three technical replicates, error bars are standard deviation. (FIG. 11g) IC50 calculated from data in (f), presented as mean±standard deviation.

FIG. 12—Affinity maturation leads to some VHH hallmark residues converting to the corresponding human VII residues. The post-minus pre-affinity maturation percent point change of VHH hallmark residues and the corresponding human residues for each VHH. Arrows indicate human residues with increased frequency as a result of affinity maturation.

FIG. 13A-13C—Identification of additional RBD binders and pseudovirus neutralizers among lower ranking clusters. (FIG. 13a, FIG. 13b) Binding assay. Binding measured by ELISA assay (y axis, OD 450 nm) of each of the nanobodies (x axis) tested at 1 for binding to wild type RBD (RBD wt) or RBD carrying the N501Y (RBD N501Y) or E484K (RBD E484K) mutations. Non binder: 5 nanobodies that showed undetectable values (background subtracted OD 450 nm<0.02). (FIG. 13c) Pseudovirus neutralization. SARS-CoV-2 S pseudotyped lentivirus neutralization of nanobodies at 1 μM on HEK293T expressing ACE2 and TMPRSS2. Data shown are two technical replicates, bar height: mean, circle: value of each replicate.

FIG. 14A-14D-A second affinity maturation generates neutralizing agents with picomolar IC50. (FIG. 14a) Binding (y axis, ELISA assay) of new SR6 variants identified by the second affinity maturation and two previously reported nanobodies, Nb21 and Ty1 (x axis). Nanobody concentrations shown at the bottom. Data shown are two technical replicates, bar height: mean, circle: value of each replicate. (FIG. 14b) Biolayer interferometry assay of SR6v15. Red traces: recorded sensorgrams, black traces: fitted curves. K_{D}, Kₐ and K_{d} values are mean of five measurements. (FIG. 14c) Pseudovirus neutralization. % inhibition (y axis) of different nanobodies (x axis). Data shown are two technical replicates, bar height: mean, circle: value of each replicate. (FIG. 14d) Dose-response curve for neutralization of pseudotyped lentiviral infection by nanobodies and nanobody-based agents. Markers: mean of three technical replicates, error bars: standard deviation. IC50 values are shown as mean±standard deviation.

FIG. 15A-15C—Representative nanobodies mainly exist as monomers in solution. Size-exclusion chromatography traces of SR12 (FIG. 15a), SR18 (FIG. 15b), and SR6c3 (FIG. 15c). Percent monomer values are shown next to the monomer peaks.

FIG. 16A-16D—Intramolecular disulfide bond formation via CDR cysteine does not impair SR6c3 function. (FIG. 16a, FIG. 16b) Coomassie blue stained SDS PAGE gel of nanobody samples prepared in (a) non-reducing sample buffer or (b) in non-reducing (−) or reducing (+) sample buffer. Number of months: length of time the sample has been stored at 4° C. (FIG. 16c) Binding (y axis, by ELISA) of SR6c3 samples stored for different durations with or without treatment with DTT (50 mM DTT at room temperature for 2 hours). (FIG. 16d) SARS-CoV-2 S pseudotyped lentivirus neutralization of SR6c3 samples on HEK293T expressing ACE2 and TMPRSS2. Data shown are two technical replicates, bar height: mean, circle: value of each replicate.

FIG. 17A-17B—Thermal stability and refolding analysis of nanobodies. (FIG. 17a) Protein thermal shift assay. Melt curve derivative (y axis) at different temperatures (x axis) for two nanobodies. (FIG. 17b) Refolding assay following thermal denaturation. Ratio of binding (ELISA OD 450 nm) of heated (98° C. for 10 minutes) vs. non-heated samples (y axis) for different nanobodies (x axis). DTT: 25 mM DTT in sample solution. Data shown are three technical replicates, bar height: mean, circle: value of each replicate.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Embodiments disclosed herein provide a cell-free antibody engineering platform for rapid isolation of antibodies from synthetic library and antibodies obtained by the platform. Antibody engineering technologies face increasing demands for speed, reliability and scale. Applicants developed CeVICA, a cell-free antibody engineering platform that uses ribosome display for in vitro selection of nanobodies from a library of 1011 randomized sequences. Applicants applied CeVICA to engineer antibodies against the Receptor Binding Domain (RBD) of SARS-CoV-2 spike proteins and identified >800 binder families using a computational pipeline based on CDR-directed clustering. Among 38 experimentally tested families, 30 were true RBD binders and 11 inhibited SARS-CoV-2 pseudotyped virus infection. Affinity maturation and multivalency engineering increased nanobody binding affinity and yielded a virus neutralizer with picomolar IC50. Furthermore, the unique capability of CeVICA for comprehensive binder prediction allowed retrospective validation of the synthetic VHH library fitness. CeVICA offers an integrated solution to rapid generation of divergent synthetic antibodies with tunable affinities in vitro and may serve as the basis for automated and highly parallel antibody generation. The identified antibodies can be used for treatment of SARS-CoV-2 and variants thereof. The identified antibodies can be used for detection of SARS-CoV-2 and variants thereof.

### Therapeutic Antibodies or Binding Fragments of an Antibody

### Antibodies

In certain embodiments, the present invention provides antibodies, antibody fragments, binding fragments of an antibody, or antigen binding fragments capable of binding to an antigen of interest (e.g., the receptor binding domain of SARS-CoV-2). The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

In certain embodiments, the antibody or antibody fragment is a therapeutic antibody. In certain embodiments, the antibody is a neutralizing antibody. As used herein, “neutralizing antibody” refers to an antibody that is capable of neutralizing a pathogen or reducing infectivity, such as a viral pathogen (e.g., SARS-CoV-2). In certain embodiments, the antibody or antibody fragment is capable of use in detection (e.g., SARS-CoV-2).

Applicants have used the methods described further herein to identify specific antibodies capable of neutralizing SARS-CoV-2. The antibodies specifically bind to the receptor binding domain of SARS-CoV-2 spike and neutralize pseudotyped virus expressing the spike protein.

The antibodies or antibody fragments disclosed herein can also bind to and/or neutralize SARS-CoV-2 variants. As used herein, the term “variant” refers to any virus having one or more mutations as compared to a known virus. A strain is a genetic variant or subtype of a virus. The terms ‘strain’, ‘variant’, and ‘isolate’ may be used interchangeably. In certain embodiments, a variant has developed a “specific group of mutations” that causes the variant to behave differently than that of the strain it originated from. While there are many thousands of variants of SARS-CoV-2, (Koyama, Takahiko Koyama; Platt, Daniela; Parida, Laxmi (June 2020). “Variant analysis of SARS-CoV-2 genomes”. Bulletin of the World Health Organization. 98: 495-504) there are also much larger groupings called clades. Several different Glade nomenclatures for SARS-CoV-2 have been proposed. As of December 2020, GISAID, referring to SARS-CoV-2 as hCoV-19 identified seven clades (0, S, L, V, G, GH, and GR) (Alm E, Broberg E K, Connor T, et al. Geographical and temporal distribution of SARS-CoV-2 clades in the WHO European Region, January to June 2020 [published correction appears in Euro Surveill. 2020 August; 25(33):]. Euro Surveill. 2020; 25(32):2001410). Also as of December 2020, Nextstrain identified five (19A, 19B, 20A, 20B, and 20C) (Cited in Alm et al. 2020). Guan et al. identified five global clades (G614, S84, V251, 1378 and D392) (Guan Q, Sadykov M, Mfarrej S, et al. A genetic barcode of SARS-CoV-2 for monitoring global distribution of different clades during the COVID-19 pandemic. Int J Infect Dis. 2020; 100:216-223). Rambaut et al. proposed the term “lineage” in a 2020 article in Nature Microbiology; as of December 2020, there have been five major lineages (A, B, B.1, B.1.1, and B.1.777) identified (Rambaut, A.; Holmes, E. C.; O'Toole, A.; et al. “A dynamic nomenclature proposal for SARS-CoV-2 lineages to assist genomic epidemiology”. 5: 1403-1407).

Genetic variants of SARS-CoV-2 have been emerging and circulating around the world throughout the COVID-19 pandemic (see, e.g., The US Centers for Disease Control and Prevention; www.cdc.gov/coronavirus/2019-ncov/variants/variant-info.html). Exemplary, non-limiting variants applicable to the present invention include variants of SARS-CoV-2 having substitutions of therapeutic concern (Table A).

TABLE A

| Spike Protein | Common Pango Lineages with Spike Protein |
|---|---|
| Substitution | Substitutions |
| L452R | A.2.5, B.1, B.1.429, B.1.427, B.1.617.1, |
| | B.1.526.1, B.1.617.2, C.36.3 |
| E484K | B.1.1.318, B.1.1.7, B.1.351, B.1.525, B.1.526, |
| | B.1.621, B.1.623, P.1, P.1.1, P.1.2, R.1 |
| K417N, E484K, N501Y | B.1.351, B.1.351.3 |
| K417T, E484K, N501Y | P.1, P.1.1, P.1.2 |

Phylogenetic Assignment of Named Global Outbreak (PANGO) Lineages is software tool developed by members of the Rambaut Lab. The associated web application was developed by the Centre for Genomic Pathogen Surveillance in South Cambridgeshire and is intended to implement the dynamic nomenclature of SARS-CoV-2 lineages, known as the PANGO nomenclature.

The SARS-CoV-2 variants applicable to the present invention include: B.1.1.7, also known as Alpha (WHO) or UK variant, having the following spike protein substitutions: 69de1, 70de1, 144de1, (E484K*), (S494P*), N501Y, A570D, D614G, P681H, T716I, S982A, and D1118H (K1191N*); B.1.351, also known as Beta (WHO) or South Africa variant, having the following spike protein substitutions: D80A, D215G, 241de1, 242de1, 243de1, K417N, E484K, N501Y, D614G, and A701V; B.1.427, also known as Epsilon (WHO) or US California variant, having the following spike protein substitutions: L452R, and D614G; B.1.429, also known as Epsilon (WHO) or US California variant, having the following spike protein substitutions: S13I, W152C, L452R, and D614G; B.1.617.2, also known as Delta (WHO) or India variant, having the following spike protein substitutions: T19R, (G142D), 156de1, 157de1, R158G, L452R, T478K, D614G, P681R, and D950N; and P.1, also known as Gamma (WHO) or Japan/Brazil variant, having the following spike protein substitutions: L18F, T20N, P26S, D138Y, R190S, K417T, E484K, N501Y, D614G, H655Y, and T10271.

The antibodies include “complementarity determining regions” or “CDRs” interspersed among “frame regions” or “FRs”, as defined herein. As used herein CDRs refer to variable regions in an antibody that provide for antigen specificity. In certain embodiments, specific CDRs identified can be used in an antibody framework described further herein. In certain embodiments, one, two, or all three CDRs are used in a framework. In certain embodiments, CDR1 and CDR3 are used in a framework. In certain embodiments, CDR3 is used in a framework. In preferred embodiments, all three CDRs are used in a framework. In certain embodiments, the CDRs are used in a heavy-chain antibody VHH domain. As used herein, framework can refer to an entire antibody VHH domain or antibody as described herein. In certain embodiments, frame region (FR) refers to the non-CDR regions or constant regions in the antibody. The frame regions in the antibodies of the present invention are also referred to as frame 1, frame2, frame3 and frame4.

In certain embodiments, the antibodies of the present invention are heavy chain antibodies. As used herein “heavy chain antibody,” “VHH” or “single-domain antibodies” (sdAbs) refers to an antibody which consists only of two heavy chains and lacks the two light chains usually found in antibodies (see, e.g., Henry and MacKenzie, Antigen recognition by single-domain antibodies: structural latitudes and constraints. MAbs. 2018 August-September; 10(6): 815-826). VHH can refer to an antibody or VHH domain. Single-domain antibodies (sdAb) are also known as a nanobody; an antibody fragment consisting of a single monomeric variable antibody domain. As used herein “VHH” is used interchangeably with “nanobody.” The ˜12-15 kDa variable domains of these antibodies (VHHs and VNARs) can be produced recombinantly and can recognize antigen in the absence of the remainder of the antibody heavy chain. In common antibodies, the antigen binding region consists of the variable domains of the heavy and light chains (VH and VL). Heavy-chain antibodies can bind antigens despite having only VH domains. In certain embodiments, the heavy chain antibody is an antibody derived from cartilaginous fishes (immunoglobulin new antigen receptor (IgNAR)) or camelid ungulates. Non-limiting examples of camelids include dromedaries, camels, llamas and alpacas.

Applicants specifically identified CDR clusters in camelid heavy chain antibody domains (VHH) that specifically bind to the receptor binding domain of SARS-CoV-2 spike and neutralize pseudotyped virus expressing the spike protein or that specifically bind to EGFP (Tables 1-9). Antibodies belonging to 6 clusters or families having similar CDRs were identified that have binding and neutralizing activity (Tables 1-7 and 9).

TABLE 1
Sequences belonging to cluster SR1.
Each line in the table represents one sequence,
both segments and full length of the sequence
are shown. Shown items are divided by “#” and
in the order from start to end of each line is:
CDR1 amino acid sequence, CDR2 amino acid
sequence, CDR3 amino acid sequence, full-length
amino acid sequence with CDRs capitalized,
full-length DNA sequence.
(SEQ ID NOS: 1-195)

KTGRSVI#RGDDT#VYFVNDCIV#evqlvesggglvqagdslrlscaasg
KTGRSVImgwfrqapgkerefvatitRGDDTtnyadsvkgrftisrddar
ntvylqmnslkpedtavyycaaVYFVNDCIVdywgqgtqvtvss#ATACC
ATGGAAGTTCAACTGGTTGAATCTGGTGGCGGTTTAGTTCAAGCCGGTGA
TAGCCTTCGTCTGAGCTGCGCCGCAAGCGGAAAGACCGGTCGGAGCGTCA
TTATGGGTTGGTTTCGCCAGGCACCTGGTAAAGAACGTGAATTTGTGGCA
ACCATTACCCGCGGGGATGACACTACCAACTATGCCGATTCTGTTAAAGG
TCGCTTCACTATTTCACGCGATGATGCGCGTAATACCGTGTACTTACAGA
TGAACTCACTGAAACCTGAGGATACCGCCGTTTATTATTGTGCGGCAGTC
TATTTTGTCAACGACTGCATTGTTGACTATTGGGGTCAGGGCACACAAGT
CACGGTCTCAAGCGGCGGAAGCTCTGGTGAACAGAAACTGATTTCAGAAG
KTGRSVI#RGDDT#IVLLTNLGLC#evqlvesggglvqag
dslrlscaasgKTGRSVImgwfrqapgkerefvatitRGDDTtnyadsvk
grftisrddarntvylqmnslkpedtavyycarlVLLTNLGLCdywgqgt
qvtvss#AGATATACCATGGAAGTTCAACTGGTTGAATCTGGTGGCGGTT
TAGTTCAAGCCGGTGATAGCCTTCGTCTGAGCTGCGCCGCAAGCGGAAAG
ACCGGTCGGAGCGTCATTATGGGTTGGTTTCGCCAGGCACCTGGTAAAGA
ACGTGAATTTGTGGCAACCATTACCCGCGGGGATGACACTACCAACTATG
CCGATTCTGTTAAAGGTCGCTTCACTATTTCACGCGATGATGCGCGTAAT
ACCGTGTACTTACAGATGAACTCACTGAAACCTGAGGATACCGCCGTTTA
TTATTGTGCGCGTATTGTGCTCCTGACTAACCTTGGCCTCTGCGACTATT
GGGGTCAGGGCACACAAGTCACGGTCTCAAGCGGCGGAAGCTCTGGTGAA
CAGAAACTGATTTCAGAAG
KTGRSVI#RGDDT#VYFVNDC
IV#evqlvesggglvqagdslrlscaasgKTGRSVImgwfrqapdkeref
vatitRGDDTtnyadsvkgrftisrddarntvylqmnslkpedtavyyca
aVYFVNDCIVdywgqgtqvtvss#ACCATGGAAGTTCAACTGGTTGAATC
TGGTGGCGGTTTAGTTCAAGCCGGTGATAGCCTTCGTCTGAGCTGCGCCG
CAAGCGGAAAGACCGGTCGGAGCGTCATTATGGGTTGGTTTCGCCAGGCA
CCTGATAAAGAACGTGAATTTGTGGCAACCATTACCCGCGGGGATGACAC
TACCAACTATGCCGATTCTGTTAAAGGTCGCTTCACTATTTCACGCGATG
ATGCGCGTAATACCGTGTACTTACAGATGAACTCACTGAAACCTGAGGAT
ACCGCCGTTTATTATTGTGCGGCAGTCTATTTTGTCAACGACTGCATTGT
TGACTATTGGGGTCAGGGCACACAAGTCACGGTCTCAAGCGGCGGAAGCT
CTGGTGAACAGAAACTGATTTCAGAAG
KTGRSVI#RGDDTVYFVNDCIV#evqlvesggglvqagdslrlscaasg
KTGRSVImgwfrq
apgkerefvatitRGDDTtnyadsvkgrftisrddarntvylqmnslkpe
dtavyycaaVYFVNDCIVaywgqgtqvtvss#ATATACCATGGAAGTTCA
ACTGGTTGAATCTGGTGGCGGTTTAGTTCAAGCCGGTGATAGCCTTCGTC
TGAGCTGCGCCGCAAGCGGAAAGACCGGTCGGAGCGTCATTATGGGTTGG
TTTCGCCAGGCACCTGGTAAAGAACGTGAATTTGTGGCAACCATTACCCG
CGGGGATGACACTACCAACTATGCCGATTCTGTTAAAGGTCGCTTCACTA
TTTCACGCGATGATGCGCGTAATACCGTGTACTTACAGATGAACTCACTG
AAACCTGAGGATACCGCCGTTTATTATTGTGCGGCAGTCTATTTTGTCAA
CGACTGCATTGTTGCCTATTGGGGTCAGGGCACACAAGTCACGGTCTCAA
GCGGCGGAAGCTCTGGTGAACAGAAACTGATTTCAGAAG
K
TGRSVI#RGDDT#VYFVNDCIV#evqlvesggglvqagdslrlscaasgK
TGRSVImgwfrqapgkerefvatitRGDDTtnyadsvkgrltisrddarn
tvylqmnslkpedtavyycaaVYFVNDCIVdywgqgtqvtvss#AGATAT
ACCATGGAAGTTCAACTGGTTGAATCTGGTGGCGGTTTAGTTCAAGCCGG
TGATAGCCTTCGTCTGAGCTGCGCCGCAAGCGGAAAGACCGGTCGGAGCG
TCATTATGGGTTGGTTTCGCCAGGCACCTGGTAAAGAACGTGAATTTGTG
GCAACCATTACCCGCGGGGATGACACTACCAACTATGCCGATTCTGTTAA
AGGTCGCCTCACTATTTCACGCGATGATGCGCGTAATACCGTGTACTTAC
AGATGAACTCACTGAAACCTGAGGATACCGCCGTTTATTATTGTGCGGCA
GTCTATTTTGTCAACGACTGCATTGTTGACTATTGGGGTCAGGGCACACA
AGTCACGGTCTCAAGCGGCGGAAGCTCTGGTGAACAGAAACTGATTTCAG
AAG
KTGRSVI#RGDDT#VYFVNDCIV#evqlvesggglvq
agdslrlscaasgKTGRSVImgwfrqapgkerefvatitRGDDTtnyvds
vkgrftisrddarntvylqmnslkpedtavyycaaVYFVNDCIVdywgqg
tqvtvss#GGAGATATACCATGGAAGTTCAACTGGTTGAATCTGGTGGCG
GTTTAGTTCAAGCCGGTGATAGCCTTCGTCTGAGCTGCGCCGCAAGCGGA
AAGACCGGTCGGAGCGTCATTATGGGTTGGTTTCGCCAGGCACCTGGTAA
AGAACGTGAATTTGTGGCAACCATTACCCGCGGGGATGACACTACCAACT
ATGTCGATTCTGTTAAAGGTCGCTTCACTATTTCACGCGATGATGCGCGT
AATACCGTGTACTTACAGATGAACTCACTGAAACCTGAGGATACCGCCGT
TTATTATTGTGCGGCAGTCTATTTTGTCAACGACTGCATTGTTGACTATT
GGGGTCAGGGCACACAAGTCACGGTCTCAAGCGGCGGAAGCTCTGGTGAA
CAGAAACTGATTTCAGAAG
KTGRSVI#RGDDT#VYFVNDC
IV#evqlvesggglvqagdslrlscaasgKTGRSVImgwfrqapgkeref
vatitRGDDTtnyadsvkgrftisrddarntvylqmntlkpedtavyyca
aVYFVNDCIVddwgqgtqvtvss#ATACCATGGAAGTTCAACTGGTTGAA
TCTGGTGGCGGTTTAGTTCAAGCCGGTGATAGCCTTCGTCTGAGCTGCGC
CGCAAGCGGAAAGACCGGTCGGAGCGTCATTATGGGTTGGTTTCGCCAGG
CACCTGGTAAAGAACGTGAATTTGTGGCAACCATTACCCGCGGGGATGAC
ACTACCAACTATGCCGATTCTGTTAAAGGTCGCTTCACTATTTCACGCGA
TGATGCGCGTAATACCGTGTACTTACAGATGAACACACTGAAACCTGAGG
ATACCGCCGTTTATTATTGTGCGGCAGTCTATTTTGTCAACGACTGCATT
GTTGACGATTGGGGTCAGGGCACACAAGTCACGGTCTCAAGCGGCGGAAG
CTCTGATGAACAGAAACGGATTTCAGAAG
KTGRSVI#RGD
DT#VYFVNDCIV#evqlfesggglvqagdslrlscaasgKTGRSVImgwf
rqapgkerefvatitRGDDTtnyadsvkgrftisrddarntvylqmnslk
pedtavyycaaVYFVNDCIVdywgqgtqvtvss#ATATACCATGGAAGTT
CAACTGTTTGAATCTGGTGGCGGTTTAGTTCAAGCCGGTGATAGCCTTCG
TCTGAGCTGCGCCGCAAGCGGAAAGACCGGTCGGAGCGTCATTATGGGTT
GGTTTCGCCAGGCACCTGGTAAAGAACGTGAATTTGTGGCAACCATTACC
CGCGGGGATGACACTACCAACTATGCCGATTCTGTTAAAGGTCGCTTCAC
TATTTCACGCGATGATGCGCGTAATACCGTGTACTTACAGATGAACTCAC
TGAAACCTGAGGATACCGCCGTTTATTATTGTGCGGCAGTCTATTTTGTC
AACGACTGCATTGTTGACTATTGGGGTCAGGGCACACAAGTCACGGTCTC
AAGCGGCGGAAGCTCTGGTGAACAGAAACTGATTTCAGAAG
KTGRSVI#RGDDT#VYFVNDCIV#evqlvesggglvqagdslrlscaas
gKTGRSVImgwfrqapgkerefvatitRGDDTtnyadsvkgrftisrdda
rntvylqmnslkpedtavyycaaVYFVNDCIVeyggqgtqvtvas#ATAC
CATGGAAGTTCAACTGGTTGAATCTGGTGGCGGTTTAGTTCAAGCCGGTG
ATAGCCTTCGTCTGAGCTGCGCCGCAAGCGGAAAGACCGGTCGGAGCGTC
ATTATGGGTTGGTTTCGCCAGGCACCTGGTAAAGAACGTGAATTTGTGGC
AACCATTACCCGCGGGGATGACACTACCAACTATGCCGATTCTGTTAAAG
GTCGCTTCACTATTTCACGCGATGATGCGCGTAATACCGTGTACTTACAG
ATGAACTCACTGAAACCTGAGGATACCGCCGTTTATTATTGTGCGGCAGT
CTATTTTGTCAACGACTGCATTGTTGAATATGGGGGTCAGGGCACACAAG
TCACGGTCGCAAGCGGCGGAAGCTCTGGTGAACAGAAACTGATTTCAGAA
G
KTGRSVI#RGDDT#VYFVNDCIV#evqlvepggglvqag
dslrlscaasgKTGRSVImgwfrqapgkerefaatitRGDDTtnyadsvk
grftisrddarntvylqmnslkpedtavyycaaVYFVNDCIVdywgqgtq
vtvss#ATATACCATGGAAGTTCAACTGGTTGAACCTGGTGGCGGTTTAG
TTCAAGCCGGTGATAGCCTTCGTCTGAGCTGCGCCGCAAGCGGAAAGACC
GGTCGGAGCGTCATTATGGGTTGGTTTCGCCAGGCACCTGGCAAAGAACG
TGAATTTGCGGCAACCATTACCCGCGGGGATGACACTACCAACTATGCCG
ATTCTGTTAAAGGTCGCTTCACTATTTCACGCGATGATGCGCGTAATACC
GTGTACTTACAGATGAACTCACTGAAACCTGAGGATACCGCCGTTTATTA
TTGTGCGGCAGTCTATTTTGTCAACGACTGCATTGTTGACTATTGGGGTC
AGGGCACACAAGTCACGGTCTCAAGCGGCGGAAGCTCTGGTGAACAGAAA
CTGATTTCAGAAG
KTGRSVI#RGDET#VYFVNDCIV#evq
lvesggdliqdgdslrlscaasgKTGRSVImgwfrqapgkerefaatitR
GDETtnyadsvkgrftisrddarntvylqmnslkpedtavyycaaVYFVN
DCIVdywcqgtqvtvsr#ATATACCATGGAAGTTCAACTGGTTGAATCTG
GTGGCGATTTAATTCAAGACGGTGATAGCCTTCGTCTGAGCTGCGCCGCA
AGCGGAAAGACCGGTCGGAGCGTCATTATGGGTTGGTTTCGCCAGGCACC
TGGCAAAGAACGTGAATTTGCGGCAACCATTACCCGCGGGGATGAAACTA
CCAACTATGCCGATTCTGTTAAAGGTCGCTTCACTATTTCACGCGATGAT
GCGCGTAATACCGTGTACTTACAGATGAACTCACTGAAACCTGAGGATAC
CGCCGTTTATTATTGTGCGGCAGTCTATTTTGTCAACGACTGCATTGTTG
ACTATTGGTGTCAGGGCACACAAGTCACGGTCTCACGCGGCGGAAGCTCT
TTTGAACAGAAACTGATTTCAGAAG
KTGRSVI#RGDDT#V
YFVNDCIV#evqlvesggglvqagdslrlrcaasgKTGRSVImgwfrqap
gkerefvatitRGDDTtnyadsvksrftisrddarntvylqmnslkpedt
avyycaaVYFVNDCIVdywcqgtqvtvss#ACCATGGAAGTTCAACTGGT
TGAATCTGGTGGCGGTTTAGTTCAAGCCGGTGATAGCCTTCGTCTGAGAT
GCGCCGCAAGCGGAAAGACCGGTCGGAGCGTCATTATGGGTTGGTTTCGC
CAGGCACCTGGTAAAGAACGTGAATTTGTGGCAACCATTACCCGCGGGGA
TGACACTACCAACTATGCCGATTCTGTTAAAAGTCGCTTCACTATTTCAC
GCGATGATGCGCGTAATACCGTGTACTTACAGATGAACTCACTGAAACCT
GAGGATACCGCCGTTTATTATTGTGCGGCAGTCTATTTTGTCAACGACTG
CATTGTTGACTATTGGTGTCAGGGCACACAAGTCACGGTCTCAAGCGGCG
GAAGCTCTTTTGAACAGACACTGATTTCAGAAG
IGERC
LF#AFSFR#VYFVNDCIV#evqlvesggglvqagdslrlscaasgIGERC
LFmgwfrqapgkerefvaaitAFSFRtyyadsvkgrftisrddarntvyl
qmnslkpedtavyycaaVYFVNDCIVdywgqgtqvtvss#GGAGATATAC
CATGGAAGTTCAACTGGTTGAATCTGGTGGCGGTTTAGTTCAAGCCGGTG
ATAGCCTTCGTCTGAGCTGCGCCGCAAGCGGAATCGGCGAGCGTTGTTTG
TTTATGGGTTGGTTTCGCCAGGCACCTGGTAAAGAACGTGAATTTGTGGC
AGCCATTACCGCGTTTAGTTTTCGGACCTACTATGCCGATTCTGTTAAAG
GTCGCTTCACTATTTCACGCGATGATGCGCGTAATACCGTGTACTTACAG
ATGAACTCACTGAAACCTGAGGATACCGCCGTTTATTATTGTGCGGCAGT
CTATTTTGTCAACGACTGCATTGTTGACTATTGGGGTCAGGGCACACAAG
TCACGGTCTCAAGCGGCGGAAGCTCTGGTGAACAGAAACTGATTTCAGAA
G
KTGRSVI#RGDDT#IVLLTNLGLC#evqlvesggglvqa
gdslrlscaasgKTGRSVImgwfrqapgkerefvatitRGDDTtnyadsv
kgrftisrddarntvylqmnslkpedtavyycarlVLLTNLGLCdywgqg
tqvtvsr#GAAGGAGATATACCATGGAAGTTCAACTGGTTGAATCTGGTG
GCGGTTTAGTTCAAGCCGGTGATAGCCTTCGTCTGAGCTGTGCCGCAAGC
GGAAAGACCGGTCGGAGCGTCATTATGGGTTGGTTTCGCCAGGCACCTGG
TAAAGAACGTGAATTTGTGGCAACCATTACCCGCGGGGATGACACTACCA
ACTATGCCGATTCTGTTAAAGGTCGCTTCACTATTTCACGCGATGATGCG
CGTAATACCGTGTACTTACAGATGAACTCACTGAAACCTGAGGATACCGC
CGTTTATTATTGTGCGCGTATTGTGCTCCTGACTAACCTTGGCCTCTGCG
ACTATTGGGGTCAGGGCACACAAGTCACGGTCTCACGCGGCGGAAGCTCT
GGTGAACAGAAACTGATTTCAGAAG
KTGRSVI#RGDDT#V
YFVNDCIV#evqlvesggglvqasdslrlscaasgKTGRSVImgwfrqap
gkerefvatitRGDDTtnyadsvkgrftisrddarntvylqmnslkpedt
avyycaaVYFVNDCIVdywgqgtqvtvss#ATATACCATGGAAGTTCAAC
TGGTTGAATCTGGTGGCGGTTTAGTTCAAGCCAGTGATAGCCTTCGTCTG
AGCTGCGCCGCAAGCGGAAAGACCGGTCGGAGCGTCATTATGGGTTGGTT
TCGCCAGGCACCTGGTAAAGAACGTGAATTTGTGGCAACCATTACCCGCG
GGGATGACACTACCAACTATGCCGATTCTGTTAAAGGTCGCTTCACTATT
TCACGCGATGATGCGCGTAATACCGTGTACTTACAGATGAACTCACTGAA
ACCTGAGGATACCGCCGTTTATTATTGTGCGGCAGTCTATTTTGTCAACG
ACTGCATTGTTGACTATTGGGGTCAGGGCACACAAGTCACGGTCTCAAGC
GGCGGAAGCTCTGGTGAACAGAAACTGATTTCAGAAG
KTG
RSVI#RGDDT#VYFVNDCIV#evqlvesggglvqagdslrlscaasgKTG
RSVImgwfrqapgkerefvatitRGDDTtnyadsvksrftisrddarntv
ylqmnslkpedtavyycaaVYFVNDCIVdywgqgtqvtvss#AGATATAC
CATGGAAGTTCAACTGGTTGAATCTGGTGGCGGTTTAGTTCAAGCCGGTG
ATAGCCTTCGTCTGAGCTGCGCCGCAAGCGGAAAGACCGGTCGGAGCGTC
ATTATGGGTTGGTTTCGCCAGGCACCTGGTAAAGAACGTGAATTTGTGGC
AACCATTACCCGCGGGGATGACACTACCAACTATGCCGATTCTGTTAAAA
GTCGCTTCACTATTTCACGCGATGATGCGCGTAATACCGTGTACTTACAG
ATGAACTCACTGAAACCTGAGGATACCGCCGTTTATTATTGTGCGGCAGT
CTATTTTGTCAACGACTGCATTGTTGACTATTGGGGTCAGGGCACACAAG
TCACGGTCTCAAGCGGCGGAAGCTCTGGTGAACAGAAACTGATTTCAGAA
G
KTGRSVI#RGDDT#VYFVNDCIV#kvqlvesggglvqag
dslrlscaasgKTGRSVImgwfrqapgkerefvatitRGDDTtnyadsvk
grftisrddarntvylqmnslkpedtavyycaaVYFVNDCIVdywgqgtq
vtvss#ATATACCATGAAAGTTCAACTGGTTGAATCTGGTGGCGGTTTAG
TTCAAGCCGGTGATAGCCTTCGTCTGAGCTGCGCCGCAAGCGGAAAGACC
GGTCGGAGCGTCATTATGGGTTGGTTTCGCCAGGCACCTGGTAAAGAACG
TGAATTTGTGGCAACCATTACCCGCGGGGATGACACTACCAACTATGCCG
ATTCTGTTAAAGGTCGCTTCACTATTTCACGCGATGATGCGCGTAATACC
GTGTACTTACAGATGAACTCACTGAAACCTGAGGATACCGCCGTTTATTA
TTGTGCGGCAGTCTATTTTGTCAACGACTGCATTGTTGACTATTGGGGTC
AGGGCACACAAGTCACGGTCTCAAGCGGCGGAAGCTCTGGTGAACAGAAA
CTGATTTCAGAAG
KTGRSVI#RGDDT#VYFVNDCIV#evq
lvesggglvqagdslrlscaasgKTGRSVImgwfrqapgkerefvatitR
GDDTtnyadsvkgrftisrddacntvylqmnslkpedtavyycaaVYFVN
DCIVdywgqgtqvtvss#ATACCATGGAAGTTCAACTGGTTGAATCTGGT
GGCGGTTTAGTTCAAGCCGGTGATAGCCTTCGTCTGAGCTGCGCCGCAAG
CGGAAAGACCGGTCGGAGCGTCATTATGGGTTGGTTTCGCCAGGCACCTG
GTAAAGAACGTGAATTTGTGGCAACCATTACCCGCGGGGATGACACTACC
AACTATGCCGATTCTGTTAAAGGTCGCTTCACTATTTCACGCGATGATGC
GTGTAATACCGTGTACTTACAGATGAACTCACTGAAACCTGAGGATACCG
CCGTTTATTATTGTGCGGCAGTCTATTTTGTCAACGACTGCATTGTTGAC
TATTGGGGTCAGGGCACACAAGTCACGGTCTCAAGCGGCGGAAGCTCTGG
TGAACAGAAACTGATTTCAGAAG
KTGRSVI#RGDDT#VYF
VNDCIV#evqlfesggglvqagdslrlscaasgKTGRSVImgwfrqapgk
erefvapitRGDDTtnyadsvkgrftisrddarntvylqmnslkpedtav
yycaaVYFVNDCIVdywgqgtqvtvss#ATATACCATGGAAGTTCAACTG
TTTGAATCTGGTGGCGGTTTAGTTCAAGCCGGTGATAGCCTTCGTCTGAG
CTGCGCCGCAAGCGGAAAGACCGGTAGGAGCGTCATTATGGGTTGGTTTC
GCCAGGCACCTGGTAAAGAACGTGAATTTGTGGCACCCATTACCCGCGGG
GATGACACTACCAACTATGCCGATTCTGTTAAAGGTCGCTTCACTATTTC
ACGCGATGATGCGCGTAATACCGTGTACTTACAGATGAACTCACTGAAAC
CTGAGGATACCGCCGTTTATTATTGTGCGGCAGTCTATTTTGTCAACGAC
TGCATTGTTGACTATTGGGGTCAGGGCACACAAGTCACGGTCTCAAGCGG
CGGAAGCTCTGGTGAACAGAAACTGATTTCAGAAG
KTGRS
VI#RGDDT#VYFVNDCIV#evqlvesggglvqagdnlrlscvasgKTGRS
VImgwfrqapgkerefvatitRGDDTtnyadsvkgrftisrddarntvyl
qmnslkpedtavyycaaVYFVNDCIVdywgqgtqvtvss#AGGAGATATA
CCATGGAAGTTCAACTGGTTGAATCTGGTGGCGGTTTAGTTCAAGCCGGT
GATAACCTTCGTCTGAGCTGCGTCGCAAGCGGAAAGACCGGTCGGAGCGT
CATTATGGGTTGGTTTCGCCAGGCACCTGGTAAAGAACGTGAATTTGTGG
CAACCATTACCCGCGGGGATGACACTACCAACTATGCCGATTCTGTTAAA
GGTCGCTTCACTATTTCACGCGATGATGCGCGTAATACCGTGTACTTACA
GATGAACTCACTGAAACCTGAGGATACCGCCGTTTATTATTGTGCGGCAG
TCTATTTTGTCAACGACTGCATTGTTGACTATTGGGGTCAGGGCACACAA
GTCACGGTCTCAAGCGGCGGAAGCTCTGGTGAACAGAAACTGATTTCAGA
AG
KTGRSVI#RGDDT#VYFVNDCIV#evqlvesggglvqa
gdslrlscaasgKTGRSVImgwfrqapgkerefvatltRGDDTtnyadsv
kgrftisrddarntvylqmnslkpedtavyycaaVYFVNDCIVdywgqgt
qvtvss#ACCATGGAAGTTCAACTGGTTGAATCTGGTGGCGGTTTAGTTC
AAGCCGGTGATAGCCTTCGTCTGAGCTGCGCCGCAAGCGGAAAGACCGGT
CGGAGCGTCATTATGGGTTGGTTTCGCCAGGCACCTGGTAAAGAACGTGA
ATTTGTGGCAACCCTTACCCGCGGGGATGACACTACCAACTATGCCGATT
CTGTTAAAGGTCGCTTCACTATTTCACGCGATGATGCGCGTAATACCGTG
TACTTACAGATGAACTCACTGAAACCTGAGGATACCGCCGTTTATTATTG
TGCGGCAGTCTATTTTGTCAACGACTGCATTGTTGACTATTGGGGTCAGG
GCACACAAGTCACGGTCTCAAGCGGCGGAAGCTCTGGTGAACAGAAACTG
ATTTCAGAAG
KTGRSVI#RGDDT#VYFVNDCIV#evqlve
sggglvqagdslrlscaargKTGRSVImgwfrqapgkerelvatitRGDD
TtnyadsvkgrftisrdearntvylqmnslkpedtavyycaaVYFVNDCI
Vaywgqgtqvtvss#ACCATGGAAGTTCAACTGGTTGAATCTGGTGGCGG
TTTAGTTCAAGCCGGTGATAGCCTTCGTCTGAGCTGCGCCGCAAGAGGAA
AGACCGGTCGAAGCGTCATTATGGGTTGGTTTCGCCAGGCACCTGGTAAA
GAACGTGAATTGGTGGCAACCATTACCCGCGGGGATGACACTACCAACTA
TGCCGATTCTGTTAAAGGTCGCTTCACTATTTCACGCGATGAGGCGCGTA
ATACCGTGTACTTACAGATGAACTCACTGAAACCTGAGGATACCGCCGTT
TATTATTGTGCGGCAGTCTATTTTGTCAACGACTGCATTGTTGCCTATTG
GGGCCAGGGCACACAAGTCACGGTCTCAAGCGGCGGAAGCTCTGGTGAAC
AGAAACTGATTTCAGAAG
KTGRSVI#RGDDT#VYFVNDCI
V#evqlvesggglvqagdrlrlscaasgKTGRSVImgwfrqapgkerefv
atitRGDDTtnyadsvkgrftisrddarntvylqmnslkpedtavyycaa
VYFVNDCIVdywgqgtqvtvss#GAAGGAGATATACCATGGAAGTTCAAC
TGGTTGAATCTGGTGGCGGTTTAGTTCAAGCCGGTGATCGCCTTCGTCTG
AGCTGCGCCGCAAGCGGAAAGACCGGTCGGAGCGTCATTATGGGTTGGTT
TCGCCAGGCACCTGGTAAAGAACGTGAATTTGTGGCAACCATTACCCGCG
GGGATGACACTACCAACTATGCCGATTCTGTTAAAGGTCGCTTCACTATT
TCACGCGATGATGCGCGTAATACCGTGTACTTACAGATGAACTCACTGAA
ACCTGAGGATACCGCCGTTTATTATTGTGCGGCAGTCTATTTTGTCAACG
ACTGCATTGTTGACTATTGGGGTCAGGGCACACAAGTCACGGTCTCAAGC
GGCGGAAGCTCTGGTGAACAGAAACTGATTTCAGAAG
KTG
RSVI#RGDDT#VYFVNDCIV#evqlvesggglvhagdslrlscaasgKTG
RSVImgwfrqapgkerefvatitRGDDTtnyadsvkgrftisrddarntv
ylqmnslkpedtavyycaaVYFVNDCIVdywgqgtqvtvsr#GAAGGAGA
TATACCATGGAAGTTCAACTGGTTGAATCTGGTGGCGGTTTAGTTCACGC
CGGTGATAGCCTTCGTCTGAGCTGCGCCGCAAGCGGAAAGACCGGTCGGA
GCGTCATTATGGGTTGGTTTCGCCAGGCACCTGGTAAAGAACGTGAATTT
GTGGCAACCATTACCCGCGGGGATGACACTACCAACTATGCCGATTCTGT
TAAAGGTCGCTTCACTATTTCACGCGATGATGCGCGTAATACCGTGTACT
TACAGATGAACTCACTGAAACCTGAGGATACCGCCGTTTATTATTGTGCG
GCAGTCTATTTTGTCAACGACTGCATTGTTGACTATTGGGGTCAGGGCAC
ACAAGTCACGGTCTCACGCGGCGGAAGCTCTTGTGAACAGAAACTGATTT
CAGAAG
KTGRSVI#RGDDT#IVLLTNLGLC#evqlvesgg
glvqagdrlrlscaasgKTGRSVImgwfrqapgkerefvatitRGDDTtn
yadsvkgrftisrddarntvylqmnslkpedtavyycvrlVLLTNLGLCd
ywgqgtqvtvss#GAAGGAGATATACCATGGAAGTTCAACTGGTTGAATC
TGGTGGCGGTTTAGTTCAAGCCGGTGATCGCCTTCGTCTGAGCTGCGCCG
CAAGCGGAAAGACCGGTCGGAGCGTCATTATGGGTTGGTTTCGCCAGGCA
CCTGGTAAAGAACGTGAATTTGTGGCAACCATTACCCGCGGGGATGACAC
TACCAACTATGCCGATTCTGTTAAAGGTCGCTTCACTATTTCACGCGATG
ATGCGCGTAATACCGTGTACTTACAGATGAACTCACTGAAACCTGAGGAT
ACCGCCGTTTATTATTGTGTGCGTATTGTGCTCCTGACTAACCTTGGCCT
TTGCGACTATTGGGGTCAGGGCACACAAGTCACGGTCTCAAGCGGCGGAA
GCTCTGGTGAAAAGAAACTGATTTCAGAAGCCAA
KTGRSV
I#RGDDT#VYFVNDCIV#evqlvesggglvqagdslrlscaasgKTGRSV
ImgwfrqapgkerefvatitRGDDTtnyadsvkgrftisrddarntvylq
mnslkpedtavyycaaVYFVNDCIVdywgqgthvtvss#ATACCATGGAA
GTTCAACTGGTTGAATCTGGTGGCGGTTTAGTTCAAGCCGGTGATAGCCT
TCGTCTGAGCTGCGCCGCAAGCGGAAAGACCGGTCGGAGCGTCATTATGG
GTTGGTTTCGCCAGGCACCTGGTAAAGAACGTGAATTTGTGGCAACCATT
ACCCGCGGGGATGACACTACCAACTATGCCGATTCTGTTAAAGGTCGCTT
CACTATTTCACGCGATGATGCGCGTAATACCGTGTACTTACAGATGAACT
CACTGAAACCTGAGGATACCGCCGTTTATTATTGTGCGGCAGTCTATTTT
GTCAACGACTGCATTGTTGACTATTGGGGTCAGGGCACACATGTCACGGT
CTCAAGCGGCGGAAGCTCTGGTGAACAGAAACTGATTTCAGAAGCCAA
KTGRSVI#RGDDT#VYFVNDCIV#evqlvesggglvqagdsl
rlscaasgKTGRSVImgwfrqapgkerefvatitRGDDTtnyadsvkgrf
tisredarntvylqmnslkpedtavyycaaVYFVNDCIVdywgqgtqvtv
ss#AGATATACCATGGAAGTTCAACTGGTTGAATCTGGTGGCGGTTTAGT
TCAAGCCGGTGATAGCCTTCGTCTGAGCTGCGCCGCAAGCGGAAAGACCG
GTCGGAGCGTCATTATGGGTTGGTTTCGCCAGGCACCTGGTAAAGAACGT
GAATTTGTGGCAACCATTACCCGCGGGGATGACACTACCAACTATGCCGA
TTCTGTTAAAGGTCGCTTCACTATTTCACGCGAAGATGCGCGTAATACCG
TGTACTTACAGATGAACTCACTGAAACCTGAGGATACCGCCGTTTATTAT
TGTGCGGCAGTCTATTTTGTCAACGACTGCATTGTTGACTATTGGGGTCA
GGGCACACAAGTCACGGTCTCAAGCGGCGGAAGCTCTGGTGAAAAGAAAC
TGATTTCAGAAGCCAA
KTGRSVI#RGDDT#VYFVNDCIV#
evqlvesggglvqagdslrlscaasgKTGRSVImgwfrqapgkerefvat
itRGDDTtnyadsvkgrftisrddarntvylqmnslkpedtavyycaaVY
FVNDCIVdywgqgtqgtvss#AGATATACCATGGAAGTTCAACTGGTTGA
ATCTGGTGGCGGTTTAGTTCAAGCCGGTGATAGCCTTCGTCTGAGCTGCG
CCGCAAGCGGAAAGACCGGTCGGAGCGTCATTATGGGTTGGTTTCGCCAG
GCACCTGGTAAAGAACGTGAATTTGTGGCAACCATTACCCGCGGGGATGA
CACTACCAACTATGCCGATTCTGTTAAAGGTCGCTTCACTATTTCACGCG
ATGATGCGCGTAATACCGTGTACTTACAGATGAACTCACTGAAACCTGAG
GATACCGCCGTTTATTATTGTGCGGCAGTCTATTTTGTCAACGACTGCAT
TGTTGACTATTGGGGTCAGGGCACACAAGGCACGGTCTCAAGCGGCGGAA
GCTCTGGTGAACAGAAACTGATTTCAGAAGCCAA
KTGRSV
I#RGDDT#VYFVNDCIV#evqlvesggglvqagdslrlscaasgKTGRSV
ImgwfrqapgkerefvatitRGDDTtnyadsvkgrftisrddardtvylq
mnslkpedtavyycaaVYFVNDCIVdywgqgtqvtvss#GGAGATATACC
ATGGAAGTTCAACTGGTTGAATCTGGTGGCGGTTTAGTTCAAGCCGGTGA
TAGCCTTCGTCTGAGCTGCGCCGCAAGCGGAAAGACCGGTCGGAGCGTCA
TTATGGGTTGGTTTCGCCAGGCACCTGGTAAAGAACGTGAATTTGTGGCA
ACCATTACCCGCGGGGATGACACTACCAACTATGCCGATTCTGTTAAAGG
TCGCTTCACTATTTCACGCGATGATGCGCGTGATACCGTGTACTTACAGA
TGAACTCACTGAAACCTGAGGATACCGCCGTTTATTATTGTGCGGCAGTC
TATTTTGTCAACGACTGCATTGTTGACTATTGGGGTCAGGGCACACAAGT
CACGGTCTCAAGCGGCGGAAGCTCTGGTGAACAGAAACTGATTTCAGAAG
CCAA
KTGRSVI#RGDDT#IVLLTNLGLC#evqlvesgggl
vqagdslrlscaasgKTGRSVImgwfrqapgkerefvatitRGDDTtnya
dsvkgrftisrddarntvylqmnslkpedtavyycarlVLLTNLGLCdyw
gqgtqgtvss#AGATATACCATGGAAGTTCAACTGGTTGAATCTGGTGGC
GGTTTAGTTCAAGCCGGTGATAGCCTTCGTCTGAGCTGCGCCGCAAGCGG
AAAGACCGGTCGGAGCGTCATTATGGGTTGGTTTCGCCAGGCACCTGGTA
AAGAACGTGAATTTGTGGCAACCATTACCCGCGGGGATGACACTACCAAC
TATGCCGATTCTGTTAAAGGTCGCTTCACTATTTCACGCGATGATGCGCG
TAATACCGTGTACTTACAGATGAACTCACTGAAACCTGAGGATACCGCCG
TTTATTATTGTGCGCGTATTGTGCTCCTGACTAACCTTGGCCTCTGCGAC
TATTGGGGTCAGGGCACACAAGGCACGGTCTCAAGCGGCGGAAGCTCTGG
TGAACAGAAACTGATTTCAGAAGCCAA
QSGKGCR#RGD
DT#VYFVNDCIV#evqlvesggglvqagdslrlscaasgQSGKGCRmgwf
rqapgkerefvatitRGDDTtnyadsvkgrftisrddarntvylqmnslk
pedtavyycaaVYFVNDCIVdywgqgtqgtvss#ATACCATGGAAGTTCA
ACTGGTTGAATCTGGTGGCGGTTTAGTTCAAGCCGGTGATAGCCTTCGTC
TGAGCTGCGCCGCAAGCGGACAGAGTGGCAAGGGTTGCCGGATGGGTTGG
TTTCGCCAGGCACCTGGTAAAGAACGTGAATTTGTGGCAACCATTACCCG
CGGGGATGACACTACCAACTATGCCGATTCTGTTAAAGGTCGCTTCACTA
TTTCACGCGATGATGCGCGTAATACCGTGTACTTACAGATGAACTCACTG
AAACCTGAGGATACCGCCGTTTATTATTGTGCGGCAGTCTATTTTGTCAA
CGACTGCATTGTTGACTATTGGGGTCAGGGCACACAAGGCACGGTCTCAA
GCGGCGGAAGCTCTGGTGAACAGAAACTGATTTCAGAAGCCAA
KTGRSVI#RGDDT#VYFVNDCIV#qvqlvesggglvqagdslrlsc
aasgKTGRSVImgwfrqapgkerefvatitRGDDTtnyadsvkgrftisr
ddarntvylqmnslkpedtavyycaaVYFVNDCIVdywgqgtqgtvss#A
AGGAGATATACCATGCAGGTTCAACTGGTTGAATCTGGTGGCGGTTTAGT
TCAAGCCGGTGATAGCCTTCGTCTGAGCTGCGCCGCAAGCGGAAAGACCG
GTCGGAGCGTCATTATGGGTTGGTTTCGCCAGGCACCTGGTAAAGAACGT
GAATTTGTGGCAACCATTACCCGCGGGGATGACACTACCAACTATGCCGA
TTCTGTTAAAGGTCGCTTCACTATTTCACGCGATGATGCGCGTAATACCG
TGTACTTACAGATGAACTCACTGAAACCTGAGGATACCGCCGTTTATTAT
TGTGCGGCAGTCTATTTTGTCAACGACTGCATTGTTGACTATTGGGGTCA
GGGCACACAAGGCACGGTCTCAAGCGGCGGAAGCTCTGGTGAACAGAAAC
TGATTTCAGAAGCCAA
KTGRSVI#RGDDT#VYFVNDCIVevqlvesggglvkagdslrlscaasgK
TGRSVImgwfrqapgkerefva
titRGDDTtnyadsvkgrftisrddarntvylqmnslkpedtavyycaaV
YFVNDCIVdywgqgtqvtvss#GGAGATATACCATGGAAGTTCAACTGGT
TGAATCTGGTGGCGGTTTAGTTAAAGCCGGTGATAGCCTTCGTCTGAGCT
GCGCCGCAAGCGGAAAGACCGGTCGGAGCGTCATTATGGGTTGGTTTCGC
CAGGCACCTGGTAAAGAACGTGAATTTGTGGCAACCATTACCCGCGGGGA
TGACACTACCAACTATGCCGATTCTGTTAAAGGTCGCTTCACTATTTCAC
GCGATGATGCGCGTAATACCGTGTACTTACAGATGAACTCACTGAAACCT
GAGGATACCGCCGTTTATTATTGTGCGGCAGTCTATTTTGTCAACGACTG
CATTGTTGACTATTGGGGTCAGGGCACACAAGTCACGGTCTCAAGCGGCG
GAAGCTCTGGTGAACAGAAACTGATTTCAGAAGCCAA
K
TGRSVL#RGDDT#IVLLTNLGLC#evqlvesggglvqagdslrlscaasg
KTGRSVLmgwfrqapgkerefvatitRGDDTtnyadsvkgrftisrddar
ntvylqmnslkpedtavyycarlVLLTNLGLCdywgqgtqvtvss#GGAG
ATATACCATGGAAGTTCAACTGGTTGAATCTGGTGGCGGTTTAGTTCAAG
CCGGTGATAGCCTTCGTCTGAGCTGCGCCGCAAGCGGAAAGACCGGTCGG
AGCGTCCTTATGGGTTGGTTTCGCCAGGCACCTGGTAAAGAACGTGAATT
TGTGGCAACCATTACCCGCGGGGATGACACTACCAACTATGCCGATTCTG
TTAAAGGTCGCTTCACTATTTCACGCGATGATGCGCGTAATACCGTGTAC
TTACAGATGAACTCACTGAAACCTGAGGATACCGCCGTTTATTATTGTGC
GCGTATTGTGCTCCTGACTAACCTTGGCCTCTGCGACTATTGGGGTCAGG
GCACACAGGTCACGGTCTCAAGCGGCGGAAGCTCTGGTGAACAGAAACTG
ATTTCAGAAGCCAA
KTGRSVI#RGDDT#VYFVNDCIV#e
vqlvesggvlvqagdslrlscaasgKTGRSVImgwfrqapgkerefvati
tRGDDTtnyadsvkgrftisrddarntvylqmnslkpedtavyycaaVYF
VNDCIVdywgqgtqvtvss#AAGGAGATATACCATGGAAGTTCAACTGGT
TGAATCTGGTGGCGTTTTAGTTCAAGCCGGTGATAGCCTTCGTCTGAGCT
GCGCCGCAAGCGGAAAGACCGGTCGGAGCGTCATTATGGGTTGGTTTCGC
CAGGCACCTGGTAAAGAACGTGAATTTGTGGCAACCATTACCCGCGGGGA
TGACACTACCAACTATGCCGATTCTGTTAAAGGTCGCTTCACTATTTCAC
GCGATGATGCGCGTAATACCGTGTACTTACAGATGAACTCACTGAAACCT
GAGGATACCGCCGTTTATTATTGTGCGGCAGTCTATTTTGTCAACGACTG
CATTGTTGACTATTGGGGTCAGGGCACACAAGTCACGGTCTCAAGCGGCG
GAAGCTCTGGTGAACAGAAACTGATTTCAGAAGCCAA
KT
GRSVI#RGDDT#VYFVNDCIV#qvqlqesggglvqagdslrlscaasgKT
GRSVImgwfrqapgkerefvatitRGDDTtnyadsvkgrftisrddarnt
vylqmnslkpedtavyycaaVYFVNDCIVdywgqgtqgtvss#ACCATGC
AGGTTCAACTGCAAGAATCTGGTGGCGGTTTAGTTCAAGCCGGTGATAGC
CTTCGTCTGAGCTGCGCCGCAAGCGGAAAGACCGGTCGGAGCGTCATTAT
GGGTTGGTTTCGCCAGGCACCTGGTAAAGAACGTGAATTTGTGGCAACCA
TTACCCGCGGGGATGACACTACCAACTATGCCGATTCTGTTAAAGGTCGC
TTCACTATTTCACGCGATGATGCGCGTAATACCGTGTACTTACAGATGAA
CTCACTGAAACCTGAGGATACCGCCGTTTATTATTGTGCGGCAGTCTATT
TTGTCAACGACTGCATTGTTGACTATTGGGGTCAGGGCACACAAGGCACG
GTCTCAAGCGGCGGAAGCTCTGGTGAAAAGAAACTGATTTCAGAAGCCAA
KTGRSVL#RGDDT#VYFVNDCIV#evqlvesggglvqa
gdslrlscaasgKTGRSVLmgwfrqapgkerefvatitRGDDTtnyadsv
kgrftisrddarntvylqmnslkpedtavyycaaVYFVNDCIVdywgqgt
qvtvss#GGAGATATACCATGGAAGTTCAACTGGTTGAATCTGGTGGCGG
TTTAGTTCAAGCCGGTGATAGCCTTCGTCTGAGCTGCGCCGCAAGCGGAA
AGACCGGTCGGAGCGTCCTTATGGGTTGGTTTCGCCAGGCACCTGGTAAA
GAACGTGAATTTGTGGCAACCATTACCCGCGGGGATGACACTACCAACTA
TGCCGATTCTGTTAAAGGTCGCTTCACTATTTCACGCGATGATGCGCGTA
ATACCGTGTACTTACAGATGAACTCACTGAAACCTGAGGATACCGCCGTT
TATTATTGTGCGGCAGTCTATTTTGTCAACGACTGCATTGTTGACTATTG
GGGTCAGGGCACACAAGTCACGGTCTCAAGCGGCGGAAGCTCTGGTGAAC
AGAAACTGATTTCAGAAGCCAA
KTGRSVI#RGDDT#VYF
VNDCIV#evqlvesggglvqagdslrlscaasgKTGRSVImgwfrqapgk
erefvatitRGDDTtnyadsvkgrftisrddarntvylqmnslkpediav
yycaaVYFVNDCIVdywgqgtqgtvss#AGGAGATATACCATGGAAGTTC
AACTGGTTGAATCTGGTGGCGGTTTAGTTCAAGCCGGTGATAGCCTTCGT
CTGAGCTGCGCCGCAAGCGGAAAGACCGGTCGGAGCGTCATTATGGGTTG
GTTTCGCCAGGCACCTGGTAAAGAACGTGAATTTGTGGCAACCATTACCC
GCGGGGATGACACTACCAACTATGCCGATTCTGTTAAAGGTCGCTTCACT
ATTTCACGCGATGATGCGCGTAATACCGTGTACTTACAGATGAACTCACT
GAAACCTGAGGATATCGCCGTTTATTATTGTGCGGCAGTCTATTTTGTCA
ACGACTGCATTGTTGACTATTGGGGTCAGGGCACACAAGGCACGGTCTCA
AGCGGCGGAAGCTCTGGTGAAAAGAAACTGATTTCAGAAGCCAA
KTGRSVI#RGDDT#IVLLTNLGLC#evqlvesggglvqagdslrl
scaasgKTGRSVImgwfrqapgkerefvatitRGDDTtnyadsvkgrfti
srddacntvylqmnslkpedtavyycarlVLLTNLGLCdywgqgtqvtvs
s#ACCATGGAAGTTCAACTGGTTGAATCTGGTGGCGGTTTAGTTCAAGCC
GGTGATAGCCTTCGTCTGAGCTGCGCCGCAAGCGGAAAGACCGGTCGGAG
CGTCATTATGGGTTGGTTTCGCCAGGCACCTGGTAAAGAACGTGAATTTG
TGGCAACCATTACCCGCGGGGATGACACTACCAACTATGCCGATTCTGTT
AAAGGTCGCTTCACTATTTCACGCGATGATGCGTGTAATACCGTGTACTT
ACAGATGAACTCACTGAAACCTGAGGATACCGCCGTTTATTATTGTGCGC
GTATTGTGCTCCTGACTAACCTTGGCCTCTGCGACTATTGGGGTCAGGGC
ACACAAGTCACGGTCTCAAGCGGCGGAAGCTCTGGTGAACAGAAACTGAT
TTCAGAAGCCAA

TABLE 2
Sequences belonging to cluster SR2.
Each line in the table represents one sequence,
both segments and full length of the sequence
are shown. Shown items are divided by “#” and in
the order from start to end of each line is:CDR1
amino acid sequence, CDR2 amino acid sequence,
CDR3 amino acid sequence, full-length amino acid
sequence with CDRs capitalized, full-length
DNA sequence.
(SEQ ID NOS: 196-400)

AFGSNVF#PHHTV#AIYRNASVL#evqlvesggglvqagdslrlscaasg
AFGSNVFmgwfrqapgkerefvasitPHHTVtdyadsvrgrfsisadsak
ntvylqmnslkpedtavyycaaAIYRNASVLdywgqgtqvtvss#ATACC
ATGGAAGTTCAACTGGTTGAATCTGGTGGCGGTTTAGTTCAAGCCGGTGA
TAGCCTTCGTCTGAGCTGCGCCGCAAGCGGAGCTTTCGGTTCTAACGTTT
TCATGGGTTGGTTTCGCCAGGCACCTGGTAAAGAACGTGAATTTGTGGCA
TCCATTACCCCTCATCACACGGTCACCGACTATGCCGATTCTGTTCGTGG
TCGCTTCAGCATTTCAGCAGATTCTGCGAAGAATACCGTGTACTTACAGA
TGAACTCACTGAAACCTGAGGATACCGCCGTTTATTATTGTGCGGCAGCC
ATTTATAGGAACGCGTCGGTTTTGGACTATTGGGGTCAGGGCACACAAGT
CACGGTCTCAAGCGGCGGAAGCTCTGGTGAACAGAAACTGATTTCAGAAG
AFGSNVF#PHHTV#AIYRNASVL#evqlfesggglvqagdslrlscaasg
AFGSNVFmgwfrqapgkerefvasitPHHTVtdyadsvrgrfsisadsak
ntvylqmnslkpedtavyycaaAIYRNASVLdywgqgtqvtvss#AGATA
TACCATGGAAGTTCAACTGTTTGAATCTGGTGGCGGTTTAGTTCAAGCCG
GTGATAGCCTTCGTCTGAGCTGCGCCGCAAGCGGAGCTTTCGGTTCTAAC
GTTTTCATGGGTTGGTTTCGCCAGGCACCTGGTAAAGAACGTGAATTTGT
GGCATCCATTACCCCTCATCACACGGTCACCGACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGGC
AGCCATTTATAGGAACGCGTCGGTTTTGGACTATTGGGGTCAGGGCACAC
AAGTCACGGTCTCAAGCGGCGGAAGCTCTGGTGAACAGAAACTGATTTCA
GAAG
AFGSNVF#PHHTV#AIYRNASVL#evqlvesggglvqagdrlrlscaasg
AFGSNVFmgwfrqapgkerefvasitPHHTVtdyadsvrgrfsisadsak
ntvylqmnslkpedtavyycaaAIYRNASVLdywgqgtqvtvss#GAAGG
AGATATACCATGGAAGTTCAACTGGTTGAATCTGGTGGCGGTTTAGTTCA
AGCCGGTGATCGCCTTCGTCTGAGCTGCGCCGCAAGCGGAGCTTTCGGTT
CTAACGTTTTCATGGGTTGGTTTCGCCAGGCACCTGGTAAAGAACGTGAA
TTTGTGGCATCCATTACCCCTCATCACACGGTCACCGACTATGCCGATTC
TGTTCGTGGTCGCTTCAGCATTTCAGCAGATTCTGCGAAGAATACCGTGT
ACTTACAGATGAACTCACTGAAACCTGAGGATACCGCCGTTTATTATTGT
GCAGCAGCCATTTATAGGAACGCGTCGGTTTTGGACTATTGGGGTCAGGG
CACACAAGTCACGGTCTCAAGCGGCGGAAGCTCTGGTGAACAGAAACTGA
TTTCAGAAG
AFGSNVF#PHHTV#AIYRNASVL#evqlvesggglvqagdslrlscaasg
AFGSNVFmgwfrqapgkerefvasitPHHTVtdyadsvrgrfsisadsak
ntvylqmnslkpedtavyycvaAIYRNASVLdywgqgtqvtvss#ATACC
ATGGAAGTTCAACTGGTTGAATCTGGTGGCGGTTTAGTTCAAGCCGGTGA
TAGCCTTCGTCTGAGCTGCGCCGCAAGCGGAGCTTTCGGTTCTAACGTTT
TCATGGGTTGGTTTCGCCAGGCACCTGGTAAAGAACGTGAATTTGTGGCA
TCCATTACCCCTCATCACACGGTCACCGACTATGCCGATTCTGTTCGTGG
TCGCTTCAGCATTTCAGCAGATTCTGCGAAGAATACCGTGTACTTACAGA
TGAACTCACTGAAACCTGAGGATACCGCCGTTTATTATTGTGTGGCAGCC
ATTTATAGGAACGCGTCGGTTTTGGACTATTGGGGTCAGGGCACACAAGT
CACGGTCTCAAGCGGCGGAAGCTCTGGTGAACAGAAACTGATTTCAGAAG
AFGSNVF#PHHTV#AIYRNASVL#evqlvesggglvqagdslrlrcaasg
AFGSNVFmgwfrqapgkerefvasitPHHTVtdyadsvrgrfsisadsak
ntvylqmnslkpedtavyycaaAIYRNASVLdywgqgtqvtvss#AGGAG
ATATACCATGGAAGTTCAACTGGTTGAATCTGGTGGCGGTTTAGTTCAAG
CCGGTGATAGCCTTCGTCTGCGCTGCGCCGCAAGCGGAGCTTTCGGTTCT
AACGTTTTCATGGGTTGGTTTCGCCAGGCACCTGGTAAAGAACGTGAATT
TGTGGCATCCATTACCCCTCATCACACGGTCACCGACTATGCCGATTCTG
TTCGTGGTCGCTTCAGCATTTCAGCAGATTCTGCGAAGAATACCGTGTAC
TTACAGATGAACTCACTGAAACCTGAGGATACCGCCGTTTATTATTGTGC
GGCAGCCATTTATAGGAACGCGTCGGTTTTGGACTATTGGGGTCAGGGCA
CACAAGTCACGGTCTCAAGCGGCGGAAGCTCTGGTGAACAGAAACTGATT
TCAGAAG
AFGSNVF#PHHTV#AIYRNASVL#evqlvesggglvqagdslrlscaasg
AFGSNVFmgwfrqapgkerefvasitPHHTVtdyadsvrgrfsisadsak
ntvylqmnslkpedtavyycaaAIYRNASVLaywgqgtqvtvss#ATATA
CCATGGAAGTTCAACTGGTTGAATCTGGTGGCGGTTTAGTTCAAGCCGGT
GATAGCCTTCGTCTGAGCTGCGCCGCAAGCGGAGCTTTCGGTTCTAACGT
TTTCATGGGTTGGTTTCGCCAGGCACCTGGTAAAGAACGTGAATTTGTGG
CATCCATTACCCCTCATCACACGGTCACCGACTATGCCGATTCTGTTCGT
GGTCGCTTCAGCATTTCAGCAGATTCTGCGAAGAATACCGTGTACTTACA
GATGAACTCACTGAAACCTGAGGATACCGCCGTTTATTATTGTGCGGCAG
CCATTTATAGGAACGCGTCGGTTTTGGCCTATTGGGGTCAGGGCACACAA
GTCACGGTCTCAAGCGGCGGAAGCTCTGGTGAACAGAAACTGATTTCAGA
AG
AFGSNVF#PHHTV#AIYRNASVL#evqlvesggglvqagdslrlscaasg
AFGSNVFmgwfrqapgkerefvasitPHHTVtdyadsvrgrfsisadsak
itvylqmnslkpedtavyycaaAIYRNASVLdywgqgtqvtvss#ACCAT
GGAAGTTCAACTGGTTGAATCTGGTGGCGGTTTAGTTCAAGCCGGTGATA
GCCTTCGTCTGAGCTGCGCCGCAAGCGGAGCTTTCGGTTCTAACGTTTTC
ATGGGTTGGTTTCGCCAGGCACCTGGTAAAGAACGTGAATTTGTGGCATC
CATTACCCCTCATCACACGGTCACCGACTATGCCGATTCTGTTCGTGGTC
GCTTCAGCATTTCAGCAGATTCTGCGAAGATTACCGTGTACTTACAGATG
AACTCACTGAAACCTGAGGATACCGCCGTTTATTATTGTGCGGCAGCCAT
TTATAGGAACGCGTCGGTTTTGGACTATTGGGGTCAGGGCACACAAGTCA
CGGTCTCAAGCGGCGGAAGCTCTGGTGAACAGAAACTGATTTCAGAAG
AFGSNVF#PHHTV#AIYRNASVL#evqlvdsggglvqagdrlrlscaasg
AFGSNVFmgwfrqapgkerefvasitPHHTVtdyadsvrgrfsisadsak
ntvylqmnslkpedtavyycaaAIYRNASVLdywgqgtqvtvss#GAAGG
AGATATACCATGGAAGTTCAACTGGTTGACTCTGGTGGCGGTTTAGTTCA
AGCCGGTGATCGCCTTCGTCTGAGCTGCGCCGCAAGCGGAGCTTTTGGTT
CTAACGTTTTCATGGGTTGGTTTCGCCAGGCACCTGGTAAAGAACGTGAA
TTTGTGGCATCCATTACCCCTCATCACACGGTCACCGACTATGCCGATTC
TGTTCGTGGTCGCTTCAGCATTTCAGCAGATTCTGCGAAGAATACCGTGT
ACTTACAGATGAACTCACTGAAACCTGAGGATACCGCCGTTTATTATTGT
GCGGCAGCCATTTATAGGAACGCGTCGGTTTTGGACTATTGGGGTCAGGG
CACACAAGTCACGGTCTCAAGCGGCGGAAGCTCTGGTGAACAGAAACTGA
TTTCAGAAG
AFGSNVF#PHHTV#AIYRNASVL#evqlvesggglvqagdslrlscaasg
AFGSNVFmgwfrqapgkerefvasitPHHTVtdyadsvrgrfsisadsak
ntvylqmnslkpedtavyygaaAIYRNASVLdyggqgtqvtvss#GGAGA
TATACCATGGAAGTTCAACTGGTTGAATCTGGTGGCGGTTTAGTTCAAGC
CGGTGATAGCCTTCGTCTGAGCTGCGCCGCAAGCGGAGCTTTCGGTTCTA
ACGTTTTCATGGGTTGGTTTCGCCAGGCACCTGGTAAAGAACGTGAATTT
GTGGCATCCATTACCCCTCATCACACGGTCACCGACTATGCCGATTCTGT
TCGTGGTCGCTTCAGCATTTCAGCAGATTCTGCGAAGAATACCGTGTACT
TACAGATGAACTCACTGAAACCTGAGGATACCGCCGTTTATTATGGTGCG
GCAGCCATTTATAGGAACGCGTCGGTTTTGGACTATGGGGGTCAGGGCAC
ACAAGTCACGGTCTCAAGCGGCGGAAGCTCTGGTGAACAGAAACTGATTT
CAGAAG
AFGSNVF#PHHTV#AIYRNASGL#evqlvesggglvqagdslrlscaasg
AFGSNVFmgwfrqapgkerefvasitPHHTVtdyadsvrgrfsisadsak
ntvylqmnslkpedtavyycaaAIYRNASGLdywgqgtqvtvss#ATACC
ATGGAAGTTCAACTGGTTGAATCTGGTGGCGGTTTAGTTCAAGCCGGTGA
TAGCCTTCGTCTGAGCTGCGCCGCAAGCGGAGCTTTCGGTTCTAACGTTT
TCATGGGTTGGTTTCGCCAGGCACCTGGTAAAGAACGTGAATTTGTGGCA
TCCATTACCCCTCATCACACGGTCACCGACTATGCCGATTCTGTTCGTGG
TCGCTTCAGCATTTCAGCAGATTCTGCGAAGAATACCGTGTACTTACAGA
TGAACTCACTGAAACCTGAGGATACCGCCGTTTATTATTGTGCGGCAGCC
ATTTATAGGAACGCGTCGGGTTTGGACTATTGGGGTCAGGGCACACAAGT
CACGGTCTCAAGCGGCGGAAGCTCTGGTGAACAGAAACTGATTTCAGAAG
AFGSNVF#PHHTV#AIYRNASVL#evqlvesggglvqagdslrlscaasg
AFGSNVFmgwfrqapgkerefvasitPHHTVtdyadsvrgrfsisadsak
ntvylqmnslkpedtavyyceaAIYRNASVLdywgqgtqvtvss#ACCAT
GGAAGTTCAACTGGTTGAATCTGGTGGCGGTTTAGTTCAAGCCGGTGATA
GCCTTCGTCTGAGCTGCGCCGCAAGCGGAGCTTTCGGTTCTAACGTTTTC
ATGGGTTGGTTTCGCCAGGCACCTGGTAAAGAACGTGAATTTGTGGCATC
CATTACCCCTCATCACACGGTCACCGACTATGCCGATTCTGTTCGTGGTC
GCTTCAGCATTTCAGCAGATTCTGCGAAGAATACCGTGTACTTACAGATG
AACTCACTGAAACCTGAGGATACCGCCGTTTATTATTGTGAGGCAGCCAT
TTATAGGAACGCGTCGGTTTTGGACTATTGGGGTCAGGGCACACAAGTCA
CGGTCTCAAGCGGCGGAAGCTCTGGTGAACAGAAACTGATTTCAGAAG
AFGSNVF#PHHTV#AIYRNASVL#evqlvesggglvqagdsirlscaasg
AFGSNVFmgwfrqapgkerefvasitPHHTVtdyadsvrgrfsisadsak
ntvylqmnslkpedtavyycaaAIYRNASVLdywgqgtqvtvss#GAAGG
AGATATACCATGGAAGTTCAACTGGTTGAATCTGGTGGCGGTTTAGTTCA
AGCCGGTGATAGCATTCGTCTGAGCTGCGCCGCAAGCGGAGCTTTCGGTT
CTAACGTTTTCATGGGTTGGTTTCGCCAGGCACCTGGTAAAGAACGTGAA
TTTGTGGCATCCATTACCCCTCATCACACGGTCACCGACTATGCCGATTC
TGTTCGTGGTCGCTTCAGCATTTCAGCAGATTCTGCGAAGAATACCGTGT
ACTTACAGATGAACTCACTGAAACCTGAGGATACCGCCGTTTATTATTGT
GCGGCAGCCATTTATAGGAACGCGTCGGTTTTGGACTATTGGGGTCAGGG
CACACAAGTCACGGTCTCAAGCGGCGGAAGCTCTGGTGAACAGAAACTGA
TTTCAGAAG
AFGSNVF#PHHTV#AIYRNASVL#evqlfesggglvqagdslrlscaasg
AFGSNVFmgwfrqapgkerefvasitPHHTVtdyadsvrgrfsisadsak
ntvylqmnslkpedtavyycaaAIYRNASVLgywgqgtqvtvss#AGATA
TACCATGGAAGTTCAACTGTTTGAATCTGGTGGCGGTTTAGTTCAAGCCG
GTGATAGCCTTCGTCTGAGCTGCGCCGCAAGCGGAGCTTTCGGTTCTAAC
GTTTTCATGGGTTGGTTTCGCCAGGCACCTGGTAAAGAACGTGAATTTGT
GGCATCCATTACCCCTCATCACACGGTCACCGACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGGC
AGCCATTTATAGGAACGCGTCGGTTTTGGGCTATTGGGGTCAGGGCACAC
AAGTCACGGTCTCAAGCGGCGGAAGCTCTGGTGAACAGAAACTGATTTCA
GAAG
AFGSNVF#PHHTV#AIYRNASVL#evqlvesggglvkagdslrlscaasg
AFGSNVFmgwfrqapgkerefvasitPHHTVtdyadsvrgrfsisadsak
ntvylqmnslkpedtavyycaaAIYRNASVLdywgqgtqvtvss#GGAGA
TATACCATGGAAGTTCAACTGGTTGAATCTGGTGGCGGTTTAGTTAAAGC
CGGTGATAGCCTTCGTCTGAGCTGCGCCGCAAGCGGAGCTTTCGGTTCTA
ACGTTTTCATGGGTTGGTTTCGCCAGGCACCTGGTAAAGAACGTGAATTT
GTGGCATCCATTACCCCTCATCACACGGTCACCGACTATGCCGATTCTGT
TCGTGGTCGCTTCAGCATTTCAGCAGATTCTGCGAAGAATACCGTGTACT
TACAGATGAACTCACTGAAACCTGAGGATACCGCCGTTTATTATTGTGCG
GCAGCCATTTATAGGAACGCGTCGGTTTTGGACTATTGGGGTCAGGGCAC
ACAAGTCACGGTCTCAAGCGGCGGAAGCTCTGGTGAACAGAAACTGATTT
CAGAAG
AFGSNVF#PHHTV#AIYRNASVL#evqlvdsggglvhagdrlrlscaasg
AFGSNVFmgwfrqapgkerefvasitPHHTVtdyadsvrgrfsisadsak
ntvylqmnslkpedtavyycaaAIYRNASVLdywgqgtqvtvss#GAAGG
AGATATACCATGGAAGTTCAACTGGTTGACTCTGGTGGCGGTTTAGTTCA
CGCCGGTGATCGCCTTCGTCTGAGCTGCGCCGCAAGCGGAGCTTTCGGTT
CTAACGTTTTCATGGGTTGGTTTCGCCAGGCACCTGGTAAAGAACGTGAA
TTTGTGGCATCCATTACCCCTCATCACACGGTCACCGACTATGCCGATTC
TGTTCGTGGTCGCTTCAGCATTTCAGCAGATTCTGCGAAGAATACCGTGT
ACTTACAGATGAACTCACTGAAACCTGAGGATACCGCCGTTTATTATTGT
GCGGCAGCCATTTATAGGAACGCGTCGGTTTTGGACTATTGGGGTCAGGG
CACACAAGTCACGGTCTCAAGCGGCGGAAGCTCTGGTGAACAGAAACTGA
TTTCAGAAG
AFGSNVF#PHHTV#AIYRDASVL#evqlvesggglvqagdslrlscaasg
AFGSNVFmgwfrqapgkerefvasitPHHTVtdyadsvrgrfsisadsak
ntvylqmnslkpedtavyycaaAIYRDASVLdywgqgtqvtvss#AGGAG
ATATACCATGGAAGTTCAACTGGTTGAATCTGGTGGCGGTTTAGTTCAAG
CCGGTGATAGCCTTCGTCTGAGCTGCGCCGCAAGCGGAGCTTTCGGTTCT
AACGTTTTCATGGGTTGGTTTCGCCAGGCACCTGGTAAAGAACGTGAATT
TGTGGCATCCATTACCCCTCATCACACGGTCACCGACTATGCCGATTCTG
TTCGTGGTCGCTTCAGCATTTCAGCAGATTCTGCGAAGAATACCGTGTAC
TTACAGATGAACTCACTGAAACCTGAGGATACCGCCGTTTATTATTGTGC
GGCAGCCATTTATAGGGACGCGTCGGTTTTGGACTATTGGGGTCAGGGCA
CACAAGTCACGGTCTCAAGCGGCGGAAGCTCTGGTGAACAGAAACTGATT
TCAGAAG
AFGSNVF#PHHTF#AIYRNASVL#evqlvesggglvqagdslrlscaasg
AFGSNVFmgwfrqapgkerefvasitPHHTFtdyadsvrgrfsisadsak
ntvylqmnslkpedtavyycaaAIYRNASVLdywgqgtqvtvss#AGATA
TACCATGGAAGTTCAACTGGTTGAATCTGGTGGCGGTTTAGTTCAAGCCG
GTGATAGCCTTCGTCTGAGCTGCGCCGCAAGCGGAGCTTTCGGTTCTAAC
GTTTTCATGGGTTGGTTTCGCCAGGCACCTGGTAAAGAACGTGAATTTGT
GGCATCCATTACCCCTCATCACACGTTCACCGACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGGC
AGCCATTTATAGGAACGCGTCGGTTTTGGACTATTGGGGTCAGGGCACAC
AAGTCACGGTCTCAAGCGGCGGAAGCTCTGGTGAACAGAAACTGATTTCA
GAAG
AFGSNVF#PHHTV#AIYRNASVL#evqlvesggglvqagdslrlscaasg
AFGSNVFmgwfrqapgkerefvasitPHHTVtdyadsvrgrfsisadsak
ntvylqmnslkpedtavyycaaAIYRNASVLaywvqgtqvtvss#AGATA
TACCATGGAAGTTCAACTGGTTGAATCTGGTGGCGGTTTAGTTCAAGCCG
GTGATAGCCTTCGTCTGAGCTGCGCCGCAAGCGGAGCTTTCGGTTCTAAC
GTTTTCATGGGTTGGTTTCGCCAGGCACCTGGTAAAGAACGTGAATTTGT
GGCATCCATTACCCCTCATCACACGGTCACCGACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGGC
AGCCATTTATAGGAACGCGTCGGTTTTGGCCTATTGGGTTCAGGGCACAC
AAGTCACGGTCTCAAGCGGCGGAAGCTCTGTTGAACAGAAACTGATTTCA
GAAG
AFGSNVF#PHHTV#AIYRNASVL#evqlvesggglvqagdslrlscaasg
AFGSNVFmgwfrqapgkergfvasitPHHTVtdyadsvrgrfsisadsak
ntvylqmnslkpedtavyycaaAIYRNASVLdywgqgtqvtvss#GAAGG
AGATATACCATGGAAGTTCAACTGGTTGAATCTGGTGGCGGTTTAGTTCA
AGCCGGTGATAGCCTTCGTCTGAGCTGCGCCGCAAGCGGAGCTTTCGGTT
CTAACGTTTTCATGGGTTGGTTTCGCCAGGCACCTGGTAAAGAACGTGGA
TTTGTGGCATCCATTACCCCTCATCACACGGTCACCGACTATGCCGATTC
TGTTCGTGGTCGCTTCAGCATTTCAGCAGATTCTGCGAAGAATACCGTGT
ACTTACAGATGAACTCACTGAAACCTGAGGATACCGCCGTTTATTATTGT
GCGGCAGCCATTTATAGGAACGCGTCGGTTTTGGACTATTGGGGTCAGGG
CACACAAGTCACGGTCTCAAGCGGCGGAAGCTCTGGTGAACAGAAACTGA
TTTCAGAAG
AFGSNVF#PHHTV#AIYRNASVL#evqlvesggglvqagdslrlscvasg
AFGSNVFmgwfrqapgkerefvasitPHHTVtdyadsvrgrfsisadsak
ntvylqmnslkpedtavyycaaAIYRNASVLdywgqgtqvtvss#AAGGA
GATATACCATGGAAGTTCAACTGGTTGAATCTGGTGGCGGTTTAGTTCAA
GCCGGTGATAGCCTTCGTCTGAGCTGCGTCGCAAGCGGAGCTTTCGGTTC
TAACGTTTTCATGGGTTGGTTTCGCCAGGCACCTGGTAAAGAACGTGAAT
TTGTGGCATCCATTACCCCTCATCACACGGTCACCGACTATGCCGATTCT
GTTCGTGGTCGCTTCAGCATTTCAGCAGATTCTGCGAAGAATACCGTGTA
CTTACAGATGAACTCACTGAAACCTGAGGATACCGCCGTTTATTATTGTG
CGGCAGCCATTTATAGGAACGCGTCGGTTTTGGACTATTGGGGTCAGGGC
ACACAAGTCACGGTCTCAAGCGGCGGAAGCTCTGGTGAACAGAAACTGAT
TTCAGAAG
AFGSNVL#PHHTV#AIYRNASVL#evqlvesggglvqagdslrlscaasg
AFGSNVLmgwfrqapgkerefvasitPHHTVtdyadsvrgrfsisadsak
ntvylqmnslkpedtavyycaaAIYRNASVLdywgqgtqvtvss#ATATA
CCATGGAAGTTCAACTGGTTGAATCTGGTGGCGGTTTAGTTCAAGCCGGT
GATAGCCTTCGTCTGAGCTGCGCCGCAAGCGGAGCTTTCGGTTCTAACGT
TTTAATGGGTTGGTTTCGCCAGGCACCTGGTAAAGAACGTGAATTTGTGG
CATCCATTACCCCTCATCACACGGTCACCGACTATGCCGATTCTGTTCGT
GGTCGCTTCAGCATTTCAGCAGATTCTGCGAAGAATACCGTGTACTTACA
GATGAACTCACTGAAACCTGAGGATACCGCCGTTTATTATTGTGCGGCAG
CCATTTATAGGAACGCGTCGGTTTTGGACTATTGGGGTCAGGGCACACAA
GTCACGGTCTCAAGCGGCGGAAGCTCTTGTGAACAGAAACTGATTTCAGA
AG
AFGSNVF#PHHTV#AIYRNASVL#evqlvesggglvqagdslrlscpasg
AFGSNVFmgwfrqapgkerefvasitPHHTVtdyadsvrgrfsisadsak
ntvylqmnslkpedtavyycaaAIYRNASVLdywgqgtqvtvss#ATATA
CCATGGAAGTTCAACTGGTTGAATCTGGTGGCGGTTTAGTTCAAGCCGGT
GATAGCCTTCGTCTGAGCTGCCCCGCAAGCGGAGCTTTCGGTTCTAACGT
TTTCATGGGTTGGTTTCGCCAGGCACCTGGTAAAGAACGTGAATTTGTGG
CATCCATTACCCCTCATCACACGGTCACCGACTATGCCGATTCTGTTCGT
GGTCGCTTCAGCATTTCAGCAGATTCTGCGAAGAATACCGTGTACTTACA
GATGAACTCACTGAAACCTGAGGATACCGCCGTTTATTATTGTGCGGCAG
CCATTTATAGGAACGCGTCGGTTTTGGACTATTGGGGTCAGGGCACACAA
GTCACGGTCTCAAGCGGCGGAAGCTCTGGTGAACAGAAACTGATTTCAGA
AG
AFGSNVF#PHHTV#AIYRNASVL#evqlvesggglvqagdslrlscaasg
AFGSNVFmgwfrqapgkerefvafitPHHTVtdyadsvrgrfsisadsak
ntvylqmnslkpedtavyycaaAIYRNASVLdywgqgtqvtvss#ACCAT
GGAAGTTCAACTGGTTGAATCTGGTGGCGGTTTAGTTCAAGCCGGTGATA
GCCTTCGTCTGAGCTGCGCCGCAAGCGGAGCTTTCGGTTCTAACGTTTTC
ATGGGTTGGTTTCGCCAGGCACCTGGTAAAGAACGTGAATTTGTGGCATT
CATTACCCCTCATCACACGGTCACCGACTATGCCGATTCTGTTCGTGGTC
GCTTCAGCATTTCAGCAGATTCTGCGAAGAATACCGTGTACTTACAGATG
AACTCACTGAAACCTGAGGATACCGCCGTTTATTATTGTGCGGCAGCCAT
TTATAGGAACGCGTCGGTTTTGGACTATTGGGGTCAGGGCACACAAGTCA
CGGTCTCAAGCGGCGGAAGCTCTGGTGAACAGAAACTGATTTCAGAAG
AFGSNVF#PHHTV#AIYRNASVL#evqlvesggglvqagdslrlscaasg
AFGSNVFmgwfrqapgkerefvasitPHHTVtdyadsvrgrfsisedsak
ntvylqmnslkpedtavyycaaAIYRNASVLdywgqgtqvtvss#AGGAG
ATATACCATGGAAGTTCAACTGGTTGAATCTGGTGGCGGTTTAGTTCAAG
CCGGTGATAGCCTTCGTCTGAGCTGCGCCGCAAGCGGAGCTTTCGGTTCT
AACGTTTTCATGGGTTGGTTTCGCCAGGCACCTGGTAAAGAACGTGAATT
TGTGGCATCCATTACCCCTCATCACACGGTCACCGACTATGCCGATTCTG
TTCGTGGTCGCTTCAGCATTTCAGAAGATTCTGCGAAGAATACCGTGTAC
TTACAGATGAACTCACTGAAACCTGAGGATACCGCCGTTTATTATTGTGC
GGCAGCCATTTATAGGAACGCGTCGGTTTTGGACTATTGGGGTCAGGGCA
CACAAGTCACGGTCTCAAGCGGCGGAAGCTCTGGTGAACAGAAACTGATT
TCAGAAG
AFGSNVF#PHHTV#AIYRNASVL#evqlvesggglvhagdrlrlscaasg
AFGSNVFmgwfrqapgkerefvasitPHHTVtdyadsvrgrfsisadsak
ntvylqmnslkpedtavyycaaAIYRNASVLdywgqgtqvtvss#GAAGG
AGATATACCATGGAAGTTCAACTGGTTGAATCTGGTGGCGGTTTAGTTCA
CGCCGGTGATCGCCTTCGTCTGAGCTGCGCCGCAAGCGGAGCTTTCGGTT
CTAACGTTTTCATGGGTTGGTTTCGCCAGGCACCTGGTAAAGAACGTGAA
TTTGTGGCATCCATTACCCCTCATCACACGGTCACCGACTATGCCGATTC
TGTTCGTGGTCGCTTCAGCATTTCAGCAGATTCTGCGAAGAATACCGTGT
ACTTACAGATGAACTCACTGAAACCTGAGGATACCGCCGTTTATTATTGT
GCGGCAGCCATTTATAGGAACGCGTCGGTTTTGGACTATTGGGGTCAGGG
CACACAAGTCACGGTCTCAAGCGGCGGAAGCTCTGGTGAACAGAAACTGA
TTTCAGAAG
AFGSNVF#PHHTV#AIYRNASVF#evqlvesggglvqagdslrlscaasg
AFGSNVFmgwfrqapgkerefvasitPHHTVtdyadsvrgrfsisadsak
ntvylqmnslkpedtavyycaaAIYRNASVFaywgqgtqvtvss#ATACC
ATGGAAGTTCAACTGGTTGAATCTGGTGGCGGTTTAGTTCAAGCCGGTGA
TAGCCTTCGTCTGAGCTGCGCCGCAAGCGGAGCTTTCGGTTCTAACGTTT
TCATGGGTTGGTTTCGCCAGGCACCTGGTAAAGAACGTGAATTTGTGGCA
TCCATTACCCCTCATCACACGGTCACCGACTATGCCGATTCTGTTCGTGG
TCGCTTCAGCATTTCAGCAGATTCTGCGAAGAATACCGTGTACTTACAGA
TGAACTCACTGAAACCTGAGGATACCGCCGTTTATTATTGTGCGGCAGCC
ATTTATAGGAACGCGTCGGTTTTTGCCTATTGGGGTCAGGGCACACAAGT
CACGGTCTCAAGCGGCGGAAGCTCTGGTGAACAGAAACTGATTTCAGAAG
AFGSNVF#PHHTV#AIYRNASVL#evqlvesggglvkvgdslrlscaasg
AFGSNVFmgwfrqapgkerefvasitPHHTVtdyadsvrgrfsisadsak
ntvylqmnslkpedtavyycaaAIYRNASVLdywgqgtqvtvss#ACCAT
GGAAGTTCAACTGGTTGAATCTGGTGGCGGTTTAGTTAAAGTCGGTGATA
GCCTTCGTCTGAGCTGCGCCGCAAGCGGAGCTTTCGGTTCTAACGTTTTC
ATGGGTTGGTTTCGCCAGGCACCTGGTAAAGAACGTGAATTTGTGGCATC
CATTACCCCTCATCACACGGTCACCGACTATGCCGATTCTGTTCGTGGTC
GCTTCAGCATTTCAGCAGATTCTGCGAAGAATACCGTGTACTTACAGATG
AACTCACTGAAACCTGAGGATACCGCCGTTTATTATTGTGCGGCAGCCAT
TTATAGGAACGCGTCGGTTTTGGACTATTGGGGTCAGGGCACACAAGTCA
CGGTCTCAAGCGGCGGAAGCTCTGGTGAACAGAAACTGATTTCAGAAG
AFGSNVF#PHHTV#AIYRNASVL#eiqlvesggglvqagdslrlscaasg
AFGSNVFmgwfrqapgkerefvasitPHHTVtdyadsvrgrfsisadsak
ntvylqmnslkpedtavyycaaAIYRNASVLdywgqgtqvtvss#ATACC
ATGGAAATTCAACTGGTTGAATCTGGTGGCGGTTTAGTTCAAGCCGGTGA
TAGCCTTCGTCTGAGCTGCGCCGCAAGCGGAGCTTTCGGTTCTAACGTTT
TCATGGGTTGGTTTCGCCAGGCACCTGGTAAAGAACGTGAATTTGTGGCA
TCCATTACCCCTCATCACACGGTCACCGACTATGCCGATTCTGTTCGTGG
TCGCTTCAGCATTTCAGCAGATTCTGCGAAGAATACCGTGTACTTACAGA
TGAACTCACTGAAACCTGAGGATACCGCCGTTTATTATTGTGCGGCAGCC
ATTTATAGGAACGCGTCGGTTTTGGACTATTGGGGTCAGGGCACACAAGT
CACGGTCTCAAGCGGCGGAAGCTCTGGTGAACAGAAACTGATTTCAGAAG
AFGSNVF#PHHTV#AIYRNASVL#evqlvesggglvhagdslrlscaasg
AFGSNVFmgwfrqapgkerefvasitPHHTVtdyadsvrgrfsisadsak
ntvylqmnslkpedtavyycaaAIYRNASVLdywgqgtqvtvss#GAAGG
AGATATACCATGGAAGTTCAACTGGTTGAATCTGGTGGCGGTTTAGTTCA
CGCCGGTGATAGCCTTCGTCTGAGCTGCGCCGCAAGCGGAGCTTTCGGTT
CTAACGTTTTCATGGGTTGGTTTCGCCAGGCACCTGGTAAAGAACGTGAA
TTTGTGGCATCCATTACCCCTCATCACACGGTCACCGACTATGCCGATTC
TGTTCGTGGTCGCTTCAGCATTTCAGCAGATTCTGCGAAGAATACCGTGT
ACTTACAGATGAACTCACTGAAACCTGAGGATACCGCCGTTTATTATTGT
GCGGCAGCCATTTATAGGAACGCGTCGGTTTTGGACTATTGGGGTCAGGG
CACACAAGTCACGGTCTCAAGCGGCGGAAGCTCTGGTGAACAGAAACTGA
TTTCAGAAGCCAA
AFGSNVF#PHHTV#AIYRNASVL#evqlvesggglvqagdslrlscaasg
AFGSNVFmgwfrqapgkerefvasitPHHTVtdyadsvrgrfsisadsak
ntvylqmnslkpedtavyycaaAIYRNASVLdywgqgtqgtvss#ACCAT
GGAAGTTCAACTGGTTGAATCTGGTGGCGGTTTAGTTCAAGCCGGTGATA
GCCTTCGTCTGAGCTGCGCCGCAAGCGGAGCTTTCGGTTCTAACGTTTTC
ATGGGTTGGTTTCGCCAGGCACCTGGTAAAGAACGTGAATTTGTGGCATC
CATTACCCCTCATCACACGGTCACCGACTATGCCGATTCTGTTCGTGGTC
GCTTCAGCATTTCAGCAGATTCTGCGAAGAATACCGTGTACTTACAGATG
AACTCACTGAAACCTGAGGATACCGCCGTTTATTATTGTGCGGCAGCCAT
TTATAGGAACGCGTCGGTTTTGGACTATTGGGGTCAGGGCACACAAGGCA
CGGTCTCAAGTGGCGGAAGCTCTGGTGAACAGAAACTGATTTCAGAAGCC
AA
AFGSNVF#PHHTV#AIYRNASVL#evqlvesggslvqagdslrlscaasg
AFGSNVFmgwfrqapgkerefvasitPHHTVtdyadsvrgrfsisadsak
ntvylqmnslkpedtavyycaaAIYRNASVLdywgqgtqvtvss#AAGGA
GATATACCATGGAAGTTCAACTGGTTGAATCTGGTGGCAGTTTAGTTCAA
GCCGGTGATAGCCTTCGTCTGAGCTGCGCCGCAAGCGGAGCTTTCGGTTC
TAACGTTTTCATGGGTTGGTTTCGCCAGGCACCTGGTAAAGAACGTGAAT
TTGTGGCATCCATTACCCCTCATCACACGGTCACCGACTATGCCGATTCT
GTTCGTGGTCGCTTCAGCATTTCAGCAGATTCTGCGAAGAATACCGTGTA
CTTACAGATGAACTCACTGAAACCTGAGGATACCGCCGTTTATTATTGTG
CGGCAGCCATTTATAGGAACGCGTCGGTTTTGGACTATTGGGGTCAGGGC
ACACAAGTCACGGTCTCAAGCGGCGGAAGCTCTGGTGAACAGAAACTGAT
TTCAGAAGCCAA
AFGSKVF#PHHTV#AIYRNASVL#evqlvesggglvqagdslrlscaasg
AFGSKVFmgwfrqapgkerefvasitPHHTVtdyadsvrgrfsisadsak
ntvylqmnslkpedtavyycaaAIYRNASVLdywgqgtqvtvss#GGAGA
TATACCATGGAAGTTCAACTGGTTGAATCTGGTGGCGGTTTAGTTCAAGC
CGGTGATAGCCTTCGTCTGAGCTGCGCCGCAAGCGGAGCTTTCGGTTCTA
AAGTTTTCATGGGTTGGTTTCGCCAGGCACCTGGTAAAGAACGTGAATTT
GTGGCATCCATTACCCCTCATCACACGGTCACCGACTATGCCGATTCTGT
TCGTGGTCGCTTCAGCATTTCAGCAGATTCTGCGAAGAATACCGTGTACT
TACAGATGAACTCACTGAAACCTGAGGATACCGCCGTTTATTATTGTGCG
GCAGCCATTTATAGGAACGCGTCGGTTTTGGACTATTGGGGTCAGGGCAC
ACAAGTCACGGTCTCAAGCGGCGGAAGCTCTGGTGAACAGAAACTGATTT
CAGAAGCCAA
AFGSNVF#PHHTV#AIYRNASVL#evqlvesggglvqagdslrlscatsg
AFGSNVFmgwfrqapgkerefvasitPHHTVtdyadsvrgrfsisadsak
ntvylqmkslkpedtavyycaaAIYRNASVLdywgqgtqvtvss#AGATA
TACCATGGAAGTTCAACTGGTTGAATCTGGTGGCGGTTTAGTTCAAGCCG
GTGATAGCCTTCGTCTGAGCTGCGCCACAAGCGGAGCTTTCGGTTCTAAC
GTTTTCATGGGTTGGTTTCGCCAGGCACCTGGTAAAGAACGTGAATTTGT
GGCATCCATTACCCCTCATCACACGGTCACCGACTATGCCGATTCTGTTC
GTGGTCGCTTCAGCATTTCAGCAGATTCTGCGAAGAATACCGTGTACTTA
CAGATGAAATCACTGAAACCTGAGGATACCGCCGTTTATTATTGTGCGGC
AGCCATTTATAGGAACGCGTCGGTTTTGGACTATTGGGGGCAGGGCACAC
AAGTCACGGTCTCAAGCGGCGGAAGCTCTGGTGAACAGAAACTGATTTCA
GAAGCCAA
AFGSNVF#PHHTV#VIYRNASVL#evqlvesggglvqagdslrlscaasg
AFGSNVFmgwfrqapgkerefvasitPHHTVtdyadsvrgrfsisadsak
ntvylqmnslkpedtavyycaaVIYRNASVLdywgqgtqvtvss#AAGGA
GATATACCATGGAAGTTCAACTGGTTGAATCTGGTGGCGGTTTAGTTCAA
GCCGGTGATAGCCTTCGTCTGAGCTGCGCCGCAAGCGGAGCTTTCGGTTC
TAACGTTTTCATGGGTTGGTTTCGCCAGGCACCTGGTAAAGAACGTGAAT
TTGTGGCATCCATTACCCCTCATCACACGGTCACCGACTATGCCGATTCT
GTTCGTGGTCGCTTCAGCATTTCAGCAGATTCTGCGAAGAATACCGTGTA
CTTACAGATGAACTCACTGAAACCTGAGGATACCGCCGTTTATTATTGTG
CGGCAGTCATTTATAGGAACGCGTCGGTTTTGGACTATTGGGGTCAGGGC
ACACAAGTCACGGTCTCAAGCGGCGGAAGCTCTGGTGAACAGAAACTGAT
TTCAGAAGCCAA
AFGSNVF#PHHTV#AIYRNASVL#evqlvesggglvqagdslrlscaasg
AFGSNVFmgwfrqapgkerefvasltPHHTVtdyadsvrgrfsisadsak
ntvylqmnslkpedtavyycaaAIYRNASVLdywgqgtqvtvss#AGGAG
ATATACCATGGAAGTTCAACTGGTTGAATCTGGTGGCGGTTTAGTTCAAG
CCGGTGATAGCCTTCGTCTGAGCTGCGCCGCAAGCGGAGCTTTCGGTTCT
AACGTTTTCATGGGTTGGTTTCGCCAGGCACCTGGTAAAGAACGTGAATT
TGTGGCATCCCTTACCCCTCATCACACGGTCACCGACTATGCCGATTCTG
TTCGTGGTCGCTTCAGCATTTCAGCAGATTCTGCGAAGAATACCGTGTAC
TTACAGATGAACTCACTGAAACCTGAGGATACCGCCGTTTATTATTGTGC
GGCAGCCATTTATAGGAACGCGTCGGTTTTGGACTATTGGGGTCAGGGCA
CACAAGTCACGGTCTCAAGCGGCGGAAGCTCTGGTGAACAGAAACTGATT
TCAGAAGCCAA
AFGSNVF#PHHTV#AIYRNASVL#evqlvdsggglvqagdrlrlscaasg
AFGSNVFmgwfrqapgkerefvasitPHHTVtdyadsvrgrfsisadsak
ntvylqmnslkpedtavyycaaAIYRNASVLdywgqgtqgtvss#GAAGG
AGATATACCATGGAAGTTCAACTGGTTGACTCTGGTGGCGGTTTAGTTCA
AGCCGGTGATCGCCTTCGTCTGAGCTGCGCCGCAAGCGGAGCTTTCGGTT
CTAACGTTTTCATGGGTTGGTTTCGCCAGGCACCTGGTAAAGAACGTGAA
TTTGTGGCATCCATTACCCCTCATCACACGGTCACCGACTATGCCGATTC
TGTTCGTGGTCGCTTCAGCATTTCAGCAGATTCTGCGAAGAATACCGTGT
ACTTACAGATGAACTCACTGAAACCTGAGGATACCGCCGTTTATTATTGT
GCGGCAGCCATTTATAGGAACGCGTCGGTTTTGGACTATTGGGGTCAGGG
CACACAAGGCACGGTCTCAAGCGGCGGAAGCTCTGGTGAACAGAAACTGA
TTTCAGAAGCCAA
AFGSNVF#PHHTV#AIYRNASVL#evqlvesggglvqagdslrlscaasg
AFGSNVFmgwfrqapgkerefvasitPHHTVtdyadsvrgrfsisadsak
ntvylqmnslkpedtavyycatAIYRNASVLdywgqgtqvtvss#GGAGA
TATACCATGGAAGTTCAACTGGTTGAATCTGGTGGCGGTTTAGTTCAAGC
CGGTGATAGCCTTCGTCTGAGCTGCGCCGCAAGCGGAGCTTTCGGTTCTA
ACGTTTTCATGGGTTGGTTTCGCCAGGCACCTGGTAAAGAACGTGAATTT
GTGGCATCCATTACCCCTCATCACACGGTCACCGACTATGCCGATTCTGT
TCGTGGTCGCTTCAGCATTTCAGCAGATTCTGCGAAGAATACCGTGTACT
TACAGATGAACTCACTGAAACCTGAGGATACCGCCGTTTATTATTGTGCG
ACAGCCATTTATAGGAACGCGTCGGTTTTGGACTATTGGGGTCAGGGCAC
ACAAGTCACGGTCTCAAGCGGCGGAAGCTCTGGTGAACAGAAACTGATTT
CAGAAGCCAA
AFGSNVF#PHHTV#AIYRNASVL#evqlvesggglvqagdslrlscaasg
AFGSNVFmgcfrqapgkerefvasitPHHTVtdyadsvrgrfsisadsak
ntvylqmnslkpedtavyycaaAIYRNASVLdywgqgtqgtvss#ACCAT
GGAAGTTCAACTGGTTGAATCTGGTGGCGGTTTAGTTCAAGCCGGTGATA
GCCTTCGTCTGAGCTGCGCCGCAAGCGGAGCTTTCGGTTCTAACGTTTTC
ATGGGTTGTTTTCGCCAGGCACCTGGTAAAGAACGTGAATTTGTGGCATC
CATTACCCCTCATCACACGGTCACCGACTATGCCGATTCTGTTCGTGGTC
GCTTCAGCATTTCAGCAGATTCTGCGAAGAATACCGTGTACTTACAGATG
AACTCACTGAAACCTGAGGATACCGCCGTTTATTATTGTGCGGCAGCCAT
TTATAGGAACGCGTCGGTTTTGGACTATTGGGGTCAGGGCACACAAGGCA
CGGTCTCAAGCGGCGGAAGCTCTGGTGAACAGAAACTGATTTCAGAAGCC
AA
AFGSNVF#PHHTV#AIYGNASVL#evqlvesggglvqagdslrlscaasg
AFGSNVFmgwfrqapgkerefvasitPHHTVtdyadsvrgrfsisadsak
ntvylqmnslkpedtavyycaaAIYGNASVLdywgqgtqvtvss#AGATA
TACCATGGAAGTTCAACTGGTTGAATCTGGTGGCGGTTTAGTTCAAGCCG
GTGATAGCCTTCGTCTGAGCTGCGCCGCAAGCGGAGCTTTCGGTTCTAAC
GTTTTCATGGGTTGGTTTCGCCAGGCACCTGGTAAAGAACGTGAATTTGT
GGCATCCATTACCCCTCATCACACGGTCACCGACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGGC
AGCCATTTATGGGAACGCGTCGGTTTTGGACTATTGGGGTCAGGGCACAC
AAGTCACGGTCTCAAGCGGCGGAAGCTCTGGTGAACAGAAACTGATTTCA
GAAGCCAA
DFGYNVF#PHHTV#AIYRNASVL#evqlvesggglvqagdslrlscaesg
DFGYNVFmgwfrqapgkerefvasitPHHTVtdyadsvrgrfsisadsak
ntvylqmnslkpedtavyycaaAIYRNASVLdywghgtqvtvss#ACCAT
GGAAGTTCAACTGGTTGAATCTGGTGGCGGTTTAGTTCAAGCCGGAGATA
GCCTTCGTCTGAGCTGCGCCGAAAGCGGAGATTTCGGTTATAACGTTTTC
ATGGGTTGGTTTCGCCAAGCACCTGGTAAAGAACGTGAATTTGTGGCATC
CATTACCCCTCATCACACGGTCACCGACTATGCCGATTCTGTTCGTGGTC
GCTTCAGCATTTCAGCAGATTCTGCGAAGAATACCGTGTACTTACAGATG
AACTCACTGAAACCTGAGGATACCGCCGTTTATTATTGTGCGGCAGCCAT
TTATAGGAACGCGTCGGTTTTGGACTATTGGGGTCATGGCACACAAGTCA
CGGTCTCAAGCGGCGGAAGCTCTGGTGAACAGAAACTTATTTCAGAAGCC
AA
AFGSNVF#PHHTV#AIYRNASVL#evqlfesggglvqagdslrlscaasg
AFGSNVFmgwfrqapgkerefvasitPHHTVtdyadsvrgrfsfsadsak
ntvylqmnslkpedtavyycaaAIYRNASVLdywgqgtqvtvss#AGATA
TACCATGGAAGTTCAACTGTTTGAATCTGGTGGCGGTTTAGTTCAAGCCG
GTGATAGCCTTCGTCTGAGCTGCGCCGCAAGCGGAGCTTTCGGTTCTAAC
GTTTTCATGGGTTGGTTTCGCCAGGCACCTGGTAAAGAACGTGAATTTGT
GGCATCCATTACCCCTCATCACACGGTCACCGACTATGCCGATTCTGTTC
GTGGTCGCTTCAGCTTTTCAGCAGATTCTGCGAAGAATACCGTGTACTTA
CAGATGAACTCACTGAAACCTGAGGATACCGCCGTTTATTATTGTGCGGC
AGCCATTTATAGGAACGCGTCGGTTTTGGACTATTGGGGTCAGGGCACAC
AAGTCACGGTCTCAAGCGGCGGAAGCTCTGGTGAACAGAAACTGATTTCA
GAAGCCAA

TABLE 3
Sequences belonging to cluster SR4.
Each line in the table represents one sequence,
both segments and full length of the sequence
are shown. Shown items are divided by “#” and
in the order from start to end of each line is:
CDR1 amino acid sequence, CDR2 amino acid
sequence, CDR3 amino acid sequence, full-length
amino acid sequence with CDRs capitalized,
full-length DNA sequence.
(SEQ ID NOS: 401-690)

FQLSCYC#QHAFR#GEHCLAF#qvqlqesggvlvqagdslrlscaasgFQ
LSCYCmgwfrqapgkerefvasitQHAFRtyyadsvkgrftisrddarnt
vylqmnslkpedtavyycaaGEHCLAFdywgqgtqvtvss#ATATACCAT
GCAGGTTCAACTGCAAGAATCTGGTGGCGTTTTAGTTCAAGCCGGTGATA
GCCTTCGTCTGAGCTGCGCCGCAAGCGGATTTCAGCTCTCGTGTTACTGT
ATGGGTTGGTTTCGCCAGGCACCTGGTAAAGAACGTGAATTTGTGGCATC
CATTACCCAGCACGCCTTTCGTACCTACTATGCCGATTCTGTTAAAGGTC
GCTTCACTATTTCACGCGATGATGCGCGTAATACCGTGTACTTACAGATG
AACTCACTGAAACCTGAGGATACCGCCGTTTATTATTGTGCGGCAGGCGA
GCACTGTCTCGCCTTCGACTATTGGGGTCAGGGCACACAAGTCACGGTCT
CAAGCGGCGGAAGCTCTGGTGAACAGAAACTGATTTCAGAAG
FQ
LSCYC#QHAFR#AETPNILVYE#evqlvesggglvqagdslrlscaasgF
QLSCYCmgwfrqapgkerefvasitQHAFRtyyadsvkgrftisrddarn
tvylqmnslkpedtavyycaaAETPNILVYEnywgqgtqvtvss#ACCAT
GGAAGTTCAACTGGTTGAATCTGGTGGCGGTTTAGTTCAAGCCGGTGATA
GCCTTCGTCTGAGCTGCGCCGCAAGCGGATTTCAGCTCTCGTGTTACTGT
ATGGGTTGGTTTCGCCAGGCACCTGGTAAAGAACGTGAATTTGTGGCATC
CATTACCCAGCACGCCTTTCGTACCTACTATGCCGATTCTGTTAAAGGTC
GCTTCACTATTTCACGCGATGATGCGCGTAATACCGTGTACTTACAGATG
AACTCACTGAAACCTGAGGATACCGCCGTTTATTATTGTGCGGCAGCCGA
GACCCCGAATATCCTCGTGTACGAGAACTATTGGGGTCAGGGCACACAAG
TCACGGTCTCAAGCGGCGGAAGCTCTGGTGAACAGAAACTGATTTCAGAA
G
FQLSCYC#QHAFR#GEHCLAF#qvqlqesggvlvqagdslrls
caasgFQLSCYCmgwfrqapgkerefvasitQHAFRtyyadsvkgrftis
rddarntvylqmnslkpkdtavyycaaGEHCLAFdywgqgtqvtvss#AT
ATACCATGCAGGTTCAACTGCAAGAATCTGGTGGCGTTTTAGTTCAAGCC
GGTGATAGCCTTCGTCTGAGCTGCGCCGCAAGCGGATTTCAGCTCTCGTG
TTACTGTATGGGTTGGTTTCGCCAGGCACCTGGTAAAGAACGTGAATTTG
TGGCATCCATTACCCAGCACGCCTTTCGTACCTACTATGCCGATTCTGTT
AAAGGTCGCTTCACTATTTCACGCGATGATGCGCGTAATACCGTGTACTT
ACAGATGAACTCACTGAAACCTAAGGATACCGCCGTTTATTATTGTGCGG
CAGGCGAGCACTGTCTCGCCTTCGACTATTGGGGTCAGGGCACACAAGTC
ACGGTCTCAAGCGGCGGAAGCTCTGGTGAACAGAAACTGATTTCAGAAG
FQLSCYC#QHAFR#DPSFSDAIAS#evqlvesggglvqagdslrl
scaasgFQLSCYCmgwfrqapgkerefvasitQHAFRtyyadsvkgrfti
srddarntvylqtnslkpedtavyycavDPSFSDAIASdywgqgtqvtvs
s#AAGGAGATATACCATGGAAGTTCAACTGGTTGAATCTGGTGGCGGTTT
AGTTCAAGCCGGTGATAGCCTTCGTCTGAGCTGCGCCGCAAGCGGATTTC
AGCTCTCGTGTTACTGTATGGGTTGGTTTCGCCAGGCACCTGGTAAAGAA
CGTGAATTTGTGGCATCCATTACCCAGCACGCCTTTCGTACCTACTATGC
CGATTCTGTTAAAGGTCGCTTCACTATTTCACGCGATGATGCGCGTAATA
CCGTGTACTTACAGACGAACTCACTGAAACCTGAGGATACCGCCGTTTAT
TATTGTGCGGTTGATCCTTCTTTCTCCGACGCTATCGCCTCGGACTATTG
GGGTCAGGGCACACAAGTCACGGTCTCAAGCGGCGGAAGCTCTGGTGAAC
AGAAACTGATTTCAGAAG
FQLSCYC#QHAFR#GEHCLAF#qvql
qesggglvqagdslrlscaasgFQLSCYCmgwfrqapgkerefvasitQH
AFRtyyadsvkgrftisrddarntvylqmnslkpedtavyycaaGEHCLA
Fdywgqgtqvtvss#ACCATGCAGGTTCAACTGCAAGAATCTGGTGGCGG
TTTAGTTCAAGCCGGTGATAGCCTTCGTCTGAGCTGCGCCGCAAGCGGAT
TTCAGCTCTCGTGTTACTGTATGGGTTGGTTTCGCCAGGCACCTGGTAAA
GAACGTGAATTTGTGGCATCCATTACCCAGCACGCCTTTCGTACCTACTA
TGCCGATTCTGTTAAAGGTCGCTTCACTATTTCACGCGATGATGCGCGTA
ATACCGTGTACTTACAGATGAACTCACTGAAACCTGAGGATACCGCCGTT
TATTATTGTGCGGCAGGCGAGCACTGTCTCGCCTTCGACTATTGGGGTCA
GGGCACACAAGTCACGGTCTCAAGCGGCGGAAGCTCTGGTGAACAGAAAC
TGATTTCAGAAG
FQLSCYC#QHAFR#AETPNILVYE#evqlves
ggglvqagdslrlscaasgFQLSCYCmgwfrqapgkerefvasitQHAFR
tyyadsvkgrftisrddarntvylqmnslkpedtavyycaaAETPNILVY
Etywgqgtqvtvss#AAGGAGATATACCATGGAAGTTCAACTGGTTGAAT
CTGGTGGCGGTTTAGTTCAAGCCGGTGATAGCCTTCGTCTGAGCTGCGCC
GCAAGCGGATTTCAGCTCTCGTGTTACTGTATGGGTTGGTTTCGCCAGGC
ACCTGGTAAAGAACGTGAATTTGTGGCATCCATTACCCAGCACGCCTTTC
GTACCTACTATGCCGATTCTGTTAAAGGTCGCTTCACTATTTCACGCGAT
GATGCGCGTAATACCGTGTACTTACAGATGAACTCACTGAAACCTGAGGA
TACCGCCGTTTATTATTGTGCGGCAGCCGAGACCCCGAATATCCTCGTGT
ACGAGACCTATTGGGGTCAGGGCACACAAGTCACGGTCTCAAGCGGCGGA
AGCTCTGGTGAACAGAAACTGATTTCAGAAG
FQLSCYC#QHAFRDPSFSDAIAS#evqlvesggglvqagdslrlscaasg
FQLSCYCmgwfr
qapgkerefvasitQHAFRtyyadsvkgrftisrddarntvylqmnslkp
edtavyycavDPSFSDAIASdywgqgtqvtvss#ACCATGGAAGTTCAAC
TGGTTGAATCTGGTGGCGGTTTAGTTCAAGCCGGTGATAGCCTTCGTCTG
AGCTGCGCCGCAAGCGGATTTCAGCTCTCGTGTTACTGTATGGGTTGGTT
TCGCCAGGCACCTGGTAAAGAACGTGAATTTGTGGCATCCATTACCCAGC
ACGCCTTTCGTACCTACTATGCCGATTCTGTTAAAGGTCGCTTCACTATT
TCACGCGATGATGCGCGTAATACCGTGTACTTACAGATGAACTCACTGAA
ACCTGAGGATACTGCCGTTTATTATTGTGCGGTTGATCCTTCTTTCTCCG
ACGCTATCGCCTCGGACTATTGGGGTCAGGGCACACAAGTCACGGTCTCA
AGCGGCGGAAGCTCTGGTGAACAGAAACTGATTTCAGAAG
FQLS
CYC#QHAFR#AETPNILVYE#evqlvesggvlvqagdslrlscaasgFQL
SCYCmgwfrqapgkerefvasitQHAFRtyyadsvkgrftisrddarntv
ylqmnslkpedtavyycaaAETPNILVYEnywgqgtqvtvss#ACCATGG
AAGTTCAACTGGTTGAATCTGGTGGCGTTTTAGTTCAAGCCGGTGATAGC
CTTCGTCTGAGCTGCGCCGCAAGCGGATTTCAGCTCTCGTGTTACTGTAT
GGGTTGGTTTCGCCAGGCACCTGGTAAAGAACGTGAATTTGTGGCATCCA
TTACCCAGCACGCCTTTCGTACCTACTATGCCGATTCTGTTAAAGGTCGC
TTCACTATTTCACGCGATGATGCGCGTAATACCGTGTACTTACAGATGAA
CTCACTGAAACCTGAGGATACCGCCGTTTATTATTGTGCGGCAGCCGAGA
CCCCGAATATCCTCGTGTACGAGAACTATTGGGGTCAGGGCACACAAGTC
ACGGTCTCAAGCGGCGGAAGCTCTGGTGAACAGAAACTGATTTCAGAAG
FQLSCYC#QHAFR#AETPNILVYE#evqlvesggglvqagnslrl
scaasgFQLSCYCmgwfrqapgkerefvasitQHAFRtyyadsvkgrfti
srddarntvylqmnslkpedtavyycaaAETPNILVYEnywgqgtqvtvs
s#ACCATGGAAGTTCAACTGGTTGAATCTGGTGGCGGTTTAGTTCAAGCC
GGTAATAGCCTTCGTCTGAGCTGCGCCGCAAGCGGATTTCAGCTCTCGTG
TTACTGTATGGGTTGGTTTCGCCAGGCACCTGGTAAAGAACGTGAATTTG
TGGCATCCATTACCCAGCACGCCTTTCGTACCTACTATGCCGATTCTGTT
AAAGGTCGCTTCACTATTTCACGCGATGATGCGCGTAATACCGTGTACTT
ACAGATGAACTCACTGAAACCTGAGGATACCGCCGTTTATTATTGTGCGG
CAGCCGAGACCCCGAATATCCTCGTGTACGAGAACTATTGGGGTCAGGGC
ACACAAGTCACGGTCTCAAGCGGCGGAAGCTCTGGTGAACAGAAACTGAT
TTCAGAAG
FQLSCYC#QHAFR#GEHCLAF#qvqlqesggvlvqa
gdslrlscaasgFQLSCYCmgwfrqapgkerefvasitQHAFRtyyadsv
kgrftisrddvrntvylqmnslkpedtavyycaaGEHCLAFdywgqgtqv
tvss#AAGGAGATATACCATGCAGGTTCAACTGCAAGAATCTGGTGGCGT
TTTAGTTCAAGCCGGTGATAGCCTTCGTCTGAGCTGCGCCGCAAGCGGAT
TTCAGCTCTCGTGTTACTGTATGGGTTGGTTTCGCCAGGCACCTGGTAAA
GAACGTGAATTTGTGGCATCCATTACCCAGCACGCCTTTCGTACCTACTA
TGCCGATTCTGTTAAAGGTCGCTTCACTATTTCACGCGATGATGTGCGTA
ATACCGTGTACTTACAGATGAACTCACTGAAACCTGAGGATACCGCCGTT
TATTATTGTGCGGCAGGCGAGCACTGTCTCGCCTTCGACTATTGGGGTCA
GGGCACACAAGTCACGGTCTCAAGCGGCGGAAGCTCTGGTGAACAGAAAC
TGATTTCAGAAG
FQLSCYC#QHAFR#AETPNILVYE#evqlves
ggglvqagdslrlscaasgFQLSCYCmgwfrqapgkerefvasitQHAFR
tyyadsvkgrftisrddarntvylqmnslkpedtavyycaaAETPNILVY
Enyrgqgtqvtvss#ATATACCATGGAAGTTCAACTGGTTGAATCTGGTG
GCGGTTTAGTTCAAGCCGGTGATAGCCTTCGTCTGAGCTGCGCCGCAAGC
GGATTTCAGCTCTCGTGTTACTGTATGGGTTGGTTTCGCCAGGCACCTGG
TAAAGAACGTGAATTTGTGGCATCCATTACCCAGCACGCCTTTCGTACCT
ACTATGCCGATTCTGTTAAAGGTCGCTTCACTATTTCACGCGATGATGCG
CGTAATACCGTGTACTTACAGATGAACTCACTGAAACCTGAGGATACCGC
CGTTTATTATTGTGCGGCAGCCGAGACCCCGAATATCCTCGTGTACGAGA
ACTATCGGGGTCAGGGCACACAAGTCACGGTCTCAAGCGGCGGAAGCTCT
GGTGAACAGAAACTGATTTCAGAAG
FPLSCYC#QHAFR#AETPN
ILVYE#evqlvesggglvqagdslrlscaasgFPLSCYCmgwfrqapgke
refvasitQHAFRtyyadsvkgrftisrddarntvylqmnslkpedtavy
ycaaAETPNILVYEtywgqgtqvtvss#ATACCATGGAAGTTCAACTGGT
TGAATCTGGTGGCGGTTTAGTTCAAGCCGGTGATAGCCTTCGGCTGAGCT
GCGCCGCAAGCGGATTTCCGCTCTCGTGTTACTGTATGGGTTGGTTTCGC
CAGGCACCTGGTAAAGAACGTGAATTTGTGGCATCCATTACCCAGCACGC
CTTTCGTACCTACTATGCCGATTCTGTTAAAGGTCGCTTCACTATTTCAC
GCGATGATGCGCGTAATACCGTGTACTTACAGATGAACTCACTGAAACCT
GAGGATACCGCCGTTTATTATTGTGCGGCAGCCGAGACCCCGAATATCCT
CGTGTACGAGACCTATTGGGGTCAGGGCACACAAGTCACGGTCTCAAGCG
GCGGAAGCTCTGGTGAACAGAAACTGATTTCAGAAG
FQLSCYC#
QHAFR#AETPNILVYE#evqlvesggglvqagdslrmscaasgFQLSCYC
mgwfrqapgkerefvasitQHAFRtyyadsvkgrftisrddarntvylqm
nslkpedtavyycaaAETPNILVYEnywgqgtqvtvss#GGAGATATACC
ATGGAAGTTCAACTGGTTGAATCTGGTGGCGGTTTAGTTCAAGCCGGTGA
TAGCCTTCGTATGAGCTGCGCCGCAAGCGGATTTCAGCTCTCGTGTTACT
GTATGGGTTGGTTTCGCCAGGCACCTGGTAAAGAACGTGAATTTGTGGCA
TCCATTACCCAGCACGCCTTTCGTACCTACTATGCCGATTCTGTTAAAGG
TCGCTTCACTATTTCACGCGATGATGCGCGTAATACCGTGTACTTACAGA
TGAACTCACTGAAACCTGAGGATACCGCCGTTTATTATTGTGCGGCAGCC
GAGACCCCGAATATCCTCGTGTACGAGAACTATTGGGGTCAGGGCACACA
AGTCACGGTCTCAAGCGGCGGAAGCTCTGGTGAACAGAAACTGATTTCAG
AAG
FQLSCYC#QHAFR#AETPNILVYE#evqlvesggglvqagd
slrlscaasgFQLSCYCmgcfrqapgkerefvasitQHAFRtyyadsvkg
rftisrddvrntvylqmnslkpedtavyycaaAETPNILVYEnywgqgtq
vtvss#ACCATGGAAGTTCAACTGGTTGAATCTGGTGGCGGTTTAGTTCA
AGCCGGTGATAGCCTTCGTCTGAGCTGCGCCGCAAGCGGATTTCAGCTCT
CGTGTTACTGTATGGGTTGTTTTCGCCAGGCACCTGGTAAAGAACGTGAA
TTTGTGGCATCCATTACCCAGCACGCCTTTCGTACCTACTATGCCGATTC
TGTTAAAGGTCGCTTCACTATTTCACGCGATGATGTGCGTAATACCGTGT
ACTTACAGATGAACTCACTGAAACCTGAGGATACCGCCGTTTATTATTGT
GCGGCAGCCGAGACCCCGAATATCCTCGTGTACGAGAACTATTGGGGTCA
GGGCACACAAGTCACGGTCTCAAGCGGCGGAAGCTCTGGTGAACAGAAAC
TGATTTCAGAAG
FQLSCYC#QHAFR#DPSFSDAIAS#evqlves
ggglvqagdslrlscaasgFQLSCYCmgwfrqapgkerefvasitQHAFR
tyyadsvkgrftisrddarntvylqmnslkpedtavyycvvGCGGATTTC
AGCTCTCGTGTTACTGTATGGGTTGGTTTCGCCAGGCACCTGGTAAAGAA
CGTGAATTTGTGGCATCCATTACCCAGCACGCCTTTCGTACCTACTATGC
CGATTCTGTTAAAGGTCGCTTCACTATTTCACGCGATGATGCGCGTAATA
CCGTGTACTTACAGATGAACTCACTGAAACCTGAGGATACCGCCGTTTAT
TATTGTGTGGTTGATCCTTCTTTCTCTGACGCCATCGCCTCGGACTATTG
GGGTCAGGGCACACAAGTCACGGTCTCAAGCGGCGGAAGCTCTGGTGAAC
AGAAACTGATTTCAGAAG
FQLSCYC#QHAFR#AETPNILVYE#e
vqlvesggglvqagdslrlscaasgFQLSCYCmgwfrqapgkehefvasi
tQHAFRtyyadsvkgrftisrddarntvylqmnslkpedtavyycaaAET
PNILVYEnywgqgtqvtvss#ATACCATGGAAGTTCAACTGGTTGAATCT
GGTGGCGGTTTAGTTCAAGCCGGTGATAGCCTTCGTCTGAGCTGCGCCGC
AAGCGGATTTCAGCTCTCGTGTTACTGTATGGGTTGGTTTCGCCAGGCAC
CTGGTAAAGAACATGAATTTGTGGCATCCATTACCCAGCACGCCTTTCGT
ACCTACTATGCCGATTCTGTTAAAGGTCGCTTCACTATTTCACGCGATGA
TGCGCGTAATACCGTGTACTTACAGATGAACTCACTGAAACCTGAGGATA
CCGCCGTTTATTATTGTGCGGCAGCCGAGACCCCGAATATCCTCGTGTAC
GAGAACTATTGGGGTCAGGGCACACAAGTCACGGTCTCAAGCGGCGGAAG
CTCTGGTGAACAGAAACTGATTTCAGAAG
FQLSCYC#QHAFR#A
ETPNILVYE#evqlvesggglvqagdslrlscaasgFQLSCYCmgcfrqa
pgkerefvasitQHAFRtyyadsvkgrftisrddarntvylqmnslkped
tavyycaaAETPNILVYEnywgqgtqvtvss#AGATATACCATGGAAGTT
CAACTGGTTGAATCTGGTGGCGGTTTAGTTCAAGCCGGTGATAGCCTTCG
TCTGAGCTGCGCCGCAAGCGGATTTCAGCTCTCGTGTTACTGTATGGGTT
GTTTTCGCCAGGCACCTGGTAAAGAACGTGAATTTGTGGCATCCATTACC
CAGCACGCCTTTCGTACCTACTATGCCGATTCTGTTAAAGGTCGCTTCAC
TATTTCACGCGATGATGCGCGTAATACCGTGTACTTACAGATGAACTCAC
TGAAACCTGAGGATACCGCCGTTTATTATTGTGCGGCAGCCGAGACCCCG
AATATCCTCGTGTACGAGAACTATTGGGGTCAGGGCACACAAGTCACGGT
CTCAAGCGGCGGAAGCTCTGGTGAACAGAAACTGATTTCAGAAG
FQLSCYC#QHAFR#AETPNILVYE#evqlvesggglvqagdslrlscaas
gFQLSCYCmgwfrqapgkerefvasitQHAFRtyyadsvkgrftisrdda
rntvylqmnslkpedtavyycaaAETPNILVYEnywcqgtqvtvss#ACC
ATGGAAGTTCAACTGGTTGAATCTGGTGGCGGTTTAGTTCAAGCCGGTGA
TAGCCTTCGTCTGAGCTGCGCCGCAAGCGGATTTCAGCTCTCGTGTTACT
GTATGGGTTGGTTTCGCCAGGCACCTGGTAAAGAACGTGAATTTGTGGCA
TCCATTACCCAGCACGCCTTTCGTACCTACTATGCCGATTCTGTTAAAGG
TCGCTTCACTATTTCACGCGATGATGCGCGTAATACCGTGTACTTACAGA
TGAACTCACTGAAACCTGAGGATACCGCCGTTTATTATTGTGCGGCAGCC
GAGACCCCGAATATCCTCGTGTACGAGAACTATTGGTGTCAGGGCACACA
AGTCACGGTCTCAAGCGGCGGAAGCTCTGGTGAACAGAAACTGATTTCAG
AAG
FQLSCYC#QHAFR#DPSFSDAIAS#evqlvesggglvqagd
slrlscaasgFQLSCYCmgwfrqapgkerefvasitQHAFRtyyadsvkg
rftisrddarntvylqmnslkpgdtavyycavDPSFSDAIASdywgqgtq
vtvsr#ATATACCATGGAAGTTCAACTGGTTGAATCTGGTGGCGGTTTAG
TTCAAGCCGGTGATAGCCTTCGTCTGAGCTGCGCCGCAAGCGGATTTCAG
CTCTCGTGTTACTGTATGGGTTGGTTTCGCCAGGCACCTGGTAAAGAACG
TGAATTTGTGGCATCCATTACCCAGCACGCCTTTCGTACCTACTATGCCG
ATTCTGTTAAAGGTCGCTTCACTATTTCACGCGATGATGCGCGTAATACC
GTGTACTTACAGATGAACTCACTGAAACCTGGGGATACCGCCGTTTATTA
TTGTGCGGTTGATCCTTCTTTCTCCGACGCTATCGCCTCGGACTATTGGG
GTCAGGGCACACAAGTCACGGTCTCACGCGGCGGAAGCTCTGGTGAACAG
AAACTGATTTCAGAAG
FQLSCYC#QHAFR#DPSFSDAIAS#evq
lvesggglvqagdrlrlscaasgFQLSCYCmgwfrqapgkerefvasitQ
HAFRtyyadsvkgrftisrddarntvylqmnslkpedtavyycavDPSFS
DAIASdywgqgtqvtvss#GGAGATATACCATGGAAGTTCAACTGGTTGA
ATCTGGTGGCGGTTTAGTTCAAGCCGGTGATAGACTTCGTCTGAGCTGCG
CCGCAAGCGGATTTCAGCTCTCGTGTTACTGTATGGGTTGGTTTCGCCAG
GCACCTGGTAAAGAACGTGAATTTGTGGCATCCATTACCCAGCACGCCTT
TCGTACCTACTATGCCGATTCTGTTAAAGGTCGCTTCACTATTTCACGCG
ATGATGCGCGTAATACCGTGTACTTACAGATGAACTCACTAAAACCTGAG
GATACCGCCGTTTATTATTGTGCGGTTGATCCTTCTTTCTCCGACGCTAT
CGCCTCGGACTATTGGGGTCAGGGCACACAAGTCACGGTCTCAAGCGGCG
GAAGCTCTGGTGAACAGAAACTGATTTCAGAAG
FQLSCYC#QHA
FR#AETPNILVYE#egqlvesggglvqagdslrlscaasgFQLSCYCmgw
frqapgkerefvasitQHAFRtyyadsvkgrftisrddarntvylqmnsl
kpedtavyycaaAETPNILVYEnywgqgtqvtvss#ACCATGGAAGGTCA
ACTGGTTGAATCTGGTGGCGGTTTAGTTCAAGCCGGTGATAGCCTTCGTC
TGAGCTGCGCCGCAAGCGGATTTCAGCTCTCGTGTTACTGTATGGGTTGG
TTTCGCCAGGCACCTGGTAAAGAACGTGAATTTGTGGCATCCATTACCCA
GCACGCCTTTCGTACCTACTATGCCGATTCTGTTAAAGGTCGCTTCACTA
TTTCACGCGATGATGCGCGTAATACCGTGTACTTACAGATGAACTCACTG
AAACCTGAGGATACCGCCGTTTATTATTGTGCGGCAGCCGAGACCCCGAA
TATCCTCGTGTACGAGAACTATTGGGGTCAGGGCACACAAGTCACGGTCT
CAAGCGGCGGAAGCTCTGGTGAACAGAAACTGATTTCAGAAG
FQ
LSCYC#QHAFR#AETPNILVYE#evqlvesggglvqagdslrlscaasgF
QLSCYCmgwfrqapgkerefvasitQHAFRtyyadsvkgrftisrddarn
tvylqmnslkpedtavyycaaAETPNILVYEnywgqgtqvtvsr#AAGGA
GATATACCATGGAAGTTCAACTGGTTGAATCTGGTGGCGGTTTAGTTCAA
GCCGGTGATAGCCTTCGTCTGAGCTGCGCCGCAAGCGGATTTCAGCTCTC
GTGTTACTGTATGGGTTGGTTTCGCCAGGCACCTGGTAAAGAACGTGAAT
TTGTGGCATCCATTACCCAGCACGCCTTTCGTACCTACTATGCCGATTCT
GTTAAAGGTCGCTTCACTATTTCACGCGATGATGCGCGTAATACCGTGTA
CTTACAGATGAACTCACTGAAACCTGAGGATACCGCCGTTTATTATTGTG
CGGCAGCCGAGACCCCGAATATCCTCGTGTACGAGAACTATTGGGGTCAG
GGCACACAAGTCACGGTCTCACGCGGCGGAAGCTCTGGTGAACAGAAACT
GATTTCAGAAG
FQLSCYC#QHAFR#AETPNILVYE#evqlvesg
gglvqagdslrlscaasgFQLSCYCigwfrqapgkerefvasitQHAFRt
yyadsvkgrftisrddarntvylqmnslkpedtavyycaaAETPNILVYE
nywgqgtqvtvss#ATACCATGGAAGTTCAACTGGTTGAATCTGGTGGCG
GTTTAGTTCAAGCCGGTGATAGCCTTCGTCTGAGCTGCGCCGCAAGCGGA
TTTCAGCTCTCGTGTTACTGTATAGGTTGGTTTCGCCAGGCACCTGGTAA
AGAACGTGAATTTGTGGCATCCATTACCCAGCACGCCTTTCGTACCTACT
ATGCCGATTCTGTTAAAGGTCGCTTCACTATTTCACGCGATGATGCGCGT
AATACCGTGTACTTACAGATGAACTCACTGAAACCTGAGGATACCGCCGT
TTATTATTGTGCGGCAGCCGAGACCCCGAATATCCTCGTGTACGAGAACT
ATTGGGGTCAGGGCACACAAGTCACGGTCTCAAGCGGCGGAAGCTCTGGT
GAACAGAAACTGATTTCAGAAG
FQLSCSC#QHAFR#AETPNILV
YE#evqlvesggglvqagdslrlscaasgFQLSCSCmgwfrqapgkeref
vasitQHAFRtyyadsvkgrftisrddarntvylqmnslkpedtavyyca
aAETPNILVYEnywgqgtqvtvss#AAGGAGATATACCATGGAAGTTCAA
CTGGTTGAATCTGGTGGCGGTTTAGTTCAAGCCGGTGATAGCCTTCGTCT
GAGCTGCGCCGCAAGCGGATTTCAGCTCTCGTGTTCCTGTATGGGTTGGT
TTCGCCAGGCACCTGGTAAAGAACGTGAATTTGTGGCATCCATTACCCAG
CACGCCTTTCGTACCTACTATGCCGATTCTGTTAAAGGTCGCTTCACTAT
TTCACGCGATGATGCGCGTAATACCGTGTACTTACAGATGAACTCACTGA
AACCTGAGGATACCGCCGTTTATTATTGTGCGGCAGCCGAGACCCCGAAT
ATCCTCGTGTACGAGAACTATTGGGGTCAGGGCACACAAGTCACGGTCTC
AAGCGGCGGAAGCTCTGGTGAACAGAAACTGATTTCAGAAG
FQL
SCYC#QHAFR#AETPNILVYE#evqlvesggglvqagdslrlscaasgFQ
LSCYCmgwfrqapgkerefvasitQHAFRtyyadsvkgrftnsrddarnt
vylqmnslkpedtavyycaaAETPNILVYEnywgqgtqvtvss#AAGGAG
ATATACCATGGAAGTTCAACTGGTTGAATCTGGTGGCGGTTTAGTTCAAG
CCGGTGATAGCCTTCGTCTGAGCTGCGCCGCAAGCGGATTTCAGCTCTCG
TGTTACTGTATGGGTTGGTTTCGCCAGGCACCTGGTAAAGAACGTGAATT
TGTGGCATCCATTACCCAGCACGCCTTTCGTACCTACTATGCCGATTCTG
TTAAAGGTCGCTTCACTAATTCACGCGATGATGCGCGTAATACCGTGTAC
TTACAGATGAACTCACTGAAACCTGAGGATACCGCCGTTTATTATTGTGC
GGCAGCCGAGACCCCGAATATCCTCGTGTACGAGAACTATTGGGGTCAGG
GCACACAAGTCACGGTCTCAAGCGGCGGAAGCTCTGGTGAACAGAAACTG
ATTTCAGAAG
FQLSCYC#QHAFR#DPSFSDAIAS#evqlvdsgg
glvqagdrlrlscaasgFQLSCYCmgwfrqapgkerefvasitQHAFRty
yadsvkgrftisrddarntvylqmnslkpedtavyycavDPSFSDAIASd
ywgqgtqvtvss#GAAGGAGATATACCATGGAAGTTCAACTGGTTGACTC
TGGTGGCGGTTTAGTTCAAGCCGGTGATCGCCTTCGTCTGAGCTGCGCCG
CAAGCGGATTTCAGCTCTCGTGTTACTGTATGGGTTGGTTTCGCCAGGCA
CCTGGTAAAGAACGTGAATTTGTGGCATCCATTACCCAGCACGCCTTTCG
TACCTACTATGCCGATTCTGTTAAAGGTCGCTTCACTATTTCACGCGATG
ATGCGCGTAATACCGTGTACTTACAGATGAACTCACTGAAACCTGAGGAT
ACCGCCGTTTATTATTGTGCGGTTGATCCTTCTTTCTCCGACGCTATCGC
CTCGGACTATTGGGGTCAGGGCACACAAGTCACGGTCTCAAGCGGCGGAA
GCTCTGGTGAACAGAAACTGATTTCAGAAG
FQLSCYC#QHAFR#
AETPNILVYE#evqlfesggglvqagdslrlscaasgFQLSCYCmgwfrq
apgkerefvasitQHAFRtyyadsvkgrftisrddarntvylqmnslkpe
dtavyycaaAETPNILVYEnywgqgtqvtvss#AGATATACCATGGAAGT
TCAACTGTTTGAATCTGGTGGCGGTTTAGTTCAAGCCGGTGATAGCCTTC
GTCTGAGCTGTGCCGCAAGCGGATTTCAGCTCTCGTGTTACTGTATGGGT
TGGTTTCGCCAGGCACCTGGTAAAGAACGTGAATTTGTGGCATCCATTAC
CCAGCACGCCTTTCGTACCTACTATGCCGATTCTGTTAAAGGTCGCTTCA
CTATTTCACGCGATGATGCGCGTAATACCGTGTACTTACAGATGAACTCA
CTGAAACCTGAGGATACCGCCGTTTATTATTGTGCGGCAGCCGAGACCCC
GAATATCCTCGTGTACGAGAACTATTGGGGTCAGGGCACACAAGTCACGG
TCTCAAGCGGCGGAAGCTCTGGTGAACAGAAACTGATTTCAGAAG
FQLSCYC#QHAFR#AETPNILVYE#evqlvesggglvqagdslrlscaa
sgFQLSCYCmgwfrqapgkerefvasitQHAFRtyyadsvkgrftisrdd
acntvylqmnslkpedtavyycaaAETPNILVYEnywgqgtqvtvss#AT
ATACCATGGAAGTTCAACTGGTTGAATCTGGTGGCGGTTTAGTTCAAGCC
GGTGATAGCCTTCGTCTGAGCTGCGCCGCAAGCGGATTTCAGCTCTCGTG
TTACTGTATGGGTTGGTTTCGCCAGGCACCTGGTAAAGAACGTGAATTTG
TGGCATCCATTACCCAGCACGCCTTTCGTACCTACTATGCCGATTCTGTT
AAAGGTCGCTTCACTATTTCACGCGATGATGCGTGTAATACCGTGTACTT
ACAGATGAACTCACTGAAACCTGAGGATACCGCCGTTTATTATTGTGCGG
CAGCCGAGACCCCGAATATCCTCGTGTACGAGAACTATTGGGGTCAGGGC
ACACAAGTCACGGTCTCAAGCGGCGGAAGCTCTGGTGAACAGAAACTGAT
TTCAGAAG
FQLSCYC#QHAFR#AETPNILVYE#evqlvdsgggl
vqagdslrlscaasgFQLSCYCmgwfrqapgkerefvasitQHAFRtyya
dsvkgrftisrddarntvylqmnslkpedtavyycaaAETPNILVYEnyw
gqgtqvtvss#GAAGGAGATATACCATGGAAGTTCAACTGGTTGACTCTG
GTGGCGGTTTAGTTCAAGCCGGTGATAGCCTTCGTCTGAGCTGCGCCGCA
AGCGGATTTCAGCTCTCGTGTTACTGTATGGGTTGGTTTCGCCAGGCACC
TGGTAAAGAACGTGAATTTGTGGCATCCATTACCCAGCACGCCTTTCGTA
CCTACTATGCCGATTCTGTTAAAGGTCGCTTCACTATTTCACGCGATGAT
GCGCGTAATACCGTGTACTTACAGATGAACTCACTGAAACCTGAGGATAC
CGCCGTTTATTATTGTGCGGCAGCCGAGACCCCGAATATCCTCGTGTACG
AGAACTATTGGGGTCAGGGCACACAAGTCACGGTCTCAAGCGGCGGAAGC
TCTGGTGAACAGAAACTGATTTCAGAAG
FQLSCYC#QHAFR#AE
TPNILVYE#evqlvesggglvqagdslrlscvasgFQLSCYCmgwfrqap
gkerefvasitQHAFRtyyadsvkgrftisrddarntvylqmnslkpedt
avyycaaAETPNILVYEnywgqgtqvtvss#GAAGGAGATATACCATGGA
AGTTCAACTGGTTGAATCTGGTGGCGGTTTAGTTCAAGCCGGTGATAGCC
TTCGTCTGAGCTGCGTCGCAAGCGGATTTCAGCTCTCGTGTTACTGTATG
GGTTGGTTTCGCCAGGCACCTGGTAAAGAACGTGAATTTGTGGCATCCAT
TACCCAGCACGCCTTTCGTACCTACTATGCCGATTCTGTTAAAGGTCGCT
TCACTATTTCACGCGATGATGCGCGTAATACCGTGTACTTACAGATGAAC
TCACTGAAACCTGAGGATACCGCCGTTTATTATTGTGCGGCAGCCGAGAC
CCCGAATATCCTCGTGTACGAGAACTATTGGGGTCAGGGCACACAAGTCA
CGGTCTCAAGCGGCGGAAGCTCTGGTGAACAGAAACTGATTTCAGAAG
FQLSCYC#QHAFR#AETPNILVYE#evqlvesggglvqagdslrls
raasgFQLSCYCmgwfrqapgkerefvasitQHAFRtyyadsvkgrftis
rddarntvylqmnslkpedtavyycaaAETPNILVYEnywgqgtqvtvss
AAGGAGATATACCATGGAAGTTCAACTGGTTGAATCTGGTGGCGGTTTA
GTTCAAGCCGGTGATAGCCTTCGTCTGAGCCGCGCCGCAAGCGGATTTCA
GCTCTCGTGTTACTGTATGGGTTGGTTTCGCCAGGCACCTGGTAAAGAAC
GTGAATTTGTGGCATCCATTACCCAGCACGCCTTTCGTACCTACTATGCC
GATTCTGTTAAAGGTCGCTTCACTATTTCACGCGATGATGCGCGTAATAC
CGTGTACTTACAGATGAACTCACTGAAACCTGAGGATACCGCCGTTTATT
ATTGTGCGGCAGCCGAGACCCCGAATATCCTCGTGTACGAGAACTATTGG
GGTCAGGGCACACAAGTCACGGTCTCAAGCGGCGGAAGCTCTGGTGAACA
GAAACTGATTTCAGAAG
FQLSCYC#QHAFR#GEHCLAF#qvqlq
esggvlvqvgdslrlscaasgFQLSCYCmgwfrqapgkerefvasitQHA
FRtyyadsvkgrftisrddarntvylqmnslkpedtavyycaaGEHCLAF
dywgqgtqvtvss#AGGAGATATACCATGCAGGTTCAACTGCAAGAATCT
GGTGGCGTTTTAGTTCAAGTCGGTGATAGCCTTCGTCTGAGCTGCGCCGC
AAGCGGATTTCAGCTCTCGTGTTACTGTATGGGTTGGTTTCGCCAGGCAC
CTGGTAAAGAACGTGAATTTGTGGCATCCATTACCCAGCACGCCTTTCGT
ACCTACTATGCCGATTCTGTTAAAGGTCGCTTCACTATTTCACGCGATGA
TGCGCGTAATACCGTGTACTTACAGATGAACTCACTGAAACCTGAGGATA
CCGCCGTTTATTATTGTGCGGCAGGCGAGCACTGTCTCGCCTTCGACTAT
TGGGGTCAGGGCACACAAGTCACGGTCTCAAGCGGCGGAAGCTCTGGTGA
ACAGAAACTGATTTCAGAAG
FQLSCYC#QHAFR#GEHCLAF#qv
qlqesggvlvhagdslrlscaasgFQLSCYCmgwfrqapgkerefvasit
QHAFRtyyadsvkgrftisrddarntvylqmnslkpedtavyycaaGEHC
LAFdywvqgtqvtvss#GAAGGAGATATACCATGCAGGTTCAACTGCAAG
AATCTGGTGGCGTTTTAGTTCACGCCGGTGATAGCCTTCGTCTGAGCTGC
GCCGCAAGCGGATTTCAGCTCTCGTGTTACTGTATGGGTTGGTTTCGCCA
GGCACCTGGTAAAGAACGTGAATTTGTGGCATCCATTACCCAGCACGCCT
TTCGTACCTACTATGCCGATTCTGTTAAAGGTCGCTTCACTATTTCACGC
GATGATGCGCGTAATACCGTGTACTTACAGATGAACTCACTGAAACCTGA
GGATACCGCCGTTTATTATTGTGCGGCAGGCGAGCACTGTCTCGCCTTCG
ACTATTGGGTTCAGGGCACACAAGTCACGGTCTCAAGCGGCGGAAGCTCT
GGTGAACAGAAACTGATTTCAGAAG
FQLSCYC#QHAFR#AETPN
ILVYE#evqlvesggglvqagdslrlscaasgFQLSCYCmgwfrqapgke
refvasitQHAFRtyyadsvkgrftisrddarntvylqmnslkpedtavy
ycaaAETPNILVYEnywvqgtqvtvss#ATATACCATGGAAGTTCAACTG
GTTGAATCTGGTGGCGGTTTAGTTCAAGCCGGTGATAGCCTTCGTCTGAG
CTGCGCTGCAAGCGGATTTCAGCTCTCGTGTTACTGTATGGGTTGGTTTC
GCCAGGCACCTGGTAAAGAACGTGAATTTGTGGCATCCATTACCCAGCAC
GCCTTTCGTACCTACTATGCCGATTCTGTTAAAGGTCGCTTCACTATTTC
ACGCGATGATGCGCGTAATACCGTGTACTTACAGATGAACTCACTGAAAC
CTGAGGATACCGCCGTTTATTATTGTGCGGCAGCCGAGACCCCGAATATC
CTCGTGTACGAGAACTATTGGGTTCAGGGCACACAAGTCACGGTCTCAAG
CGGCGGAAGCTCTTGTGAACAGAAACTGATTTCAGAAG
FQLSCY
C#QHAFR#GEHCLAF#qvqlqesggvlvqagdslrlscaasgFQLSCYCm
gwfrqapgkerefvasitQHAFRtyyadsvkgrftisrddarntvylqmn
slkpedtavyycaaGEHCLAFaywgqgtqvtvss#AGATATACCATGCAG
GTTCAACTGCAAGAATCTGGTGGCGTTTTAGTTCAAGCCGGTGATAGCCT
TCGTCTGAGCTGCGCCGCAAGCGGATTTCAGCTCTCGTGTTACTGTATGG
GTTGGTTTCGCCAGGCACCTGGTAAAGAACGTGAATTTGTGGCATCCATT
ACCCAGCACGCCTTTCGTACCTACTATGCCGATTCTGTTAAAGGTCGCTT
CACTATTTCACGCGATGATGCGCGTAATACCGTGTACTTACAGATGAACT
CACTGAAACCTGAGGATACCGCCGTTTATTATTGTGCGGCAGGCGAGCAC
TGTCTCGCCTTCGCCTATTGGGGTCAGGGCACACAAGTCACGGTCTCAAG
CGGCGGAAGCTCTGGTGAACAGAAACTGATTTCAGAAG
FQLSCY
C#QHAFR#AETPNILVYE#evqlvesggglvqagdslrlscaasgFQLSC
YCmgwfrqapgkerefvasitQHAFRtyyadsvkgrftisrddarntvyl
qmnslkpedtavyycaaAETPNILVYEnywgqgtqgtvss#ACCATGGAA
GTTCAACTGGTTGAATCTGGTGGCGGTTTAGTTCAAGCCGGTGATAGCCT
TCGTCTGAGCTGCGCCGCAAGCGGATTTCAGCTCTCGTGTTACTGTATGG
GTTGGTTTCGCCAGGCACCTGGTAAAGAACGTGAATTTGTGGCATCCATT
ACCCAGCACGCCTTTCGTACCTACTATGCCGATTCTGTTAAAGGTCGCTT
CACTATTTCACGCGATGATGCGCGTAATACCGTGTACTTACAGATGAACT
CACTGAAACCTGAGGATACCGCCGTTTATTATTGTGCGGCAGCCGAGACC
CCGAATATCCTCGTGTACGAGAACTATTGGGGTCAGGGCACACAAGGCAC
GGTCTCAAGCGGCGGAAGCTCTGGTGCACAGAAACTGATTTCAGAAGCCA
A
FQLSCYC#QHAFR#GEHCLAF#qvqlqesggvlvqagdslrls
caasgFQLSCYCmgwfrqapgkerefvasitQHAFRtyyadsvkgrftis
rddarntvylqmnslkpedtavyycaaGEHCLAFdywgqgtqgtvss#AT
ATACCATGCAGGTTCAACTGCAAGAATCTGGTGGCGTTTTAGTTCAAGCC
GGTGATAGCCTTCGTCTGAGCTGCGCCGCAAGCGGATTTCAGCTCTCGTG
TTACTGTATGGGTTGGTTTCGCCAGGCACCTGGTAAAGAACGTGAATTTG
TGGCATCCATTACCCAGCACGCCTTTCGTACCTACTATGCCGATTCTGTT
AAAGGTCGCTTCACTATTTCACGCGATGATGCGCGTAATACCGTGTACTT
ACAGATGAACTCACTGAAACCTGAGGATACCGCCGTTTATTATTGTGCGG
CAGGCGAGCACTGTCTCGCCTTCGACTATTGGGGTCAGGGCACACAAGGC
ACGGTCTCAAGCGGCGGAAGCTCTGGTGAACAGAAACTGATTTCAGAAGC
CAA
FQLSCYC#QHAFR#DPSFSDAIAS#evqlvesggglvqagd
slrlscaasgFQLSCYCmgwfrqapgkerefvasitQHAFRtyyadsvkg
rftisrddarntvylqmnslkpedtavyycavDPSFSDAIASdywgqgtq
vtfss#ACCATGGAAGTTCAACTGGTTGAATCTGGTGGCGGTTTAGTTCA
AGCCGGTGATAGCCTTCGTCTGAGCTGCGCCGCAAGCGGATTTCAGCTCT
CGTGTTACTGTATGGGTTGGTTTCGCCAGGCACCTGGTAAAGAACGTGAA
TTTGTGGCATCCATTACCCAGCACGCCTTTCGTACCTACTATGCCGATTC
TGTTAAAGGTCGCTTCACTATTTCACGCGATGATGCGCGTAATACCGTGT
ACTTACAGATGAACTCACTGAAACCTGAGGATACCGCCGTTTATTATTGT
GCGGTTGATCCTTCTTTCTCCGACGCTATCGCCTCGGACTATTGGGGTCA
GGGCACACAAGTCACGTTCTCAAGCGGCGGAAGCTCTGGTGAACAGAAAC
TGATTTCAGAAGCCAA
FQLSCYC#QHAFR#GEHCLAF#qvqlqe
sggvlvqagdslrlscaasgFQLSCYCmgwfrqapgkerefvasitQHAF
RtyyadsvkgrftisrddarntvylqmsslkpedtavyycaaGEHCLAFd
ywgqgtqvtvss#GGAGATATACCATGCAGGTTCAACTGCAAGAATCTGG
TGGCGTTTTAGTTCAAGCCGGTGATAGCCTTCGTCTGAGCTGCGCCGCAA
GCGGATTTCAGCTCTCGTGTTACTGTATGGGTTGGTTTCGCCAGGCACCT
GGTAAAGAACGTGAATTTGTGGCATCCATTACCCAGCACGCCTTTCGTAC
CTACTATGCCGATTCTGTTAAAGGTCGCTTCACTATTTCACGCGATGATG
CGCGTAATACCGTGTACTTACAGATGAGCTCACTGAAACCTGAGGATACC
GCCGTTTATTATTGTGCGGCAGGCGAGCACTGTCTCGCCTTCGACTATTG
GGGTCAGGGCACACAAGTCACGGTCTCAAGCGGCGGAAGCTCTGGTGAAC
AGAAACTGATTTCAGAAGCCAA
FQLSCYC#QHAFR#AETPNILV
YE#evqlvesggglvqagdslrlscaasgFQLSCYCmgwfrqapgkeref
vasitQHAFRtyyadsvkgrftisrddarntvylqmnslkpedtavyyca
aAETPNILVYEnywgqdtqvtvss#GAAGGAGATATACCATGGAAGTTCA
ACTGGTTGAATCTGGTGGCGGTTTAGTTCAAGCCGGTGATAGCCTTCGTC
TGAGCTGCGCCGCAAGCGGATTTCAGCTCTCGTGTTACTGTATGGGTTGG
TTTCGCCAGGCACCTGGTAAAGAACGTGAATTTGTGGCATCCATTACCCA
GCACGCCTTTCGTACCTACTATGCCGATTCTGTTAAAGGTCGCTTCACTA
TTTCACGCGATGATGCGCGTAATACCGTGTACTTACAGATGAACTCACTG
AAACCTGAGGATACCGCCGTTTATTATTGTGCGGCAGCCGAGACCCCGAA
TATCCTCGTGTACGAGAACTATTGGGGTCAGGACACACAAGTCACGGTCT
CAAGCGGCGGAAGCTCTGGTGAACAGAAACTGATTTCAGAAGCCAA
FQLSCYC#QHAFR#AETPNILVYE#evqlvesggglvqagdslrlsca
asgFQLSCYCmgwfrqapgkerefvasitQHAFRtyyadsvkgrftisrd
darntvylqmnslkpedtavyycaaAETPNILVYEnywgqgtrvtvss#A
GGAGATATACCATGGAAGTTCAACTGGTTGAATCTGGTGGCGGTTTAGTT
CAAGCCGGTGATAGCCTTCGTCTGAGCTGCGCCGCAAGCGGATTTCAGCT
CTCGTGTTACTGTATGGGTTGGTTTCGCCAGGCACCTGGTAAAGAACGTG
AATTTGTGGCATCCATTACCCAGCACGCCTTTCGTACCTACTATGCCGAT
TCTGTTAAAGGTCGCTTCACTATTTCACGCGATGATGCGCGTAATACCGT
GTACTTACAGATGAACTCACTGAAACCTGAGGATACCGCCGTTTATTATT
GTGCGGCAGCCGAGACCCCGAATATCCTCGTGTACGAGAACTATTGGGGT
CAGGGCACACGAGTCACGGTCTCAAGCGGCGGAAGCTCTGGTGAACAGAA
ACTGATTTCAGAAGCCAA
FQLSCYC#QHAFR#GEHCLAF#qvql
qescgvlvqvgdslrlscaasgFQLSCYCmgwfrqapgkerefvasitQH
AFRtyyadsvkgrftisrddarntvylqmnslkpedtavyycaaGEHCLA
Fdywgqgtqvtvss#GAAGGAGATATACCATGCAGGTTCAACTGCAAGAA
TCTTGTGGCGTTTTAGTTCAAGTCGGTGATAGCCTTCGTCTGAGCTGCGC
CGCAAGCGGATTTCAGCTCTCGTGTTACTGTATGGGTTGGTTTCGCCAGG
CACCTGGTAAAGAACGTGAATTTGTGGCATCCATTACCCAGCACGCCTTT
CGTACCTACTATGCCGATTCTGTTAAAGGTCGCTTCACTATTTCACGCGA
TGATGCGCGTAATACCGTGTACTTACAGATGAACTCACTGAAACCTGAGG
ATACCGCCGTTTATTATTGTGCGGCAGGCGAGCACTGTCTCGCCTTCGAC
TATTGGGGTCAGGGCACACAAGTCACGGTCTCAAGCGGCGGAAGCTCTGG
TGAACAGAAACTGATTTCAGAAGCCAA
FQLSCYC#QHSFR#DPS
FSDAIVS#evqlvesggglvqagdslrlscaasgFQLSCYCmgwfrqapg
kerefvasitQHSFRtyyadsvkgrftisrddarntvylqmnslkpedta
vyycavGCGGATTTCAGCTCTCGTGTTACTGTATGGGTTGGTTTCGCCAG
GCACCTGGTAAAGAACGTGAATTTGTGGCATCCATTACCCAGCACTCCTT
TCGTACCTACTATGCCGATTCTGTTAAAGGTCGCTTCACTATTTCACGCG
ATGATGCGCGTAATACCGTGTACTTACAGATGAACTCACTGAAACCTGAG
GATACCGCCGTTTATTATTGTGCGGTTGATCCTTCTTTCTCCGACGCTAT
CGTCTCGGACTATTGGGGTCAGGGCACACAAGTCACGGTCTCAAGCGGCG
GAAGCTCTGGTGAACAGAAACTGATTTCAGAAGCCAA
FQLSCYCQHAFR#DPSFSDAIAS#evqlvesggglvqagdslrlscaasg
FQLSCY
CmgwfrqapgkerefvasitQHAFRtyyadsvkgrftisrddarntvylh
mnslkpedtavyycavDPSFSDAIASdywgqgtqvtvss#GAAGGAGATA
TACCATGGAAGTTCAACTGGTTGAATCTGGTGGCGGTTTAGTTCAAGCCG
GTGATAGCCTTCGTCTGAGCTGCGCCGCAAGCGGATTTCAGCTCTCGTGT
TACTGTATGGGTTGGTTTCGCCAGGCACCTGGTAAAGAACGTGAATTTGT
GGCATCCATTACCCAGCACGCCTTTCGTACCTACTATGCCGATTCTGTTA
AAGGTCGCTTCACTATTTCACGCGATGATGCGCGTAATACCGTGTACTTA
CACATGAACTCACTGAAACCTGAGGATACCGCCGTTTATTATTGTGCGGT
TGATCCTTCTTTCTCCGACGCTATCGCCTCGGACTATTGGGGTCAGGGCA
CACAAGTCACGGTCTCAAGCGGCGGAAGCTCTGGTGAACAGAAACTGATT
TCAGAAGCCAA
FQLSCYC#QHAFR#GEHCLAF#qvqlqesggvl
vqagdglrlscaasgFQLSCYCmgwfrqapgkerefvasitQHAFRtyya
dsvkgrftisrddarntvylqmnslkpedtavyycaaGEHCLAFdywgqg
tqvtvss#AGGAGATATACCATGCAGGTTCAACTGCAAGAATCTGGTGGC
GTTTTAGTTCAAGCCGGTGATGGCCTTCGTCTGAGCTGCGCCGCAAGCGG
ATTTCAGCTCTCGTGTTACTGTATGGGTTGGTTTCGCCAGGCACCTGGTA
AAGAACGTGAATTTGTGGCATCCATTACCCAGCACGCCTTTCGTACCTAC
TATGCCGATTCTGTTAAAGGTCGCTTCACTATTTCACGCGATGATGCGCG
TAATACCGTGTACTTACAGATGAACTCACTGAAACCTGAGGATACCGCCG
TTTATTATTGTGCGGCAGGCGAGCACTGTCTCGCCTTCGACTATTGGGGT
CAGGGCACACAAGTCACGGTCTCAAGCGGCGGAAGCTCTGGTGAACAGAA
ACTGATTTCAGAAGCCAA
FQLSCYC#QHAFR#GEHCLAF#qvql
qesggvlvqagdslrlscaasgFQLSCYCmgwfrqapgkerefvasitQH
AFRtyyadsvkgrftisrddarntvylqmnslkpedttvyycaaGEHCLA
Fdywgqgtqvtvss#AGGAGATATACCATGCAGGTTCAACTGCAAGAATC
TGGTGGCGTTTTAGTTCAAGCCGGTGATAGCCTTCGTCTGAGCTGCGCCG
CAAGCGGATTTCAGCTCTCGTGTTACTGTATGGGTTGGTTTCGCCAGGCA
CCTGGTAAAGAACGTGAATTTGTGGCATCCATTACCCAGCACGCCTTTCG
TACCTACTATGCCGATTCTGTTAAAGGTCGCTTCACTATTTCACGCGATG
ATGCGCGTAATACCGTGTACTTACAGATGAACTCACTGAAACCTGAGGAT
ACCACCGTTTATTATTGTGCGGCAGGCGAGCACTGTCTCGCCTTCGACTA
TTGGGGTCAGGGCACACAAGTCACGGTCTCAAGCGGCGGAAGCTCTGGTG
AACAGAAACTGATTTCAGAAGCCAA
FQLSCYC#QHAFR#GEHCL
AF#qvqlqesggvlvqagdslrlscaasgFQLSCYCmgwfrqapgkeref
vasitQHAFRtyyadsvkgrftisrddarntvylqmnslkpedtavyyca
aGEHCLAFdywgqgtqvtvts#AGGAGATATACCATGCAGGTTCAACTGC
AAGAATCTGGTGGCGTTTTAGTTCAAGCCGGTGATAGCCTTCGTCTGAGC
TGCGCCGCAAGCGGATTTCAGCTCTCGTGTTACTGTATGGGTTGGTTTCG
CCAGGCACCTGGTAAAGAACGTGAATTTGTGGCATCCATTACCCAGCACG
CCTTTCGTACCTACTATGCCGATTCTGTTAAAGGTCGCTTCACTATTTCA
CGCGATGATGCGCGTAATACCGTGTACTTACAGATGAACTCACTGAAACC
TGAGGATACCGCCGTTTATTATTGTGCGGCAGGCGAGCACTGTCTCGCCT
TCGACTATTGGGGTCAGGGCACACAAGTCACGGTCACAAGCGGCGGAAGC
TCTGGTGAACAGAAACTGATTTCAGAAGCCAA
FQLSCYC#QHAF
R#DPSFSDAIAS#evqlvesggglvqagdslrlscaasgFQLSCYCmgwf
rqapgkerefvasitQHAFRtyyadsvkgrftisrddarntvylqmnslk
pedtavyycavDPSFSDAIASdywgqgtqgtvss#AGATATACCATGGAA
GTTCAACTGGTTGAATCTGGTGGCGGTTTAGTTCAAGCCGGTGATAGCCT
TCGTCTGAGCTGCGCCGCAAGCGGATTTCAGCTCTCGTGTTACTGTATGG
GTTGGTTTCGCCAGGCACCTGGTAAAGAACGTGAATTTGTGGCATCCATT
ACCCAGCACGCCTTTCGTACCTACTATGCCGATTCTGTTAAAGGTCGCTT
CACTATTTCACGCGATGATGCGCGTAATACCGTGTACTTACAGATGAACT
CACTGAAACCTGAGGATACCGCCGTTTATTATTGTGCGGTTGATCCTTCT
TTCTCCGACGCTATCGCCTCGGACTATTGGGGTCAGGGCACACAAGGCAC
GGTCTCAAGCGGCGGAAGCTCTGGTGAAAAGAAACTGATTTCAGAAGCCA
A
FQLSCYC#QHAFR#AETPNILVYE#evrlvesggglvqagdsl
rlscaasgFQLSCYCmgwfrqapgkerefvasitQHAFRtyyadsvkgrf
tisrddarntvylqmnslkpedtavyycaaAETPNILVYEnywgqgtqvt
vss#AGGAGATATACCATGGAAGTTCGACTGGTTGAATCTGGTGGCGGTT
TAGTTCAAGCCGGTGATAGCCTTCGTCTGAGCTGCGCCGCAAGCGGATTT
CAGCTCTCGTGTTACTGTATGGGTTGGTTTCGCCAGGCACCTGGTAAAGA
ACGTGAATTTGTGGCATCCATTACCCAGCACGCCTTTCGTACCTACTATG
CCGATTCTGTTAAAGGTCGCTTCACTATTTCACGCGATGATGCGCGTAAT
ACCGTGTACTTACAGATGAACTCACTGAAACCTGAGGATACCGCCGTTTA
TTATTGTGCGGCAGCCGAGACCCCGAATATCCTCGTGTACGAGAACTATT
GGGGTCAGGGCACACAAGTCACGGTCTCAAGCGGCGGAAGCTCTGGTGAA
CAGAAACTGATTTCAGAAGCCAA
FPLSCYC#QHAFR#AETPNIL
VYE#evqlvesggglvqagdslrlscaasgFPLSCYCmgwfrqapgkere
fvasitQHAFRtyyadsvkgrftisrddarntvylqmnslkpedtavyyc
aaAETPNILVYEnywgqgtqvtvss#ATACCATGGAAGTTCAACTGGTTG
AATCTGGTGGCGGTTTAGTTCAAGCCGGTGATAGCCTTCGTCTGAGCTGC
GCCGCAAGCGGATTTCCGCTCTCGTGTTACTGTATGGGTTGGTTTCGCCA
GGCACCTGGTAAAGAACGTGAATTTGTGGCATCCATTACCCAGCACGCCT
TTCGTACCTACTATGCCGATTCTGTTAAAGGTCGCTTCACTATTTCACGC
GATGATGCGCGTAATACCGTGTACTTACAGATGAACTCACTGAAACCTGA
GGATACCGCCGTTTATTATTGTGCGGCAGCCGAGACCCCGAATATCCTCG
TGTACGAGAACTATTGGGGTCAGGGCACACAAGTCACGGTCTCAAGCGGC
GGAAGCTCTGGTGAACAGAAACTGATTTCAGAAGCCAA
FQLSCY
C#QHAFR#AETPNILVHE#evqlvesggglvqagdslrIncaasgFQLSC
YCmgwfrqapgkerefvasitQHAFRtyyadsvkgrftisrddarntvyl
qmnslkpedtavyycaaAETPNILVHEnywgqgtqgtvss#GGAGATATA
CCATGGAAGTTCAACTGGTTGAATCTGGTGGCGGTTTAGTTCAAGCCGGT
GATAGCCTTCGTCTGAACTGCGCCGCAAGCGGATTTCAGCTCTCGTGTTA
CTGTATGGGTTGGTTTCGCCAGGCACCTGGTAAAGAACGTGAATTTGTGG
CATCCATTACCCAGCACGCCTTTCGTACCTACTATGCCGATTCTGTTAAA
GGTCGCTTCACTATTTCACGCGATGATGCGCGTAATACCGTGTACCTACA
GATGAACTCACTGAAACCTGAGGATACCGCCGTTTATTATTGTGCGGCAG
CCGAGACCCCGAATATCCTCGTGCACGAGAACTATTGGGGTCAGGGCACA
CAAGGCACGGTCTCAAGCGGCGGAAGCTCTGGTGAACAGAAACTGATTTC
AGAAGCCAA
FQLSCYC#QHAFR#DPSFSDAIAS#evqlvesggg
lvkagdslrlscaasgFQLSCYCmgwfrqapgkerefvasitQHAFRtyy
adsvkgrftisrddarntvylqmnslkpedtavyycavDPSFSDAIASdy
wgqgtqvtvss#ACCATGGAAGTTCAACTGGTTGAATCTGGTGGCGGTTT
AGTTAAAGCCGGTGATAGCCTTCGTCTGAGCTGCGCCGCAAGCGGATTTC
AGCTCTCGTGTTACTGTATGGGTTGGTTTCGCCAGGCACCTGGTAAAGAA
CGTGAATTTGTGGCATCCATTACCCAGCACGCCTTTCGTACCTACTATGC
CGATTCTGTTAAAGGTCGCTTCACTATTTCACGCGATGATGCGCGTAATA
CCGTGTACTTACAGATGAACTCACTGAAACCTGAGGATACCGCCGTTTAT
TATTGTGCGGTTGATCCTTCTTTCTCCGACGCTATCGCCTCGGACTATTG
GGGTCAGGGCACACAAGTCACGGTCTCAAGCGGCGGAAGCTCTGGTGAAC
AGAAACTGATTTCAGAAGCCAA
FQLSCYC#QHAFR#AETPNILV
YE#evqlvdsggglvqagdrlrlscaasgFQLSCYCmgwfrqapgkeref
vasitQHAFRtyyadsvkgrftisrddarntvylqmnslkpedtavyyca
aAETPNILVYEnywgqgtqgtvss#GAAGGAGATATACCATGGAAGTTCA
ACTGGTTGACTCTGGTGGCGGTTTAGTTCAAGCCGGTGATCGCCTTCGTC
TGAGCTGCGCCGCAAGCGGATTTCAGCTCTCGTGTTACTGTATGGGTTGG
TTTCGCCAGGCACCTGGTAAAGAACGTGAATTTGTGGCATCCATTACCCA
GCACGCCTTTCGTACCTACTATGCCGATTCTGTTAAAGGTCGCTTCACTA
TTTCACGCGATGATGCGCGTAATACCGTGTACTTACAGATGAACTCACTG
AAACCTGAGGATACCGCCGTTTATTATTGTGCGGCAGCCGAGACCCCGAA
TATCCTCGTGTACGAGAACTATTGGGGTCAGGGCACACAAGGCACGGTCT
CAAGCGGCGGAAGCTCTGGTGAACAGAAACTGATTTCAGAAGCCAA
FQLSCSC#QHAFR#DPSFSDAIAS#evqlvesggglvqagdrlrlsca
asgFQLSCSCmgwfrqapgkerefvasitQHAFRtyyadsvkgrftisrd
darntvylqmnslkpedtavyycavDPSFSDAIASdywgqgtqgtvss#A
AGGAGATATACCATGGAAGTTCAACTGGTTGAATCTGGTGGCGGTTTAGT
TCAAGCCGGTGATAGACTTCGTCTGAGCTGCGCCGCAAGCGGATTTCAGC
TCTCGTGTTCCTGTATGGGTTGGTTTCGCCAGGCACCTGGTAAAGAACGT
GAATTTGTGGCATCCATTACCCAGCACGCCTTTCGTACCTACTATGCCGA
TTCTGTTAAAGGTCGCTTCACTATTTCACGCGATGATGCGCGTAATACCG
TGTACTTACAGATGAACTCACTGAAACCTGAGGATACCGCCGTTTATTAT
TGTGCGGTTGATCCTTCTTTCTCCGACGCTATCGCCTCGGACTATTGGGG
TCAGGGCACACAAGGCACGGTCTCAAGCGGCGGAAGCTCTGGTGAACAGA
AACTGATTTCAGAAGCCAA
FQLSCYC#QHAFR#AETPNILVYE#
evqlvesggglvqagdslrlscaasgFQLSCYCmgwfrqapgkerefvat
itQHAFRtyyadsvkgrftisrddarntvylqmnslkpedtavyycaaAE
TPNILVYEnywgqgtqvtvss#ATACCATGGAAGTTCAACTGGTTGAATC
TGGTGGCGGTTTAGTTCAAGCCGGTGATAGCCTTCGTCTGAGCTGCGCCG
CAAGCGGATTTCAGCTCTCGTGTTACTGTATGGGTTGGTTTCGCCAGGCA
CCTGGTAAAGAACGTGAATTTGTGGCAACCATTACCCAGCACGCCTTTCG
TACCTACTATGCCGATTCTGTTAAAGGTCGCTTCACTATTTCACGCGATG
ATGCGCGTAATACCGTGTACTTACAGATGAACTCACTGAAACCTGAGGAT
ACCGCCGTTTATTATTGTGCGGCAGCCGAGACCCCGAATATCCTCGTGTA
CGAGAACTATTGGGGTCAGGGCACACAAGTCACGGTCTCAAGCGGCGGAA
GCTCTGGTGAACAGAAACTGATTTCAGAAGCCAA
FQLSCYC#QH
AFR#DPSFSDAIAS#evqlvesggglvhagdrlrlscaasgFQLSCYCmg
wfrqapgkerefvasitQHAFRtyyadsvkgrftisrddarntvylqmns
lkpedtavyycavDPSFSDAIASdywgqgtqvtvss#GAAGGAGATATAC
CATGGAAGTTCAACTGGTTGAATCTGGTGGCGGTTTAGTTCACGCCGGTG
ATCGCCTTCGTCTGAGCTGCGCCGCAAGCGGATTTCAGCTCTCGTGTTAC
TGTATGGGTTGGTTTCGCCAGGCACCTGGTAAAGAACGTGAATTTGTGGC
ATCCATTACCCAGCACGCCTTTCGTACCTACTATGCCGATTCTGTTAAAG
GTCGCTTCACTATTTCACGCGATGATGCGCGTAATACCGTGTACTTACAG
ATGAACTCACTGAAACCTGAGGATACCGCCGTTTATTATTGTGCGGTTGA
TCCTTCTTTCTCCGACGCTATCGCCTCGGACTATTGGGGTCAGGGCACAC
AAGTCACGGTCTCAAGCGGCGGAAGCTCTGGTGAACAGAAACTGATTTCA
GAAGCCAA
FQLSCYC#QHAFR#AETPNILVYE#evqlvesgggl
vqagdslrlscaasgFQLSCYCmgwfrqapgkerefvasitQHAFRtyya
dsvkgrftisrddarntvylqmnspkpedtavyycaaAETPNILVYEnyw
gqgtqvtvss#ACCATGGAAGTTCAACTGGTTGAATCTGGTGGCGGTTTA
GTTCAAGCCGGTGATAGCCTTCGTCTGAGCTGCGCCGCAAGCGGATTTCA
GCTCTCGTGTTACTGTATGGGTTGGTTTCGCCAGGCACCTGGTAAAGAAC
GTGAATTTGTGGCATCCATTACCCAGCACGCCTTTCGTACCTACTATGCC
GATTCTGTTAAAGGTCGCTTCACTATTTCACGCGATGATGCGCGTAATAC
CGTGTACTTACAGATGAACTCACCGAAACCTGAGGATACCGCCGTTTATT
ATTGTGCGGCAGCCGAGACCCCGAATATCCTCGTGTACGAGAACTATTGG
GGTCAGGGCACACAAGTCACGGTCTCAAGCGGCGGAAGCTCTGGTGAACA
GAAACTGATTTCAGAAGCCAA
FQLSCYC#QHAFR#GEHCLAF#q
vqlqesggvlvhagdrlrlscaasgFQLSCYCmgwfrqapgkerefvasi
tQHAFRtyyadsvkgrftisrddarntvylqmnslkpedtavyycaaGEH
CLAFdywgqgtqvtvss#GAAGGAGATATACCATGCAGGTTCAACTGCAA
GAATCTGGTGGCGTTTTAGTTCACGCCGGTGATCGCCTTCGTCTGAGCTG
CGCCGCAAGCGGATTTCAGCTCTCGTGTTACTGTATGGGTTGGTTTCGCC
AGGCACCTGGTAAAGAACGTGAATTTGTGGCATCCATTACCCAGCACGCC
TTTCGTACCTACTATGCCGATTCTGTTAAAGGTCGCTTCACTATTTCACG
CGATGATGCGCGTAATACCGTGTACTTACAGATGAACTCACTGAAACCTG
AGGATACCGCCGTTTATTATTGTGCGGCAGGCGAGCACTGTCTCGCCTTC
GACTATTGGGGTCAGGGCACACAAGTCACGGTCTCAAGCGGCGGAAGCTC
TGGTGAACAGAAACTGATTTCAGAAGCCAA

TABLE 4
Sequences belonging to cluster SR6. Each line
in the table represents one sequence, both
segments and full length of the sequence are
shown. Shown items are divided by “#” and in
the order from start to end of each line is:
CDR1 amino acid sequence, CDR2 amino acid
sequence, CDR3 amino acid sequence, full-length
amino acid sequence with CDRs capitalized,
full-length DNA sequence.
(SEQ ID NOS: 691-1420)

DCHVLWR#LDGLF#MPFF#evqlvesggglvqagdslrlscaasgDCHVL
WRmgwfrqapgkerefvaaisLDGLFtnyadsvrgrfsisadsakntvyl
qmnslkpedtavyycaaMPFFdywgqgtqvtvss#ACCATGGAAGTTCAA
CTGGTTGAATCTGGTGGCGGTTTAGTTCAAGCCGGTGATAGCCTTCGTCT
GAGCTGCGCCGCAAGCGGAGATTGTCACGTTCTCTGGCGTATGGGTTGGT
TTCGCCAGGCACCTGGTAAAGAACGTGAATTTGTGGCAGCCATTAGCCTC
GACGGTTTGTTTACCAACTATGCCGATTCTGTTCGTGGTCGCTTCAGCAT
TTCAGCAGATTCTGCGAAGAATACCGTGTACTTACAGATGAACTCACTGA
AACCTGAGGATACCGCCGTTTATTATTGTGCGGCAATGCCTTTTTTTGAC
TATTGGGGTCAGGGCACACAAGTCACGGTCTCAAGCGGCGGAAGCTCTGG
TGAACAGAAACTGATTTCAGAAG
DCHVLWR#LDGLF#LPATSG#
evqlvesggglvqagdslrlscaasgDCHVLWRmgwfrqapgkerefvaa
isLDGLFtnyadsvrgrfsisadsakntvylqmnslkpedtavyycaaLP
ATSGdywgqgtqvtvss#AAGGAGATATACCATGGAAGTTCAACTGGTTG
AATCTGGTGGCGGTTTAGTTCAAGCCGGTGATAGCCTTCGTCTGAGCTGC
GCCGCAAGCGGAGATTGTCACGTTCTCTGGCGTATGGGTTGGTTTCGCCA
GGCACCTGGTAAAGAACGTGAATTTGTGGCAGCCATTAGCCTCGACGGTT
TGTTTACCAACTATGCCGATTCTGTTCGTGGTCGCTTCAGCATTTCAGCA
GATTCTGCGAAGAATACCGTGTACTTACAGATGAACTCACTGAAACCTGA
GGATACCGCCGTTTATTATTGTGCGGCATTGCCGGCGACTTCTGGCGACT
ATTGGGGTCAGGGCACACAAGTCACGGTCTCAAGCGGCGGAAGCTCTGGT
GAACAGAAACTGATTTCAGAAG
DCHVLWR#LDGLF#FKPYES#e
vqlvesggglvqagdslrlscaasgDCHVLWRmgwfrqapgkerefvaai
sLDGLFtnyadsvrgrfsisadsakntvylqmnslkpedtavyycaaFKP
YESaywgqgtqvtvss#ATACCATGGAAGTTCAACTGGTTGAATCTGGTG
GCGGTTTAGTTCAAGCCGGTGATAGCCTTCGTCTGAGCTGCGCCGCAAGC
GGAGATTGTCACGTTCTCTGGCGTATGGGTTGGTTTCGCCAGGCACCTGG
TAAAGAACGTGAATTTGTGGCAGCCATTAGCCTCGACGGTTTGTTTACCA
ACTATGCCGATTCTGTTCGTGGTCGCTTCAGCATTTCAGCAGATTCTGCG
AAGAATACCGTGTACTTACAGATGAACTCACTGAAACCTGAGGATACCGC
CGTTTATTATTGTGCGGCATTCAAGCCTTACGAGTCGGCCTATTGGGGTC
AGGGCACACAAGTCACGGTCTCAAGCGGCGGAAGCTCTGGTGAACAGAAA
CTGATTTCAGAAG
DCHVLWR#LVGLF#MPFF#evqlvesggglv
qagdslrlscaasgDCHVLWRmgwfrqapgkerefvaaisLVGLFtnyad
svrgrfsisadsakntvylqmnslkpedtavyycaaMPFFdywgqgtqvt
vss#ACCATGGAAGTTCAACTGGTTGAATCTGGTGGCGGTTTAGTTCAAG
CCGGTGATAGCCTTCGTCTGAGCTGCGCCGCAAGCGGAGATTGTCACGTT
CTCTGGCGTATGGGTTGGTTTCGCCAGGCACCTGGTAAAGAACGTGAATT
TGTGGCAGCCATTAGCCTCGTCGGTTTGTTTACCAACTATGCCGATTCTG
TTCGTGGTCGCTTCAGCATTTCAGCAGATTCTGCGAAGAATACCGTGTAC
TTACAGATGAACTCACTGAAACCTGAGGATACCGCCGTTTATTATTGTGC
GGCAATGCCTTTTTTTGACTATTGGGGTCAGGGCACACAAGTCACGGTCT
CAAGCGGCGGAAGCTCTGGTGAACAGAAACTGATTTCAGAAG
DC
HVLWR#LDGLF#HHFHPGGMD#evqlvesggglvqagdslrlscaasgDC
HVLWRmgwfrqapgkerefvaaisLDGLFtnyadsvrgrfsisadsaknt
vylqmnslkpedtavyycnaHHFHPGGMDdywgqgtqvtvss#ATACCAT
GGAAGTTCAACTGGTTGAATCTGGTGGCGGTTTAGTTCAAGCCGGTGATA
GCCTTCGTCTGAGCTGCGCCGCAAGCGGAGATTGTCACGTTCTCTGGCGT
ATGGGTTGGTTTCGCCAGGCACCTGGTAAAGAACGTGAATTTGTGGCAGC
CATTAGCCTCGACGGTTTGTTTACCAACTATGCCGATTCTGTTCGTGGTC
GCTTCAGCATTTCAGCAGATTCTGCGAAGAATACCGTGTACTTACAGATG
AACTCACTGAAACCTGAGGATACCGAACAGAAACTGATTTCAGAAG
DCHVLWR#LDGLF#LPKRTV#qvqlvesggalvqagdslrlscaasgD
CHVLWRmgwfrqapgkerefvaaisLDGLFtnyadsvrgrfsisadsakn
tvylqmnslkpedtavyycaaLPKRTVdywgqgtqvtvss#AAGGAGATA
TACCATGCAGGTTCAACTGGTTGAATCTGGTGGCGCATTAGTTCAAGCCG
GTGATAGCCTTCGTCTGAGCTGCGCCGCAAGCGGAGATTGTCACGTTCTC
TGGCGTATGGGTTGGTTTCGCCAGGCACCTGGTAAAGAACGTGAATTTGT
GGCAGCCATTAGCCTCGACGGTTTGTTTACCAACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGGC
ACTGCCTAAGCGGACTGTGGACTATTGGGGTCAGGGCACACAAGTCACGG
TCTCAAGCGGCGGAAGCTCTGGTGAACAGAAACTGATTTCAGAAG
DCHVLWR#LDGLF#LPATSG#evqlvesggglvhvgdrlrlscaasgDC
HVLWRmgwfrqapgkerefvaaisLDGLFtnyadsvrgrfsisadsaknt
vylqmnslkpedtavyycaaLPATSGdywgqgtqvtvss#GAAGGAGATA
TACCATGGAAGTTCAACTGGTTGAATCTGGTGGCGGTTTAGTTCACGTCG
GTGATCGCCTTCGTCTGAGCTGCGCCGCAAGCGGAGATTGTCACGTTCTC
TGGCGTATGGGTTGGTTTCGCCAGGCACCTGGTAAAGAACGTGAATTTGT
GGCAGCCATTAGCCTCGACGGTTTGTTTACCAACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGGC
ATTGCCGGCGACTTCTGGCGACTATTGGGGTCAGGGCACACAAGTCACGG
TCTCAAGCGGCGGAAGCTCTGGTGAACAGAAACTGATTTCAGAAG
DCHVLWR#LDGLF#MPFF#evqlvesggglvqagdslrlscaasgDCHV
LWRmgwfrqapgkerefvaaisLDGLFtnyadsvrgrfsisadsakntvy
lqmnslkpedtavyycaaMPFFaywgqgtqvtvss#AAGGAGATATACCA
TGGAAGTTCAACTGGTTGAATCTGGTGGCGGTTTAGTTCAAGCCGGTGAT
AGCCTTCGTCTGAGCTGCGCCGCAAGCGGAGATTGTCACGTTCTCTGGCG
TATGGGTTGGTTTCGCCAGGCACCTGGTAAAGAACGTGAATTTGTGGCAG
CCATTAGCCTCGACGGTTTGTTTACCAACTATGCCGATTCTGTTCGTGGT
CGCTTCAGCATTTCAGCAGATTCTGCGAAGAATACCGTGTACTTACAGAT
GAACTCACTGAAACCTGAGGATACCGCCGTTTATTATTGTGCGGCAATGC
CTTTTTTTGCCTATTGGGGTCAGGGCACACAAGTCACGGTCTCAAGCGGC
GGAAGCTCTGGTGAACAGAAACTGATTTCAGAAG
DCHVLWR#LD
GLF#MPFF#evqlvesggglvqagdslrlscaasgDCHVLWRmgwfhqap
gkerefvaaisLDGLFtnyadsvrgrfsisadsakntvylqmnslkpedt
avyycaaMPFFdywgqgtqvtvss#ATACCATGGAAGTTCAACTGGTTGA
ATCTGGTGGCGGTTTAGTTCAAGCCGGTGATAGCCTTCGTCTGAGCTGCG
CCGCAAGCGGAGATTGTCACGTTCTCTGGCGTATGGGTTGGTTTCACCAG
GCACCTGGTAAAGAACGTGAATTTGTGGCAGCCATTAGCCTCGACGGTTT
GTTTACCAACTATGCCGATTCTGTTCGTGGTCGCTTCAGCATTTCAGCAG
ATTCTGCGAAGAATACCGTGTACTTACAGATGAACTCACTGAAACCTGAG
GATACCGCCGTTTATTATTGTGCGGCAATGCCTTTTTTTGACTATTGGGG
TCAGGGCACACAAGTCACGGTCTCAAGCGGCGGAAGCTCTGGTGAACAGA
AACTGATTTCAGAAG
DCHVLWR#LDGLF#HHFHPGGMD#evqlv
esggglvqagdslrlscaasgDCHVLWRmgwfrqapgkerefvaaisLDG
LFtnyadsvrgrfsisadsakntvylqmnslkpedtavyycnaHHFHPGG
MDaywgqgtqvtvss#AGGAGATATACCATGGAAGTTCAACTGGTTGAAT
CTGGTGGCGGTTTAGTTCAAGCCGGTGATAGCCTTCGTCTGAGCTGCGCC
GCAAGCGGAGATTGTCACGTTCTCTGGCGCATGGGTTGGTTTCGCCAGGC
ACCTGGTAAAGAACGTGAATTTGTGGCAGCCATTAGCCTCGACGGTTTGT
TTACCAACTATGCCGATTCTGTTCGTGGTCGCTTCAGCATTTCAGCAGAT
TCTGCGAAGAATACCGTGTACTTACAGATGAACTCACTGAAACCTGAGGA
TACCGCCGTTTATTATTGTAATGCACACCATTTTCATCCTGGTGGTATGG
ATGCCTATTGGGGTCAGGGCACACAAGTCACGGTCTCAAGCGGCGGAAGC
TCTGGTGAACAGAAACTGATTTCAGAAG
DCHVLWR#LDGLF#MP
FF#evqlvesggglvqagdslrlscaasgDCHVLWRmdwfrqapgkeref
vaaisLDGLFtnyadsvrgrfsisadsakntvylqmnslkpedtavyyca
aMPFFdywgqgtqvtvss#GGAGATATACCATGGAAGTTCAACTGGTTGA
ATCTGGTGGCGGTTTAGTTCAAGCCGGTGATAGCCTTCGTCTGAGCTGCG
CCGCAAGCGGAGATTGTCACGTTCTCTGGCGTATGGATTGGTTTCGCCAG
GCACCTGGTAAAGAACGTGAATTTGTGGCAGCCATTAGCCTCGACGGTTT
GTTTACCAACTATGCCGATTCTGTTCGTGGTCGCTTCAGCATTTCAGCAG
ATTCTGCGAAGAATACCGTGTACTTACAGATGAACTCACTGAAACCTGAG
GATACCGCCGTTTATTATTGTGCGGCAATGCCTTTTTTTGACTATTGGGG
TCAGGGCACACAAGTCACGGTCTCGAGCGGCGGAAGCTCTGGTGAACAGA
AACTGATTTCAGAAG
DCHVLWR#LDGLF#MPFF#evqlvesggv
lvqagdslrlscaasgDCHVLWRmgwfrqapgkerefvaaisLDGLFtny
adsvrgrfsisadsakntvylqmnslkpedtavyycaaMPFFdywgqgtq
vtvss#GAAGGAGATATACCATGGAAGTTCAACTGGTTGAATCTGGTGGC
GTTTTAGTTCAAGCCGGTGATAGCCTTCGTCTGAGCTGCGCCGCAAGCGG
AGATTGTCACGTTCTCTGGCGTATGGGTTGGTTTCGCCAGGCACCTGGTA
AAGAACGTGAATTTGTGGCAGCCATTAGCCTCGACGGTTTGTTTACCAAC
TATGCCGATTCTGTTCGTGGTCGCTTCAGCATTTCAGCAGATTCTGCGAA
GAATACCGTGTACTTACAGATGAACTCACTGAAACCTGAGGATACCGCCG
TTTATTATTGTGCGGCAATGCCTTTTTTTGACTATTGGGGTCAGGGCACA
CAAGTCACGGTCTCAAGCGGCGGAAGCTCTGGTGAACAGAAACTGATTTC
AGAAG
DCHVLWR#LDGLF#LPSVCT#evqlvesggglvqagdsl
rlscaasgDCHVLWRmgwfrqapgkerefvaaisLDGLFtnyadsvrgrf
sisadsakntvylqmnslkpedtavyycaaLPSVCTdywgqgtqvtvss#
ATACCATGGAAGTTCAACTGGTTGAATCTGGTGGCGGTTTAGTTCAAGCC
GGTGATAGCCTTCGTCTGAGCTGCGCCGCAAGCGGAGATTGTCACGTTCT
CTGGCGTATGGGTTGGTTTCGCCAGGCACCTGGTAAAGAACGTGAATTTG
TGGCAGCCATTAGCCTCGACGGTTTGTTTACCAACTATGCCGATTCTGTT
CGTGGTCGCTTCAGCATTTCAGCAGATTCTGCGAAGAATACCGTGTACTT
ACAGATGAACTCACTGAAACCTGAGGATACCGCCGTTTATTATTGTGCGG
CACTGCCTAGTGTGTGTACCGACTATTGGGGTCAGGGCACACAAGTCACG
GTCTCAAGCGGCGGAAGCTCTGGTGAACAGAAACTGATTTCAGAAG
DCHVLWR#LDGLF#LPATSG#evqlvesggglvqagdslrlscaasgD
CHVLWRmgwfsqapgkerefvaaisLDGLFtnyadsvrgrfsisadsakn
tvdvqmnslkpedtavyycaaLPATSGdywgqgtqvtvss#AAGGAGATA
TACCATGGAAGTTCAACTGGTTGAATCTGGTGGCGGTTTAGTTCAAGCCG
GTGATAGCCTTCGTCTGAGCTGCGCCGCAAGCGGAGATTGTCACGTTCTC
TGGCGTATGGGTTGGTTTAGCCAGGCACCTGGTAAAGAACGTGAATTTGT
GGCAGCCATTAGCCTCGACGGTTTGTTTACCAACTATGCCGATTCTGTTC
GTGGTCGCTTCAGCATTTCAGCAGATTCTGCGAAGAATACCGTGGACGTA
CAGATGAACTCACTGAAACCTGAGGATACCGCCGTTTATTATTGTGCGGC
ATTGCCGGCGACTTCTGGCGACTATTGGGGTCAGGGCACACAAGTCACGG
TCTCAAGCGGCGGAAGCTCTGGTGAACAGAAACTAATTTCAGAAG
DCHVLWR#LDGLF#MPFF#evqlvesggglvqagdslrlscaasgDCHV
LWRmgwfrqapgkerefvaaisLDGLFtnyadsvrgrfsisadsakntmy
lqmnslkpedtavyycaaMPFFdywgqgtqvtvss#ATATACCATGGAAG
TTCAACTGGTTGAATCTGGTGGCGGTTTAGTTCAAGCCGGTGATAGCCTT
CGTCTGAGCTGCGCCGCAAGCGGAGATTGTCACGTTCTCTGGCGTATGGG
TTGGTTTCGCCAGGCACCTGGTAAAGAACGTGAATTTGTGGCAGCCATTA
GCCTCGACGGTTTGTTTACCAACTATGCCGATTCTGTTCGTGGTCGCTTC
AGCATTTCAGCAGATTCTGCGAAGAATACCATGTACTTACAGATGAACTC
ACTGAAACCTGAGGATACCGCCGTTTATTATTGTGCGGCAATGCCTTTTT
TTGACTATTGGGGTCAGGGCACACAAGTCACGGTCTCAAGCGGCGGAAGC
TCTGGTGAACAGAAACTGATTTCAGAAG
DCHVLWR#LDGLF#LP
ATSG#evqlvesggglvqagdslrlscaasgDCHVLWRmgwfrqapgker
efvaaisLDGLFtnyadsvrgrfsisadsakntvylqmnslkpedtavyy
catLPATSGdywgqgtqvtvss#ACCATGGAAGTTCAACTGGTTGAATCT
GGTGGCGGTTTAGTTCAAGCCGGTGATAGCCTTCGTCTGAGCTGCGCCGC
AAGCGGAGATTGTCACGTTCTCTGGCGTATGGGTTGGTTTCGCCAGGCAC
CTGGTAAAGAACGTGAATTTGTGGCAGCCATTAGCCTCGACGGTTTGTTT
ACCAACTATGCCGATTCTGTTCGTGGTCGCTTCAGCATTTCAGCAGATTC
TGCGAAGAATACCGTGTACTTACAGATGAACTCACTGAAACCTGAGGATA
CCGCCGTTTATTATTGTGCGACATTGCCGGCGACTTCTGGCGACTATTGG
GGTCAGGGCACACAAGTCACGGTCTCAAGCGGCGGAAGCTCTGGTGAACA
GAAACTGATTTCAGAAG
DCHVLWR#LDGLF#MPFF#evqlfesg
gglvqagdslrlscaasgDCHVLWRmgwfrqapgkerefvaaisLDGLFt
nyadsvrgrfsisadsakntvylqmnslkpedtavyycaaMPFFdywgqg
tqvtvss#ATATACCATGGAAGTTCAACTGTTTGAATCTGGTGGCGGTTT
AGTTCAAGCCGGTGATAGCCTTCGTCTGAGCTGCGCCGCAAGCGGAGATT
GTCACGTTCTCTGGCGTATGGGTTGGTTTCGCCAGGCACCTGGTAAAGAA
CGTGAATTTGTGGCAGCCATTAGCCTCGACGGTTTGTTTACCAACTATGC
CGATTCTGTTCGTGGTCGCTTCAGCATTTCAGCAGATTCTGCGAAGAATA
CCGTGTACTTACAGATGAACTCACTGAAACCTGAGGATACCGCCGTTTAT
TATTGTGCGGCAATGCCTTTTTTTGACTATTGGGGTCAGGGCACACAAGT
CACGGTCTCAAGCGGCGGAAGCTCTGGTGAACAGAAACTGATTTCAGAAG
DCHVLWR#LDGLF#LPSVCT#evqlvesggglvqagdslrlsca
asgDCHVLWRmgcfrqapgkerefvaaisLDGLFtnyadsvrgrfsisad
sakntvylqmnslkpedtavyycaaLPSVCTdywgqgtqvtvss#AAGGA
GATATACCATGGAAGTTCAACTGGTTGAATCTGGTGGCGGTTTAGTTCAA
GCCGGTGATAGCCTTCGTCTGAGCTGCGCCGCAAGCGGAGATTGTCACGT
TCTCTGGCGTATGGGTTGTTTTCGCCAGGCACCTGGTAAAGAACGTGAAT
TTGTGGCAGCCATTAGCCTCGACGGTTTGTTTACCAACTATGCCGATTCT
GTTCGTGGTCGCTTCAGCATTTCAGCAGATTCTGCGAAGAATACCGTGTA
CTTACAGATGAACTCACTGAAACCTGAGGATACCGCCGTTTATTATTGTG
CGGCACTGCCTAGTGTGTGTACCGACTATTGGGGTCAGGGCACACAAGTC
ACGGTCTCAAGCGGCGGAAGCTCTGGTGAACAGAAACTGATTTCAGAAG
DCHVLWR#LDGLF#LPSVCT#evqlvesggglvqagdslrlscaa
sgDCHVLWRmgwfrqapgkerefvaaisLDGLFtnyadsvrgrfsisads
akntvylqmnslkpedtavyycaaLPSVCTaywgqgtqvtvss#AGGAGA
TATACCATGGAAGTTCAACTGGTTGAATCTGGTGGCGGTTTAGTTCAAGC
CGGTGATAGCCTTCGTCTGAGCTGCGCCGCAAGCGGAGATTGTCACGTTC
TCTGGCGTATGGGTTGGTTTCGCCAGGCACCTGGTAAAGAACGTGAATTT
GTGGCAGCCATTAGCCTCGACGGTTTGTTTACCAACTATGCCGATTCTGT
TCGTGGTCGCTTCAGCATTTCAGCAGATTCTGCGAAGAATACCGTGTACT
TACAGATGAACTCACTGAAACCTGAGGATACCGCCGTTTATTATTGTGCG
GCACTGCCTAGTGTGTGTACCGCCTATTGGGGTCAGGGCACACAAGTCAC
GGTCTCAAGCGGCGGAAGCTCTGGTGAACAGAAACTGATTTCAGAAG
0
DCHVLWR#LDGLF#LPSVCT#evqlvesggglvqagdslrlscaasg
DCHVLWRmgwfrqapgkeresvaaisLDGLFtnyadsvrgrfsisadsak
ntvylqmnslkpedtavyycaaLPSVCTaywgqgtqvtvss#GGAGATAT
ACCATGGAAGTTCAACTGGTTGAATCTGGTGGCGGTTTAGTTCAAGCCGG
TGATAGCCTTCGTCTGAGCTGCGCCGCAAGCGGAGATTGTCACGTTCTCT
GGCGTATGGGTTGGTTTCGCCAGGCACCTGGTAAAGAACGTGAATCTGTG
GCAGCCATTAGCCTCGACGGTTTGTTTACCAACTATGCCGATTCTGTTCG
TGGTCGCTTCAGCATTTCAGCAGATTCTGCGAAGAATACCGTGTACTTAC
AGATGAACTCACTGAAACCTGAGGATACCGCCGTTTATTATTGTGCGGCA
CTGCCTAGTGTGTGTACCGCCTATTGGGGTCAGGGCACACAAGTCACGGT
CTCAAGCGGCGGAAGCTCTGGTGAACAGAAACTGATTTCAGAAG
DCHVLWR#LDGLF#MPFF#evqlvesggglvqagdslrlscaasgDCHVL
WRmgwfrqapgkereflaaisLDGLFtnyadsvrgrfsisadsakntvyl
qmnslkpedtavyycaaMPFFdywgqgtqvtvss#ATACCATGGAAGTTC
AACTGGTTGAATCTGGTGGCGGTTTAGTTCAAGCCGGTGATAGCCTTCGT
CTGAGCTGCGCCGCAAGCGGAGATTGTCACGTTCTCTGGCGTATGGGTTG
GTTTCGCCAGGCACCTGGTAAAGAACGTGAATTTTTGGCAGCCATTAGCC
TCGACGGTTTGTTTACCAACTATGCCGATTCTGTTCGTGGTCGCTTCAGC
ATTTCAGCAGATTCTGCGAAGAATACCGTGTACTTACAGATGAACTCACT
GAAACCTGAGGATACCGCCGTTTATTATTGTGCGGCAATGCCTTTTTTTG
ACTATTGGGGTCAGGGCACACAAGTCACGGTCTCAAGCGGCGGAAGCTCT
GGTGAACAGAAACTGATTTCAGAAG
DCHVLWR#LDGLF#HHFHP
GGMD#evqlvesggglvqagdslrlscaasgDCHVLWRmgwfrqapgker
efvaaisLDGLFtnyadsvrgrfsisadsakntvylqmnslkpedtavyy
cnaHHFHPGGMDdywgqgtqvtvsi#ATATACCATGGAAGTTCAACTGGT
TGAATCTGGTGGCGGTTTAGTTCAAGCCGGTGATAGCCTTCGTCTGAGCT
GCGCCGCAAGCGGAGATTGTCACGTTCTCTGGCGTATGGGTTGGTTTCGC
CAGGCACCTGGTAAAGAACGTGAATTTGTGGCAGCCATTAGCCTCGACGG
TTTGTTTACCAACTATGCCGATTCTGTTCGTGGTCGCTTCAGCATTTCAG
CAGATTCTGCGAAGAATACCGTGTACTTACAGATGAACTCACTGAAACCT
GAGGATACCGAACAGAAACTGATTTCAGAAG
DCHVLWR#LDGLFMPFF#qvqlvesggalvqpggslrlscaasgDCHVLW
Rmgwfrqapgke
refvaaisLDGLFtnyadsvrgrfsisadsakntvylqmnslkpedtavy
ycaaMPFFaywgqgtqvtvss#ATACCATGCAGGTTCAACTGGTTGAATC
TGGTGGCGCATTAGTTCAACCGGGTGGTAGCCTTCGTCTGAGCTGCGCCG
CAAGCGGAGATTGTCACGTTCTCTGGCGTATGGGTTGGTTTCGCCAGGCA
CCTGGTAAAGAACGTGAATTTGTGGCAGCCATTAGCCTCGACGGTTTGTT
TACCAACTATGCCGATTCTGTTCGTGGTCGCTTCAGCATTTCAGCAGATT
CTGCGAAGAATACCGTGTACTTACAGATGAACTCACTGAAACCTGAGGAT
ACCGCCGTTTATTATTGTGCGGCAATGCCTTTTTTTGCCTATTGGGGTCA
GGGCACACAAGTCACGGTCTCAAGCGGCGGAAGCTCTGGTGAACAGAAAC
TGATTTCAGAAG
DCHVLWR#LDGLF#MPFF#evqlvesggglvq
agdslrlscaasgDCHVLWRmgwfrqapgkerefvaaisLDGLFtnyads
vrgrfsisadsakntvylqmnsqkpedtavyycaaMPTTGTCACGTTCTC
TGGCGTATGGGTTGGTTTCGCCAGGCACCTGGTAAAGAACGTGAATTTGT
GGCAGCCATTAGCCTCGACGGTTTGTTTACCAACTATGCCGATTCTGTTC
GTGGTCGCTTCAGCATTTCAGCAGATTCTGCGAAGAATACCGTGTACTTA
CAGATGAACTCACAGAAACCTGAGGATACCGCCGTTTATTATTGTGCGGC
AATGCCTTTTTTTGACTATTGGGGTCAGGGCACACAAGTCACGGTCTCAA
GCGGCGGAAGCTCTGGTGAACAGAAACTGATTTCAGAAG
DCHVL
WR#LDGLF#LPVTSG#evqlvesggglvqagdslrlscaasgDCHVLWRm
gwfrqapgkerefvaaisLDGLFtnyadsvrgrfsisadsakntvylqmn
slkpedtavyycaaLPVTSGdywgqgtqvtvss#ACCATGGAAGTTCAAC
TGGTTGAATCTGGTGGCGGTTTAGTTCAAGCCGGTGATAGCCTTCGTCTG
AGCTGCGCCGCAAGCGGAGATTGTCACGTTCTCTGGCGTATGGGTTGGTT
TCGCCAGGCACCTGGTAAAGAACGTGAATTTGTGGCAGCCATTAGCCTCG
ACGGTTTGTTTACCAACTATGCCGATTCTGTTCGTGGTCGCTTCAGCATT
TCAGCAGATTCTGCGAAGAATACCGTGTACTTACAGATGAACTCACTGAA
ACCTGAGGATACCGCCGTTTATTATTGTGCGGCATTGCCGGTGACTTCTG
GCGACTATTGGGGTCAGGGCACACAAGTCACGGTCTCAAGCGGCGGAAGC
TCTGGTGAACAGAAACTGATGTCAGAAG
DCHVLWR#LDGLF#MP
FF#evqlvesggglvqagdslrlscaasgDCHVLWRmgwfrqapgkeref
vaaisLDGLFtnyadfvrgrfsisadsakntvylqmnslkpedtavyyca
aMPFFdywgqgtqvtvss#AAGGAGATATACCATGGAAGTTCAACTGGTT
GAATCTGGTGGCGGTTTAGTTCAAGCCGGTGATAGCCTTCGTCTGAGCTG
CGCCGCAAGCGGAGATTGTCACGTTCTCTGGCGTATGGGTTGGTTTCGCC
AGGCACCTGGTAAAGAACGTGAATTTGTGGCAGCCATTAGCCTCGACGGT
TTGTTTACCAACTATGCCGATTTTGTTCGTGGTCGCTTCAGCATTTCAGC
AGATTCTGCGAAGAATACCGTGTACTTACAGATGAACTCACTGAAACCTG
AGGATACCGCCGTTTATTATTGTGCGGCAATGCCTTTTTTTGACTATTGG
GGTCAGGGCACACAAGTCACGGTCTCAAGCGGCGGAAGCTCTGGTGAACA
GAAACTGATTTCAGAAG
DCHVLWR#LDGLF#MPFF#evqlvesg
gglvqagdslrlscaasgDCHVLWRmgwfrqapgkerefvaairLDGLFt
nyadsvrgrfsisadsakntvylqmnslkpedtavyycaaMPFFdywgqg
tqvtvss#ACCATGGAAGTTCAACTGGTTGAATCTGGTGGCGGTTTAGTT
CAAGCCGGTGATAGCCTTCGTCTGAGCTGCGCCGCAAGCGGAGATTGTCA
CGTTCTCTGGCGTATGGGTTGGTTTCGCCAGGCACCTGGTAAAGAACGTG
AATTTGTGGCAGCCATTAGGCTCGACGGTTTGTTTACCAACTATGCCGAT
TCTGTTCGTGGTCGCTTCAGCATTTCAGCAGATTCTGCGAAGAATACCGT
GTACTTACAGATGAACTCACTGAAACCTGAGGATACCGCCGTTTATTATT
GTGCGGCAATGCCTTTTTTTGACTATTGGGGTCAGGGCACACAAGTCACG
GTCTCAAGCGGCGGAAGCTCTGGTGAACAGAAACTGATTTCAGAAG
DCHVLWR#LDGLF#MPFF#evqlvesggglvqagdslrlscaasgDCH
VLWRmgwfrqapgkerefvaaisLDGLFtnyadsvrgrcsisadsakntv
ylqmnslkpedtavyycaaMPFFdywgqgtqvtvss#GGAGATATACCAT
GGAAGTTCAACTGGTTGAATCTGGTGGCGGTTTAGTTCAAGCCGGTGATA
GCCTTCGTCTGAGCTGCGCCGCAAGCGGAGATTGTCACGTTCTCTGGCGT
ATGGGTTGGTTTCGCCAGGCACCTGGTAAAGAACGTGAATTTGTGGCAGC
CATTAGCCTCGACGGTTTGTTTACCAACTATGCCGATTCTGTTCGTGGTC
GCTGCAGCATTTCAGCAGATTCTGCGAAGAATACCGTGTACTTACAGATG
AACTCACTGAAACCTGAGGATACCGCCGTTTATTATTGTGCGGCAATGCC
TTTTTTTGACTATTGGGGTCAGGGCACACAAGTCACGGTCTCAAGCGGCG
GAAGCTCTGGTGAACAGAAACTGATTTCAGAAG
DCHVLWR#LDG
LF#LPSTPECYAL#evqlvesggglvqagdslrlscaasgDCHVLWRmgw
frqapgkerefvaaisLDGLFtnyadsvrgrfsisadsakntvylqmnsl
kpedtavyycAGCGGAGATTGTCACGTTCTCTGGCGTATGGGTTGGTTTC
GCCAGGCACCTGGTAAAGAACGTGAATTTGTGGCAGCCATTAGCCTCGAC
GGTTTGTTTACCAACTATGCCGATTCTGTTCGTGGTCGCTTCAGCATTTC
AGCAGATTCTGCGAAGAATACCGTGTACTTACAGATGAACTCACTGAAAC
CTGAGGATACCGCCGTTTATTATTGTGCGACACTGCCTAGCACCCCTGAG
TGTTATGCCTTGGGCTATTGGGGTCAGGACACACAAGTCACGGTCTCAAG
CGGCGGAAGCTCTGGTGAACAGAAACTGATTTCAGAAG
DCHVLW
R#LDGLF#MPFF#evqlvesggglvhagdrlrlscaasgDCHVLWRmgwf
rqapgkerefvaaisLDGLFtnyadsvrgrfsisadsakntvylqmnslk
pedtavyycaaMPFFdywgqgtqvtvss#GAAGGAGATATACCATGGAAG
TTCAACTGGTTGAATCTGGTGGCGGTTTAGTTCACGCCGGTGATCGCCTT
CGTCTGAGCTGCGCCGCAAGCGGAGATTGTCACGTTCTCTGGCGTATGGG
TTGGTTTCGCCAGGCACCTGGTAAAGAACGTGAATTTGTGGCAGCCATTA
GCCTCGACGGTTTGTTTACCAACTATGCCGATTCTGTTCGTGGTCGCTTC
AGCATTTCAGCAGATTCTGCGAAGAATACCGTGTACTTACAGATGAACTC
ACTGAAACCTGAGGATACCGCCGTTTATTATTGTGCGGCAATGCCTTTTT
TTGACTATTGGGGTCAGGGCACACAAGTCACGGTCTCAAGCGGCGGAAGC
TCTGGTGAACAGAAACTGATTTCAGAAG
DCHVLWR#LDGLF#HH
FHPGGMD#evqlvesggglvqagdslrlscdasgDCHVLWRmgwfrqapg
kerefvaaisLDGLFtnyadsvrgrfsisadsakntvylqmnslkpedta
vyycnaHHFHPGGMDdywgqgtqvtvss#GGAGATATACCATGGAAGTTC
AACTGGTTGAATCTGGTGGCGGTTTAGTTCAAGCCGGTGATAGCCTTCGT
CTGAGCTGCGACGCAAGCGGAGATTGTCACGTTCTCTGGCGTATGGGTTG
GTTTCGCCAGGCACCTGGTAAAGAACGTGAATTTGTGGCAGCCATTAGCC
TCGACGGTTTGTTTACCAACTATGCCGATTCTGTTCGTGGTCGCTTCAGC
ATTTCAGCAGATTCTGCGAAGAATACCGTGTACTTACAGATGAACTCACT
GAAACCTGAGGATACCGCCGTTTATTATTGTAATGCACACCATTTTCATC
CTGGTGGTATGGATGACTACTGGGGTCAGGGCACACAAGTCACGGTCTCA
AGCGGCGGAAGCTCTGGTGAACAGAAACTGATTTCAGAAG
DCHV
LWR#LDGLF#LPATSG#evqlvdsggglvhagdrlrlscaasgDCHVLWR
mgwfrqapgkerefvaaisLDGLFtnyadsvrgrfsisadsakntvylqm
nslkpedtavyycaaLPATSGdywgqgtqvtvss#GAAGGAGATATACCA
TGGAAGTTCAACTGGTTGACTCTGGTGGCGGTTTAGTTCACGCCGGTGAT
CGCCTTCGTCTGAGCTGCGCCGCAAGCGGAGATTGTCACGTTCTCTGGCG
TATGGGTTGGTTTCGCCAGGCACCTGGTAAAGAACGTGAATTTGTGGCAG
CCATTAGCCTCGACGGTTTGTTTACCAACTATGCCGATTCTGTTCGTGGT
CGCTTCAGCATTTCAGCAGATTCTGCGAAGAATACCGTGTACTTACAGAT
GAACTCACTGAAACCTGAGGATACCGCCGTTTATTATTGTGCGGCATTGC
CGGCGACTTCTGGCGACTATTGGGGTCAGGGCACACAAGTCACGGTCTCA
AGCGGCGGAAGCTCTGGTGAACAGAAACTGATTTCAGAAG
DCHV
LWR#LDGLF#LPATSG#evqlvesggglvqagdslrlscaangDCHVLWR
mgwfrqapgkerefvaaisLDGLFtnyadsvrgrfsisadsakntvylqi
nslkpedtavyycaaLPATSGdywvqgtqvtvss#ATACCATGGAAGTTC
AACTGGTTGAATCTGGTGGCGGTTTAGTTCAAGCCGGTGATAGCCTTCGT
CTGAGCTGCGCCGCAAACGGAGATTGTCACGTTCTCTGGCGTATGGGTTG
GTTTCGCCAGGCACCTGGTAAAGAACGTGAATTTGTGGCAGCCATTAGCC
TCGACGGTTTGTTTACCAACTATGCCGATTCTGTTCGTGGTCGCTTCAGC
ATTTCAGCAGATTCTGCGAAGAATACCGTGTACTTACAGATTAACTCACT
GAAACCTGAGGATACCGCCGTTTATTATTGTGCGGCATTGCCGGCGACTT
CTGGCGACTATTGGGTTCAGGGCACACAAGTCACGGTCTCAAGCGGCGGA
AGCTCTGGTGAACAGAAACTGCTTTCAGAAG
DCHVLWR#LDGLFMPFF#evqlvesggglvqagdslrlscaangDCHVLW
Rmgwfrqapgke
refvaaisLDGLFtnyadsvrgrfsisadsakntvylqmnslkpedtavy
ycaaMPFFdywgqgtqvtvss#GAAGGAGATATACCATGGAAGTTCAACT
GGTTGAATCTGGTGGCGGTTTAGTTCAAGCCGGTGATAGCCTTCGTCTGA
GCTGCGCCGCAAACGGAGATTGTCACGTTCTCTGGCGTATGGGTTGGTTT
CGCCAGGCACCTGGTAAAGAACGTGAATTTGTGGCAGCCATTAGCCTCGA
CGGTTTGTTTACCAACTATGCCGATTCTGTTCGTGGTCGCTTCAGCATTT
CAGCAGATTCTGCGAAGAATACCGTGTACTTACAGATGAACTCACTGAAA
CCTGAGGATACCGCCGTTTATTATTGTGCGGCAATGCCTTTTTTTGACTA
TTGGGGTCAGGGCACACAAGTCACGGTCTCAAGCGGCGGAAGCTCTGGTG
AACAGAAACTGATTTCAGAAG
DCHVLWR#LDGLF#HHFHPGGMDevqlvesggglvqvgdslrlscaasgD
CHVLWRmgwfrqapgkerefva
aisLDGLFtnyadsvrgrfsisadsakntvylqmnslkpedtavyycnaH
HFHPGGMDdywgqgtqvtvss#AAGGAGATATACCATGGAAGTTCAACTG
GTTGAATCTGGTGGCGGTTTAGTTCAAGTCGGTGATAGCCTTCGTCTGAG
CTGCGCCGCAAGCGGAGATTGTCACGTTCTCTGGCGTATGGGTTGGTTTC
GCCAGGCACCTGGTAAAGAACGTGAATTTGTGGCAGCCATTAGCCTCGAC
GGTTTGTTTACCAACTATGCCGATTCTGTTCGTGGTCGCTTCAGCATTTC
AGCAGATTCTGCGAAGAATACCGTGTACTTACAGATGAACTCACTGAAAC
CTGGCTCTGGTGAACAGAAACTGATTTCAGAAG
DCHVLWR#LDG
LF#HHFHPGGMD#evqlvesggglvqagdslrlscaasgDCHVLWRmgwf
rqapgkerefvaaisLDGLFtnyadsvrgrfsisadsakntvylqmnslk
pedtavyycnaHHFHPGGMDdyggqgtqvtvss#AGATATACCATGGAAG
TTCAACTGGTTGAATCTGGTGGCGGTTTAGTTCAAGCCGGTGATAGCCTT
CGTCTGAGCTGCGCCGCAAGCGGAGATTGTCACGTTCTCTGGCGTATGGG
TTGGTTTCGCCAGGCACCTGGTAAAGAACGTGAATTTGTGGCAGCCATTA
GCCTCGACGGTTTGTTTACCAACTATGCCGATTCTGTTCGTGGTCGCTTC
AGCATTTCAGCAGATTCTGCGAAGAATACCGTGTACTTACAGATGAACTC
ACTGAAACCTGAGGATACCGCCGTTTATTATTGTAATGCACACCATTTTC
ATCCTGGTGGTATGGATGACTATGGGGGGCAGGGCACACAAGTCACGGTC
TCAAGCGGCGGAAGCTCTGGTGAACAGAAACTGATTTCAGAAG
D
CHVLWR#LDGLF#MPFF#evqlvesggglvqagdslrlscaasgDCHVLW
RmgwfrqapgkerefvaaisLDGLFtnyadsvrgrfsisadsakntvylq
mnslkpedtavyycaaMPFFdyggqgtqvtvas#ATACCATGGAAGTTCA
ACTGGTTGAATCTGGTGGCGGTTTAGTTCAAGCCGGTGATAGCCTTCGTC
TGAGCTGCGCCGCAAGCGGAGATTGTCACGTTCTCTGGCGTATGGGTTGG
TTTCGCCAGGCACCTGGTAAAGAACGTGAATTTGTGGCAGCCATTAGCCT
CGACGGTTTGTTTACCAACTATGCCGATTCTGTTCGTGGTCGCTTCAGCA
TTTCAGCAGATTCTGCGAAGAATACCGTGTACTTACAGATGAACTCACTG
AAACCTGAGGATACCGCCGTTTATTATTGTGCGGCAATGCCTTTTTTTGA
CTATGGGGGTCAGGGCACACAAGTCACGGTCGCAAGCGGCGGAAGCTCTG
GGGAACAGAAACTGATTTCAGAAG
DCHVLWR#LDGLF#LPSVCTevqlvesggdlvqagdslrlscaasgDCHV
LWRmgwfrqapgkerefva
aisLDGLFtnyadsvrgrfsisadsakntvylqmnslkpedtavyycaaL
PSVCTdywgqgtqvtvss#GGAGATATACCATGGAAGTTCAACTGGTTGA
ATCTGGTGGCGATTTAGTTCAAGCCGGTGATAGCCTTCGTCTGAGCTGCG
CCGCAAGCGGAGATTGTCACGTTCTCTGGCGTATGGGTTGGTTTCGCCAG
GCACCTGGTAAAGAACGTGAATTTGTGGCAGCCATTAGCCTCGACGGTTT
GTTTACCAACTATGCCGATTCTGTTCGTGGTCGCTTCAGCATTTCAGCAG
ATTCTGCGAAGAATACCGTGTACTTACAGATGAACTCACTGAAACCTGAG
GATACCGCCGTTTATTATTGTGCGGCACTGCCTAGTGTGTGTACCGACTA
TTGGGGTCAGGGCACACAAGTCACGGTCTCAAGCGGCGGAAGCTCTGGTG
AACAGAAACTGATTTCAGAAG
DCHVLWR#LDGLF#LPATSG#ev
qlvesggglvqagdslrlscaasgDCHVLWRmgwfrqapgkerefvaais
LDGLFtnyadsvrgrfsisadsakntvylqmnslkpedtavyycaaLPAT
SGdyggqgtqvtvas#ACCATGGAAGTTCAACTGGTTGAATCTGGTGGCG
GTTTAGTTCAAGCCGGTGATAGCCTTCGTTTGAGCTGCGCCGCAAGCGGA
GATTGTCACGTTCTCTGGCGTATGGGTTGGTTTCGCCAGGCACCTGGTAA
AGAACGTGAATTTGTGGCAGCCATTAGCCTCGACGGTTTGTTTACCAACT
ATGCCGATTCTGTTCGTGGTCGCTTCAGCATTTCAGCAGATTCTGCGAAG
AATACCGTGTACTTACAGATGAACTCACTGAAACCTGAGGATACCGCCGT
TTATTATTGTGCGGCATTGCCGGCGACTTCTGGCGACTATGGGGGTCAGG
GCACACAAGTCACGGTCGCAAGCGGCGGAAGCTCTGGGGAACAGAAACTG
ATTTCAGAAG
DCHVLWR#LDGFF#LPSVCT#evqlvesggglvq
agdslrlscaasgDCHVLWRmgwfrqapgkerefvaaisLDGFFtnyads
vrgrfsisadsakntvylqmnslkpedtavyycaaLPSVCTdywgqgtqv
tvss#AGGAGATATACCATGGAAGTTCAACTGGTTGAATCTGGTGGCGGT
TTAGTTCAAGCCGGTGATAGCCTTCGTCTGAGCTGCGCCGCAAGCGGAGA
TTGTCACGTTCTCTGGCGTATGGGTTGGTTTCGCCAGGCACCTGGTAAAG
AACGTGAATTTGTGGCAGCCATTAGCCTCGACGGTTTTTTTACCAACTAT
GCCGATTCTGTTCGTGGTCGCTTCAGCATTTCAGCAGATTCTGCGAAGAA
TACCGTGTACTTACAGATGAACTCACTGAAACCTGAGGATACCGCCGTTT
ATTATTGTGCGGCACTGCCTAGTGTGTGTACCGACTATTGGGGTCAGGGC
ACACAAGTCACGGTCTCAAGCGGCGGAAGCTCTGGTGAACAGAAACTGAT
TTCAGAAG
DCHVLWR#LDGLF#LPSTPECYAL#evqlvesgggl
vqagdslrlscaasgDCHVLWRmgwfrqapgkerefvaaisLDGLFtnya
dsvrgrfsisadsakntvylqmnslkpedtavyycatLPSTPECYALgyw
gqgtqvtvss#AGGAGATATACCATGGAAGTTCAACTGGTTGAATCTGGT
GGCGGTTTAGTTCAAGCCGGTGATAGCCTTCGTCTGAGCTGCGCCGCAAG
CGGAGATTGTCACGTTCTCTGGCGTATGGGTTGGTTTCGCCAGGCACCTG
GTAAAGAACGTGAATTTGTGGCAGCCATTAGCCTCGACGGTTTGTTTACC
AACTATGCCGATTCTGTTCGTGGTCGCTTCAGCATTTCAGCAGATTCTGC
GAAGAATACCGTGTACTTACAGATGAACTCACTGAAACCTGAGGATACCG
CCGTTTATTATTGTGCGACACTGCCTAGCACCCCTGAGTGTTATGCCTTG
GGCTATTGGGGTCAGGGCACACAAGTCACGGTCTCAAGCGGCGGAAGCTC
TGGTGAACAGAAACTGATTTCAGAAG
DCHVLWR#LDGLF#MPFFevqlvesggglvqagdslrlscaasgDCHVLW
Rmgwfrqapgkerefva
aisLDGLFtnyadsvrgrfsisadsakntvclqmnslkpedtavyycaaM
PFFdywgqgtqvtvss#AAGGAGATATACCATGGAAGTTCAACTGGTTGA
ATCTGGTGGCGGTTTAGTTCAAGCCGGTGATAGCCTTCGTCTGAGCTGCG
CCGCAAGCGGAGATTGTCACGTTCTCTGGCGTATGGGTTGGTTTCGCCAG
GCACCTGGTAAAGAACGTGAATTTGTGGCAGCCATTAGCCTCGACGGTTT
GTTTACCAACTATGCCGATTCTGTTCGTGGTCGCTTCAGCATTTCAGCAG
ATTCTGCGAAGAATACCGTGTGCTTACAGATGAACTCACTGAAACCTGAG
GATACCGCCGTTTATTATTGTGCGGCAATGCCTTTTTTTGACTATTGGGG
TCAGGGCACACAAGTCACGGTCTCAAGCGGCGGAAGCTCTGGTGAACAGA
AACTGATTTCAGAAG
DCHVLWR#LDGLF#MSQPTL#evqlvesg
gglvqagdslrlscaasgDCHVLWRmgwfrqapgkerefvaaisLDGLFt
nyadsvrgrfsisadsakntvylqmnslkpedtavyycaaMSQPTLdywg
qgtqvtvss#ATATACCATGGAAGTTCAACTGGTTGAATCTGGTGGCGGT
TTAGTTCAAGCCGGTGATAGCCTTCGTCTGAGCTGCGCCGCAAGCGGAGA
TTGTCACGTTCTCTGGCGTATGGGTTGGTTTCGCCAGGCACCTGGTAAAG
AACGTGAATTTGTGGCAGCCATTAGCCTCGACGGTTTGTTTACCAACTAT
GCCGATTCTGTTCGTGGTCGCTTCAGCATTTCAGCAGATTCTGCGAAGAA
TACCGTGTACTTACAGATGAACTCACTGAAACCTGAGGATACCGCCGTTT
ATTATTGTGCGGCAATGAGTCAGCCTACCTTGGACTATTGGGGTCAGGGC
ACACAAGTCACGGTCTCAAGCGGCGGAAGCTCTGGTGAACAGAAACTGAT
TTCAGAAG
DCHVLWR#LDGLF#MPFF#evqlvesggglvqagds
lrIncaasgDCHVLWRmgwfrqapgkerefvaaisLDGLFtnyadsvrgr
fsisadsakntvylqmnslkpedtavyycaaMPFFdywgqgtqvtvss#G
AAGGAGATATACCATGGAAGTTCAACTGGTTGAATCTGGTGGCGGTTTAG
TTCAAGCCGGTGATAGCCTTCGTCTGAACTGCGCCGCAAGCGGAGATTGT
CACGTTCTCTGGCGTATGGGTTGGTTTCGCCAGGCACCTGGTAAAGAACG
TGAATTTGTGGCAGCCATTAGCCTCGACGGTTTGTTTACCAACTATGCCG
ATTCTGTTCGTGGTCGCTTCAGCATTTCAGCAGATTCTGCGAAGAATACC
GTGTACTTACAGATGAACTCACTGAAACCTGAGGATACCGCCGTTTATTA
TTGTGCGGCAATGCCTTTTTTTGACTATTGGGGTCAGGGCACACAAGTCA
CGGTCTCAAGCGGCGGAAGCTCTGGTGAACAGAAACTGATTTCAGAAG
DCHVLWR#LDGLF#MPFF#evqlvesggglvqagdslrlscaasgD
CHVLWRmgwfrqapgkerefvaaisLDGLFtnyadsvrgrfsisadsakn
tvylqmnsInpedtavyycaaMPFFdywgqgtqvtvss#AGATATACCAT
GGAAGTTCAACTGGTTGAATCTGGTGGCGGTTTAGTTCAAGCCGGTGATA
GCCTTCGTCTGAGCTGCGCCGCAAGCGGAGATTGTCACGTTCTCTGGCGT
ATGGGTTGGTTTCGCCAGGCACCTGGTAAAGAACGTGAATTTGTGGCAGC
CATTAGCCTCGACGGTTTGTTTACCAACTATGCCGATTCTGTTCGTGGTC
GCTTCAGCATTTCAGCAGATTCTGCGAAGAATACCGTGTACTTACAGATG
AACTCACTGAACCCTGAGGATACCGCCGTTTATTATTGTGCGGCAATGCC
TTTTTTTGACTATTGGGGTCAGGGCACACAAGTCACGGTCTCAAGCGGCG
GAAGCTCTGGTGAACAGAAACTGATTTCAGAAG
DCQVLWR#LDG
LF#MPFF#evqlvesggglvqagdslrlscaasgDCQVLWRmgwfrqapg
kerefvaaisLDGLFtnyadsvrgrfsisadsakntvylqmnslkpedta
vyycaaMPFFdywgqgtqvtvss#AGGAGATATACCATGGAAGTTCAACT
GGTTGAATCTGGTGGCGGTTTAGTTCAAGCCGGTGATAGCCTTCGTCTGA
GCTGCGCCGCAAGCGGAGATTGTCAAGTTCTCTGGCGTATGGGTTGGTTT
CGCCAGGCACCTGGTAAAGAACGTGAATTTGTGGCAGCCATTAGCCTCGA
CGGTTTGTTTACCAACTATGCCGATTCTGTTCGTGGTCGCTTCAGCATTT
CAGCAGATTCTGCGAAGAATACCGTGTACTTACAGATGAACTCACTGAAA
CCTGAGGATACCGCCGTTTATTATTGTGCGGCAATGCCTTTTTTTGACTA
TTGGGGTCAGGGCACACAAGTCACGGTCTCAAGCGGCGGAAGCTCTGGTG
AACAGAAACTGATTTCAGAAG
DCHVLWR#LDGLF#MPFF#evql
vesggglvqagdslrlscaasgDCHVLWRmgwfrqapgkerefvaaisLD
GLFtnyadsvrgrfsisadsakntvylqmnslkpedtavyycaaMPFFdy
wcqgtqvtvss#GAAGGAGATATACCATGGAAGTTCAACTGGTTGAATCT
GGTGGCGGTTTAGTTCAAGCCGGTGATAGCCTTCGTCTGAGCTGCGCCGC
AAGCGGAGATTGTCACGTTCTCTGGCGTATGGGTTGGTTTCGCCAGGCAC
CTGGTAAAGAACGTGAATTTGTGGCAGCCATTAGCCTCGACGGTTTGTTT
ACCAACTATGCCGATTCTGTTCGTGGTCGCTTCAGCATTTCAGCAGATTC
TGCGAAGAATACCGTGTACTTACAGATGAACTCACTGAAACCTGAGGATA
CCGCCGTTTATTATTGTGCGGCAATGCCTTTTTTTGACTATTGGTGTCAG
GGCACACAAGTCACGGTCTCAAGCGGCGGAAGCTCTGGTGAACAGAAACT
GATTTCAGAAG
DCRVLWR#LDGLF#LPATSG#evqlvesggglv
qagdslrlscaasgDCRVLWRmgwfrqapgkerefvaaisLDGLFtnyad
svrgrfsisadsakntvylqmnslkpedtavyycaaLPATSGdywgqgtq
vtvss#AGATATACCATGGAAGTTCAACTGGTTGAATCTGGTGGCGGTTT
AGTTCAAGCCGGTGATAGCCTTCGTCTGAGCTGCGCCGCAAGCGGAGATT
GTCGCGTTCTCTGGCGTATGGGTTGGTTTCGCCAGGCACCTGGTAAAGAA
CGTGAATTTGTGGCAGCCATTAGCCTCGACGGTTTGTTTACCAACTATGC
CGATTCTGTTCGTGGTCGCTTCAGCATTTCAGCAGATTCTGCGAAGAATA
CCGTGTACTTACAGATGAACTCACTGAAACCTGAGGATACCGCCGTTTAT
TATTGTGCGGCATTGCCGGCGACTTCTGGCGACTATTGGGGTCAGGGCAC
ACAAGTCACGGTCTCAAGCGGCGGAAGCTCTGGTGAACAGAAACTGATTT
CAGAAG
DCHVLWR#LDGLF#HHFHPGGMD#evqlfesggglvqa
gdslrlscaasgDCHVLWRmgwfrqapgkerefvaaisLDGLFtnyadsv
rgrfsisadsakntvylqmnslkpedtavyycnaHHFHPGGMDdywgqgt
qvtvss#AGATATACCATGGAAGTTCAACTGTTTGAATCTGGTGGCGGTT
TAGTTCAAGCCGGTGATAGCCTTCGTCTGAGCTGCGCCGCAAGCGGAGAT
TGTCACGTTCTCTGGCGTATGGGTTGGTTTCGCCAGGCACCTGGTAAAGA
ACGTGAATTTGTGGCAGCCATTAGCCTCGACGGTTTGTTTACCAACTATG
CCGATTCTGTTCGTGGTCGCTTCAGCATTTCAGCAGATTCTGCGAAGAAT
ACCGTGTACTTACAGATGAACTCACTGAAACCTGAGGATACCGCCGTTTA
TTATTGTAATGCACACCATTTTCATCCTGGTGGTATGGATGACTATTGGG
GTCAGGGCACACAAGTCACGGTCTCAAGCGGCGGAAGCTCTTGTGAACAG
AAACTGATTTCAGAAG
DCHVLWR#LDGLF#MPFF#evqlvesgg
glvqagdslrlscaasgDCHVLWRmgwfrqapgkerefvaaisLDGLFtn
yadsvrgrfsisadsakntvylqmnsrkpedtavyycaaMPFFdywgqgt
qvtvss#ATACCATGGAAGTTCAACTGGTTGAATCTGGTGGCGGTTTAGT
TCAAGCCGGTGATAGCCTTCGTCTGAGCTGCGCCGCAAGCGGAGATTGTC
ACGTTCTCTGGCGTATGGGTTGGTTTCGCCAGGCACCTGGTAAAGAACGT
GAATTTGTGGCAGCCATTAGCCTCGACGGTTTGTTTACCAACTATGCCGA
TTCTGTTCGTGGTCGCTTCAGCATTTCAGCAGATTCTGCGAAGAATACCG
TGTACTTACAGATGAACTCACGGAAACCTGAGGATACCGCCGTTTATTAT
TGTGCGGCAATGCCTTTTTTTGACTATTGGGGTCAGGGCACACAAGTCAC
GGTCTCAAGCGGCGGAAGCTCTGGTGAACAGAAACTGATTTCAGAAG
0
DCHVLWR#LDGLF#LPATSG#evrlvesggglvqagdslrlscaasg
DCHVLWRmgwfrqapgkerefvaaisLDGLFtnyadsvrgrfsisadsak
ntvylqmnslkpedtavyycatLPATSGdywgqgtqvtvss#AGGAGATA
TACCATGGAAGTTCGACTGGTTGAATCTGGTGGCGGTTTAGTTCAAGCCG
GTGATAGCCTTCGTCTGAGCTGCGCCGCAAGCGGAGATTGTCACGTTCTC
TGGCGTATGGGTTGGTTTCGCCAGGCACCTGGTAAAGAACGTGAATTTGT
GGCAGCCATTAGCCTCGACGGTTTGTTTACCAACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGAC
ATTGCCGGCGACTTCTGGCGACTATTGGGGTCAGGGCACACAAGTCACGG
TCTCAAGCGGCGGAAGCTCTGGTGAACAGAAACTGATTTCAGAAG
DCHVLWR#LDGLF#MPFF#evqlvesggglvqdgdslrlscaasgDCHV
LWRmgwfrqapgkerefvaaisLDGLFtnyadsvrgrfsisadsakntvy
lqmnslkpedtavyycaaMPFFdywgqgtqvtvss#ATATACCATGGAAG
TTCAACTGGTTGAATCTGGTGGTGGTTTAGTTCAAGACGGTGATAGCCTT
CGTCTGAGCTGCGCCGCAAGCGGAGATTGTCACGTTCTCTGGCGTATGGG
TTGGTTTCGCCAGGCACCTGGTAAAGAACGTGAATTTGTGGCAGCCATTA
GCCTCGACGGTTTGTTTACCAACTATGCCGATTCTGTTCGTGGTCGCTTC
AGCATTTCAGCAGATTCTGCGAAGAATACCGTGTACTTACAGATGAACTC
ACTGAAACCTGAGGATACCGCCGTTTATTATTGTGCGGCAATGCCTTTTT
TTGACTATTGGGGTCAGGGCACACAAGTCACGGTCTCAAGCGGCGGAAGC
TCTGGTGAACAGAAACTGATTTCAGAAG
DCHVLWR#LDGLF#MP
FF#evqlvesggglvqagdslrlscaasgDCHVLWRmgwfrqapgkeref
vaaisLDGLFtnyadsvrgrfsisadsakntvylqmnslkpedtavyyca
aMPFFdywgqgtqvtass#ATACCATGGAAGTTCAACTGGTTGAATCTGG
TGGCGGTTTAGTTCAAGCCGGTGATAGCCTTCGTCTGAGCTGCGCCGCAA
GCGGAGATTGTCACGTTCTCTGGCGTATGGGTTGGTTTCGCCAGGCACCT
GGTAAAGAACGTGAATTTGTGGCAGCCATTAGCCTCGACGGTTTGTTTAC
CAACTATGCCGATTCTGTTCGTGGTCGCTTCAGCATTTCAGCAGATTCTG
CGAAGAATACCGTGTACTTACAGATGAACTCACTGAAACCTGAGGATACC
GCCGTTTATTATTGTGCGGCAATGCCTTTTTTTGACTATTGGGGTCAGGG
CACACAAGTCACGGCCTCAAGCGGCGGAAGCTTTGGTGAACAGAAACTGA
TTTCAGAAG
DCHVLWR#LDGLF#MPFF#evqlvesggglvqagd
slrlscatsgDCHVLWRmgwfrqapgkerefvaaisLDGLFtnyadsvrg
rfsisadsakntvylqmnslkpedtavyycaaMPFFdywgqgtqvtvss#
AGATATACCATGGAAGTTCAACTGGTTGAATCTGGTGGCGGTTTAGTTCA
AGCCGGTGATAGCCTTCGTCTGAGCTGCGCCACAAGCGGAGATTGTCACG
TTCTCTGGCGTATGGGTTGGTTTCGCCAGGCACCTGGTAAAGAACGTGAA
TTTGTGGCAGCCATTAGCCTCGACGGTTTGTTTACCAACTATGCCGATTC
TGTTCGTGGTCGCTTCAGCATTTCAGCAGATTCTGCGAAGAATACCGTGT
ACTTACAGATGAACTCACTGAAACCTGAGGATACCGCCGTTTATTATTGT
GCGGCAATGCCTTTTTTTGACTATTGGGGTCAGGGCACACAAGTCACGGT
CTCAAGCGGCGGAAGCTCTGGTGAACAGAAACTGATTTCAGAAG
DCHVLWR#LDGLF#MPFF#evqlvdsggglvhagdrlrlscaasgDCHVL
WRmgwfrqapgkerefvaaisLDGLFtnyadsvrgrfsisadsakntvyl
emnslkpedtavyycaaMPFFdywgqgtqvtvss#GAAGGAGATATACCA
TGGAAGTTCAACTGGTTGACTCTGGTGGCGGTTTAGTTCACGCCGGTGAT
CGCCTTCGTCTGAGCTGCGCCGCAAGCGGAGATTGTCACGTTCTCTGGCG
TATGGGTTGGTTTCGCCAGGCACCTGGTAAAGAACGTGAATTTGTGGCAG
CCATTAGCCTCGACGGTTTGTTTACCAACTATGCCGATTCTGTTCGTGGT
CGCTTCAGCATTTCAGCAGATTCTGCGAAGAATACCGTGTACTTAGAGAT
GAACTCACTGAAACCTGAGGATACCGCCGTTTATTATTGTGCGGCAATGC
CTTTTTTTGACTATTGGGGTCAGGGCACACAAGTCACGGTCTCAAGCGGC
GGAAGCTCTGGTGAACAGAAACTGATTTCAGAAG
DCHVLWR#LD
GLF#LPATSG#evqlvesggglvqagdslrlscaasgDCHVLWRmgwfrq
apgkerefvaaisLDGLFtnyadsvrgrfsisadsakntvylqmnslkpe
dtavyycaaLPATSGdywgqdtqvtvss#ATACCATGGAAGTTCAACTGG
TTGAATCTGGTGGCGGTTTAGTTCAAGCCGGTGATAGCCTTCGTCTGAGC
TGCGCCGCAAGCGGAGATTGTCACGTTCTCTGGCGTATGGGTTGGTTTCG
CCAGGCACCTGGTAAAGAACGTGAATTTGTGGCAGCCATTAGCCTCGACG
GTTTGTTTACCAACTATGCCGATTCTGTTCGTGGTCGCTTCAGCATTTCA
GCAGATTCTGCGAAGAATACCGTGTACTTACAGATGAACTCACTGAAACC
TGAGGATACCGCCGTTTATTATTGTGCGGCATTGCCGGCGACTTCTGGCG
ACTATTGGGGTCAGGACACACAAGTCACGGTCTCAAGCGGCGGAAGCTCT
GGTGAACAGAAACTGATTTCAGAAG
DCNVLWR#LDVLF#MPFF#
evqlvesggglvqagdslrlscaasgDCNVLWRmgwfrqapgkerefvaa
isLDVLFtnyadsvrgrfsisadsakntvylqmnslkpedtavyycaaMP
FFdywgqgtqvtvss#AGATATACCATGGAAGTTCAACTGGTTGAATCTG
GTGGCGGTTTAGTTCAAGCCGGTGATAGCCTTCGTCTGAGCTGCGCCGCA
AGCGGAGATTGTAACGTTCTCTGGCGTATGGGTTGGTTTCGCCAGGCACC
TGGTAAAGAACGTGAATTTGTGGCAGCCATTAGCCTCGACGTTTTGTTTA
CCAACTATGCCGATTCTGTTCGTGGTCGCTTCAGCATTTCAGCAGATTCT
GCGAAGAATACCGTGTACTTACAGATGAACTCACTGAAACCTGAGGATAC
CGCCGTTTATTATTGTGCGGCAATGCCTTTTTTTGACTATTGGGGTCAGG
GCACACAAGTCACGGTCTCAAGCGGCGGAAGCTCTGGTGAACAGAAACTG
ATTTCAGAAG
DCHVLWR#LDGLF#MPFF#evqlvesggglvqag
dslrlscaasgDCHVLWRmgwfrqapgkerefvaaisLDGLFtnyadsvr
grfsisadsakntvylqmnslkpegtavyycaaMPFFdywgqgtqvtvss
ACCATGGAAGTTCAACTGGTTGAATCTGGTGGCGGTTTAGTTCAAGCCG
GTGATAGCCTTCGTCTGAGCTGCGCCGCAAGCGGAGATTGTCACGTTCTC
TGGCGTATGGGTTGGTTTCGCCAGGCACCTGGTAAAGAACGTGAATTTGT
GGCAGCCATTAGCCTCGACGGTTTGTTTACCAACTATGCCGATTCTGTTC
GTGGTCGCTTCAGCATTTCAGCAGATTCTGCGAAGAATACCGTGTACTTA
CAGATGAACTCACTGAAACCTGAGGGTACCGCCGTTTATTATTGTGCGGC
AATGCCTTTTTTTGACTATTGGGGTCAGGGCACACAAGTCACGGTCTCAA
GCGGCGGAAGCTCTGGTGAACAGAAACTGATTTCAGAAG
DCHVL
WR#LDGLF#TVPSHNVGGAHVL#evqlvesggglvqagdslrlscaasgD
CHVLWRmgwfrqapgkerefvaaisLDGLFtnyadsvrgrfsisadsakn
tvylqmnslkpedtavyycnaTVPSHNVGGAHVLeywgqgtqvtvss#AT
ACCATGGAAGTTCAACTGGTTGAATCTGGTGGCGGTTTAGTTCAAGCCGG
TGATAGCCTTCGTCTGAGCTGCGCCGCAAGCGGAGATTGTCACGTTCTCT
GGCGTATGGGTTGGTTTCGCCAGGCACCTGGTAAAGAACGTGAATTTGTG
GCAGCCATTAGCCTCGACGGTTTGTTTACCAACTATGCCGATTCTGTTCG
TGGTCGCTTCAGCATTTCAGCAGATTCTGCGAAGAATACCGTGTACTTAC
AGATGAACTCACTGAAACCTGAGGATACCGCCGTTTATTATTGTAATGCA
ACTGTTCCTTCTCATAACGTCGGGGGTGCGCATGTTCTCGAGTATTGGGG
TCAGGGCACACAAGTCACGGTCTCAAGCGGCGGAAGCTCTGGTGAACAGA
AACTGCTTTCAGAAG
DCHVLWR#LDGLF#LPATSG#evqlvesg
gglvqagdslrlscaasgDCHVLWRmdwfrqapgkerefvaaisLDGLFt
nyadsvrgrfsisadsakntvylqmnslkpedtavyycaaLPATSGdywg
qgtqvtvss#ATATACCATGGAAGTTCAACTGGTTGAATCTGGTGGCGGT
TTAGTTCAAGCCGGTGATAGCCTTCGTCTGAGCTGCGCCGCAAGCGGAGA
TTGTCACGTTCTCTGGCGTATGGATTGGTTTCGCCAGGCACCTGGTAAAG
AACGTGAATTTGTGGCAGCCATTAGCCTCGACGGTTTGTTTACCAACTAT
GCCGATTCTGTTCGTGGTCGCTTCAGCATTTCAGCAGATTCTGCGAAGAA
TACCGTGTACTTACAGATGAACTCACTGAAACCTGAGGATACCGCCGTTT
ATTATTGTGCGGCATTGCCGGCGACTTCTGGCGACTATTGGGGTCAGGGC
ACACAAGTCACGGTCTCAAGCGGCGGAAGCTCTGGTGAACAGAAACTGAT
TTCAGAAG
DCHVLWR#LDGLF#MPFF#evqlvesggglvqagds
lrlscaasgDCHVLWRmgwfrqapgkerefvaaisLDGLFtnyadsvrgr
fsisadsakntvylqmnslkpedtavyycvaMPFFdywgqgtqvtvss#A
TATACCATGGAAGTTCAACTGGTTGAATCTGGTGGCGGTTTAGTTCAAGC
CGGTGATAGCCTTCGTCTGAGCTGCGCCGCAAGCGGAGATTGTCACGTTC
TCTGGCGTATGGGTTGGTTTCGCCAGGCACCTGGTAAAGAACGTGAATTT
GTGGCAGCCATTAGCCTCGACGGTTTGTTTACCAACTATGCCGATTCTGT
TCGTGGTCGCTTCAGCATTTCAGCAGATTCTGCGAAGAATACCGTGTACT
TACAGATGAACTCACTGAAACCTGAGGATACCGCCGTTTATTATTGTGTG
GCAATGCCTTTTTTTGACTATTGGGGTCAGGGCACACAAGTCACGGTCTC
AAGCGGCGGAAGCTCTGGTGAACAGAAACTGATTTCAGAAG
DCH
VLWR#LDGLF#MPFF#evqlvesggglvqagdslrlscaasgDCHVLWRm
gwfrqapgkerefvaaisLDGLFtnyadsvrgrfsisadsakntvylqmn
slkpedtavyycaaMPFFdywgqgtqvtgss#GGAGATATACCATGGAAG
TTCAACTGGTTGAATCTGGTGGCGGTTTAGTTCAAGCCGGTGATAGCCTT
CGTCTGAGCTGCGCCGCAAGCGGAGATTGTCACGTTCTCTGGCGTATGGG
TTGGTTTCGCCAGGCACCTGGTAAAGAACGTGAATTTGTGGCAGCCATTA
GCCTCGACGGTTTGTTTACCAACTATGCCGATTCTGTTCGTGGTCGCTTC
AGCATTTCAGCAGATTCTGCGAAGAATACCGTGTACTTACAGATGAACTC
ACTGAAACCTGAGGATACCGCCGTTTATTATTGTGCGGCAATGCCTTTTT
TTGACTATTGGGGTCAGGGCACACAAGTCACGGGCTCAAGCGGCGGAAGC
TCTGGTGAACAGAAACTGATTTCAGAAG
DGHVLWR#LDGLF#MP
FF#evqlvesggglvqagdslrlscaasgDGHVLWRmgwfrqapgkeref
vaaisLDGLFtnyadsvrgrfsisadsakntvylqmnslkpedtavyyca
aMPFFdywgqgtqvtvss#AAGGAGATATACCATGGAAGTTCAACTGGTT
GAATCTGGTGGCGGTTTAGTTCAAGCCGGTGATAGCCTTCGTCTGAGCTG
CGCCGCAAGCGGAGATGGTCACGTTCTCTGGCGTATGGGTTGGTTTCGCC
AGGCACCTGGTAAAGAACGTGAATTTGTGGCAGCCATTAGCCTCGACGGT
TTGTTTACCAACTATGCCGATTCTGTTCGTGGTCGCTTCAGCATTTCAGC
AGATTCTGCGAAGAATACCGTGTACTTACAGATGAACTCACTGAAACCTG
AGGATACCGCCGTTTATTATTGTGCGGCAATGCCTTTTTTTGACTATTGG
GGTCAGGGCACACAAGTCACGGTCTCAAGCGGCGGAAGCTCTGGTGAACA
GAAACTGATTTCAGAAG
DRHVLWR#LDGLF#LPSVCT#evqlve
sggglvqagdslrlscatsgDRHVLWRmgwfrqapgkerefvaaisLDGL
FtnyadsvrgrfsisadsakntvylqmnslkpedtavyycaaLPSVCTdy
wgqgtqvtvss#GGAGATATACCATGGAAGTTCAACTGGTTGAATCTGGT
GGCGGTTTAGTTCAAGCCGGTGATAGCCTTCGTCTGAGCTGCGCCACAAG
CGGAGATCGTCACGTTCTCTGGCGTATGGGTTGGTTTCGCCAGGCACCTG
GTAAAGAACGTGAATTTGTGGCAGCCATTAGCCTCGACGGTTTGTTTACC
AACTATGCCGATTCTGTTCGTGGTCGCTTCAGCATTTCAGCAGATTCTGC
GAAGAATACCGTGTACTTACAGATGAACTCACTGAAACCTGAGGATACCG
CCGTTTATTATTGTGCGGCACTGCCTAGTGTGTGTACCGACTATTGGGGT
CAGGGCACACAAGTCACGGTCTCAAGCGGCGGAAGCTCTGGTGAACAGAA
ACTGATTTCAGAAG
DCHVLWR#LDGLF#HHFHPGGMD#evqlve
sggglvqagdslrlscaasgDCHVLWRmgwfrqapgkerefvaaisLDGL
FtnyadsvrgrfsisadsakntmylqmnslkpedtavyycnaHHFHPGGM
Ddywgqgtqvtvss#ATATACCATGGAAGTTCAACTGGTTGAATCTGGTG
GCGGTTTAGTTCAAGCCGGTGATAGCCTTCGTCTGAGCTGCGCCGCAAGC
GGAGATTGTCACGTTCTCTGGCGTATGGGTTGGTTTCGCCAGGCACCTGG
TAAAGAACGTGAATTTGTGGCAGCCATTAGCCTCGACGGTTTGTTTACCA
ACTATGCCGATTCTGTTCGTGGTCGCTTCAGCATTTCAGCAGATTCTGCG
AAGAATACCATGTACTTACAGATGAACTCACTGAAACCTGAGGATAGTGA
ACAGAAACTGATTTCAGAAG
DCHVLWR#LDGLF#LPATSG#evq
lvesggglvqagdslrlscaasgDCHVLWRmgwfrqapgkerefvaaisL
DGLFtnyadsvrgrfsisadsakntvylqmnslkpedtavyycaaLPATS
Gdywvqgtqvtvss#AGGAGATATACCATGGAAGTTCAACTGGTTGAATC
TGGTGGCGGTTTAGTTCAAGCCGGTGATAGCCTTCGTCTGAGCTGCGCCG
CAAGCGGAGATTGTCACGTTCTCTGGCGTATGGGTTGGTTTCGCCAGGCA
CCTGGTAAAGAACGTGAATTTGTGGCAGCCATTAGCCTCGACGGTTTGTT
TACCAACTATGCCGATTCTGTTCGTGGTCGCTTCAGCATTTCAGCAGATT
CTGCGAAGAATACCGTGTACTTACAGATGAACTCACTGAAACCTGAGGAT
ACCGCCGTTTATTATTGTGCGGCATTGCCGGCGACTTCTGGCGACTATTG
GGTTCAGGGCACACAAGTCACGGTCTCAAGCGGCGGAAGCTCTGGTGAAC
AGAAACTGATTTCAGAAG
DCHVLWS#LDGLF#MPFF#evqlves
ggglvqagdslrlscaasgDCHVLWSmgwfrqapgkerefvaaisLDGLF
tnyadsvrgrfsisadsakntvylqmnslkpedtavyycaaMPFFdywgq
gtqvtvss#GGAGATATACCATGGAAGTTCAACTGGTTGAATCTGGTGGC
GGTTTAGTTCAAGCCGGTGATAGCCTTCGTCTGAGCTGCGCCGCAAGCGG
AGATTGTCACGTTCTCTGGAGTATGGGTTGGTTTCGCCAGGCACCTGGTA
AAGAACGTGAATTTGTGGCAGCCATTAGCCTCGACGGTTTGTTTACCAAC
TATGCCGATTCTGTTCGTGGTCGCTTCAGCATTTCAGCAGATTCTGCGAA
GAATACCGTGTACTTACAGATGAACTCACTGAAACCTGAGGATACCGCCG
TTTATTATTGTGCGGCAATGCCTTTTTTTGACTATTGGGGTCAGGGCACA
CAAGTCACGGTCTCAAGCGGCGGAAGCTCTGGTGAACAGAAACTGATTTC
AGAAG
DCHVLWR#LDGLF#MPFF#evqlvesggglvqagdslrl
scaasgDCHVLWRmgwfrqapgkerefvaaisLDGLFtnyadsvrgrfsi
sadsakntvylqmnslkpedtavyycaaMPFFdywgqgtqvtvsr#GAGA
TATACCATGGAAGTTCAACTGGTTGAATCTGGTGGCGGTTTAGTTCAAGC
CGGTGATAGCCTTCGTCTGAGCTGCGCCGCAAGCGGAGATTGTCACGTTC
TCTGGCGTATGGGTTGGTTTCGCCAGGCACCTGGTAAAGAACGTGAATTT
GTGGCAGCCATTAGCCTCGACGGTTTGTTTACCAACTATGCCGATTCTGT
TCGTGGTCGCTTCAGCATTTCAGCAGATTCTGCGAAGAATACCGTGTACT
TACAGATGAACTCACTGAAACCTGAGGATACCGCCGTTTATTATTGCGCG
GCAATGCCTTTTTTTGACTATTGGGGTCAGGGCACACAAGTCACGGTCTC
ACGCGGCGGAAGCTCTGGTGAACAGAAACTGATTTCAGAAG
DCH
VLWR#LDGLF#MPFF#evqlvesggglvqagdslrlscaasgDCHVLWRm
gwfrqapgkerefvaaisLDGLFtnyadsvrgrfsisadsakntvylqmn
slkpedtavyycaaMPFFdywgqgtqvtfss#ACCATGGAAGTTCAACTG
GTTGAATCTGGTGGCGGTTTAGTTCAAGCCGGTGATAGCCTTCGTCTGAG
CTGCGCCGCAAGCGGAGATTGTCACGTTCTCTGGCGTATGGGTTGGTTTC
GCCAGGCACCTGGTAAAGAACGTGAATTTGTGGCAGCCATTAGCCTCGAC
GGTTTGTTTACCAACTATGCCGATTCTGTTCGTGGTCGCTTCAGCATTTC
AGCAGATTCTGCGAAGAATACCGTGTACTTACAGATGAACTCACTAAAAC
CTGAGGATACCGCCGTTTATTATTGTGCGGCAATGCCTTTTTTTGACTAT
TGGGGTCAGGGCACACAAGTCACGTTCTCAAGCGGCGGAAGCTCTGATGA
ACAGAAACTGATTTCAGAAG
DCHVLWR#LDGLF#MPFF#evqlv
esggglvqagdslrlscaasgDCHVLWRmgwfrqapgkerefvaaisLDG
LFtnyadsvrgrfsisadsakntvylqmnslkpedtavyycaaMPFFdhw
gqgtqvtvss#AAGGAGATATACCATGGAAGTTCAACTGGTTGAATCTGG
TGGCGGTTTAGTTCAAGCCGGTGATAGCCTTCGTCTGAGCTGCGCCGCAA
GCGGAGATTGTCACGTTCTCTGGCGTATGGGTTGGTTTCGCCAGGCACCT
GGTAAAGAACGTGAATTTGTGGCAGCCATTAGCCTCGACGGTTTGTTTAC
CAACTATGCCGATTCTGTTCGTGGTCGCTTCAGCATTTCAGCAGATTCTG
CGAAGAATACCGTGTACTTACAGATGAACTCACTGAAACCTGAGGATACC
GCCGTTTATTATTGTGCGGCAATGCCTTTTTTTGACCATTGGGGTCAGGG
CACACAAGTCACGGTCTCAAGCGGCGGAAGCTCTGGTGAACAGAAACTGA
TTTCAGAAG
DCHVLWR#LDGLF#HHFHPGGMD#evqlvesgggl
vqaggsIrlscaasgDCHVLWRmgwfrqapgkerefvaaisLDGLFtnya
dsvrgrfsisadsakntvylqmnslkpedtavyycnaHHFHPGGMDdywg
qgtqvtvss#AAGGAGATATACCATGGAAGTTCAACTGGTTGAATCTGGT
GGCGGTTTAGTTCAAGCCGGTGGTAGCCTTCGTCTGAGCTGCGCCGCAAG
CGGAGATTGTCACGTTCTCTGGCGTATGGGTTGGTTTCGCCAGGCACCTG
GTAAAGAACGTGAATTTGTGGCAGCCATTAGCCTCGACGGTTTGTTTACC
AACTATGCCGATTCTGTTCGTGGTCGCTTCAGCATTTCAGCAGATTCTGC
GAAGAATACCGTGTACTTACAGATGAACTCACTGAAACCTGGCTCTGGTG
AACAGAAACTGATTTCAGAAG
DCHVLWR#LDGLF#MPFF#evql
vesggglvqagdslrlscaasgDCHVLWRmgwfrqapgkerefvaaisLD
GLFtnyadsvrgrfsisadsakntvylqmnslkpedtavyycaaMPFFay
wvqgtqvtvss#AGATATACCATGGAAGTTCAACTGGTTGAATCTGGTGG
CGGTTTAGTTCAAGCCGGTGATAGCCTTCGTCTGAGCTGCGCCGCAAGCG
GAGATTGTCACGTTCTCTGGCGTATGGGTTGGTTTCGCCAGGCACCTGGT
AAAGAACGTGAATTTGTGGCAGCCATTAGCCTCGACGGTTTGTTTACCAA
CTATGCCGATTCTGTTCGTGGTCGCTTCAGCATTTCAGCAGATTCTGCGA
AGAATACCGTGTACTTACAGATGAACTCACTGAAACCTGAGGATACCGCC
GTTTATTATTGTGCGGCAATGCCTTTTTTTGCCTATTGGGTTCAGGGCAC
ACAAGTCACGGTCTCAAGCGGCGGAAGCTCTGGTGAACAGAAACTGATTT
CAGAAG
DCHVLWR#LDGLF#LPKRTV#qvqlvesggalvqagds
lrlscaasgDCHVLWRmgwfrqtpgkerefvaaisLDGLFtnyadsvrgr
fsisadsakntvylqmnslkpedtavyycaaLPGATTGTCACGTTCTCTG
GCGTATGGGTTGGTTTCGCCAGACACCTGGTAAAGAACGTGAATTTGTGG
CAGCCATTAGCCTCGACGGTTTGTTTACCAACTATGCCGATTCTGTTCGT
GGTCGCTTCAGCATTTCAGCAGATTCTGCGAAGAATACCGTGTACTTACA
GATGAACTCACTGAAACCTGAGGATACCGCCGTTTATTATTGTGCGGCAC
TGCCTAAGCGGACTGTGGACTATTGGGGTCAGGGCACACAAGTCACGGTC
TCAAGCGGCGGAAGCTCTGGTGAACAGAAACTGATTTCAGAAG
D
CHVLWR#LDGLF#MPFF#evqlvesggglvqagdslrlscaasgDCHVLW
RmgwfrqapgkerefvaaisLDGLFtnyadsvrgrfsisadpakntvylq
mnslkpedtavyycaaMPFFdywgqgtqvtvss#ACCATGGAAGTTCAAC
TGGTTGAATCTGGTGGCGGTTTAGTTCAAGCCGGTGATAGCCTTCGTCTG
AGCTGCGCCGCAAGCGGAGATTGTCACGTTCTCTGGCGTATGGGTTGGTT
TCGACAGGCACCTGGTAAAGAACGTGAATTTGTGGCAGCCATTAGCCTCG
ACGGTTTGTTTACCAACTATGCCGATTCTGTTCGTGGTCGCTTCAGCATT
TCAGCAGATCCTGCGAAGAATACCGTGTACTTACAGATGAACTCACTGAA
ACCTGAGGATACCGCCGTTTATTATTGTGCGGCAATGCCTTTTTTTGACT
ATTGGGGTCAGGGCACACAAGTCACGGTCTCAAGCGGCGGAAGCTCTGGT
GAACAGAAACTGATTTCAGAAG
DCHVLWR#LDGLF#LPATSG#e
vqlvesggglvqvgdslrlscaasgDCHVLWRmgwfrqapgkerefvaai
sLDGLFtnyadsvrgrfsisadsakntvylqmnslkpedtavyycaaLPA
TSGdywgqgtqvtvss#GAAGGAGATATACCATGGAAGTTCAACTGGTTG
AATCTGGTGGCGGTTTAGTTCAAGTCGGTGATAGCCTTCGTCTGAGCTGC
GCCGCAAGCGGAGATTGTCACGTTCTCTGGCGTATGGGTTGGTTTCGCCA
GGCACCTGGTAAAGAACGTGAATTTGTGGCAGCCATTAGCCTCGACGGTT
TGTTTACCAACTATGCCGATTCTGTTCGTGGTCGCTTCAGCATTTCAGCA
GATTCTGCGAAGAATACCGTGTACTTACAGATGAACTCACTGAAACCTGA
GGATACCGCCGTTTATTATTGTGCGGCATTGCCGGCGACTTCTGGCGACT
ATTGGGGTCAGGGCACACAAGTCACGGTCTCAAGCGGCGGAAGCTCTGGT
GAACAGAAACTGATTTCAGAAG
DCHVLWR#LDGLF#MPFF#evq
lvesggglvqagdslrlscaasgDCHVLWRmgwfrqapgkerefvaaisL
DGLFtnyadsvcgrfsisadsakntvylqmnslkpedtavyycaaMPFFd
ywgqgtqvtvss#AAGGAGATATACCATGGAAGTTCAACTGGTTGAATCT
GGTGGCGGTTTAGTTCAAGCCGGTGATAGCCTTCGTCTGAGCTGCGCCGC
AAGCGGAGATTGTCACGTTCTCTGGCGTATGGGTTGGTTTCGCCAGGCAC
CTGGTAAAGAACGTGAATTTGTGGCAGCCATTAGCCTCGACGGTTTGTTT
ACCAACTATGCCGATTCTGTTTGTGGTCGCTTCAGCATTTCAGCAGATTC
TGCGAAGAATACCGTGTACTTACAGATGAACTCACTGAAACCTGAGGATA
CCGCCGTTTATTATTGTGCGGCAATGCCTTTTTTTGACTATTGGGGTCAG
GGCACACAAGTCACGGTCTCAAGCGGCGGAAGCTCTGGTGAACAGAAACT
GATTTCAGAAG
DCHVLWR#LDGLF#MPFF#evqlvesggglvqa
gdslrlscaasgDCHVLWRmgwfrqapgkerefvaaisLDGLFtnyadsv
rgrfsisadsakntvylqmnslkpedtavyycaaMPFFdywgrgtqvtvs
s#AAGGAGATATACCATGGAAGTTCAACTGGTTGAATCTGGTGGCGGTTT
AGTTCAAGCCGGTGATAGCCTTCGTCTGAGCTGCGCCGCAAGCGGAGATT
GTCACGTTCTCTGGCGTATGGGTTGGTTTCGCCAGGCACCTGGTAAAGAA
CGTGAATTTGTGGCAGCCATTAGCCTCGACGGTTTGTTTACCAACTATGC
CGATTCTGTTCGTGGTCGCTTCAGCATTTCAGCAGATTCTGCGAAGAATA
CCGTGTACTTACAGATGAACTCACTGAAACCTGAGGATACCGCCGTTTAT
TATTGTGCGGCAATGCCTTTTTTTGACTATTGGGGTCGGGGCACACAAGT
CACGGTCTCAAGCGGCGGAAGCTCTGGTGAACAGAAACTGATTTCAGAAG
DCHVLWR#LDGLF#CTRFRA#evqlvesggglvqagdslrlsca
asgDCHVLWRmgwfrqapgkerefvaaisLDGLFtnyadsvrgrfsisad
sakntvylqmnslkpedtavyycaaCTRFRAdywgqgtqvtvss#AAGGA
GATATACCATGGAAGTTCAACTGGTTGAATCTGGTGGCGGTTTAGTTCAA
GCCGGCGATAGCCTTCGTCTGAGCTGCGCCGCAAGCGGAGATTGTCACGT
TCTCTGGCGTATGGGTTGGTTTCGCCAGGCACCTGGTAAAGAACGTGAAT
TTGTGGCAGCCATTAGCCTCGACGGTTTGTTTACCAACTATGCCGATTCT
GTTCGTGGTCGCTTCAGCATTTCAGCAGATTCTGCGAAGAATACCGTGTA
CTTACAGATGAACTCACTGAAACCTGAGGATACCGCCGTTTATTATTGTG
CGGCATGTACCAGGTTCAGGGCCGACTATTGGGGTCAGGGCACACAAGTC
ACGGTCTCAAGCGGCGGAAGCTCTGGTGAACAGAAACTGATTTCAGAAG
DCHVLWR#LDGLF#LPATSG#evqlvesggglvkagdslrlscaa
sgDCHVLWRmgwfrqapgkerefvaaisLDGLFtnyadsvrgrfsisads
akntvylqmnslkpedtavyycaaLPATSGaywgqgtqvtvss#GGAGAT
ATACCATGGAAGTTCAACTGGTTGAATCTGGTGGCGGTTTAGTTAAAGCC
GGTGATAGCCTTCGTCTGAGCTGCGCCGCAAGCGGAGATTGTCACGTTCT
CTGGCGTATGGGTTGGTTTCGCCAGGCACCTGGTAAAGAACGTGAATTTG
TGGCAGCCATTAGCCTCGACGGTTTGTTTACCAACTATGCCGATTCTGTT
CGTGGTCGCTTCAGCATTTCAGCAGATTCTGCGAAGAATACCGTGTACTT
ACAGATGAACTCACTGAAACCTGAGGATACCGCCGTTTATTATTGTGCGG
CATTGCCGGCGACTTCTGGCGCCTATTGGGGTCAGGGCACACAAGTCACG
GTCTCAAGCGGCGGAAGCTCTGGTGAACAGAAACTGATTTCAGAAG
DCHVLWR#LDGLF#MPFF#evqlvesggglvqagdrlrlscaasgDCH
VLWRmgwfrqapgkerefvaaisLDGLFtnyadsvrgrfsisadsakntv
ylqmnslkpedtavyycaaMPFFdywgqgtqvtvss#GAAGGAGATATAC
CATGGAAGTTCAACTGGTTGAATCTGGTGGCGGTTTAGTTCAAGCCGGTG
ATCGCCTTCGTCTGAGCTGCGCCGCAAGCGGAGATTGTCACGTTCTCTGG
CGTATGGGTTGGTTTCGCCAGGCACCTGGTAAAGAACGTGAATTTGTGGC
AGCCATTAGCCTCGACGGTTTGTTTACCAACTATGCCGATTCTGTTCGTG
GTCGCTTCAGCATTTCAGCAGATTCTGCGAAGAATACCGTGTACTTACAG
ATGAACTCACTGAAACCTGAGGATACCGCCGTTTATTATTGTGCGGCAAT
GCCTTTTTTTGACTATTGGGGTCAGGGCACACAAGTCACGGTCTCAAGCG
GCGGAAGCTCTGGTGAAAAGAAACTGATTTCAGAAGCCAA
DCHV
LWR#LDGLF#MPFF#evqlvesggglvqagdslrlscaasgDCHVLWRmg
wfrqapgkerefaaaisLDGLFtnyadsvrgrfsisadsakntvylqmns
lkpedtavyycaaMPFFdywgqgtqvtvss#ATACCATGGAAGTTCAACT
GGTTGAATCTGGTGGCGGTTTAGTTCAAGCCGGTGATAGCCTTCGTCTGA
GCTGCGCCGCAAGCGGAGATTGTCACGTTCTCTGGCGTATGGGTTGGTTT
CGCCAGGCACCTGGTAAAGAACGTGAATTTGCGGCAGCCATTAGCCTCGA
CGGTTTGTTTACCAACTATGCCGATTCTGTTCGTGGTCGCTTCAGCATTT
CAGCAGATTCTGCGAAGAATACCGTGTACTTACAGATGAACTCACTGAAA
CCTGAGGATACCGCCGTTTATTATTGTGCGGCAATGCCTTTTTTTGACTA
TTGGGGTCAGGGCACACAAGTCACGGTCTCAAGCGGCGGAAGCTCTGGTG
AACAGAAACTGATTTCAGAAGCCAA
DCHVLWR#LDGLF#HNFHP
GGMD#evqlvesggglvqagdslrlscaasgDCHVLWRmgwfrqapgker
efvaaisLDGLFtnyadsvrgrfsisadsakntvylqmnslkpedtavyy
cnaHNFHPGGMDdywgqgtqvtvss#AGATATACCATGGAAGTTCAACTG
GTTGAATCTGGTGGCGGTTTAGTTCAAGCCGGTGATAGCCTTCGTCTGAG
CTGCGCCGCAAGCGGAGATTGTCACGTTCTCTGGCGTATGGGTTGGTTTC
GCCAGGCACCTGGTAAAGAACGTGAATTTGTGGCAGCCATTAGCCTCGAC
GGTTTGTTTACCAACTATGCCGATTCTGTTCGTGGTCGCTTCAGCATTTC
AGCAGATTCTGCGAAGAATACCGTGTACTTACAGATGAACTCACTGAAAC
CTGAGGATACCGCCGTTTATTATTGTAATGCACACAATTTTCATCCTGGT
GGTATGGATGACTATTGGGGTCAGGGCACACAAGTCACGGTCTCAAGCGG
CGGAAGCTCTGGTGAACAGAAACTGATTTCAGAAGCCAA
DCHVL
SR#LDGLF#TIYGVY#evqlvesggglvqagdslrlscaasgDCHVLSRm
gwfrqapgkerefvaaisLDGLFtnyadsvrgrfgisadsakntvylqmn
slkpedtavyycaaTIYGVYeywgqgtqvtvss#AGGAGATATACCATGG
AAGTTCAACTGGTTGAATCTGGTGGCGGTTTAGTTCAAGCCGGTGATAGC
CTTCGTCTGAGCTGCGCCGCAAGCGGAGATTGTCACGTTCTCTCGCGTAT
GGGTTGGTTTCGCCAGGCACCTGGTAAAGAACGTGAATTTGTGGCAGCCA
TTAGCCTCGACGGTTTGTTTACCAACTATGCCGATTCTGTTCGTGGTCGC
TTCGGCATTTCAGCAGATTCTGCGAAGAATACCGTGTACTTACAGATGAA
CTCACTGAAACCTGAGGATACCGCCGTTTATTATTGTGCGGCAACTATCT
ATGGTGTCTATGAGTATTGGGGTCAGGGCACACAAGTCACGGTCTCAAGC
GGCGGAAGCTCTGGTGAACAGAAACTGATTTCAGAAGCCAA
DCH
VLWR#LDGLF#MPFF#evqlvesggglvqagdslrlscaasgDCHVLWRm
gwfrqapgkerefvaaisLDGLFtnyadsvrgrfsisadsakntvylqmn
slkpedtavyycaaMPFFdywgqgtqgtvss#ACCATGGAAGTTCAACTG
GTTGAATCTGGTGGCGGTTTAGTTCAAGCCGGTGATAGCCTTCGTCTGAG
CTGCGCCGCAAGCGGAGATTGTCACGTTCTCTGGCGTATGGGTTGGTTTC
GCCAGGCACCTGGTAAAGAACGTGAATTTGTGGCAGCCATTAGCCTCGAC
GGTTTGTTTACCAACTATGCCGATTCTGTTCGTGGTCGCTTCAGCATTTC
AGCAGATTCTGCGAAGAATACCGTGTACTTACAGATGAACTCACTGAAAC
CTGAGGATACCGCCGTTTATTATTGTGCGGCAATGCCTTTTTTTGACTAT
TGGGGTCAGGGCACACAAGGCACGGTCTCAAGCGGCGGAAGCTCTGGTGA
ACAGAAACTGATTTCAGAAGCCAA
DCHVLWR#LDGLF#LPATSGevqlvesggglvqagdslrlscaasgDCHV
LWRmgwfrqapgkerefva
aisLDGLFtnyadsvrgrfsisadsakntvylqmnslkpedtavyycaaL
PATSGdywgqgtqgtvss#GGAGATATACCATGGAAGTTCAACTGGTTGA
ATCTGGTGGCGGTTTAGTTCAAGCCGGTGATAGCCTTCGTCTGAGCTGCG
CCGCAAGCGGAGATTGTCACGTTCTCTGGCGTATGGGTTGGTTTCGCCAG
GCACCTGGTAAAGAACGTGAATTTGTGGCAGCCATTAGCCTCGACGGTTT
GTTTACCAACTATGCCGATTCTGTTCGTGGTCGCTTCAGCATTTCAGCAG
ATTCTGCGAAGAATACCGTGTACTTACAGATGAACTCACTGAAACCTGAG
GATACCGCCGTTTATTATTGTGCGGCATTGCCGGCGACTTCTGGCGACTA
TTGGGGTCAGGGCACACAAGGTACGGTCTCAAGCGGCGGAAGCTCTGGTG
AACAGAAACTGATTTCAGAAGCCAA
DCHVLWR#LDGLF#MPFF#
evqlvesggglvhagdslrlscaasgDCHVLWRmgwfrqapgkerefvaa
isLDGLFtnyadsvrgrfsisadsakntvylqmnslkpedtavyycaaMP
FFdywaqgthvtvss#GAAGGAGATATACCATGGAAGTTCAACTGGTTGA
ATCTGGTGGCGGTTTAGTTCACGCCGGTGATAGCCTTCGTCTGAGCTGCG
CCGCAAGCGGAGATTGTCACGTTCTCTGGCGTATGGGTTGGTTTCGCCAG
GCACCTGGTAAAGAACGTGAATTTGTGGCAGCCATTAGCCTCGACGGTTT
GTTTACCAACTATGCCGATTCTGTTCGTGGTCGCTTCAGCATTTCAGCAG
ATTCTGCGAAGAATACCGTGTACTTACAGATGAACTCACTGAAACCTGAG
GATACCGCCGTTTATTATTGTGCGGCAATGCCTTTTTTTGACTATTGGGC
TCAGGGCACACATGTCACGGTCTCAAGCGGCGGAAGCTCTGGTGCACAGA
AACTGATCTCAGAAGCCAA
DCHVLWR#LDGLF#MPFF#qvqlqe
sggglvqagdslrlscaasgDCHVLWRmgwfrqapgkerefvaaisLDGL
FtnyadsvrgrfsisadsakntvylqmnslkpedtavyycaaMPFFdywg
qgtqvtvss#ATATACCATGCAGGTTCAACTGCAAGAATCTGGTGGCGGT
TTAGTTCAAGCCGGTGATAGCCTTCGTCTGAGCTGCGCCGCAAGCGGAGA
TTGTCACGTTCTCTGGCGTATGGGTTGGTTTCGCCAGGCACCTGGTAAAG
AACGTGAATTTGTGGCAGCCATTAGCCTCGACGGTTTGTTTACCAACTAT
GCCGATTCTGTTCGTGGTCGCTTCAGCATTTCAGCAGATTCTGCGAAGAA
TACCGTGTACTTACAGATGAACTCACTGAAACCTGAGGATACCGCCGTTT
ATTATTGTGCGGCAATGCCTTTTTTTGACTATTGGGGTCAGGGCACACAA
GTCACGGTCTCAAGCGGCGGAAGCTCTGGTGAACAGAAACTGATTTCAGA
AGCCAA
DCHVLWR#LDGLF#LPATSG#evqlvesggglvqagds
lrlscaasgDCHVLWRmgwfrqapgkereivaaisLDGLFtnyadsvrgr
fsisadsakntvylqmnslkpedtavyycaaLPATSGdcwgqgtqvtvss
ACCATGGAAGTTCAACTGGTTGAATCTGGTGGCGGTTTAGTTCAAGCCG
GTGATAGCCTTCGTCTGAGCTGCGCCGCAAGCGGAGATTGTCACGTTCTC
TGGCGTATGGGTTGGTTTCGCCAGGCACCTGGTAAAGAACGTGAAATTGT
GGCAGCCATTAGCCTCGACGGTTTGTTTACCAACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGGC
ATTGCCGGCGACTTCTGGCGACTGTTGGGGTCAGGGCACACAAGTCACGG
TCTCAAGCGGCGGAAGCTCTGGTGAACAGAAACTGATTTCAGAAGCCAA
DCHVLWR#LDGLF#LTSPLSDSS#evqlfesggglvqagdslrls
caasgDCHVLWRmgwfrqapgkerefvaaisLDGLFtnyadsvrgrfsis
adsakntvylqmnslkpedtavyycaaLTSPLSDSSdywgqgtqvtvss#
ATATACCATGGAAGTTCAACTGTTTGAATCTGGTGGCGGTTTAGTTCAAG
CCGGTGATAGCCTTCGTCTGAGCTGCGCCGCAAGCGGAGATTGTCACGTT
CTCTGGCGTATGGGTTGGTTTCGCCAGGCACCTGGTAAAGAACGTGAATT
TGTGGCAGCCATTAGCCTCGACGGTTTGTTTACCAACTATGCCGATTCTG
TTCGTGGTCGCTTCAGCATTTCAGCAGATTCTGCGAAGAATACCGTGTAC
TTACAGATGAACTCACTGAAACCTGAGGACACCGCCGTTTATTATTGTGC
GGCATTGACGTCTCCGCTTTCGGATTCGTCCGACTATTGGGGTCAGGGCA
CACAAGTCACGGTCTCAAGCGGCGGAAGCTCTGGTGAACAGAAACTGATT
TCAGAAGCCAA
DCHVLWR#LDGLF#MPFF#evqlvesggglvqa
gdslrlscaasgDCHVLWRmgwfrqapgkerefvaaisLDGLFtnyadsv
rgrfsisadsakntvylqmnslkpedtavyycaaMPFFdywgqgtqvtvs
i#ATATACCATGGAAGTTCAACTGGTTGAATCTGGTGGCGGTTTAGTTCA
AGCCGGTGATAGCCTTCGTCTGAGCTGCGCCGCAAGCGGAGATTGTCACG
TTCTCTGGCGTATGGGTTGGTTTCGCCAGGCACCTGGTAAAGAACGTGAA
TTTGTGGCAGCCATTAGCCTCGACGGTTTGTTTACCAACTATGCCGATTC
TGTTCGTGGTCGCTTCAGCATTTCAGCAGATTCTGCGAAGAATACCGTGT
ACTTACAGATGAACTCACTGAAACCTGAGGATACCGCCGTTTATTATTGT
GCGGCAATGCCTTTTTTTGACTATTGGGGTCAGGGCACACAAGTCACGGT
CTCAATCGGCGGAAGCTCTGGTGAACAGAAACTGATTTCAGAAGCCAA
DCHVLWR#LDGLF#MPFF#evqlvesggglvqtgdslrlscaasgD
CHVLWRmgwfrqapgkerefvaaisLDGLFtnyadsvrgrfsisadsakn
tvylqmnslkpedtavyycaaMPFFdywgqgtqvtvss#GGAGATATACC
ATGGAAGTTCAACTGGTTGAATCTGGTGGCGGTTTAGTTCAAACCGGTGA
TAGCCTTCGTCTGAGCTGCGCCGCAAGCGGAGATTGTCACGTTCTCTGGC
GTATGGGTTGGTTTCGCCAGGCACCTGGTAAAGAACGTGAATTTGTGGCA
GCCATTAGCCTCGACGGTTTGTTTACCAACTATGCCGATTCTGTTCGTGG
TCGCTTCAGCATTTCAGCAGATTCTGCGAAGAATACCGTGTACTTACAGA
TGAACTCACTGAAACCTGAGGATACCGCCGTTTATTATTGTGCGGCAATG
CCTTTTTTTGACTATTGGGGTCAGGGCACACAAGTCACGGTCTCAAGCGG
CGGAAGCTCTGGTGAACAGAAACTGATTTCAGAAGCCAA
DCHIL
WR#LDGLF#LPSVCT#evqlvesggglvqagdslrlscaasgDCHILWRm
gwfrqapgkerefvaaisLDGLFtnyadsvrgrfsisadsakntvylqmn
slkpedtavyycaaLPSVCTdywgqgtqgtvss#ATATACCATGGAAGTT
CAACTGGTTGAATCTGGTGGCGGTTTAGTTCAAGCCGGTGATAGCCTTCG
TCTGAGCTGCGCCGCAAGCGGAGATTGTCACATTCTCTGGCGTATGGGTT
GGTTTCGCCAGGCACCTGGTAAAGAACGTGAATTTGTGGCAGCCATTAGC
CTCGACGGTTTGTTTACCAACTATGCCGATTCTGTTCGTGGTCGCTTCAG
CATTTCAGCAGATTCTGCGAAGAATACCGTGTACTTACAGATGAACTCAC
TGAAACCTGAGGATACCGCCGTTTATTATTGTGCGGCACTGCCTAGTGTG
TGTACCGACTATTGGGGGCAGGGCACACAAGGCACGGTCTCAAGCGGCGG
AAGCTCTGGTGAACAGAAACTGATTTCAGAAGCCAA
DCHVLWR#
LDGLF#MPFF#evqlvesggglvqagdslrlscaasgDCHVLWRmgwfrq
apgkerefvaaisLDGLFtnyadsvrghfsisadsakntvylqmnslkpe
dtavyycaaMPFFdywgqgtqvtvss#AGGAGATATACCATGGAAGTTCA
ACTGGTTGAATCTGGTGGCGGTTTAGTTCAAGCCGGTGATAGCCTTCGTC
TGAGCTGCGCCGCAAGCGGAGATTGTCACGTTCTCTGGCGTATGGGTTGG
TTTCGCCAGGCACCTGGTAAAGAACGTGAATTTGTGGCAGCCATTAGCCT
CGACGGTTTGTTTACCAACTATGCCGATTCTGTTCGTGGTCACTTCAGCA
TTTCAGCAGATTCTGCGAAGAATACCGTGTACTTACAGATGAACTCACTG
AAACCTGAGGATACCGCCGTTTATTATTGTGCGGCAATGCCTTTTTTTGA
CTATTGGGGTCAGGGCACACAAGTCACGGTCTCAAGCGGCGGAAGCTCTG
GTGAACAGAAACTGATTTCAGAAGCCAA
DFHVLWR#LDGLF#LP
ATSG#evqlvesggglvqagdslrlscaasgDFHVLWRmgwfrqapgker
efvaaisLDGLFtnyadsvrgrfsisadsakntvylqmnslkpedtavyy
caaLPATSGdywgqgtqvtvss#GGAGATATACCATGGAAGTTCAACTGG
TTGAATCTGGTGGCGGTTTAGTTCAAGCCGGTGATAGCCTTCGTCTGAGC
TGCGCCGCAAGCGGAGATTTTCACGTTCTCTGGCGTATGGGTTGGTTTCG
CCAGGCACCTGGTAAAGAACGTGAATTTGTGGCAGCCATTAGCCTCGACG
GTTTGTTTACCAACTATGCCGATTCTGTTCGTGGTCGCTTCAGCATTTCA
GCAGATTCTGCGAAGAATACCGTGTACTTACAGATGAACTCACTGAAACC
TGAGGATACCGCCGTTTATTATTGTGCGGCATTGCCGGCGACTTCTGGCG
ACTATTGGGGTCAGGGCACACAAGTCACGGTCTCAAGCGGCGGAAGCTCT
GGTGAACAGAAACTGATTTCAGAAGCCAA
DCHVLWR#LDGLF#M
PFF#evqlvesggglvqagdslrlscaasgDCHVLWRmgwfrqapgkere
fvvaisLDGLFtnyadsvrgrfsisadsakntvylqmnslkpedtavyyc
aaMPFFdywgqgtqvtvss#GAAGGAGATATACCATGGAAGTTCAACTGG
TTGAATCTGGTGGCGGTTTAGTTCAAGCCGGTGATAGCCTTCGTCTGAGC
TGCGCCGCAAGCGGAGATTGTCACGTTCTCTGGCGTATGGGTTGGTTTCG
CCAGGCACCTGGTAAAGAACGTGAATTTGTGGTAGCCATTAGCCTCGACG
GTTTGTTTACCAACTATGCCGATTCTGTTCGTGGTCGCTTCAGCATTTCA
GCAGATTCTGCGAAGAATACCGTGTACTTACAGATGAACTCACTGAAACC
TGAGGATACCGCCGTTTATTATTGTGCGGCAATGCCTTTTTTTGACTATT
GGGGTCAGGGCACACAAGTCACGGTCTCAAGCGGCGGAAGCTCTGGTGAA
CAGAAACTGATTTCAGAAGCCAA
DCHVLWR#LDGLF#MPFF#ev
qlvesggglvqagdslrlscaasgDCHVLWRmgwfrqapgkerefvaais
LDGLFtnyadsvrgrfsisadsaknavylqmnslkpedtavyycaaMPFF
dywgqgtqvtvss#ACCATGGAAGTTCAACTGGTTGAATCTGGTGGCGGT
TTAGTTCAAGCCGGTGATAGCCTTCGTCTGAGCTGCGCCGCAAGCGGAGA
TTGTCACGTTCTCTGGCGTATGGGTTGGTTTCGCCAGGCACCTGGTAAAG
AACGTGAATTTGTGGCAGCCATTAGCCTCGACGGTTTGTTTACCAACTAT
GCCGATTCTGTTCGTGGTCGCTTCAGCATTTCAGCAGATTCTGCGAAGAA
TGCCGTGTACTTACAGATGAACTCACTGAAACCTGAGGATACCGCCGTTT
ATTATTGTGCGGCAATGCCTTTTTTTGACTATTGGGGTCAGGGCACACAA
GTCACGGTCTCAAGCGGCGGAAGCTCTGGTGAACAGAAACTGATTTCAGA
AGCCAA
DCHVLWR#LDGLF#LPATSG#evqlvesggglvqagds
lrlscaasgDCHVLWRmgwfrqapgkerefvaaisLDGLFtnyadsvrgr
fsisadsakntvylqmnllkpedtavyycaaLPATSGdywgqgtqvtvss
GGAGATATACCATGGAAGTTCAACTGGTTGAATCTGGTGGCGGTTTAGT
TCAAGCCGGTGATAGCCTTCGTCTGAGCTGCGCCGCAAGCGGAGATTGTC
ACGTTCTCTGGCGTATGGGTTGGTTTCGCCAGGCACCTGGTAAAGAACGT
GAATTTGTGGCAGCCATTAGCCTCGACGGTTTGTTTACCAACTATGCCGA
TTCTGTTCGTGGTCGCTTCAGCATTTCAGCAGATTCTGCGAAGAATACCG
TGTACTTACAGATGAACTTACTGAAACCTGAGGATACCGCCGTTTATTAT
TGTGCGGCATTGCCGGCGACTTCTGGCGACTATTGGGGTCAGGGCACACA
AGTCACGGTCTCAAGCGGCGGAAGCTCTGGTGAACAGAAACTGATTTCAG
AAGCCAA
DCHVLWR#LDGLF#MPFF#evqlvesggglvqdgdsv
rlscaasgDCHVLWRmgwfrqapgkerefvaaisLDGLFtnyadsvrgrf
sisadsakntvylqmnslkpedtavyycaaMPFFdywgqgtqvtvss#GA
AGGAGATATACCATGGAAGTTCAACTGGTTGAATCTGGTGGCGGTTTAGT
TCAAGACGGTGATAGCGTTCGTCTGAGCTGCGCCGCAAGCGGAGATTGTC
ACGTTCTCTGGCGTATGGGTTGGTTTCGCCAGGCACCTGGTAAAGAACGT
GAATTTGTGGCAGCCATTAGCCTCGACGGTTTGTTTACCAACTATGCCGA
TTCTGTTCGTGGTCGCTTCAGCATTTCAGCAGATTCTGCGAAGAATACCG
TGTACTTACAGATGAACTCACTGAAACCTGAGGATACCGCCGTTTATTAT
TGTGCGGCAATGCCTTTTTTTGACTATTGGGGTCAGGGCACACAAGTCAC
GGTCTCAAGCGGCGGAAGCTCTGGTGAACAGAAACTGATTTCAGAAGCCA
A
DCHVLWR#LVGLF#MPFF#evqlvesggglvqagdslrlscaa
sgDCHVLWRmgwfrqapgkerefvaaisLVGLFtnyvdsvrgrfsisads
akntvylqmnslkpedtavyycaaMPFFdywgqgtqvtvss#ATATACCA
TGGAAGTTCAACTGGTTGAATCTGGTGGCGGTTTAGTTCAAGCCGGTGAT
AGCCTTCGTCTGAGCTGCGCCGCAAGCGGAGATTGTCACGTTCTCTGGCG
TATGGGTTGGTTTCGCCAGGCACCTGGTAAAGAACGTGAATTTGTGGCAG
CCATTAGCCTCGTCGGTTTGTTTACCAACTATGTCGATTCTGTTCGTGGT
CGCTTCAGCATTTCAGCAGATTCTGCGAAGAATACCGTGTACTTACAGAT
GAACTCACTGAAACCTGAGGATACCGCCGTTTATTATTGTGCGGCAATGC
CTTTTTTTGACTATTGGGGTCAGGGCACACAAGTCACGGTCTCAAGCGGC
GGAAGCTCTGGTGAACAGAAACTGATTTCAGAAGCCAA
DCHVLW
R#LDGLF#HHFHPGGMD#evqlvesggglvqagdrlrlscaasgDCHVLW
RmgwfrqapgkerefvaaisLDGLFtnyadsvrgrfsisadsakntvylq
mnslkpedtavyycnaHHFHPGGMDdywgqgtqvtvss#GAAGGAGATAT
ACCATGGAAGTTCAACTGGTTGAATCTGGTGGCGGTTTAGTTCAAGCCGG
TGATCGCCTTCGTCTGAGCTGCGCCGCAAGCGGAGATTGTCACGTTCTCT
GGCGTATGGGTTGGTTTCGCCAGGCACCTGGTAAAGAACGTGAATTTGTG
GCAGCCATTAGCCTCGACGGTTTGTTTACCAACTATGCCGATTCTGTTCG
TGGTCGCTTCAGCATTTCAGCAGATTCTGCGAAGAATACCGTGTACTTAC
AGATGAACTCACTGAAACCTAGCTCTGGTGAACAGAAACTGATTTCAGAA
GCCAA
DCHVLWR#LDGLF#LPATSG#evqlvesggglvqagdsl
rlscaasgDCHVLWRmgwfrqapgkerefvaaisLDGLFtnyadsvrgrf
sisadsakntvylqmnslkpedtavyycaaLPATSGdywgqgtqvtfss#
AGGAGATATACCATGGAAGTTCAACTGGTTGAATCTGGTGGCGGTTTAGT
TCAAGCCGGTGATAGCCTTCGTCTGAGCTGCGCCGCAAGCGGAGATTGTC
ACGTTCTCTGGCGTATGGGTTGGTTTCGCCAGGCACCTGGTAAAGAACGT
GAATTTGTGGCAGCCATTAGCCTCGACGGTTTGTTTACCAACTATGCCGA
TTCTGTTCGTGGTCGCTTCAGCATTTCAGCAGATTCTGCGAAGAATACCG
TGTACTTACAGATGAACTCACTGAAACCTGAGGATACCGCCGTTTATTAT
TGTGCGGCATTGCCGGCGACTTCTGGCGACTATTGGGGTCAGGGCACACA
AGTCACGTTCTCAAGCGGCGGAAGCTCTGGTGAACAGAAACTGATTTCAG
AAGCCAA
DCHVLWR#ASYRY#WPILSA#evqlvesggglvqagd
slrlscaasgDCHVLWRmgwfrqapgkerefvaaitASYRYtdyadsvrg
rfsisadsakntvylqmnslkpedtavyycaaWPILSAdywgqgtqvtvs
s#GGAGATATACCATGGAAGTTCAACTGGTTGAATCTGGTGGCGGTTTAG
TTCAAGCCGGTGATAGCCTTCGTCTGAGCTGCGCCGCAAGCGGAGATTGT
CACGTTCTCTGGCGTATGGGTTGGTTTCGCCAGGCACCTGGTAAAGAACG
TGAATTTGTGGCAGCCATTACCGCGTCTTACCGTTACACCGACTATGCCG
ATTCTGTTCGTGGTCGCTTCAGCATTTCAGCAGATTCTGCGAAGAATACC
GTGTACTTACAGATGAACTCACTGAAACCTGAGGATACCGCCGTTTATTA
TTGTGCGGCATGGCCCATTCTGTCCGCTGACTATTGGGGTCAGGGCACAC
AAGTCACGGTCTCAAGCGGCGGAAGCTCTGGTGAACAGAAACTGATTTCA
GAAGCCAA
DCHVLWR#LDGLF#LLATSG#evqlvesggglvqag
dslrlscaasgDCHVLWRmgwfrqapgkerefvaaisLDGLFtnyadsvr
grfsisadsakntvylqmnslkpedtavyycaaLLATSGdywgqgtqvtv
ss#ATATACCATGGAAGTTCAACTGGTTGAATCTGGTGGCGGTTTAGTTC
AAGCCGGTGATAGCCTTCGTCTGAGCTGCGCCGCAAGCGGAGATTGTCAC
GTTCTCTGGCGTATGGGTTGGTTTCGCCAGGCACCTGGTAAAGAACGTGA
ATTTGTGGCAGCCATTAGCCTCGACGGTTTGTTTACCAACTATGCCGATT
CTGTTCGTGGTCGCTTCAGCATTTCAGCAGATTCTGCGAAGAATACCGTG
TACTTACAGATGAACTCACTGAAACCTGAGGATACCGCCGTTTATTATTG
TGCGGCATTGCTGGCGACTTCTGGCGACTATTGGGGTCAGGGCACACAAG
TCACGGTCTCAAGCGGCGGAAGCTCTGGTGAACAGAAACTGATTTCAGAA
GCCAA
DCHVLWR#LDGLF#MPFF#evqlvesggglvhagdslrl
scaasgDCHVLWRmgwfrqapgkerefvaaisLDGLFtnyadsvrgrfsi
sadsakntvylqmnslkpedtavyycaaMPFFdywgqgtqvtvss#GAAG
GAGATATACCATGGAAGTTCAACTGGTTGAATCTGGTGGCGGTTTAGTTC
ACGCCGGTGATAGCCTTCGTCTGAGCTGCGCCGCAAGCGGAGATTGTCAC
GTTCTCTGGCGTATGGGTTGGTTTCGCCAGGCACCTGGTAAAGAACGTGA
ATTTGTGGCAGCCATTAGCCTCGACGGTTTGTTTACCAACTATGCCGATT
CTGTTCGTGGTCGCTTCAGCATTTCAGCAGATTCTGCGAAGAATACCGTG
TACTTACAGATGAACTCACTGAAACCTGAGGATACCGCCGTTTATTATTG
TGCGGCAATGCCTTTTTTTGACTATTGGGGTCAGGGCACACAAGTCACGG
TCTCAAGCGGCGGAAGCTCTGGTGAACAGAAACTGATTTCAGAAGCCAA
DCHVLWR#LDGLF#MPFF#evqlvesggglvqagdslrlrcaasg
DCHVLWRmgwfrqapgkerefvaaisLDGLFtnyadsvrgrfsisadsak
ntvylqmnslkpedtavyycaaMPFFdywgqgtqvtvss#AGGAGATATA
CCATGGAAGTTCAACTGGTTGAATCTGGTGGCGGTTTAGTTCAAGCCGGT
GATAGCCTTCGTCTGCGCTGCGCCGCAAGCGGAGATTGTCACGTTCTCTG
GCGTATGGGTTGGTTTCGCCAGGCACCTGGTAAAGAACGTGAATTTGTGG
CAGCCATTAGCCTCGACGGTTTGTTTACCAACTATGCCGATTCTGTTCGT
GGTCGCTTCAGCATTTCAGCAGATTCTGCGAAGAATACCGTGTACTTACA
GATGAACTCACTGAAACCTGAGGATACCGCCGTTTATTATTGTGCGGCAA
TGCCTTTTTTTGACTATTGGGGTCAGGGCACACAAGTCACGGTCTCAAGC
GGCGGAAGCTCTGGTGAACAGAAACTGATTTCAGAAGCCAA
DCH
VLWR#LDGLF#VQFNGTAVA#evqlvesggglvqagdslrlscaasgDCH
VLWRmgwfrqapgkerefvaaisLDGLFtnyadsvrcrfsisadsakntv
ylqmnslkpedtavyycnaVQFNGTAVAdywgqgtqvtfss#ACCATGGA
AGTTCAACTGGTTGAATCTGGTGGCGGTTTAGTTCAAGCCGGTGATAGCC
TTCGTCTGAGCTGCGCCGCAAGCGGAGATTGTCACGTTCTCTGGCGTATG
GGTTGGTTTCGCCAGGCACCTGGTAAAGAACGTGAATTTGTGGCAGCCAT
TAGCCTCGACGGTTTGTTTACCAACTATGCCGATTCTGTTCGTTGTCGCT
TCAGCATTTCAGCAGATTCTGCGAAGAATACCGTGTACTTACAGATGAAC
TCACTGAAACCTGAGGATACCGCCGTTTATTATTGTAATGCAGTGCAGTT
TAACGGGACGGCGGTTGCGGACTATTGGGGTCAGGGCACACAAGTCACGT
TCTCAAGCGGCGGAAGCTCTGGTGAACAGAAACTGATTTCAGAAGCCAA
DCHVLWR#LDGLF#MPFF#evqlvesggglvqagdslrlscaasg
DCHVLWRmgwfrqapskerefvaaisLDGLFtnyadsvrgrfsisadsak
ntvylqmnslkpedtavyycaaMPFFdywgqgtqvtvss#ACCATGGAAG
TTCAACTGGTTGAATCTGGTGGCGGTTTAGTTCAAGCCGGTGATAGCCTT
CGTCTGAGCTGCGCCGCAAGCGGAGATTGTCACGTTCTCTGGCGTATGGG
TTGGTTTCGCCAGGCACCTAGTAAAGAACGTGAATTTGTGGCAGCCATTA
GCCTCGACGGTTTGTTTACCAACTATGCCGATTCTGTTCGTGGTCGCTTC
AGCATTTCAGCAGATTCTGCGAAGAATACCGTGTACTTACAGATGAACTC
ACTGAAACCTGAGGATACCGCCGTTTATTATTGTGCGGCAATGCCTTTTT
TTGACTATTGGGGTCAGGGCACACAAGTCACGGTCTCAAGCGGCGGAAGC
TCTGGTGAACAGAAACTGATTTCAGAAGCCAA
DCHVLWR#LDGL
F#MPFF#evrlvesggglvqagdslrlscaaggDCHVLWRmgwfrqapgk
erefvaaisLDGLFtnyadsvrgrfsisadsakntvylqmnslkpedtav
yycaaMPFFdywgqgtqvtvss#ATATACCATGGAAGTTCGACTGGTTGA
ATCTGGTGGCGGTTTAGTTCAAGCCGGTGATAGCCTTCGTCTGAGCTGCG
CCGCAGGCGGAGATTGTCACGTTCTCTGGCGTATGGGTTGGTTTCGCCAG
GCACCTGGTAAAGAACGTGAATTTGTGGCAGCCATTAGCCTCGACGGTTT
GTTTACCAACTATGCCGATTCTGTTCGTGGTCGCTTCAGCATTTCAGCAG
ATTCTGCGAAGAATACCGTGTACTTACAGATGAACTCACTGAAACCTGAG
GATACCGCCGTTTATTATTGTGCGGCAATGCCTTTTTTTGACTATTGGGG
TCAGGGCACACAAGTCACGGTCTCAAGCGGCGGAAGCTCTGGTGAACAGA
AACTGATTTCAGAAGCCAA
DCHVLWR#LDGLF#LPATSG#evql
vesggglvhagdrlrlscaasgDCHVLWRmgwfrqapgkerefvaaisLD
GLFtnyadsvrgrfsisadsakntvylqmnslkpedtavyycaaLPATSG
dywgqgtqvtvss#GAAGGAGATATACCATGGAAGTTCAACTGGTTGAAT
CTGGTGGCGGTTTAGTTCACGCCGGTGATCGCCTTCGTCTGAGCTGCGCC
GCAAGCGGAGATTGTCACGTTCTCTGGCGTATGGGTTGGTTTCGCCAGGC
ACCTGGTAAAGAACGTGAATTTGTGGCAGCCATTAGCCTCGACGGTTTGT
TTACCAACTATGCCGATTCTGTTCGTGGTCGCTTCAGCATTTCGGCAGAT
TCTGCGAAGAATACCGTGTACTTACAGATGAACTCACTGAAACCTGAGGA
TACCGCCGTTTATTATTGTGCGGCATTGCCGGCGACTTCTGGCGACTATT
GGGGTCAGGGCACACAAGTCACGGTCTCAAGCGGCGGAAGCTCTGGTGAA
CAGAAACTGATTTCAGAAGCCAA
DCHVLWR#LDGLF#MPFF#ev
qlvesggglvqagdslrlscaasgDCHVLWRmgwfrqapakerefvaais
LDGLFtnyadsvrgrfsisadsakntvylqmnslkpedtavyycaaMPFF
dywgqgtqvtvss#ATATACCATGGAAGTTCAACTGGTTGAATCTGGTGG
CGGTTTAGTTCAAGCCGGTGATAGCCTTCGTCTGAGCTGCGCCGCAAGCG
GAGATTGTCACGTTCTCTGGCGTATGGGTTGGTTTCGCCAGGCACCTGCT
AAAGAACGTGAATTTGTGGCAGCCATTAGCCTCGACGGTTTGTTTACCAA
CTATGCCGATTCTGTTCGTGGTCGCTTCAGCATTTCAGCAGATTCTGCGA
AGAATACCGTGTACTTACAGATGAACTCACTGAAACCTGAGGATACCGCC
GTTTATTATTGTGCGGCAATGCCTTTTTTTGACTATTGGGGTCAGGGCAC
ACAAGTCACGGTCTCAAGCGGCGGAAGCTCTGGTGAACAGAAACTGATTT
CAGAAGCCAA
DCHVLWR#LDGLF#HHFHPGGMD#evqlvesggg
lvqagdslrlscaasgDCHVLWRmgwfrqapgkerefvaaisLDGLFtny
adsvrgrfsisadsakntvylqmnslkpedtavyycnaHHFHPGGMDdyw
gqgtqvavss#ATACCATGGAAGTTCAACTGGTTGAATCTGGTGGCGGTT
TAGTTCAAGCCGGTGATAGCCTTCGTCTGAGCTGCGCCGCAAGCGGAGAT
TGTCACGTTCTCTGGCGTATGGGTTGGTTTCGCCAGGCACCTGGTAAAGA
ACGTGAATTTGTGGCAGCCATTAGCCTCGACGGTTTGTTTACCAACTATG
CCGATTCTGTTCGTGGTCGCTTCAGCATTTCAGCAGATTCTGCGAAGAAT
ACCGTGTACTTACAGATGAACTCACTGAAACCTGAGGATACCGAACAGAA
ACTGATTTCAGAAGCCAA
DCHVLWR#LDGLF#MPFF#evqlves
ggglvqagdslrlscaasgDCHVLWRmgwfrqapgkerefvaaisLDGLF
tnyvdsvrgrfsisadsakntvylqmnslkpedtavyycaaMPFFdywgl
gtqvtvss#GGAGATATACCATGGAAGTTCAACTGGTTGAATCTGGTGGC
GGTTTAGTTCAAGCCGGTGATAGCCTTCGTCTGAGCTGCGCCGCAAGCGG
AGATTGTCACGTTCTCTGGCGTATGGGTTGGTTTCGCCAGGCACCTGGTA
AAGAACGTGAATTTGTGGCAGCCATTAGCCTCGACGGTTTGTTTACCAAC
TATGTCGATTCTGTTCGTGGTCGCTTCAGCATTTCAGCAGATTCTGCGAA
GAATACCGTGTACTTACAGATGAACTCACTGAAACCTGAGGATACCGCCG
TTTATTATTGTGCGGCAATGCCTTTTTTTGACTATTGGGGTCTGGGCACA
CAAGTCACGGTCTCAAGCGGCGGAAGCTCTGGTGAACAGAAACTGATTTC
AGAAGCCAA
DCHVLWR#LDGLF#HHFHPGGMD#evqlvesgggl
vhagdslrlscaasgDCHVLWRmgwfrqapgkerefvaaisLDGLFtnya
dsvrgrfsisadsakntvylqmnslkpedtavyycnaHHFHPGGMDdywg
qgtqvtvss#GAAGGAGATATACCATGGAAGTTCAACTGGTTGAATCTGG
TGGCGGTTTAGTTCACGCCGGTGATAGCCTTCGTCTGAGCTGCGCCGCAA
GCGGAGATTGTCACGTTCTCTGGCGTATGGGTTGGTTTCGCCAGGCACCT
GGTAAAGAACGTGAATTTGTGGCAGCCATTAGCCTCGACGGTTTGTTTAC
CAACTATGCCGATTCTGTTCGTGGTCGCTTCAGCATTTCAGCAGATTCTG
CGAAGAATACCGTGTACTTACAGATGAACTCACTGAAACCTGAGGATACC
GCCGTTTATTATTGTAATGCACACCATTTTCATCCTGGTGGTATGGATGA
CTATTGGGGTCAGGGCACACAAGTCACGGTCTCAAGCGGCGGAAGCTCTG
GTGAACAGAAACTGATTTCAGAAGCCAA
DCHVLWR#LDGLF#IP
FF#evqlvesggglvqagdslrlscaasgDCHVLWRmgwfrqapgkeref
vaaisLDGLFtnyadsvrgrfsisadsakntvylqmnslkpedtavyyca
alPFFdywgqgtqvtvss#ATATACCATGGAAGTTCAACTGGTTGAATCT
GGTGGCGGTTTAGTTCAAGCCGGTGATAGCCTTCGTCTGAGCTGCGCCGC
AAGCGGAGATTGTCACGTTCTCTGGCGTATGGGTTGGTTTCGCCAGGCAC
CTGGTAAAGAACGTGAATTTGTGGCAGCCATTAGCCTCGACGGTTTGTTT
ACCAACTATGCCGATTCTGTTCGTGGTCGCTTCAGCATTTCAGCAGATTC
TGCGAAGAATACCGTGTACTTACAGATGAACTCACTGAAACCTGAGGATA
CCGCCGTTTATTATTGTGCGGCAATTCCTTTTTTTGACTATTGGGGTCAG
GGCACACAAGTCACGGTCTCAAGCGGCGGAAGCTCTGGTGAACAGAAACT
GATTTCAGAAGCCAA
DCHVLWR#LDGLF#MPFF#evqlvesggg
lvqagdslrlgcaasgDCHVLWRmgwfrqapgkerefvaaisLDGLFtny
adsvrgrfsisadsakntvylqmnslkpedtavyycaaMPFFdywgqgtq
vtvss#ATACCATGGAAGTTCAACTGGTTGAATCTGGTGGCGGTTTAGTT
CAAGCCGGTGATAGCCTTCGTTTGGGCTGCGCCGCAAGCGGAGATTGTCA
CGTTCTCTGGCGTATGGGTTGGTTTCGCCAGGCACCTGGTAAAGAACGTG
AATTTGTGGCAGCCATTAGCCTCGACGGTTTGTTTACCAACTATGCCGAT
TCTGTTCGTGGTCGCTTCAGCATTTCAGCAGATTCTGCGAAGAATACCGT
GTACTTACAGATGAACTCACTGAAACCTGAGGATACCGCCGTTTATTATT
GTGCGGCAATGCCTTTTTTTGACTATTGGGGTCAGGGCACACAAGTCACG
GTCTCAAGCGGCGGAAGCTCTGGTGAACAGAAACTGATTTCAGAAGCCAA
DCHVLWR#LDGLF#LPSVCT#evqlvesggglvqagdslrlsca
asgDCHVLWRmgwfrqapgkerefvaaisLDGLFtnyadsvrgrfsisad
sakntvylqinslkpedtavyycaaLPSVCTdywvhctqvtvss#GAAGG
AGATATACCATGGAAGTTCAACTGGTTGAATCTGGTGGCGGTTTAGTTCA
AGCCGGTGATAGCCTTCGTCTGAGCTGCGCCGCAAGCGGAGATTGTCACG
TTCTCTGGCGTATGGGTTGGTTTCGCCAGGCACCTGGTAAAGAACGTGAA
TTTGTGGCAGCCATTAGCCTCGACGGTTTGTTTACCAACTATGCCGATTC
TGTTCGTGGTCGCTTCAGCATTTCAGCAGATTCTGCGAAGAATACCGTGT
ACTTACAGATTAACTCACTGAAACCTGAGGATACCGCCGTTTATTATTGT
GCGGCACTGCCTAGTGTGTGTACCGACTATTGGGTTCATTGCACACAAGT
CACGGTCTCAAGCGGCGGAAGCTCTGGTGAACAGAAACTGATTTCAGAAG
CCAA
DCHVLWR#LDGLF#LPSVCT#evqlvesggglvqagdslr
lscaasgDCHVLWRmgwfrqapgkerefvaaisLDGLFtnyadsvrgrfs
isadsakntvylqmnslkpedtavyycaaLPSVCTdywgkgtqvtvss#A
CCATGGAAGTTCAACTGGTTGAATCTGGTGGCGGTTTAGTTCAAGCCGGT
GATAGCCTTCGTCTGAGCTGCGCCGCAAGCGGAGATTGTCACGTTCTCTG
GCGTATGGGCTGGTTTCGCCAGGCACCTGGTAAAGAACGTGAATTTGTGG
CAGCCATTAGCCTCGACGGTTTGTTTACCAACTATGCCGATTCTGTTCGT
GGTCGCTTCAGCATTTCAGCAGATTCTGCGAAGAATACCGTGTACTTACA
GATGAACTCACTGAAACCTGAGGATACCGCCGTTTATTATTGTGCGGCAC
TGCCTAGTGTGTGTACCGACTATTGGGGGAAGGGCACACAAGTCACGGTC
TCAAGCGGCGGAAGCTCTGGTGAACAGAAAATGATTTCAGAAGCCAA
0
DCHVLWR#LDGLF#MPFF#evqlvesggglvqagdslrlscaasgDC
HVLWRmgwfrqapgkerefvaaisLDGLFtnyadsvrgrfsisadsaknt
vyfqmnslkpedtavyycaaMPFFdywgqgtqvtvss#AAGGAGATATAC
CATGGAAGTTCAACTGGTTGAATCTGGTGGCGGTTTAGTTCAAGCCGGTG
ATAGCCTTCGTCTGAGCTGCGCCGCAAGCGGAGATTGTCACGTTCTCTGG
CGTATGGGTTGGTTTCGCCAGGCACCTGGTAAAGAACGTGAATTTGTGGC
AGCCATTAGCCTCGACGGTTTGTTTACCAACTATGCCGATTCTGTTCGTG
GTCGCTTCAGCATTTCAGCAGATTCTGCGAAGAATACCGTGTACTTTCAG
ATGAACTCACTGAAACCTGAGGATACCGCCGTTTATTATTGTGCGGCAAT
GCCTTTTTTTGACTATTGGGGTCAGGGCACACAAGTCACGGTCTCAAGCG
GCGGAAGCTCTGGTGAACAGAAACTGATTTCAGAAGCCAA
DCHV
LWR#LDGLF#LPATSG#evqlvesggglvqagdslrlscaasgDCHVLWR
mgwfrqapgkerefvaaisLDGLFtnyadsvrgrfsisadsakntvylqi
nslkpedtavyycaaLPATSGdywgqgtqvtvss#GGAGATATACCATGG
AAGTTCAACTGGTTGAATCTGGTGGCGGTTTAGTTCAAGCCGGTGATAGC
CTTCGTCTGAGCTGCGCCGCAAGCGGAGATTGTCACGTTCTCTGGCGTAT
GGGTTGGTTTCGCCAGGCACCCGGTAAAGAACGTGAATTTGTGGCAGCCA
TTAGCCTCGACGGTTTGTTTACCAACTATGCCGATTCTGTTCGTGGTCGC
TTCAGCATTTCAGCAGATTCTGCGAAGAATACCGTGTACTTACAGATTAA
CTCACTGAAACCTGAGGATACCGCCGTTTATTATTGTGCGGCATTGCCGG
CGACTTCTGGCGACTATTGGGGTCAGGGCACACAAGTCACGGTCTCAAGC
GGCGGAAGCTCTGGTGAACAGAAACTGATTTCAGAAGCCAA
DCH
VLWR#LDGLF#MSQPTL#qvklqesggglvqagdslrlscaasgDCHVLW
RmgwfrqapgkerefvaaisLDGLFtnyadsvrgrfsisadsakntvylq
mnslkpedtavyycaaMSQPTLdywgqgtqvtvss#AGGAGATATACCAT
GCAGGTTAAACTGCAAGAATCTGGTGGCGGTTTAGTTCAAGCCGGTGATA
GCCTTCGTCTGAGCTGCGCCGCAAGCGGAGATTGTCACGTTCTCTGGCGT
ATGGGTTGGTTTCGCCAGGCACCTGGTAAAGAACGTGAATTTGTGGCAGC
CATTAGCCTCGACGGTTTGTTTACCAACTATGCCGATTCTGTTCGTGGTC
GCTTCAGCATTTCAGCAGATTCTGCGAAGAATACCGTGTACTTACAGATG
AACTCACTGAAACCTGAGGATACCGCCGTTTATTATTGTGCGGCAATGAG
TCAGCCTACCTTGGACTATTGGGGTCAGGGCACACAAGTCACGGTCTCAA
GCGGCGGAAGCTCTGGTGAACAGAAACTGATTTCAGAAGCCAA
D
CHVLWR#LDGLF#MPFF#evqlvesggglvqagdslrlscaasgDCHVLW
RmgwfrrapgkerefvaaisLDGLFtnyadsvrgrfsisadsakntvylq
mnslkpedtavyycaaMPFFdywgqgtqvtvss#GAAGGAGATATACCAT
GGAAGTTCAACTGGTTGAATCTGGTGGCGGTTTAGTTCAAGCCGGTGATA
GCCTTCGTCTGAGCTGCGCCGCAAGCGGAGATTGTCACGTTCTCTGGCGT
ATGGGTTGGTTTCGCCGGGCACCTGGTAAAGAACGTGAATTTGTGGCAGC
CATTAGCCTCGACGGTTTGTTTACCAACTATGCCGATTCTGTTCGTGGTC
GCTTCAGCATTTCAGCAGATTCTGCGAAGAATACCGTGTACTTACAGATG
AACTCACTGAAACCTGAGGATACCGCCGTTTATTATTGTGCGGCAATGCC
TTTTTTTGACTATTGGGGTCAGGGCACACAAGTCACGGTCTCAAGCGGCG
GAAGCTCTGGTGAACAGAAACTGATTTCAGAAGCCAA
DCHVLWRLDGLF#MPFF#evqrvesggglvkagdslslscaasgDCHVLW
Rmgwfr
qapgkerefvaaisLDGLFtnyadsvrgrfsisadsakntvylqmnslkp
edtavyycaaMPFFdywgqgtqvtvss#GGAGATATACCATGGAAGTTCA
ACGGGTTGAATCTGGTGGCGGTTTAGTTAAAGCCGGTGATAGCCTTAGTC
TGAGCTGCGCCGCAAGCGGAGATTGTCACGTTCTCTGGCGTATGGGTTGG
TTTCGCCAGGCACCTGGTAAAGAACGTGAATTTGTGGCAGCCATTAGCCT
CGACGGTTTGTTTACCAACTATGCCGATTCTGTTCGTGGTCGCTTCAGCA
TTTCAGCAGATTCTGCGAAGAATACCGTGTACTTACAGATGAACTCACTG
AAACCTGAGGATACCGCCGTTTATTATTGTGCGGCAATGCCTTTTTTTGA
CTATTGGGGTCAGGGCACACAAGTCACGGTCTCAAGCGGCGGAAGCTCTG
GTGAACAGAAACTGATTTCAGAAGCCAA
DCHVLWR#LDGLF#LP
ATSG#evqlvesggglvqdgdslrlscaasgDCHVLWRmgwfrqapgker
efvaaisLDGLFtnyadsvrgrfsisadsakntvylqmnslkpedtavyy
caaLPATSGdywgqgtqvtvss#ATATACCATGGAAGTTCAACTGGTTGA
ATCTGGTGGCGGTTTAGTTCAAGACGGTGATAGCCTTCGTCTGAGCTGCG
CCGCAAGCGGAGATTGTCACGTTCTCTGGCGTATGGGTTGGTTTCGCCAG
GCACCTGGTAAAGAACGTGAATTTGTGGCAGCCATTAGCCTCGACGGTTT
GTTTACCAACTATGCCGATTCTGTTCGTGGTCGCTTCAGCATTTCAGCAG
ATTCTGCGAAGAATACCGTGTACTTACAGATGAACTCACTGAAACCTGAG
GATACCGCCGTTTATTATTGTGCGGCATTGCCGGCGACTTCTGGCGACTA
TTGGGGTCAGGGCACACAAGTCACGGTCTCAAGCGGCGGAAGCTCTGGTG
AACAGAAACTGATTTCAGAAGCCAA
DCHVLWR#LDGLF#LPATS
G#evqlvesggglvqagdslrlscaasgDCHVLWRmgwfrqapgkerefv
aaisLDGLFtnyvdsvrgrfsisadsakntvylqmnslkpedtavyycaa
LPATSGdywgqgtqgtvss#ATACCATGGAAGTTCAACTGGTTGAATCTG
GTGGCGGTTTAGTTCAAGCCGGTGATAGCCTTCGTCTGAGCTGCGCCGCA
AGCGGAGATTGTCACGTTCTCTGGCGTATGGGTTGGTTTCGCCAGGCACC
TGGTAAAGAACGTGAATTTGTGGCAGCCATTAGCCTCGACGGTTTGTTTA
CCAACTATGTCGATTCTGTTCGTGGTCGCTTCAGCATTTCAGCAGATTCT
GCGAAGAATACCGTGTACTTACAGATGAACTCACTGAAACCTGAGGATAC
CGCCGTTTATTATTGTGCGGCATTGCCGGCGACTTCTGGCGACTATTGGG
GTCAGGGCACACAAGGCACGGTCTCAAGCGGCGGAAGCTCTGGTGAACAG
AAACTGATTTCAGAAGCCAA
DCHVLWR#LDGLF#MPFF#evqlv
esggglvqagdslrlscaasgDCHVLWRmgwfrqapgkerefvaaisLDG
LFtnyadsvrgrfsisadstkntvylqmnslkpedtavyycaaMPFFdyw
gqgtqgtvss#ATATACCATGGAAGTTCAACTGGTTGAATCTGGTGGCGG
TTTAGTTCAAGCCGGTGATAGCCTTCGTCTGAGCTGCGCCGCAAGCGGAG
ATTGTCACGTTCTCTGGCGTATGGGTTGGTTTCGCCAGGCACCTGGTAAA
GAACGTGAATTTGTGGCAGCCATTAGCCTCGACGGTTTGTTTACCAACTA
TGCCGATTCTGTTCGTGGTCGCTTCAGCATTTCAGCAGATTCTACGAAGA
ATACCGTGTACTTACAGATGAACTCACTGAAACCTGAGGATACCGCCGTT
TATTATTGTGCGGCAATGCCTTTTTTTGACTATTGGGGTCAGGGCACACA
AGGCACGGTCTCAAGCGGCGGAAGCTCTGGTGAACAGAAACTGATTTCAG
AAGCCAA
DCHVLWR#LDGLF#MPFF#qvqlvesggglvqagdsl
rlscaasgDCHVLWRmgwfrqapgkerefvaaisLDGLFtnyadsvrgrf
sisadsakntvylqmnslkpedtavyycaaMPFFdywgqgtqvtvss#AG
ATATACCATGCAGGTTCAACTGGTTGAATCTGGTGGCGGTTTAGTTCAAG
CCGGTGATAGCCTTCGTCTGAGCTGCGCCGCAAGCGGAGATTGTCACGTT
CTCTGGCGTATGGGTTGGTTTCGCCAGGCACCTGGTAAAGAACGTGAATT
TGTGGCAGCCATTAGCCTCGACGGTTTGTTTACCAACTATGCCGATTCTG
TTCGTGGTCGCTTCAGCATTTCAGCAGATTCTGCGAAGAATACCGTGTAC
TTACAGATGAACTCACTGAAACCTGAGGATACCGCCGTTTATTATTGTGC
GGCAATGCCTTTTTTTGACTATTGGGGTCAGGGCACACAAGTCACGGTCT
CAAGCGGCGGAAGCTCTGGTGAACAGAAACTGATTTCAGAAGCCAA
DCHVLWR#LDGLF#LPATSG#evqlvesggglvqagdslrlscaasgD
CHVLWRmgwfrqapgkehefvaaisLDGLFtnyadsvrgrfsisadsakn
tvylqmnslkpedtavyycaaLPATSGdywgqgtqgtvss#ATATACCAT
GGAAGTTCAACTGGTTGAATCTGGTGGCGGTTTAGTTCAAGCCGGTGATA
GCCTTCGTCTGAGCTGCGCCGCAAGCGGAGATTGTCACGTTCTCTGGCGT
ATGGGTTGGTTTCGCCAGGCACCTGGTAAAGAACATGAATTTGTGGCAGC
CATTAGCCTCGACGGTTTGTTTACCAACTATGCCGATTCTGTTCGTGGTC
GCTTCAGCATTTCAGCAGATTCTGCGAAGAATACCGTGTACTTACAGATG
AACTCACTGAAACCTGAGGATACCGCCGTTTATTATTGTGCGGCATTGCC
GGCGACTTCTGGCGACTATTGGGGTCAGGGCACACAAGGCACGGTCTCAA
GCGGCGGAAGCTCTGGTGAACAGAAACTGATTTCAGAAGCCAA
D
CHVLWR#LDGLF#LPSVCS#evqlvesggglvqagdslrlscdasgDCHV
LWRmgwfrqapgkerefvaaisLDGLFtnyadsvrgrfsisadsakntvy
lqmnslkpedtavyycaaLPSVCSdywgqgtqdtvss#ATACCATGGAAG
TTCAACTGGTTGAATCTGGTGGCGGTTTAGTTCAAGCCGGTGATAGCCTT
CGTCTGAGCTGCGACGCAAGCGGAGATTGTCACGTTCTCTGGCGTATGGG
TTGGTTTCGCCAGGCACCTGGTAAAGAACGTGAATTTGTGGCAGCCATTA
GCCTCGACGGTTTGTTTACCAACTATGCCGATTCTGTTCGTGGTCGCTTC
AGCATTTCAGCAGATTCTGCGAAGAATACCGTGTACTTACAGATGAACTC
ACTGAAACCTGAGGATACCGCCGTTTATTATTGTGCGGCACTGCCTAGTG
TGTGTTCCGACTATTGGGGGCAGGGCACACAAGACACGGTCTCAAGCGGC
GGAAGCTCTGGTGAACACAAACTGATTTCAGAAGCCAA
DCHVLW
R#LDGLF#MPFF#evqlvesggglvkagdslrlscaasgDCHVLWRmgwf
rqapgkerefvaaisLDGLFtnyadsvrgrfsisadsakntvylqmnslk
pedtavyycaaMPFFdywgqgtqgtvss#AGGAGATATACCATGGAAGTT
CAACTGGTTGAATCTGGTGGCGGTTTAGTTAAAGCCGGTGATAGCCTTCG
TCTGAGCTGCGCCGCAAGCGGAGATTGTCACGTTCTCTGGCGTATGGGTT
GGTTTCGCCAGGCACCTGGTAAAGAACGTGAATTTGTGGCAGCCATTAGC
CTCGACGGTTTGTTTACCAACTATGCCGATTCTGTTCGTGGTCGCTTCAG
CATTTCAGCAGATTCTGCGAAGAATACCGTGTACTTACAGATGAACTCAC
TGAAACCTGAGGATACCGCCGTTTATTATTGTGCGGCAATGCCTTTTTTT
GACTATTGGGGTCAGGGCACACAAGGCACGGTCTCAAGCGGCGGAAGCTC
TGGTGAACAGAAACTGATTTCAGAAGCCAA
DCHVLWR#LDGLF#
LPATSG#evqlvesggglvqagdslrlscaasgDCHVLWRmgwfrqapgk
erefvaaisLDGLFtnyadtvrgrfsisadsakntvylqmnslkpedtav
yycaaLPATSGdywgqgtqgpvss#ATACCATGGAAGTTCAACTGGTTGA
ATCTGGTGGCGGTTTAGTTCAAGCCGGTGATAGCCTTCGTCTGAGCTGCG
CCGCAAGCGGAGATTGTCACGTTCTCTGGCGTATGGGTTGGTTTCGCCAG
GCACCTGGTAAAGAACGTGAATTTGTGGCAGCCATTAGCCTCGACGGTTT
GTTTACCAACTATGCCGATACTGTTCGTGGTCGCTTCAGCATTTCAGCAG
ATTCTGCGAAGAATACCGTGTACTTACAGATGAACTCACTGAAACCTGAG
GATACCGCCGTTTATTATTGTGCGGCATTGCCGGCGACTTCTGGCGACTA
TTGGGGTCAGGGCACACAAGGCCCGGTCTCAAGCGGCGGAAGCCCTGGTG
AACAGAAACTGATTTCAGAAGCCAA
DCHVLWR#LDGLF#MPFF#
evqlvesggglvqagdslrlscaasgDCHVLWRmgwfrqapgkerefvaa
isLDGLFtnyadsvrgrfsisadsakntvylqmnslkpkdtavyycaaMP
FFdywgqgtqvtvss#GAAGGAGATATACCATGGAAGTTCAACTGGTTGA
ATCTGGTGGCGGTTTAGTTCAAGCCGGTGATAGCCTTCGTCTGAGTTGCG
CCGCAAGCGGAGATTGTCACGTTCTCTGGCGTATGGGTTGGTTTCGCCAG
GCACCTGGTAAAGAACGTGAATTTGTGGCAGCCATTAGCCTCGACGGTTT
GTTTACCAACTATGCCGATTCTGTTCGTGGTCGCTTCAGCATTTCAGCAG
ATTCTGCGAAGAATACCGTGTACTTACAGATGAACTCACTGAAACCTAAG
GATACCGCCGTTTATTATTGTGCGGCAATGCCTTTTTTTGACTATTGGGG
TCAGGGCACACAAGTCACGGTCTCAAGCGGCGGAAGCTCTGGTGAACAGA
AACTGATTTCAGAAGCCAA
DCHVLWR#LDGLF#LPKRTV#qvql
vesggalvqagdslrlscaasgDCHVLWRmgwfrqapgkerefvaaisLD
GLFtnyadsvrgrfsisadsakntvylqmnslkpedtavyycaaLPKRTV
dywgqgtqgtvss#ATATACCATGCAGGTTCAACTGGTTGAATCTGGTGG
CGCATTAGTTCAAGCCGGTGATAGCCTTCGTCTGAGCTGCGCCGCAAGCG
GAGATTGTCACGTTCTCTGGCGTATGGGTTGGTTTCGCCAGGCACCTGGT
AAAGAACGTGAATTTGTGGCAGCCATTAGCCTCGACGGTTTGTTTACCAA
CTATGCCGATTCTGTTCGTGGTCGCTTCAGCATTTCAGCAGATTCTGCGA
AGAATACCGTGTACTTACAGATGAACTCACTGAAACCTGAGGATACCGCC
GTTTATTATTGTGCGGCACTGCCTAAGCGGACTGTGGACTATTGGGGTCA
GGGCACACAAGGCACGGTCTCAAGCGGCGGAAGCTCTGGTGAACAGAAAC
TGATTTCAGAAGCCAA
DCHVLWR#LDGLF#LPKRTV#qvqlves
ggalvqagdslrlscaasgDCHVLWRmgwfrqapgkerefvaaisLDGLF
inyadsvrgrfsisadsakntvylqmnslkpedtavyycaaLPKRTVdyw
gqgtqvtvss#ACCATGCAGGTTCAACTGGTTGAATCTGGTGGCGCATTA
GTTCAAGCCGGTGATAGCCTTCGTCTGAGCTGCGCCGCAAGCGGAGATTG
TCACGTTCTCTGGCGTATGGGTTGGTTTCGCCAGGCACCTGGTAAAGAAC
GTGAATTTGTGGCAGCCATTAGCCTCGACGGTTTGTTTATCAACTATGCC
GATTCTGTTCGTGGTCGCTTCAGCATTTCAGCAGATTCTGCGAAGAATAC
CGTGTACTTACAGATGAACTCACTGAAACCTGAGGATACCGCCGTTTATT
ATTGTGCGGCACTGCCTAAGCGGACTGTGGACTATTGGGGTCAGGGCACA
CAAGTCACGGTCTCAAGCGGCGGAAGCTCTGGTGAACAGAAACTGATTTC
AGAAGCCAA
DCHVLWR#LDGLF#MPFF#evqlvesggglvqagd
glrlscaasgDCHVLWRmgwfrqapgkerefvaaisLDGLFtnyadsvrg
rfsisadsakntvylqmnslkpedtavyycaaMPFFdywgqgtqvtvss#
ATACCATGGAAGTTCAACTGGTTGAATCTGGTGGCGGTTTAGTTCAAGCC
GGTGATGGCCTTCGTCTGAGCTGCGCCGCAAGCGGAGATTGTCACGTTCT
CTGGCGTATGGGTTGGTTTCGCCAGGCACCTGGTAAAGAACGTGAATTTG
TGGCAGCCATTAGCCTCGACGGTTTGTTTACCAACTATGCCGATTCTGTT
CGTGGTCGCTTCAGCATTTCAGCAGATTCTGCGAAGAATACCGTGTACTT
ACAGATGAACTCACTGAAACCTGAGGATACCGCCGTTTATTATTGTGCGG
CAATGCCTTTTTTTGACTATTGGGGTCAGGGCACACAAGTCACGGTCTCA
AGCGGCGGAAGCTCTGGTGAACAGAAACTGATTTCAGAAGCCAA
DCHVLWR#LDGLF#HHFHPGGMD#evllvesggglvqagdslrlscaasg
DCHVLWRmgwfrqapgkerefvaaisLDGLFtnyadsvrgrfsisadsak
ntvylqmnslkpedtavyycnaHHFHPGGMDdywgqgtqvtvss#GAAGG
AGATATACCATGGAAGTTCTACTGGTTGAATCTGGTGGCGGTTTAGTTCA
AGCCGGTGATAGCCTTCGTCTGAGCTGCGCCGCAAGCGGAGATTGTCACG
TTCTCTGGCGTATGGGTTGGTTTCGCCAGGCACCTGGTAAAGAACGTGAA
TTTGTGGCAGCCATTAGCCTCGACGGTTTGTTTACCAACTATGCCGATTC
TGTTCGTGGTCGCTTCAGCATTTCAGCAGATTCTGCGAAGAATACCGTGT
ACTTACAGATGAACTCACTGAAACCTGAGGATACCGCCGTTTATTATTGT
AATGCACACCATTTTCATCCTGGTGGTATGGATGACTATTGGGGTCAGGG
CACACAAGTCACGGTCTCAAGCGGCGGAAGCTCTGGTGAACAGAAACTGA
TTTCAGAAGCCAA
DCHVLWR#LDGLF#MPFF#evqlvesggglv
qagdslrlscaasgDCHVLWRmgwfrqapgkerefvaaisLDGLFtnyad
svrgrfsisadsakntvylqmnslkpentavyycaaMPFFdywgqgtqvt
vss#GGAGATATACCATGGAAGTTCAACTGGTTGAATCTGGTGGCGGTTT
AGTTCAAGCCGGTGATAGCCTTCGTCTGAGCTGCGCCGCAAGCGGAGATT
GTCACGTTCTCTGGCGTATGGGTTGGTTTCGCCAGGCACCTGGTAAAGAA
CGTGAATTTGTGGCAGCCATTAGCCTCGACGGTTTGTTTACCAACTATGC
CGATTCTGTTCGTGGTCGCTTCAGCATTTCAGCAGATTCTGCGAAGAATA
CCGTGTACTTACAGATGAACTCACTGAAACCTGAGAATACCGCCGTTTAT
TATTGTGCGGCAATGCCTTTTTTTGACTATTGGGGTCAGGGCACACAAGT
CACGGTCTCAAGCGGCGGAAGCTCTGGTGAACAGAAACTGATTTCAGAAG
CCAA
DCHVLWR#LDGLF#LPSVCl#evqlvesggglvqagdslr
lscaasgDCHVLWRmgwfrqapgkerefvaaisLDGLFtnyadsvrgrfs
isadsakntvylqmnslkpedtavyycaaLPSVCldywgqgtqvtvss#A
TACCATGGAAGTTCAACTGGTTGAATCTGGTGGCGGTTTAGTTCAAGCCG
GTGATAGCCTTCGTCTGAGCTGCGCCGCAAGCGGAGATTGTCACGTTCTC
TGGCGTATGGGTTGGTTTCGCCAGGCACCTGGTAAAGAACGTGAATTTGT
GGCAGCCATTAGCCTCGACGGTTTGTTTACCAACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGGC
ACTGCCTAGTGTGTGTATCGACTATTGGGGTCAGGGCACACAAGTCACGG
TCTCAAGCGGCGGAAGCTCTGGTGAACAGAAACTGATTTCAGAAGCCAA
DCHVLWR#LDGLF#MPFF#evqlvesggglvqagdslrlscaasg
DCHVLWRmgwfrqapgkerefvaaisLDGLFtnyadsvrgrfsisadsak
ntvylqmnslkpedtavyycaaMPFFyywgqgtqvtvss#GAAGGAGATA
TACCATGGAAGTTCAACTGGTTGAATCTGGTGGCGGTTTAGTTCAAGCCG
GTGATAGCCTTCGTCTGAGCTGCGCCGCAAGCGGAGATTGTCACGTTCTC
TGGCGTATGGGTTGGTTTCGCCAGGCACCTGGTAAAGAACGTGAATTTGT
GGCAGCCATTAGCCTCGACGGTTTGTTTACCAACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGGC
AATGCCTTTTTTTTACTATTGGGGTCAGGGCACACAAGTCACGGTCTCAA
GCGGCGGAAGCTCTGGTGAACAGAAACTGATTTCAGAAGCCAA
D
CHVLWR#LDGLF#HHFHPGGMD#evqlvesggglvqagdslrlscaasgD
CHVLWRmgwfrqapgkerefvaaisLDGLFtnyadsvrgrfsfsadsakn
tvylqmnslkpedtavyycnaHHFHPGGMDdywgqgtqvtvss#ACCATG
GAAGTTCAACTGGTTGAATCTGGTGGCGGTTTAGTTCAAGCCGGTGATAG
CCTTCGTCTGAGCTGCGCCGCAAGCGGAGATTGTCACGTTCTCTGGCGTA
TGGGTTGGTTTCGCCAGGCACCTGGTAAAGAACGTGAATTTGTGGCAGCC
ATTAGCCTCGACGGTTTGTTTACCAACTATGCCGATTCTGTTCGTGGTCG
CTTCAGCTTTTCAGCAGATTCTGCGAAGAATACCGTGTACTTACAGATGA
ACTCACTGAAACCTGAGGATACCGCCGTTTATTATTGTAATGCACACCAT
TTTCATCCTGGTGGTATGGATGACTATTGGGGTCAGGGCACACAAGTCAC
GGTCTCAAGCGGCGGAAGCTCTGGTGAACAGAAACTGATTTCAGAAGCCA
A
DCHVLWR#LDGLF#HHFHPGGMD#evqlvesggglvhagdrlr
lscaasgDCHVLWRmgwfrqapgkerefvaaisLDGLFtnyadsvrgrfs
isadsakntvylqmnslkpedtavyycnaHHFHPGGMDdywgqgtqvtvs
s#GAAGGAGATATACCATGGAAGTTCAACTGGTTGAATCTGGTGGCGGTT
TAGTTCACGCCGGTGATCGCCTTCGTCTGAGCTGCGCCGCAAGCGGAGAT
TGTCACGTTCTCTGGCGTATGGGTTGGTTTCGCCAGGCACCTGGTAAAGA
ACGTGAATTTGTGGCAGCCATTAGCCTCGACGGTTTGTTTACCAACTATG
CCGATTCTGTTCGTGGTCGCTTCAGCATTTCAGCAGATTCTGCGAAGAAT
ACCGTGTACTTACAGATGAACTCACTGAAACCTAGCTCTGGTGAACAGAA
ACTGATTTCAGAAGCCAA
DCHVLWR#LDGLF#MPFF#evqlves
ggglvqagdslslscaasgDCHVLWRmgwfrqapgkerefvaaisLDGLF
tnyadsvrgrfsisadsakntvylqmnslkpedtavyycaaMPFFdywgq
gtqvtvss#GGAGATATACCATGGAAGTTCAACTGGTTGAATCTGGTGGC
GGTTTAGTTCAAGCCGGTGATAGCCTTAGTCTGAGCTGCGCCGCAAGCGG
AGATTGTCACGTTCTCTGGCGTATGGGTTGGTTTCGCCAGGCACCTGGTA
AAGAACGTGAATTTGTGGCAGCCATTAGCCTCGACGGTTTGTTTACCAAC
TATGCCGATTCTGTTCGTGGTCGCTTCAGCATTTCAGCAGATTCTGCGAA
GAATACCGTGTACTTACAGATGAACTCACTGAAACCTGAGGATACCGCCG
TTTATTATTGTGCGGCAATGCCTTTTTTTGACTATTGGGGTCAGGGCACA
CAAGTCACGGTCTCAAGCGGCGGAAGCTCTGGTGAACAGAAACTGATTTC
AGAAGCCAA
DCHVLWR#LDGLF#MPFF#evqlvesggglvqagd
slrlscaasgDCHVLWRmgwfrqapgkerefvaaisLDGLFtnyadsvrg
rfsisadsakntvylqmnslkpedtavyycaaMPFFdywghgtqvtvss#
ATACCATGGAAGTTCAACTGGTTGAATCTGGTGGCGGTTTAGTTCAAGCC
GGTGATAGCCTTCGTCTGAGCTGCGCCGCAAGCGGAGATTGTCACGTTCT
CTGGCGTATGGGTTGGTTTCGCCAGGCACCTGGTAAAGAACGTGAATTTG
TGGCAGCCATTAGCCTCGACGGTTTGTTTACCAACTATGCCGATTCTGTT
CGTGGTCGCTTCAGCATTTCAGCAGATTCTGCGAAGAATACCGTGTACTT
ACAGATGAACTCACTGAAACCTGAGGATACCGCCGTTTATTATTGTGCGG
CAATGCCTTTTTTTGACTATTGGGGGCATGGCACACAAGTCACGGTCTCA
AGCGGCGGAAGCTCTGGTGAACAGAAACTGATTTCAGAAGCCAA
DCHVLWR#LDGLF#HHFHPGGMD#evqlvqsggglvqagdslrlscaasg
DCHVLWRmgwfrqapgkerefvaaisLDGLFtnyadsvrgrfsisadsak
ntvylqmnslkpedtavyycnaHHFHPGGMDdywgqgtqvtvss#GAAGG
AGATATACCATGGAAGTTCAACTGGTTCAATCTGGTGGCGGTTTAGTTCA
AGCCGGTGATAGCCTTCGTCTGAGCTGCGCCGCAAGCGGAGATTGTCACG
TTCTCTGGCGTATGGGTTGGTTTCGCCAGGCACCTGGTAAAGAACGTGAA
TTTGTGGCAGCCATTAGCCTCGACGGTTTGTTTACCAACTATGCCGATTC
TGTTCGTGGTCGCTTCAGCATTTCAGCAGATTCTGCGAAGAATACCGTGT
ACTTACAGATGAACTCACTGAAACCTAGCTCTGGTGAACAGAAACTGATT
TCAGAAGCCAA
DCHVLWR#LDGLF#MPFF#evqlvesggglvqa
gdslrlscaasgDCHVLWRmgwfrqapgkerefvaaisLDGLFtnyadsv
rgrfsistdsakntvylqmnslkpedtavyycaaMPFFdywgqgtqvtvs
s#GGAGATATACCATGGAAGTTCAACTGGTTGAATCTGGTGGCGGTTTAG
TTCAAGCCGGTGATAGCCTTCGTCTGAGCTGCGCCGCAAGCGGAGATTGT
CACGTTCTCTGGCGTATGGGTTGGTTTCGCCAGGCACCTGGTAAAGAACG
TGAATTTGTGGCAGCCATTAGCCTCGACGGTTTGTTTACCAACTATGCCG
ATTCTGTTCGTGGTCGCTTCAGCATTTCAACAGATTCTGCGAAGAATACC
GTGTACTTACAGATGAACTCACTGAAACCTGAGGATACCGCCGTTTATTA
TTGTGCGGCAATGCCTTTTTTTGACTATTGGGGTCAGGGCACACAAGTCA
CGGTCTCAAGCGGCGGAAGCTCTGGTGAACAGAAACTGATTTCAGAAGCC
AA
DCHVLWR#LDGLF#MPFF#evqlvesgggsvqagdslrlsca
asgDCHVLWRmgwfrqapgkerefvaaisLDGLFtnyadsvrgrfsisad
sakntvylqmnslkpedtavyycaaMPFFdywgqgtqvtvss#GGAGATA
TACCATGGAAGTTCAACTGGTTGAATCTGGTGGCGGTTCAGTTCAAGCCG
GTGATAGCCTTCGTCTGAGCTGCGCCGCAAGCGGAGATTGTCACGTTCTC
TGGCGTATGGGTTGGTTTCGCCAGGCACCTGGTAAAGAACGTGAATTTGT
GGCAGCCATTAGCCTCGACGGTTTGTTTACCAACTATGCCGATTCTGTTC
GTGGTCGCTTCAGCATTTCAGCAGATTCTGCGAAGAATACCGTGTACTTA
CAGATGAACTCACTGAAACCTGAGGATACCGCCGTTTATTATTGTGCGGC
AATGCCTTTTTTTGACTATTGGGGTCAGGGCACACAAGTCACGGTCTCAA
GCGGCGGAAGCTCTGGTGAACAGAAACTGATTTCAGAAGCCAA
D
CHVLWR#LDGLF#LIPRPT#evqlvesggglvqagdslrlscaasgDCHV
LWRmgwfrqapgkerefvaaisLDGLFtnyadsvrgrfsisadsakntvy
lqmnslkpedtavyycaaLIPRPTdywgqctqvtvss#AAGGAGATATAC
CATGGAAGTTCAACTGGTTGAATCTGGTGGCGGTTTAGTTCAAGCCGGTG
ATAGCCTTCGTCTGAGCTGCGCCGCAAGCGGAGATTGTCACGTTCTCTGG
CGTATGGGTTGGTTTCGCCAGGCACCTGGTAAAGAACGTGAATTTGTGGC
AGCCATTAGCCTCGACGGTTTGTTTACCAACTATGCCGATTCTGTTCGTG
GTCGCTTCAGCATTTCAGCAGATTCTGCGAAGAATACCGTGTACTTACAG
ATGAACTCACTGAAACCTGAGGATACCGCCGTTTATTATTGTGCGGCACT
TATCCCGCGTCCGACCGACTATTGGGGTCAGTGCACACAAGTCACGGTCT
CAAGCGGCGGAAGCTCTGGTGAACAGAAACTGATTTCAGAAGCCAA

TABLE 5
Sequences belonging to cluster SR8. Each line in the table represents one sequence,
both segments and full length of the sequence are shown. Shown items are divided by “#” and in
the order from start to end of each line is: CDR1 amino acid sequence, CDR2 amino acid sequence,
CDR3 amino acid sequence, full-length amino acid sequence with CDRs capitalized, full-length
DNA sequence.
(SEQ ID NOS: 1421-2030)

DYCMDCF#PYSFK#PLNIGTATIA#evqlvesggglvkagdslrlscaasgDYCMDCFmgwfrqapgkerefvaaitPYSFKtyyadsvkgrftisrddarntvylqmnslkpedtavyycaa
PLNIGTATIAaywgqgtqvtvss#AGGAGATATACCATGGAAGTTCAACTGGTTGAATCTGGTGGCGGTTTAGTTAAAGCCGGTGATAGCCTTCGTCTGAGCTGC
GCCGCAAGCGGAGATTACTGCATGGATTGCTTTATGGGTTGGTTTCGCCAGGCACCTGGTAAAGAACGTGAATTTGTGGCAGCCATTACCCCCTATTCCTTT
AAGACCTACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGGCACCCCTCAATATCGGCACCGCGACGATTGCCGCCTATTGGGGTCAGGGCACACAAGTCACGGTCTCAAGCGGCGGAAGC
TCTGGTGAACAGAAACTGATTTCAGAAG
DYCMDCF#PYSFK#NPLDPNWTNS#evqlvesggglvqagdslrlscaasgDYCMDCFmgwfrqapgkerefvaaitPYSFKtyyadsvkgrftisrddarntvylqmnslkpedtavyyc
aaNPLDPNWTNSdywgqgtqvtvss#GGAGATATACCATGGAAGTTCAACTGGTTGAATCTGGTGGCGGTTTAGTTCAAGCCGGTGATAGCCTTCGTCTGAGCT
GCGCCGCAAGCGGAGATTACTGCATGGATTGCTTTATGGGTTGGTTTCGCCAGGCACCTGGTAAAGAACGTGAATTTGTGGCAGCCATTACCCCCTATTCCT
TTAAGACCTACTATGCCGATTCTGTTAAAGGTCGCTTCACTATTTCACGCGATGATGCGCGTAATACCGTGTACTTACAGATGAACTCACTGAAACCTGAGGA
TACCGCCGTTTATTATTGTGCGGCAAACCCTCTGGACCCCAACTGGACTAATTCTGACTATTGGGGTCAGGGCACACAAGTCACGGTCTCAAGCGGCGGAAG
CTCTGGTGAACAGAAACTGATTTCAGAAG
DYCMDCF#PYSFK#DSLILANSQW#evqlvesggglvqagdslrlscaasgDYCMDCFmgwfrqapgkerefvaaitPYSFKtyyadsvkgrftisrddarntvylqmnslkpedtavyyca
aDSLILANSQWdywgqgtqvtvss#AGATATACCATGGAAGTTCAACTGGTTGAATCTGGTGGCGGTTTAGTTCAAGCCGGTGATAGCCTTCGTCTGAGCTGCG
CCGCAAGCGGAGATTACTGCATGGATTGCTTTATGGGTTGGTTTCGCCAGGCACCTGGTAAAGAACGTGAATTTGTGGCAGCCATTACCCCCTATTCCTTTA
AGACCTACTATGCCGATTCTGTTAAAGGTCGCTTCACTATTTCACGCGATGATGCGCGTAATACCGTGTACTTACAGATGAACTCACTGAAACCTGAGGATAC
CGCCGTTTATTATTGTGCGGCAGACAGCCTTATTTTGGCTAATTCTCAGTGGGACTATTGGGGTCAGGGCACACAAGTCACGGTCTCAAGCGGCGGAAGCTC
TGGTGAACAGAAACTGATTTCAGAAG
DYCMDCF#PYSFK#VPNVTWSERA#evqlfesggglvqagdslrlscaasgDYCMDCFmgwfrqapgkerefvaaitPYSFKtyyadsvkgrftisrddarntvylqmnslkpedtavyyc
aaVPNVTWSERAdywgqgtqvtvss#AGATATACCATGGAAGTTCAACTGTTTGAATCTGGTGGCGGTTTAGTTCAAGCCGGTGATAGCCTTCGTCTGAGCTGC
GCCGCAAGCGGAGATTACTGCATGGATTGCTTTATGGGTTGGTTTCGCCAGGCACCTGGTAAAGAACGTGAATTTGTGGCAGCCATTACCCCCTATTCCTTT
AAGACCTACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGGCAGTCCCCAATGTGACCTGGTCCGAGAGGGCTGACTATTGGGGTCAGGGCACACAAGTCACGGTCTCAAGCGGCGGAAGC
TCTGGTGAACAGAAACTGATTTCAGAAG
DYCMDCF#PYSFK#PLNIGTATIA#evqlvesggglvqagdslrlscaasgDYCMDCFmgwfrqapgkerefvaaitPYSFKtyyadsvkgrftisrddarntvylqmnslkpedtavyycaa
PLNIGTATIAdywgqgtqvtvss#AGGAGATATACCATGGAAGTTCAACTGGTTGAATCTGGTGGCGGTTTAGTTCAAGCCGGTGATAGCCTTCGTCTGAGCTGC
GCCGCAAGCGGAGATTACTGCATGGATTGCTTTATGGGTTGGTTTCGCCAGGCACCTGGTAAAGAACGTGAATTTGTGGCAGCCATTACCCCCTATTCCTTT
AAGACCTACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGGCACCCCTCAATATCGGCACCGCGACGATTGCCGACTATTGGGGTCAGGGCACACAAGTCACGGTCTCAAGCGGCGGAAGC
TCTGGTGAACAGAAACTGATTTCAGAAG
DYCMDCF#PYSFK#PLNIGTATIA#evqlvesggglvqagdslrlscaasgDYCMDCFmgwfrqapgkerefvaaitPYSFKtyyadsvkgrftisrddarntvylqmnslkpedtavyycaa
PLNIGTATIAdywgqgtqvtiss#ACCATGGAAGTTCAACTGGTTGAATCTGGTGGCGGTTTAGTTCAAGCCGGTGATAGCCTTCGTCTGAGCTGCGCCGCAAGC
GGAGATTACTGCATGGATTGCTTTATGGGTTGGTTTCGCCAGGCACCTGGTAAAGAACGTGAATTTGTGGCAGCCATTACCCCCTATTCCTTTAAGACCTACT
ATGCCGATTCTGTTAAAGGTCGCTTCACTATTTCACGCGATGATGCGCGTAATACCGTGTACTTACAGATGAACTCACTGAAACCTGAGGATACCGCCGTTTA
TTATTGTGCGGCACCCCTCAATATCGGCACCGCGACGATTGCCGACTATTGGGGTCAGGGCACACAAGTCACGATCTCAAGCGGCGGAAGCTCTGGTGAAC
AGAAACTGATTTCAGAAG
DYCMDCF#PYSFK#VPNVTWSERA#evqlvesggglvqagdslrlscaasgDYCMDCFmgwfrqapgkerefvaaitPYSFKtyyadsvkgrftisrddarntvylqmnslkpedtavyyc
aaVPNVTWSERAaywgqgtqvtvss#AGATATACCATGGAAGTTCAACTGGTTGAATCTGGTGGCGGTTTAGTTCAAGCCGGTGATAGCCTTCGTCTGAGCTGC
GCCGCAAGCGGAGATTACTGCATGGATTGCTTTATGGGTTGGTTTCGCCAGGCACCTGGTAAAGAACGTGAATTTGTGGCAGCCATTACCCCCTATTCCTTT
AAGACCTACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGGCAGTCCCCAATGTGACCTGGTCCGAGAGGGCTGCCTATTGGGGTCAGGGCACACAAGTCACGGTCTCAAGCGGCGGAAGC
TCTGGTGAACAGAAACTGATTTCAGAAG
DYCMDCF#PYSFK#PLNIGTATIA#evqlvesggglvqtgdslrlscaasgDYCMDCFmgwfrqapgkerefvaaitPYSFKtyyadsvkgrftisrddarntvylqmnslkpedtavyycaa
PLNIGTATIAdywgqgtqvtvss#AGATATACCATGGAAGTTCAACTGGTTGAATCTGGTGGCGGTTTAGTTCAAACCGGTGATAGCCTTCGTCTGAGCTGCGCC
GCAAGCGGAGATTACTGCATGGATTGCTTTATGGGTTGGTTTCGCCAGGCACCTGGTAAAGAACGTGAATTTGTGGCAGCCATTACCCCCTATTCCTTTAAG
ACCTACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTGCGGCACCCCTCAATATCGGCACCGCGACGATTGCCGACTATTGGGGTCAGGGCACACAAGTCACGGTCTCAAGCGGCGGAAGCTCT
GGTGAACAGAAACTGATTTCAGAAG
DYCMDCF#PYSFK#NPLDPNWTNS#evqlvesggglvqagdslrlscaasgDYCMDCFmgwfrqapgkerefvaaitPYSFKtyyadsvkgrftisrddarntvylqmnslkpedtavyyc
aaNPLDPNWTNSaywgqgtqvtvss#ATATACCATGGAAGTTCAACTGGTTGAATCTGGTGGCGGTTTAGTTCAAGCCGGTGATAGCCTTCGTCTGAGCTGCGC
CGCAAGCGGAGATTACTGCATGGATTGCTTTATGGGTTGGTTTCGCCAGGCACCTGGTAAAGAACGTGAATTTGTGGCAGCCATTACCCCCTATTCCTTTAA
GACCTACTATGCCGATTCTGTTAAAGGTCGCTTCACTATTTCACGCGATGATGCGCGTAATACCGTGTACTTACAGATGAACTCACTGAAACCTGAGGATACC
GCCGTTTATTATTGTGCGGCAAACCCTCTGGACCCCAACTGGACTAATTCTGCCTATTGGGGTCAGGGCACACAAGTCACGGTCTCAAGCGGCGGAAGCTCT
GGTGAACAGAAACTGATTTCAGAAG
DYCMDCF#PYSFK#NPLDPNWTNS#evqlvesggglvqagdslrlscaasgDYCMDCFmgwfrqapgkerefvaaitPYSFKtyyadsvkgrftisrddarntvylqmnslkpedtavyyc
aaNPLDPNWTNSdywcqgtqvtvss#AAGGAGATATACCATGGAAGTTCAACTGGTTGAATCTGGTGGCGGTTTAGTTCAAGCCGGTGATAGCCTTCGACTGA
GCTGCGCCGCAAGCGGAGATTACTGCATGGATTGCTTTATGGGTTGGTTTCGCCAGGCACCTGGTAAAGAACGTGAATTTGTGGCAGCCATTACCCCCTATT
CCTTTAAGACCTACTATGCCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGGCAAACCCTCTGGACCCCAACTGGACTAATTCTGACTATTGGTGTCAGGGCACACAAGTCACGGTCTCAAGCGGCGG
AAGCTCTGGTGAACAGAAACTGATTTCAGAAG
DYCMDCF#PYSFK#NPLDPNWTNS#evqlvesggglvqagdslrlscaaggDYCMDCFmgwfrqapgkerefvaaitPYSFKtyyadsvkgrftisrddarntvylqmnslkpedtavyy
caaNPLDPNWTNSdywgqgtqvtvss#AGATATACCATGGAAGTTCAACTGGTTGAATCTGGTGGCGGTTTAGTTCAAGCCGGTGATAGCCTTCGTCTGAGCTG
CGCCGCAGGCGGAGATTACTGCATGGATTGCTTTATGGGTTGGTTTCGCCAGGCACCTGGTAAAGAACGTGAATTTGTGGCAGCCATTACCCCCTATTCCTT
TAAGACCTACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTGCGGCAAACCCTCTGGACCCCAACTGGACTAATTCTGACTATTGGGGTCAGGGCACACAAGTCACGGTCTCAAGCGGCGGAAG
CTCTGGTGAACAGAAACTGATTTCAGAAG
DYCMDCF#PYSFK#SMHLNNISIS#evqlvesggglvqagdslrlscaasgDYCMDCFmgwfrqapgkerefvaaitPYSFKtyyadsvkgrftisrddarntvylqmnslkpedtavyyca
aSMHLNNISISdywgqgtqvtvss#AGGAGATATACCATGGAAGTTCAACTGGTTGAATCTGGTGGCGGTTTAGTTCAAGCCGGTGATAGCCTTCGTCTGAGCTG
CGCCGCAAGCGGAGATTACTGCATGGATTGCTTTATGGGTTGGTTTCGCCAGGCACCTGGTAAAGAACGTGAATTTGTGGCAGCCATTACCCCCTATTCCTT
TAAGACCTACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTGCGGCATCTATGCACCTGAACAATATCAGCATTAGTGACTATTGGGGTCAGGGCACACAAGTCACGGTCTCAAGCGGCGGAAG
CTCTGGTGAACAGAAACTGATTTCAGAAG
DYCMDCF#PYSFK#SANILHLSVI#evqlvesggglvqagdslrlscaasgDYCMDCFmgwfrqapgkerefvaaitPYSFKtyyadsvkgrftisrddarntvylqmnslkpedtavyycaa
SANILHLSVInywgqgtqvtvss#ATACCATGGAAGTTCAACTGGTTGAATCTGGTGGCGGTTTAGTTCAAGCCGGTGATAGCCTTCGTCTGAGCTGCGCCGCAA
GCGGAGATTACTGCATGGATTGCTTTATGGGTTGGTTTCGCCAGGCACCTGGTAAAGAACGTGAATTTGTGGCAGCCATTACCCCCTATTCCTTTAAGACCTA
CTATGCCGATTCTGTTAAAGGTCGCTTCACTATTTCACGCGATGATGCGCGTAATACCGTGTACTTACAGATGAACTCACTGAAACCTGAGGATACCGCCGTT
TATTATTGTGCGGCAAGCGCGAACATTCTCCATTTGTCTGTTATCAACTATTGGGGTCAGGGCACACAAGTCACGGTCTCAAGCGGCGGAAGCTCTGGTGAA
CAGAAACTGATTTCAGAAG
DYCMDCF#PYSFK#LLPLDGFETS#evqlvesggglvqagdslrlscaasgDYCMDCFmgwfrqapgkerefvaaitPYSFKtyyadsvkgrftisrddarntvylqmnslkpedtavyycaa
LLPLDGFETSdywgqgtqvtvss#ACCATGGAAGTTCAACTGGTTGAATCTGGTGGCGGTTTAGTTCAAGCCGGTGATAGCCTTCGTCTGAGCTGCGCCGCAAGC
GGAGATTACTGCATGGATTGCTTTATGGGTTGGTTTCGCCAGGCACCTGGTAAAGAACGTGAATTTGTGGCAGCCATTACCCCCTATTCCTTTAAGACCTACT
ATGCCGATTCTGTTAAAGGTCGCTTCACTATTTCACGCGATGATGCGCGTAATACCGTGTACTTACAGATGAACTCACTGAAACCTGAGGATACCGCCGTTTA
TTATTGTGCGGCACTTCTCCCCCTGGATGGTTTTGAGACTTCGGACTATTGGGGTCAGGGCACACAAGTCACGGTCTCAAGCGGCGGAAGCTCTGGTGAAC
AGAAACTGATTTCAGAAG
DYCMDCF#PYSFK#NPLDPNWTNS#evqlvesggglvqagdslrlscaasgDYCMDCFmgwfrqapgkerefvaaitPYSFKtyyadsvkgrstisrddarntvylqmnslkpedtavyy
caaNPLDPNWTNSaywghgtqvtvss#GGAGATATACCATGGAAGTTCAACTGGTTGAATCTGGTGGCGGTTTAGTTCAAGCCGGTGATAGCCTTCGTCTGAGC
TGCGCCGCAAGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTCCACTATTTCACGCGATGATGCGCGTAATACCGTGTACTTACAGATGAACTCACTGAAACCTGAGG
ATACCGCCGTTTATTATTGTGCGGCAAACCCTCTGGACCCCAACTGGACTAATTCTGCCTATTGGGGTCACGGCACACAAGTCACGGTCTCAAGCGGCGGAA
GCTCTGGTGAACAGAAACTGATTTCAGAAG
DYCMDCF#PYSFK#LLPLDGFETS#evqlvesggglvqagdrlrlscaasgDYCMDCFmgwfrqapgkerefvaaitPYSFKtyyadsvkgrftisrdgarntvylqmnslkpedtavyycaa
LLPLDGFETSdywgqgtqvtvss#ATACCATGGAAGTTCAACTGGTTGAATCTGGTGGCGGTTTAGTTCAAGCCGGTGATAGGCTTCGTCTGAGCTGCGCCGCAA
GCGGAGATTACTGCATGGATTGCTTTATGGGTTGGTTTCGCCAGGCACCTGGTAAAGAACGTGAATTTGTGGCAGCCATTACCCCCTATTCTTTTAAGACCTA
CTATGCCGATTCTGTTAAAGGTCGCTTCACTATTTCACGCGATGGTGCGCGTAATACCGTGTACTTACAGATGAACTCACTGAAACCTGAGGATACCGCCGTT
TATTATTGTGCGGCACTTCTCCCCCTGGATGGTTTTGAGACTTCGGACTATTGGGGTCAGGGCACACAAGTCACGGTCTCAAGCGGCGGAAGCTCTGGTGAA
CAGAAACTGATTTCAGAAG
DYCMDCF#PYSFK#TYYSTTCLLF#evqlvesggglvqagdslrlscaasgDYCMDCFmgwfrqapgkerefvaaitPYSFKtyyadsvkgrftisrddarntvylqmnslkpedtavyycar
TYYSTTCLLFdywgqgtqvtvss#ATATACCATGGAAGTTCAACTGGTTGAATCTGGTGGCGGTTTAGTTCAAGCCGGTGATAGCCTTCGTCTGAGCTGCGCCGCA
AGCGGAGATTACTGCATGGATTGCTTTATGGGTTGGTTTCGCCAGGCACCTGGTAAAGAACGTGAATTTGTGGCAGCCATTACCCCCTATTCCTTTAAGACCT
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTGCGCGTACGTACTACAGCACCACCTGTTTGCTGTTCGACTATTGGGGTCAGGGCACACAAGTCACGGTCTCAAGCGGCGGAAGCTCTGGTGA
ACAGAAACTGATTTCAGAAG
DYCMDCF#PYSFK#PLNIGTVTIA#evqlvesggglvqagdslrlscaasgDYCMDCFmgwfrqapgkerefvaaitPYSFKtyyadsvkgrftisrddarntvylqmnslkpedtavyycaa
PLNIGTVTIAdywgqgtqvtvss#GAAGGAGATATACCATGGAAGTTCAACTGGTTGAATCTGGTGGCGGTTTAGTTCAAGCCGGTGATAGCCTTCGTCTGAGCT
GCGCCGCAAGCGGAGATTACTGCATGGATTGCTTTATGGGTTGGTTTCGCCAGGCACCTGGTAAAGAACGTGAATTTGTGGCAGCCATTACCCCCTATTCCT
TTAAGACCTACTATGCCGATTCTGTTAAAGGTCGCTTCACTATTTCACGCGATGATGCGCGTAATACCGTGTACTTACAGATGAACTCACTGAAACCTGAGGA
TACCGCCGTTTATTATTGTGCGGCACCCCTCAATATCGGCACCGTGACGATTGCCGACTATTGGGGTCAGGGCACACAAGTCACGGTCTCAAGCGGCGGAA
GCTCTGGTGAACAGAAACTGATTTCAGAAG
DYCMDCF#PYSFK#QVSLVTHGDL#evqlvesggglvqagdslrlscaasgDYCMDCFmgwfrqapgkerefvaaitPYSFKtyyadsvkgrftisrddarntvylqmnslkpedtavyyc
aaQVSLVTHGDLdswgqgtqvtvss#ATACCATGGAAGTTCAACTGGTTGAATCTGGTGGCGGTTTAGTTCAAGCCGGTGATAGCCTTCGTCTGAGCTGCGCCGC
AAGCGGAGATTACTGCATGGATTGCTTTATGGGTTGGTTTCGCCAGGCACCTGGTAAAGAACGTGAATTTGTGGCAGCCATTACCCCCTATTCCTTTAAGAC
CTACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGCACAGGTCTCCCTTGTTACCCATGGGGATCTGGACTCTTGGGGTCAGGGCACACAAGTCACGGTCTCAAGCGGCGGAAGCTCTGG
TGAACAGAAACTGATTTCAGAAG
DYCMDCF#PYSFK#NPLDPNWTNS#evqlvesggglvqagdslhlscaasgDYCMDCFmgwfrqapgkerefvaaitPYSFKtyyadsvkgrftisrddarntvylqmnslkpedtavyy
caaNPLDPNWTNSdywgqgtqvtvss#ATATACCATGGAAGTTCAACTGGTTGAATCTGGTGGCGGTTTAGTTCAAGCCGGTGATAGCCTTCATCTGAGCTGCG
CCGCAAGCGGAGATTACTGCATGGATTGCTTTATGGGTTGGTTTCGCCAGGCACCTGGTAAAGAACGTGAATTTGTGGCAGCCATTACCCCCTATTCCTTTA
AGACCTACTATGCCGATTCTGTTAAAGGTCGCTTCACTATTTCACGCGATGATGCGCGTAATACCGTGTACTTACAGATGAACTCACTGAAACCTGAGGATAC
CGCCGTTTATTATTGTGCGGCAAACCCTCTGGACCCCAACTGGACTAATTCTGACTATTGGGGTCAGGGCACACAAGTCACGGTCTCAAGCGGCGGAAGCTC
TGGTGAACAGAAACTGATTTCAGAAG
DYCMDCF#PYSFK#SANILHLSVI#evqlvesggglvkagdslrlscaasgDYCMDCFmgwfrqapgkerefvaaitPYSFKtyyadsvkgrftisrddarntvylqmnslkpedtavyycaa
SANILHLSVInywgqgtqvtvss#AGGAGATATACCATGGAAGTTCAACTGGTTGAATCTGGTGGCGGTTTAGTTAAAGCCGGTGATAGCCTTCGTCTGAGCTGC
GCCGCAAGCGGAGATTACTGCATGGATTGCTTTATGGGTTGGTTTCGCCAGGCACCTGGTAAAGAACGTGAATTTGTGGCAGCCATTACCCCCTATTCCTTT
AAGACCTACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGGCAAGCGCGAACATTCTCCATTTGTCTGTTATCAACTATTGGGGTCAGGGCACACAAGTCACGGTCTCAAGCGGCGGAAGCT
CTGGTGAACAGAAACTGATTTCAGAAG
DYCMDCF#PYSFK#SSCLGALLHL#evqlvesggglvqasdslrlscaasgDYCMDCFmgwfrqapgkerefvaaitPYSFKtyyadsvkgrftisrddarntvylqmnslkpedtavyycaa
SSCLGALLHLdywgqgtqvtvss#ATATACCATGGAAGTTCAACTGGTTGAATCTGGTGGCGGTTTAGTTCAAGCCAGTGATAGCCTTCGTCTGAGCTGCGCCGC
AAGCGGAGATTACTGCATGGATTGCTTTATGGGTTGGTTTCGTCAGGCACCTGGTAAAGAACGTGAATTTGTGGCAGCCATTACCCCCTATTCCTTTAAGAC
CTACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGCATCGTCTTGCTTGGGTGCTCTCCTTCATTTGGACTATTGGGGTCAGGGCACACAAGTCACGGTCTCAAGCGGCGGAAGCTCTGGT
GAACAGAAACTGATTTCAGAAG
DYCMDCF#PYSFK#CLPNAH#evqlvesggglvqagdslrlscaasgDYCMDCFmgwfrqapgkerefvaaitPYSFKtyyadsvkgrftisrddarntvylqmnslkpedtavyycaaCLP
NAHdywgqgtqvtvss#ATATGCCATGGAAGTTCAACTGGTTGAATCTGGTGGCGGTTTAGTTCAAGCCGGTGATAGCCTTCGTCTGAGCTGCGCCGCAAGCG
GAGATTACTGCATGGATTGCTTTATGGGTTGGTTTCGCCAGGCACCTGGTAAAGAACGTGAATTTGTGGCAGCCATTACCCCCTATTCCTTTAAGACCTACTA
TGCCGATTCTGTTAAAGGTCGCTTCACTATTTCACGCGATGATGCGCGTAATACCGTGTACTTACAGATGAACTCACTGAAACCTGAGGATACCGCCGTTTAT
TATTGTGCGGCATGCCTCCCCAATGCTCATGACTATTGGGGTCAGGGCACACAAGTCACGGTCTCAAGCGGCGGAAGCTCTGGTGAACAGAAACTGATTTC
AGAAG
DYCMDCF#PYSFK#QIRKFSVFW#evqlvesggglvqagdslrlscaasgDYCMDCFmgwfrqapgkerefvaaitPYSFKtyyadsvkgrftisrddarntvylqmnslkpedtavyycaa
QIRKFSVFWdywgqgtqvtvss#ATATACCATGGAAGTTCAACTGGTTGAATCTGGTGGCGGTTTAGTTCAAGCCGGTGATAGCCTTCGTCTGAGCTGCGCCGC
AAGCGGAGATTACTGCATGGATTGCTTTATGGGTTGGTTTCGCCAGGCACCTGGTAAAGAACGTGAATTTGTGGCAGCCATTACCCCCTATTCCTTTAAGAC
CTACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGCACAGATTCGCAAGTTTAGTGTGTTTTGGGACTATTGGGGTCAGGGCACACAAGTCACGGTCTCAAGCGGCGGAAGCTCTGGTGA
ACAGAAACTGATTTCAGAAG
DYCMDCF#PYSFK#QVSLVTHGDL#evqlvksggglvqagdslrlscaasgDYCMDCFmgwfrqapgkerefvaaitPYSFKtyyadsvkgrftisrddarntvylqmnslkpedtavyyca
aQVSLVTHGDLdswgqgtqvtvss#AGATATACCATGGAAGTTCAACTGGTTAAATCTGGTGGCGGTTTAGTTCAAGCCGGTGATAGCCTTCGTCTGAGCTGCGC
CGCAAGCGGAGATTACTGCATGGATTGCTTTATGGGTTGGTTTCGCCAGGCACCTGGTAAAGAACGTGAATTTGTGGCAGCCATTACCCCCTATTCCTTTAA
GACCTACTATGCCGATTCTGTTAAAGGTCGCTTCACTATTTCACGCGATGATGCGCGTAATACCGTGTACTTACAGATGAACTCACTGAAACCTGAGGATACC
GCCGTTTATTATTGTGCGGCACAGGTCTCCCTTGTTACCCATGGGGATCTGGACTCTTGGGGTCAGGGCACACAAGTCACGGTCTCAAGCGGCGGAAGCTCT
GGTGAACAGAAACTGATTTCAGAAG
DYCMDCF#PYSFK#NPLDPNWTNS#evqlvesggglvhagdslrlscaasgDYCMDCFmgwfrqapgkerefvaaitPYSFKtyyadsvkgrftisrddarntvylqmnslkpedtavyyc
aaNPLDPNWTNSdywgqgtqvtvss#GAAGGAGATATACCATGGAAGTTCAACTGGTTGAATCTGGTGGCGGTTTAGTTCACGCCGGTGATAGCCTTCGTCTGA
GCTGCGCCGCAAGCGGAGATTACTGCATGGATTGCTTTATGGGTTGGTTTCGCCAGGCACCTGGTAAAGAACGTGAATTTGTGGCAGCCATTACCCCCTATT
CCTTTAAGACCTACTATGCCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGGCAAACCCTCTGGACCCCAACTGGACTAATTCTGACTATTGGGGTCAGGGCACACAAGTCACGGTCTCAAGCGGCG
GAAGCTCTGGTGAACAGAAACTGATTTCAGAAG
DYCMDCF#PYSFK#CRTTTCYGF#evqlvesggglvqagdslrlscaasgDYCMDCFmgwfrqapgkerefvaaitPYSFKtyyadsvkgrftisrddarntvylqmnslkpedtavyycat
CRTTTCYGFdywgqgtqvtvss#ATATACCATGGAAGTTCAACTGGTTGAATCTGGTGGCGGTTTAGTTCAAGCCGGTGATAGCCTTCGTCTGAGCTGCGCCGCA
AGCGGAGATTACTGCATGGATTGCTTTATGGGTTGGTTTCGCCAGGCACCTGGTAAAGAACGTGAATTTGTGGCAGCCATTACCCCCTATTCCTTTAAGACCT
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTGCGACATGTAGGACCACTACGTGTTATGGTTTTGACTATTGGGGTCAGGGCACACAAGTCACGGTCTCAAGCGGCGGAAGCTCTGGTGAAC
AGAAACTGATTTCAGAAG
DYCMDCF#PYSFK#NPLDPNWTNS#evqlvesggvlvqagdslrlscaasgDYCMDCFmgwfrqapgkerefvaaitPYSFKtyyadsvkgrftisrddarntvylqmnslkpedtavyyc
aaNPLDPNWTNSdywgqgtqvtvss#AGGAGATATACCATGGAAGTTCAACTGGTTGAATCTGGTGGCGTTTTAGTTCAAGCCGGTGATAGCCTTCGTCTGAGC
TGCGCCGCAAGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTTCACTATTTCACGCGATGATGCGCGTAATACCGTGTACTTACAGATGAACTCACTGAAACCTGAGG
ATACCGCCGTTTATTATTGTGCGGCAAACCCTCTGGACCCCAACTGGACTAATTCTGACTATTGGGGTCAGGGCACACAAGTCACGGTCTCAAGCGGCGGAA
GCTCTGGTGAACAGAAACTGATTTCAGAAG
DYCMDCF#PYSFK#WLTTQPFCGW#evqlvesggglvqagdslrlscaasgDYCMDCFmgwfrqapgkerefvaaitPYSFKtyyadsvrgrfsisadsakntvylqmnslkpedtavyy
caaWLTTQPFCGWnywgqgtqvtvss#ACCATGGAAGTTCAACTGGTTGAATCTGGTGGCGGTTTAGTTCAAGCCGGTGATAGCCTTCGTCTGAGCTGCGCCGC
AAGCGGAGATTACTGCATGGATTGCTTTATGGGTTGGTTTCGCCAGGCACCTGGTAAAGAACGTGAATTTGTGGCAGCCATTACCCCCTATTCCTTTAAGAC
CTACTATGCCGATTCTGTTCGTGGTCGCTTCAGCATTTCAGCAGATTCTGCGAAGAATACCGTGTACTTACAGATGAACTCACTGAAACCTGAGGATACCGCC
GTTTATTATTGTGCGGCATGGCTGACCACTCAGCCGTTCTGCGGTTGGAACTATTGGGGTCAGGGCACACAAGTCACGGTCTCAAGCGGCGGAAGCTCTGG
TGAACAGAAACTGATTTCAGAAG
DYCMDCF#PYSFK#GICAPSNTLGFVL#evqlvesggglvqagdslrlscaasgDYCMDCFmgwfrqapgkerefvaaitPYSFKtyyadsvkgrftisrddarntvylqmnslkpedtavy
ycaaGICAPSNTLGFVLgywgqgtqvtvss#GAAGGAGATATACCATGGAAGTTCAACTGGTTGAATCTGGTGGCGGTTTAGTTCAAGCCGGTGATAGCCTTCGTC
TGAGCTGCGCCGCAAGCGGAGATTACTGCATGGATTGCTTTATGGGTTGGTTTCGCCAGGCACCTGGTAAAGAACGTGAATTTGTGGCAGCCATTACCCCCT
ATTCCTTTAAGACCTACTATGCCGATTCTGTTAAAGGTCGCTTCACTATTTCACGCGATGATGCGCGTAATACCGTGTACTTACAGATGAACTCACTGAAACCT
GAGGATACCGCCGTTTATTATTGTGCGGCAGGGATCTGTGCCCCCTCCAATACGCTCGGCTTCGTTCTTGGCTATTGGGGTCAGGGCACACAAGTCACGGTC
TCAAGCGGCGGAAGCTCTGGTGAACAGAAACTGATTTCAGAAG
DYCMDCF#PYSFK#DSLILANSQW#evqlvesggglvhagdrlrlscaasgDYCMDCFmgwfrqapgkerefvaaitPYSFKtyyadsvkgrftisrddarntvylqmnslkpedtavyyca
aDSLILANSQWdywgqgtqvtvss#GAAGGAGATATACCATGGAAGTTCAACTGGTTGAATCTGGTGGCGGTTTAGTTCACGCCGGTGATCGCCTTCGTCTGAG
CTGCGCCGCAAGCGGAGATTACTGCATGGATTGCTTTATGGGTTGGTTTCGCCAGGCACCTGGTAAAGAACGTGAATTTGTGGCAGCCATTACCCCCTATTC
CTTTAAGACCTACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTGCGGCAGACAGCCTTATTTTGGCTAATTCTCAGTGGGACTATTGGGGTCAGGGCACACAAGTCACGGTCTCAAGCGGCGGA
AGCTCTGGTGAACAGAAACTGATTTCAGAAG
DYCMDCF#PYSFK#PFAPLTVLLA#evqlvesggglvqagdslrlscaasgDYCMDCFmgwfrqapgkerefvaaitPYSFKtyyadsvkgrftisrddarntvylqmnslkpedtavyyca
aPFAPLTVLLAeywgqdtqvtvss#ACCATGGAAGTTCAACTGGTTGAATCTGGTGGCGGTTTAGTTCAAGCCGGTGATAGCCTTCGTCTGAGCTGCGCCGCAAG
CGGAGATTACTGCATGGATTGCTTTATGGGTTGGTTTCGCCAGGCACCTGGTAAAGAACGTGAATTTGTGGCAGCCATTACCCCCTATTCCTTTAAGACCTAC
TATGCCGATTCTGTTAAAGGTCGCTTCACTATTTCACGCGATGATGCGCGTAATACCGTGTACTTACAGATGAACTCACTGAAACCTGAGGATACCGCCGTTT
ATTATTGTGCGGCACCGTTCGCGCCCCTTACCGTTCTTCTCGCGGAGTATTGGGGTCAGGACACACAAGTCACGGTCTCAAGCGGCGGAAGCTCTGGTGAAC
AGAAACTGATTTCAGAAG
DYCMDCF#PYSFR#DSLILANSQW#evqlvesggglvqagdslrlscaasgDYCMDCFmgwfrqapgkerefvaaitPYSFRtyyadsvkgrftisrddarntvylqmnslkpedtavyyc
aaDSLILANSQWdywgqgtqvtvss#ACCATGGAAGTTCAACTGGTTGAATCTGGTGGCGGTTTAGTTCAAGCCGGTGATAGCCTTCGTCTGAGCTGCGCCGCA
AGCGGAGATTACTGCATGGATTGCTTTATGGGTTGGTTTCGCCAGGCACCTGGTAAAGAACGTGAATTTGTGGCAGCCATTACCCCCTATTCCTTTAGGACC
TACTATGCCGATTCTGTTAAAGGTCGCTTCACTATTTCACGCGATGATGCGCGTAATACCGTGTACTTACAGATGAACTCACTGAAACCTGAGGATACCGCCG
TTTATTATTGTGCGGCAGACAGCCTTATTTTGGCTAATTCTCAGTGGGACTATTGGGGTCAGGGCACACAAGTCACGGTCTCAAGCGGCGGAAGCTCTGGTG
AACAGAAACTGCTTTCAGAAG
DYCMDCF#PYSFK#VPNGTWSERA#evqlvesggglvqagdslrlscaasgDYCMDCFmgwfrqapgkerefvaaitPYSFKtyyadsvkgrftisrddarntvylqmnslkpedtavyyc
aaVPNGTWSERAdywgqgtqvtvss#AGATATACCATGGAAGTTCAACTGGTTGAATCTGGTGGCGGTTTAGTTCAAGCCGGTGATAGCCTTCGTCTGAGCTGC
GCCGCAAGCGGAGATTACTGCATGGATTGCTTTATGGGTTGGTTTCGCCAGGCACCTGGTAAAGAACGTGAATTTGTGGCAGCCATTACCCCCTATTCCTTT
AAGACCTACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGGCAGTCCCCAATGGGACCTGGTCCGAGAGGGCTGACTATTGGGGTCAGGGCACACAAGTCACGGTCTCAAGCGGCGGAAG
CTCTGGTGAACAGAAACTGATTTCAGAAG
DYCMDCF#PYSFK#NPLDPNWTNS#evqlvesggglvqagdslrlscaaggDYCMDCFmgwfrqapgkerefvaaitPYSFKtyyadsvkgrftisrddarntvylqmnslkpedtavyy
caaNPLDPNWTNSaywgqgtqvtvss#ACCATGGAAGTTCAACTGGTTGAATCTGGTGGCGGTTTAGTTCAAGCCGGTGATAGCCTTCGTCTGAGCTGCGCCGC
AGGCGGAGATTACTGCATGGATTGCTTTATGGGTTGGTTTCGCCAGGCACCTGGTAAAGAACGTGAATTTGTGGCAGCCATTACCCCCTATTCCTTTAAGAC
CTACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGCAAACCCTCTGGACCCCAACTGGACTAATTCTGCCTATTGGGGTCAGGGCACACAAGTCACGGTCTCAAGCGGCGGAAGCTCTGGT
GAACAGAAACTGATTTCAGAAG
DYCMDCF#PYSFK#NPLDPSWTNS#evqlvesggglvqagdslrlscaasgDYCMDCFmgwfrqapgkerefvaaitPYSFKtyyadsvkgrftisrddarntvylqmnslkpedtavyyc
aaNPLDPSWTNSdywgqgtqvtvss#AAGGAGATATACCATGGAAGTTCAACTGGTTGAATCTGGTGGCGGTTTAGTTCAAGCCGGTGATAGCCTTCGTCTGAG
CTGCGCCGCAAGCGGAGATTACTGCATGGATTGCTTTATGGGTTGGTTTCGCCAGGCACCTGGTAAAGAACGTGAATTTGTGGCAGCCATTACCCCCTATTC
CTTTAAGACCTACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTGCGGCAAACCCTCTGGACCCCAGCTGGACTAATTCTGACTATTGGGGTCAGGGCACACAAGTCACGGTCTCAAGCGGCGG
AAGCTCTGGTGAACAGAAACTGATTTCAGAAG
DYCMDCF#PYSFK#VPNVTWSERA#evqlvesggglvqagdslrlscaasgDYCMDCFmgwfrqapgkerefvaaitPYSFKtyyadsvkgrftisrdaarntvylqmnslkpedtavyyc
aaVPNVTWSERAdywgqgtqvtvss#ATACCATGGAAGTTCAACTGGTTGAATCTGGTGGCGGTTTAGTTCAAGCCGGTGATAGCCTTCGTCTGAGCTGCGCCG
CAAGCGGAGATTACTGCATGGATTGCTTTATGGGTTGGTTTCGCCAGGCACCTGGTAAAGAACGTGAATTTGTGGCAGCCATTACCCCCTATTCCTTTAAGA
CCTACTATGCCGATTCTGTTAAAGGTCGCTTCACTATTTCACGCGATGCTGCGCGTAATACCGTGTACTTACAGATGAACTCACTGAAACCTGAGGATACCGC
CGTTTATTATTGTGCGGCAGTCCCCAATGTGACCTGGTCCGAGAGGGCTGACTATTGGGGTCAGGGCACACAAGTCACGGTCTCAAGCGGCGGAAGCTCTG
GTGAACAGAAACTGATTTCAGAAG
DYCMDCF#PYSFK#PLNIGTATIA#evqlvesggglvhagdrlrlscaasgDYCMDCFmgwfrqapgkerefvaaitPYSFKtyyadsvkgrftisrddarntvylqmnslkpedtavyycaa
PLNIGTATIAdywgqgtqvtvss#GAAGGAGATATACCATGGAAGTTCAACTGGTTGAATCTGGTGGCGGTTTAGTTCACGCCGGTGATCGCCTTCGTCTGAGCT
GCGCCGCAAGCGGAGATTACTGCATGGATTGCTTTATGGGTTGGTTTCGCCAGGCACCTGGTAAAGAACGTGAATTTGTGGCAGCCATTACCCCCTATTCCT
TTAAGACCTACTATGCCGATTCTGTTAAAGGTCGCTTCACTATTTCACGCGATGATGCGCGTAATACCGTGTACTTACAGATGAACTCACTGAAACCTGAGGA
TACCGCCGTTTATTATTGTGCGGCACCCCTCAATATCGGCACCGCGACGATTGCCGACTATTGGGGTCAGGGCACACAAGTCACGGTCTCAAGCGGCGGAA
GCTCTGGTGAACAGAAACTGATTTCAGAAG
DYCMDCF#PYSFK#PFAPLTVLLA#evqlvesggglvqagdslrlscaasgDYCMDCFmgwfrqapgkerefvaaitPYSFKtyyadsvkgrftisrddarntvylqmnslkpedtavyyca
aPFAPLTVLLAeywgqgtqvtvss#AGGAGATATACCATGGAAGTTCAACTGGTTGAATCTGGTGGCGGTTTAGTTCAAGCCGGTGATAGCCTTCGTCTGAGCTG
CGCCGCAAGCGGAGATTACTGCATGGATTGCTTTATGGGTTGGTTTCGCCAGGCACCTGGTAAAGAACGTGAATTTGTGGCAGCCATTACCCCCTATTCCTT
TAAGACCTACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTGCGGCACCGTTCGCGCCCCTTACCGTTCTTCTCGCGGAGTATTGGGGTCAGGGCACACAAGTCACGGTCTCAAGCGGCGGAAGC
TCTGGTGAACAGAAACTGATTTCAGAAG
DYCMDCF#PYSFK#VPNVTWSERA#evqlvesggglvqagdslrlscaasgDYCMDCFmgwfrqapgkerefvaaitPYSFKtyyadsvkgrftisrddarntvylqmnslkpedtavyyc
aaVPNVTWSERAdywgqgtqvtvss#AGGAGATATACCATGGAAGTTCAACTGGTTGAATCTGGTGGCGGTTTAGTTCAAGCCGGTGATAGCCTTCGTCTGAGC
TGCGCCGCAAGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTTCACTATTTCACGCGATGATGCGCGTAATACCGTGTACTTACAGATGAACTCACTGAAACCTGAGG
ATACCGCCGTTTATTATTGTGCGGCAGTCCCCAATGTGACCTGGTCCGAGAGGGCTGACTATTGGGGTCAGGGCACACAAGTCACGGTCTCAAGCGGCGGA
AGCTCTGGTGAACAGAAACTGATTTCAGAAG
DYCMDCF#PYSFK#NPLDPNWTNS#evqlvesggglvqagdslrpscaasgDYCMDCFmgwfrqapgkerefvaaitPYSFKtyyadsvkgrftisrddarntvylqmnslkpedtavyy
caaNPLDPNWTNSdywgqgtqvtvss#AGGAGATATACCATGGAAGTTCAACTGGTTGAATCTGGTGGCGGTTTAGTTCAAGCCGGTGATAGCCTTCGTCCGAG
CTGCGCCGCAAGCGGAGATTACTGCATGGATTGCTTTATGGGTTGGTTTCGCCAGGCACCTGGTAAAGAACGTGAATTTGTGGCAGCCATTACCCCCTATTC
CTTTAAGACCTACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTGCGGCAAACCCTCTGGACCCCAACTGGACTAATTCTGACTATTGGGGTCAGGGCACACAAGTCACGGTCTCAAGCGGCGGA
AGCTCTGGTGAACAGAAACTGATTTCAGAAG
DYCMDCF#PYSFK#PLNIGTATIA#evqlvesggglvqagdslrlscaasgDYCMDCFmgwfrqapgkerefvaaitPYSFKtyyadsvkgrftisrddarntvylqmnslkpedtavyycat
PLNIGTATIAdywgqgtqvtvss#ATACCATGGAAGTTCAACTGGTTGAATCTGGTGGCGGTTTAGTTCAAGCCGGTGATAGCCTTCGTCTGAGCTGCGCCGCAA
GCGGAGATTACTGCATGGATTGCTTTATGGGTTGGTTTCGCCAGGCACCTGGTAAAGAACGTGAATTTGTGGCAGCCATTACCCCCTATTCCTTTAAGACCTA
CTATGCCGATTCTGTTAAAGGTCGCTTCACTATTTCACGCGATGATGCGCGTAATACCGTGTACTTACAGATGAACTCACTGAAACCTGAGGATACCGCCGTT
TATTATTGTGCGACACCCCTCAATATCGGCACCGCGACGATTGCCGACTATTGGGGTCAGGGCACACAAGTCACGGTCTCAAGCGGCGGAAGCTCTGGTGA
ACAGAAACTGATTTCAGAAG
DYCMDFF#PYSFK#WLTTQPFYGW#evqlvesggglvqagdrIrIncaasgDYCMDFFmgwfrqapgkerefvaaitPYSFKtyyadsvrgrfsisadsakntvylqmnslkpedtavyy
caaWLTTQPFYGWnywgqgtqvtvss#GGAGATATACCATGGAAGTTCAACTGGTTGAATCTGGTGGCGGTTTAGTTCAAGCCGGTGATAGGCTTCGTCTGAAC
TGCGCCGCAAGCGGAGATTACTGCATGGATTTCTTTATGGGTTGGTTTCGCCAAGCACCTGGTAAAGAACGTGAATTTGTGGCCGCCATTACCCCCTATTCCT
TTAAGACCTACTATGCCGATTCTGTTCGTGGTCGCTTCAGCATTTCAGCAGATTCTGCGAAGAATACCGTGTACTTACAGATGAACTCACTGAAACCTGAGGA
TACCGCCGTTTATTATTGTGCGGCATGGCTGACCACTCAGCCGTTCTACGGTTGGAACTATTGGGGTCAGGGCACACAAGTCACGGTCTCAAGCGGCGGAA
GCTCTTGTGAACAGAAACTGATTTCAGAAG
DYCMDCF#PYSFK#TLNIGTATIA#evqlvesggglvqagdslrlscaasgDYCMDCFmgwfrqapgkerefvaaitPYSFKtyyadsvkgrftisrddarntvylqmtslkpedtavyycaa
TLNIGTATIAdywgqgtqvtvss#ACCATGGAAGTTCAACTGGTTGAATCTGGTGGCGGTTTAGTTCAAGCCGGTGATAGCCTTCGTCTGAGCTGCGCCGCAAGC
GGAGATTACTGCATGGATTGCTTTATGGGTTGGTTTCGCCAGGCACCTGGTAAAGAACGTGAATTTGTGGCAGCCATTACCCCCTATTCCTTTAAGACCTACT
ATGCCGATTCTGTTAAAGGTCGCTTCACTATTTCACGCGATGATGCGCGTAATACCGTGTACTTACAGATGACCTCCCTGAAACCTGAGGATACCGCCGTTTA
TTATTGTGCGGCAACCCTCAATATCGGCACCGCGACGATTGCCGACTATTGGGGTCAGGGCACACAAGTCACGGTCTCAAGCGGCGTAAGCTCTGGTGAAC
AGAAACTGATTTCAGAAG
DYCMDCF#PYSFK#VPNVTWSERA#evqlvesggglvqagdsfrlscaasgDYCMDCFmgwfrqapgkerefvaaitPYSFKtyyadsvkgrftisrddarntvylqmnslkpedtavyyc
aaVPNVTWSERAdywgqgtqvtvss#AAGGAGATATACCATGGAAGTTCAACTGGTTGAATCTGGTGGCGGTTTAGTTCAAGCCGGTGATAGCTTTCGTCTGAG
CTGCGCCGCAAGCGGAGATTACTGCATGGATTGCTTTATGGGTTGGTTTCGCCAGGCACCTGGTAAAGAACGTGAATTTGTGGCAGCCATTACCCCCTATTC
CTTTAAGACCTACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTGCGGCAGTCCCCAATGTGACCTGGTCCGAGAGGGCTGACTATTGGGGTCAGGGCACACAAGTCACGGTCTCAAGCGGCGG
AAGCTCTGGTGAACAGAAACTGATTTCAGAAG
DYCMDCF#PYSFK#PLNIGTATIA#evqlvesggglvqagdslrlscaasgDYCMDCFmgwfrqapgkerefvaaitPYSFKtyyadsvkgrftisrddarntmylqmnslkpedtavyyca
aPLNIGTATIAdywgqgtqvtvss#AGATATACCATGGAAGTTCAACTGGTTGAATCTGGTGGCGGTTTAGTTCAAGCCGGTGATAGCCTTCGTCTGAGCTGCGC
CGCAAGCGGAGATTACTGCATGGATTGCTTTATGGGTTGGTTTCGCCAGGCACCTGGTAAAGAACGTGAATTTGTGGCAGCCATTACCCCCTATTCCTTTAA
GACCTACTATGCCGATTCTGTTAAAGGTCGCTTCACTATTTCACGCGATGATGCGCGTAATACCATGTACTTACAGATGAACTCACTGAAACCTGAGGATACC
GCCGTTTATTATTGTGCGGCACCCCTCAATATCGGCACCGCGACGATTGCCGACTATTGGGGTCAGGGCACACAAGTCACGGTCTCAAGCGGCGGAAGCTC
TGGTGAACAGAAACTGATTTCAGAAG
DYCMDCF#PYSFK#NPLDPNWTNS#evqlvesggglvqagdslrlscaaggDYCMDCFmgwfrqapgkerefvaaitPYSFKtyyvdsvkgrftisrddarntvylqmnslkpedtavyyc
aaNPLDPNWTNSdywgqgtqvtvss#AAGGAGATATACCATGGAAGTTCAACTGGTTGAATCTGGTGGCGGTTTAGTTCAAGCCGGTGATAGCCTTCGTCTGA
GCTGCGCCGCAGGCGGAGATTACTGCATGGATTGCTTTATGGGTTGGTTTCGCCAGGCACCTGGTAAAGAACGTGAATTTGTGGCAGCCATTACCCCCTATT
CCTTTAAGACCTACTATGTCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGGCAAACCCTCTGGACCCCAACTGGACTAATTCTGACTATTGGGGTCAGGGCACACAAGTCACGGTCTCAAGCGGCG
GAAGCTCTGGTGAACAGAAACTGATTTCAGAAG
DYCMDCF#PYSFK#DICAGS#evqlvesggglvqagdslrlscaasgDYCMDCFmgwfrqapgkerefvaaitPYSFKtyyadsvkgrftisrddarntvylqmnslkpedtavyycaaDIC
AGSdswgqgtqvtvss#ATACCATGGAAGTTCAACTGGTTGAATCTGGTGGCGGTTTAGTTCAAGCCGGTGATAGCCTTCGTCTGAGCTGCGCCGCAAGCGGAG
ATTACTGCATGGATTGCTTTATGGGTTGGTTTCGCCAGGCACCTGGTAAAGAACGTGAATTTGTGGCAGCCATTACCCCCTATTCCTTTAAGACCTACTATGC
CGATTCTGTTAAAGGTCGCTTCACTATTTCACGCGACGATGCGCGTAATACCGTGTACTTACAGATGAACTCACTGAAACCTGAGGATACCGCCGTTTATTAT
TGTGCGGCAGACATTTGTGCTGGCAGCGACTCTTGGGGTCAGGGCACACAAGTCACGGTCTCAAGCGGCGGAAGCTCTGGTGAACAGAAACTGATTTCAG
AAG
DYCMDCF#PYSFK#NPLDPNWTNS#evqlvesggglvhagdrlrlscaaggDYCMDCFmgwfrqapgkerefvaaitPYSFKtyyadsvkgrftisrddarntvylqmnslkpedtavyyc
aaNPLDPNWTNSdywgqgtqvtvss#GAAGGAGATATACCATGGAAGTTCAACTGGTTGAATCTGGTGGCGGTTTAGTTCACGCCGGTGATCGCCTTCGTCTGA
GCTGCGCCGCAGGCGGAGATTACTGCATGGATTGCTTTATGGGTTGGTTTCGCCAGGCACCTGGTAAAGAACGTGAATTTGTGGCAGCCATTACCCCCTATT
CCTTTAAGACCTACTATGCCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGGCAAACCCTCTGGACCCCAACTGGACTAATTCTGACTATTGGGGTCAGGGCACACAAGTCACGGTCTCAAGCGGCG
GAAGCTCTGGTGAACAGAAACTGATTTCAGAAG
DYCMDCF#PYSFK#PLNIGTATIA#evqlvesggglvkagdslrlscaasgDYCMDCFmgwfrqapgkerefvaaitPYSFKtyyadsvkgrftisrddarntvylqmnslkpedtavyycaa
PLNIGTATIAdywgqgtqvtvss#AGGAGATATACCATGGAAGTTCAACTGGTTGAATCTGGTGGCGGTTTAGTTAAAGCCGGTGATAGCCTTCGTCTGAGCTGC
GCCGCAAGCGGAGATTACTGCATGGATTGCTTTATGGGTTGGTTTCGCCAGGCACCTGGTAAAGAACGTGAATTTGTGGCAGCCATTACCCCCTATTCCTTT
AAGACCTACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGGCACCCCTCAATATCGGCACCGCGACGATTGCCGACTATTGGGGTCAGGGCACACAAGTCACGGTCTCAAGCGGCGGAAGC
TCTGGTGAACAGAAACTGATTTCAGAAG
DYCMDCF#PYSFK#PVISFAADGW#evqlvesggglvqagdslrlscaasgDYCMDCFmgwfrqapgkerefvaaitPYSFKtyyadsvkgrftisrddarntvylqmnslkpedtavyyc
aaPVISFAADGWaywgqgtqvtvss#AGGAGATATACCATGGAAGTTCAACTGGTTGAATCTGGTGGCGGTTTAGTTCAAGCCGGTGATAGCCTTCGTCTGAGC
TGCGCCGCAAGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTTCACTATTTCACGCGATGATGCGCGTAATACCGTGTACTTACAGATGAACTCACTGAAACCTGAGG
ATACCGCCGTTTATTATTGTGCGGCACCCGTCATCTCCTTTGCTGCGGACGGCTGGGCCTATTGGGGTCAGGGCACACAAGTCACGGTTTCAAGCGGCGGAA
GCTCTGGTGAACAGAAACTGATTTCAGAAG
DYCMDCF#PYSFK#DSLILANSQW#evqlfesggglvqagdslrlscaasgDYCMDCFmgwfrqapgkerefvaaitPYSFKtyyadsvkgrftisrddarntvylqmnslkpedtavyyca
aDSLILANSQWdywgqgtqvtvss#ATATACCATGGAAGTTCAACTGTTTGAATCTGGTGGCGGTTTAGTTCAAGCCGGTGATAGCCTTCGTCTGAGCTGCGCCG
CAAGCGGAGATTACTGCATGGATTGCTTTATGGGTTGGTTTCGCCAGGCACCTGGTAAAGAACGTGAATTTGTGGCAGCCATTACCCCCTATTCCTTTAAGA
CCTACTATGCCGATTCTGTTAAAGGTCGCTTCACTATTTCACGCGATGATGCGCGTAATACCGTGTACTTACAGATGAACTCACTGAAACCTGAGGATACCGC
CGTTTATTATTGTGCGGCAGACAGCCTTATTTTGGCTAATTCTCAGTGGGACTATTGGGGTCAGGGCACACAAGTCACGGTCTCAAGCGGCGGAAGCTCTGG
TGAACAGAAACTGATTTCAGAAG
DYCMDCF#PYSFK#NPLDPNWTNS#evqlvdsggglvqagdslrlscaasgDYCMDCFmgwfrqapgkerefvaaitPYSFKtyyadsvkgrftisrddarntvylqmnslkpedtavyyc
aaNPLDPNWTNSdywgqgtqvtvss#GAAGGAGATATACCATGGAAGTTCAACTGGTTGACTCTGGTGGCGGTTTAGTTCAAGCCGGTGATAGCCTTCGTCTGA
GCTGCGCCGCAAGCGGAGATTACTGCATGGATTGCTTTATGGGTTGGTTTCGCCAGGCACCTGGTAAAGAACGTGAATTTGTGGCAGCCATTACCCCCTATT
CCTTTAAGACCTACTATGCCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGGCAAACCCTCTGGACCCCAACTGGACTAATTCTGACTATTGGGGTCAGGGCACACAAGTCACGGTCTCAAGCGGCG
GAAGCTCTGGTGAACAGAAACTGATTTCAGAAG
DYCMDCF#PYSFK#NPLDPNWTNS#evqlfesggglvqagdslrlscaasgDYCMDCFmgwfrqapgkerefvaaitPYSFKtyyadsvkgrftisrddarntvylqmnslkpedtavyyc
aaNPLDPNWTNSdywgqgtqvtvss#AGATATACCATGGAAGTTCAACTGTTTGAATCTGGTGGCGGTTTAGTTCAAGCCGGTGATAGCCTTCGTCTGAGCTGC
GCCGCAAGCGGAGATTACTGCATGGATTGCTTTATGGGTTGGTTTCGCCAGGCACCTGGTAAAGAACGTGAATTTGTGGCAGCCATTACCCCCTATTCCTTT
AAGACCTACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGGCAAACCCTCTGGACCCCAACTGGACTAATTCTGACTATTGGGGTCAGGGCACACAAGTCACGGTCTCAAGCGGCGGAAGCT
CTGGTGAACAGAAACTGATTTCAGAAG
DYCMDCF#PYSFK#SANILHLSVI#evqlvasggglvqagdslrlscaasgDYCMDCFmgwfrqapgkerefvaaitPYSFKtyyadsvkgrftisrddarntvylqmnslkpedtavyycaa
SANILHLSVInywgqgtqvtvss#AGAAGGAGATATACCATGGAAGTTCAACTGGTTGCATCTGGTGGCGGTTTAGTTCAAGCCGGTGATAGCCTTCGTCTGAGC
TGCGCCGCAAGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTTCACTATTTCACGCGATGATGCGCGTAATACCGTGTACTTACAGATGAACTCACTGAAACCTGAGG
ATACCGCCGTTTATTATTGTGCGGCAAGCGCGAACATTCTCCATTTGTCTGTTATCAACTATTGGGGTCAGGGCACACAAGTCACGGTCTCAAGCGGCGGAA
GCTCTGGTGAACAGAAACTGATTTCAGAAG
DYCMDCF#PYSFK#GRLRLTGSGC#evqlvesggglvqagdslrlscaasgDYCMDCFmgwfrqapgkerefvaaitPYSFKtyyadsvkgrftisrddarntvylqmnslkpedtavyyca
aGRLRLTGSGCdywgqgtqvtvss#ACCATGGAAGTTCAACTGGTTGAATCTGGTGGCGGTTTAGTTCAAGCCGGTGATAGCCTTCGTCTGAGCTGCGCCGCAA
GCGGAGATTACTGCATGGATTGCTTTATGGGTTGGTTTCGCCAGGCACCTGGTAAAGAACGTGAATTTGTGGCAGCCATTACCCCCTATTCCTTTAAGACCTA
CTATGCCGATTCTGTTAAAGGTCGCTTCACTATTTCACGCGATGATGCGCGTAATACCGTGTACTTACAGATGAACTCACTGAAACCTGAGGATACCGCCGTT
TATTATTGTGCGGCAGGTCGCCTTCGCCTCACTGGCAGTGGCTGTGACTATTGGGGTCAGGGCACACAAGTCACGGTCTCAAGCGGCGGAAGCTCTGGTGA
ACAGAAACTGATTTCAGAAG
DYCMDCF#PYSFK#VPNVTWSERA#evqlvesggglvqagdslrlscaasgDYCMDCFmgwfrqapgkerefvaaitPYSFKtyyadsvkgrftisrddarntvylqmnslkpedtavyyc
aaVPNVTWSERAdywgqgtqvtass#ACCATGGAAGTTCAACTGGTTGAATCTGGTGGCGGTTTAGTTCAAGCCGGTGATAGCCTTCGTCTGAGCTGCGCCGCA
AGCGGAGATTACTGCATGGATTGCTTTATGGGTTGGTTTCGCCAGGCACCTGGTAAAGAACGTGAATTTGTGGCAGCCATTACCCCCTATTCCTTTAAGACCT
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTGCGGCAGTCCCCAATGTGACCTGGTCCGAGAGGGCTGACTATTGGGGTCAGGGCACACAAGTCACGGCCTCAAGCGGCGGAAGCTCTGGTG
AACAGAAACTGATTTCAGAAG
DYCMDCF#PYSFK#PLNIGTATIA#evqlvesggvlvqagdslrlscaasgDYCMDCFmgwfrqapgkerefvaaitPYSFKtyyadsvkgrftisrddarntvylqmnslkpedtavyycaa
PLNIGTATIAaywgqgtqvtvss#ACCATGGAAGTTCAACTGGTTGAATCTGGTGGCGTTTTAGTTCAAGCCGGTGATAGCCTTCGTCTGAGCTGCGCCGCAAGC
GGAGATTACTGCATGGATTGCTTTATGGGTTGGTTTCGCCAGGCACCTGGTAAAGAACGTGAATTTGTGGCAGCCATTACCCCCTATTCCTTTAAGACCTACT
ATGCCGATTCTGTTAAAGGTCGCTTCACTATTTCACGCGATGATGCGCGTAATACCGTGTACTTACAGATGAACTCACTGAAACCTGAGGATACCGCCGTTTA
TTATTGTGCGGCACCCCTCAATATCGGCACCGCGACGATTGCCGCCTATTGGGGTCAGGGCACACAAGTCACGGTCTCAAGCGGCGGAAGCTCTGGTGAAC
AGAAACTGATTTCAGAAG
DYCMDCF#PYSFK#DSLILANSQW#evqlvesggglvqagdslrlscaasgDYCMDCFmgwfrqapgkerefvaaitPYSFKtyyadsvkgrftisrddvrntvylqmnslkpedtavyyca
aDSLILANSQWdywgqgtqvtvss#GGAGATATACCATGGAAGTTCAACTGGTTGAATCTGGTGGCGGTTTAGTTCAAGCCGGTGATAGCCTTCGTCTGAGCTG
CGCCGCAAGCGGAGATTACTGCATGGATTGCTTTATGGGTTGGTTTCGCCAGGCACCTGGTAAAGAACGTGAATTTGTGGCAGCCATTACCCCCTATTCCTT
TAAGACCTACTATGCCGATTCTGTTAAAGGTCGCTTCACTATTTCACGCGATGATGTGCGTAATACCGTGTACTTACAGATGAACTCACTGAAACCTGAGGAT
ACCGCCGTTTATTATTGTGCGGCAGACAGCCTTATTTTGGCTAATTCTCAGTGGGACTATTGGGGTCAGGGCACACAAGTCACGGTCTCAAGCGGCGGAAG
CTCTGGTGAACAGAAACTGATTTCAGAAG
DYCMDCF#PYSFK#LFPPATARWE#evqlvesggglvqagdslrlscaasgDYCMDCFmgwfrqapgkerefvaaitPYSFKtyyadsvkgrftisrddarntvylqmnslkpedtavyyc
atLFPPATARWEdywgqgtqvtvss#ATACCATGGAAGTTCAACTGGTTGAATCTGGTGGCGGTTTAGTTCAAGCCGGTGATAGCCTTCGTCTGAGCTGCGCCGC
AAGCGGAGATTACTGCATGGATTGCTTTATGGGTTGGTTTCGCCAGGCACCTGGTAAAGAACGTGAATTTGTGGCAGCCATTACCCCCTATTCCTTTAAGAC
CTACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGACACTTTTTCCCCCGGCGACTGCTAGGTGGGAGGACTATTGGGGTCAGGGCACACAAGTCACGGTCTCAAGCGGCGGAAGCTCTGG
TGAACAGAAACTGATTTCAGAAG
DYCMDCF#PYSFK#LETTKHGCQT#evqlvesggglvqagdslrlscaasgDYCMDCFmgwfrqapgkerefvaaitPYSFKtyyadsvkgrftisrddarntvylqmnslkpedtavyyca
aLETTKHGCQTdswgqgtqvtvss#ATACCATGGAAGTTCAACTGGTTGAATCTGGTGGCGGTCTAGTTCAAGCCGGTGATAGCCTTCGTCTGAGCTGCGCCGCA
AGCGGAGATTACTGCATGGATTGCTTTATGGGTTGGTTTCGCCAGGCACCTGGTAAAGAACGTGAATTTGTGGCAGCCATTACCCCCTATTCCTTTAAGACCT
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTGCGGCACTCGAGACTACTAAGCACGGGTGCCAGACGGACTCTTGGGGTCAGGGCACACAAGTCACGGTCTCAAGCGGCGGAAGCTCTGGT
GAACAGAAACTGATTTCAGAAG
DYCMDCF#PYSFK#NPLDPNWTNS#evqlvesggglvhagdrlrlscaasgDYCMDCFmgwfrqapgkerefvaaitPYSFKtyyadsvkgrftisrddarntvylqmnslkpedtavyyc
aaNPLDPNWTNSdywgqgtqvtvss#GAAGGAGATATACCATGGAAGTTCAACTGGTTGAATCTGGTGGCGGTTTAGTTCACGCCGGTGATCGCCTTCGTCTGA
GCTGCGCCGCAAGCGGAGATTACTGCATGGATTGCTTTATGGGTTGGTTTCGCCAGGCACCTGGTAAAGAACGTGAATTTGTGGCAGCCATTACCCCCTATT
CCTTTAAGACCTACTATGCCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGGCAAACCCTCTGGACCCCAACTGGACTAATTCTGACTATTGGGGTCAGGGCACACAAGTCACGGTCTCAAGCGGCG
GAAGCTCTGGTGAACAGAAACTGATTTCAGAAG
DYCMDCF#PYSFK#DSLILANSQW#eiqlvesggglvqagdslrlscaasgDYCMDCFmgwfrqapgkerefvaaitPYSFKtyyadsvkgrftisrddarntvylqmnslkpedtavyyca
aDSLILANSQWdywgqgtqvtvsg#AGGAGATATACCATGGAAATTCAACTGGTTGAATCTGGTGGCGGTTTAGTTCAAGCCGGTGATAGCCTTCGTCTGAGCT
GCGCCGCAAGCGGAGATTACTGCATGGATTGCTTTATGGGTTGGTTTCGCCAGGCACCTGGTAAAGAACGTGAATTTGTGGCAGCCATTACCCCCTATTCCT
TTAAGACCTACTATGCCGATTCTGTTAAAGGTCGCTTCACTATTTCACGCGATGATGCGCGTAATACCGTGTACTTACAGATGAACTCACTGAAACCTGAGGA
TACCGCCGTTTATTATTGTGCGGCAGACAGCCTTATTTTGGCTAATTCTCAGTGGGACTATTGGGGTCAGGGCACACAAGTCACGGTCTCAGGCGGCGGAA
GCTCTGGTGAACAGAAACTGATTTCAGAAG
DYCMDCF#PYSFK#GDPMIPKLWDSGR#evqlvesggglvqagdslrlscaasgDYCMDCFmgwfrqapgkerefvaaitPYSFKtyyadsvkgrftisrddarntvylqmnslkpedta
vyycarGDPMIPKLWDSGRdywgqgtqvtvss#AGGAGATATACCATGGAAGTTCAACTGGTTGAATCTGGTGGCGGTTTAGTTCAAGCCGGTGATAGCCTTCGT
CTGAGCTGCGCCGCAAGCGGAGATTACTGCATGGATTGCTTTATGGGTTGGTTTCGCCAGGCACCTGGTAAAGAACGTGAATTTGTGGCAGCCATTACCCCC
TATTCCTTTAAGACCTACTATGCCGATTCTGTTAAAGGTCGCTTCACTATTTCACGCGATGATGCGCGTAATACCGTGTACTTACAGATGAACTCACTGAAACC
TGAGGATACCGCCGTTTATTATTGTGCGCGTGGGGACCCGATGATCCCGAAGTTGTGGGATTCGGGGCGTGACTATTGGGGTCAGGGCACACAAGTCACG
GTCTCAAGCGGCGGAAGCTCTGGTGAACAGAAACTGATTTCAGAAG
DYCMDCF#PYSFK#QVSLVTHGDL#evqlvdsggglvhagdrlrlscaasgDYCMDCFmgwfrqapgkerefvaaitPYSFKtyyadsvkgrftisrddarntvylqmnslkpedtavyyc
aaQVSLVTHGDLdswgqgtqvtvss#GAAGGAGATATACCATGGAAGTTCAACTGGTTGACTCTGGTGGCGGTTTAGTTCACGCCGGTGATCGCCTTCGTCTGA
GCTGCGCCGCAAGCGGAGATTACTGCATGGATTGCTTTATGGGTTGGTTTCGCCAGGCACCTGGTAAAGAACGTGAATTTGTGGCAGCCATTACCCCCTATT
CCTTTAAGACCTACTATGCCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGGCACAGGTCTCCCTTGTTACCCATGGGGATCTGGACTCTTGGGGTCAGGGCACACAAGTCACGGTCTCAAGCGGCG
GAAGCTCTGGTGAACAGAAACTGATTTCAGAAG
DYCMDCF#PYSFK#NPLDPNWTNS#evqlvesggglvqvgdslrlscaasgDYCMDCFmgwfrqapgkerefvaaitPYSFKtyyadsvkgrftisrddarntvylqmnslkpedtavyyc
aaNPLDPNWTNSdywgqgtqvtvss#ATACCATGGAAGTTCAACTGGTTGAATCTGGTGGCGGTTTAGTTCAAGTCGGTGATAGCCTTCGTCTGAGCTGCGCCG
CAAGCGGAGATTACTGCATGGATTGCTTTATGGGTTGGTTTCGCCAGGCACCTGGTAAAGAACGTGAATTTGTGGCAGCCATTACCCCCTATTCCTTTAAGA
CCTACTATGCCGATTCTGTTAAAGGTCGCTTCACTATTTCACGCGATGATGCGCGTAATACCGTGTACTTACAGATGAACTCACTGAAACCTGAGGATACCGC
CGTTTATTATTGTGCGGCAAACCCTCTGGACCCCAACTGGACTAATTCTGACTATTGGGGTCAGGGCACACAAGTCACGGTCTCAAGCGGCGGAAGCTCTGG
TGAACAGAAACTGATTTCAGAAG
DYCMDCF#PYSFK#NPLDPNWTNS#evqlvesggglvqagdslrlscaaggDYCMDCFmgwfrqapgkerefvaaitPYSFKtyyadsvkgrftisrddarntvylqmnslkpedtavyy
caaNPLDPNWTNSdywgqgsqvtvss#ATACCATGGAAGTTCAACTGGTTGAATCTGGTGGCGGTTTAGTTCAAGCCGGTGATAGCCTTCGTCTGAGCTGCGCC
GCAGGCGGAGATTACTGCATGGATTGCTTTATGGGTTGGTTTCGCCAGGCACCTGGTAAAGAACGTGAATTTGTGGCAGCCATTACCCCCTATTCCTTTAAG
ACCTACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTGCGGCAAACCCTCTGGACCCCAACTGGACTAATTCTGACTATTGGGGTCAGGGCTCACAAGTCACGGTCTCAAGCGGCGGAAGCTCTG
GTGAACAGAAACTGATTTCAGAAG
DYCMDCF#PYSFK#SMYLNNISIS#evrlvesggglvqagdslrlscaasgDYCMDCFmgwfrqapgkerefvaaitPYSFKtyyadsvkgrftisrddarntvylqmnslkpedtavyycaa
SMYLNNISISdywgqgtqvtvss#ACCATGGAAGTTCGACTGGTTGAATCTGGTGGCGGTTTAGTTCAAGCCGGTGATAGCCTTCGTCTGAGCTGCGCCGCAAGC
GGAGATTACTGCATGGATTGCTTTATGGGTTGGTTTCGCCAGGCACCTGGTAAAGAACGTGAATTTGTGGCAGCCATTACCCCCTATTCCTTTAAGACCTACT
ATGCCGATTCTGTTAAAGGTCGCTTCACTATTTCACGCGATGATGCGCGTAATACCGTGTACTTACAGATGAACTCACTGAAACCTGAGGATACCGCCGTTTA
TTATTGTGCGGCATCTATGTACCTGAACAATATCAGCATTAGTGACTATTGGGGTCAGGGCACACAAGTCACGGTCTCAAGCGGCGGAAGCTCTGGTGAAC
AGAAACTGATTTCAGAAGCCAA
DYCMDCF#PYSFK#PLNIGTATIA#evqlvesggglvqagdslrlscaasgDYCMDCFmgwfrqapgkerefvaaitPYSFKtyyadsvkgrftisrddarntvylqmnslkpedtavyycaa
PLNIGTATIAdywgqgtqgtvss#ATACCATGGAAGTTCAACTGGTTGAATCTGGTGGCGGTTTAGTTCAAGCCGGTGATAGCCTTCGTCTGAGCTGCGCCGCAA
GCGGAGATTACTGCATGGATTGCTTTATGGGTTGGTTTCGCCAGGCACCTGGTAAAGAACGTGAATTTGTGGCAGCCATTACCCCCTATTCCTTTAAGACCTA
CTATGCCGATTCTGTTAAAGGTCGCTTCACTATTTCACGCGATGATGCGCGTAATACCGTGTACTTACAGATGAACTCACTGAAACCTGAGGATACCGCCGTT
TATTATTGTGCGGCACCCCTCAATATCGGCACCGCGACGATTGCCGACTATTGGGGTCAGGGCACACAAGGCACGGTCTCAAGCGGCGGAAGCTCTGGTGA
ACAGAAACTGATTTCAGAAGCCAA
DYCMDCF#PYSFK#NPLDPNWTNS#evqlvesggglvqagdslrlscaasgDYCMDCFmgwfrqapgkerefvaaitPYSFKtyyddsvkgrftisrddarntvylqmnslkpedtavyyc
aaNPLDPNWTNSdywgqgtqgsvss#AGGAGATATACCATGGAAGTTCAACTGGTTGAATCTGGTGGCGGTTTAGTTCAAGCCGGTGATAGCCTTCGTCTGAG
CTGCGCCGCAAGCGGAGATTACTGCATGGATTGCTTTATGGGTTGGTTTCGCCAGGCACCTGGTAAAGAACGTGAATTTGTGGCAGCCATTACCCCCTATTC
CTTTAAGACCTACTATGACGATTCTGTTAAAGGTCGCTTCACTATTTCACGCGATGATGCGCGTAATACCGTGTACTTACAGATGAACTCACTGAAACCTGAG
GATACCGCCGTTTATTATTGTGCGGCAAACCCTCTGGACCCCAACTGGACTAATTCTGACTATTGGGGTCAGGGCACACAAGGCTCGGTCTCAAGCGGCGG
AAGCTCTGGTGAACAGAAACTGATTTCAGAAGCCAA
DYCMDCF#PYSFK#QVSLVTHGDL#evqlvesggglvqagdslrlscaasgDYCMDCFmgwfrqapgkerefvaaitPYSFKtyyadsvkgrftisrddarntvylqmnslkpedtavyyc
aaQVSLVTHGDLdywgqgtqgtvss#AGGAGATATACCATGGAAGTTCAACTGGTTGAATCTGGTGGCGGTTTAGTTCAAGCCGGTGATAGCCTTCGTCTGAGC
TGCGCCGCAAGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTTCACTATTTCACGCGATGATGCGCGTAATACCGTGTACTTACAGATGAACTCACTGAAACCTGAGG
ATACCGCCGTTTATTATTGTGCGGCACAGGTCTCCCTTGTTACCCATGGGGATCTGGACTATTGGGGTCAGGGCACACAAGGCACGGTCTCAAGCGGCGGA
AGCTCTGGTGAACAGAAACTGATTTCAGAAGCCAA
DYCMDCF#PYSFK#NPLDPNWTNS#evqlvesggglvqagdslrlscaaggDYCMDCFmgwfrqapgkerefvaaitPYSFKtyyadsvkgrftisrddarntvylqmnslkpedtavyy
caaNPLDPNWTNSdywgqgtqgtvss#GGAGATATACCATGGAAGTTCAACTGGTTGAATCTGGTGGCGGTTTAGTTCAAGCCGGTGATAGCCTTCGTCTGAGC
TGCGCCGCAGGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTTCACTATTTCACGCGATGATGCGCGTAATACCGTGTACTTACAGATGAACTCACTGAAACCTGAGG
ATACCGCCGTTTATTATTGTGCGGCAAACCCTCTGGACCCCAACTGGACTAATTCTGACTATTGGGGTCAGGGCACACAAGGCACGGTCTCAAGCGGCGGA
AGCTCTGGTGAACAGAAACTGATTTCAGAAGCCAA
DYCMDCF#PYSFK#PLNIGTATIA#evqlvesggglvqagdslrlscaasgDYCMDCFmgwfrqapgkerefvaaitPYSFKtyyvdsvkgrftisrddarntvylqmnslkpedtavyycaa
PLNIGTATIAdywgqgtqvtvss#AGATATACCATGGAAGTTCAACTGGTTGAATCTGGTGGCGGTTTAGTTCAAGCCGGTGATAGCCTTCGTCTGAGCTGCGCC
GCAAGCGGAGATTACTGCATGGATTGCTTTATGGGTTGGTTTCGCCAGGCACCTGGTAAAGAACGTGAATTTGTGGCAGCCATTACCCCCTATTCCTTTAAG
ACCTACTATGTCGATTCTGTTAAAGGTCGCTTCACTATTTCACGCGATGATGCGCGTAATACCGTGTACTTACAGATGAACTCACTGAAACCTGAGGATACCG
CCGTTTATTATTGTGCGGCACCCCTCAATATCGGCACCGCGACGATTGCCGACTATTGGGGTCAGGGCACACAAGTCACGGTCTCAAGCGGCGGAAGCTCT
GGTGAACAGAAACTGATTTCAGAAGCCAA
DYCMDCF#PYSFK#LFPPATARWE#evqlvesggglvqagdslrlscatsgDYCMDCFmgwfrqapgkerefvaaitPYSFKtyyadsvkgrftisrddarntvylqmnslkpedtavyyca
tLFPPATARWEdywgqgtqvtvss#AGGAGATATACCATGGAAGTTCAACTGGTTGAATCTGGTGGCGGTTTAGTTCAAGCCGGTGATAGCCTTCGTCTGAGCT
GCGCCACAAGCGGAGATTACTGCATGGATTGCTTTATGGGTTGGTTTCGCCAGGCACCTGGTAAAGAACGTGAATTTGTGGCAGCCATTACCCCCTATTCCT
TTAAGACCTACTATGCCGATTCTGTTAAAGGTCGCTTCACTATTTCACGCGATGATGCGCGTAATACCGTGTACTTACAGATGAACTCACTGAAACCTGAGGA
TACCGCCGTTTATTATTGTGCGACACTTTTTCCCCCGGCGACTGCTAGGTGGGAGGACTATTGGGGTCAGGGCACACAAGTCACGGTCTCAAGCGGCGGAA
GCTCTGGTGAACAGAAACTGATTTCAGAAGCCAA
DYCMDCF#PYSFK#NPLDPNWTNS#evqlvesggglvhagdslrlscaasgDYCMDCFmgwfrqapgkerefvaaitPYSFKtyyadsvkgrftisrddarntvylqmnslkpedtavyyc
aaNPLDPNWTNSdywgqgtqgtvss#AGATATACCATGGAAGTTCAACTGGTTGAATCTGGTGGCGGTTTAGTTCACGCCGGTGATAGCCTTCGTCTGAGCTGC
GCCGCAAGCGGAGATTACTGCATGGATTGCTTTATGGGTTGGTTTCGCCAGGCACCTGGTAAAGAACGTGAATTTGTGGCAGCCATTACCCCCTATTCCTTT
AAGACCTACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGGCAAACCCTCTGGACCCCAACTGGACTAATTCTGACTATTGGGGGCAGGGCACACAAGGCACGGTCTCAAGCGGCGGAAGC
TCTGGTGAACAGAAACTGATTTCAGAAGCCAA
DYCMDCF#PYSFK#QVSLVTHGDL#evqlvesggglvqagdslrlscaasgDYCMDCFmgwfrqapgkerefaaaitPYSFKtyyadsvkgrftisrddarntvylqmnslkpedtavyyc
aaQVSLVTHGDLdswgqgtqvtvss#ACCATGGAAGTTCAACTGGTTGAATCTGGTGGCGGTTTAGTTCAAGCCGGTGATAGCCTTCGTCTGAGCTGCGCCGCA
AGCGGAGATTACTGCATGGATTGCTTTATGGGTTGGTTTCGCCAGGCACCTGGTAAAGAACGTGAATTTGCGGCAGCCATTACCCCCTATTCCTTTAAGACC
TACTATGCCGATTCTGTTAAAGGTCGCTTCACTATTTCACGCGATGATGCGCGTAATACCGTGTACTTACAGATGAACTCACTGAAACCTGAGGATACCGCCG
TTTATTATTGTGCGGCTCAGGTCTCCCTTGTTACCCATGGGGATCTGGACTCTTGGGGTCAGGGCACACAAGTCACGGTCTCAAGCGGCGGAAGCTCTGGTG
AACAGAAACTGATTTCAGAAGCCAA
DYCMDCF#PYSFK#NPLDPNWTNS#evqlvdsggglvqagdrlrlscaasgDYCMDCFmgwfrqapgkerefvaaitPYSFKtyyadsvkgrftisrddarntvylqmnslkpedtavyyc
aaNPLDPNWTNSdywgqgtqvtvss#GAAGGAGATATACCATGGAAGTTCAACTGGTTGACTCTGGTGGCGGTTTAGTTCAAGCCGGTGATCGCCTTCGTCTGA
GCTGCGCCGCAAGCGGAGATTACTGCATGGATTGCTTTATGGGTTGGTTTCGCCAGGCACCTGGTAAAGAACGTGAATTTGTGGCAGCCATTACCCCCTATT
CCTTTAAGACCTACTATGCCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGGCAAACCCTCTGGACCCCAACTGGACTAATTCTGACTATTGGGGTCAGGGCACACAAGTCACGGTCTCAAGCGGCG
GAAGCTCTGGTGAACAGAAACTGATTTCAGAAGCCAA
DSCMDCF#PYSFK#DSLILANSQW#evqlvesggglvqagdslrlscaasgDSCMDCFmgwfrqapgkerefvaaitPYSFKtyyadsvkgrftisrddarntvylqmnslkpedtavyyca
aDSLILANSQWdywgqgtqvtvss#ATACCATGGAAGTTCAACTGGTTGAATCTGGTGGCGGTTTAGTTCAAGCCGGTGATAGCCTTCGTCTGAGCTGCGCCGC
AAGCGGAGATTCCTGCATGGATTGCTTTATGGGTTGGTTTCGCCAGGCACCTGGTAAAGAACGTGAATTTGTGGCAGCCATTACCCCCTATTCCTTTAAGAC
CTACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGCAGACAGCCTTATTTTGGCTAATTCTCAGTGGGACTATTGGGGTCAGGGCACACAAGTCACGGTCTCAAGCGGCGGAAGCTCTGGT
GAACAGAAACTGATTTCAGAAGCCAA
DYCMDCF#PYSFK#LETTKHGCQT#evqlvesggglvqagdslrlscaasgDYCMDCFmgwfrqapgkerefvaaitPYSFKtyyadsvkgrftisrddarntvylqmnslkpedtavyyca
aLETTKHGCQTdywgqgtqvtvss#AAGGAGATATACCATGGAAGTTCAACTGGTTGAATCTGGTGGCGGTTTAGTTCAAGCCGGTGATAGCCTTCGTCTGAGC
TGCGCCGCAAGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTTCACTATTTCACGCGATGATGCGCGTAATACCGTGTACTTACAGATGAACTCACTGAAACCTGAGG
ATACCGCCGTTTATTATTGTGCGGCACTCGAGACTACTAAGCACGGGTGCCAGACGGACTATTGGGGTCAGGGCACACAAGTCACGGTCTCAAGCGGCGGA
AGCTCTGGTGAACAGAAACTGATTTCAGAAGCCAA
DYCMDCF#PYSFK#TASDTSDYGM#evqlvesggglvqagdslrlscaasgDYCMDCFmgwfrqapgkerefvaaitPYSFKtyyadsvkgrftisrddarntvylqmnslkpedtavyyc
atTASDTSDYGMdywgqgtqvtvss#GAAGGAGATATACCATGGAAGTTCAACTGGTTGAATCTGGTGGCGGTTTAGTTCAAGCCGGTGATAGCCTTCGTCTGA
GCTGCGCCGCAAGCGGAGATTACTGCATGGATTGCTTTATGGGTTGGTTTCGCCAGGCACCTGGTAAAGAACGTGAATTTGTGGCAGCCATTACCCCCTATT
CCTTTAAGACCTACTATGCCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGACAACCGCCTCCGATACTAGTGATTACGGTATGGACTATTGGGGTCAGGGCACACAAGTCACGGTCTCAAGCGGCG
GAAGCTCTGGTGAACAGAAACTGATTTCAGAAGCCAA
DYCMDCF#PYSFK#PLNIGTATIA#evqlvesggglvqagdslrlscaasgDYCMDCFmgwfrrapgkerefvaaitPYSFKtyyadsvkgrftisrddarntvylqmnslkpedtavyycaa
PLNIGTATIAdywgqgtqvtvss#ATATACCATGGAAGTTCAACTGGTTGAATCTGGTGGCGGTTTAGTTCAAGCCGGTGATAGCCTTCGTCTGAGCTGCGCCGC
AAGCGGAGATTACTGCATGGATTGCTTTATGGGTTGGTTTCGCCGGGCACCTGGTAAAGAACGTGAATTTGTGGCAGCCATTACCCCCTATTCCTTTAAGAC
CTACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGCACCCCTCAATATCGGCACCGCGACGATTGCCGACTATTGGGGTCAGGGCACACAAGTCACGGTCTCAAGCGGCGGAAGCTCTGG
TGAACAGAAACTGATTTCAGAAGCCAA
DYCMDCF#PYSFK#PLNIGTATIA#evqlvesggglvqagdslrlscaasgDYCMDCFmgwfrqapgkerefvaaitPYSFKtyyadsvkgrftisrddarntvylqmnslkpedtavyycaa
PLNIGTATIAdywgqgtqvmvss#GAAGGAGATATACCATGGAAGTTCAACTGGTTGAATCTGGTGGCGGTTTAGTTCAAGCCGGTGATAGCCTTCGTCTGAGC
TGCGCCGCAAGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTTCACTATTTCACGCGATGATGCGCGTAATACCGTGTACTTACAGATGAACTCACTGAAACCTGAGG
ATACCGCCGTTTATTATTGTGCGGCACCCCTCAATATCGGCACCGCGACGATTGCCGACTATTGGGGTCAGGGCACACAAGTCATGGTCTCAAGCGGCGGA
AGCTCTGGTGAACAGAAACTGATTTCAGAAGCCAA
DYCMDCF#PYSFK#DSLILANSQW#evqlvesggglvqagdslrlscaasgDYCMDCFmgwfrqapgkerefvaaitPYSFKtyyadsvkgrftisrddarntvylqmnslkpedtavyyca
aDSLILANSQWdywgqgtqvtfss#ATACCATGGAAGTTCAACTGGTTGAATCTGGTGGCGGTTTAGTTCAAGCCGGTGATAGCCTTCGTCTGAGCTGCGCCGCA
AGCGGAGATTACTGCATGGATTGCTTTATGGGTTGGTTTCGCCAGGCACCTGGTAAAGAACGTGAATTTGTGGCAGCCATTACCCCCTATTCCTTTAAGACCT
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTGCGGCAGACAGCCTTATTTTGGCTAATTCTCAGTGGGACTATTGGGGTCAGGGCACACAAGTCACGTTCTCAAGCGGCGGAAGCTCTGGTGA
ACAGAAACTGATTTCAGAAGCCAA
DYCMDCF#PYSFK#LETTKHGCQT#evqlvesggglvqagdslrlscaasgDYCMDCFmgwfrqapgkerefvaaitPYSFKtyyadsvkgrftisrddarntvylqmnslkpedtavyyca
aLETTKHGCQTdswgqgtqvtfss#ATATACCATGGAAGTTCAACTGGTTGAATCTGGTGGCGGTTTAGTTCAAGCCGGTGATAGCCTTCGTCTGAGCTGCGCCG
CAAGCGGAGATTACTGCATGGATTGCTTTATGGGTTGGTTTCGCCAGGCACCTGGTAAAGAACGTGAATTTGTGGCAGCCATTACCCCCTATTCCTTTAAGA
CCTACTATGCCGATTCTGTTAAAGGTCGCTTCACTATTTCACGCGATGATGCGCGTAATACCGTGTACTTACAGATGAACTCACTGAAACCTGAGGATACCGC
CGTTTATTATTGTGCGGCACTCGAGACTACTAAGCACGGGTGCCAGACGGACTCTTGGGGTCAGGGCACACAAGTCACGTTCTCAAGCGGCGGAAGCTCTG
GTGAACAGAAACTGATTTCAGAAGCCAA
DYCMDCF#PYSFK#DSLILANSQW#evqlvesggglvqagdslrlscaasgDYCMDCFmgwfrqapgkerefvaaitPYSFKtyyadsvkgrftisrddarntvylqmnslkpedtavyyca
tDSLILANSQWdywgqgtqvtvss#AAGGAGATATACCATGGAAGTTCAACTGGTTGAATCTGGTGGCGGTTTAGTTCAAGCCGGTGATAGCCTTCGTCTGAGC
TGCGCCGCAAGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTTCACTATTTCACGCGATGATGCGCGTAATACCGTGTACTTACAGATGAACTCACTGAAACCTGAGG
ATACCGCCGTTTATTATTGTGCGACAGACAGCCTTATTTTGGCTAATTCTCAGTGGGACTATTGGGGTCAGGGCACACAAGTCACGGTCTCAAGCGGCGGAA
GCTCTGGTGAACAGAAACTGATTTCAGAAGCCAA
DYCMDCF#PYSFK#DSLILANSQW#evqlvdsggglvqagdrlrlscaasgDYCMDCFmgwfrqapgkerefvaaitPYSFKtyyadsvkgrftisrddarntvylqmnslkpedtavyyca
aDSLILANSQWdywgqgtqvtvss#GAAGGAGATATACCATGGAAGTTCAACTGGTTGACTCTGGTGGCGGTTTAGTTCAAGCCGGTGATCGCCTTCGTCTGAG
CTGCGCCGCAAGCGGAGATTACTGCATGGATTGCTTTATGGGTTGGTTTCGCCAGGCACCTGGTAAAGAACGTGAATTTGTGGCAGCCATTACCCCCTATTC
CTTTAAGACCTACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTGCGGCAGACAGCCTTATTTTGGCTAATTCTCAGTGGGACTATTGGGGTCAGGGCACACAAGTCACGGTCTCAAGCGGCGGA
AGCTCTGGTGAACAGAAACTGATTTCAGAAGCCAA
DYCMDCF#PYSFK#PLNIGTATIA#evqlvesggglvqagdslrlscaasgDYCMDCFmgwfrqapgkerefvaaitPYSFKtyyadsvkgrftisrddarntvylqmnslkpedtavyycaa
PLNIGTATIAdywgqgtqvtfss#AAGGAGATATACCATGGAAGTTCAACTGGTTGAATCTGGTGGCGGTTTAGTTCAAGCCGGTGATAGCCTTCGTCTGAGCTG
TGCCGCAAGCGGAGATTACTGCATGGATTGCTTTATGGGTTGGTTTCGCCAGGCACCTGGTAAAGAACGTGAATTTGTGGCAGCCATTACCCCCTATTCCTTT
AAGACCTACTATGCCGATTCCGTTAAAGGTCGCTTCACTATTTCACGCGATGATGCGCGTAATACCGTGTACTTACAGATGAACTCACTGAAACCTGAGGAT
ACCGCCGTTTATTATTGTGCGGCACCCCTCAATATCGGCACCGCGACGATTGCCGACTATTGGGGTCAGGGCACACAAGTCACGTTCTCAAGCGGCGGAAG
CTCTGGTGAACAGAAACTGATTTCAGAAGCCAA
DYCMDCF#PYSFK#WPSLPTGGTP#evqlvesggglvqagdslrlscaasgDYCMDCFmgwfrqapgkehefvaaitPYSFKtyyadsvkgrftisrddarntvylqmnslkpedtavyyc
atWPSLPTGGTPdywgqgtqvtvss#ATATACCATGGAAGTTCAACTGGTTGAATCTGGTGGCGGTTTAGTTCAAGCCGGTGATAGCCTTCGTCTGAGCTGCGCC
GCAAGCGGAGATTACTGCATGGATTGCTTTATGGGTTGGTTTCGCCAGGCACCTGGTAAAGAACATGAATTTGTGGCAGCCATTACCCCCTATTCCTTTAAG
ACCTACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTGCGACATGGCCGTCCCTGCCTACGGGCGGTACCCCCGACTATTGGGGTCAGGGCACACAAGTCACGGTCTCAAGCGGCGGAAGCTCT
GGTGAACAGAAACTGATTTCAGAAGCCAA
DYCMDCF#PYSFK#LLPLDGFETS#evqlvesggglvqagdslrlscaasgDYCMDCFmgwfrqapgkerefvaaitPYSFKtyyadcvkgrftisrddarntvylqmnslkpedtavyycaa
LLPLDGFETSdywgqgtqvtvss#ACCATGGAAGTTCAACTGGTTGAATCTGGTGGCGGTTTAGTTCAAGCCGGTGATAGCCTTCGTCTGAGCTGCGCCGCAAGC
GGAGATTACTGCATGGATTGCTTTATGGGTTGGTTTCGCCAGGCACCTGGTAAAGAACGTGAATTTGTGGCAGCCATTACCCCCTATTCCTTTAAGACCTACT
ATGCCGATTGTGTTAAAGGTCGCTTCACTATTTCACGCGATGATGCGCGTAATACCGTGTACTTACAGATGAACTCACTGAAACCTGAGGATACCGCCGTTTA
TTATTGTGCGGCACTTCTCCCCCTGGATGGTTTTGAGACTTCGGACTATTGGGGTCAGGGCACACAAGTCACGGTCTCAAGCGGCGGAAGCTCTGGTGAAC
AGAAACTGATTTCAGAAGCCAA
DYCMDCF#PYSFK#SIQTLIGSSSCWV#evqlvesggglvqagdslrlscaasgDYCMDCFmgwfrqapgkerefvaaitPYSFKtyyadsvkgrftisrddarntvylqmnslkpedtavy
ycnaSIQTLIGSSSCWVdywgqgtqvtvss#AGATATACCATGGAAGTTCAACTGGTTGAATCTGGTGGCGGTTTAGTTCAAGCCGGTGATAGCCTTCGTCTGAGC
TGCGCCGCAAGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTTCACTATTTCACGCGATGATGCGCGTAATACCGTGTACTTACAGATGAACTCACTGAAACCTGAGG
ATACCGCCGTTTATTATTGTAATGCAAGCATCCAGACCCTGATTGGCAGTTCGTCTTGCTGGGTGGACTATTGGGGTCAGGGCACACAAGTCACGGTCTCAA
GCGGCGGAAGCTCTGGTGAACAGAAACTGATTTCAGAAGCCAA
DYCMDCF#PYSFK#PLNIGTATIA#evqlvesggglvqdgdslrlscaasgDYCMDCFmgwfrqapgkerefvaaitPYSFKtyyadsvkgrftisrddarntvylqmnslkpedtavyycaa
PLNIGTATIAdywgqgtqvtvss#ATATACCATGGAAGTTCAACTGGTTGAATCTGGTGGCGGTTTAGTTCAAGACGGTGATAGCCTTCGTCTGAGCTGCGCCGC
AAGCGGAGATTACTGCATGGATTGCTTTATGGGTTGGTTTCGCCAGGCACCTGGTAAAGAACGTGAATTTGTGGCAGCCATTACCCCCTATTCCTTTAAGAC
CTACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGCACCCCTCAATATCGGCACCGCGACGATTGCCGACTATTGGGGTCAGGGCACACAAGTCACGGTCTCAAGCGGCGGAAGCTCTGG
TGAACAGAAACTGATTTCAGAAGCCAA
DYCMDCF#PYSFK#NPLDPNWTNS#evqlvesggglvqagdslrlscaasgDYCMDCFmgwfrqapgkerefveaitPYSFKtyytdsvkgrftisrddarntvylqmnslkpedtavyyc
aaNPLDPNWTNSdywgqgtqvtvss#AAGGAGATATACCATGGAAGTTCAACTGGTTGAATCTGGTGGCGGTTTAGTTCAAGCCGGTGATAGCCTTCGTCTGA
GCTGCGCCGCAAGCGGAGATTACTGCATGGATTGCTTTATGGGTTGGTTTCGCCAGGCACCTGGTAAAGAACGTGAATTTGTGGAAGCCATTACCCCCTATT
CCTTTAAGACCTACTATACCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGGCAAACCCTCTGGACCCCAACTGGACTAATTCTGACTATTGGGGTCAGGGCACACAAGTCACGGTCTCAAGCGGCG
GAAGCTCTGGTGAAAAGAAACTGATTTCAGAAGCCAA
DYCMDCF#ASYRY#LTSPLSDSS#evqlvesggglvqagdslrlscaasgDYCMDCFmgwfrqapgkerefvaaitASYRYtdyadsvrgrfsisadsakntvylqmnslkpedtavyycaa
LTSPLSDSSdywgqgtqvtvss#ATACCATGGAAGTTCAACTGGTTGAATCTGGTGGCGGTTTAGTTCAAGCCGGTGATAGCCTTCGTCTGAGCTGCGCCGCAAG
CGGAGATTACTGCATGGATTGCTTTATGGGTTGGTTTCGCCAGGCACCTGGTAAAGAACGTGAATTTGTGGCAGCCATTACCGCGTCTTACCGTTACACCGA
CTATGCCGATTCTGTTCGTGGTCGCTTCAGCATTTCAGCAGATTCTGCGAAGAATACCGTGTACTTACAGATGAACTCACTGAAACCTGAGGATACCGCCGTT
TATTATTGTGCGGCATTGACGTCTCCGCTTTCGGATTCGTCCGACTATTGGGGTCAGGGCACACAAGTCACGGTCTCAAGCGGCGGAAGCTCTGGTGAACA
GAAACTGATTTCAGAAGCCAA
DYCMDCF#PYSFK#QVSLVTHGDL#evqlvesggglvqagdslrlscaasgDYCMDCFmgwfrqapgkerefvaaitPYSFKtyyadsvkgrftisrddarntvylqmnslkpedtavyyc
aaQVSLVTHGDLdywgqgtqvtvss#ACCATGGAAGTTCAACTGGTTGAATCTGGTGGCGGTTTAGTTCAAGCCGGTGATAGCCTTCGTCTGAGCTGCGCCGCA
AGCGGAGATTACTGCATGGATTGCTTTATGGGTTGGTTTCGCCAGGCACCTGGTAAAGAACGTGAATTTGTGGCAGCCATTACCCCCTATTCCTTTAAGACCT
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTGCGGCACAGGTCTCCCTTGTTACCCATGGGGATCTGGACTATTGGGGTCAGGGCACACAAGTCACGGTCTCAAGCGGCGGAAGCTCTGGTG
AACAGAAACTGATTTCAGAAGCCAA
DYCMDCF#PYSFK#NPLDPNWTNS#evqlvesggglvqagdslrlscaasgDYCMDCFmgwfrqapgkerefvsaitPYSFKtyyadsvkgrftisrddarntvylqmnslkpedtavyyc
aaNPLDPNWTNSdywgqgtqvtvss#ACCATGGAAGTTCAACTGGTTGAATCTGGTGGCGGTTTAGTTCAAGCCGGTGATAGCCTTCGTCTGAGCTGCGCCGCA
AGCGGAGATTACTGCATGGATTGCTTTATGGGTTGGTTTCGCCAGGCACCTGGTAAAGAACGTGAATTTGTGTCAGCCATTACCCCCTATTCCTTTAAGACCT
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTGCGGCAAACCCTCTGGACCCCAACTGGACTAATTCTGACTATTGGGGTCAGGGCACACAAGTCACGGTCTCAAGCGGCGGAAGCTCTGGTG
AACAGAAACTGATTTCAGAAGCCAA
DYCMDCF#PYSFK#PVISFAADGW#evqlvesggglvqagdslrlscaasgDYCMDCFmgwfrqapgkerefvaaitPYSFKtyyadsvkgrftisrddarntvylqmnslkpedtavyyc
aaPVISFAADGWdywgqgtqvtvss#ATATACCATGGAAGTTCAACTGGTTGAATCTGGTGGCGGTTTAGTTCAAGCCGGTGATAGCCTTCGTCTGAGCTGCGCC
GCAAGCGGAGATTACTGCATGGATTGCTTTATGGGTTGGTTTCGCCAGGCACCTGGTAAAGAACGTGAATTTGTGGCAGCCATTACCCCCTATTCCTTTAAG
ACCTACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTGCGGCACCCGTCATCTCCTTTGCTGCGGACGGCTGGGACTATTGGGGTCAGGGCACACAAGTCACGGTCTCAAGCGGCGGAAGCTCT
GGTGAACAGAAACTGATTTCAGAAGCCAA
DYCMDCF#PYSFK#PLNIGTATIA#evqlvesggglvqagdslrlscaasgDYCMDCFlgwfrqapgkerefvaaitPYSFKtyyadsvkgrftiscddarntvylqmnslkpedtavyycaaP
LNIGTATIAdywgqgtqvtvss#GAAGGAGATATACCATGGAAGTTCAACTGGTTGAATCTGGTGGCGGTTTAGTTCAAGCCGGTGATAGCCTTCGTCTGAGCTG
CGCCGCAAGCGGAGATTACTGCATGGATTGCTTTTTGGGTTGGTTTCGCCAGGCACCTGGTAAAGAACGTGAATTTGTGGCAGCCATTACCCCCTATTCCTTT
AAGACCTACTATGCCGATTCTGTTAAAGGTCGCTTCACTATTTCATGCGATGATGCGCGTAATACCGTGTACTTACAGATGAACTCACTGAAACCTGAGGATA
CCGCCGTTTATTATTGTGCGGCACCCCTCAATATCGGCACCGCGACGATTGCCGACTATTGGGGTCAGGGCACACAAGTCACGGTCTCAAGCGGCGGAAGC
TCTGGTGAACAGAAACTGATTTCAGAAGCCAA
DYCMDCF#PYSFK#DSLILANSQW#evqlvesggglvqagdsprlscaasgDYCMDCFmgwfrqapgkerefvaaitPYSFKtyyadsvkgrftisrddarntvylqmnslkpedtavyyc
aaDSLILANSQWdywgqgtqvtvss#GAAGGAGATATACCATGGAAGTTCAACTGGTTGAATCTGGTGGCGGTTTAGTTCAAGCCGGTGATAGCCCTCGTCTGA
GCTGCGCCGCAAGCGGAGATTACTGCATGGATTGCTTTATGGGTTGGTTTCGCCAGGCACCTGGTAAAGAACGTGAATTTGTGGCAGCCATTACCCCCTATT
CCTTTAAGACCTACTATGCCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGGCAGACAGCCTTATTTTGGCTAATTCTCAGTGGGACTATTGGGGTCAGGGCACACAAGTCACGGTCTCAAGCGGCG
GAAGCTCTGGTGAACAGAAACTGATTTCAGAAGCCAA
DYCMDCF#PYSFK#PLNIGTATIA#evqlvesggglvqagdslrlscaasgDYCMDCFmgwfrqapgkerefvaaitPYSFKtyyadsvkgrftisrddarntvylqmnslkpedtavyycaa
PLNIGTATIAdywghgtqvtvss#AGATATACCATGGAAGTTCAACTGGTTGAATCTGGTGGCGGTTTAGTTCAAGCCGGTGATAGCCTTCGTCTGAGCTGCGCC
GCAAGCGGAGATTACTGCATGGATTGCTTTATGGGTTGGTTTCGCCAGGCACCTGGTAAAGAACGTGAATTTGTGGCAGCCATTACCCCCTATTCCTTTAAG
ACCTACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTGCGGCACCCCTCAATATCGGCACCGCGACGATTGCCGACTACTGGGGTCATGGCACACAAGTCACGGTCTCAAGCGGCGGAAGCTCTG
CTGATCAGAAACTGATTTCAGAAGCCAA
DYCMDCF#PYSFK#DSLILANSQW#evqlvesggglvqagdslrlscaasgDYCMDCFmgwfrqapgkerefvaaitPYSFKtyyadsvkgrftisrddarntvylqmnslkpedtavyyca
tDSLILANSQWdywghgtqvtvss#AAGGAGATATACCATGGAAGTTCAACTGGTTGAATCTGGTGGCGGTTTAGTTCAAGCCGGTGATAGCCTTCGTCTGAGC
TGCGCCGCAAGCGGAGATTACTGCATGGATTGCTTTATGGGTTGGTTTCGCCAGGCACCTGGTAAAGAACGTGAATTTGTGGCAGCCATTACCCCCTATTCC
TTTAAGACCTACTATGCCGATTCTGTTAAAGGTCGCTTCACTATTTCACGCGATGATGCGCGTAATACCGTGTACTTACAGATGAACTCACTGAAACCTGAGG
ATACCGCCGTTTATTATTGTGCGACAGACAGCCTTATTTTGGCTAATTCTCAGTGGGACTATTGGGGTCATGGCACACAAGTCACGGTCTCAAGCGGCGGAA
GCTCTGGTGAACAGAAACTGATTTCAGAAGCCAA
DYCMDCF#PYSFK#DSLILANSQW#evqlvesggglvqagdslrlscaasgDYCMDCFmgwfrqapgkerefvaaitPYSFKtyyadsvkgrftisrddarntvylqmnslkpedtavyyca
aDSLILANSQWdywgqgtqgtvss#ATATACCATGGAAGTTCAACTGGTTGAATCTGGTGGCGGTTTAGTTCAAGCCGGTGATAGCCTTCGTCTGAGCTGCGCC
GCAAGCGGAGATTACTGCATGGATTGCTTTATGGGTTGGTTTCGCCAGGCACCTGGTAAAGAACGTGAATTTGTGGCAGCCATTACCCCCTATTCCTTTAAG
ACCTACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTGCGGCAGACAGCCTTATTTTGGCTAATTCTCAGTGGGACTATTGGGGTCAGGGCACACAAGGCACGGTCTCAAGCGGCGGAAGCTCT
GGTGAACAGAAACTGATTTCAGAAGCCAA
DYCMDCF#PYSFK#QVSLVTHGDL#evqlvesggglvqagdslrlscaasgDYCMDCFmgwfcqapgkerefvaaitPYSFKtyyadsvkgrftisrddarntvylqmnslkpedtavyyc
aaQVSLVTHGDLdswgqgtqvtvss#AAGGAGATATACCATGGAAGTTCAACTGGTTGAATCTGGTGGCGGTTTAGTTCAAGCCGGTGATAGCCTTCGTCTGAG
CTGCGCCGCAAGCGGAGATTACTGCATGGATTGCTTTATGGGTTGGTTTTGCCAGGCACCTGGTAAAGAACGTGAATTTGTGGCAGCCATTACCCCCTATTC
CTTTAAGACCTACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTGCGGCACAGGTCTCCCTTGTTACCCATGGGGATCTGGACTCTTGGGGTCAGGGCACACAAGTCACGGTCTCAAGCGGCGG
AAGCTCTGGTGAACAGAAACTGATTTCAGAAGCCAA
DSCMDCF#PYSFK#NPLDPNWTNS#evqlvesggglvqagdslrlscaasgDSCMDCFmgwfrqapgkerefvaaitPYSFKtyyadsvkgrftisrddarntvylqmnslkpedtavyyc
aaNPLDPNWTNSdywgqgtqvtvss#ATACCATGGAAGTTCAACTGGTTGAATCTGGTGGCGGTTTAGTTCAAGCCGGTGATAGCCTTCGTCTGAGCTGCGCCG
CAAGCGGAGATTCCTGCATGGATTGCTTTATGGGTTGGTTTCGCCAGGCACCTGGTAAAGAACGTGAATTTGTGGCAGCCATTACCCCCTATTCCTTTAAGA
CCTACTATGCCGATTCTGTTAAAGGTCGCTTCACTATTTCACGCGATGATGCGCGTAATACCGTGTACTTACAGATGAACTCACTGAAACCTGAGGATACCGC
CGTTTATTATTGTGCGGCAAACCCTCTGGACCCCAACTGGACTAATTCTGACTATTGGGGTCAGGGCACACAAGTCACGGTCTCAAGCGGCGGAAGCTCTGG
TGAACAGAAACTGATTTCAGAAGCCAA
DYCMDCF#PYSFK#PLNIGTATIA#evqlvesggglvqagdslrlscaaseDYCMDCFmgwfrqapgkerefvaaitPYSFKtyyadsvkgrftisrddarntvylqmnslkpedtavyycaa
PLNIGTATIAdywgqgtqgtvss#ACCATGGAAGTTCAACTGGTTGAATCTGGTGGCGGTTTAGTTCAAGCCGGTGATAGCCTTCGTCTGAGCTGCGCCGCAAGC
GAAGATTACTGCATGGATTGCTTTATGGGTTGGTTTCGCCAGGCACCTGGTAAAGAACGTGAATTTGTGGCAGCCATTACCCCCTATTCCTTTAAGACCTACT
ATGCCGATTCTGTTAAAGGTCGCTTCACTATTTCACGCGATGATGCGCGTAATACCGTGTACTTACAGATGAACTCACTGAAACCTGAGGATACCGCCGTTTA
TTATTGTGCGGCACCCCTCAATATCGGCACCGCGACGATTGCCGACTATTGGGGTCAGGGCACACAAGGCACGGTCTCAAGCGGCGGAAGCTCTGGTGAAC
AGAAACTGATTTCAGAAGCCAA
DYCMDCF#PYSFK#NPLDPNWTNS#evqlvesggglvqagdslrlscaaggDYCMDCFmgwfrqapgkerefvaaitPYSFKtyyadsvkgrftisrddarntvylqmnslkpedtavyy
caaNPLDPNWTNSdywgqgtqvmvss#AAGGAGATATACCATGGAAGTTCAACTGGTTGAATCTGGTGGCGGTTTAGTTCAAGCCGGTGATAGCCTTCGTCTG
AGCTGCGCCGCAGGCGGAGATTACTGCATGGATTGCTTTATGGGTTGGTTTCGCCAGGCACCTGGTAAAGAACGTGAATTTGTGGCAGCCATTACCCCCTAT
TCCTTTAAGACCTACTATGCCGATTCTGTTAAAGGTCGCTTCACTATTTCACGCGATGATGCGCGTAATACCGTGTACTTACAGATGAACTCACTGAAACCTG
AGGATACCGCCGTTTATTATTGTGCGGCAAACCCTCTGGACCCCAACTGGACTAATTCTGACTATTGGGGTCAGGGCACACAAGTCATGGTCTCAAGCGGCG
GAAGCTCTGGTGAACAGAAACTGATTTCAGAAGCCAA
DYCMDCF#PYSFK#NPLDPNWTNS#evqlvesggglvqagdslrlscaasgDYCMDCFmgwfrqapgkerefvaaitPYSFKtyyadsvkgrftisrddarntvylqmnslkpedtavyyc
aaNPLDPNWTNSdywgqgtqgtvss#GGAGATATACCATGGAAGTTCAACTGGTTGAATCTGGTGGCGGTTTAGTTCAAGCCGGTGATAGCCTTCGTCTGAGCT
GCGCCGCAAGCGGAGATTACTGCATGGATTGCTTTATGGGTTGGTTTCGCCAGGCACCTGGTAAAGAACGTGAATTTGTGGCAGCCATTACCCCCTATTCCT
TTAAGACCTACTATGCCGATTCTGTTAAAGGTCGCTTCACTATTTCACGCGATGATGCGCGTAATACCGTGTACTTACAGATGAACTCACTGAAACCTGAGGA
TACCGCCGTTTATTATTGTGCGGCAAACCCTCTGGACCCCAACTGGACTAATTCTGACTATTGGGGTCAGGGCACACAAGGCACGGTCTCAAGCGGCGGAA
GCTCTGGTGAACAGAAACTGATTTCAGAAGCCAA
DYCMDCF#PYSFK#NPLDPNWTNS#evqlfesggglvqagdslrlscaasgDYCMDCFmgwfrqapgkerefvaaitPYSFKtyyadsvkgrftisrddarntvylqmnslkpedtavyyc
aaNPLDPNWTNSdywgqgtqgtvss#AGATATACCATGGAAGTTCAACTGTTTGAATCTGGTGGCGGTTTAGTTCAAGCCGGTGATAGCCTTCGTCTGAGCTGC
GCCGCAAGCGGAGATTACTGCATGGATTGCTTTATGGGTTGGTTTCGCCAGGCACCTGGTAAAGAACGTGAATTTGTGGCAGCCATTACCCCCTATTCCTTT
AAGACCTACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGGCAAACCCTCTGGACCCCAACTGGACTAATTCTGACTATTGGGGTCAGGGCACACAAGGCACGGTCTCAAGCGGCGGAAGC
TCTGGTGAACAGAAACTGATTTCAGAAGCCAA
DYCMDCF#PYSFK#SANILHLSVI#evqlvesggglvqagdslrlscaasgDYCMDCFmgwfrqapgkerefvaaitPYSFKtyyadsvkgrftisrddarntvylqmnslkpedtavyycaa
SANILHLSVInywgqgtqgtvss#ATATACCATGGAAGTTCAACTGGTTGAATCTGGTGGCGGTTTAGTTCAAGCCGGTGATAGCCTTCGTCTGAGCTGCGCCGCA
AGCGGAGATTACTGCATGGATTGCTTTATGGGTTGGTTTCGCCAGGCACCTGGTAAAGAACGTGAATTTGTGGCAGCCATTACCCCCTATTCCTTTAAGACCT
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTGCGGCAAGCGCGAACATTCTCCATTTGTCTGTTATCAACTATTGGGGTCAGGGCACACAAGGCACGGTCTCAAGCGGCGGAAGCTCTGGTGA
ACAGAAACTGATTTCAGAAGCCAA
DYCMDCF#PYSFK#WLTTQPFCGW#evqlvesggglvqagdslrlscaasgDYCMDCFmgwfrqapgkerefvaaitPYSFKtyyadsvrgrfsisadsakntvylqmnslkpedtavyy
caaWLTTQPFCGWnywgqgtqgtvss#ACCATGGAAGTTCAACTGGTTGAATCTGGTGGCGGTTTAGTTCAAGCCGGTGATAGCCTTCGTCTGAGCTGCGCCGC
AAGCGGAGATTACTGCATGGATTGCTTTATGGGTTGGTTTCGCCAGGCACCTGGTAAAGAACGTGAATTTGTGGCAGCCATTACCCCCTATTCCTTTAAGAC
CTACTATGCCGATTCTGTTCGTGGTCGCTTCAGCATTTCAGCAGATTCTGCGAAGAATACCGTGTACTTACAGATGAACTCACTGAAACCTGAGGATACCGCC
GTTTATTATTGTGCGGCATGGCTGACCACTCAGCCGTTCTGCGGTTGGAACTATTGGGGTCAGGGCACACAAGGCACGGTCTCAAGCGGCGGAAGCTCTGG
TGAACAGAAACTGATTTCAGAAGCCAA
DYCMDCF#PYSFK#PLNIGTATIV#evqlvesggglvqagdslrlscaasgDYCMDCFmgwfrqapgkerefvaaitPYSFKtyyadsvkgrftisrddarntvylqmnslkpedtavyycaa
PLNIGTATIVdywgqgtqvtvss#AAGGAGATATACCATGGAAGTTCAACTGGTTGAATCTGGTGGCGGTTTAGTTCAAGCCGGTGATAGCCTTCGTCTGAGCTG
CGCCGCAAGCGGAGATTACTGCATGGATTGCTTTATGGGTTGGTTTCGCCAGGCACCTGGTAAAGAACGTGAATTTGTGGCAGCCATTACCCCCTATTCCTT
TAAGACCTACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTGCGGCACCCCTCAATATCGGCACCGCGACGATTGTCGACTATTGGGGTCAGGGCACACAAGTCACGGTCTCAAGCGGCGGAAG
CTCTGGTGAACAGAAACTGATTTCAGAAGCCAA
DYCMDCF#PYSFK#PLNIGTATIA#evqlvdsggglvqagdslrlscaasgDYCMDCFmgwfrqapgkerefvaaitPYSFKtyyadsvkgrftisrddarntvylqmnslkpedtavyycaa
PLNIGTATIAdywgqgtqgtvss#GAAGGAGATATACCATGGAAGTTCAACTGGTTGACTCTGGTGGCGGTTTAGTTCAAGCCGGTGATAGCCTTCGTCTGAGCT
GCGCCGCAAGCGGAGATTACTGCATGGATTGCTTTATGGGTTGGTTTCGCCAGGCACCTGGTAAAGAACGTGAATTTGTGGCAGCCATTACCCCCTATTCCT
TTAAGACCTACTATGCCGATTCTGTTAAAGGTCGCTTCACTATTTCACGCGATGATGCGCGTAATACCGTGTACTTACAGATGAACTCACTGAAACCTGAGGA
TACCGCCGTTTATTATTGTGCGGCACCCCTCAATATCGGCACCGCGACGATTGCCGACTATTGGGGTCAGGGCACACAAGGCACGGTCTCAAGCGGCGGAA
GCTCTGGTGAACAGAAACTGATTTCAGAAGCCAA
DYCMDCF#PYSFK#NPLDPNWTNS#evqlvdsggglvqagdrlrlscaasgDYCMDCFmgwfrqapgkerefvaaitPYSFKtyyadsvkgrftisrddarntvylqmnslkpedtavyyc
aaNPLDPNWTNSdywgqgtqgtvss#GAAGGAGATATACCATGGAAGTTCAACTGGTTGACTCTGGTGGCGGTTTAGTTCAAGCCGGTGATCGCCTTCGTCTGA
GCTGCGCCGCAAGCGGAGATTACTGCATGGATTGCTTTATGGGTTGGTTTCGCCAGGCACCTGGTAAAGAACGTGAATTTGTGGCAGCCATTACCCCCTATT
CCTTTAAGACCTACTATGCCGATTCTGTTAAAGGTCGCTTCACTATTTCACGCGATGATGCGCGTAATACCGTGTACTTACAGATGAACTCACTGAAACCTGA
GGATACCGCCGTTTATTATTGTGCGGCAAACCCTCTGGACCCCAACTGGACTAATTCTGACTATTGGGGTCAGGGCACACAAGGCACGGTCTCAAGCGGCG
GAAGCTCTGGTGAACAGAAACTGATTTCAGAAGCCAA
DYCMDCF#PYSFK#LLPLDGFETS#evqlvesggglvqagdslrlscaasgDYCMDCFmgwfrqapgkerefvaaitPYSFKtyyadsvkgrftisrddarntvylqmnslkpedtavyycaa
LLPLDGFETSdywgqgtqgtvss#ATATACCATGGAAGTTCAACTGGTTGAATCTGGTGGCGGTTTAGTTCAAGCCGGTGATAGCCTTCGTCTGAGCTGCGCCGC
AAGCGGAGATTACTGCATGGATTGCTTTATGGGTTGGTTTCGCCAGGCACCTGGTAAAGAACGTGAATTTGTGGCAGCCATTACCCCCTATTCCTTTAAGAC
CTACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGCACTTCTCCCCCTGGATGGTTTTGAGACTTCGGACTATTGGGGTCAGGGCACACAAGGCACGGTCTCAAGCGGCGGAAGCTCTGGT
GAACAGAAACTGATTTCAGAAGCCAA
DYCMDCF#PYSFK#SMHLNNISIS#evqlvesggglvqagdslrlscaasgDYCMDCFmgwfrqapgkerefvaaitPYSFKtyyadsvkgrftisrddarntvylqmnslkpedtavyyca
aSMHLNNISISdywgqgtqgtvss#AAGGAGATATACCATGGAAGTTCAACTGGTTGAATCTGGTGGCGGTTTAGTTCAAGCCGGTGATAGCCTTCGTCTGAGCT
GCGCCGCAAGCGGAGATTACTGCATGGATTGCTTTATGGGTTGGTTTCGCCAGGCACCTGGTAAAGAACGTGAATTTGTGGCAGCCATTACCCCCTATTCCT
TTAAGACCTACTATGCCGATTCTGTTAAAGGTCGCTTCACTATTTCACGCGATGATGCGCGTAATACCGTGTACTTACAGATGAACTCACTGAAACCTGAGGA
TACCGCCGTTTATTATTGTGCGGCATCTATGCACCTGAACAATATCAGCATTAGTGACTATTGGGGTCAGGGCACACAAGGCACGGTCTCAAGCGGCGGAA
GCTCTGGTGAACAGAAACTGATTTCAGAAGCCAA
DYCMDCF#PYSFK#PFAPLTVLLA#evqlvesggglvqagdslrlscaasgDYCMDCFmgwfrqapgkerefvaaitPYSFKtyyadsvkgrftisrddarntvylqmnslkpedtavyyca
aPFAPLTVLLAeywgqgtqgtvss#AGGAGATATACCATGGAAGTTCAACTGGTTGAATCTGGTGGCGGTTTAGTTCAAGCCGGTGATAGCCTTCGTCTGAGCTG
CGCCGCAAGCGGAGATTACTGCATGGATTGCTTTATGGGTTGGTTTCGCCAGGCACCTGGTAAAGAACGTGAATTTGTGGCAGCCATTACCCCCTATTCCTT
TAAGACCTACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTGCGGCACCGTTCGCGCCCCTTACCGTTCTTCTCGCGGAGTATTGGGGTCAGGGCACACAAGGCACGGTCTCAAGCGGCGGAAG
CTCTGGTGAACAGAAACTGATTTCAGAAGCCAA
DYCMDCF#PYSFK#NPLDPNWTNS#evqlvesggglvqagdslrlscaasgDYCMDCFmgwfrqapgkerefvaaitPYSFKtyyadsvkgrftisrddacntvylqmnslkpedtavyy
caaNPLDPNWTNSdywgqgtqvtvss#GAAGGAGATATACCATGGAAGTTCAACTGGTTGAATCTGGTGGCGGTTTAGTTCAAGCCGGTGATAGCCTTCGTCTG
AGCTGCGCCGCAAGCGGAGATTACTGCATGGATTGCTTTATGGGTTGGTTTCGCCAGGCACCTGGTAAAGAACGTGAATTTGTGGCAGCCATTACCCCCTAT
TCCTTTAAGACCTACTATGCCGATTCTGTTAAAGGTCGCTTCACTATTTCACGCGATGATGCGTGTAATACCGTGTACTTACAGATGAACTCACTGAAACCTG
AGGATACCGCCGTTTATTATTGTGCGGCAAACCCTCTGGACCCCAACTGGACTAATTCTGACTATTGGGGTCAGGGCACACAAGTCACGGTCTCAAGCGGCG
GAAGCTCTGGTGAACAGAAACTGATTTCAGAAGCCAA
DYCMDCF#PYSFK#QVSLVTHGDL#evqlvesggglvqagdslrlscatsgDYCMDCFmgwfrqapgkerefvaaitPYSFKtyyadsvkgrftisrddarntvylqmnslkpedtavyyca
aQVSLVTHGDLdywgqgtqvtvss#ATATACCATGGAAGTTCAACTGGTTGAATCTGGTGGCGGTTTAGTTCAAGCCGGTGATAGCCTTCGTCTGAGCTGCGCC
ACAAGCGGAGATTACTGCATGGATTGCTTTATGGGTTGGTTTCGCCAGGCACCTGGTAAAGAACGTGAATTTGTGGCAGCCATTACCCCCTATTCCTTTAAG
ACCTACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTGCGGCACAGGTCTCCCTTGTTACCCATGGGGATCTGGACTATTGGGGTCAGGGCACACAAGTCACGGTCTCAAGCGGCGGAAGCTCTG
GTGAACAGAAACTGATTTCAGAAGCCAA
DYCMDCF#PYSFK#DSLILANSQW#evqlvesggglvqagdslrlscaasgDYCMDCFmdwfrqapgkerefvaaitPYSFKtyyadsvkgrftisrddarntvylqmnslkpedtavyyc
aaDSLILANSQWdyqgqgtqgtvss#ATACCATGGAAGTTCAACTGGTTGAATCTGGTGGCGGTTTAGTTCAAGCCGGTGATAGCCTTCGTCTGAGCTGCGCCGC
AAGCGGAGATTACTGCATGGATTGCTTTATGGATTGGTTTCGCCAGGCACCTGGTAAAGAACGTGAATTTGTGGCAGCCATTACCCCCTATTCCTTTAAGAC
CTACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGCAGACAGCCTTATTTTGGCTAATTCTCAGTGGGACTATTGGGGTCAGGGCACACAAGGCACGGTCTCAAGCGGCGGAAGCTCTGG
TGAACAGAAACTGATTTCAGAAGCCAA
DYCMDCF#PYSFK#YTSHSDTAFL#evqlvesggglvqagdslrlscaasgDYCMDCFmgwfrqapgkerefvaaitPYSFKtyyadsvkgrftisrddarntvylqmnslkpedtavyyca
aYTSHSDTAFLnywgqgtqvtvss#ACCATGGAAGTTCAACTGGTTGAATCTGGTGGCGGTTTAGTTCAAGCCGGTGATAGCCTTCGTCTGAGCTGCGCCGCAAG
CGGAGATTACTGCATGGATTGCTTTATGGGTTGGTTTCGCCAGGCACCTGGTAAAGAACGTGAATTTGTGGCAGCCATTACCCCCTATTCCTTTAAGACCTAC
TATGCCGATTCTGTTAAAGGTCGCTTCACTATTTCACGCGATGATGCGCGTAATACCGTGTACTTACAGATGAACTCACTGAAACCTGAGGATACCGCCGTTT
ATTATTGTGCGGCATACACCAGCCACTCGGATACGGCTTTTCTGAACTATTGGGGTCAGGGCACACAAGTCACGGTCTCAAGCGGCGGAAGCTCTGGTGAA
CAGAAACTGATTTCAGAAGCCAA
DYCMDCF#PYSFK#PLNIGTATIA#evqlfesggglvqagdslrlscaasgDYCMDCFmgwfrqapgkerefvaaitPYSFKtyyadsvkgrftisrddarntvylqmnslkpedtavyycaa
PLNIGTATIAdywgqgtqgtvss#AGATATACCATGGAAGTTCAACTGTTTGAATCTGGTGGCGGTTTAGTTCAAGCCGGTGATAGCCTTCGTCTGAGCTGCGCC
GCAAGCGGAGATTACTGCATGGATTGCTTTATGGGTTGGTTTCGCCAGGCACCTGGTAAAGAACGTGAATTTGTGGCAGCCATTACCCCCTATTCCTTTAAG
ACCTACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTGCGGCACCCCTCAATATCGGCACCGCGACGATTGCCGACTATTGGGGTCAGGGCACACAAGGCACGGTCTCAAGCGGCGGAAGCTCT
GGTGAAAAGAAACTGATTTCAGAAGCCAA
DYCMDCF#PYSFK#SANILHLSVI#evqlvesggglvqagdslrlscaasgDYCMDCFmgwfrqapgkerefvaaitPYSFKtyyadsvkgrftisrddvrntvylqmnslkpedtavyycaa
SANILHLSVInywgqgtqvtvss#GGAGATATACCATGGAAGTTCAACTGGTTGAATCTGGTGGCGGTTTAGTTCAAGCCGGTGATAGCCTTCGTCTGAGCTGCG
CCGCAAGCGGAGATTACTGCATGGATTGCTTTATGGGTTGGTTTCGCCAGGCACCTGGTAAAGAACGTGAATTTGTGGCAGCCATTACCCCCTATTCCTTTA
AGACCTACTATGCCGATTCTGTTAAAGGTCGCTTCACTATTTCACGCGATGATGTGCGTAATACCGTGTACTTACAGATGAACTCACTGAAACCTGAGGATAC
CGCCGTTTATTATTGTGCGGCAAGCGCGAACATTCTCCATTTGTCTGTTATCAACTATTGGGGTCAGGGCACACAAGTCACGGTCTCAAGCGGCGGAAGCTC
TGGTGAACAGAAACTGATTTCAGAAGCCAA
DYCMDCF#PYSFK#PFAPLTVLLA#evqlvesggglvqagdslrlscaasgDYCMDCFmgwfrqapgkerefvaaitPYSFKtyyadsvkgrftisrddarntvylqmnslkpedtavyyca
aPFAPLTVLLAeywvqgtqgtvss#ACCATGGAAGTTCAACTGGTTGAATCTGGTGGCGGTTTAGTTCAAGCCGGTGATAGCCTTCGTCTGAGCTGCGCCGCAAG
CGGAGATTACTGCATGGATTGCTTTATGGGTTGGTTTCGCCAGGCACCTGGTAAAGAACGTGAATTTGTGGCAGCCATTACCCCCTATTCCTTTAAGACCTAC
TATGCCGATTCTGTTAAAGGTCGCTTCACTATTTCACGCGATGATGCGCGTAATACCGTGTACTTACAGATGAACTCACTGAAACCTGAGGATACCGCCGTTT
ATTATTGTGCGGCACCGTTCGCGCCCCTTACCGTTCTTCTCGCGGAGTATTGGGTTCAGGGCACACAAGGCACGGTCTCAAGCGGCGGAAGCTCTGGTGAA
AGGAAAATGATTTCAGAAGCCAA

TABLE 6
Sequences belonging to cluster SR12. Each line in the table represents one sequence,
both segments and full length of the sequence are shown. Shown items are divided by “#” and in
the order from start to end of each line is: CDR1 amino acid sequence, CDR2 amino acid sequence,
CDR3 amino acid sequence, full-length amino acid sequence with CDRs capitalized, full-length
DNA sequence.
(SEQ ID NOS: 2031-2755)

AIVHVFA#LDNDA#FCYVPLATC#evqlvesggglvqagdslrlscaasgAIVHVFAmgwfrqapgkerefvasinLDNDAtnyadsvkgrftisrddarntvylqmnslkpedtavyycna
FCYVPLATCdywgqgtqvtvss#ATACCATGGAAGTTCAACTGGTTGAATCTGGTGGCGGTTTAGTTCAAGCCGGTGATAGCCTTCGTCTGAGCTGCGCCGCAAG
CGGAGCCATTGTGCATGTGTTCGCTATGGGTTGGTTTCGCCAGGCACCTGGTAAAGAACGTGAATTTGTGGCATCCATTAACCTTGACAACGACGCTACCAA
CTATGCCGATTCTGTTAAAGGTCGCTTCACTATTTCACGCGATGATGCGCGTAATACCGTGTACTTACAGATGAACTCACTGAAACCTGAGGATACCGCCGTT
TATTATTGTAATGCATTCTGCTACGTCCCTTTGGCCACGTGTGACTATTGGGGTCAGGGCACACAAGTCACGGTCTCAAGCGGCGTAAGCTCTGGTGAACAG
AAACTGATTTCAGAAG
AIVHVFA#LDNDA#PFWINSSVV#evqlvesggglvqagdslrlscaasgAIVHVFAmgwfrqapgkerefvasinLDNDAtnyadsvkgrftisrddahntvylqmnslkpedtavyycn
aPFWINSSVVdywgqgtqvtvss#ATATACCATGGAAGTTCAACTGGTTGAATCTGGTGGCGGTTTAGTTCAAGCCGGTGATAGCCTTCGTCTGAGCTGCGCCGC
AAGCGGAGCCATTGTGCATGTGTTCGCTATGGGTTGGTTTCGCCAGGCACCTGGTAAAGAACGTGAATTTGTGGCATCCATTAACCTTGACAACGACGCTAC
CAACTATGCCGATTCTGTTAAAGGTCGCTTCACTATTTCACGCGATGATGCGCATAATACCGTGTACTTACAGATGAACTCACTGAAACCTGAGGATACCGCC
GTTTATTATTGTAATGCACCCTTCTGGATCAATAGTTCTGTTGTGGACTATTGGGGTCAGGGCACACAAGTCACGGTCTCAAGCGGCGGAAGCTCTGGTGAA
CAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdywgqgtqvtvss#AGGAGATATACCATGGAAGTTCAACTGGTTGAATCTGGTGGCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTAATGCAACCATCTTTATGGATTCCGATTTGGGTGACTATTGGGGTCAGGGCACACAAGTCACGGTCTCAAGCGGCGGAAGCTCTG
GTGAACAGAAACTGATTTCAGAAG
AIVHVFA#LDNDA#PFWINSSVV#evqliesggglvqagdslrlscaasgAIVHVFAmgwfrqapgkerefvasinLDNDAtnyadsvkgrftisrddahntvylqmnslkpedtavyycna
PFWINSSVVdywgqgtqvtvss#AGGAGATATACCATGGAAGTTCAACTGATTGAATCTGGTGGCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGCGCATAATACCGTGTACTTACAGATGAACTCACTGAAACCTGAGGATA
CCGCCGTTTATTATTGTAATGCACCCTTCTGGATCAATAGTTCTGTTGTGGACTATTGGGGTCAGGGCACACAAGTCACGGTCTCAAGCGGCGGAAGCTCTG
GTGAACAGAAACTGATTTCAGAAG
AIVHVFA#LDNDA#PFWINSSVV#qvqlfesggglvqagdslrlscaasgAIVHVFAmgwfrqapgkerefvasinLDNDAtnyadsvkgrftisrddahntvylqmnslkpedtavyycn
aPFWINSSVVdywgqgtqvtvss#ATATACCATGCAGGTTCAACTGTTTGAATCTGGTGGCGGTTTAGTTCAAGCCGGTGATAGCCTTCGTCTGAGCTGCGCCGC
AAGCGGAGCCATTGTGCATGTGTTCGCTATGGGTTGGTTTCGCCAGGCACCTGGTAAAGAACGTGAATTTGTGGCATCCATTAACCTTGACAACGACGCTAC
CAACTATGCCGATTCTGTTAAAGGTCGCTTCACTATTTCACGCGATGATGCGCATAATACCGTGTACTTACAGATGAACTCACTGAAACCTGAGGATACCGCC
GTTTATTATTGTAATGCACCCTTCTGGATCAATAGTTCTGTTGTGGACTATTGGGGTCAGGGCACACAAGTCACGGTCTCAAGCGGCGGAAGCTCTGGTGAA
CAGAAACTGATTTCAGAAG
AIVHVFA#LDNDA#TIFMDSDLG#evqlfesggglvqagdslrlscaasgAIVHVFAmgwfrqapgkerefvasinLDNDAtnyadsvkgrftisrddarntvylqmnslkpedtavyycna
TIFMDSDLGdywgqgtqvtvss#AGATATACCATGGAAGTTCAACTGTTTGAATCTGGTGGCGGTTTAGTTCAAGCCGGTGATAGCCTTCGTCTGAGCTGCGCCG
CAAGCGGAGCCATTGTGCATGTGTTCGCTATGGGTTGGTTTCGCCAGGCACCTGGTAAAGAACGTGAATTTGTGGCATCCATTAACCTTGACAACGACGCTA
CCAACTATGCCGATTCTGTTAAAGGTCGCTTCACTATTTCACGCGATGATGCGCGTAATACCGTGTACTTACAGATGAACTCACTGAAACCTGAGGATACCGC
CGTTTATTATTGTAATGCAACCATCTTTATGGATTCCGATTTGGGTGACTATTGGGGTCAGGGCACACAAGTCACGGTCTCAAGCGGCGGAAGCTCTGGTGA
ACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGaywgqgtqvtvss#AAGGAGATATACCATGGAAGTTCAACTGGTTGAATCTGGTGGCGGTTTAGTTCAAGCCGGTGATAGCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTAATGCAACCATCTTTATGGATTCCGATTTGGGTGCCTATTGGGGTCAGGGCACACAAGTCACGGTCTCAAGCGGCGGAAGCTCT
GGTGAACAGAAACTGATTTCAGAAG
AIVHVFA#LDNDA#TAISPISIVD#evqlvesggglvqagdslrlscaasgAIVHVFAmgwfrqapgkerefvasinLDNDAtnyadsvkgrftisrddarntvylqmnslkpedtavyycaa
TAISPISIVDeywgqgtqvtvss#ATACCATGGAAGTTCAACTGGTTGAATCTGGTGGCGGTTTAGTTCAAGCCGGTGATAGCCTTCGTCTGAGCTGCGCCGCAAG
CGGAGCCATTGTGCATGTGTTCGCTATGGGTTGGTTTCGCCAGGCACCTGGTAAAGAACGTGAATTTGTGGCATCCATTAACCTTGACAACGACGCTACCAA
CTATGCCGATTCTGTTAAAGGTCGCTTCACTATTTCACGCGATGATGCGCGTAATACCGTGTACTTACAGATGAACTCACTGAAACCTGAGGATACCGCCGTT
TATTATTGTGCGGCAACGGCTATTAGCCCTATCTCTATTGTGGATGAGTATTGGGGTCAGGGCACACAAGTCACGGTCTCAAGCGGCGGAAGCTCTGGTGA
ACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpvdtavyycna
TIFMDSDLGdywgqgtqvtvss#ATATACCATGGAAGTTCAACTGGTTGAATCTGGTGGCGGTTTAGTTCAAGCCGGTGATAGCCTTCGTCTGAGCTGCGCCGC
AAGCGGAGCCATTGTGCATGTGTTCGCTATGGGTTGGTTTCGCCAGGCACCTGGTAAAGAACGTGAATTTGTGGCATCCATTAACCTTGACAACGACGCTAC
CAACTATGCCGATTCTGTTAAAGGTCGCTTCACTATTTCACGCGATGATGCGCGTAATACCGTGTACTTACAGATGAACTCACTGAAACCTGTGGATACCGCC
GTTTATTATTGTAATGCAACCATCTTTATGGATTCCGATTTGGGTGACTATTGGGGTCAGGGCACACAAGTCACGGTCTCAAGCGGCGGAAGCTCTGGTGAA
CAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGaywgqdtqvtvss#ACCATGGAAGTTCAACTGGTTGAATCTGGTGGCGGTTTAGTTCAAGCCGGTGATAGCCTTCGTCTGAGCTGCGCCGCAAGC
GGAGCCATTGTGCATGTGTTCGCTATGGGTTGGTTTCGCCAGGCACCTGGTAAAGAACGTGAATTTGTGGCATCCATTAACCTTGACAACGACGCTACCAAC
TATGCCGATTCTGTTAAAGGTCGCTTCACTATTTCACGCGATGATGCGCGTAATACCGTGTACTTACAGATGAACTCGCTGAAACCTGAGGATACCGCCGTTT
ATTATTGTAATGCAACCATCTTTATGGATTCCGATTTGGGTGCCTATTGGGGTCAGGACACACAAGTCACGGTCTCAAGCGGCGGAAGCTCTGGTGAACAGA
AACTGATTTCAGAAG
AIVHVFA#LDNDA#FCYVPLATC#evqlvesggglvqagdslrlscaasgAIVHVFAmgwfrqapgkerefvasinLDNDAtnyadsvkgrftisrddarntvylqmnslkpedtavyycna
FCYVPLATCdylgqgtqvtvss#GGAGATATACCATGGAAGTTCAACTGGTTGAATCTGGTGGCGGTTTAGTTCAAGCCGGTGATAGCCTTCGTCTGAGCTGCGC
CGCAAGCGGAGCCATTGTGCATGTGTTCGCTATGGGTTGGTTTCGCCAGGCACCTGGTAAAGAACGTGAATTTGTGGCATCCATTAACCTTGACAACGACGC
TACCAACTATGCCGATTCTGTTAAAGGTCGCTTCACTATTTCACGCGATGATGCGCGTAATACCGTGTACTTACAGATGAACTCACTGAAACCTGAGGATACC
GCCGTTTATTACTGTAATGCATTCTGCTACGTCCCTTTGGCCACGTGTGACTATTTGGGTCAGGGCACACAAGTCACGGTCTCAAGCGGCGGAAGCTCTGGT
GAACAGAAACTGATTTCAGAAG
AIVHVFA#LDSDA#TAISPISIVD#evqlvesggglvqagdslrlscaasgAIVHVFAmgwfrqapgkerefvasinLDSDAtnyadsvkgrftisrddarntvylqmnslkpedtavyycaaT
AISPISIVDeywgqgtqvtvss#AAGGAGATATACCATGGAAGTTCAACTGGTTGAATCTGGTGGCGGTTTAGTTCAAGCCGGTGATAGCCTTCGTCTGAGCTGCG
CCGCAAGCGGAGCCATTGTGCATGTGTTCGCTATGGGTTGGTTTCGCCAGGCACCTGGTAAAGAACGTGAATTTGTGGCATCCATTAACCTTGACAGCGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGGCAACGGCTATTAGCCCTATCTCTATTGTGGATGAGTATTGGGGTCAGGGCACACAAGTCACGGTCTCAAGCGGCGGAAGCT
CTGGTGAACAGAAACTGATTTCAGAAG
AIVHVFA#LDNDA#PFWINSSVV#evqlveyggglvqagdslrlscaasgAIVHVFAmgwfrqapgkerefvasinLDNDAtnyadsvkgrftisrddarntvylqmnslkpedtavyycn
aPFWINSSVVdywgqgtqvtvss#GGAGATATACCATGGAAGTTCAACTGGTTGAATATGGTGGCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTAATGCACCCTTCTGGATCAATAGTTCTGTTGTGGACTATTGGGGTCAGGGCACACAAGTCACGGTCTCAAGCGGCGGAAGCTCTG
GTGAACAGAAACTGATTTCAGAAG
AIVHVFA#LDNDA#TIFMDSDLG#qvqlvesggglvqagdslrlscaasgAIVHVFAmgwfrqapgkerefvasinLDNDAtnyadsvkgrftisrddarntvylqmnslkpedtavyycn
aTIFMDSDLGdywgqgtqvtvss#GGAGATATACCATGCAGGTTCAACTGGTTGAATCTGGTGGCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTAATGCAACCATCTTTATGGATTCCGATTTGGGTGACTATTGGGGTCAGGGCACACAAGTCACGGTCTCAAGCGGCGGAAGCTCTG
GTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddvrntvylqmnslkpedtavyycna
TIFMDSDLGdywgqgtqvtvss#AGGAGATATACCATGGAAGTTCAACTGGTTGAATCTGGTGGCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGTGCGTAATACCGTGTACTTACAGATGAACTCACTGAAACCTGAGGATA
CCGCCGTTTATTATTGTAATGCAACCATCTTTATGGATTCCGATTTGGGTGACTATTGGGGTCAGGGCACACAAGTCACGGTCTCAAGCGGCGGAAGCTCTG
GTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkglftisrddarntvylqmnslkpedtavyycna
TIFMDSDLGdywgqgtqvtvss#GAAGGAGATATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCTCTTCACTATTTCACGCGATGATGCGCGTAATACCGTGTACTTACAGATGAACTCACTGAAACCTGAGG
ATACCGCCGTTTATTATTGTAATGCAACCATCTTTATGGATTCCGATTTGGGTGACTATTGGGGTCAGGGCACACAAGTCACGGTCTCAAGCGGCGGAAGCT
CTGGTGAACAGAAACTGATTTCAGAAG
AIVHVFT#LDNDA#FCYVPLATC#evqlvesggglvqagdslrlscaasgAIVHVFTmgwfrqapgkerefvasinLDNDAtnyadsvkgrftisrddarntvylqmnslkpedtavyycna
GCYVPLATCdywgqgtqdtvss#ATACCATGGAAGTTCAACTGGTTGAATCTGGTGGCGGTTAGTTCAAGCCGGTGATAGCCTTCGTCTGAGCTGCGCCGCAA
GCGGAGCCATTGTGCATGTGTTCACTATGGGTTGGTTTCGCCAGGCACCTGGTAAAGAACGTGAATTTGTGGCATCCATTAACCTTGACAACGACGCTACCA
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTAATGCATTCTGCTACGTCCCTTTGGCCACGTGTGACTATTGGGGTCAGGGCACACAAGACACGGTCTCAAGCGGCGGAAGCTCTGGTGAACA
GAAACTGATTTCAGAAG
AIVHVFA#LDNDA#TAISPISIVD#evqlvesggglvqagdslrlscaasgAIVHVFAmgwfrqapgkerefvasinLDNDAtnyadsvkgrftisrddarntvylqmnslkpedtavyccaa
TAISPISIVDeywgqgtqvtvss#ATACCATGGAAGTTCAACTGGTTGAATCTGGTGGCGGTTTAGTTCAAGCCGGTGATAGCCTTCGTCTGAGCTGCGCCGCAAG
CGGAGCCATTGTGCATGTGTTCGCTATGGGTTGGTTTCGCCAGGCACCTGGTAAAGAACGTGAATTTGTGGCATCCATTAACCTTGACAACGACGCTACCAA
CTATGCCGATTCTGTTAAGGGTCGCTTCACTATTTCACGCGATGATGCGCGTAATACCGTGTACTTACAGATGAACTCACTGAAACCTGAGGATACCGCCGTT
TATTGTTGTGCGGCAACGGCTATTAGCCCTATCTCTATTGTGGATGAGTATTGGGGTCAGGGCACACAAGTCACGGTCTCAAGCGGCGGAAGCTCTGGTGA
ACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarytvylqmnslkpedtavyycna
TIFMDSDLGdywgqgtqvtvss#GAAGGAGATATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTTATACCGTGTACTTACAGATGAACTCACTGAAACCTGAGG
ATACCGCCGTTTATTATTGTAATGCAACCATCTTTATGGATTCCGATTTGGGTGACTATTGGGGTCAGGGCACACAAGTCACGGTCTCAAGCGGCGGAAGCT
CTGGTGAACAGAAACTGATTTCAGAAG
AIVHVFA#LDNDA#TIFMDSDLG#kvqlvesggglvqagdslrlscaasgAIVHVFAmgwfrqapgkerefvasinLDNDAtnyadsvkgrftisrddarntvylqmnslkpedtavyycna
TIFMDSDLGdywgqgtqvtvss#GAAGGAGATATACCATGAAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTAATGCAACCATCTTTATGGATTCCGATTTGGGTGACTATTGGGGTCAGGGCACACAAGTCACGGTCTCAAGCGGCGGAAGC
TCTGGTGAACAGAAACTGATTTCAGAAG
AIVHVFA#LDNDA#TIFMDSDLG#evqlvesggglvqagdslrpscaasgAIVHVFAmgwfrqapgkerefvasinLDNDAtnytdsvkgrftisrddarntvylqmnslkpedtavyycn
aTIFMDSDLGdywgqgtqvtvss#GGAGATATACCATGGAAGTTCAACTGGTTGAATCTGGTGGCGGTTTAGTTCAAGCCGGTGATAGCCTTCGTCCGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATACCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTAATGCAACCATCTTTATGGATTCCGATTTGGGTGACTATTGGGGTCAGGGCACACAAGTCACGGTCTCAAGCGGCGGAAGCTCTG
GTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdywgqgpqvtvss#AAGGAGATATACCATGGAAGTTCAACTGGTTGAATCTGGTGGCGGTTTAGTTCAAGCCGGTGATAGCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTAATGCAACCATCTTTATGGATTCCGATTTGGGTGACTATTGGGGTCAGGGCCCACAAGTCACGGTCTCAAGCGGCGGAAGCTCT
GGTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrydarntvylqmnslkpedtavyycna
TIFMDSDLGdywgqgtqvtvss#AGATATACCATGGAAGTTCAACTGGTTGAATCTGGTGGCGGTTTAGTTCAAGCCGGTGATAGCCTTCGTCTGAGCTGCGCC
GCAAGCGGAGCCATTGTGCATGTGTTCGCTATGGGTTGGTTTCGCCAGGCACCTGGTAAAGAACGTGAATTTGTGGCATCCATTAACCTTGACAACGACGCT
ACCAACTATGCCGATTCTGTTAAAGGTCGCTTCACTATTTCACGCTATGATGCGCGTAATACCGTGTACTTACAGATGAACTCACTGAAACCTGAGGATACCG
CCGTTTATTATTGTAATGCAACCATCTTTATGGATTCCGATTTGGGTGACTATTGGGGTCAGGGCACACAAGTCACGGTCTCAAGCGGCGGAAGCTCTGGTG
AACAGAAACTGATTTCAGAAG
AIVHVFA#LDNDA#TAISPISIVD#evqlvesggglvqagdslrlscaasgAIVHVFAmgwfrqapgkerefvasinLDNDAtnyadsvkgrftisrddarntvylqmnslkpedtavyycva
TAISPISIVDeywgqgtqvtvss#GGAGATATACCATGGAAGTTCAACTGGTTGAATCTGGTGGCGGTTTAGTTCAAGCCGGTGATAGCCTTCGTCTGAGCTGCGC
CGCAAGCGGAGCCATTGTGCATGTGTTCGCTATGGGTTGGTTTCGCCAGGCACCTGGTAAAGAACGTGAATTTGTGGCATCCATTAACCTTGACAACGACGC
TACCAACTATGCCGATTCTGTTAAAGGTCGCTTCACTATTTCACGCGATGATGCGCGTAATACCGTGTACTTACAGATGAACTCACTGAAACCTGAGGATACC
GCCGTTTATTATTGTGTGGCAACGGCTATTAGCCCTATCTCTATTGTGGATGAGTATTGGGGTCAGGGCACACAAGTCACGGTCTCAAGCGGCGGAAGCTCT
GGTGAACAGAAACTGATTTCAGAAG
AIVHVFA#LDNDA#TIFMDSDLG#evqlvesggglvqagdslrqscaasgAIVHVFAmgwfrqapgkerefvasinLDNDAtnyadsvkgrftisrddarntvylqmnslkpedtavyycn
aTIFMDSDLGdywgqgtqvtvss#GGAGATATACCATGGAAGTTCAACTGGTTGAATCTGGTGGCGGTTTAGTTCAAGCCGGTGATAGCCTTCGTCAGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTAATGCAACCATCTTTATGGATTCCGATTTGGGTGACTATTGGGGTCAGGGCACACAAGTCACGGTCTCAAGCGGCGGAAGCTCTG
GTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntmylqmnslkpedtavyycn
aTIFMDSDLGdywgqgtqvtvss#ATATACCATGGAAGTTCAACTGGTTGAATCTGGTGGCGGTTTAGTTCAAGCCGGTGATAGCCTTCGTCTGAGCTGCGCCGC
AAGCGGAGCCATTGTGCATGTGTTCGCTATGGGTTGGTTTCGCCAGGCACCTGGTAAAGAACGTGAATTTGTGGCATCCATTAACCTTGACAACGACGCTAC
CAACTATGCCGATTCTGTTAAAGGTCGCTTCACTATTTCACGCGATGATGCGCGTAATACCATGTACTTACAGATGAACTCACTGAAACCTGAGGATACCGCC
GTTTATTATTGTAATGCAACCATCTTTATGGATTCCGATTTGGGTGACTATTGGGGTCAGGGCACACAAGTCACGGTCTCAAGCGGCGGAAGCTCTGGTGAA
CAGAAACTGATTTCAGAAG
AIVHVFA#LDNDA#ACSDYTYGSHGVR#evqlvesggglvqagdslrlscaasgAIVHVFAmgwfrqapgkerefvasinLDNDAtnyadsvkgrftisrddarntvylqmnslkpedtav
yycnaACSDYTYGSHGVRdswgqgtqvtvss#AGGAGATATACCATGGAAGTTCAACTGGTTGAATCTGGTGGCGGTTTAGTTCAAGCCGGTGATAGCCTTCGTC
TGAGCTGCGCCGCAAGCGGAGCCATTGTGCATGTGTTCGCTATGGGTTGGTTTCGCCAGGCACCTGGTAAAGAACGTGAATTTGTGGCATCCATTAACCTTG
ACAACGACGCTACCAACTATGCCGATTCTGTTAAAGGTCGCTTCACTATTTCACGCGATGATGCGCGTAATACCGTGTACTTACAGATGAACTCACTGAAACC
TGAGGATACCGCCGTTTATTATTGTAATGCAGCGTGTTCGGACTATACGTATGGCTCTCATGGTGTTCGGGACTCTTGGGGTCAGGGCACACAAGTCACGGT
CTCAAGCGGCGGAAGCTCTGGTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdywgqgkqvtvss#ACCATGGAAGTTCAACTGGTTGAATCTGGTGGCGGTTTAGTTCAAGCCGGTGATAGCCTTCGTCTGAGCTGCGCCGCAAGC
GGAGCCATTGTGCATGTGTTCGCTATGGGTTGGTTTCGCCAGGCACCTGGTAAAGAACGTGAATTTGTGGCATCCATTAACCTTGACAACGACGCTACCAAC
TATGCCGATTCTGTTAAAGGTCGCTTCACTATTTCACGCGATGATGCGCGTAATACCGTGTACTTACAGATGAACTCACTGAAACCTGAGGATACCGCCGTTT
ATTATTGTAATGCAACCATCTTTATGGATTCCGATTTGGGTGACTATTGGGGTCAGGGCAAACAAGTCACGGTCTCAAGCGGCGGAAGCTCTGGTGAACAG
AAACTGATTTCAGAAG
AIVHVFA#LDNDA#WSFTPSICTL#evqlvesggglvqagdslrlscaasgAIVHVFAmgwfrqapgkerefvasinLDNDAtnyadsvkgrftisrddarntvylqmnslkpedtavyyca
aWSFTPSICTLeywgqgtqvtvss#ACCATGGAAGTTCAACTGGTTGAATCTGGTGGCGGTTTAGTTCAAGCCGGTGATAGCCTTCGTCTGAGCTGCGCCGCAAG
CGGAGCCATTGTGCATGTGTTCGCTATGGGTTGGTTTCGCCAGGCACCTGGTAAAGAACGTGAATTTGTGGCATCCATTAACCTTGACAACGACGCTACCAA
CTATGCCGATTCTGTTAAAGGTCGCTTCACTATTTCACGCGATGATGCGCGTAATACCGTGTACTTACAGATGAACTCACTGAAACCTGAGGATACCGCCGTT
TATTATTGTGCGGCATGGAGTTTCACCCCTTCTATCTGTACCTTGGAGTATTGGGGTCAGGGCACACAAGTCACGGTCTCAAGCGGCGGAAGCTCTGGTGAA
CAGAAACTGATTTCAGAAG
AIVHVFA#LDNDA#TIFMDSVLG#evqlvesggglvqagdslrlscaasgAIVHVFAmgwfrqapgkerefvasinLDNDAtnyadsvkgrftisrddarntvylqmnslkpedtavyycna
TIFMDSVLGdywgqgtqvtvss#ACCATGGAAGTTCAACTGGTTGAATCTGGTGGCGGTTTAGTTCAAGCCGGTGATAGCCTTCGTCTGAGCTGCGCCGCAAGC
GGAGCCATTGTGCATGTGTTCGCTATGGGTTGGTTTCGCCAGGCACCTGGTAAAGAACGTGAATTTGTGGCATCCATTAACCTTGACAACGACGCTACCAAC
TATGCCGATTCTGTTAAAGGTCGCTTCACTATTTCACGCGATGATGCGCGTAATACCGTGTACTTACAGATGAACTCACTGAAACCTGAGGATACCGCCGTTT
ATTATTGTAATGCAACCATCTTTATGGATTCCGTTTTGGGTGACTATTGGGGTCAGGGCACACAAGTCACGGTCTCAAGCGGCGGAAGCTCTGGTGAACAGA
AACTGATTTCAGAAG
AIVHVFA#LDNDA#PFWINSSVV#evqlvesggglvqagdslrlscaasgAIVHVFAmgwfrqapgkerefvasinLDNDAtnyadsvkgrftisrddahntvylqmnslkpedtavyycn
aPFWINSSVVdylgqgtqvtvss#AGATATACCATGGAAGTTCAACTGGTTGAATCTGGTGGCGGTTTAGTTCAAGCCGGTGATAGCCTTCGTCTGAGCTGCGCC
GCAAGCGGAGCCATTGTGCATGTGTTCGCTATGGGTTGGTTTCGCCAGGCACCTGGTAAAGAACGTGAATTTGTGGCATCCATTAACCTTGACAACGACGCT
ACCAACTATGCCGATTCTGTTAAAGGTCGCTTCACTATTTCACGCGATGATGCGCATAATACCGTGTACTTACAGATGAACTCACTGAAACCTGAGGATACCG
CCGTTTATTATTGTAATGCACCCTTCTGGATCAATAGTTCTGTTGTGGACTATTTGGGTCAGGGCACACAAGTCACGGTCTCAAGCGGCGGAAGCTCTGGTG
AACAGAAACTGATTTCAGAAG
AIVHVFA#LDNDA#PFWINSSVV#evqlvesggglvqagdslrlscaasgAIVHVFAmgwfrqapgkerefvasinLDNDAtnyadsvkgrftisrddahntvylqmnslkpedtavyycn
aPFWINSSVVdywgqgtqvtvsr#AAGGAGATATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCATAATACCGTGTACTTACAGATGAACTCACTGAAACCTGAGG
ATACCGCCGTTTATTATTGTAATGCACCCTTCTGGATCAATAGTTCTGTTGTGGACTATTGGGGTCAGGGCACACAAGTCACGGTCTCACGCGGCGGAAGCT
CTGGTGAACAGAAACTGATTTCAGAAG
AIVHVFA#LDNDA#TIFMDSDLG#evqlvesgvglvqagdslrlscaasgAIVHVFAmgwfrqapgkerefvasinLDNDAtnyadsvkgrftisrddarntvylqmnslkpedtavyycna
TIFMDSDLGdywgqgtqvtvss#AGGAGATATACCATGGAAGTTCAACTGGTTGAATCTGGTGTCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTAATGCAACCATCTTTATGGATTCCGATTTGGGTGACTATTGGGGTCAGGGCACACAAGTCACGGTCTCAAGCGGCGGAAGCTCTG
GTGAACAGAAACTGATTTCAGAAG
AIVHVFA#LDNDA#TIFMDSDLG#evqlvesggglvhagdslrlscaasgAIVHVFAmgwfrqapgkerefvasinLDNDAtnyadsvkgrftisrddarntvylqmnslkpedtavyycna
TIFMDSDLGdywgqgtqvtvss#GAAGGAGATATACCATGGAAGTTCAACTGGTTGAATCTGGTGGCGGTTTAGTTCACGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTAATGCAACCATCTTTATGGATTCCGATTTGGGTGACTATTGGGGTCAGGGCACACAAGTCACGGTCTCAAGCGGCGGAAGC
TCTGGTGAACAGAAACTGATTTCAGAAG
AIVHVFA#LDNDA#TIFMDSDLG#evqlvdsggglvhagdrIrlscaasgAIVHVFAmgwfrqapgkerefvasinLDNDAtnyadsvkgrftisrddarntvylqmnslkpedtavyycna
TIFMDSDLGdywgqgtqvtvss#GAAGGAGATATACCATGGAAGTTCAACTGGTTGACTCTGGTGGCGGTTTAGTTCACGCCGGTGATCGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTAATGCAACCATCTTTATGGATTCCGATTTGGGTGACTATTGGGGTCAGGGCACACAAGTCACGGTCTCAAGCGGCGGAAGC
TCTGGTGAACAGAAACTGATTTCAGAAG
AIVHVFA#LDNDA#TIFMDSDLG#evqlvesggglvqasdslrlscaasgAIVHVFAmgwfrqapgkerefvasinLDNDAtnyadsvkgrftisrddarntvylqmnslkpedtavyycna
TIFMDSDLGdywgqgtqvtvss#AAGGAGATATACCATGGAAGTTCAACTGGTTGAATCTGGTGGCGGTTTAGTTCAAGCCAGTGATAGCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTAATGCAACCATCTTTATGGATTCCGATTTGGGTGACTATTGGGGTCAGGGCACACAAGTCACAGTCTCAAGCGGCGGAAGCTCT
GGTGAACAGAAACTGATTTCAGAAG
AIVHVFA#LDNDA#LENPHIASKY#evqlvesggglvqagdslrlscaasgAIVHVFAmgwfrqapgkerefvasinLDNDAtnyadsvkgrftisrddahntvylqmnslkpedtavyyca
aLENPHIASKYdywgqgtqvtvss#ATACCATGGAAGTTCAACTGGTTGAATCTGGTGGCGGTTTAGTTCAAGCCGGTGATAGCCTTCGTCTGAGCTGCGCCGCA
AGCGGAGCCATTGTGCATGTGTTCGCTATGGGTTGGTTTCGCCAGGCACCTGGTAAAGAACGTGAATTTGTGGCATCCATTAACCTTGACAACGACGCTACC
AACTATGCCGATTCTGTTAAAGGTCGCTTCACTATTTCACGCGATGATGCGCATAATACCGTGTACTTACAGATGAACTCACTGAAACCTGAGGATACCGCCG
TTTATTATTGTGCGGCACTCGAGAACCCCCACATTGCGTCCAAGTATGACTATTGGGGTCAGGGCACACAAGTCACGGTCTCAAGCGGCGGAAGCTCTGGT
GAACAGAAACTGATTTCAGAAG
AIVHVFA#LDNDA#PFWINSSVV#evqlvesggglvqagdslrlrcaasgAIVHVFAmgwfrqapgkerefvasinLDNDAtnyadsvkgrftisrddahntvylqmnslkpedtavyycn
aPFWINSSVVdywgqgtqvtvss#AGGAGATATACCATGGAAGTTCAACTGGTTGAATCTGGTGGCGGTTTAGTTCAAGCCGGTGATAGCCTTCGTCTGCGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCATAATACCGTGTACTTACAGATGAACTCACTGAAACCTGAGGAT
ACCGCCGTTTATTATTGTAATGCACCCTTCTGGATCAATAGTTCTGTTGTGGACTATTGGGGTCAGGGCACACAAGTCACGGTCTCAAGCGGCGGAAGCTCT
GGTGAACAGAAACTGATTTCAGAAG
AIVHVFA#LDNDA#PFWINSSVV#evqlvesggglvqagdslrlscaasgAIVHVFAmgwfrqapgkerefvasinLDNDAtnyadsvkgrftisrddahntvflqmnslkpedtavyycn
aPFWINSSVVddwgqgtqvtvss#GAAGGAGATATACCATGGAAGTTCAACTGGTTGAATCTGGTGGCGGTTTAGTTCAAGCCGGTGATAGCCTTCGTCTGAGC
TGCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCATAATACCGTGTTCTTACAGATGAACTCACTGAAACCTGAGG
ATACCGCCGTTTATTATTGTAATGCTCCCTTCTGGATCAATAGTTCTGTTGTGGACGATTGGGGTCAGGGCACACAAGTCACGGTCTCAAGCGGCGTAAGCT
CTGGTGAATAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGeywgqgtqvtvas#ATATACCATGGAAGTTCAACTGGTTGAATCTGGTGGCGGTTTAGTTCAAGCCGGTGATAGCCTTCGTCTGAGCTGCGCCGC
AAGCGGAGCCATTGTGCATGTGTTCGCTATGGGTTGGTTTCGCCAGGCACCTGGTAAAGAACGTGAATTTGTGGCATCCATTAACCTTGACAACGACGCTAC
CAACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTAATGCAACCATCTTTATGGATTCCGATTTGGGTGAATATTGGGGGCAGGGCACACAAGTCACGGTCGCAAGCGGCGGAAGCTCTGGGGA
ACAGAAACTGATTTCAGAAG
AIVHVFA#LDNDA#TAISPISIVD#qvqlvesggglvqagdslrlscaasgAIVHVFAmgwfrqapgkerefvasinLDNDAtnyadsvkgrftisrddarntvylqmnslkpedtavyycaa
TAISPISIVDeywgqgtqvtvss#GGAGATATACCATGCAGGTTCAACTGGTTGAATCTGGTGGCGGTTTAGTTCAAGCCGGTGATAGCCTTCGTCTGAGCTGCGC
CGCAAGCGGAGCCATTGTGCATGTGTTCGCTATGGGTTGGTTTCGCCAGGCACCTGGTAAAGAACGTGAATTTGTGGCATCCATTAACCTTGACAACGACGC
TACCAACTATGCCGATTCTGTTAAAGGTCGCTTCACTATTTCACGCGATGATGCGCGTAATACCGTGTACTTACAGATGAACTCACTGAAACCTGAGGATACC
GCCGTTTATTATTGTGCGGCAACGGCTATTAGCCCTATCTCTATTGTGGATGAGTATTGGGGTCAGGGCACACAAGTCACGGTCTCAAGCGGCGGAAGCTCT
GGTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdyggqgtqvtvss#ACCATGGAAGTTCAACTGGTTGAATCTGGTGGCGGTTTAGTTCAAGCCGGTGATAGCCTTCGTCTGAGCTGCGCCGCAAGC
GGAGCCATTGTGCATGTGTTCGCTATGGGTTGGTTTCGCCAGGCACCTGGTAAAGAACGTGAATTTGTGGCATCCATTAACCTTGACAACGACGCTACCAAC
TATGCCGATTCTGTTAAAGGTCGCTTCACTATTTCACGCGATGATGCGCGTAATACCGTGTACTTACAGATGAACTCACTGAAACCTGAGGATACCGCCGTTT
ATTATTGTAATGCAACCATCTTTATGGATTCCGATTTGGGTGACTATGGGGGTCAGGGCACACAAGTCACGGTCTCAAGCGGCGGAAGCTCTGGGGAACAG
AAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdywgqgtqvtvas#ATACCATGGAAGTTCAACTGGTTGAATCTGGTGGCGGTTTAGTTCAAGCCGGTGATAGCCTTCGTCTGAGCTGCGCCGCAA
GCGGAGCCATTGTGCATGTGTTCGCTATGGGTTGGTTTCGCCAGGCACCTGGTAAAGAACGTGAATTTGTGGCATCCATTAACCTTGACAACGACGCTACCA
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTAATGCAACCATCTTTATGGATTCCGATTTGGGTGACTATTGGGGGCAGGGCACACAAGTCACGGTCGCAAGCGGCGGAAGCTCTGGGGAAC
AGAAACTGATTTCAGAAG
AIVHVFA#LDNDA#TIFMDSDLG#evqlvesggglvqagdslrlscaasgAIVHVFAmgwlrqapgkerefvasinLDNDAtnyadsvkgrftisrddarntvylqmnslkpedtavyycna
TIFMDSDLGdywgqgtqvtvss#ATACCATGGAAGTTCAACTGGTTGAATCTGGTGGCGGTTTAGTTCAAGCCGGTGATAGCCTTCGTCTGAGCTGCGCCGCAA
GCGGAGCCATTGTGCATGTGTTCGCTATGGGTTGGCTTCGCCAGGCACCTGGTAAAGAACGTGAATTTGTGGCATCCATTAACCTTGACAACGACGCTACCA
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTAATGCAACCATCTTTATGGATTCCGATTTGGGTGACTATTGGGGTCAGGGCACACAAGTCACGGTCTCAAGCGGCGGAAGCTCTGGTGAACA
GAAACTGATTTCAGAAG
AIVHVFA#LDNDA#TAISPISIVD#evqlvesggglvhagdslrlscaasgAIVHVFAmgwfrqapgkerefvasinLDNDAtnyadsvkgrftisrddarntvylqmnslkpedtavyycaa
TAISPISIVDeywgqgtqvtvss#GAAGGAGATATACCATGGAAGTTCAACTGGTTGAATCTGGTGGCGGTTTAGTTCACGCCGGTGATAGCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTGCGGCAACGGCTATTAGCCCTATCTCTATTGTGGATGAGTATTGGGGTCAGGGCACACAAGTCACGGTCTCAAGCGGCGGAAG
CTCTGGTGAACAGAAACTGATTTCAGAAG
AIVHVFA#LDNDA#WSFTPSICTL#evqlvesggglvqagdslrlscaasgAIVHVFAmgwfrqapgkerefvasinLDNDAtnyadsvkgrftisrddarntvylqmnslkpedtavyyca
aWSFTPSICTLeywgqgtqvtvsr#AGATATACCATGGAAGTTCAACTGGTTGAATCTGGTGGCGGTTTAGTTCAAGCCGGTGATAGCCTTCGTCTGAGCTGCGC
CGCAAGCGGAGCCATTGTGCATGTGTTCGCTATGGGTTGGTTTCGCCAGGCACCTGGTAAAGAACGTGAATTTGTGGCATCCATTAACCTTGACAACGACGC
TACCAACTATGCCGATTCTGTTAAAGGTCGCTTCACTATTTCACGCGATGATGCGCGTAATACCGTGTACTTACAGATGAACTCACTGAAACCTGAGGATACC
GCCGTTTATTATTGTGCGGCATGGAGTTTCACCCCTTCTATCTGTACCTTGGAGTATTGGGGTCAGGGCACACAAGTCACGGTCTCAAGGGGCGGAAGCTCT
GGTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftnsrddarntvylqmnslkpedtavyycn
aTIFMDSDLGdywgqgtqvtvss#AAGGAGATATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTAATTCACGCGATGATGCGCGTAATACCGTGTACTTACAGATGAACTCACTGAAACCTGAG
GATACCGCCGTTTATTATTGTAATGCAACCATCTTTATGGATTCCGATTTGGGTGACTATTGGGGTCAGGGCACACAAGTCACGGTCTCAAGCGGCGGAAGC
TCTGGTGAACAGAAACTGATTTCAGAAG
FCYVPLATCaywgqgtqvtvss#ATACCATGGAAGTTCAACTGGTTGAATCTGGTGGCGGTTTAGTTCAAGCCGGTGATAGCCTTCGTCTGAGCTGCGCCGCAAG
AIVHVFA#LDNDA#FCYVPLATC#evqlvesggglvqagdslrlscaasgAIVHVFAmgwfrqapgkerefvasinLDNDAtnyadsvkgrftisrddarntvylqmnslkpedtavyycna
CGGAGCCATTGTGCATGTGTTCGCTATGGGTTGGTTTCGCCAGGCACCTGGTAAAGAACGTGAATTTGTGGCATCCATTAACCTTGACAACGACGCTACCAA
CTATGCCGATTCTGTTAAAGGTCGCTTCACTATTTCACGCGATGATGCGCGTAATACCGTGTACTTACAGATGAACTCACTGAAACCTGAGGATACCGCCGTT
TATTATTGTAATGCATTCTGCTACGTCCCTTTGGCCACGTGTGCCTATTGGGGTCAGGGCACACAAGTCACGGTCTCAAGCGGCGGAAGCTCTGGTGAACAG
AAACTGATTTCAGAAG
AIVHVFA#LDNDA#ACSDYTYGSHGVR#evqlvesggglvqagdslrlscaasgAIVHVFAmgwfrqapgkerkfvasinLDNDAtnyadsvkgrftisrddarntvylqmnslkpedtav
yycnaACSDYTYGSHGVRdswgqgtqvtvss#ACCATGGAAGTTCAACTGGTTGAATCTGGTGGCGGTTTAGTTCAAGCCGGTGATAGCCTTCGTCTGAGCTGCG
CCGCAAGCGGAGCCATTGTGCATGTGTTCGCTATGGGTTGGTTTCGCCAGGCACCTGGTAAAGAACGTAAATTTGTGGCATCCATTAACCTTGACAACGACG
CTACCAACTATGCCGATTCTGTTAAAGGTCGCTTCACTATTTCACGCGATGATGCGCGTAATACCGTGTACTTACAGATGAACTCACTGAAACCTGAGGATAC
CGCCGTTTATTATTGTAATGCAGCGTGTTCGGACTATACGTATGGCTCTCATGGTGTTCGGGACTCTTGGGGTCAGGGCACACAAGTCACGGTCTCAAGCGG
CGGAAGCTCTGGTGAACAGAAACTGATTTCAGAAG
AIAHVFA#LDNDA#TIFMDSDLG#evqlvesggglvqagdslrlscaasgAIAHVFAmgwfrqapgkerefvasinLDNDAtnyadsvkgrftisrddarntvylqmnslkpedtavyycn
aTIFMDSDLGdywgqgtqvtvss#GGAGATATACCATGGAAGTTCAACTGGTTGAATCTGGTGGCGGTTTAGTTCAAGCCGGTGATAGCCTTCGTCTGAGCTGC
GCCGCAAGCGGAGCCATTGCGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTAATGCAACCATCTTTATGGATTCCGATTTGGGTGACTATTGGGGTCAGGGCACACAAGTCACGGTCTCAAGCGGCGGAAGCTCT
GGTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdywghgtqvtvss#ACCATGGAAGTTCAACTGGTTGAATCTGGTGGCGGTTTAGTTCAAGCCGGTGATAGCCTTCGTCTGAGCTGCGCCGCAAGC
GGAGCCATTGTGCATGTGTTCGCTATGGGTTGGTTTCGCCAGGCACCTGGTAAAGAACGTGAATTTGTGGCATCCATTAACCTTGACAACGACGCTACCAAC
TATGCCGATTCTGTTAAAGGTCGCTTCACTATTTCACGCGATGATGCGCGTAATACCGTGTACTTACAGATGAACTCACTGAAACCTGAGGATACCGCCGTTT
ATTATTGTAATGCAACCATCTTTATGGATTCCGATTTGGGTGACTATTGGGGTCATGGCACACAAGTCACGGTCTCAAGCGGCGGAAGCTCTGGTGAACAGA
AACTGATTTCAGAAG
AIVHVFA#LDNDA#TIFMDSDLG#evqlvesggglvpagdslrlscaasgAIVHVFAmgwfrqapgkerefvasinLDNDAtnyadsvkgrftisrddarntvylqmnslkpedtavyycna
TIFMDSDLGdywgqgtqvtvss#GGAGATATACCATGGAAGTTCAACTGGTTGAATCTGGTGGCGGTTTAGTTCCAGCCGGTGATAGCCTTCGTCTGAGCTGCG
CCGCAAGCGGAGCCATTGTGCATGTGTTCGCTATGGGTTGGTTTCGCCAGGCACCTGGTAAAGAACGTGAATTTGTGGCATCCATTAACCTTGACAACGACG
CTACCAACTATGCCGATTCTGTTAAAGGTCGCTTCACTATTTCACGCGATGATGCGCGTAATACCGTGTACTTACAGATGAACTCACTGAAACCTGAGGATAC
CGCCGTTTATTATTGTAATGCAACCATCTTTATGGATTCCGATTTGGGTGACTATTGGGGTCAGGGCACACAAGTCACGGTCTCAAGCGGCGGAAGCTCTGG
TGAACAGAAACTGATTTCAGAAG
AIVHVFA#LDNDA#PFWINSSVV#evqlvesggglvqagdslrlscaasgAIVHVFAmgwfrqapgkerefvasinLDNDAtnyadsvkgrftisrddarntvylqmnslkpedtavyycn
aPFWINSSVVdywgqgtqvtvss#AGGAGATATACCATGGAAGTTCAACTGGTTGAATCTGGTGGCGGTTTAGTTCAAGCCGGTGATAGCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTAATGCACCCTTCTGGATCAATAGTTCTGTTGTGGACTATTGGGGTCAGGGCACACAAGTCACGGTCTCAAGCGGCGGAAGCTCT
GGTGAACAGAAACTGATTTCAGAAG
AIVHVFA#LDNDA#PFWINSSVV#evqlvesggglvqagdslrlscaasgAIVHVFAmgwfrqapgkerefvssinLDNDAtnyadsvkgrftisrddahntvylqmnslkledtpvyycna
PFWINSSVVdywgqgtqvtvsr#GAAGGAGAGATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGTCATCCATTAACCTTGACAACG
ACGCTACCAACTATGCCGATTCTGTTAAAGGTCGCTTCACTATTTCACGCGATGATGCGCATAATACCGTGTACTTACAGATGAACTCACTGAAACTTGAGGA
TACCCCCGTTTATTATTGCAATGCCCCCTTCTGGATCAATAGTTCTGTTGTGGACTATTGGGGTCAGGGCACACAAGTCACGGTCTCACGCGGCGGAAGCTCT
GGTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdywfqgtqvtvsr#AGGAGATATACCATGGAAGTTCAACTGGTTGAATCTGGTGGCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTAATGCAACCATCTTTATGGATTCCGATTTGGGTGACTATTGGTTTCAGGGCACACAAGTCACGGTCTCACGCGGCGGAAGCTCTGG
TGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedttvyycna
TIFMDSDLGdywgqgtqvtvss#ATATACCATGGAAGTTCAACTGGTTGAATCTGGTGGCGGTTTAGTTCAAGCCGGTGATAGCCTTCGTCTGAGCTGCGCCGC
AAGCGGAGCCATTGTGCATGTGTTCGCTATGGGTTGGTTTCGCCAGGCACCTGGTAAAGAACGTGAATTTGTGGCATCCATTAACCTTGACAACGACGCTAC
CAACTATGCCGATTCTGTTAAAGGTCGCTTCACTATTTCACGCGATGATGCGCGTAATACCGTGTACTTACAGATGAACTCACTGAAACCTGAGGATACCACC
GTTTATTATTGTAATGCAACCATCTTTATGGATTCCGATTTGGGTGACTATTGGGGTCAGGGCACACAAGTCACGGTCTCAAGCGGCGGAAGCTCTGGTGAA
CAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdywgqgthvtvss#AGATATACCATGGAAGTTCAACTGGTTGAATCTGGTGGCGGTTTAGTTCAAGCCGGTGATAGCCTTCGTCTGAGCTGCGCC
GCAAGCGGAGCCATTGTGCATGTGTTCGCTATGGGTTGGTTTCGCCAGGCACCTGGTAAAGAACGTGAATTTGTGGCATCCATTAACCTTGACAACGACGCT
ACCAACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTAATGCAACCATCTTTATGGATTCCGATTTGGGTGACTATTGGGGTCAGGGCACACATGTCACGGTCTCAAGCGGCGGAAGCTCTGGTG
AACAGAAACTGATTTCAGAAG
AIVHVFA#LDNDA#PFWINSSVV#evqlvesggglvqagdslrlscaasgAIVHVFAmgwfrqapgkerefvapinLDNDAtnyadsvkgrftisrddahntvylqmnslkpedtavyycn
aPFWINSSVVdywgqgtqvtvss#GGAGATATACCATGGAAGTTCAACTGGTTGAATCTGGTGGCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCACCCATTAACCTTGACAACGA
CGCTACCAACTATGCCGATTCTGTTAAAGGTCGCTTCACTATTTCACGCGATGATGCGCATAATACCGTGTACTTACAGATGAACTCACTGAAACCTGAGGAT
ACCGCCGTTTATTATTGTAATGCACCCTTCTGGATCAATAGTTCTGTTGTGGACTATTGGGGTCAGGGCACACAAGTCACGGTCTCAAGCGGCGGAAGCTCT
TGTGAACAGACACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdywgqgtqvtvsr#AAGGAGATATACCATGGAAGTTCAACTGGTTGAATCTGGTGGCGGTTTAGTTCAAGCCGGTGATAGCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTAATGCAACCATCTTTATGGATTCCGATTTGGGTGACTATTGGGGTCAGGGCACACAAGTCACGGTCTCACGCGGCGGAAGCTCT
GGTGAACAGAAACTGATTTCAGAAG
AIVHVFA#LDNDA#TIFMDSDLG#evqlvesggglvqagdslrlscaasgAIVHVFAmgwfrrapgkerefvasinLDNDAtnyadsvkgrftisrddarntvylqmnslkpedtavyycna
TIFMDSDLGdywgqgtqvtvss#AGATATACCATGGAAGTTCAACTGGTTGAATCTGGTGGCGGTTTAGTTCAAGCCGGTGATAGCCTTCGTCTGAGCTGCGCC
GCAAGCGGAGCCATTGTGCATGTGTTCGCTATGGGTTGGTTTCGCCGGGCACCTGGTAAAGAACGTGAATTTGTGGCATCCATTAACCTTGACAACGACGCT
ACCAACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTAATGCAACCATCTTTATGGATTCCGATTTGGGTGACTATTGGGGTCAGGGCACACAAGTCACGGTCTCAAGCGGCGGAAGCTCTGGTG
AACAGAAACTGATTTCAGAAG
AIVHVFA#LDNDA#ISLSLDSCLQ#evqmvesggglvqagdslrlscaasgAIVHVFAmgwfrqapgkerefvasinLDNDAtnyadsvkgrftisrddarntvylqmnslkpedtavyyca
rISLSLDSCLQeywgqgtqvtvss#AGATATACCATGGAAGTTCAAATGGTTGAATCTGGTGGCGGTTTAGTTCAAGCCGGTGATAGCCTTCGTCTGAGCTGCGCC
GCAAGCGGAGCCATTGTGCATGTGTTCGCTATGGGTTGGTTTCGCCAGGCACCTGGTAAAGAACGTGAATTTGTGGCATCCATTAACCTTGACAACGACGCT
ACCAACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTGCGCGTATTTCTTTGAGCCTGGATTCCTGTCTGCAGGAGTATTGGGGTCAGGGCACACAAGTCACGGTCTCAAGCGGCGGAAGCTCTG
GTGAACAGAAACTGATTTCAGAAG
AIVHVFA#LDNDA#PFWINSSVV#evqlvesggglvqagdslrlscaasgAIVHVFAmgwfrqapgkerefvasinLDNDAtnyadsvkgrftisrddahntmylqmnslkpedtavyyc
naPFWINSSVVdywgqgtqvtvss#AGATATACCATGGAAGTTCAACTGGTTGAATCTGGTGGCGGTTTAGTTCAAGCCGGTGATAGCCTTCGTCTGAGCTGCGC
CGCAAGCGGAGCCATTGTGCATGTGTTCGCTATGGGTTGGTTTCGCCAGGCACCTGGTAAAGAACGTGAATTTGTGGCATCCATTAACCTTGACAACGACGC
TACCAACTATGCCGATTCTGTTAAAGGTCGCTTCACTATTTCACGCGATGATGCGCATAATACCATGTACTTACAGATGAACTCACTGAAACCTGAGGATACC
GCCGTTTATTATTGTAATGCACCCTTCTGGATCAATAGTTCTGTTGTGGACTATTGGGGTCAGGGCACACAAGTCACGGTCTCAAGCGGCGGAAGCTCTGGT
GAACAGAAACTGATTTCAGAAG
AIVHVFA#LDNDA#PFWINSSVV#evqlvesggglvqagdslrlscaasgAIVHVFAmgwlrqapgkerefvasinLDNDAtnyadsvkgrftisrddahntvylqmnslkpedtavyycn
aPFWINSSVVdycgqgtqvtvss#ACCATGGAAGTTCAACTGGTTGAATCTGGTGGCGGTTTAGTTCAAGCCGGTGATAGCCTTCGTCTGAGCTGCGCCGCAAGC
GGAGCCATTGTGCATGTGTTCGCTATGGGTTGGCTTCGCCAGGCACCTGGTAAAGAACGTGAATTTGTGGCATCCATTAACCTTGACAACGACGCTACCAAC
TATGCCGATTCTGTTAAAGGTCGCTTCACTATTTCACGCGATGATGCGCATAATACCGTGTACTTACAGATGAACTCACTGAAACCTGAGGATACCGCCGTTT
ATTATTGTAATGCACCCTTCTGGATCAATAGTTCTGTTGTGGACTATTGTGGTCAGGGCACACAAGTCACGGTCTCAAGCGGCGGAAGCTCTGGTGAACAGA
AACTGATTTCAGAAG
AIVHVFA#LDNDA#TIFMDSDLG#evqlvesggglvqagdslrlscaasgAIVHVFAmgwfrqapgkehefvasinLDNDAtnyadsvkgrftisrddarntvylqmnslkpedtavyycn
aTIFMDSDLGdywgqgtqvtvss#AGATATACCATGGAAGTTCAACTGGTTGAATCTGGTGGCGGTTTAGTTCAAGCCGGTGATAGCCTTCGTCTGAGCTGCGCC
GCAAGCGGAGCCATTGTGCATGTGTTCGCTATGGGTTGGTTTCGCCAGGCACCTGGTAAAGAACATGAATTTGTGGCATCCATTAACCTTGACAACGACGCT
ACCAACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTAATGCAACCATCTTTATGGATTCCGATTTGGGTGACTATTGGGGTCAGGGCACACAAGTCACGGTCTCAAGCGGCGGAAGCTCTGGTG
AACAGAAACTGATTTCAGAAG
DIVHVFA#LDNDA#TIFMDSDLG#evqlvesggglvqagdslrlscaasgDIVHVFAmgwfrqapgkerefvasinLDNDAtnyadsvkgrftisrddarntvylqmnslkpedtavyycn
aTIFMDSDLGdywgqgtqvtvss#ACCATGGAAGTTCAACTGGTTGAATCTGGTGGCGGTTTAGTTCAAGCCGGTGATAGCCTTCGTCTGAGCTGCGCCGCAAG
CGGAGACATTGTGCATGTGTTCGCTATGGGTTGGTTTCGCCAGGCACCTGGTAAAGAACGTGAATTTGTGGCATCCATTAACCTTGACAACGACGCTACCAA
CTATGCCGATTCTGTTAAAGGTCGCTTCACTATTTCACGCGATGATGCGCGTAATACCGTGTACTTACAGATGAACTCACTGAAACCTGAGGATACCGCCGTT
TATTATTGTAATGCAACCATCTTTATGGATTCCGATTTGGGTGACTATTGGGGTCAGGGCACACAAGTCACGGTCTCAAGCGGCGGAAGCTCTGGTGAACAG
AAACTGATTTCAGAAG
AIVHVFA#LDNDA#TIFMDSDLG#evqlvesggglvqdgdslrlscaasgAIVHVFAmgwfrqapgkerefvasinLDNDAtnyadsvkgrftisrddarntvylqmnslkpedtavyycn
aTIFMDSDLGdywgqgtqvtvss#ATATACCATGGAAGTTCAACTGGTTGAATCTGGTGGCGGTTTAGTTCAAGACGGTGATAGCCTTCGTCTGAGCTGCGCCGC
AAGCGGAGCCATTGTGCATGTGTTCGCTATGGGTTGGTTTCGCCAGGCACCTGGTAAAGAACGTGAATTTGTGGCATCCATTAACCTTGACAACGACGCTAC
CAACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTAATGCAACCATCTTTATGGATTCCGATTTGGGTGACTATTGGGGTCAGGGCACACAAGTCACGGTCTCAAGCGGCGGAAGCTCTGGTGAA
CAGAAACTGATTTCAGAAG
AIVHVFA#LDTDA#TIFMDSDLG#evqlvesggglvqagdslrlscaasgAIVHVFAmgwfrqapgkerefvasinLDTDAtnyadsvkgrftisrddarntvylqmnslkpedtavyycna
TIFMDSDLGdywgqgtqvtvss#ATATACCATGGAAGTTCAACTGGTTGAATCTGGTGGCGGTTTAGTTCAAGCCGGTGATAGCCTTCGTCTGAGCTGCGCCGC
AAGCGGAGCCATTGTGCATGTGTTCGCTATGGGTTGGTTTCGCCAGGCACCTGGTAAAGAACGTGAATTTGTGGCATCCATTAACCTTGACACCGACGCTAC
CAACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTAATGCAACCATCTTTATGGATTCCGATTTGGGTGACTATTGGGGTCAGGGCACACAAGTCACGGTCTCAAGCGGCGGAAGCTCTGGTGAA
CAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnplkpedtavyycn
aTIFMDSDLGdywgqgtqvtvss#ACCATGGAAGTTCAACTGGTTGAATCTGGTGGCGGTTTAGTTCAAGCCGGTGATAGCCTTCGTCTGAGCTGCGCCGCAAG
CGGAGCCATTGTGCATGTGTTCGCTATGGGTTGGTTTCGCCAGGCACCTGGTAAAGAACGTGAATTTGTGGCATCCATTAACCTTGACAACGACGCTACCAA
CTATGCCGATTCTGTTAAAGGTCGCTTCACTATTTCACGCGATGATGCGCGTAATACCGTGTACTTACAGATGAACCCACTGAAACCTGAGGATACCGCCGTT
TATTATTGTAATGCAACCATCTTTATGGATTCCGATTTGGGTGACTATTGGGGTCAGGGCACACAAGTCACGGTCTCAAGCGGCGGAAGCTCTGGTGAACAG
AAACTGATTTCAGAAG
AIVHVFA#LDNDA#TIFMDSDLG#evqlvesggglvqagdslslscaasgAIVHVFAmgwfrqapgkerefvasinLDNDAtnyadsvkgrftisrddarntvylqmnslkpedtavyycn
aTIFMDSDLGdywgqgtqvtvss#AAGGAGATATACCATGGAAGTTCAACTGGTTGAATCTGGTGGCGGTTTAGTTCAAGCCGGTGATAGCCTTAGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTAATGCAACCATCTTTATGGATTCCGATTTGGGTGACTATTGGGGTCAGGGCACACAAGTCACGGTCTCAAGCGGCGGAAGC
TCTGGTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddahntvylqmnslkpedtavyycn
aTIFMDSDLGdywgqgtqvtvss#ACCATGGAAGTTCAACTGGTTGAATCTGGTGGCGGTTTAGTTCAAGCCGGTGATAGCCTTCGTCTGAGCTGCGCCGCAAG
CGGAGCCATTGTGCATGTGTTCGCTATGGGTTGGTTTCGCCAGGCACCTGGTAAAGAACGTGAATTTGTGGCATCCATTAACCTTGACAACGACGCTACCAA
CTATGCCGATTCTGTTAAAGGTCGCTTCACTATTTCACGCGATGATGCGCATAATACCGTGTACTTACAGATGAACTCACTGAAACCTGAGGATACCGCCGTT
TATTATTGTAATGCAACCATCTTTATGGATTCCGATTTGGGTGACTATTGGGGTCAGGGCACACAAGTCACGGTCTCAAGCGGCGGAAGCTCTGGTGAACAG
AAACTGATTTCAGAAG
AIVHVLA#LDNDA#TIFMDSDLG#evqlvesggglvqagdslrlscaasgAIVHVLAmgwfrqapgkerefvasinLDNDAtnyadsvkgrftisrddarntvylqmnslkpedtavyycna
TIFMDSDLGdywgqgtqvtvss#ATACCATGGAAGTTCAACTGGTTGAATCTGGTGGCGGTTTAGTTCAAGCCGGTGATAGCCTTCGTCTGAGCTGCGCCGCAA
GCGGAGCCATTGTGCATGTGTTAGCTATGGGTTGGTTTCGCCAGGCACCTGGTAAAGAACGTGAATTTGTGGCATCCATTAACCTTGACAACGACGCTACCA
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTAATGCAACCATCTTTATGGATTCCGATTTGGGTGACTATTGGGGTCAGGGCACACAAGTCACGGTCTCAAGCGGCGGAAGCTCTGGTGAACA
GGAACTGATTTCAGAAG
AIVHVFA#LDNDA#ESTSYF#evqlvesggglvqagdslrlscaasgAIVHVFAmgwfrqapgkerefvasinLDNDAtnyadsvkgrftisrddarntvylqmnslkpedtavyycavESTS
YFgywgqgtqvtvss#AAGGAGATATACCATGGAAGTTCAACTGGTTGAATCTGGTGGCGGTTTAGTTCAAGCCGGTGATAGCCTTCGTCTGAGCTGCGCCGCA
AGCGGAGCCATTGTGCATGTGTTCGCTATGGGTTGGTTTCGCCAGGCACCTGGTAAAGAACGTGAATTTGTGGCATCCATTAACCTTGACAACGACGCTACC
AACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGTTGAGAGTACGAGTTATTTTGGCTATTGGGGTCAGGGCACACAAGTCACGGTCTCAAGCGGCGGAAGCTCTGGTGAACAGAAACT
GATTTCAGAAG
AIVHVFA#LDNDA#PFWINSSVV#qvqlvesggglvqagdslrlscassgAIVHVFAmgwfrqapgkerefvasinLDNDAtnyadsvkgrftisrddahntvylqmnslkpedtavyycn
aPFWINSSVVdywgqgtqvtvss#AGGAGATATACCATGCAGGTTCAACTGGTTGAATCTGGTGGCGGTTTAGTTCAAGCCGGTGATAGCCTTCGTCTGAGCTG
CGCCTCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCATAATACCGTGTACTTACAGATGAACTCACTGAAACCTGAGGAT
ACCGCCGTTTATTATTGTAATGCACCCTTCTGGATCAATAGTTCTGTTGTGGACTATTGGGGTCAGGGCACACAAGTCACGGTCTCAAGCGGCGGAAGCTCT
GGTGAACAGAAACTGATTTCAGAAG
AIVHVFA#LDNDA#FCYVPLATC#evqlvesggglvqagdslrlscaasgAIVHVFAmgwfrqapgkerefvasinLDNDAtnyadsvkgrftisrddarntvylqmnslkpedtavyycna
FCYVPLATCdywgqdtqvtvss#GGAGATATACCATGGAAGTTCAACTGGTTGAATCTGGTGGCGGTTTAGTTCAAGCCGGTGATAGCCTTCGTCTGAGCTGCG
CCGCAAGCGGAGCCATTGTGCATGTGTTCGCTATGGGTTGGTTTCGCCAGGCACCTGGTAAAGAACGTGAATTTGTGGCATCCATTAACCTTGACAACGACG
CTACCAACTATGCCGATTCTGTTAAAGGTCGCTTCACTATTTCACGCGATGATGCGCGTAATACCGTGTACTTACAGATGAACTCACTGAAACCTGAGGATAC
CGCCGTTTATTATTGTAATGCATTCTGCTACGTCCCTTTGGCCACGTGTGACTATTGGGGTCAGGACACACAAGTCACGGTCTCAAGCGGCGGAAGCTCTGG
TGAACAGAAACTGATTTCAGAAG
AIVHVFA#LDNDA#PFWINSSVV#evqlvesggglvqagdslrlscaasgAIVHVFAmgwfrqapgkerefvasinLDNDAtnyadsvkgrftisrddahntvylqmnslkpqdtavyycn
aPFWINSSVVdywgqgtqvtvss#AGGAGATATACCATGGAAGTTCAACTGGTTGAATCTGGTGGCGGTTTAGTTCAAGCCGGTGATAGCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCATAATACCGTGTACTTACAGATGAACTCACTGAAACCTCAGGAT
ACCGCCGTTTATTATTGTAATGCACCCTTCTGGATCAATAGTTCTGTTGTGGACTATTGGGGTCAGGGCACACAAGTCACGGTCTCAAGCGGCGGAAGCTCT
GGTGAACAGAAACTGCTTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddacntvylqmnslkpedtavyycn
aTIFMDSDLGdywgqgtqvtvss#AGGAGATATACCATGGAAGTTCAACTGGTTGAATCTGGTGGCGGTTTAGTTCAAGCCGGTGATAGCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGTGTAATACCGTGTACTTACAGATGAACTCACTGAAACCTGAGGAT
ACCGCCGTTTATTATTGTAATGCAACCATCTTTATGGATTCCGATTTGGGTGACTATTGGGGTCAGGGCACACAAGTCACGGTCTCAAGCGGCGGAAGCTCT
GGTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyvdsvkgrftisrddarntvylqmnslkpedtavyycna
TIFMDSDLGdywvqgtqvtvss#AGGAGATATACCATGGAAGTTCAACTGGTTGAATCTGGTGGCGGTTTAGTTCAAGCCGGTGATAGCCTTCGTCTGAGCTGC
GCCGCAAGCGGAGCCATTGTGCATGTGTTCGCTATGGGCTGGTTTCGCCAGGCACCTGGTAAAGAACGTGAATTTGTGGCATCCATTAACCTTGACAACGA
CGCTACCAACTATGTCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTAATGCAACCATCTTTATGGATTCCGATTTGGGTGACTATTGGGTTCAGGGCACACAAGTCACGGTCTCAAGCGGCGGAAGCTCT
GTTGAACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdywgqstqvtvss#AGGAGATATACCATGGAAGTTCAACTGGTTGAATCTGGTGGCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAGGGTCGCTTCACTATTTCACGCGATGATGCGCGTAATACCGTGTACTTACAGATGAACTCACTGAAACCTGAGGATA
CCGCCGTTTATTATTGTAATGCAACCATCTTTATGGATTCCGATTTGGGTGACTATTGGGGTCAGAGCACACAAGTCACGGTCTCAAGCGGCGGAAGCTCTG
GTGAACAGAAACTGATTTCAGAAG
AIVHVFA#LDNDA#PFWINSSVV#evqlvesggglvqagdslrlscaasgAIVHVFAmgwfrqapgkerefvasvnLDNDAtnyadsvkgrftisrddahntvylqmnslkpedtavyycn
aPFWINSSVVdywgqgtqvtvss#AAGGAGATATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCGTTAACCTTGACAAC
GACGCTACCAACTATGCCGATTCTGTTAAAGGTCGCTTCACTATTTCACGCGATGATGCGCATAATACCGTGTACTTACAGATGAACTCACTGAAACCTGAGG
ATACCGCCGTTTATTATTGTAATGCACCCTTCTGGATCAATAGTTCTGTTGTGGACTATTGGGGTCAGGGCACACAAGTCACGGTCTCAAGCGGCGGAAGCT
CTGGTGAACAGAAACTGATTTCAGAAG
AIVHVFA#LDNDA#TIFMDSDLG#evqlfesggglvqagdslrlscaasgAIVHVFAmgwfrqapgkerefvasinLDNDAtnyadsvkgrftisrddarntvylqmnslkpedtavyycna
TIFMDSDLGdywvqgtqvtvss#ATATACCATGGAAGTTCAACTGTTTGAATCTGGTGGCGGTTTAGTTCAAGCCGGTGATAGCCTTCGTCTGAGCTGCGCCGCA
AGCGGAGCCATTGTGCATGTGTTCGCTATGGGTTGGTTTCGCCAGGCACCTGGTAAAGAACGTGAATTTGTGGCATCCATTAACCTTGACAACGACGCTACC
AACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTAATGCAACCATCTTTATGGATTCCGATTTGGGTGACTATTGGGTTCAGGGCACACAAGTCACGGTCTCAAGCGGCGGAAGCTCTGGTGAA
CAGAAACTGATTTCAGAAG
AIVHVFA#LDNDA#TIFMDSDLG#evqlvesggglvqagdslrlscaasgAIVHVFAmgwfrqepgkerefvasinLDNDAtnyadsvkgrftisrddarntvylqmnslkpedtavyycna
TIFMDSDLGdywgqgtqvtvss#AGATATACCATGGAAGTTCAACTGGTTGAATCTGGTGGCGGTTTAGTTCAAGCCGGTGATAGCCTTCGTCTGAGCTGCGCC
GCAAGCGGAGCCATTGTGCATGTGTTCGCTATGGGTTGGTTTCGCCAGGAACCTGGTAAAGAACGTGAGTTTGTGGCATCCATTAACCTTGACAACGACGC
TACCAACTATGCCGATTCTGTTAAAGGTCGCTTCACTATTTCACGCGATGATGCGCGTAATACCGTGTACTTACAGATGAACTCACTGAAACCTGAGGATACC
GCCGTTTATTATTGTAATGCAACCATCTTTATGGATTCCGATTTGGGTGACTATTGGGGTCAGGGCACACAAGTCACGGTCTCAAGCGGCGGAAGCTCTGGT
GAACAGAAACTGATTTCAGAAG
AIVHVFA#LDNDA#PFWINSSVV#evqlvesggglvqagdslrlscaasgAIVHVFAmgwfrqapgkerefvtsinLDNDAtnyadsvkgrftisrddahntvylqmnslkpedtavyycn
aPFWINSSVVdywgqgtqvtvss#ATACCATGGAAGTTCAACTGGTTGAATCTGGTGGCGGTTTAGTTCAAGCCGGTGATAGCCTTCGTCTGAGCTGCGCCGCA
AGCGGAGCCATTGTGCATGTGTTCGCTATGGGTTGGTTTCGCCAGGCACCTGGTAAAGAACGTGAATTTGTGACATCCATTAACCTTGACAACGACGCTACC
AACTATGCCGATTCTGTTAAAGGTCGCTTCACTATTTCACGCGATGATGCGCATAATACCGTGTACTTACAGATGAACTCACTGAAACCTGAGGATACCGCCG
TTTATTATTGTAATGCACCCTTCTGGATCAATAGTTCTGTTGTGGACTATTGGGGTCAGGGCACACAAGTCACGGTCTCAAGCGGCGGAAGCTCTGGTGAAC
AGAAACTGATTTCAGAAG
AIVHVFA#LDNDA#TIFMDSDLG#evqlvesggglvqagdtIrlscaasgAIVHVFAmgwfrqapgkerefvasinLDNDAtnyadsvkgrftisrddarntvylqmnslkpedtavyycna
TIFMDSDLGdywgqgtqvtvss#AAGGAGATATACCATGGAAGTTCAACTGGTTGAATCTGGTGGCGGTTTAGTTCAAGCCGGTGATACCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTAATGCAACCATCTTTATGGATTCCGATTTGGGTGACTATTGGGGTCAGGGCACACAAGTCACGGTCTCAAGCGGCGGAAGCTCT
GGTGAACAGAAACTGATTTCAGAAG
AIVHVFA#LDNDA#TAISPISIVD#evqlvesggglvqagdslrlswaasgAIVHVFAmgwfrqapgkerefvasinLDNDAtnyadsvkgrftisrddarntvylqmnslkpedtavyycaa
TAISPISIVDeywgqgtqvtvss#ATATACCATGGAAGTTCAACTGGTTGAATCTGGTGGCGGTTTAGTTCAAGCCGGTGATAGCCTTCGTCTGAGCTGGGCCGCA
AGCGGAGCCATTGTGCATGTGTTCGCTATGGGTTGGTTTCGCCAGGCACCTGGTAAAGAACGTGAATTTGTGGCATCCATTAACCTTGACAACGACGCTACC
AACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGCAACGGCTATTAGCCCTATCTCTATTGTGGATGAGTATTGGGGTCAGGGCACACAAGTCACGGTCTCAAGCGGCGGAAGCTCTGGT
GAACAGAAACTGATTTCAGAAG
AIVHVFA#LDNDA#TAISPISIVD#evqlvesggglvqagdslrlscaasgAIVHVFAmgwfcqapgkerefvasinLDNDAtnyadsvkgrftisrddarntvylqmnslkpedtavyycaa
TAISPISIVDeywgqgtqvtvss#ATATACCATGGAAGTTCAACTGGTTGAATCTGGTGGCGGTTTAGTTCAAGCCGGTGATAGCCTTCGTCTGAGCTGCGCCGCA
AGCGGAGCCATTGTGCATGTGTTCGCTATGGGTTGGTTTTGCCAGGCACCTGGTAAAGAACGTGAATTTGTGGCATCCATTAACCTTGACAACGACGCTACC
AACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTGCGGCAACGGCTATTAGCCCTATCTCTATTGTGGATGAGTATTGGGGTCAGGGCACACAAGTCACGGTCTCAAGCGGCGGAAGCTCTGGT
GAACAGAAACTGATTTCAGAAG
AIVHVFA#LDNDA#TAISPISIVD#evqlvesggglvqagdslrlscaasgAIVHVFAmgwfrqapgkerefvasinLDNDAtnyadsvkgrftisrddarntvylqmnslkpedtavyycaa
TAISPISIVDeywvqgtqvtvss#ACCATGGAAGTTCAACTGGTTGAATCTGGTGGCGGTTTAGTTCAAGCCGGTGATAGCCTTCGTCTGAGCTGCGCCGCAAGCG
GAGCCATTGTGCATGTGTTCGCTATGGGTTGGTTTCGCCAGGCACCTGGTAAAGAACGTGAATTTGTGGCATCCATTAACCTTGACAACGACGCTACCAACT
ATGCCGATTCTGTTAAAGGTCGCTTCACTATTTCACGCGATGATGCGCGTAATACCGTGTACTTACAGATGAACTCACTGAAACCTGAGGATACCGCCGTTTA
TTATTGTGCGGCAACGGCTATTAGCCCTATCTCTATTGTGGATGAGTATTGGGTTCAGGGCACACAAGTCACGGTCTCAAGCGGCGGAAGCTCTGTTGAACA
GAAACTGATTTCAGAAG
AIVHVFA#LDNDA#TIFMDSNLG#evqlvesggglvqagdslrlscaasgAIVHVFAmgwfrqapgkerefvasinLDNDAtnyadsvkgrftisrddarntvylqmnslkpedtavyycn
aTIFMDSNLGdywgqgtqvtvss#ATATACCATGGAAGTTCAACTGGTTGAATCTGGTGGCGGTTTAGTTCAAGCCGGTGATAGCCTTCGCCTGAGCTGCGCCG
CAAGCGGAGCCATTGTGCATGTGTTCGCTATGGGTTGGTTTCGCCAGGCACCTGGTAAAGAACGTGAATTTGTGGCATCCATTAACCTTGACAACGACGCTA
CCAACTATGCCGATTCTGTTAAAGGTCGCTTCACTATTTCACGCGATGATGCGCGTAATACCGTGTACTTACAGATGAACTCACTGAAACCTGAGGATACCGC
CGTTTATTATTGTAATGCAACCATCTTTATGGATTCCAATTTGGGTGACTATTGGGGTCAGGGCACACAAGTCACGGTCTCAAGCGGCGGAAGCTCTGGTGA
ACAGAAACTGATTTCAGAAG
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdywsqgtqvtvss#AGGAGATATACCATGGAAGTTCAACTGGTTGAATCTGGTGGCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTACTATTGTAATGCAACCATCTTTATGGATTCCGATTTGGGTGACTATTGGAGTCAGGGCACACAAGTCACGGTCTCAAGCGGCGGAAGCTCTG
GTGAACAGAAACTGATTTCAGAAG
AIVHVFA#LDNDA#TLYHTIREAR#evqlvesggglvqagdslrlscaasgAIVHVFAmgwfrqapgkerefvasinLDNDAtnyadsvkgrstisrddarntvylqmnslkpedtavyycat
TLYHTIREARdywgqgtqvtvss#GAAGGAGATATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTCCACTATTTCACGCGATGATGCGCGTAATACCGTGTACTTACAGATGAACTCACTGAAACCTGAG
GATACCGCCGTTTATTATTGTGCGACAACTCTGTACCATACGATTCGGGAGGCCCGCGACTATTGGGGTCAGGGCACACAAGTCACGGTCTCAAGCGGCGG
AAGCTCTGGTGAACAGAAACTGATTTCAGAAG
AIVHVFA#LDNDA#TAISPISIVD#evqlvesggglvqagdslrlscaasgAIVHVFAmgwfrqapgkerefvasinLDNDAtnyansvkgrftisrddarntvylqmnslkpedtavyycaa
TAISPISIVDeywgqgtqvtvss#GAAGGAGATATACCATGGAAGTTCAACTGGTTGAATCTGGTGGCGGTTTAGTTCAAGCCGGTGATAGCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCAATTCTGTTAAAGGTCGCTTCACTATTTCACGCGATGATGCGCGTAATACCGTGTACTTACAGATGAACTCACTGAAACCTGAGGAT
ACCGCCGTTTATTATTGTGCGGCAACGGCTATTAGCCCTATCTCTATTGTGGATGAGTATTGGGGTCAGGGCACACAAGTCACGGTCTCAAGCGGCGGAAG
CTCTGGTGAACAGAAACTGATTTCAGAAG
AIVHVFA#LDNDA#TIFMDSDLG#evqlvvsggglvqagdslrlscaasgAIVHVFAmgwfrqapgkerefvasinLDNDAtnyadsvkgrftisrddarntvylqmnslkpedtavyycna
TIFMDSDLGdywgqgtqvtvss#GGAGATATACCATGGAAGTTCAACTGGTTGTATCTGGTGGCGGTTTAGTTCAAGCCGGTGATAGCCTTCGTCTGAGCTGCG
CCGCAAGCGGAGCCATTGTGCATGTGTTCGCTATGGGTTGGTTTCGCCAGGCACCTGGTAAAGAACGTGAATTTGTGGCATCCATTAACCTTGACAACGACG
CTACCAACTATGCCGATTCTGTTAAAGGTCGCTTCACTATTTCACGCGATGATGCGCGTAATACCGTGTACTTACAGATGAACTCACTGAAACCTGAGGATAC
CGCCGTTTATTATTGTAATGCAACCATCTTTATGGATTCCGATTTGGGTGACTATTGGGGTCAGGGCACACAAGTCACGGTCTCAAGCGGCGGAAGCTCTGG
TGAACAGAAACTGATTTCAGAAG
AIVHVFA#LDNDA#TIFMDSDLG#qvqlvesggglvqagdslrlscaasgAIVHVFAmgwfrqapgkerefvasinLDNDAtnyadsvkgrftisrddarntvylqmnslkpedtavyycn
aTIFMDSDLGdywgqdtqvtvss#AGATATACCATGCAGGTTCAACTGGTTGAATCTGGTGGCGGTTTAGTTCAAGCCGGTGATAGCCTTCGTCTGAGCTGCGCC
GCAAGCGGAGCCATTGTGCATGTGTTCGCTATGGGTTGGTTTCGCCAGGCACCTGGTAAAGAACGTGAATTTGTGGCATCCATTAACCTTGACAACGACGCT
ACCAACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTAATGCAACCATCTTTATGGATTCCGATTTGGGTGACTATTGGGGTCAGGACACACAAGTCACGGTCTCAAGCGGCGGAAGCTCTGGTG
AACAGAAACTGATTTCAGAAG
AIVHVFA#LDNDA#TAISPISIVD#evqlvesggglvqagdslrlscaasgAIVHVFAmgwfrqapgkerefvasinLDNDAtnyadsvkgrftisrddarntvylqmnslkpedtavyycaa
TAISPISIVDeywgqgtrvtvss#AGGAGATATACCATGGAAGTTCAACTGGTTGAATCTGGTGGCGGTTTAGTTCAAGCCGGTGATAGCCTTCGTCTGAGCTGCG
CCGCAAGCGGAGCCATTGTGCATGTGTTCGCTATGGGTTGGTTTCGCCAGGCACCTGGTAAAGAACGTGAATTTGTGGCATCCATTAACCTTGACAACGACG
CTACCAACTATGCCGATTCTGTTAAAGGTCGCTTCACTATTTCACGCGATGATGCGCGTAATACCGTGTACTTACAGATGAACTCACTGAAACCTGAGGATAC
CGCCGTTTATTATTGTGCGGCAACGGCTATTAGCCCTATCTCTATTGTGGATGAGTATTGGGGTCAGGGCACACGAGTCACGGTCTCAAGCGGCGGAAGCTC
TGGTGAACAGAAACTGATTTCAGAAG
AIVHVFA#LDNDA#PFWINSSVV#evqlvesggglvqagdslrlscaasgAIVHVFAmgwfrqapgkehefvasinLDNDAtnyadsvkgrftisrddarntvylqmnslkpedtavyycn
aPFWINSSVVdywgqgtqvtvss#AAGGAGATATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACATGAATTTGTGGCATCCATTAACCTTGACAACG
ACGCTACCAACTATGCCGATTCTGTTAAAGGTCGCTTCACTATTTCACGCGATGATGCGCGTAATACCGTGTACTTACAGATGAACTCACTGAAACCTGAGGA
TACCGCCGTTTATTATTGTAATGCACCCTTCTGGATCAATAGTTCTGTTGTGGACTATTGGGGTCAGGGCACACAAGTCACGGTCTCAAGCGGCGGAAGCTC
TGGTGAACAGAAACTGATTTCAGAAG
AIVHVFA#LDNDA#ACSDYTYGSHGVR#evqlvesggglvqagdslrlscaasgAIVHVFAmgwfrqapgkerefvasinLDNDAtnyadsvkgrftisrddarntvylqmnslkpedtav
yycnaACSDYTYGSHGVRaswgqgtqvtvss#ATATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTAATGCAGCGTGTTCGGACTATACGTATGGCTCTCATGGTGTTCGGGCCTCTTGGGGTCAGGGCACACAAGTCACGGTCTCA
AGCGGCGGAAGCTCTGGTGAACAGAAACTGATTTCAGAAG
AIVHVFA#LDNDA#TIFMDSDLG#evqlvesggglvkagdslrlscaasgAIVHVFAmgwfrqapgkerefvasinLDNDAtnyadsvkgrftisrddarntvylqmnslkpedtavyycna
TIFMDSDLGdywgqgtqvtvss#AGGAGATATACCATGGAAGTTCAACTGGTTGAATCTGGTGGCGGTTTAGTTAAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTAATGCAACCATCTTTATGGATTCCGATTTGGGTGACTATTGGGGTCAGGGCACACAAGTCACGGTCTCAAGCGGCGGAAGCTCTG
GTGAACAGAAACTGATTTCAGAAGCCAA
AIVHVFA#LDNDA#WSFTPSICTL#qvqlqesggglvqagdslrlscaasgAIVHVFAmgwfrqapgkerefvasinLDNDAtnyadsvkgrftisrddarntvylqmnslkpedtavyyca
aWSFTPSICTLeywgqgtqvtvss#ATATACCATGCAGGTTCAACTGCAAGAATCTGGTGGCGGTTTAGTTCAAGCCGGTGATAGCCTTCGTCTGAGCTGCGCCG
CAAGCGGAGCCATTGTGCATGTGTTCGCTATGGGTTGGTTTCGCCAGGCACCTGGTAAAGAACGTGAATTTGTGGCATCCATTAACCTTGACAACGACGCTA
CCAACTATGCCGATTCTGTTAAAGGTCGCTTCACTATTTCACGCGATGATGCGCGTAATACCGTGTACTTACAGATGAACTCACTGAAACCTGAGGATACCGC
CGTTTATTATTGTGCGGCATGGAGTTTCACCCCTTCTATCTGTACCTTGGAGTATTGGGGTCAGGGCACACAAGTCACGGTCTCAAGCGGCGGAAGCTCTGG
TGAACAGAAACTGATTTCAGAAGCCAA
AIVHVFA#LDNDA#TIFMDSDLG#evqlvesggglvqagdslrlscaasgAIVHVFAmgwfrqapgkerefvasinLDNDAtnyadsvkgrftiscddarntvylqmnslkpedtavyycn
aTIFMDSDLGdywgqgtqvtvss#AGATATACCATGGAAGTTCAACTGGTTGAATCTGGTGGCGGTTTAGTTCAAGCCGGTGATAGCCTTCGTCTGAGCTGCGCC
GCAAGCGGAGCCATTGTGCATGTGTTCGCTATGGGTTGGTTTCGCCAGGCACCTGGTAAAGAACGTGAATTTGTGGCATCCATTAACCTTGACAACGACGCT
ACCAACTATGCCGATTCTGTTAAAGGTCGCTTCACTATTTCATGCGATGATGCGCGTAATACCGTGTACTTACAGATGAACTCACTGAAACCTGAGGATACCG
CCGTTTATTATTGTAATGCAACCATCTTTATGGATTCCGATTTGGGTGACTATTGGGGTCAGGGCACACAAGTCACGGTCTCAAGCGGCGGAAGCTCTGGTG
AACAGAAACTGATTTCAGAAGCCAA
AIVHVFA#LDNDA#PFWINSSVV#evqlvesggglvqagdslrlscaasgAIVHVFAmgwfrqapgkerefvasinLDNDAtnyadsvkgrftisredarntvylqmnslkpedtavyycna
PFWINSSVVdywgqgtqvtvss#AGGAGATATACCATGGAAGTTCAACTGGTTGAATCTGGTGGCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGAGGATGCGCGTAATACCGTGTACTTACAGATGAACTCACTGAAACCTGAGGAT
ACCGCCGTTTATTATTGTAATGCACCCTTCTGGATCAATAGTTCTGTTGTGGACTATTGGGGTCAGGGCACACAAGTCACGGTCTCAAGCGGCGGAAGCTCT
GGTGAACAGAAACTGATTTCAGAAGCCAA
AIVHVFA#LDNDA#TIFMDSDLG#evqlvesggglvqagdslrlscaasgAIVHVFAmgwfrqapgkerefvasinLDNDAtnyadsvkgrftisrddarntvylqInslkpedtavyycnaT
IFMDSDLGdywgqgtqvtvss#AGGAGATATACCATGGAAGTTCAACTGGTTGAATCTGGTGGCGGTTTAGTTCAAGCCGGTGATAGCCTTCGTCTGAGCTGCG
CCGCAAGCGGAGCCATTGTGCATGTGTTCGCTATGGGTTGGTTTCGCCAGGCACCTGGTAAAGAACGTGAATTTGTGGCATCCATTAACCTTGACAACGACG
CTACCAACTATGCCGATTCTGTTAAAGGTCGCTTCACTATTTCACGCGATGATGCGCGTAATACCGTGTACTTACAGTTGAACTCACTGAAACCTGAGGATAC
CGCCGTTTATTATTGTAATGCAACCATCTTTATGGATTCCGATTTGGGTGACTATTGGGGTCAGGGCACACAAGTCACGGTCTCAAGCGGCGGAAGCTCTGG
TGAACAGAAACTGATTTCAGAAGCCAA
AIVHVFA#LDNDA#TIFMDSHLG#evqlvesggglvqagdslrlscaasgAIVHVFAmgwfrqapgkerefvasinLDNDAtnyadsvkgrftisrddarntvylqmnslkpedtavyycna
TIFMDSHLGdywgqgtqvtvss#ACCATGGAAGTTCAACTGGTTGAATCTGGTGGCGGTTTAGTTCAAGCCGGTGATAGCCTTCGTCTGAGCTGCGCCGCAAGC
GGAGCCATTGTGCATGTGTTCGCTATGGGTTGGTTTCGCCAGGCACCTGGTAAAGAACGTGAATTTGTGGCATCCATTAACCTTGACAACGACGCTACCAAC
TATGCCGATTCTGTTAAAGGTCGCTTCACTATTTCACGCGATGATGCGCGTAATACCGTGTACTTACAGATGAACTCACTGAAACCTGAGGATACCGCCGTTT
ATTATTGTAATGCAACCATCTTTATGGATTCCCATTTGGGTGACTATTGGGGTCAGGGCACACAAGTCACGGTCTCAAGCGGCGGAAGCTCTGGTGAACAGA
AACTGATTTCAGAAGCCAA
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavyycna
TIFMDSDLGdywgqgtqgtvss#ATACCATGGAAGTTCAACTGGTTGAATCTGGTGGCGGTTTAGTTCAAGCCGGTGATAGCCTTCGTCTGAGCTGCGCCGCAA
GCGGAGCCATTGTGCATGTGTTCGCTATGGGTTGGTTTCGCCAGGCACCTGGTAAAGAACGTGAATTTGTGGCATCCATTAACCTTGACAACGACGCTACCA
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTAATGCAACCATCTTTATGGATTCCGATTTGGGTGACTATTGGGGTCAGGGCACACAAGGCACGGTCTCAAGCGGCGGAAGCTCTGGTGAACA
GAAACTGATTTCAGAAGCCAA
AIVHVFA#LDNDA#PFWINSSVV#evqlvesggglvqvgdslrlscaasgAIVHVFAmgwfrqapgkerefvasinLDNDAtnyadsvkgrftisrddahntvylqmnslkpedtavyycn
aPFWINSSVVdywgqgtqvtvss#ACCATGGAAGTTCAACTGGTTGAATCTGGTGGCGGTTTAGTTCAAGTCGGTGATAGCCTTCGTCTGAGCTGCGCCGCAAG
CGGAGCCATTGTGCATGTGTTCGCTATGGGTTGGTTTCGCCAGGCACCTGGTAAAGAACGTGAATTTGTGGCATCCATTAACCTTGACAACGACGCTACCAA
CTATGCCGATTCTGTTAAAGGTCGCTTCACTATTTCACGTGATGATGCGCATAATACCGTGTACTTACAGATGAACTCACTGAAACCTGAGGATACCGCCGTT
TATTATTGTAATGCACCCTTCTGGATCAATAGTTCTGTTGTGGACTATTGGGGTCAGGGCACACAAGTCACGGTCTCAAGCGGCGGAAGCTCTGGTGAACAG
AAACTGATTTCAGAAGCCAA
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtavhycn
aTIFMDSDLGdywgqgtqvtvss#ATACCATGGAAGTTCAACTGGTTGAATCTGGTGGCGGTTTAGTTCAAGCCGGTGATAGCCTTCGTCTGAGCTGCGCCGCA
AGCGGAGCCATTGTGCATGTGTTCGCTATGGGTTGGTTTCGCCAGGCACCTGGTAAAGAACGTGAATTTGTGGCATCCATTAACCTTGACAACGACGCTACC
AACTATGCCGATTCTGTTAAAGGTCGCTTCACTATTTCACGCGATGATGCGCGTAATACCGTGTACTTACAGATGAACTCACTGAAACCTGAGGATACCGCC
GTTCATTATTGTAATGCAACCATCTTTATGGATTCCGATTTGGGTGACTATTGGGGTCAGGGCACACAAGTCACGGTCTCAAGCGGCGGAAGCTCTGGTGAA
CAGAAACTGATTTCAGAAGCCAA
AIVHVFA#LDNDA#TIFMDSDLG#evqlvesggglvqagdslrlscaasgAIVHVFAmgwfrqapgkerefvasinLDNDAtnyvdsvkgrftisrddarntvylqmnslkpedtavyycna
TIFMDSDLGdywgqgtqvtvss#AAGGAGATATACCATGGAAGTTCAACTGGTTGAATCTGGTGGCGGTTTAGTTCAAGCCGGTGATAGCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGTCGATTCTGTTAAAGGTCGCTTCACTATTTCACGCGATGATGCGCGTAATACCGTGTACTTACAGATGAACTCACTGAAACCTGAGGAT
ACCGCCGTTTATTATTGTAATGCAACCATCTTTATGGATTCCGATTTGGGTGACTATTGGGGTCAGGGCACACAAGTCACGGTCTCAAGCGGCGGAAGCTCT
GGTGAACAGAAACTGATTTCAGAAGCCAA
AIVHVFA#LDNDA#PFWINSSVV#evqlvesggglvqagdslrlscaasgAIVHVFAmawfrqapgkerefvasinLDNDAtnyadsvkgrftisrddahntvylqmnslkpedtavyycn
aPFWINSSVVdywgqgtqvtvss#ATATACCATGGAAGTTCAACTGGTTGAATCTGGTGGCGGTTTAGTTCAAGCCGGTGATAGCCTTCGTCTGAGCTGCGCCGC
AAGCGGAGCCATTGTGCATGTGTTCGCTATGGCTTGGTTTCGCCAGGCACCTGGTAAAGAACGTGAATTTGTGGCATCCATTAACCTTGACAACGACGCTAC
CAACTATGCCGATTCTGTTAAAGGTCGCTTCACTATTTCACGCGATGATGCGCATAATACCGTGTACTTACAGATGAACTCACTGAAACCTGAGGATACCGCC
GTTTATTATTGTAATGCACCCTTCTGGATCAATAGTTCTGTTGTGGACTATTGGGGTCAGGGCACACAAGTCACAGTCTCAAGCGGCGGAAGCTCTGGTGAA
CAGAAACTGATTTCAGAAGCCAA
AIVHVFA#LDNDA#PFWINSSVV#evqlvesggglvqaggsIrlscaasgAIVHVFAmgwfrqapgkerefvasinLDNDAtnyadsvkgrftisrddahntvylqmnslkpedtavyycn
aPFWINSSVVdywgqgtqvtvss#GAAGGAGATATACCATGGAAGTTCAACTGGTTGAATCTGGTGGCGGTTTAGTTCAAGCCGGTGGTAGCCTTCGTCTGAGC
TGCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCATAATACCGTGTACTTACAGATGAACTCACTGAAACCTGAGG
ATACCGCCGTTTATTATTGTAATGCACCCTTCTGGATCAATAGTTCTGTTGTGGACTATTGGGGTCAGGGCACACAAGTCACGGTCTCAAGCGGCGGAAGCT
CTGGTGAACAGAAACTGATTTCAGAAGCCAA
AIVHVFA#LDNDA#TIFMDSDLG#evqlvesggglvqagdrlrlscaasgAIVHVFAmgwfrqapgkerefvasinLDNDAtnyadsvkgrftisrddarntvylqmnslkpedtavyycna
TIFMDSDLGdywgqgtqvtvss#GAAGGAGATATACCATGGAAGTTCAACTGGTTGAATCTGGTGGCGGTTTAGTTCAAGCCGGTGATCGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTAATGCAACCATCTTTATGGATTCCGATTTGGGTGACTATTGGGGTCAGGGCACACAAGTCACGGTCTCAAGCGGCGGAAGC
TCTGGTGAACAGAAACTGATTTCAGAAGCCAA
PIVHVFA#LDNDA#TIFMDSDLG#evqlvesggglvqagdslrlscaasgPIVHVFAmgwfrqapgkerefvasinLDNDAtnyadsvkgrftisrddarntvylqmnslkpedtavyycna
TIFMDSDLGdywgqgtqvtvss#GAATGCGATATACCATGGAAGTTCAACTGGTTGAATCTGGTGGCGGTTTAGTTCAAGCCGGTGATAGCCTTCGTCTGAGCT
GCGCCGCAAGCGGACCCATTGTGCATGTGTTCGCTATGGGTTGGTTTCGCCAGGCACCTGGTAAAGAACGTGAATTTGTGGCATCCATTAACCTTGACAACG
ACGCTACCAACTATGCCGATTCTGTTAAAGGTCGCTTCACTATTTCACGCGATGATGCGCGTAATACCGTGTACTTACAGATGAACTCACTGAAACCTGAGGA
TACCGCCGTTTATTATTGTAATGCAACCATCTTTATGGATTCCGATTTGGGTGACTATTGGGGTCAGGGCACACAAGTCACGGTCTCAAGCGGCGGAAGCTC
TGGTGAACAGAAACTGATTTCAGAAGCCAA
AIVHVFA#LDNDA#TIFMDSDLG#evqlvesggglvqagdslrlscatsgAIVHVFAmgwfrqapgkerefvasinLDNDAtnyadsvkgrftisrddarntvylqmnslkpedtavyycna
TIFMDSDLGdywgqgtqvtvss#ACCATGGAAGTTCAACTGGTTGAATCTGGTGGTGGTTTAGTTCAAGCCGGTGATAGCCTTCGTCTGAGCTGCGCCACAAGC
GGAGCCATTGTGCATGTGTTCGCTATGGGTTGGTTTCGCCAGGCACCTGGTAAAGAACGTGAATTTGTGGCATCCATTAACCTTGACAACGACGCTACCAAC
TATGCCGATTCTGTTAAAGGTCGCTTCACTATTTCACGCGATGATGCGCGTAATACCGTGTACTTACAGATGAACTCACTGAAACCTGAGGATACCGCCGTTT
ATTATTGTAATGCAACCATCTTTATGGATTCCGATTTGGGTGACTATTGGGGTCAGGGCACACAAGTCACGGTCTCAAGCGGCGGAAGCTCTGGTGAACAG
AAACTGATTTCAGAAGCCAA
AIVHVFA#LDNDA#TLYHTIREAR#evqlvesggglvqagdslrlscaasgAIVHVFAmgwfrqapgkerefvasinLDNDAtnyadsvkgrftisrddarntvylqmnslkpedtavyycat
TLYHTIREARdywgqgtqvtvss#AGGAGATATACCATGGAAGTTCAACTGGTTGAATCTGGTGGCGGTTTAGTTCAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTGCGACAACTCTGTACCATACGATTCGGGAGGCCCGCGACTATTGGGGTCAGGGCACACAAGTCACGGTCTCAAGCGGCGGAAGC
TCTGGTGAACAGAAACTGATTTCAGAAGCCAA
AIVHVFA#LDNDA#PAYHLASMVT#evqlfesggglvqagdslrlscaasgAIVHVFAmgwfrqapgkerefvasinLDNDAtnyadsvkgrftisrddarntvylqmnslkpedtavyyc
aaPAYHLASMVTdywgqgtqvtvss#ATATACCATGGAAGTTCAACTGTTTGAATCTGGTGGCGGTTTAGTTCAAGCCGGTGATAGCCTTCGTCTGAGCTGCGCC
GCAAGCGGAGCCATTGTGCATGTGTTCGCTATGGGTTGGTTTCGCCAGGCACCTGGTAAAGAACGTGAATTTGTGGCATCCATTAACCTTGACAACGACGCT
ACCAACTATGCCGATTCTGTTAAAGGTCGCTTCACTATTTCACGCGATGATGCGCGTAATACCGTGTACTTACAGATGAACTCACTGAAACCTGAGGATACCG
CCGTTTATTATTGTGCGGCACCTGCCTACCATCTCGCCTCTATGGTTACCGACTATTGGGGTCAGGGCACACAAGTCACGGTCTCAAGCGGCGGAAGCTCTG
GTGAACAGAAACTGATTTCAGAAGCCAA
AIVNVFA#LDNDA#TIFMDSDLG#evqlvesggglvqagdslrlscaasgAIVNVFAmgwfrqapgkerefvasinLDNDAtnyadsvkgrftisrddarntvylqmnslkpedtavyycn
aTIFMDSDLGdywgqgtqvtvss#ATATACCATGGAAGTTCAACTGGTTGAATCTGGTGGCGGTTTAGTTCAAGCCGGTGATAGCCTTCGTCTGAGCTGCGCCGC
AAGCGGAGCCATTGTGAATGTGTTCGCTATGGGTTGGTTTCGCCAGGCACCTGGTAAAGAACGTGAATTTGTGGCATCCATTAACCTTGACAACGACGCTAC
CAACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTAATGCAACCATCTTTATGGATTCCGATTTGGGTGACTATTGGGGTCAGGGCACACAAGTCACGGTCTCAAGCGGCGGAAGCTCTGGTGAA
CAGAAACTGATTTCAGAAGCCAA
AIVHVFA#LDNDA#TAISPISIVD#evqlvesggglvqagdslrlscaasgAIVHVFAmgwfrqapgkerefvasinLDNDAtnyadsikgrftisrddarntvylqmnslkpedtavyycaaT
AISPISIVDeywgqgtqvtvss#AGATATACCATGGAAGTTCAACTGGTTGAATCTGGTGGCGGTTTAGTTCAAGCCGGGGATAGCCTTCGTCTGAGCTGCGCCGC
AAGCGGAGCCATTGTGCATGTGTTCGCTATGGGTTGGTTTCGCCAGGCACCTGGTAAAGAACGTGAATTTGTGGCATCCATTAACCTTGACAACGACGCTAC
CAACTATGCCGATTCTATTAAAGGTCGCTTCACTATTTCACGCGATGATGCGCGTAATACCGTGTACTTACAGATGAACTCACTGAAACCTGAGGATACCGCC
GTTTATTATTGTGCGGCAACGGCTATTAGCCCTATCTCTATTGTGGATGAGTATTGGGGTCAGGGCACACAAGTCACGGTCTCAAGCGGCGGAAGCTCTGGT
GAACAGAAACTGATTTCAGAAGCCAA
AIVHVFA#LDNDA#TIFMDSDLG#evqlvesggglvqvgdslrlscaasgAIVHVFAmgwfrqapgkerefvasinLDNDAtnyadsvkgrftisrddarntvylqmnslkpedtavyycna
TIFMDSDLGdywgqgtqvtvss#ACCATGGAAGTTCAACTGGTTGAATCTGGTGGCGGTTTAGTTCAAGTCGGTGATAGCCTTCGTCTGAGCTGCGCCGCAAGC
GGAGCCATTGTGCATGTGTTCGCTATGGGTTGGTTTCGCCAGGCACCTGGTAAAGAACGTGAATTTGTGGCATCCATTAACCTTGACAACGACGCTACCAAC
TATGCCGATTCTGTTAAAGGTCGCTTCACTATTTCACGCGATGATGCGCGTAATACCGTGTACTTACAGATGAACTCACTGAAACCTGAGGATACCGCCGTTT
ATTATTGTAATGCAACCATCTTTATGGATTCCGATTTGGGTGACTATTGGGGTCAGGGCACACAAGTCACGGTCTCAAGCGGCGGAAGCTCTGGTGAACAG
AAACTGATTTCAGAAGCCAA
AIVHVFA#LDNDA#PFWINSSVV#evqlvesggglvqagdslrlscaasgAIVHVFAmgwfrqapgkerefvasinLDNDAsnyadsvkgrftisrddarntvylqmnslkpedtavyycn
aPFWINSSVVdywgqgtqvtvss#ATACCATGGAAGTTCAACTGGTTGAATCTGGTGGCGGTTTAGTTCAAGCCGGTGATAGCCTTCGTCTGAGCTGCGCCGCA
AGCGGAGCCATTGTGCATGTGTTCGCTATGGGTTGGTTTCGCCAGGCACCTGGTAAAGAACGTGAATTTGTGGCATCCATTAACCTTGACAACGACGCTTCC
AACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTAATGCACCCTTCTGGATCAATAGTTCTGTTGTGGACTATTGGGGTCAGGGCACACAAGTCACGGTCTCAAGCGGCGGAAGCTCTGGTGAA
CAGAAACTGATTTCAGAAGCCAA
AIVHVFA#LDNDA#TIFIDSDLG#evqlvesggglvqggdslrlscaasgAIVHVFAmgwfrqapgkerefvasinLDNDAtnyadsvkgrftisrddarntvylqmnslkpedtavyycnaT
IFIDSDLGdywgqgtqvtvss#ATATACCATGGAAGTTCAACTGGTTGAATCTGGTGGCGGTTTAGTTCAAGGCGGTGATAGCCTTCGTCTGAGCTGCGCCGCAA
GCGGAGCCATTGTGCATGTGTTCGCTATGGGTTGGTTTCGCCAGGCACCTGGTAAAGAACGTGAATTTGTGGCATCCATTAACCTTGACAACGACGCTACCA
ACTATGCCGATTCTGTTAAAGGTCGCTTCACTATTTCACGCGATGATGCACGTAATACCGTGTACTTACAGATGAACTCACTGAAACCTGAGGATACCGCCGT
TTATTATTGTAATGCAACCATCTTTATAGATTCCGATTTGGGTGACTATTGGGGTCAGGGCACACAAGTCACGGTCTCAAGCGGCGGAAGCTCTGGTGAACA
GAAACTGATTTCAGAAGCCAA
AIVHVFA#LDNDA#PFWINSSVV#evqlvesggglvhagdrlrlscaasgAIVHVFAmgwfrqapgkerefvasinLDNDAtnyadsvkgrftisrddahntvylqmnslkpedtavyycn
aPFWINSSVVdywgqgtqvtvss#GAAGGAGATATACCATGGAAGTTCAACTGGTTGAATCTGGTGGCGGTTTAGTTCACGCCGGTGATCGCCTTCGTCTGAGC
TGCGCCGCAAGCGGAGCCATTGTGCATGTGTTCGCTATGGGTTGGTTTCGCCAGGCACCCGGTAAAGAACGTGAATTTGTGGCATCCATTAACCTTGACAAC
GACGCTACCAACTATGCCGATTCTGTTAAAGGTCGCTTCACTATTTCACGCGATGATGCGCATAATACCGTGTACTTACAGATGAACTCACTGAAACCTGAGG
ATACCGCCGTTTATTATTGTAATGCACCCTTCTGGATCAATAGTTCTGTTGTGGACTATTGGGGTCAGGGCACACAAGTCACGGTCTCAAGCGGCGGAAGCT
CTGGTGAACAGAAACTGATTTCAGAAGCCAA
AIVHVFA#LDNDA#TIFMDSDLG#evqlvesgggliqagdslrlscaasgAIVHVFAmgwfrqapgkerefvasinLDNDAtnyadsvkgrftisrddarntvylqmnslkpedtavyycna
TIFMDSDLGdywgqgtqvtvss#GAAGGAGATATACCATGGAAGTTCAACTGGTTGAATCTGGTGGCGGTTTAATTCAAGCCGGTGATAGCCTACGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTAATGCAACCATCTTTATGGATTCCGATTTGGGTGACTATTGGGGTCAGGGCACACAAGTCACGGTCTCAAGCGGCGGAAGC
TCTGGTGAACAGAAACTGATTTCAGAAGCCAA
AIVHVFA#LDNDA#FCYVPLATC#evqlvesggglvqagdslrlscaasgAIVHVFAmgwfrqapgkerefvasinLDNDAtnyvdsvkgrftisrddarntvylqmnslkpedtavyycna
FCYVPLATCdylgqgtqvtvss#AGATATACCATGGAAGTTCAACTGGTTGAATCTGGTGGCGGTTTAGTTCAAGCCGGTGATAGCCTTCGTCTGAGCTGCGCCGC
AAGCGGAGCCATTGTGCATGTGTTCGCTATGGGTTGGTTTCGCCAGGCACCTGGTAAAGAACGTGAATTTGTGGCATCCATTAACCTTGACAACGACGCTAC
CAACTATGTCGATTCTGTTAAAGGTCGCTTCACTATTTCACGCGATGATGCGCGTAATACCGTGTACTTACAGATGAACTCACTGAAACCTGAGGATACCGCC
GTTTATTACTGTAATGCATTCTGCTACGTCCCTTTGGCCACGTGTGACTATTTGGGTCAGGGCACACAAGTCACGGTCTCAAGCGGCGGAAGCTCTGGTGAA
AAGAAACTGATTTCAGAAGCCAA
AIVHVFA#LDNDA#PFWINSSVV#evrlvesggglvqagdslrlscaasgAIVHVFAmgwfrqapgkerefvasinLDNDAtnyadsvkgrftisrddahntvylqmnslkpedtavyycn
aPFWINSSVVdywgqgtqvtvss#AGATATACCATGGAAGTTCGACTGGTTGAATCTGGTGGCGGTTTAGTTCAAGCCGGTGATAGCCTTCGTCTGAGCTGCGC
CGCAAGCGGAGCCATTGTGCATGTGTTCGCTATGGGTTGGTTTCGCCAGGCACCTGGTAAAGAACGTGAATTTGTGGCATCCATTAACCTTGACAACGACGC
TACCAACTATGCCGATTCTGTTAAAGGTCGCTTCACTATTTCACGCGATGATGCGCATAATACCGTGTACTTACAGATGAACTCACTGAAACCTGAGGATACC
GCCGTTTATTATTGTAATGCACCCTTCTGGATCAATAGTTCTGTTGTGGACTATTGGGGTCAGGGCACACAAGTCACGGTCTCAAGCGGCGGAAGCTCTGGT
GAACAGAAACTGATTTCAGAAGCCAA
AIVHVFA#LDNDA#TIFMDSDLG#evklvesggglvkagdslrlscaasgAIVHVFAmgwfrqapgkerefvasinLDNDAtnyadsvkgrftisrddarntvylqmnslkpedtavyycna
TIFMDSDLGdywgqgtqvtvss#AGGAGATATACCATGGAAGTTAAACTGGTTGAATCTGGTGGCGGTTTAGTTAAAGCCGGTGATAGCCTTCGTCTGAGCTGC
GCCGCAAGCGGAGCCATTGTGCATGTGTTCGCTATGGGTTGGTTTCGCCAGGCACCTGGTAAAGAACGTGAATTTGTGGCATCCATTAACCTTGACAACGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTATTGTAATGCAACCATCTTTATGGATTCCGATTTGGGTGACTATTGGGGTCAGGGCACACAAGTCACGGTCTCAAGCGGCGGAAGCTCTG
GTGAACAGAAACTGATTTCAGAAGCCAA
AIVHVFA#LDNDA#FCYVPLATC#evqlvesggglvqagdslrlscaasgAIVHVFAmgwfrqapgkerefvasinLDNDAtnyadsvkgrftisrddarntvylqmnslkpedtavyycna
FCYVPLATCdyrgqgtqvtvss#ACCATGGAAGTTCAACTGGTTGAATCTGGTGGCGGTTTAGTTCAAGCCGGTGATAGCCTTCGTCTGAGCTGTGCCGCAAGCG
GAGCCATTGTGCATGTGTTCGCTATGGGTTGGTTTCGCCAGGCACCTGGTAAAGAACGTGAATTTGTGGCATCCATTAACCTTGACAACGACGCTACCAACT
ATGCCGATTCTGTTAAAGGTCGCTTCACTATTTCACGCGATGATGCGCGTAATACCGTGTACTTACAGATGAACTCACTGAAACCTGAGGATACCGCCGTTTA
TTATTGTAATGCATTCTGCTACGTCCCTTTGGCCACGTGTGACTATAGGGGTCAGGGCACACAAGTCACGGTCTCAAGCGGCGGAAGCTCTGGTGAACAGA
AACTGATTTCAGAAGCCAA
AIVHVFA#LDNDA#FCYVPLATC#qvqlvesggglvqagdslrlscaasgAIVHVFAmgwfrqapgkerefvasinLDNDAtnyadsvkgrftisrddarntvylqmnslkpedtavyycna
FCYVPLATCdywgqgtqvtvss#ATATACCATGCAGGTTCAACTGGTTGAATCTGGTGGCGGTTTAGTTCAAGCCGGTGATAGCCTTCGTCTGAGCTGCGCCGCA
AGCGGAGCCATTGTGCATGTGTTCGCTATGGGTTGGTTTCGCCAGGCACCTGGTAAAGAACGTGAATTTGTGGCATCCATTAACCTTGACAACGACGCTACC
AACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTAATGCATTCTGCTACGTCCCTTTGGCCACGTGTGACTATTGGGGTCAGGGCACACAAGTCACGGTCTCAAGCGGCGGAAGCTCTGGTGAA
CAGAAACTGATTTCAGAAGCCAA
AIVHVFA#LDNDA#TIFMDSDLG#evqlvesggglvqagdslrlscaasgAIVHVFAmgcfrqapgkerefvasinLDNDAtnyadsvkgrftisrddarntvylqmnslkpedtavyycna
TIFMDSDLGdywvqgtqvtvss#ACCATGGAAGTTCAACTGGTTGAATCTGGTGGCGGTTTAGTTCAAGCCGGTGATAGCCTTCGTCTGAGCTGCGCCGCAAGC
GGAGCCATTGTGCATGTGTTCGCTATGGGTTGTTTTCGCCAGGCACCTGGTAAAGAACGTGAATTTGTGGCATCCATTAACCTTGACAACGACGCTACCAAC
TATGCCGATTCTGTTAAAGGTCGCTTCACTATTTCACGCGATGATGCGCGTAATACCGTGTACTTACAGATGAACTCACTGAAACCTGAGGATACCGCCGTTT
ATTATTGTAATGCAACCATCTTTATGGATTCCGATTTGGGTGACTATTGGGTTCAGGGCACACAAGTCACGGTCTCAAGCGGCGGAAGCTCTGGTGAAAAGA
AACTGATTTCAGAAGCCAA
AIVHVFA#LDNDA#TIFMDSDLG#evqlvesggglvqagdslrlscaasgAIVHVFAmgwfrqapgkerefvasinLDNDAtnyadsvkgrftisrddarntvylqmnslkpedtafyycna
TIFMDSDLGdywgqgtqvtvss#GAAGGAGATATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCTTTTTATTATTGTAATGCAACCATCTTTATGGATTCCGATTTGGGTGACTATTGGGGTCAGGGCACACAAGTCACGGTCTCAAGCGGCGGAAGC
TCTGGTGAACAGAAACTGATTTCAGAAGCCAA
AIVHVFA#LDNDA#PFWINSSVV#gvqlvesggglvqagdslrlscaasgAIVHVFAmgwfrqapgkerefvasinLDNDAtnyadsvkgrftisrddahntvylqmnslkpedtavyycn
aPFWINSSVVdywgqgtqvtvss#AGGAGATATACCATGGGAGTTCAACTGGTTGAATCTGGTGGCGGTTTAGTTCAAGCCGGTGATAGCCTTCGTCTGAGCTG
CGCCGCAAGCGGAGCCATTGTGCATGTGTTCGCTATGGGTTGGTTTCGCCAGGCACCTGGTAAAGAACGTGAATTTGTGGCATCCATTAACCTTGACAACGA
CGCTACCAACTATGCCGATTCTGTTAAAGGTCGCTTCACTATTTCACGCGATGATGCGCATAATACCGTGTACTTACAGATGAACTCACTGAAACCTGAGGAT
ACCGCCGTTTATTATTGTAATGCACCCTTCTGGATCAATAGTTCTGTTGTGGACTATTGGGGGCAGGGCACACAAGTCACGGTCTCAAGCGGCGGAAGCTCT
GGTGAACAGAAAATGATTTCAGAAGCCAA
AIVHVFA#LDNDA#TIFMDSDLG#evqlvesggglvqagdslrlscaasgAIVHVFAmgwfrqapgkerefvasidLDNDAtnyadsvkgrftisrddarntvylqmnslkpedtavyycna
TIFMDSDLGdywgqgtqvtvss#GAAGGAGATATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTGACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTAATGCAACCATCTTTATGGATTCCGATTTGGGTGACTATTGGGGTCAGGGCACACAAGTCACGGTCTCAAGCGGCGGAAGC
TCTGGTGAACAGAAACTGATTTCAGAAGCCAA
AIVLVFA#LDNDA#TIFMDSDLG#evqlvesggglvqagdslrlscaasgAIVLVFAmgwfrqapgkerefvasinLDNDAtnyadsvkgrftisrddarntvylqmnslkpedtavyycna
TIFMDSDLGdywgqgtqvtvss#ATACCATGGAAGTTCAACTGGTTGAATCTGGTGGCGGTTTAGTTCAAGCCGGTGATAGCCTTCGTCTGAGCTGCGCCGCAA
GCGGAGCCATTGTGCTTGTGTTCGCTATGGGTTGGTTTCGCCAGGCACCTGGTAAAGAACGTGAATTTGTGGCATCCATTAACCTTGACAACGACGCTACCA
ACTATGCCGATTCTGTTAAAGGTCGCTTTACTATTTCACGCGATGATGCGCGTAATACCGTGTACTTACAGATGAACTCACTGAAACCTGAGGATACCGCCGT
TTATTATTGTAATGCAACCATCTTTATGGATTCCGATTTGGGTGACTATTGGGGTCAGGGCACACAAGTCACGGTCTCAAGCGGCGGAAGCTCTGGTGAACA
GAAACTGATTTCAGAAGCCAA
AIVHVFA#LDNDA#WSFTPSICTL#evqlvesggglvqagdslrlscaasgAIVHVFAmgwfrqapgkerefvasinLDNDAtnyadsvkgrftisrddarntvylqmnslkpedtavyyca
aWSFTPSICTLeywgqgtqgtvss#AAGGAGATATACCATGGAAGTTCAACTGGTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTGCGGCATGGAGTTTCACCCCTTCTATCTGTACCTTGGAGTATTGGGGTCAGGGCACACAAGGCACGGTCTCAAGCGGCGGA
AGCTCTGGTGAACAGAAACTGATTTCAGAAGCCAA
AIVHVFA#LDNDA#TIFMDSDLG#evqlvesggglvqagdslrlscaasgAIVHVFAmdwfrqapgkerefvasinLDNDAtnyadsvkgrftisrddarntvylqmnslkpedtavyycn
aTIFMDSDLGdywgqgtqgtvss#ATACCATGGAAGTTCAACTGGTTGAATCTGGTGGCGGTTTAGTTCAAGCCGGTGATAGCCTTCGTCTGAGCTGCGCCGCA
AGCGGAGCCATTGTGCATGTGTTCGCTATGGATTGGTTTCGCCAGGCACCTGGTAAAGAACGTGAATTTGTGGCATCCATTAACCTTGACAACGACGCTACC
AACTATGCCGATTCTGTTAAAGGTCGCTTCACTATTTCACGCGATGATGCGCGTAATACCGTGTACTTACAGATGAACTCACTGAAACCTGAGGATACCGCC
GTTTATTATTGTAATGCAACCATCTTTATGGATTCCGATTTGGGTGACTATTGGGGTCAGGGCACACAAGGCACGGTCTCAAGCGGCGGAAGCTCTGGTGAA
CAGAAACTGATTTCAGAAGCCAA
AIVHVFA#LDNDA#TIFMDSDLG#qvqlvesggglvqagdslrlscaasgAIVHVFAmgwfrqapgkerefvasinLDNDAtnyadsvkgrftisrddarntvylqmnslkpedtavyycn
aTIFMDSDLGdywgqgtqgtvss#ATACCATGCAGGTTCAACTGGTTGAATCTGGTGGCGGTTTAGTTCAAGCCGGTGATAGCCTTCGTCTGAGCTGCGCCGCAA
GCGGAGCCATTGTGCATGTGTTCGCTATGGGTTGGTTTCGCCAGGCACCTGGTAAAGAACGTGAATTTGTGGCATCCATTAACCTTGACAACGACGCTACCA
ACTATGCCGATTCTGTTAAAGGTCGCTTCACTATTTCACGCGATGATGCGCGTAATACCGTGTACTTACAGATGAACTCACTGAAACCTGAGGATACCGCCGT
TTATTATTGTAATGCAACCATCTTTATGGATTCCGATTTGGGTGACTATTGGGGTCAGGGCACACAAGGCACGGTCTCAAGCGGCGGAAGCTCTGGTGAACA
GAAACTGATTTCAGAAGCCAA
AIVHVFA#LDNDA#PFWINSSVV#evqlvesggglvqagdslrlscaasgAIVHVFAmgwfrqapgkerefvasinLDNDAtnyadsvkgrftisrddahntvylqmnslkpedtavyycn
aPFWINSSVVdywgqgtqgtvss#GAAGGAGATATACCATGGAAGTTCAACTGGTTGAATCTGGTGGCGGTTTAGTTCAAGCCGGTGATAGCCTTCGTCTGAGC
TGCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCATAATACCGTGTACTTACAGATGAACTCACTGAAACCTGAGG
ATACCGCCGTTTATTATTGTAATGCACCCTTCTGGATCAATAGTTCTGTTGTGGACTATTGGGGTCAGGGCACACAAGGCACGGTCTCAAGCGGCGGAAGCT
CTGGTGAACAGAAACTGATTTCAGAAGCCAA
AIVHVFA#LDNDA#PFWINSSVV#evqlvdsggglvqagdrlrlscaasgAIVHVFAmgwfrqapgkerefvasinLDNDAtnyadsvkgrftisrddahntvylqmnslkpedtavyycn
aPFWINSSVVdywgqgtqgtvss#GAAGGAGATATACCATGGAAGTTCAACTGGTTGACTCTGGTGGCGGTTTAGTTCAAGCCGGTGATCGCCTTCGTCTGAGC
TGCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCATAATACCGTGTACTTACAGATGAACTCACTGAAACCTGAGG
ATACCGCCGTTTATTATTGTAATGCACCCTTCTGGATCAATAGTTCTGTTGTGGACTATTGGGGTCAGGGCACACAAGGCACGGTCTCAAGCGGCGGAAGCT
CTGGTGAACAGAAACTGATTTCAGAAGCCAA
AIVHVFA#LDNDA#ACSDYTYGSHGVR#evqlvesggglvqagdslrlscaasgAIVHVFAmgwfrqapgkerefvasinLDNDAtnyadsvkgrftisrddarntvylqmnslkpedtav
yycnaACSDYTYGSHGVRdywgqgtqgtvss#GGAGATATACCATGGAAGTTCAACTGGTTGAATCTGGTGGCGGTTTAGTTCAAGCCGGTGATAGCCTTCGTCT
GAGCTGCGCCGCAAGCGGAGCCATTGTGCATGTGTTCGCTATGGGTTGGTTTCGCCAGGCACCTGGTAAAGAACGTGAATTTGTGGCATCCATTAACCTTG
ACAACGACGCTACCAACTATGCCGATTCTGTTAAAGGTCGCTTCACTATTTCACGCGATGATGCGCGTAATACCGTGTACTTACAGATGAACTCACTGAAACC
TGAGGATACCGCCGTTTATTATTGTAATGCAGCGTGTTCGGACTATACGTATGGCTCTCATGGTGTTCGGGACTATTGGGGTCAGGGCACACAAGGCACTGT
CTCAAGCGGCGGAAGCTCTGGTGAACAGAAACTGATTTCAGAAGCCAA
AIVHVFA#LDNDA#ACSDYTYGSHGVR#evqlvesggglvqagdslrlscaasgAIVHVFAmgwfrqapgkerefvasinLDNDAtnyadsvkgrftisrddarntvylqmnslkpedtav
yycnaACSDYTYGSHGVRdswgqgtqgtvss#GGAGATATACCATGGAAGTTCAACTGGTTGAATCTGGTGGCGGTTTAGTTCAAGCCGGTGATAGTCTTCGTCT
GAGCTGCGCCGCAAGCGGAGCCATTGTGCATGTGTTCGCTATGGGTTGGTTTCGCCAGGCACCTGGTAAAGAACGTGAATTTGTGGCATCCATTAACCTTG
ACAACGACGCTACCAACTATGCCGATTCTGTTAAAGGTCGCTTCACTATTTCACGCGATGATGCGCGTAATACCGTGTACTTACAGATGAACTCACTGAAACC
TGAGGATACCGCCGTTTATTATTGTAATGCAGCGTGTTCGGACTATACGTATGGCTCTCATGGTGTTCGGGACTCTTGGGGTCAGGGCACACAAGGCACGG
TCTCAAGCGGCGGAAGCTCTGGTGAACAGAAACTGATTTCAGAAGCCAA
AIVHVFA#LDNDA#PFWINSSVV#qvqlvesggglvqagdslrlscaasgAIVHVFAmgwfrqapgkerefvasinLDNDAtnyadsvkgrftisrddahntvylqmnspkpedtavyyc
naPFWINSSVVdywgqgtqvtvss#AAGGAGATATACCATGCAGGTTCAACTGGTTGAATCTGGTGGCGGTTTAGTTCAAGCCGGTGATAGCCTTCGTCTGAGC
TGCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCATAATACCGTGTACTTACAGATGAACTCACCGAAACCTGAG
GATACCGCCGTTTATTATTGTAATGCACCCTTCTGGATCAATAGTTCTGTTGTGGACTATTGGGGTCAGGGCACACAAGTCACGGTCTCAAGCGGCGGAAGC
TCTGGTGAACAGAAACTGATTTCAGAAGCCAA
AIVHVFA#LDNDA#ACSDYTYGSHGVR#evqlfesggglvqagdslrlscaasgAIVHVFAmgwfrqapgkerefvasinLDNDAtnyadsvkgrftisrddarntvylqmnslkpedtav
yycnaACSDYTYGSHGVRdswgqgtqvtvss#ATATACCATGGAAGTTCAACTGTTTGAATCTGGTGGCGGTTTAGTTCAAGCCGGTGATAGCCTTCGTCTGAGCT
GCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTAATGCAGCGTGTTCGGACTATACGTATGGCTCTCATGGTGTTCGGGACTCTTGGGGTCAGGGCACACAAGTCACGGTCTCA
AGCGGCGGAAGCTCTGGTGAACAGAAACTGATTTCAGAAGCCAA
AIVHVFA#LDNDA#FCYVPLATC#evqlvesggglvqagdslrlscaasgAIVHVFAmgwfrqapgkereivasinLDNDAtnyadsvkgrftisrddarntvylqmnslkpedtavyycna
FCYVPLATCdylgqgtqvtvss#AGGAGATATACCATGGAAGTTCAACTGGTTGAATCTGGTGGCGGTTTAGTTCAAGCCGGTGATAGCCTTCGTCTGAGCTGCG
CCGCAAGCGGAGCCATTGTGCATGTGTTCGCTATGGGTTGGTTTCGCCAGGCACCTGGTAAAGAACGTGAAATTGTGGCATCCATTAACCTTGACAACGAC
GCTACCAACTATGCCGATTCTGTTAAAGGTCGCTTCACTATTTCACGCGATGATGCGCGTAATACCGTGTACTTACAGATGAACTCACTGAAACCTGAGGATA
CCGCCGTTTATTACTGTAATGCATTCTGCTACGTCCCTTTGGCCACGTGTGACTATTTGGGTCAGGGCACACAAGTCACGGTCTCAAGCGGCGGAAGCTCTG
GTGAACAGAAACTGATTTCAGAAGCCAA
AIVHVFA#LDNDA#PFWINSSVV#evqlvesggglvqagdslrlscaasgAIVHVFAmgwfrqapgkerefvasinLDNDAtnyadsvkgrftisrddahntvylqmnslkpedtavyycn
aPFWINSSVVdywgqgtqvavss#ACCATGGAAGTTCAACTGGTTGAATCTGGTGGCGGTTTAGTTCAAGCCGGTGATAGCCTTCGTCTGAGCTGCGCCGCAAG
CGGAGCCATTGTGCATGTGTTCGCTATGGGTTGGTTTCGTCAGGCACCTGGTAAAGAACGTGAATTTGTGGCATCCATTAACCTTGACAACGACGCTACCAA
CTATGCCGATTCTGTTAAAGGTCGCTTCACTATTTCACGCGATGATGCGCATAATACCGTGTACTTACAGATGAACTCACTGAAACCTGAGGATACCGCCGTT
TATTATTGTAATGCACCCTTCTGGATCAATAGTTCTGTTGTGGACTATTGGGGTCAGGGCACACAAGTCGCGGTCTCAAGCGGCGGAAGCTCTGGTGAACAG
AAACTGATTTCAGAAGCCAA
AIVHVFA#LDNDA#TIFMDSDSG#evqlvesggglvqagdslrlscaasgAIVHVFAmgwfrqapgkerefvasinLDNDAtnyadsvkgrftisrddarntvylqmnslkpedtavyycn
aTIFMDSDSGdywgqdtqgtvss#GAAGGAGATATACCATGGAAGTTCAACTGGTTGAATCTGGTGGCGGTTTAGTTCAAGCCGGTGATAGCCTTCGTCTGAGC
TGCGCCGCAAGCGGAGCCATTGTGCATGTGTTCGCTATGGGTTGGTTTCGCCAGGCACCTGGTAAAGAACGTGAATTTGTGGCATCCATTAACCTTGACAAC
GACGCTACCAACTATGCCGATTCTGTTAAAGGTCGCTTCACTATTTCACGCGATGATGCGCGTAATACCGTGTACTTACAGATGAACTCACTGAAACCTGAG
GATACCGCCGTTTATTATTGTAATGCAACCATCTTTATGGATTCCGATTCGGGTGACTATTGGGGTCAGGACACACAAGGCACGGTCTCAAGCGGCGGAAGC
TCTGGTGAACAGAAAATGATTTCAGAAGCCAA
AIVHVFA#LDNDA#FCYVPLATC#evqlvesggglvqagdslrlscaasgAIVHVFAmgwfrqapgkerefvasinLDNDAtnyadsvkgrftisrddarntvylqmnslkpedtavyycna
FCYVPLATCdywgqgtqgtvss#GGAGATATACCATGGAAGTTCAACTGGTTGAATCTGGTGGCGGTTTAGTTCAAGCCGGTGATAGCCTTCGTCTGAGCTGCG
CCGCAAGCGGAGCCATTGTGCATGTGTTCGCTATGGGTTGGTTTCGCCAGGCACCTGGTAAAGAACGTGAATTTGTGGCATCCATTAACCTTGACAACGACG
CTACCAACTATGCCGATTCTGTTAAAGGTCGCTTCACTATTTCACGCGATGATGCGCGTAATACCGTGTACTTACAGATGAACTCACTGAAACCTGAGGATAC
CGCCGTTTATTATTGTAATGCATTCTGCTACGTCCCTTTGGCCACGTGTGACTATTGGGGTCAGGGCACACAAGGCACGGTCTCAAGCGGCGGAAGCTCTGG
TGAACAGAAAATGATTTCAGAAGCCAA
AIVHVFA#LDNDA#TAISPISIVD#kvqlvesggglvqagdslrlscaasgAIVHVFAmgwfrqapgkerefvasinLDNDAtnyadsvkgrftisrddarntvylqmnslkpedtavyycaa
TAISPISIVDeywgqgtqvtvss#ATACCATGAAAGTTCAACTGGTTGAATCTGGTGGCGGTTTAGTTCAAGCCGGTGATAGCCTTCGTCTGAGCTGCGCCGCAAG
CGGAGCCATTGTGCATGTGTTCGCTATGGGTTGGTTTCGCCAGGCACCTGGTAAAGAACGTGAATTTGTGGCATCCATTAACCTTGACAACGACGCTACCAA
CTATGCCGATTCTGTTAAAGGTCGCTTCACTATTTCACGCGATGATGCGCGTAATACCGTGTACTTACAGATGAACTCACTGAAACCTGAGGATACCGCCGTT
TATTATTGTGCGGCAACGGCTATTAGCCCTATCTCTATTGTGGATGAGTATTGGGGTCAGGGCACACAAGTCACGGTCTCAAGCGGCGGAAGCTCTGGTGA
ACAGAAACTGATTTCAGAAGCCAA
AIVHVFA#LDNDA#TIFMDSDLG#egqlvesggglvqagdslrlscaasgAIVHVFAmgwfrqapgkerefvasinLDNDAtnyadsvkgrftisrddarntvylqmnslkpedtavyycn
aTIFMDSDLGdywgqgtqgtvss#ACCATGGAAGGTCAACTGGTTGAATCTGGTGGCGGTTTAGTTCAAGCCGGTGATAGCCTTCGTCTGAGCTGCGCCGCAAG
CGGAGCCATTGTGCATGTGTTCGCTATGGGTTGGTTTCGCCAGGCACCTGGTAAAGAACGTGAATTTGTGGCATCCATTAACCTTGACAACGACGCTACCAA
CTATGCCGATTCTGTTAAAGGTCGCTTCACTATTTCACGCGATGATGCGCGTAATACCGTGTACTTACAGATGAACTCACTGAAACCTGAGGATACCGCCGTT
TATTATTGTAATGCAACCATCTTTATGGATTCCGATTTGGGTGACTATTGGGGTCAGGGCACACAAGGCACGGTCTCAAGCGGCGGAAGCTCTGGTGAAAA
GAAAATGATTTCAGAAGCCAA
AIVHVFA#LDNDA#TIFMDSDLG#evqlvesggglvqagdslrlscdasgAIVHVFAmgwfrqapgkerefvasinLDNDAtnyadsvkgrftisrddarntvylqmnslkpedtavyycn
aTIFMDSDLGdywgqgtqgtvss#AGATATACCATGGAAGTTCAACTGGTTGAATCTGGTGGCGGTTTAGTTCAAGCCGGTGATAGCCTTCGTCTGAGCTGCGA
CGCAAGCGGAGCCATTGTGCATGTGTTCGCTATGGGTTGGTTTCGCCAGGCACCTGGTAAAGAACGTGAATTTGTGGCATCCATTAACCTTGACAACGACGC
TACCAACTATGCCGATTCTGTTAAAGGTCGCTTCACTATTTCACGCGATGATGCGCGTAATACCGTGTACTTACAGATGAACTCACTGAAACCTGAGGATACC
GCCGTTTATTATTGTAATGCAACCATCTTTATGGATTCCGATTTGGGTGACTATTGGGGTCAGGGCACACAAGGCACGGTCTCAAGCGGCGGAAGCTCTGGT
GAACAGAAAATGATTTCAGAAGCCAA

TABLE 7
List of RBD binder clusters. A list containing key information for all predicted RBD binding clusters. Cluster ID, size, CDR representative sequences, CDR consensus sequences, CDR scores
(see Materials and Methods), and whether each CDR is unique to RBD and not found in EGFP clusters were shown.
Column 3 - CDR1_rep - SEQ ID NOS: 2756-3644
Column 4 - CDR2_rep: SEQ ID NOS: 3645-4551
Column 5 - CDR3_rep: SEQ ID NOS: 4552-5460

| clus- | clus- | | | | | | | | | | | | | |
|---|---|---|---|---|---|---|---|---|---|---|---|---|---|---|
| ter_{—} | ter_{—} | | | | | | | CDR1_{—} | CDR2_{—} | CDR3_{—} | total_{—} | CDR1_{—} | CDR2_{—} | CDR3_{—} |
| ID | size | CDR1_rep | CDR2_rep | CDR3_rep | CDR1_consensus | CDR2_consensus | CDR3_consensus | score | score | score | score | unique | unique | unique |
| 17 | 991 | YSCDTVR | IVSGW | QLVANTRLPVAVM | Y99B00.S99B00. | I99B00.V99B00. | B00 | 21 | 15 | 0 | 36 | Yes | Yes | Yes |
| | | | | | C99C99.D99B00. | S99B00.G99B00. | | | | | | | | |
| | | | | | T99B00.V99B00. | W99W99. | | | | | | | | |
| | | | | | R99R99. | | | | | | | | | |
| 22 | 679 | DCHVLWR | LDGLF | MPFF | D99D99.C99B00. | L99B00.D99B00. | B00 | 21 | 15 | 0 | 36 | Yes | Yes | Yes |
| | | | | | H99B00.V99B00. | G99B00.L99B00. | | | | | | | | |
| | | | | | L99L99.W99B00. | F99B00. | | | | | | | | |
| | | | | | R99B00. | | | | | | | | | |
| 112 | 574 | AIVHVFA | LDNDA | TIFMDSDLG | A99B00.I99I99. | L99L99.D99D99. | T67B00.I59B00. | 21 | 15 | 19 | 55 | Yes | Yes | Yes |
| | | | | | V99B00.H99B00. | N99B00.D99D99. | F60B00.M58B00. | | | | | | | |
| | | | | | V99V99.F99B00. | A99A99. | D59B00.S82B00. | | | | | | | |
| | | | | | A99B00. | | D58B00.L58B00. | | | | | | | |
| | | | | | | | G59B00. | | | | | | | |
| 67 | 393 | DYCMDCF | PYSFK | NPLDPNWTNS | D99D99.Y99B00. | P99B00.Y99B00. | B00B00.P35B00. | 21 | 15 | 11 | 47 | Yes | Yes | Yes |
| | | | | | C99C99.M99M99. | S99B00.F99B00. | L47B00.I40B00. | | | | | | | |
| | | | | | D99D99.C99B00. | K99B00. | B00B00.B00B00. | | | | | | | |
| | | | | | F99F99. | | B00B00.T51B00. | | | | | | | |
| | | | | | | | B00B00.S39B00. | | | | | | | |
| 33 | 31 | PYCMGNQ | QMSRQ | LRSFLE | P99B00.Y97B00. | Q99B00.M99B00. | L99B00.R99B00. | 21 | 15 | 18 | 54 | Yes | Yes | Yes |
| | | | | | C99B00.M99B00. | S99B00.R98B00. | S99B00.F99B00. | | | | | | | |
| | | | | | G99B00.N98B00. | Q99B00. | L99B00.E99B00. | | | | | | | |
| | | | | | Q99B00. | | | | | | | | | |
| 129 | 225 | WFQRQVY | RTLDS | ICYNRRDVML | W99W99.F99F99. | R99B00.T99T99. | I99B00.C99B00. | 21 | 15 | 30 | 66 | Yes | Yes | Yes |
| | | | | | Q98B00.R99R99. | L99B00.D99D99. | Y99Y99.N99B00. | | | | | | | |
| | | | | | Q99B00.V99B00. | S99S99. | R99B00.R99B00. | | | | | | | |
| | | | | | Y99Y99. | | D99B00.V98B00. | | | | | | | |
| | | | | | | | M99B00.L99B00. | | | | | | | |
| 278 | 202 | FQLSCYC | QHAFR | AETPNILVYE | F99F99.Q99B00. | Q99Q99.H99H99. | B00 | 21 | 15 | 0 | 36 | Yes | Yes | Yes |
| | | | | | L99L99.S99S99. | A99B00.F99F99. | | | | | | | | |
| | | | | | C99C99.Y99B00. | R99R99. | | | | | | | | |
| | | | | | C99C99. | | | | | | | | | |
| 75 | 198 | VCGIVSR | FMNFI | FTRFTH | V99V99.C99C99. | F99F99.M99B00. | F99B00.T99B00. | 21 | 15 | 18 | 54 | Yes | Yes | Yes |
| | | | | | G99G99.I99I99. | N99N99.F99B00. | R99B00.F99B00. | | | | | | | |
| | | | | | V99V99.S99S99. | I99I99. | T99T99.H99B00. | | | | | | | |
| | | | | | R98B00. | | | | | | | | | |
| 90 | 185 | IGLTWVC | TWNAS | FTSAVH | I96B00.G99B00. | T99B00.W99B00. | F99F99.T99T99. | 21 | 15 | 18 | 54 | Yes | Yes | Yes |
| | | | | | L99B00.T99T99. | N99N99.A99A99. | S99S99.A99B00. | | | | | | | |
| | | | | | W99B00.V98B00. | S99S99. | V99V99.H99B00. | | | | | | | |
| | | | | | C99B00. | | | | | | | | | |
| 7 | 172 | KTGRSVI | RGDDT | VYFVNDCIV | K98B00.T98B00. | R99B00.G99B00. | V83B00.Y83B00. | 21 | 15 | 27 | 63 | Yes | Yes | Yes |
| | | | | | G99B00.R99B00. | D99B00.D98B00. | F83B00.V83B00. | | | | | | | |
| | | | | | S98B00.V98B00. | T99B00. | N83B00.D83B00. | | | | | | | |
| | | | | | I97B00. | | C83B00.I83B00. | | | | | | | |
| | | | | | | | V83B00. | | | | | | | |
| 227 | 164 | AFGSNVF | PHHTV | AIYRNASVL | A99B00.F99F99. | P99P99.H99H99. | A99B00.I99I99. | 21 | 15 | 27 | 63 | Yes | Yes | Yes |
| | | | | | G99G99.S99B00. | H99H99.T99T99. | Y99Y99.R99B00. | | | | | | | |
| | | | | | N98B00.V99V99. | V99B00. | N99B00.A99A99. | | | | | | | |
| | | | | | F99B00. | | S99S99.V99B00. | | | | | | | |
| | | | | | | | L99B00. | | | | | | | |
| 82 | 12 | IRHDFLL | FGGRR | SLFGEDLSVR | I98B00.R98B00. | F99F99.G99G99. | S52B00.L53B00. | 21 | 15 | 20 | 56 | Yes | Yes | Yes |
| | | | | | H99H99.D98B00. | G99G99.R99R99. | F52B00.G52B00. | | | | | | | |
| | | | | | F99F99.L99B00. | R99R99. | E52B00.D61B00. | | | | | | | |
| | | | | | L99L99. | | L79B00.S52B00. | | | | | | | |
| | | | | | | | V52B00.R80B00. | | | | | | | |
| 163 | 156 | IGERCLF | AFSFR | LDTAALFPEF | I98B00.G99B00. | A99A99.F99F99. | L98B00.D86B00. | 21 | 15 | 30 | 66 | Yes | Yes | Yes |
| | | | | | E99E99.R99B00. | S99S99.F99F99. | T99B00.A87B00. | | | | | | | |
| | | | | | C99C99.L99L99. | R99R99. | A86B00.L86B00. | | | | | | | |
| | | | | | F99F99. | | F86B00.P99B00. | | | | | | | |
| | | | | | | | E99B00.F86B00. | | | | | | | |
| 120 | 15 | IFSGCVF | RSSQG | MCLDGCYIC | I97B00.F99B00. | R99B00.S99S99. | M99B00.C98B00. | 21 | 15 | 27 | 63 | Yes | Yes | Yes |
| | | | | | S99S99.G99G99. | S99S99.Q99B00. | L99B00.D99B00. | | | | | | | |
| | | | | | C99C99.V99B00. | G99G99. | G99B00.C98B00. | | | | | | | |
| | | | | | F99F99. | | Y98B00.I99B00. | | | | | | | |
| | | | | | | | C99B00. | | | | | | | |
| 46 | 132 | CNARQVY | RGPED | AYLGEDMSCS | C98B00.N96B00. | R99R99.G99B00. | A99A99.Y99B00. | 21 | 15 | 30 | 66 | Yes | Yes | Yes |
| | | | | | A99B00.R99B00. | P99B00.E98B00. | L99L99.G99G99. | | | | | | | |
| | | | | | Q99B00.V99B00. | D98B00. | E99E99.D99D99. | | | | | | | |
| | | | | | Y98B00. | | M99B00.S99S99. | | | | | | | |
| | | | | | | | C99C99.S99S99. | | | | | | | |
| 210 | 124 | FKLPCL | DHGGR | VFIVKYEEELLVE | F98B00.K95B00. | D99D99.H99B00. | V99V99.F99F99. | 18 | 15 | 39 | 72 | Yes | Yes | Yes |
| | | | | | L99L99.P99P99. | G99G99.G99G99. | I99I99.V99V99. | | | | | | | |
| | | | | | C99C99.L99L99. | R99B00. | K99K99.Y99Y99. | | | | | | | |
| | | | | | | | E99E99.E99E99. | | | | | | | |
| | | | | | | | E99E99.L99B00. | | | | | | | |
| | | | | | | | L99L99.V99V99. | | | | | | | |
| | | | | | | | E99E99. | | | | | | | |
| 137 | 118 | IWMGTRV | GCYGT | LTRPPFDPLW | I96B00.W99B00. | G99G99.C99B00. | L99L99.T99T99. | 21 | 15 | 30 | 66 | Yes | Yes | Yes |
| | | | | | M98B00.G99B00. | Y98B00.G99G99. | R99R99.P99P99. | | | | | | | |
| | | | | | T98B00.R99B00. | T99T99. | P99P99.F99F99. | | | | | | | |
| | | | | | V98B00. | | D99D99.P99P99. | | | | | | | |
| | | | | | | | L99L99.W99W99. | | | | | | | |
| 29 | 116 | IMIGTQS | ANEMF | ITAMGW | I97B00.M99M99. | A99A99.N99N99. | B00 | 21 | 15 | 0 | 36 | Yes | Yes | Yes |
| | | | | | I98B00.G99G99. | E99E99.M99M99. | | | | | | | | |
| | | | | | T99T99.Q99B00. | F99F99. | | | | | | | | |
| | | | | | S99S99. | | | | | | | | | |
| 54 | 11 | ALCRRVY | CAGWR | AVFASDGNVV | A99A99.L99L99. | C99C99.A99A99. | A99A99.V99V99. | 21 | 15 | 30 | 66 | Yes | Yes | Yes |
| | | | | | C99C99.R99R99. | G99G99.W99W99. | F99F99.A99A99. | | | | | | | |
| | | | | | R99R99.V99B00. | R99R99. | S99B00.D99D99. | | | | | | | |
| | | | | | Y98B00. | | G99B00.N99N99. | | | | | | | |
| | | | | | | | V99V99.V99V99. | | | | | | | |
| 71 | 108 | LCLSVLA | GSAAA | WLLTYSHDVM | L99L99.C99B00. | G99G99.S99S99. | W99W99.L99L99. | 21 | 15 | 30 | 66 | Yes | Yes | Yes |
| | | | | | L99L99.S99S99. | A99A99.A99A99. | L99L99.T99T99. | | | | | | | |
| | | | | | V99V99.L99L99. | A99A99. | Y99B00.S99S99. | | | | | | | |
| | | | | | A99A99. | | H99H99.D99D99. | | | | | | | |
| | | | | | | | V99V99.M99M99. | | | | | | | |
| 189 | 100 | TACGLLG | SVTSF | VCSCMY | T99T99.A99A99. | S99S99.V99V99. | V99B00.C99B00. | 21 | 15 | 18 | 54 | Yes | Yes | Yes |
| | | | | | C99C99.G99G99. | T99T99.S99S99. | S99S99.C99B00. | | | | | | | |
| | | | | | L99L99.L99L99. | F99B00. | M99B00.Y99B00. | | | | | | | |
| | | | | | G99G99. | | | | | | | | | |
| 62 | 99 | VLGVTIS | LSFDV | RTRFGH | V99V99.L99L99. | L98B00.S99S99. | R99R99.T99T99. | 21 | 15 | 18 | 54 | Yes | Yes | Yes |
| | | | | | G98B00.V98B00. | F99F99.D99D99. | R99R99.F99F99. | | | | | | | |
| | | | | | T97B00.I98B00. | V99V99. | G99G99.H98B00. | | | | | | | |
| | | | | | S98B00. | | | | | | | | | |
| 209 | 92 | ASFYNTM | SVSHV | KVVGVA | A99A99.S99S99. | S99S99.V99V99. | K98B00.V98B00. | 21 | 15 | 18 | 54 | Yes | Yes | Yes |
| | | | | | F99F99.Y99Y99. | S99S99.H99H99. | V98B00.G98B00. | | | | | | | |
| | | | | | N99N99.T99T99. | V99V99. | V98B00.A98B00. | | | | | | | |
| | | | | | M97B00. | | | | | | | | | |
| 213 | 90 | ATNFCVQ | TFDDY | SLSPDCLHIS | A99A99.T99T99. | T98B00.F99F99. | S92B00.L92B00. | 21 | 15 | 30 | 66 | Yes | Yes | Yes |
| | | | | | N94B00.F99F99. | D99D99.D98B00. | S92B00.P91B00. | | | | | | | |
| | | | | | C99C99.V99V99. | Y99Y99. | D93B00.C91B00. | | | | | | | |
| | | | | | Q99Q99. | | L92B00.H91B00. | | | | | | | |
| | | | | | | | I91B00.S93B00. | | | | | | | |
| 134 | 87 | FWSNAIF | SSA | IDCYGV | F99F99.W99W99. | S99S99.S99S99. | I99I99.D99D99. | 21 | 9 | 18 | 48 | Yes | Yes | Yes |
| | | | | | S99S99.N99N99. | A99A99. | C98B00.Y99Y99. | | | | | | | |
| | | | | | A99A99.I99I99. | | G98B00.V99V99. | | | | | | | |
| | | | | | F99F99. | | | | | | | | | |
| 24 | 87 | RLLRLVY | TCVGA | ILFDTNVN | R99R99.L99L99. | T99T99.C99C99. | I99I99.L99L99. | 21 | 15 | 24 | 60 | Yes | Yes | Yes |
| | | | | | L99L99.R99R99. | V99V99.G99G99. | F99F99.D99D99. | | | | | | | |
| | | | | | L99L99.V99V99. | A99A99. | T99T99.N98B00. | | | | | | | |
| | | | | | Y99Y99. | | V99V99.N99N99. | | | | | | | |
| 65 | 83 | LRSVVF | TIALD | VIYNGCYTM | L99L99.R97B00. | T98B00.I98B00. | V93B00.I90B00. | 18 | 15 | 27 | 60 | Yes | Yes | Yes |
| | | | | | S97B00.V98B00. | A98B00.L97B00. | Y90B00.N90B00. | | | | | | | |
| | | | | | V98B00.F98B00. | D98B00. | G90B00.C90B00. | | | | | | | |
| | | | | | | | Y90B00.T95B00. | | | | | | | |
| | | | | | | | M90B00. | | | | | | | |
| 180 | 82 | MKCIHAG | LDSSW | FVGCHYSNYYDFA | M98B00.K96B00. | L98B00.D99D99. | F98B00.V98B00. | 21 | 15 | 39 | 75 | Yes | Yes | Yes |
| | | | | | C99C99.I99I99. | S99S99.S99S99. | G98B00.C98B00. | | | | | | | |
| | | | | | H99H99.A99A99. | W99W99. | H98B00.Y98B00. | | | | | | | |
| | | | | | G99G99. | | S98B00.N98B00. | | | | | | | |
| | | | | | | | Y98B00.Y98B00. | | | | | | | |
| | | | | | | | D98B00.F97B00. | | | | | | | |
| | | | | | | | A98B00. | | | | | | | |
| 378 | 82 | WVTFMVF | QVTGH | CQNVAKSAWYAVG | W99W99.V99V99. | Q99Q99.V99V99. | B00 | 21 | 15 | 0 | 36 | Yes | Yes | Yes |
| | | | | | T97B00.F99F99. | T99T99.G99G99. | | | | | | | | |
| | | | | | M99M99.V99V99. | H99H99. | | | | | | | | |
| | | | | | F99F99. | | | | | | | | | |
| 240 | 78 | RSVG | RCVSD | WLLTPGCIR | R99R99.S99S99. | R99R99.C99C99. | W70B00.L70B00. | 12 | 15 | 18 | 45 | Yes | Yes | Yes |
| | | | | | V99V99.G99G99. | V99V99.S99S99. | L70B00.T70B00. | | | | | | | |
| | | | | | | D99D99. | P69B00.G70B00. | | | | | | | |
| | | | | | | | C70B00.I70B00. | | | | | | | |
| | | | | | | | R70B00. | | | | | | | |
| 214 | 77 | RARNIVY | LGEGL | HRFDTTLMCVVRA | R99R99.A98B00. | L99L99.G99G99. | H99H99.R99R99. | 21 | 15 | 39 | 75 | Yes | Yes | Yes |
| | | | | | R98B00.N98B00. | E99E99.G99G99. | F99F99.D99D99. | | | | | | | |
| | | | | | I99I99.V99V99. | L99L99. | T99T99.T99T99. | | | | | | | |
| | | | | | Y99Y99. | | L99L99.M99M99. | | | | | | | |
| | | | | | | | C99C99.V99V99. | | | | | | | |
| | | | | | | | V99V99.R99R99. | | | | | | | |
| | | | | | | | A99A99. | | | | | | | |
| 397 | 77 | FPCSYVF | CVTGI | TNNTLR | F98B00.P98B00. | C99C99.V99V99. | T99T99.N99N99. | 21 | 15 | 18 | 54 | Yes | Yes | Yes |
| | | | | | C98B00.S98B00. | T99T99.G99G99. | N99N99.T99T99. | | | | | | | |
| | | | | | Y98B00.V98B00. | I99I99. | L99L99.R99R99. | | | | | | | |
| | | | | | F98B00. | | | | | | | | | |
| 133 | 75 | SRCALVV | SVTCS | INNELN | S99S99.R99R99. | S99S99.V99V99. | I97B00.N97B00. | 21 | 15 | 18 | 54 | Yes | Yes | Yes |
| | | | | | C99C99.A99A99. | T99T99.C99C99. | N97B00.E97B00. | | | | | | | |
| | | | | | L99L99.V99V99. | S99S99. | L97B00.N97B00. | | | | | | | |
| | | | | | V99V99. | | | | | | | | | |
| 39 | 75 | YVMMWCE | PRNFD | LVCPGS | Y99Y99.V99V99. | P99P99.R98B00. | L99L99.V99V99. | 21 | 15 | 18 | 54 | Yes | Yes | Yes |
| | | | | | M98B00.M99M99. | N99N99.F99F99. | C99C99.P99P99. | | | | | | | |
| | | | | | W99W99.C99C99. | D99D99. | G99G99.S99S99. | | | | | | | |
| | | | | | E98B00. | | | | | | | | | |
| 501 | 73 | VYVGSAT | AYLGY | QCSVIL | V99V99.Y95B00. | A99A99.Y99Y99. | Q98B00.C98B00. | 21 | 15 | 18 | 54 | Yes | Yes | Yes |
| | | | | | V97B00.G99G99. | L99L99.G99G99. | S99S99.V99V99. | | | | | | | |
| | | | | | S99S99.A99A99. | Y99Y99. | I99I99.L99L99. | | | | | | | |
| | | | | | T98B00. | | | | | | | | | |
| 78 | 71 | IANKIVF | QSTGV | VRFRTHGAVT | I98B00.A99A99. | Q99Q99.S98B00. | V98B00.R98B00. | 21 | 15 | 30 | 66 | Yes | Yes | Yes |
| | | | | | N97B00.K99K99. | T99T99.G99G99. | F98B00.R98B00. | | | | | | | |
| | | | | | I99I99.V99V99. | V99V99. | T98B00.H98B00. | | | | | | | |
| | | | | | F99F99. | | G98B00.A98B00. | | | | | | | |
| | | | | | | | V98B00.T98B00. | | | | | | | |
| 116 | 70 | FLFRVVH | ISSGG | HFFTADLSVV | F99F99.L99L99. | I99I99.S99S99. | H99H99.F99F99. | 21 | 15 | 30 | 66 | Yes | Yes | Yes |
| | | | | | F99F99.R99R99. | S99S99.G99G99. | F98B00.T99T99. | | | | | | | |
| | | | | | V99V99.V99V99. | G99G99. | A99A99.D99D99. | | | | | | | |
| | | | | | H99H99. | | L99L99.S99S99. | | | | | | | |
| | | | | | | | V99V99.V99V99. | | | | | | | |
| 95 | 68 | SVYGPSN | IAAQV | RTVTPNYMID | S95B00.V99V99. | I99I99.A99A99. | R97B00.T97B00. | 21 | 15 | 30 | 66 | Yes | Yes | Yes |
| | | | | | Y99Y99.G99G99. | A99A99.Q99Q99. | V97B00.T97B00. | | | | | | | |
| | | | | | P98B00.S99S99. | V99V99. | P97B00.N97B00. | | | | | | | |
| | | | | | N98B00. | | Y97B00.M97B00. | | | | | | | |
| | | | | | | | I97B00.D97B00. | | | | | | | |
| 228 | 65 | HFVRIMA | NRHSC | YLLDAHSTCN | H98B00.F98B00. | N99N99.R99R99. | Y99Y99.L99L99. | 21 | 15 | 30 | 66 | Yes | Yes | Yes |
| | | | | | V99V99.R98B00. | H99H99.S99S99. | L99L99.D99D99. | | | | | | | |
| | | | | | I98B00.M98B00. | C98B00. | A99A99.H99H99. | | | | | | | |
| | | | | | A96B00. | | S99S99.T99T99. | | | | | | | |
| | | | | | | | C99C99.N99N99. | | | | | | | |
| 148 | 64 | GLYVSIV | NTRQW | NPHLHPHLS | G98B00.L99L99. | N99N99.T99T99. | N98B00.P99P99. | 21 | 15 | 27 | 63 | Yes | Yes | Yes |
| | | | | | Y99Y99.V99V99. | R99R99.Q99Q99. | H98B00.L99L99. | | | | | | | |
| | | | | | S99S99.I98B00. | W99W99. | H98B00.P98B00. | | | | | | | |
| | | | | | V99V99. | | H98B00.L98B00. | | | | | | | |
| | | | | | | | S98B00. | | | | | | | |
| 60 | 64 | LVLSYFT | AQDRG | LYNLFE | L99L99.V99V99. | A99A99.Q99Q99. | L96B00.Y98B00. | 21 | 15 | 18 | 54 | Yes | Yes | Yes |
| | | | | | L99L99.S99S99. | D99D99.R99R99. | N98B00.L98B00. | | | | | | | |
| | | | | | Y99Y99.F99F99. | G99G99. | F98B00.E98B00. | | | | | | | |
| | | | | | T99T99. | | | | | | | | | |
| 315 | 63 | GLYWQND | RYPCY | IPCRWFWIE | G99G99.L99L99. | R99R99.Y98B00. | I99I99.P99P99. | 21 | 15 | 27 | 63 | Yes | Yes | Yes |
| | | | | | Y99Y99.W99W99. | P99P99.C99C99. | C99C99.R99R99. | | | | | | | |
| | | | | | Q99Q99.N99N99. | Y99Y99. | W99W99.F99F99. | | | | | | | |
| | | | | | D99D99. | | W99W99.I99I99. | | | | | | | |
| | | | | | | | E99E99. | | | | | | | |
| 14 | 62 | LLAVPVD | WCGLS | IRRASFTFYW | L98B00.L96B00. | W98B00.C98B00. | I99I99.R99R99. | 21 | 15 | 30 | 66 | Yes | Yes | Yes |
| | | | | | A98B00.V98B00. | G98B00.L98B00. | R99R99.A99A99. | | | | | | | |
| | | | | | P96B00.V99V99. | S98B00. | S99S99.F99F99. | | | | | | | |
| | | | | | D96B00. | | T99T99.F95B00. | | | | | | | |
| | | | | | | | Y95B00.W95B00. | | | | | | | |
| 259 | 62 | ACCDAH | ATPSA | IQWHARGRY | A99A99.C99C99. | A99A99.T99T99. | I98B00.Q98B00. | 18 | 15 | 27 | 60 | Yes | Yes | Yes |
| | | | | | C98B00.D96B00. | P99P99.S99S99. | W98B00.H98B00. | | | | | | | |
| | | | | | A99A99.H98B00. | A99A99. | A98B00.R98B00. | | | | | | | |
| | | | | | | | G98B00.R98B00. | | | | | | | |
| | | | | | | | Y98B00. | | | | | | | |
| 290 | 62 | TGFCHYW | ASSSR | ADMYFF | T98B00.G98B00. | A99A99.S99S99. | A99A99.D98B00. | 21 | 15 | 18 | 54 | Yes | Yes | Yes |
| | | | | | F96B00.C98B00. | S99S99.S99S99. | M99M99.Y99Y99. | | | | | | | |
| | | | | | H98B00.Y98B00. | R99R99. | F99F99.F99F99. | | | | | | | |
| | | | | | W98B00. | | | | | | | | | |
| 474 | 62 | HSAHASQ | RSAAN | PWALDPAFRA | H98B00.S98B00. | R99R99.S99S99. | P99P99.W99W99. | 21 | 15 | 30 | 66 | Yes | Yes | Yes |
| | | | | | A98B00.H98B00. | A98B00.A99A99. | A99A99.L99L99. | | | | | | | |
| | | | | | A98B00.S98B00. | N99N99. | D99D99.P99P99. | | | | | | | |
| | | | | | Q96B00. | | A99A99.F99F99. | | | | | | | |
| | | | | | | | R98B00.A98B00. | | | | | | | |
| 485 | 60 | VLLRRVF | CNNHS | HTLSNKFFVN | V99V99.L99L99. | C99C99.N98B00. | H96B00.T98B00. | 21 | 15 | 23 | 59 | Yes | Yes | Yes |
| | | | | | L99L99.R99R99. | N99N99.H98B00. | L98B00.S98B00. | | | | | | | |
| | | | | | R99R99.V99V99. | S99S99. | N51D46.K51N46. | | | | | | | |
| | | | | | F99F99. | | F51Y46.F50S46. | | | | | | | |
| | | | | | | | V51I46.N51I46. | | | | | | | |
| 52 | 60 | VTRGLLN | ASMLT | NIFSDTESVR | V99V99.T99T99. | A99A99.S99S99. | N99N99.I99I99. | 21 | 15 | 30 | 66 | Yes | Yes | Yes |
| | | | | | R99R99.G99G99. | M99M99.L98B00. | F99F99.S99S99. | | | | | | | |
| | | | | | L99L99.L99L99. | T99T99. | D99D99.T99T99. | | | | | | | |
| | | | | | N98B00. | | E99E99.S99S99. | | | | | | | |
| | | | | | | | V99V99.R99R99. | | | | | | | |
| 147 | 57 | GVAQVVF | VDAMN | FRLQSPGLVT | G98B00.V98B00. | V99V99.D99D99. | F99F99.R99R99. | 21 | 15 | 30 | 66 | Yes | Yes | Yes |
| | | | | | A98B00.Q98B00. | A99A99.M99M99. | L99L99.Q99Q99. | | | | | | | |
| | | | | | V99V99.V98B00. | N99N99. | S99S99.P99P99. | | | | | | | |
| | | | | | F98B00. | | G98B00.L99L99. | | | | | | | |
| | | | | | | | V99V99.T99T99. | | | | | | | |
| 30 | 57 | TFCMFVC | SYSEG | WISCTQ | T98B00.F99F99. | S99S99.Y99Y99. | W99W99.I99I99. | 21 | 15 | 18 | 54 | Yes | Yes | Yes |
| | | | | | C99C99.M99M99. | S99S99.E99E99. | S99S99.C99C99. | | | | | | | |
| | | | | | F99F99.V99V99. | G99G99. | T98B00.Q98B00. | | | | | | | |
| | | | | | C99C99. | | | | | | | | | |
| 170 | 56 | LRCDSAS | LGG | HTINRYAPWLGCA | L99L99.R99R99. | L99L99.G99G99. | H99H99.T99T99. | 21 | 9 | 39 | 69 | Yes | Yes | Yes |
| | | | | | C99C99.D99D99. | G99G99. | I99I99.N99N99. | | | | | | | |
| | | | | | S99S99.A99A99. | | R99R99.Y99Y99. | | | | | | | |
| | | | | | S99S99. | | A99A99.P99P99. | | | | | | | |
| | | | | | | | W99W99.L99L99. | | | | | | | |
| | | | | | | | G99G99.C99C99. | | | | | | | |
| | | | | | | | A99A99. | | | | | | | |
| 273 | 56 | TLRSCVN | SGSVH | MCLTLTWSIA | T98B00.L98B00. | S98B00.G98B00. | M57H42.C57R42. | 21 | 15 | 20 | 56 | Yes | Yes | Yes |
| | | | | | R98B00.S98B00. | S98B00.V98B00. | L57F42.T57V42. | | | | | | | |
| | | | | | C98B00.V98B00. | H98B00. | L57C42.T57G42. | | | | | | | |
| | | | | | N98B00. | | W57S42.S57V42. | | | | | | | |
| | | | | | | | I57R42.A57B42. | | | | | | | |
| 286 | 56 | R | PHNLG | MNHAYPIFGLADD | R99R99. | P99P99.H99H99. | M99M99.N99N99. | 3 | 15 | 39 | 57 | Yes | Yes | Yes |
| | | | | | | N99N99.L99L99. | H99H99.A99A99. | | | | | | | |
| | | | | | | G98B00. | Y99Y99.P99P99. | | | | | | | |
| | | | | | | | I99I99.F99F99. | | | | | | | |
| | | | | | | | G99G99.L99L99. | | | | | | | |
| | | | | | | | A99A99.D99D99. | | | | | | | |
| | | | | | | | D99D99. | | | | | | | |
| 19 | 55 | VIFRESF | PGAYR | FDPTSGTLAV | V99V99.I99I99. | P99P99.G98B00. | B00 | 21 | 15 | 0 | 36 | Yes | Yes | Yes |
| | | | | | F99F99.R99R99. | A99A99.Y99Y99. | | | | | | | | |
| | | | | | E99E99.S99S99. | R98B00. | | | | | | | | |
| | | | | | F99F99. | | | | | | | | | |
| 42 | 55 | RCGSVVF | ISGEF | CYYVNHGCCV | R99R99.C99C99. | I99I99.S99S99. | C99C99.Y99Y99. | 21 | 15 | 30 | 66 | Yes | Yes | Yes |
| | | | | | G99G99.S98B00. | G99G99.E98B00. | Y99Y99.V99V99. | | | | | | | |
| | | | | | V99V99.V99V99. | F99F99. | N99N99.H99H99. | | | | | | | |
| | | | | | F99F99. | | G99G99.C99C99. | | | | | | | |
| | | | | | | | C99C99.V99V99. | | | | | | | |
| 165 | 54 | GILLRTC | FRLAL | TPWPAFIGLV | G99G99.I99I99. | F99F99.R99R99. | T99T99.P99P99. | 21 | 15 | 30 | 66 | Yes | Yes | Yes |
| | | | | | L99L99.L99L99. | L99L99.A99A99. | W98B00.P99P99. | | | | | | | |
| | | | | | R99R99.T99T99. | L99L99. | A99A99.F99F99. | | | | | | | |
| | | | | | C99C99. | | I99I99.G99G99. | | | | | | | |
| | | | | | | | L99L99.V99V99. | | | | | | | |
| 87 | 54 | SLRLVVY | RCGGL | SNLTSNSTGNEVT | S94B00.L99L99. | R99R99.C99C99. | B00 | 21 | 15 | 0 | 36 | Yes | Yes | Yes |
| | | | | | R99R99.L99L99. | G99G99.G98B00. | | | | | | | | |
| | | | | | V99V99.V99V99. | L99L99. | | | | | | | | |
| | | | | | Y99Y99. | | | | | | | | | |
| 211 | 54 | FVIPRYA | YMLRC | IDPTPAVVNL | F99F99.V98B00. | Y98B00.M99M99. | I99I99.D98B00. | 21 | 15 | 30 | 66 | Yes | Yes | Yes |
| | | | | | I96B00.P99P99. | L99L99.R98B00. | P99P99.T99T99. | | | | | | | |
| | | | | | R98B00.Y98B00. | C99C99. | P99P99.A99A99. | | | | | | | |
| | | | | | A99A99. | | V98B00.V99V99. | | | | | | | |
| | | | | | | | N99N99.L99L99. | | | | | | | |
| 266 | 54 | PVFRCVY | NDIND | ISLQTNGSLQ | P99P99.V99V99. | N99N99.D99D99. | I51A48.S51A46. | 21 | 15 | 22 | 58 | Yes | Yes | Yes |
| | | | | | F99F99.R99R99. | I99I99.N99N99. | L51Y46.Q51L46. | | | | | | | |
| | | | | | C99C99.V99V99. | D99D99. | T98B00.N51P46. | | | | | | | |
| | | | | | Y99Y99. | | G53F46.S98B00. | | | | | | | |
| | | | | | | | L51V48.Q51C46. | | | | | | | |
| 388 | 54 | VCRGVVA | DCHSD | FCLDTRSAATVC | V99V99.C99C99. | D99D99.C99C99. | B00 | 21 | 15 | 0 | 36 | Yes | Yes | Yes |
| | | | | | R99R99.G99G99. | H98B00.S99S99. | | | | | | | | |
| | | | | | V96B00.V98B00. | D99D99. | | | | | | | | |
| | | | | | A99A99. | | | | | | | | | |
| 256 | 53 | WGYRMRV | HPSCV | VYLVGNTMV | W99W99.G99G99. | H99H99.P99P99. | V99V99.Y99Y99. | 21 | 15 | 27 | 63 | Yes | Yes | Yes |
| | | | | | Y99Y99.R99R99. | S99S99.C99C99. | L99L99.V99V99. | | | | | | | |
| | | | | | M99M99.R99R99. | V99V99. | G99G99.N99N99. | | | | | | | |
| | | | | | V99V99. | | T99T99.M98B00. | | | | | | | |
| | | | | | | | V99V99. | | | | | | | |
| 91 | 53 | RLRHVVF | TTACG | ALYHPMHSVL | R99R99.L98B00. | T99T99.T99T99. | A94B00.L92B00. | 21 | 15 | 30 | 66 | Yes | Yes | Yes |
| | | | | | R98B00.H99H99. | A99A99.C99C99. | Y96B00.H94B00. | | | | | | | |
| | | | | | V99V99.V99V99. | G99G99. | P94B00.M94B00. | | | | | | | |
| | | | | | F99F99. | | H94B00.S94B00. | | | | | | | |
| | | | | | | | V94B00.L96B00. | | | | | | | |
| 93 | 52 | VLGILST | SFD | ATRWSS | V99V99.L99L99. | S98B00.F99F99. | A99A99.T99T99. | 21 | 0 | 18 | 48 | Yes | Yes | Yes |
| | | | | | G99G99.I99I99. | D99D99. | R99R99.W99W99. | | | | | | | |
| | | | | | L99L99.S99S99. | | S99S99.S99S99. | | | | | | | |
| | | | | | T99T99. | | | | | | | | | |
| 141 | 51 | DFANIHG | MYGEY | HIITSSFSGIESR | D98B00.F98B00. | M98B00.Y98B00. | H99H99.I99I99. | 21 | 15 | 39 | 75 | Yes | Yes | Yes |
| | | | | | A98B00.N98B00. | G98B00.E98B00. | I99I99.T99T99. | | | | | | | |
| | | | | | I98B00.H98B00. | Y98B00. | S99S99.S99S99. | | | | | | | |
| | | | | | G96B00. | | F98B00.S99S99. | | | | | | | |
| | | | | | | | G99G99.I99I99. | | | | | | | |
| | | | | | | | E99E99.S99S99. | | | | | | | |
| | | | | | | | R99R99. | | | | | | | |
| 292 | 50 | LDRAVL | DCGHQ | YRLTHDKVVS | L99L99.D98B00. | D99D99.C99C99. | Y99Y99.R99R99. | 18 | 15 | 30 | 63 | Yes | Yes | Yes |
| | | | | | R98B00.A98B00. | G99G99.H98B00. | L99L99.T99T99. | | | | | | | |
| | | | | | V99V99.L98B00. | Q99Q99. | H99H99.D98B00. | | | | | | | |
| | | | | | | | K99K99.V99V99. | | | | | | | |
| | | | | | | | V99V99.S99S99. | | | | | | | |
| 175 | 49 | ARDRLLG | ISFRL | WTEYSS | A99A99.R99R99. | I97B00.S99S99. | W99W99.T99T99. | 21 | 15 | 18 | 54 | Yes | Yes | Yes |
| | | | | | D99D99.R99R99. | F99F99.R99R99. | E99E99.Y99Y99. | | | | | | | |
| | | | | | L99L99.L99L99. | L99L99. | S99S99.S97B00. | | | | | | | |
| | | | | | G99G99. | | | | | | | | | |
| 202 | 49 | MPAFGGR | TCNCW | FNLPTFYSFA | M97B00.P97B00. | T99T99.C99C99. | F99F99.N99N99. | 21 | 15 | 30 | 66 | Yes | Yes | Yes |
| | | | | | A95B00.F97B00. | N99N99.C99C99. | L99L99.P99P99. | | | | | | | |
| | | | | | G99G99.G97B00. | W99W99. | T99T99.F99F99. | | | | | | | |
| | | | | | R97B00. | | Y99Y99.S99S99. | | | | | | | |
| | | | | | | | F99F99.A99A99. | | | | | | | |
| 277 | 49 | HSQHVFY | AYDAN | GIFEMFGCR | H99H99.S99S99. | A99A99.Y99Y99. | G99G99.I99I99. | 21 | 15 | 27 | 63 | Yes | Yes | Yes |
| | | | | | Q99Q99.H97B00. | D99D99.A99A99. | F99F99.E99E99. | | | | | | | |
| | | | | | V99V99.F99F99. | N99N99. | M97B00.F99F99. | | | | | | | |
| | | | | | Y99Y99. | | G99G99.C99C99. | | | | | | | |
| | | | | | | | R99R99. | | | | | | | |
| 56 | 49 | LGSGLLF | GCFNL | SALSSENDVR | L99L99.G99G99. | G99G99.C99C99. | S99S99.A99A99. | 21 | 15 | 30 | 66 | Yes | Yes | Yes |
| | | | | | S99S99.G99G99. | F99F99.N99N99. | L97B00.S99S99. | | | | | | | |
| | | | | | L99L99.L99L99. | L99L99. | S97B00.E99E99. | | | | | | | |
| | | | | | F99F99. | | N99N99.D99D99. | | | | | | | |
| | | | | | | | V99V99.R99R99. | | | | | | | |
| 118 | 48 | HTTRRIY | VDARC | VYFAGEGPV | H99H99.T99T99. | V99V99.D99D99. | V99V99.Y97B00. | 21 | 15 | 27 | 63 | Yes | Yes | Yes |
| | | | | | T97B00.R99R99. | A99A99.R99R99. | F99F99.A99A99. | | | | | | | |
| | | | | | R99R99.I99I99. | C99C99. | G99G99.E99E99. | | | | | | | |
| | | | | | Y99Y99. | | G99G99.P99P99. | | | | | | | |
| | | | | | | | V99V99. | | | | | | | |
| 293 | 48 | VFGIVRS | ITNSH | ISRFTT | V99V99.F99F99. | I99I99.T97B00. | I97B00.S99S99. | 21 | 15 | 18 | 54 | Yes | Yes | Yes |
| | | | | | G99G99.I99I99. | N99N99.S99S99. | R97B00.F97B00. | | | | | | | |
| | | | | | V99V99.R99R99. | H99H99. | T97B00.T97B00. | | | | | | | |
| | | | | | S99S99. | | | | | | | | | |
| 340 | 48 | HLDALVY | SNMHT | RTAFHDHWN | H99H99.L97B00. | S99S99.N99N99. | R99R99.T99T99. | 21 | 15 | 27 | 63 | Yes | Yes | Yes |
| | | | | | D99D99.A99A99. | M97B00.H99H99. | A99A99.F99F99. | | | | | | | |
| | | | | | L97B00.V99V99. | T99T99. | H99H99.D99D99. | | | | | | | |
| | | | | | Y99Y99. | | H99H99.W99W99. | | | | | | | |
| | | | | | | | N99N99. | | | | | | | |
| 371 | 48 | QAIVVSW | FGKHG | LIFDIHGYGF | Q99Q99.A99A99. | F99F99.G99G99. | L99L99.I99I99. | 21 | 15 | 30 | 66 | Yes | Yes | Yes |
| | | | | | I97B00.V99V99. | K99K99.H99H99. | F99F99.D99D99. | | | | | | | |
| | | | | | V99V99.S97B00. | G99G99. | I99I99.H99H99. | | | | | | | |
| | | | | | W99W99. | | G99G99.Y97B00. | | | | | | | |
| | | | | | | | G99G99.F99F99. | | | | | | | |
| 157 | 47 | SFRGLRW | VRLSC | HPIVCDLWV | S97B00.F99F99. | V99V99.R99R99. | H99H99.P99P99. | 21 | 15 | 27 | 63 | Yes | Yes | Yes |
| | | | | | R95B00.G99G99. | L99L99.S99S99. | I99I99.V99V99. | | | | | | | |
| | | | | | L99L99.R99R99. | C99C99. | C99C99.D99D99. | | | | | | | |
| | | | | | W99W99. | | L99L99.W99W99. | | | | | | | |
| | | | | | | | V99V99. | | | | | | | |
| 534 | 47 | RLVNVVY | AGNSW | CVFDTADVR | R99R99.L99L99. | A99A99.G99G99. | C91B00.V87B00. | 21 | 15 | 27 | 63 | Yes | Yes | Yes |
| | | | | | V99V99.N99N99. | N99N99.S99S99. | F91B00.D91B00. | | | | | | | |
| | | | | | V99V99.V99V99. | W99W99. | T93B00.A91B00. | | | | | | | |
| | | | | | Y97B00. | | D93B00.V95B00. | | | | | | | |
| | | | | | | | R91B00. | | | | | | | |
| 12 | 46 | DPRGLVY | CSTSC | AYCRQLLVT | D99D99.P99P99. | C99C99.S99S99. | A99A99.Y99Y99. | 21 | 15 | 27 | 63 | Yes | Yes | Yes |
| | | | | | R99R99.G99G99. | T99T99.S99S99. | C99C99.R99R99. | | | | | | | |
| | | | | | L99L99.V99V99. | C99C99. | Q99Q99.L99L99. | | | | | | | |
| | | | | | Y95B00. | | L99L99.V97B00. | | | | | | | |
| | | | | | | | T99T99. | | | | | | | |
| 270 | 46 | NSIVCCA | FRGTV | AHPSAD | N97B00.S99S99. | F99F99.R99R99. | A99A99.H99H99. | 21 | 15 | 18 | 54 | Yes | Yes | Yes |
| | | | | | I99I99.V99V99. | G99G99.T99T99. | P99P99.S99S99. | | | | | | | |
| | | | | | C99C99.C99C99. | V99V99. | A99A99.D99D99. | | | | | | | |
| | | | | | A99A99. | | | | | | | | | |
| 314 | 46 | HYPDARW | LATVV | THYSPYFSRC | H99H99.Y99Y99. | L99L99.A97B00. | T99T99.H99H99. | 21 | 15 | 30 | 66 | Yes | Yes | Yes |
| | | | | | P99P99.D97B00. | T99T99.V97B00. | Y99Y99.S99S99. | | | | | | | |
| | | | | | A99A99.R99R99. | V99V99. | P99P99.Y99Y99. | | | | | | | |
| | | | | | W99W99. | | F99F99.S99S99. | | | | | | | |
| | | | | | | | R97B00.C99C99. | | | | | | | |
| 64 | 46 | SCWQWHR | RSFSD | FCVGYH | S99S99.C99C99. | R99R99.S99S99. | F99F99.C99C99. | 21 | 15 | 18 | 54 | Yes | Yes | Yes |
| | | | | | W99W99.Q99Q99. | F99F99.S99S99. | V99V99.G99G99. | | | | | | | |
| | | | | | W99W99.H97B00. | D99D99. | Y99Y99.H99H99. | | | | | | | |
| | | | | | R99R99. | | | | | | | | | |
| 200 | 45 | NPVDIQC | HRLMA | ESTSYF | N99N99.P97B00. | H99H99.R99R99. | B00 | 21 | 15 | 0 | 36 | Yes | Yes | Yes |
| | | | | | V99V99.D99D99. | L99L99.M99M99. | | | | | | | | |
| | | | | | I99I99.Q97B00. | A99A99. | | | | | | | | |
| | | | | | C99C99. | | | | | | | | | |
| 3 | 45 | VRGLVLN | RSG | PIHFLNRTV | V99V99.R99R99. | R99R99.S99S99. | P99P99.I99I99. | 21 | 9 | 27 | 57 | Yes | Yes | Yes |
| | | | | | G99G99.L99L99. | G99G99. | H99H99.F99F99. | | | | | | | |
| | | | | | V99V99.L99L99. | | L99L99.N99N99. | | | | | | | |
| | | | | | N97B00. | | R99R99.T99T99. | | | | | | | |
| | | | | | | | V99V99. | | | | | | | |
| 382 | 45 | ITHSLVY | NNGKW | VFLSGDSVCH | I97B00.T99T99. | N99N99.N99N99. | V99V99.F88B00. | 21 | 15 | 30 | 66 | Yes | Yes | Yes |
| | | | | | H99H99.S99S99. | G99G99.K99K99. | L88B00.S99S99. | | | | | | | |
| | | | | | L99L99.V99V99. | W99W99. | G88B00.D88B00. | | | | | | | |
| | | | | | Y99Y99. | | S88B00.V88B00. | | | | | | | |
| | | | | | | | C88B00.H88B00. | | | | | | | |
| 408 | 45 | MGTSLSG | CGALL | YNLSTV | M97B00.G99G99. | C99C99.G99G99. | B00 | 21 | 15 | 0 | 36 | Yes | Yes | Yes |
| | | | | | T97B00.S99S99. | A99A99.L99L99. | | | | | | | | |
| | | | | | L99L99.S99S99. | L99L99. | | | | | | | | |
| | | | | | G99G99. | | | | | | | | | |
| 108 | 44 | VNCLVW | FWDGR | GPDFVV | V99V99.N97B00. | F99F99.W99W99. | G88B00.P88B00. | 18 | 15 | 18 | 51 | Yes | Yes | Yes |
| | | | | | C99C99.L99L99. | D99D99.G99G99. | D88B00.F88B00. | | | | | | | |
| | | | | | V99V99.W99W99. | R99R99. | V88B00.V99V99. | | | | | | | |
| 276 | 44 | APCVLLS | RGVHH | HEGACRRLLDQSA | A99A99.P99P99. | R99R99.G99G99. | H99H99.E99E99. | 21 | 15 | 39 | 75 | Yes | Yes | Yes |
| | | | | | C99C99.V99V99. | V99V99.H99H99. | G99G99.A99A99. | | | | | | | |
| | | | | | L99L99.L99L99. | H99H99. | C99C99.R99R99. | | | | | | | |
| | | | | | S99S99. | | R99R99.L99L99. | | | | | | | |
| | | | | | | | L99L99.D99D99. | | | | | | | |
| | | | | | | | Q99Q99.S99S99. | | | | | | | |
| | | | | | | | A99A99. | | | | | | | |
| 424 | 44 | AMTGVYM | PGTGF | AHLISK | A99A99.M99M99. | P99P99.G99G99. | A99A99.H99H99. | 21 | 15 | 18 | 54 | Yes | Yes | Yes |
| | | | | | T97B00.G99G99. | T99T99.G99G99. | L99L99.I97B00. | | | | | | | |
| | | | | | V99V99.Y99Y99. | F99F99. | S99S99.K99K99. | | | | | | | |
| | | | | | M99M99. | | | | | | | | | |
| 142 | 43 | LHHRVVF | PACSD | AVCLDHLVIH | L99L99.H93B00. | P99P99.A99A99. | A99A99.V93B00. | 21 | 15 | 30 | 66 | Yes | Yes | Yes |
| | | | | | H99H99.R99R99. | C99C99.S99S99. | C93B00.L93B00. | | | | | | | |
| | | | | | V99V99.V99V99. | D99D99. | D93B00.H93B00. | | | | | | | |
| | | | | | F99F99. | | L93B00.V93B00. | | | | | | | |
| | | | | | | | I93B00.H93B00. | | | | | | | |
| 425 | 43 | EMSRIVY | SGACL | IKIQGEELA | E99E99.M99M99. | S99S99.G97B00. | I99I99.K99K99. | 21 | 15 | 27 | 63 | Yes | Yes | Yes |
| | | | | | S97B00.R99R99. | A99A99.C99C99. | I99I99.Q99Q99. | | | | | | | |
| | | | | | I99I99.V99V99. | L99L99. | G99G99.E97B00. | | | | | | | |
| | | | | | Y99Y99. | | E99E99.L99L99. | | | | | | | |
| | | | | | | | A99A99. | | | | | | | |
| 237 | 42 | R | PHNLG | IHYGAK | R99R99. | P99P99.H99H99. | I99I99.H99H99. | 3 | 15 | 18 | 36 | Yes | Yes | Yes |
| | | | | | | N99N99.L99L99. | Y99Y99.G99G99. | | | | | | | |
| | | | | | | G99G99. | A99A99.K99K99. | | | | | | | |
| 375 | 42 | RLPCVVF | SGSIV | HTFLST | R99R99.L99L99. | S99S99.G99G99. | H99H99.T99T99. | 21 | 15 | 18 | 54 | Yes | Yes | Yes |
| | | | | | P99P99.C99C99. | S99S99.I99I99. | F99F99.L99L99. | | | | | | | |
| | | | | | V99V99.V99V99. | V99V99. | S99S99.T99T99. | | | | | | | |
| | | | | | F99F99. | | | | | | | | | |
| 155 | 41 | RHFHIVA | GWQGI | YIFNQSNCCF | R99R99.H99H99. | G99G99.W99W99. | Y99Y99.I97B00. | 21 | 15 | 30 | 66 | Yes | Yes | Yes |
| | | | | | F99F99.H99H99. | Q99Q99.G99G99. | F97B00.N97B00. | | | | | | | |
| | | | | | I97B00.V99V99. | I99I99. | Q97B00.S97B00. | | | | | | | |
| | | | | | A99A99. | | N97B00.C97B00. | | | | | | | |
| | | | | | | | C97B00.F97B00. | | | | | | | |
| 81 | 41 | RNSGCVF | ACDSI | SYFGLRVVS | R97B00.N97B00. | A99A99.C99C99. | S87B00.Y95B00. | 21 | 15 | 27 | 63 | Yes | Yes | Yes |
| | | | | | S99S99.G99G99. | D99D99.S99S99. | F95B00.G87B00. | | | | | | | |
| | | | | | C99C99.V99V99. | I99I99. | L92B00.R87B00. | | | | | | | |
| | | | | | F99F99. | | V87B00.V87B00. | | | | | | | |
| | | | | | | | S87B00. | | | | | | | |
| 177 | 40 | GNFDVFS | TFSRG | LCCPVCVIV | G97B00.N99N99. | T99T99.F99F99. | L99L99.C99C99. | 21 | 15 | 27 | 63 | Yes | Yes | Yes |
| | | | | | F99F99.D99D99. | S99S99.R99R99. | C99C99.P99P99. | | | | | | | |
| | | | | | V99V99.F99F99. | G99G99. | V99V99.C99C99. | | | | | | | |
| | | | | | S99S99. | | V99V99.I99I99. | | | | | | | |
| | | | | | | | V99V99. | | | | | | | |
| 242 | 40 | NSNVVYA | TFLGN | IIPYASRLHY | N95B00.S97B00. | T99T99.F99F99. | I90B00.I90B00. | 21 | 15 | 30 | 66 | Yes | Yes | Yes |
| | | | | | N99N99.V99V99. | L99L99.G99G99. | P99P99.Y90B00. | | | | | | | |
| | | | | | V99V99.Y99Y99. | N99N99. | A90B00.S90B00. | | | | | | | |
| | | | | | A99A99. | | R90B00.L90B00. | | | | | | | |
| | | | | | | | H90B00.Y90B00. | | | | | | | |
| 131 | 39 | DKCAYIM | GVTIN | LTNECL | D97B00.K97B00. | G99G99.V99V99. | L99L99.T99T99. | 21 | 15 | 18 | 54 | Yes | Yes | Yes |
| | | | | | C99C99.A99A99. | T99T99.I99I99. | N99N99.E99E99. | | | | | | | |
| | | | | | Y99Y99.I97B00. | N99N99. | C99C99.L99L99. | | | | | | | |
| | | | | | M97B00. | | | | | | | | | |
| 317 | 39 | AGCFVSN | FRSLQ | HTPDAS | A99A99.G99G99. | F99F99.R99R99. | H99H99.T99T99. | 21 | 15 | 18 | 54 | Yes | Yes | Yes |
| | | | | | C99C99.F99F99. | S99S99.L99L99. | P99P99.D99D99. | | | | | | | |
| | | | | | V97B00.S99S99. | Q99Q99. | A99A99.S99S99. | | | | | | | |
| | | | | | N94B00. | | | | | | | | | |
| 384 | 38 | YYEALRG | IDSYS | KHFNTGLQRWIYV | Y99Y99.Y99Y99. | I99I99.D97B00. | K99K99.H99H99. | 21 | 15 | 39 | 75 | Yes | Yes | Yes |
| | | | | | E99E99.A99A99. | S99S99.Y99Y99. | F99F99.N99N99. | | | | | | | |
| | | | | | L99L99.R99R99. | S99S99. | T97B00.G99G99. | | | | | | | |
| | | | | | G99G99. | | L99L99.Q99Q99. | | | | | | | |
| | | | | | | | R99R99.W99W99. | | | | | | | |
| | | | | | | | I99I99.Y99Y99. | | | | | | | |
| | | | | | | | V99V99. | | | | | | | |
| 470 | 38 | VTTKQVY | MVLGS | VCLVGTSCV | V99V99.T99T99. | M99M99.V99V99. | V99V99.C99C99. | 21 | 15 | 27 | 63 | Yes | Yes | Yes |
| | | | | | T99T99.K99K99. | L99L99.G99G99. | L99L99.V99V99. | | | | | | | |
| | | | | | Q99Q99.V99V99. | S99S99. | G99G99.T99T99. | | | | | | | |
| | | | | | Y97B00. | | S99S99.C97B00. | | | | | | | |
| | | | | | | | V97B00. | | | | | | | |
| 630 | 38 | KVFLTVW | NDGHT | VSFQPRHPVV | K97B00.V99V99. | N99N99.D99D99. | V99V99.S99S99. | 21 | 15 | 30 | 66 | Yes | Yes | Yes |
| | | | | | F99F99.L99L99. | G99G99.H99H99. | F99F99.Q99Q99. | | | | | | | |
| | | | | | T99T99.V99V99. | T99T99. | P99P99.R99R99. | | | | | | | |
| | | | | | W99W99. | | H99H99.P99P99. | | | | | | | |
| | | | | | | | V99V99.V99V99. | | | | | | | |
| 96 | 38 | RARGKVF | CSGVD | IFYDRTDRLS | R97B00.A97B00. | C99C99.S99S99. | I99I99.F99F99. | 21 | 15 | 30 | 66 | Yes | Yes | Yes |
| | | | | | R97B00.G97B00. | G99G99.V99V99. | Y99Y99.D99D99. | | | | | | | |
| | | | | | K97B00.V97B00. | D99D99. | R99R99.T99T99. | | | | | | | |
| | | | | | F97B00. | | D99D99.R99R99. | | | | | | | |
| | | | | | | | L99L99.S99S99. | | | | | | | |
| 111 | 37 | LESLCG | VGAIR | WNYDCLGIS | L99L99.E99E99. | V99V99.G99G99. | B00 | 18 | 15 | 0 | 33 | Yes | Yes | Yes |
| | | | | | S99S99.L99L99. | A99A99.I99I99. | | | | | | | | |
| | | | | | C99C99.G99G99. | R99R99. | | | | | | | | |
| 32 | 37 | RLMNEVF | RSAGT | AFYISFMVY | R99R99.L99L99. | R99R99.S99S99. | A99A99.F99F99. | 21 | 15 | 27 | 63 | Yes | Yes | Yes |
| | | | | | M99M99.N99N99. | A99A99.G99G99. | Y99Y99.I99I99. | | | | | | | |
| | | | | | E99E99.V99V99. | T99T99. | S99S99.F99F99. | | | | | | | |
| | | | | | F99F99. | | M99M99.V97B00. | | | | | | | |
| | | | | | | | Y99Y99. | | | | | | | |
| 436 | 37 | IYLEAYG | LYSFR | LQPTPSGWRE | I99I99.Y94B00. | L99L99.Y99Y99. | L99L99.Q99Q99. | 21 | 15 | 30 | 66 | Yes | Yes | Yes |
| | | | | | L99L99.E99E99. | S99S99.F99F99. | P99P99.T99T99. | | | | | | | |
| | | | | | A99A99.Y99Y99. | R99R99. | P99P99.S99S99. | | | | | | | |
| | | | | | G99G99. | | G99G99.W99W99. | | | | | | | |
| | | | | | | | R99R99.E99E99. | | | | | | | |
| 614 | 37 | SCIRWLS | EYVKF | LENPHIASKY | S99S99.C99C99. | E99E99.Y99Y99. | L97B00.E97B00. | 21 | 15 | 30 | 66 | Yes | Yes | Yes |
| | | | | | I99I99.R99R99. | V99V99.K99K99. | N97B00.P97B00. | | | | | | | |
| | | | | | W99W99.L99L99. | F99F99. | H97B00.I97B00. | | | | | | | |
| | | | | | S99S99. | | A97B00.S97B00. | | | | | | | |
| | | | | | | | K97B00.Y97B00. | | | | | | | |
| 70 | 37 | TVIPYS | CMFLH | PVPIPEESIT | T97B00.V97B00. | C99C99.M99M99. | P99P99.V86B00. | 18 | 15 | 30 | 63 | Yes | Yes | Yes |
| | | | | | I94B00.P97B00. | F99F99.L99L99. | P86B00.I86B00. | | | | | | | |
| | | | | | Y97B00.S94B00. | H99H99. | P86B00.E86B00. | | | | | | | |
| | | | | | | | E86B00.S86B00. | | | | | | | |
| | | | | | | | I99I99.T86B00. | | | | | | | |
| 76 | 37 | RTYRMVF | VCSSV | VSLLRTITAH | R99R99.T99T99. | V99V99.C99C99. | V99V99.S37B00. | 21 | 15 | 13 | 49 | Yes | Yes | Yes |
| | | | | | Y99Y99.R99R99. | S97B00.S99S99. | L37B00.L37B00. | | | | | | | |
| | | | | | M99M99.V99V99. | V99V99. | R37B00.T37B00. | | | | | | | |
| | | | | | F99F99. | | I37B00.T70B00. | | | | | | | |
| | | | | | | | A37B00.H37B00. | | | | | | | |
| 109 | 36 | CVTRRYR | NVLTL | VDSTLFGV | C99C99.V99V99. | N99N99.V99V99. | V99V99.D99D99. | 21 | 15 | 24 | 60 | Yes | Yes | Yes |
| | | | | | T99T99.R99R99. | L99L99.T99T99. | S97B00.T97B00. | | | | | | | |
| | | | | | R99R99.Y99Y99. | L99L99. | L99L99.F99F99. | | | | | | | |
| | | | | | R99R99. | | G99G99.V97B00. | | | | | | | |
| 298 | 36 | IDFCYRF | PYAFR | DCPLFSLCF | I94B00.D99D99. | P99P99.Y99Y99. | D99D99.C99C99. | 21 | 15 | 27 | 63 | Yes | Yes | Yes |
| | | | | | F99F99.C99C99. | A97B00.F99F99. | P99P99.L99L99. | | | | | | | |
| | | | | | Y99Y99.R99R99. | R99R99. | F99F99.S97B00. | | | | | | | |
| | | | | | F99F99. | | L99L99.C97B00. | | | | | | | |
| | | | | | | | F99F99. | | | | | | | |
| 336 | 36 | MSE | DWRGC | SNPPISYRVD | M99M99.S99S99. | D99D99.W99W99. | S99S99.N99N99. | 9 | 15 | 30 | 54 | Yes | Yes | Yes |
| | | | | | E99E99. | R99R99.G99G99. | P99P99.P99P99. | | | | | | | |
| | | | | | | C99C99. | I99I99.S99S99. | | | | | | | |
| | | | | | | | Y99Y99.R99R99. | | | | | | | |
| | | | | | | | V99V99.D99D99. | | | | | | | |
| 352 | 36 | PECPIIY | HGIGE | VHYCHGQVF | P99P99.E99E99. | H99H99.G99G99. | V99V99.H99H99. | 21 | 15 | 27 | 63 | Yes | Yes | Yes |
| | | | | | C99C99.P99P99. | I99I99.G97B00. | Y99Y99.C99C99. | | | | | | | |
| | | | | | I99I99.I99I99. | E99E99. | H99H99.G99G99. | | | | | | | |
| | | | | | Y99Y99. | | Q99Q99.V99V99. | | | | | | | |
| | | | | | | | F99F99. | | | | | | | |
| 387 | 36 | SFMPFIR | NPRGT | QLVLVIEQW | S99S99.F99F99. | N99N99.P99P99. | Q99Q99.L99L99. | 21 | 15 | 27 | 63 | Yes | Yes | Yes |
| | | | | | M97B00.P99P99. | R99R99.G99G99. | V99V99.L99L99. | | | | | | | |
| | | | | | F99F99.I99I99. | T99T99. | V99V99.I99I99. | | | | | | | |
| | | | | | R99R99. | | E99E99.Q99Q99. | | | | | | | |
| | | | | | | | W99W99. | | | | | | | |
| 540 | 36 | VQPGVVY | ACDS | WCMSGVAIM | V99V99.Q99Q99. | A99A99.C99C99. | W99W99.C99C99. | 21 | 12 | 27 | 60 | Yes | Yes | Yes |
| | | | | | P99P99.G99G99. | D99D99.S99S99. | M99M99.S99S99. | | | | | | | |
| | | | | | V99V99.V99V99. | | G99G99.V99V99. | | | | | | | |
| | | | | | Y99Y99. | | A99A99.I99I99. | | | | | | | |
| | | | | | | | M99M99. | | | | | | | |
| 619 | 36 | LSIEGCQ | SSRWS | VTISIICHILHVH | L97B00.S99S99. | S99S99.S99S99. | V99V99.T99T99. | 21 | 15 | 39 | 75 | Yes | Yes | Yes |
| | | | | | I99I99.E99E99. | R99R99.W99W99. | I99I99.S99S99. | | | | | | | |
| | | | | | G99G99.C99C99. | S99S99. | I99I99.I99I99. | | | | | | | |
| | | | | | Q99Q99. | | C99C99.H99H99. | | | | | | | |
| | | | | | | | I99I99.L99L99. | | | | | | | |
| | | | | | | | H99H99.V99V99. | | | | | | | |
| | | | | | | | H99H99. | | | | | | | |
| 19 | 35 | SCGSVVY | SWGAA | AIFLKSSVVL | S94B00.C99C99. | S99S99.W99W99. | A99A99.I99I99. | 21 | 15 | 30 | 66 | Yes | Yes | Yes |
| | | | | | G99G99.S99S99. | G99G99.A99A99. | F99F99.L99L99. | | | | | | | |
| | | | | | V99V99.V97B00. | A99A99. | K99K99.S99S99. | | | | | | | |
| | | | | | Y99Y99. | | S99S99.V99V99. | | | | | | | |
| | | | | | | | V99V99.L99L99. | | | | | | | |
| 121 | 35 | YICHVTM | PVYND | ISLQSDFSMH | Y97B00.I94B00. | P94B00.V91B00. | I99I99.S97B00. | 21 | 15 | 30 | 66 | Yes | Yes | Yes |
| | | | | | C97B00.H97B00. | Y94B00.N94B00. | L99L99.Q97B00. | | | | | | | |
| | | | | | V97B00.T97B00. | D94B00. | S97B00.D97B00. | | | | | | | |
| | | | | | M94B00. | | F94B00.S97B00. | | | | | | | |
| | | | | | | | M97B00.H97B00. | | | | | | | |
| 135 | 35 | QSGKGCR | TGGVG | WTWSDHYYLS | Q99Q99.S97B00. | T99T99.G99G99. | W99W99.T99T99. | 21 | 15 | 30 | 66 | Yes | Yes | Yes |
| | | | | | G99G99.K99K99. | G99G99.V99V99. | W99W99.S99S99. | | | | | | | |
| | | | | | G99G99.C99C99. | G99G99. | D99D99.H99H99. | | | | | | | |
| | | | | | R99R99. | | Y99Y99.Y99Y99. | | | | | | | |
| | | | | | | | L99L99.S99S99. | | | | | | | |
| 281 | 35 | SVSAVF | KTIGS | FQYQDGSVF | S99S99.I99I99. | K99K99.T99T99. | F99F99.Q99Q99. | 21 | 15 | 27 | 63 | Yes | Yes | Yes |
| | | | | | V99V99.S99S99. | I99I99.G99G99. | Y99Y99.Q97B00. | | | | | | | |
| | | | | | A94B00.V99V99. | S99S99. | D99D99.G99G99. | | | | | | | |
| | | | | | F99F99. | | S99S99.V99V99. | | | | | | | |
| | | | | | | | F99F99. | | | | | | | |
| 400 | 35 | AWEDLVC | GFTRT | VDNGLC | A99A99.W99W99. | G97B00.F99F99. | V99V99.D99D99. | 21 | 15 | 18 | 54 | Yes | Yes | Yes |
| | | | | | E99E99.D99D99. | T99T99.R99R99. | N99N99.G99G99. | | | | | | | |
| | | | | | L99L99.V99V99. | T99T99. | L99L99.C99C99. | | | | | | | |
| | | | | | C99C99. | | | | | | | | | |
| 542 | 35 | VLGIVGG | GGARQ | SSRFGA | V99V99.L99L99. | G99G99.G99G99. | S99S99.S97B00. | 21 | 15 | 18 | 54 | Yes | Yes | Yes |
| | | | | | G99G99.I99I99. | A99A99.R99R99. | R99R99.F99F99. | | | | | | | |
| | | | | | V99V99.G99G99. | Q99Q99. | G99G99.A99A99. | | | | | | | |
| | | | | | G99G99. | | | | | | | | | |
| 74 | 35 | YRVYYAL | GHSDA | LHLLGR | Y99Y99.R99R99. | G97B00.H99H99. | L97B00.H97B00. | 21 | 15 | 18 | 54 | Yes | Yes | Yes |
| | | | | | V99V99.Y99Y99. | S99S99.D99D99. | L97B00.L97B00. | | | | | | | |
| | | | | | Y99Y99.A99A99. | A99A99. | G97B00.R94B00. | | | | | | | |
| | | | | | L99L99. | | | | | | | | | |
| 221 | 34 | LGCVWGQ | AYSGS | LIDYTRDDGWQSV | L94B00.G94B00. | A94B00.Y94B00. | L99L99.I94B00. | 21 | 15 | 39 | 75 | Yes | Yes | Yes |
| | | | | | C99C99.V91B00. | S94B00.G94B00. | D94B00.Y99Y99. | | | | | | | |
| | | | | | W99W99.G99G99. | S99S99. | T94B00.R94B00. | | | | | | | |
| | | | | | Q91B00. | | D99D99.D94B00. | | | | | | | |
| | | | | | | | G94B00.W99W99. | | | | | | | |
| | | | | | | | Q94B00.S94B00. | | | | | | | |
| | | | | | | | V94B00. | | | | | | | |
| 248 | 34 | WFGRLVY | ADGCT | ATFVKYEVL | W99W99.F99F99. | A99A99.D99D99. | A99A99.T99T99. | 21 | 15 | 27 | 63 | Yes | Yes | Yes |
| | | | | | G99G99.R99R99. | G97B00.C99C99. | F99F99.V99V99. | | | | | | | |
| | | | | | L99L99.V99V99. | T99T99. | K99K99.Y99Y99. | | | | | | | |
| | | | | | Y99Y99. | | E99E99.V99V99. | | | | | | | |
| | | | | | | | L99L99. | | | | | | | |
| 319 | 34 | SFGL | FPFGL | VARR | S99S99.F99F99. | F99F99.P99P99. | V99V99.A99A99. | 12 | 15 | 12 | 39 | Yes | Yes | Yes |
| | | | | | G99G99.L99L99. | F99F99.G99G99. | R99R99.R99R99. | | | | | | | |
| | | | | | | L99L99. | | | | | | | | |
| 105 | 33 | LDVRYFV | FYSCV | NYFDAM | L99L99.D96B00. | F99F99.Y99Y99. | N78B00.Y78B00. | 21 | 15 | 12 | 48 | Yes | Yes | Yes |
| | | | | | V99V99.R99R99. | S99S99.C99C99. | F75B00.D75B00. | | | | | | | |
| | | | | | Y99Y99.F99F99. | V99V99. | A75B00.M75B00. | | | | | | | |
| | | | | | V99V99. | | | | | | | | | |
| 160 | 33 | VIGHFVY | ADSSR | ATLISNVGVK | V99V99.I99I99. | A99A99.D99D99. | A96B00.T96B00. | 21 | 15 | 30 | 66 | Yes | Yes | Yes |
| | | | | | G96B00.H99H99. | S99S99.S99S99. | L96B00.I96B00. | | | | | | | |
| | | | | | F99F99.V99V99. | R99R99. | S96B00.N96B00. | | | | | | | |
| | | | | | Y99Y99. | | V96B00.G96B00. | | | | | | | |
| | | | | | | | V96B00.K96B00. | | | | | | | |
| 258 | 33 | NFVCNHQ | WSVAV | TLTVITRCR | N96B00.F99F99. | W99W99.S99S99. | T99T99.L99L99. | 21 | 15 | 20 | 56 | Yes | Yes | Yes |
| | | | | | V99V99.C99C99. | V99V99.A99A99. | T63S36.V60L36. | | | | | | | |
| | | | | | N99N99.H99H99. | V99V99. | I63S36.T60S36. | | | | | | | |
| | | | | | Q96B00. | | R63S36.C63P36. | | | | | | | |
| | | | | | | | R63K36. | | | | | | | |
| 274 | 33 | VHPHIFG | LWASD | TAFLGCDMLL | V99V99.H96B00. | L99L99.W99W99. | T93B00.A93B00. | 21 | 15 | 30 | 66 | Yes | Yes | Yes |
| | | | | | P99P99.H99H99. | A99A99.S99S99. | F93B00.L99L99. | | | | | | | |
| | | | | | I99I99.F99F99. | D96B00. | G93B00.C93B00. | | | | | | | |
| | | | | | G99G99. | | D90B00.M93B00. | | | | | | | |
| | | | | | | | L93B00.L93B00. | | | | | | | |
| 460 | 33 | VLGLLRL | CLAHG | VTRFDA | V96B00.L96B00. | C99C99.L96B00. | V99V99.T99T99. | 21 | 15 | 18 | 54 | Yes | Yes | Yes |
| | | | | | G96B00.L96B00. | A99A99.H99H99. | R99R99.F99F99. | | | | | | | |
| | | | | | L96B00.R96B00. | G99G99. | D96B00.A99A99. | | | | | | | |
| | | | | | L96B00. | | | | | | | | | |
| 592 | 33 | ITHSCVY | HDVAS | VSLTHGGIV | I96B00.T99T99. | H99H99.D99D99. | V72B00.S72B00. | 21 | 15 | 18 | 54 | Yes | Yes | Yes |
| | | | | | H99H99.S99S99. | V99V99.A99A99. | L72B00.T72B00. | | | | | | | |
| | | | | | C99C99.V99V99. | S99S99. | H72B00.G72B00. | | | | | | | |
| | | | | | Y99Y99. | | G72B00.I72B00. | | | | | | | |
| | | | | | | | V72B00. | | | | | | | |
| 685 | 33 | NGGHVFA | PDTMC | TQWLVEEVM | N90B00.G99G99. | P99P99.D99D99. | T99T99.Q99Q99. | 21 | 15 | 27 | 63 | Yes | Yes | Yes |
| | | | | | G99G99.H99H99. | T99T99.M99M99. | W99W99.L99L99. | | | | | | | |
| | | | | | V99V99.F99F99. | C99C99. | V99V99.E99E99. | | | | | | | |
| | | | | | A99A99. | | E99E99.V99V99. | | | | | | | |
| | | | | | | | M99M99. | | | | | | | |
| 114 | 32 | SIPRVVI | DLGRG | YIFHYTSSVV | S99S99.I99I99. | D99D99.L99L99. | Y99Y99.I99I99. | 21 | 15 | 30 | 66 | Yes | Yes | Yes |
| | | | | | P99P99.R99R99. | G99G99.R96B00. | F99F99.H99H99. | | | | | | | |
| | | | | | V99V99.V99V99. | G99G99. | Y99Y99.T99T99. | | | | | | | |
| | | | | | I99I99. | | S99S99.S96B00. | | | | | | | |
| | | | | | | | V99V99.V99V99. | | | | | | | |
| 136 | 32 | CLRGICW | YNCVR | SRFDSF | C99C99.L99L99. | Y99Y99.N99N99. | S99S99.R99R99. | 21 | 15 | 18 | 54 | Yes | Yes | Yes |
| | | | | | R99R99.G99G99. | C99C99.V99V99. | F99F99.D99D99. | | | | | | | |
| | | | | | I99I99.C99C99. | R99R99. | S99S99.F99F99. | | | | | | | |
| | | | | | W99W99. | | | | | | | | | |
| 31 | 32 | HLGIEVA | RFPEN | SMLADNGVTS | H96B00.L96B00. | R96B00.F96B00. | S99S99.M99M99. | 21 | 15 | 30 | 66 | Yes | Yes | Yes |
| | | | | | G96B00.I96B00. | P96B00.E96B00. | L99L99.A99A99. | | | | | | | |
| | | | | | E96B00.V96B00. | N96B00. | D99D99.N99N99. | | | | | | | |
| | | | | | A96B00. | | G99G99.V99V99. | | | | | | | |
| | | | | | | | T99T99.S99S99. | | | | | | | |
| 447 | 32 | KCSLIVY | RDSRG | CYFGASYIS | K99K99.C99C99. | R99R99.D99D99. | C71B00.Y71B00. | 21 | 15 | 19 | 55 | Yes | Yes | Yes |
| | | | | | S96B00.L99L99. | S99S99.R99R99. | F71B00.G71B00. | | | | | | | |
| | | | | | I99I99.V99V99. | G99G99. | A87B00.S71B00. | | | | | | | |
| | | | | | Y99Y99. | | Y71B00.I71B00. | | | | | | | |
| | | | | | | | S71B00. | | | | | | | |
| 59 | 32 | GGLFDMF | PKQHN | LRPPSC | G99G99.G99G99. | P96B00.K99K99. | B00 | 21 | 15 | 0 | 36 | Yes | Yes | Yes |
| | | | | | L99L99.F99F99. | Q99Q99.H99H99. | | | | | | | | |
| | | | | | D99D99.M93B00. | N99N99. | | | | | | | | |
| | | | | | F99F99. | | | | | | | | | |
| 604 | 32 | LFLRNVY | HSSGD | VYLLQACIVK | L99L99.F99F99. | H99H99.S99S99. | V99V99.Y99Y99. | 21 | 15 | 30 | 66 | Yes | Yes | Yes |
| | | | | | L99L99.R99R99. | S99S99.G99G99. | L99L99.L99L99. | | | | | | | |
| | | | | | N99N99.V99V99. | D99D99. | Q99Q99.A99A99. | | | | | | | |
| | | | | | Y99Y99. | | C99C99.I99I99. | | | | | | | |
| | | | | | | | V99V99.K99K99. | | | | | | | |
| 33 | 31 | IRCTLGW | LAAQV | PYWYFGEVR | I96B00.R99R99. | L99L99.A99A99. | P99P99.Y99Y99. | 21 | 15 | 27 | 63 | Yes | Yes | Yes |
| | | | | | C93B00.T96B00. | A99A99.Q99Q99. | W99W99.Y99Y99. | | | | | | | |
| | | | | | L93B00.G96B00. | V99V99. | F99F99.G96B00. | | | | | | | |
| | | | | | W96B00. | | E99E99.V99V99. | | | | | | | |
| | | | | | | | R96B00. | | | | | | | |
| 44 | 31 | FIYRLVY | RAAEN | YSLGYLADIS | F96B00.I96B00. | R99R99.A99A99. | Y96B00.S93B00. | 21 | 15 | 30 | 66 | Yes | Yes | Yes |
| | | | | | Y96B00.R96B00. | A99A99.E99E99. | L96B00.G99G99. | | | | | | | |
| | | | | | L96B00.V96B00. | N99N99. | Y96B00.L96B00. | | | | | | | |
| | | | | | Y96B00. | | A96B00.D96B00. | | | | | | | |
| | | | | | | | I96B00.S96B00. | | | | | | | |
| 450 | 31 | INVRVCC | ALYIG | CLPNAH | I96B00.N99N99. | A99A99.L99L99. | C61S38.L61H38. | 21 | 15 | 13 | 49 | Yes | Yes | Yes |
| | | | | | V99V99.R99R99. | Y99Y99.I99I99. | P99P99.N61S38. | | | | | | | |
| | | | | | V99V99.C99C99. | G99G99. | A61S38.H58T38. | | | | | | | |
| | | | | | C99C99. | | | | | | | | | |
| 477 | 31 | SINGGVY | RAGCWW | AFLRGNRVI | S99S99.I99I99. | R99R99.A99A99. | A99A99.F99F99. | 21 | 15 | 27 | 63 | Yes | Yes | Yes |
| | | | | | N96B00.G99G99. | G99G99.C99C99. | L99L99.R99R99. | | | | | | | |
| | | | | | G99G99.V99V99. | W99W99. | G99G99.N99N99. | | | | | | | |
| | | | | | Y96B00. | | R99R99.V99V99. | | | | | | | |
| | | | | | | | I99I99. | | | | | | | |
| 184 | 30 | FLSSVWF | TCHMD | RYGWCT | F99F99.L99L99. | T99T99.C99C99. | R70B00.Y70B00. | 21 | 15 | 13 | 49 | Yes | Yes | Yes |
| | | | | | S99S99.S99S99. | H99H99.M99M99. | G96B00.W70B00. | | | | | | | |
| | | | | | V99V99.W99W99. | D99D99. | C70B00.T70B00. | | | | | | | |
| | | | | | F99F99. | | | | | | | | | |
| 196 | 30 | FNCCDVQ | GVSCE | LLGYSSEVRW | F99F99.N96B00. | G99G99.V99V99. | L99L99.L99L99. | 21 | 15 | 30 | 66 | Yes | Yes | Yes |
| | | | | | C99C99.C99C99. | S99S99.C99C99. | G99G99.Y99Y99. | | | | | | | |
| | | | | | D99D99.V99V99. | E99E99. | S99S99.S99S99. | | | | | | | |
| | | | | | Q99Q99. | | E99E99.V99V99. | | | | | | | |
| | | | | | | | R99R99.W99W99. | | | | | | | |
| 204 | 30 | GIFVYGG | GVTAN | LHDTHLKAVDSN | G99G99.I99I99. | G99G99.V99V99. | L99L99.H99H99. | 21 | 15 | 36 | 72 | Yes | Yes | Yes |
| | | | | | F99F99.V99V99. | T99T99.A99A99. | D99D99.T99T99. | | | | | | | |
| | | | | | Y99Y99.G99G99. | N99N99. | H99H99.L99L99. | | | | | | | |
| | | | | | G99G99. | | K99K99.A99A99. | | | | | | | |
| | | | | | | | V96B00.D99D99. | | | | | | | |
| | | | | | | | S99S99.N99N99. | | | | | | | |
| 245 | 30 | GRSRQSD | ISCCD | FWRGRIDCSIMRL | G99G99.R99R99. | I99I99.S99S99. | F99F99.W99W99. | 21 | 15 | 39 | 75 | Yes | Yes | Yes |
| | | | | | S99S99.R99R99. | C99C99.C99C99. | R99R99.G99G99. | | | | | | | |
| | | | | | Q99Q99.S99S99. | D99D99. | R99R99.I99I99. | | | | | | | |
| | | | | | D99D99. | | D99D99.C99C99. | | | | | | | |
| | | | | | | | S99S99.I99I99. | | | | | | | |
| | | | | | | | M99M99.R99R99. | | | | | | | |
| | | | | | | | L96B00. | | | | | | | |
| 356 | 30 | GCSGWR | RELVG | YVISEAYGSD | G99G99.C99C99. | R99R99.E99E99. | Y99Y99.V99V99. | 21 | 15 | 30 | 66 | Yes | Yes | Yes |
| | | | | | S99S99.G99G99. | L99L99.V99V99. | I99I99.S99S99. | | | | | | | |
| | | | | | I99I99.W99W99. | G99G99. | E99E99.A99A99. | | | | | | | |
| | | | | | R99R99. | | Y99Y99.G99G99. | | | | | | | |
| | | | | | | | S96B00.D99D99. | | | | | | | |
| 393 | 30 | GAEFVGC | IWYSS | AHLHLGAVC | G99G99.A99A99. | I99I99.W99W99. | B00 | 21 | 15 | 0 | 36 | Yes | Yes | Yes |
| | | | | | E99E99.F99F99. | Y99Y99.S99S99. | | | | | | | | |
| | | | | | V99V99.G99G99. | S96B00. | | | | | | | | |
| | | | | | C99C99. | | | | | | | | | |
| 101 | 29 | NIGILLV | TPNGV | HIVEPPRHVS | N99N99.I99I99. | T99T99.P99P99. | H96B00.I96B00. | 21 | 15 | 30 | 66 | Yes | Yes | Yes |
| | | | | | G99G99.I99I99. | N99N99.G99G99. | V96B00.E96B00. | | | | | | | |
| | | | | | L99L99.L99L99. | V99V99. | P96B00.P96B00. | | | | | | | |
| | | | | | V99V99. | | R96B00.H96B00. | | | | | | | |
| | | | | | | | V96B00.S93B00. | | | | | | | |
| 102 | 29 | R | PHNLG | LDFQNTQIDR | R99R99. | P96B00.H99H99. | L99L99.D99D99. | 3 | 15 | 30 | 48 | Yes | Yes | Yes |
| | | | | | | N99N99.L99L99. | F93B00.Q99Q99. | | | | | | | |
| | | | | | | G99G99. | N99N99.T96B00. | | | | | | | |
| | | | | | | | Q99Q99.I99I99. | | | | | | | |
| | | | | | | | D99D99.R99R99. | | | | | | | |
| 123 | 29 | VVGVIVV | DRERV | TTRFSY | V99V99.V99V99. | D96B00.R96B00. | T96B00.T99T99. | 21 | 15 | 18 | 54 | Yes | Yes | Yes |
| | | | | | G99G99.V96B00. | E96B00.R96B00. | R96B00.F99F99. | | | | | | | |
| | | | | | I96B00.V99V99. | V96B00. | S96B00.Y99Y99. | | | | | | | |
| | | | | | V96B00. | | | | | | | | | |
| 301 | 29 | VMMPGPT | RCVFG | FTSADT | V99V99.M96B00. | R99R99.C99C99. | F99F99.T99T99. | 21 | 15 | 18 | 54 | Yes | Yes | Yes |
| | | | | | M99M99.P99P99. | V99V99.F99F99. | S99S99.A99A99. | | | | | | | |
| | | | | | G99G99.P99P99. | G99G99. | D99D99.T99T99. | | | | | | | |
| | | | | | T99T99. | | | | | | | | | |
| 529 | 29 | IVGVCVG | VRTAD | HTRFGW | I86B00.V86B00. | V86B00.R99R99. | H99H99.T99T99. | 21 | 15 | 18 | 54 | Yes | Yes | Yes |
| | | | | | G99G99.V86B00. | T86B00.A86B00. | R99R99.F99F99. | | | | | | | |
| | | | | | C86B00.V86B00. | D86B00. | G86B00.W99W99. | | | | | | | |
| | | | | | G86B00. | | | | | | | | | |
| 641 | 29 | ERYRSVY | GGSVF | ASFFMGAVS | E99E99.R99R99. | G99G99.G99G99. | A99A99.S99S99. | 21 | 15 | 27 | 63 | Yes | Yes | Yes |
| | | | | | Y99Y99.R99R99. | S99S99.V99V99. | F96B00.F99F99. | | | | | | | |
| | | | | | S99S99.V99V99. | F99F99. | M99M99.G99G99. | | | | | | | |
| | | | | | Y99Y99. | | A99A99.V99V99. | | | | | | | |
| | | | | | | | S99S99. | | | | | | | |
| 167 | 28 | VVDRWVC | SWSST | TLTWHTVHGAHLE | V99V99.V99V99. | S99S99.W99W99. | T99T99.L99L99. | 21 | 15 | 39 | 75 | Yes | Yes | Yes |
| | | | | | D96B00.R99R99. | S99S99.S99S99. | T99T99.W99W99. | | | | | | | |
| | | | | | W99W99.V99V99. | T99T99. | H99H99.T99T99. | | | | | | | |
| | | | | | C99C99. | | V99V99.H99H99. | | | | | | | |
| | | | | | | | G99G99.A99A99. | | | | | | | |
| | | | | | | | H96B00.L99L99. | | | | | | | |
| | | | | | | | E99E99. | | | | | | | |
| 171 | 28 | NIVGF | ASLSG | MTLHANLCIS | N96B00.I99I99. | A99A99.S99S99. | M99M99.T99T99. | 21 | 15 | 30 | 66 | Yes | Yes | Yes |
| | | | | | V99V99.G99G99. | L99L99.S99S99. | L99L99.H99H99. | | | | | | | |
| | | | | | C99C99.V99V99. | G99G99. | A99A99.N99N99. | | | | | | | |
| | | | | | F99F99. | | L99L99.C99C99. | | | | | | | |
| | | | | | | | I99I99.S99S99. | | | | | | | |
| 25 | 28 | AGLCMYQ | YPCFD | TLSVYGFQLR | A99A99.G99G99. | Y99Y99.P99P99. | T96B00.L96B00. | 21 | 15 | 30 | 66 | Yes | Yes | Yes |
| | | | | | L99L99.C99C99. | C99C99.F99F99. | S96B00.V96B00. | | | | | | | |
| | | | | | M99M99.Y99Y99. | D99D99. | Y96B00.G96B00. | | | | | | | |
| | | | | | Q96B00. | | F96B00.Q96B00. | | | | | | | |
| | | | | | | | L99L99.R96B00. | | | | | | | |
| 364 | 28 | IMPIISW | FHLDR | FTRFRH | I96B00.M99M99. | F99F99.H99H99. | F99F99.T99T99. | 21 | 15 | 18 | 54 | Yes | Yes | Yes |
| | | | | | P99P99.I99I99. | L99L99.D99D99. | R99R99.F99F99. | | | | | | | |
| | | | | | I99I99.S99S99. | R99R99. | R99R99.H99H99. | | | | | | | |
| | | | | | W99W99. | | | | | | | | | |
| 493 | 28 | LFTRVVF | ANACS | YIFTESGAVI | L99L99.F99F99. | A99A99.N99N99. | Y67B00.I67B00. | 21 | 15 | 20 | 56 | Yes | Yes | Yes |
| | | | | | T99T99.R99R99. | A99A99.C99C99. | F67B00.T67B00. | | | | | | | |
| | | | | | V99V99.V99V99. | S99S99. | E67B00.S67B00. | | | | | | | |
| | | | | | F99F99. | | G78B00.A67B00. | | | | | | | |
| | | | | | | | V78B00.I67B00. | | | | | | | |
| 495 | 28 | VCNRLVF | CGNRC | ISLSLDSCLQ | V99V99.C99C99. | C99C99.G99G99. | I99I99.S99S99. | 21 | 15 | 30 | 66 | Yes | Yes | Yes |
| | | | | | N96B00.R99R99. | N99N99.R99R99. | L99L99.S99S99. | | | | | | | |
| | | | | | L99L99.V99V99. | C99C99. | L99L99.D99D99. | | | | | | | |
| | | | | | F99F99. | | S99S99.C99C99. | | | | | | | |
| | | | | | | | L99L99.Q99Q99. | | | | | | | |
| 52 | 28 | GSVTFVH | NHSCR | YGSDMCSLLS | G99G99.S99S99. | N99N99.H99H99. | Y99Y99.G99G99. | 21 | 15 | 30 | 66 | Yes | Yes | Yes |
| | | | | | V99V99.T99T99. | S99S99.C99C99. | S99S99.D99D99. | | | | | | | |
| | | | | | F99F99.V99V99. | R99R99. | M99M99.C99C99. | | | | | | | |
| | | | | | H99H99. | | S99S99.L99L99. | | | | | | | |
| | | | | | | | L99L99.S99S99. | | | | | | | |
| 580 | 28 | VAGHLSR | PSILF | LPKLT | V99V99.A99A99. | P99P99.S99S99. | L99L99.P99P99. | 21 | 15 | 15 | 51 | Yes | Yes | Yes |
| | | | | | G99G99.H99H99. | I99I99.L99L99. | K99K99.L99L99. | | | | | | | |
| | | | | | L99L99.S99S99. | F99F99. | T99T99. | | | | | | | |
| | | | | | R99R99. | | | | | | | | | |
| 629 | 28 | VVGIVHE | GNCPR | TTVFSH | V99V99.V99V99. | G99G99.N99N99. | T99T99.T99T99. | 21 | 15 | 18 | 54 | Yes | Yes | Yes |
| | | | | | G99G99.I99I99. | C99C99.P99P99. | V99V99.F99F99. | | | | | | | |
| | | | | | V99V99.H96B00. | R99R99. | S99S99.H99H99. | | | | | | | |
| | | | | | E99E99. | | | | | | | | | |
| 634 | 28 | MADITSG | MDNVA | DLIRVSFGRLVLY | M92B00.A96B00. | M99M99.D96B00. | D99D99.L99L99. | 21 | 15 | 39 | 75 | Yes | Yes | Yes |
| | | | | | D99D99.I99I99. | N99N99.V99V99. | I99I99.R99R99. | | | | | | | |
| | | | | | T99T99.S99S99. | A99A99. | V99V99.S99S99. | | | | | | | |
| | | | | | G99G99. | | F99F99.G99G99. | | | | | | | |
| | | | | | | | R99R99.L96B00. | | | | | | | |
| | | | | | | | V99V99.L99L99. | | | | | | | |
| | | | | | | | Y99Y99. | | | | | | | |
| 657 | 28 | LFRRIVF | AGSVA | TTLLRTGSCS | L99L99.F99F99. | A99A99.G99G99. | T99T99.T99T99. | 21 | 15 | 30 | 66 | Yes | Yes | Yes |
| | | | | | R99R99.R99R99. | S99S99.V99V99. | L99L99.L99L99. | | | | | | | |
| | | | | | I99I99.V99V99. | A99A99. | R99R99.T99T99. | | | | | | | |
| | | | | | F99F99. | | G99G99.S99S99. | | | | | | | |
| | | | | | | | C99C99.S99S99. | | | | | | | |
| 779 | 28 | VV | EYGYR | LHGDPDDG | V99V99.V99V99. | E99E99.Y99Y99. | L99L99.H99H99. | 6 | 15 | 24 | 45 | Yes | Yes | Yes |
| | | | | | | G99G99.Y99Y99. | G99G99.D99D99. | | | | | | | |
| | | | | | | R99R99. | P99P99.D99D99. | | | | | | | |
| | | | | | | | D99D99.G99G99. | | | | | | | |
| 835 | 28 | SPCIFCC | FADKT | CVNHAIIWLS | S99S99.P99P99. | F99F99.A99A99. | C99C99.V99V99. | 21 | 15 | 30 | 66 | Yes | Yes | Yes |
| | | | | | C96B00.I99I99. | D99D99.K99K99. | N99N99.H99H99. | | | | | | | |
| | | | | | F99F99.C99C99. | T99T99. | A96B00.I99I99. | | | | | | | |
| | | | | | C99C99. | | I99I99.W99W99. | | | | | | | |
| | | | | | | | L99L99.S99S99. | | | | | | | |
| 106 | 27 | VVGTLSD | CFGTQ | TTRFKH | V99V99.V99V99. | C99C99.F99F99. | T99T99.T99T99. | 21 | 15 | 18 | 54 | Yes | Yes | Yes |
| | | | | | G99G99.T96B00. | G99G99.T99T99. | R99R99.F99F99. | | | | | | | |
| | | | | | L99L99.S99S99. | Q99Q99. | K96B00.H99H99. | | | | | | | |
| | | | | | D99D99. | | | | | | | | | |
| 179 | 27 | MFSSFWF | SGYYD | RFGFAG | M99M99.F99F99. | S99S99.G99G99. | R99R99.F99F99. | 21 | 15 | 18 | 54 | Yes | Yes | Yes |
| | | | | | S99S99.S99S99. | Y99Y99.Y99Y99. | G99G99.F99F99. | | | | | | | |
| | | | | | F99F99.W99W99. | D99D99. | A99A99.G99G99. | | | | | | | |
| | | | | | F99F99. | | | | | | | | | |
| 191 | 27 | SLHGVVF | KWLHE | MCLESTLVR | S99S99.L96B00. | K99K99.W99W99. | M96B00.C96B00. | 21 | 15 | 27 | 63 | Yes | Yes | Yes |
| | | | | | H99H99.G99G99. | L99L99.H99H99. | L96B00.E96B00. | | | | | | | |
| | | | | | V99V99.V99V99. | E99E99. | S96B00.T96B00. | | | | | | | |
| | | | | | F99F99. | | L96B00.V96B00. | | | | | | | |
| | | | | | | | R96B00. | | | | | | | |
| 272 | 27 | FMLRVVE | PMHNM | PYTPIDSVLDGIR | F99F99.M99M99. | P99P99.M99M99. | P99P99.Y99Y99. | 21 | 15 | 39 | 75 | Yes | Yes | Yes |
| | | | | | L99L99.R99R99. | H99H99.N99N99. | T99T99.P99P99. | | | | | | | |
| | | | | | V99V99.V99V99. | M99M99. | I99I99.D99D99. | | | | | | | |
| | | | | | E99E99. | | S99S99.V99V99. | | | | | | | |
| | | | | | | | L99L99.D99D99. | | | | | | | |
| | | | | | | | G99G99.I99I99. | | | | | | | |
| | | | | | | | R99R99. | | | | | | | |
| 45 | 27 | NERHCVF | PGHSG | VLFHPTFDPE | N92B00.E99E99. | P99P99.G99G99. | V99V99.L99L99. | 21 | 15 | 30 | 66 | Yes | Yes | Yes |
| | | | | | R99R99.H99H99. | H99H99.S99S99. | F99F99.H99H99. | | | | | | | |
| | | | | | C99C99.V99V99. | G99G99. | P99P99.T99T99. | | | | | | | |
| | | | | | F99F99. | | F96B00.D99D99. | | | | | | | |
| | | | | | | | P99P99.E99E99. | | | | | | | |
| 457 | 27 | PVKRCVY | ADLVG | VVMDTDIVGRDVI | P96B00.V96B00. | A99A99.D96B00. | V99V99.V99V99. | 21 | 15 | 39 | 75 | Yes | Yes | Yes |
| | | | | | K88B00.R96B00. | L96B00.V99V99. | M99M99.D99D99. | | | | | | | |
| | | | | | C96B00.V96B00. | G96B00. | T99T99.D99D99. | | | | | | | |
| | | | | | Y96B00. | | I99I99.V99V99. | | | | | | | |
| | | | | | | | G99G99.R99R99. | | | | | | | |
| | | | | | | | D99D99.V99V99. | | | | | | | |
| | | | | | | | I99I99. | | | | | | | |
| 63 | 27 | RIGCLVFE | AGNLI | VAYVTALSIC | R99R99.I99I99. | A99A99.G99G99. | V77B00.A77B00. | 21 | 15 | 20 | 56 | Yes | Yes | Yes |
| | | | | | G99G99.C99C99. | N99N99.L99L99. | Y77B00.V77B00. | | | | | | | |
| | | | | | L99L99.V99V99. | I99I99. | T77B00.A77B00. | | | | | | | |
| | | | | | F99F99. | | L77B00.S77B00. | | | | | | | |
| | | | | | | | I77B00.C77B00. | | | | | | | |
| 201 | 26 | R | PHNLG | LQFNPHYPLC | B00 | P96B00.H96B00. | L99L99.Q99Q99. | 0 | 15 | 30 | 45 | Yes | Yes | Yes |
| | | | | | | N96B00.L96B00. | F99F99.N99N99. | | | | | | | |
| | | | | | | G96B00. | P99P99.H99H99. | | | | | | | |
| | | | | | | | Y99Y99.P99P99. | | | | | | | |
| | | | | | | | L99L99.C99C99. | | | | | | | |
| 217 | 26 | CSFKYFI | DGSSI | FPTLHDSTAF | C99C99.S99S99. | D99D99.G96B00. | F99F99.P99P99. | 21 | 15 | 30 | 66 | Yes | Yes | Yes |
| | | | | | F99F99.K99K99. | S99S99.S99S99. | T99T99.L99L99. | | | | | | | |
| | | | | | Y99Y99.F99F99. | I99I99. | H99H99.D99D99. | | | | | | | |
| | | | | | I96B00. | | S99S99.T99T99. | | | | | | | |
| | | | | | | | A99A99.F99F99. | | | | | | | |
| 302 | 26 | PEAVLIA | FYPSG | NAVSSRGRWYGCI | P99P99.E99E99. | F96B00.Y99Y99. | B00 | 21 | 15 | 0 | 36 | Yes | Yes | Yes |
| | | | | | A96B00.V99V99. | P99P99.S99S99. | | | | | | | | |
| | | | | | L99L99.I96B00. | G99G99. | | | | | | | | |
| | | | | | A99A99. | | | | | | | | | |
| 584 | 26 | RIVQCVF | AGAVT | SRLTHQMYVL | R99R99.I99I99. | A99A99.G99G99. | S99S99.R99R99. | 21 | 15 | 30 | 66 | Yes | Yes | Yes |
| | | | | | V99V99.Q99Q99. | A99A99.V99V99. | L99L99.T99T99. | | | | | | | |
| | | | | | C99C99.V99V99. | T99T99. | H99H99.Q99Q99. | | | | | | | |
| | | | | | F99F99. | | M99M99.Y99Y99. | | | | | | | |
| | | | | | | | V99V99.L99L99. | | | | | | | |
| 618 | 26 | LVGFLGH | CNNAV | PYYDCLLGVL | L99L99.V96B00. | C99C99.N99N99. | P99P99.Y99Y99. | 21 | 15 | 30 | 66 | Yes | Yes | Yes |
| | | | | | G99G99.F99F99. | N99N99.A99A99. | Y99Y99.D99D99. | | | | | | | |
| | | | | | L99L99.G99G99. | V99V99. | C99C99.L99L99. | | | | | | | |
| | | | | | H99H99. | | L99L99.G99G99. | | | | | | | |
| | | | | | | | V99V99.L99L99. | | | | | | | |
| 687 | 26 | VTAHCFA | LDSRC | EVWRYVSVA | V99V99.T99T99. | L99L99.D99D99. | E57B00.V57B00. | 21 | 15 | 19 | 55 | Yes | Yes | Yes |
| | | | | | A99A99.H99H99. | S99S99.R99R99. | W57B00.R57B00. | | | | | | | |
| | | | | | C99C99.F99F99. | C99C99. | Y53B00.V57B00. | | | | | | | |
| | | | | | A99A99. | | S57B00.V88B00. | | | | | | | |
| | | | | | | | A57B00. | | | | | | | |
| 413 | 25 | VMFRACF | CEGFR | NDKHSYYSW | V99V99.M99M99. | C99C99.E99E99. | L99L99.N99N99. | 21 | 15 | 30 | 66 | Yes | Yes | Yes |
| | | | | | F99F99.R99R99. | G99G99.F99F99. | D99D99.K99K99. | | | | | | | |
| | | | | | A99A99.C99C99. | R99R99. | H99H99.S99S99. | | | | | | | |
| | | | | | F99F99. | | Y99Y99.Y99Y99. | | | | | | | |
| | | | | | | | S99S99.W99W99. | | | | | | | |
| 449 | 25 | R | PHNLG | LATLHYGGFSGAG | R99R99. | P99P99.H99H99. | L99L99.A99A99. | 3 | 15 | 39 | 57 | Yes | Yes | Yes |
| | | | | | | N99N99.L99L99. | T99T99.L99L99. | | | | | | | |
| | | | | | | G99G99. | H99H99.Y99Y99. | | | | | | | |
| | | | | | | | G99G99.G99G99. | | | | | | | |
| | | | | | | | F99F99.S99S99. | | | | | | | |
| | | | | | | | G99G99.A99A99. | | | | | | | |
| | | | | | | | G99G99. | | | | | | | |
| 608 | 25 | FLIRDMI | GRCSV | ITQHPDASFLLAD | F99F99.L99L99. | G99G99.R99R99. | I99I99.T99T99. | 21 | 15 | 39 | 75 | Yes | Yes | Yes |
| | | | | | I99I99.R99R99. | C99C99.S99S99. | Q99Q99.H99H99. | | | | | | | |
| | | | | | D99D99.M99M99. | V99V99. | P99P99.D96B00. | | | | | | | |
| | | | | | I99I99. | | A99A99.S99S99. | | | | | | | |
| | | | | | | | F99F99.L99L99. | | | | | | | |
| | | | | | | | L99L99.A99A99. | | | | | | | |
| | | | | | | | D99D99. | | | | | | | |
| 613 | 25 | VRPVPYQ | YCRCV | LACITTSQSW | V99V99.R99R99. | Y99Y99.C99C99. | L99L99.A99A99. | 21 | 15 | 30 | 66 | Yes | Yes | Yes |
| | | | | | P99P99.V99V99. | R99R99.C99C99. | C96B00.I99I99. | | | | | | | |
| | | | | | P99P99.Y99Y99. | V99V99. | T99T99.T99T99. | | | | | | | |
| | | | | | Q99Q99. | | S99S99.Q99Q99. | | | | | | | |
| | | | | | | | S99S99.W99W99. | | | | | | | |
| 195 | 24 | RMNGVCF | ICGAD | MICRATVEVV | R99R99.M99M99. | I99I99.C95B00. | M58H41.I58L41. | 21 | 15 | 21 | 57 | Yes | Yes | Yes |
| | | | | | N99N99.G99G99. | G99G99.A99A99. | C58Y41.R58H41. | | | | | | | |
| | | | | | V99V99.C99C99. | D99D99. | A58R41.T58H41. | | | | | | | |
| | | | | | F99F99. | | V58L37.E58G41. | | | | | | | |
| | | | | | | | V99V99.V58R41. | | | | | | | |
| 300 | 24 | GRTLV | LGYGN | HMCSSFNVS | G99G99.R99R99. | L99L99.G99G99. | B00 | 15 | 15 | 0 | 30 | Yes | Yes | Yes |
| | | | | | T99T99.L99L99. | Y99Y99.G99G99. | | | | | | | | |
| | | | | | V99V99. | N99N99. | | | | | | | | |
| 362 | 24 | IKVYYLV | NVS | MYGSSLSCY | I95B00.K99K99. | N99N99.V99V99. | B00 | 21 | 9 | 0 | 30 | Yes | Yes | Yes |
| | | | | | V99V99.Y99Y99. | S99S99. | | | | | | | | |
| | | | | | Y99Y99.L99L99. | | | | | | | | | |
| | | | | | V99V99. | | | | | | | | | |
| 434 | 24 | GMHSVVF | LARIQ | ARLCWDGVA | G99G99.M99M99. | L99L99.A99A99. | A99A99.R99R99. | 21 | 15 | 27 | 63 | Yes | Yes | Yes |
| | | | | | H99H99.S99S99. | R99R99.I99I99. | L99L99.C99C99. | | | | | | | |
| | | | | | V99V99.V99V99. | Q99Q99. | W99W99.D99D99. | | | | | | | |
| | | | | | F99F99. | | G99G99.V99V99. | | | | | | | |
| | | | | | | | A99A99. | | | | | | | |
| 451 | 24 | CQSLPCA | MLYFM | ERPHHSIQF | C99C99.Q95B00. | M99M99.L99L99. | E99E99.R99R99. | 21 | 15 | 27 | 63 | Yes | Yes | Yes |
| | | | | | S99S99.L99L99. | Y99Y99.F99F99. | P99P99.H95B00. | | | | | | | |
| | | | | | P99P99.C99C99. | M99M99. | H99H99.S99S99. | | | | | | | |
| | | | | | A99A99. | | I99I99.Q99Q99. | | | | | | | |
| | | | | | | | F99F99. | | | | | | | |
| 572 | 24 | LCVCRSS | NVLSF | YLIETNEGAVVRC | L99L99.C99C99. | N99N99.V99V99. | Y99Y99.L99L99. | 21 | 15 | 39 | 75 | Yes | Yes | Yes |
| | | | | | V95B00.C99C99. | L99L99.S99S99. | I99I99.E99E99. | | | | | | | |
| | | | | | R99R99.S99S99. | F99F99. | T99T99.N99N99. | | | | | | | |
| | | | | | S99S99. | | E99E99.G99G99. | | | | | | | |
| | | | | | | | A99A99.V99V99. | | | | | | | |
| | | | | | | | V99V99.R99R99. | | | | | | | |
| | | | | | | | C99C99. | | | | | | | |
| 582 | 24 | VPMDVLS | IHSVL | VDTSPVREMMRVK | V99V99.P99P99. | I99I99.H99H99. | V99V99.D99D99. | 21 | 15 | 39 | 75 | Yes | Yes | Yes |
| | | | | | M99M99.D99D99. | S99S99.V99V99. | T99T99.S99S99. | | | | | | | |
| | | | | | V99V99.L99L99. | L99L99. | P99P99.V99V99. | | | | | | | |
| | | | | | S99S99. | | R99R99.E99E99. | | | | | | | |
| | | | | | | | M99M99.M99M99. | | | | | | | |
| | | | | | | | R99R99.V99V99. | | | | | | | |
| | | | | | | | K99K99. | | | | | | | |
| 125 | 23 | IRAGCLI | TDD | YIYVLTCSRGTVR | I95B00.R99R99. | T99T99.D99D99. | B00 | 21 | 9 | 0 | 30 | Yes | Yes | Yes |
| | | | | | A99A99.G99G99. | D99D99. | | | | | | | | |
| | | | | | C99C99.L99L99. | | | | | | | | | |
| | | | | | I99I99. | | | | | | | | | |
| 144 | 23 | FVGLLLS | LYPYC | STHHMGFST | F95B00.V95B00. | L99L99.Y99Y99. | S99S99.T95B00. | 21 | 15 | 27 | 63 | Yes | Yes | Yes |
| | | | | | G95B00.L95B00. | P99P99.Y99Y99. | H99H99.H99H99. | | | | | | | |
| | | | | | L95B00.L91B00. | C99C99. | M99M99.G95B00. | | | | | | | |
| | | | | | S95B00. | | F99F99.S99S99. | | | | | | | |
| | | | | | | | T99T99. | | | | | | | |
| 182 | 23 | YVVGMIY | RASTS | MRFVGGSVR | Y95B00.V99V99. | R99R99.A99A99. | M99M99.R99R99. | 21 | 15 | 27 | 63 | Yes | Yes | Yes |
| | | | | | V99V99.G99G99. | S99S99.T99T99. | F99F99.V99V99. | | | | | | | |
| | | | | | M99M99.I99I99. | S99S99. | G99G99.G99G99. | | | | | | | |
| | | | | | Y95B00. | | S99S99.V99V99. | | | | | | | |
| | | | | | | | R99R99. | | | | | | | |
| 253 | 23 | WSSQ | YPGYY | LSCTQRCVRW | W99W99.S99S99. | Y99Y99.P99P99. | L99L99.S99S99. | 12 | 15 | 30 | 57 | Yes | Yes | Yes |
| | | | | | S99S99.Q99Q99. | G99G99.Y99Y99. | C99C99.T99T99. | | | | | | | |
| | | | | | | Y99Y99. | Q99Q99.R95B00. | | | | | | | |
| | | | | | | | C99C99.V95B00. | | | | | | | |
| | | | | | | | R99R99.W99W99. | | | | | | | |
| 26 | 23 | CLAGVVF | PVVC | AVLDRVVR | C99C99.L99L99. | P99P99.V99V99. | A99A99.V99V99. | 21 | 12 | 24 | 57 | Yes | Yes | Yes |
| | | | | | A99A99.G99G99. | V99V99.C99C99. | L99L99.D99D99. | | | | | | | |
| | | | | | V99V99.V99V99. | | R99R99.V99V99. | | | | | | | |
| | | | | | F99F99. | | V99V99.R99R99. | | | | | | | |
| 318 | 23 | FGDLIGC | FLLHS | FCSADR | F99F99.G95B00. | F99F99.L99L99. | F99F99.C99C99. | 21 | 15 | 18 | 54 | Yes | Yes | Yes |
| | | | | | D99D99.L99L99. | L99L99.H99H99. | S99S99.A99A99. | | | | | | | |
| | | | | | I99I99.G99G99. | S99S99. | D99D99.R99R99. | | | | | | | |
| | | | | | C99C99. | | | | | | | | | |
| 350 | 23 | ICAGFCR | VGHIR | QHVSYYNCLLSWS | I91B00.C99C99. | V99V99.G99G99. | Q99Q99.H99H99. | 21 | 15 | 39 | 75 | Yes | Yes | Yes |
| | | | | | A99A99.G99G99. | H99H99.I99I99. | V99V99.S99S99. | | | | | | | |
| | | | | | F99F99.C99C99. | R99R99. | Y99Y99.Y99Y99. | | | | | | | |
| | | | | | R99R99. | | N99N99.C99C99. | | | | | | | |
| | | | | | | | L99L99.L99L99. | | | | | | | |
| | | | | | | | S99S99.W95B00. | | | | | | | |
| | | | | | | | S99S99. | | | | | | | |
| 394 | 23 | RTNGMVY | RSYYG | ARVDASGVVS | R99R99.T99T99. | R99R99.S99S99. | A99A99.R95B00. | 21 | 15 | 30 | 66 | Yes | Yes | Yes |
| | | | | | N99N99.G99G99. | Y99Y99.Y99Y99. | V95B00.D95B00. | | | | | | | |
| | | | | | M99M99.V99V99. | G99G99. | A95B00.S95B00. | | | | | | | |
| | | | | | Y99Y99. | | G99G99.V95B00. | | | | | | | |
| | | | | | | | V99V99.S95B00. | | | | | | | |
| 41 | 23 | SHCSFMY | RLLDD | RMLGMAKGSIRWS | S95B00.H95B00. | R99R99.L99L99. | R99R99.M99M99. | 21 | 15 | 39 | 75 | Yes | Yes | Yes |
| | | | | | C95B00.S95B00. | L99L99.D99D99. | L99L99.G99G99. | | | | | | | |
| | | | | | F95B00.M95B00. | D99D99. | M99M99.A99A99. | | | | | | | |
| | | | | | Y95B00. | | K99K99.G99G99. | | | | | | | |
| | | | | | | | S99S99.I99I99. | | | | | | | |
| | | | | | | | R95B00.W99W99. | | | | | | | |
| | | | | | | | S99S99. | | | | | | | |
| 452 | 23 | IDVLLTR | CWVLE | TWNAAHFWDC | I99I99.D99D99. | C99C99.W99W99. | T99T99.W99W99. | 21 | 15 | 30 | 66 | Yes | Yes | Yes |
| | | | | | V99V99.L99L99. | V99V99.L99L99. | N99N99.A99A99. | | | | | | | |
| | | | | | L99L99.T99T99. | E99E99. | A95B00.H99H99. | | | | | | | |
| | | | | | R99R99. | | F99F99.W99W99. | | | | | | | |
| | | | | | | | D99D99.C99C99. | | | | | | | |
| 463 | 23 | VLSLLLA | RHACS | FTRFSI | V99V99.L99L99. | R99R99.H99H99. | F99F99.T99T99. | 21 | 15 | 18 | 54 | Yes | Yes | Yes |
| | | | | | S95B00.L99L99. | A99A99.C99C99. | R99R99.F99F99. | | | | | | | |
| | | | | | L99L99.L99L99. | S99S99. | S99S99.H99H99. | | | | | | | |
| | | | | | A95B00. | | | | | | | | | |
| 480 | 23 | LDS | ASGNA | TVVYLF | L99L99.D99D99. | A99A99.S99S99. | T99T99.V99V99. | 9 | 15 | 18 | 42 | Yes | Yes | Yes |
| | | | | | S95B00. | G99G99.N99N99. | V99V99.Y99Y99. | | | | | | | |
| | | | | | | A99A99. | L99L99.F99F99. | | | | | | | |
| 514 | 23 | SVFQIHW | HICGS | LLQNHGNMVY | S99S99.V99V99. | H99H99.I99I99. | L95B00.L99L99. | 21 | 15 | 30 | 66 | Yes | Yes | Yes |
| | | | | | F99F99.Q99Q99. | C99C99.G99G99. | Q99Q99.N99N99. | | | | | | | |
| | | | | | I99I99.H99H99. | S99S99. | H99H99.G99G99. | | | | | | | |
| | | | | | W99W99. | | N99N99.M99M99. | | | | | | | |
| | | | | | | | V99V99.Y99Y99. | | | | | | | |
| 537 | 23 | LFKNVVF | NNGLP | ALLAPARCVL | L99L99.F99F99. | N99N99.N99N99. | A99A99.L73B00. | 21 | 15 | 22 | 58 | Yes | Yes | Yes |
| | | | | | K99K99.N99N99. | G99G99.L99L99. | L73B00.A73B00. | | | | | | | |
| | | | | | V99V99.V99V99. | P99P99. | P73B00.A73B00. | | | | | | | |
| | | | | | F99F99. | | R73B00.C73B00. | | | | | | | |
| | | | | | | | V99V99.L73B00. | | | | | | | |
| 712 | 23 | LVRGSVF | YCDHD | VHRDHDTSLV | L95B00.V99V99. | Y99Y99.C99C99. | V99V99.H99H99. | 21 | 15 | 30 | 66 | Yes | Yes | Yes |
| | | | | | R99R99.G99G99. | D99D99.H99H99. | R99R99.D99D99. | | | | | | | |
| | | | | | S99S99.V99V99. | D99D99. | H99H99.D95B00. | | | | | | | |
| | | | | | F99F99. | | T99T99.S99S99. | | | | | | | |
| | | | | | | | L99L99.V99V99. | | | | | | | |
| 115 | 22 | LCSRVVI | TSLSG | HRLCDTGSVR | L95B00.C90B00. | T99T99.S99S99. | H99H99.R99R99. | 21 | 15 | 30 | 66 | Yes | Yes | Yes |
| | | | | | S95B00.R95B00. | L99L99.S99S99. | L99L99.C99C99. | | | | | | | |
| | | | | | V95B00.V95B00. | G99G99. | D99D99.T99T99. | | | | | | | |
| | | | | | I95B00. | | G99G99.S99S99. | | | | | | | |
| | | | | | | | V99V99.R95B00. | | | | | | | |
| 115 | 22 | KYIAYTP | LMRGI | AWALLCEASES | K95B00.Y99Y99. | L95B00.M99M99. | A99A99.W99W99. | 21 | 15 | 33 | 69 | Yes | Yes | Yes |
| | | | | | I90B00.A99A99. | R99R99.G99G99. | A99A99.L99L99. | | | | | | | |
| | | | | | Y99Y99.T99T99. | I99I99. | L99L99.C99C99. | | | | | | | |
| | | | | | P99P99. | | E99E99.A99A99. | | | | | | | |
| | | | | | | | S95B00.E99E99. | | | | | | | |
| | | | | | | | S99S99. | | | | | | | |
| 206 | 22 | IVGLLVC | FSPTA | CTRFRA | I99I99.V99V99. | F99F99.S99S99. | C99C99.T99T99. | 21 | 15 | 18 | 54 | Yes | Yes | Yes |
| | | | | | G99G99.L99L99. | P99P99.T99T99. | R99R99.F99F99. | | | | | | | |
| | | | | | L99L99.V99V99. | A99A99. | R99R99.A99A99. | | | | | | | |
| | | | | | C99C99. | | | | | | | | | |
| 249 | 22 | KVSDVYV | SSRHA | QTMSYPGVWLIIQ | K95B00.V99V99. | S99S99.S99S99. | Q99Q99.T99T99. | 21 | 15 | 39 | 75 | Yes | Yes | Yes |
| | | | | | S99S99.D99D99. | R99R99.H99H99. | M99M99.S99S99. | | | | | | | |
| | | | | | V99V99.Y99Y99. | A95B00. | Y99Y99.P99P99. | | | | | | | |
| | | | | | V99V99. | | G99G99.V99V99. | | | | | | | |
| | | | | | | | W99W99.L99L99. | | | | | | | |
| | | | | | | | I99I99.I99I99. | | | | | | | |
| | | | | | | | Q99Q99. | | | | | | | |
| 252 | 22 | HHYHLFA | SSGG | TACATTPGEVDVH | H99H99.H95B00. | S99S99.S99S99. | T95B00.A99A99. | 21 | 12 | 39 | 72 | Yes | Yes | Yes |
| | | | | | Y99Y99.H99H99. | G99G99.G99G99. | C95B00.A95B00. | | | | | | | |
| | | | | | L99L99.F99F99. | | T95B00.T95B00. | | | | | | | |
| | | | | | A99A99. | | P95B00.G95B00. | | | | | | | |
| | | | | | | | E95B00.V95B00. | | | | | | | |
| | | | | | | | D95B00.V95B00. | | | | | | | |
| | | | | | | | H95B00. | | | | | | | |
| 295 | 22 | LGRLVL | EFGGQ | ITISGARSYMSSH | L99L99.G99G99. | E95B00.F99F99. | I99I99.T99T99. | 18 | 15 | 39 | 72 | Yes | Yes | Yes |
| | | | | | R99R99.L99L99. | G99G99.G99G99. | I99I99.S99S99. | | | | | | | |
| | | | | | V99V99.L99L99. | Q99Q99. | G99G99.A99A99. | | | | | | | |
| | | | | | | | R99R99.S99S99. | | | | | | | |
| | | | | | | | Y99Y99.M99M99. | | | | | | | |
| | | | | | | | S99S99.S99S99. | | | | | | | |
| | | | | | | | H95B00. | | | | | | | |
| 332 | 22 | RFSSAVY | RDNKG | MYLKSRSEIG | R95B00.F95B00. | R99R99.D99D99. | M99M99.Y99Y99. | 21 | 15 | 30 | 66 | Yes | Yes | Yes |
| | | | | | S95B00.S95B00. | N99N99.K99K99. | L99L99.K99K99. | | | | | | | |
| | | | | | A95B00.V99V99. | G99G99. | S99S99.R99R99. | | | | | | | |
| | | | | | Y95B00. | | S99S99.E99E99. | | | | | | | |
| | | | | | | | I99I99.G99G99. | | | | | | | |
| 575 | 22 | SARKCVQ | ASGEV | LIANPVVR | S95B00.A95B00. | A99A99.S99S99. | L99L99.I99I99. | 21 | 15 | 24 | 60 | Yes | Yes | Yes |
| | | | | | R95B00.K95B00. | G99G99.E99E99. | A99A99.N99N99. | | | | | | | |
| | | | | | C95B00.V95B00. | V99V99. | P99P99.V99V99. | | | | | | | |
| | | | | | Q95B00. | | V99V99.R95B00. | | | | | | | |
| 58 | 22 | VDIRNMC | GWLCAA | RPTHALVSVY | V99V99.D95B00. | G99G99.W99W99. | R90B00.P95B00. | 21 | 15 | 30 | 66 | Yes | Yes | Yes |
| | | | | | I99I99.R99R99. | L99L99.C99C99. | T95B00.H95B00. | | | | | | | |
| | | | | | N95B00.M99M99. | A99A99. | A95B00.L95B00. | | | | | | | |
| | | | | | C99C99. | | V95B00.S99S99. | | | | | | | |
| | | | | | | | V95B00.Y95B00. | | | | | | | |
| 100 | 21 | IRNQLLF | AGIGA | FYFSTHFNVI | I90B00.R95B00. | A95B00.G95B00. | F99F99.Y99Y99. | 21 | 15 | 30 | 66 | Yes | Yes | Yes |
| | | | | | N90B00.Q95B00. | I95B00.G95B00. | F99F99.S99S99. | | | | | | | |
| | | | | | L95B00.L95B00. | A95B00. | T99T99.H99H99. | | | | | | | |
| | | | | | F95B00. | | F99F99.N99N99. | | | | | | | |
| | | | | | | | V99V99.I99I99. | | | | | | | |
| 162 | 21 | HCDGGFM | QYGYR | DTNHWPNSCS | H99H99.C99C99. | Q99Q99.Y99Y99. | D47L42.T52K47. | 21 | 15 | 13 | 49 | Yes | Yes | Yes |
| | | | | | D99D99.G99G99. | G99G99.Y99Y99. | N47Y42.H52T47. | | | | | | | |
| | | | | | G99G99.F99F99. | R99R99. | W47142.P47L42. | | | | | | | |
| | | | | | M95B00. | | N57P42.S47L42. | | | | | | | |
| | | | | | | | C47Y42.S47W42. | | | | | | | |
| 207 | 21 | NLVRLMG | IYPAA | AILRPAAVCS | N95B00.L99L99. | I99I99.Y99Y99. | A99A99.I99I99. | 21 | 15 | 30 | 66 | Yes | Yes | Yes |
| | | | | | V99V99.R99R99. | P99P99.A99A99. | L99L99.R99R99. | | | | | | | |
| | | | | | L99L99.M99M99. | A99A99. | P99P99.A99A99. | | | | | | | |
| | | | | | G99G99. | | A99A99.V99V99. | | | | | | | |
| | | | | | | | C99C99.S99S99. | | | | | | | |
| 351 | 21 | MCYRCFN | YRWTV | FPSDCMDCGGLVF | M99M99.C99C99. | Y99Y99.R99R99. | F99F99.P99P99. | 21 | 15 | 39 | 75 | Yes | Yes | Yes |
| | | | | | Y99Y99.R99R99. | W99W99.T95B00. | S99S99.D99D99. | | | | | | | |
| | | | | | C99C99.F99F99. | V99V99. | C99C99.M99M99. | | | | | | | |
| | | | | | N99N99. | | D99D99.C99C99. | | | | | | | |
| | | | | | | | G99G99.G99G99. | | | | | | | |
| | | | | | | | L99L99.V99V99. | | | | | | | |
| | | | | | | | F99F99. | | | | | | | |
| 516 | 21 | TRFWSSL | TNGCC | HIITKSKFGTCVT | T90B00.R95B00. | T95B00.N95B00. | H99H99.I99I99. | 21 | 15 | 39 | 75 | Yes | Yes | Yes |
| | | | | | F95B00.W95B00. | G95B00.C95B00. | I99I99.T99T99. | | | | | | | |
| | | | | | S95B00.S95B00. | C95B00. | K99K99.S95B00. | | | | | | | |
| | | | | | L95B00. | | K99K99.F99F99. | | | | | | | |
| | | | | | | | G99G99.T99T99. | | | | | | | |
| | | | | | | | C99C99.V99V99. | | | | | | | |
| | | | | | | | T99T99. | | | | | | | |
| 550 | 21 | ASLPWVL | RSVMS | IYVCTGQVD | A95B00.S95B00. | R99R99.S99S99. | I99I99.Y95B00. | 21 | 15 | 27 | 63 | Yes | Yes | Yes |
| | | | | | L99L99.P95B00. | V99V99.M99M99. | V99V99.C95B00. | | | | | | | |
| | | | | | W95B00.V95B00. | S99S99. | T99T99.G99G99. | | | | | | | |
| | | | | | L95B00. | | Q99Q99.V99V99. | | | | | | | |
| | | | | | | | D99D99. | | | | | | | |
| 686 | 21 | SFVDRIF | PYDAG | FRPDHSGSCV | S95B00.F99F99. | P99P99.Y99Y99. | F99F99.R99R99. | 21 | 15 | 30 | 66 | Yes | Yes | Yes |
| | | | | | V99V99.D99D99. | D99D99.A99A99. | P99P99.D99D99. | | | | | | | |
| | | | | | R99R99.I99I99. | G99G99. | H99H99.S99S99. | | | | | | | |
| | | | | | F99F99. | | G99G99.S99S99. | | | | | | | |
| | | | | | | | C99C99.V99V99. | | | | | | | |
| 220 | 20 | IVILVCQ | FRGVT | NDPSAV | I99I99.V99V99. | F99F99.R99R99. | N99N99.D99D99. | 21 | 15 | 18 | 54 | Yes | Yes | Yes |
| | | | | | I99I99.L99L99. | G99G99.V99V99. | P99P99.S99S99. | | | | | | | |
| | | | | | V99V99.C99C99. | T99T99. | A90B00.V99V99. | | | | | | | |
| | | | | | Q99Q99. | | | | | | | | | |
| 239 | 20 | GVQCPVL | EFGWS | VIINVTDLMGPTI | G99G99.V99V99. | E95B00.F99F99. | B00 | 21 | 15 | 0 | 36 | Yes | Yes | Yes |
| | | | | | Q99Q99.C99C99. | G99G99.W99W99. | | | | | | | | |
| | | | | | P99P99.V99V99. | S99S99. | | | | | | | | |
| | | | | | L99L99. | | | | | | | | | |
| 353 | 20 | YGFHGSV | PLRHV | TITEPF | Y99Y99.G99G99. | P99P99.L99L99. | T99T99.I99I99. | 21 | 15 | 18 | 54 | Yes | Yes | Yes |
| | | | | | F99F99.H99H99. | R99R99.H99H99. | T99T99.E99E99. | | | | | | | |
| | | | | | G99G99.S99S99. | V99V99. | P95B00.F99F99. | | | | | | | |
| | | | | | V99V99. | | | | | | | | | |
| 365 | 20 | GRYVGVV | LRTDR | TCYIDGERNGRWF | G95B00.R95B00. | L99L99.R99R99. | T99T99.C99C99. | 21 | 15 | 39 | 75 | Yes | Yes | Yes |
| | | | | | Y95B00.V95B00. | T99T99.D99D99. | Y99Y99.I99I99. | | | | | | | |
| | | | | | G95B00.V99V99. | R99R99. | D99D99.G99G99. | | | | | | | |
| | | | | | V95B00. | | E99E99.R99R99. | | | | | | | |
| | | | | | | | N99N99.G99G99. | | | | | | | |
| | | | | | | | R99R99.W95B00. | | | | | | | |
| | | | | | | | F99F99. | | | | | | | |
| 510 | 20 | LIVSSGL | CVGLF | FTFCSPTSRI | L99L99.I99I99. | C99C99.V99V99. | F99F99.T99T99. | 21 | 15 | 30 | 66 | Yes | Yes | Yes |
| | | | | | V99V99.S99S99. | G99G99.L99L99. | F99F99.C99C99. | | | | | | | |
| | | | | | S99S99.G99G99. | F99F99. | S99S99.P99P99. | | | | | | | |
| | | | | | L99L99. | | T95B00.S99S99. | | | | | | | |
| | | | | | | | R99R99.I99I99. | | | | | | | |
| 532 | 20 | LLALTVF | NCGSV | ANLRPNDTVY | L95B00.L95B00. | N95B00.C95B00. | A99A99.N99N99. | 21 | 15 | 30 | 66 | Yes | Yes | Yes |
| | | | | | A90B00.L95B00. | G95B00.S95B00. | L99L99.R99R99. | | | | | | | |
| | | | | | T95B00.V99V99. | V95B00. | P99P99.N99N99. | | | | | | | |
| | | | | | F95B00. | | D99D99.T99T99. | | | | | | | |
| | | | | | | | V99V99.Y99Y99. | | | | | | | |
| 535 | 20 | LKRLTI | LVSSS | YAYVSIGVMR | L95B00.K80B00. | L95B00.V95B00. | Y99Y99.A99A99. | 17 | 15 | 30 | 62 | Yes | Yes | Yes |
| | | | | | R99R99.L95B00. | S95B00.S95B00. | Y99Y99.V99V99. | | | | | | | |
| | | | | | T95B00.I95B00. | S95B00. | S99S99.I99I99. | | | | | | | |
| | | | | | | | G99G99.V99V99. | | | | | | | |
| | | | | | | | M99M99.R99R99. | | | | | | | |
| 661 | 20 | MIGVVY | PDSIQ | FTFAPR | M99M99.I99I99. | P99P99.D99D99. | F95B00.T95B00. | 18 | 15 | 18 | 51 | Yes | Yes | Yes |
| | | | | | G99G99.V99V99. | S99S99.I99I99. | F95B00.A95B00. | | | | | | | |
| | | | | | V99V99.Y99Y99. | Q99Q99. | P90B00.R95B00. | | | | | | | |
| 773 | 20 | CYRQRVF | AAWPM | ATFSDTNGVI | C99C99.Y99Y99. | A99A99.A99A99. | A99A99.T99T99. | 21 | 15 | 30 | 66 | Yes | Yes | Yes |
| | | | | | R99R99.Q99Q99. | W99W99.P99P99. | F95B00.S99S99. | | | | | | | |
| | | | | | R99R99.V99V99. | M95B00. | D99D99.T99T99. | | | | | | | |
| | | | | | F99F99. | | N99N99.G99G99. | | | | | | | |
| | | | | | | | V99V99.I99I99. | | | | | | | |
| 11 | 19 | AGDRVLL | TDVPT | LTVRVQYLC | A99A99.G99G99. | T99T99.D99D99. | L99L99.T99T99. | 21 | 15 | 27 | 63 | Yes | Yes | Yes |
| | | | | | D99D99.R99R99. | V99V99.P99P99. | V99V99.R99R99. | | | | | | | |
| | | | | | V99V99.L99L99. | T99T99. | V99V99.Q99Q99. | | | | | | | |
| | | | | | L99L99. | | Y99Y99.L99L99. | | | | | | | |
| | | | | | | | C99C99. | | | | | | | |
| 158 | 19 | DTMDRCF | PFSFR | NDLANDIQFS | D99D99.T99T99. | P99P99.F99F99. | N73B00.D73B00. | 21 | 15 | 22 | 58 | Yes | Yes | Yes |
| | | | | | M99M99.D99D99. | S99S99.F99F99. | L99L99.A73B00. | | | | | | | |
| | | | | | R99R99.C99C99. | R99R99. | N73B00.D84B00. | | | | | | | |
| | | | | | F99F99. | | I73B00.Q73B00. | | | | | | | |
| | | | | | | | F73B00.S73B00. | | | | | | | |
| 102 | 19 | SAGFRVF | ADGER | AFLWLGSVA | S99S99.A99A99. | A99A99.D99D99. | B00 | 21 | 15 | 0 | 36 | Yes | Yes | Yes |
| | | | | | G99G99.F99F99. | G99G99.E99E99. | | | | | | | | |
| | | | | | R99R99.V99V99. | R99R99. | | | | | | | | |
| | | | | | F99F99. | | | | | | | | | |
| 235 | 19 | ALSD | GHSFS | WRVRHADIWF | A99A99.L99L99. | G99G99.H99H99. | W99W99.R99R99. | 12 | 15 | 30 | 57 | Yes | Yes | Yes |
| | | | | | S99S99.D99D99. | S99S99.F99F99. | V99V99.R99R99. | | | | | | | |
| | | | | | | S99S99. | H99H99.A99A99. | | | | | | | |
| | | | | | | | D99D99.I99I99. | | | | | | | |
| | | | | | | | W99W99.F99F99. | | | | | | | |
| 236 | 19 | GFYGLL | TSVAR | YDLLAHGTVR | G99G99.F99F99. | T99T99.S99S99. | Y99Y99.D99D99. | 18 | 15 | 30 | 63 | Yes | Yes | Yes |
| | | | | | Y99Y99.G99G99. | V99V99.A99A99. | L99L99.L99L99. | | | | | | | |
| | | | | | L99L99.L99L99. | R99R99. | A99A99.H99H99. | | | | | | | |
| | | | | | | | G94B00.T99T99. | | | | | | | |
| | | | | | | | V99V99.R99R99. | | | | | | | |
| 264 | 19 | NTWLCFR | HFLNS | HFSTGDNIVL | N99N99.T94B00. | H99H99.F99F99. | H99H99.F99F99. | 21 | 15 | 30 | 66 | Yes | Yes | Yes |
| | | | | | W99W99.L99L99. | L99L99.N99N99. | S99S99.T99T99. | | | | | | | |
| | | | | | C99C99.F99F99. | S99S99. | G99G99.D99D99. | | | | | | | |
| | | | | | R99R99. | | N99N99.I99I99. | | | | | | | |
| | | | | | | | V99V99.L99L99. | | | | | | | |
| 359 | 19 | IVDMEFQ | LSHLR | YHCLGR | I99I99.V99V99. | L99L99.S99S99. | Y99Y99.H99H99. | 21 | 15 | 18 | 54 | Yes | Yes | Yes |
| | | | | | D99D99.M99M99. | H99H99.L99L99. | C99C99.L99L99. | | | | | | | |
| | | | | | E99E99.F99F99. | R99R99. | G99G99.R99R99. | | | | | | | |
| | | | | | Q99Q99. | | | | | | | | | |
| 441 | 19 | FMTHIVY | PGYSL | ICLEERMVCK | F99F99.M99M99. | P99P99.G99G99. | I99I99.C99C99. | 21 | 15 | 30 | 66 | Yes | Yes | Yes |
| | | | | | T99T99.H99H99. | Y99Y99.S99S99. | L99L99.E99E99. | | | | | | | |
| | | | | | I99I99.V99V99. | L99L99. | E99E99.R99R99. | | | | | | | |
| | | | | | Y99Y99. | | M99M99.V99V99. | | | | | | | |
| | | | | | | | C99C99.K99K99. | | | | | | | |
| 494 | 19 | SIQKVVY | TSGCD | ACFIHFRGVF | S99S99.I99I99. | T99T99.S99S99. | A73B00.C73B00. | 21 | 15 | 20 | 56 | Yes | Yes | Yes |
| | | | | | Q99Q99.K99K99. | G99G99.C99C99. | F73B00.I73B00. | | | | | | | |
| | | | | | V99V99.V99V99. | D99D99 | H73B00.F73B00. | | | | | | | |
| | | | | | Y99Y99. | | R73B00.G73B00. | | | | | | | |
| | | | | | | | V73B00.F73B00. | | | | | | | |
| 605 | 19 | CTNIYC | VFRRA | QTECHPLLQ | C99C99.T99T99. | V99V99.F99F99. | Q99Q99.T94B00. | 18 | 15 | 27 | 60 | Yes | Yes | Yes |
| | | | | | N99N99.I99I99. | R99R99.R94B00. | E99E99.C99C99. | | | | | | | |
| | | | | | Y99Y99.C99C99. | A99A99. | H99H99.P99P99. | | | | | | | |
| | | | | | | | L99L99.L99L99. | | | | | | | |
| | | | | | | | Q99Q99. | | | | | | | |
| 104 | 18 | MSVMCMS | GLDRY | DYATLVICGY | M94B00.S99S99. | G94B00.L99L99. | D99D99.Y99Y99. | 21 | 15 | 30 | 66 | Yes | Yes | Yes |
| | | | | | V99V99.M99M99. | D99D99.R99R99. | A99A99.T99T99. | | | | | | | |
| | | | | | C99C99.M94B00. | Y99Y99. | L99L99.V99V99. | | | | | | | |
| | | | | | S99S99. | | I99I99.C99C99. | | | | | | | |
| | | | | | | | G99G99.Y99Y99. | | | | | | | |
| 127 | 18 | QYLAVCW | RAAVT | HVLLSSSESY | Q99Q99.Y99Y99. | R99R99.A99A99. | H94B00.V94B00. | 21 | 15 | 30 | 66 | Yes | Yes | Yes |
| | | | | | L99L99.A99A99. | A99A99.V99V99. | L94B00.L94B00. | | | | | | | |
| | | | | | V99V99.C99C99. | T94B00. | S94B00.S94B00. | | | | | | | |
| | | | | | W99W99. | | S94B00.E94B00. | | | | | | | |
| | | | | | | | S94B00.Y94B00. | | | | | | | |
| 185 | 18 | WFC | NYPEH | HAIDSKGLRT | W99W99.F99F99. | N99N99.Y99Y99. | H99H99.A94B00. | 9 | 15 | 30 | 54 | Yes | Yes | Yes |
| | | | | | C99C99. | P99P99.E99E99. | I99I99.D99D99. | | | | | | | |
| | | | | | | H99H99. | S99S99.K99K99. | | | | | | | |
| | | | | | | | G99G99.L99L99. | | | | | | | |
| | | | | | | | R99R99.T99T99. | | | | | | | |
| 267 | 18 | LIRHFFV | MPGVV | VSWWED | L99L99.I99I99. | M99M99.P99P99. | V99V99.S99S99. | 21 | 15 | 18 | 54 | Yes | Yes | Yes |
| | | | | | R99R99.H99H99. | G99G99.V99V99. | W99W99.W99W99. | | | | | | | |
| | | | | | F99F99.F99F99. | V99V99. | E99E99.D99D99. | | | | | | | |
| | | | | | V99V99. | | | | | | | | | |
| 287 | 18 | CYGVVYS | IGGSR | LCHHDTFVVH | C99C99.Y94B00. | I99I99.G99G99. | L99L99.C94B00. | 21 | 15 | 30 | 66 | Yes | Yes | Yes |
| | | | | | G99G99.V99V99. | G99G99.S99S99. | H99H99.H99H99. | | | | | | | |
| | | | | | V99V99.Y99Y99. | R99R99. | D99D99.T99T99. | | | | | | | |
| | | | | | S99S99. | | F99F99.V99V99. | | | | | | | |
| | | | | | | | V94B00.H99H99. | | | | | | | |
| 316 | 18 | LRNCLW | HAPQD | ASYTLTGVVD | L99L99.R99R99. | H99H99.A99A99. | A99A99.S94B00. | 18 | 15 | 29 | 62 | Yes | Yes | Yes |
| | | | | | N99N99.C99C99. | P99P99.Q99Q99. | Y99Y99.T99T99. | | | | | | | |
| | | | | | L99L99.W99W99. | D99D99. | L99L99.T99T99. | | | | | | | |
| | | | | | | | G99G99.V99V99. | | | | | | | |
| | | | | | | | V99V99.D55N44. | | | | | | | |
| 335 | 18 | GYIRQNW | FYTIS | TWYCDLGFF | G99G99.Y94B00. | F99F99.Y99Y99. | T83B00.W83B00. | 21 | 15 | 27 | 63 | Yes | Yes | Yes |
| | | | | | I88B00.R99R99. | T99T99.I99I99. | Y99Y99.C83B00. | | | | | | | |
| | | | | | Q99Q99.N94B00. | S99S99. | D83B00.L83B00. | | | | | | | |
| | | | | | W99W99. | | G83B00.F83B00. | | | | | | | |
| | | | | | | | F83B00. | | | | | | | |
| 377 | 18 | TIRSCVF | NDVDG | AAFLTTVMGVI | T94B00.I99I99. | N99N99.D99D99. | B00 | 21 | 15 | 0 | 36 | Yes | Yes | Yes |
| | | | | | R99R99.S99S99. | V99V99.D99D99. | | | | | | | | |
| | | | | | C99C99.V99V99. | G99G99. | | | | | | | | |
| | | | | | F99F99. | | | | | | | | | |
| 399 | 18 | LIAPVVY | RAGRC | IAFRTHDCVL | L99L99.I99I99. | R99R99.A99A99. | I99I99.A99A99. | 21 | 15 | 30 | 66 | Yes | Yes | Yes |
| | | | | | A99A99.P99P99. | G99G99.R99R99. | F99F99.R99R99. | | | | | | | |
| | | | | | V99V99.V99V99. | C99C99. | T99T99.H99H99. | | | | | | | |
| | | | | | Y99Y99. | | D99D99.C99C99. | | | | | | | |
| | | | | | | | V99V99.L99L99. | | | | | | | |
| 445 | 18 | IQRRVA | GFTAP | RALAISHAVL | I99I99.Q99Q99. | G99G99.F99F99. | R99R99.A99A99. | 18 | 15 | 30 | 63 | Yes | Yes | Yes |
| | | | | | R99R99.R99R99. | T99T99.A99A99. | L99L99.A99A99. | | | | | | | |
| | | | | | V99V99.A99A99. | P99P99. | I99I99.S99S99. | | | | | | | |
| | | | | | | | H99H99.A99A99. | | | | | | | |
| | | | | | | | V99V99.L99L99. | | | | | | | |
| 50 | 18 | NIRRIVY | CDGTT | SHLTNFLLVH | N94B00.I99I99. | C99C99.D99D99. | S99S99.H99H99. | 21 | 15 | 30 | 66 | Yes | Yes | Yes |
| | | | | | R99R99.R99R99. | G99G99.T99T99. | L99L99.T99T99. | | | | | | | |
| | | | | | I99I99.V99V99. | T99T99. | N99N99.F99F99. | | | | | | | |
| | | | | | Y99Y99. | | L99L99.L99L99. | | | | | | | |
| | | | | | | | V99V99.H99H99. | | | | | | | |
| 533 | 18 | CCHVFG | FGG | HAWDTNVSVT | C99C99.C99C99. | F99F99.G99G99. | H99H99.A99A99. | 18 | 15 | 30 | 63 | Yes | Yes | Yes |
| | | | | | H99H99.V99V99. | D99D99.I99I99. | W99W99.D99D99. | | | | | | | |
| | | | | | F99F99.G99G99. | G99G99. | T94B00.N99N99. | | | | | | | |
| | | | | | | | V99V99.S99S99. | | | | | | | |
| | | | | | | | V99V99.T99T99. | | | | | | | |
| 594 | 18 | IPYAQRS | MLLKS | VDDYC | I99I99.P99P99. | M99M99.L99L99. | V99V99.D99D99. | 21 | 15 | 15 | 51 | Yes | Yes | Yes |
| | | | | | Y99Y99.A99A99. | L99L99.K99K99. | D99D99.Y99Y99. | | | | | | | |
| | | | | | Q99Q99.R99R99. | S99S99. | C99C99. | | | | | | | |
| | | | | | S94B00. | | | | | | | | | |
| 647 | 18 | SFLTVLS | GILCE | TVGSYHLDCLCVC | S99S99.F99F99. | G99G99.I99I99. | T99T99.V99V99. | 21 | 15 | 39 | 75 | Yes | Yes | Yes |
| | | | | | L99L99.T99T99. | L99L99.C99C99. | G99G99.S99S99. | | | | | | | |
| | | | | | V99V99.L99L99. | E99E99. | Y99Y99.H99H99. | | | | | | | |
| | | | | | S99S99. | | L99L99.D99D99. | | | | | | | |
| | | | | | | | C99C99.L99L99. | | | | | | | |
| | | | | | | | C99C99.V99V99. | | | | | | | |
| | | | | | | | C99C99. | | | | | | | |
| 748 | 18 | FACVY | NGGVL | TYLESDHFSR | F99F99.A99A99. | N99N99.G99G99. | T88B00.Y99Y99. | 15 | 15 | 30 | 60 | Yes | Yes | Yes |
| | | | | | C99C99.V99V99. | G99G99.V99V99. | L88B00.E88B00. | | | | | | | |
| | | | | | Y99Y99. | L99L99. | S88B00.D88B00. | | | | | | | |
| | | | | | | | H88B00.F88B00. | | | | | | | |
| | | | | | | | S88B00.R83B00. | | | | | | | |
| 146 | 17 | RLCDWIW | NSGST | LHCSLF | R99R99.L99L99. | N99N99.S99S99. | L99L99.H99H99. | 21 | 15 | 18 | 54 | Yes | Yes | Yes |
| | | | | | C99C99.D99D99. | G99G99.S99S99. | C99C99.S99S99. | | | | | | | |
| | | | | | W99W99.I99I99. | T99T99. | L99L99.F99F99. | | | | | | | |
| | | | | | W99W99. | | | | | | | | | |
| 174 | 17 | FSRRYV | MILGS | STVMYSTSL | F99F99.S99S99. | M94B00.I99I99. | S99S99.T52D47. | 21 | 15 | 20 | 56 | Yes | Yes | Yes |
| | | | | | R99R99.R94B00. | L99L99.G99G99. | V99V99.M52S47. | | | | | | | |
| | | | | | Y99Y99.Y99Y99. | S99S99. | Y52E47.S52H47. | | | | | | | |
| | | | | | V99V99. | | T52M41.S52G47. | | | | | | | |
| | | | | | | | L52R47. | | | | | | | |
| 323 | 17 | LDVVGGC | RG | LNCIDCDFA | L99L99.D94B00. | R99R99.G99G99. | L99L99.N99N99. | 21 | 6 | 27 | 54 | Yes | Yes | Yes |
| | | | | | V99V99.V99V99. | | C99C99.I99I99. | | | | | | | |
| | | | | | G99G99.G99G99. | | D99D99.C99C99. | | | | | | | |
| | | | | | C99C99. | | D99D99.F99F99. | | | | | | | |
| | | | | | | | A99A99. | | | | | | | |
| 325 | 17 | DGGFWVA | RTQRF | TTLCPNGWCF | D99D99.G99G99. | R99R99.T99T99. | T99T99.T94B00. | 21 | 15 | 30 | 66 | Yes | Yes | Yes |
| | | | | | G99G99.F99F99. | Q99Q99.R99R99. | L99L99.C94B00. | | | | | | | |
| | | | | | W99W99.V99V99. | F99F99. | P94B00.N94B00. | | | | | | | |
| | | | | | A99A99. | | G99G99.W94B00. | | | | | | | |
| | | | | | | | C99C99.F94B00. | | | | | | | |
| 333 | 17 | LMVVTSF | NQWPA | IDSFGW | L99L99.M99M99. | N99N99.Q99Q99. | I99I99.D99D99. | 21 | 15 | 18 | 54 | Yes | Yes | Yes |
| | | | | | V99V99.V99V99. | W99W99.P99P99. | S99S99.F99F99. | | | | | | | |
| | | | | | T99T99.S94B00. | A99A99. | G99G99.W99W99. | | | | | | | |
| | | | | | F99F99. | | | | | | | | | |
| 338 | 17 | QASEIWL | SSQTF | RYGLGC | Q99Q99.A99A99. | S99S99.S99S99. | R99R99.Y99Y99. | 21 | 15 | 18 | 54 | Yes | Yes | Yes |
| | | | | | S99S99.E99E99. | Q99Q99.T99T99. | G99G99.L99L99. | | | | | | | |
| | | | | | I99I99.W99W99. | E99E99. | G99G99.C99C99. | | | | | | | |
| | | | | | L99L99. | | | | | | | | | |
| 358 | 17 | WRCIGGR | PVGRF | WHFPDNGLVE | W99W99.R99R99. | P99P99.V99V99. | W99W99.H99H99. | 21 | 15 | 30 | 66 | Yes | Yes | Yes |
| | | | | | C99C99.I99I99. | G99G99.R99R99. | F99F99.P99P99. | | | | | | | |
| | | | | | G99G99.G99G99. | F99F99. | D99D99.N99N99. | | | | | | | |
| | | | | | R99R99. | | G99G99.L99L99. | | | | | | | |
| | | | | | | | V99V99.E99E99. | | | | | | | |
| 367 | 17 | YYMHIEF | | LITHDTGVAF | Y99Y99.Y99Y99. | | L99L99.I99I99. | 21 | 0 | 30 | 51 | Yes | Yes | Yes |
| | | | | | M99M99.H99H99. | | T99T99.H99H99. | | | | | | | |
| | | | | | I99I99.E99E99. | | D99D99.T99T99. | | | | | | | |
| | | | | | F99F99. | | G99G99.V99V99. | | | | | | | |
| | | | | | | | A99A99.F99F99. | | | | | | | |
| 462 | 17 | VFFSVDR | PSSCR | LIAVSEDVLY | V99V99.F99F99. | P99P99.S99S99. | L99L99.I99I99. | 21 | 15 | 30 | 66 | Yes | Yes | Yes |
| | | | | | F99F99.S99S99. | S99S99.C99C99. | A99A99.V99V99. | | | | | | | |
| | | | | | V99V99.D99D99. | R99R99. | S99S99.E99E99. | | | | | | | |
| | | | | | R99R99. | | D99D99.V99V99. | | | | | | | |
| | | | | | | | L99L99.Y99Y99. | | | | | | | |
| 512 | 17 | LLGAPYY | KFYRG | TSLDADGTYF | L94B00.L99L99. | K99K99.F99F99. | T99T99.S99S99. | 21 | 15 | 30 | 66 | Yes | Yes | Yes |
| | | | | | G99G99.A99A99. | Y99Y99.R99R99. | L99L99.D99D99. | | | | | | | |
| | | | | | P99P99.Y99Y99. | G99G99. | A99A99.D99D99. | | | | | | | |
| | | | | | Y99Y99. | | G99G99.T99T99. | | | | | | | |
| | | | | | | | Y99Y99.F99F99. | | | | | | | |
| 609 | 17 | LHSCWIR | RRPVL | LRVITHYGVW | L99L99.H99H99. | R99R99.R99R99. | L94B00.R94B00. | 21 | 15 | 30 | 66 | Yes | Yes | Yes |
| | | | | | S99S99.C99C99. | P99P99.V99V99. | V94B00.I94B00. | | | | | | | |
| | | | | | W99W99.I99I99. | L99L99. | T94B00.H94B00. | | | | | | | |
| | | | | | R99R99. | | Y94B00.G94B00. | | | | | | | |
| | | | | | | | V94B00.W94B00. | | | | | | | |
| 69 | 17 | YKLSYLA | GSMLH | SYAMHQCIEY | Y99Y99.K99K99. | G99G99.S99S99. | S94B00.Y99Y99. | 21 | 15 | 30 | 66 | Yes | Yes | Yes |
| | | | | | L99L99.S99S99. | M99M99.L99L99. | A99A99.M99M99. | | | | | | | |
| | | | | | Y99Y99.L99L99. | H99H99. | H99H99.Q99Q99. | | | | | | | |
| | | | | | A99A99. | | C99C99.I99I99. | | | | | | | |
| | | | | | | | E99E99.Y99Y99. | | | | | | | |
| 704 | 17 | DRRYNVW | ISASI | AYFVNGAVI | D99D99.R94B00. | I99I99.S99S99. | A99A99.Y99Y99. | 21 | 15 | 27 | 63 | Yes | Yes | Yes |
| | | | | | R99R99.Y99Y99. | A99A99.S99S99. | F99F99.V99V99. | | | | | | | |
| | | | | | N99N99.V99V99. | I99I99. | N99N99.G99G99. | | | | | | | |
| | | | | | W99W99. | | A99A99.V99V99. | | | | | | | |
| | | | | | | | I99I99. | | | | | | | |
| 717 | 17 | HSYLPLS | EFGEC | VCYNYNGGDDPCR | H99H99.S99S99. | E99E99.F99F99. | V99V99.C99C99. | 21 | 15 | 39 | 75 | Yes | Yes | Yes |
| | | | | | Y99Y99.L99L99. | G99G99.E99E99. | Y99Y99.N99N99. | | | | | | | |
| | | | | | P99P99.L99L99. | C99C99. | Y99Y99.N99N99. | | | | | | | |
| | | | | | S99S99. | | G99G99.G99G99. | | | | | | | |
| | | | | | | | D99D99.D99D99. | | | | | | | |
| | | | | | | | P99P99.C99C99. | | | | | | | |
| | | | | | | | R99R99. | | | | | | | |
| 787 | 17 | TIYRIIY | YGHGP | VQFLGA | T99T99.I99I99. | Y99Y99.G99G99. | V99V99.Q99Q99. | 21 | 15 | 18 | 54 | Yes | Yes | Yes |
| | | | | | Y99Y99.R99R99. | H99H99.G99G99. | F99F99.L99L99. | | | | | | | |
| | | | | | I99I99.I94B00. | P99P99. | G99G99.A99A99. | | | | | | | |
| | | | | | Y94B00. | | | | | | | | | |
| 796 | 17 | VCCLVTV | SDRGV | TTRFSH | V99V99.C99C99. | S99S99.D99D99. | T99T99.T99T99. | 21 | 15 | 18 | 54 | Yes | Yes | Yes |
| | | | | | C99C99.L99L99. | R99R99.G99G99. | R99R99.F99F99. | | | | | | | |
| | | | | | V99V99.T99T99. | V99V99. | S99S99.H99H99. | | | | | | | |
| | | | | | V99V99. | | | | | | | | | |
| 153 | 16 | SISMR | VLNHH | EQLYFDPWGLNVV | S99S99.I99I99. | V99V99.L99L99. | E99E99.Q99Q99. | 15 | 15 | 39 | 69 | Yes | Yes | Yes |
| | | | | | S99S99.M99M99. | N99N99.H99H99. | L99L99.Y99Y99. | | | | | | | |
| | | | | | R99R99. | H99H99. | F99F99.D99D99. | | | | | | | |
| | | | | | | | P99P99.W99W99. | | | | | | | |
| | | | | | | | G99G99.L99L99. | | | | | | | |
| | | | | | | | N99N99.V99V99. | | | | | | | |
| | | | | | | | V99V99. | | | | | | | |
| 255 | 16 | NI | FWDRV | IRFVSSGFD | N99N99.I99I99. | F99F99.W99W99. | I99I99.R99R99. | 6 | 15 | 27 | 48 | Yes | Yes | Yes |
| | | | | | | D99D99.R99R99. | F99F99.V99V99. | | | | | | | |
| | | | | | | V99V99. | S99S99.S99S99. | | | | | | | |
| | | | | | | | G99G99.F99F99. | | | | | | | |
| | | | | | | | D99D99. | | | | | | | |
| 271 | 16 | YVHFSVL | IRDVC | HISFFNGTIVSSY | Y93B00.V93B00. | I99I99.R99R99. | H99H99.I99I99. | 21 | 15 | 39 | 75 | Yes | Yes | Yes |
| | | | | | H93B00.F93B00. | D99D99.V99V99. | S99S99.F99F99. | | | | | | | |
| | | | | | S93B00.V93B00. | C99C99. | F99F99.N99N99. | | | | | | | |
| | | | | | L93B00. | | G99G99.T99T99. | | | | | | | |
| | | | | | | | I99I99.V99V99. | | | | | | | |
| | | | | | | | S99S99.S99S99. | | | | | | | |
| | | | | | | | Y99Y99. | | | | | | | |
| 308 | 16 | DRIVVLG | WSTCE | HIFDQYEGCS | D99D99.R99R99. | W99W99.S99S99. | H99H99.I99I99. | 21 | 15 | 30 | 66 | Yes | Yes | Yes |
| | | | | | I99I99.V99V99. | T93B00.C99C99. | F99F99.D99D99. | | | | | | | |
| | | | | | V99V99.L99L99. | E99E99. | Q99Q99.Y99Y99. | | | | | | | |
| | | | | | G99G99. | | E99E99.G99G99. | | | | | | | |
| | | | | | | | C99C99.S99S99. | | | | | | | |
| 360 | 16 | PSVSYVA | RSWFV | SYIGLGTVV | P99P99.S99S99. | R99R99.S99S99. | S99S99.Y93B00. | 21 | 15 | 27 | 63 | Yes | Yes | Yes |
| | | | | | V99V99.S99S99. | W99W99.F99F99. | I93B00.G93B00. | | | | | | | |
| | | | | | Y99Y99.V99V99. | V99V99. | L93B00.G93B00. | | | | | | | |
| | | | | | A99A99. | | T93B00.V93B00. | | | | | | | |
| | | | | | | | V93B00. | | | | | | | |
| 468 | 16 | GSDLGCW | WFMGA | HSLSSYSGFVYLC | G99G99.S99S99. | W99W99.F99F99. | H99H99.S99S99. | 21 | 15 | 39 | 75 | Yes | Yes | Yes |
| | | | | | D99D99.L99L99. | M99M99.G99G99. | L99L99.S99S99. | | | | | | | |
| | | | | | G99G99.C99C99. | A99A99. | S99S99.Y99Y99. | | | | | | | |
| | | | | | W99W99. | | S99S99.G99G99. | | | | | | | |
| | | | | | | | F99F99.V99V99. | | | | | | | |
| | | | | | | | Y99Y99.L99L99. | | | | | | | |
| | | | | | | | C99C99. | | | | | | | |
| 55 | 16 | VGFHWFA | NPSYD | YVFCHPCMVA | V99V99.G99G99. | N99N99.P99P99. | Y99Y99.V99V99. | 21 | 15 | 30 | 66 | Yes | Yes | Yes |
| | | | | | F99F99.H99H99. | S99S99.Y99Y99. | F99F99.C99C99. | | | | | | | |
| | | | | | W99W99.F99F99. | D99D99. | H99H99.P99P99. | | | | | | | |
| | | | | | A99A99. | | C99C99.M99M99. | | | | | | | |
| | | | | | | | V99V99.A99A99. | | | | | | | |
| 576 | 16 | SRASDTS | YCGIW | GPYIYYHRV | S68B00.R75B00. | Y75B00.C75B00. | G99G99.P99P99. | 14 | 12 | 24 | 50 | Yes | Yes | Yes |
| | | | | | A75B00.S75B00. | G99G99.I99I99. | Y99Y99.I99I99. | | | | | | | |
| | | | | | D75B00.T75B00. | W75B00. | Y99Y99.Y99Y99. | | | | | | | |
| | | | | | S75B00. | | H75B00.R75B00. | | | | | | | |
| | | | | | | | V75B00. | | | | | | | |
| 586 | 16 | CCRSVVW | CGGFA | ACFTANRGVS | C56S37.C99C99. | C99C99.G99G99. | A99A99.C99C99. | 20 | 15 | 30 | 65 | Yes | Yes | Yes |
| | | | | | R93B00.S99S99. | G99G99.F99F99. | F99F99.T99T99. | | | | | | | |
| | | | | | V99V99.V99V99. | A99A99. | A99A99.N99N99. | | | | | | | |
| | | | | | W99W99. | | R99R99.G99G99. | | | | | | | |
| | | | | | | | V99V99.S99S99. | | | | | | | |
| 588 | 16 | GITNNVT | RSVEW | QTSCET | G99G99.I99I99. | R99R99.S99S99. | Q99Q99.T99T99. | 21 | 15 | 18 | 54 | Yes | Yes | Yes |
| | | | | | T81B00.N99N99. | V99V99.E99E99. | S99S99.C99C99. | | | | | | | |
| | | | | | N99N99.V99V99. | W99W99. | E99E99.T99T99. | | | | | | | |
| | | | | | T99T99. | | | | | | | | | |
| 670 | 16 | SVIYICE | RVGPV | AIKSRYVTVP | S99S99.V99V99. | R99R99.V99V99. | A99A99.I99I99. | 21 | 15 | 30 | 66 | Yes | Yes | Yes |
| | | | | | I93B00.Y99Y99. | G99G99.P99P99. | K99K99.S99S99. | | | | | | | |
| | | | | | I99I99.C99C99. | V99V99. | R99R99.Y99Y99. | | | | | | | |
| | | | | | E99E99. | | V99V99.T99T99. | | | | | | | |
| | | | | | | | V99V99.P99P99. | | | | | | | |
| 700 | 16 | SGARRVF | SGALS | VNLDSDMCCR | S99S99.G99G99. | S99S99.G99G99. | V99V99.N99N99. | 21 | 15 | 30 | 66 | Yes | Yes | Yes |
| | | | | | A99A99.R99R99. | A93B00.L99L99. | L99L99.D99D99. | | | | | | | |
| | | | | | R99R99.V99V99. | S99S99. | S99S99.D99D99. | | | | | | | |
| | | | | | F99F99. | | M99M99.C99C99. | | | | | | | |
| | | | | | | | C99C99.R99R99. | | | | | | | |
| 747 | 16 | LYHGLVF | NGDLC | CLLSGEYVA | L99L99.Y99Y99. | N99N99.G99G99. | C99C99.L99L99. | 21 | 15 | 27 | 63 | Yes | Yes | Yes |
| | | | | | H99H99.G99G99. | D99D99.L99L99. | L99L99.S99S99. | | | | | | | |
| | | | | | L99L99.V99V99. | C99C99. | G99G99.E99E99. | | | | | | | |
| | | | | | F99F99. | | Y99Y99.V99V99. | | | | | | | |
| | | | | | | | A99A99. | | | | | | | |
| 77 | 16 | PRTGGVY | AHESH | AYMFHNCGVK | P99P99.R99R99. | A99A99.H99H99. | A99A99.Y99Y99. | 21 | 15 | 30 | 66 | Yes | Yes | Yes |
| | | | | | T99T99.G99G99. | E99E99.S99S99. | M99M99.F99F99. | | | | | | | |
| | | | | | G99G99.V99V99. | H99H99. | H99H99.N99N99. | | | | | | | |
| | | | | | Y99Y99. | | C99C99.G99G99. | | | | | | | |
| | | | | | | | V99V99.K99K99. | | | | | | | |
| 8 | 16 | FHAIARA | NCSCR | YHVTGNTVLI | F93B00.H93B00. | N93B00.C93B00. | Y99Y99.H99H99. | 21 | 15 | 30 | 66 | Yes | Yes | Yes |
| | | | | | A93B00.I93B00. | S99S99.C93B00. | V99V99.T99T99. | | | | | | | |
| | | | | | A93B00.R93B00. | R93B00. | G99G99.N99N99. | | | | | | | |
| | | | | | A93B00. | | T99T99.V99V99. | | | | | | | |
| | | | | | | | L99L99.I99I99. | | | | | | | |
| 85 | 16 | QQLRCVN | GWNFT | VFVNDCSGCNTVI | Q99Q99.Q99Q99. | G99G99.W99W99. | B00 | 21 | 15 | 0 | 36 | Yes | Yes | Yes |
| | | | | | L99L99.R99R99. | N99N99.F99F99. | | | | | | | | |
| | | | | | C99C99.V99V99. | T99T99. | | | | | | | | |
| | | | | | N99N99. | | | | | | | | | |
| 895 | 16 | VGAIVLC | RSKWV | VVLHANACVL | V99V99.G99G99. | R99R99.S99S99. | V99V99.V99V99. | 21 | 15 | 30 | 66 | Yes | Yes | Yes |
| | | | | | A99A99.I99I99. | K99K99.W99W99. | L99L99.H99H99. | | | | | | | |
| | | | | | V99V99.L99L99. | V99V99. | A99A99.N99N99. | | | | | | | |
| | | | | | C99C99. | | A99A99.C93B00. | | | | | | | |
| | | | | | | | V99V99.L99L99. | | | | | | | |
| 94 | 16 | VSSSFPL | FTGFM | DSGLMPWKHG | V56B00.S56B00. | F56B00.T56B00. | D99D99.S99S99. | 14 | 10 | 30 | 54 | Yes | Yes | Yes |
| | | | | | S56B00.S56B00. | G68B00.F56B00. | G99G99.L99L99. | | | | | | | |
| | | | | | F56B00.P68B00. | M56B00. | M99M99.P99P99. | | | | | | | |
| | | | | | L56B00. | | W99W99.K99K99. | | | | | | | |
| | | | | | | | H99H99.G99G99. | | | | | | | |
| 117 | 15 | TVNALCY | VGGDY | ARFIHPVVV | T99T99.V99V99. | V99V99.G99G99. | A99A99.R99R99. | 21 | 15 | 27 | 63 | Yes | Yes | Yes |
| | | | | | N99N99.A93B00. | G99G99.D93B00. | F99F99.I99I99. | | | | | | | |
| | | | | | L99L99.C99C99. | Y99Y99. | H99H99.P99P99. | | | | | | | |
| | | | | | Y99Y99. | | V99V99.V99V99. | | | | | | | |
| | | | | | | | V99V99. | | | | | | | |
| 164 | 15 | VNCFWHS | FGASR | MMVDQFSVCC | V99V99.N99N99. | F99F99.G99G99. | M73B00.M73B00. | 21 | 15 | 20 | 56 | Yes | Yes | Yes |
| | | | | | C99C99.F99F99. | A99A99.S99S99. | V73B00.D73B00. | | | | | | | |
| | | | | | W99W99.H99H99. | R99R99. | Q73B00.F73B00. | | | | | | | |
| | | | | | S99S99. | | S73B00.V73B00. | | | | | | | |
| | | | | | | | C73B00.C73B00. | | | | | | | |
| 20 | 15 | DKPLRTP | ARGLV | NSITHT | D99D99.K99K99. | A99A99.R99R99. | N60B00.S66B00. | 21 | 15 | 12 | 48 | Yes | Yes | Yes |
| | | | | | P99P99.L99L99. | G99G99.L99L99. | I60B00.T60B00. | | | | | | | |
| | | | | | R99R99.T99T99. | V99V99. | H60B00.T60B00. | | | | | | | |
| | | | | | P99P99. | | | | | | | | | |
| 254 | 15 | FGLIRYP | CGIRH | FICAED | F99F99.G99G99. | C99C99.G99G99. | F99F99.I99I99. | 21 | 15 | 18 | 54 | Yes | Yes | Yes |
| | | | | | L99L99.I99I99. | I99I99.R99R99. | C99C99.A99A99. | | | | | | | |
| | | | | | R99R99.Y99Y99. | H99H99. | E99E99.D99D99. | | | | | | | |
| | | | | | P99P99. | | | | | | | | | |
| 344 | 15 | YISALVF | | VNLVAGDCV | Y99Y99.I99I99. | | V99V99.N99N99. | 21 | 0 | 27 | 48 | Yes | Yes | Yes |
| | | | | | S99S99.A99A99. | | L99L99.V99V99. | | | | | | | |
| | | | | | L99L99.V99V99. | | A99A99.G99G99. | | | | | | | |
| | | | | | F99F99. | | D99D99.C99C99. | | | | | | | |
| | | | | | | | V99V99. | | | | | | | |
| 372 | 15 | VFWGI | DMSTT | LAPMIDYF | V99V99.F99F99. | D99D99.M99M99. | L99L99.A99A99. | 21 | 15 | 24 | 60 | Yes | Yes | Yes |
| | | | | | P99P99.N99N99. | S99S99.T99T99. | P99P99.M99M99. | | | | | | | |
| | | | | | W99W99.G99G99. | T99T99. | I99I99.D99D99. | | | | | | | |
| | | | | | I99I99. | | Y99Y99.F99F99. | | | | | | | |
| 402 | 15 | VWLRSVY | LGVCS | ATLDSLGSVPTVS | V99V99.W99W99. | L99L99.G99G99. | A99A99.T99T99. | 21 | 15 | 39 | 75 | Yes | Yes | Yes |
| | | | | | L99L99.R99R99. | V99V99.C99C99. | L99L99.D99D99. | | | | | | | |
| | | | | | S99S99.V99V99. | S99S99. | S99S99.L99L99. | | | | | | | |
| | | | | | Y99Y99. | | G99G99.S99S99. | | | | | | | |
| | | | | | | | V99V99.P99P99. | | | | | | | |
| | | | | | | | T99T99.V99V99. | | | | | | | |
| | | | | | | | S99S99. | | | | | | | |
| 414 | 15 | TWSFMLV | GYRQH | FCFSTDLGVV | T93B00.W93B00. | G99G99.Y99Y99. | F99F99.C99C99. | 21 | 15 | 30 | 66 | Yes | Yes | Yes |
| | | | | | S93B00.F93B00. | R99R99.Q99Q99. | F99F99.S99S99. | | | | | | | |
| | | | | | M93B00.L93B00. | H99H99. | T99T99.D99D99. | | | | | | | |
| | | | | | V93B00. | | L99L99.G99G99. | | | | | | | |
| | | | | | | | V99V99.V99V99. | | | | | | | |
| 442 | 15 | RFLWCVY | PANGE | VCLAHYECLL | R99R99.F93B00. | P99P99.A99A99. | V99V99.C99C99. | 21 | 15 | 30 | 66 | Yes | Yes | Yes |
| | | | | | L93B00.W93B00. | N99N99.G99G99. | L99L99.A99A99. | | | | | | | |
| | | | | | C93B00.V99V99. | E99E99. | H99H99.Y99Y99. | | | | | | | |
| | | | | | Y93B00. | | E99E99.C99C99. | | | | | | | |
| | | | | | | | L99L99.L99L99. | | | | | | | |
| 444 | 15 | LQDVTWI | ASEVW | HIFTGVKSR | L99L99.Q99Q99. | A99A99.S99S99. | H99H99.I99I99. | 21 | 15 | 27 | 63 | Yes | Yes | Yes |
| | | | | | D99D99.V99V99. | E99E99.V99V99. | F99F99.T99T99. | | | | | | | |
| | | | | | T99T99.W99W99. | W99W99. | G99G99.V99V99. | | | | | | | |
| | | | | | I99I99. | | K99K99.S99S99. | | | | | | | |
| | | | | | | | R99R99. | | | | | | | |
| 461 | 15 | GNRHVYA | DSVFR | TLHTDLWGAQ | G99G99.N93B00. | D99D99.S99S99. | T99T99.L99L99. | 21 | 15 | 30 | 66 | Yes | Yes | Yes |
| | | | | | R99R99.H99H99. | V93B00.F99F99. | H99H99.T99T99. | | | | | | | |
| | | | | | V99V99.Y99Y99. | R99R99. | D99D99.L99L99. | | | | | | | |
| | | | | | A99A99. | | W99W99.G99G99. | | | | | | | |
| | | | | | | | A99A99.Q99Q99. | | | | | | | |
| 464 | 15 | LFNPTIG | IRWTA | LATDPTWGSE | L99L99.F99F99. | I99I99.R99R99. | L99L99.A99A99. | 21 | 15 | 30 | 66 | Yes | Yes | Yes |
| | | | | | N93B00.P99P99. | W99W99.T99T99. | T99T99.D99D99. | | | | | | | |
| | | | | | T99T99.I99I99. | A99A99. | P99P99.T99T99. | | | | | | | |
| | | | | | G99G99. | | W99W99.G99G99. | | | | | | | |
| | | | | | | | S99S99.E99E99. | | | | | | | |
| 476 | 15 | SFRWGEE | HDCLP | H | S99S99.F99F99. | H99H99.D99D99. | H99H99. | 21 | 15 | 3 | 39 | Yes | Yes | Yes |
| | | | | | R99R99.W99W99. | C99C99.L99L99. | | | | | | | | |
| | | | | | G99G99.E99E99. | P99P99. | | | | | | | | |
| | | | | | E99E99. | | | | | | | | | |
| 546 | 15 | RLTFLVW | GDGFG | QCFYLGSVT | R93B00.L99L99. | G99G99.D99D99. | Q99Q99.C99C99. | 21 | 15 | 27 | 63 | Yes | Yes | Yes |
| | | | | | T99T99.F99F99. | G99G99.F99F99. | F99F99.Y99Y99. | | | | | | | |
| | | | | | L99L99.V99V99. | G99G99. | L99L99.G99G99. | | | | | | | |
| | | | | | W99W99. | | S99S99.V99V99. | | | | | | | |
| | | | | | | | T99T99. | | | | | | | |
| 653 | 15 | GIHSPLQ | EISCW | LCWSNSGRR | G99G99.I99I99. | E99E99.I99I99. | L99L99.C99C99. | 21 | 15 | 27 | 63 | Yes | Yes | Yes |
| | | | | | H99H99.S99S99. | S99S99.C99C99. | W99W99.S99S99. | | | | | | | |
| | | | | | P99P99.L99L99. | W99W99. | N99N99.S99S99. | | | | | | | |
| | | | | | Q99Q99. | | G99G99.R99R99. | | | | | | | |
| | | | | | | | R99R99. | | | | | | | |
| 682 | 15 | MNIVWSW | LTSLQ | IDIWGC | M99M99.N99N99. | L99L99.T99T99. | I99I99.D99D99. | 21 | 15 | 18 | 54 | Yes | Yes | Yes |
| | | | | | I99I99.V99V99. | S99S99.L99L99. | I99I99.W99W99. | | | | | | | |
| | | | | | W99W99.S99S99. | Q99Q99. | G99G99.C99C99. | | | | | | | |
| | | | | | W99W99. | | | | | | | | | |
| 699 | 15 | CDDDFRV | YGCPS | VTPHM | C93B00.D99D99. | Y99Y99.G99G99. | V93B00.T93B00. | 21 | 15 | 15 | 51 | Yes | Yes | Yes |
| | | | | | D99D99.D99D99. | C99C99.P99P99. | P93B00.H93B00. | | | | | | | |
| | | | | | F99F99.R99R99. | S99S99. | M93B00. | | | | | | | |
| | | | | | V99V99. | | | | | | | | | |
| 734 | 15 | AWR | LSAIC | YDFLTW | A99A99.W99W99. | L99L99.S99S99. | Y99Y99.D99D99. | 9 | 15 | 18 | 42 | Yes | Yes | Yes |
| | | | | | R99R99. | A99A99.I99I99. | F99F99.L99L99. | | | | | | | |
| | | | | | | C99C99. | T99T99.W99W99. | | | | | | | |
| 746 | 15 | LMGRLIF | TRGME | VVFLNHFSNV | L99L99.M99M99. | T99T99.R99R99. | V93B00.V93B00. | 21 | 15 | 30 | 66 | Yes | Yes | Yes |
| | | | | | G99G99.R99R99. | G99G99.M99M99. | F93B00.L93B00. | | | | | | | |
| | | | | | L99L99.I99I99. | E99E99. | N93B00.H93B00. | | | | | | | |
| | | | | | F99F99. | | F93B00.S99S99. | | | | | | | |
| | | | | | | | N93B00.V93B00. | | | | | | | |
| 798 | 15 | SRIRILA | RNPCC | SILAGSTGA | S99S99.R99R99. | R99R99.N99N99. | S80B00.I99I99. | 21 | 15 | 19 | 55 | Yes | Yes | Yes |
| | | | | | I99I99.R99R99. | P99P99.C99C99. | L80B00.A80B00. | | | | | | | |
| | | | | | I99I99.L99L99. | C99C99. | G80B00.S80B00. | | | | | | | |
| | | | | | A99A99. | | T80B00.G80B00. | | | | | | | |
| | | | | | | | A80B00. | | | | | | | |
| 810 | 15 | DYASLVY | NGSGD | AYVSLGSVY | D93B00.Y99Y99. | N99N99.G99G99. | A99A99.Y99Y99. | 21 | 15 | 27 | 63 | Yes | Yes | Yes |
| | | | | | A99A99.S99S99. | S99S99.G99G99. | V99V99.S99S99. | | | | | | | |
| | | | | | L99L99.V99V99. | D99D99. | L99L99.G99G99. | | | | | | | |
| | | | | | Y99Y99. | | S99S99.V99V99. | | | | | | | |
| | | | | | | | Y99Y99. | | | | | | | |
| 847 | 15 | SMCDPRS | TNTYW | QDHWAD | S99S99.M99M99. | T99T99.N99N99. | Q99Q99.D99D99. | 21 | 15 | 18 | 54 | Yes | Yes | Yes |
| | | | | | C99C99.D99D99. | T99T99.Y99Y99. | H99H99.W99W99. | | | | | | | |
| | | | | | P99P99.R99R99. | W99W99. | A99A99.D99D99. | | | | | | | |
| | | | | | S99S99. | | | | | | | | | |
| 92 | 15 | YIDSHEV | KFSFH | VRL | Y99Y99.I99I99. | K99K99.F99F99. | V99V99.R99R99. | 21 | 15 | 0 | 45 | Yes | Yes | Yes |
| | | | | | D99D99.S99S99. | S99S99.F99F99. | L99L99. | | | | | | | |
| | | | | | H99H99.E99E99. | H99H99. | | | | | | | | |
| | | | | | V99V99. | | | | | | | | | |
| 130 | 14 | RPYRGSV | YGSLD | DKFFRAHVV | R99R99.P99P99. | Y99Y99.G99G99. | D99D99.K99K99. | 21 | 15 | 27 | 63 | Yes | Yes | Yes |
| | | | | | Y99Y99.R99R99. | S99S99.L92B00. | F99F99.F99F99. | | | | | | | |
| | | | | | G99G99.S99S99. | D99D99. | R99R99.A99A99. | | | | | | | |
| | | | | | V99V99. | | H99H99.V99V99. | | | | | | | |
| | | | | | | | V99V99. | | | | | | | |
| 138 | 14 | LMHRVVF | NDSSI | VRYGGH | L99L99.M99M99. | N99N99.D99D99. | V99V99.R99R99. | 21 | 15 | 18 | 54 | Yes | Yes | Yes |
| | | | | | H99H99.R99R99. | S99S99.S99S99. | Y99Y99.G99G99. | | | | | | | |
| | | | | | V99V99.V99V99. | I99I99. | G99G99.H99H99. | | | | | | | |
| | | | | | F99F99. | | | | | | | | | |
| 150 | 14 | VCHVVW | GDANR | VLLINMGRLE | V99V99.C99C99. | G99G99.D99D99. | V99V99.L99L99. | 18 | 15 | 30 | 63 | Yes | Yes | Yes |
| | | | | | H99H99.V99V99. | A99A99.N99N99. | L99L99.I99I99. | | | | | | | |
| | | | | | V99V99.W99W99. | R99R99. | N99N99.M99M99. | | | | | | | |
| | | | | | | | G99G99.R99R99. | | | | | | | |
| | | | | | | | L99L99.E99E99. | | | | | | | |
| 218 | 14 | VDARALS | CLFIR | VHPDTC | V99V99.D99D99. | C99C99.L99L99. | V99V99.H99H99. | 21 | 15 | 18 | 54 | Yes | Yes | Yes |
| | | | | | A99A99.R99R99. | F99F99.I99I99. | P99P99.D99D99. | | | | | | | |
| | | | | | A99A99.L99L99. | R99R99. | T99T99.C99C99. | | | | | | | |
| | | | | | S99S99. | | | | | | | | | |
| 285 | 14 | GHAEMYQ | AVSGN | MNWCWRGRC | G99G99.H99H99. | A99A99.V99V99. | M99M99.N99N99. | 21 | 15 | 27 | 63 | Yes | Yes | Yes |
| | | | | | A99A99.E99E99. | S99S99.G99G99. | W99W99.C99C99. | | | | | | | |
| | | | | | M99M99.Y99Y99. | N99N99. | W99W99.R99R99. | | | | | | | |
| | | | | | Q99Q99. | | G99G99.R99R99. | | | | | | | |
| | | | | | | | C99C99. | | | | | | | |
| 369 | 14 | LDSGVVF | SWGTR | SIFIDHGCVD | L99L99.D99D99. | S99S99.W99W99. | S99S99.I99I99. | 21 | 15 | 30 | 66 | Yes | Yes | Yes |
| | | | | | S99S99.G99G99. | G99G99.T99T99. | F99F99.I99I99. | | | | | | | |
| | | | | | V99V99.V99V99. | R99R99. | D99D99.H99H99. | | | | | | | |
| | | | | | F99F99. | | G99G99.C99C99. | | | | | | | |
| | | | | | | | V99V99.D99D99. | | | | | | | |
| 379 | 14 | IRGFIVC | SCPSG | NIHNAGYTD | I99I99.R99R99. | S99S99.C99C99. | N99N99.I99I99. | 21 | 15 | 27 | 63 | Yes | Yes | Yes |
| | | | | | G99G99.F99F99. | P99P99.S99S99. | H99H99.N99N99. | | | | | | | |
| | | | | | I99I99.V99V99. | G99G99. | A99A99.G99G99. | | | | | | | |
| | | | | | C99C99. | | Y99Y99.T99T99. | | | | | | | |
| | | | | | | | D99D99. | | | | | | | |
| 396 | 14 | YASEIRS | AVRPY | FVSCIF | Y92B00.A92B00. | A99A99.V99V99. | F99F99.V99V99. | 21 | 15 | 18 | 54 | Yes | Yes | Yes |
| | | | | | S85B00.E92B00. | R99R99.P99P99. | S99S99.C99C99. | | | | | | | |
| | | | | | I92B00.R92B00. | Y99Y99. | I99I99.F99F99. | | | | | | | |
| | | | | | S92B00. | | | | | | | | | |
| 423 | 14 | RVVDMLH | GFNSL | PIFNSLGLS | R99R99.V99V99. | G99G99.F99F99. | P99P99.I92B00. | 21 | 15 | 27 | 63 | Yes | Yes | Yes |
| | | | | | V99V99.D99D99. | N99N99.S99S99. | F92B00.N92B00. | | | | | | | |
| | | | | | M99M99.L99L99. | L99L99. | S92B00.L92B00. | | | | | | | |
| | | | | | H92B00. | | G92B00.L92B00. | | | | | | | |
| | | | | | | | S92B00. | | | | | | | |
| 440 | 14 | CLDMCYV | RLLHE | WTSCSR | C99C99.L99L99. | R99R99.L99L99. | W99W99.T99T99. | 21 | 15 | 18 | 54 | Yes | Yes | Yes |
| | | | | | D99D99.M99M99. | L99L99.H99H99. | S99S99.C99C99. | | | | | | | |
| | | | | | C99C99.Y99Y99. | E99E99. | S99S99.R99R99. | | | | | | | |
| | | | | | V99V99. | | | | | | | | | |
| 471 | 14 | ICEPMWA | FGSVR | LDSEW | I92B00.C99C99. | F99F99.G99G99. | L99L99.D99D99. | 21 | 15 | 15 | 51 | Yes | Yes | Yes |
| | | | | | E99E99.P99P99. | S92B00.V99V99. | S99S99.E99E99. | | | | | | | |
| | | | | | M99M99.W99W99. | R99R99. | W99W99. | | | | | | | |
| | | | | | A99A99. | | | | | | | | | |
| 547 | 14 | MYIGDVL | GVSGL | CVFVGTCQRR | M92B00.Y99Y99. | G99G99.V99V99. | C99C99.V99V99. | 21 | 15 | 30 | 66 | Yes | Yes | Yes |
| | | | | | I99I99.G99G99. | S99S99.G99G99. | F99F99.V99V99. | | | | | | | |
| | | | | | D99D99.V99V99. | L99L99. | G99G99.T99T99. | | | | | | | |
| | | | | | L99L99. | | C99C99.Q99Q99. | | | | | | | |
| | | | | | | | R99R99.R99R99. | | | | | | | |
| 549 | 14 | VFWTERW | PCSSR | GHVITSVLIF | V99V99.F99F99. | P99P99.C99C99. | G99G99.H99H99. | 21 | 15 | 30 | 66 | Yes | Yes | Yes |
| | | | | | W99W99.T99T99. | S99S99.S99S99. | V99V99.I99I99. | | | | | | | |
| | | | | | E99E99.R99R99. | R99R99. | T99T99.S99S99. | | | | | | | |
| | | | | | W99W99. | | V99V99.L99L99. | | | | | | | |
| | | | | | | | I99I99.F99F99. | | | | | | | |
| 557 | 14 | FSSPWLC | FGSLR | LLLTNTDFGRAAR | F99F99.S99S99. | F99F99.G99G99. | B00 | 21 | 15 | 0 | 36 | Yes | Yes | Yes |
| | | | | | S99S99.P99P99. | S99S99.L99L99. | | | | | | | | |
| | | | | | W99W99.L99L99. | R99R99. | | | | | | | | |
| | | | | | C99C99. | | | | | | | | | |
| 559 | 14 | VCVHCFA | LGGLN | IAMNTAWDVC | V99V99.C99C99. | L99L99.G99G99. | I99I99.A99A99. | 21 | 15 | 30 | 66 | Yes | Yes | Yes |
| | | | | | V99V99.H99H99. | G99G99.L92B00. | M99M99.N99N99. | | | | | | | |
| | | | | | C99C99.F99F99. | N99N99. | T99T99.A99A99. | | | | | | | |
| | | | | | A99A99. | | W99W99.D99D99. | | | | | | | |
| | | | | | | | V99V99.C99C99. | | | | | | | |
| 563 | 14 | SNVPMNS | CNFFF | DCASFVIVGY | S92B00.N92B00. | C99C99.N99N99. | D99D99.C99C99. | 21 | 15 | 30 | 66 | Yes | Yes | Yes |
| | | | | | V99V99.P92B00. | F99F99.F99F99. | A99A99.S99S99. | | | | | | | |
| | | | | | M99M99.N99N99. | F99F99. | F99F99.V99V99. | | | | | | | |
| | | | | | S99S99. | | I99I99.V99V99. | | | | | | | |
| | | | | | | | G99G99.Y99Y99. | | | | | | | |
| 568 | 14 | KLGHVVF | | VACRLYGHSLNLE | K99K99.L99L99. | | V99V99.A99A99. | 21 | 0 | 39 | 60 | Yes | Yes | Yes |
| | | | | | G99G99.H99H99. | | C99C99.R99R99. | | | | | | | |
| | | | | | V99V99.V99V99. | | L99L99.Y99Y99. | | | | | | | |
| | | | | | F99F99. | | G99G99.H99H99. | | | | | | | |
| | | | | | | | S99S99.L99L99. | | | | | | | |
| | | | | | | | N99N99.L99L99. | | | | | | | |
| | | | | | | | E99E99. | | | | | | | |
| 617 | 14 | AFVGCWT | NYPAI | HIMCCHCMPARCF | A92B00.F99F99. | N99N99.Y99Y99. | H99H99.I99I99. | 21 | 15 | 39 | 75 | Yes | Yes | Yes |
| | | | | | V99V99.G99G99. | P99P99.A92B00. | M99M99.C92B00. | | | | | | | |
| | | | | | C99C99.W99W99. | I99I99. | C99C99.H99H99. | | | | | | | |
| | | | | | T99T99. | | C99C99.M99M99. | | | | | | | |
| | | | | | | | P99P99.A92B00. | | | | | | | |
| | | | | | | | R99R99.C99C99. | | | | | | | |
| | | | | | | | F99F99. | | | | | | | |
| 624 | 14 | FDLSWVQ | ALSAH | ILASGAGVS | F99F99.D99D99. | A99A99.L99L99. | I99I99.L99L99. | 21 | 15 | 27 | 63 | Yes | Yes | Yes |
| | | | | | L99L99.S99S99. | S99S99.A99A99. | A99A99.S99S99. | | | | | | | |
| | | | | | W99W99.V99V99. | H99H99. | G99G99.A99A99. | | | | | | | |
| | | | | | Q99Q99. | | G99G99.V99V99. | | | | | | | |
| | | | | | | | S99S99. | | | | | | | |
| 659 | 14 | CSVLRGT | VCAFWW | QHTCEL | C99C99.S99S99. | V99V99.C99C99. | Q99Q99.H99H99. | 21 | 15 | 18 | 54 | Yes | Yes | Yes |
| | | | | | V99V99.L99L99. | A92B00.F99F99. | T99T99.C99C99. | | | | | | | |
| | | | | | R99R99.G99G99. | W99W99. | E99E99.L99L99. | | | | | | | |
| | | | | | T99T99. | | | | | | | | | |
| 675 | 14 | RGFDWFQ | RPVRV | LSILPTSRVG | R99R99.G99G99. | R99R99.P99P99. | L99L99.S99S99. | 21 | 15 | 30 | 66 | Yes | Yes | Yes |
| | | | | | F99F99.D99D99. | V99V99.R99R99. | I99I99.L99L99. | | | | | | | |
| | | | | | W99W99.F99F99. | V99V99. | P99P99.T99T99. | | | | | | | |
| | | | | | Q99Q99. | | S99S99.R99R99. | | | | | | | |
| | | | | | | | V99V99.G99G99. | | | | | | | |
| 703 | 14 | LSCGIGS | KMFAV | SLCNRLEMYI | L85B00.S92B00. | K99K99.M99M99. | S99S99.L99L99. | 21 | 15 | 30 | 66 | Yes | Yes | Yes |
| | | | | | C99C99.G99G99. | F99F99.A99A99. | C99C99.N99N99. | | | | | | | |
| | | | | | I99I99.G99G99. | V99V99. | R99R99.L99L99. | | | | | | | |
| | | | | | S99S99. | | E99E99.M99M99. | | | | | | | |
| | | | | | | | Y99Y99.I99I99. | | | | | | | |
| 737 | 14 | GLRRMGG | WWDLF | VTWSVLGVL | G99G99.L99L99. | W99W99.W99W99. | V99V99.T99T99. | 21 | 15 | 27 | 63 | Yes | Yes | Yes |
| | | | | | R99R99.R99R99. | D99D99.L99L99. | W99W99.S99S99. | | | | | | | |
| | | | | | M99M99.G99G99. | F99F99. | V99V99.L99L99. | | | | | | | |
| | | | | | G99G99. | | G99G99.V99V99. | | | | | | | |
| | | | | | | | L99L99. | | | | | | | |
| 812 | 14 | NLR | RCMCF | LPLFAD | N99N99.L99L99. | R99R99.C99C99. | L99L99.P92B00. | 9 | 15 | 18 | 42 | Yes | Yes | Yes |
| | | | | | R99R99. | M99M99.C99C99. | L92B00.F92B00. | | | | | | | |
| | | | | | | F99F99. | A92B00.D92B00. | | | | | | | |
| 97 | 14 | KPGPMLY | RDPRG | IYWSSGRVC | K99K99.P99P99. | R99R99.D99D99. | I99I99.Y99Y99. | 21 | 15 | 27 | 63 | Yes | Yes | Yes |
| | | | | | G99G99.P99P99. | P99P99.R99R99. | W99W99.S99S99. | | | | | | | |
| | | | | | M99M99.L99L99. | G99G99. | S99S99.G99G99. | | | | | | | |
| | | | | | Y99Y99. | | R99R99.V99V99. | | | | | | | |
| | | | | | | | C99C99. | | | | | | | |
| 119 | 13 | AFGGVCW | WERGN | LLWLCPGSFGEYF | A99A99.F99F99. | W99W99.E99E99. | L99L99.L99L99. | 21 | 15 | 39 | 75 | Yes | Yes | Yes |
| | | | | | G99G99.G99G99. | R99R99.G99G99. | W99W99.L99L99. | | | | | | | |
| | | | | | V99V99.C99C99. | N99N99. | C99C99.P92B00. | | | | | | | |
| | | | | | W99W99. | | G99G99.S99S99. | | | | | | | |
| | | | | | | | F99F99.G99G99. | | | | | | | |
| | | | | | | | E99E99.Y99Y99. | | | | | | | |
| | | | | | | | F99F99. | | | | | | | |
| 194 | 13 | DCLIGGV | SGNGH | DSYARYIFPF | D99D99.C99C99. | S99S99.G99G99. | D99D99.S99S99. | 21 | 15 | 30 | 66 | Yes | Yes | Yes |
| | | | | | L99L99.I99I99. | N99N99.G99G99. | Y99Y99.A99A99. | | | | | | | |
| | | | | | G99G99.G99G99. | H99H99. | R99R99.Y99Y99. | | | | | | | |
| | | | | | V99V99. | | I99I99.F99F99. | | | | | | | |
| | | | | | | | P99P99.F99F99. | | | | | | | |
| 219 | 13 | VSHYWCR | RAASP | SYITHVDNC | V99V99.S99S99. | R99R99.A99A99. | S99S99.Y99Y99. | 21 | 15 | 30 | 66 | Yes | Yes | Yes |
| | | | | | H99H99.Y99Y99. | A99A99.S99S99. | I99I99.T99T99. | | | | | | | |
| | | | | | W99W99.C99C99. | P99P99. | H99H99.T99T99. | | | | | | | |
| | | | | | R99R99. | | V99V99.D99D99. | | | | | | | |
| | | | | | | | N99N99.C99C99. | | | | | | | |
| 251 | 13 | RLHEIFY | CYCAT | AASHHGLNII | R99R99.L99L99. | C99C99.Y99Y99. | A99A99.A99A99. | 21 | 15 | 30 | 66 | Yes | Yes | Yes |
| | | | | | H99H99.E99E99. | C99C99.A99A99. | S99S99.H99H99. | | | | | | | |
| | | | | | I99I99.F99F99. | T99T99. | H99H99.G99G99. | | | | | | | |
| | | | | | Y99Y99. | | L99L99.N99N99. | | | | | | | |
| | | | | | | | I99I99.I99I99. | | | | | | | |
| 305 | 13 | PMLCGFP | FDGLF | FTISYPGYFR | P99P99.M99M99. | F99F99.D99D99. | F99F99.T99T99. | 21 | 15 | 30 | 66 | Yes | Yes | Yes |
| | | | | | L99L99.C99C99. | G99G99.L99L99. | I99I99.S99S99. | | | | | | | |
| | | | | | G99G99.F99F99. | F99F99. | Y99Y99.P99P99. | | | | | | | |
| | | | | | P99P99. | | G99G99.Y99Y99. | | | | | | | |
| | | | | | | | F99F99.R99R99. | | | | | | | |
| 310 | 13 | FIRNCML | TGNRT | IVFNYNNCVD | F99F99.I99I99. | T99T99.G99G99. | I99I99.V99V99. | 21 | 15 | 30 | 66 | Yes | Yes | Yes |
| | | | | | R99R99.N99N99. | N99N99.R99R99. | F99F99.N99N99. | | | | | | | |
| | | | | | C99C99.M99M99. | T99T99. | Y99Y99.N99N99. | | | | | | | |
| | | | | | L99L99. | | N99N99.C99C99. | | | | | | | |
| | | | | | | | V99V99.D99D99. | | | | | | | |
| 342 | 13 | PVLRVTF | SGEYL | VMLDKQMHTC | P99P99.V99V99. | S99S99.G99G99. | V99V99.M99M99. | 21 | 15 | 30 | 66 | Yes | Yes | Yes |
| | | | | | L99L99.R99R99. | E99E99.Y99Y99. | L99L99.D99D99. | | | | | | | |
| | | | | | V99V99.T99T99. | L99L99. | K99K99.Q99Q99. | | | | | | | |
| | | | | | F99F99. | | M99M99.H99H99. | | | | | | | |
| | | | | | | | T92B00.C99C99. | | | | | | | |
| 346 | 13 | VARFRGT | IISPA | LAYPVESCFD | V99V99.A99A99. | I99I99.I99I99. | L99L99.A99A99. | 21 | 15 | 30 | 66 | Yes | Yes | Yes |
| | | | | | R99R99.F99F99. | S99S99.P99P99. | Y99Y99.P99P99. | | | | | | | |
| | | | | | R99R99.G99G99. | A99A99. | V99V99.E99E99. | | | | | | | |
| | | | | | T92B00. | | S99S99.C99C99. | | | | | | | |
| | | | | | | | F99F99.D99D99. | | | | | | | |
| 361 | 13 | DVDHVFV | SYVER | NIFRDDCNR | D99D99.V99V99. | S99S99.Y99Y99. | N99N99.I99I99. | 21 | 15 | 27 | 63 | Yes | Yes | Yes |
| | | | | | D99D99.H99H99. | V99V99.E99E99. | F99F99.R99R99. | | | | | | | |
| | | | | | V99V99.F99F99. | R99R99. | D99D99.D99D99. | | | | | | | |
| | | | | | V99V99. | | C99C99.N99N99. | | | | | | | |
| | | | | | | | R99R99. | | | | | | | |
| 390 | 13 | ILGILAY | FAPDA | LSRFSH | I99I99.L99L99. | F99F99.A99A99. | L99L99.S99S99. | 21 | 15 | 18 | 54 | Yes | Yes | Yes |
| | | | | | G99G99.I99I99. | P99P99.D99D99. | R99R99.F99F99. | | | | | | | |
| | | | | | L99L99.A99A99. | A99A99. | S99S99.H99H99. | | | | | | | |
| | | | | | Y99Y99. | | | | | | | | | |
| 405 | 13 | PGRRTIS | CLDWC | TTLFTDCRCQ | P99P99.G92B00. | C99C99.L99L99. | T99T99.T99T99. | 21 | 15 | 30 | 66 | Yes | Yes | Yes |
| | | | | | R99R99.R99R99. | D99D99.W99W99. | L99L99.F99F99. | | | | | | | |
| | | | | | T99T99.I99I99. | C99C99. | T99T99.D99D99. | | | | | | | |
| | | | | | S92B00. | | C99C99.R99R99. | | | | | | | |
| | | | | | | | C99C99.Q99Q99. | | | | | | | |
| 430 | 13 | INACYEH | PIPV | YKLKQS | I99I99.N92B00. | P99P99.I99I99. | Y76B00.K53B00. | 21 | 15 | 12 | 48 | Yes | Yes | Yes |
| | | | | | A99A99.C99C99. | A99A99.P99P99. | L76B00.K53B00. | | | | | | | |
| | | | | | Y99Y99.E99E99. | V99V99. | Q53B00.S53B00. | | | | | | | |
| | | | | | H84B00. | | | | | | | | | |
| 482 | 13 | GDSLFGV | KATAR | ASSFEVCMAMGLC | G99G99.D99D99. | K99K99.A99A99. | A99A99.S99S99. | 21 | 15 | 39 | 75 | Yes | Yes | Yes |
| | | | | | S99S99.L99L99. | T99T99.A99A99. | S99S99.F99F99. | | | | | | | |
| | | | | | F99F99.G99G99. | R99R99. | E99E99.V99V99. | | | | | | | |
| | | | | | V99V99. | | C99C99.M99M99. | | | | | | | |
| | | | | | | | A99A99.M99M99. | | | | | | | |
| | | | | | | | G99G99.L99L99. | | | | | | | |
| | | | | | | | C99C99. | | | | | | | |
| 503 | 13 | DEFRRSG | CLMEY | NSGYGY | D99D99.E99E99. | C99C99.L99L99. | N99N99.S99S99. | 21 | 15 | 18 | 54 | Yes | Yes | Yes |
| | | | | | F99F99.R99R99. | M99M99.E99E99. | G99G99.Y99Y99. | | | | | | | |
| | | | | | R99R99.S99S99. | Y99Y99. | G99G99.Y99Y99. | | | | | | | |
| | | | | | G99G99. | | | | | | | | | |
| 504 | 13 | TRDCGAS | PVFAC | YFSLEC | T99T99.R99R99. | P99P99.V99V99. | Y99Y99.F99F99. | 21 | 15 | 18 | 54 | Yes | Yes | Yes |
| | | | | | D99D99.C99C99. | F99F99.A99A99. | S99S99.L99L99. | | | | | | | |
| | | | | | G99G99.A99A99. | C99C99. | E99E99.C99C99. | | | | | | | |
| | | | | | S99S99. | | | | | | | | | |
| 543 | 13 | NLDSIVF | IACGP | MKYHKSGDMV | N99N99.L99L99. | I99I99.A99A99. | M99M99.K99K99. | 21 | 15 | 30 | 66 | Yes | Yes | Yes |
| | | | | | D99D99.S99S99. | C99C99.G99G99. | Y99Y99.H99H99. | | | | | | | |
| | | | | | I99I99.V99V99. | P99P99. | K99K99.S99S99. | | | | | | | |
| | | | | | F99F99. | | G99G99.D99D99. | | | | | | | |
| | | | | | | | M99M99.V99V99. | | | | | | | |
| 554 | 13 | KRRVLVE | SWCGS | FYPIPNLVCA | K99K99.R99R99. | S99S99.W99W99. | F99F99.Y99Y99. | 21 | 15 | 30 | 66 | Yes | Yes | Yes |
| | | | | | R99R99.V92B00. | C99C99.G99G99. | P99P99.I99I99. | | | | | | | |
| | | | | | L99L99.V99V99. | S99S99. | P99P99.N99N99. | | | | | | | |
| | | | | | E99E99. | | L99L99.V99V99. | | | | | | | |
| | | | | | | | C99C99.A99A99. | | | | | | | |
| 57 | 13 | ARRCMLW | PNIDG | VLYVNGLAA | A99A99.R99R99. | P99P99.N99N99. | V99V99.L99L99. | 21 | 15 | 27 | 63 | Yes | Yes | Yes |
| | | | | | R99R99.C99C99. | I99I99.D99D99. | Y99Y99.V99V99. | | | | | | | |
| | | | | | M99M99.L99L99. | G99G99. | N99N99.G92B00. | | | | | | | |
| | | | | | W99W99. | | L99L99.A99A99. | | | | | | | |
| | | | | | | | A99A99. | | | | | | | |
| 636 | 13 | HVARVVF | ACQSL | IANGLTTRML | H99H99.V99V99. | A99A99.C99C99. | I99I99.A99A99. | 21 | 15 | 30 | 66 | Yes | Yes | Yes |
| | | | | | A99A99.R99R99. | Q99Q99.S99S99. | N99N99.G99G99. | | | | | | | |
| | | | | | V99V99.V99V99. | L99L99. | L99L99.T99T99. | | | | | | | |
| | | | | | F99F99. | | T99T99.R99R99. | | | | | | | |
| | | | | | | | M99M99.L99L99. | | | | | | | |
| 73 | 13 | SPFGGQ | SDYHV | LNSTCLGVRW | S99S99.I99I99. | S99S99.D99D99. | L99L99.N99N99. | 21 | 15 | 30 | 66 | Yes | Yes | Yes |
| | | | | | P99P99.F99F99. | Y99Y99.H99H99. | S99S99.T99T99. | | | | | | | |
| | | | | | G99G99.G99G99. | V99V99. | C99C99.L99L99. | | | | | | | |
| | | | | | Q99Q99. | | G99G99.V99V99. | | | | | | | |
| | | | | | | | R99R99.W99W99. | | | | | | | |
| 743 | 13 | CIVGYPC | YYILA | FASQWV | C99C99.I99I99. | Y99Y99.Y99Y99. | F99F99.A99A99. | 21 | 15 | 18 | 54 | Yes | Yes | Yes |
| | | | | | V99V99.G99G99. | I99I99.L99L99. | S99S99.Q99Q99. | | | | | | | |
| | | | | | Y99Y99.P99P99. | A99A99. | W99W99.V99V99. | | | | | | | |
| | | | | | C99C99. | | | | | | | | | |
| 759 | 13 | R | WVNGP | APWRVD | R99R99. | W99W99.V99V99. | A99A99.P99P99. | 3 | 15 | 18 | 36 | Yes | Yes | Yes |
| | | | | | | N99N99.G99G99. | W99W99.R92B00. | | | | | | | |
| | | | | | | P99P99. | V99V99.D99D99. | | | | | | | |
| 766 | 13 | TLTGVVY | VGSVE | ITFCLT | T99T99.L99L99. | V99V99.G99G99. | I99I99.T99T99. | 21 | 15 | 18 | 54 | Yes | Yes | Yes |
| | | | | | T99T99.G99G99. | S99S99.V99V99. | F99F99.C99C99. | | | | | | | |
| | | | | | V99V99.V99V99. | E99E99. | L99L99.T99T99. | | | | | | | |
| | | | | | Y99Y99. | | | | | | | | | |
| 774 | 13 | NRPHVCQ | ALGDF | LTTSPNRVFW | N99N99.R99R99. | A99A99.L99L99. | L99L99.T99T99. | 21 | 15 | 30 | 66 | Yes | Yes | Yes |
| | | | | | P99P99.H99H99. | G99G99.D99D99. | T99T99.S99S99. | | | | | | | |
| | | | | | V99V99.C99C99. | F99F99. | P99P99.N99N99. | | | | | | | |
| | | | | | Q99Q99. | | R99R99.V92B00. | | | | | | | |
| | | | | | | | F99F99.W99W99. | | | | | | | |
| 797 | 13 | VVDLLIS | KDLSA | PTRFGA | V99V99.V99V99. | K99K99.D99D99. | P99P99.T99T99. | 21 | 15 | 18 | 54 | Yes | Yes | Yes |
| | | | | | D99D99.L99L99. | L99L99.S99S99. | R99R99.F99F99. | | | | | | | |
| | | | | | L99L99.I99I99. | A99A99. | G99G99.A99A99. | | | | | | | |
| | | | | | S99S99. | | | | | | | | | |
| 800 | 13 | TQVRPAC | PLRGD | RIYIELMIYWGLV | T99T99.Q92B00. | P99P99.L99L99. | R99R99.I99I99. | 21 | 15 | 39 | 75 | Yes | Yes | Yes |
| | | | | | V99V99.R99R99. | R99R99.G99G99. | Y99Y99.I99I99. | | | | | | | |
| | | | | | P99P99.A99A99. | D99D99. | E99E99.L99L99. | | | | | | | |
| | | | | | C99C99. | | M99M99.I99I99. | | | | | | | |
| | | | | | | | Y99Y99.W99W99. | | | | | | | |
| | | | | | | | G99G99.L99L99. | | | | | | | |
| | | | | | | | V99V99. | | | | | | | |
| 809 | 13 | YHLEMSG | GSFLR | HPEGSW | Y99Y99.H92B00. | G99G99.S99S99. | H99H99.P99P99. | 21 | 15 | 18 | 54 | Yes | Yes | Yes |
| | | | | | L99L99.E99E99. | F99F99.L99L99. | E99E99.G99G99. | | | | | | | |
| | | | | | M99M99.S99S99. | R99R99. | S99S99.W99W99. | | | | | | | |
| | | | | | G99G99. | | | | | | | | | |
| 89 | 13 | FIDFGQS | SEGSR | VAFYSK | F99F99.I99I99. | S99S99.E99E99. | V99V99.A99A99. | 21 | 15 | 18 | 54 | Yes | Yes | Yes |
| | | | | | D99D99.F99F99. | G99G99.S99S99. | F99F99.Y99Y99. | | | | | | | |
| | | | | | G99G99.Q99Q99. | R99R99. | S99S99.K99K99. | | | | | | | |
| | | | | | S92B00. | | | | | | | | | |
| 143 | 12 | SGSVVFA | IRDPE | HVFRPSCYCGYVT | S99S99.G99G99. | I99I99.R99R99. | H91B00.V91B00. | 21 | 15 | 39 | 75 | Yes | Yes | Yes |
| | | | | | S99S99.V99V99. | D99D99.P99P99. | F91B00.R91B00. | | | | | | | |
| | | | | | V99V99.F99F99. | E99E99. | P91B00.S91B00. | | | | | | | |
| | | | | | A99A99. | | C91B00.Y91B00. | | | | | | | |
| | | | | | | | C91B00.G91B00. | | | | | | | |
| | | | | | | | Y91B00.V99V99. | | | | | | | |
| | | | | | | | T91B00. | | | | | | | |
| 193 | 12 | RILKCVF | VGGFY | YRLAEPYEECLME | R99R99.I99I99. | V99V99.G99G99. | B00 | 21 | 15 | 0 | 36 | Yes | Yes | Yes |
| | | | | | L99L99.K91B00. | G99G99.F99F99. | | | | | | | | |
| | | | | | C99C99.V99V99. | Y99Y99. | | | | | | | | |
| | | | | | F99F99. | | | | | | | | | |
| 224 | 12 | HCLEQS | SAQTH | RNWWIE | H99H99.C99C99. | S91B00.A99A99. | R58A41.N58D41. | 18 | 15 | 13 | 46 | Yes | Yes | Yes |
| | | | | | L99L99.E99E99. | Q99Q99.T99T99. | W58S41.W99W99. | | | | | | | |
| | | | | | Q99Q99.S99S99. | H99H99. | I58A41.E58R41. | | | | | | | |
| 238 | 12 | YFSGCVF | FGNNG | AIFKHCSEVS | Y99Y99.F99F99. | F99F99.G99G99. | A99A99.I99I99. | 21 | 15 | 30 | 66 | Yes | Yes | Yes |
| | | | | | S99S99.G99G99. | N99N99.N99N99. | F99F99.K99K99. | | | | | | | |
| | | | | | C99C99.V99V99. | G99G99. | H99H99.C99C99. | | | | | | | |
| | | | | | F99F99. | | S99S99.E99E99. | | | | | | | |
| | | | | | | | V99V99.S99S99. | | | | | | | |
| 268 | 12 | APTGLVY | PMYSL | CLFRNSSVA | A99A99.P99P99. | P99P99.M99M99. | C58A41.L58Y41. | 21 | 15 | 18 | 54 | Yes | Yes | Yes |
| | | | | | T99T99.G99G99. | Y99Y99.S99S99. | F58M41.R58T41. | | | | | | | |
| | | | | | L99L99.V99V99. | L99L99. | N58P41.S58T41. | | | | | | | |
| | | | | | Y99Y99. | | S58G41.V58G41. | | | | | | | |
| | | | | | | | A58V41. | | | | | | | |
| 284 | 12 | VVSLLIW | LSLSR | MTRFGW | V99V99.V99V99. | L99L99.S99S99. | M99M99.T99T99. | 21 | 15 | 18 | 54 | Yes | Yes | Yes |
| | | | | | S99S99.L99L99. | L99L99.S99S99. | R99R99.F99F99. | | | | | | | |
| | | | | | L99L99.I99I99. | R99R99. | G99G99.W99W99. | | | | | | | |
| | | | | | W99W99. | | | | | | | | | |
| 299 | 12 | ALGDRIY | SADSA | AFFNWNDIY | A99A99.L99L99. | S99S99.A99A99. | A99A99.F99F99. | 21 | 15 | 27 | 63 | Yes | Yes | Yes |
| | | | | | D91B00.G99G99. | D99D99.A99A99. | F99F99.N99N99. | | | | | | | |
| | | | | | R99R99.I99I99. | S99S99. | W99W99.N99N99. | | | | | | | |
| | | | | | Y91B00. | | D99D99.I99I99. | | | | | | | |
| | | | | | | | Y99Y99. | | | | | | | |
| 307 | 12 | RVKRLIFLIF | RSGEF | FSLIHHSCCN | R99R99.V99V99. | R99R99.S99S99. | F50I41.S91B00. | 21 | 15 | 15 | 51 | Yes | Yes | Yes |
| | | | | | K99K99.R99R99. | G99G99.E99E99. | L91B00.I50B00. | | | | | | | |
| | | | | | L99L99.I99I99. | F99F99. | H50B00.H58B00. | | | | | | | |
| | | | | | F99F99. | | S50G41.C50B00. | | | | | | | |
| | | | | | | | C50B00.N50B00. | | | | | | | |
| 321 | 12 | ILKCRVF | CHD | AFYHNESQCL | I99I99.L91B00. | C99C99.H99H99. | A99A99.F99F99. | 21 | 9 | 30 | 60 | Yes | Yes | Yes |
| | | | | | K99K99.C99C99. | D99D99. | Y99Y99.H99H99. | | | | | | | |
| | | | | | R99R99.V99V99. | | N99N99.E99E99. | | | | | | | |
| | | | | | F99F99. | | S99S99.Q99Q99. | | | | | | | |
| | | | | | | | C99C99.L99L99. | | | | | | | |
| 326 | 12 | FISSCSS | QAVSV | IRYISCPGGELL | F99F99.I99I99. | Q99Q99.A99A99. | I99I99.R99R99. | 21 | 15 | 39 | 75 | Yes | Yes | Yes |
| | | | | | S99S99.S99S99. | V99V99.S91B00. | Y99Y99.I99I99. | | | | | | | |
| | | | | | C99C99.V99V99. | V99V99. | S99S99.C91B00. | | | | | | | |
| | | | | | F99F99. | | S99S99.P99P99. | | | | | | | |
| | | | | | | | G91B00.G99G99. | | | | | | | |
| | | | | | | | E99E99.L99L99. | | | | | | | |
| | | | | | | | L99L99. | | | | | | | |
| 370 | 12 | GHSGTPS | PSAG | WSTFLG | G99G99.H91B00. | P99P99.S99S99. | W99W99.S99S99. | 21 | 12 | 18 | 51 | Yes | Yes | Yes |
| | | | | | S99S99.G99G99. | A99A99.G99G99. | T99T99.F99F99. | | | | | | | |
| | | | | | T99T99.P99P99. | | L99L99.G99G99. | | | | | | | |
| | | | | | S99S99. | | | | | | | | | |
| 373 | 12 | AVAGHGG | YWSDK | FVSCAD | A99A99.V99V99. | Y99Y99.W99W99. | F99F99.V99V99. | 21 | 15 | 18 | 54 | Yes | Yes | Yes |
| | | | | | A99A99.G99G99. | S99S99.D99D99. | S99S99.C99C99. | | | | | | | |
| | | | | | H99H99.G99G99. | K99K99. | A99A99.D99D99. | | | | | | | |
| | | | | | G99G99. | | | | | | | | | |
| 395 | 12 | FTHEMWR | RSLQF | LYAPH | F99F99.T99T99. | R99R99.S99S99. | L99L99.Y99Y99. | 21 | 15 | 15 | 51 | Yes | Yes | Yes |
| | | | | | H99H99.E99E99. | L99L99.Q99Q99. | A99A99.P99P99. | | | | | | | |
| | | | | | M99M99.W99W99. | F99F99. | H99H99. | | | | | | | |
| | | | | | R99R99. | | | | | | | | | |
| 398 | 12 | LRLRMYG | FHTQA | SVCFSF | L99L99.R99R99. | F99F99.H99H99. | S99S99.V99V99. | 21 | 15 | 18 | 54 | Yes | Yes | Yes |
| | | | | | L99L99.R99R99. | T99T99.Q99Q99. | C99C99.F99F99. | | | | | | | |
| | | | | | M99M99.Y99Y99. | A99A99. | S99S99.F99F99. | | | | | | | |
| | | | | | G99G99. | | | | | | | | | |
| 410 | 12 | RCHGSVQ | DVGAW | LHPIRYYISW | R83B00.C83B00. | D83B00.V99V99. | L99L99.H83B00. | 20 | 15 | 30 | 65 | Yes | Yes | Yes |
| | | | | | H83B00.G83B00. | G99G99.A83B00. | P99P99.I83B00. | | | | | | | |
| | | | | | S83B00.V75B00. | W99W99. | R83B00.Y99Y99. | | | | | | | |
| | | | | | Q83B00. | | Y91B00.I83B00. | | | | | | | |
| | | | | | | | S99S99.W99W99. | | | | | | | |
| 421 | 12 | YLCRCTF | TAASC | AHLNLDSSLL | Y99Y99.L99L99. | T99T99.A99A99. | A99A99.H99H99. | 21 | 15 | 30 | 66 | Yes | Yes | Yes |
| | | | | | C99C99.R99R99. | A99A99.S99S99. | L99L99.N99N99. | | | | | | | |
| | | | | | C99C99.T99T99. | C99C99. | L99L99.D99D99. | | | | | | | |
| | | | | | F99F99. | | S99S99.S99S99. | | | | | | | |
| | | | | | | | L99L99.L99L99. | | | | | | | |
| 437 | 12 | VGHVLSA | FNHAY | TTVAMYCRR | V99V99.G99G99. | F91B00.N99N99. | T99T99.T99T99. | 21 | 15 | 27 | 63 | Yes | Yes | Yes |
| | | | | | H99H99.V99V99. | H99H99.A99A99. | V99V99.A99A99. | | | | | | | |
| | | | | | L99L99.S99S99. | Y99Y99. | M99M99.Y99Y99. | | | | | | | |
| | | | | | A99A99. | | C99C99.R99R99. | | | | | | | |
| | | | | | | | R99R99. | | | | | | | |
| 509 | 12 | VTGSSND | CYGIS | H | V99V99.T99T99. | C99C99.Y99Y99. | H99H99. | 21 | 15 | 3 | 39 | Yes | Yes | Yes |
| | | | | | G99G99.S99S99. | G99G99.I99I99. | | | | | | | | |
| | | | | | S99S99.N99N99. | S99S99. | | | | | | | | |
| | | | | | D99D99. | | | | | | | | | |
| 53 | 12 | PLVCWHD | RVAFE | GHNSALGRS | P99P99.L99L99. | R99R99.V99V99. | G99G99.H99H99. | 21 | 15 | 27 | 63 | Yes | Yes | Yes |
| | | | | | V99V99.C99C99. | A99A99.F99F99. | N99N99.S99S99. | | | | | | | |
| | | | | | W99W99.H99H99. | E99E99. | A99A99.L99L99. | | | | | | | |
| | | | | | D99D99. | | G99G99.R99R99. | | | | | | | |
| | | | | | | | S99S99. | | | | | | | |
| 530 | 12 | YFERIVF | RDWNI | AQFQNNLCLQ | Y99Y99.F99F99. | R66B00.D66B00. | B00B00.B00B00. | 18 | 10 | 15 | 43 | Yes | Yes | Yes |
| | | | | | E66B00.R99R99. | W66B00.N66B00. | L66B00.B00B00. | | | | | | | |
| | | | | | I66B00.V99V99. | I66B00. | H66B00.T66B00. | | | | | | | |
| | | | | | F66B00. | | L66B00.B00B00. | | | | | | | |
| | | | | | | | V66B00.B00B00. | | | | | | | |
| 610 | 12 | CIMSLVW | ESHDC | LISTSSDASY | C99C99.I99I99. | E99E99.S99S99. | L99L99.I91B00. | 21 | 15 | 30 | 66 | Yes | Yes | Yes |
| | | | | | M99M99.S99S99. | H99H99.D99D99. | S99S99.T99T99. | | | | | | | |
| | | | | | L99L99.V99V99. | C99C99. | S99S99.S99S99. | | | | | | | |
| | | | | | W99W99. | | D99D99.A99A99. | | | | | | | |
| | | | | | | | S99S99.Y99Y99. | | | | | | | |
| 626 | 12 | VVALIMS | MLTGH | PTRYMH | V99V99.V99V99. | M99M99.L99L99. | P99P99.T99T99. | 21 | 15 | 18 | 54 | Yes | Yes | Yes |
| | | | | | A99A99.L99L99. | T99T99.G99G99. | R99R99.Y99Y99. | | | | | | | |
| | | | | | I99I99.M99M99. | H99H99. | M99M99.H99H99. | | | | | | | |
| | | | | | S99S99. | | | | | | | | | |
| 632 | 12 | SMKVVLL | PNRGF | ITYYEDLVA | S99S99.M99M99. | P99P99.N99N99. | I99I99.T99T99. | 21 | 15 | 27 | 63 | Yes | Yes | Yes |
| | | | | | K99K99.V99V99. | R99R99.G99G99. | Y99Y99.Y99Y99. | | | | | | | |
| | | | | | V99V99.L99L99. | F99F99. | E99E99.D91B00. | | | | | | | |
| | | | | | L99L99. | | L99L99.V99V99. | | | | | | | |
| | | | | | | | A99A99. | | | | | | | |
| 635 | 12 | GPGYVVW | RAEGV | VIYTGVEVM | G99G99.P99P99. | R99R99.A99A99. | V99V99.I99I99. | 21 | 15 | 27 | 63 | Yes | Yes | Yes |
| | | | | | G99G99.Y99Y99. | E99E99.G99G99. | Y99Y99.T99T99. | | | | | | | |
| | | | | | V99V99.V99V99. | V99V99. | G99G99.V99V99. | | | | | | | |
| | | | | | W99W99. | | E99E99.V99V99. | | | | | | | |
| | | | | | | | M99M99. | | | | | | | |
| 644 | 12 | SCVSVRF | HGQDV | LIYDKSSLGL | S99S99.C99C99. | H99H99.G99G99. | L99L99.I99I99. | 21 | 15 | 30 | 66 | Yes | Yes | Yes |
| | | | | | V99V99.S99S99. | Q99Q99.D99D99. | Y99Y99.D99D99. | | | | | | | |
| | | | | | V99V99.R99R99. | V99V99. | K99K99.S99S99. | | | | | | | |
| | | | | | F99F99. | | S99S99.L99L99. | | | | | | | |
| | | | | | | | G99G99.L99L99. | | | | | | | |
| 656 | 12 | WPCFTRY | AHSMD | PHCTSTNGFVIVS | W99W99.P99P99. | A99A99.H99H99. | P99P99.H99H99. | 21 | 15 | 39 | 75 | Yes | Yes | Yes |
| | | | | | C99C99.F99F99. | S99S99.M99M99. | C99C99.T99T99. | | | | | | | |
| | | | | | T99T99.R99R99. | D99D99. | S99S99.T99T99. | | | | | | | |
| | | | | | Y99Y99. | | N99N99.G99G99. | | | | | | | |
| | | | | | | | F99F99.V99V99. | | | | | | | |
| | | | | | | | I99I99.V99V99. | | | | | | | |
| | | | | | | | S99S99. | | | | | | | |
| 725 | 12 | SLRAKVY | NDFRD | VTFNLN | S99S99.L99L99. | N99N99.D99D99. | B00 | 21 | 15 | 0 | 36 | Yes | Yes | Yes |
| | | | | | R99R99.A99A99. | F99F99.R99R99. | | | | | | | | |
| | | | | | K91B00.V99V99. | D99D99. | | | | | | | | |
| | | | | | Y99Y99. | | | | | | | | | |
| 731 | 12 | CWVGACM | CSGDW | SLINLRLEVV | C99C99.W99W99. | C99C99.S99S99. | S99S99.L99L99. | 21 | 15 | 30 | 66 | Yes | Yes | Yes |
| | | | | | V99V99.G99G99. | G99G99.D99D99. | I99I99.N99N99. | | | | | | | |
| | | | | | A99A99.C99C99. | W99W99. | L99L99.R99R99. | | | | | | | |
| | | | | | M99M99. | | L99L99.E99E99. | | | | | | | |
| | | | | | | | V99V99.V99V99. | | | | | | | |
| 828 | 12 | AARRLVF | MDNVV | VQFNGTAVA | A99A99.A99A99. | M99M99.D99D99. | V99V99.Q99Q99. | 21 | 15 | 27 | 63 | Yes | Yes | Yes |
| | | | | | R99R99.R99R99. | N99N99.V99V99. | F99F99.N99N99. | | | | | | | |
| | | | | | L99L99.V99V99. | V99V99. | G99G99.T99T99. | | | | | | | |
| | | | | | F99F99. | | A99A99.V99V99. | | | | | | | |
| | | | | | | | A99A99. | | | | | | | |
| 879 | 12 | PRDFVRV | VFAGN | YSPCCS | P99P99.R99R99. | V99V99.F99F99. | B00 | 21 | 15 | 0 | 36 | Yes | Yes | Yes |
| | | | | | D99D99.F99F99. | A99A99.G99G99. | | | | | | | | |
| | | | | | V99V99.R99R99. | N99N99. | | | | | | | | |
| | | | | | V99V99. | | | | | | | | | |
| 178 | 11 | SSNCWVL | RVNDV | ISDIGV | S99S99.S99S99. | R99R99.V99V99. | I99I99.S99S99. | 21 | 15 | 18 | 54 | Yes | Yes | Yes |
| | | | | | N99N99.C99C99. | N99N99.D99D99. | D99D99.I99I99. | | | | | | | |
| | | | | | W99W99.V99V99. | V99V99. | G99G99.V99V99. | | | | | | | |
| | | | | | L99L99. | | | | | | | | | |
| 186 | 11 | SVVDPQL | SWPSQ | HCWSGGIRC | S99S99.V99V99. | S99S99.W99W99. | H99H99.C99C99. | 21 | 15 | 27 | 63 | Yes | Yes | Yes |
| | | | | | V99V99.D99D99. | P99P99.S99S99. | W99W99.S99S99. | | | | | | | |
| | | | | | P99P99.Q99Q99. | Q99Q99. | G99G99.G99G99. | | | | | | | |
| | | | | | L99L99. | | I99I99.R99R99. | | | | | | | |
| | | | | | | | C99C99. | | | | | | | |
| 203 | 11 | HSLPGVI | LGGVA | FQCLACEFS | H81B00.S81B00. | L99L99.G99G99. | F90B00.Q99Q99. | 21 | 15 | 27 | 63 | Yes | Yes | Yes |
| | | | | | L81B00.P81B00. | G99G99.V99V99. | C99C99.L99L99. | | | | | | | |
| | | | | | G81B00.V81B00. | A99A99. | A99A99.C99C99. | | | | | | | |
| | | | | | I81B00. | | E99E99.F99F99. | | | | | | | |
| | | | | | | | S99S99. | | | | | | | |
| 208 | 11 | IFNKVVF | PDVSL | AVLNSDALVT | I99I99.F99F99. | P99P99.D99D99. | A99A99.V54Y45. | 21 | 15 | 23 | 59 | Yes | Yes | Yes |
| | | | | | N99N99.K99K99. | V99V99.S99S99. | L54Y45.N54H45. | | | | | | | |
| | | | | | V99V99.V99V99. | L99L99. | S54A45.D54A45. | | | | | | | |
| | | | | | F99F99. | | A54N45.L99L99. | | | | | | | |
| | | | | | | | V90B00.T54L45. | | | | | | | |
| 263 | 11 | PVEFWYW | SMSLA | VSDY | P99P99.V99V99. | S99S99.M99M99. | B00 | 21 | 15 | 0 | 36 | Yes | Yes | Yes |
| | | | | | E99E99.F99F99. | S99S99.L99L99. | | | | | | | | |
| | | | | | W99W99.Y99Y99. | A99A99. | | | | | | | | |
| | | | | | W99W99. | | | | | | | | | |
| 334 | 11 | FEQFDWR | SAYST | GTFLEDYFYR | F99F99.E99E99. | S99S99.A99A99. | G99G99.T99T99. | 21 | 15 | 30 | 66 | Yes | Yes | Yes |
| | | | | | Q99Q99.F99F99. | Y99Y99.S99S99. | F99F99.L99L99. | | | | | | | |
| | | | | | D99D99.W99W99. | T99T99. | E99E99.D99D99. | | | | | | | |
| | | | | | R99R99. | | Y99Y99.F99F99. | | | | | | | |
| | | | | | | | Y99Y99.R99R99. | | | | | | | |
| 386 | 11 | FASLSDLCF | CLGW | YSHDLNSVVR | F99F99.A90B00. | C99C99.L99L99. | Y99Y99.S81B00. | 21 | 12 | 30 | 63 | Yes | Yes | Yes |
| | | | | | S99S99.D99D99. | G99G99.W99W99. | H81B00.D81B00. | | | | | | | |
| | | | | | L99L99.C99C99. | | L81B00.N81B00. | | | | | | | |
| | | | | | F99F99. | | S81B00.V99V99. | | | | | | | |
| | | | | | | | V81B00.R81B00. | | | | | | | |
| 391 | 11 | ALSCGVF | GLGVD | AFFSSTGTVE | A99A99.L99L99. | G99G99.L99L99. | A99A99.F99F99. | 21 | 15 | 30 | 66 | Yes | Yes | Yes |
| | | | | | S99S99.C99C99. | G99G99.V99V99. | F99F99.S90B00. | | | | | | | |
| | | | | | G99G99.V99V99. | D99D99. | S99S99.T90B00. | | | | | | | |
| | | | | | F99F99. | | G90B00.T90B00. | | | | | | | |
| | | | | | | | V90B00.E90B00. | | | | | | | |
| 409 | 11 | DGRRVVY | VDGYC | AAYLAGGVS | D99D99.G99G99. | V99V99.D99D99. | A99A99.A99A99. | 21 | 15 | 27 | 63 | Yes | Yes | Yes |
| | | | | | R99R99.R99R99. | G99G99.Y99Y99. | Y99Y99.L99L99. | | | | | | | |
| | | | | | V99V99.V99V99. | C99C99. | A99A99.G99G99. | | | | | | | |
| | | | | | Y99Y99. | | G99G99.V99V99. | | | | | | | |
| | | | | | | | S99S99. | | | | | | | |
| 426 | 11 | RADIYWM | WRTSD | VYLAGGVGY | R99R99.A99A99. | W99W99.R99R99. | V99V99.Y99Y99. | 21 | 15 | 27 | 63 | Yes | Yes | Yes |
| | | | | | D90B00.I99I99. | T99T99.S99S99. | L99L99.A99A99. | | | | | | | |
| | | | | | Y99Y99.W99W99. | D90B00. | G99G99.G99G99. | | | | | | | |
| | | | | | M99M99. | | V99V99.G99G99. | | | | | | | |
| | | | | | | | Y99Y99. | | | | | | | |
| 428 | 11 | HARCSVI | LAESV | VLPAMRLRY | H99H99.A99A99. | L99L99.A99A99. | V99V99.L99L99. | 21 | 15 | 27 | 63 | Yes | Yes | Yes |
| | | | | | R99R99.C99C99. | E99E99.S99S99. | P99P99.A99A99. | | | | | | | |
| | | | | | S99S99.V99V99. | V99V99. | M99M99.R99R99. | | | | | | | |
| | | | | | I90B00. | | L99L99.R99R99. | | | | | | | |
| | | | | | | | Y99Y99. | | | | | | | |
| 443 | 11 | GLHLALT | QVLKV | QILSHFDIVS | G99G99.L99L99. | Q99Q99.V99V99. | Q99Q99.I99I99. | 21 | 15 | 30 | 66 | Yes | Yes | Yes |
| | | | | | H99H99.L99L99. | L99L99.K99K99. | L99L99.S99S99. | | | | | | | |
| | | | | | A99A99.L90B00. | V99V99. | H99H99.F99F99. | | | | | | | |
| | | | | | T90B00. | | D99D99.I99I99. | | | | | | | |
| | | | | | | | V99V99.S99S99. | | | | | | | |
| 499 | 11 | NDLRCLI | MIGNT | VDGFW | N99N99.D99D99. | M99M99.I99I99. | V99V99.D99D99. | 21 | 15 | 15 | 51 | Yes | Yes | Yes |
| | | | | | L99L99.R99R99. | G99G99.N99N99. | G99G99.F99F99. | | | | | | | |
| | | | | | C99C99.L99L99. | T99T99. | W99W99. | | | | | | | |
| | | | | | I99I99. | | | | | | | | | |
| 523 | 11 | KWLVY | YHHAG | AFWTTGDRVE | K99K99.W99W99. | Y99Y99.H99H99. | A99A99.F99F99. | 15 | 15 | 30 | 60 | Yes | Yes | Yes |
| | | | | | L99L99.V99V99. | H99H99.A99A99. | W99W99.T99T99. | | | | | | | |
| | | | | | Y99Y99. | G99G99. | T99T99.G99G99. | | | | | | | |
| | | | | | | | D99D99.R99R99. | | | | | | | |
| | | | | | | | V99V99.E99E99. | | | | | | | |
| 525 | 11 | GHTCGSR | FRPPH | LVVPYY | G99G99.H90B00. | F99F99.R99R99. | L99L99.V99V99. | 21 | 15 | 18 | 54 | Yes | Yes | Yes |
| | | | | | T99T99.C99C99. | P99P99.P99P99. | V99V99.P99P99. | | | | | | | |
| | | | | | G99G99.S99S99. | H99H99. | Y99Y99.Y99Y99. | | | | | | | |
| | | | | | R99R99. | | | | | | | | | |
| 536 | 11 | DYLYVYA | RMSDS | VINIRC | D99D99.Y99Y99. | R99R99.M99M99. | V99V99.I99I99. | 21 | 15 | 18 | 54 | Yes | Yes | Yes |
| | | | | | L99L99.Y99Y99. | S99S99.D99D99. | N99N99.I99I99. | | | | | | | |
| | | | | | V99V99.Y99Y99. | S99S99. | R99R99.C99C99. | | | | | | | |
| | | | | | A99A99. | | | | | | | | | |
| 539 | 11 | ICCCYET | ATRPE | NIRGFS | I99I99.C99C99. | A99A99.T99T99. | B00 | 21 | 15 | 0 | 36 | Yes | Yes | Yes |
| | | | | | C99C99.C99C99. | R99R99.P99P99. | | | | | | | | |
| | | | | | Y99Y99.E99E99. | E99E99. | | | | | | | | |
| | | | | | T99T99. | | | | | | | | | |
| 593 | 11 | VVGVVLG | LSHES | YSRFLH | V99V99.V99V99. | L99L99.S99S99. | Y99Y99.S99S99. | 21 | 15 | 18 | 54 | Yes | Yes | Yes |
| | | | | | G99G99.V99V99. | H99H99.E99E99. | R99R99.F99F99. | | | | | | | |
| | | | | | V99V99.L99L99. | S99S99. | L99L99.H99H99. | | | | | | | |
| | | | | | G99G99. | | | | | | | | | |
| 598 | 11 | TRTVNVC | GWDEI | HVLPLVGLGVRA | T99T99.R99R99. | G99G99.W99W99. | H99H99.V99V99. | 21 | 15 | 39 | 75 | Yes | Yes | Yes |
| | | | | | T99T99.V99V99. | D99D99.E99E99. | L99L99.I99I99. | | | | | | | |
| | | | | | N99N99.V99V99. | I99I99. | P99P99.L99L99. | | | | | | | |
| | | | | | C99C99. | | V99V99.G99G99. | | | | | | | |
| | | | | | | | L99L99.G99G99. | | | | | | | |
| | | | | | | | V99V99.R99R99. | | | | | | | |
| | | | | | | | A99A99. | | | | | | | |
| 655 | 11 | R | PHNLG | IEPAVEFFKF | B00 | P99P99.H99H99. | I99I99.E99E99. | 0 | 15 | 30 | 45 | Yes | Yes | Yes |
| | | | | | | N99N99.L99L99. | P99P99.A99A99. | | | | | | | |
| | | | | | | G99G99. | V99V99.E99E99. | | | | | | | |
| | | | | | | | F99F99.F99F99. | | | | | | | |
| | | | | | | | K99K99.F99F99. | | | | | | | |
| 690 | 11 | WR | MSCVC | LRIGENCEVH | W99W99.R99R99. | M99M99.S99S99. | L99L99.R99R99. | 6 | 15 | 30 | 51 | Yes | Yes | Yes |
| | | | | | | C99C99.V99V99. | I99I99.G99G99. | | | | | | | |
| | | | | | | C90B00. | E99E99.N99N99. | | | | | | | |
| | | | | | | | C99C99.E99E99. | | | | | | | |
| | | | | | | | V99V99.H99H99. | | | | | | | |
| 695 | 11 | FHVGSNV | GYVGD | LATRPNCPSN | F99F99.H99H99. | G99G99.Y99Y99. | L90B00.A90B00. | 21 | 15 | 30 | 66 | Yes | Yes | Yes |
| | | | | | V99V99.G99G99. | V99V99.G99G99. | T90B00.R90B00. | | | | | | | |
| | | | | | S99S99.N90B00. | D99D99. | P90B00.N90B00. | | | | | | | |
| | | | | | V99V99. | | C90B00.P90B00. | | | | | | | |
| | | | | | | | S90B00.N90B00. | | | | | | | |
| 710 | 11 | STSYYTY | HCFNS | ISGDFGY | S90B00.T99T99. | H99H99.C99C99. | I99I99.S99S99. | 21 | 15 | 21 | 57 | Yes | Yes | Yes |
| | | | | | S99S99.Y99Y99. | F99F99.N99N99. | G99G99.D99D99. | | | | | | | |
| | | | | | Y99Y99.T99T99. | S99S99. | F99F99.G99G99. | | | | | | | |
| | | | | | Y99Y99. | | Y99Y99. | | | | | | | |
| 714 | 11 | RCHRRVY | SPGSG | ATMADDHLVS | R99R99.C99C99. | S99S99.P99P99. | A99A99.T99T99. | 21 | 15 | 30 | 66 | Yes | Yes | Yes |
| | | | | | H99H99.R99R99. | G99G99.S99S99. | M99M99.A99A99. | | | | | | | |
| | | | | | R99R99.V90B00. | G99G99. | D99D99.D99D99. | | | | | | | |
| | | | | | Y99Y99. | | H99H99.L99L99. | | | | | | | |
| | | | | | | | V99V99.S99S99. | | | | | | | |
| 727 | 11 | GQIPALV | RNFLF | ICEHY | G99G99.Q99Q99. | R99R99.N99N99. | I99I99.C99C99. | 21 | 15 | 15 | 51 | Yes | Yes | Yes |
| | | | | | I99I99.P99P99. | F99F99.L99L99. | E99E99.H99H99. | | | | | | | |
| | | | | | A99A99.L99L99. | F99F99. | Y99Y99. | | | | | | | |
| | | | | | V99V99. | | | | | | | | | |
| 735 | 11 | GFCYADR | SSG | QYYLGPNYFF | G99G99.F99F99. | S99S99.S99S99. | Q99Q99.Y99Y99. | 21 | 15 | 30 | 66 | Yes | Yes | Yes |
| | | | | | C99C99.Y99Y99. | G99G99.L99L99. | Y99Y99.L99L99. | | | | | | | |
| | | | | | A99A99.D99D99. | C99C99. | G99G99.P99P99. | | | | | | | |
| | | | | | R99R99. | | N99N99.Y99Y99. | | | | | | | |
| | | | | | | | F99F99.F99F99. | | | | | | | |
| 739 | 11 | PAYYFSR | YFSTL | LSITPDTCYW | P99P99.A99A99. | Y99Y99.F99F99. | L99L99.S99S99. | 21 | 15 | 30 | 66 | Yes | Yes | Yes |
| | | | | | Y99Y99.Y99Y99. | S99S99.T99T99. | I99I99.T99T99. | | | | | | | |
| | | | | | F99F99.S99S99. | L99L99. | P99P99.D99D99. | | | | | | | |
| | | | | | R99R99. | | T99T99.C99C99. | | | | | | | |
| | | | | | | | Y99Y99.W99W99. | | | | | | | |
| 740 | 11 | HQVVH | RDDTA | HYLHRSQCVM | H99H99.Q99Q99. | R99R99.D99D99. | H99H99.Y99Y99. | 21 | 15 | 30 | 66 | Yes | Yes | Yes |
| | | | | | D99D99.C99C99. | D99D99.T99T99. | L99L99.H99H99. | | | | | | | |
| | | | | | V99V99.V99V99. | A99A99. | R99R99.S99S99. | | | | | | | |
| | | | | | H99H99. | | Q99Q99.C99C99. | | | | | | | |
| | | | | | | | V99V99.M99M99. | | | | | | | |
| 760 | 11 | LTLLPFC | LTREA | FCRRVVDGLWLRY | L99L99.T99T99. | L99L99.T99T99. | F99F99.C99C99. | 21 | 15 | 39 | 75 | Yes | Yes | Yes |
| | | | | | L99L99.L99L99. | R99R99.E99E99. | R99R99.R99R99. | | | | | | | |
| | | | | | P99P99.F99F99. | A99A99. | V99V99.V99V99. | | | | | | | |
| | | | | | C99C99. | | D99D99.G99G99. | | | | | | | |
| | | | | | | | L99L99.W99W99. | | | | | | | |
| | | | | | | | L99L99.R99R99. | | | | | | | |
| | | | | | | | Y99Y99. | | | | | | | |
| 762 | 11 | KIFESFR | SKAGI | LTIELDNSLI | K99K99.I99I99. | S99S99.K99K99. | L99L99.T99T99. | 21 | 15 | 30 | 66 | Yes | Yes | Yes |
| | | | | | F99F99.E99E99. | A99A99.G99G99. | I99I99.E99E99. | | | | | | | |
| | | | | | S99S99.F99F99. | I99I99. | L99L99.D99D99. | | | | | | | |
| | | | | | R99R99. | | N99N99.S99S99. | | | | | | | |
| | | | | | | | L99L99.I99I99. | | | | | | | |
| 775 | 11 | ATCRVIR | TSCEA | LALFRGPCML | A99A99.T90B00. | T99T99.S99S99. | L99L99.A99A99. | 21 | 15 | 30 | 66 | Yes | Yes | Yes |
| | | | | | C99C99.R99R99. | C90B00.E99E99. | L99L99.F99F99. | | | | | | | |
| | | | | | V99V99.I99I99. | A99A99. | R99R99.G99G99. | | | | | | | |
| | | | | | R99R99. | | P99P99.C99C99. | | | | | | | |
| | | | | | | | M99M99.L99L99. | | | | | | | |
| 788 | 11 | IRRSYST | DRGYR | ALIDSEGYLA | I90B00.R90B00. | D99D99.R99R99. | A99A99.L99L99. | 21 | 15 | 30 | 66 | Yes | Yes | Yes |
| | | | | | R99R99.S99S99. | G99G99.Y99Y99. | I99I99.D99D99. | | | | | | | |
| | | | | | Y99Y99.S99S99. | R99R99. | S99S99.E99E99. | | | | | | | |
| | | | | | T99T99. | | G99G99.Y99Y99. | | | | | | | |
| | | | | | | | L99L99.A99A99. | | | | | | | |
| 795 | 11 | KNVIINV | ADGWF | IYYSVSKSNNS | K99K99.N90B00. | A99A99.D99D99. | I99I99.Y99Y99. | 21 | 15 | 33 | 69 | Yes | Yes | Yes |
| | | | | | V99V99.I99I99. | G99G99.W99W99. | Y99Y99.S99S99. | | | | | | | |
| | | | | | I99I99.N90B00. | F99F99. | V99V99.S99S99. | | | | | | | |
| | | | | | V99V99. | | K99K99.S99S99. | | | | | | | |
| | | | | | | | N99N99.N99N99. | | | | | | | |
| | | | | | | | S99S99. | | | | | | | |
| 821 | 11 | SARWVVY | TGYPG | VCFSQILMHTATV | S99S99.A99A99. | T99T99.G99G99. | V99V99.C99C99. | 21 | 15 | 39 | 75 | Yes | Yes | Yes |
| | | | | | R99R99.W99W99. | Y99Y99.P99P99. | F99F99.S99S99. | | | | | | | |
| | | | | | V99V99.V99V99. | G99G99. | Q99Q99.I99I99. | | | | | | | |
| | | | | | Y90B00. | | L99L99.M99M99. | | | | | | | |
| | | | | | | | H99H99.T99T99. | | | | | | | |
| | | | | | | | A99A99.T99T99. | | | | | | | |
| | | | | | | | V99V99. | | | | | | | |
| 837 | 11 | GSVRIVY | GSCLT | SACCDY | G99G99.S99S99. | G99G99.S99S99. | S99S99.A99A99. | 21 | 15 | 18 | 54 | Yes | Yes | Yes |
| | | | | | V99V99.R99R99. | C99C99.L99L99. | C99C99.C99C99. | | | | | | | |
| | | | | | I99I99.V99V99. | T99T99. | D99D99.Y99Y99. | | | | | | | |
| | | | | | Y99Y99. | | | | | | | | | |
| 864 | 11 | TVVKVHW | YSTLL | LIEGSHSGFQSTY | T90B00.V99V99. | Y99Y99.S99S99. | L99L99.I99I99. | 21 | 15 | 39 | 75 | Yes | Yes | Yes |
| | | | | | V99V99.K99K99. | T99T99.L99L99. | E99E99.G99G99. | | | | | | | |
| | | | | | V99V99.H99H99. | L99L99. | S99S99.H99H99. | | | | | | | |
| | | | | | W99W99. | | S99S99.G99G99. | | | | | | | |
| | | | | | | | F99F99.Q99Q99. | | | | | | | |
| | | | | | | | S99S99.T99T99. | | | | | | | |
| | | | | | | | Y99Y99. | | | | | | | |
| 867 | 11 | MSSWCTW | PIGTK | IDDWGC | M99M99.S99S99. | P99P99.I99I99. | I99I99.D99D99. | 21 | 15 | 18 | 54 | Yes | Yes | Yes |
| | | | | | S99S99.W99W99. | G99G99.T99T99. | D99D99.W99W99. | | | | | | | |
| | | | | | C99C99.T99T99. | K99K99. | G99G99.C99C99. | | | | | | | |
| | | | | | W99W99. | | | | | | | | | |
| 907 | 11 | KFVYCLA | WHHAA | HYSLDTDEFMEVA | B00 | W90B00.H90B00. | H90B00.Y90B00. | 0 | 15 | 39 | 54 | Yes | Yes | Yes |
| | | | | | | H90B00.A99A99. | S90B00.L90B00. | | | | | | | |
| | | | | | | A90B00. | D90B00.T90B00. | | | | | | | |
| | | | | | | | D90B00.E90B00. | | | | | | | |
| | | | | | | | F90B00.M90B00. | | | | | | | |
| | | | | | | | E90B00.V90B00. | | | | | | | |
| | | | | | | | A90B00. | | | | | | | |
| 99 | 11 | VIERLVY | RDGHS | ITVDHLISVL | V99V99.I99I99. | R99R99.D99D99. | I90B00.T90B00. | 21 | 15 | 30 | 66 | Yes | Yes | Yes |
| | | | | | E99E99.R99R99. | G99G99.H99H99. | V90B00.D90B00. | | | | | | | |
| | | | | | L99L99.V99V99. | S99S99. | H90B00.L90B00. | | | | | | | |
| | | | | | Y99Y99. | | I90B00.S90B00. | | | | | | | |
| | | | | | | | V90B00.L90B00. | | | | | | | |
| 110 | 10 | FDFAGDVA | SWWCV | YITPVS | F99F99.D80B00. | S99S99.W99W99. | Y90B00.I90B00. | 23 | 15 | 18 | 56 | Yes | Yes | Yes |
| | | | | | F99F99.A99A99. | W99W99.C99C99. | T90B00.P99P99. | | | | | | | |
| | | | | | G99G99.D99D99. | V99V99. | V90B00.S90B00. | | | | | | | |
| | | | | | V99V99.A99A99. | | | | | | | | | |
| 151 | 10 | DTLCNSI | TSPSC | FFFYHRCHRC | D99D99.T99T99. | T99T99.S99S99. | F99F99.F99F99. | 21 | 15 | 30 | 66 | Yes | Yes | Yes |
| | | | | | L99L99.C99C99. | P99P99.S99S99. | F99F99.Y99Y99. | | | | | | | |
| | | | | | N99N99.S99S99. | C99C99. | H99H99.R99R99. | | | | | | | |
| | | | | | I99I99. | | C99C99.H99H99. | | | | | | | |
| | | | | | | | R99R99.C99C99. | | | | | | | |
| 205 | 10 | SDVPGPY | PG | IDDYGW | S90B00.D99D99. | P99P99.G99G99. | I99I99.D90B00. | 21 | 6 | 18 | 45 | Yes | Yes | Yes |
| | | | | | V99V99.P99P99. | | D99D99.Y99Y99. | | | | | | | |
| | | | | | G99G99.P99P99. | | G99G99.W99W99. | | | | | | | |
| | | | | | Y99Y99. | | | | | | | | | |
| 230 | 10 | SCSKRVF | YSW | WLYFAPGLLM | S90B00.C90B00. | Y90B00.S90B00. | W99W99.L99L99. | 21 | 9 | 30 | 60 | Yes | Yes | Yes |
| | | | | | S90B00.K90B00. | W90B00. | Y99Y99.F99F99. | | | | | | | |
| | | | | | R90B00.V99V99. | | A99A99.P99P99. | | | | | | | |
| | | | | | F99F99. | | G99G99.L99L99. | | | | | | | |
| | | | | | | | L99L99.M99M99. | | | | | | | |
| 260 | 10 | NSLHVFA | HGSFM | SCFHWGMVR | N99N99.S99S99. | H99H99.G99G99. | S99S99.C99C99. | 21 | 15 | 27 | 63 | Yes | Yes | Yes |
| | | | | | L99L99.H99H99. | S99S99.F99F99. | F99F99.H99H99. | | | | | | | |
| | | | | | V99V99.F99F99. | M99M99. | W99W99.G99G99. | | | | | | | |
| | | | | | A99A99. | | M90B00.V99V99. | | | | | | | |
| | | | | | | | R99R99. | | | | | | | |
| 261 | 10 | YLPRVDD | NSLIC | H | Y99Y99.L99L99. | N99N99.S99S99. | H99H99. | 21 | 15 | 3 | 39 | Yes | Yes | Yes |
| | | | | | P99P99.R99R99. | L99L99.I99I99. | | | | | | | | |
| | | | | | V99V99.D99D99. | C99C99. | | | | | | | | |
| | | | | | D99D99. | | | | | | | | | |
| 280 | 10 | SFAFSLC | RWGA | TLLLSNSRSD | S99S99.F99F99. | R99R99.W99W99. | T99T99.L99L99. | 21 | 12 | 30 | 63 | Yes | Yes | Yes |
| | | | | | A99A99.F99F99. | G99G99.A99A99. | L99L99.L99L99. | | | | | | | |
| | | | | | S99S99.L99L99. | | S99S99.N99N99. | | | | | | | |
| | | | | | C99C99. | | S99S99.R99R99. | | | | | | | |
| | | | | | | | S99S99.D99D99. | | | | | | | |
| 330 | 10 | CFPYDFW | IETGD | PICLG | C99C99.F99F99. | I99I99.E99E99. | P99P99.I99I99. | 21 | 15 | 15 | 51 | Yes | Yes | Yes |
| | | | | | P99P99.Y99Y99. | T99T99.G99G99. | C99C99.L99L99. | | | | | | | |
| | | | | | D99D99.F99F99. | D99D99. | G99G99. | | | | | | | |
| | | | | | W99W99. | | | | | | | | | |
| 374 | 10 | FACHWCA | VESGG | FVPSKVCCGK | F99F99.A99A99. | V99V99.E99E99. | F90B00.V90B00. | 21 | 15 | 30 | 66 | Yes | Yes | Yes |
| | | | | | C99C99.H99H99. | S99S99.G99G99. | P90B00.S90B00. | | | | | | | |
| | | | | | W99W99.C99C99. | G99G99. | K90B00.V90B00. | | | | | | | |
| | | | | | A99A99. | | C90B00.C99C99. | | | | | | | |
| | | | | | | | G90B00.K90B00. | | | | | | | |
| 381 | 10 | RTSRIVG | NFSQC | HCIMSGFGMPWVV | R99R99.T99T99. | N99N99.F99F99. | H99H99.C99C99. | 21 | 15 | 39 | 75 | Yes | Yes | Yes |
| | | | | | S99S99.R99R99. | S99S99.Q99Q99. | I99I99.M99M99. | | | | | | | |
| | | | | | I99I99.V99V99. | C99C99. | S99S99.G99G99. | | | | | | | |
| | | | | | G99G99. | | F99F99.G99G99. | | | | | | | |
| | | | | | | | M99M99.P99P99. | | | | | | | |
| | | | | | | | W99W99.V99V99. | | | | | | | |
| | | | | | | | V99V99. | | | | | | | |
| 418 | 10 | VASADVL | SVSWF | MIRRLG | V99V99.A99A99. | S99S99.V99V99. | B00 | 21 | 15 | 0 | 36 | Yes | Yes | Yes |
| | | | | | S99S99.A99A99. | S99S99.W99W99. | | | | | | | | |
| | | | | | D99D99.V99V99. | F99F99. | | | | | | | | |
| | | | | | L99L99. | | | | | | | | | |
| 427 | 10 | YYHNIVY | GAAEV | ASFSSHCLVA | Y99Y99.Y99Y99. | G99G99.A99A99. | A99A99.S99S99. | 21 | 15 | 30 | 66 | Yes | Yes | Yes |
| | | | | | H99H99.N99N99. | A99A99.E99E99. | F99F99.S99S99. | | | | | | | |
| | | | | | I99I99.V99V99. | V99V99. | S99S99.H99H99. | | | | | | | |
| | | | | | Y99Y99. | | C99C99.L99L99. | | | | | | | |
| | | | | | | | V99V99.A99A99. | | | | | | | |
| 439 | 10 | SVSAVFQ | CLNYV | LASHLKNEVH | S99S99.V99V99. | C99C99.L99L99. | L99L99.A99A99. | 21 | 15 | 30 | 66 | Yes | Yes | Yes |
| | | | | | S99S99.A99A99. | N99N99.Y99Y99. | S99S99.H99H99. | | | | | | | |
| | | | | | V99V99.F99F99. | V99V99. | L99L99.K99K99. | | | | | | | |
| | | | | | Q99Q99. | | N99N99.E99E99. | | | | | | | |
| | | | | | | | V99V99.H99H99. | | | | | | | |
| 475 | 10 | CSLRVVY | CDGTR | ASVTQNLTVL | C99C99.S99S99. | C99C99.D99D99. | A99A99.S99S99. | 21 | 15 | 30 | 66 | Yes | Yes | Yes |
| | | | | | L99L99.R99R99. | G99G99.T99T99. | V99V99.T99T99. | | | | | | | |
| | | | | | V99V99.V99V99. | R99R99. | Q99Q99.N99N99. | | | | | | | |
| | | | | | Y99Y99. | | L99L99.T99T99. | | | | | | | |
| | | | | | | | V99V99.L99L99. | | | | | | | |
| 490 | 10 | VVGILAS | SSGG | NSRWIH | V99V99.V99V99. | S99S99.S99S99. | N99N99.S99S99. | 21 | 12 | 18 | 51 | Yes | Yes | Yes |
| | | | | | G99G99.I99I99. | G99G99.G99G99. | R99R99.W99W99. | | | | | | | |
| | | | | | L99L99.A99A99. | | I99I99.H99H99. | | | | | | | |
| | | | | | S99S99. | | | | | | | | | |
| 491 | 10 | GSYLVYR | CGVLS | FILQTDGMFF | G99G99.S99S99. | C99C99.G99G99. | F99F99.I99I99. | 21 | 15 | 30 | 66 | Yes | Yes | Yes |
| | | | | | Y99Y99.L99L99. | V99V99.L99L99. | L99L99.Q99Q99. | | | | | | | |
| | | | | | V99V99.Y99Y99. | S99S99. | T99T99.D99D99. | | | | | | | |
| | | | | | R99R99. | | G99G99.M99M99. | | | | | | | |
| | | | | | | | F99F99.F99F99. | | | | | | | |
| 519 | 10 | SHVTNEF | LSALC | IDDFGH | S99S99.H99H99. | L99L99.S99S99. | I99I99.D99D99. | 21 | 15 | 18 | 54 | Yes | Yes | Yes |
| | | | | | V99V99.T90B00. | A99A99.L99L99. | D99D99.F99F99. | | | | | | | |
| | | | | | N99N99.E99E99. | C99C99. | G99G99.H99H99. | | | | | | | |
| | | | | | F99F99. | | | | | | | | | |
| 616 | 10 | QAAVCFA | VQYGT | NADPTLLGI | Q99Q99.A99A99. | V99V99.Q99Q99. | N99N99.A99A99. | 21 | 15 | 27 | 63 | Yes | Yes | Yes |
| | | | | | A99A99.V99V99. | Y99Y99.G99G99. | D99D99.P99P99. | | | | | | | |
| | | | | | C99C99.F99F99. | T90B00. | T99T99.L99L99. | | | | | | | |
| | | | | | A99A99. | | L99L99.G99G99. | | | | | | | |
| | | | | | | | I99I99. | | | | | | | |
| 627 | 10 | VNTEFVC | LSVML | ETAYAVNGP | V99V99.N99N99. | L99L99.S99S99. | E99E99.T99T99. | 21 | 15 | 27 | 63 | Yes | Yes | Yes |
| | | | | | T99T99.E99E99. | V99V99.M99M99. | A99A99.Y99Y99. | | | | | | | |
| | | | | | F99F99.V99V99. | L99L99. | A99A99.V90B00. | | | | | | | |
| | | | | | C99C99. | | N99N99.G99G99. | | | | | | | |
| | | | | | | | P99P99. | | | | | | | |
| 631 | 10 | FHRVTG | RHNVD | HVFDSL | F99F99.H99H99. | R99R99.H99H99. | H99H99.V99V99. | 18 | 15 | 18 | 51 | Yes | Yes | Yes |
| | | | | | R99R99.V99V99. | N99N99.V99V99. | F99F99.D90B00. | | | | | | | |
| | | | | | T99T99.G99G99. | D99D99. | S99S99.L99L99. | | | | | | | |
| 671 | 10 | GLRAIMC | SYESR | LVLARDCRLV | G99G99.L99L99. | S99S99.Y99Y99. | L99L99.V99V99. | 21 | 15 | 30 | 66 | Yes | Yes | Yes |
| | | | | | R99R99.A99A99. | E99E99.S99S99. | L99L99.A99A99. | | | | | | | |
| | | | | | I99I99.M90B00. | R99R99. | R99R99.D99D99. | | | | | | | |
| | | | | | C99C99. | | C99C99.R99R99. | | | | | | | |
| | | | | | | | L99L99.V99V99. | | | | | | | |
| 677 | 10 | CRMCAFT | HCALR | FPAATESQFY | C99C99.R90B00. | H90B00.C99C99. | F99F99.P99P99. | 21 | 15 | 30 | 66 | Yes | Yes | Yes |
| | | | | | M99M99.C99C99. | A99A99.L99L99. | A99A99.A99A99. | | | | | | | |
| | | | | | A99A99.F90B00. | R99R99. | T99T99.E99E99. | | | | | | | |
| | | | | | T99T99. | | S99S99.Q99Q99. | | | | | | | |
| | | | | | | | F99F99.Y99Y99. | | | | | | | |
| 697 | 10 | VPIDYVM | TIANV | HNPGVLGHWW | V99V99.P90B00. | T99T99.I99I99. | H99H99.N99N99. | 21 | 15 | 30 | 66 | Yes | Yes | Yes |
| | | | | | I99I99.D99D99. | A99A99.N99N99. | P99P99.G99G99. | | | | | | | |
| | | | | | Y99Y99.V99V99. | V99V99. | V99V99.L99L99. | | | | | | | |
| | | | | | M99M99. | | G99G99.H99H99. | | | | | | | |
| | | | | | | | W99W99.W99W99. | | | | | | | |
| 719 | 10 | VSHHVFG | LGCSL | HYYCDMDVS | V99V99.S99S99. | L99L99.G99G99. | H99H99.Y99Y99. | 21 | 15 | 27 | 63 | Yes | Yes | Yes |
| | | | | | H99H99.H99H99. | C99C99.S99S99. | Y99Y99.C99C99. | | | | | | | |
| | | | | | V99V99.F99F99. | L99L99. | D99D99.M99M99. | | | | | | | |
| | | | | | G99G99. | | D99D99.V99V99. | | | | | | | |
| | | | | | | | S99S99. | | | | | | | |
| 750 | 10 | TIVKSIA | FSDKF | CILAALSFET | T99T99.I99I99. | F99F99.S99S99. | C99C99.I99I99. | 21 | 15 | 30 | 66 | Yes | Yes | Yes |
| | | | | | V99V99.K99K99. | D99D99.K99K99. | L99L99.A99A99. | | | | | | | |
| | | | | | S99S99.I99I99. | F99F99. | A99A99.L99L99. | | | | | | | |
| | | | | | A99A99. | | S99S99.F99F99. | | | | | | | |
| | | | | | | | E99E99.T99T99. | | | | | | | |
| 751 | 10 | DAFIVSH | RCCQ | YNLVL | D99D99.A99A99. | R99R99.C99C99. | Y99Y99.N99N99. | 21 | 12 | 15 | 48 | Yes | Yes | Yes |
| | | | | | F99F99.I99I99. | C99C99.Q99Q99. | L99L99.V99V99. | | | | | | | |
| | | | | | V99V99.S99S99. | | L90B00. | | | | | | | |
| | | | | | H99H99. | | | | | | | | | |
| 777 | 10 | CLVSYEC | ALRNT | LRSFLTVSVSSLV | C99C99.L99L99. | A99A99.L99L99. | B00 | 21 | 15 | 0 | 36 | Yes | Yes | Yes |
| | | | | | V99V99.S99S99. | R99R99.N99N99. | | | | | | | | |
| | | | | | Y90B00.E90B00. | T99T99. | | | | | | | | |
| | | | | | C90B00. | | | | | | | | | |
| 789 | 10 | YFVSIVW | DRGDT | VTWKYSHCVA | Y99Y99.F99F99. | D99D99.R99R99. | V99V99.T90B00. | 21 | 15 | 30 | 66 | Yes | Yes | Yes |
| | | | | | V99V99.S99S99. | G99G99.D99D99. | W99W99.K90B00. | | | | | | | |
| | | | | | I99I99.V99V99. | T99T99. | Y90B00.S90B00. | | | | | | | |
| | | | | | W99W99. | | H90B00.C90B00. | | | | | | | |
| | | | | | | | V90B00.A90B00. | | | | | | | |
| 814 | 10 | VVGYPIA | GRWGG | WCLSDIDTCL | V99V99.V99V99. | G99G99.R99R99. | W99W99.C99C99. | 21 | 15 | 30 | 66 | Yes | Yes | Yes |
| | | | | | G99G99.Y99Y99. | W99W99.G99G99. | L99L99.S99S99. | | | | | | | |
| | | | | | P99P99.I99I99. | G99G99. | D99D99.I99I99. | | | | | | | |
| | | | | | A99A99. | | D99D99.T99T99. | | | | | | | |
| | | | | | | | C99C99.L99L99. | | | | | | | |
| 838 | 10 | IIGCSVY | PDSIT | HLLSRTLNVS | I99I99.I99I99. | P99P99.D99D99. | H99H99.L99L99. | 21 | 15 | 30 | 66 | Yes | Yes | Yes |
| | | | | | G99G99.C99C99. | S99S99.I99I99. | L99L99.S99S99. | | | | | | | |
| | | | | | S99S99.V99V99. | T99T99. | R99R99.T99T99. | | | | | | | |
| | | | | | Y99Y99. | | L99L99.N99N99. | | | | | | | |
| | | | | | | | V99V99.S99S99. | | | | | | | |
| 849 | 10 | ISKRAWS | MGFCH | VALLGNCIC | I99I99.S99S99. | M99M99.G99G99. | V90B00.A90B00. | 21 | 15 | 27 | 63 | Yes | Yes | Yes |
| | | | | | K99K99.R99R99. | F99F99.C99C99. | L90B00.L90B00. | | | | | | | |
| | | | | | A99A99.W99W99. | H99H99. | G90B00.N90B00. | | | | | | | |
| | | | | | S99S99. | | C90B00.I90B00. | | | | | | | |
| | | | | | | | C90B00. | | | | | | | |
| 15 | 9 | ATMSSEW | HAGYR | GQVDFTVCW | A99A99.T99T99. | H99H99.A99A99. | G99G99.Q99Q99. | 21 | 15 | 27 | 63 | Yes | Yes | Yes |
| | | | | | M99M99.S99S99. | G99G99.Y99Y99. | V99V99.D99D99. | | | | | | | |
| | | | | | S99S99.E99E99. | R99R99. | F99F99.T99T99. | | | | | | | |
| | | | | | W99W99. | | V99V99.C99C99. | | | | | | | |
| | | | | | | | W99W99. | | | | | | | |
| 172 | 9 | TIVQYYP | SAASN | SNGGYH | T99T99.I99I99. | S99S99.A99A99. | S99S99.N99N99. | 21 | 15 | 18 | 54 | Yes | Yes | Yes |
| | | | | | V99V99.Q99Q99. | A99A99.S99S99. | G99G99.G99G99. | | | | | | | |
| | | | | | Y99Y99.Y99Y99. | N99N99. | Y99Y99.H99H99. | | | | | | | |
| | | | | | P99P99. | | | | | | | | | |
| 176 | 9 | GLVAWAF | RMSIG | ITCNTISSGLWTF | G99G99.L99L99. | R99R99.M99M99. | I99I99.T99T99. | 21 | 15 | 39 | 75 | Yes | Yes | Yes |
| | | | | | V99V99.A99A99. | S99S99.I99I99. | C99C99.N99N99. | | | | | | | |
| | | | | | W99W99.A99A99. | G99G99. | T99T99.I99I99. | | | | | | | |
| | | | | | F99F99. | | S99S99.S99S99. | | | | | | | |
| | | | | | | | G99G99.L99L99. | | | | | | | |
| | | | | | | | W99W99.T99T99. | | | | | | | |
| | | | | | | | F99F99. | | | | | | | |
| 2 | 9 | PLLRVVY | VGSFV | SQFHHGRCSL | P99P99.L99L99. | V99V99.G99G99. | S99S99.Q99Q99. | 21 | 15 | 30 | 66 | Yes | Yes | Yes |
| | | | | | L99L99.R99R99. | S99S99.F99F99. | F99F99.H99H99. | | | | | | | |
| | | | | | V99V99.V99V99. | V99V99. | H99H99.G99G99. | | | | | | | |
| | | | | | Y99Y99. | | R99R99.C99C99. | | | | | | | |
| | | | | | | | S99S99.L99L99. | | | | | | | |
| 223 | 9 | AKNGMIG | LFRTV | LSWPDTSPLI | A99A99.K88B00. | L99L99.F99F99. | L99L99.S99S99. | 21 | 15 | 30 | 66 | Yes | Yes | Yes |
| | | | | | N99N99.G99G99. | R99R99.T99T99. | W99W99.P99P99. | | | | | | | |
| | | | | | M99M99.I99I99. | V99V99. | D99D99.T99T99. | | | | | | | |
| | | | | | G99G99. | | S99S99.P99P99. | | | | | | | |
| | | | | | | | L99L99.I99I99. | | | | | | | |
| 226 | 9 | CTFNVIW | LHNG | LIFDHNSGML | C99C99.T99T99. | L99L99.H99H99. | L99L99.I99I99. | 21 | 15 | 30 | 66 | Yes | Yes | Yes |
| | | | | | F99F99.N99N99. | N99N99.G99G99. | F99F99.D99D99. | | | | | | | |
| | | | | | V99V99.I99I99. | I99I99. | H99H99.N99N99. | | | | | | | |
| | | | | | W99W99. | | S99S99.G99G99. | | | | | | | |
| | | | | | | | M99M99.L99L99. | | | | | | | |
| 234 | 9 | FRS | MSDLQ | CIFIFHSLR | F99F99.R99R99. | M99M99.S99S99. | C99C99.I99I99. | 9 | 15 | 27 | 51 | Yes | Yes | Yes |
| | | | | | S99S99. | D99D99.L99L99. | F99F99.I99I99. | | | | | | | |
| | | | | | | Q99Q99. | F99F99.H99H99. | | | | | | | |
| | | | | | | | S99S99.L99L99. | | | | | | | |
| | | | | | | | R88B00. | | | | | | | |
| 241 | 9 | TFLPFIQ | GFTET | LFPQLSPRPA | T99T99.F99F99. | G99G99.F99F99. | L99L99.F99F99. | 21 | 15 | 30 | 66 | Yes | Yes | Yes |
| | | | | | L99L99.P99P99. | T99T99.E99E99. | P99P99.Q99Q99. | | | | | | | |
| | | | | | F99F99.I99I99. | T99T99. | L99L99.S99S99. | | | | | | | |
| | | | | | Q99Q99. | | P99P99.R88B00. | | | | | | | |
| | | | | | | | P99P99.A99A99. | | | | | | | |
| 243 | 9 | IMGLIRL | SNWLF | TTRFTH | I88B00.M99M99. | S99S99.N99N99. | T99T99.T99T99. | 21 | 15 | 18 | 54 | Yes | Yes | Yes |
| | | | | | G99G99.L99L99. | W99W99.L99L99. | R99R99.F99F99. | | | | | | | |
| | | | | | I99I99.R99R99. | F99F99. | T99T99.H99H99. | | | | | | | |
| | | | | | L99L99. | | | | | | | | | |
| 244 | 9 | VLGIVES | ARLEN | ITRFSG | V99V99.L99L99. | A99A99.R99R99. | I99I99.T99T99. | 21 | 15 | 18 | 54 | Yes | Yes | Yes |
| | | | | | G88B00.I88B00. | L99L99.E99E99. | R99R99.F99F99. | | | | | | | |
| | | | | | V88B00.E88B00. | N99N99. | S99S99.G99G99. | | | | | | | |
| | | | | | S88B00. | | | | | | | | | |
| 247 | 9 | SRTHPLR | TWGHV | SLPGTNGWAD | S99S99.R99R99. | T99T99.W99W99. | S99S99.L99L99. | 21 | 15 | 30 | 66 | Yes | Yes | Yes |
| | | | | | T88B00.H99H99. | G99G99.H99H99. | P99P99.G99G99. | | | | | | | |
| | | | | | P99P99.L99L99. | V99V99. | T99T99.N99N99. | | | | | | | |
| | | | | | R99R99. | | G99G99.W99W99. | | | | | | | |
| | | | | | | | A99A99.D99D99. | | | | | | | |
| 303 | 9 | DTNACVY | CHGVT | AFLMGARTI | D99D99.T99T99. | C99C99.H99H99. | A99A99.F99F99. | 21 | 15 | 27 | 63 | Yes | Yes | Yes |
| | | | | | N99N99.A99A99. | G99G99.V99V99. | L99L99.M99M99. | | | | | | | |
| | | | | | C99C99.V99V99. | T99T99. | G99G99.A99A99. | | | | | | | |
| | | | | | Y99Y99. | | R99R99.T99T99. | | | | | | | |
| | | | | | | | I99I99. | | | | | | | |
| 33 | 9 | ASRTVY | VSGLA | VIMQNIYVLK | A99A99.S99S99. | V99V99.S99S99. | V99V99.I99I99. | 18 | 15 | 30 | 63 | Yes | Yes | Yes |
| | | | | | R99R99.T99T99. | G99G99.L99L99. | M99M99.Q99Q99. | | | | | | | |
| | | | | | V99V99.Y99Y99. | A99A99. | N99N99.I99I99. | | | | | | | |
| | | | | | | | Y99Y99.V99V99. | | | | | | | |
| | | | | | | | L99L99.K99K99. | | | | | | | |
| 337 | 9 | LAGGVVY | AFGNG | AILSGRF | L99L99.A99A99. | A99A99.F99F99. | A99A99.I88B00. | 21 | 15 | 27 | 63 | Yes | Yes | Yes |
| | | | | | G99G99.G99G99. | G99G99.N99N99. | L99L99.S99S99. | | | | | | | |
| | | | | | V99V99.V99V99. | G99G99. | G88B00.R99R99. | | | | | | | |
| | | | | | Y99Y99. | | F99F99.V99V99. | | | | | | | |
| | | | | | | | C99C99. | | | | | | | |
| 34 | 9 | RLSGLVW | CDASL | KCYYSDAIV | R99R99.L99L99. | C99C99.D99D99. | B00 | 21 | 15 | 0 | 36 | Yes | Yes | Yes |
| | | | | | S99S99.G99G99. | A99A99.S99S99. | | | | | | | | |
| | | | | | L99L99.V99V99. | L99L99. | | | | | | | | |
| | | | | | W99W99. | | | | | | | | | |
| 347 | 9 | PLVRACY | LSESY | AIFDPTQEGGGAV | P99P99.L99L99. | L99L99.S99S99. | A99A99.I99I99. | 21 | 15 | 39 | 75 | Yes | Yes | Yes |
| | | | | | V99V99.R99R99. | E99E99.S99S99. | F99F99.D99D99. | | | | | | | |
| | | | | | A99A99.C99C99. | Y99Y99. | P99P99.T88B00. | | | | | | | |
| | | | | | Y88B00. | | Q99Q99.E99E99. | | | | | | | |
| | | | | | | | G99G99.G99G99. | | | | | | | |
| | | | | | | | G99G99.A99A99. | | | | | | | |
| | | | | | | | V99V99. | | | | | | | |
| 349 | 9 | SVTARVD | HVPFA | FSANCRDISR | S99S99.V99V99. | H99H99.V99V99. | F99F99.S99S99. | 21 | 15 | 30 | 66 | Yes | Yes | Yes |
| | | | | | T99T99.A99A99. | P99P99.F99F99. | A99A99.N99N99. | | | | | | | |
| | | | | | R99R99.V99V99. | A99A99. | C99C99.R99R99. | | | | | | | |
| | | | | | D88B00. | | D99D99.I99I99. | | | | | | | |
| | | | | | | | S99S99.R99R99. | | | | | | | |
| 385 | 9 | CVLYVF | ANNAE | ALYSLFSTR | C99C99.V99V99. | A99A99.N99N99. | A99A99.L99L99. | 18 | 15 | 27 | 60 | Yes | Yes | Yes |
| | | | | | L99L99.Y99Y99. | N99N99.A99A99. | Y99Y99.S99S99. | | | | | | | |
| | | | | | V99V99.F99F99. | E99E99. | L99L99.F99F99. | | | | | | | |
| | | | | | | | S99S99.T99T99. | | | | | | | |
| | | | | | | | R99R99. | | | | | | | |
| 392 | 9 | ISVSFWF | PCLNN | WTPTRLVVV | I88B00.S99S99. | P99P99.C99C99. | W99W99.T99T99. | 21 | 15 | 27 | 63 | Yes | Yes | Yes |
| | | | | | V99V99.S99S99. | L99L99.N99N99. | P99P99.T99T99. | | | | | | | |
| | | | | | F99F99.W88B00. | N99N99. | R99R99.L99L99. | | | | | | | |
| | | | | | F99F99. | | V99V99.V99V99. | | | | | | | |
| | | | | | | | V99V99. | | | | | | | |
| 432 | 9 | RGVGTVY | PCDDG | GIYYDCHCIT | R99R99.G99G99. | P99P99.C99C99. | G99G99.I99I99. | 21 | 15 | 30 | 66 | Yes | Yes | Yes |
| | | | | | V99V99.G99G99. | D99D99.D99D99. | Y99Y99.Y99Y99. | | | | | | | |
| | | | | | T99T99.V99V99. | G99G99. | D99D99.C99C99. | | | | | | | |
| | | | | | Y99Y99. | | H99H99.C99C99. | | | | | | | |
| | | | | | | | I99I99.T99T99. | | | | | | | |
| 433 | 9 | CLICYST | DVHAL | TTGYDTGALP | C99C99.L99L99. | D99D99.V99V99. | T99T99.T88B00. | 21 | 15 | 30 | 66 | Yes | Yes | Yes |
| | | | | | I99I99.C99C99. | H99H99.A99A99. | G99G99.Y99Y99. | | | | | | | |
| | | | | | Y99Y99.S99S99. | L99L99. | D99D99.T99T99. | | | | | | | |
| | | | | | T99T99. | | G88B00.A99A99. | | | | | | | |
| | | | | | | | L99L99.P99P99. | | | | | | | |
| 448 | 9 | TMLHIFS | SLYPH | TVMCYVMGFSGTD | T99T99.M99M99. | S99S99.L99L99. | T99T99.V99V99. | 21 | 15 | 39 | 75 | Yes | Yes | Yes |
| | | | | | L99L99.H99H99. | Y99Y99.P99P99. | M99M99.C99C99. | | | | | | | |
| | | | | | I99I99.F99F99. | H99H99. | Y99Y99.V99V99. | | | | | | | |
| | | | | | S99S99. | | M99M99.G99G99. | | | | | | | |
| | | | | | | | F99F99.S99S99. | | | | | | | |
| | | | | | | | G99G99.T99T99. | | | | | | | |
| | | | | | | | D99D99. | | | | | | | |
| 466 | 9 | CVLMATS | GIDMT | HDFHPHYRGG | C99C99.V99V99. | G99G99.I99I99. | H77B00.D66B00. | 21 | 15 | 20 | 56 | Yes | Yes | Yes |
| | | | | | L99L99.M99M99. | D99D99.M99M99. | F77B00.H77B00. | | | | | | | |
| | | | | | A99A99.T99T99. | T99T99. | P77B00.H77B00. | | | | | | | |
| | | | | | S88B00. | | Y77B00.R77B00. | | | | | | | |
| | | | | | | | G77B00.G77B00. | | | | | | | |
| 472 | 9 | QALSCFN | SSSTS | FPTPNSAGVF | Q99Q99.A99A99. | S99S99.S99S99. | F99F99.P99P99. | 21 | 15 | 30 | 66 | Yes | Yes | Yes |
| | | | | | L99L99.S99S99. | S99S99.T99T99. | T99T99.P99P99. | | | | | | | |
| | | | | | C99C99.F99F99. | S99S99. | N99N99.S99S99. | | | | | | | |
| | | | | | N99N99. | | A99A99.G99G99. | | | | | | | |
| | | | | | | | V99V99.F99F99. | | | | | | | |
| 483 | 9 | AHARTVW | RSGGG | MCLLCNGVI | A99A99.H99H99. | R99R99.S99S99. | M99M99.C99C99. | 21 | 15 | 27 | 63 | Yes | Yes | Yes |
| | | | | | A99A99.R99R99. | G99G99.G99G99. | L99L99.L99L99. | | | | | | | |
| | | | | | T99T99.V99V99. | G99G99. | C99C99.N99N99. | | | | | | | |
| | | | | | W99W99. | | G99G99.V99V99. | | | | | | | |
| | | | | | | | I99I99. | | | | | | | |
| 49 | 9 | CLSNYLN | MSFSL | VYCIDHSVSTDVL | C99C99.L99L99. | M99M99.S99S99. | B00 | 21 | 15 | 0 | 36 | Yes | Yes | Yes |
| | | | | | S99S99.N99N99. | F99F99.S99S99. | | | | | | | | |
| | | | | | Y99Y99.L99L99. | L99L99. | | | | | | | | |
| | | | | | N99N99. | | | | | | | | | |
| 496 | 9 | AGSRCVY | PRVET | YLLVDGIVV | A99A99.G99G99. | P99P99.R99R99. | Y99Y99.L99L99. | 21 | 15 | 27 | 63 | Yes | Yes | Yes |
| | | | | | S99S99.R99R99. | V99V99.E99E99. | L99L99.V99V99. | | | | | | | |
| | | | | | C99C99.V99V99. | T88B00. | D99D99.G99G99. | | | | | | | |
| | | | | | Y99Y99. | | I99I99.V99V99. | | | | | | | |
| | | | | | | | V88B00. | | | | | | | |
| 498 | 9 | DVVNYYT | AHVGF | HIGTVS | D99D99.V99V99. | A99A99.H99H99. | H99H99.I99I99. | 21 | 15 | 18 | 54 | Yes | Yes | Yes |
| | | | | | V99V99.N99N99. | V99V99.G99G99. | G99G99.T99T99. | | | | | | | |
| | | | | | Y99Y99.Y88B00. | F99F99. | V99V99.S99S99. | | | | | | | |
| | | | | | T99T99. | | | | | | | | | |
| 508 | 9 | ICTLTCW | LVQVM | RHLIRDLNGVLVR | I99I99.C99C99. | L99L99.V99V99. | R99R99.H99H99. | 20 | 15 | 39 | 74 | Yes | Yes | Yes |
| | | | | | T77B00.L99L99. | Q99Q99.V99V99. | L99L99.I99I99. | | | | | | | |
| | | | | | T99T99.C99C99. | M99M99. | R99R99.D99D99. | | | | | | | |
| | | | | | W99W99. | | L99L99.N99N99. | | | | | | | |
| | | | | | | | G99G99.V99V99. | | | | | | | |
| | | | | | | | L99L99.V99V99. | | | | | | | |
| | | | | | | | R99R99. | | | | | | | |
| 51 | 9 | YSGRMVW | MATDN | CSEWW | Y99Y99.S99S99. | M99M99.A99A99. | C99C99.S99S99. | 21 | 15 | 15 | 51 | Yes | Yes | Yes |
| | | | | | G99G99.R99R99. | T99T99.D99D99. | E99E99.W99W99. | | | | | | | |
| | | | | | M99M99.V99V99. | N99N99. | W99W99. | | | | | | | |
| | | | | | W99W99. | | | | | | | | | |
| 53 | 9 | TYLTMLG | HQSCY | SVFLINLSVV | T99T99.Y99Y99. | H99H99.Q99Q99. | S99S99.V99V99. | 21 | 15 | 30 | 66 | Yes | Yes | Yes |
| | | | | | L99L99.T99T99. | S99S99.C99C99. | F99F99.L99L99. | | | | | | | |
| | | | | | M99M99.L99L99. | Y99Y99. | I99I99.N99N99. | | | | | | | |
| | | | | | G99G99. | | L99L99.S99S99. | | | | | | | |
| | | | | | | | V99V99.V99V99. | | | | | | | |
| 522 | 9 | VLYGEVY | QGCYD | AVFHPDLGVH | V99V99.L99L99. | Q99Q99.G99G99. | A99A99.V99V99. | 21 | 15 | 30 | 66 | Yes | Yes | Yes |
| | | | | | Y99Y99.G99G99. | C99C99.Y99Y99. | F99F99.H99H99. | | | | | | | |
| | | | | | E99E99.V99V99. | D99D99. | P99P99.D99D99. | | | | | | | |
| | | | | | Y99Y99. | | L99L99.G99G99. | | | | | | | |
| | | | | | | | V99V99.H99H99. | | | | | | | |
| 55 | 9 | SGLRVMG | IGLAL | CILSYRRAEEAE | S88B00.G99G99. | I99I99.G99G99. | C99C99.I99I99. | 21 | 15 | 36 | 72 | Yes | Yes | Yes |
| | | | | | L99L99.R99R99. | L99L99.A99A99. | L99L99.S99S99. | | | | | | | |
| | | | | | V99V99.M99M99. | L99L99. | Y99Y99.R99R99. | | | | | | | |
| | | | | | G99G99. | | R99R99.A99A99. | | | | | | | |
| | | | | | | | E99E99.E99E99. | | | | | | | |
| | | | | | | | A99A99.E99E99. | | | | | | | |
| 564 | 9 | HARGGAC | VWADQ | FHSSQS | H99H99.A99A99. | V99V99.W99W99. | F99F99.H99H99. | 21 | 15 | 18 | 54 | Yes | Yes | Yes |
| | | | | | R99R99.G99G99. | A99A99.D99D99. | S99S99.S99S99. | | | | | | | |
| | | | | | G99G99.A99A99. | Q99Q99. | Q99Q99.S99S99. | | | | | | | |
| | | | | | C99C99. | | | | | | | | | |
| 569 | 9 | IQSMFHY | PRYS | DNTPIMIG | I99I99.Q99Q99. | P99P99.R99R99. | B00 | 21 | 12 | 0 | 33 | Yes | Yes | Yes |
| | | | | | S99S99.M99M99. | Y99Y99.S99S99. | | | | | | | | |
| | | | | | F99F99.H99H99. | | | | | | | | | |
| | | | | | Y99Y99. | | | | | | | | | |
| 597 | 9 | SGYRVVY | TDAKA | VLFPSC | S99S99.G99G99. | T99T99.D99D99. | B00 | 21 | 15 | 0 | 36 | Yes | Yes | Yes |
| | | | | | Y99Y99.R99R99. | A99A99.K99K99. | | | | | | | | |
| | | | | | V99V99.V99V99. | A99A99. | | | | | | | | |
| | | | | | Y99Y99. | | | | | | | | | |
| 601 | 9 | LSGGVAI | VQEYC | TNFLSRNAS | L99L99.S99S99. | V99V99.Q99Q99. | T99T99.N99N99. | 21 | 15 | 27 | 63 | Yes | Yes | Yes |
| | | | | | G99G99.G99G99. | E99E99.Y99Y99. | F99F99.L99L99. | | | | | | | |
| | | | | | V99V99.A99A99. | C99C99. | S99S99.R99R99. | | | | | | | |
| | | | | | I88B00. | | N99N99.A99A99. | | | | | | | |
| | | | | | | | S99S99. | | | | | | | |
| 648 | 9 | VTFSLFR | VSNCA | TGSPIPFIYFAHG | V99V99.T99T99. | V99V99.S99S99. | T99T99.G99G99. | 21 | 15 | 39 | 75 | Yes | Yes | Yes |
| | | | | | F99F99.S99S99. | N99N99.C99C99. | S99S99.P99P99. | | | | | | | |
| | | | | | L99L99.F99F99. | A88B00. | I99I99.P99P99. | | | | | | | |
| | | | | | R99R99. | | F99F99.I99I99. | | | | | | | |
| | | | | | | | Y99Y99.F99F99. | | | | | | | |
| | | | | | | | A99A99.H99H99. | | | | | | | |
| | | | | | | | G99G99. | | | | | | | |
| 672 | 9 | AYTNLCC | FYLGV | ERYPYS | A99A99.Y99Y99. | F99F99.Y99Y99. | E99E99.R99R99. | 21 | 15 | 18 | 54 | Yes | Yes | Yes |
| | | | | | T99T99.N99N99. | L99L99.G99G99. | Y99Y99.P99P99. | | | | | | | |
| | | | | | L99L99.C99C99. | V99V99. | Y99Y99.S99S99. | | | | | | | |
| | | | | | C99C99. | | | | | | | | | |
| 676 | 9 | ILVCYTV | GNVRC | MHIYGE | I99I99.L99L99. | G99G99.N99N99. | B00 | 21 | 15 | 0 | 36 | Yes | Yes | Yes |
| | | | | | V99V99.C99C99. | V99V99.R99R99. | | | | | | | | |
| | | | | | Y99Y99.T99T99. | C99C99. | | | | | | | | |
| | | | | | V99V99. | | | | | | | | | |
| 679 | 9 | FASNRRF | GVHRI | RGDPLHPVWY | F99F99.A99A99. | G99G99.V99V99. | R99R99.G99G99. | 21 | 15 | 30 | 66 | Yes | Yes | Yes |
| | | | | | S99S99.N99N99. | H99H99.R99R99. | D99D99.P99P99. | | | | | | | |
| | | | | | R99R99.R99R99. | I99I99. | L99L99.H99H99. | | | | | | | |
| | | | | | F99F99. | | P99P99.V99V99. | | | | | | | |
| | | | | | | | W99W99.Y99Y99. | | | | | | | |
| 681 | 9 | PVRCFVW | CVSAF | LHHTNDDNYR | P99P99.V99V99. | C99C99.V99V99. | L99L99.H99H99. | 21 | 15 | 30 | 66 | Yes | Yes | Yes |
| | | | | | R99R99.C99C99. | S99S99.A99A99. | H99H99.T99T99. | | | | | | | |
| | | | | | F99F99.V99V99. | F99F99. | N99N99.D99D99. | | | | | | | |
| | | | | | W99W99. | | D99D99.N99N99. | | | | | | | |
| | | | | | | | Y99Y99.R99R99. | | | | | | | |
| 706 | 9 | MVKRWVY | RSVAS | GLLDDHACFGHGR | M99M99.V99V99. | R99R99.S99S99. | G99G99.L99L99. | 21 | 15 | 39 | 75 | Yes | Yes | Yes |
| | | | | | K99K99.R99R99. | V99V99.A99A99. | L99L99.D99D99. | | | | | | | |
| | | | | | W99W99.V99V99. | S99S99. | D99D99.H99H99. | | | | | | | |
| | | | | | Y99Y99. | | A99A99.C99C99. | | | | | | | |
| | | | | | | | F99F99.G99G99. | | | | | | | |
| | | | | | | | H99H99.G99G99. | | | | | | | |
| | | | | | | | R99R99. | | | | | | | |
| 70 | 9 | GVSSIVV | RHAQH | QALTANFFVS | G99G99.V99V99. | R99R99.H99H99. | Q99Q99.A99A99. | 21 | 15 | 30 | 66 | Yes | Yes | Yes |
| | | | | | S99S99.S99S99. | A99A99.Q99Q99. | L99L99.T99T99. | | | | | | | |
| | | | | | I99I99.V99V99. | H99H99. | A99A99.N99N99. | | | | | | | |
| | | | | | V99V99. | | F99F99.F99F99. | | | | | | | |
| | | | | | | | V99V99.S99S99. | | | | | | | |
| 715 | 9 | YSFAADQ | VHCDA | LAPRFGFVAW | Y99Y99.S99S99. | V99V99.H99H99. | L99L99.A88B00. | 21 | 15 | 30 | 66 | Yes | Yes | Yes |
| | | | | | F99F99.A99A99. | C99C99.D99D99. | P88B00.R88B00. | | | | | | | |
| | | | | | A99A99.D99D99. | A99A99. | F88B00.G88B00. | | | | | | | |
| | | | | | Q99Q99. | | F88B00.V88B00. | | | | | | | |
| | | | | | | | A88B00.W88B00. | | | | | | | |
| 718 | 9 | GSGYVGY | VDGVP | VLIHRINGSFAML | G99G99.S99S99. | V99V99.D99D99. | V99V99.L88B00. | 21 | 15 | 39 | 75 | Yes | Yes | Yes |
| | | | | | G99G99.Y99Y99. | G99G99.V99V99. | I99I99.H99H99. | | | | | | | |
| | | | | | V99V99.G99G99. | P99P99. | R99R99.I99I99. | | | | | | | |
| | | | | | Y99Y99. | | N99N99.G99G99. | | | | | | | |
| | | | | | | | S99S99.F99F99. | | | | | | | |
| | | | | | | | A99A99.M99M99. | | | | | | | |
| | | | | | | | L99L99. | | | | | | | |
| 722 | 9 | NIWYHGQ | LTHMG | MRFFEN | N99N99.I99I99. | L99L99.T99T99. | M99M99.R99R99. | 21 | 15 | 18 | 54 | Yes | Yes | Yes |
| | | | | | W99W99.Y99Y99. | H99H99.M99M99. | F99F99.F99F99. | | | | | | | |
| | | | | | H99H99.G99G99. | G99G99. | E99E99.N99N99. | | | | | | | |
| | | | | | Q99Q99. | | | | | | | | | |
| 726 | 9 | CVRSSVY | VGMRC | AYYRSSIMVE | C99C99.V99V99. | V99V99.G99G99. | A99A99.Y99Y99. | 21 | 15 | 30 | 66 | Yes | Yes | Yes |
| | | | | | R99R99.S99S99. | M99M99.R99R99. | Y99Y99.R99R99. | | | | | | | |
| | | | | | S99S99.V99V99. | C99C99. | S99S99.S99S99. | | | | | | | |
| | | | | | Y99Y99. | | I99I99.M99M99. | | | | | | | |
| | | | | | | | V99V99.E99E99. | | | | | | | |
| 728 | 9 | GRVIA | CHPSQ | HYIVSNYLMVYVC | G99G99.R99R99. | C99C99.H99H99. | H99H99.Y99Y99. | 15 | 15 | 39 | 69 | Yes | Yes | Yes |
| | | | | | V99V99.I99I99. | P99P99.S99S99. | I99I99.V99V99. | | | | | | | |
| | | | | | A99A99. | Q99Q99. | S99S99.N99N99. | | | | | | | |
| | | | | | | | Y99Y99.L99L99. | | | | | | | |
| | | | | | | | M99M99.V99V99. | | | | | | | |
| | | | | | | | Y99Y99.V99V99. | | | | | | | |
| | | | | | | | C99C99. | | | | | | | |
| 733 | 9 | SLRGLLV | SSGVT | YMFTPRSDVS | S99S99.L99L99. | S99S99.S99S99. | Y99Y99.M99M99. | 21 | 15 | 30 | 66 | Yes | Yes | Yes |
| | | | | | R99R99.G99G99. | G99G99.V99V99. | F99F99.T99T99. | | | | | | | |
| | | | | | L99L99.L99L99. | T99T99. | P99P99.R99R99. | | | | | | | |
| | | | | | V99V99. | | S99S99.D99D99. | | | | | | | |
| | | | | | | | V99V99.S99S99. | | | | | | | |
| 738 | 9 | TSSWEW | NRGFR | LASGTNGVFF | T99T99.S99S99. | N99N99.R99R99. | L99L99.A99A99. | 18 | 15 | 30 | 63 | Yes | Yes | Yes |
| | | | | | S99S99.W99W99. | G99G99.F99F99. | S99S99.G99G99. | | | | | | | |
| | | | | | E99E99.W99W99. | R99R99. | T99T99.N99N99. | | | | | | | |
| | | | | | | | G99G99.V99V99. | | | | | | | |
| | | | | | | | F99F99.F99F99. | | | | | | | |
| 749 | 9 | AFVWSAV | SQLTL | FELPDHFSFS | A99A99.F99F99. | S99S99.Q99Q99. | F99F99.E99E99. | 21 | 15 | 30 | 66 | Yes | Yes | Yes |
| | | | | | V99V99.W99W99. | L99L99.T99T99. | L99L99.P99P99. | | | | | | | |
| | | | | | S99S99.A99A99. | L99L99. | D99D99.H99H99. | | | | | | | |
| | | | | | V99V99. | | F99F99.S99S99. | | | | | | | |
| | | | | | | | F99F99.S99S99. | | | | | | | |
| 804 | 9 | LQRVVH | VCAAM | VKHVSHLQTV | L99L99.Q99Q99. | V99V99.C99C99. | V88B00.K88B00. | 18 | 15 | 29 | 62 | Yes | Yes | Yes |
| | | | | | R99R99.V99V99. | A99A99.A99A99. | H88B00.V88B00. | | | | | | | |
| | | | | | V99V99.H99H99. | M99M99. | S77B00.H88B00. | | | | | | | |
| | | | | | | | L88B00.Q88B00. | | | | | | | |
| | | | | | | | T88B00.V88B00. | | | | | | | |
| 805 | 9 | CHLVY | KGDGM | AYMTLVDVT | C99C99.H99H99. | K99K99.G99G99. | A99A99.Y99Y99. | 15 | 15 | 27 | 57 | Yes | Yes | Yes |
| | | | | | L99L99.V99V99. | D99D99.G99G99. | M99M99.T99T99. | | | | | | | |
| | | | | | Y99Y99. | M99M99. | L99L99.V99V99. | | | | | | | |
| | | | | | | | D99D99.V99V99. | | | | | | | |
| | | | | | | | T99T99. | | | | | | | |
| 818 | 9 | SWHIVGA | CYTSS | AINPICIAYMALL | S88B00.W99W99. | C99C99.Y99Y99. | A99A99.I99I99. | 21 | 15 | 39 | 75 | Yes | Yes | Yes |
| | | | | | H99H99.I99I99. | T99T99.S99S99. | N99N99.P99P99. | | | | | | | |
| | | | | | V99V99.G99G99. | S99S99. | I99I99.C99C99. | | | | | | | |
| | | | | | A99A99. | | I99I99.A99A99. | | | | | | | |
| | | | | | | | Y99Y99.M99M99. | | | | | | | |
| | | | | | | | A99A99.L99L99. | | | | | | | |
| | | | | | | | L99L99. | | | | | | | |
| 830 | 9 | VTINSN | RHVDV | FSIPTFGYVAPVR | V99V99.T99T99. | R99R99.H99H99. | F99F99.S99S99. | 18 | 15 | 39 | 72 | Yes | Yes | Yes |
| | | | | | I99I99.N99N99. | V99V99.D99D99. | I99I99.P99P99. | | | | | | | |
| | | | | | S88B00.N99N99. | V99V99. | T99T99.F99F99. | | | | | | | |
| | | | | | | | G99G99.Y99Y99. | | | | | | | |
| | | | | | | | V99V99.A99A99. | | | | | | | |
| | | | | | | | P99P99.V99V99. | | | | | | | |
| | | | | | | | R99R99. | | | | | | | |
| 87 | 9 | HDGLVT | DYYER | FISVCI | H99H99.D99D99. | D99D99.Y99Y99. | F99F99.I99I99. | 21 | 15 | 18 | 54 | Yes | Yes | Yes |
| | | | | | G99G99.L99L99. | Y99Y99.E99E99. | S99S99.V99V99. | | | | | | | |
| | | | | | R99R99.V99V99. | R99R99. | C99C99.I99I99. | | | | | | | |
| | | | | | T99T99. | | | | | | | | | |
| 88 | 9 | VFAKVKG | AF | DTNWDYVYPS | V99V99.F99F99. | A99A99.F99F99. | D88B00.T99T99. | 21 | 6 | 30 | 57 | Yes | Yes | Yes |
| | | | | | A99A99.K99K99. | | N99N99.W99W99. | | | | | | | |
| | | | | | V99V99.K99K99. | | D99D99.Y99Y99. | | | | | | | |
| | | | | | G99G99. | | V99V99.Y99Y99. | | | | | | | |
| | | | | | | | P99P99.S99S99. | | | | | | | |
| 882 | 9 | TTCQCCF | VCEFL | AYVRGASVT | T99T99.T99T99. | V99V99.C99C99. | A99A99.Y99Y99. | 21 | 15 | 27 | 63 | Yes | Yes | Yes |
| | | | | | C99C99.Q99Q99. | E99E99.F99F99. | V99V99.R99R99. | | | | | | | |
| | | | | | C99C99.C99C99. | L99L99. | G99G99.A99A99. | | | | | | | |
| | | | | | F99F99. | | S99S99.V99V99. | | | | | | | |
| | | | | | | | T99T99. | | | | | | | |
| 154 | 8 | VRCLDAF | IQLGD | QAFGCWGHV | V99V99.R99R99. | I99I99.Q99Q99. | Q99Q99.A99A99. | 21 | 15 | 27 | 63 | Yes | Yes | Yes |
| | | | | | C99C99.L99L99. | L99L99.G99G99. | F99F99.G99G99. | | | | | | | |
| | | | | | D99D99.A99A99. | D99D99. | C99C99.W99W99. | | | | | | | |
| | | | | | F99F99. | | G99G99.H99H99. | | | | | | | |
| | | | | | | | V99V99. | | | | | | | |
| 159 | 8 | NLCSF | QFSSD | ANTSSMVKPV | N99N99.L99L99. | Q99Q99.F99F99. | A99A99.N99N99. | 15 | 15 | 30 | 60 | Yes | Yes | Yes |
| | | | | | C99C99.S99S99. | S99S99.S99S99. | T99T99.S99S99. | | | | | | | |
| | | | | | F99F99. | D99D99. | S99S99.M99M99. | | | | | | | |
| | | | | | | | V99V99.K99K99. | | | | | | | |
| | | | | | | | P99P99.V99V99. | | | | | | | |
| 225 | 8 | FLNSLVF | TDSYV | AVYLRRGSVA | F99F99.L99L99. | T99T99.D99D99. | A87B00.V99V99. | 21 | 15 | 30 | 66 | Yes | Yes | Yes |
| | | | | | N99N99.S99S99. | S99S99.Y99Y99. | Y99Y99.L99L99. | | | | | | | |
| | | | | | L87B00.V99V99. | V99V99. | R99R99.R99R99. | | | | | | | |
| | | | | | F99F99. | | G99G99.S99S99. | | | | | | | |
| | | | | | | | V99V99.A99A99. | | | | | | | |
| 306 | 8 | SFGLVRF | EAAAG | IYSLDDVGCM | S99S99.F99F99. | E99E99.A99A99. | I99I99.Y99Y99. | 21 | 15 | 30 | 66 | Yes | Yes | Yes |
| | | | | | G99G99.L99L99. | A99A99.A99A99. | S99S99.L99L99. | | | | | | | |
| | | | | | V99V99.R99R99. | G99G99. | D99D99.D99D99. | | | | | | | |
| | | | | | F99F99. | | V99V99.G99G99. | | | | | | | |
| | | | | | | | C99C99.M99M99. | | | | | | | |
| 311 | 8 | GRCYMSS | MAHSL | VDSYY | G99G99.R99R99. | M99M99.A99A99. | V99V99.D99D99. | 21 | 15 | 15 | 51 | Yes | Yes | Yes |
| | | | | | C99C99.Y99Y99. | H99H99.S99S99. | S99S99.Y99Y99. | | | | | | | |
| | | | | | M87B00.S99S99. | L99L99. | Y99Y99. | | | | | | | |
| | | | | | S99S99. | | | | | | | | | |
| 312 | 8 | SVAALRF | YATSR | PLHFHY | S99S99.V99V99. | Y99Y99.A99A99. | P99P99.L99L99. | 21 | 15 | 18 | 54 | Yes | Yes | Yes |
| | | | | | A99A99.A99A99. | T99T99.S99S99. | H99H99.F99F99. | | | | | | | |
| | | | | | L99L99.R99R99. | R99R99. | H99H99.Y99Y99. | | | | | | | |
| | | | | | F99F99. | | | | | | | | | |
| 324 | 8 | GSTGQVY | RDGFI | YHFVAGNVN | G87B00.S87B00. | R99R99.D99D99. | Y99Y99.H99H99. | 20 | 15 | 27 | 62 | Yes | Yes | Yes |
| | | | | | T75B00.G87B00. | G99G99.F99F99. | F99F99.V99V99. | | | | | | | |
| | | | | | Q87B00.V87B00. | I99I99. | A99A99.G99G99. | | | | | | | |
| | | | | | Y87B00. | | N99N99.V99V99. | | | | | | | |
| | | | | | | | N99N99. | | | | | | | |
| 328 | 8 | TSCPFQC | ITWCV | EDMEGY | T99T99.S99S99. | I99I99.T99T99. | E87B00.D87B00. | 21 | 15 | 18 | 54 | Yes | Yes | Yes |
| | | | | | C99C99.P99P99. | W99W99.C99C99. | M99M99.E87B00. | | | | | | | |
| | | | | | F99F99.Q99Q99. | V99V99. | G87B00.Y87B00. | | | | | | | |
| | | | | | C99C99. | | | | | | | | | |
| 339 | 8 | MNRKAR | TDGSL | LVEFL | M99M99.N99N99. | T99T99.D99D99. | L75B00.V75B00. | 18 | 15 | 10 | 43 | Yes | Yes | Yes |
| | | | | | R99R99.K99K99. | G99G99.S99S99. | E75B00.F75B00. | | | | | | | |
| | | | | | A99A99.R99R99. | L99L99. | L75B00. | | | | | | | |
| 341 | 8 | LVCCT | HWFKS | FTSCIT | L99L99.V99V99. | H99H99.W99W99. | F99F99.T99T99. | 21 | 15 | 18 | 54 | Yes | Yes | Yes |
| | | | | | C99C99.C99C99. | F99F99.K99K99. | S99S99.C99C99. | | | | | | | |
| | | | | | S99S99.C99C99. | S99S99. | I99I99.T99T99. | | | | | | | |
| | | | | | T99T99. | | | | | | | | | |
| 348 | 8 | EFVRVFA | LGSHD | TIMQSPSLVH | E99E99.F99F99. | L99L99.G99G99. | T99T99.I99I99. | 21 | 15 | 30 | 66 | Yes | Yes | Yes |
| | | | | | V99V99.R99R99. | S99S99.H99H99. | M99M99.Q99Q99. | | | | | | | |
| | | | | | V99V99.F99F99. | D99D99. | S99S99.P99P99. | | | | | | | |
| | | | | | A99A99. | | S99S99.L99L99. | | | | | | | |
| | | | | | | | V99V99.H99H99. | | | | | | | |
| 403 | 8 | CAYNWFC | VTTSH | VDDFG | C99C99.A99A99. | V99V99.T99T99. | V99V99.D99D99. | 21 | 15 | 18 | 54 | Yes | Yes | Yes |
| | | | | | Y99Y99.N99N99. | T99T99.S99S99. | D99D99.F99F99. | | | | | | | |
| | | | | | W99W99.F99F99. | H99H99. | G99G99.I99I99. | | | | | | | |
| | | | | | C99C99. | | | | | | | | | |
| 438 | 8 | ATCDAFR | TTIDS | SHFNDTTRM | A99A99.T99T99. | T99T99.T99T99. | B00 | 21 | 15 | 0 | 36 | Yes | Yes | Yes |
| | | | | | C99C99.D99D99. | I99I99.D99D99. | | | | | | | | |
| | | | | | A99A99.F99F99. | S99S99. | | | | | | | | |
| | | | | | R99R99. | | | | | | | | | |
| 486 | 8 | TFARCLV | LHFLN | TYTHDGYYLS | T99T99.F99F99. | L99L99.H99H99. | B00 | 21 | 15 | 0 | 36 | Yes | Yes | Yes |
| | | | | | A99A99.R99R99. | F99F99.L99L99. | | | | | | | | |
| | | | | | C99C99.L99L99. | N99N99. | | | | | | | | |
| | | | | | V99V99. | | | | | | | | | |
| 545 | 8 | NILYVFG | GSHKV | NAILSTNTHD | N87B00.I99I99. | G99G99.S99S99. | N99N99.A99A99. | 21 | 15 | 30 | 66 | Yes | Yes | Yes |
| | | | | | L99L99.Y99Y99. | H99H99.K99K99. | I99I99.L99L99. | | | | | | | |
| | | | | | V99V99.F99F99. | V99V99. | S99S99.T99T99. | | | | | | | |
| | | | | | G99G99. | | N99N99.T99T99. | | | | | | | |
| | | | | | | | H99H99.D99D99. | | | | | | | |
| 551 | 8 | HCGCVF | PARCH | SIYLNENVIL | H87B00.I87B00. | P99P99.A99A99. | S99S99.I99I99. | 21 | 15 | 30 | 66 | Yes | Yes | Yes |
| | | | | | C87B00.G87B00. | R99R99.C99C99. | Y99Y99.L99L99. | | | | | | | |
| | | | | | C87B00.V87B00. | H99H99. | N99N99.E99E99. | | | | | | | |
| | | | | | F87B00. | | N99N99.V99V99. | | | | | | | |
| | | | | | | | I87B00.L99L99. | | | | | | | |
| 560 | 8 | KSMIW | VTGGR | VTFHNECIVR | K99K99.S99S99. | V99V99.T99T99. | V99V99.T99T99. | 15 | 15 | 30 | 60 | Yes | Yes | Yes |
| | | | | | M99M99.I99I99. | G99G99.G99G99. | F99F99.H99H99. | | | | | | | |
| | | | | | W99W99. | R99R99. | N99N99.E99E99. | | | | | | | |
| | | | | | | | C99C99.I99I99. | | | | | | | |
| | | | | | | | V99V99.R99R99. | | | | | | | |
| 581 | 8 | GTPGR | SCVAS | RDTAFSLCQNGWT | G99G99.T99T99. | S99S99.C99C99. | R99R99.D99D99. | 15 | 15 | 39 | 69 | Yes | Yes | Yes |
| | | | | | P99P99.G99G99. | V99V99.A99A99. | T99T99.A99A99. | | | | | | | |
| | | | | | R99R99. | S99S99. | F99F99.S99S99. | | | | | | | |
| | | | | | | | L99L99.C99C99. | | | | | | | |
| | | | | | | | Q99Q99.N99N99. | | | | | | | |
| | | | | | | | G99G99.W99W99. | | | | | | | |
| | | | | | | | T99T99. | | | | | | | |
| 583 | 8 | AHGGLVY | RGGVL | AYYELG | A99A99.H99H99. | R99R99.G99G99. | A99A99.Y99Y99. | 21 | 15 | 18 | 54 | Yes | Yes | Yes |
| | | | | | G99G99.G99G99. | G99G99.V99V99. | Y99Y99.E99E99. | | | | | | | |
| | | | | | L99L99.V99V99. | L99L99. | L99L99.G99G99. | | | | | | | |
| | | | | | Y99Y99. | | | | | | | | | |
| 585 | 8 | GTRGCIC | HSCTD | LFLDSSGLVF | G99G99.T99T99. | H99H99.S99S99. | L87B00.F99F99. | 21 | 15 | 30 | 66 | Yes | Yes | Yes |
| | | | | | R99R99.G99G99. | C99C99.T99T99. | L99L99.D87B00. | | | | | | | |
| | | | | | C99C99.I99I99. | D99D99. | S99S99.S87B00. | | | | | | | |
| | | | | | C99C99. | | G99G99.L99L99. | | | | | | | |
| | | | | | | | V99V99.F99F99. | | | | | | | |
| 587 | 8 | GSWQWM | SVSCF | VVGRCTNMHW | G99G99.S99S99. | S99S99.V99V99. | V99V99.V99V99. | 21 | 15 | 30 | 66 | Yes | Yes | Yes |
| | | | | | L99L99.W99W99. | S99S99.C99C99. | G99G99.R99R99. | | | | | | | |
| | | | | | Q99Q99.W99W99. | F99F99. | C99C99.T99T99. | | | | | | | |
| | | | | | M99M99. | | N99N99.M99M99. | | | | | | | |
| | | | | | | | H99H99.W99W99. | | | | | | | |
| 590 | 8 | GQGDAFC | HAVSR | HYLNVSCSL | G99G99.Q99Q99. | H99H99.A87B00. | H99H99.Y99Y99. | 21 | 15 | 27 | 63 | Yes | Yes | Yes |
| | | | | | G99G99.D99D99. | V99V99.S99S99. | L99L99.N99N99. | | | | | | | |
| | | | | | A99A99.F99F99. | R99R99. | V99V99.S99S99. | | | | | | | |
| | | | | | C99C99. | | C99C99.S99S99. | | | | | | | |
| | | | | | | | L99L99. | | | | | | | |
| 599 | 8 | SLVNVVW | NVPDL | ATFTTFVVT | S99S99.L99L99. | N99N99.V99V99. | A99A99.T99T99. | 21 | 15 | 27 | 63 | Yes | Yes | Yes |
| | | | | | V99V99.N99N99. | P99P99.D99D99. | F99F99.T99T99. | | | | | | | |
| | | | | | V99V99.V99V99. | L99L99. | T99T99.F99F99. | | | | | | | |
| | | | | | W99W99. | | V99V99.V99V99. | | | | | | | |
| | | | | | | | T99T99. | | | | | | | |
| 646 | 8 | SVTVRA | LYSRG | HYSIPTEHCL | S87B00.V99V99. | L99L99.Y99Y99. | H99H99.Y99Y99. | 18 | 15 | 30 | 63 | Yes | Yes | Yes |
| | | | | | T99T99.V99V99. | S99S99.R99R99. | S99S99.I99I99. | | | | | | | |
| | | | | | R99R99.A99A99. | G99G99. | P99P99.T99T99. | | | | | | | |
| | | | | | | | E99E99.H99H99. | | | | | | | |
| | | | | | | | C99C99.L99L99. | | | | | | | |
| 649 | 8 | PYLGRLA | GYACA | IALSLSGFIL | P99P99.Y99Y99. | G99G99.Y99Y99. | I99I99.A99A99. | 21 | 15 | 30 | 66 | Yes | Yes | Yes |
| | | | | | L99L99.G99G99. | A99A99.C99C99. | L99L99.S99S99. | | | | | | | |
| | | | | | R87B00.L99L99. | A99A99. | L99L99.S99S99. | | | | | | | |
| | | | | | A99A99. | | G99G99.F99F99. | | | | | | | |
| | | | | | | | I99I99.L99L99. | | | | | | | |
| 652 | 8 | GRGCCVY | YDNVQ | ARYTAVAVV | G99G99.R99R99. | Y99Y99.D99D99. | A99A99.R99R99. | 21 | 15 | 27 | 63 | Yes | Yes | Yes |
| | | | | | G99G99.C99C99. | N99N99.V99V99. | Y99Y99.T99T99. | | | | | | | |
| | | | | | C99C99.V99V99. | Q99Q99. | A99A99.V99V99. | | | | | | | |
| | | | | | Y99Y99. | | A99A99.V99V99. | | | | | | | |
| | | | | | | | V99V99. | | | | | | | |
| 662 | 8 | PRCYCVW | PGGGE | PCWSHRLGVF | P99P99.R99R99. | P99P99.G99G99. | P99P99.C99C99. | 21 | 15 | 30 | 66 | Yes | Yes | Yes |
| | | | | | C99C99.Y99Y99. | G99G99.G99G99. | W99W99.S99S99. | | | | | | | |
| | | | | | C99C99.V99V99. | E99E99. | H99H99.R99R99. | | | | | | | |
| | | | | | W99W99. | | L99L99.G99G99. | | | | | | | |
| | | | | | | | V99V99.F99F99. | | | | | | | |
| 668 | 8 | IS | AHSSC | VHVSVHSGLVCIC | B00 | A99A99.H99H99. | V99V99.H99H99. | 0 | 15 | 39 | 54 | Yes | Yes | Yes |
| | | | | | | S99S99.S99S99. | V99V99.S99S99. | | | | | | | |
| | | | | | | C99C99. | V99V99.H99H99. | | | | | | | |
| | | | | | | | S99S99.G99G99. | | | | | | | |
| | | | | | | | L99L99.V99V99. | | | | | | | |
| | | | | | | | C99C99.I99I99. | | | | | | | |
| | | | | | | | C99C99. | | | | | | | |
| 669 | 8 | LSAVCF | HDYTA | IVFNPIGGVL | L99L99.S99S99. | H99H99.D99D99. | I99I99.V99V99. | 18 | 15 | 30 | 63 | Yes | Yes | Yes |
| | | | | | A99A99.V99V99. | Y99Y99.T99T99. | F99F99.N99N99. | | | | | | | |
| | | | | | C99C99.F99F99. | A99A99. | P99P99.I99I99. | | | | | | | |
| | | | | | | | G99G99.G99G99. | | | | | | | |
| | | | | | | | V99V99.L99L99. | | | | | | | |
| 674 | 8 | FSVVRCS | LHAFG | RIVFDEYHIA | F99F99.S99S99. | L99L99.H99H99. | R99R99.I99I99. | 21 | 15 | 30 | 66 | Yes | Yes | Yes |
| | | | | | V99V99.V99V99. | A99A99.F99F99. | V99V99.F99F99. | | | | | | | |
| | | | | | R99R99.C99C99. | G99G99. | D99D99.E99E99. | | | | | | | |
| | | | | | S99S99. | | Y99Y99.H99H99. | | | | | | | |
| | | | | | | | I99I99.A99A99. | | | | | | | |
| 680 | 8 | LSVLTQR | SHLGP | ATVLVPSYHCAGC | L99L99.S99S99. | S99S99.H99H99. | A99A99.T99T99. | 21 | 15 | 39 | 75 | Yes | Yes | Yes |
| | | | | | V99V99.L99L99. | L99L99.G99G99. | V99V99.L99L99. | | | | | | | |
| | | | | | T99T99.Q87B00. | P99P99. | V99V99.P99P99. | | | | | | | |
| | | | | | R99R99. | | S99S99.Y99Y99. | | | | | | | |
| | | | | | | | H99H99.C99C99. | | | | | | | |
| | | | | | | | A99A99.G99G99. | | | | | | | |
| | | | | | | | C99C99. | | | | | | | |
| 683 | 8 | ARSITL | PGDRF | FSWFHIDALL | A99A99.R99R99. | P99P99.G99G99. | F99F99.S99S99. | 18 | 15 | 30 | 63 | Yes | Yes | Yes |
| | | | | | S99S99.I99I99. | D99D99.R99R99. | W99W99.F99F99. | | | | | | | |
| | | | | | T99T99.L99L99. | F99F99. | H99H99.I99I99. | | | | | | | |
| | | | | | | | D99D99.A99A99. | | | | | | | |
| | | | | | | | L99L99.L99L99. | | | | | | | |
| 711 | 8 | FGVYYLG | RMC | HCFSFTRNNLPVE | F99F99.G99G99. | R99R99.M99M99. | H99H99.C99C99. | 21 | 9 | 39 | 69 | Yes | Yes | Yes |
| | | | | | V99V99.Y99Y99. | C99C99. | F99F99.S99S99. | | | | | | | |
| | | | | | Y99Y99.L99L99. | | F87B00.T99T99. | | | | | | | |
| | | | | | G99G99. | | R99R99.N99N99. | | | | | | | |
| | | | | | | | N99N99.L99L99. | | | | | | | |
| | | | | | | | P99P99.V99V99. | | | | | | | |
| | | | | | | | E99E99. | | | | | | | |
| 732 | 8 | TGVGSVF | RGGPG | AYMDIHTCVN | T87B00.G99G99. | R99R99.G99G99. | A99A99.Y99Y99. | 21 | 15 | 30 | 66 | Yes | Yes | Yes |
| | | | | | V99V99.G99G99. | G99G99.P99P99. | M99M99.D99D99. | | | | | | | |
| | | | | | S99S99.V99V99. | G99G99. | I99I99.H99H99. | | | | | | | |
| | | | | | F99F99. | | T99T99.C99C99. | | | | | | | |
| | | | | | | | V99V99.N99N99. | | | | | | | |
| 741 | 8 | RLTRTIG | SCLAD | TDLPAEFLCA | R99R99.L99L99. | S99S99.C99C99. | T99T99.D99D99. | 21 | 15 | 30 | 66 | Yes | Yes | Yes |
| | | | | | T87B00.R99R99. | L99L99.A99A99. | L99L99.P99P99. | | | | | | | |
| | | | | | T99T99.I99I99. | D99D99. | A99A99.E99E99. | | | | | | | |
| | | | | | G99G99. | | F99F99.L99L99. | | | | | | | |
| | | | | | | | C99C99.A99A99. | | | | | | | |
| 752 | 8 | SADVAPT | GFCWG | CFDGRF | S87B00.A99A99. | G99G99.F99F99. | C99C99.F99F99. | 21 | 15 | 18 | 54 | Yes | Yes | Yes |
| | | | | | D99D99.V99V99. | C99C99.W99W99. | D99D99.G99G99. | | | | | | | |
| | | | | | A99A99.P99P99. | G99G99. | R99R99.F99F99. | | | | | | | |
| | | | | | T99T99. | | | | | | | | | |
| 755 | 8 | MCDRFIW | FDNNF | VTPIW | M87B00.C99C99. | F99F99.D99D99. | V99V99.T99T99. | 21 | 15 | 15 | 51 | Yes | Yes | Yes |
| | | | | | D99D99.R99R99. | N99N99.N99N99. | P99P99.I99I99. | | | | | | | |
| | | | | | F99F99.I99I99. | F99F99. | W99W99. | | | | | | | |
| | | | | | W99W99. | | | | | | | | | |
| 768 | 8 | AGFLLIA | RAATP | TCLSDCHLCC | A99A99.G99G99. | R99R99.A87B00. | T99T99.C99C99. | 21 | 15 | 30 | 66 | Yes | Yes | Yes |
| | | | | | F99F99.L99L99. | A99A99.T99T99. | L99L99.S99S99. | | | | | | | |
| | | | | | L99L99.I99I99. | P99P99. | D99D99.C99C99. | | | | | | | |
| | | | | | A99A99. | | H99H99.L99L99. | | | | | | | |
| | | | | | | | C99C99.C99C99. | | | | | | | |
| 784 | 8 | IEMVPLG | GTTGW | MLWDMDACH | I99I99.E99E99. | G99G99.T99T99. | M75B00.L75B00. | 21 | 15 | 19 | 55 | Yes | Yes | Yes |
| | | | | | M99M99.V99V99. | T99T99.G99G99. | W75B00.D75B00. | | | | | | | |
| | | | | | P99P99.L99L99. | W99W99. | M75B00.D75B00. | | | | | | | |
| | | | | | G99G99. | | A75B00.C75B00. | | | | | | | |
| | | | | | | | H99H99. | | | | | | | |
| 785 | 8 | NGRVNVS | FGGLR | AFVLPHRENTDVL | N99N99.G99G99. | F99F99.G99G99. | A99A99.F99F99. | 21 | 15 | 39 | 75 | Yes | Yes | Yes |
| | | | | | R99R99.V99V99. | G99G99.L99L99. | V99V99.L99L99. | | | | | | | |
| | | | | | N99N99.V99V99. | R99R99. | P99P99.H99H99. | | | | | | | |
| | | | | | S99S99. | | R99R99.E99E99. | | | | | | | |
| | | | | | | | N99N99.T99T99. | | | | | | | |
| | | | | | | | D99D99.V99V99. | | | | | | | |
| | | | | | | | L99L99. | | | | | | | |
| 791 | 8 | VTSLIQH | LALLD | VTRFAY | V99V99.T99T99. | L99L99.A99A99. | V99V99.T99T99. | 21 | 15 | 18 | 54 | Yes | Yes | Yes |
| | | | | | S99S99.L99L99. | L99L99.L99L99. | R99R99.F99F99. | | | | | | | |
| | | | | | I99I99.Q99Q99. | D99D99. | A99A99.Y99Y99. | | | | | | | |
| | | | | | H99H99. | | | | | | | | | |
| 802 | 8 | SVASWCM | RSLLS | VIVVHSGSTSEAC | S99S99.V99V99. | R99R99.S99S99. | V99V99.I99I99. | 21 | 15 | 39 | 75 | Yes | Yes | Yes |
| | | | | | A99A99.S99S99. | L99L99.L99L99. | V99V99.V87B00. | | | | | | | |
| | | | | | W99W99.C99C99. | S99S99. | H99H99.S99S99. | | | | | | | |
| | | | | | M99M99. | | G99G99.S99S99. | | | | | | | |
| | | | | | | | T99T99.S99S99. | | | | | | | |
| | | | | | | | E99E99.A99A99. | | | | | | | |
| | | | | | | | C99C99. | | | | | | | |
| 806 | 8 | VVSHCVF | CARAG | AYLSIDDIIY | V99V99.V99V99. | C99C99.A99A99. | A99A99.Y99Y99. | 21 | 15 | 30 | 66 | Yes | Yes | Yes |
| | | | | | S99S99.H99H99. | R99R99.A99A99. | L99L99.S99S99. | | | | | | | |
| | | | | | C99C99.V99V99. | G99G99. | I99I99.D99D99. | | | | | | | |
| | | | | | F99F99. | | D99D99.I99I99. | | | | | | | |
| | | | | | | | I99I99.Y99Y99. | | | | | | | |
| 827 | 8 | CASNLLG | CLPCQ | FTWSSLGRR | C99C99.A99A99. | C99C99.L99L99. | F99F99.T99T99. | 21 | 15 | 27 | 63 | Yes | Yes | Yes |
| | | | | | S99S99.N99N99. | P99P99.C99C99. | W99W99.S99S99. | | | | | | | |
| | | | | | L99L99.L99L99. | Q99Q99. | S99S99.L99L99. | | | | | | | |
| | | | | | G99G99. | | G99G99.R99R99. | | | | | | | |
| | | | | | | | R99R99. | | | | | | | |
| 829 | 8 | VHHGMVF | LAGIE | GYLCGRDVS | V99V99.H99H99. | L99L99.A99A99. | G99G99.Y99Y99. | 21 | 15 | 27 | 63 | Yes | Yes | Yes |
| | | | | | H99H99.G99G99. | G99G99.I99I99. | L99L99.C99C99. | | | | | | | |
| | | | | | M99M99.V99V99. | E99E99. | G99G99.R99R99. | | | | | | | |
| | | | | | F99F99. | | D99D99.V99V99. | | | | | | | |
| | | | | | | | S99S99. | | | | | | | |
| 83 | 8 | SKGVMA | NMGIT | LILSNSILM | S87B00.K99K99. | N99N99.M99M99. | L99L99.I99I99. | 21 | 15 | 27 | 63 | Yes | Yes | Yes |
| | | | | | G99G99.V99V99. | G99G99.I99I99. | L99L99.S99S99. | | | | | | | |
| | | | | | S99S99.M99M99. | T99T99. | N99N99.S99S99. | | | | | | | |
| | | | | | A99A99. | | I99I99.L99L99. | | | | | | | |
| | | | | | | | M99M99. | | | | | | | |
| 839 | 8 | FGLGPGW | LPIGH | VSQVGG | F99F99.G99G99. | L99L99.P99P99. | V99V99.S99S99. | 21 | 15 | 18 | 54 | Yes | Yes | Yes |
| | | | | | L99L99.G99G99. | I99I99.G99G99. | Q99Q99.V99V99. | | | | | | | |
| | | | | | P99P99.G99G99. | H99H99. | G99G99.G99G99. | | | | | | | |
| | | | | | W99W99. | | | | | | | | | |
| 842 | 8 | RADSYIW | SCGRD | LYSWSDSCLF | R99R99.A99A99. | S99S99.C99C99. | L99L99.Y99Y99. | 21 | 15 | 30 | 66 | Yes | Yes | Yes |
| | | | | | D99D99.S99S99. | G99G99.R99R99. | S99S99.W99W99. | | | | | | | |
| | | | | | Y99Y99.I99I99. | D99D99. | S99S99.D99D99. | | | | | | | |
| | | | | | W99W99. | | S99S99.C99C99. | | | | | | | |
| | | | | | | | L99L99.F99F99. | | | | | | | |
| 844 | 8 | LLRYNTY | PVFGS | SMLGCS | L99L99.L99L99. | P99P99.V99V99. | S99S99.M99M99. | 21 | 15 | 18 | 54 | Yes | Yes | Yes |
| | | | | | R99R99.Y99Y99. | F99F99.G99G99. | L99L99.G99G99. | | | | | | | |
| | | | | | N99N99.T99T99. | S99S99. | C99C99.S99S99. | | | | | | | |
| | | | | | Y99Y99. | | | | | | | | | |
| 86 | 8 | RFVFSLY | AFDVD | VPPHYHELVK | R99R99.F99F99. | A99A99.F99F99. | V99V99.P99P99. | 21 | 15 | 30 | 66 | Yes | Yes | Yes |
| | | | | | V99V99.F99F99. | D99D99.V99V99. | P99P99.H99H99. | | | | | | | |
| | | | | | S99S99.L99L99. | D99D99. | Y99Y99.H99H99. | | | | | | | |
| | | | | | Y99Y99. | | E99E99.L99L99. | | | | | | | |
| | | | | | | | V99V99.K99K99. | | | | | | | |
| 863 | 8 | WIKWLHV | SMCGH | CPCCFS | W99W99.I87B00. | S99S99.M99M99. | B00 | 21 | 15 | 0 | 36 | Yes | Yes | Yes |
| | | | | | K99K99.W99W99. | C99C99.G99G99. | | | | | | | | |
| | | | | | L99L99.H99H99. | H99H99. | | | | | | | | |
| | | | | | V99V99. | | | | | | | | | |
| 874 | 8 | FLMERHT | VILAS | FPAIYPDWR | F99F99.L99L99. | V99V99.I99I99. | F99F99.P99P99. | 21 | 15 | 27 | 63 | Yes | Yes | Yes |
| | | | | | M99M99.E99E99. | L99L99.A99A99. | A99A99.I99I99. | | | | | | | |
| | | | | | R99R99.H99H99. | S99S99. | Y99Y99.P99P99. | | | | | | | |
| | | | | | T99T99. | | D99D99.W99W99. | | | | | | | |
| | | | | | | | R99R99. | | | | | | | |
| 880 | 8 | GSMIVTL | HGSST | AVLSLTNMTLLVQ | G99G99.S99S99. | H99H99.G99G99. | A99A99.V99V99. | 21 | 15 | 39 | 75 | Yes | Yes | Yes |
| | | | | | M99M99.I99I99. | S99S99.S99S99. | L99L99.S99S99. | | | | | | | |
| | | | | | V99V99.T99T99. | T99T99. | L99L99.T99T99. | | | | | | | |
| | | | | | L99L99. | | N99N99.M99M99. | | | | | | | |
| | | | | | | | T99T99.L99L99. | | | | | | | |
| | | | | | | | L99L99.V99V99. | | | | | | | |
| | | | | | | | Q99Q99. | | | | | | | |
| 883 | 8 | VFGAYNV | LLGTN | LGPMHHDGGL | V99V99.F99F99. | L99L99.L99L99. | L99L99.G99G99. | 21 | 15 | 30 | 66 | Yes | Yes | Yes |
| | | | | | G99G99.A99A99. | G99G99.T99T99. | P99P99.M99M99. | | | | | | | |
| | | | | | Y99Y99.N99N99. | N99N99. | H99H99.H99H99. | | | | | | | |
| | | | | | V99V99. | | D99D99.G99G99. | | | | | | | |
| | | | | | | | G99G99.L99L99. | | | | | | | |
| 890 | 8 | FIRRRVY | SASFS | YALADYGLVF | F99F99.I99I99. | S99S99.A99A99. | Y99Y99.A99A99. | 21 | 15 | 30 | 66 | Yes | Yes | Yes |
| | | | | | R99R99.R99R99. | S99S99.F99F99. | L99L99.A99A99. | | | | | | | |
| | | | | | R99R99.V99V99. | S99S99. | D99D99.Y99Y99. | | | | | | | |
| | | | | | Y99Y99. | | G99G99.L99L99. | | | | | | | |
| | | | | | | | V99V99.F99F99. | | | | | | | |
| 897 | 8 | LIRSVVW | RCGAV | YLYSHFSAVV | L99L99.I99I99. | R99R99.C99C99. | Y99Y99.L99L99. | 21 | 15 | 30 | 66 | Yes | Yes | Yes |
| | | | | | R99R99.S99S99. | G99G99.A99A99. | Y99Y99.S99S99. | | | | | | | |
| | | | | | V99V99.V99V99. | V99V99. | H99H99.F99F99. | | | | | | | |
| | | | | | W99W99. | | S99S99.A99A99. | | | | | | | |
| | | | | | | | V99V99.V99V99. | | | | | | | |
| 98 | 8 | ALCVFGG | LCGCC | AYYSKAALVGRVL | A99A99.L99L99. | L99L99.C99C99. | A99A99.Y99Y99. | 21 | 15 | 39 | 75 | Yes | Yes | Yes |
| | | | | | C99C99.V99V99. | G99G99.C99C99. | Y99Y99.S99S99. | | | | | | | |
| | | | | | F99F99.G99G99. | C99C99. | K99K99.A99A99. | | | | | | | |
| | | | | | G99G99. | | A99A99.L99L99. | | | | | | | |
| | | | | | | | V99V99.G99G99. | | | | | | | |
| | | | | | | | R99R99.V99V99. | | | | | | | |
| | | | | | | | L99L99. | | | | | | | |
| 128 | 7 | FVLCTRS | TGFIC | FHARSTMAGQDIY | F99F99.V99V99. | T99T99.G99G99. | F99F99.H99H99. | 21 | 15 | 39 | 75 | Yes | Yes | Yes |
| | | | | | L99L99.C99C99. | F99F99.I99I99. | A99A99.R99R99. | | | | | | | |
| | | | | | T99T99.R99R99. | C99C99. | S99S99.T99T99. | | | | | | | |
| | | | | | S99S99. | | M99M99.A99A99. | | | | | | | |
| | | | | | | | G99G99.Q99Q99. | | | | | | | |
| | | | | | | | D99D99.I99I99. | | | | | | | |
| | | | | | | | Y99Y99. | | | | | | | |
| 156 | 7 | HYLKVFG | MQLHG | AAYVSRSVD | H99H99.Y85B00. | M99M99.Q99Q99. | A99A99.A99A99. | 21 | 15 | 27 | 63 | Yes | Yes | Yes |
| | | | | | L99L99.K99K99. | L99L99.H99H99. | Y99Y99.V99V99. | | | | | | | |
| | | | | | V99V99.F99F99. | G99G99. | S99S99.R99R99. | | | | | | | |
| | | | | | G99G99. | | S99S99.V99V99. | | | | | | | |
| | | | | | | | D99D99. | | | | | | | |
| 166 | 7 | VVLTFTD | GPQFC | WPWTI | V99V99.V99V99. | G99G99.P99P99. | W99W99.P99P99. | 21 | 15 | 15 | 51 | Yes | Yes | Yes |
| | | | | | L99L99.T99T99. | Q99Q99.F99F99. | W99W99.T99T99. | | | | | | | |
| | | | | | F99F99.T99T99. | C99C99. | I99I99. | | | | | | | |
| | | | | | D99D99. | | | | | | | | | |
| 188 | 7 | LYSYLAI | SRVQY | SCSSDI | L99L99.Y99Y99. | S99S99.R99R99. | S99S99.C99C99. | 21 | 15 | 18 | 54 | Yes | Yes | Yes |
| | | | | | S99S99.Y99Y99. | V99V99.Q99Q99. | S99S99.S99S99. | | | | | | | |
| | | | | | L99L99.A99A99. | Y99Y99. | D99D99.I99I99. | | | | | | | |
| | | | | | I99I99. | | | | | | | | | |
| 250 | 7 | LVH | SYGGY | SNLEGT | L99L99.V99V99. | S99S99.Y99Y99. | S99S99.N99N99. | 9 | 15 | 18 | 42 | Yes | Yes | Yes |
| | | | | | H99H99. | G99G99.G99G99. | L99L99.E99E99. | | | | | | | |
| | | | | | | Y99Y99. | G99G99.T99T99. | | | | | | | |
| 265 | 7 | MVMRCVF | QSGVG | VHLASSLEFL | M99M99.V99V99. | Q99Q99.S99S99. | V99V99.H99H99. | 21 | 15 | 30 | 66 | Yes | Yes | Yes |
| | | | | | M99M99.R99R99. | G99G99.V99V99. | L99L99.A99A99. | | | | | | | |
| | | | | | C99C99.V99V99. | G99G99. | S99S99.S99S99. | | | | | | | |
| | | | | | F99F99. | | L99L99.E99E99. | | | | | | | |
| | | | | | | | F99F99.L99L99. | | | | | | | |
| 289 | 7 | MDI | RHTFV | LDSIY | M99M99.D99D99. | R99R99.H99H99. | L99L99.D99D99. | 9 | 15 | 15 | 39 | Yes | Yes | Yes |
| | | | | | I99I99. | T99T99.F99F99. | S99S99.I99I99. | | | | | | | |
| | | | | | | V99V99. | Y99Y99. | | | | | | | |
| 297 | 7 | GRAYYVC | RRDDD | YVYEESFRHPVVR | G99G99.R99R99. | R99R99.R99R99. | B00 | 21 | 15 | 0 | 36 | Yes | Yes | Yes |
| | | | | | A99A99.Y99Y99. | D99D99.D99D99. | | | | | | | | |
| | | | | | Y99Y99.V99V99. | D99D99. | | | | | | | | |
| | | | | | C99C99. | | | | | | | | | |
| 304 | 7 | DMSGVFI | AVSCC | ARFLSISARV | D99D99.M71B00. | A99A99.V99V99. | A99A99.R99R99. | 20 | 15 | 30 | 65 | Yes | Yes | Yes |
| | | | | | S85B00.G85B00. | S99S99.C99C99. | F99F99.L99L99. | | | | | | | |
| | | | | | V85B00.F85B00. | C99C99. | S99S99.I99I99. | | | | | | | |
| | | | | | I85B00. | | S99S99.A99A99. | | | | | | | |
| | | | | | | | R99R99.V99V99. | | | | | | | |
| 322 | 7 | YFKCLVY | NLDDY | VCLLRTVSPIHAV | Y99Y99.F99F99. | N99N99.L99L99. | V99V99.C99C99. | 21 | 15 | 39 | 75 | Yes | Yes | Yes |
| | | | | | K85B00.C99C99. | D99D99.D99D99. | L99L99.L99L99. | | | | | | | |
| | | | | | L99L99.V99V99. | Y99Y99. | R99R99.T99T99. | | | | | | | |
| | | | | | Y99Y99. | | V99V99.S99S99. | | | | | | | |
| | | | | | | | P99P99.I99I99. | | | | | | | |
| | | | | | | | H99H99.A99A99. | | | | | | | |
| | | | | | | | V99V99. | | | | | | | |
| 345 | 7 | LILRWSV | LRSCC | QYFIRDYPL | L99L99.I99I99. | L99L99.R99R99. | Q99Q99.Y99Y99. | 21 | 15 | 27 | 63 | Yes | Yes | Yes |
| | | | | | L99L99.R99R99. | S99S99.C99C99. | F99F99.I99I99. | | | | | | | |
| | | | | | W99W99.S99S99. | C99C99. | R99R99.D99D99. | | | | | | | |
| | | | | | V99V99. | | Y99Y99.P99P99. | | | | | | | |
| | | | | | | | L99L99. | | | | | | | |
| 354 | 7 | STHTLLA | CYQGA | HDIYSNDGLVVIG | S99S99.T99T99. | C99C99.Y99Y99. | H99H99.D99D99. | 21 | 15 | 39 | 75 | Yes | Yes | Yes |
| | | | | | H99H99.T99T99. | Q99Q99.G99G99. | I99I99.Y99Y99. | | | | | | | |
| | | | | | L99L99.L99L99. | A99A99. | S99S99.N99N99. | | | | | | | |
| | | | | | A99A99. | | D99D99.G99G99. | | | | | | | |
| | | | | | | | L99L99.V99V99. | | | | | | | |
| | | | | | | | V99V99.I99I99. | | | | | | | |
| | | | | | | | G99G99. | | | | | | | |
| 366 | 7 | HSIAQYY | TVQSR | FPAVNSESCF | H99H99.S99S99. | T99T99.V99V99. | F99F99.P99P99. | 21 | 15 | 30 | 66 | Yes | Yes | Yes |
| | | | | | I99I99.A99A99. | Q99Q99.S99S99. | A99A99.V99V99. | | | | | | | |
| | | | | | Q99Q99.Y99Y99. | R99R99. | N99N99.S99S99. | | | | | | | |
| | | | | | Y99Y99. | | E99E99.S99S99. | | | | | | | |
| | | | | | | | C99C99.F99F99. | | | | | | | |
| 376 | 7 | RLPCLVY | VDCTT | YTFVSVGCE | R99R99.L99L99. | V99V99.D99D99. | Y57I2.T71B00. | 21 | 15 | 17 | 53 | Yes | Yes | Yes |
| | | | | | P99P99.C99C99. | C99C99.T99T99. | F42B00.V57B00. | | | | | | | |
| | | | | | L99L99.V99V99. | T99T99. | S57B00.V57B00. | | | | | | | |
| | | | | | Y99Y99. | | G57B00.C57B00. | | | | | | | |
| | | | | | | | E57B00. | | | | | | | |
| 419 | 7 | VMGIVSG | LDAYR | ITRWSM | V99V99.M99M99. | L99L99.D99D99. | I99I99.T99T99. | 21 | 15 | 18 | 54 | Yes | Yes | Yes |
| | | | | | G99G99.I99I99. | A99A99.Y99Y99. | R99R99.W99W99. | | | | | | | |
| | | | | | V99V99.S99S99. | R99R99. | S99S99.M99M99. | | | | | | | |
| | | | | | G99G99. | | | | | | | | | |
| 43 | 7 | WPRRPLG | MWSGS | HCPDPNFSPVLIV | W99W99.P99P99. | M99M99.W99W99. | H99H99.C99C99. | 21 | 15 | 39 | 75 | Yes | Yes | Yes |
| | | | | | R99R99.R99R99. | S99S99.G99G99. | P99P99.D99D99. | | | | | | | |
| | | | | | P99P99.L99L99. | S99S99. | P99P99.N99N99. | | | | | | | |
| | | | | | G99G99. | | F99F99.S99S99. | | | | | | | |
| | | | | | | | P99P99.V99V99. | | | | | | | |
| | | | | | | | L99L99.I99I99. | | | | | | | |
| | | | | | | | V99V99. | | | | | | | |
| 431 | 7 | PSRPVVY | HDGLI | VFFVNGSGTL | P99P99.S99S99. | H99H99.D99D99. | V99V99.F99F99. | 21 | 15 | 30 | 66 | Yes | Yes | Yes |
| | | | | | R99R99.P99P99. | G99G99.L99L99. | F99F99.V99V99. | | | | | | | |
| | | | | | V99V99.V99V99. | I99I99. | N99N99.G99G99. | | | | | | | |
| | | | | | Y99Y99. | | S99S99.G99G99. | | | | | | | |
| | | | | | | | T99T99.L99L99. | | | | | | | |
| 454 | 7 | WGSSPVS | QYAGG | WYSCLQ | W99W99.G99G99. | Q99Q99.Y99Y99. | W99W99.Y99Y99. | 21 | 15 | 18 | 54 | Yes | Yes | Yes |
| | | | | | S85B00.S99S99. | A99A99.G99G99. | S99S99.C99C99. | | | | | | | |
| | | | | | P99P99.V99V99. | G99G99. | L99L99.Q99Q99. | | | | | | | |
| | | | | | S99S99. | | | | | | | | | |
| 455 | 7 | CWRHMFV | CDSGN | GILCYDGFV | C99C99.W99W99. | C99C99.D99D99. | G99G99.I99I99. | 21 | 15 | 27 | 63 | Yes | Yes | Yes |
| | | | | | R99R99.H99H99. | S99S99.G99G99. | L99L99.C99C99. | | | | | | | |
| | | | | | M99M99.F99F99. | N99N99. | Y99Y99.D99D99. | | | | | | | |
| | | | | | V99V99. | | G99G99.F85B00. | | | | | | | |
| | | | | | | | V85B00. | | | | | | | |
| 469 | 7 | YVGSNVY | TGHPN | AFFFVYDVS | Y99Y99.V99V99. | T99T99.G99G99. | A99A99.F99F99. | 21 | 15 | 27 | 63 | Yes | Yes | Yes |
| | | | | | G99G99.S99S99. | H99H99.P99P99. | F99F99.F99F99. | | | | | | | |
| | | | | | N99N99.V99V99. | N99N99. | V99V99.Y99Y99. | | | | | | | |
| | | | | | Y99Y99. | | D99D99.V99V99. | | | | | | | |
| | | | | | | | S99S99. | | | | | | | |
| 481 | 7 | GFNVVLW | GSRLI | VLAFKCHGGFECC | G99G99.F99F99. | G99G99.S99S99. | V99V99.L99L99. | 21 | 15 | 39 | 75 | Yes | Yes | Yes |
| | | | | | N99N99.V99V99. | R99R99.L99L99. | A99A99.F99F99. | | | | | | | |
| | | | | | V99V99.L99L99. | I99I99. | K99K99.C99C99. | | | | | | | |
| | | | | | W99W99. | | H99H99.G99G99. | | | | | | | |
| | | | | | | | G99G99.F99F99. | | | | | | | |
| | | | | | | | E99E99.C99C99. | | | | | | | |
| | | | | | | | C99C99. | | | | | | | |
| 497 | 7 | EYMRIMI | TWHMH | QLLQPTFHVT | E99E99.Y99Y99. | T99T99.W99W99. | Q99Q99.L99L99. | 21 | 15 | 30 | 66 | Yes | Yes | Yes |
| | | | | | M99M99.R99R99. | H99H99.M99M99. | L99L99.Q99Q99. | | | | | | | |
| | | | | | I99I99.M99M99. | H99H99. | P99P99.T99T99. | | | | | | | |
| | | | | | I99I99. | | F99F99.H99H99. | | | | | | | |
| | | | | | | | V99V99.T99T99. | | | | | | | |
| 500 | 7 | YFERIYC | IVACS | ATGSTA | Y99Y99.F99F99. | I99I99.V99V99. | A99A99.T99T99. | 21 | 15 | 18 | 54 | Yes | Yes | Yes |
| | | | | | E99E99.R99R99. | A99A99.C99C99. | G99G99.S99S99. | | | | | | | |
| | | | | | I99I99.Y99Y99. | S99S99. | T99T99.A99A99. | | | | | | | |
| | | | | | C99C99. | | | | | | | | | |
| 527 | 7 | LRSSWWGR | GGPFT | SGPDTTLFRL | L99L99.R99R99. | G99G99.G99G99. | S99S99.G99G99. | 21 | 15 | 30 | 66 | Yes | Yes | Yes |
| | | | | | S99S99.S99S99. | P99P99.F99F99. | P99P99.D99D99. | | | | | | | |
| | | | | | W99W99.G99G99. | T99T99. | T99T99.T99T99. | | | | | | | |
| | | | | | R99R99. | | L99L99.F99F99. | | | | | | | |
| | | | | | | | R99R99.L99L99. | | | | | | | |
| 528 | 7 | PDTRVVY | TWPHD | TFLCSCLGSL | P99P99.D99D99. | T99T99.W99W99. | T99T99.F99F99. | 21 | 15 | 30 | 66 | Yes | Yes | Yes |
| | | | | | T99T99.R99R99. | P99P99.H99H99. | L99L99.C99C99. | | | | | | | |
| | | | | | V99V99.V99V99. | D99D99. | S99S99.C99C99. | | | | | | | |
| | | | | | Y99Y99. | | L99L99.G99G99. | | | | | | | |
| | | | | | | | S99S99.L99L99. | | | | | | | |
| 538 | 7 | VCIVCGN | NYTYS | RSFNEDADFY | V99V99.C99C99. | N99N99.Y99Y99. | R99R99.S99S99. | 21 | 15 | 30 | 66 | Yes | Yes | Yes |
| | | | | | I99I99.V99V99. | T99T99.Y99Y99. | F99F99.N99N99. | | | | | | | |
| | | | | | C99C99.G99G99. | S99S99. | E99E99.D99D99. | | | | | | | |
| | | | | | N99N99. | | A99A99.D99D99. | | | | | | | |
| | | | | | | | F99F99.Y99Y99. | | | | | | | |
| 541 | 7 | ASRTCGF | EFSFH | LRFEPVAVW | A99A99.S99S99. | E99E99.F99F99. | L99L99.R99R99. | 21 | 15 | 27 | 63 | Yes | Yes | Yes |
| | | | | | R99R99.T99T99. | S99S99.F99F99. | F99F99.E99E99. | | | | | | | |
| | | | | | C99C99.G99G99. | H99H99. | P99P99.V99V99. | | | | | | | |
| | | | | | F99F99. | | A99A99.V99V99. | | | | | | | |
| | | | | | | | W99W99. | | | | | | | |
| 556 | 7 | KGCFWHA | VHKEL | DLLTHAVGLCRHE | K99K99.G99G99. | V99V99.H99H99. | D99D99.L99L99. | 21 | 15 | 39 | 75 | Yes | Yes | Yes |
| | | | | | C99C99.F99F99. | K99K99.E99E99. | L99L99.T99T99. | | | | | | | |
| | | | | | W99W99.H85B00. | L99L99. | H99H99.A99A99. | | | | | | | |
| | | | | | A99A99. | | V99V99.G99G99. | | | | | | | |
| | | | | | | | L99L99.C99C99. | | | | | | | |
| | | | | | | | R99R99.H99H99. | | | | | | | |
| | | | | | | | E99E99. | | | | | | | |
| 561 | 7 | YTNNYVF | AVGQI | ARFGDHHMVH | Y99Y99.T99T99. | A99A99.V99V99. | A99A99.R99R99. | 21 | 15 | 30 | 66 | Yes | Yes | Yes |
| | | | | | N99N99.N99N99. | G99G99.Q99Q99. | F99F99.G99G99. | | | | | | | |
| | | | | | Y99Y99.V99V99. | I99I99. | D99D99.H99H99. | | | | | | | |
| | | | | | F85B00. | | H99H99.M99M99. | | | | | | | |
| | | | | | | | V99V99.H99H99. | | | | | | | |
| 562 | 7 | IGGLTLV | WVHCV | SSYKDGGIV | I99I99.G99G99. | W99W99.V99V99. | S99S99.S99S99. | 21 | 15 | 27 | 63 | Yes | Yes | Yes |
| | | | | | G99G99.L99L99. | H99H99.C99C99. | Y99Y99.K99K99. | | | | | | | |
| | | | | | T99T99.L99L99. | V99V99. | D99D99.G99G99. | | | | | | | |
| | | | | | V99V99. | | G99G99.I99I99. | | | | | | | |
| | | | | | | | V99V99. | | | | | | | |
| 565 | 7 | GRNPIVW | RDRVD | VHLKHVGLLQ | G99G99.R99R99. | R99R99.D99D99. | V99V99.H99H99. | 21 | 15 | 30 | 66 | Yes | Yes | Yes |
| | | | | | N99N99.P99P99. | R99R99.V99V99. | L99L99.K99K99. | | | | | | | |
| | | | | | I99I99.V99V99. | D99D99. | H99H99.V99V99. | | | | | | | |
| | | | | | W99W99. | | G99G99.L99L99. | | | | | | | |
| | | | | | | | L99L99.Q99Q99. | | | | | | | |
| 566 | 7 | FMRGSLY | CAGRP | VTFSSDGPV | F99F99.M99M99. | C99C99.A99A99. | V99V99.T99T99. | 21 | 15 | 27 | 63 | Yes | Yes | Yes |
| | | | | | R99R99.G99G99. | G99G99.R99R99. | F99F99.S99S99. | | | | | | | |
| | | | | | S99S99.L99L99. | P99P99. | S99S99.D99D99. | | | | | | | |
| | | | | | Y99Y99. | | G99G99.P99P99. | | | | | | | |
| | | | | | | | V99V99. | | | | | | | |
| 573 | 7 | NYGGVYT | RAPDL | NWWLDEWKSNDVS | N99N99.Y99Y99. | R99R99.A99A99. | N99N99.W99W99. | 21 | 15 | 39 | 75 | Yes | Yes | Yes |
| | | | | | G99G99.G99G99. | P99P99.D99D99. | W99W99.L99L99. | | | | | | | |
| | | | | | V99V99.Y99Y99. | L99L99. | D99D99.E99E99. | | | | | | | |
| | | | | | T99T99. | | W99W99.K99K99. | | | | | | | |
| | | | | | | | S99S99.N99N99. | | | | | | | |
| | | | | | | | D99D99.V99V99. | | | | | | | |
| | | | | | | | S99S99. | | | | | | | |
| 596 | 7 | VSGIIRI | KRSYT | RSLFDH | V85B00.S99S99. | K99K99.R99R99. | R99R99.S99S99. | 21 | 15 | 18 | 54 | Yes | Yes | Yes |
| | | | | | G85B00.I85B00. | S99S99.Y99Y99. | L99L99.F99F99. | | | | | | | |
| | | | | | I85B00.R85B00. | T99T99. | D85B00.H99H99. | | | | | | | |
| | | | | | I85B00. | | | | | | | | | |
| 603 | 7 | VSFCERF | VHSFR | FDLTTPWLPA | V99V99.S99S99. | V99V99.H99H99. | B00 | 21 | 15 | 0 | 36 | Yes | Yes | Yes |
| | | | | | F99F99.C99C99. | S99S99.F99F99. | | | | | | | | |
| | | | | | E99E99.R99R99. | R99R99. | | | | | | | | |
| | | | | | F99F99. | | | | | | | | | |
| 615 | 7 | TTYNRVH | GAYNG | HHYFHGTVF | T99T99.T99T99. | G99G99.A99A99. | H99H99.H99H99. | 21 | 15 | 27 | 63 | Yes | Yes | Yes |
| | | | | | Y99Y99.N99N99. | Y99Y99.N99N99. | Y99Y99.F99F99. | | | | | | | |
| | | | | | R99R99.V99V99. | G99G99. | H99H99.G99G99. | | | | | | | |
| | | | | | H99H99. | | T99T99.V99V99. | | | | | | | |
| | | | | | | | F99F99. | | | | | | | |
| 623 | 7 | FYCGYVS | YSHAC | AYSHCGPSEG | F99F99.Y99Y99. | Y99Y99.S99S99. | B00 | 21 | 15 | 0 | 36 | Yes | Yes | Yes |
| | | | | | C99C99.G85B00. | H99H99.A99A99. | | | | | | | | |
| | | | | | Y99Y99.V99V99. | C99C99. | | | | | | | | |
| | | | | | S99S99. | | | | | | | | | |
| 633 | 7 | EYMGLLG | GFFKL | HMYNGFDSII | E99E99.Y99Y99. | G99G99.F99F99. | H99H99.M99M99. | 21 | 15 | 30 | 66 | Yes | Yes | Yes |
| | | | | | M99M99.G99G99. | F99F99.K99K99. | Y99Y99.N99N99. | | | | | | | |
| | | | | | L99L99.L99L99. | L99L99. | G99G99.F99F99. | | | | | | | |
| | | | | | G99G99. | | D99D99.S99S99. | | | | | | | |
| | | | | | | | I99I99.I99I99. | | | | | | | |
| 640 | 7 | SGCYPPF | TVSYI | TTFGEAGRK | S85B00.G99G99. | T85B00.V85B00. | T71B00.T99T99. | 21 | 15 | 19 | 55 | Yes | Yes | Yes |
| | | | | | C85B00.Y99Y99. | S85B00.Y85B00. | F71B00.G71B00. | | | | | | | |
| | | | | | P85B00.P85B00. | I85B00. | E71B00.A71B00. | | | | | | | |
| | | | | | F85B00. | | G71B00.R71B00. | | | | | | | |
| | | | | | | | K71B00. | | | | | | | |
| 663 | 7 | NACVVFR | LSSRW | YAVHAILSDSDVT | N99N99.A99A99. | L99L99.S99S99. | Y99Y99.A99A99. | 21 | 15 | 39 | 75 | Yes | Yes | Yes |
| | | | | | C99C99.V99V99. | S99S99.R99R99. | V99V99.H99H99. | | | | | | | |
| | | | | | V99V99.F99F99. | W99W99. | A99A99.I99I99. | | | | | | | |
| | | | | | R99R99. | | L99L99.S99S99. | | | | | | | |
| | | | | | | | D99D99.S99S99. | | | | | | | |
| | | | | | | | D99D99.V99V99. | | | | | | | |
| | | | | | | | T99T99. | | | | | | | |
| 666 | 7 | VRISIWV | TWRER | LYGPFA | V99V99.R99R99. | T99T99.W99W99. | L99L99.Y99Y99. | 21 | 15 | 18 | 54 | Yes | Yes | Yes |
| | | | | | I99I99.S99S99. | R99R99.E99E99. | G99G99.P99P99. | | | | | | | |
| | | | | | I99I99.W99W99. | R99R99. | F99F99.A99A99. | | | | | | | |
| | | | | | V99V99. | | | | | | | | | |
| 673 | 7 | RVIGLWA | GLERR | YLYSALWGSI | R99R99.V99V99. | G99G99.L99L99. | Y99Y99.L99L99. | 21 | 15 | 30 | 66 | Yes | Yes | Yes |
| | | | | | I99I99.G99G99. | E99E99.R99R99. | Y99Y99.S99S99. | | | | | | | |
| | | | | | L99L99.W99W99. | R85B00. | A99A99.L99L99. | | | | | | | |
| | | | | | A99A99. | | W99W99.G99G99. | | | | | | | |
| | | | | | | | S99S99.I99I99. | | | | | | | |
| 684 | 7 | TCLHCLG | SSFSF | WMEADGECF | T71B00.C99C99. | S85B00.S99S99. | W99W99.M99M99. | 20 | 15 | 27 | 62 | Yes | Yes | Yes |
| | | | | | L99L99.H99H99. | F99F99.S99S99. | E99E99.A99A99. | | | | | | | |
| | | | | | C99C99.L99L99. | F99F99. | D99D99.G99G99. | | | | | | | |
| | | | | | G99G99. | | E99E99.C99C99. | | | | | | | |
| | | | | | | | F99F99. | | | | | | | |
| 696 | 7 | LDCFYYL | IGWGR | LHGERKPTPP | L99L99.D99D99. | I99I99.G99G99. | B00 | 21 | 15 | 0 | 36 | Yes | Yes | Yes |
| | | | | | C99C99.F99F99. | W99W99.G99G99. | | | | | | | | |
| | | | | | Y99Y99.Y99Y99. | R99R99. | | | | | | | | |
| | | | | | L99L99. | | | | | | | | | |
| 701 | 7 | KMHRVIW | CVPAG | GYLWCTYVL | K99K99.M99M99. | C99C99.V99V99. | G99G99.Y99Y99. | 21 | 15 | 27 | 63 | Yes | Yes | Yes |
| | | | | | H99H99.R99R99. | P99P99.A99A99. | L99L99.W99W99. | | | | | | | |
| | | | | | V99V99.I99I99. | G99G99. | C99C99.T99T99. | | | | | | | |
| | | | | | W99W99. | | Y99Y99.V99V99. | | | | | | | |
| | | | | | | | L99L99. | | | | | | | |
| 702 | 7 | ACYSGYV | AFTNN | FPSATTAWGY | A99A99.C85B00. | A99A99.F99F99. | F99F99.P99P99. | 21 | 15 | 30 | 66 | Yes | Yes | Yes |
| | | | | | Y99Y99.S99S99. | T99T99.N99N99. | S99S99.A99A99. | | | | | | | |
| | | | | | G99G99.Y99Y99. | N99N99. | T99T99.T99T99. | | | | | | | |
| | | | | | V99V99. | | A99A99.W99W99. | | | | | | | |
| | | | | | | | G99G99.Y99Y99. | | | | | | | |
| 72 | 7 | VYMAYCR | GIVGW | YIDAYSPTES | V99V99.Y99Y99. | G99G99.I99I99. | Y99Y99.I99I99. | 21 | 15 | 30 | 66 | Yes | Yes | Yes |
| | | | | | M99M99.A99A99. | V99V99.G99G99. | D99D99.A99A99. | | | | | | | |
| | | | | | Y99Y99.C99C99. | W99W99. | Y99Y99.S99S99. | | | | | | | |
| | | | | | R99R99. | | P99P99.T99T99. | | | | | | | |
| | | | | | | | E99E99.S99S99. | | | | | | | |
| 724 | 7 | AHHQSVI | DEDRA | YNFVARAGLTSIC | A85B00.H99H99. | D99D99.E99E99. | Y99Y99.N99N99. | 21 | 15 | 39 | 75 | Yes | Yes | Yes |
| | | | | | H99H99.Q99Q99. | D99D99.R85B00. | F85B00.V99V99. | | | | | | | |
| | | | | | S99S99.V99V99. | A99A99. | A99A99.R99R99. | | | | | | | |
| | | | | | I85B00. | | A99A99.G99G99. | | | | | | | |
| | | | | | | | L99L99.T99T99. | | | | | | | |
| | | | | | | | S99S99.I99I99. | | | | | | | |
| | | | | | | | C99C99. | | | | | | | |
| 729 | 7 | ERCSWSS | RCYRI | TIPSPAFGGD | E99E99.R99R99. | R99R99.C99C99. | T99T99.I99I99. | 21 | 15 | 30 | 66 | Yes | Yes | Yes |
| | | | | | C99C99.S99S99. | Y99Y99.R99R99. | P99P99.S99S99. | | | | | | | |
| | | | | | W99W99.S99S99. | I99I99. | P99P99.A99A99. | | | | | | | |
| | | | | | S99S99. | | F99F99.G99G99. | | | | | | | |
| | | | | | | | G99G99.D99D99. | | | | | | | |
| 736 | 7 | LRVRVMA | SYLCE | TVLDLAFAGSYTF | L99L99.R99R99. | S99S99.Y99Y99. | T99T99.V99V99. | 21 | 15 | 39 | 75 | Yes | Yes | Yes |
| | | | | | V99V99.R99R99. | L99L99.C99C99. | L99L99.D99D99. | | | | | | | |
| | | | | | V99V99.M99M99. | E99E99. | L99L99.A99A99. | | | | | | | |
| | | | | | A99A99. | | F99F99.A99A99. | | | | | | | |
| | | | | | | | G99G99.S99S99. | | | | | | | |
| | | | | | | | Y99Y99.T99T99. | | | | | | | |
| | | | | | | | F99F99. | | | | | | | |
| 756 | 7 | VPTCNVY | RIGST | AYWLNHSTVT | V99V99.P99P99. | R99R99.I99I99. | A99A99.Y99Y99. | 21 | 15 | 30 | 66 | Yes | Yes | Yes |
| | | | | | T99T99.C99C99. | G99G99.S99S99. | W99W99.L99L99. | | | | | | | |
| | | | | | N99N99.V99V99. | T99T99. | N99N99.H99H99. | | | | | | | |
| | | | | | Y99Y99. | | S99S99.T99T99. | | | | | | | |
| | | | | | | | V99V99.T99T99. | | | | | | | |
| 761 | 7 | GCMDMFV | FFGFH | AEDIIRVGVIPWV | G99G99.C99C99. | F99F99.F99F99. | A99A99.E99E99. | 21 | 15 | 39 | 75 | Yes | Yes | Yes |
| | | | | | M99M99.D99D99. | G99G99.F99F99. | D99D99.I99I99. | | | | | | | |
| | | | | | M99M99.F99F99. | H99H99. | I99I99.R99R99. | | | | | | | |
| | | | | | V99V99. | | V99V99.G99G99. | | | | | | | |
| | | | | | | | V99V99.I99I99. | | | | | | | |
| | | | | | | | P99P99.W99W99. | | | | | | | |
| | | | | | | | V99V99. | | | | | | | |
| 763 | 7 | RCSRCCY | TDMRH | SYLCDGLDVK | R99R99.C99C99. | T99T99.D99D99. | S99S99.Y99Y99. | 21 | 15 | 30 | 66 | Yes | Yes | Yes |
| | | | | | S99S99.R99R99. | M99M99.R99R99. | L99L99.C99C99. | | | | | | | |
| | | | | | C99C99.C99C99. | H99H99. | D99D99.G99G99. | | | | | | | |
| | | | | | Y99Y99. | | L99L99.D99D99. | | | | | | | |
| | | | | | | | V99V99.K99K99. | | | | | | | |
| 770 | 7 | NFVVVFG | FGSRE | HATDLRDSQLCIR | N99N99.F99F99. | F99F99.G99G99. | H99H99.A99A99. | 21 | 15 | 39 | 75 | Yes | Yes | Yes |
| | | | | | V99V99.V99V99. | S99S99.R99R99. | T99T99.D99D99. | | | | | | | |
| | | | | | V99V99.F99F99. | E99E99. | L99L99.R99R99. | | | | | | | |
| | | | | | G99G99. | | D99D99.S99S99. | | | | | | | |
| | | | | | | | Q99Q99.L99L99. | | | | | | | |
| | | | | | | | C99C99.I99I99. | | | | | | | |
| | | | | | | | R99R99. | | | | | | | |
| 778 | 7 | WSVKVFD | AHAGG | HRLTDVGLV | W99W99.S99S99. | A99A99.H99H99. | H99H99.R99R99. | 21 | 15 | 27 | 63 | Yes | Yes | Yes |
| | | | | | V99V99.K99K99. | A99A99.G99G99. | L99L99.T99T99. | | | | | | | |
| | | | | | V99V99.F99F99. | G99G99. | D99D99.V99V99. | | | | | | | |
| | | | | | D99D99. | | G99G99.L99L99. | | | | | | | |
| | | | | | | | V99V99. | | | | | | | |
| 780 | 7 | TLSLRSH | SFIFN | HAIHADSAELSVR | T99T99.L99L99. | S99S99.F99F99. | H99H99.A99A99. | 21 | 15 | 39 | 75 | Yes | Yes | Yes |
| | | | | | S99S99.L99L99. | I99I99.F99F99. | I99I99.H99H99. | | | | | | | |
| | | | | | R99R99.S99S99. | N99N99. | A99A99.D85B00. | | | | | | | |
| | | | | | H99H99. | | S99S99.A99A99. | | | | | | | |
| | | | | | | | E99E99.L99L99. | | | | | | | |
| | | | | | | | S99S99.V99V99. | | | | | | | |
| | | | | | | | R99R99. | | | | | | | |
| 781 | 7 | LPVPC | FFNGR | FATVSS | L99L99.P99P99. | F99F99.F99F99. | F99F99.A99A99. | 15 | 15 | 18 | 48 | Yes | Yes | Yes |
| | | | | | V99V99.P99P99. | N99N99.G99G99. | T99T99.V99V99. | | | | | | | |
| | | | | | C99C99. | R99R99. | S99S99.S99S99. | | | | | | | |
| 783 | 7 | GLHFVYS | FRADS | YCMAYDAVL | G99G99.L99L99. | F99F99.R99R99. | Y99Y99.C99C99. | 21 | 15 | 27 | 63 | Yes | Yes | Yes |
| | | | | | H99H99.F99F99. | A99A99.D99D99. | M99M99.A99A99. | | | | | | | |
| | | | | | V99V99.Y99Y99. | S99S99. | Y99Y99.D99D99. | | | | | | | |
| | | | | | S99S99. | | A99A99.V99V99. | | | | | | | |
| | | | | | | | L99L99. | | | | | | | |
| 792 | 7 | CFSAVGS | LWMVR | WLSCAG | C99C99.F99F99. | L99L99.W99W99. | W99W99.L99L99. | 21 | 15 | 18 | 54 | Yes | Yes | Yes |
| | | | | | S85B00.A99A99. | M99M99.V99V99. | S99S99.C99C99. | | | | | | | |
| | | | | | V99V99.G99G99. | R99R99. | A99A99.G99G99. | | | | | | | |
| | | | | | S99S99. | | | | | | | | | |
| 794 | 7 | LGVVLVQ | GGLVD | GAVWPF | L99L99.G99G99. | G99G99.G99G99. | G99G99.A99A99. | 21 | 15 | 18 | 54 | Yes | Yes | Yes |
| | | | | | V99V99.V99V99. | L99L99.V99V99. | V99V99.W99W99. | | | | | | | |
| | | | | | L99L99.V99V99. | D99D99. | P99P99.F99F99. | | | | | | | |
| | | | | | Q99Q99. | | | | | | | | | |
| 799 | 7 | PYE | NRGRG | IFLYTQKVSE | P99P99.Y99Y99. | N99N99.R99R99. | I99I99.F99F99. | 9 | 15 | 30 | 54 | Yes | Yes | Yes |
| | | | | | E99E99. | G99G99.R85B00. | L99L99.Y99Y99. | | | | | | | |
| | | | | | | G99G99. | T99T99.Q99Q99. | | | | | | | |
| | | | | | | | K99K99.V99V99. | | | | | | | |
| | | | | | | | S99S99.E99E99. | | | | | | | |
| 816 | 7 | YLFCTMA | LGAR | ELTNGL | Y99Y99.L99L99. | L99L99.G99G99. | E99E99.L99L99. | 21 | 12 | 18 | 5 | Yes | Yes | Yes |
| | | | | | F99F99.C99C99. | A99A99.R99R99. | T99T99.N99N99. | | | | | | | |
| | | | | | T99T99.M99M99. | | G99G99.L99L99. | | | | | | | |
| | | | | | A99A99. | | | | | | | | | |
| 825 | 7 | FTERVVS | YGARG | VMALFMAGLTLS | F99F99.T99T99. | Y99Y99.G99G99. | B00 | 21 | 15 | 0 | 36 | Yes | Yes | Yes |
| | | | | | E99E99.R99R99. | A99A99.R99R99. | | | | | | | | |
| | | | | | V99V99.V99V99. | G99G99. | | | | | | | | |
| | | | | | S99S99. | | | | | | | | | |
| 832 | 7 | VSEVPYA | TITTN | IMLRRTYVCY | V99V99.S99S99. | T99T99.I99I99. | I99I99.M99M99. | 21 | 15 | 30 | 66 | Yes | Yes | Yes |
| | | | | | E99E99.V99V99. | T99T99.T99T99. | L99L99.R99R99. | | | | | | | |
| | | | | | P99P99.Y99Y99. | N99N99. | R99R99.T99T99. | | | | | | | |
| | | | | | A99A99. | | Y99Y99.V99V99. | | | | | | | |
| | | | | | | | C99C99.Y99Y99. | | | | | | | |
| 84 | 7 | YSDTWYL | RCLAQ | KGLSGVSVT | Y99Y99.S99S99. | R99R99.C99C99. | K99K99.G99G99. | 21 | 15 | 27 | 63 | Yes | Yes | Yes |
| | | | | | D99D99.T99T99. | L99L99.A99A99. | L99L99.S99S99. | | | | | | | |
| | | | | | W99W99.Y99Y99. | Q99Q99. | G99G99.V99V99. | | | | | | | |
| | | | | | L99L99. | | S99S99.V99V99. | | | | | | | |
| | | | | | | | T99T99. | | | | | | | |
| 841 | 7 | GVNGNVY | ANDII | AYITLSSDVV | G99G99.V99V99. | A99A99.N99N99. | A99A99.Y99Y99. | 21 | 15 | 30 | 99 | Yes | Yes | Yes |
| | | | | | N85B00.G99G99. | D99D99.I99I99. | I99I99.T99T99. | | | | | | | |
| | | | | | N99N99.V99V99. | I99I99. | L99L99.S99S99. | | | | | | | |
| | | | | | Y99Y99. | | S99S99.D99D99. | | | | | | | |
| | | | | | | | V99V99.V99V99. | | | | | | | |
| 846 | 7 | CCINRFT | WYANF | PVPDDNVMFF | C99C99.C99C99. | W99W99.Y99Y99. | P99P99.V99V99. | 21 | 15 | 30 | 66 | Yes | Yes | Yes |
| | | | | | I99I99.N99N99. | A99A99.N99N99. | P99P99.D99D99. | | | | | | | |
| | | | | | R99R99.F99F99. | F99F99. | D99D99.N99N99. | | | | | | | |
| | | | | | T99T99. | | V99V99.M99M99. | | | | | | | |
| | | | | | | | F99F99.F99F99. | | | | | | | |
| 854 | 7 | FNVHVFG | LDTIR | AILNEYQYRIGVT | F85B00.N85B00. | L85B00.D85B00. | A99A99.I99I99. | 21 | 15 | 39 | 75 | Yes | Yes | Yes |
| | | | | | V85B00.H85B00. | T85B00.I85B00. | L99L99.N99N99. | | | | | | | |
| | | | | | V85B00.F85B00. | R85B00. | E99E99.Y99Y99. | | | | | | | |
| | | | | | G85B00. | | Q99Q99.Y99Y99. | | | | | | | |
| | | | | | | | R99R99.I99I99. | | | | | | | |
| | | | | | | | G99G99.V99V99. | | | | | | | |
| | | | | | | | T99T99. | | | | | | | |
| 861 | 7 | CIHSCVF | YASVG | CVYIPHGGVN | C99C99.I99I99. | Y99Y99.A99A99. | B00 | 21 | 15 | 0 | 36 | Yes | Yes | Yes |
| | | | | | H99H99.S99S99. | S99S99.V99V99. | | | | | | | | |
| | | | | | C99C99.V99V99. | G99G99. | | | | | | | | |
| | | | | | F99F99. | | | | | | | | | |
| 876 | 7 | SGATPMS | LPAIY | LFDQHDGSVL | S99S99.G99G99. | L99L99.P99P99. | L85B00.F85B00. | 21 | 15 | 29 | 65 | Yes | Yes | Yes |
| | | | | | A99A99.T99T99. | A99A99.I99I99. | D85B00.Q85B00. | | | | | | | |
| | | | | | P99P99.M99M99. | Y99Y99. | H85B00.D71B00. | | | | | | | |
| | | | | | S99S99. | | G85B00.S85B00. | | | | | | | |
| | | | | | | | V85B00.L85B00. | | | | | | | |
| 884 | 7 | PTCGQLS | RPFGD | VYWSQRVVI | P99P99.T99T99. | R99R99.P99P99. | V99V99.Y99Y99. | 21 | 15 | 27 | 63 | Yes | Yes | Yes |
| | | | | | C99C99.G99G99. | F99F99.G99G99. | W99W99.S99S99. | | | | | | | |
| | | | | | Q99Q99.L99L99. | D99D99. | Q99Q99.R99R99. | | | | | | | |
| | | | | | S85B00. | | V99V99.V99V99. | | | | | | | |
| | | | | | | | I99I99. | | | | | | | |
| 885 | 7 | VIGILEG | RVFCV | KSRCSPQFSC | V99V99.I99I99. | R99R99.V99V99. | K99K99.S99S99. | 21 | 15 | 30 | 66 | Yes | Yes | Yes |
| | | | | | G99G99.I99I99. | F99F99.C99C99. | R99R99.C99C99. | | | | | | | |
| | | | | | L99L99.E99E99. | V99V99. | S99S99.P99P99. | | | | | | | |
| | | | | | G99G99. | | Q99Q99.F99F99. | | | | | | | |
| | | | | | | | S99S99.C99C99. | | | | | | | |
| 893 | 7 | GVSDVFV | SCSGW | ASAHYQNIIL | G99G99.V99V99. | S99S99.C99C99. | A99A99.S99S99. | 21 | 15 | 30 | 66 | Yes | Yes | Yes |
| | | | | | S99S99.D99D99. | S99S99.G99G99. | A99A99.H99H99. | | | | | | | |
| | | | | | V99V99.F99F99. | W99W99. | Y99Y99.Q99Q99. | | | | | | | |
| | | | | | V99V99. | | N99N99.I99I99. | | | | | | | |
| | | | | | | | I99I99.L99L99. | | | | | | | |
| 894 | 7 | KTTSAVY | CSVYD | VYYCRLSSVN | K99K99.T99T99. | C99C99.S99S99. | V99V99.Y99Y99. | 21 | 15 | 30 | 66 | Yes | Yes | Yes |
| | | | | | T99T99.S99S99. | V99V99.Y99Y99. | Y99Y99.C99C99. | | | | | | | |
| | | | | | A99A99.V99V99. | D99D99. | R99R99.L99L99. | | | | | | | |
| | | | | | Y99Y99. | | S99S99.S99S99. | | | | | | | |
| | | | | | | | V99V99.N99N99. | | | | | | | |
| 901 | 7 | NGGKSVW | TCGCY | PYFSSGAVF | N99N99.G99G99. | T99T99.C99C99. | P99P99.Y99Y99. | 21 | 15 | 27 | 63 | Yes | Yes | Yes |
| | | | | | G99G99.K99K99. | G99G99.C99C99. | F99F99.S99S99. | | | | | | | |
| | | | | | S99S99.V99V99. | Y99Y99. | S99S99.G99G99. | | | | | | | |
| | | | | | W99W99. | | A99A99.V99V99. | | | | | | | |
| | | | | | | | F99F99. | | | | | | | |
| 909 | 7 | NACDVCQ | CSHRL | IDSSS | N99N99.A99A99. | C99C99.S99S99. | I99I99.D99D99. | 21 | 15 | 15 | 51 | Yes | Yes | Yes |
| | | | | | C99C99.D99D99. | H99H99.R99R99. | S99S99.S99S99. | | | | | | | |
| | | | | | V99V99.C99C99. | L99L99. | S99S99. | | | | | | | |
| | | | | | Q99Q99. | | | | | | | | | |
| 911 | 7 | LVSRMSE | YTRSR | TALVAQGYHYDAT | L99L99.V99V99. | Y99Y99.T99T99. | T99T99.A99A99. | 21 | 15 | 39 | 75 | Yes | Yes | Yes |
| | | | | | S99S99.R99R99. | R99R99.S99S99. | L99L99.V99V99. | | | | | | | |
| | | | | | M99M99.S99S99. | R99R99. | A99A99.Q99Q99. | | | | | | | |
| | | | | | E99E99. | | G99G99.Y99Y99. | | | | | | | |
| | | | | | | | H99H99.Y99Y99. | | | | | | | |
| | | | | | | | D99D99.A99A99. | | | | | | | |
| | | | | | | | T99T99. | | | | | | | |
| 915 | 7 | FGSI | HNYQL | HIRTRSSDGFWYE | F99F99.G99G99. | H99H99.N99N99. | H99H99.I99I99. | 12 | 15 | 39 | 66 | Yes | Yes | Yes |
| | | | | | S99S99.I99I99. | Y99Y99.Q99Q99. | R99R99.T99T99. | | | | | | | |
| | | | | | | L99L99. | R99R99.S99S99. | | | | | | | |
| | | | | | | | S99S99.D99D99. | | | | | | | |
| | | | | | | | G99G99.F99F99. | | | | | | | |
| | | | | | | | W99W99.Y99Y99. | | | | | | | |
| | | | | | | | E99E99. | | | | | | | |
| 920 | 7 | A | LDCGR | SHIHLPLNRWVVT | A99A99. | L99L99.D99D99. | S85B00.H99H99. | 3 | 15 | 39 | 57 | Yes | Yes | Yes |
| | | | | | | C99C99.G99G99. | I99I99.H99H99. | | | | | | | |
| | | | | | | R85B00. | L99L99.P99P99. | | | | | | | |
| | | | | | | | L99L99.N99N99. | | | | | | | |
| | | | | | | | R99R99.W99W99. | | | | | | | |
| | | | | | | | V99V99.V99V99. | | | | | | | |
| | | | | | | | T99T99. | | | | | | | |
| 113 | 6 | PLYCAV | SGGVG | ACLTSTGFLSYVK | P99P99.L99L99. | S99S99.G99G99. | A99A99.C99C99. | 21 | 15 | 39 | 75 | Yes | Yes | Yes |
| | | | | | Y99Y99.C99C99. | G99G99.V99V99. | L99L99.T99T99. | | | | | | | |
| | | | | | A99A99.V99V99. | G99G99. | S99S99.T99T99. | | | | | | | |
| | | | | | Y99Y99. | | G99G99.F99F99. | | | | | | | |
| | | | | | | | L99L99.S99S99. | | | | | | | |
| | | | | | | | Y99Y99.V99V99. | | | | | | | |
| | | | | | | | K99K99. | | | | | | | |
| 132 | 6 | PIDRLVF | VDSMS | VAFFGN | P99P99.I99I99. | V99V99.D99D99. | B00 | 21 | 15 | 0 | 36 | Yes | Yes | Yes |
| | | | | | D99D99.R99R99. | S99S99.M99M99. | | | | | | | | |
| | | | | | L99L99.V99V99. | S99S99. | | | | | | | | |
| | | | | | F99F99. | | | | | | | | | |
| 169 | 6 | EINFCLF | TGSQE | SLYVFPSAHR | E99E99.I99I99. | T99T99.G99G99. | S99S99.L99L99. | 21 | 15 | 30 | 66 | Yes | Yes | Yes |
| | | | | | N99N99.F99F99. | S99S99.Q99Q99. | Y99Y99.V99V99. | | | | | | | |
| | | | | | C99C99.L99L99. | E99E99. | F99F99.P99P99. | | | | | | | |
| | | | | | F99F99. | | S99S99.A99A99. | | | | | | | |
| | | | | | | | H99H99.R99R99. | | | | | | | |
| 269 | 6 | LYCGCVF | VDRQL | AIMRNVGLDYEIN | L99L99.Y99Y99. | V99V99.D99D99. | B00 | 21 | 15 | 0 | 36 | Yes | Yes | Yes |
| | | | | | C99C99.G99G99. | R99R99.Q99Q99. | | | | | | | | |
| | | | | | C99C99.V99V99. | L99L99. | | | | | | | | |
| | | | | | F99F99. | | | | | | | | | |
| 282 | 6 | RSSRRVY | VNSWD | VCYEPSVGAV | R99R99.S99S99. | V99V99.N99N99. | V99V99.C99C99. | 21 | 15 | 30 | 66 | Yes | Yes | Yes |
| | | | | | S99S99.R99R99. | S99S99.W99W99. | Y99Y99.E99E99. | | | | | | | |
| | | | | | R99R99.V99V99. | D99D99. | P99P99.S99S99. | | | | | | | |
| | | | | | Y99Y99. | | V99V99.G99G99. | | | | | | | |
| | | | | | | | A99A99.V99V99. | | | | | | | |
| 296 | 6 | GVHLVAI | LFEGV | LCVAPTRLAW | G99G99.V99V99. | L99L99.F99F99. | L99L99.C99C99. | 21 | 15 | 30 | 66 | Yes | Yes | Yes |
| | | | | | H99H99.L99L99. | E99E99.G99G99. | V99V99.A99A99. | | | | | | | |
| | | | | | V99V99.A99A99. | V99V99. | P99P99.T99T99. | | | | | | | |
| | | | | | I99I99. | | R99R99.L99L99. | | | | | | | |
| | | | | | | | A99A99.W99W99. | | | | | | | |
| 320 | 6 | QATRCPV | VSAQI | PTRMLTGLDI | Q99Q99.A99A99. | V99V99.S99S99. | P99P99.T99T99. | 21 | 15 | 30 | 66 | Yes | Yes | Yes |
| | | | | | T99T99.R99R99. | A99A99.Q99Q99. | R99R99.M99M99. | | | | | | | |
| | | | | | C99C99.P99P99. | I99I99. | L99L99.T99T99. | | | | | | | |
| | | | | | V99V99. | | G99G99.L99L99. | | | | | | | |
| | | | | | | | D99D99.I99I99. | | | | | | | |
| 329 | 6 | TWMWCLR | WQPAR | QVLTTHNAQV | T99T99.W99W99. | W99W99.Q99Q99. | Q99Q99.V99V99. | 21 | 15 | 30 | 66 | Yes | Yes | Yes |
| | | | | | M99M99.W99W99. | P99P99.A99A99. | L99L99.T99T99. | | | | | | | |
| | | | | | C99C99.L99L99. | R99R99. | T99T99.H99H99. | | | | | | | |
| | | | | | R99R99. | | N99N99.A99A99. | | | | | | | |
| | | | | | | | Q99Q99.V99V99. | | | | | | | |
| 343 | 6 | DCHRRVW | CGAAT | AHFNAGVIC | D99D99.C99C99. | C99C99.G99G99. | A99A99.H99H99. | 21 | 15 | 27 | 63 | Yes | Yes | Yes |
| | | | | | H99H99.R99R99. | A99A99.A99A99. | F99F99.N99N99. | | | | | | | |
| | | | | | R99R99.V99V99. | T99T99. | A99A99.G99G99. | | | | | | | |
| | | | | | W99W99. | | V99V99.I99I99. | | | | | | | |
| | | | | | | | C83B00. | | | | | | | |
| 355 | 6 | SGRPHVR | LNSDW | WGHG | S99S99.G99G99. | L99L99.N99N99. | W99W99.G99G99. | 21 | 15 | 12 | 48 | Yes | Yes | Yes |
| | | | | | R99R99.P99P99. | S99S99.D99D99. | H99H99.G99G99. | | | | | | | |
| | | | | | H99H99.V99V99. | W99W99. | | | | | | | | |
| | | | | | R99R99. | | | | | | | | | |
| 368 | 6 | IYNAYSC | YSLRV | ESKSGHCVGVEVS | I99I99.Y99Y99. | Y99Y99.S99S99. | E99E99.S99S99. | 21 | 15 | 39 | 75 | Yes | Yes | Yes |
| | | | | | N99N99.A99A99. | L99L99.R99R99. | K99K99.S99S99. | | | | | | | |
| | | | | | Y99Y99.S99S99. | V99V99. | G99G99.H99H99. | | | | | | | |
| | | | | | C99C99. | | C99C99.V99V99. | | | | | | | |
| | | | | | | | G99G99.V99V99. | | | | | | | |
| | | | | | | | E99E99.V99V99. | | | | | | | |
| | | | | | | | S99S99. | | | | | | | |
| 383 | 6 | VWVLGQR | HSGRD | HTVQIVCFSIILL | V99V99.W99W99. | H99H99.S99S99. | H99H99.T99T99. | 21 | 15 | 39 | 75 | Yes | Yes | Yes |
| | | | | | V99V99.L99L99. | G99G99.R99R99. | V99V99.Q99Q99. | | | | | | | |
| | | | | | G99G99.Q99Q99. | D99D99. | I99I99.V99V99. | | | | | | | |
| | | | | | R99R99. | | C99C99.F99F99. | | | | | | | |
| | | | | | | | S99S99.I99I99. | | | | | | | |
| | | | | | | | I99I99.L99L99. | | | | | | | |
| | | | | | | | L99L99. | | | | | | | |
| 406 | 6 | RDVERIV | TLATR | TWVLSQ | R99R99.D99D99. | T99T99.L99L99. | T99T99.W99W99. | 21 | 15 | 18 | 54 | Yes | Yes | Yes |
| | | | | | V99V99.E99E99. | A99A99.T99T99. | V99V99.L99L99. | | | | | | | |
| | | | | | R99R99.I99I99. | R99R99. | S99S99.Q99Q99. | | | | | | | |
| | | | | | V99V99. | | | | | | | | | |
| 412 | 6 | GLPNCVF | SDSSR | VLYVRNSWPL | G99G99.L99L99. | S99S99.D99D99. | V99V99.L99L99. | 21 | 15 | 30 | 66 | Yes | Yes | Yes |
| | | | | | P99P99.N99N99. | S99S99.S99S99. | Y99Y99.V99V99. | | | | | | | |
| | | | | | C99C99.V99V99. | R99R99. | R99R99.N99N99. | | | | | | | |
| | | | | | F99F99. | | S99S99.W99W99. | | | | | | | |
| | | | | | | | P99P99.L99L99. | | | | | | | |
| 415 | 6 | VLGAMGH | LVSGT | VDDWF | V99V99.L99L99. | L99L99.V99V99. | V99V99.D99D99. | 21 | 15 | 15 | 51 | Yes | Yes | Yes |
| | | | | | G99G99.A99A99. | S99S99.G83B00. | D99D99.W99W99. | | | | | | | |
| | | | | | M99M99.G99G99. | T99T99. | F99F99. | | | | | | | |
| | | | | | H99H99. | | | | | | | | | |
| 416 | 6 | WGRIPFV | LGGEE | PFTIAAFGPY | W99W99.G99G99. | L99L99.G99G99. | P99P99.F99F99. | 21 | 15 | 30 | 66 | Yes | Yes | Yes |
| | | | | | R99R99.I99I99. | G99G99.E99E99. | T99T99.I99I99. | | | | | | | |
| | | | | | P99P99.F99F99. | E99E99. | A99A99.A99A99. | | | | | | | |
| | | | | | V99V99. | | F99F99.G99G99. | | | | | | | |
| | | | | | | | P99P99.Y99Y99. | | | | | | | |
| 420 | 6 | SWNTPLA | PESLC | HLWMHAPVGF | S99S99.W99W99. | P99P99.E99E99. | H99H99.L99L99. | 21 | 15 | 30 | 66 | Yes | Yes | Yes |
| | | | | | N99N99.T99T99. | S99S99.L99L99. | W99W99.M99M99. | | | | | | | |
| | | | | | P99P99.L99L99. | C99C99. | H99H99.A99A99. | | | | | | | |
| | | | | | A99A99. | | P99P99.V99V99. | | | | | | | |
| | | | | | | | G99G99.F99F99. | | | | | | | |
| 422 | 6 | SMVGLLQ | NTALI | LVVTTSGHVG | S99S99.M99M99. | N99N99.T99T99. | L99L99.V99V99. | 21 | 15 | 30 | 66 | Yes | Yes | Yes |
| | | | | | V99V99.G99G99. | A99A99.L99L99. | V99V99.T99T99. | | | | | | | |
| | | | | | L99L99.L99L99. | I99I99. | T99T99.S99S99. | | | | | | | |
| | | | | | Q99Q99. | | G99G99.H99H99. | | | | | | | |
| | | | | | | | V99V99.G99G99. | | | | | | | |
| 458 | 6 | SPAHLLN | FCFGW | TLVPANDFPC | S99S99.P99P99. | F99F99.C99C99. | T99T99.L99L99. | 21 | 15 | 30 | 66 | Yes | Yes | Yes |
| | | | | | A99A99.H99H99. | F99F99.G99G99. | V99V99.P99P99. | | | | | | | |
| | | | | | L99L99.L99L99. | W99W99. | A99A99.N83B00. | | | | | | | |
| | | | | | N99N99. | | D99D99.F99F99. | | | | | | | |
| | | | | | | | P99P99.C99C99. | | | | | | | |
| 467 | 6 | SRA | GFGNC | PCYCI | S99S99.R99R99. | G99G99.F99F99. | B00 | 9 | 15 | 0 | 24 | Yes | Yes | Yes |
| | | | | | A99A99. | G99G99.N99N99. | | | | | | | | |
| | | | | | | C99C99. | | | | | | | | |
| 473 | 6 | LPFSVHS | VVFSL | TTPIGDYHYSLGP | L99L99.P99P99. | V99V99.V99V99. | T99T99.T99T99. | 21 | 15 | 39 | 75 | Yes | Yes | Yes |
| | | | | | F99F99.S99S99. | F99F99.S99S99. | P99P99.I99I99. | | | | | | | |
| | | | | | V99V99.H99H99. | L99L99. | G99G99.D99D99. | | | | | | | |
| | | | | | S99S99. | | Y99Y99.H99H99. | | | | | | | |
| | | | | | | | Y99Y99.S99S99. | | | | | | | |
| | | | | | | | L99L99.G99G99. | | | | | | | |
| | | | | | | | P99P99. | | | | | | | |
| 484 | 6 | LMTRVVH | GPRGS | ARFHANSVR | L99L99.M99M99. | G99G99.P99P99. | A99A99.R99R99. | 21 | 15 | 27 | 63 | Yes | Yes | Yes |
| | | | | | T99T99.R99R99. | R99R99.G99G99. | F99F99.H99H99. | | | | | | | |
| | | | | | V99V99.V99V99. | S99S99. | A83B00.N99N99. | | | | | | | |
| | | | | | H99H99. | | S99S99.V99V99. | | | | | | | |
| | | | | | | | R99R99. | | | | | | | |
| 489 | 6 | WRNWVLT | TAHSH | EYDEAK | W99W99.R99R99. | T99T99.A83B00. | E99E99.Y99Y99. | 21 | 15 | 18 | 54 | Yes | Yes | Yes |
| | | | | | N99N99.W99W99. | H99H99.S99S99. | D99D99.E99E99. | | | | | | | |
| | | | | | V99V99.L99L99. | H99H99. | A99A99.K99K99. | | | | | | | |
| | | | | | T99T99. | | | | | | | | | |
| 502 | 6 | LFSSVVF | CAGGL | VTASMSENLMLCR | L99L99.F99F99. | C99C99.A99A99. | V99V99.T99T99. | 21 | 15 | 39 | 75 | Yes | Yes | Yes |
| | | | | | S99S99.S99S99. | G99G99.G99G99. | A99A99.S99S99. | | | | | | | |
| | | | | | V99V99.V99V99. | L99L99. | M99M99.S99S99. | | | | | | | |
| | | | | | F99F99. | | E99E99.N99N99. | | | | | | | |
| | | | | | | | L99L99.M99M99. | | | | | | | |
| | | | | | | | L99L99.C99C99. | | | | | | | |
| | | | | | | | R99R99. | | | | | | | |
| 507 | 6 | LRRVDTS | TYEPG | RNSCSAIYGESCV | L99L99.R99R99. | T99T99.Y99Y99. | R99R99.N99N99. | 21 | 15 | 39 | 75 | Yes | Yes | Yes |
| | | | | | R99R99.V99V99. | E99E99.P83B00. | S99S99.C99C99. | | | | | | | |
| | | | | | D99D99.T99T99. | G99G99. | S99S99.A99A99. | | | | | | | |
| | | | | | S99S99. | | I99I99.Y99Y99. | | | | | | | |
| | | | | | | | G99G99.E99E99. | | | | | | | |
| | | | | | | | S99S99.C99C99. | | | | | | | |
| | | | | | | | V99V99. | | | | | | | |
| 521 | 6 | VYGILSS | VYGGC | YNRWSG | V99V99.Y99Y99. | V99V99.Y99Y99. | Y99Y99.N99N99. | 21 | 15 | 18 | 54 | Yes | Yes | Yes |
| | | | | | G99G99.I99I99. | G99G99.G99G99. | R99R99.W99W99. | | | | | | | |
| | | | | | L99L99.S99S99. | C99C99. | S83B00.G99G99. | | | | | | | |
| | | | | | S99S99. | | | | | | | | | |
| 526 | 6 | CRVEYSF | CVRGD | FRD | C99C99.R99R99. | C99C99.V99V99. | F99F99.R99R99. | 21 | 15 | 9 | 45 | Yes | Yes | Yes |
| | | | | | V99V99.E99E99. | R99R99.G99G99. | D83B00. | | | | | | | |
| | | | | | Y99Y99.S99S99. | D99D99. | | | | | | | | |
| | | | | | F99F99. | | | | | | | | | |
| 552 | 6 | STPDGFF | WFVED | YWISCVGYV | S99S99.T99T99. | W99W99.F99F99. | Y99Y99.W99W99. | 21 | 15 | 27 | 63 | Yes | Yes | Yes |
| | | | | | P99P99.D99D99. | V99V99.E99E99. | I99I99.S99S99. | | | | | | | |
| | | | | | G99G99.F99F99. | D99D99. | C99C99.V99V99. | | | | | | | |
| | | | | | F99F99. | | G99G99.Y99Y99. | | | | | | | |
| | | | | | | | V99V99. | | | | | | | |
| 570 | 6 | VKNFSAR | TDSVI | LYDWT | V99V99.K99K99. | T99T99.D99D99. | B00 | 21 | 15 | 0 | 36 | Yes | Yes | Yes |
| | | | | | N99N99.F99F99. | S99S99.V99V99. | | | | | | | | |
| | | | | | S99S99.A99A99. | I99I99. | | | | | | | | |
| | | | | | R99R99. | | | | | | | | | |
| 571 | 6 | ISVDRHQ | DVAGI | FSFSLMSRS | I99I99.S99S99. | D99D99.V99V99. | F99F99.S99S99. | 21 | 15 | 27 | 63 | Yes | Yes | Yes |
| | | | | | V99V99.D99D99. | A99A99.G99G99. | F99F99.S99S99. | | | | | | | |
| | | | | | R99R99.H99H99. | I99I99. | L99L99.M99M99. | | | | | | | |
| | | | | | Q99Q99. | | S99S99.R99R99. | | | | | | | |
| | | | | | | | S99S99. | | | | | | | |
| 574 | 6 | LGTVLLR | MHCNH | LLTCTALGSR | L99L99.G99G99. | M99M99.H99H99. | L99L99.L99L99. | 21 | 15 | 30 | 66 | Yes | Yes | Yes |
| | | | | | T99T99.V99V99. | C99C99.N99N99. | T99T99.C99C99. | | | | | | | |
| | | | | | L83B00.L99L99. | H99H99. | T99T99.A99A99. | | | | | | | |
| | | | | | R99R99. | | L99L99.G99G99. | | | | | | | |
| | | | | | | | S99S99.R99R99. | | | | | | | |
| 577 | 6 | FW | HPVSV | INFADGRRR | F99F99.W99W99. | H99H99.P99P99. | I99I99.N99N99. | 6 | 15 | 27 | 48 | Yes | Yes | Yes |
| | | | | | | V99V99.S99S99. | F99F99.A99A99. | | | | | | | |
| | | | | | | V99V99. | D99D99.G99G99. | | | | | | | |
| | | | | | | | R99R99.R99R99. | | | | | | | |
| | | | | | | | R99R99. | | | | | | | |
| 600 | 6 | RGSYTVVCRAV | MHNDYFNM | LTAYKDRCTNIY | R99R99.G99G99. | M99M99.H99H99. | L99L99.T99T99. | 21 | 15 | 27 | 63 | Yes | Yes | Yes |
| | | | | SVWSDAV | S99S99.Y99Y99. | N99N99.D99D99. | A99A99.Y99Y99. | | | | | | | |
| | | | | | T99T99.V99V99. | Y99Y99. | K99K99.D99D99. | | | | | | | |
| | | | | | V99V99. | | R99R99.C99C99. | | | | | | | |
| | | | | | | | T99T99. | | | | | | | |
| 602 | 6 | VCG | FMNSC | NIYSDAVCHGVWS | C99C99.R99R99. | F99F99.M99M99. | N99N99.I99I99. | 21 | 15 | 39 | 75 | Yes | Yes | Yes |
| | | | | | V99V99.C99C99. | N99N99.S99S99. | Y99Y99.S99S99. | | | | | | | |
| | | | | | G99G99.A99A99. | C99C99. | D99D99.A99A99. | | | | | | | |
| | | | | | V99V99. | | V99V99.C99C99. | | | | | | | |
| | | | | | | | H99H99.G99G99. | | | | | | | |
| | | | | | | | V99V99.W99W99. | | | | | | | |
| | | | | | | | S99S99. | | | | | | | |
| 607 | 6 | RVWCCYT | SGSWR | LAAFEYGAL | R99R99.V99V99. | S99S99.G99G99. | L99L99.A99A99. | 21 | 15 | 27 | 63 | Yes | Yes | Yes |
| | | | | | W99W99.C99C99. | S99S99.W99W99. | A99A99.F99F99. | | | | | | | |
| | | | | | C99C99.Y99Y99. | R99R99. | E99E99.Y99Y99. | | | | | | | |
| | | | | | T99T99. | | G99G99.A99A99. | | | | | | | |
| | | | | | | | L99L99. | | | | | | | |
| 622 | 6 | CLEHSYQ | GEFFR | FHHRDA | C99C99.L99L99. | G99G99.E99E99. | F66B00.H66B00. | 21 | 15 | 12 | 48 | Yes | Yes | Yes |
| | | | | | E99E99.H99H99. | F99F99.F99F99. | H66B00.R66B00. | | | | | | | |
| | | | | | S99S99.Y99Y99. | R99R99. | D66B00.A66B00. | | | | | | | |
| | | | | | Q99Q99. | | | | | | | | | |
| 625 | 6 | YCYTLLA | WSSSF | HFFASRSSIPTVQ | Y99Y99.C99C99. | W99W99.S99S99. | H99H99.F99F99. | 21 | 15 | 39 | 75 | Yes | Yes | Yes |
| | | | | | Y99Y99.T99T99. | S99S99.S99S99. | F99F99.A99A99. | | | | | | | |
| | | | | | L99L99.L99L99. | F99F99. | S99S99.R99R99. | | | | | | | |
| | | | | | A99A99. | | S99S99.S83B00. | | | | | | | |
| | | | | | | | I99I99.P99P99. | | | | | | | |
| | | | | | | | T99T99.V99V99. | | | | | | | |
| | | | | | | | Q99Q99. | | | | | | | |
| 637 | 6 | FQ | RHNLV | GHIFVH | F99F99.Q99Q99. | R99R99.H99H99. | G83B00.H99H99. | 6 | 15 | 18 | 39 | Yes | Yes | Yes |
| | | | | | | N99N99.L99L99. | I99I99.F83B00. | | | | | | | |
| | | | | | | V99V99. | V99V99.H99H99. | | | | | | | |
| 638 | 6 | PVARYWS | DVSHA | HNWGCLGRQ | P99P99.V99V99. | D99D99.V99V99. | H99H99.N99N99. | 21 | 15 | 27 | 63 | Yes | Yes | Yes |
| | | | | | A99A99.R99R99. | S99S99.H83B00. | W99W99.G99G99. | | | | | | | |
| | | | | | Y99Y99.W99W99. | A99A99. | C99C99.L99L99. | | | | | | | |
| | | | | | S99S99. | | G99G99.R99R99. | | | | | | | |
| | | | | | | | Q99Q99. | | | | | | | |
| 639 | 6 | LWVVYAK | SNLRL | MGATVY | L99L99.W99W99. | S99S99.N99N99. | M99M99.G99G99. | 21 | 15 | 18 | 54 | Yes | Yes | Yes |
| | | | | | V99V99.V99V99. | L99L99.R99R99. | A99A99.T99T99. | | | | | | | |
| | | | | | Y99Y99.A99A99. | L99L99. | V99V99.Y99Y99. | | | | | | | |
| | | | | | K99K99. | | | | | | | | | |
| 642 | 6 | FVFCQFL | EDGHG | H | F99F99.V99V99. | E99E99.D99D99. | H99H99. | 21 | 15 | 3 | 39 | Yes | Yes | Yes |
| | | | | | F99F99.C99C99. | G99G99.H99H99. | | | | | | | | |
| | | | | | Q99Q99.F99F99. | G99G99. | | | | | | | | |
| | | | | | L99L99. | | | | | | | | | |
| 650 | 6 | RLCGLVW | TSARD | IGSPYGH | R99R99.L99L99. | T99T99.S99S99. | I99I99.G99G99. | 21 | 15 | 21 | 57 | Yes | Yes | Yes |
| | | | | | C99C99.G99G99. | A99A99.R99R99. | S83B00.P99P99. | | | | | | | |
| | | | | | L99L99.V99V99. | D99D99. | Y99Y99.G99G99. | | | | | | | |
| | | | | | W99W99. | | H99H99. | | | | | | | |
| 651 | 6 | ELGRSVY | SCGTA | ATSNYSSSIV | E99E99.L99L99. | S99S99.C99C99. | A99A99.T99T99. | 21 | 15 | 30 | 66 | Yes | Yes | Yes |
| | | | | | G99G99.R99R99. | G99G99.T99T99. | S99S99.N99N99. | | | | | | | |
| | | | | | S99S99.V99V99. | A99A99. | Y99Y99.S99S99. | | | | | | | |
| | | | | | Y99Y99. | | S99S99.S99S99. | | | | | | | |
| | | | | | | | I99I99.V99V99. | | | | | | | |
| 658 | 6 | KRTDFQL | VGIEI | LTFVTRDTRL | K99K99.R99R99. | V99V99.G99G99. | L99L99.T99T99. | 21 | 15 | 30 | 66 | Yes | Yes | Yes |
| | | | | | T99T99.D99D99. | I99I99.E99E99. | F99F99.V99V99. | | | | | | | |
| | | | | | F99F99.Q99Q99. | I99I99. | T99T99.R99R99. | | | | | | | |
| | | | | | L99L99. | | D99D99.T99T99. | | | | | | | |
| | | | | | | | R99R99.L99L99. | | | | | | | |
| 66 | 6 | HRCMA | RGSRD | WIHRDTLYCE | H99H99.R99R99. | R99R99.G99G99. | W99W99.I99I99. | 15 | 15 | 30 | 60 | Yes | Yes | Yes |
| | | | | | C99C99.M99M99. | S99S99.R99R99. | H99H99.R99R99. | | | | | | | |
| | | | | | A99A99. | D99D99. | D99D99.T99T99. | | | | | | | |
| | | | | | | | L99L99.Y99Y99. | | | | | | | |
| | | | | | | | C99C99.E99E99. | | | | | | | |
| 660 | 6 | SLVRLVF | HRDPT | ANINQNHGVF | S99S99.L99L99. | H99H99.R99R99. | A99A99.N99N99. | 21 | 15 | 30 | 66 | Yes | Yes | Yes |
| | | | | | V99V99.R99R99. | D99D99.P99P99. | I99I99.N99N99. | | | | | | | |
| | | | | | L99L99.V99V99. | T99T99. | Q99Q99.N99N99. | | | | | | | |
| | | | | | F99F99. | | H99H99.G99G99. | | | | | | | |
| | | | | | | | V99V99.F99F99. | | | | | | | |
| 667 | 6 | CRANEVV | GYVRC | QTSNVD | C99C99.R99R99. | G99G99.Y99Y99. | Q99Q99.T99T99. | 21 | 15 | 18 | 54 | Yes | Yes | Yes |
| | | | | | A99A99.N99N99. | V99V99.R99R99. | S99S99.N99N99. | | | | | | | |
| | | | | | E99E99.V99V99. | C99C99. | V99V99.D99D99. | | | | | | | |
| | | | | | V99V99. | | | | | | | | | |
| 692 | 6 | PHCSDRL | HRYVQ | LLWSNDSFPR | P99P99.H99H99. | H99H99.R99R99. | L99L99.L99L99. | 21 | 15 | 30 | 66 | Yes | Yes | Yes |
| | | | | | C99C99.S99S99. | Y99Y99.V99V99. | W99W99.S99S99. | | | | | | | |
| | | | | | D99D99.R99R99. | Q99Q99. | N99N99.D99D99. | | | | | | | |
| | | | | | L99L99. | | S99S99.F99F99. | | | | | | | |
| | | | | | | | P99P99.R99R99. | | | | | | | |
| 694 | 6 | YVSYVIY | NGEDP | HRYSANGSYP | Y99Y99.V99V99. | N99N99.G99G99. | H66B00.R66B00. | 21 | 15 | 20 | 56 | Yes | Yes | Yes |
| | | | | | S99S99.Y99Y99. | E99E99.D99D99. | Y66B00.S66B00. | | | | | | | |
| | | | | | V99V99.I83B00. | P99P99. | A66B00.N66B00. | | | | | | | |
| | | | | | Y99Y99. | | G66B00.S66B00. | | | | | | | |
| | | | | | | | Y66B00.P66B00. | | | | | | | |
| 709 | 6 | HLYMILI | SRLEH | DIVLPSGQVG | H99H99.L99L99. | S99S99.R99R99. | D99D99.I99I99. | 21 | 15 | 30 | 66 | Yes | Yes | Yes |
| | | | | | Y99Y99.M99M99. | L99L99.E99E99. | V99V99.L99L99. | | | | | | | |
| | | | | | I99I99.L99L99. | H99H99. | P99P99.S99S99. | | | | | | | |
| | | | | | I99I99. | | G99G99.Q99Q99. | | | | | | | |
| | | | | | | | V99V99.G99G99. | | | | | | | |
| 716 | 6 | YLAVLAW | SVNG | CGEY | Y99Y99.L99L99. | S99S99.V99V99. | C99C99.G99G99. | 21 | 12 | 12 | 45 | Yes | Yes | Yes |
| | | | | | A99A99.V99V99. | N99N99.G99G99. | E99E99.Y99Y99. | | | | | | | |
| | | | | | L99L99.A99A99. | | | | | | | | | |
| | | | | | W99W99. | | | | | | | | | |
| 723 | 6 | YIERLWV | PPLPR | LSTCVHGYRP | Y99Y99.I99I99. | P99P99.P99P99. | L99L99.S99S99. | 21 | 15 | 30 | 66 | Yes | Yes | Yes |
| | | | | | E99E99.R99R99. | L99L99.P99P99. | T99T99.C99C99. | | | | | | | |
| | | | | | L99L99.W99W99. | R99R99. | V99V99.H99H99. | | | | | | | |
| | | | | | V99V99. | | G99G99.Y99Y99. | | | | | | | |
| | | | | | | | R99R99.P99P99. | | | | | | | |
| 744 | 6 | DGTRCVF | RVGLT | AYFDNHSRVD | D99D99.G99G99. | R99R99.V99V99. | A99A99.Y99Y99. | 21 | 15 | 30 | 66 | Yes | Yes | Yes |
| | | | | | T83B00.R99R99. | G99G99.L99L99. | F99F99.D99D99. | | | | | | | |
| | | | | | C99C99.V99V99. | T99T99. | N99N99.H99H99. | | | | | | | |
| | | | | | F99F99. | | S99S99.R99R99. | | | | | | | |
| | | | | | | | V99V99.D99D99. | | | | | | | |
| 758 | 6 | GTYCVHL | PDMSV | LLTTTYGQLF | G99G99.T99T99. | P99P99.D99D99. | L99L99.L99L99. | 21 | 15 | 30 | 66 | Yes | Yes | Yes |
| | | | | | Y99Y99.C99C99. | M99M99.S99S99. | T99T99.T99T99. | | | | | | | |
| | | | | | V99V99.H99H99. | V99V99. | T99T99.Y99Y99. | | | | | | | |
| | | | | | L99L99. | | G99G99.Q99Q99. | | | | | | | |
| | | | | | | | L99L99.F99F99. | | | | | | | |
| 765 | 6 | GCIQSVV | GIIGV | YRRLPHVGVW | G99G99.C99C99. | G99G99.I99I99. | Y99Y99.R99R99. | 21 | 15 | 30 | 66 | Yes | Yes | Yes |
| | | | | | I99I99.Q99Q99. | I99I99.G99G99. | R99R99.L99L99. | | | | | | | |
| | | | | | S99S99.V99V99. | V99V99. | P99P99.H99H99. | | | | | | | |
| | | | | | V99V99. | | V99V99.G99G99. | | | | | | | |
| | | | | | | | V99V99.W99W99. | | | | | | | |
| 772 | 6 | DALQ | CFEYY | FNWQPRGRV | D99D99.A99A99. | C99C99.F99F99. | F99F99.N99N99. | 12 | 15 | 27 | 54 | Yes | Yes | Yes |
| | | | | | L99L99.Q99Q99. | E99E99.Y99Y99. | W99W99.Q99Q99. | | | | | | | |
| | | | | | | Y99Y99. | P99P99.R99R99. | | | | | | | |
| | | | | | | | G99G99.R99R99. | | | | | | | |
| | | | | | | | V99V99. | | | | | | | |
| 776 | 6 | CWCNEAH | GCSFV | FD | C99C99.W99W99. | G99G99.C99C99. | F99F99.D99D99. | 21 | 15 | 6 | 42 | Yes | Yes | Yes |
| | | | | | C99C99.N99N99. | S99S99.F99F99. | | | | | | | | |
| | | | | | E99E99.A99A99. | V99V99. | | | | | | | | |
| | | | | | H99H99. | | | | | | | | | |
| 790 | 6 | MMCGIVQ | PCE | HALCLTCTQGVIL | M99M99.M99M99. | P99P99.C99C99. | H99H99.A99A99. | 21 | 9 | 39 | 69 | Yes | Yes | Yes |
| | | | | | C99C99.G99G99. | E99E99. | L99L99.C99C99. | | | | | | | |
| | | | | | I99I99.V99V99. | | L99L99.T99T99. | | | | | | | |
| | | | | | Q99Q99. | | C99C99.T99T99. | | | | | | | |
| | | | | | | | Q99Q99.G99G99. | | | | | | | |
| | | | | | | | V99V99.I99I99. | | | | | | | |
| | | | | | | | L99L99. | | | | | | | |
| 793 | 6 | NYVAYLL | STMAS | VDLCLL | N99N99.Y99Y99. | S99S99.T99T99. | V99V99.D99D99. | 21 | 15 | 18 | 54 | Yes | Yes | Yes |
| | | | | | V99V99.A99A99. | M99M99.A99A99. | L99L99.C99C99. | | | | | | | |
| | | | | | Y99Y99.L99L99. | S99S99. | L99L99.L99L99. | | | | | | | |
| | | | | | L99L99. | | | | | | | | | |
| 803 | 6 | R | PAIMC | LQVGFG | R99R99. | P99P99.A99A99. | L99L99.Q99Q99. | 3 | 15 | 18 | 36 | Yes | Yes | Yes |
| | | | | | | I99I99.M99M99. | V99V99.G99G99. | | | | | | | |
| | | | | | | C99C99. | F99F99.G99G99. | | | | | | | |
| 811 | 6 | LTHSCVY | GCGPY | ALYISSNHVH | L99L99.T99T99. | G99G99.C99C99. | A99A99.L99L99. | 21 | 15 | 30 | 66 | Yes | Yes | Yes |
| | | | | | H99H99.S99S99. | G99G99.P99P99. | Y99Y99.I99I99. | | | | | | | |
| | | | | | C99C99.V99V99. | Y99Y99. | S99S99.S99S99. | | | | | | | |
| | | | | | Y99Y99. | | N99N99.H99H99. | | | | | | | |
| | | | | | | | V99V99.H99H99. | | | | | | | |
| 813 | 6 | AVNAQVW | RGSHC | AYWAPGGVF | A99A99.V99V99. | R99R99.G99G99. | A99A99.Y99Y99. | 21 | 15 | 27 | 63 | Yes | Yes | Yes |
| | | | | | N99N99.A99A99. | S99S99.H99H99. | W99W99.A99A99. | | | | | | | |
| | | | | | Q99Q99.V99V99. | C99C99. | P99P99.G99G99. | | | | | | | |
| | | | | | W99W99. | | G99G99.V99V99. | | | | | | | |
| | | | | | | | F99F99. | | | | | | | |
| 815 | 6 | NHRPIVS | GAIAV | NSYRGVGLIL | N99N99.H99H99. | G99G99.A99A99. | N99N99.S99S99. | 21 | 15 | 30 | 66 | Yes | Yes | Yes |
| | | | | | R99R99.P99P99. | I99I99.A99A99. | Y99Y99.R99R99. | | | | | | | |
| | | | | | I99I99.V99V99. | V99V99. | G99G99.V99V99. | | | | | | | |
| | | | | | S99S99. | | G99G99.L99L99. | | | | | | | |
| | | | | | | | I99I99.L99L99. | | | | | | | |
| 822 | 6 | HPYEDSA | RFGDV | IHLVRNKLLA | H99H99.P99P99. | R99R99.F99F99. | I99I99.H99H99. | 21 | 15 | 30 | 66 | Yes | Yes | Yes |
| | | | | | Y99Y99.E99E99. | G99G99.D99D99. | L99L99.V99V99. | | | | | | | |
| | | | | | D99D99.S99S99. | V99V99. | R99R99.N99N99. | | | | | | | |
| | | | | | A99A99. | | K99K99.L99L99. | | | | | | | |
| | | | | | | | L99L99.A99A99. | | | | | | | |
| 824 | 6 | WR | PSFGY | AHVGLNCCVI | W99W99.R99R99. | P99P99.S99S99. | A99A99.H99H99. | 6 | 15 | 30 | 51 | Yes | Yes | Yes |
| | | | | | | F99F99.G99G99. | V99V99.G99G99. | | | | | | | |
| | | | | | | Y99Y99. | L99L99.N99N99. | | | | | | | |
| | | | | | | | C83B00.C99C99. | | | | | | | |
| | | | | | | | V99V99.I99I99. | | | | | | | |
| 831 | 6 | EWRSVVW | PGATG | TIFSHGP | E99E99.W99W99. | P99P99.G99G99. | T99T99.I99I99. | 21 | 15 | 30 | 66 | Yes | Yes | Yes |
| | | | | | R99R99.S99S99. | A99A99.T99T99. | F99F99.S99S99. | | | | | | | |
| | | | | | V99V99.V99V99. | G99G99. | H99H99.G99G99. | | | | | | | |
| | | | | | W99W99. | | P99P99.V99V99. | | | | | | | |
| | | | | | | | L99L99.T99T99. | | | | | | | |
| 833 | 6 | LLRKCVF | NGICV | VIYSPAAVE | L99L99.L99L99. | N99N99.G99G99. | V99V99.I99I99. | 21 | 15 | 27 | 63 | Yes | Yes | Yes |
| | | | | | R99R99.K99K99. | I99I99.C99C99. | Y99Y99.S99S99. | | | | | | | |
| | | | | | C99C99.V99V99. | V99V99. | P99P99.A99A99. | | | | | | | |
| | | | | | F99F99. | | A99A99.V99V99. | | | | | | | |
| | | | | | | | E99E99. | | | | | | | |
| 843 | 6 | VGLLCVV | PSTF | FQPSACCSVR | V99V99.G99G99. | P99P99.S83B00. | F99F99.Q99Q99. | 21 | 12 | 30 | 63 | Yes | Yes | Yes |
| | | | | | L99L99.L99L99. | T99T99.F99F99. | P99P99.S99S99. | | | | | | | |
| | | | | | C99C99.V99V99. | | A99A99.C99C99. | | | | | | | |
| | | | | | V99V99. | | C99C99.S99S99. | | | | | | | |
| | | | | | | | V99V99.R99R99. | | | | | | | |
| 845 | 6 | ILSCWYV | IAPVN | FPAYSACFHF | I99I99.L99L99. | I99I99.A99A99. | F99F99.P99P99. | 21 | 15 | 30 | 66 | Yes | Yes | Yes |
| | | | | | S99S99.C99C99. | P99P99.V99V99. | A99A99.Y99Y99. | | | | | | | |
| | | | | | W99W99.Y99Y99. | N99N99. | S99S99.A99A99. | | | | | | | |
| | | | | | V99V99. | | C99C99.F99F99. | | | | | | | |
| | | | | | | | H99H99.F99F99. | | | | | | | |
| 848 | 6 | MSLSSST | MFLFV | FLTNVL | M99M99.S99S99. | M99M99.F99F99. | F99F99.L99L99. | 21 | 15 | 18 | 54 | Yes | Yes | Yes |
| | | | | | L99L99.S99S99. | L99L99.F99F99. | T99T99.N99N99. | | | | | | | |
| | | | | | S99S99.S99S99. | V99V99. | V99V99.L99L99. | | | | | | | |
| | | | | | T99T99. | | | | | | | | | |
| 850 | 6 | MVNANVY | RDHYV | AYCCSASGRYTVA | M99M99.V99V99. | R99R99.D99D99. | A99A99.Y99Y99. | 21 | 15 | 39 | 75 | Yes | Yes | Yes |
| | | | | | N99N99.A99A99. | H99H99.Y99Y99. | C99C99.C99C99. | | | | | | | |
| | | | | | N99N99.V99V99. | V99V99. | S99S99.A99A99. | | | | | | | |
| | | | | | Y99Y99. | | S99S99.G99G99. | | | | | | | |
| | | | | | | | R83B00.Y99Y99. | | | | | | | |
| | | | | | | | T99T99.V99V99. | | | | | | | |
| | | | | | | | A99A99. | | | | | | | |
| 851 | 6 | ACVV | ATHLN | YCFGDS | A99A99.C99C99. | A99A99.T99T99. | B00 | 12 | 15 | 0 | 27 | Yes | Yes | Yes |
| | | | | | V99V99.V99V99. | H99H99.L99L99. | | | | | | | | |
| | | | | | | N99N99. | | | | | | | | |
| 857 | 6 | WNGTALN | PVFAT | FCYNTSCVIE | W99W99.N83B00. | P99P99.V99V99. | F99F99.C99C99. | 21 | 15 | 30 | 66 | Yes | Yes | Yes |
| | | | | | G99G99.T99T99. | F99F99.A99A99. | Y99Y99.N99N99. | | | | | | | |
| | | | | | A99A99.L99L99. | T99T99. | T99T99.S99S99. | | | | | | | |
| | | | | | N99N99. | | C99C99.V99V99. | | | | | | | |
| | | | | | | | I99I99.E99E99. | | | | | | | |
| 862 | 6 | WVVRECF | VDASS | AALVTLVSVV | W99W99.V99V99. | V99V99.D99D99. | B00 | 21 | 15 | 0 | 36 | Yes | Yes | Yes |
| | | | | | V99V99.R83B00. | A99A99.S99S99. | | | | | | | | |
| | | | | | E99E99.C99C99. | S99S99. | | | | | | | | |
| | | | | | F99F99. | | | | | | | | | |
| 865 | 6 | PTIGCCY | VGSIS | LYATGSSEELTIH | P99P99.T99T99. | V99V99.G99G99. | L99L99.Y99Y99. | 21 | 15 | 39 | 75 | Yes | Yes | Yes |
| | | | | | I99I99.G99G99. | S99S99.I99I99. | A99A99.T99T99. | | | | | | | |
| | | | | | C99C99.C99C99. | S99S99. | G99G99.S99S99. | | | | | | | |
| | | | | | Y99Y99. | | S99S99.E99E99. | | | | | | | |
| | | | | | | | E99E99.L99L99. | | | | | | | |
| | | | | | | | T83B00.I99I99. | | | | | | | |
| | | | | | | | H99H99. | | | | | | | |
| 870 | 6 | YLSRRIF | VLGVP | YCIDCLFTVV | Y99Y99.L99L99. | V99V99.L99L99. | B00 | 21 | 15 | 0 | 36 | Yes | Yes | Yes |
| | | | | | S99S99.R99R99. | G99G99.V99V99. | | | | | | | | |
| | | | | | R99R99.I99I99. | P99P99. | | | | | | | | |
| | | | | | F99F99. | | | | | | | | | |
| 872 | 6 | DMLVIYG | TCSFE | NIHVTGYGNC | D99D99.M99M99. | T99T99.C99C99. | N99N99.I99I99. | 21 | 15 | 30 | 66 | Yes | Yes | Yes |
| | | | | | L99L99.V99V99. | S99S99.F99F99. | H99H99.V99V99. | | | | | | | |
| | | | | | I99I99.Y83B00. | E99E99. | T99T99.G99G99. | | | | | | | |
| | | | | | G99G99. | | Y99Y99.G99G99. | | | | | | | |
| | | | | | | | N99N99.C99C99. | | | | | | | |
| 873 | 6 | HIAGCHA | YGNMI | PGF | H99H99.I99I99. | Y99Y99.G99G99. | P99P99.G99G99. | 21 | 15 | 9 | 45 | Yes | Yes | Yes |
| | | | | | A99A99.G99G99. | N99N99.M99M99. | F99F99. | | | | | | | |
| | | | | | C99C99.H99H99. | I99I99. | | | | | | | | |
| | | | | | A99A99. | | | | | | | | | |
| 881 | 6 | TLGRNVY | VLD | VYLSRLSNVL | T99T99.L99L99. | V99V99.L99L99. | V99V99.Y99Y99. | 21 | 9 | 30 | 60 | Yes | Yes | Yes |
| | | | | | G99G99.R99R99. | D99D99. | L99L99.S99S99. | | | | | | | |
| | | | | | N99N99.V99V99. | | R99R99.L99L99. | | | | | | | |
| | | | | | Y99Y99. | | S99S99.N99N99. | | | | | | | |
| | | | | | | | V99V99.L99L99. | | | | | | | |
| 886 | 6 | LCFHSYC | FARKG | NDIYWV | L99L99.C99C99. | F99F99.A99A99. | N99N99.D99D99. | 21 | 15 | 18 | 54 | Yes | Yes | Yes |
| | | | | | F99F99.H99H99. | R99R99.K99K99. | I99I99.Y99Y99. | | | | | | | |
| | | | | | S99S99.Y83B00. | G99G99. | W99W99.V99V99. | | | | | | | |
| | | | | | C99C99. | | | | | | | | | |
| 892 | 6 | DGWLVGC | SWQSS | NSWNVCFGD | D99D99.G99G99. | S99S99.W99W99. | N99N99.S99S99. | 21 | 15 | 27 | 63 | Yes | Yes | Yes |
| | | | | | W99W99.L99L99. | Q99Q99.S99S99. | W99W99.F99F99. | | | | | | | |
| | | | | | V99V99.G99G99. | S99S99. | G99G99.D99D99. | | | | | | | |
| | | | | | C99C99. | | N99N99.V99V99. | | | | | | | |
| | | | | | | | C99C99. | | | | | | | |
| 898 | 6 | GPYCWER | MCFND | SPSGYCLFLL | G99G99.P99P99. | M99M99.C99C99. | S99S99.P99P99. | 21 | 15 | 30 | 66 | Yes | Yes | Yes |
| | | | | | Y99Y99.C99C99. | F99F99.N99N99. | S99S99.G99G99. | | | | | | | |
| | | | | | W99W99.E99E99. | D99D99. | Y99Y99.C99C99. | | | | | | | |
| | | | | | R99R99. | | L99L99.F99F99. | | | | | | | |
| | | | | | | | L99L99.L99L99. | | | | | | | |
| 908 | 6 | VLEFLVY | TGGCC | ACYNTTAICA | V99V99.L99L99. | T99T99.G99G99. | B00 | 21 | 15 | 0 | 36 | Yes | Yes | Yes |
| | | | | | E99E99.F99F99. | G99G99.C99C99. | | | | | | | | |
| | | | | | L99L99.V99V99. | C99C99. | | | | | | | | |
| | | | | | Y99Y99. | | | | | | | | | |
| 913 | 6 | VVTRKVF | PDLAS | YCNSRTGVVV | V99V99.V83B00. | P99P99.D99D99. | B00 | 21 | 15 | 0 | 36 | Yes | Yes | Yes |
| | | | | | T99T99.R99R99. | L99L99.A99A99. | | | | | | | | |
| | | | | | K99K99.V99V99. | S99S99. | | | | | | | | |
| | | | | | F99F99. | | | | | | | | | |
| 914 | 6 | TYDHCFG | EMEWL | NMWISSALG | T99T99.Y83B00. | E99E99.M99M99. | N99N99.M99M99. | 21 | 15 | 27 | 63 | Yes | Yes | Yes |
| | | | | | D99D99.H99H99. | E99E99.W99W99. | W99W99.I99I99. | | | | | | | |
| | | | | | C99C99.F99F99. | L99L99. | S99S99.S99S99. | | | | | | | |
| | | | | | G99G99. | | A99A99.L99L99. | | | | | | | |
| | | | | | | | G99G99. | | | | | | | |
| 916 | 6 | DWTRIVF | TAGGH | FRLSLSGSII | D99D99.W99W99. | T99T99.A99A99. | F99F99.R99R99. | 21 | 15 | 30 | 66 | Yes | Yes | Yes |
| | | | | | T99T99.R83B00. | G99G99.G99G99. | L99L99.S99S99. | | | | | | | |
| | | | | | I99I99.V99V99. | H99H99. | L99L99.S99S99. | | | | | | | |
| | | | | | F99F99. | | G99G99.S99S99. | | | | | | | |
| | | | | | | | I99I99.I99I99. | | | | | | | |
| 103 | 5 | TYSALVW | ELGCD | VMLLYSRDHCCAQ | T99T99.Y99Y99. | E99E99.L99L99. | V99V99.M99M99. | 21 | 15 | 39 | 75 | Yes | Yes | Yes |
| | | | | | S99S99.A99A99. | G99G99.C99C99. | L99L99.L99L99. | | | | | | | |
| | | | | | L99L99.V99V99. | D99D99. | Y99Y99.S99S99. | | | | | | | |
| | | | | | W99W99. | | R99R99.D99D99. | | | | | | | |
| | | | | | | | H99H99.C99C99. | | | | | | | |
| | | | | | | | C99C99.A99A99. | | | | | | | |
| | | | | | | | Q99Q99. | | | | | | | |
| 12 | 5 | LNVFA | TCGPR | HHYEHNYMLN | L99L99.N99N99. | T99T99.C99C99. | H99H99.H99H99. | 21 | 15 | 30 | 66 | Yes | Yes | Yes |
| | | | | | R99R99.D99D99. | G99G99.P99P99. | Y99Y99.E99E99. | | | | | | | |
| | | | | | V99V99.F99F99. | R99R99. | H99H99.N99N99. | | | | | | | |
| | | | | | A99A99. | | Y99Y99.M99M99. | | | | | | | |
| | | | | | | | L99L99.N99N99. | | | | | | | |
| 126 | 5 | CSNEYPL | ALHKV | VSPSLS | C99C99.S99S99. | A99A99.L99L99. | V99V99.S99S99. | 21 | 15 | 18 | 54 | Yes | Yes | Yes |
| | | | | | N99N99.E99E99. | H99H99.K99K99. | P99P99.S99S99. | | | | | | | |
| | | | | | Y99Y99.P99P99. | V99V99. | L99L99.S99S99. | | | | | | | |
| | | | | | L99L99. | | | | | | | | | |
| 173 | 5 | CPNTRVY | SSGAL | YYYCALNCLD | C99C99.P99P99. | S99S99.S99S99. | Y99Y99.Y99Y99. | 21 | 15 | 30 | 66 | Yes | Yes | Yes |
| | | | | | N99N99.T99T99. | G99G99.A99A99. | Y99Y99.C99C99. | | | | | | | |
| | | | | | R99R99.V99V99. | L99L99. | A99A99.L99L99. | | | | | | | |
| | | | | | Y99Y99. | | N99N99.C99C99. | | | | | | | |
| | | | | | | | L99L99.D99D99. | | | | | | | |
| 183 | 5 | GGCSCST | LV | IAHRCYCCF | G99G99.G99G99. | L99L99.V99V99. | I99I99.A99A99. | 21 | 6 | 27 | 54 | Yes | Yes | Yes |
| | | | | | C99C99.S99S99. | | H99H99.R99R99. | | | | | | | |
| | | | | | C99C99.S99S99. | | C99C99.Y99Y99. | | | | | | | |
| | | | | | T99T99. | | C99C99.C99C99. | | | | | | | |
| | | | | | | | F99F99. | | | | | | | |
| 197 | 5 | VTVLSLG | GLKRA | HFFARHFVD | V99V99.T99T99. | G99G99.L99L99. | H99H99.F99F99. | 16 | 15 | 27 | 58 | Yes | Yes | Yes |
| | | | | | V80B00.L80B00. | K99K99.R99R99. | F99F99.A99A99. | | | | | | | |
| | | | | | S80B00.L80B00. | A99A99. | R99R99.H99H99. | | | | | | | |
| | | | | | G80B00. | | F99F99.V99V99. | | | | | | | |
| | | | | | | | D99D99. | | | | | | | |
| 198 | 5 | VVCNRIF | NDMCG | AVLTVCGLVT | V99V99.V99V99. | N99N99.D99D99. | A99A99.V99V99. | 21 | 15 | 30 | 66 | Yes | Yes | Yes |
| | | | | | C99C99.N99N99. | M99M99.C99C99. | L99L99.T99T99. | | | | | | | |
| | | | | | R99R99.I99I99. | G99G99. | V99V99.C99C99. | | | | | | | |
| | | | | | F99F99. | | G99G99.L99L99. | | | | | | | |
| | | | | | | | V99V99.T99T99. | | | | | | | |
| 215 | 5 | VRSAWFY | PHYRG | PLFYNVLTGSLSG | V99V99.R99R99. | P99P99.H99H99. | P99P99.L99L99. | 21 | 15 | 39 | 75 | Yes | Yes | Yes |
| | | | | | S99S99.A99A99. | Y99Y99.R99R99. | F99F99.Y99Y99. | | | | | | | |
| | | | | | W99W99.F99F99. | G99G99. | N99N99.V99V99. | | | | | | | |
| | | | | | Y99Y99. | | L99L99.T99T99. | | | | | | | |
| | | | | | | | G99G99.S99S99. | | | | | | | |
| | | | | | | | L99L99.S99S99. | | | | | | | |
| | | | | | | | G99G99. | | | | | | | |
| 23 | 5 | WYVDTSH | AVSDH | VCQARW | W99W99.Y99Y99. | A99A99.V99V99. | V99V99.C99C99. | 20 | 15 | 18 | 53 | Yes | Yes | Yes |
| | | | | | V99V99.D80B00. | S99S99.D99D99. | Q99Q99.A99A99. | | | | | | | |
| | | | | | T99T99.S99S99. | H99H99. | R99R99.W99W99. | | | | | | | |
| | | | | | H99H99. | | | | | | | | | |
| 233 | 5 | VHFKYSC | CLLDR | FLIDRRDDFVLCM | V99V99.H80B00. | C99C99.L99L99. | F99F99.L99L99. | 20 | 15 | 39 | 74 | Yes | Yes | Yes |
| | | | | | F99F99.K99K99. | L99L99.D99D99. | I99I99.D99D99. | | | | | | | |
| | | | | | Y99Y99.S99S99. | R99R99. | R99R99.R99R99. | | | | | | | |
| | | | | | C99C99. | | D99D99.D99D99. | | | | | | | |
| | | | | | | | F99F99.V99V99. | | | | | | | |
| | | | | | | | L99L99.C99C99. | | | | | | | |
| | | | | | | | M99M99. | | | | | | | |
| 246 | 5 | CLSHTVY | AGSLG | TLIDSQGYVA | C99C99.L99L99. | A99A99.G99G99. | T99T99.L99L99. | 21 | 15 | 30 | 66 | Yes | Yes | Yes |
| | | | | | S99S99.H99H99. | S99S99.L99L99. | I99I99.D99D99. | | | | | | | |
| | | | | | T99T99.V99V99. | G99G99. | S99S99.Q99Q99. | | | | | | | |
| | | | | | Y99Y99. | | G99G99.Y99Y99. | | | | | | | |
| | | | | | | | V99V99.A99A99. | | | | | | | |
| 262 | 5 | LRSLLG | RSDTD | HVYSLTGAVM | L99L99.R99R99. | R99R99.S99S99. | H99H99.V99V99. | 18 | 15 | 30 | 63 | Yes | Yes | Yes |
| | | | | | S99S99.L99L99. | D99D99.T99T99. | Y99Y99.S99S99. | | | | | | | |
| | | | | | L99L99.G99G99. | D99D99. | L99L99.T99T99. | | | | | | | |
| | | | | | | | G99G99.A99A99. | | | | | | | |
| | | | | | | | V99V99.M99M99. | | | | | | | |
| 279 | 5 | VRVLGVV | TRACN | TYFAHSSVLT | V99V99.R99R99. | T99T99.R99R99. | T80B00.Y99Y99. | 21 | 15 | 22 | 58 | Yes | Yes | Yes |
| | | | | | V99V99.L99L99. | A99A99.C99C99. | F80B00.A80B00. | | | | | | | |
| | | | | | G99G99.V99V99. | N99N99. | H80B00.S80B00. | | | | | | | |
| | | | | | V99V99. | | S99S99.V80B00. | | | | | | | |
| | | | | | | | L80B00.T80B00. | | | | | | | |
| 283 | 5 | LPLPPIR | ILTRW | LYNLSTSVME | L99L99.P99P99. | I99I99.L99L99. | L99L99.Y99Y99. | 21 | 15 | 30 | 66 | Yes | Yes | Yes |
| | | | | | L99L99.P99P99. | T99T99.R99R99. | N99N99.L99L99. | | | | | | | |
| | | | | | P99P99.I99I99. | W99W99. | S99S99.T99T99. | | | | | | | |
| | | | | | R99R99. | | S99S99.V99V99. | | | | | | | |
| | | | | | | | M99M99.E99E99. | | | | | | | |
| 291 | 5 | LWASEFG | EEDGI | MICSYR | L99L99.W99W99. | E99E99.E99E99. | M99M99.I99I99. | 21 | 15 | 18 | 54 | Yes | Yes | Yes |
| | | | | | A99A99.S99S99. | D99D99.G99G99. | C99C99.S99S99. | | | | | | | |
| | | | | | E99E99.F99F99. | I99I99. | Y99Y99.R99R99. | | | | | | | |
| | | | | | G99G99. | | | | | | | | | |
| 294 | 5 | WSNCFIV | SVTHA | LDTGLC | W99W99.S99S99. | S99S99.V99V99. | L99L99.D99D99. | 21 | 15 | 18 | 54 | Yes | Yes | Yes |
| | | | | | N99N99.C99C99. | T99T99.H99H99. | T99T99.G99G99. | | | | | | | |
| | | | | | F99F99.I99I99. | A99A99. | L99L99.C99C99. | | | | | | | |
| | | | | | V99V99. | | | | | | | | | |
| 309 | 5 | NLFRWAH | DWGQS | PIVTGHGTVV | N99N99.L99L99. | D99D99.W99W99. | P99P99.I99I99. | 21 | 15 | 30 | 66 | Yes | Yes | Yes |
| | | | | | F99F99.R99R99. | G99G99.Q99Q99. | V99V99.T99T99. | | | | | | | |
| | | | | | W99W99.A99A99. | S99S99. | G99G99.H99H99. | | | | | | | |
| | | | | | H99H99. | | G99G99.T99T99. | | | | | | | |
| | | | | | | | V99V99.V99V99. | | | | | | | |
| 331 | 5 | LSCFCRV | SRACT | FPSIHSEDCY | L99L99.S99S99. | S99S99.R99R99. | F99F99.P99P99. | 21 | 15 | 30 | 66 | Yes | Yes | Yes |
| | | | | | C99C99.F99F99. | A99A99.C99C99. | S99S99.I99I99. | | | | | | | |
| | | | | | C99C99.R99R99. | T99T99. | H99H99.S99S99. | | | | | | | |
| | | | | | V99V99. | | E99E99.D99D99. | | | | | | | |
| | | | | | | | C99C99.Y99Y99. | | | | | | | |
| 363 | 5 | YGCGLVY | KQSLG | WFFRNDLDPQ | Y99Y99.G99G99. | K99K99.Q99Q99. | W99W99.F99F99. | 21 | 15 | 30 | 66 | Yes | Yes | Yes |
| | | | | | C99C99.G99G99. | S99S99.L99L99. | F99F99.R99R99. | | | | | | | |
| | | | | | L99L99.V99V99. | G99G99. | N99N99.D99D99. | | | | | | | |
| | | | | | Y99Y99. | | L99L99.D99D99. | | | | | | | |
| | | | | | | | P99P99.Q99Q99. | | | | | | | |
| 380 | 5 | RNFNTGD | RSFLV | FAVCTGLGVF | R99R99.N99N99. | R99R99.S99S99. | F99F99.A99A99. | 21 | 15 | 30 | 66 | Yes | Yes | Yes |
| | | | | | F99F99.N99N99. | F99F99.L99L99. | V99V99.C99C99. | | | | | | | |
| | | | | | T99T99.G99G99. | V99V99. | T99T99.G99G99. | | | | | | | |
| | | | | | D99D99. | | L99L99.G99G99. | | | | | | | |
| | | | | | | | V99V99.F99F99. | | | | | | | |
| 389 | 5 | YDILYMF | PGRYV | SPDFSL | Y99Y99.D99D99. | P99P99.G99G99. | S99S99.P99P99. | 21 | 15 | 18 | 54 | Yes | Yes | Yes |
| | | | | | I99I99.L99L99. | R99R99.Y99Y99. | D99D99.F99F99. | | | | | | | |
| | | | | | Y99Y99.M99M99. | V99V99. | S99S99.L99L99. | | | | | | | |
| | | | | | F99F99. | | | | | | | | | |
| 404 | 5 | YCYHTYY | HDDNM | GVYVQWRGVY | Y99Y99.C99C99. | H99H99.D99D99. | G99G99.V99V99. | 21 | 15 | 30 | 66 | Yes | Yes | Yes |
| | | | | | Y99Y99.H99H99. | D99D99.N99N99. | Y99Y99.V99V99. | | | | | | | |
| | | | | | T99T99.Y99Y99. | M99M99. | Q99Q99.W99W99. | | | | | | | |
| | | | | | Y99Y99. | | R99R99.G99G99. | | | | | | | |
| | | | | | | | V99V99.Y99Y99. | | | | | | | |
| 407 | 5 | LIS | RTCSS | CCLREDLTCF | G99G99.Y99Y99. | R99R99.T99T99. | C99C99.C99C99. | 21 | 15 | 30 | 66 | Yes | Yes | Yes |
| | | | | | V99V99.V99V99. | C99C99.S99S99. | L99L99.R99R99. | | | | | | | |
| | | | | | L99L99.I99I99. | S99S99. | E99E99.D99D99. | | | | | | | |
| | | | | | S99S99. | | L99L99.T99T99. | | | | | | | |
| | | | | | | | C99C99.F99F99. | | | | | | | |
| 411 | 5 | SWPAFAC | GCNRN | VDQSW | S80B00.W99W99. | G99G99.C99C99. | V99V99.D99D99. | 20 | 15 | 15 | 50 | Yes | Yes | Yes |
| | | | | | P99P99.A99A99. | N99N99.R99R99. | Q99Q99.S99S99. | | | | | | | |
| | | | | | F99F99.A99A99. | N99N99. | W99W99. | | | | | | | |
| | | | | | C99C99. | | | | | | | | | |
| 429 | 5 | FIQQLVF | IDGVS | YTLCAG | F99F99.I99I99. | I99I99.D99D99. | Y99Y99.T99T99. | 21 | 15 | 18 | 54 | Yes | Yes | Yes |
| | | | | | Q99Q99.Q99Q99. | G99G99.V99V99. | L99L99.C99C99. | | | | | | | |
| | | | | | L99L99.V99V99. | S99S99. | A99A99.G99G99. | | | | | | | |
| | | | | | F99F99. | | | | | | | | | |
| 435 | 5 | SVIYVMW | KLTES | AVNCCL | S99S99.V99V99. | K99K99.L99L99. | A99A99.V99V99. | 21 | 15 | 18 | 54 | Yes | Yes | Yes |
| | | | | | I99I99.Y99Y99. | T99T99.E99E99. | N99N99.C99C99. | | | | | | | |
| | | | | | V99V99.M99M99. | S99S99. | C99C99.L99L99. | | | | | | | |
| | | | | | W99W99. | | | | | | | | | |
| 446 | 5 | GISALLF | CPERR | IDMYGC | G99G99.I99I99. | C99C99.P99P99. | I99I99.D99D99. | 21 | 15 | 18 | 54 | Yes | Yes | Yes |
| | | | | | S99S99.A99A99. | E99E99.R99R99. | M99M99.Y99Y99. | | | | | | | |
| | | | | | L99L99.L99L99. | R99R99. | G99G99.C99C99. | | | | | | | |
| | | | | | F99F99. | | | | | | | | | |
| 453 | 5 | NIICVFS | LRCAG | LGTLTPVSCD | N99N99.I99I99. | L99L99.R99R99. | L99L99.G99G99. | 21 | 15 | 30 | 66 | Yes | Yes | Yes |
| | | | | | I99I99.C99C99. | C99C99.A99A99. | T99T99.L99L99. | | | | | | | |
| | | | | | V99V99.F99F99. | G99G99. | T99T99.P99P99. | | | | | | | |
| | | | | | S99S99. | | V99V99.S99S99. | | | | | | | |
| | | | | | | | C99C99.D99D99. | | | | | | | |
| 459 | 5 | VYYVERA | GYGTV | PLC | V99V99.Y99Y99. | G99G99.Y99Y99. | P99P99.L99L99. | 21 | 15 | 9 | 45 | Yes | Yes | Yes |
| | | | | | Y99Y99.V99V99. | G99G99.T99T99. | C99C99. | | | | | | | |
| | | | | | E99E99.R99R99. | V99V99. | | | | | | | | |
| | | | | | A99A99. | | | | | | | | | |
| 465 | 5 | RFGSNLY | GVLVN | ICQWGC | R99R99.F99F99. | G99G99.V99V99. | I99I99.C99C99. | 21 | 15 | 18 | 54 | Yes | Yes | Yes |
| | | | | | G99G99.S99S99. | L99L99.V99V99. | Q99Q99.W99W99. | | | | | | | |
| | | | | | N99N99.L99L99. | N99N99. | G99G99.C99C99. | | | | | | | |
| | | | | | Y99Y99. | | | | | | | | | |
| 478 | 5 | YLVAYEN | MTCKS | PNGTVPHCSI | Y99Y99.L99L99. | M99M99.T99T99. | B00 | 21 | 15 | 0 | 36 | Yes | Yes | Yes |
| | | | | | V99V99.A99A99. | C99C99.K99K99. | | | | | | | | |
| | | | | | Y99Y99.E99E99. | S99S99. | | | | | | | | |
| | | | | | N99N99. | | | | | | | | | |
| 479 | 5 | NIDPV | VHPCN | FALLTTAGFF | N99N99.I99I99. | V99V99.H99H99. | F99F99.A99A99. | 21 | 15 | 30 | 66 | Yes | Yes | Yes |
| | | | | | F99F99.N99N99. | P99P99.C99C99. | L99L99.L99L99. | | | | | | | |
| | | | | | D99D99.P99P99. | N99N99. | T99T99.T99T99. | | | | | | | |
| | | | | | V99V99. | | A99A99.G99G99. | | | | | | | |
| | | | | | | | F99F99.F99F99. | | | | | | | |
| 487 | 5 | PSPVFMI | CCFFF | LHLDPNDRVL | P80B00.S99S99. | C99C99.C99C99. | L99L99.H99H99. | 20 | 15 | 30 | 65 | Yes | Yes | Yes |
| | | | | | P99P99.V99V99. | F99F99.F99F99. | L99L99.D99D99. | | | | | | | |
| | | | | | F99F99.M99M99. | F99F99. | P99P99.N99N99. | | | | | | | |
| | | | | | I99I99. | | D99D99.R99R99. | | | | | | | |
| | | | | | | | V99V99.L99L99. | | | | | | | |
| 492 | 5 | FSDFLQI | VGCGS | YQWRLSGRF | F99F99.S99S99. | V99V99.G99G99. | Y99Y99.Q99Q99. | 21 | 15 | 27 | 63 | Yes | Yes | Yes |
| | | | | | D99D99.P99P99. | C99C99.G99G99. | W99W99.R99R99. | | | | | | | |
| | | | | | L99L99.Q99Q99. | S99S99. | L99L99.S99S99. | | | | | | | |
| | | | | | I99I99. | | G99G99.R99R99. | | | | | | | |
| | | | | | | | F99F99. | | | | | | | |
| 505 | 5 | GFVGCID | NRLPA | WPIARQSDLRRF | G99G99.F99F99. | N99N99.R99R99. | W99W99.P99P99. | 21 | 15 | 36 | 72 | Yes | Yes | Yes |
| | | | | | V99V99.G99G99. | L99L99.P99P99. | I99I99.A99A99. | | | | | | | |
| | | | | | C99C99.I99I99. | A99A99. | R99R99.Q99Q99. | | | | | | | |
| | | | | | D99D99. | | S99S99.D99D99. | | | | | | | |
| | | | | | | | L99L99.R99R99. | | | | | | | |
| | | | | | | | R99R99.F99F99. | | | | | | | |
| 506 | 5 | VNTWRSL | PFDDD | SAYTCMGPQVVRL | V99V99.N99N99. | P99P99.F99F99. | S99S99.A99A99. | 21 | 15 | 38 | 74 | Yes | Yes | Yes |
| | | | | | T99T99.W99W99. | D99D99.D99D99. | Y99Y99.T99T99. | | | | | | | |
| | | | | | R99R99.S99S99. | D99D99. | C99C99.M99M99. | | | | | | | |
| | | | | | L99L99. | | G99G99.P99P99. | | | | | | | |
| | | | | | | | Q99Q99.V99V99. | | | | | | | |
| | | | | | | | V80B00.R99R99. | | | | | | | |
| | | | | | | | L99L99. | | | | | | | |
| 517 | 5 | RVFQV | CGCGV | SHFGIR | R99R99.V99V99. | C99C99.G99G99. | B00 | 14 | 15 | 0 | 29 | Yes | Yes | Yes |
| | | | | | F99F99.Q80B00. | C99C99.G99G99. | | | | | | | | |
| | | | | | V99V99. | V99V99. | | | | | | | | |
| 524 | 5 | SW | VFFCD | TGGTYH | S99S99.W99W99. | V99V99.F99F99. | T99T99.G99G99. | 6 | 15 | 18 | 39 | Yes | Yes | Yes |
| | | | | | | F99F99.C99C99. | G99G99.T99T99. | | | | | | | |
| | | | | | | D99D99. | Y99Y99.H99H99. | | | | | | | |
| 548 | 5 | YQLKFCG | MCGTL | CPRLDW | Y99Y99.Q80B00. | M99M99.C99C99. | C99C99.P99P99. | 20 | 15 | 18 | 53 | Yes | Yes | Yes |
| | | | | | L99L99.K99K99. | G99G99.T99T99. | R99R99.L99L99. | | | | | | | |
| | | | | | F99F99.C99C99. | L99L99. | D99D99.W99W99. | | | | | | | |
| | | | | | G99G99. | | | | | | | | | |
| 553 | 5 | AIRCCVY | SAGLA | VLYSPIGITK | A99A99.I99I99. | S99S99.A99A99. | V99V99.L99L99. | 21 | 15 | 30 | 66 | Yes | Yes | Yes |
| | | | | | R99R99.C99C99. | G99G99.L99L99. | Y99Y99.S99S99. | | | | | | | |
| | | | | | C99C99.V99V99. | A99A99. | P99P99.I99I99. | | | | | | | |
| | | | | | Y99Y99. | | G99G99.I99I99. | | | | | | | |
| | | | | | | | T99T99.K99K99. | | | | | | | |
| 567 | 5 | RSVVCSV | MRGCN | HCVLETYMT | R99R99.S99S99. | M99M99.R99R99. | B00 | 21 | 15 | 0 | 36 | Yes | Yes | Yes |
| | | | | | V99V99.V99V99. | G99G99.C99C99. | | | | | | | | |
| | | | | | C99C99.S99S99. | N99N99. | | | | | | | | |
| | | | | | V99V99. | | | | | | | | | |
| 578 | 5 | AAGARMS | NRATH | DPNPYIWPS | A99A99.A99A99. | N99N99.R99R99. | D99D99.P99P99. | 21 | 15 | 27 | 63 | Yes | Yes | Yes |
| | | | | | G99G99.A99A99. | A99A99.T99T99. | N99N99.P99P99. | | | | | | | |
| | | | | | R99R99.M99M99. | H99H99. | Y99Y99.I99I99. | | | | | | | |
| | | | | | S99S99. | | W99W99.P99P99. | | | | | | | |
| | | | | | | | S99S99. | | | | | | | |
| 579 | 5 | SYNRSTF | IDMVS | MICTPTLEVK | S99S99.Y99Y99. | I99I99.D99D99. | M99M99.I99I99. | 20 | 15 | 30 | 65 | Yes | Yes | Yes |
| | | | | | N99N99.R99R99. | M99M99.V99V99. | C99C99.T99T99. | | | | | | | |
| | | | | | S99S99.T80B00. | S99S99. | P99P99.T99T99. | | | | | | | |
| | | | | | F99F99. | | L99L99.E99E99. | | | | | | | |
| | | | | | | | V99V99.K99K99. | | | | | | | |
| 589 | 5 | TSFRVYG | LDSHG | NPGCFP | T99T99.S99S99. | L99L99.D99D99. | N99N99.P99P99. | 21 | 15 | 18 | 54 | Yes | Yes | Yes |
| | | | | | F99F99.R99R99. | S99S99.H99H99. | G99G99.C99C99. | | | | | | | |
| | | | | | V99V99.Y99Y99. | G99G99. | F99F99.P99P99. | | | | | | | |
| | | | | | G99G99. | | | | | | | | | |
| 591 | 5 | QLYGSHV | NEWVC | FPFEPASDTWEVF | Q99Q99.L99L99. | N99N99.E99E99. | F99F99.P99P99. | 21 | 15 | 39 | 75 | Yes | Yes | Yes |
| | | | | | Y99Y99.G99G99. | W99W99.V99V99. | F99F99.E99E99. | | | | | | | |
| | | | | | S99S99.H99H99. | C99C99. | P99P99.A99A99. | | | | | | | |
| | | | | | V99V99. | | S99S99.D99D99. | | | | | | | |
| | | | | | | | T99T99.W99W99. | | | | | | | |
| | | | | | | | E99E99.V99V99. | | | | | | | |
| | | | | | | | F99F99. | | | | | | | |
| 606 | 5 | YHAFG | VSTLL | HLWCPHSNSS | Y99Y99.H80B00. | V99V99.S99S99. | H99H99.L99L99. | 14 | 15 | 30 | 59 | Yes | Yes | Yes |
| | | | | | A99A99.F99F99. | T99T99.L99L99. | W99W99.C99C99. | | | | | | | |
| | | | | | G99G99. | L99L99. | P99P99.H99H99. | | | | | | | |
| | | | | | | | S99S99.N99N99. | | | | | | | |
| | | | | | | | S99S99.S99S99. | | | | | | | |
| 611 | 5 | LGVQSPA | PQSNR | PELHQWDWWYGRT | L99L99.G99G99. | P99P99.Q99Q99. | P99P99.E99E99. | 21 | 15 | 39 | 75 | Yes | Yes | Yes |
| | | | | | V99V99.Q99Q99. | S99S99.N99N99. | L99L99.H99H99. | | | | | | | |
| | | | | | S99S99.P99P99. | R99R99. | Q99Q99.W99W99. | | | | | | | |
| | | | | | A99A99. | | D99D99.W99W99. | | | | | | | |
| | | | | | | | W99W99.Y99Y99. | | | | | | | |
| | | | | | | | G99G99.R99R99. | | | | | | | |
| | | | | | | | T99T99. | | | | | | | |
| 612 | 5 | ILVSCGW | CYGEG | YMARIRAGCITVL | I80B00.L99L99. | C99C99.Y99Y99. | Y99Y99.M99M99. | 20 | 15 | 37 | 72 | Yes | Yes | Yes |
| | | | | | V99V99.S99S99. | G99G99.E99E99. | A99A99.R99R99. | | | | | | | |
| | | | | | C99C99.G99G99. | G99G99. | I99I99.R99R99. | | | | | | | |
| | | | | | W99W99. | | A80B00.G99G99. | | | | | | | |
| | | | | | | | C99C99.I99I99. | | | | | | | |
| | | | | | | | T99T99.V80B00. | | | | | | | |
| | | | | | | | L99L99. | | | | | | | |
| 620 | 5 | DVSWYCQ | PFGGL | LSSTRVAVLW | D99D99.V99V99. | P99P99.F99F99. | L99L99.S99S99. | 21 | 15 | 30 | 66 | Yes | Yes | Yes |
| | | | | | S99S99.W99W99. | G99G99.G99G99. | S99S99.T99T99. | | | | | | | |
| | | | | | Y99Y99.C99C99. | L99L99. | R99R99.V99V99. | | | | | | | |
| | | | | | Q99Q99. | | A99A99.V99V99. | | | | | | | |
| | | | | | | | L99L99.W99W99. | | | | | | | |
| 628 | 5 | QI | NAPSH | HFFDASGFH | Q99Q99.I99I99. | N99N99.A99A99. | H99H99.F99F99. | 6 | 15 | 27 | 48 | Yes | Yes | Yes |
| | | | | | | P99P99.S99S99. | F99F99.D99D99. | | | | | | | |
| | | | | | | H99H99. | A99A99.S99S99. | | | | | | | |
| | | | | | | | G99G99.F99F99. | | | | | | | |
| | | | | | | | H99H99. | | | | | | | |
| 643 | 5 | RICRCVW | QGPWG | SVLTQFGLVL | R99R99.I99I99. | Q99Q99.G99G99. | B00 | 21 | 15 | 0 | 36 | Yes | Yes | Yes |
| | | | | | C99C99.R99R99. | P99P99.W99W99. | | | | | | | | |
| | | | | | C99C99.V99V99. | G99G99. | | | | | | | | |
| | | | | | W99W99. | | | | | | | | | |
| 645 | 5 | SFVHLLL | NAAKT | YTYSIFGVV | S99S99.F99F99. | N99N99.A99A99. | Y99Y99.T99T99. | 21 | 15 | 27 | 63 | Yes | Yes | Yes |
| | | | | | V99V99.H99H99. | A99A99.K99K99. | Y99Y99.S99S99. | | | | | | | |
| | | | | | L99L99.L99L99. | T99T99. | I99I99.F99F99. | | | | | | | |
| | | | | | L99L99. | | G99G99.V99V99. | | | | | | | |
| | | | | | | | V99V99. | | | | | | | |
| 664 | 5 | GRATIRV | MWGDR | VTHTDPWGEL | G99G99.R99R99. | M99M99.W99W99. | V99V99.T99T99. | 21 | 15 | 30 | 66 | Yes | Yes | Yes |
| | | | | | A99A99.T99T99. | G99G99.D99D99. | H99H99.T99T99. | | | | | | | |
| | | | | | I99I99.R99R99. | R99R99. | D99D99.P99P99. | | | | | | | |
| | | | | | V99V99. | | W99W99.G99G99. | | | | | | | |
| | | | | | | | E99E99.L99L99. | | | | | | | |
| 665 | 5 | SLGGKIY | VSSVL | ITYDTISANR | S99S99.L99L99. | V99V99.S99S99. | I99I99.T99T99. | 21 | 15 | 30 | 66 | Yes | Yes | Yes |
| | | | | | G99G99.G99G99. | S99S99.V99V99. | Y99Y99.D99D99. | | | | | | | |
| | | | | | K99K99.I99I99. | L99L99. | T99T99.I99I99. | | | | | | | |
| | | | | | Y99Y99. | | S99S99.A99A99. | | | | | | | |
| | | | | | | | N99N99.R99R99. | | | | | | | |
| 678 | 5 | SNVLPQT | GSFID | DHNFTFAP | S99S99.N99N99. | G99G99.S99S99. | D80B00.H99H99. | 21 | 15 | 17 | 53 | Yes | Yes | Yes |
| | | | | | V99V99.L99L99. | F99F99.I99I99. | N80B00.F80B00. | | | | | | | |
| | | | | | P99P99.Q99Q99. | D99D99. | T80B00.F80B00. | | | | | | | |
| | | | | | T99T99. | | A80B00.P80B00. | | | | | | | |
| 688 | 5 | FWGTLMF | LSPIL | ICDYGV | F99F99.W99W99. | L99L99.S99S99. | I99I99.C99C99. | 21 | 15 | 18 | 54 | Yes | Yes | Yes |
| | | | | | G99G99.T99T99. | P99P99.I99I99. | D99D99.Y99Y99. | | | | | | | |
| | | | | | L99L99.M99M99. | L99L99. | G99G99.V99V99. | | | | | | | |
| | | | | | F99F99. | | | | | | | | | |
| 691 | 5 | SYVL | WTTSR | VSLTLR | S99S99.Y99Y99. | W99W99.T99T99. | V99V99.S99S99. | 12 | 15 | 18 | 45 | Yes | Yes | Yes |
| | | | | | V99V99.L99L99. | T99T99.S99S99. | L99L99.T99T99. | | | | | | | |
| | | | | | | R99R99. | L99L99.R99R99. | | | | | | | |
| 693 | 5 | SESAIWF | SHTFE | RAFYYN | S99S99.E99E99. | S99S99.H99H99. | R99R99.A99A99. | 21 | 15 | 18 | 54 | Yes | Yes | Yes |
| | | | | | S99S99.A99A99. | T99T99.F99F99. | F99F99.Y99Y99. | | | | | | | |
| | | | | | I99I99.W99W99. | E99E99. | Y99Y99.N99N99. | | | | | | | |
| | | | | | F99F99. | | | | | | | | | |
| 698 | 5 | LDSGVCR | NALHD | VCPTMGW | L99L99.D99D99. | N99N99.A99A99. | V99V99.C99C99. | 21 | 15 | 21 | 57 | Yes | Yes | Yes |
| | | | | | S99S99.G99G99. | L99L99.H99H99. | P99P99.T99T99. | | | | | | | |
| | | | | | V99V99.C99C99. | D99D99. | M99M99.G99G99. | | | | | | | |
| | | | | | R99R99. | | W99W99. | | | | | | | |
| 708 | 5 | NVVGQVF | QAGDP | VKWAHQVCQ | N99N99.V99V99. | Q99Q99.A99A99. | V99V99.K99K99. | 21 | 15 | 27 | 63 | Yes | Yes | Yes |
| | | | | | V99V99.G99G99. | G99G99.D99D99. | W99W99.A99A99. | | | | | | | |
| | | | | | Q99Q99.V99V99. | P99P99. | H99H99.Q99Q99. | | | | | | | |
| | | | | | F99F99. | | V99V99.C99C99. | | | | | | | |
| | | | | | | | Q99Q99. | | | | | | | |
| 713 | 5 | LLSEFIV | STTHL | LSVWCC | _80B00.L99L99. | S99S99.T99T99. | L99L99.S99S99. | 20 | 15 | 18 | 53 | Yes | Yes | Yes |
| | | | | | S99S99.E99E99. | T99T99.H99H99. | V99V99.W99W99. | | | | | | | |
| | | | | | F99F99.I99I99. | L99L99. | C99C99.C99C99. | | | | | | | |
| | | | | | V99V99. | | | | | | | | | |
| 720 | 5 | TTRNIWY | CNLLV | RFGISA | T99T99.T99T99. | C99C99.N99N99. | R99R99.F60A40. | 21 | 15 | 14 | 50 | Yes | Yes | Yes |
| | | | | | R99R99.N99N99. | L99L99.L99L99. | G60C40.I99I99. | | | | | | | |
| | | | | | I99I99.W99W99. | V99V99. | S60M40.A60C40. | | | | | | | |
| | | | | | Y99Y99. | | | | | | | | | |
| 721 | 5 | DIYHNLY | IDSAH | AYWSPLRQST | D99D99.I99I99. | I99I99.D99D99. | A99A99.Y99Y99. | 20 | 15 | 30 | 65 | Yes | Yes | Yes |
| | | | | | Y99Y99.H99H99. | S99S99.A99A99. | W99W99.S99S99. | | | | | | | |
| | | | | | N99N99.L99L99. | H99H99. | P99P99.L99L99. | | | | | | | |
| | | | | | Y80B00. | | R99R99.Q99Q99. | | | | | | | |
| | | | | | | | S99S99.T99T99. | | | | | | | |
| 730 | 5 | VVQIVRS | VLDSP | STRFRH | V99V99.V99V99. | V99V99.L99L99. | S99S99.T99T99. | 21 | 15 | 18 | 54 | Yes | Yes | Yes |
| | | | | | Q99Q99.I99I99. | D99D99.S99S99. | R99R99.F99F99. | | | | | | | |
| | | | | | V99V99.R99R99. | P99P99. | R99R99.H99H99. | | | | | | | |
| | | | | | S99S99. | | | | | | | | | |
| 742 | 5 | RVKYEVY | PACPP | ASLYGSKIC | R99R99.V99V99. | P99P99.A99A99. | A99A99.S99S99. | 21 | 15 | 27 | 63 | Yes | Yes | Yes |
| | | | | | K99K99.Y99Y99. | C99C99.P99P99. | L99L99.Y99Y99. | | | | | | | |
| | | | | | E99E99.V99V99. | P99P99. | G99G99.S99S99. | | | | | | | |
| | | | | | Y99Y99. | | K99K99.I99I99. | | | | | | | |
| | | | | | | | C99C99. | | | | | | | |
| 745 | 5 | ALSSQVF | PGIGI | LTLLSLSVLR | A99A99.L99L99. | P99P99.G99G99. | L99L99.T99T99. | 21 | 15 | 30 | 66 | Yes | Yes | Yes |
| | | | | | S99S99.S99S99. | I99I99.G99G99. | L99L99.L99L99. | | | | | | | |
| | | | | | Q99Q99.V99V99. | I99I99. | S99S99.L99L99. | | | | | | | |
| | | | | | F99F99. | | S99S99.V99V99. | | | | | | | |
| | | | | | | | L99L99.R99R99. | | | | | | | |
| 753 | 5 | GLLLGA | RVYAL | TTFDLSGKR | G99G99.L99L99. | R99R99.V99V99. | T99T99.T99T99. | 21 | 15 | 27 | 63 | Yes | Yes | Yes |
| | | | | | L99L99.L99L99. | Y99Y99.A99A99. | F99F99.D99D99. | | | | | | | |
| | | | | | G99G99.A99A99. | L99L99. | L99L99.S99S99. | | | | | | | |
| | | | | | I99I99. | | G99G99.K99K99. | | | | | | | |
| | | | | | | | R99R99. | | | | | | | |
| 754 | 5 | HILGLDG | WRVGG | SYDPWTMAY | H99H99.I99I99. | W99W99.R99R99. | S99S99.Y99Y99. | 21 | 15 | 27 | 63 | Yes | Yes | Yes |
| | | | | | L99L99.G99G99. | V99V99.G99G99. | D99D99.P99P99. | | | | | | | |
| | | | | | L99L99.D99D99. | G99G99. | W99W99.T99T99. | | | | | | | |
| | | | | | G99G99. | | M99M99.A99A99. | | | | | | | |
| | | | | | | | Y99Y99. | | | | | | | |
| 757 | 5 | CFKTIPC | NYITM | FLSVEA | C99C99.F99F99. | N99N99.Y99Y99. | F99F99.L99L99. | 21 | 15 | 18 | 54 | Yes | Yes | Yes |
| | | | | | K99K99.T99T99. | I99I99.T99T99. | S99S99.V99V99. | | | | | | | |
| | | | | | I99I99.P99P99. | M99M99. | E99E99.A99A99. | | | | | | | |
| | | | | | C99C99. | | | | | | | | | |
| 764 | 5 | QTANCVM | MDFEH | YAWKSLGCIV | Q80B00.T99T99. | M99M99.D99D99. | Y99Y99.A99A99. | 20 | 15 | 30 | 65 | Yes | Yes | Yes |
| | | | | | A99A99.N99N99. | F99F99.E99E99. | W99W99.K99K99. | | | | | | | |
| | | | | | C99C99.V99V99. | H99H99. | S99S99.L99L99. | | | | | | | |
| | | | | | M99M99 | | G99G99.C99C99. | | | | | | | |
| | | | | | | | I99I99.V99V99. | | | | | | | |
| 767 | 5 | VRVCGCS | FLTAY | SVYELHMLS | V99V99.R99R99. | F99F99.L99L99. | S99S99.V99V99. | 21 | 15 | 27 | 63 | Yes | Yes | Yes |
| | | | | | V99V99.C99C99. | T99T99.A99A99. | Y99Y99.E99E99. | | | | | | | |
| | | | | | G99G99.C99C99. | Y99Y99. | L99L99.H99H99. | | | | | | | |
| | | | | | S99S99. | | M99M99.L99L99. | | | | | | | |
| | | | | | | | S99S99. | | | | | | | |
| 769 | 5 | GVSLIYS | HVPGR | FVSSVM | G99G99.V99V99. | H99H99.V99V99. | F99F99.V99V99. | 21 | 15 | 18 | 54 | Yes | Yes | Yes |
| | | | | | S99S99.L99L99. | P99P99.G99G99. | S99S99.S99S99. | | | | | | | |
| | | | | | I99I99.Y99Y99. | R99R99. | V99V99.M99M99. | | | | | | | |
| | | | | | S99S99. | | | | | | | | | |
| 771 | 5 | LNSTCYA | GISLC | LVATSTRVCW | L99L99.N99N99. | G99G99.I99I99. | L99L99.V99V99. | 21 | 15 | 30 | 66 | Yes | Yes | Yes |
| | | | | | S99S99.T99T99. | S99S99.L99L99. | A99A99.T99T99. | | | | | | | |
| | | | | | C99C99.Y99Y99. | C99C99. | S99S99.T99T99. | | | | | | | |
| | | | | | A99A99. | | R99R99.V99V99. | | | | | | | |
| | | | | | | | C99C99.W99W99. | | | | | | | |
| 782 | 5 | SSDVT | LSGQW | QYIEC | S99S99.S99S99. | L99L99.S99S99. | Q99Q99.Y99Y99. | 21 | 15 | 15 | 51 | Yes | Yes | Yes |
| | | | | | L99L99.V99V99. | G99G99.Q99Q99. | I99I99.E99E99. | | | | | | | |
| | | | | | D99D99.V99V99. | W99W99. | C99C99. | | | | | | | |
| | | | | | T99T99. | | | | | | | | | |
| 786 | 5 | GFSCTVY | KNDTL | CVWDKFNVL | G99G99.F99F99. | K99K99.N99N99. | C99C99.V99V99. | 21 | 15 | 27 | 63 | Yes | Yes | Yes |
| | | | | | S99S99.C99C99. | D99D99.T99T99. | W99W99.D99D99. | | | | | | | |
| | | | | | T99T99.V99V99. | L99L99. | K99K99.F99F99. | | | | | | | |
| | | | | | Y99Y99. | | N99N99.V99V99. | | | | | | | |
| | | | | | | | L99L99. | | | | | | | |
| 79 | 5 | GIRCLIH | GSRAW | LIEHVDSSSF | G99G99.I99I99. | G99G99.S99S99. | L99L99.I99I99. | 21 | 15 | 30 | 66 | Yes | Yes | Yes |
| | | | | | R99R99.C99C99. | R99R99.A99A99. | E99E99.H99H99. | | | | | | | |
| | | | | | L99L99.I99I99. | W99W99. | V99V99.D99D99. | | | | | | | |
| | | | | | H99H99. | | S99S99.S99S99. | | | | | | | |
| | | | | | | | S99S99.F99F99. | | | | | | | |
| 801 | 5 | FLFVRCF | SRGSA | TYTMFSRSAC | F99F99.L99L99. | S99S99.R99R99. | T99T99.Y99Y99. | 21 | 15 | 30 | 66 | Yes | Yes | Yes |
| | | | | | F99F99.V99V99. | G99G99.S99S99. | T99T99.M99M99. | | | | | | | |
| | | | | | R99R99.C99C99. | A99A99. | F99F99.S99S99. | | | | | | | |
| | | | | | F99F99. | | R99R99.S99S99. | | | | | | | |
| | | | | | | | A99A99.C99C99. | | | | | | | |
| 807 | 5 | CQSYSWP | GECGF | RTWGLMGRM | C99C99.Q99Q99. | G99G99.E99E99. | R99R99.T99T99. | 21 | 15 | 27 | 63 | Yes | Yes | Yes |
| | | | | | S99S99.Y99Y99. | C99C99.G99G99. | W99W99.G99G99. | | | | | | | |
| | | | | | S99S99.W99W99. | F99F99. | L99L99.M99M99. | | | | | | | |
| | | | | | P99P99. | | G99G99.R99R99. | | | | | | | |
| | | | | | | | M99M99. | | | | | | | |
| 808 | 5 | GLTGMTI | PGDAA | YYYNLHSLI | G99G99.L99L99. | P99P99.G99G99. | Y99Y99.Y99Y99. | 21 | 15 | 27 | 63 | Yes | Yes | Yes |
| | | | | | T99T99.G99G99. | D99D99.A99A99. | Y99Y99.N99N99. | | | | | | | |
| | | | | | M99M99.T99T99. | A99A99. | L99L99.H99H99. | | | | | | | |
| | | | | | I99I99. | | S99S99.L99L99. | | | | | | | |
| | | | | | | | I99I99. | | | | | | | |
| 817 | 5 | QLSRTGY | TGPVG | IDSYGR | Q99Q99.L99L99. | T99T99.G99G99. | I99I99.D99D99. | 21 | 15 | 18 | 54 | Yes | Yes | Yes |
| | | | | | S99S99.R99R99. | P99P99.V99V99. | S99S99.Y99Y99. | | | | | | | |
| | | | | | T99T99.G99G99. | G99G99. | G99G99.R99R99. | | | | | | | |
| | | | | | Y99Y99. | | | | | | | | | |
| 819 | 5 | LAWHFCC | VTRSS | STFESHPFVR | L99L99.A99A99. | V99V99.T99T99. | S99S99.T99T99. | 21 | 15 | 30 | 66 | Yes | Yes | Yes |
| | | | | | W99W99.H99H99. | R99R99.S99S99. | F99F99.E99E99. | | | | | | | |
| | | | | | F99F99.C99C99. | S99S99. | S99S99.H99H99. | | | | | | | |
| | | | | | C99C99. | | P99P99.F99F99. | | | | | | | |
| | | | | | | | V99V99.R99R99. | | | | | | | |
| 820 | 5 | VTCMWST | MRYDN | CHGGWM | V99V99.T99T99. | M99M99.R99R99. | C99C99.H99H99. | 21 | 15 | 18 | 54 | Yes | Yes | Yes |
| | | | | | C99C99.M99M99. | Y99Y99.D99D99. | G99G99.G99G99. | | | | | | | |
| | | | | | W99W99.S99S99. | N99N99. | W99W99.M99M99. | | | | | | | |
| | | | | | T99T99. | | | | | | | | | |
| 823 | 5 | LIVSVST | YTRGF | DPDRRV | L99L99.I99I99. | Y99Y99.T99T99. | D99D99.P99P99. | 21 | 15 | 18 | 54 | Yes | Yes | Yes |
| | | | | | V99V99.S99S99. | R99R99.G99G99. | D99D99.R99R99. | | | | | | | |
| | | | | | V99V99.S99S99. | F99F99. | R99R99.V99V99. | | | | | | | |
| | | | | | T99T99. | | | | | | | | | |
| 826 | 5 | NAFFGLL | TLSCD | WLMHPDCLVV | N99N99.A99A99. | T99T99.L99L99. | W99W99.L99L99. | 21 | 15 | 30 | 66 | Yes | Yes | Yes |
| | | | | | F99F99.F99F99. | S99S99.C99C99. | M99M99.H99H99. | | | | | | | |
| | | | | | G99G99.L99L99. | D99D99. | P99P99.D99D99. | | | | | | | |
| | | | | | L99L99. | | C99C99.L99L99. | | | | | | | |
| | | | | | | | V99V99.V99V99. | | | | | | | |
| 834 | 5 | DVKWLT | ARTFH | LCLNW | D99D99.V99V99. | A99A99.R99R99. | L99L99.C99C99. | 18 | 15 | 15 | 48 | Yes | Yes | Yes |
| | | | | | K99K99.W99W99. | T99T99.F99F99. | L99L99.N99N99. | | | | | | | |
| | | | | | L99L99.T99T99. | H99H99. | W99W99. | | | | | | | |
| 836 | 5 | YGVSHSC | TISEL | LGYIGLFGF | Y99Y99.G99G99. | T99T99.I99I99. | L99L99.G99G99. | 21 | 15 | 27 | 63 | Yes | Yes | Yes |
| | | | | | V99V99.S99S99. | S99S99.E99E99. | Y99Y99.I99I99. | | | | | | | |
| | | | | | H99H99.S99S99. | L99L99. | G99G99.L99L99. | | | | | | | |
| | | | | | C99C99. | | F99F99.G99G99. | | | | | | | |
| | | | | | | | F99F99. | | | | | | | |
| 840 | 5 | DDWCSCT | LFAPF | QSLNNNGFFS | D99D99.D80B00. | L99L99.F99F99. | Q99Q99.S99S99. | 20 | 15 | 30 | 65 | Yes | Yes | Yes |
| | | | | | W99W99.C99C99. | A99A99.P99P99. | L99L99.N99N99. | | | | | | | |
| | | | | | S99S99.C99C99. | F99F99. | N99N99.N99N99. | | | | | | | |
| | | | | | T99T99. | | G99G99.F99F99. | | | | | | | |
| | | | | | | | F99F99.S99S99. | | | | | | | |
| 852 | 5 | CCVLQMW | PHGYR | DLLNDVVHSA | C99C99.C99C99. | P99P99.H99H99. | D99D99.L99L99. | 21 | 15 | 30 | 66 | Yes | Yes | Yes |
| | | | | | V99V99.L99L99. | G99G99.Y99Y99. | L99L99.N99N99. | | | | | | | |
| | | | | | Q99Q99.M99M99. | R99R99. | D99D99.V99V99. | | | | | | | |
| | | | | | W99W99. | | V99V99.H99H99. | | | | | | | |
| | | | | | | | S99S99.A99A99. | | | | | | | |
| 853 | 5 | SSSDFVV | VVPGV | VSRDANADM | S99S99.S99S99. | V99V99.V99V99. | B00 | 21 | 15 | 0 | 36 | Yes | Yes | Yes |
| | | | | | S99S99.D99D99. | P99P99.G99G99. | | | | | | | | |
| | | | | | F99F99.V99V99. | V99V99. | | | | | | | | |
| | | | | | V99V99. | | | | | | | | | |
| 856 | 5 | VQSEHRV | LACAI | SFRVGHAGFG | V99V99.Q99Q99. | L99L99.A99A99. | S99S99.F99F99. | 21 | 15 | 30 | 66 | Yes | Yes | Yes |
| | | | | | S99S99.E99E99. | C99C99.A99A99. | R99R99.V99V99. | | | | | | | |
| | | | | | H99H99.R99R99. | I99I99. | G99G99.H99H99. | | | | | | | |
| | | | | | V99V99. | | A99A99.G99G99. | | | | | | | |
| | | | | | | | F99F99.G99G99. | | | | | | | |
| 858 | 5 | YNSQIVW | CGCRI | ALFRPREDVI | Y99Y99.N99N99. | C99C99.G99G99. | A99A99.L99L99. | 21 | 15 | 30 | 66 | Yes | Yes | Yes |
| | | | | | S99S99.Q99Q99. | C99C99.R99R99. | F99F99.R99R99. | | | | | | | |
| | | | | | I99I99.V99V99. | I99I99. | P99P99.R99R99. | | | | | | | |
| | | | | | W99W99. | | E99E99.D99D99. | | | | | | | |
| | | | | | | | V99V99.I99I99. | | | | | | | |
| 859 | 5 | LFTGAWI | GVAQS | SMCRRENMLC | L99L99.F99F99. | G99G99.V99V99. | S99S99.M99M99. | 21 | 15 | 30 | 66 | Yes | Yes | Yes |
| | | | | | T99T99.G99G99. | A99A99.Q99Q99. | C99C99.R99R99. | | | | | | | |
| | | | | | A99A99.W99W99. | S99S99. | R99R99.E99E99. | | | | | | | |
| | | | | | I99I99. | | N99N99.M99M99. | | | | | | | |
| | | | | | | | L99L99.C99C99. | | | | | | | |
| 860 | 5 | RLDRLVK | CYGVL | WHFLTHDKII | R99R99.L99L99. | C99C99.Y99Y99. | W99W99.H99H99. | 21 | 15 | 30 | 66 | Yes | Yes | Yes |
| | | | | | D99D99.R99R99. | G99G99.V99V99. | F99F99.L99L99. | | | | | | | |
| | | | | | L99L99.V99V99. | L99L99. | T99T99.H99H99. | | | | | | | |
| | | | | | K99K99. | | D99D99.K99K99. | | | | | | | |
| | | | | | | | I99I99.I99I99. | | | | | | | |
| 866 | 5 | VVGILRG | CYRDA | YSRFVITPFG | V99V99.V99V99. | C99C99.Y99Y99. | Y99Y99.S99S99. | 21 | 15 | 30 | 66 | Yes | Yes | Yes |
| | | | | | G99G99.I99I99. | R99R99.D99D99. | R99R99.F99F99. | | | | | | | |
| | | | | | L99L99.R99R99. | A99A99. | V99V99.I99I99. | | | | | | | |
| | | | | | G99G99. | | T99T99.P99P99. | | | | | | | |
| | | | | | | | F99F99.G99G99. | | | | | | | |
| 868 | 5 | SPSLLCS | MPYWA | VEPHLYDLWS | S99S99.P99P99. | M99M99.P99P99. | V99V99.E99E99. | 21 | 15 | 30 | 66 | Yes | Yes | Yes |
| | | | | | S99S99.L99L99. | Y99Y99.W99W99. | P99P99.H99H99. | | | | | | | |
| | | | | | L99L99.C99C99. | A99A99. | L99L99.Y99Y99. | | | | | | | |
| | | | | | S99S99. | | D99D99.L99L99. | | | | | | | |
| | | | | | | | W99W99.S99S99. | | | | | | | |
| 869 | 5 | TFNFGLR | ACDPPL | AFTTLSKYAS | T99T99.F99F99. | A99A99.C99C99. | A99A99.F99F99. | 21 | 15 | 30 | 66 | Yes | Yes | Yes |
| | | | | | N99N99.F99F99. | D99D99.P99P99. | T99T99.T99T99. | | | | | | | |
| | | | | | G99G99.L99L99. | L99L99. | L99L99.S99S99. | | | | | | | |
| | | | | | R99R99. | | K99K99.Y99Y99. | | | | | | | |
| | | | | | | | A99A99.S99S99. | | | | | | | |
| 871 | 5 | GRYGVVW | SQCGV | SIFHHPYVY | G99G99.R99R99. | S99S99.Q99Q99. | S99S99.I99I99. | 21 | 15 | 27 | 63 | Yes | Yes | Yes |
| | | | | | Y99Y99.G99G99. | C99C99.G99G99. | F99F99.H99H99. | | | | | | | |
| | | | | | V99V99.V99V99. | V99V99. | H99H99.P99P99. | | | | | | | |
| | | | | | W99W99. | | Y99Y99.V99V99. | | | | | | | |
| | | | | | | | Y99Y99. | | | | | | | |
| 875 | 5 | VADGIVY | YASVS | WRYVSGDIL | V99V99.A99A99. | Y99Y99.A99A99. | W99W99.R99R99. | 21 | 14 | 27 | 62 | Yes | Yes | Yes |
| | | | | | D99D99.G99G99. | S99S99.V99V99. | Y99Y99.V99V99. | | | | | | | |
| | | | | | I99I99.V99V99. | S80B00. | S99S99.G99G99. | | | | | | | |
| | | | | | Y99Y99. | | D99D99.I99I99. | | | | | | | |
| | | | | | | | L99L99. | | | | | | | |
| 877 | 5 | T | DGG | WASFVD | T99T99. | R99R99.T99T99. | W99W99.A99A99. | 3 | 15 | 18 | 36 | Yes | Yes | Yes |
| | | | | | | D99D99.G99G99. | S99S99.F99F99. | | | | | | | |
| | | | | | | G99G99. | V99V99.D99D99. | | | | | | | |
| 878 | 5 | MLCANTY | LSHTV | MSCIG | M99M99.L99L99. | L99L99.S99S99. | M99M99.S99S99. | 21 | 15 | 15 | 51 | Yes | Yes | Yes |
| | | | | | C99C99.A99A99. | H99H99.T99T99. | C99C99.I99I99. | | | | | | | |
| | | | | | N99N99.T99T99. | V99V99. | G99G99. | | | | | | | |
| | | | | | Y99Y99. | | | | | | | | | |
| 887 | 5 | VV | EYGYR | LTQDTRRCSW | V99V99.V99V99. | E99E99.Y99Y99. | L99L99.T99T99. | 6 | 15 | 30 | 51 | Yes | Yes | Yes |
| | | | | | | G99G99.Y99Y99. | Q99Q99.D99D99. | | | | | | | |
| | | | | | | R99R99. | T99T99.R99R99. | | | | | | | |
| | | | | | | | R99R99.C99C99. | | | | | | | |
| | | | | | | | S99S99.W99W99. | | | | | | | |
| 888 | 5 | FVFLVQE | CARQT | NSVGVIMTCWWRT | F99F99.V80B00. | C99C99.A99A99. | N99N99.S99S99. | 20 | 15 | 39 | 74 | Yes | Yes | Yes |
| | | | | | F99F99.L99L99. | R99R99.Q99Q99. | V99V99.G99G99. | | | | | | | |
| | | | | | V99V99.Q99Q99. | T99T99. | V99V99.I99I99. | | | | | | | |
| | | | | | E99E99. | | M99M99.T99T99. | | | | | | | |
| | | | | | | | C99C99.W99W99. | | | | | | | |
| | | | | | | | W99W99.R99R99. | | | | | | | |
| | | | | | | | T99T99. | | | | | | | |
| 889 | 5 | GFGTVLF | PARAW | CTTDLF | G99G99.F99F99. | P99P99.A99A99. | C99C99.T99T99. | 21 | 15 | 18 | 54 | Yes | Yes | Yes |
| | | | | | G99G99.T99T99. | R99R99.A99A99. | T99T99.D99D99. | | | | | | | |
| | | | | | V99V99.L99L99. | W99W99. | L99L99.F99F99. | | | | | | | |
| | | | | | F99F99. | | | | | | | | | |
| 891 | 5 | ILKIVFG | LDDIR | HIFDSF | I99I99.L99L99. | L99L99.D99D99. | H99H99.I99I99. | 21 | 15 | 18 | 54 | Yes | Yes | Yes |
| | | | | | K99K99.H99H99. | D99D99.I99I99. | F99F99.D99D99. | | | | | | | |
| | | | | | V99V99.F99F99. | R99R99. | S99S99.F99F99. | | | | | | | |
| | | | | | G99G99. | | | | | | | | | |
| 896 | 5 | LCTGPAWVV | LRWPG | NHPSHTCVMC | L99L99.C99C99. | L99L99.R99R99. | N99N99.H80B00. | 21 | 15 | 29 | 65 | Yes | Yes | Yes |
| | | | | | T99T99.G99G99. | W99W99.P99P99. | P99P99.S99S99. | | | | | | | |
| | | | | | P99P99.A99A99. | G99G99. | H99H99.T99T99. | | | | | | | |
| | | | | | W99W99. | | C99C99.V99V99. | | | | | | | |
| | | | | | | | M99M99.C99C99. | | | | | | | |
| 899 | 5 | GGGSWFP | IGSSL | IMYASDFWVF | G99G99.G99G99. | I99I99.G99G99. | I60B00.M60B00. | 20 | 15 | 20 | 55 | Yes | Yes | Yes |
| | | | | | G99G99.S99S99. | S99S99.S99S99. | Y60B00.A60B00. | | | | | | | |
| | | | | | W99W99.F80B00. | L99L99. | S60Y40.D60B00. | | | | | | | |
| | | | | | P99P99. | | F60B00.W60B00. | | | | | | | |
| | | | | | | | V60B00.F60B00. | | | | | | | |
| 900 | 5 | LSLRNVF | NAGVH | AYFVDGIVIIT | L99L99.S99S99. | N99N99.A99A99. | A99A99.Y99Y99. | 20 | 15 | 27 | 62 | Yes | Yes | Yes |
| | | | | | L99L99.R99R99. | G99G99.V99V99. | F99F99.V99V99. | | | | | | | |
| | | | | | N80B00.V99V99. | H99H99. | D99D99.G99G99. | | | | | | | |
| | | | | | F99F99. | | I99I99.I99I99. | | | | | | | |
| | | | | | | | T99T99. | | | | | | | |
| 902 | 5 | ACFGLVY | GSQSQ | VYFRSMFCVH | A99A99.C99C99. | G99G99.S99S99. | V99V99.Y99Y99. | 21 | 15 | 30 | 66 | Yes | Yes | Yes |
| | | | | | F99F99.G99G99. | Q99Q99.S99S99. | F99F99.R99R99. | | | | | | | |
| | | | | | L99L99.V99V99. | Q99Q99. | S99S99.M99M99. | | | | | | | |
| | | | | | Y99Y99. | | F99F99.C99C99. | | | | | | | |
| | | | | | | | V99V99.H99H99. | | | | | | | |
| 903 | 5 | RVVR | SEAPK | FVQCTL | R99R99.V99V99. | S99S99.E99E99. | F99F99.V99V99. | 12 | 15 | 18 | 45 | Yes | Yes | Yes |
| | | | | | V99V99.R99R99. | A99A99.P99P99. | Q99Q99.C99C99. | | | | | | | |
| | | | | | | K99K99. | T99T99.L99L99. | | | | | | | |
| 904 | 5 | SLYAVVW | LCGYD | TCFVNHMKIS | S99S99.L99L99. | L99L99.C99C99. | T99T99.C99C99. | 21 | 15 | 30 | 66 | Yes | Yes | Yes |
| | | | | | Y99Y99.A99A99. | G99G99.Y99Y99. | F99F99.V99V99. | | | | | | | |
| | | | | | V99V99.V99V99. | D99D99. | N99N99.H99H99. | | | | | | | |
| | | | | | W99W99. | | M99M99.K99K99. | | | | | | | |
| | | | | | | | I99I99.S99S99. | | | | | | | |
| 905 | 5 | NPDPCVV | AYSAV | TYVSHPCHLY | N99N99.P99P99. | A99A99.Y99Y99. | T99T99.Y99Y99. | 21 | 15 | 30 | 66 | Yes | Yes | Yes |
| | | | | | D99D99.P99P99. | S99S99.A99A99. | V99V99.S99S99. | | | | | | | |
| | | | | | C99C99.V99V99. | V99V99. | H99H99.P99P99. | | | | | | | |
| | | | | | V99V99. | | C99C99.H99H99. | | | | | | | |
| | | | | | | | L99L99.Y99Y99. | | | | | | | |
| 906 | 5 | GHWCSLT | LAQYG | AYATYNLDRGYSM | G99G99.H99H99. | L99L99.A80B00. | A99A99.Y99Y99. | 21 | 14 | 39 | 74 | Yes | Yes | Yes |
| | | | | | W99W99.C99C99. | Q99Q99.Y99Y99. | A99A99.T99T99. | | | | | | | |
| | | | | | S99S99.L99L99. | G99G99. | Y99Y99.N99N99. | | | | | | | |
| | | | | | T99T99. | | L99L99.D99D99. | | | | | | | |
| | | | | | | | R99R99.G99G99. | | | | | | | |
| | | | | | | | Y99Y99.S99S99. | | | | | | | |
| | | | | | | | M99M99. | | | | | | | |
| 910 | 5 | VSFHVFY | NSGTH | VSIVQGTTG | V99V99.S99S99. | N99N99.S99S99. | V99V99.S99S99. | 21 | 15 | 27 | 63 | Yes | Yes | Yes |
| | | | | | F99F99.H99H99. | G99G99.T99T99. | I99I99.V99V99. | | | | | | | |
| | | | | | V99V99.F99F99. | H99H99. | Q99Q99.G99G99. | | | | | | | |
| | | | | | Y99Y99. | | T99T99.T99T99. | | | | | | | |
| | | | | | | | G99G99. | | | | | | | |
| 912 | 5 | MILVAGW | DVGDG | LITDTAGGSF | M99M99.I99I99. | D99D99.V99V99. | B00 | 21 | 15 | 0 | 36 | Yes | Yes | Yes |
| | | | | | L99L99.V99V99. | G99G99.D99D99. | | | | | | | | |
| | | | | | A99A99.G99G99. | G99G99. | | | | | | | | |
| | | | | | W99W99. | | | | | | | | | |
| 917 | 5 | LDPRA | LYAAY | FCATYQETCY | L99L99.D99D99. | L99L99.Y99Y99. | F99F99.C99C99. | 15 | 15 | 30 | 60 | Yes | Yes | Yes |
| | | | | | P99P99.R99R99. | A99A99.A99A99. | A99A99.T99T99. | | | | | | | |
| | | | | | A99A99. | Y99Y99. | Y99Y99.Q99Q99. | | | | | | | |
| | | | | | | | E99E99.T99T99. | | | | | | | |
| | | | | | | | C99C99.Y99Y99. | | | | | | | |
| 918 | 5 | RSHDCIN | GFP | PVVLRRNII | R99R99.S99S99. | G99G99.F99F99. | P99P99.V99V99. | 21 | 15 | 27 | 63 | Yes | Yes | Yes |
| | | | | | H99H99.D99D99. | P99P99.I99I99. | V99V99.L99L99. | | | | | | | |
| | | | | | C99C99.I99I99. | I99I99. | R99R99.R99R99. | | | | | | | |
| | | | | | N99N99. | | N99N99.I99I99. | | | | | | | |
| | | | | | | | I99I99. | | | | | | | |
| 919 | 5 | NWSCVFQ | QSPAV | LACSLTGIC | N99N99.W99W99. | Q99Q99.S99S99. | L99L99.A99A99. | 20 | 15 | 27 | 62 | Yes | Yes | Yes |
| | | | | | S80B00.C99C99. | P99P99.A99A99. | C99C99.S99S99. | | | | | | | |
| | | | | | V99V99.F99F99. | V99V99. | L99L99.T99T99. | | | | | | | |
| | | | | | Q99Q99. | | G99G99.I99I99. | | | | | | | |
| | | | | | | | C99C99. | | | | | | | |
| 689 | 6 | VLVCYSY | ASYR | LTSPLSDSS | V83B00.L83B00. | A66B00.S66B00. | B00 | 20 | 6 | 0 | 26 | No | Yes | Yes |
| | | | | | V66B00.C83B00. | Y50B00.R50B00. | | | | | | | | |
| | | | | | Y99Y99.S83B00. | | | | | | | | | |
| | | | | | Y83B00. | | | | | | | | | |
| 222 | 20 | RLICDMQ | FGD | GMVRDWSTRLSSE | R99R99.L99L99. | F99F99.G99G99. | G80B00.M80B00. | 21 | 9 | 27 | 57 | Yes | No | Yes |
| | | | | | I99I99.C99C99. | D99D99. | V80B00.R80B00. | | | | | | | |
| | | | | | D99D99.M95B00. | | D80B00.W80B00. | | | | | | | |
| | | | | | Q99Q99. | | S80B00.T80B00. | | | | | | | |
| | | | | | | | R90B00.L80B00. | | | | | | | |
| | | | | | | | S80B00.S80B00. | | | | | | | |
| | | | | | | | E80B00. | | | | | | | |
| 212 | 14 | HLVAT | ASYRYY | WPSLPTGGTP | B00 | A50B00.S50B00. | W99W99.P99P99. | 0 | 7 | 30 | 37 | Yes | No | Yes |
| | | | | | | Y50B00.R64B00. | S99S99.L99L99. | | | | | | | |
| | | | | | | Y57B00. | P99P99.T99T99. | | | | | | | |
| | | | | | | | G99G99.G99G99. | | | | | | | |
| | | | | | | | T99T99.P99P99. | | | | | | | |
| 456 | 8 | VLVCYSY | ASYRYY | SNHVPSEPKPSVF | B00 | A62B00.S50B00. | S99S99.N99N99. | 0 | 6 | 39 | 45 | Yes | No | Yes |
| | | | | | | Y50B00.R50B00. | H99H99.V99V99. | | | | | | | |
| | | | | | | Y37B00. | P99P99.S99S99. | | | | | | | |
| | | | | | | | E99E99.P99P99. | | | | | | | |
| | | | | | | | K99K99.P99P99. | | | | | | | |
| | | | | | | | S99S99.V99V99. | | | | | | | |
| | | | | | | | F99F99. | | | | | | | |
| 4 | 32009 | VLVCYSY | ASYRY | LTSPLSDSS | V99B00.L99B00. | A99B00.S99B00. | B00 | 21 | 15 | 0 | 36 | No | No | Yes |
| | | | | | V99B00.C99B00. | Y99B00.R99B00. | | | | | | | | |
| | | | | | Y99B00.S99B00. | Y99B00. | | | | | | | | |
| | | | | | Y99B00. | | | | | | | | | |
| 80 | 166 | LVVTYER | ATLRG | LIPAESGLVPLVD | L98B00.V99B00. | A99A99.T99T99. | B00 | 21 | 15 | 0 | 36 | No | No | Yes |
| | | | | | V99V99.T98B00. | L99L99.R99R99. | | | | | | | | |
| | | | | | Y99B00.E99B00. | G99B00. | | | | | | | | |
| | | | | | R99B00. | | | | | | | | | |
| 16 | 112 | VCVSYTH | ETWMH | MRCPPHFFV | V99B00.C99B00. | E99E99.T99B00. | B00 | 21 | 15 | 0 | 36 | No | No | Yes |
| | | | | | V98B00.S99B00. | W99B00.M99B00. | | | | | | | | |
| | | | | | Y99B00.T97B00. | H99B00. | | | | | | | | |
| | | | | | H98B00. | | | | | | | | | |
| 216 | 111 | ILCMYEQ | CNART | AHLV | I97B00.L99L99. | C99B00.N99N99. | B00 | 21 | 15 | 0 | 36 | No | No | Yes |
| | | | | | C99C99.M99M99. | A99A99.R99R99. | | | | | | | | |
| | | | | | Y99Y99.E99E99. | T99B00. | | | | | | | | |
| | | | | | Q94B00. | | | | | | | | | |
| 161 | 48 | IYNCKST | CGVPF | TTDSVT | I97B00.Y93B00. | C99C99.G99G99. | T64B00.T66B00. | 21 | 15 | 12 | 48 | No | No | Yes |
| | | | | | N95B00.C99C99. | V99V99.P99P99. | D64S35.S64B00. | | | | | | | |
| | | | | | K97B00.S99S99. | F99F99. | V66B00.T64B00. | | | | | | | |
| | | | | | T97B00. | | | | | | | | | |
| 68 | 36 | LLMAYEC | GCHLV | YLIRHA | L99L99.L99L99. | G99G99.C99C99. | B00 | 21 | 15 | 0 | 36 | No | No | Yes |
| | | | | | M99M99.A99A99. | H99H99.L99L99. | | | | | | | | |
| | | | | | Y99Y99.E97B00. | V99V99. | | | | | | | | |
| | | | | | C99C99. | | | | | | | | | |
| 40 | 29 | LRVCYTR | ANYMN | SHYTALVCNV | L99L99.R89B00. | A99A99.N99N99. | B00 | 21 | 15 | 0 | 36 | No | No | Yes |
| | | | | | V99V99.C99C99. | Y99Y99.M99M99. | | | | | | | | |
| | | | | | Y99Y99.T99T99. | N99N99. | | | | | | | | |
| | | | | | R99R99. | | | | | | | | | |
| 181 | 18 | IPVFYC | PGFNL | CNCNAPSFVQ | I99I99.P99P99. | P99P99.G99G99. | B00 | 21 | 15 | 0 | 36 | No | No | Yes |
| | | | | | V99V99.F99F99. | F99F99.N99N99. | | | | | | | | |
| | | | | | Y99Y99.C99C99. | L99L99. | | | | | | | | |
| | | | | | I94B00. | | | | | | | | | |
| 140 | 15 | FLCCYGI | ANL | LGPHVP | F99F99.L99L99. | A99A99.N99N99. | B00 | 21 | 15 | 0 | 36 | No | No | Yes |
| | | | | | C99C99.C99C99. | L99L99.N99N99. | | | | | | | | |
| | | | | | Y99Y99.G99G99. | R99R99. | | | | | | | | |
| | | | | | I99I99. | | | | | | | | | |
| 229 | 9 | DYGYCTF | YPGCH | LLSRPKRCFVMVM | D99D99.Y99Y99. | Y99Y99.P99P99. | B00 | 21 | 15 | 0 | 36 | No | No | Yes |
| | | | | | G99G99.Y99Y99. | G99G99.C99C99. | | | | | | | | |
| | | | | | C99C99.T99T99. | H99H99. | | | | | | | | |
| | | | | | F99F99. | | | | | | | | | |
| 654 | 7 | RP | AYGCV | NFQASN | R99R99.P99P99. | A99A99.Y99Y99. | N99N99.F99F99. | 6 | 15 | 18 | 39 | No | No | Yes |
| | | | | | | G99G99.C99C99. | Q99Q99.A99A99. | | | | | | | |
| | | | | | | V99V99. | S99S99.N99N99. | | | | | | | |
| 38 | 9 | TKFEGLV | YFLQH | SC | T99T99.K99K99. | Y99Y99.F99F99. | B00 | 21 | 15 | 0 | 36 | No | No | Yes |
| | | | | | F99F99.E99E99. | L99L99.Q99Q99. | | | | | | | | |
| | | | | | G99G99.L99L99. | H99H99. | | | | | | | | |
| | | | | | V99V99. | | | | | | | | | |
| 855 | 5 | VNLLVCA | NGWHM | LTSPLSDSS | T40B00.G60B00. | A40B00.V60B00. | L99L99.T99T99. | 9 | 6 | 27 | 42 | Yes | Yes | No |
| | | | | | G40L40.L40B00. | W40B00.A40B00. | S99S99.P99P99. | | | | | | | |
| | | | | | C80B00.B00B00. | B00B00. | L99L99.S99S99. | | | | | | | |
| | | | | | S40B00. | | D99D99.S99S99. | | | | | | | |
| | | | | | | | S99S99. | | | | | | | |
| 511 | 20 | SSRSRFN | ASYRY | LTSPLSDSS | B00B00.B00B00. | A90B00.S95B00. | L95B00.T95B00. | 7 | 15 | 27 | 49 | Yes | No | No |
| | | | | | B00B00.B00B00. | Y95B00.R95B00. | S99S99.P95B00. | | | | | | | |
| | | | | | B00B00.B00B00. | Y90B00. | L90B00.S90B00. | | | | | | | |
| | | | | | B00B00. | | D90B00.S90B00. | | | | | | | |
| | | | | | | | S90B00. | | | | | | | |
| 10 | 13312 | SSRSRFN | GTGYE | YLMVTG | S97B00.S99B00. | G99B00.T99B00. | Y99B00.L99B00. | 21 | 15 | 18 | 54 | No | No | No |
| | | | | | R99B00.S99B00. | G99B00.Y99B00. | M99B00.V99B00. | | | | | | | |
| | | | | | R99B00.F99B00. | E99B00. | T99B00.G99B00. | | | | | | | |
| | | | | | N97B00. | | | | | | | | | |
| 0 | 2960 | SYVNVWG | GYAIS | TIYGVY | S99B00.Y98B00. | G99B00.Y99B00. | T99B00.I99B00. | 21 | 15 | 18 | 54 | No | No | No |
| | | | | | V99B00.N99B00. | A99B00.I99B00. | Y99B00.G99B00. | | | | | | | |
| | | | | | V99B00.W99B00. | S99B00. | V99B00.Y99B00. | | | | | | | |
| | | | | | G99B00. | | | | | | | | | |
| 1 | 2873 | GNVFLCG | FHTCG | WPILSA | G99B00.N98B00. | F99B00.H99B00. | W99B00.P99B00. | 21 | 15 | 18 | 54 | No | No | No |
| | | | | | V99B00.F99B00. | T99B00.C99B00. | I99B00.L99B00. | | | | | | | |
| | | | | | L99B00.C99B00. | G99G99. | S99B00.A99B00. | | | | | | | |
| | | | | | G99B00. | | | | | | | | | |
| 6 | 2527 | LRSRVIT | DVVSF | MTSEVT | L99B00.R99B00. | D99B00.V99B00. | M99B00.T99B00. | 21 | 15 | 18 | 54 | No | No | No |
| | | | | | S98B00.R99B00. | V99B00.S99B00. | S99B00.E99B00. | | | | | | | |
| | | | | | V99B00.I99B00. | F99B00. | V99B00.T99B00. | | | | | | | |
| | | | | | T98B00. | | | | | | | | | |
| 35 | 2206 | DRCGTST | SGLVW | FLSAIE | D99B00.R97B00. | S99B00.G99B00. | F99B00.L99B00. | 21 | 15 | 18 | 54 | No | No | No |
| | | | | | C99B00.G99B00. | L99B00.V99B00. | S99B00.A98B00. | | | | | | | |
| | | | | | T99B00.S98B00. | W99B00. | I99B00.E99B00. | | | | | | | |
| | | | | | T98B00. | | | | | | | | | |
| 18 | 1855 | ATIYIYA | CQSVR | IINCL | A99B00.T99B00. | C99B00.Q99B00. | I60F38.I99B00. | 21 | 15 | 14 | 50 | No | No | No |
| | | | | | I98B00.Y99B00. | S99B00.V99B00. | N99B00.C60D38. | | | | | | | |
| | | | | | I99B00.Y99B00. | R99B00. | L61D38.I61V38. | | | | | | | |
| | | | | | A99B00. | | | | | | | | | |
| 28 | 1090 | RP | AYGCV | PSEVFE | R99B00.P99B00. | A99A99.Y99B00. | P99P99.S99B00. | 6 | 15 | 18 | 39 | No | No | No |
| | | | | | | G99G99.C99B00. | E99B00.V99B00. | | | | | | | |
| | | | | | | V99B00. | F99B00.E98B00. | | | | | | | |
| 27 | 788 | MLTLSAR | LRAPT | FQSSNL | M97B00.L99B00. | L99L99.R99B00. | F99B00.Q99B00. | 21 | 15 | 18 | 54 | No | No | No |
| | | | | | T97B00.L99B00. | A99B00.P99P99. | S99B00.S99B00. | | | | | | | |
| | | | | | S99B00.A99B00. | T99B00. | N99B00.L98B00. | | | | | | | |
| | | | | | R99B00. | | | | | | | | | |
| 5 | 759 | HLVPVAT | ELSRW | QPSCYQ | H99B00.L99B00. | E99B00.L99L99. | Q99B00.P99B00. | 21 | 15 | 18 | 54 | No | No | No |
| | | | | | V97B00.P99B00. | S99B00.R99B00. | S99B00.C99B00. | | | | | | | |
| | | | | | V99B00.A99B00. | W99W99. | Y99B00.Q99B00. | | | | | | | |
| | | | | | T98B00. | | | | | | | | | |
| 19 | 743 | SVCYVYG | YSNLH | QINPML | S97B00.V99B00. | Y99B00.S99S99. | Q99B00.I99B00. | 21 | 15 | 18 | 54 | No | No | No |
| | | | | | C99B00.Y99B00. | N99N99.L99L99. | N99B00.P99B00. | | | | | | | |
| | | | | | V99B00.Y99B00. | H99B00. | M99B00.L99B00. | | | | | | | |
| | | | | | G99B00. | | | | | | | | | |
| 37 | 385 | NIGPCMC | GWSSS | FASVLK | N98B00.I99B00. | G99B00.W99W99. | F99B00.A99B00. | 21 | 15 | 18 | 54 | No | No | No |
| | | | | | G99B00.P99B00. | S99S99.S99S99. | S99B00.V99B00. | | | | | | | |
| | | | | | C99B00.M98B00. | S99S99. | L99B00.K98B00. | | | | | | | |
| | | | | | C99B00. | | | | | | | | | |
| 21 | 345 | RCVIVGC | GDGFD | NPFLGRFAGT | R99B00.C99B00. | G99G99.D99D99. | N99N99.P99B00. | 21 | 15 | 30 | 66 | No | No | No |
| | | | | | V99B00.I99B00. | G99B00.F99F99. | F99F99.L99B00. | | | | | | | |
| | | | | | V99V99.G99B00. | D99B00. | G99B00.R99R99. | | | | | | | |
| | | | | | C99B00. | | F99B00.A99B00. | | | | | | | |
| | | | | | | | G99B00.T99T99. | | | | | | | |
| 124 | 245 | ITVVVGS | CCGEP | PLIIQGCCY | I96B00.T99B00. | C99C99.C99C99. | P99P99.L99L99. | 21 | 15 | 27 | 63 | No | No | No |
| | | | | | V99B00.V99B00. | G99G99.E99E99. | I99I99.I99I99. | | | | | | | |
| | | | | | V99B00.G99B00. | P99B00. | Q99Q99.G99G99. | | | | | | | |
| | | | | | S99B00. | | C99C99.C99C99. | | | | | | | |
| | | | | | | | Y99B00. | | | | | | | |
| 48 | 229 | LALARVS | IGGCL | DTMPNYIYPM | L99B00.A98B00. | I99B00.G99B00. | D99B00.T99B00. | 21 | 15 | 30 | 66 | No | No | No |
| | | | | | L98B00.A99B00. | G99B00.C99B00. | M99B00.P99B00. | | | | | | | |
| | | | | | R99B00.V98B00. | L99B00. | N99B00.Y99B00. | | | | | | | |
| | | | | | S99B00. | | I99B00.Y99B00. | | | | | | | |
| | | | | | | | P99B00.M99B00. | | | | | | | |
| 13 | 173 | SSIYVYL | NNLNS | RINCDL | S97B00.S99B00. | N99N99.N99N99. | R99B00.I99B00. | 21 | 15 | 18 | 54 | No | No | No |
| | | | | | I98B00.Y98B00. | L99L99.N99N99. | N99B00.C99B00. | | | | | | | |
| | | | | | V99V99.Y99B00. | S99S99. | D99B00.L99B00. | | | | | | | |
| | | | | | L99B00. | | | | | | | | | |
| 139 | 134 | WMFVLSS | GNVPN | SGMAWIYPI | W99W99.M99B00. | G99G99.N99N99. | S98B00.G97B00. | 21 | 15 | 27 | 63 | No | No | No |
| | | | | | F99F99.V98B00. | V99V99.P99P99. | M98B00.A97B00. | | | | | | | |
| | | | | | L99L99.S99S99. | N99N99. | W97B00.I97B00. | | | | | | | |
| | | | | | S99S99. | | Y97B00.P98B00. | | | | | | | |
| | | | | | | | I98B00. | | | | | | | |
| 9 | 126 | HNVGVGT | MHAQS | NHTDHYIFPS | H98B00.N95B00. | M98B00.H98B00. | N99B00.H99B00. | 21 | 15 | 30 | 66 | No | No | No |
| | | | | | V98B00.G99B00. | A82B00.Q98B00. | T98B00.D99B00. | | | | | | | |
| | | | | | V98B00.G98B00. | S98B00. | H99B00.Y98B00. | | | | | | | |
| | | | | | T99B00. | | I99B00.F98B00. | | | | | | | |
| | | | | | | | P99B00.S99B00. | | | | | | | |
| 47 | 106 | PEISLIC | SYSLR | FASSCC | P99P99.E99E99. | S99S99.Y99Y99. | F99B00.A99B00. | 21 | 15 | 18 | 54 | No | No | No |
| | | | | | I97B00.S99S99. | S99S99.L99L99. | S99B00.S98B00. | | | | | | | |
| | | | | | L99L99.I99I99. | R99B00. | C99B00.C98B00. | | | | | | | |
| | | | | | C99C99. | | | | | | | | | |
| 357 | 80 | DTVYLLV | LLSSS | IINCTK | D99D99.T99T99. | L99L99.L99L99. | I99I99.I98B00. | 21 | 15 | 18 | 54 | No | No | No |
| | | | | | V99V99.Y98B00. | S99S99.S99S99. | N99N99.C99C99. | | | | | | | |
| | | | | | L99L99.L99L99. | S99S99. | T99T99.K99K99. | | | | | | | |
| | | | | | V98B00. | | | | | | | | | |
| 122 | 71 | ILVGIYW | VRDWS | QRSVFGSSG | I95B00.L99L99. | V99V99.R98B00. | Q99Q99.R98B00. | 21 | 15 | 27 | 63 | No | No | No |
| | | | | | V99V99.G99G99. | D99D99.W99W99. | S99S99.V99V99. | | | | | | | |
| | | | | | I99I99.Y99Y99. | S98B00. | F99F99.G99G99. | | | | | | | |
| | | | | | W99W99. | | S99S99.S99S99. | | | | | | | |
| | | | | | | | G99G99. | | | | | | | |
| 149 | 63 | ESQALRV | SVHCC | SYLGYIFPE | E99E99.S99S99. | S98B00.V98B00. | S99S99.Y99Y99. | 21 | 15 | 27 | 63 | No | No | No |
| | | | | | Q98B00.A99A99. | H99H99.C98B00. | L99L99.G99G99. | | | | | | | |
| | | | | | L99L99.R99R99. | C99C99. | Y99Y99.I99I99. | | | | | | | |
| | | | | | V99V99. | | F99F99.P99P99. | | | | | | | |
| | | | | | | | E99E99. | | | | | | | |
| 168 | 54 | CHSGALY | PMGVA | RMRTPCLMLSGIS | C99C99.H98B00. | P99P99.M99M99. | R99R99.M99M99. | 21 | 15 | 39 | 75 | No | No | No |
| | | | | | S99S99.G99G99. | G99G99.V99V99. | R99R99.T99T99. | | | | | | | |
| | | | | | A99A99.L99L99. | A99A99. | P99P99.C99C99. | | | | | | | |
| | | | | | Y96B00. | | L99L99.M99M99. | | | | | | | |
| | | | | | | | L99L99.S99S99. | | | | | | | |
| | | | | | | | G99G99.I99I99. | | | | | | | |
| | | | | | | | S81B00. | | | | | | | |
| 190 | 50 | VLMLL | IFQNR | YHGPG | V99V99.L99L99. | I99I99.F99F99. | Y99Y99.H99H99. | 21 | 15 | 15 | 51 | No | No | No |
| | | | | | K98B00.S99S99. | Q99Q99.N98B00. | G99G99.P99P99. | | | | | | | |
| | | | | | M99M99.L99L99. | R99R99. | G99G99. | | | | | | | |
| | | | | | L99L99. | | | | | | | | | |
| 232 | 48 | LLFT | RAAPS | PCTISG | L99L99.L99L99. | R99R99.A99A99. | P99P99.C99C99. | 12 | 15 | 18 | 45 | No | No | No |
| | | | | | F97B00.T99T99. | A99A99.P99P99. | T99T99.I99I99. | | | | | | | |
| | | | | | | S99S99. | S99S99.G99G99. | | | | | | | |
| 231 | 41 | YG | IITSG | SYLNSW | Y99Y99.G99G99. | I99I99.I99I99. | S99S99.Y99Y99. | 6 | 15 | 18 | 39 | No | No | No |
| | | | | | | T99T99.S99S99. | L99L99.N99N99. | | | | | | | |
| | | | | | | G99G99. | S99S99.W99W99. | | | | | | | |
| 417 | 38 | DMYGSLT | MFSDI | YGSTMTGCHC | D97B00.M99M99. | M99M99.F99F99. | Y99Y99.G99G99. | 21 | 15 | 30 | 66 | No | No | No |
| | | | | | Y99Y99.G99G99. | S99S99.D99D99. | S99S99.T99T99. | | | | | | | |
| | | | | | S99S99.L99L99. | I99I99. | M97B00.T99T99. | | | | | | | |
| | | | | | T99T99. | | G99G99.C99C99. | | | | | | | |
| | | | | | | | H99H99.C99C99. | | | | | | | |
| 36 | 27 | NMVYMKH | NWLH | ALLVRE | N96B00.M99M99. | N99N99.W99W99. | A51S44.L55B00. | 21 | 12 | 12 | 45 | No | No | No |
| | | | | | V99V99.Y99Y99. | L99L99.H99H99. | L74B00.V55B00. | | | | | | | |
| | | | | | M99M99.K99K99. | | R77B00.E51P40. | | | | | | | |
| | | | | | H99H99. | | | | | | | | | |
| 558 | 27 | PLATSG | FGD | YIQGAC | P99P99.L99L99. | F99F99.G99G99. | Y99Y99.I74B00. | 18 | 9 | 14 | 41 | No | No | No |
| | | | | | A99A99.T99T99. | D99D99. | Q74B00.G74B00. | | | | | | | |
| | | | | | S99S99.G99G99. | | A99A99.C74B00. | | | | | | | |
| 61 | 26 | VLSSEFT | VGAGW | TFSTRY | V99V99.L99L99. | V99V99.G99G99. | T99T99.F99F99. | 21 | 15 | 18 | 54 | No | No | No |
| | | | | | S99S99.S99S99. | A99A99.G99G99. | S99S99.T99T99. | | | | | | | |
| | | | | | E99E99.F99F99. | W99W99. | R99R99.Y99Y99. | | | | | | | |
| | | | | | T99T99. | | | | | | | | | |
| 621 | 26 | VHITRFV | VPGQR | SGLSFGAVR | V99V99.H99H99. | V99V99.P99P99. | S99S99.G99G99. | 21 | 15 | 27 | 63 | No | No | No |
| | | | | | I99I99.T99T99. | G99G99.Q99Q99. | L99L99.S99S99. | | | | | | | |
| | | | | | R99R99.F99F99. | R99R99. | F99F99.G99G99. | | | | | | | |
| | | | | | V99V99. | | A99A99.V99V99. | | | | | | | |
| | | | | | | | R99R99. | | | | | | | |
| 288 | 20 | RLRP | QYGRI | PDLVIYLMPN | R99R99.L99L99. | Q99Q99.Y99Y99. | P99P99.D99D99. | 12 | 15 | 30 | 57 | No | No | No |
| | | | | | R99R99.P99P99. | G99G99.R99R99. | L99L99.V99V99. | | | | | | | |
| | | | | | | I99I99. | I99I99.Y99Y99. | | | | | | | |
| | | | | | | | L99L99.M99M99. | | | | | | | |
| | | | | | | | P99P99.N99N99. | | | | | | | |
| 257 | 18 | AIGCNGL | CCFCS | NAHQYF | A99A99.I99I99. | C99C99.C99C99. | N99N99.A99A99. | 21 | 15 | 18 | 54 | No | No | No |
| | | | | | G99G99.C99C99. | F99F99.C99C99. | H99H99.Q99Q99. | | | | | | | |
| | | | | | N99N99.G99G99. | S99S99 | Y99Y99.F99F99. | | | | | | | |
| | | | | | L99L99. | | | | | | | | | |
| 401 | 16 | IDAHDSV | NIRRG | WITCES | I99I99.D99D99. | N99N99.I99I99. | W99W99.I99I99. | 21 | 15 | 18 | 54 | No | No | No |
| | | | | | A99A99.H99H99. | R99R99.R99R99. | T99T99.C99C99. | | | | | | | |
| | | | | | D99D99.S99S99. | G99G99. | E99E99.S99S99. | | | | | | | |
| | | | | | V99V99. | | | | | | | | | |
| 544 | 14 | VLICRTA | YAVRF | WPDCCN | V99V99.L99L99. | Y99Y99.A99A99. | W99W99.P99P99. | 21 | 15 | 18 | 54 | No | No | No |
| | | | | | I99I99.C99C99. | V99V99.R99R99. | D99D99.C99C99. | | | | | | | |
| | | | | | R99R99.T92B00. | F99F99. | C99C99.N99N99 | | | | | | | |
| | | | | | A99A99. | | | | | | | | | |
| 275 | 10 | VSGFRAC | DFCTG | FLSCDA | V99V99.S99S99. | D99D99.F99F99. | F99F99.L99L99. | 21 | 15 | 18 | 54 | No | No | No |
| | | | | | G99G99.F99F99. | C99C99.T99T99. | S99S99.C99C99. | | | | | | | |
| | | | | | R99R99.A99A99. | G99G99. | D99D99.A99A99 | | | | | | | |
| | | | | | C99C99. | | | | | | | | | |
| 515 | 10 | FDAAQAW | VYYVA | KDM | F99F99.D99D99. | V99V99.Y99Y99. | K99K99.D99D99. | 21 | 15 | 9 | 45 | No | No | No |
| | | | | | A99A99.A99A99. | Y99Y99.V99V99. | M99M99. | | | | | | | |
| | | | | | Q99Q99.A90B00. | A99A99. | | | | | | | | |
| | | | | | W99W99. | | | | | | | | | |
| 488 | 8 | NCNRVIS | RSPQV | WPDCIV | N99N99.C99C99. | R99R99.S99S99. | W99W99.P99P99. | 21 | 15 | 18 | 54 | No | No | No |
| | | | | | N99N99.R99R99. | P99P99.Q99Q99. | D99D99.C99C99. | | | | | | | |
| | | | | | V99V99.I99I99. | V99V99. | I99I99.V99V99. | | | | | | | |
| | | | | | S99S99. | | | | | | | | | |
| 518 | 8 | CVINYHV | TPWVM | MCCAATFWDP | C87B00.V87B00. | T99T99.P99P99. | M99M99.C99C99. | 21 | 15 | 30 | 66 | No | No | No |
| | | | | | I87B00.N87B00. | W99W99.V99V99. | C99C99.A99A99. | | | | | | | |
| | | | | | Y87B00.H87B00. | M99M99. | A99A99.T99T99. | | | | | | | |
| | | | | | V87B00. | | F99F99.W99W99. | | | | | | | |
| | | | | | | | D99D99.P99P99. | | | | | | | |
| 531 | 7 | VCYTGWD | ANQPR | GPDILSFYGC | V99V99.C99C99. | A99A99.N99N99. | G99G99.P99P99. | 21 | 15 | 30 | 66 | No | No | No |
| | | | | | Y99Y99.T99T99. | Q99Q99.P99P99. | D99D99.I99I99. | | | | | | | |
| | | | | | G99G99.W99W99. | R99R99. | L99L99.S99S99. | | | | | | | |
| | | | | | D99D99. | | F99F99.Y99Y99. | | | | | | | |
| | | | | | | | G99G99.C99C99. | | | | | | | |
| 595 | 6 | TRTELRS | LIFIM | TDDFFN | T99T99.R99R99. | L99L99.I99I99. | T83B00.D83B00. | 21 | 15 | 18 | 54 | No | No | No |
| | | | | | T99T99.E99E99. | F99F99.I99I99. | D83B00.F99F99. | | | | | | | |
| | | | | | L99L99.R99R99. | M99M99. | F83B00.N83B00. | | | | | | | |
| | | | | | S99S99. | | | | | | | | | |
| 705 | 6 | SDTVGLV | YFRHS | ITF | S99S99.D99D99. | Y99Y99.F99F99. | I99I99.T99T99. | 21 | 15 | 9 | 45 | No | No | No |
| | | | | | T99T99.V99V99. | R99R99.H99H99. | F99F99. | | | | | | | |
| | | | | | G99G99.L99L99. | S99S99. | | | | | | | | |
| | | | | | V99V99. | | | | | | | | | |

TABLE 8
List of EGFP binder clusters. A list containing key information for all predicted EGFP binding clusters. Cluster ID, size, CDR representative sequences, CDR consensus sequences, CDR scores
(see Materials and Methods), and whether each CDR is unique to EGFP and not found in RBD clusters were shown.
Column 3-CDR1_rep-SEQ ID NOS: 5461-5577
Column 4-CDR2_rep: SEQ ID NOS: 5578-5698
Column 5-CDR3_rep: SEQ ID NOS: 5699-5810

| | | | | | | | | | CDR1_ | CDR2_ | CDR3_ | total_ | CDR1_ | CDR2_ | CDR3_ |
|---|---|---|---|---|---|---|---|---|---|---|---|---|---|---|---|
| cluster_type | cluster_ID | cluster_size | CDR1_rep | CDR2_rep | CDR3_rep | CDR1_consensus | CDR2_consensus | CDR3_consensus | score | score | score | score | unique | unique | unique |
| RC | 6 | 5182 | LNL | EVS | HMK | L99B00.N97B00. | E99B00.V99B00. | H99B00.M99B00. | 21 | 15 | 27 | 63 | Yes | Yes | Yes |
| | | | AYF | AK | LWS | L99B00.A99B00. | S99B00.A99B00. | K99B00.L99B00. | | | | | | | |
| | | | V | | NSM | Y99B00.F99B00. | K99B00. | W99B00.S99B00. | | | | | | | |
| | | | | | | V99B00. | | N99B00.S99B00. | | | | | | | |
| | | | | | | | | M98B00. | | | | | | | |
| RC | 5 | 1189 | VC | DFL | SHS | V99B00.C99B00. | D99B00.F99F99. | S99B00.H99B00. | 6 | 15 | 39 | 60 | Yes | Yes | Yes |
| | | | | AA | TDL | | L99L99.A99B00. | S99S99.T99B00. | | | | | | | |
| | | | | | YWR | | A99B00. | D99B00.L99L99. | | | | | | | |
| | | | | | GHP | | | Y99Y99.W99B00. | | | | | | | |
| | | | | | G | | | R99B00.G99B00. | | | | | | | |
| | | | | | | | | H99B00.P99B00. | | | | | | | |
| | | | | | | | | G99B00. | | | | | | | |
| RC | 20 | 463 | DISA | FIFA | TTC | D99D99.I99I99. | F99B00.I99B00. | T99T99.T93B00. | 21 | 15 | 30 | 66 | Yes | Yes | Yes |
| | | | YHR | R | KICY | S99S99.A99B00. | F99B00.A99B00. | C93B00K93B00. | | | | | | | |
| | | | | | GYQ | Y99Y99.H98B00. | R99R99. | I93B00.C93B00. | | | | | | | |
| | | | | | | R99R99. | | Y93B00.G93B00. | | | | | | | |
| | | | | | | | | Y93B00.Q93B00. | | | | | | | |
| RC | 31 | 257 | SCSI | STW | TTL | S99B00.C99B00. | S99S99.T99T99. | T99T99.T98B00. | 21 | 15 | 18 | 54 | Yes | Yes | Yes |
| | | | SNS | DR | YFR | S98B00.I99B00. | W99W99.D99D99. | L99B00.Y99B00. | | | | | | | |
| | | | | | | S99B00.N96B00 | R99R99. | F99F99.R99B00. | | | | | | | |
| | | | | | | S98B00. | | | | | | | | | |
| RC | 22 | 244 | GKV | LYY | NHS | G97B00.K97B00. | L99B00.Y99Y99. | N99B00.H99B00. | 21 | 15 | 18 | 54 | Yes | Yes | Yes |
| | | | GVC | GL | MMF | V99V99.G99B00. | Y99B00.G99B00. | S99B00.M99B00. | | | | | | | |
| | | | S | | | V99V99.C99B00. | L99L99. | M99B00.F99B00. | | | | | | | |
| | | | | | | S99B00. | | | | | | | | | |
| RC | 53 | 141 | KM | LLL | SRY | K98B00.M99M99. | L99L99.L99B00. | S99B00.R99R99. | 21 | 15 | 18 | 54 | Yes | Yes | Yes |
| | | | WR | GT | VSY | W99W99.R99B00. | L99L99.G99G99. | Y99Y99.V99V99. | | | | | | | |
| | | | GLD | | | G99G99.L99L99. | T99T99. | S99S99.Y99B00. | | | | | | | |
| | | | | | | D99B00. | | | | | | | | | |
| RC | 39 | 113 | PMP | NTW | TDL | P99P99.M99M99. | N99N99.T99T99. | T99B00.D99B00. | 21 | 15 | 18 | 54 | Yes | Yes | Yes |
| | | | QVG | AR | RFV | P99P99.Q99B00. | W99W99.A99A99. | L99B00.R98B00. | | | | | | | |
| | | | | | | V99V99.G99G99. | R99R99. | F99F99.V99B00. | | | | | | | |
| | | | | | | V99V99. | | | | | | | | | |
| RC | 7 | 69 | DCC | EWF | TDL | D99D99.C98B00. | E99E99.W99W99. | T99T99.D98B00. | 21 | 15 | 18 | 54 | Yes | Yes | Yes |
| | | | FLG | GR | RFK | C99C99.F99F99. | F99F99.G99G99. | L98B00.R98B00. | | | | | | | |
| | | | K | | | L99L99.G98B00. | R99R99. | F98B00.K98B00. | | | | | | | |
| | | | | | | K98B00. | | | | | | | | | |
| RC | 56 | 57 | YPF | SFD | S | Y99Y99.P99P99. | S99S99.F98B00. | S98B00. | 21 | 15 | 3 | 39 | Yes | Yes | Yes |
| | | | MPF | EA | | F99F99.M99M99. | D99D99.E99E99. | | | | | | | | |
| | | | F | | | P99P99.F98B00. | A99A99. | | | | | | | | |
| | | | | | | F99F99. | | | | | | | | | |
| RC | 27 | 41 | AGC | WVF | SNC | A99A99.G99G99. | W99W99.V99V99. | S92B00.N92B00. | 21 | 15 | 18 | 54 | Yes | Yes | Yes |
| | | | GFM | GS | VLL | C99C99.G95B00. | F99F99.G99G99. | C92B00.V92B00. | | | | | | | |
| | | | R | | | F99F99.M99M99. | S99S99. | L92B00.L92B00. | | | | | | | |
| | | | | | | R99R99. | | | | | | | | | |
| RC | 43 | 37 | PDV | RTF | TNC | P97B00.D94B00. | R97B00.T99T99. | T99T99.N99N99. | 21 | 15 | 18 | 54 | Yes | Yes | Yes |
| | | | THC | NR | YFV | V97B00.T97B00. | F99F99.N99N99. | C97B00.Y97B00. | | | | | | | |
| | | | T | | | H94B00.C97B00. | R99R99. | F99F99.V97B00. | | | | | | | |
| | | | | | | T97B00. | | | | | | | | | |
| RC | 61 | 28 | LKV | RMG | CAT | L99L99.K99K99. | R99R99.M99M99. | C99C99.A99A99. | 12 | 15 | 30 | 57 | Yes | Yes | Yes |
| | | | G | VL | FST | V99V99.G99G99. | G99G99.V99V99. | T99T99.F99F99. | | | | | | | |
| | | | | | CVW | | L99L99. | S99S99.T99T99. | | | | | | | |
| | | | | | L | | | C99C99.V99V99. | | | | | | | |
| | | | | | | | | W99W99.L99L99. | | | | | | | |
| RC | 66 | 26 | TPF | FTW | TSQ | T96B00.P99P99. | F99F99.T99T99. | T99T99.S99S99. | 21 | 15 | 18 | 54 | Yes | Yes | Yes |
| | | | NSY | YR | WFC | F99F99.N99N99. | W99W99.Y99Y99. | Q99Q99.W99W99. | | | | | | | |
| | | | S | | | S99S99.Y99Y99. | R99R99. | F99F99.C99C99. | | | | | | | |
| | | | | | | S99S99. | | | | | | | | | |
| RC | 8 | 26 | AGA | SLG | F | A99A99.G99G99. | S99S99.L99L99. | F99F99. | 21 | 15 | 3 | 39 | Yes | Yes | Yes |
| | | | LRR | HC | | A99A99.L99L99. | G99G99.H99H99. | | | | | | | | |
| | | | S | | | R99R99.R99R99. | C99C99. | | | | | | | | |
| | | | | | | S99S99. | | | | | | | | | |
| RC | 73 | 25 | LYAI | RMG | TTIY | L99L99.Y99Y99. | R99R99.M99M99. | T99T99.T99T99. | 21 | 15 | 30 | 66 | Yes | Yes | Yes |
| | | | ERH | LL | GSC | A99A99.I99I99. | G99G99.L99L99. | I99I99.Y99Y99. | | | | | | | |
| | | | | | FCQ | E99E99.R99R99. | L99L99. | G99G99.S99S99. | | | | | | | |
| | | | | | | H99H99. | | C99C99.F99F99. | | | | | | | |
| | | | | | | | | C99C99.Q99Q99. | | | | | | | |
| RC | 51 | 24 | MW | WW | IDTA | M99M99.W99W99. | W99W99.W99W99. | I58B00.D54B00. | 21 | 15 | 14 | 50 | Yes | Yes | Yes |
| | | | HAF | AAS | TGAI | H99H99.A99A99. | A99A99.A99A99. | T45B00.A45B00. | | | | | | | |
| | | | DT | | FR | F99F99.D99D99. | S99S99. | T45B00.G45B00. | | | | | | | |
| | | | | | | T99T99. | | A45B00.I45B00. | | | | | | | |
| | | | | | | | | F54B00.R54B00. | | | | | | | |
| RC | 67 | 24 | WPL | WFS | FRR | W95B00.P95B00. | W99W99.F99F99. | F99F99.R99R99. | 21 | 15 | 39 | 75 | Yes | Yes | Yes |
| | | | GFY | HD | KYQ | L99L99.G95B00. | S99S99.H99H99. | R99R99.K99K99. | | | | | | | |
| | | | T | | EFG | F95B00.Y95B00. | D99D99. | Y99Y99.Q99Q99. | | | | | | | |
| | | | | | AGS | T91B00. | | E99E99.F99F99. | | | | | | | |
| | | | | | V | | | G99G99.A99A99. | | | | | | | |
| | | | | | | | | G99G99.S99S99. | | | | | | | |
| | | | | | | | | V99V99. | | | | | | | |
| RC | 68 | 22 | CTS | SDS | S | C99C99.T99T99. | S99S99.D99D99. | S99S99. | 21 | 15 | 3 | 39 | Yes | Yes | Yes |
| | | | NIR | AY | | S99S99N99N99. | S99S99.A99A99. | | | | | | | | |
| | | | P | | | I99I99.R99R99. | Y99Y99. | | | | | | | | |
| | | | | | | P99P99. | | | | | | | | | |
| RC | 88 | 22 | WT | CLG | RPL | W99W99.T99T99. | C99C99.L99L99. | R99R99.P99P99. | 6 | 15 | 24 | 45 | Yes | Yes | Yes |
| | | | | HR | SPN | | G99G99.H99H99. | L99L99.S99S99. | | | | | | | |
| | | | | | LH | | R99R99. | P99P99.N99N99. | | | | | | | |
| | | | | | | | | L99L99.H99H99. | | | | | | | |
| RC | 58 | 20 | LAT | EYW | SSD | L99L99.A99A99. | E99E99.Y99Y99. | S99S99.S99S99. | 33 | 15 | 18 | 66 | Yes | Yes | Yes |
| | | | VCC | GR | KFV | T99T99.V99V99. | W99W99.G99G99. | D99D99.K99K99. | | | | | | | |
| | | | GGL | | | C99C99.C99C99. | R99R99. | F99F99.V99V99. | | | | | | | |
| | | | WA | | | G95B00.G99G99. | | | | | | | | | |
| | | | | | | L99L99.W99W99. | | | | | | | | | |
| | | | | | | A99A99. | | | | | | | | | |
| RC | 85 | 20 | PFW | CPF | NNE | P99P99.F99F99. | C99C99.P99P99. | N99N99.N99N99. | 21 | 15 | 18 | 54 | Yes | Yes | Yes |
| | | | TLD | HR | YM | W99W99.T99T99. | F99F99.H99H99. | E99E99.Y99Y99. | | | | | | | |
| | | | K | | W | L95B00.D99D99. | R99R99. | M99M99.W99W99. | | | | | | | |
| | | | | | | K99K99. | | | | | | | | | |
| RC | 82 | 19 | VYV | VAN | RRY | V99V99.Y99Y99. | V99V99.A99A99. | R99R99.R99R99. | 21 | 15 | 27 | 63 | Yes | Yes | Yes |
| | | | GW | DA | CRY | V99V99.G99G99. | N99N99.D99D99. | Y99Y99.C99C99. | | | | | | | |
| | | | HD | | AVV | W99W99.H99H99. | A99A99. | R99R99.Y99Y99. | | | | | | | |
| | | | | | | D99D99. | | A99A99.V99V99. | | | | | | | |
| | | | | | | | | V99V99. | | | | | | | |
| RC | 62 | 16 | DLY | VVT | RFR | D99D99.L99L99. | V99V99.V99V99. | R99R99.F99F99. | 21 | 15 | 39 | 75 | Yes | Yes | Yes |
| | | | LLR | CR | SHN | Y99Y99.L99L99. | T99T99.C99C99. | R93B00.S99S99. | | | | | | | |
| | | | N | | VYC | L99L99.R99R99. | R99R99. | H99H99.N99N99. | | | | | | | |
| | | | | | MDL | N99N99. | | V99V99.Y99Y99. | | | | | | | |
| | | | | | V | | | C99C99.M99M99. | | | | | | | |
| | | | | | | | | D99D99.L99L99. | | | | | | | |
| | | | | | | | | V99V99. | | | | | | | |
| RC | 65 | 16 | LTC | TVF | SDQ | L99L99.T99T99. | T99T99.V99V99. | S93B00.D93B00. | 21 | 15 | 18 | 54 | Yes | Yes | Yes |
| | | | GSD | NV | FMT | C99C99.G99G99. | F99F99N99N99. | Q93B00.F93B00. | | | | | | | |
| | | | R | | | S99S99.D99D99. | V99V99. | M93B00.T93B00 | | | | | | | |
| | | | | | | R99R99. | | | | | | | | | |
| RC | 105 | 14 | YW | WW | TKS | Y99Y99.W99W99. | W99W99.W99W99. | B00 | 21 | 15 | 0 | 36 | Yes | Yes | Yes |
| | | | GVF | HDS | SAL | G99G99.V99V99. | H99H99.D92B00. | | | | | | | | |
| | | | MD | | | F99F99.M99M99. | S99S99. | | | | | | | | |
| | | | | | | D92B00. | | | | | | | | | |
| RC | 41 | 14 | SVL | RTW | TTC | S99S99.V99V99. | R99R99.T99T99. | T99T99.T99T99. | 21 | 15 | 18 | 54 | Yes | Yes | Yes |
| | | | SVT | VR | TFE | L99L99.S99S99. | W99W99.V99V99. | C99C99.T99T99. | | | | | | | |
| | | | A | | | V99V99.T99T99. | R99R99. | F99F99.E99E99. | | | | | | | |
| | | | | | | A99A99. | | | | | | | | | |
| RC | 102 | 13 | LYIA | RCA | ITVV | L99L99.Y99Y99. | R99R99.C99C99. | I99I99.T99T99. | 21 | 15 | 18 | 54 | Yes | Yes | Yes |
| | | | RSR | RY | IA | I99I99.A99A99. | A99A99.R99R99. | V99V99.V99V99. | | | | | | | |
| | | | | | | R99R99.S99S99. | Y99Y99. | I99I99.A99A99. | | | | | | | |
| | | | | | | R99R99. | | | | | | | | | |
| RC | 59 | 13 | MRV | SSL | SNL | M99M99.R92B00. | S99S99.S99S99. | S99S99.N99N99. | 12 | 15 | 39 | 66 | Yes | Yes | Yes |
| | | | N | SS | QLC | V99V99.N99N99. | L99L99.S99S99. | L99L99.Q99Q99. | | | | | | | |
| | | | | | LTW | | S99S99. | L99L99.C99C99. | | | | | | | |
| | | | | | LDI | | | L99L99.T99T99. | | | | | | | |
| | | | | | M | | | W99W99.L99L99. | | | | | | | |
| | | | | | | | | D99D99.I99I99. | | | | | | | |
| | | | | | | | | M99M99. | | | | | | | |
| RC | 106 | 11 | VCS | STA | FM | V99V99.C99C99. | S99S99.T99T99. | F99F99.M99M99. | 21 | 15 | 6 | 42 | Yes | Yes | Yes |
| | | | GVR | VY | | S99S99.G99G99. | A99A99.V99V99. | | | | | | | | |
| | | | G | | | V99V99.R99R99. | Y99Y99. | | | | | | | | |
| | | | | | | G99G99. | | | | | | | | | |
| RC | 37 | 11 | GHS | FVF | TSE | G99G99.H90B00. | F99F99.V99V99. | T99T99.S99S99. | 21 | 15 | 18 | 54 | Yes | Yes | Yes |
| | | | PCW | TG | LMV | S99S99.P99P99. | F99F99.T99T99. | E99E99.L99L99. | | | | | | | |
| | | | S | | | C99C99.W99W99. | G99G99. | M99M99.V99V99. | | | | | | | |
| | | | | | | S99S99. | | | | | | | | | |
| RC | 9 | 11 | TTL | ATC | LAP | T90B00.T99T99. | A99A99.T99T99. | L99L99.A99A99. | 21 | 15 | 30 | 66 | Yes | Yes | Yes |
| | | | PGL | WG | CSM | L99L99.P99P99. | C99C99.W99W99. | P99P99.C99C99. | | | | | | | |
| | | | V | | GW | G99G99.L99L99. | G99G99. | S99S99.M99M99. | | | | | | | |
| | | | | | VM | V99V99. | | G99G99.W99W99. | | | | | | | |
| | | | | | | | | V90B00.M99M99. | | | | | | | |
| RC | 29 | 10 | LYG | SSW | GI | L99L99.Y90B00. | S99S99.S99S99. | G99G99.I99I99. | 21 | 12 | 6 | 39 | Yes | Yes | Yes |
| | | | PRR | R | | G99G99.P99P99. | W99W99.R99R99. | | | | | | | | |
| | | | W | | | R99R99.R99R99. | | | | | | | | | |
| | | | | | | W99W99. | | | | | | | | | |
| RC | 72 | 10 | VPV | FLW | TSD | V99V99.P90B00. | F99F99.L99L99. | T99T99.S99S99. | 21 | 15 | 18 | 54 | Yes | Yes | Yes |
| | | | QLW | MR | RFA | V99V99.Q99Q99. | W99W99.M90B00. | D99D99.R99R99. | | | | | | | |
| | | | S | | | L99L99.W99W99. | R99R99. | F99F99.A99A99. | | | | | | | |
| | | | | | | S90B00. | | | | | | | | | |
| RC | 75 | 10 | LYQ | WFT | DVH | L99L99.Y99Y99. | W99W99.F99F99. | D99D99.V99V99. | 21 | 15 | 30 | 66 | Yes | Yes | Yes |
| | | | RIL | GR | GYY | Q99Q99.R99R99. | T99T99.G99G99. | H99H99.G99G99. | | | | | | | |
| | | | M | | VFP | I99I99.L99L99. | R99R99. | Y99Y99.Y99Y99. | | | | | | | |
| | | | | | N | M90B00. | | V99V99.F99F99. | | | | | | | |
| | | | | | | | | P99P99.N99N99. | | | | | | | |
| RC | 100 | 9 | LSH | TWF | TSL | L99L99.S99S99. | T99T99.W99W99. | T99T99.S99S99. | 21 | 15 | 18 | 54 | Yes | Yes | Yes |
| | | | SVF | HR | EMC | H99H99.S99S99. | F99F99.H99H99. | L99L99.E99E99. | | | | | | | |
| | | | A | | | V99V99.F99F99. | R99R99. | M99M99.C99C99. | | | | | | | |
| | | | | | | A99A99. | | | | | | | | | |
| RC | 107 | 9 | SGV | WYD | VYV | S99S99.G99G99. | W99W99.Y99Y99. | V99V99.Y99Y99. | 21 | 15 | 39 | 75 | Yes | Yes | Yes |
| | | | GCF | | PSH | V99V99.G99G99. | D99D99.L99L99. | V99V99.P99P99. | | | | | | | |
| | | | D | | FNA | C99C99.F99F99. | G99G99. | S99S99.H99H99. | | | | | | | |
| | | | | | W | D99D99. | | F99F99.N99N99. | | | | | | | |
| | | | | | WK | | | A99A99.W99W99. | | | | | | | |
| | | | | | | | | S99S99.W99W99. | | | | | | | |
| | | | | | | | | K99K99. | | | | | | | |
| RC | 19 | 9 | LIGS | TLS | GVL | L99L99.I99I99. | T99T99.L99L99. | G99G99.V99V99. | 21 | 15 | 12 | 48 | Yes | Yes | Yes |
| | | | RHC | ST | T | G99G99.S99S99. | S99S99.S99S99. | L99L99.T99T99. | | | | | | | |
| | | | | | | R99R99.H99H99. | T99T99. | | | | | | | | |
| | | | | | | C99C99. | | | | | | | | | |
| RC | 69 | 9 | IGM | TRA | DSV | I99I99.G99G99. | T99T99.R99R99. | D99D99.S99S99. | 21 | 15 | 18 | 54 | Yes | Yes | Yes |
| | | | CRY | TW | LWL | M99M99.C99C99. | A99A99.T99T99. | V99V99.L99L99. | | | | | | | |
| | | | Y | | | R99R99.Y99Y99. | W99W99. | W99W99.L99L99. | | | | | | | |
| | | | | | | Y99Y99. | | | | | | | | | |
| RC | 74 | 9 | GMN | FTV | HTF | G99G99.M99M99. | F99F99.T99T99. | H99H99.T99T99. | 21 | 15 | 39 | 75 | Yes | Yes | Yes |
| | | | LSR | ED | RAF | N99N99.L99L99. | V99V99.E99E99. | F99F99R99R99. | | | | | | | |
| | | | H | | GS | S99S99.R88B00. | D99D99. | A99A99.F99F99. | | | | | | | |
| | | | | | WVS | H99H99. | | G99G99.S99S99. | | | | | | | |
| | | | | | AS | | | W99W99.V99V99. | | | | | | | |
| | | | | | | | | S99S99.A99A99. | | | | | | | |
| | | | | | | | | S99S99. | | | | | | | |
| RC | 76 | 9 | CYT | WW | ASN | C99C99.Y88B00. | W99W99.W99W99. | A99A99.S99S99. | 21 | 15 | 30 | 66 | Yes | Yes | Yes |
| | | | AFR | HLT | DYD | T99T99.A99A99. | H99H99.L99L99. | N99N99.D99D99. | | | | | | | |
| | | | V | | AISI | F99F99.R99R99. | T99T99. | Y99Y99.D99D99. | | | | | | | |
| | | | | | | V99V99. | | A99A99.I99I99. | | | | | | | |
| | | | | | | | | S99S99.I99I99. | | | | | | | |
| RC | 93 | 9 | QYS | PRG | FNS | Q99Q99.Y99Y99. | P99P99.R99R99. | F99F99.N99N99. | 21 | 15 | 18 | 54 | Yes | Yes | Yes |
| | | | VHT | HW | CVE | S99S99.V99V99. | G99G99.H99H99. | S99S99.C99C99. | | | | | | | |
| | | | T | | | H99H99.T99T99. | W99W99. | V99V99.E99E99. | | | | | | | |
| | | | | | | T99T99. | | | | | | | | | |
| RC | 121 | 8 | ALC | ICH | YTM | A99A99.L99L99. | I99I99.C99C99. | Y99Y99.T99T99. | 21 | 15 | 18 | 54 | Yes | Yes | Yes |
| | | | VIS | DR | RFY | C99C99.V99V99. | H99H99.D99D99. | M99M99.R99R99. | | | | | | | |
| | | | G | | | I99I99.S87B00. | R99R99. | F99F99.Y99Y99. | | | | | | | |
| | | | | | | G99G99. | | | | | | | | | |
| RC | 18 | 8 | TRL | SMC | GV | T99T99.R99R99. | S99S99.M99M99. | G99G99.V99V99. | 21 | 15 | 6 | 42 | Yes | Yes | Yes |
| | | | PSL | RV | | L99L99.P99P99. | C99C99.R99R99. | | | | | | | | |
| | | | T | | | S99S99.L99L99. | V99V99. | | | | | | | | |
| | | | | | | T99T99. | | | | | | | | | |
| RC | 47 | 8 | YYA | PMG | LTH | Y99Y99.Y99Y99. | P99P99.M99M99. | L99L99.T99T99. | 21 | 15 | 27 | 63 | Yes | Yes | Yes |
| | | | CGS | VL | PFG | A99A99.C99C99. | G99G99.V99V99. | H99H99.P99P99. | | | | | | | |
| | | | A | | EDV | G99G99.S99S99. | L99L99. | F99F99.G99G99. | | | | | | | |
| | | | | | | A99A99. | | E99E99.D99D99. | | | | | | | |
| | | | | | | | | V99V99. | | | | | | | |
| RC | 63 | 8 | NFR | SVF | LVG | N99N99.F99F99. | S99S99.V99V99. | L99L99.V99V99. | 21 | 15 | 30 | 66 | Yes | Yes | Yes |
| | | | SFF | SG | SLT | R99R99.S99S99. | F99F99.S99S99. | G99G99.S99S99. | | | | | | | |
| | | | L | | GGL | F99F99.F99F99. | G99G99. | L99L99.T99T99. | | | | | | | |
| | | | | | N | L99L99. | | G99G99.G99G99. | | | | | | | |
| | | | | | | | | L99L99.N99N99. | | | | | | | |
| RC | 89 | 8 | GFD | SLC | LPA | G99G99.F99F99. | S99S99.L99L99. | L99L99.P99P99. | 20 | 15 | 27 | 62 | Yes | Yes | Yes |
| | | | TGF | CS | PIK | D99D99.T99T99. | C99C99.C87B00. | A99A99.P99P99. | | | | | | | |
| | | | L | | MVD | G75B00.F99F99. | S99S99. | I99I99.K99K99. | | | | | | | |
| | | | | | | L99L99. | | M99M99.V99V99. | | | | | | | |
| | | | | | | | | D99D99. | | | | | | | |
| RC | 101 | 7 | GSV | CLR | IPF | G99G99.S99S99. | C99C99.L99L99. | I99I99.P99P99. | 21 | 15 | 30 | 66 | Yes | Yes | Yes |
| | | | RIS | SA | WPF | V99V99.R99R99. | R99R99.S99S99. | F99F99.W99W99. | | | | | | | |
| | | | N | | FST | I99I99.S99S99. | A99A99. | P99P99.F99F99. | | | | | | | |
| | | | | | C | N99N99. | | F99F99.S99S99. | | | | | | | |
| | | | | | | | | T99T99.C99C99. | | | | | | | |
| RC | 122 | 7 | AND | VVF | TNL | A99A99.N99N99. | V99V99.V99V99. | T99T99.N99N99. | 21 | 15 | 18 | 54 | Yes | Yes | Yes |
| | | | RLT | TV | YMS | D99D99.R99R99. | F99F99.T99T99. | L99L99.Y99Y99. | | | | | | | |
| | | | S | | | L99L99.T99T99. | V99V99. | M99M99.S99S99. | | | | | | | |
| | | | | | | S99S99. | | | | | | | | | |
| RC | 15 | 7 | GLR | SLF | GDD | G99G99.L99L99. | S99S99.L99L99. | G99G99.D99D99. | 21 | 15 | 18 | 54 | Yes | Yes | Yes |
| | | | GVR | AQ | YFR | R99R99.G99G99. | F99F99.A99A99. | D99D99.Y99Y99. | | | | | | | |
| | | | S | | | V99V99.R99R99. | Q99Q99. | F99F99.R99R99 | | | | | | | |
| | | | | | | S99S99. | | | | | | | | | |
| RC | 64 | 7 | NPR | LRY | YTC | N99N99.P99P99. | L99L99.R99R99. | Y99Y99.T99T99. | 21 | 15 | 18 | 54 | Yes | Yes | Yes |
| | | | PIGI | GG | TGY | R99R99.P99P99. | Y99Y99.G99G99. | C99C99.T99T99. | | | | | | | |
| | | | | | | I99I99.G99G99. | G99G99. | G99G99.Y99Y99. | | | | | | | |
| | | | | | | I99I99. | | | | | | | | | |
| RC | 91 | 7 | CSE | CVF | CTL | C99C99.S99S99. | C99C99.V99V99. | C99C99.T99T99. | 21 | 15 | 18 | 54 | Yes | Yes | Yes |
| | | | GYT | AV | RMT | E99E99.G99G99. | F99F99.A99A99. | L99L99.R99R99. | | | | | | | |
| | | | L | | | Y99Y99.T99T99. | V99V99. | M99M99.T99T99. | | | | | | | |
| | | | | | | L99L99. | | | | | | | | | |
| RC | 98 | 7 | SMD | KFG | GTS | S99S99.M99M99. | K99K99.F99F99. | G99G99.T99T99. | 21 | 15 | 30 | 66 | Yes | Yes | Yes |
| | | | RRF | VL | APW | D99D99.R99R99. | G99G99.V99V99. | S99S99.A85B00. | | | | | | | |
| | | | T | | SLL | R99R99.F99F99. | L99L99. | P99P99.W99W99. | | | | | | | |
| | | | | | V | T99T99. | | S99S99.L85B00. | | | | | | | |
| | | | | | | | | L99L99.V99V99. | | | | | | | |
| RC | 84 | 6 | FPM | RTW | TTIE | F99F99.P99P99. | R99R99.T99T99. | T99T99.T99T99. | 21 | 15 | 18 | 54 | Yes | Yes | Yes |
| | | | DAG | RR | FC | M99M99.D99D99. | W99W99.R99R99. | I99I99.E99E99. | | | | | | | |
| | | | C | | | A99A99.G99G99. | R99R99. | F99F99.C99C99. | | | | | | | |
| | | | | | | C99C99. | | | | | | | | | |
| RC | 97 | 6 | SLV | VVF | TNL | S83B00.L99L99. | V83B00.V83B00. | T99T99.N66B00. | 21 | 15 | 14 | 50 | Yes | Yes | Yes |
| | | | NFH | SR | CCN | V83B00.N83B00. | F99F99.S83B00. | L66B00.C66B00. | | | | | | | |
| | | | R | | | F83B00.H99H99. | R99R99. | C66B00.N99N99. | | | | | | | |
| | | | | | | R83B00. | | | | | | | | | |
| RC | 108 | 5 | CAS | LTW | TTE | C99C99.A99A99. | L99L99.T99T99. | T99T99.T99T99. | 21 | 15 | 39 | 75 | Yes | Yes | Yes |
| | | | AFH | TV | PCL | S99S99.A99A99. | W99W99.T99T99. | E99E99.P99P99. | | | | | | | |
| | | | E | | EIG | F99F99.H99H99. | V99V99. | C99C99.L99L99. | | | | | | | |
| | | | | | SGF | E99E99. | | E99E99.I99I99. | | | | | | | |
| | | | | | L | | | G99G99.S99S99. | | | | | | | |
| | | | | | | | | G99G99.F99F99. | | | | | | | |
| | | | | | | | | L99L99. | | | | | | | |
| RC | 110 | 5 | RSN | TFG | ELA | R99R99.S99S99. | T99T99.F99F99. | E99E99.L99L99. | 15 | 15 | 38 | 68 | Yes | Yes | Yes |
| | | | SW | LL | THT | N99N99.S99S99. | G99G99.L99L99. | A99A99.T99T99. | | | | | | | |
| | | | | | VDL | W99W99. | L99L99. | H80B00.T99T99. | | | | | | | |
| | | | | | FGF | | | V99V99.D99D99. | | | | | | | |
| | | | | | G | | | L99L99.F99F99. | | | | | | | |
| | | | | | | | | G99G99.F99F99. | | | | | | | |
| | | | | | | | | G99G99. | | | | | | | |
| RC | 111 | 5 | MRS | SFA | QYG | M99M99.R60G40. | S99S99.F99F99. | Q99Q99.Y99Y99. | 20 | 15 | 18 | 53 | Yes | Yes | Yes |
| | | | TRK | AW | PVM | S99S99.T99T99. | A99A99.A99A99. | G99G99.P99P99. | | | | | | | |
| | | | T | | | R99R99.K99K99. | W99W99. | V99V99.M99M99. | | | | | | | |
| | | | | | | T99T99. | | | | | | | | | |
| RC | 115 | 5 | CLG | WW | TNP | C99C99.L99L99. | W99W99.W99W99. | T99T99.N99N99. | 15 | 15 | 33 | 63 | Yes | Yes | Yes |
| | | | YQ | NWS | TAT | G99G99.Y99Y99. | N99N99.W99W99. | P99P99.T99T99. | | | | | | | |
| | | | | | DGV | Q99Q99. | S99S99. | A99A99.T99T99. | | | | | | | |
| | | | | | FG | | | D99D99.G99G99. | | | | | | | |
| | | | | | | | | V99V99.F99F99. | | | | | | | |
| | | | | | | | | G99G99. | | | | | | | |
| RC | 116 | 5 | SHL | YAA | DDS | S99S99.H99H99. | Y99Y99.A99A99. | D99D99.D99D99. | 21 | 15 | 27 | 63 | Yes | Yes | Yes |
| | | | ARG | AF | NCV | L99L99.A99A99. | A99A99.A99A99. | S99S99.N99N99. | | | | | | | |
| | | | C | | YAS | R99R99.G99G99. | F99F99. | C99C99.V99V99. | | | | | | | |
| | | | | | | C99C99. | | Y99Y99.A99A99. | | | | | | | |
| | | | | | | | | S99S99. | | | | | | | |
| RC | 118 | 5 | SVV | LVV | HIN | S99S99.V80B00. | L99L99.V99V99. | H99H99.I99I99. | 20 | 15 | 18 | 53 | Yes | Yes | Yes |
| | | | YVC | NR | RWI | V99V99.Y99Y99. | V99V99.N99N99. | N99N99.R99R99. | | | | | | | |
| | | | Y | | | V99V99.C99C99. | R99R99. | W99W99.I99I99. | | | | | | | |
| | | | | | | Y99Y99. | | | | | | | | | |
| RC | 45 | 5 | EMG | FRC | SWA | E99E99.M99M99. | F99F99.R99R99. | S99S99.W99W99. | 12 | 15 | 39 | 66 | Yes | Yes | Yes |
| | | | V | MY | RQH | G99G99.V99V99. | C99C99.M99M99. | A99A99.R99R99. | | | | | | | |
| | | | | | DPS | | Y99Y99. | Q99Q99.H99H99. | | | | | | | |
| | | | | | CQY | | | D99D99.P99P99. | | | | | | | |
| | | | | | A | | | S99S99.C99C99. | | | | | | | |
| | | | | | | | | Q99Q99.Y99Y99. | | | | | | | |
| | | | | | | | | A99A99. | | | | | | | |
| RC | 52 | 5 | QHS | MW | TSN | Q99Q99.H99H99. | M99M99.W99W99. | T99T99.S99S99. | 21 | 15 | 18 | 54 | Yes | Yes | Yes |
| | | | SFF | GIF | FFV | S99S99.S99S99. | G99G99.I99I99. | N99N99.F99F99. | | | | | | | |
| | | | C | | | F99F99.F99F99. | F99F99. | F99F99.V99V99. | | | | | | | |
| | | | | | | C99C99. | | | | | | | | | |
| RC | 70 | 5 | LVW | WFE | INM | L99L99.V99V99. | W99W99.F99F99. | B00 | 21 | 15 | 0 | 36 | Yes | Yes | Yes |
| | | | ITCP | SR | HGG | W99W99.I99I99. | E99E99.S99S99. | | | | | | | | |
| | | | | | | T99T99.C99C99. | R99R99. | | | | | | | | |
| | | | | | | P99P99. | | | | | | | | | |
| RC | 71 | 5 | EFD | TLLL | NGI | E99E99.F99F99. | T99T99.L99L99. | N99N99.G99G99. | 18 | 15 | 27 | 60 | Yes | Yes | Yes |
| | | | VW | R | TAT | D99D99.V99V99. | L99L99.L99L99. | I99I99.T99T99. | | | | | | | |
| | | | G | | CFC | W99W99.G99G99. | R99R99. | A99A99.T99T99. | | | | | | | |
| | | | | | | | | C99C99.F99F99. | | | | | | | |
| | | | | | | | | C99C99. | | | | | | | |
| RC | 79 | 5 | GNS | PVA | F | G99G99.N99N99. | P99P99.V99V99. | F99F99. | 21 | 15 | 3 | 39 | Yes | Yes | Yes |
| | | | RDR | GR | | S99S99.R99R99. | A99A99.G99G99. | | | | | | | | |
| | | | T | | | D99D99.R99R99. | R99R99. | | | | | | | | |
| | | | | | | T99T99. | | | | | | | | | |
| RC | 86 | 5 | YDS | RGG | NYL | Y99Y99.D99D99. | R99R99.G99G99. | N99N99.Y99Y99. | 21 | 15 | 18 | 54 | Yes | Yes | Yes |
| | | | SVG | LY | SLC | S99S99.S99S99. | G99G99.L99L99. | L99L99.S99S99. | | | | | | | |
| | | | G | | | V99V99.G99G99. | Y99Y99. | L99L99.C99C99. | | | | | | | |
| | | | | | | G99G99. | | | | | | | | | |
| RC | 87 | 5 | MEV | RGR | HTS | M99M99.E99E99. | R99R99.G99G99. | H80B00.T80B00. | 21 | 15 | 12 | 48 | Yes | Yes | Yes |
| | | | VSR | SY | ELS | V99V99.V99V99. | R99R99.S99S99. | S80B00.E80B00. | | | | | | | |
| | | | A | | | S99S99.R99R99. | Y99Y99. | L80B00.S80B00. | | | | | | | |
| | | | | | | A99A99. | | | | | | | | | |
| RC | 92 | 5 | GRA | GHG | CGP | G99G99.R99R99. | G99G99.H99H99. | C99C99.G99G99. | 9 | 15 | 9 | 33 | Yes | Yes | Yes |
| | | | | SV | | A99A99. | G99G99.S99S99. | P99P99. | | | | | | | |
| | | | | | | | V99V99. | | | | | | | | |
| RC | 96 | 5 | HFS | PLG | TLT | H99H99.F99F99. | P99P99.L99L99. | T99T99.L99L99. | 21 | 15 | 39 | 75 | Yes | Yes | Yes |
| | | | SRW | LF | RWY | S99S99.S99S99. | G99G99.L99L99. | T99T99.R99R99. | | | | | | | |
| | | | V | | FYD | R99R99.W99W99. | F99F99. | W99W99.Y99Y99. | | | | | | | |
| | | | | | SDS | V99V99. | | F99F99.Y99Y99. | | | | | | | |
| | | | | | F | | | D99D99.S99S99. | | | | | | | |
| | | | | | | | | D99D99.S99S99. | | | | | | | |
| | | | | | | | | F99F99. | | | | | | | |
| RC | 99 | 5 | LSN | HVF | TDV | L99L99.S99S99. | H99H99.V99V99. | T99T99.D99D99. | 21 | 15 | 18 | 54 | Yes | Yes | Yes |
| | | | TCVI | VR | YFR | N99N99.T99T99. | F99F99.V99V99. | V99V99.Y99Y99. | | | | | | | |
| | | | | | | C99C99.V99V99. | R99R99. | F99F99.R99R99. | | | | | | | |
| | | | | | | I99I99. | | | | | | | | | |
| RC | 2 | 5841 | VLV | ASY | LTS | V99B00.L99B00. | A99B00.S99B00. | B00 | 21 | 15 | 0 | 36 | No | No | Yes |
| | | | CYS | RY | PLS | V99B00.C99B00. | Y99B00.R99B00. | | | | | | | | |
| | | | Y | | DSS | Y99B00.S99B00. | Y99B00. | | | | | | | | |
| | | | | | | Y99B00. | | | | | | | | | |
| RC | 81 | 56 | LVV | ATL | LIPA | L99L99.V99V99. | A99A99.T99T99. | B00 | 21 | 15 | 0 | 36 | No | No | Yes |
| | | | TYE | RG | ESG | V99V99.T99T99. | L99L99.R99R99. | | | | | | | | |
| | | | R | | LVP | Y99Y99.E99E99. | G98B00. | | | | | | | | |
| | | | | | LVD | R99R99. | | | | | | | | | |
| RC | 23 | 53 | VCV | ETW | MRC | V99V99.C99C99. | E99E99.T99T99. | B00 | 21 | 15 | 0 | 36 | No | No | Yes |
| | | | SYT | MH | PPH | V99V99.S99S99. | W99W99.M99M99. | | | | | | | | |
| | | | H | | FFV | Y99Y99.T99T99. | H99H99. | | | | | | | | |
| | | | | | | H99H99. | | | | | | | | | |
| RC | 55 | 37 | ILC | CNA | AHL | I99199.L99L99. | C99C99.N99N99. | B00 | 21 | 15 | 0 | 36 | No | No | Yes |
| | | | MYE | RT | V | C99C99.M99M99. | A99A99.R99R99. | | | | | | | | |
| | | | Q | | | Y99Y99.E99E99. | T99T99. | | | | | | | | |
| | | | | | | Q86B00. | | | | | | | | | |
| RC | 40 | 20 | LLM | GCH | YLIR | L99L99.L99L99. | G99G99.C99C99. | B00 | 21 | 15 | 0 | 36 | No | No | Yes |
| | | | AYE | LV | HA | M95B00.A99A99. | H99H99.L99L99. | | | | | | | | |
| | | | C | | | Y99Y99.E99E99. | V99V99. | | | | | | | | |
| | | | | | | C99C99. | | | | | | | | | |
| RC | 54 | 18 | LRV | ANY | SHY | L99L99.R99R99. | A99A99.N99N99. | B00B00.B00B00. | 21 | 15 | 10 | 46 | No | No | Yes |
| | | | CYT | MN | TAL | V99V99.C99C99. | Y99Y99.M99M99. | B00B00.T44B00. | | | | | | | |
| | | | R | | VCN | Y99Y99.T99T99. | N99N99. | B00B00.B00B00. | | | | | | | |
| | | | | | V | R99R99. | | B00B00.B00B00. | | | | | | | |
| | | | | | | | | N44B00.V38B00. | | | | | | | |
| RC | 17 | 14 | FLC | ANL | LNM | F99F99.L99L99. | A99A99.N99N99. | B00 | 21 | 15 | 0 | 36 | No | No | Yes |
| | | | CYG | NR | PRD | C99C99.C99C99. | L99L99.N99N99. | | | | | | | | |
| | | | | | | Y99Y99.G99G99. | R99R99. | | | | | | | | |
| | | | | | | I99I99. | | | | | | | | | |
| RC | 117 | 7 | CVI | TPW | MCC | C99C99.V99V99. | T99T99.P99P99. | B00 | 21 | 15 | 0 | 36 | No | No | Yes |
| | | | NYH | VM | AAT | I99199.N99N99. | W99W99.V99V99. | | | | | | | | |
| | | | V | | FWD | Y99Y99.H99H99. | M99M99. | | | | | | | | |
| | | | | | P | V99V99. | | | | | | | | | |
| RC | 83 | 7 | DYG | YPG | LLS | D99D99.Y99Y99. | Y99Y99.P99P99. | B00 | 21 | 15 | 0 | 36 | No | No | Yes |
| | | | YCT | CH | RPK | G99G99.Y99Y99. | G99G99.C99C99. | | | | | | | | |
| | | | F | | RCF | C99C99.T99T99. | H99H99. | | | | | | | | |
| | | | | | VMV | F99F99. | | | | | | | | | |
| | | | | | M | | | | | | | | | | |
| RC | 90 | 6 | IPVF | PGF | NHT | I99I99.P99P99. | P99P99.G99G99. | B00 | 21 | 15 | 0 | 36 | No | No | Yes |
| | | | YCI | NL | L | V99V99.F99F99. | F99F99.N99N99. | | | | | | | | |
| | | | | | | Y99Y99.C99C99. | L99L99. | | | | | | | | |
| | | | | | | I99199. | | | | | | | | | |
| RC | 113 | 5 | TKF | YFL | TPS | T99T99.K99K99. | Y99Y99.F99F99. | T60V40.P60L40. | 21 | 15 | 20 | 56 | No | No | Yes |
| | | | EGL | QH | QVA | F99F99.E99E99. | L99L99.Q99Q99. | S60E40.Q60P40. | | | | | | | |
| | | | V | | SNF | G99G99.L99L99. | H99H99. | V60R40.A60V40. | | | | | | | |
| | | | | | Y | V99V99. | | S60T40.N60I40. | | | | | | | |
| | | | | | | | | F60Y40.Y60D40. | | | | | | | |
| RC | 119 | 5 | IYN | CGV | HYS | I99I99.Y99Y99. | C99C99.G99G99. | H99H99.Y99Y99. | 21 | 15 | 18 | 54 | No | No | Yes |
| | | | CKS | PF | DDP | N99N99.C99C99. | V99V99.P99P99. | S99S99.D99D99. | | | | | | | |
| | | | T | | | K99K99.S99S99. | F99F99. | D99D99.P99P99. | | | | | | | |
| | | | | | | T99T99. | | | | | | | | | |
| RC | 1 | 22616 | TRT | LIFI | TDD | T98B00.R99B00. | L99B00.I99B00. | T99B00.D99B00. | 21 | 15 | 18 | 54 | No | No | No |
| | | | ELR | M | FFN | T98B00.E99B00. | F99B00.I99B00. | D99B00.F99B00. | | | | | | | |
| | | | S | | | L99B00.R99B00. | M98B00. | F99B00.N99B00. | | | | | | | |
| | | | | | | S98B00. | | | | | | | | | |
| RC | 11 | 1623 | SSR | GTG | YLM | S97B00.S99B00. | G99B00.T99T99. | Y99B00.L98B00. | 21 | 15 | 18 | 54 | No | No | No |
| | | | SRF | YE | VTG | R99B00.S99B00. | G99B00.Y99B00. | M98B00.V98B00. | | | | | | | |
| | | | N | | | R99B00.F99B00. | E99B00. | T98B00.G98B00. | | | | | | | |
| | | | | | | N98B00. | | | | | | | | | |
| RC | 3 | 1109 | GNV | FHT | WPI | G99B00.N98B00. | F99B00.H99B00. | W99B00.P99B00. | 21 | 15 | 18 | 54 | No | No | No |
| | | | FLC | CG | LSA | V99B00.F99B00. | T99B00.C99B00. | I99B00.L99B00. | | | | | | | |
| | | | | | | L99B00.C99B00. | G99G99. | S99B00.A99B00. | | | | | | | |
| | | | | | | G99B00. | | | | | | | | | |
| RC | 12 | 845 | SYV | GYA | TIYG | S99B00.Y98B00. | G99B00.Y99B00. | T99B00.I99B00. | 21 | 15 | 18 | 54 | No | No | No |
| | | | NVW | IS | VY | V99B00.N99B00. | A99B00.I99B00. | Y99Y99.G99B00. | | | | | | | |
| | | | G | | | V99B00.W99B00. | S99B00. | V99B00.Y99B00. | | | | | | | |
| | | | | | | G99B00. | | | | | | | | | |
| RC | 13 | 553 | LRS | DVV | MTS | L99B00.R99R99. | D99D99.V99B00. | M99B00.T99B00. | 21 | 15 | 18 | 54 | No | No | No |
| | | | RVIT | SF | EVT | S99B00.R99B00. | V99V99.S99B00. | S99B00.E99B00. | | | | | | | |
| | | | | | | V99V99.I99B00. | F99F99. | V99B00.T99B00. | | | | | | | |
| | | | | | | T99B00. | | | | | | | | | |
| RC | 4 | 416 | ATIY | CQS | IINC | A99B00.T99T99. | C99B00.Q99Q99. | C56F43.I99I99. | 21 | 15 | 14 | 50 | No | No | No |
| | | | IYA | VR | LI | I96B00.Y99B00. | S99B00.V99B00. | N99B00.C56D43. | | | | | | | |
| | | | | | | I99B00.Y99B00. | R99R99. | L56D43.I56V43. | | | | | | | |
| | | | | | | A99B00. | | | | | | | | | |
| RC | 10 | 259 | RP | AYG | PSE | R99R99.P99B00. | A99A99.Y99Y99. | P99P99.S99S99. | 6 | 15 | 18 | 39 | No | No | No |
| | | | | CV | VFE | | G99G99.C98B00. | E99B00.V99V99. | | | | | | | |
| | | | | | | | V99V99. | F99F99.E99E99. | | | | | | | |
| RC | 24 | 254 | DRC | SGL | FLS | D99D99.R97B00. | S99S99.G99B00. | F99B00.L99B00. | 21 | 15 | 18 | 54 | No | No | No |
| | | | GTS | vw | AIE | C99B00.G99G99. | L99L99.V99B00. | S99B00.A97B00. | | | | | | | |
| | | | T | | | T99B00.S99B00. | W99B00. | I99B00.E99B00. | | | | | | | |
| | | | | | | T98B00. | | | | | | | | | |
| RC | 34 | 135 | SVC | YSN | QIN | S95B00.V99V99. | Y99Y99.S99S99. | Q99Q99.I99I99. | 21 | 15 | 18 | 54 | No | No | No |
| | | | YVY | LH | PML | C99C99.Y99Y99. | N99N99.L99L99. | N99N99.P99P99. | | | | | | | |
| | | | G | | | V99V99.Y99B00. | H99H99. | M99M99.L99L99. | | | | | | | |
| | | | | | | G99B00. | | | | | | | | | |
| RC | 35 | 124 | MLT | LRA | FQS | M99B00.L99L99. | L99L99.R99B00. | F99F99.Q99Q99. | 21 | 15 | 18 | 54 | No | No | No |
| | | | LSA | PT | SNL | T99T99.L99L99. | A99A99.P99P99. | S99S99.S99S99. | | | | | | | |
| | | | R | | | S99S99.A99A99. | T99T99. | N99N99.L99L99. | | | | | | | |
| | | | | | | R99R99. | | | | | | | | | |
| RC | 21 | 111 | SSIY | NNL | RIN | S99B00.S99S99. | N99N99.N99N99. | R99B00.I99I99. | 21 | 15 | 18 | 54 | No | No | No |
| | | | VYL | NS | CDL | I98B00.Y99B00. | L99L99.N99N99. | N99N99.C99C99. | | | | | | | |
| | | | | | | V99V99.Y99B00. | S99S99. | D99D99.L99L99. | | | | | | | |
| | | | | | | L99L99. | | | | | | | | | |
| RC | 42 | 86 | ITVV | CCG | PLII | I97B00.T99T99. | C99C99.C99C99. | P98B00.L98B00. | 21 | 15 | 27 | 63 | No | No | No |
| | | | VGS | EP | QGC | V99V99.V99V99. | G99G99.E99E99. | I97B00.P98B00. | | | | | | | |
| | | | | | CY | V97B00.G99G99. | P98B00. | Q98B00.G98B00. | | | | | | | |
| | | | | | | S99S99. | | C97B00.C98B00. | | | | | | | |
| | | | | | | | | Y98B00. | | | | | | | |
| RC | 32 | 76 | RCV | GDG | NPF | R99R99.C99C99. | G99G99.D99D99. | N99N99.P99P99. | 21 | 15 | 30 | 66 | No | No | No |
| | | | IVG | FD | LGR | V99V99.I99I99. | G99G99.F99F99. | F98B00.L99L99. | | | | | | | |
| | | | C | | FAG | V99V99.G99G99. | D99D99. | G98B00.R99R99. | | | | | | | |
| | | | | | T | C99C99. | | F99F99.A99A99. | | | | | | | |
| | | | | | | | | G99G99.T99T99. | | | | | | | |
| RC | 36 | 60 | WM | GNV | SGM | W99W99.M99M99. | G99G99.N99N99. | S99S99.G99G99. | 21 | 15 | 27 | 63 | No | No | No |
| | | | FVL | PN | AWI | F99F99.V99V99. | V99V99.P99P99. | M99M99.A99A99. | | | | | | | |
| | | | SS | | YPI | L98B00.S99S99. | N99N99. | W99W99.I99I99. | | | | | | | |
| | | | | | | S99S99. | | Y99Y99.P99P99. | | | | | | | |
| | | | | | | | | I99I99. | | | | | | | |
| RC | 49 | 55 | HLV | ELS | QPS | H99H99.L99L99. | E99E99.L99L99. | Q99Q99.P99P99. | 21 | 15 | 18 | 54 | No | No | No |
| | | | PVA | RW | CYQ | V99V99.P98B00. | S99S99.R98B00. | S99S99.C99C99. | | | | | | | |
| | | | T | | | V98B00.A96B00. | W99W99. | Y99Y99.Q99Q99. | | | | | | | |
| | | | | | | T98B00. | | | | | | | | | |
| RC | 14 | 42 | LLFT | RAA | PCTI | L99L99.L99L99. | R99R99.A99A99. | P99P99.C99C99. | 12 | 15 | 18 | 45 | No | No | No |
| | | | | PS | SG | F99F99.T99T99. | A99A99.P99P99. | T99T99.I99I99. | | | | | | | |
| | | | | | | | S99S99. | S99S99.G99G99 | | | | | | | |
| RC | 16 | 39 | LAL | IGG | DTM | L99L99.A99A99. | I99I99.G99G99. | D99D99.T99T99. | 21 | 15 | 30 | 66 | No | No | No |
| | | | ARV | CL | PNY | L99L99.A99A99. | G99G99.C99C99. | M97B00.P99P99. | | | | | | | |
| | | | S | | YPM | R97B00.V99V99. | L99L99. | N99N99.Y99Y99. | | | | | | | |
| | | | | | | S99S99. | | I99I99.Y99Y99. | | | | | | | |
| | | | | | | | | P99P99.M99M99. | | | | | | | |
| RC | 60 | 39 | ILVG | VRD | QRS | I97B00.L99L99. | V99V99.R99R99. | Q99Q99.R99R99. | 21 | 15 | 27 | 63 | No | No | No |
| | | | IYW | WS | VFG | V99V99.G99G99. | D99D99.W99W99. | S99S99.V99V99. | | | | | | | |
| | | | | | SSG | I99I99.Y97B00. | S99S99. | F99F99.G99G99. | | | | | | | |
| | | | | | | W99W99. | | S99S99.S99S99. | | | | | | | |
| | | | | | | | | G99G99. | | | | | | | |
| RC | 48 | 32 | NIG | GW | FAS | N90B00.I9999. | G99G99.W99W99. | F96B00.A96B00. | 21 | 15 | 18 | 54 | No | No | No |
| | | | PCM | SSS | VLK | G99G99.P96B00. | S99S99.S99S99. | S96B00.V96B00. | | | | | | | |
| | | | C | | | C99C99.M96B00. | S99S99. | L96B00.K96B00. | | | | | | | |
| | | | | | | C99C99. | | | | | | | | | |
| RC | 26 | 31 | ESQ | SVH | SYL | E99E99.S99S99. | S99S99.V99V99. | S99S99.Y99Y99. | 21 | 15 | 27 | 63 | No | No | No |
| | | | ALR | cc | GYIF | Q99Q99.A99A99. | H99H99.C99C99. | L99L99.G99G99. | | | | | | | |
| | | | V | | PE | L99L99.R99R99. | C99C99. | Y99Y99.I99I99. | | | | | | | |
| | | | | | | V99V99. | | F99F99.P99P99. | | | | | | | |
| | | | | | | | | E99E99. | | | | | | | |
| RC | 44 | 27 | AIG | CCF | NAH | A99A99.I99I99. | C99C99.C99C99. | N99N99.A99A99. | 21 | 15 | 18 | 54 | No | No | No |
| | | | CNG | CS | QYF | G99G99.C99C99. | F99F99.C99C99. | H99H99.Q99Q99. | | | | | | | |
| | | | L | | | N99N99.G99G99. | S99S99. | Y99Y99.F99F99. | | | | | | | |
| | | | | | | L99L99. | | | | | | | | | |
| RC | 104 | 26 | VLK | IFQ | YHG | V99V99.L99L99. | I99I99.F99F99. | Y99Y99.H99H99. | 21 | 15 | 15 | 51 | No | No | No |
| | | | SML | NR | PG | K99K99.S99S99. | Q99Q99N99N99. | G99G99.P99P99. | | | | | | | |
| | | | L | | | M99M99.L99L99. | R99R99. | G99G99. | | | | | | | |
| | | | | | | L99L99. | | | | | | | | | |
| RC | 46 | 23 | DTV | LLS | IINC | D99D99.T99T99. | L99L99.L99L99. | I99I99.I99I99. | 21 | 15 | 18 | 54 | No | No | No |
| | | | YLL | SS | TK | V99V99.Y99Y99. | S99S99.S99S99. | N99N99.C99C99. | | | | | | | |
| | | | V | | | L99L99.L99L99. | S99S99. | T99T99.K99K99. | | | | | | | |
| | | | | | | V99V99. | | | | | | | | | |
| RC | 30 | 21 | PEIS | SYS | FAS | P99P99.E99E99. | S99S99.Y99Y99. | F99F99.A99A99. | 21 | 15 | 18 | 54 | No | No | No |
| | | | LIC | LR | SCC | I95B00.S99S99. | S99S99.L99L99. | S99S99S99S99. | | | | | | | |
| | | | | | | L99L99.I95B00. | R99R99. | C99C99.C99C99. | | | | | | | |
| | | | | | | C99C99. | | | | | | | | | |
| RC | 33 | 20 | CHS | PMG | RMR | C99C99.H99H99. | P99P99.M99M99. | R99R99.M99M99. | 21 | 15 | 39 | 75 | No | No | No |
| | | | GAL | VA | TPC | S95B00.G99G99. | G99G99.V99V99. | R99R99.T99T99. | | | | | | | |
| | | | Y | | LML | A95B00.L99L99. | A99A99. | P99P99.C99C99. | | | | | | | |
| | | | | | SGI | Y95B00. | | L99L99.M99M99. | | | | | | | |
| | | | | | S | | | L99L99.S99S99. | | | | | | | |
| | | | | | | | | G99G99.I99I99. | | | | | | | |
| | | | | | | | | S99S99. | | | | | | | |
| RC | 78 | 20 | PLA | FGD | YIQ | P99P99.L99L99. | F99F99.G99G99. | Y99Y99.I70B00. | 18 | 9 | 14 | 41 | No | No | No |
| | | | TSG | | GAC | A99A99.T99T99. | D95B00. | Q70B00.G70B00. | | | | | | | |
| | | | | | | S99S99.G99G99. | | A99A99.C70B00. | | | | | | | |
| RC | 95 | 20 | YG | IITS | SYL | Y99Y99.G99G99 | I99I99.I99I99. | S99S99.Y99Y99. | 6 | 15 | 18 | 39 | No | No | No |
| | | | | G | NSW | | T99T99.S99S99. | L99L99.N99N99. | | | | | | | |
| | | | | | | | G99G99. | S99S99.W99W99. | | | | | | | |
| RC | 28 | 17 | HNV | MHA | NHT | H99H99.N94B00. | M99M99.H99H99. | N99N99.H99H99. | 21 | 15 | 30 | 66 | No | No | No |
| | | | GVG | QS | DHY | V99V99.G99G99. | A94B00.Q99Q99. | T99T99.D99D99. | | | | | | | |
| | | | T | | IFPS | V99V99.G99G99. | S99S99. | H99H99.Y99Y99. | | | | | | | |
| | | | | | | T99T99. | | I99I99.F99F99. | | | | | | | |
| | | | | | | | | P99P99.S99S99. | | | | | | | |
| RC | 25 | 13 | VCY | ANQ | GPD | V99V99.C99C99. | A99A99.N99N99. | G99G99.P99P99. | 21 | 15 | 30 | 66 | No | No | No |
| | | | TGW | PR | ILSF | Y99Y99.T99T99. | Q99Q99.P99P99. | D99D99.I99I99. | | | | | | | |
| | | | D | | YGC | G99G99.W99W99. | R99R99. | L99L99.S99S99. | | | | | | | |
| | | | | | | D99D99. | | F99F99.Y99Y99. | | | | | | | |
| | | | | | | | | G92B00.C99C99. | | | | | | | |
| RC | 77 | 9 | VLIC | YAV | WPD | V99V99.L99L99. | Y99Y99.A99A99. | W99W99.P99P99. | 21 | 15 | 18 | 54 | No | No | No |
| | | | RTA | RF | CCN | I99I99.C99C99. | V99V99.R99R99. | D99D99.C99C99. | | | | | | | |
| | | | | | | R99R99.T99T99. | F99F99. | C99C99.N99N99. | | | | | | | |
| | | | | | | A99A99. | | | | | | | | | |
| RC | 94 | 9 | VHIT | VPG | SGL | V99V99.H99H99. | V99V99.P99P99. | S99S99.G99G99. | 21 | 15 | 26 | 62 | No | No | No |
| | | | RFV | QR | SFG | I99I99.T99T99. | G99G99.Q99Q99. | L99L99.S99S99. | | | | | | | |
| | | | | | AVR | R99R99.F99F99. | R99R99. | F99F99.G99G99. | | | | | | | |
| | | | | | | V99V99. | | A77B00.V99V99. | | | | | | | |
| | | | | | | | | R99R99. | | | | | | | |
| RC | 50 | 8 | RLR | QYG | PDL | R99R99.L99L99. | Q99Q99.Y99Y99. | P99P99.D99D99. | 12 | 15 | 30 | 57 | No | No | No |
| | | | P | RI | VIYL | R99R99.P99P99. | G99G99.R99R99. | L99L99.V99V99. | | | | | | | |
| | | | | | MPN | | I99I99. | I99I99.Y99Y99. | | | | | | | |
| | | | | | | | | L99L99.M99M99. | | | | | | | |
| | | | | | | | | P99P99.N99N99. | | | | | | | |
| RC | 112 | 7 | IDA | NIR | WIT | I99I99.D99D99. | N99N99.I99I99. | W99W99.I99I99. | 21 | 15 | 18 | 54 | No | No | No |
| | | | HDS | RG | CES | A99A99.H99H99. | R99R99.R99R99. | T99T99.C85B00. | | | | | | | |
| | | | V | | | D99D99.S99S99. | G99G99. | E99E99.S99S99. | | | | | | | |
| | | | | | | V99V99. | | | | | | | | | |
| RC | 114 | 6 | VLS | VGA | TFS | V99V99.L83B00. | V99V99.G99G99. | T99T99.F99F99. | 21 | 5 | 18 | 54 | No | No | No |
| | | | SEF | GW | TRY | S99S99.S99S99. | A99A99.G99G99. | S99S99.T99T99. | | | | | | | |
| | | | T | | | E99E99.F99F99. | W99W99. | R99R99.Y99Y99. | | | | | | | |
| | | | | | | T83B00. | | | | | | | | | |
| RC | 120 | 6 | DMY | MFS | YGS | D83B00.M99M99. | M99M99.F99F99. | Y99Y99.G99G99. | 21 | 15 | 30 | 66 | No | No | No |
| | | | GSL | DI | TMT | Y99Y99.G99G99. | S99S99.D99D99. | S99S99.T99T99. | | | | | | | |
| | | | T | | GCH | S99S99.L99L99. | I99I99. | M99M99.T99T99. | | | | | | | |
| | | | | | C | T99T99. | | G99G99.C99C99. | | | | | | | |
| | | | | | | | | H99H99.C99C99. | | | | | | | |
| RC | 103 | 5 | NMV | NWL | ALL | N99N99.M99M99. | N99N99.W99W99. | A99A99.L80B00. | 21 | 12 | 14 | 47 | No | No | No |
| | | | YMK | H | VRE | V99V99.Y99Y99. | L99L99.H99H99. | L99L99.V80B00. | | | | | | | |
| | | | H | | | M99M99.K99K99. | | R80B00.E80B00. | | | | | | | |
| | | | | | | H99H99. | | | | | | | | | |
| RC | 109 | 5 | SDT | YFR | ITF | S99S99.D99D99. | Y99Y99.F99F99. | I99I99.T99T99. | 21 | 15 | 9 | 45 | No | No | No |
| | | | VGL | HS | | T99T99V99V99. | R99R99.H99H99. | F99F99. | | | | | | | |
| | | | V | | | G99G99.L99L99. | S99S99. | | | | | | | | |
| | | | | | | V99V99. | | | | | | | | | |
| RC | 38 | 5 | NCN | RSP | WPD | N99N99.C99C99. | R99R99.S99S99. | W99W99.P99P99. | 21 | 15 | 18 | 54 | No | No | No |
| | | | RVIS | QV | CIV | N99N99.R99R99. | P99P99.Q99Q99. | D99D99.C99C99. | | | | | | | |
| | | | | | | V99V99.I99I99. | V99V99. | I99I99.V99V99. | | | | | | | |
| | | | | | | S99S99. | | | | | | | | | |
| RC | 57 | 5 | VSG | DFC | FLS | V99V99.S99S99. | D99D99.F99F99. | F99F99.L99L99. | 21 | 15 | 18 | 54 | No | No | No |
| | | | FRA | TG | CDA | G99G99.F99F99. | C99C99.T99T99. | S99S99.C99C99. | | | | | | | |
| | | | C | | | R99R99.A99A99. | G99G99. | D99D99.A99A99. | | | | | | | |
| | | | | | | C99C99. | | | | | | | | | |
| RC | 80 | 5 | FDA | VYY | KDM | F99F99.D99D99. | V99V99.Y99Y99. | K99K99.D99D99. | 21 | 15 | 9 | 45 | No | No | No |
| | | | AQA | VA | | A99A99.A99A99. | Y99Y99.V99V99. | M99M99. | | | | | | | |
| | | | W | | | Q99Q99.A99A99. | A99A99. | | | | | | | | |
| | | | | | | W99W99. | | | | | | | | | |

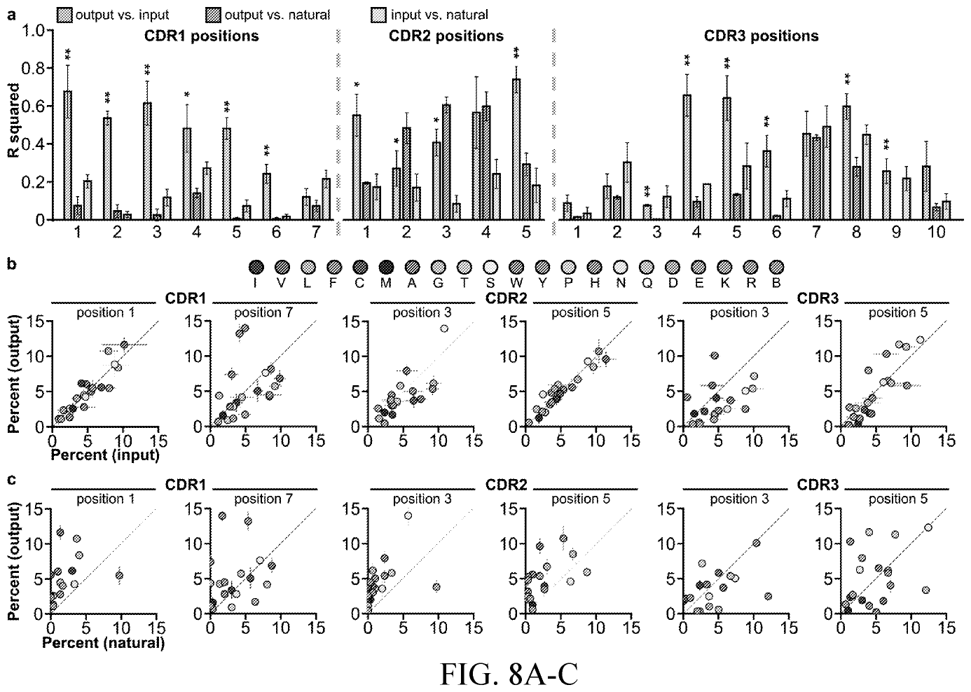

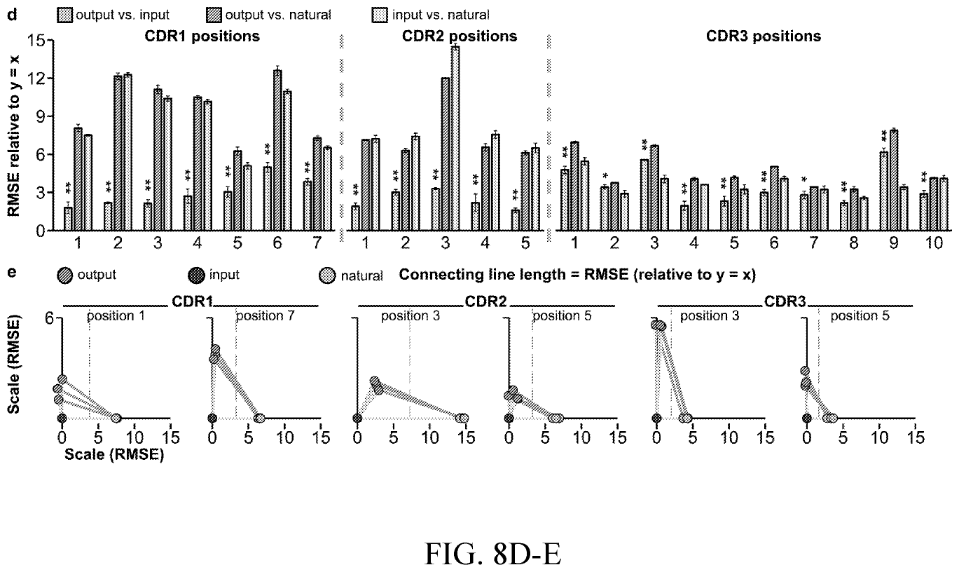

Applicants also identified amino acid changes that improve potency of the antibodies (see, e.g., FIG. 8). The present invention provides for CDRs and/or framework substitutions that increase potency (e.g., binding and/or neutralizing activity) of the antibodies. In certain embodiments, the VHH frameworks have 80, 85, 90, 95, 96, 97, 98, or 99% identity with the sequences described herein. In certain embodiments, antibodies are generated using one or more of the mutated CDR sequences and/or framework sequences described herein. Applicants specifically identified mutated antibodies having enhanced neutralizing activity (Table 9).

TABLE 9
Amino acid sequences of VHH variants and the mutations they contain.
Amino acid sequences of all VHH variants characterized in this study.

| Variant | | |
|---|---|---|
| Name | Sequence (SEQ ID NOS: 5811-5872) | Contained mutations |
| SR1 | EVQLVESGGGLVQAGDSLRLSCAASGKTGRSVIMGWFRQAPDKEREFVATITRGDDTTNY | NA |
| | ADSVKGRFTISRDDARNTVYLQMNSLKPEDTAVYYCAAVYFVNDCIVDYWGQGTQVTVSS | |
| SR1t2 | EVRLVESGGGLVLAGDSLRLSCAASGKTGRSVIMGWFRQAPDKEREFVATITRGDDTTNY | Q.3.R, Q.13.L |
| | ADSVKGRFTISRDDARNTVYLQMNSLKPEDTAVYYCAAVYFVNDCIVDYWGQGTQVTVSS | |
| SR2 | EVQLVESGGGLVQAGDSLRLSCAASGAFGSNVFMGWFRQAPGKEREFVASITPHHTVTDY | NA |
| | ADSVRGRFSISADSAKNTVYLQMNSLKPEDTAVYYCAAAIYRNASVLDYWGQGTQVTVSS | |
| SR2c3 | EVQLVESGGGLVQAGDSLRLSCAASGAFSSNVFMGWFRQAPGKEREFVASITPHHAVTDY | G.29.S, T.56.A, S.105.T |
| | ADSVRGRFSISADSAKNTVYLQMNSLKPEDTAVYYCAAAIYRNATVLDYWGQGTQVTVSS | |
| SR2c4 | EVQLVESGGGLVQAGDSLRLSCAASGAFSSNVFMGWFRQAPGKEREFVASITPHHAVTDY | G.29.S, T.56.A, R.102.C, S.105.T |
| | ADSVRGRFSISADSAKNTVYLQMNSLKPEDTAVYYCAAAIYCNATVLDYWGQGTQVTVSS | |
| SR2f3 | QVQLVESGGGLVQAGDSLRLSCAASGAFGSNVFMGWFRLAPGKEREFVASITPHHTVTDY | E.1.Q, Q.39.L, S.74.T |
| | ADSVRGRFSISADTAKNTVYLQMNSLKPEDTAVYYCAAAIYRNASVLDYWGQGTQVTVSS | |
| SR2t5 | QVQLVESGGGLVQAGDSLRLSCAASGAFSSNVFMGWFRQAPGKEREFVASITPHHAVTDY | G.29.S, T.56.A, S.105.T, E.1.Q, |
| | ADSVRGRFSISADTAKNTVYLQMNSLKPEDTAVYYCAAAIYRNATVLDYWGQGTQVTVSS | S.74.T |
| SR2t9 | QVQLVESGGGLVQAGDSLRLSCAATGAFSSNVFMGWFRLAPGKEREFVASITPHHAVTDY | E.1.Q, S.25.T, G.29.S, Q.39.L, |
| | ADSVRGRFSISADTAKNTVYLQMNALKPEDTAVYYCAAAIYCNATVLDYWGQGTQVTVSS | T.56.A, S.74.T, S.85.A, R.102.C, |
| | | S.105.T |
| SR4 | EVQLVESGGGLVQAGDSLRLSCAASGFQLSCYCMGWFRQAPGKEREFVASITQHAFRTYY | NA |
| | ADSVKGRFTISRDDARNTVYLQMNSLKPEDTAVYYCAAAETPNILVYENYWGQGTQVTVS | |
| | S | |
| SR4c1 | EVQLVESGGGLVQAGDSLRLSCAASGFQLSCYCMGWFRQAPGKEREFVASITQHAFRTYY | Y.107.H |
| | ADSVKGRFTISRDDARNTVYLQMNSLKPEDTAVYYCAAAETPNILVHENYWGQGTQVTVS | |
| | S | |
| SR4f3 | EVQLVVSGRGLVQAGDSLRLSCAASGFQLSCYCVGWFRQAPGKEREFVASITQHAFRTYY | E.6.V, G.9.R, M.34.V |
| | ADSVKGRFTISRDDARNTVYLQMNSLKPEDTAVYYCAAAETPNILVYENYWGQGTQVTVS | |
| | S | |
| SR4t4 | EVQLVVSGRGLVQAGDSLRLSCAASGFQLSCYCVGWFRQAPGKEREFVASITQHAFRTYY | Y.107.H, E.6.V, G.9.R, M.34.V |
| | ADSVKGRFTISRDDARNTVYLQMNSLKPEDTAVYYCAAAETPNILVHENYWGQGTQVTVS | |
| | S | |
| SR4t5 | EVQLVVSGRVLVQAGDSLRLSCAASGFQLSCYCVGWFRQAPGKEREFVASITQHAFRTYY | Y.107.H, E.6.V, G.9.R, M.34.V, |
| | ADSVKGRFTISRDDARNTVYLQMNSLKPEDTAVYYCAAAETPNILVHENYWGQGTQVTVS | G.10.V |
| | S | |
| SR4t6 | EVQLVVSGRVLVQAGDSLRLSCAASGFQLSCYCVGWFRQAPGKEREFVASITQHAFRTYY | Y.107.H, E.6.V, G.9.R, M.34.V, |
| | ADSVKGRFTISRDDARNTVYLQMNSLKPEDTAVYYCAAAETPNILVHESYWGQGTQVTVS | G.10.V, N.109.S |
| | S | |
| SR4t9 | KVQLVVSGRVLVQAGDSLRLSCAATGFQLSCYCVGWFRQAPGKELEFVASITQHAFRTYY | E.6.V, G.9.R, M.34.V, G.10.V, |
| | ADSVKGRFTISRDDARNTVYLQMNSLKPEDTAVYYCAAAETPNILVHESYWGQGTQVTVS | N.109.S, R.45.L, S.25.T, Y.107.H, |
| | S | E.1.K |
| SR4t13 | KVQLVVTGRVLVQAGDSLRLSCVATGFQLSCYCVGWFRQAPGKELEFVASITQHAFRTYY | E.1.K, E.6.V, S.7.T, G.9.R, |
| | ADSVKGRFTISRDDAQNTVYLQMNSLEPEDTAVYYCAAAETPNILVHESYWGQGTQVTVS | G.10.V, A.23.V, S.25.T, M.34.V, |
| | S | R.45.L, R.76.Q, K.87.E, Y.107.H, |
| | | N.109.S |
| SR6 | EVQLVESGGGLVQAGDSLRLSCAASGDCHVLWRMGWFRQAPGKEREFVAAISLDGLFTNY | NA |
| | ADSVRGRFSISADSAKNTVYLQMNSLKPEDTAVYYCAAMPFFDYWGQGTQVTVSS | |
| SR6c3 | EVQLVESGGGLVQAGDSLRLSCAASGDCHVLWRMGWFRQAPGKEREFVAAISLDGQFTNY | L.56.Q, M.99.L, F.102.L |
| | ADSVRGRFSISADSAKNTVYLQMNSLKPEDTAVYYCAALPFLDYWGQGTQVTVSS | |
| SR6f3 | QVQLVVSGGGLVQAGDSLRLSCAASGDCHVLWRMGWFRQAPGKEREFVAAISLDGLFTNY | E.1.Q, E.6.V, K.76.E |
| | ADSVRGRFSISADSAENTVYLQMNSLKPEDTAVYYCAAMPFFDYWGQGTQVTVSS | |
| SR6t4 | QVQLVESGGGLVQAGDSLRLSCAASGDCHVLWRMGWFRQAPGKEREFVAAISLDGQFTNY | L.56.Q, M.99.L, F.102.L, E.1.Q |
| | ADSVRGRFSISADSAKNTVYLQMNSLKPEDTAVYYCAALPFLDYWGQGTQVTVSS | |
| SR6t5 | QVQLVVSGGGLVQAGDSLRLSCAASGDCHVLWRMGWFRQAPGKEREFVAAISLDGQFTNY | L.56.Q, M.99.L, F.102.L, E.1.Q, |
| | ADSVRGRFSISADSAKNTVYLQMNSLKPEDTAVYYCAALPFLDYWGQGTQVTVSS | E.6.V |
| SR6t6 | QVQLVVSGGGLVQAGDSLRLSCAASGDCHVLWRMGWFRQAPGKEREFVAAISLDGQFTNY | L.56.Q, M.99.L, F.102.L, E.1.Q, |
| | ADSVRGRFSISADSAENTVYLQMNSLKPEDTAVYYCAALPFLDYWGQGTQVTVSS | E.6.V, K.76.E |
| SR6t9 | QVQLVVSGGGLVQAGDSLRLSCTASGDCHVLWRMGWFRQAPGKELEFVAAISLDGQFANY | E.1.Q, E.6.V, F.102.L, K.76.E, |
| | ADSVRGRFSISADSAENTVYLQMNSLKPEDTAVYYCAALPFLDYWGQGTQVTVSS | R.45.L, M.99.L, A.23.T, T.58.A, |
| | | L.56.Q |
| SR6t12 | QVQLVVSGGGLVRAGDSLRLSCTASGDCHVLWRMGWFRQASGKELEFVAAISLDGQFANY | E.1.Q, E.6.V, Q.13.R, A.23.T, |
| | ADSVRGRFSISADSAENTVYLQMNSLEPEDTAVYYCAALPFLDYWGQGTQVTVSS | P.41.S, R.45.L, L.56.Q, T.58.A, |
| | | K.76.E, K.87.E, M.99.L, F.102.L |
| SR8 | EVQLVESGGGLVQAGDSLRLSCAASGDYCMDCFMGWFRQAPGKEREFVAAITPYSFKTYY | NA |
| | ADSVKGRFTISRDDARNTVYLQMNSLKPEDTAVYYCAANPLDPNWTNSDYWGQGTQVTVS | |
| | S | |
| SR8c2 | EVQLVESGGGLVQAGDSLRLSCAASGDYCMDCFMGWFRQAPGKEREFVAAITPYGFKTYY | S.55.G, N.107.H |
| | ADSVKGRFTISRDDARNTVYLQMNSLKPEDTAVYYCAANPLDPNWTHSDYWGQGTQVTVS | |
| | S | |
| SR8t5 | EVQLVESGGGLVQAGDSLRLSCAASGDYCMDCFMGWFRQAPGKEREFVAAITPYGFKTYY | S.55.G, N.107.H, W.111.R, K.87.R, |
| | ADSVKGRFTFSRDDARNTVYLQMNSLRPEDTAVYYCAANPLDPNWTHSDYRGQGTQVTVS | I.70.F |
| | S | |
| SR12 | EVQLVESGGGLVQAGDSLRLSCAASGAIVHVFAMGWFRQAPGKEREFVASINLDNDATNY | NA |
| | ADSVKGRFTISRDDARNTVYLQMNSLKPEDTAVYYCNATIFMDSDLGDYWGQGTQVTVSS | |
| SR12c3 | EVQLVESGGGLVQAGDSLRLSCAASGAIVHVFAMGWFRQAPGKEREFVASINLDNDVTNY | A.57.V, M.102.T, D.105.G |
| | ADSVKGRFTISRDDARNTVYLQMNSLKPEDTAVYYCNATIFTDSGLGDYWGQGTQVTVSS | |
| SR12c5 | EVQLVESGGGLVQAGDSLRLSCAASGAIVHVFAMGWFRQAPGKEREFVASINLDNDVTNY | A.57.V, F.101.L, M.102.T, |
| | ADSVKGRFTISRDDARNTVYLQMNSLKPEDTAVYYCNATILTVSGLGDYWGQGTQVTVSS | D.103.V, D.105.G |
| SR12f3 | QVQLVVSGGGLVQAGDSLRLSCAASGAIVHVFAMGWFRQAPGKEREFVASINLDNDATNY | E.1.Q, E.6.V, Q.112.R |
| | ADSVKGRFTISRDDARNTVYLQMNSLKPEDTAVYYCNATIFMDSDLGDYWGRGTQVTVSS | |
| SR12t6 | QVQLVVSGGGLVQAGDSLRLSCAASGAIVHVFAMGWFRQAPGKEREFVASINLDNDVTNY | A.57.V, M.102.T, D.105.G, E.1.Q, |
| | ADSVKGRFTISRDDARNTVYLQMNSLKPEDTAVYYCNATIFTDSGLGDYWGRGTQVTVSS | E.6.V, Q.112.R |
| SR12t9 | QVQLVVSGRGLVQAGDSLRLSCVASGAIVHVFAMGWFRQAPGEEREFVASINLDNDATIY | E.1.Q, E.6.V, M.102.T, Q.112.R, |
| | ADSVKGRFTISRGDARNTVYLQMNSLKPEDTAVYYCNATIFTDSDLGDYWGRGTQVTVSS | G.9.R, D.73.G, K.43.E, A.23.V, |
| | | N.59.1 |
| SR15 | EVQLVESGGGLVQAGDSLRLSCAASGIGERCLFMGWFRQAPGKEREFVAAITAFSFRTYY | NA |
| | ADSVKGRFTISRDDARNTVYLQMNSLKPEDTAVYYCAALDTAALFPEFDYWGQGTQVTVS | |
| | S | |
| SR16 | EVQLVESGGGLVQAGDSLRLSCAASTFKLPCLMGWFRQTPGKEREFVAAISDHGGRTDYA | NA |
| | DSVKGRFTISRDDARNTVYLQMNSLKPEDTAVYYCNAVFIVKYEEELLVEAYWGQGTQVT | |
| | VSS | |
| SR17 | QVQLQESGGGLVQVGGSLRLSCTASGIWMGTRVMGWFRQAPGKEREFVATINGCYGTTDY | NA |
| | ADSVKGRFTISRDDARNTVYLQMNSLKPEDTAVYYCAALTRPPFDPLWDYWGQGTQVTVS | |
| | S | |
| SR18 | EVQLVESGGGLVQAGDSLRLSCAASGIMIGTQSMGWFRQAPGKEREFVASITANEMFTYY | NA |
| | ADSVRGRFSISADSAKNIVYLQMNSLKPEDTAVYYCAAITAMGWDYWGQGTQVTVSS | |
| SR19 | EVQLVESGGGLVQAGDSLRLSCAASGALCRRVYMGWFRQAPGKEREFVAAITCAGWRTDY | NA |
| | ADSVKGRFTISRDDARNTVYLQMNSLKPEDTAVYYCNAAVFASDGNVVGYWGQGTQVTVS | |
| | S | |
| SR20 | EVQLVESGGGLVQAGDSLRLSCAASGLCLSVLAMGWFRQAPGKEREFVAAINGSAAATYY | NA |
| | ADSVKGRFTISRDDARNTVYLQMNSLKPEDTAVYYCNAWLLTYSHDVMDYWGQGTQVTVS | |
| | S | |
| SR21 | QVQLQESGGGLVQAGDSLRLSCAASGRARGKVFMGWFRQAPGKEREFVASISCSGVDTNY | NA |
| | ADSVRGRFSISADSAKNTVYLQMNSLKPEDTAVYYCAAIFYDRTDRLSEYWGQGTQVTVS | |
| | S | |
| SR22 | EVQLVESGGGLVQAGDSLRLSCAASGTVIPYSMGWYRQAPGKEREFVAAINCMFLHTYYA | NA |
| | DSVKGRFTISRDDARNTVYLQMNSLKPEDTAVYYCNAPVPIPEESITDYWGQGTQVTVSS | |
| SR23 | EVQLVESGGGLVQAGDSLRLSCAASGSCIRWLSMGWFRQAPGKEREFVASITEYVKFTDY | NA |
| | ADSVRGRFSISADSAKNTVYLQMNSLKPEDTAVYYCAALENPHIASKYDYWGQGTQVTVS | |
| | S | |
| SR24 | EVQLVESGGGLVQAGDSLRLSCAASGIYLEAYGMGWFRQAPGKEREFVASISLYSFRTYY | NA |
| | ADSVKGRFTISRDDARNTVYLQMNSLKPEDTAVYYCAALQPTPSGWREAYWGQGTQVTVS | |
| | S | |
| SR25 | EVQLVESGGGLVQAGDSLRLSCAASGRLMNEVFMGWFRQAPGKEREFVASINRSAGTTDY | NA |
| | ADSVKGRFTISRDNAKNTVYLQMNSLKPEDTAVYYCAAAFYISFMVYDYWGQGTQVTVSS | |
| SR26 | EVQLVESGGGLVQAGDSLRLSCAASGLESLCGMGWFRQAPGKEREFVASISVGAIRTYYA | NA |
| | DSVRGRFSISADSAKNTVYLQMNSLKPEDTAVYYCAAWNYDCLGISDYWGQGTQVTVSS | |
| SR27 | EVQLVESGGGLVQAGDSLRLSCAASGGLHLALTMGWFRQAPGKEREFVASISQVLKVTNY | NA |
| | ADSVKGRFTISRDDARNTVYLQMNSLKPEDTAVYYCAAQILSHFDIVSGYWGQGTQVTVS | |
| | S | |
| SR28 | EVHLVESGGGLVQAGDSLRLSCAASGHARCSVIMGWFRQAPGKEREFVASINLAESVTYY | NA |
| | ADSVKGRFTISRDNAKNTVYLQMNSLKPEDTAVYYCAAVLPAMRLRYDYWGQGTQVTVSS | |
| SR29 | EVQLVESGGGLVQAGDSLRLSCAASGRADIYWMMGWFRQAPGKEREFVATINWRTSDTYY | NA |
| | ADSVRGRFSISADSAKNTVYLQMNSLKPEDTAVYYCNAVYLAGGVGYDSWGQGTQVTVSS | |
| SR30 | QVQLQESGGGLVQAGGSLRLSCAASGALSCGVFMGWFRQAPGKEREFVAAISGLGVDTDY | NA |
| | ADSVKGRFTISRDDARNTVYLQMNSLKPEDTAVYYCARAFFSSTGTVENYWGQGTQVTVS | |
| | S | |
| SR31 | EVQLVESGGGLVQAGDSLRLSCAASGFASDLCFMGWFRQAPGKEREFVATINCLGWTNYV | NA |
| | DSVKGRFTISRDDARNTVYLQMNSLKPEDTAVYYCAAYSHDLNSVVRDYWGQGTQVTVSS | |
| SR32 | EVQLVESGGGLVQAGDSLRLSCAASGFEQFDWRMGWYRQAPGKEREFVAAISSAYSTTNY | NA |
| | ADSVKGRFTISRDDARNTVYLQMNSLKPEDTAVYYCNAGTFLEDYFYRNYWGQGTQVTVS | |
| | S | |
| SR33 | EVQLVESGGGLVQAGDSLRLSCAASGNWSCVFQMGWFRQAPGKEREFVAAITQSPAVTDY | NA |
| | ADSVKGRFTISRDNAKNTVYLQMNSLKPEDTAVYYCAALACSLTGICNYWGQGTQVTVSS | |
| SR34 | EVQLVESGGGLVQAGDSLRLSCAASGRSHDCINMGWFRQAPGKEREFVAAINGFPIITYY | NA |
| | ADSVKGRFTISRDDARNTVYLQMNSLKPEDTAVYYCNAPVVLRRNIINYWGQGTQVTVSS | |
| SR35 | QVQLQESGGGLVQAGGSLRLSCAASGVSFHVFYMGWFRQAPGKEREFVASINNSGTHTYY | NA |
| | ADSVRGRFSISADSAKNTVYLQMNSLKLEDTAVYYCNAVSIVQGTTGDSWGQGTQVTVSS | |
| SR36 | EVQLVESGGGLVQAGDSLRLSCAASGGHWCSLTMGWFRQAPGKERELVAAINLAQYGTYY | NA |
| | ADSVKGRFTISRDDARNTVYLQMNSLKPEDTAVYYCAAAYATYNLDRGYSMGYWGQGTQV | |
| | TVSS | |
| SR37 | EVQLVESGGGLVQAGGSLRLSCAASGNPDPCVVMGWFRQAPGKEREFVASITAYSAVTYY | NA |
| | ADSVKGRFTISRDNAKNTVYLQMNSLKPEDTAVYYCAATYVSHPCHLYDYWGQGTQVTVS | |
| | S | |
| SR38 | EVQLVESGGGLVQAGDSLRLSCAASGSLYAVVWMGWFRQAPGKEREFVATISLCGYDTYY | NA |
| | ADSVKGRFTISRDDAHNTVYLQMNSLKPEDTAVYYCAVTCFVNHMKISEYWGQGTQVTVS | |
| | S | |
| SR6v1 | EVQLVESGGGLVQAGDSLRLSCAASGDSHVLWRMGWFRQAPGKEREFVAAISLDGQFTNY | C.28.S |
| | ADSVRGRFSISADSAKNTVYLQMNSLKPEDTAVYYCAALPFLDYWGQGTQVTVSS | |
| SR6v7 | EVQLVESGGGLVQAGDSLRLSCAASGDSYILWRMGWFRQAPGKEREFVAAISLDGQFTNY | C.28.S, H.29.Y, V.30.I |
| | ADSVRGRFSISADSAKNTVYLQMNSLKPEDTAVYYCAALPFLDYWGQGTQVTVSS | |
| SR6v9 | EVQLVESGGGLVQAGDSLRLSCAASGDSHVLWRMGWFRQAPGKEREFVAAISLDGQFTNY | C.28.S, N.77.Y |
| | ADSVRGRFSISADSAKYTVYLQMNSLKPEDTAVYYCAALPFLDYWGQGTQVTVSS | |
| SR6v10 | EVQLVESGGGLVQAGDSLRLSCAASGDSHVLWRMGWFRQAPGKELEFVAAISLDGQFTNY | C.28.S, R.45.L |
| | ADSVRGRFSISADSAKNTVYLQMNSLKPEDTAVYYCAALPFLDYWGQGTQVTVSS | |
| SR6v15 | EVQLVESGGGLVQAGDSLRLSCAASGDSYILWRMGWFRQAPGKEREFVAAISLDGQFTNY | C.28.S, N.77.Y, H.29.Y, V.30.I |
| | ADSVRGRFSISADSAKYTVYLQMNSLKPEDTAVYYCAALPFLDYWGQGTQVTVSS | |

In certain embodiments, antibodies prepared according to the present invention are substantially free of non-antibody protein. As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

In preferred embodiments, the antibodies of the present invention are monoclonal antibodies. As used herein, the term “monoclonal antibody” refers to a single antibody produced by any means, such as recombinant DNA technology. As used herein, the term “monoclonal antibody” also refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, VHH, single-chain antibodies, e.g., scFv, and single domain antibodies.

In certain embodiments, “humanized” forms of non-human antibodies contain amino acid residues in frame regions that resemble human antibody frame regions. In certain embodiments, frame regions of camelid antibodies or heavy chain antibodies are modified. In certain embodiments, humanized residues can be found in any human IGHV gene. In certain embodiments, the humanized residues are located in frame 2 or frame 4. In preferred embodiments, the humanized residues are located in frame 2 position 4, frame 2 position 11, frame 2 position 12, frame 2 position 14, frame 4 position 8. Humanized frames can be based on well characterized VHHs (Kirchhofer et al., 2010; Turner et al., 2014). These frames share high homology with the human IGHV3-23 or IGHJ4, but can be altered further as described herein (e.g., Frames 2 and 4).

In certain embodiments, “humanized” forms of non-human antibodies are chimeric antibodies that contain minimal sequence derived from non-human immunoglobulin (e.g., camelid). For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CHI domain; (v) the Fv fragment having the VL and VH domains of a single arm of an antibody; (vi) the dAb fragment (Ward et al., 341 Nature 544 (1989)) which consists of a VH domain or a VL domain that binds antigen; (vii) isolated CDR regions or isolated CDR regions presented in a functional framework; (viii) F(ab′)2 fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (VH) connected to a light chain variable domain (VL) in the same polypeptide chain (see, e.g., EP 404,097; WO 93/11161; Hollinger et al., 90 PNAS 6444 (1993)); (xi) “linear antibodies” comprising a pair of tandem Fd segments (VH-Ch1-VH-Ch1) which, together with complementary light chain polypeptides, form a pair of antigen binding regions (Zapata et al., Protein Eng. 8(10):1057-62 (1995); and U.S. Pat. No. 5,641,870).

In certain embodiments, the antibodies and CDRs of the present invention can be transferred to another antibody type (e.g., to the heavy chain of antibodies having heavy and light chains) to generate chimeric antibodies. It is intended that the term “antibody type” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “frame regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

In certain embodiments, the antibodies described herein or identified according to the methods described herein are blocking antibodies. As used herein, a “blocking” antibody or an antibody “antagonist” is one which inhibits or reduces biological activity of the antigen(s) it binds. In certain embodiments, the blocking antibodies or antagonist antibodies or portions completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

### Therapeutic Antibody Modifications

In certain example embodiments, the therapeutic antibodies of the present invention may be modified, such that they acquire advantageous properties for therapeutic use (e.g., stability and specificity), but maintain their biological activity. Therapeutic antibodies may be modified to increase stability or to provide characteristics that improve efficacy of the antibody when administered to a subject in vivo. As used herein in reference to therapeutic antibodies, the terms “modified”, “modification” and the like refer to one or more changes that enhance a desired property of the therapeutic antibody. “Modification” includes a covalent chemical modification that does not alter the primary amino acid sequence of the therapeutic antibody itself. Such desired properties include, for example, prolonging the in vivo half-life, increasing the stability, reducing the clearance, altering the immunogenicity or allergenicity, or cellular targeting. Changes to a therapeutic antibody that may be carried out include, but are not limited to, conjugation to a carrier protein, conjugation to a ligand, conjugation to another antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids. Modified therapeutic antibodies also include analogs. By “analog” is meant a molecule that is not identical, but has analogous functional or structural features. For example, a therapeutic antibody analog retains the biological activity of a corresponding antibody, while having certain biochemical modifications that enhance the analog's function relative to another antibody. Such biochemical modifications could increase the analog's protease resistance, membrane permeability, or half-life, without altering, for example, antigen binding. An analog may include an unnatural amino acid.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Modified antibodies (e.g., fusion proteins) may include a spacer or a linker. The terms “spacer” or “linker” as used in reference to a fusion protein refers to a peptide that joins the proteins comprising a fusion protein. Generally, a spacer has no specific biological activity other than to join or to preserve some minimum distance or other spatial relationship between the proteins. However, in certain embodiments, the constituent amino acids of a spacer may be selected to influence some property of the molecule such as the folding, net charge, or hydrophobicity of the molecule. Suitable linkers for use in an embodiment of the present invention are well known to those of skill in the art and include, but are not limited to, straight or branched-chain carbon linkers, heterocyclic carbon linkers, or peptide linkers. The linker is used to separate two peptides by a distance sufficient to ensure that, in a preferred embodiment, each peptide properly folds. Preferred peptide linker sequences adopt a flexible extended conformation and do not exhibit a propensity for developing an ordered secondary structure. Typical amino acids in flexible protein regions include Gly, Asn and Ser. Virtually any permutation of amino acid sequences containing Gly, Asn and Ser would be expected to satisfy the above criteria for a linker sequence. Other near neutral amino acids, such as Thr and Ala, also may be used in the linker sequence. Still other amino acid sequences that may be used as linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180.

The clinical effectiveness of protein therapeutics (e.g., antibodies) is often limited by short plasma half-life and susceptibility to protease degradation. Studies of various therapeutic proteins (e.g., filgrastim) have shown that such difficulties may be overcome by various modifications, including conjugating or linking the polypeptide sequence to any of a variety of non-proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG).

It is well known that the properties of certain proteins can be modulated by attachment of polyethylene glycol (PEG) polymers, which increases the hydrodynamic volume of the protein and thereby slows its clearance by kidney filtration. (See, e.g., Clark et al., J. Biol. Chem. 271: 21969-21977 (1996)). Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity. Therefore, it is envisioned that certain agents can be PEGylated (e.g., on peptide residues) to provide enhanced therapeutic benefits such as, for example, increased efficacy by extending half-life in vivo. In certain embodiments, PEGylation of the agents may be used to extend the serum half-life of the agents and allow for particular agents to be capable of crossing the blood-brain barrier. Thus, in one embodiment, PEGylating antibodies improve the pharmacokinetics and pharmacodynamics of the antibodies.

In regard to peptide PEGylation methods, reference is made to Lu et al., Int. J. Pept. Protein Res.43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., hit. J. Hematol. 68: 1-18 (1998); Roberts et al., J. Pharm. Sci. 87: 1440-45 (1998); and Tan et al., Protein Expr. Purif. 12: 45-52 (1998). Polyethylene glycol or PEG is meant to encompass any of the forms of PEG that have been used to derivatize other proteins, including, but not limited to, mono-(C1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG2-NHS-40k (Nektar); mPEG2-MAL-40k (Nektar), SUNBRIGHT GL2-400MA ((PEG)240 kDa) (NOF Corporation, Tokyo), SUNBRIGHT ME-200MA (PEG20 kDa) (NOF Corporation, Tokyo). The PEG groups are generally attached to the peptide (e.g., RBD) via acylation or alkylation through a reactive group on the PEG moiety (for example, a maleimide, an aldehyde, amino, thiol, or ester group) to a reactive group on the peptide (for example, an aldehyde, amino, thiol, a maleimide, or ester group).

The PEG molecule(s) may be covalently attached to any Lys, Cys, or K(CO(CH2)2SH) residues at any position in a peptide. In certain embodiments, the antibodies described herein can be PEGylated directly to any amino acid at the N-terminus by way of the N-terminal amino group. A “linker arm” may be added to a peptide to facilitate PEGylation. PEGylation at the thiol side-chain of cysteine has been widely reported (see, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. In certain embodiments, proteins are PEGylated through the side chains of a cysteine residue added to the N-terminal amino acid.

In exemplary embodiments, the PEG molecule(s) may be covalently attached to an amide group in the C-terminus of a peptide. In certain embodiments, the PEG molecule used in modifying an agent of the present invention is branched while in other embodiments, the PEG molecule may be linear. In particular aspects, the PEG molecule is between 1 kDa and 100 kDa in molecular weight. In further aspects, the PEG molecule is selected from 10, 20, 30, 40, 50, 60, and 80 kDa. In further still aspects, it is selected from 20, 40, or 60 kDa. Where there are two PEG molecules covalently attached to the agent of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the antibodies contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

The present disclosure also contemplates the use of PEG Mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the antibodies (e.g., Amunix' XTEN technology; Mountain View, CA). This obviates the need for an additional conjugation step during the manufacturing process. Moreover, established molecular biology techniques enable control of the side chain composition of the polypeptide chains, allowing optimization of immunogenicity and manufacturing properties.

Glycosylation can dramatically affect the physical properties of proteins and can also be important in protein stability, secretion, and subcellular localization (see, e.g., Solá and Griebenow, Glycosylation of Therapeutic Proteins: An Effective Strategy to Optimize Efficacy. BioDrugs. 2010; 24(1): 9-21). Proper glycosylation can be essential for biological activity. In fact, some genes from eukaryotic organisms, when expressed in bacteria (e.g., E. coli) which lack cellular processes for glycosylating proteins, yield proteins that are recovered with little or no activity by virtue of their lack of glycosylation. For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the original sequence.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants.

The present disclosure also contemplates the use of polysialylation, the conjugation of peptides and proteins to the naturally occurring, biodegradable a-(2→8) linked polysialic acid (“PSA”) in order to improve their stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of activity in vivo activity, and reduction in immunogenicity and antigenicity (see, e.g., G. Gregoriadis et al., Int. J. Pharmaceutics 300(1-2): 125-30). As with modifications with other conjugates (e.g., PEG), various techniques for site-specific polysialylation are available (see, e.g., T. Lindhout et al., PNAS 108(18)7397-7402 (2011)).

Additional suitable components and molecules for conjugation include, for example, thyroglobulin; albumins such as human serum albumin (HAS); tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Fusion of albumin to one or more antibodies of the present disclosure can, for example, be achieved by genetic manipulation, such that the DNA coding for HSA, or a fragment thereof, is joined to the DNA coding for the one or more antibodies. Albumin itself may be modified to extend its circulating half-life. Fusion of the modified albumin to one or more polypeptides can be attained by the genetic manipulation techniques described above or by chemical conjugation; the resulting fusion molecule has a half-life that exceeds that of fusions with non-modified albumin. (See WO2011/051489).

Several albumin-binding strategies have been developed as alternatives for direct fusion, including albumin binding through a conjugated fatty acid chain (acylation). Because serum albumin is a transport protein for fatty acids, these natural ligands with albumin-binding activity have been used for half-life extension of small protein therapeutics. For example, insulin determir (LEVEMIR), an approved product for diabetes, comprises a myristyl chain conjugated to a genetically-modified insulin, resulting in a long-acting insulin analog.

Another type of modification is to conjugate (e.g., link) one or more additional components or molecules at the N- and/or C-terminus of a polypeptide sequence, such as another protein, or a carrier molecule. Thus, an exemplary polypeptide sequence can be provided as a conjugate with another component or molecule. A conjugate modification may result in a polypeptide sequence that retains activity with an additional or complementary function or activity of the second molecule. For example, a polypeptide sequence may be conjugated to a molecule, e.g., to facilitate solubility, storage, in vivo or shelf half-life or stability, reduction in immunogenicity, delayed or controlled release in vivo, etc. Other functions or activities include a conjugate that reduces toxicity relative to an unconjugated polypeptide sequence, a conjugate that targets a type of cell or organ more efficiently than an unconjugated polypeptide sequence, or a drug to further counter the causes or effects associated with a disorder or disease as set forth herein.

The present disclosure contemplates the use of other modifications, currently known or developed in the future, of the polypeptides to improve one or more properties. One such method for prolonging the circulation half-life, increasing the stability, reducing the clearance, or altering the immunogenicity or allergenicity of a polypeptide of the present disclosure involves modification of the polypeptide sequences by hesylation, which utilizes hydroxyethyl starch derivatives linked to other molecules in order to modify the molecule's characteristics. Various aspects of hesylation are described in, for example, U.S. Patent Appln. Nos. 2007/0134197 and 2006/0258607.

In particular embodiments, the antibodies include a protecting group covalently joined to the N-terminal amino group. In exemplary embodiments, a protecting group covalently joined to the N-terminal amino group of the proteins reduces the reactivity of the amino terminus under in vivo conditions. Amino protecting groups include —C1-10 alkyl, —C1-10 substituted alkyl, C2-10 alkenyl, —C2-10 substituted alkenyl, aryl, —C1-6 alkyl aryl, —C(O)—(CH2)1-6-COOH, —C(O)—C1-6 alkyl, —C(O)-aryl, —C(O)—O—C1-6 alkyl, or —C(O)—O-aryl. In particular embodiments, the amino terminus protecting group is selected from the group consisting of acetyl, propyl, succinyl, benzyl, benzyloxycarbonyl, and t-butyloxycarbonyl. In other embodiments, deamination of the N-terminal amino acid is another modification that may be used for reducing the reactivity of the amino terminus under in vivo conditions.

Chemically modified compositions of the antibodies wherein the antibody is linked to a polymer are also included within the scope of the present invention. The polymer selected is usually modified to have a single reactive group, such as an active ester for acylation or an aldehyde for alkylation, so that the degree of polymerization may be controlled. Included within the scope of polymers is a mixture of polymers. Preferably, for therapeutic use of the end-product preparation, the polymer will be pharmaceutically acceptable. The polymer or mixture thereof may include but is not limited to polyethylene glycol (PEG), monomethoxy-polyethylene glycol, dextran, cellulose, or other carbohydrate-based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In other embodiments, the antibodies are modified by PEGylation, cholesterylation, or palmitoylation. The modification can be to any amino acid residue. In preferred embodiments, the modification is to the N-terminal amino acid of the antibodies, either directly to the N-terminal amino acid or by way coupling to the thiol group of a cysteine residue added to the N-terminus or a linker added to the N-terminus such as trimesoyl tris(3,5-dibromosalicylate (Ttds). In certain embodiments, the N-terminus of the antibodies comprise a cysteine residue to which a protecting group is coupled to the N-terminal amino group of the cysteine residue and the cysteine thiolate group is derivatized with N-ethylmaleimide, PEG group, cholesterol group, or palmitoyl group. In other embodiments, an acetylated cysteine residue is added to the N-terminus of the agents, and the thiol group of the cysteine is derivatized with N-ethylmaleimide, PEG group, cholesterol group, or palmitoyl group. In certain embodiments, the antibodies of the present invention consist of an amino acid sequence which is bound with a methoxypolyethylene glycol(s) via a linker.

Substitutions of amino acids may be used to modify an antibody of the present invention. The phrase “substitution of amino acids” as used herein encompasses substitution of amino acids that are the result of both conservative and non-conservative substitutions. Conservative substitutions are the replacement of an amino acid residue by another similar residue in a polypeptide. Typical but not limiting conservative substitutions are the replacements, for one another, among the aliphatic amino acids Ala, Val, Leu and Ile; interchange of Ser and Thr containing hydroxy residues, interchange of the acidic residues Asp and Glu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

One of skill in the art from this disclosure and the knowledge in the art will appreciate that there are a variety of ways in which to produce such therapeutic antibodies. In general, such therapeutic antibodies may be produced either in vitro or in vivo. Therapeutic antibodies may be produced in vitro as peptides or polypeptides, which may then be formulated into a pharmaceutical composition and administered to a subject. Such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed antibodies (e.g., with protein A or G). Alternatively, antibodies may be produced in vivo by introducing molecules (e.g., DNA, RNA, viral expression systems, and the like) that encode antibodies into a subject, whereupon the encoded therapeutic antibodies are expressed.

In certain embodiments, the antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect antibodies that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene or gene signature.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Administration of Therapeutic Antibodies

For therapeutic uses, the antibodies described herein may be administered systemically, for example, formulated in a pharmaceutically-acceptable buffer such as physiological saline. Preferable routes of administration include, for example, subcutaneous, intravenous, interperitoneal, intramuscular, or intradermal injections that provide continuous, sustained levels of the antibody in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound. For example, a therapeutic compound is administered at a dosage that is cytotoxic to a neoplastic cell.

Human dosage amounts can initially be determined by extrapolating from the amount of antibody used in mice, as a skilled artisan recognizes it is routine in the art to modify the dosage for humans compared to animal models. Of course, this dosage amount may be adjusted upward or downward, as is routinely done in such treatment protocols, depending on the results of the initial clinical trials and the needs of a particular patient.

The therapeutic regimens disclosed herein comprise administration of antibodies of the invention or pharmaceutical compositions thereof to the patient in a single dose or in multiple doses (e.g., 1, 2, 3, 4, 5, 6, 7, 8, 10, 15, 20, or more doses). In one aspect, the therapeutic regimens comprise administration of the antibodies of the invention or pharmaceutical compositions thereof in multiple doses. When administered in multiple doses, the antibodies are administered with a frequency and in an amount sufficient to treat SARS-CoV-2. For example, the frequency of administration ranges from once a day up to about four times a day. In another example, the frequency of administration ranges from about once a week up to about once every six weeks.

Significant progress has been made in understanding pharmacokinetics (PK), pharmacodynamics (PD), as well as toxicity profiles of therapeutic antibodies in animals and humans, which have been in commercial development for more than three decades (see, e.g., Vugmeyster et al., Pharmacokinetics and toxicology of therapeutic proteins: Advances and challenges, World J Biol Chem. 2012 Apr. 26; 3(4): 73-92). In certain embodiments, therapeutic antibodies are administered by parenteral routes, such as intravenous (IV), subcutaneous (SC) or intramuscular (IM) injection. Molecular size, hydrophilicity, and gastric degradation are the main factors that preclude gastrointestinal (GI) absorption of therapeutic proteins (see, e.g., Keizer, et al., Clinical pharmacokinetics of therapeutic monoclonal antibodies. Clin Pharmacokinet. 2010 August; 49(8):493-507). Pulmonary delivery with aerosol formulations or dry powder inhalers has been used for selected proteins, e.g., exubera™ (see, e.g., Scheuch and Siekmeier, Novel approaches to enhance pulmonary delivery of proteins and peptides. J Physiol Pharmacol. 2007 November; 58 Suppl 5(Pt 2):615-25). Intravitreal injections have been used for peptides and proteins that require only local activity (see, e.g., Suresh, et al., Ocular Delivery of Peptides and Proteins. In: Van Der Walle C., editor. Peptide and Protein Delivery. London: Academic Press; 2011. pp. 87-103). In certain embodiments, SC administration of therapeutic antibodies is often a preferred route. In particular, the suitability of SC dosing for self-administration translates into significantly reduced treatment costs.

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

As indicated above, the pharmaceutical compositions according to the invention may be in the form suitable for sterile injection. To prepare such a composition, the suitable antibodies are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate).

### Vector Delivery

The invention also provides a delivery system comprising one or more vectors or one or more polynucleotide molecules, the one or more vectors or polynucleotide molecules comprising one or more polynucleotide molecules encoding an antibody of the present invention.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids.

### Use of Antibodies for Detection

In certain embodiments, the antibody or antibody fragment is capable of use for detecting SARS-CoV-2 or variants thereof using an immunoassay. Immunoassay methods are based on the reaction of an antibody to its corresponding target or analyte and can detect the analyte in a sample depending on the specific assay format. Immunoassays have been designed for use with a wide range of biological sample matrices. Immunoassay formats have been designed to provide qualitative, semi-quantitative, and quantitative results.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

In certain embodiments, the antibody or antibody fragment is conjugated to a detectable label. Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Platforms and Methods for Generating Antibodies

In one aspect, the present invention provides a platform for generating antibodies. The platform is entirely in vitro and allows for efficiently screening and identification of CDRs capable of binding an antigen of interest. The platform provides for libraries of DNA sequences encoding antibodies and methods of generating said libraries. The platform provides for screening the library by ribosome display. The platform provides for identifying families of antibodies capable of binding to an antigen of interest. The platform provides for affinity maturation by mutating selected antibodies. The platform provides for validation of antibodies.

In certain embodiments, the platform utilizes a VHH library obtained by methods described further herein. In certain embodiments, the platform utilizes VHH frameworks randomized at all CDRs in the framework (e.g., CDR1, CDR2, and CDR3). Libraries can include a varying number of members, such as up to about 100 members, such as up to about 1,000 members, such as up to about 5,000 members, such as up to about 10,000 members, such as up to about 100,000 members, such as up to about 500,000 members, or even more than 500,000 members. In one example, the methods can involve providing a VHH library containing a large number of potential antibodies. Such libraries are then screened by the methods disclosed herein to identify those library members that display a desired characteristic activity (e.g., binding).

In certain embodiments, the library is generated by analyzing naturally occurring antibody frameworks (e.g., camelid heavy chain antibodies). Templates are then generated with selected frameworks. In certain embodiments, CDR regions are chosen having the most variation between different antibody frameworks. Sets of primer pairs are generated to randomly mutate each CDR sequence in each framework. Each CDR is randomized with a set of two pairs of primers corresponding to the entire framework sequence. For example, the first pair of primers amplifies a first half of the framework and the second pair of primers amplifies the second half of the framework directly adjacent to the start of the first amplicon. The set of primers for the first CDR can include primers hybridizing to each end of the framework (i.e., the first pair and second pair of primers each includes a primer specific to one end). The primers specific to the ends of the framework are preferably blocked from being ligated. In certain embodiments, the primers are blocked by including a hairpin sequence. The primers for randomizing in each primer pair (i.e., not the primers hybridizing to the ends) hybridize to a region that is not mutagenized and include a randomized sequence. The region for hybridization is selected such that the primer hybridizes under the PCR annealing conditions (e.g., 50-70° C.). The primers also include a randomized sequence corresponding to the number of amino acids in the region of the CDR to be mutagenized. Randomization schemes may include NNN, which uses all 64 codons; NNB, which uses 48 codons; NNK, which uses 32 codons; and MAX, which assigns equal probabilities to each of the 20 amino acids (where N=A/C/G/T, B=C/G/T, S=C/G, and K=G/T) (see, e.g., Nov, Y., Appl Environ Microbiol. 2012 January; 78(1): 258-262). In certain embodiments, randomization schemes are used that avoid stop codons.

In certain embodiments, the library is generated by using PCR and ligation for each CDR in the framework. In certain embodiments, the PCR primer pairs in each set generate two amplicons capable of being ligated in only one orientation due to the ends of the amplicon being blocked. The ligated PCR product from the previous step is used as the template for the successive CDR randomization steps. The randomized sequence may be present in one or both of the primer pairs for a CDR sequence (e.g., CDR3). In certain embodiments, the PCR for each randomization step is performed using a DNA polymerase without or having extremely weak strand displacement activity (e.g., Phusion® High-Fidelity DNA Polymerase, New England Biolabs). The term strand displacement describes the ability to displace downstream DNA encountered during synthesis, such as a downstream double stranded region (e.g., hairpin). The terms “weak” and “strong” relate to the strength of displacement as compared to an average activity. In certain embodiments, weak refers to an activity that is only slightly greater than no activity. In certain embodiments, weak displacement activity refers to less than 95% or 99% of an encountered DNA strand is displaced and strong displacement activity refers to greater than 95% or 99% of encountered DNA is displaced under normal reaction conditions. In certain embodiments, the CDR with the shortest sequence is randomized in the first or second cycle and the CDR with the longest sequence is randomized in the last step (e.g., CDR3).

In certain embodiments, the framework sequence includes a promoter sequence. The promoter sequence is preferably compatible with in vitro transcription/translation systems (e.g., T7 promoter). Thus, in certain embodiments, the library is transcribed into mRNAs encoding for each antibody framework. The mRNA can then be translated to generate the antibody polypeptide. In certain embodiments, library members are cloned into vectors including the promoter sequence.

In certain embodiments, the framework sequence does not include a stop codon, whereby the mRNA and translated protein is not released by ribosomes. In certain embodiments, the platform includes ribosome display (see, e.g., Zahnd, et al., Ribosome display: selecting and evolving proteins in vitro that specifically bind to a target. Nat Methods 4, 269-279 (2007)). As used herein ribosome display refers to an in vitro selection and evolution technology for proteins and peptides from large libraries. In certain embodiments, the antigen of interest (e.g., viral spike protein) is immobilized to a solid surface (i.e., surface-immobilized target), such as magnetic particles, latex beads, nanoparticles, macro-beads, membranes, microplates, array surfaces, dipsticks and a host of other devices that facilitate the capture of specific biomolecules. The solid surface is then used to select for translated antibody frameworks capable of binding the antigen of interest. The solid surface can be washed and mRNA can be isolated. The mRNA can be converted to cDNA by reverse transcription PCR (RT-PCR). In preferred embodiments, the PCR reaction in the RT-PCR is performed using a mixture of two DNA polymerases, wherein one type is a DNA polymerase without or having extremely weak strand displacement activity (e.g., Phusion® High-Fidelity DNA Polymerase, New England Biolabs) and the other type is a DNA polymerase with strong strand displacement activity (e.g., Deep Vent® DNA Polymerase, New England Biolabs). The cDNA can then be used as input for successive rounds of ribosome display. In preferred embodiments, 3 rounds are performed, however 1, 2, 3, 4, 5, 6, 7, 8, 9 or 10 or more rounds can be performed. In certain embodiments, the number of rounds is saturated when the same library members are isolated at each round.

In certain embodiments, the stringency of the washing and binding steps is adjusted to increase binding stringency. In certain embodiments, stringency is increased by increasing the ionic strength of the buffers. In certain embodiments, stringency is increased by adding or increasing the concentration of detergent in the buffers. The binding and washing is preferably performed at about 4° C., however stringency can be changed by increasing the temperature. In certain embodiments, binding time is adjusted. In certain embodiments, binding time in initial cycles of ribosome display may be longer and in successive cycles decreased to increase stringency. For example, antibodies that bind overnight can be identified in an early round. In certain embodiments, binding is performed overnight (about 12 hours), 4 hours, 3, hours, 2 hours, 1 hour or less than 1 minute. In certain embodiments, binding is performed in buffers containing Mg²⁺ ons at concentrations of 5 mM or less.

In certain embodiments, the framework includes a sequence encoding for an epitope tag in frame with the antibody sequence and at the C-terminus of the antibody. In certain embodiments, library members are cloned into vectors including the epitope tag sequence. Non-limiting examples of epitope tags includes poly-histidine, HA-tag, c-myc tag, and FLAG-tag. In certain embodiments, the epitope tag can be used to enrich for full length mRNA sequences. For example, the entire antibody with the epitope tag is encoded by only a full length mRNA. Thus, enriching for ribosomes expressing an antibody framework fused to an epitope tag will enrich for full-length mRNAs. In certain embodiments, enrichment is performed for one or more rounds. In certain embodiments, full length mRNA are enriched during the step of generating the library. In certain embodiments, the first or first and second CDRs are randomized. The randomized frameworks are then used in ribosome display followed by enrichment using a solid surface specific for binding to the epitope tag. The enriched mRNA are then converted to cDNA and used as input for randomizing the last CDR sequence.

The platform also includes computational steps capable of clustering antibodies having similar CDRs. In certain embodiments, clustering allows for families of related antibodies to be identified, such that one or a few representative antibodies from each family can be further assayed or validated to determine binding or neutralizing activity of different families. In certain embodiments, every family identified is further validated. In certain embodiments, 1, 2, 3, 4, 5, 6, 7, 8, 9, 10 or 20 or more antibodies clustered in a family are validated.

In certain embodiments, antibody frameworks identified using a randomized library are further mutated by one or more rounds of affinity maturation. Affinity maturation refers to the introduction of random mutations across the full length of selected VHH DNA sequences (i.e., also including frame regions) and performing ribosome display with the antigen of interest. In certain embodiments, binding during ribosome display is performed for 1 minute or less.

In certain embodiments, the platform includes assays capable of validating antibody binding and neutralization activity. In certain embodiments, the validation assay is an immunoassay as described further herein (e.g., ELISA, radioimmunoassay, fluorescent, chemiluminescence, fluorescence resonance energy transfer (FRET) or time resolved-FRET (TR-FRET) immunoassays, Western blot, immunoprecipitation, immunocytochemistry, immunohistochemistry, or flow cytometry). These immunoassay formats have been designed to provide qualitative, semi-quantitative, and quantitative results. Quantitative results may be generated by determining the concentration of analyte detected by an antibody. Quantitative results may be generated through the use of a standard curve created with known concentrations of the specific analyte to be detected. The response or signal from an unknown sample is plotted onto the standard curve, and a quantity or value corresponding to the target in the unknown sample is established.

Viral neutralizing assays can be used to validate identified antibody frameworks. In certain embodiments, the assay uses live virus. In certain embodiments, the assay uses pseudotyped virus particles (see, e.g., Gentili, 2015). In certain embodiments, the neutralizing assay is performed without live virus (see, e.g., Tan, C. W., Chia, W. N., Qin, X. et al. A SARS-CoV-2 surrogate virus neutralization test based on antibody-mediated blockage of ACE2—spike protein—protein interaction. Nat Biotechnol 38, 1073-1078 (2020).

Further embodiments are illustrated in the following Examples which are given for illustrative purposes only and are not intended to limit the scope of the invention.

### EXAMPLES

### Example 1—a Cell-Free Antibody Engineering Platform Rapidly Generates SARS-CoV-2 Neutralizing Antibodies

Here, Applicants developed an integrated platform for in vitro VHH domain antibody engineering distinct from previous systems that integrates a novel design and generation method for CDR-randomized VHH libraries, optimized ribosome display and selection cycle with built-in background reduction, and a computational approach to perform global binder prediction from post-selection libraries. Applicants named this platform CeVICA (Cell-free VHH Identification using Clustering Analysis). CeVICA enabled Applicants to rapidly generate a list of more than 800 predicted binder families targeting SARS-CoV-2 spike receptor binding domain and engineer potent neutralizing antibodies against SARS-CoV-2 in response to an ongoing global pandemic caused by the virus (Cohen, 2020; Zhou et al., 2020).

### Development of CeVICA

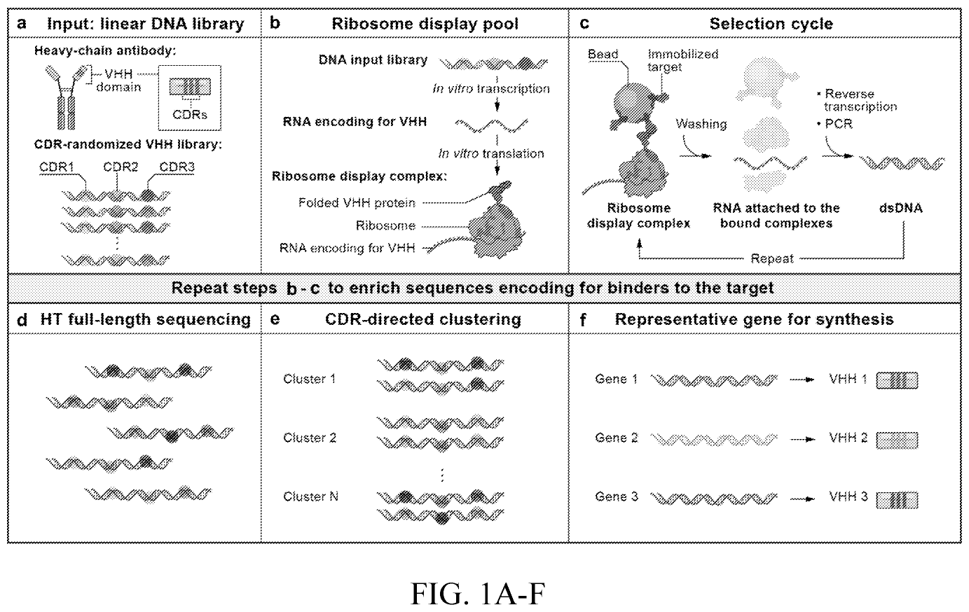

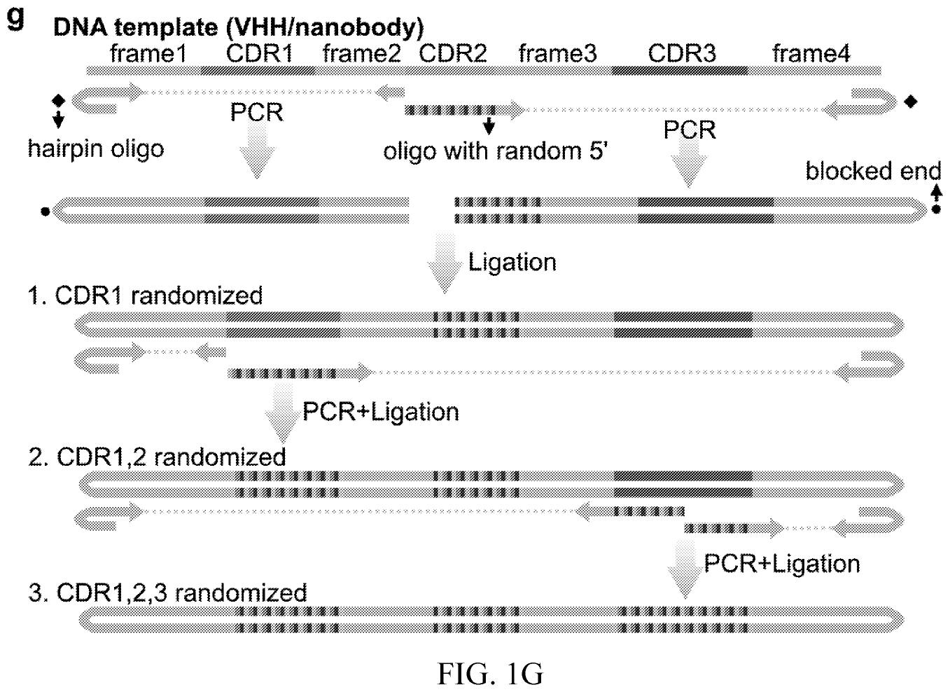

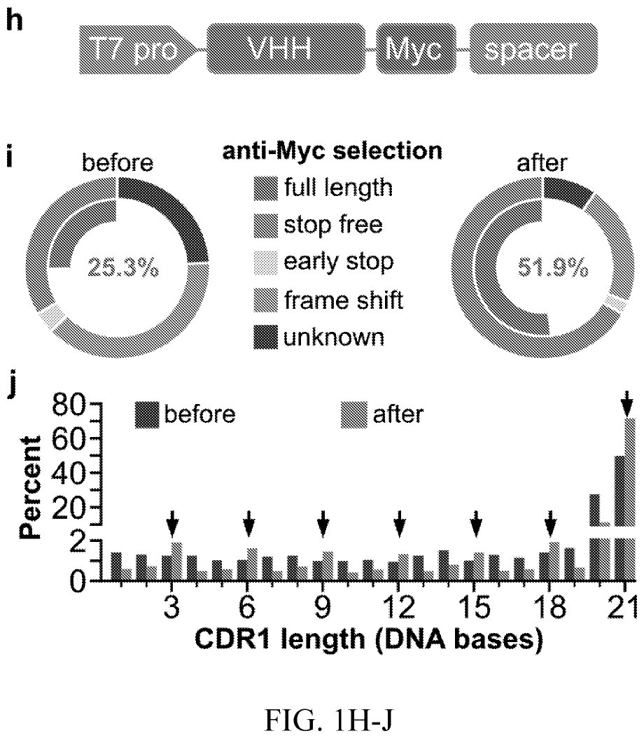

To leverage the advantages of cell-free display, Applicants developed CeVICA (Cell-free VHH Identification using Clustering Analysis) (FIG. 1), an integrated platform for in vitro VHH domain antibody engineering, distinct from previous systems^{7,8,14} in that it combines a new design and generation method for CDR-randomized VHH/nanobody libraries, optimized ribosome display based selection cycle with built-in background reduction, and a computational approach to perform global binder prediction from post-selection libraries. CeVICA first takes a linear DNA library as input, in which each sequence is unique and encodes for an artificial nanobody with three fully randomized CDRs, and where the 5′ and 3′ ends of the DNA molecules contain elements required for in vitro ribosome display (FIG. 1a, Methods). Next, CeVICA uses ribosome display to link genotype (RNAs transcribed from DNA input library that are stop codon free, and stall ribosome at the end of the transcript) and phenotype (folded nanobody protein tethered to ribosomes due to the lack of stop codon in the RNA) (FIG. 1b, Methods). In each selection cycle (FIG. 1c, Methods), the displaying ribosomes bind to an immobilized target, followed by RT-PCR of the RNA attached to the bound ribosomes, which leads to double stranded DNA, which is then in vitro transcribed/translated in a new round of ribosome display. The double stranded DNA in any chosen round is sequenced to obtain full-length nanobody sequences (FIG. 1d, Methods). CeVICA then groups the sequences into clusters based on similarity of their CDR sequences, such that each cluster represents a unique binding family (FIG. 1e, Methods). Finally, one representative sequence from each cluster is synthesized and characterized for specific downstream applications (FIG. 1f, Methods). The combination of linear DNA libraries (FIG. 1a), ribosome display (FIG. 1b) and selection cycles (FIG. 1c) allow display of libraries with much larger diversity (>10¹⁰) than methods depending on cells¹⁵ at similar experimental scale. As selection increases the representation of sequences encoding binders, each binder sequence leads to a cluster of sequences in the output library. Computational clustering following high throughput sequencing identifies them efficiently, promising a more comprehensive view of the landscape of binder potential. as compared to methods that rely on the analysis of individual colonies or sequences (Huo et al., 2020; McMahon et al., 2018).

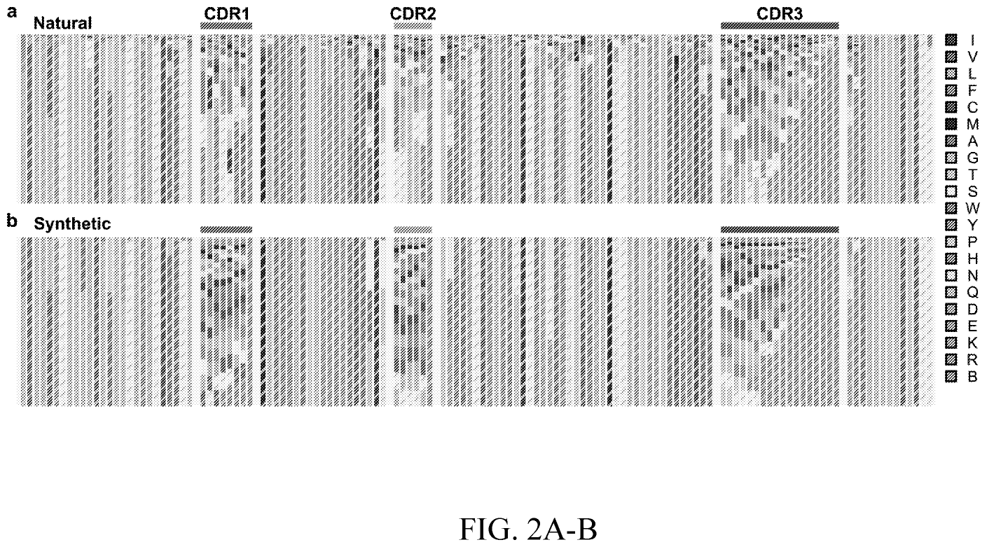

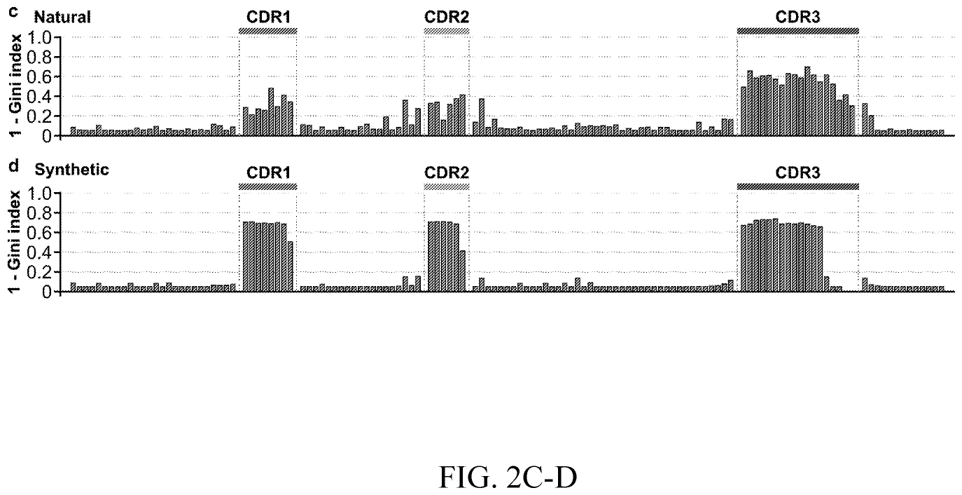

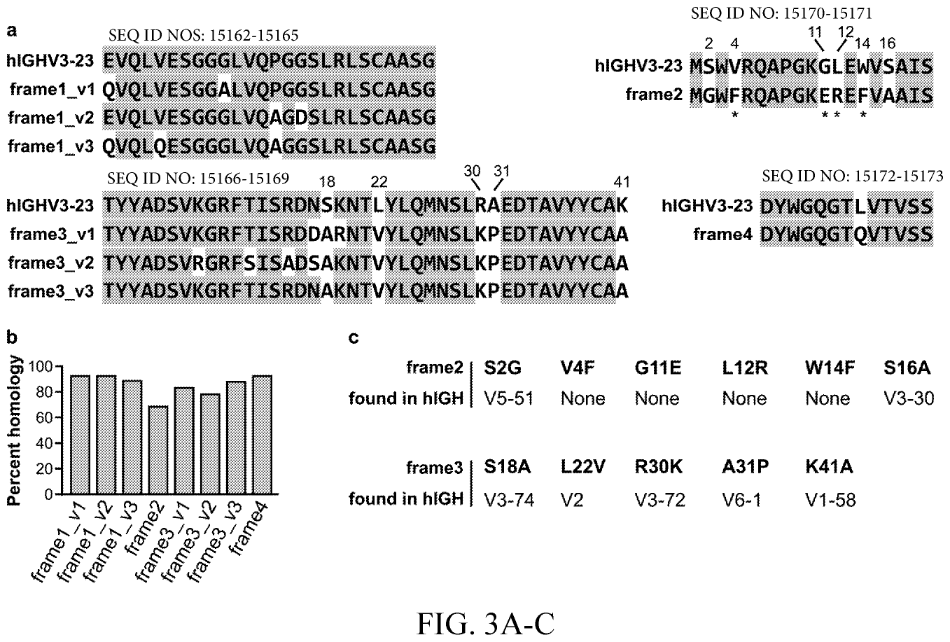

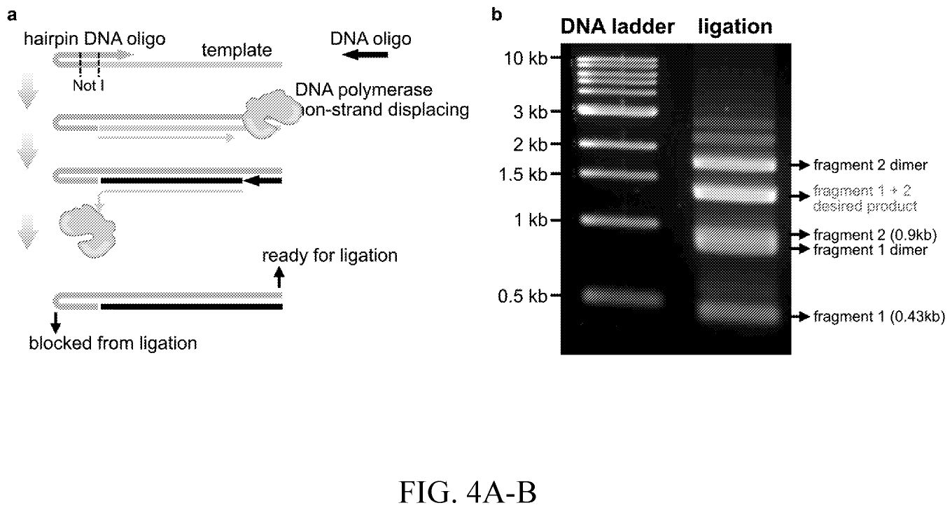

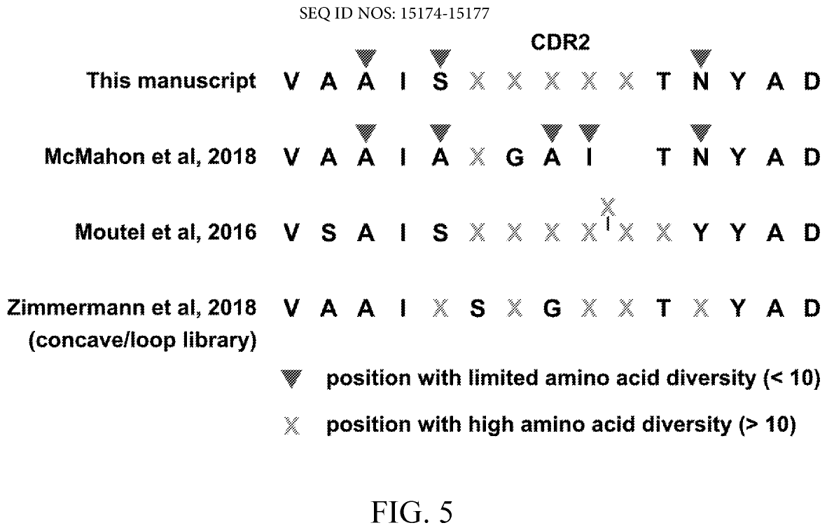

Applicants made VHH libraries containing highly random CDRs, based on analysis of natural VHH sequences and using a three-stage PCR and ligation process (FIG. 1g). First, to guide the VHH library sequence design, Applicants analyzed the sequence characteristics of 298 unique camelid VHHs (representing natural VHHs) from the Protein Data Bank (PDB298) (Table 10A, Methods), highlighting three CDR regions, CDR1-3⁹, separated by four regions of low diversity, frame1-4 (analysis of a larger dataset containing 1,030 sequences from abYsis showed the same sequence features (FIG. 2a, Table 10B). The four frames share high homology with human IGHV3-23 or IGHJ4 (FIG. 3a,b), and most of the remaining non-identical residues are present in other human IGHV genes (FIG. 3c). Applicants used consensus sequences extracted from this profile to design VHH DNA templates encoding the four frames (FIG. 1g), and included additional frames to the final mixture of frame templates (Methods), based on well-characterized nanobodies^{10,16}. The mixture of VHH frames serves as templates in PCR reactions, where DNA oligonucleotides with a 5′ NNB sequence were used to introduce randomization in CDRs, while hairpin DNA oligonucleotides were used to block ligation of one end of the PCR product (FIG. 1g and FIG. 4, Methods). Applicants introduced 7 random amino acids for CDR1, 5 for CDR2, and 6, 9, 10 or 13 for CDR3 to match the most commonly observed CDR lengths in natural VHHs. CDR3s longer than 13 amino acids only account for a minority of natural VHHs (36%, FIG. 2a, Table 11) and were not included in the VHH library. CDRs randomized in earlier stages are subject to duplication in later stages that reduces their diversity. Applicants thus chose to randomize CDR2 first, followed by CDR1, and then CDR3, imposing a diversity hierarchy of CDR3>CDR1>CDR2, because this is the overall ranking of diversity Applicants observed in CDRs in natural VHHs (FIG. 2a,c). The sequence profile of the resulting randomized VHH library met the design objectives, and largely mirrored the sequence features of natural VHHs (FIG. 2 and Table 11). Notably, the library design differs from pervious synthetic nanobody library designs⁶⁻⁸ in several key ways: Applicants defined CDR boundaries and length differently (based on the analysis of natural nanobodies (Table 11, methods), for example, in CDR2 (FIG. 5), and Applicants performed complete randomization of all CDR positions with NNB codons (and do not avoid, for example, cysteines in these positions) to maximize amino acid sequence possibilities. Finally, the VHH DNA library contains an upstream T7 promoter to allow transcription of VHH RNA, a 3×Myc tag, and a spacer downstream of the VHH coding region that stalls peptide release, to enable ribosome display (FIG. 1h).

To test the performance of the library in ribosome display, and to reduce unproductive sequences, such as VHHs that contain frame shifts or early stops, Applicants ribosome displayed a library only with randomized CDR1 and CDR2 and performed one round of anti-Myc selection. Functional VHH sequences will express Myc tag at the C-terminal of VHH and are expected to be enriched after anti-Myc selection. Indeed, there was a large decrease of unproductive sequences and an increase of full-length VHHs (from 25.3% to 51.9%) after anti-Myc enrichment (FIG. 1i). At the DNA level, there was an increase of all in-frame CDR1 DNA lengths and decrease of frame-shift lengths (FIG. 1j, arrows). Applicants used the resulting full-length enriched CDR1 and 2 randomized library as PCR template for randomization of CDR3. The final library with all three CDRs randomized (hereafter, “the input library”) contained 27.5% full-length sequences, and 3.68×10¹¹ full-length diversity per μg of library DNA.

### Binder Selection for RBD and EGFP

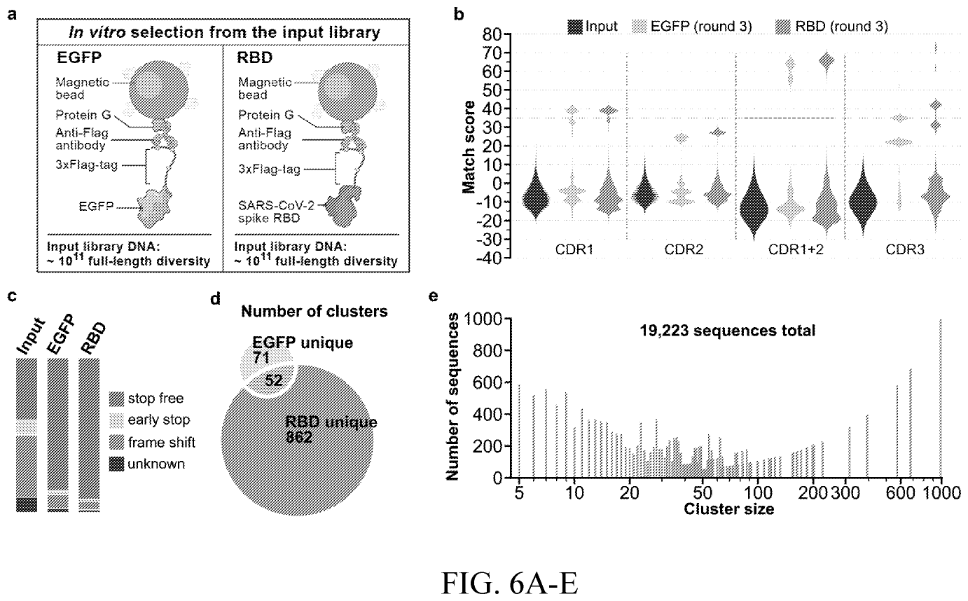

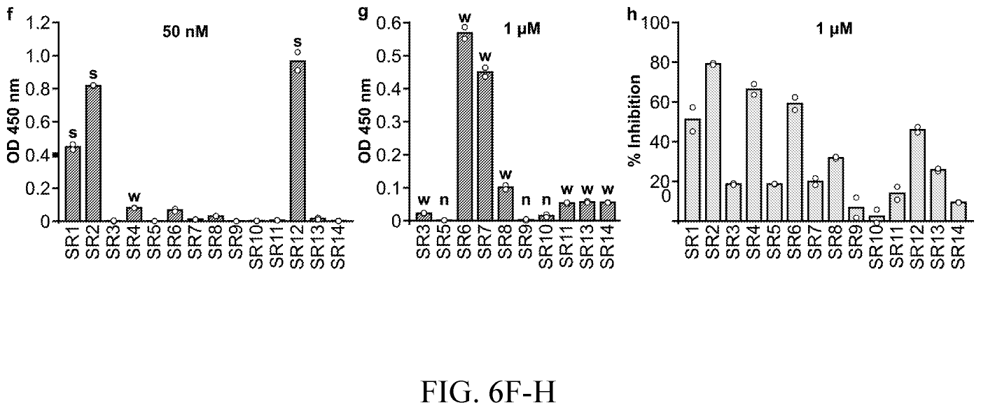

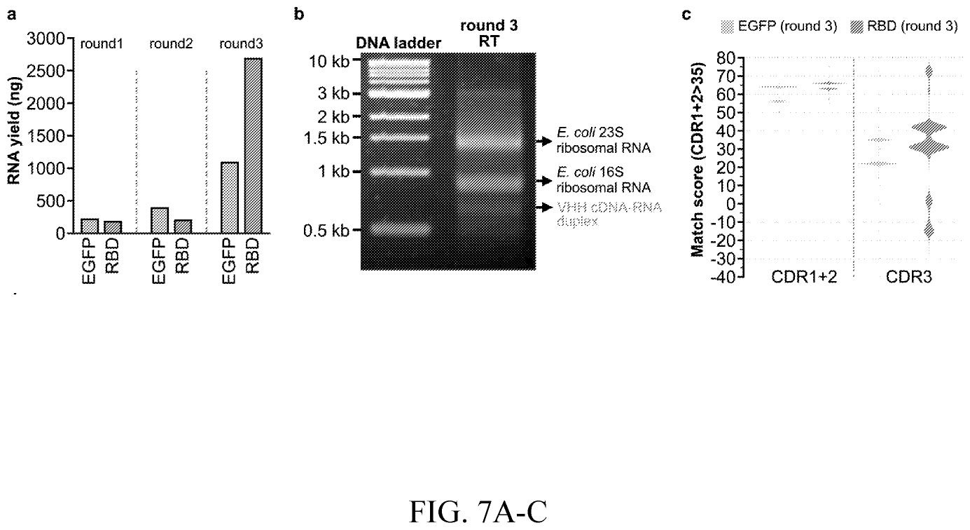

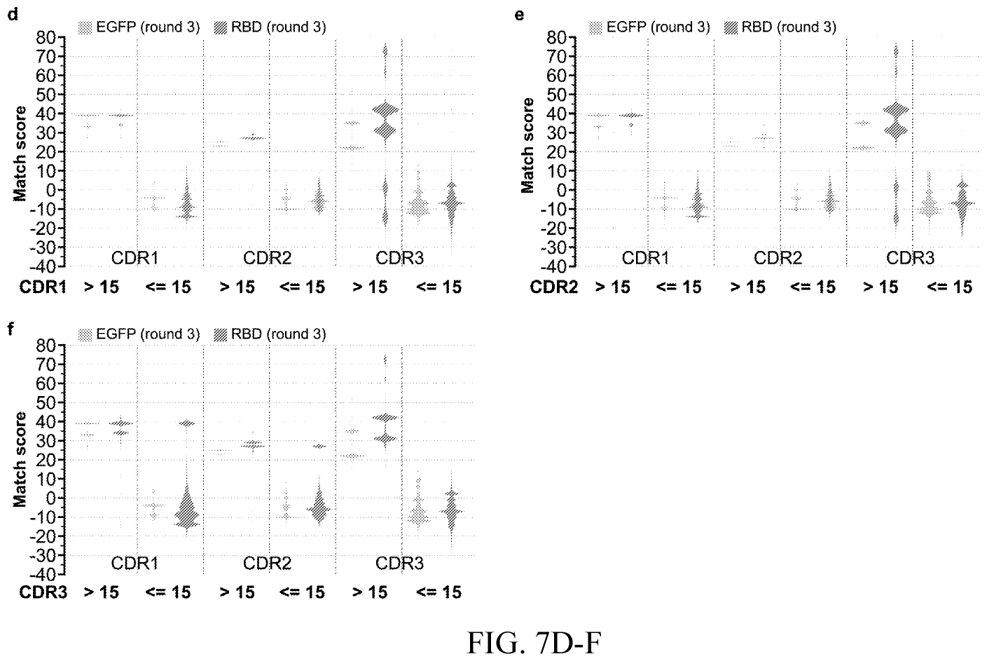

Applicants performed in vitro selection from the input library for sequences that encode binders to two target proteins: EGFP and the receptor binding domain (RBD) of the spike protein of SARS-CoV-2¹⁷ (FIG. 6). Applicants fused each of the two proteins with a 3×Flag tag and immobilized them on beads coated with protein G and anti-Flag antibody (FIG. 6a). For each screen, Applicants used input library DNA corresponding to ˜1×10¹¹ full-length diversity, and performed 3 rounds of selection. After round 3, with an optimized PCR approach that minimized loop shuffling¹⁸ (Methods), RNA yield markedly increased in both screens (FIG. 7a) and the recovered sequences were primarily composed of E. coli ribosomal RNAs and VHH library RNA (e.g., FIG. 7b). Comparing the input and output library sequences shows a 2.3-fold increase in the proportion of stop-free VHH sequences after 3 rounds of selection (FIG. 6c), fitting the expectation that successful binding to targets depends on intact VHH structure.

Applicants identified target specific binders by clustering CDR sequences enriched after selection into families, while accounting for sequencing errors (Methods). First, Applicants examined the distribution of the sequence match scores (Methods) between randomly selected pairs of sequences within a CDR in a library, and compared these distributions for each CDR between the input and output libraries (FIG. 6b, Methods). In the pre-selection input libraries, the mean match score is low and the distribution is unimodal, as expected given the randomization; whereas after selection, there is a multi-modal distribution, with one low mode (similar to input) and at least one high mode (FIG. 6b), which is further distinguished when combining the CDR1 and CDR2 match scores (FIG. 6b). This high mode should reflect binders enriched by the selection rounds. Notably, sequences with a high match score in one CDR are more likely to have a higher match score in other CDRs (FIG. 7c-f). Applicants clustered the likely binder sequences exceeding a combined (CDR1+2) match score threshold (FIG. 6b, dashed horizontal line), yielding 862 unique clusters for RBD and 71 for EGFP, with 52 clusters shared by the two targets (FIG. 6d, Table 7 and 8). The shared clusters represent background binders and are excluded from further analysis due to them not showing specific binding to either EGFP or RBD. Notably, RBD unique clusters span a wide range of cluster sizes (FIG. 6e). Conversely, the shared clusters represent background binders and are excluded from further analysis, because they do not show specific binding to either EGFP or RBD.

Focusing on RBD binders, Applicants chose one representative VHH gene from each of the 14 top-ranking (ranked by cluster size) RBD unique clusters and validated it for spike RBD binding and SARS-CoV-2 pseudovirus neutralization (FIG. 6f-h, Methods). RBD binding ELISA assays of the 14 tested VHHs (SR1-14) showed 3 strong binders (SRL 2, 12), 8 weak binders (SR3, 4, 6, 7, 8, 11, 13, 14) and 3 non-binders (FIG. 6f,g). SARS-CoV-2 S pseudotyped lentivirus neutralization assays revealed 6 VHHs inhibiting infection above 30% at 1 μM (FIG. 6h), which included the 3 strong binders and three of the weak binders (SR4, 6, 8).

### Validation of NNB Codon Fitness for Binder Selection

Applicants next compared input, output and natural CDR sequence distributions to assess whether starting with a fully random CDR amino acid profile could be generally detrimental to the fitness of binders, and whether selection output mimics a natural amino acid distribution. In natural VHHs (PDB298 sequences, table 11), CDR1 and CDR2 are less diverse than CDR3 with an amino acid profile that favors certain residues (FIG. 2a,c), and previous synthetic nanobody library designs sought to recapitulate the CDR1 and CDR2 amino acid preferences of natural nanobodies⁶⁻⁸. Conversely, Applicants used fully-randomized NNB codons to encode all CDR positions. In principle, such a design might not be ideal if the natural CDR1 and CDR2 amino acid profile is required for functional nanobodies; alternatively, it may allow us to recover possibilities not captured by libraries pre-biased by natural sequence distributions.

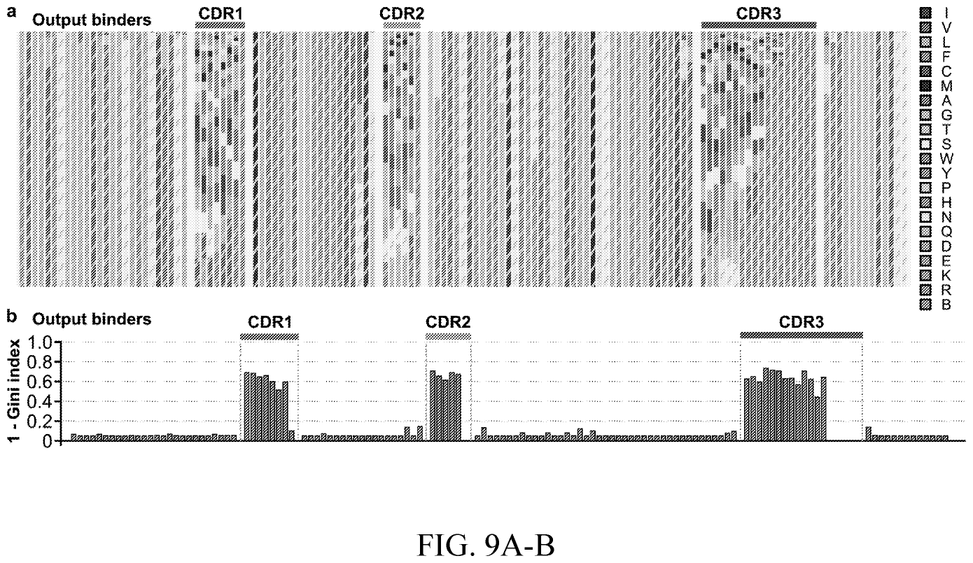

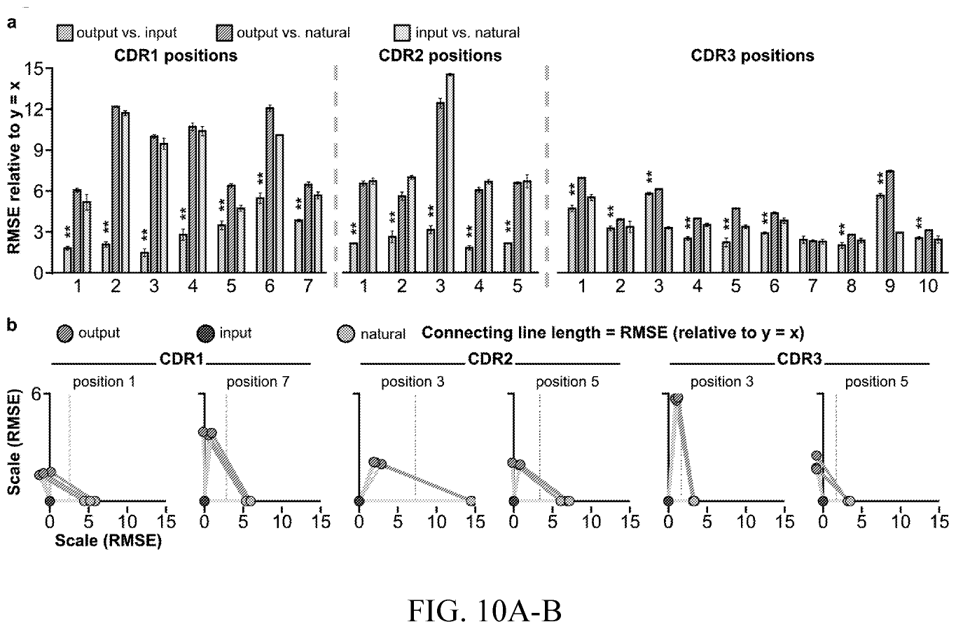

To determine whether the fully random CDR amino acid profile is detrimental to the fitness of binders, Applicants compared the CDR amino acid profile of 932 representative sequences across all unique clusters from both the EGFP and RBD output libraries (“output binders”) (FIG. 9, table 11) to the sequence profiles of either the input library or natural VHHs (FIG. 2a,b). Applicants reasoned that if the amino acid profile in the input library leads to a distribution of proteins that are less fit in binding, the binder selection process should shift this distribution to a more fit profile in the output library, such that there is a low correlation between the amino acid profiles of the input library and output binders. Surprisingly, there was an overall smaller shift in CDR1 and CDR2 compared to CDR3, as indicated by higher Spearman correlation coefficients (FIG. 8a-c, mean Spearman correlation=0.73, 0.73, and 0.64 respectively), and shorter distances (as the RMSE relative to y=x line, Methods, FIG. 8d,e, mean RMSE=2.96, 2.40 and 3.51 respectively), implying that a fully random profile at CDR1 and CDR2 may not have had a substantial binding fitness cost at most positions, whereas CDR3 not only shifted away from the input profile, it was even further shifted from the natural profile (FIG. 8d,e). Moreover, correlation of amino acid profiles between output binders and natural VHHs are significantly less than between output binders and input library at most CDR positions (FIG. 8). A few positions (CDR1 position 7 and CDR3 position 1-3) had much lower input-output binders Spearman correlation coefficients and higher RMSE than most positions. This suggests that these positions may benefit from specifically-designed amino acid profiles (to adjust off diagonal amino acids percentages (FIG. 8b) accordingly), even though their input distributions were not particularly distinct from the natural sequence distribution compared to other positions (FIG. 8a,d). Applicants observed similar results when they used a larger collection of 1,030 natural nanobody collection from abYsis (www.abysis.org, abYsis1030) to calculate the natural profile (FIG. 10). Thus, the output binder CDR profile is predominantly influenced by the input library rather than by selection towards a natural VHH profile, a natural VHH CDR amino acid profile is not required for VHH binding properties, and a fully random CDR design offers high diversity without a major binding fitness cost (although may have other fitness drawbacks in vivo).

### Affinity Maturation Effectively Improves VHH Function

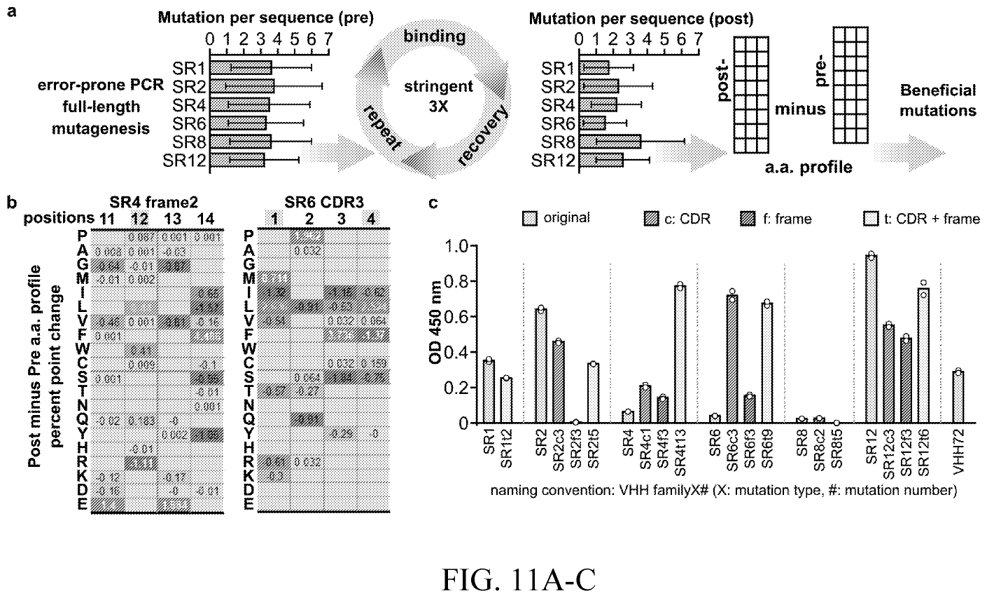

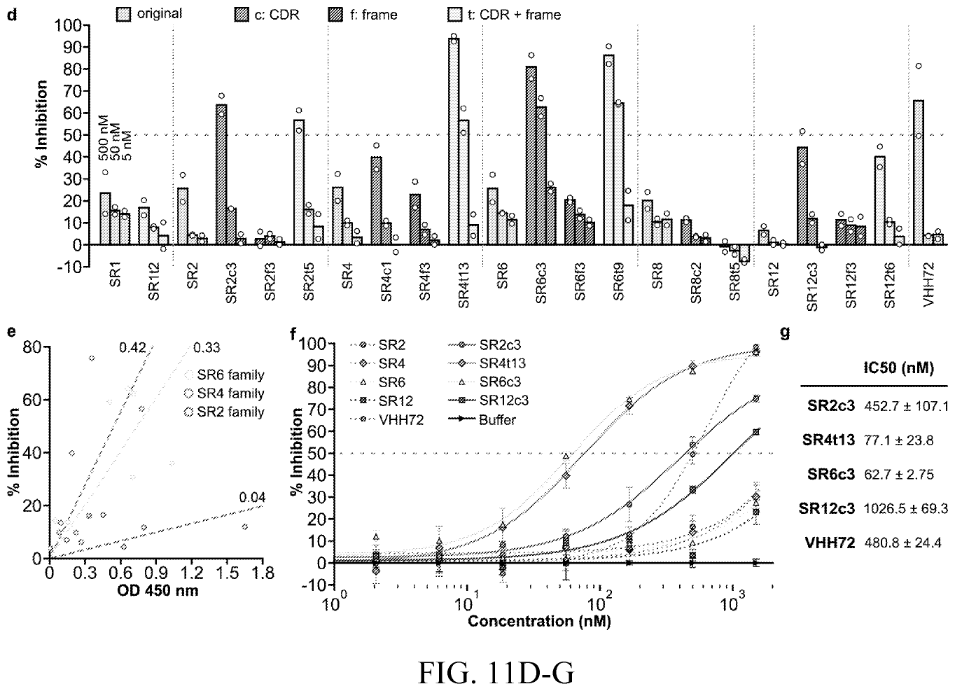

To perform affinity maturation, a critical stage in antibody development in animals, Applicants designed and performed an affinity maturation strategy based on CeVICA to increase the affinity of RBD binding VHHs (FIG. 11a, Methods). Applicants used error-prone PCR to introduce random mutations across the full-length sequence of six selected VHHs (SR1, 2, 4, 6, 8, 12) and generated the mutagenized library. A library size of 4.18×10¹⁰ diversity (sufficient to cover the full diversity of VHHs with three mutations per sequence) was used as input and three rounds of stringent selection were performed. Applicants sequenced the libraries pre- and post-affinity maturation, and observed about 3 mutations in the pre-library and about 2 mutations in the post-library per sequence (FIG. 11a). Applicants calculated their position-wise amino acid profiles, and determined, for each VHH, the change in each amino acid proportion at each position, generating a percent point change table. Applicants defined putative beneficial mutations as those with a percent point increase above a set threshold (FIG. 11b, Methods and Table 13), highlighting between 8 to 25 putative beneficial mutations for each of the selected VHHs. Finally, Applicants assembled a list of identified putative beneficial mutations for each VHH and incorporated different combinations of them into each VHH parental sequence to generate multiple mutated variants of each VHH for final assessment (Table 9).

Variants in the SR4 and SR6 families had both increased binding and neutralization, while the SR2 and SR12 family variants had only increased neutralization but not binding, based on an ELISA binding assay and a pseudotyped virus neutralization assay (FIG. 11c,d). Multiple nanobody variants outperformed VHH72, a previously described nanobody that neutralizes SARS-CoV-2 pseudoviruses¹⁹, in binding (e.g., SR12c3), neutralization (e.g., SR4t6), or both (e.g., SR6c3) (FIG. 11c,d and Table 14). Neutralization and binding performance were poorly correlated across variants (r²=0.07), as previously reported²⁰. However, when considering each nanobody family separately, trends were stronger, and neutralization and affinity were more highly correlated for SR4 and SR6 nanobodies (FIG. 11e). This may be because variants within the same family share the same binding site and orientation. One intriguing hypothesis is that the slope of each VHH family's linear trend reflects the sensitivity of the virus to the blocking of the family's binding site. A dose response curve of selected VHHs showed SR6c3 as the most potent neutralizer (FIG. 11f) with an IC50 of 62.7 nM (FIG. 11g), comparable to potent SARS-CoV-2 neutralizing antibody Fab domains²¹ and monoclonal antibodies²² identified from human patients. Importantly, the original SR6 cluster contained only 679 sequences, representing 0.67% of the 101,674 sequences from the initial selection output, highlighting the power of CeVICA in rapidly identifying high performance antibodies among a vast number of potential candidates.

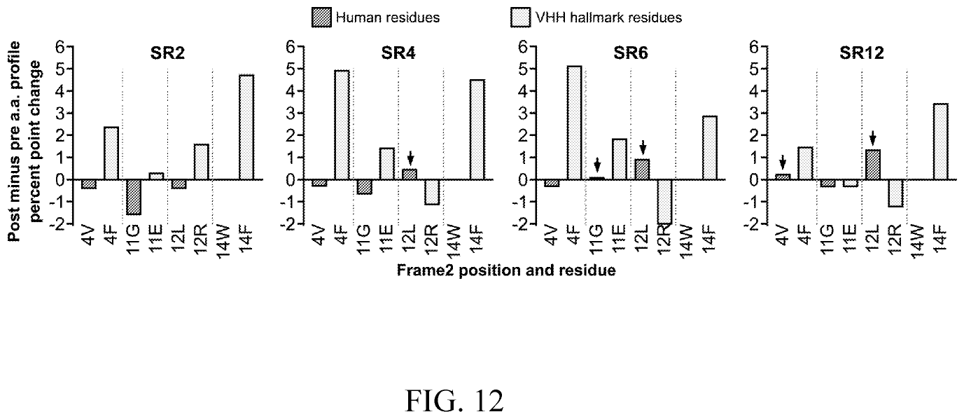

Next, Applicants examined the potential impact that the VHH sequences may have on immunogenicity in humans, as a major concern related to the therapeutic use of VHH antibodies is the possibility that, as camelid proteins, they would elicit an immune response. In particular, VHH hallmark residues in frame2 constitute a major difference between camelid VHHs and human VHs (FIG. 3). Applicants used the affinity maturation data to identify potential conversion options for these VHH hallmark residues. In three of the four VHH hallmark residues Applicants found VHHs where the residues were converted to the corresponding human residue as a result of affinity maturation (FIG. 12, arrows). These data imply that at least some of the VHH hallmark residues can be converted to human residues without loss of binding fitness. Such conversions may serve as frame features of future VHH library designs and improve tolerance of in vitro engineered VHHs by humans. Notably, single domain antibody frames containing all four human hallmark residues have been successfully used for in vitro engineering of single domain antibodies without light chain²³, demonstrating the feasibility of converting VHH hallmark residues to human residues. Overall, the extension of CeVICA for affinity maturation offers a strategy for improving antibody function.

### True Binders and Neutralizers Identified Across the CeVICA Predicted List.

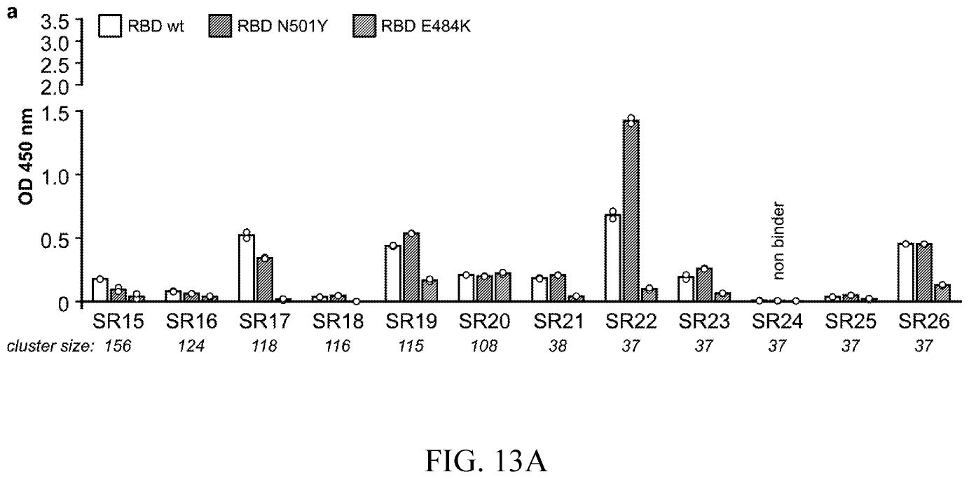

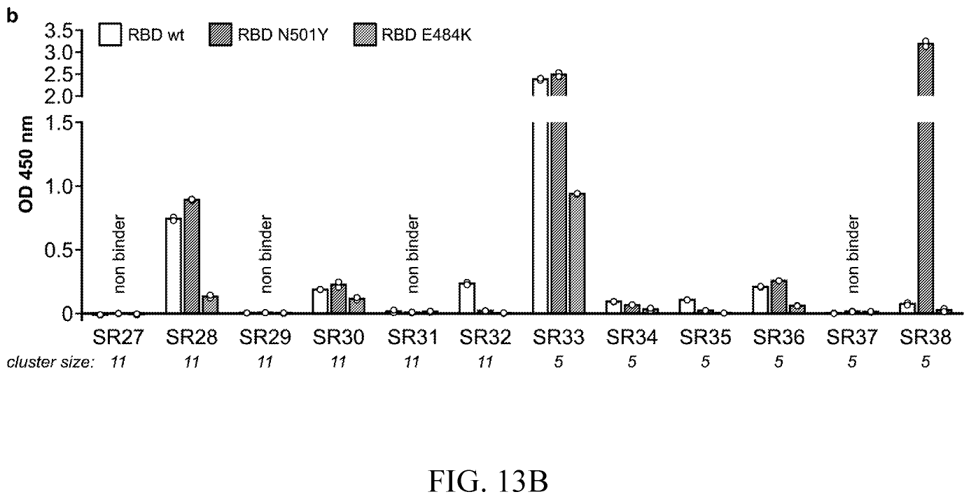

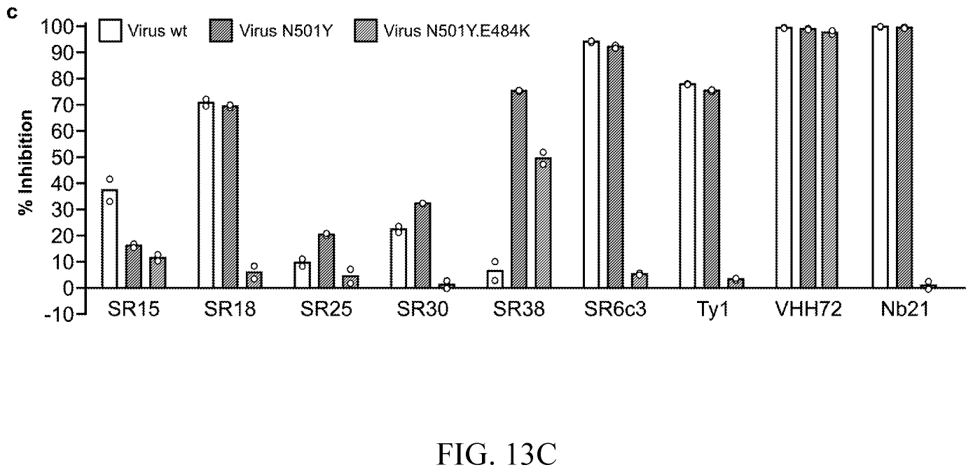

Applicants next asked whether true binders and/or neutralizers can be identified from lower ranked clusters across the full list of 862 clusters. To this end, Applicants cloned and purified 24 additional VHHs representing 24 clusters selected from various locations on the list of clusters ranked by cluster size (SR15-38, with their respective original cluster size ranging from 156 to 5, FIG. 13). These VHHs were assayed by both ELISA and pseudovirus neutralization against wild type RBD/spike along with the recently emerged RBD/spike variants N501Y and E484K²⁴. 19 VHHs showed positive ELISA readings (back ground subtracted OD 450 nm >0.02, FIG. 13a,b) and 5 VHHs (SR15, 18, 25, 30, 38) showed greater than 20% inhibition at 1 μM for at least one RBD/spike variant (FIG. 13c). Notably, SR38, representing a cluster with cluster size of 5 that ranked at the bottom of the list of 862 clusters, binds N501Y RBD strongly and showed stronger inhibition of pseudoviruses carrying N501Y and E484K mutations compared to two previously identified nanobodies of animal origin, Ty125 and Nb2126 (FIG. 13c). Taken together, Applicants identified 30 positive binders among a total of 38 tested VHHs (78.9% positive rate), further validating the efficacy of the CDR-directed clustering approach for selection of binders.

### Engineering Potent and Stable VHHs for Virus Neutralization.

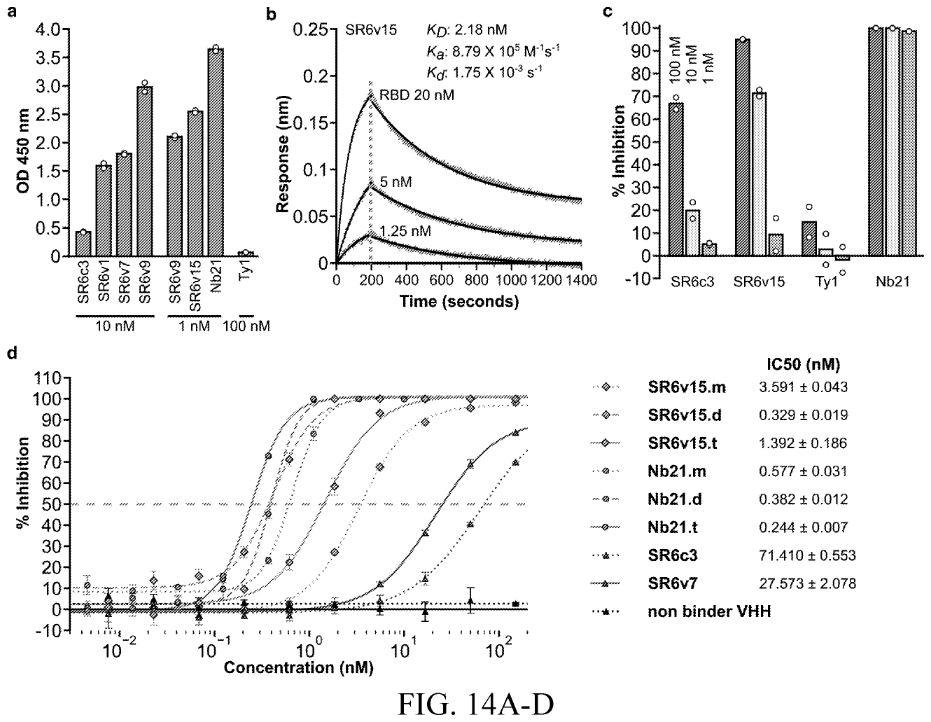

To engineer more potent virus neutralizing agent, Applicants performed a second affinity maturation using SR6c3 as the baseline template. Applicants identified mutation combinations that greatly enhanced binding affinity (SR6v1, SR6v7, SR6v9 and SR6v15) compared to SR6c3 (FIG. 14a, Table 9 and 14). SR6v15, the variant with the highest binding shown by ELISA, has a K_{D} of 2.18 nM as measured by biolayer interferometry (FIG. 14b) and inhibited pseudovirus infection more potently than SR6c3 (FIG. 14c). Applicants further converted SR6v15 (SR6v15.m) into tandemly linked dimer (SR6v15.d) and trimer (SR6v15.t), and compared them to Nb21′ based agents (monomer: Nb21.m, dimer: Nb21.d, trimer: Nb21.t) in pseudovirus neutralization assay (FIG. 14d). The most potent SR6v15 based agent, SR6v15.d had a IC50 of 0.329 nM, while the most potent Nb21 based agent, Nb21.t, had a IC50 of 0.244 nM (FIG. 14d). These results demonstrate CeVICA's capability to produce highly potent virus neutralizing agents through iterative optimization.

CeVICA Selected Nanobodies have Desirable Biophysical Characteristics and are Stable

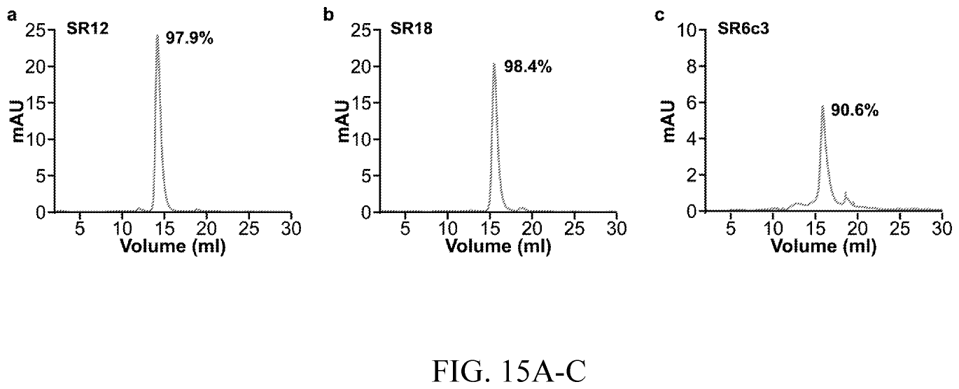

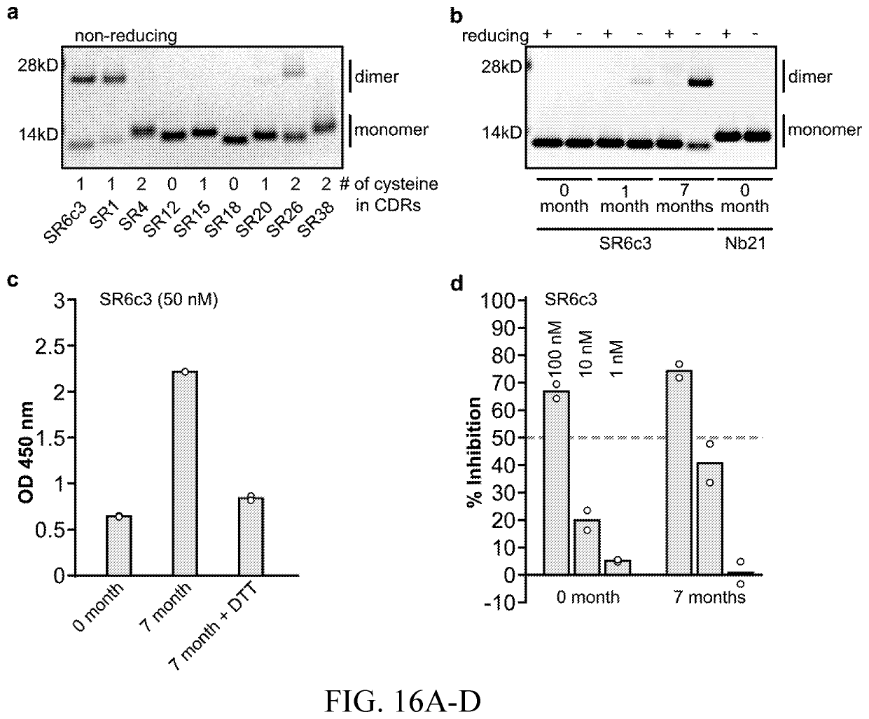

CeVICA uses NNB codons to randomize CDRs, which may cause over-representation of certain amino acids that could contribute to poor biophysical properties in the output VHHs. Applicants evaluated the extent of such potential undesired effects by several biophysical assays. First, Applicants performed size exclusion chromatography analysis of three nanobodies (SR12, SR18, SR6c3) and found that for each of them >90% of the molecules exist as monomers (FIG. 15). Second, Applicants investigated the impact of cysteines in CDRs on nanobody biophysical properties and function because cysteine occurs at much higher frequencies in the library CDRs (5.8% in input library and 6.0% in unique output binders (Table 11) than in natural VHH CDRs (averaging 2.1% in CDR3 positions 7-12, Table 11. Non-reducing SDS-PAGE gel analysis of VHHs with 0-2 cysteines in their CDRs (using samples stored at 4° C. for at least 4 weeks) revealed that VHHs with no CDR cysteine (SR12, SR18) only had one monomer band (FIG. 16a), while VHHs with 1 or 2 CDR cysteine either had a single monomer band (SR4, SR15, SR38) or a monomer and a dimer band (SR6c3, SRL SR20, SR26) (FIG. 16a). Using SR6c3 samples that has been stored for varying lengths of time, Applicants found dimers were not detected in freshly purified samples and appeared overtime at relatively low rate (FIG. 16b). Thus, presence of cysteine in CDRs do not always cause VHH dimers due to disulfide bond formation. Applicants next evaluated the functional consequences of CDR cysteine mediated dimer formation. 7 months old SR6c3 sample showed increased signal in ELISA compared to fresh sample and the signal increase is suppressed by treating with reducing reagent that breaks up disulfide bond (FIG. 16c). 7 months old SR6c3 sample also inhibited pseudovirus infection greater than fresh sample (FIG. 16d), consistent with ELISA data and indicates that disulfide bond formation via CDR cysteine does not adversely affect the function of SR6c3.

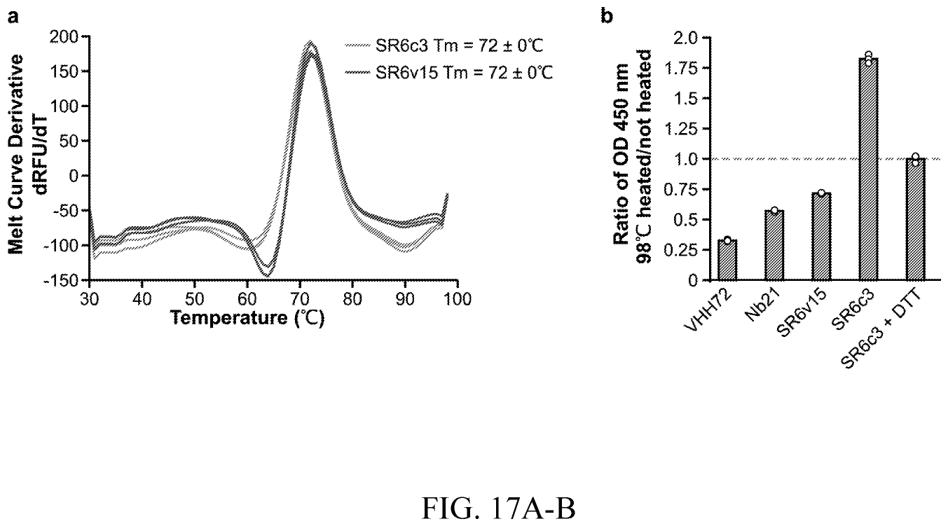

Finally, Applicants assessed the thermal stability of VHHs produced by CeVICA. Both SR6c3 and SR6v15 showed good resistance to thermal denaturation and had a melting temperature of 72° C. (FIG. 17a), which is comparable to VHHs generated by other methods 23. Applicants then tested different VHH's ability to refold after complete thermal denaturation by comparing ELISA readings of VHH samples before and after heating at 98° C. for 10 minutes. SR6v15 showed higher refolding with a heated/non heated ratio of 0.72 compared to that of VHH7219 (0.33) and Nb2126 (0.57) (FIG. 17b). Surprisingly, SR6c3 had a heated/non heated ratio greater than 1, indicating increased binding affinity after complete thermal denaturation and refolding. Applicants hypothesized that this increase may result from expedited disulfide bond formation that increased the percentage of dimers in SR6c3 samples subjected to heating. This hypothesis is supported by the observation that SR6c3 samples heated and refolded in the presence of reducing reagent had a heated/non heated ratio of 1 (FIG. 17b). Thus, VHHs produced by CeVICA has good thermal stability and can efficiently refold after complete thermal denaturation.

### DISCUSSION

The CeVICA platform offers a generalizable solution for in vitro VHH antibody engineering that integrates all the components necessary to generate VHH binder sequences in a cell-free process (FIG. 1a). CeVICA VHH library is designed to contain only the essential features for robust VHH structure, revealed by the diversity profile across the length of natural VHHs (FIG. 2a,c). Importantly, Applicants validated that fully random NNB encoded codons in all CDR positions does not adversely affect binder selection (FIG. 8) nor does it impact biophysical stability of individual VHHs produced by the platform (FIG. 15, FIG. 16, FIG. 17). A linear DNA library of such design can be efficiently produced by a method of successive PCR and ligation (FIG. 1g), yielding large libraries with their library size directly quantifiable. This library generation method is highly adaptable, for example, oligos containing alternative base mix ratios can be used to achieve different amino acid profiles for specific CDR positions, alternative frame template sequences can be used to enrich for unique biophysical properties encoded in the frame regions of VHHs. Lastly, these linear libraries perform well when used as input to an optimized ribosome display based selection protocol, which suppresses sequence segment shuffling that could break up CDR pairing (Methods), a challenging problem often associated with cell free systems¹⁸.

A key feature of CeVICA is binder sequence recover using CDR-directed clustering. This approach fully utilizes all sequences in the output library to provide a comprehensive view of all binders contained in the output (FIG. 1d-f, FIG. 6b-e) and, in effect, reduces the VHH characterization screen space (e.g., From 19,223 VHH sequences in RBD output library to 862 in the list of VHH clusters) (FIG. 6b,d,e). This feature makes CeVICA particularly suited for applications where large numbers of antibodies need to be screened to isolate ones with unique traits (e.g., virus neutralization, receptor activation, targeting hard-to-target epitopes) in addition to binding the target. Indeed, when Applicants applied CeVICA to engineer SARS-CoV-2 neutralizing VHHs, Applicants were able to identify SR38, a VHH with a rare ability to strongly favor binding of N501Y containing RBD and neutralizes N501Y containing pseudovirus more potently than pseudovirus not carrying N501Y (FIG. 13), making SR38 a potential candidate for the development of N501Y variant specific detection reagents and cross-variant neutralization agents. Importantly, SR38's cluster only contain 5 sequences, representing ˜0.03% of total, making it difficult to recover by random sampling without computational clustering.

Previous synthetic nanobody library designs sought to randomize CDR positions using an amino acid profile that recapitulates the profile observed in the corresponding positions in natural nanobodies; however, to the best of Applicants' knowledge, whether the natural profile represents an ideal profile for the purpose of in vitro antibody engineering has not been thoroughly investigated experimentally. The large number of nanobody clusters Applicants generated using CDR-directed clustering offered the opportunity to test the fitness of randomized amino acid profile in binder selection (FIG. 8). Applicants found that in many positions, the output binder profile highly resembles the input library profile, while the similarity between the output profile and the natural profile is lower. For positions where the output profile moved significantly away from the input profile (e.g., CDR1 position 7), the distance between output and natural profiles is greater than that between output and input, and also greater than the distance between input and natural, indicating that the output profile is not moving closer to a natural profile in these positions (FIG. 8d,e). Thus, Applicants did not find evidence indicating that the amino acid profiles observed in natural nanobodies are more fit than an NNB profile for binder selection (although they may be more fit for other features). These data also suggest a strategy to improve the fitness of an input library by incorporating amino acid profiles that matches the output profile, which can be achieved by using specifically defined (non-equal) base mix ratios for the three base positions of a randomizing codon. Such a strategy could provide future improvements on synthetic nanobody library design.

VHHs produced by CeVICA showed good biophysical properties that are comparable to VHHs of animal origin (FIG. 15, FIG. 16, FIG. S17). Notably, Applicants saw robust refolding after complete thermal denaturation up to 100% (SR6c3) (FIG. 17b). Such high refolding capability may be partly explained by the use of ribosome display for the selection of these VHHs, during which VHHs need to fold into their functional confirmation while tethered to ribosomes in a minimally reconstituted protein synthesis environment that lacks factors normally found inside cells to aid protein folding such as chaperons, thus enriching for VHHs with strong inherent folding stability. This hypothesis could be tested further as CeVICA gets applied to more cases. The most potent nanobody generated in this study, SR6v15, outperformed two of the previously reported nanobodies generated through animal immunization, Ty125 and VHH7219, in both the binding affinity to RBD and the potency of pseudovirus infection (FIG. 14a,c). A dimeric form of SR6v15, SR6v15.d, had a more than 10-fold increase in pseudovirus neutralization potency compared to the monomeric SR6v15. SR6v15.d's IC50 is comparable to that of Nb21.t, a previously reported nanobody with high virus neutralization potency²⁶ (FIG. 14d). Together, these data demonstrate CeVICA's suitability for engineering high affinity VHH antibodies with comparable biophysical properties as VHHs produced by animals, making it a valuable addition to in vitro antibody engineering technologies.

In conclusion, CeVICA is a new system for synthetic VHH based antibody library design, in vitro selection optimization, post-selection screening, and affinity maturation. Using CeVICA, Applicants generated a large collection of antibodies that can bind the RBD domain of the SARS-CoV-2 spike protein and can neutralize pseudotyped virus infection, thus providing an important resource. Given its seamlessly integrated procedure, CeVICA is amenable to automation and could provide an important tool for antibody generation in a rapid, reliable and scalable manner. CeVICA further provides a technology framework for incorporation of future refinements that could overcome limitations of in vivo fitness of in vitro generated antibodies and the overall efficiency of cell-free antibody engineering.

### Example 2—Materials and Methods

Constructs. DNA encoding VHHs were obtained by gene synthesis (IDT) and cloned into pET vector in frame with a C-terminal 6×His tag or GST tag by Gibson assembly (NEBuilder® HiFi DNA Assembly Master Mix, New England Biolabs). DNA encoding SARS-CoV-2 S RBD (S a.a. 319-541) were obtained by gene synthesis and cloned into pcDNA3 with an N-terminal SARS-CoV-2 S signal peptide (S a.a. 1-16) and a C-terminal 3×Flag tag by Gibson assembly. EGFP was cloned into pcDNA3 with a C-terminal 3×Flag tag by Gibson assembly. SARS-CoV-2 S was amplified by PCR (Q5 High-Fidelity 2× Master Mix, New England Biolabs) from pUC57-nCoV-S(kind gift from Jonathan Abraham lab). SARS-CoV-2 S was deleted of the 27 a.a. at the C-terminal and fused to the NRVRQGYS sequence of HIV-1, a strategy previously described for retroviruses pseudotyped with SARS-CoV S²⁵. Truncated SARS-CoV-2 S fused to gp41 was cloned into pCMV by Gibson assembly to obtain pCMV-SARS2ΔC-gp41. psPAX2 and pCMV-VSV-G were previously described²⁶. pTRIP-SFFV-EGFP-NLS was previously described²⁷ (a gift from Nicolas Manel; Addgene plasmid #86677; http://n2t.net/addgene:86677; RRID:Addgene_86677). cDNA for human TMPRSS2 and Hygromycin resistance gene was obtained by synthesis (IDT). pTRIP-SFFV-Hygro-2A-TMPRSS2 was obtained by Gibson assembly.

Cell culture. HEK293T cells were cultured in DMEM, 10% FBS (ThermoFisher Scientific), PenStrep (ThermoFisher Scientific). HEK293T ACE2 were a kind gift of Michael Farzan. HEK293T ACE2 cells were transduced with pTRIP-SFFV-Hygro-TMPRSS2 to obtain HEK293T ACE2/TMPRSS2 cells. The transduced cells were selected with 320 μg/ml of Hygromycin (Invivogen) and used as a target in SARS-CoV-2 S pseudotyped lentivirus neutralization assays. Transient transfection of HEK293T cells was performed using TransIT®-293 Transfection Reagent (Minis Bio, MIR 2700).

Amino acid profile construction and analysis of natural VHHs. VHH protein sequences were downloaded from the Protein Data Bank (only entries deposited prior to Sep. 2^{nd}, 2020 were included; Table 10) or abYsis (www.abysis.org/abysis, date 2021-05-01, Table 10). Nanobodies (VHHs) were separated into CDRs and frames (segments) by finding regions of continuous sequence in each VHH that best matched to the following standard frame sequences:

frame1 standard:
(SEQ ID NO: 15158)
EVQLVESGGGLVQAGDSLRLSCTASG,
frame2 standard:
(SEQ ID NO: 15159)
MGWFRQAPGKEREFVAAIS,
frame3 standard:
(SEQ ID NOS: 15160)
AFYADSVRGRFSISADSAKNTVYLQMNSLKPEDTAVYYCAA,
frame4 standard:
(SEQ ID NOS: 15161)
DYWGQGTQVTVSS,

Each matched region is the corresponding frame of the VHH, the region between frame1 and frame2 is CDR1, the region between frame2 and frame3 is CDR2, the region between frame3 and frame4 is CDR3 (FIG. 1g). Only nanobody sequences with at least one unique CDR were selected to represent natural nanobodies and used for constructing amino acid profile (a.a. profile). 298 sequences from Protein Data Bank (PDB298) and 1,030 sequences from abYsis (abYsis1030) fit this selection criteria (Table 10). The amino acid (a.a.) profile at each position within each segment was calculated by finding the percentage of each of the 20 universal proteinogenic amino acid at that position among all selected VHHs, all frame lengths were set to the same length as frame standards. CDR lengths were manually set to accommodate different CDR lengths, CDR1 and CDR2 lengths was set to 10, CDR3 length was set to 30. VHHs with CDR lengths shorter than the corresponding set length had their CDR filled from the C-terminal end with empty position holders up to the set length. Numbers in amino acid profile table are the percentage of each amino acid. CDR boundaries were defined by the position where the combined frequency of the top two most abundant amino acids dropped sharply.

Applicants compared their annotation method to Kabat and Chothia annotation (www.abysis.org/abysis/sequence_input/key_annotation/key_annotation.cgi) and found all three methods (Kabat, Chothia and Applicants) showed frame regions with the same core sequence, and with 1-2 amino acid differences in the exact CDR boundaries between the three methods. The performance of Applicants' library suggests their annotation faithfully captured the domain structure of nanobodies.

Applicants used 1—Gini index to measure the level of diversity at each amino acid position. The Gini index measures the degree of inequality among individuals in a population, ranging from 0, when resources are uniformly (equally) distributed across individuals, and 1, when one member has all the resources. Applicants' diversity index of 1-Gini takes 0 when there is no diversity (one amino acid has an abundance of 100%) and 1 for the highest diversity (all amino acids have the same abundance). The diversity index is calculated for 8 positions for CDR1, 6 positions for CDR2 and 18 positions for CDR3 for all sequence groups, when no sequence in the group contain a certain CDR position, the diversity index will be 0. For example, in CDR2, both the natural nanobody collection and Applicants' input library contained a very small percentage of nanobodies having CDR2 with 6 a.a., while the output binder collection has no nanobody having CDR2 with 6 a.a., hence the diversity index has a value of 0 for the output binder plot in FIG. 9b but a non-zero value for natural nanobodies and input library in FIG. 2c,d.

VHH library construction. Nanobody library sequence is designed to recapitulate the sequence structure of frames and CDRs observed from analyzing natural nanobodies (PDB298, abYsis1030, Table 11). The design differs from prior designs⁶⁻⁸ in both the length of CDRs, the positions selected for randomization and randomization strategy. Such differences likely arise from differences in the size of natural nanobody collection retrieved from databases (93 in McMahon et al.⁶ versus 298/1030 in this study) and/or in how the nanobodies are annotated and analyzed (Amino acid profile construction and analysis of natural nanobodies). For example, the analysis showed the percentage of nanobodies containing CDR2 with lengths 4, 5, or 6 amino acids (a.a). are 32%, 61%, and 1.7% respectively, Applicants thus chose to use CDR2 with a length of 5 a.a. to recapitulate the most prevalent CDR2 length. In contrast, McMahon et al.₆ used an equivalent CDR2 length of 4 a.a., while Moutel et al.⁷ used an equivalent length of 6 a.a. (FIG. 5).

VHH libraries were constructed by ligation of PCR products in three stages, with each stage randomizing one of the three CDRs. Primers used and PCR cycling conditions for each primer pair are listed in Table 12. Primers were synthesized by IDT (www.idtdna.com) using the standard DNA oligo synthesis and purified by desalting without PAGE purification, Applicants find the level of synthesis errors with standard oligo synthesis and desalting purification do not have significant impact on the functionality of the nanobody library. At each stage, PCR was performed using a high-fidelity DNA polymerase without strand displacement activity, using Phusion DNA polymerase (New England Biolabs, M0530L). Importantly, 65° C. was used as the elongation temperature to avoid hairpin opening during DNA elongation. PCR products with correct size were purified by DNA agarose gel extraction. Ligation and phosphorylation of PCR products were performed simultaneously using T4 DNA ligase (New England Biolabs, M0202L) and T4 Polynucleotide Kinase (New England Biolabs, M0201L). Ligation products with the correct size were purified by DNA agarose gel extraction using NucleoSpin Gel and PCR Clean-Up Kit (Takara, 740609.250, this kit was used for all DNA agarose gel extraction steps in this study). Purified ligation products were quantified with Qubit 1× dsDNA HS Assay Kit (ThermoFisher Scientific, Q33230, this kit was used for all Qubit measurements in this study) using Qubit 3 Fluorometer.

CDR2 was randomized in stage one, PCR templates at this stage were equal molar mixtures of plasmids carrying DNA encoding frames, including three frame1 versions, one frame2, three frame3 versions and one frame4. The three versions of frame1 and frame3 were derived from consensus sequence extracted from natural VHH a.a. profile, the A3 VHH′ and a GFP binding VHH¹⁶. Amino acid sequences of the frames are shown in FIG. 2.

CDR1 was randomized in stage two, 200 ng of ligation product from the first stage were digested by NotI-HF (New England Biolabs, R3189S) and heat denatured, the entire digestion product was used as template for PCR in stage two. Ligation product of stage two was subject to one round of ribosome display and anti-Myc selection (below), the entire recovered RNA was reverse transcribed and PCR amplified and purified.

270 ng of this RT-PCR product was used as template for PCR in stage three to randomize CDR3. Ligation product of stage three was purified by DNA agarose gel extraction. The purified ligation product was then digested by DraI (New England Biolabs, R0129S) and a fragment of ˜680 bp in size was purified by DNA agarose gel extraction to get the final VHH library, referred to as the input library.

High throughput full-length sequencing of VHH library. Sequencing libraries from VHH DNA libraries were prepared by two PCR steps using primers and PCR cycling conditions listed in Table 12. Equal mixtures of Phusion DNA polymerase (New England Biolabs, M0530L) and Deep Vent DNA polymerase (New England Biolabs, M0258L) were used for both PCRs to ensure efficient amplification. PCR cycle number was chosen to avoid over-amplification and typically falls between 5 to 15.

In the first PCR, Illumina universal library amplification primer binding sequence and a stretch of variable lengths of random nucleotides were introduced to the 5′ end of library DNA. And similarly, Illumina universal library amplification primer binding sequence and a stretch of variable lengths of index sequence are introduced to the 3′ end of library DNA. Eight different lengths were used for both random nucleotides and index to create staggered VHH sequences in the sequencing library, this arrangement is required for high quality sequencing of single amplicon libraries on an Illumina Miseq instrument. The product of the first PCR was purified by column clean-up using NucleoSpin Gel and PCR Clean-Up Kit and the entire sample was used as template for the second PCR.

In the second PCR, Illumina universal library amplification primers were used to generate sequencing library. Sequencing libraries were purified by DNA agarose gel extraction, quantified using Qubit 3 Fluorometer, and sequenced on an Illumina Miseq instrument using MiSeq Reagent Nano Kit v2 (500-cycles) (Illumina, MS-103-1003), no PhiX control library spike-in was used. Sequencing run setup was: paired end 2×258 with no index read. Index in the library was designed as inline index, so a separate index read was not required. Raw reads are separated by index, trimmed to remove N bases and bases with a quality score of less than 10 prior to downstream analysis.

Ribosome display. VHH DNA library containing a specified amount of diversity was first amplified using a DNA recovery primer pair listed in Table 12. Equal mixtures of Phusion DNA polymerase (New England Biolabs, M0530L) and Deep Vent DNA polymerase (New England Biolabs, M0258L) were used for the PCR. PCR cycle number was chosen to avoid over-amplification and typically falls between 5 and 15. In a standard preparation, 200-500 ng of the purified PCR product was used as DNA template in 25 μl of coupled in vitro transcription and translation reaction using PURExpress In Vitro Protein Synthesis Kit (New England Biolabs, E6800L). The reaction was incubated at 37° C. for 30 minutes, then placed on ice, and 200 μl ice cold stop buffer (10 mM HEPES pH 7.4, 150 mM KCl, 2.5 mM MgCl₂, 0.4 μg/μl BSA (New England Biolabs, B9000S), 0.4 U/μl SUPERase•In (ThermoFisher Scientific, AM2696), 0.05% TritonX-100) was then added to stop the reaction. This stopped ribosome display solution was used for binding to immobilized protein targets during in vitro selection. The amount of DNA template, volume of coupled in vitro transcription and translation reaction, and volume of stop buffer were scaled proportionally when different volumes of stopped ribosome display solution was needed. 1 to 8× standard preparations were used for each selection round with the first round using 8× standard preparations, second round using 2× standard preparations and third round using 1× standard preparation.

In vitro selection. Target proteins were immobilized to magnetic beads by first coating protein G magnetic beads (ThermoFisher Scientific, 10004D) with anti-Flag antibody (Sigma-Aldrich, F1804), then incubating antibody-coated beads with cell lysate or cell media containing 3×Flag tagged target proteins at 4° C. for 2 hours. For anti-Myc selection, magnetic beads were coated by anti-Myc antibody (ThermoFisher Scientific, 13-2500) only. 100 μl of beads was used for the first round of selection, and 50 μl of beads was used for subsequent rounds. The beads were washed three times with PBST (PBS, ThermoFisher Scientific, with 0.02% TritonX-100). Stopped ribosome display solutions were first incubated with antibody-coated beads (without targets) at 4° C. for 30 minutes for pre-clearing of non-specific and off-target binders, the solution were then transferred to target immobilized beads and incubated at 4° C. for 1 hour, the target immobilized beads were then washed 4 times with wash buffer (10 mM HEPES pH 7.4, 150 mM KCl, 5 mM MgCl₂, 0.4 μg/μl BSA (New England Biolabs), 0.1 U/μl SUPERase•In (ThermoFisher Scientific), 0.05% TritonX-100). After washing, beads were resuspended in TRIzol Reagent (ThermoFisher Scientific, 15596026), and RNA was extracted from the beads, 25 μg of linear acrylamide (ThermoFisher Scientific, AM9520) were used as co-precipitant during RNA extraction. Reverse transcription of extracted RNA was performed using Maxima H Minus Reverse Transcriptase (ThermoFisher Scientific) and primer as described in Table 12, row 64. The reverse transcription reaction was purified using SPRIselect Reagent (Beckman Coulter) to obtain purified cDNA. Purified cDNA was amplified by PCR using equal mixtures of Phusion High-Fidelity DNA polymerase and Deep Vent DNA polymerase. PCR cycle number (Table 12) was chosen to avoid over-amplification and typically falls between 10 to 25. This PCR condition ensures efficient full-length product synthesis at each cycle and is required to faithfully amplify nanobody genes without CDR shuffling, a phenomenon¹⁸ that could otherwise cause selection failure. The PCR product was purified by DNA agarose gel extraction. The purified PCR product was used for library generation for high throughput full-length sequencing or as DNA input for ribosome display reaction (coupled in vitro transcription and translation) to perform additional rounds of in vitro selection.

One round of anti-Myc selection was performed on the nanobody library with CDR1 and 2 randomized to enrich for correct-frame sequences. Several factors can in principle contribute to the presence of out-of-frame sequences after anti-Myc selection: (1) Non-specific binding of RNA or protein to magnetic beads; (2) translation through alternative start codons downstream of areas containing out-of-frame errors; and/or (3) inefficient binding of anti-Myc antibody to the expressed Myc peptide that is located between the VHH protein and ribosome. Applicants disfavor (1), because although their input library contained 27.5% full-length sequences, the remaining sequences that contained errors do not interfere with full-length sequences and are reduced to <10% after three rounds of RBD selection (FIG. 6c), suggesting that these erroneous sequences or their encoded peptides do not non-specifically stick to beads at significant levels to impact binder selection.

A control experiment was performed to demonstrate the efficiency of the ribosome display and selection protocol: SR6c3 sequence was linked with 5′ and 3′ sequence elements for ribosome display and serves as control input DNA, 100 ng of control input DNA was displayed by ribosome display in a reaction volume of 10 μl, and bond to 500 μl RBD coated beads, washed and total RNA was extracted from the beads. 7,910 ng total RNA was recovered, among which 989 ng is estimated to be SR6c3 RNA (⅛ of total, calculated by mass ratio of VHH RNA, 649 nt, to E. Coli. ribosomal RNAs, 4,568 nt), representing a coverage rate of 19× in the output.

CDR-directed clustering analysis. Computational analysis for CDR-directed clustering was performed using custom python scripts. Paired end sequences were merged to form full-length VHH sequences. Merged VHH sequences were quality trimmed and translated into VHH protein sequence, which were separated into CDRs and frames (segments) as described in the Amino Acid Profile Construction section. Two VHHs were determined to have similar CDRs via the following steps. First, the ungapped sequence alignment score (match score) was calculated for each CDR of the two VHHs as the sum of BLOSUM62³⁰ amino acid pair scores at each aligned position. (If two CDRs have different lengths, their sequence alignment score was set to −5 by default.) The alignment scores of any two pairs of CDRs were summed to yield three scores, and if at least one of the three was larger than 35 (FIG. 6b), the two VHHs were defined as having similar CDRs. Next, VHHs with similar CDRs were grouped by a two-step process. In the first step, Applicants chose as VHH cluster-forming “seeds” those VHHs that were called as similar to at least 5 other VHHs (all remaining VHHs were not considered for clustering). In the second step, Applicants iteratively selected a seed VHH with at least 5 other similar (>35 match score) seed VHHs, and grouped all of them into one cluster, removing them from the seed VHH pool, and iterated this procedure until no seed VHHs remained. For RBD, there were 83,433 seeds in the first step, and 83,392 were grouped in clusters in the second step. For EGFP, 71,210 of 71,220 seeds were grouped in clusters (Table 15). This heuristic was fast in a standard computing environment with multiprocessing capabilities.

A representative sequence to illustrate each CDR in each cluster was chosen as the most frequent CDR sequence in the cluster (the chose representatives for CDR1,2, and 3 may not necessarily be from the same sequence, and are used only for illustrative purposes for each cluster as in Table 7 and 8; whole VHH sequences were used for gene synthesis and all downstream experiments). A consensus sequence was generated for each CDR, where each position in the CDR was represented by a 6 character string, such that the first and fourth character were the single letter code for the top and the second most abundant amino acid at the position, respectively, and the following two characters (second and third for the most abundant; fifth and sixth for the second most abundant), were their frequency, respectively (ranging from 00 for <34% to 99 for 100%). The consensus sequence for a CDR was recorded as a single “BOO” when the standard deviation of the lengths of all CDRs was greater than 1. CDR scores were calculated by summing a score for each position in the CDR consensus sequence, with scores of 3, 2, 1 for positions where the most abundant amino acid had frequencies greater than 80%, 50%, or less, respectively, and a score of 0 for CDRs with a consensus sequence of a single “BOO” (Table 7 and Table 8). Representative whole nanobody sequence for each cluster was selected as the one with the maximal sum (max-sum) of all CDR similarity score between the nanobody and all other nanobodies in the cluster. This max-sum representative nanobody sequence selection process minimizes the impact of random errors introduced during NGS library preparation and sequencing by imposing a scoring penalty on sequences containing random errors.

Mean distance to diagonal was calculated based on squares of residuals, where the residual=y−x, the distance to diagonal (the half diagonal length of the square of residual)=\sqrt{square of residual/2}. The mean distance to diagonal represents the average distance of data points to the diagonal line in a scatter plots of two amino acid profiles and is a measure of the difference between the two amino acid profiles.

Protein expression and purification. Target proteins used for in vitro selection and ELISA were prepared by transiently transfecting HEK293T cells with plasmids carrying either spike RBD with C-terminal 3×Flag tag and N-terminal signal peptide of spike (RBD-3×Flag), or EGFP with C-terminal 3×Flag tag (EGFP-3×Flag). Cell culture media (for RBD-3×Flag) or lysate of cell pellet (for EGFP-3×Flag) were used for coating magnetic beads or plates. VHHs with C-terminal 6×His tag (VHH-6×His) were purified by expressing in E. coli., followed by purification using HisPur Cobalt Resin (ThermoFisher Scientific, 89964). Briefly, VHH-6×His plasmids were transformed into T7 Express E. Coli. (New England Biolabs, C25661), single colonies were transferred into 10 ml LB media and grown at 37° C. for 2-4 hours (until OD reached 0.5-1), the culture was chilled on ice, then IPTG was added to a final concentration of 10 μM. The culture was then incubated on an orbital shaker at room temperature (RT) for 16 hours. Bacterial cells were pelleted by centrifugation and lysed in B-PER Bacterial Protein Extraction Reagent (ThermoFisher Scientific, 78248) supplemented with rLysozyme (Sigma-Aldrich, 71110), DNase I (New England Biolabs, M0303S), 2.5 mM MgCl₂ and 0.5 mM CaCl₂. Bacterial lysates were cleared by centrifugation and mixed with wash buffer (50 mM Sodium Phosphate pH 7.4, 300 mM Sodium Chloride, 10 mM imidazole) at 1:1 ratio, and then incubated with 40 μl HisPur Cobalt Resin for 2 hours at 4° C. The resins were then washed 4 times with wash buffer. Proteins were eluted by incubating resin in elution buffer (50 mM Sodium Phosphate pH 7.4, 300 mM Sodium Chloride, 150 mM imidazole) at RT for 5 minutes. Purified protein samples were quantified by measuring absorbance at 280 nm on a NanoDrop Spectrophotometer.

ELISA assay for VHH binding to RBD. Maxisorp plates (BioLegend, 423501) were coated with 1 μg/ml anti-Flag antibody (Sigma Aldrich, F1804) in coating buffer (BioLegend, 421701) at 4° C. overnight. Plates were washed once with PBST (PBS, ThermoFisher Scientific, with 0.02% TritonX-100), a 1:1 mixture of HEK293T cell culture media containing secreted RBD-3×Flag and blocking buffer (PBST with 1% nonfat dry milk) was added to the plates and incubated at RT for 1 hour. RBD coated plates were then blocked with blocking buffer at RT for 1 hour. Plates were washed twice with wash buffer and purified VHHs-6×His diluted in blocking buffer were added to the plates and incubated at RT for 1 hour. Plates were washed three times with wash buffer, HRP conjugated anti-His tag secondary antibody (BioLegend, 652503) diluted 1:2000 in blocking buffer was then added to the plates and incubated at RT for 1 hour. Plates were washed three times with wash buffer and TMB substrate (BD, 555214) was added to the plate and incubate at RT for 10 to 20 minutes. Stop buffer (1N Sulfuric Acid) was added to the plates once enough color developed. Quantification of plates was performed by measuring absorbance at 450 nm on a BioTek synergy H1 microplate reader. Data reported were background subtracted. Two levels of background subtraction were performed: (1) subtracting absorbance measured from wells incubated with blocking buffer only (without purified VHHs-6×His) from sample measurements (reflecting background absorbance by plates); and (2) subtracting absorbance from each VHH incubated wells coated only with anti-flag antibody and without RBD (reflecting non-specific binding of each VHH).

Pseudotyped SARS-CoV-2 lentivirus production and lentiviral production for transductions. Lentivirus production was performed as previously described (Gentili et al., 2015). Briefly, HEK293T cells were seeded at 0.8×10⁶ cells per well in a 6 well plate and were transfected the same day with TransIT®-293 Transfection Reagent and a mix of DNA containing 1 μg psPAX, 1.6 μg pTRIP-SFFV-EGFP-NLS and 0.4 μg pCMV-SARS2ΔC-gp41. Medium was changed after overnight transfection. SARS-CoV-2 S pseudotyped lentiviral particles were collected 30-34 hours post medium change and filtered on a 0.45 μm syringe filter. To transduce HEK293T ACE2 the same protocol was followed, with a mix containing 1 μg psPAX, 1.6 μg pTRIP-SFFV-Hygro-2A-TMPRSS2 and 0.4 μg pCMV-VSV-G.

SARS-CoV-2 S pseudotyped lentivirus neutralization assay. The day before the experiment, 5×10³ HEK293T ACE2/TMPRSS2 cells per well were seeded in 96 well plates in 100 μl. On the day of lentivirus harvest, SARS-CoV-2 S pseudotyped lentivirus was incubated with VHHs or VHH elution buffer in 96 well plates for 1 hour at RT (100 μl virus+50 μl of VHH at appropriate dilutions). Medium was then removed from HEK293T ACE2/TMPRSS2 cells and replaced with 150 μl of the VHH+pseudotyped lentivirus solution. Wells in the outermost rows of the 96 well plate were excluded from the assay. After overnight incubation, medium was changed to 100 μl of fresh medium. Cells were harvested 40-44 hours post infection with TrypLE (Thermo Fisher), washed in medium, and fixed in FACS buffer containing 1% PFA (Electron Microscopy Sciences). Percentage GFP was quantified on a Cytoflex LX (Beckman Coulter) and data were analyzed with FlowJo. During development of the pseudotyped lentivirus neutralization assay, Applicants found HEK293T ACE2/TMPRSS2 cells were highly susceptible to pseudovirus infection and produced consistent inhibition measurements, while Vero E6 and Caco-2 cells showed lower susceptibility in Applicants' GFP detection-based assays.

Affinity maturation. Error-prone PCR was used to introduce random mutations across the full length of selected VHH DNA sequences. 0.1 ng of plasmid carrying DNA sequence encoding each selected VHH were used as template in PCR reactions using Taq DNA polymerase with reaction buffer (10 mM Tris-HCl pH 8.3, 50 mM KCl, 7 mM MgCl₂, 0.5 mM MnCl₂, 1 mM dCTP, 1 mM dTTP, 0.2 mM dATP, 0.2 mM dGTP) suitable for causing mutations in PCR products. Mutagenized library for input to CeVICA was made by ligating PCR products of error-prone PCR that carries VHH to DNA fragment containing the remaining elements required for ribosome display. Three rounds of ribosome display and in vitro selection were performed on the mutagenized library (pre-affinity maturation, after error-prone PCR) as described in the In vitro selection section, during which the incubation time of the binding step was kept between 5 seconds to 1 minute to impose a stringent selection condition, additional error-prone PCR was not performed during the selection cycles. The output library (post-affinity maturation) was sequenced along with the pre-affinity maturation library as described in the High throughput full-length sequencing of VHH library section.

Identification and ranking of beneficial mutations. To identify potential beneficial mutations for each selected VHH Applicants built an amino acid profile (a.a. profile) table for each VHH family in the pre- and post-affinity maturation library, and identified amino acids with increased frequency in the post-affinity maturation population compared to their pre-maturation frequency. For each VHH parental sequence, an a.a. profile was built of the percent of each a.a. across all VHH sequences originated from one parental VHH in the pre-affinity maturation library (“pre-a.a. profile”) and in the post-affinity maturation library (“post-a.a. profile”). A percent point change table was generated by subtracting the pre-a.a. profile from the post-a.a. profile, describing the change of frequency of each observed amino acid at each position of the VHH protein following affinity maturation.

Applicants defined a putative beneficial mutation as either (1) the non-parental amino acid with the biggest increase in frequency if its increase is at least 0.5 percentage points; the score is the difference from the parental amino acid frequency; or (2) the non-parental amino acid with the biggest increase after the parental amino acid if the increase is at least 1.5 percentage points; the score is the percent point change of the beneficial mutation. To avoid too many proximal putative beneficial mutations (which may cause structural incompatibility), a putative beneficial mutation was discarded if it (1) is outside the CDRs; (2) is less than 3 positions away from another beneficial mutation (“nearby mutation) and has a smaller beneficial mutation score than the nearby mutation; and (3) co-occurs less than twice with the nearby mutation. From this final list of putative beneficial mutations, different combinations were picked and incorporated into each VHH parental sequence that include one combination of all beneficial mutations in CDRs, one combination of the top-3 ranked (by beneficial mutation score) mutations in frames, and at least one combination of both CDR mutations and frame mutations (Table 9).

Biolayer Interferometry. Biolayer Interferometry assays were performed on the Octet RED384 instrument (sartorius) using anti-GST biosensors (sartorius, 18-5096). Assays were performed in sample buffer (PBS with 0.05% Tween-20, 0.5 mg/ml BSA). VHHs were loaded on anti-GST biosensors in sample buffer containing bacteria lysates of E. Coli. expressing GST tagged VHHs (100-fold dilution), loading level of 1 nm to 1.2 nm were achieved. VHH loaded sensors were dipped in sample buffer containing recombinant RBD (ThermoFisher, RP-87678) for 200 seconds to record association, then dipped in sample buffer for 1200 seconds to record dissociation. VHH loaded sensors dipped in sample buffer containing no RBD were used as reference sample sensors for background subtraction, no signal increase was observed for reference sample sensors which indicates no non-specific binding to loaded VHHs. Non-specific binding of RBD to anti-GST biosensors were tested by dipping anti-GST biosensors not loaded with VHHs in 20 nM RBD, no signal increase was observed during the incubation indicating that RBD does not non-specifically bind to anti-GST biosensors. Data analysis were performed using the Octet Data analysis software 10.0, Savitzky-Golay Filtering was used to remove noise and curves were fitted using 1:1 binding model.

Size exclusion chromatography. Size-exclusion chromatography were performed on an AKTA Pure 25M system with a Superdex increase 75 10/300 GL column (cytiva). 50 μg to 100 ug VHHs were loaded onto the column in running buffer (20 mM HEPES, 150 mM NaCl, PH 7.5), a flow rate of 0.5 ml/min was used and UV280 readings were recorded for 1.25 column volumes. Peak analysis was performed using the UNICORN 7 software (cytiva).

Thermal stability assays. Protein thermal shift assays were performed using the Protein Thermal Shift Dye Kit (ThermoFisher, 4461146) according to the manufacturer's instructions. 4 μg of VHHs were diluted in 1× reaction buffer and measurements were performed on a Bio-Rad CFX384 real-time PCR system using a melt curve protocol (30° C.-98° C., 1° C. increment, hold for 20 seconds then read plates using FRET channel). 98° C. heat denaturation was performed by diluting VHH sample to 1 μM in PBS containing 100 ng/ul BSA, then heating at 98° C. for 10 minutes then holding at 4° C. using a PCR machine. ELISA assay of VHH samples prior to and after complete thermal denaturation were performed as previously described (ELISA assay for VHH binding to RBD).

### Tables

Table 10A-10B. natural nanobody (VHH) sequences selected for calculating natural VHH amino acid profile. Table 10A. Amino acid sequences of 298 unique nanobodies selected from Protein Data Bank to represent natural nanobodies (PDB298, sheet: unique_VHH_PDB) and Table 10B. amino acid sequences of 1,030 unique nanobodies selected from abYsis to represent natural nanobodies (abYsis1030, sheet: unique_VHH_abYsis). The sequences were separated into 4 frames and 3 CDRs. (SEQ ID NOS: 5873-15101)

TABLE 10A

| | | SEQ | | SEQ | | | SEQ | |
|---|---|---|---|---|---|---|---|---|
| | | ID | | ID | | | ID | |
| | | NOS: | SEQ | NOS: | | | NOS: | |
| | SEQ ID NOS: | 6171- | ID NOS: | 6767- | SEQ ID NOS: | SEQ ID NOS: | 7647- | |
| PDB | 5873-6170 | 6468 | 6469-6766 | 7058 | 7059-7356 | 7357-7646 | 7944 | Source |
| ID | Frame1 | CDR1 | Frame2 | CDR2 | Frame3 | CDR3 | Frame4 | Organism |
| 3G9A | DVQLQESGGGSVQA | DTFS | MAWFRQAPGK | RDGT | TTYAGSVKGRFTISRDDAK | DSGTQLGYV | DYWGKGT | Camelus |
| | GGSLRLSCAASG | SYS | ECELVSNIL | | NTVYLQMVNLKSEDTARYY | GAVGLSCLD | QVTVSS | Dromedarius |
| | | | | | CAA | YVM | | (9838) |
| 4I0C | QVQLQESGGGSVQA | LSTTV | MAWFRQAPGK | TGDG | PYYADSVKGRFTISQDNAK | KTGAFSYGS | NHWGQG | Camelus |
| | GGSLRLSCEASG | | EREGVAAIY | F | NRMYLQMNSLEPEDTAMY | LWWMSRAY | TQVTVSS | Dromedarius |
| | | | | | YCAA | | | (9838) |
| 2X6M | GQLVESGGGSVQAG | IDSSS | MGWFRQRPGK | GLGG | TAYADSVKDRFTISRDNAE | KFSPGYCGG | GYWGQG | Camelus |
| | GSLRLSCAASG | YC | EREGVARIN | VK | NTVYLQMNSLKPEDTAIYY | SWSNF | TQVTVSS | Dromedarius |
| | | | | | CAA | | | (9838) |
| 2X89 | QVQLQESGGGSVQA | YTDS | MAWFRQAPGK | SGRDI | TYYADSVKGRFTFSQDNA | DIPLRCRDIV | RYWGQGT | Camelus |
| | GGSLRLSCAASG | RYC | EREWVARIN | | KNTVYLQMDSLEPEDTATY | AKGGDGF | QVTVSS | Dromedarius |
| | | | | | YCAT | | | (9838) |
| 3JBC | QVQLQESGGGSVQT | YTQS | MGWFRQAPGK | RFFGT | AYYADSVKGRFTISQDKAK | GQGCLTTIQA | NAWGQGT | Camelus |
| | GGSLRLSCAASE | SAC | EREGVAGIS | | NTVYLQMNSLKPEDTAIYY | LGGAYGY | QVTVSS | Dromedarius |
| | | | | | CAA | | | (9838) |
| 3JBD | QVQLQESGGGSAQT | FTFS | MAWFRQAPEK | TSGV | AYYADSVLGRFTISQDNAK | TSISCSSGY | MFWGQG | Camelus |
| | GGSLRLSCAASG | HGY | EREWVACVR | S | STLYLQMNNLKPEDTAMYY | | TQVTVSS | Dromedarius |
| | | | | | CAA | | | (9838) |
| 3JBE | QVQLQESGGGSVQA | YQYS | MAWFRQVLGE | TYNG | MRYADTVKGRFTVSQDKD | GRWRFGDIC | NYWGQGT | Camelus |
| | GGSLRLSCAASG | LLC | GREGVAFIT | A | KNTVYLQMNSLKPEDTAIY | HEGSGY | QVTVSS | Dromedarius |
| | | | | | YCAA | | | (9838) |
| 3JBF | QVQLQESGGGSVQA | ITYC | MGWYRQVRGK | TNDG | TDYADSVKGRFTISQNHAT | TWRWSCPLD | QYWGQG | Camelus |
| | GGSLTLSCAASG | | EREGVAFIK | T | KTVYLQMNSLKPEDTAAYY | PEGY | TQVTVSS | Dromedarius |
| | | | | | CAA | | | (9838) |
| 3JBG | QVQLQESGGGSVQA | YIPSA | CMRWFRQAPK | RGGY | TWHADSVKGRFTISGDNA | SNTCPGASA | RSWGQGT | Camelus |
| | GGSLRLSCAASE | N | EREWVASVL | | KTAAYLQMNSLKPEDTAIY | DF | QVTVSS | Dromedarius |
| | | | | | YCAY | | | (9838) |
| 3OGO | QVQLVESGGALVQP | FPVN | MRWYRQAPGK | SAGD | SSYEDSVKGRFTISRDDAR | NVGF | EYWGQGT | Camelus |
| | GGSLRLSCAASG | RYS | EREWVAGMS | R | NTVYLQMNSLKPEDTAVYY | | QVTVSS | Dromedarius |
| | | | | | CNV | | | (9838) |
| 3DWT | QVQLVESGGGSVQA | GSEY | LGWFRQAPGQ | SMGG | TYYADSVKGRFTISRDNAK | VRGYFMRLP | RYWGQGT | Camelus |
| | GGSLRLSCTASG | SYST | EREAVAAIA | L | NTVTLQMNNLKPEDTAIYY | SSHNF | QVTVSS | Dromedarius |
| | | FS | | | CAA | | | (9838) |
| 5C2U | QVQLQESGGGLVQP | DYYA | IGWFRQAPGK | SRDG | TYYPDAVKGRFTISRDNAK | DRRQRWGP | VYWGQGT | Camelus |
| | GGSLRLSCVVSG | | EREGVAAIS | S | NTVYLQMNSLKPEDTAVYY | YYYLSALEY | QVTVSS | Dromedarius |
| | | | | | CAA | | | (9838) |
| 5KWL | QVQLQESGGGSVQP | YAVS | MGWFRQAPGK | SSGV | TTYADSVKGRFTISRDNAK | GFGLSLSRYT | AYWGQGT | Camelus |
| | GGSLTLSCAASG | RYS | ENEGVAAID | G | DTVYLRMNSLKPEDTAIYY | Y | QVTVSS | Dromedarius |
| | | | | | CAS | | | (9838) |
| 5KU2 | QVQLQESGGGSVQT | YAVS | MGWFRQAPGK | SSGV | TTYADSVKGRFTISRDNAK | GFGLSLSRYT | AHWGQGT | Camelus |
| | GGSLTLSCAASG | LYS | ELEGVAGIS | D | DTMYLQMNSPKPEDTAIYR | Y | QVTVSS | Dromedarius |
| | | | | | CAA | | | (9838) |
| 5HGG | QVQLQESGGGLVQA | FTLDS | IGWFRQAPGK | ASGG | TNYADSVKGRFTISRDNAK | DHPGLCTSE | EVWGQGT | Vicugna |
| | GGSLRLSCAASG | YA | EREGVSCIS | S | NTVYLQMNSLKSEDTAVYY | SGRRRYL | QVTVSS | Pacos |
| | | | | | CAA | | | (30538) |
| 5MY6 | QVQLQESGGGSVQA | YIFNS | MGWYRQSPGR | GDGD | TWHKESVKGRFTISQDNVK | CYNLE | TYWGQGT | Camelus |
| | GGSLKLTCAASG | CG | ERELVSRIS | | KTLYLQMNSLKPEDTAVYF | | QVTVSS | Dromedarius |
| | | | | | CAV | | | (9838) |
| 3QSK | QVQLVESGGGLVQA | YHHP | MGWFRQAPGK | SGGG | TLYADSVKGRFTISRDKGK | GGHHLRDHT | GQWGQG | Camelus |
| | GGSLRLSCAASG | YIY | EREGVAAMD | G | NTVYLQMDSLKPEDTATYY | Y | TQVTVSS | Dromedarius |
| | | | | | CAA | | | (9838) |
| 1RI8 | DVQLVESGGGSVQA | YKDR | MGWFRRAPGK | SSGR | TAYADSVKGRFTISRDVAL | GWSSLGSCG | NYWGQGT | Camelus |
| | GGSLRLSCAVSG | NYC | EREGVAVID | | DTAYLQMNSLKPEDTAMY | TNRNRY | QVTVSS | Dromedarius |
| | | | | | YCAA | | | (9838) |
| 1RJC | EVQLQASGGGSVQA | ATSS | MGWFRQAPGK | TGRG | TAYADSVQGRLTISLDNAK | DTSTWYRGY | SYWGQGT | Camelus |
| | GQSLRLSCATSG | SNC | EREGVAVID | N | NTLYLQMNSLKPEDTAMYY | CGTNPNYF | QVTVSS | Dromedarius |
| | | | | | CAA | | | (9838) |
| 1ZMY | QVQLVESGGGSVQA | YTIGP | MGWFRQAPGG | MGGG | TYYADSVKGRFTISRDNAK | DSTIYASYYE | DSWGQGT | Camelus |
| | GGSLRLSCTASG | YC | EREAVAAIN | I | NTVTLQMNSLKPEDTAMY | CGHGLSTGG | QVTVSS | Dromedarius |
| | | | | | YCAA | YGY | | (9838) |
| 1MVF | QVQLVESGGGSVQA | FTYS | MGWFRQAPGK | IDNGN | TIYADSVQGRFTISQDNAK | SSRWMDYSA | NSWGQGT | Camelus |
| | GGSLRLSCAASG | RKY | EREGVAAIF | | NTVYLQMNSLKPEDTAMY | LTAKAY | QVTVSS | Dromedarius |
| | | | | | YCAA | | | (9838) |
| 1KXT | QVQLVASGGGSVQA | YTFS | MGWYRQAPGK | SDGS | ANYADSVKGRFTISRDNAA | GPGSGKLVV | NYWGQGT | Camelus |
| | GGSLRLSCAASG | SYP | ECELSARIF | | NTAYLQMDSLKPEDTAVYY | AGRTCYGP | QVTVSS | Dromedarius |
| | | | | | CAA | | | (9838) |
| 1KXQ | QVQLVESGGGSVQA | YTDT | VGWFRQAPGK | RRTG | TYSADSVKGRFTLSQDNN | GNSVRLASW | FYWGQGT | Camelus |
| | GGSLSLSCAAST | | EREGVAAIY | Y | KNTVYLQMNSLKPEDTGIY | EGY | QVTVSS | Dromedarius |
| | | | | | YCAT | | | (9838) |
| 1KXV | QVQLVESGGGTVPA | NTLC | MSWYRRAPGK | NDGT | TTYVDSVAGRFTISQGNAK | SLRYGLPGC | IPWGQGT | Camelus |
| | GGSLRLSCAASG | TYD | GRDFVSGID | | NTAYLQMDSLKPDDTAMY | PI | QVTVSS | Dromedarius |
| | | | | | YCKP | | | (9838) |
| 1XFP | DVQLQASGGGSVQA | YTIGP | MGWFRQAPGK | SGGG | TYYADSVKGRFTISQDNAK | DSTIYASYYE | DSWGQGT | Camelus |
| | GGSLRLSCAASG | YC | EREGVAAIN | S | NTVYLLMNSLEPEDTAIYY | CGHGLSTGG | QVTVSS | Dromedarius |
| | | | | | CAA | YGY | | (9838) |
| 3EBA | QVQLVESGGGSVQA | YTYI | SGWFRQAPGK | SSDG | TYYADSVKGRFTISQDNAK | TEVAGWPLDI | DYWGQGT | Camelus |
| | GGSLRLSCSASG | | GLEWVAAIR | T | NTVYLQMNSLKPEDTAMY | GIY | QVTVSS | Dromedarius |
| | | | | | YCAA | | | (9838) |
| 1G6V | QVQLVESGGGSVQA | YTVS | MGWFRQAPGK | GGS | TYYGDSVKGRFTISQDNAK | STVASTGWC | HYRGQGT | Camelus |
| | GGSLRLSCAASG | TYC | EREGVATIL | | NTVYLQMNSLKPEDTAIYY | SRLRPYDY | QVTVSS | Dromedarius |
| | | | | | CAG | | | (9838) |
| 2P46 | QVQMVESGGGLVQA | YAYT | MGWFRQAPGK | SGGG | TLYADSVKGRMTISRDKGK | GGYELRDRT | GQWGQG | Camelus |
| | GGSLRLSCAASG | YIY | EREGVAAMD | G | NTVYLQMDSLKPEDTATYY | Y | TQVTVSS | Dromedarius |
| | | | | | CAA | | | (9838) |
| 6KSN | QVQLEESGGGSVQA | YMYS | MGWFRQAPGK | RGDH | TYYTDSVKGRFTISQDSAK | DFAHSFLLSV | SYWGQGT | Camelus |
| | GGSLRLSCAASG | TYST | EREGVAFIK | S | NTVSLQMNNLKPEDTAIYY | HSGAGQY | QVTVSS | Dromedarius |
| | | YC | | | CAA | | | (9838) |
| 5U65 | DVQLQESGGGSVQA | LTISN | MRWFRQAPGK | SAGT | TYYADSVKGRFTMSRDNA | STRVWGGYC | NDWGQG | Camelus |
| | GGSLRLSCVVSG | YC | GREGVASIN | | KNTVYLDMNSLKPEDTAIY | GGLDDATN | TQVTVSS | Dromedarius |
| | | | | | YCAS | | | (9838) |
| 5U64 | DVQLQESGGGSVQA | VTST | IGWFRQAPGK | FRGD | TYITDSVKGRFTISRDEDSD | DVNRGGFCY | SYWGQGT | Camelus |
| | GGSLRLSCVASG | RPC | EREGVAVVN | S | TVYLQMNSLKPEDTATYYC | IEDWYF | QVTVSS | Dromedarius |
| | | | | | AA | | | (9838) |
| 1ZVY | DVQLVESGGGSVQA | STDSI | MTWFRQAPGK | THTG | TYYTDSVKGRFTISQDKAK | TRKYVPVRF | DYWGQGT | Camelus |
| | GGSLRLSCAASG | EY | AREGVAALY | N | NMAYLRMDSVKSEDTAIYT | ALDQSSY | QVTVSS | Dromedarius |
| | | | | | CGA | | | (9838) |
| 6JB5 | DVQLVESGGGSVQA | STDSI | MTWFRQAPGK | THTG | TYYTDSVKGRFTISQDKAK | TRKAVPVRF | DYWGQGT | Camelus |
| | GGSLRLSCAASG | EY | AREGVAALY | N | NMAYLRMDSVKSEDTAIYT | ALDQSSY | QVTVSS | Dromedarius |
| | | | | | CGA | | | (9838) |
| 6DBA | QVKLEESGGGSVQA | HTYS | MGWFRQVPGK | VGGS | TWYADSVRDRFTISQDNAK | HRFCNTWSL | NVWGQGT | Camelus |
| | GGSLRLSCAASG | TYC | EREGVARIN | S | NTVYLQMNSLKLEDTAIYY | GTL | QVTVSS | Dromedarius |
| | | | | | CTL | | | (9838) |
| 5FOJ | QVQLQESGGGSVQV | DTFS | AAWFRQAPGK | SKRH | TSYADSVKGRFTISKDNAD | ADAIGLAEYW | KYWGQGT | Camelus |
| | GGSLRVACAASG | GYL | GREGVAAIN | T | NIMYLEMNSLKPEDTAIYYC | STPTLSAARY | QVTVSS | Dromedarius |
| | | | | | AA | | | (9838) |
| 5FUC | DVQFVESGGGSVHA | YIYST | MGWFRQAPGK | TNSG | TYYADSVKGRFTISQDNAK | RPSIRCASFS | KDWGQGT | Camelus |
| | GGSLRLNCATSG | YC | EREGVAHIY | R | NTVYLQMNSLKPEDTAIYY | ATEY | QVTVSS | Dromedarius |
| | | | | | CAA | | | (9838) |
| 5E7F | QVQLVESGGGSVQA | FTFD | MGWYHQAPGN | SDGS | TYYADSVKGRFTISRDNAK | ATTTVASPPV | GYWGQG | Camelus |
| | GGSLRLSCTASG | DSD | ECELVSAIF | | NTVYLQMNSLKPEDTAMY | RHVCN | TQVTVSS | Dromedarius |
| | | | | | YCAA | | | (9838) |
| 6XXP | QVQLQESGGGSVEA | WPYS | MNWFRQAPGK | STMS | IIFAESKAGQFTISQDNAKN | RRDYSLSSS | DYWGQGT | Camelus |
| | GGSLRLSCARSG | TYS | EREAVAGIS | G | TVYLQMNNLKPEDTAIYYC | SDDF | QVTVSS | Dromedarius |
| | | | | | AA | | | (9838) |
| 6XXN | QVQLQESGGGSVQA | YTDS | MSWFRQAPGK | TGRG | TSYADSVKGRFTISQDNAK | AACHFCDSL | ILWGQGT | Camelus |
| | GGSLRLSCTAPG | NYY | EREWVAGVN | S | NTMFLQMNSLKPEDTAIYY | PKTQDEY | QVTVSS | Dromedarius |
| | | | | | CAV | | | (9838) |
| 4KML | AVQLQESGGGLVQP | RTFS | MGWFRQAPGK | SSGD | SDYTDSVKGRFTISRDNAK | GLGIYIIRARG | DHWGQG | Lama |
| | GGSLRLSCAASG | SYN | GREFVASIT | K | NTMYLQMNNLKPEDTATY | GY | TQVTVSS | Glama |
| | | | | | YCAR | | | (9844) |
| 1YC7 | DVQLVESGGGSVQA | STYS | TGWYRQAPGK | SPGT | IYYQDSVKGRFTISRDNAK | QCGVRSIR | EYWGQGT | Camelus |
| | GGSLRLSCAVSG | PCT | EREWVSSIS | | NTVYLQMNSLQREDTGMY | | QVTVSS | Dromedarius |
| | | | | | YCQI | | | (9838) |
| 6U14 | QVKLEESGGGSVQA | HTYS | MGWFRQVPGK | VGGS | TWYADSVRDRFTISQDNAK | HRFANTWSL | NVWGQGT | Camelus |
| | GGSLRLSCAASG | TYA | EREGVARIN | S | NTVYLQMNSLKLEDTAIYY | GTL | QVTVSS | Dromedarius |
| | | | | | CTL | | | (9838) |
| 2P4A | QVQLVESGGGLVQA | YPWT | MGWFRQAPGK | SGGG | TLYADSVKGRFTISRDKGK | GGDALVATR | GRWGQG | Camelus |
| | GGSLRLSCAASG | YIY | EREGVAAMD | G | NTVYLQMDSLKPEDTATYY | Y | TQVTVSS | Dromedarius |
| | | | | | CAA | | | (9838) |
| 1ZVH | DVQLVESGGGSVQA | YIASI | LGWFRQAPGK | PAGG | PYYADSVKGRFTVSLDNAE | ARQGWYIPL | NYWGQGT | Camelus |
| | GGSLRLSCAASG | NY | EREGVAAVS | T | NTVYLQMNSLKPEDTALYY | NSYGY | QVTVSS | Dromedarius |
| | | | | | CAA | | | (9838) |
| 1ZV5 | DVQLVESGGGSVQA | VTYK | IGWFRQAPGK | SDGGI | TYYADSVKGRFTISQDNAK | GYRNYGQCA | RYWGQGT | Camelus |
| | GESLRLSCAASG | NYC | DREGVVFIN | | NTVYLQMNSLKPEDTASYY | T | QVTVSS | Dromedarius |
| | | | | | CAA | | | (9838) |
| 4OCN | QVQLQESGGGLVPA | RTFS | MAWFRQAPGK | WSGG | TYYADSVKGRFTISRDNAR | GTYYGTLSY | DFWGRGT | Lama |
| | GGSLRLSCVDSG | STV | EREFVATIR | N | NTVYLQMNSLKPEDTAVYY | KY | QVTVSS | Glama |
| | | | | | CAG | | | (9844) |
| 4LAJ | EVQLVESGGGLVQP | FTLDY | IGWFRQAPGK | DSDG | TYYADSVKGRFTISRDNAK | DCTVDPSLLY | DYYGKGT | Lama |
| | GGSLRLSCAASG | YS | EREGVSCIS | R | NTVYLQMNSLKPEDTAVYY | VM | QVTVSS | Glama |
| | | | | | CAT | | | (9844) |
| 6H7L | QVQLQESGGGLVQA | SIFAL | MGWYRQAPGK | SGGT | TNYANSVKGRFTISRDNAA | KDFGAIIYDY | DYWGQGT | Lama |
| | GGSLRLSCAASG | NI | QRELVAAIH | | NTVYLQMNSLKPEDTAVYY | | QVTVSS | Glama |
| | | | | | CNV | | | (9844) |
| 4KRM | QVKLEESGGGSVQT | RTSR | MGWFRQAPGK | WRGD | TGYADSVKGRFTISRDNAK | AAGSAWYGT | DYWGQGT | Lama |
| | GGSLRLTCAASG | SYG | EREFVSGIS | S | NTVDLQMNSLKPEDTAIYY | LYEY | QVTVSS | Glama |
| | | | | | CAA | | | (9844) |
| 4KRO | QVQLQESGGGLVQP | RTFS | MGWFRQAPGK | WSGG | TYYTDSVKGRFTISRDNAK | TYLSSDYSRY | DYWGQGT | Lama |
| | GGSLRLSCAASG | SYA | QREFVAAIR | Y | TTVYLQMNSLKPEDTAVYY | ALPQRPLDY | QVTVSS | Glama |
| | | | | | CAA | | | (9844) |
| 4KRP | EVQLVESGGGLVQA | RTFS | MGWFRQAPGK | WSSG | TYYADSVKGRFTISRDNAK | GYQINSGNY | DYWGQGT | Lama |
| | GGSLRLSCAASG | SYA | EREFVVAIN | S | NTMYLQMNSLKPEDTAVY | NFKDYEY | QVTVSS | Glama |
| | | | | | YCAA | | | (9844) |
| 4KSD | QVQLQESGGGLVQA | RTFN | MGWFRQAPGK | WSGE | TYYADSVKGRFTISRDNAK | RLTLGQF | DYWGQGT | Lama |
| | GGSLRLSCAASG | SAV | ERQFVATID | Y | NTVYLQMTSLKPEDTALYY | | QVTVSS | Glama |
| | | | | | CAA | | | (9844) |
| 4NBY | QVQLVESGGGLAQA | RTFS | MAWFRQPPGK | STGR | TYYADSVKGRFTISRDNAK | APYGANWYR | AYWGQGT | Lama |
| | GGSLRLSCAASG | MDP | EREFVAAGS | T | NTVYLQMNSLKPEDTAVYY | DEY | QVTVSS | Glama |
| | | | | | CAA | | | (9844) |
| 4NC0 | QVKLEESGGGLVQA | RTFS | VAWFRQAPGA | STGTS | TYYADSVKGRFTISRDNAK | NSQRTRLQD | DYWGQGT | Lama |
| | GGSLRLSCAASE | RYP | EREFVAVIS | | VTVYLQMNNLKREDTAVYF | PNEY | QVTVSS | Glama |
| | | | | | CAV | | | (9844) |
| 4N1H | QVQLQESGGGLVQA | RTFS | MGWFRQAPGK | RSGG | TKYADSVKGRFAISRDNDK | TTYASLSDTY | DDWGQG | Lama |
| | GASLKLSCAASG | SYA | EREFVAAIS | D | NTVWLRMNSLKPEDTAVY | IGEHIY | TQVTVSS | Glama |
| | | | | | YCAA | | | (9844) |
| 4MQT | QVQLQESGGGLVQA | FDFD | IGWFRQAPGQ | PSDG | TIYADSAKGRFTISSDNAEN | WTLFHSD | EYWGQGT | Lama |
| | GDSLRLSCAASG | NFDD | EREGVSCID | S | TVYLQMNSLKPEDTAVYVC | | QVTVSS | Glama |
| | | YA | | | SA | | | (9844) |
| 3R0M | AVQLQESGGGLVQA | RISSS | MGWFRQAPGK | WSGG | TDYADSVKGRFAISKDNAK | KWRPLRYSD | YDWGQGT | Lama |
| | GGSLRLSCTASG | YD | EREFVAAIS | T | NAVYLQMNSLKPEDTAVYY | YPSNSDY | QVTVSS | Glama |
| | | | | | CAA | | | (9844) |
| 4I13 | QVQLQESGGGLVQA | RLTV | IGWFRQAPGK | WGGG | TVYGESVEGRFTISRDIAKN | SRISYRVWN | TLWGRGT | Lama |
| | GASLRLSCAASE | DYA | EREFVAAIS | L | TMNLQMNVLRPEDTANYY | TIPYNKL | QVTVSS | Glama |
| | | | | | CAA | | | (9844) |
| 4I1N | QVQLQESGGGLVQA | RLTV | IGWFRQAPGK | WGGG | TVYGESVEGRFTISRDIAKN | SRISYAVWNT | TLWGRGT | Lama |
| | GASLRLSCAASE | DYA | EREFVAAIS | L | TMNLQMNVLRPEDTANYY | IPYNKL | QVTVSS | Glama |
| | | | | | CAA | | | (9844) |
| 4IDL | KVQLQQSGGGAVQT | NTASI | MGWYRRAPGK | TTGT | ADYGDFVKGRFTISRDNAN | DGRRFDGAR | ESWGQGT | Lama |
| | GGSLKLTCLASG | RA | QREWVASLT | | NAATLQMDSLKPEDTAVYY | WREY | QVTISS | Glama |
| | | | | | CNA | | | (9844) |
| 4IOS | QVQLVESGGGLVQA | RTFS | IGWFRQAPGK | WSTG | TYYGRSMKGRCAASRDNA | TLDWGKTLS | DYWGQGT | Lama |
| | GDSLRLSCAVSG | SNV | EREFVAAIS | S | KNTVALQLNSLKPEDTAVY | DEY | QVTVSS | Glama |
| | | | | | YCAA | | | (9844) |
| 4ORZ | EVOLVESGGGLVQA | FTFG | MAWLRQAPGK | ISGY | TDYADSVKDRFTISRDNAK | RVGSEY | DLWGQGT | Lama |
| | GGSLRLFCAASG | TSN | RREWVALIT | | NTVSLQMNSLKPEDTAIYF | | QVTVSS | Glama |
| | | | | | CAR | | | (9844) |
| 4PIR | DVQLVESGGGLVQP | SLFSI | MDWYRQAPGK | RDAA | TNYADSVKGRFTISRDSAK | DARTITGRA | DYWGQGT | Lama |
| | GGSLRLSCAYSG | LR | ERELVAGIT | GYAD | NTVYLQMNSLKPEDTAVYY | | QVTVSS | Glama |
| | | | | S | CNA | | | (9844) |
| 4POU | QVQLVESGGGLVQA | GYPH | MGWFRQAPGK | SGGG | TLYADSVKGRFTISRDKGK | GGYQLRDRT | GHWGQG | Lama |
| | GGGSLRLSCAAS | PYLH | EREGVAAMD | G | NTVYLQMDSLKPEDTATYY | Y | TQVTVSS | Glama |
| | | | | | CAA | | | (9844) |
| 4POY | QVQLVESGGGLVQA | YPHP | MGWFRQAPGK | SGGG | TLYADSVKGRFTISRDKGK | GGYQLRDRT | GHWGQG | Lama |
| | GGSLRLSCAASG | YLH | EREGVAAMD | G | NTVYLQMDSLKPEDTATYY | Y | TQVTVSS | Glama |
| | | | | | CAA | | | (9844) |
| 4PPT | QVQLVESGGGLVQA | YPHP | MGWFRQAPGK | SGGG | TLYADSVKGRFTISRDKGK | GGYELRDRT | GHWGQG | Lama |
| | GGSLRLSCAASG | YLH | EREGVAAMD | G | NTVYLQMDSLKPEDTATYY | Y | TQVTVSS | Glama |
| | | | | | CAA | | | (9844) |
| 4W81 | EVQLVESGGGLVQA | RTFS | MGWFRQAPGK | AAPG | AFYADSVRGRFSISADSAK | DLKMQVAAY | DYWGQGT | Lama |
| | GDSLRLSATASG | RAV | EREFVAAIS | TAYY | NTVYLQMNSLKPEDTAVYY | MNQRSV | QVTVSS | Glama |
| | | | | | VAA | | | (9844) |
| 3EZJ | QVQLQESGGGLVQA | SIFSI | MDWDRQAPGK | SGGS | TNYADSVKGRFTISRDNAK | NVKTWAGMT | DYWGQGT | Lama |
| | GGSLRLSCAASG | NS | QRELVATIT | | NTVYLQMNSLKPEDTAVYY | R | QVTVSS | Glama |
| | | | | | CNA | | | (9844) |
| 2X1O | QVQLQESGGGLVQA | RNLR | MGWFRQAPGK | WSSD | IYYADSVKGRFIISRDNAKN | GAGWAGTM | NYWGQGT | Lama |
| | GGSLRLSCAAAG | MYR | EREFVGTMV | T | TVYLQMNSLKPEDTAVYYC | TDY | QVTVSS | Glama |
| | | | | | AA | | | (9844) |
| 2X1P | QVQLQESGGGLVQA | RTFT | MGWFRQAPGK | RSGTL | TRYADSAKGRFTISVDNAK | DLHRPYGPG | DSWGQGT | Lama |
| | GGSLRLSCAASG | SFA | EREFVASIS | | NTVSLQMDNLNPDDTAVY | TQRSDEY | QVTVSS | Glama |
| | | | | | YCAA | | | (9844) |
| 2X1Q | QVQLQESGGGLVQP | RTFS | MGWFRQAPGK | QSGA | TAYADSVKGRFTFSRDNAK | SSRVFYTEVL | DYWGQGT | Lama |
| | GGSLRLSCAASG | NYR | EREFVATIS | A | NLLYLEMLSLEPEDTAVYY | QTTTGY | QVTVSS | Glama |
| | | | | | CAA | | | (9844) |
| 2BSE | QVQLQESGGGLVQA | RTGS | MGWFRQLAGK | FDYD | TYYADSVKGRFTVSRDSG | RSGGFSSNR | DGWGQG | Lama |
| | GGSLRLSCTASR | NWC | EPELVVALN | M | KNTVYLQMNSLKPEDTAIY | ELY | TQVTVSS | Glama |
| | | | | | YCAA | | | (9844) |
| 2XA3 | AVQLQESGGGLVQA | RTSS | MGWFRQAPGK | WSGG | TNYVDSVKGRFDISKDNAK | KWRPLRYSD | NYWGQGT | Lama |
| | GGSLRLSCTVSA | SHD | EREFVAAIS | T | NAVYLQMNSLKPEDTAVYY | NPSNSDY | QVTVSS | Glama |
| | | | | | CAA | | | (9844) |
| 3STB | QVQLQESGGGLVQA | RTLS | MGWFRQAPGK | RSGS | TFYADAVKGRFTISRDNAK | DRFSPVVPG | DSWGQGT | Lama |
| | GGSLRLSCAASG | SYA | EREFVAAIN | | NTVYLQMNSLKPEDTAAYY | PIPVNTV | QVTVSS | Glama |
| | | | | | CAA | | | (9844) |
| 4Y8D | QVQLQESGGGSVQA | YTSR | MGWFRQAPGA | RQSN | SYYSDSVRGRFTISQDNAK | TTDCAAFVE | ITAGQGTQ | Lama |
| | GGSLRLSCGASE | | EREGVACIH | L | TTAFLLMSSLKPEDTAIYYC | RATA | VTVSS | Glama |
| | | | | | AT | | | (9844) |
| 3K3Q | AVQLVDSGGGTLQA | LAFD | MGWFRQAPGK | PRTD | TYYAESLEGRFSVSRDAAA | DEDVTPRVM | DHWGQG | Lama |
| | GKSLRLSCAISG | GGAM | DREFVAAIS | E | TMVFLQADNVRLDDTASYY | GVIPHA | TLVTVSS | Glama |
| | | GSEH | | | CAA | | | (9844) |
| | | RLTA | | | | | | |
| | | GA | | | | | | |
| 3K81 | EVQPQESGGGLAQA | ITFAS | WGWYRRAPGK | NEGR | TNYVDSVKGRFTVSRDNA | NLQTGTLSG | LYWGQGT | Lama |
| | GGSLRLSCVVSG | EA | QRELIAAIN | | KNVMYLQMNSLKPEDTAV | AR | QVTVSS | Glama |
| | | | | | YYCNA | | | (9844) |
| 4DKA | QVQLQESGGGLVQA | RTSS | MGWFRQAPGK | RNGA | TYYTDSVKGRFTISRDNAK | DRFPTMEVV | DYWGQGT | Lama |
| | GGSLRLSCAASG | LYS | EREFVAAIS | N | NTVELQMNSLKPEDTAVYY | TIMTNEY | QVTVSS | Glama |
| | | | | | CAA | | | (9844) |
| 5M2M | QVQLQESGGGLVQP | RTFS | IGWFRQAPGK | WSSG | TYYADSVKGRFAISRDIAKN | RDGIPTSRSV | NYWGQGT | Lama |
| | GGSLRLSCAASG | DHSG | EREFVARIY | N | TVDLTMNNLEPEDTAVYYC | ESY | QVTVSS | Glama |
| | | YTYT | | | AA | | | (9844) |
| 5M2I | VQLVESGGGLVQAG | RSLS | MGWFRQAPGK | WRGY | IYYKDSVKGRFTISRDDAK | SILPLSDDPG | TYWGQGT | Lama |
| | GSLSLSCSASG | NYY | ERELLGNIS | N | NTIYLQMNRLKPEDTAVYY | WN | QVTVS | Glama |
| | | | | | CAA | | | (9844) |
| 4WGW | QVQLQESGGGLVQA | SIFSI | ANWYRQPPGM | RDGN | ANYADSVKGRFTISRDRAR | AIRTTVRTSA | EYWGQGT | Lama |
| | GGSLRLSCAASR | DT | QRELVATIT | | NTVYLQMNSLKPEDTGVY | Q | QVTVSS | Glama |
| | | | | | YCNA | | | (9844) |
| 6B73 | QVQLVESGGGLVRP | RTSY | MGWFRRAPGK | WSGI | PTYADSVADRFTTSRDVAN | RAPVGQSSS | DYWGQGT | Lama |
| | GGSLRLSCVDSE | P | EREFVASIT | D | NTLYLQMNSLKHEDTAVYY | PYDY | QVTVSS | Glama |
| | | | | | CAA | | | (9844) |
| 5JQH | QVQLQESGGGLVQA | SIFSL | MGWYRQAPGK | SGGS | TKYADSVKGRFTISRDNAK | KVAGTFSIY | DYWGQGT | Lama |
| | GGSLRLSCAASG | ND | LRELVAAIT | | NTVYLQMNSLKAEDTAVYY | | QVTVSS | Glama |
| | | | | | CNA | | | (9844) |
| 5F1O | DVQLQESGGGLVQA | SRFD | MGWFRQAPGK | WSSG | TRYLDTVKGRFTISRDNAK | RYQPRYYDS | DNWGQG | Lama |
| | GHSLRLSCVGSG | NYA | EREFVAAIS | T | STVYLQMNSLKPEDTAVYY | GDMDGYEY | TQVTVSS | Glama |
| | | | | | CAA | | | (9844) |
| 5F9D | QVQLQESGGGLVQP | SIFSG | MGWYRQAPGK | PQGV | PNYADSVKGRFTISRDNAK | LPNY | RSWGQGT | Lama |
| | GGSLRLSCAASG | NV | LREWVAAIT | | NMLYLQMSSLKPEDTALYY | | QVTVSS | Glama |
| | | | | | CNR | | | (9844) |
| 5F7L | QVQLQESGGGLVQP | SIYSLI | MGWYRQAPGK | SGST | TYYADSVKGRFTISRDNAK | YSDRLTDCS | DYWGQGT | Lama |
| | GGSLRLSCAASG | A | EHELVATIS | | NTLYLQMNSLKPEDTAMYY | NCEA | QVTVSH | Glama |
| | | | | | CAA | | | (9844) |
| 5H8D | QVQLQESGGGLVQP | FTFS | MSWYRQAPGK | SGGG | IYYADSVKGRFTISREDAKN | G | GFWGQGT | Lama |
| | GGSLKLSCAASG | RYA | ERESVARIS | T | TVYLQMNSLKPEDTAVYYC | | QVTVSS | Glama |
| | | | | | YV | | | (9844) |
| 5HM1 | DVQLQESGGGLVQP | NIVSI | AGWFRQAPGK | TGGA | TNYADSVKGRFTISRDNAK | PMIYYGGRY | DYWGQGT | Lama |
| | GGSLRLSCAASG | DA | QREPVATIL | | NTVYLQMNSLKPEDTAVYY | S | QVTVS | Glama |
| | | | | | CYA | | | (9844) |
| 5LWF | QVQLQESGGGLVQA | RTFS | MGWFRKAPGK | PGTE | TYYADSVKGRFAFSRDNA | TPYYRGSYY | TYWGQGT | Lama |
| | GGSLRLSCAASG | NDH | EREFVAAIT | K | KNTVYLQMNSLKPEDTAVY | AASTY | QVTVSS | Glama |
| | | | | | YCVA | | | (9844) |
| 5M30 | QVQLVESGGGLVQA | SISGI | MAWYRQAPGK | SGGT | TNYADSVKGRFTISKDNAE | FFRRDYVGY | DYWGQGT | Vicugna |
| | GGTLKLSCAASG | VV | QRELVASIT | | NTLYLRMNSLKPEDTAVYY | | QVTVSS | Pacos |
| | | | | | CKA | | | (30538) |
| 5MWN | QVQLVESGGGLVQP | IMLGY | MAWYRQAPGK | SGGS | ANYADAVKGRFTISRDNAR | RIWRGTVYD | NISGPGTQ | Lama |
| | GGSLRLSCAASG | FT | QRELVATEI | | STVYLQMNSLKPEDTAVYY | | VTVSS | Glama |
| | | | | | CDA | | | (9844) |
| 5NML | QVQLVESGGGLVQA | SAVS | MGWYRQAPGK | NSGR | TNYVDSVKGRFTISKDNAK | LGATTMSN | AVWGQGT | Lama |
| | GGSLRLSCVVSG | DYA | QRELVAAIY | | KTVYLQMNCLKPEDTADYF | | QVTVSS | Glama |
| | | | | | CNL | | | (9844) |
| 5IP4 | QVQLQESGGGLVQA | NIIGG | WAWYRQAPGQ | RYN | ANYADSVKGRFTISRDNAK | EN | YYWGQGT | Lama |
| | GGSLRLSCTASV | NH | QRDLVASLS | | NAAYLQMNSLKPEDTAIYF | | QVTVSS | Glama |
| | | | | | CAL | | | (9844) |
| 4GFT | QVQLQESGGGTVQP | ERAF | MGWFRQAPGQ | GSGR | QYYADSVKGRFTISRDNAM | RVVPVFSDS | VYWGQGT | Lama |
| | GGSLKLSCSAAP | SNYA | EREFVAGIT | S | NAVYLQMNSVKAEDTAVY | TKGY | QVTVSS | Glama |
| | | | | | YCAA | | | (9844) |
| 4GRW | EVQLVESGGGLVQP | FTLD | IAWFRQAPGK | SGDG | AYYADSVKGRFTISSDNAK | VRTGWGLNA | DYWGKGT | Lama |
| | GGSLRLSCAASG | DYA | EREGVSGID | S | NTVYLQMNSLRPEDTAVYY | PDYAM | LVTVSS | Glama |
| | | | | | CAR | | | (9844) |
| 4GRW | EVQLVESGGGLVQP | FTLDY | IGWFRQAPGK | SSGQ | TYYADSVKGRFTISRDNAE | DPECYRVRG | DYWGQGT | Lama |
| | GGSLRLSCAASG | LA | EREGVSCVS | Y | STVYLQMNSLKPEDTAVYY | YYNGEY | QVTVSS | Glama |
| | | | | | CAT | | | (9844) |
| 4GRW | EVQLVESGGGLVQA | RTFS | VGWFRQAPGK | WSGG | PYYADSVKDRFTISRDNAK | TSLFPTSRGS | DTWGQGT | Lama |
| | GGSLRLSCAASG | WSA | EREFVAAIR | S | NTVYLQMNSLRPEDTAVYL | HY | QVTVSS | Glama |
| | | | | | CGE | | | (9844) |
| 4HEM | QVQLVESGGGLVQA | STFS | MGWFRQAPGP | QTGS | TYYRNSVKGRFTISRDNAK | GDNYYYTRT | DYWGQGT | Lama |
| | GGSLRLSCAASE | NYA | EREFVATIS | H | NTVYLQMNNMKPEDTAVY | YEY | QVTVSS | Glama |
| | | | | | YCAA | | | (9844) |
| 4HEP | DVQLVESGGGLVQP | FSFD | IGWFRQAPGK | MSDG | TYVADSVTGRFTISSDNAK | GRFVTFGSA | DYWGKGT | Lama |
| | GGSLRLSCEASG | DYA | EREGVSYIS | R | NTVYLQMNSLKLEDTAVYY | WSFVGGGPY | LVTVSS | Glama |
| | | | | | CAA | GI | | (9844) |
| 4FHB | QVQLQESGGGLVQA | RTFS | MGWFRQAPGK | WSGS | TYYADSAKGRFTISRDNAK | PGRPHGSSW | DYWGQGT | Lama |
| | GGSLRLSCEASG | SYA | ERDFVAVIS | N | NTVYLQMNSLKPEDTAIYY | SLNKKGQGY | QVTVSS | Glama |
| | | | | | CAA | | | (9844) |
| 4BFB | QVQLQESGGGLVQP | FTFSS | MTWVRQAPGK | SDGSI | TTYADSVKGRFTISRDNAR | A | GRRGPGT | Lama |
| | GGSLRLSCAASG | AI | GREWVSTIG | | NTLYLQMNSLKPEDTAVYY | | QVTVSS | Glama |
| | | | | | CTS | | | (9844) |
| 3QXW | QVQLVESGGGLVQA | RSSR | MAWFRQAPGK | GDGR | TTYGDSVKGRFTISRDNAE | DDNYVTASW | DYWGQGT | Lama |
| | GGSLRLSCAASR | SWA | EREFVAKIS | L | YLVYLQMDSLKPEDTAVYY | RSGP | QVTVSS | Glama |
| | | | | | CAA | | | (9844) |
| 4EIZ | QVQLQESGGGLVQA | RTFS | MGWFRQTPGK | WGGS | TLYADSVKGRFTMSRDNA | DGSQYRSTY | GSWGQG | Lama |
| | GGSLRLSCTASG | SYA | EREFVAAIT | T | KNTVYLQMNSLKPEDTAVY | SFRDKPDY | TQVTVSS | Glama |
| | | | | | YCAA | | | (9844) |
| 4EIG | QVQLQESGGGLVQA | IIFSV | MTWYRQAPGK | TNNN | TMTVDSVKGRFTISRDNVQ | NRGLAGP | AYWGQGT | Lama |
| | GGSLRLSCKASG | YK | ERELVALIT | | NTVYLEMNNLKPEDTAVYY | | QVTVSS | Glama |
| | | | | | CNA | | | (9844) |
| 5DA0 | QVQLQESGGGLVQA | RTFS | MGWFRQAPGK | RSGG | SYNADSVKGRFTISRDNAK | GDTAITSWY | DYWGQGT | Lama |
| | GGSLRLSCAASG | SDV | EREFVAAVT | K | NTVSLQMNSLKPEDTAVYY | GY | QVTVS | Glama |
| | | | | | CAA | | | (9844) |
| 5DFZ | QVQLQESGGGLVQP | PFSP | MGWYRQAPGK | IDGR | TNYQDSVKGRFTISRDYVK | ETRGF | MHWGQG | Lama |
| | GGSLRLSCAPSG | NS | QRELVAVMT | | NTAYLQMNNLKPDDTAVYI | | TQVTVSS | Glama |
| | | | | | CNA | | | (9844) |
| 4B50 | EVQLVESGGGLVQP | SISSV | MSWYRQAPGK | DRGR | TNYKVSVKGRFTISRDNSK | ESRTSWSSP | DVWGRGT | Lama |
| | GGSLRLSCAASG | DV | QRELVAFIT | | NMVYLQMNSLKPEDTADY | SPL | QVTVSS | Glama |
| | | | | | LCRA | | | (9844) |
| 4QO1 | QVQLQESGGGLVQA | RTFS | MGWFRQAPGR | WSGT | TYYAESVKDRTTISRDNAK | HPQRGWGS | TYWGQGT | Lama |
| | GGSLRLSCAASE | TYA | EREFLAQIN | T | NTVYLEMNNLNADDTGIYF | TLGW | QVTVSS | Glama |
| | | | | | CAA | | | (9844) |
| 4W6X | QVQLQESGGGSVQA | YTYR | MGWFRQAPGK | SGGG | SYYADSVKGRFTISQDNAK | SSNSVCPPG | NDWGQG | Lama |
| | GGSLRLSCTASG | KYC | EREGVACIN | T | DTVFLRMNSLKPEDTAIYY | HVAWY | TQVTVSS | Glama |
| | | | | | CAL | | | (9844) |
| 4AQ1 | QVQLQESGGGLVQA | RTSS | MGWFRQAPGK | SKGG | TYYGASMKGRFTISRDNAK | SDKYNFDTS | GYWGQG | Lama |
| | GGSLRLSCAASG | AYA | EREFVAGIS | S | NTVYLQMNGLAPEDTAVY | HAGY | TQVTVSS | Glama |
| | | | | | YCAA | | | (9844) |
| 5SV3 | EVQLVESGGGLVQA | RTLG | VAWFRQAPGK | RSTII | TDYADSVRGRFSISADSAK | IANPVYATSR | GHWGQG | Lama |
| | GDSLRLSCTASG | DYG | EREFVSVIS | | NTVYLQMNSLKPEDTAVYY | NSDDY | TQVTVSS | Glama |
| | | | | | CAV | | | (9844) |
| 6H7J | QVQLQESGGGLVQA | SIFSI | MGWYRQAPGK | SGGS | TNYANSVKGRFTISRDNAA | KDYGAVLYE | DYWGQGT | Lama |
| | GGSLRLSCAASG | NT | QRELVAAIH | | NTVYLQMNSLKPEDTAVYY | Y | QVTVSS | Glama |
| | | | | | CNV | | | (9844) |
| 6Q6Z | QVQLQESGGGLVQA | FTFD | MGWFRRAPGK | RVTW | IDIADSVKGRFTISRDNAKN | SQQRLSRSD | DYWGQGT | Lama |
| | GGSLRLSCTASG | SYH | EREFVAAVS | L | TVYLEMNSLKPEDTAQYFC | VQY | QVTVSS | Glama |
| | | | | | AA | | | (9844) |
| 1QD0 | QVQLQESGGGLVQA | RAAS | MGWFRQVPGK | WSGK | TWYKDSVKGRFTISRDNAK | RPVRVADISL | DYWGQGT | Lama |
| | GGSLRLSCAASG | GHGH | EREFVAAIR | E | TTVYLQMNSLKGEDTAVYY | PVGF | QVTVSS | Glama |
| | | YG | | | CAA | | | (9844) |
| 1SJV | QVQLQESGGGLVQA | NTFS | MGWYRQAPGK | SRGI | TNYADFVKGRFTISRDNAK | HYFR | SYWGQGT | Lama |
| | GESLKLSCAASG | GGF | QRELVATIN | | KTVYLEMNSLEPEDTAVYY | | QVTVSS | Glama |
| | | | | | CYT | | | (9844) |
| 1U0Q | QVQLQESGGGLVQA | RTFS | VGWFRQAPGK | TRGG | TYYADSVKGRFTIAIDNAKN | RMPYSGDYR | DYWGQGT | Lama |
| | GGSLRLSCAASG | TYA | EREFVGYFG | R | TVYLQMNSLKLDDTAVYYC | SSGTY | QVTVSS | Glama |
| | | | | | AV | | | (9844) |
| 1G9E | QVQLQESGGGLVQA | RTGS | MGWFRQAPGK | WDSA | TYYASSVRGRFTISRDNAK | GEGGTW | DSWGQGT | Lama |
| | GGSLRLSCAASG | TYD | ERESVAAIN | R | KTVYLQMNSLKPEDTAVYT | | QVTVSS | Glama |
| | | | | | CGA | | | (9844) |
| 1I3V | QVQLQESGGGLVQA | DSIGT | IGWFRQAPGK | RNLV | TRYADSVKGRFAVSRDNA | KTTTWGGND | NYWGQGT | Lama |
| | GDSLKLSCEASG | YV | ERIYLATIG | GPSD | KNTVNLQMNSLKPEDTAVY | PNNW | QVTVSS | Glama |
| | | | | FY | YCAA | | | (9844) |
| 6QUP | QVQLVESGGGLVQP | SIFSI | MGWYRQAPGK | SGGT | THYADSVKGRFTISRDNAK | RVYFGDR | DYWGQGT | Lama |
| | GGSLRLSCAASG | DY | ERELVAIKT | | NTVYLQMNSLKPDDTAVYY | | QVTVSS | Glama |
| | | | | | CNA | | | (9844) |
| 6QX4 | QVQLVESGGGLVQA | SIFRI | MGWYRQAPGK | SGGS | TNYADSVKGRFTISRDNAK | DFSTGWAPY | DYWGQGT | Lama |
| | GGSLRLSCAASG | ND | QRELVAAIT | | KMVYLQMNSLKPEDTAVY | | QVTVSS | Glama |
| | | | | | YCHA | | | (9844) |
| 6S0Y | QVQLQESGGGLVQT | FTSD | IGWFRQAPGK | LSGG | TIYADSAKGRFTVSADNAK | ERYNVEGCG | AYWGKGT | Lama |
| | GGSLRLSCAFSG | DYV | GRQGVSCIR | G | KTVYLQMTRLKPEDTAVYY | YDV | QVTVSS | Glama |
| | | | | | CGA | | | (9844) |
| 6RUV | QVQLVESGGGLVQA | RTFTI | MGWFRQAPGK | RSGE | TDYADSVKGRFTISRDNNK | GRAILVHTTK | DHWGQG | Lama |
| | GGSLRLSCAASE | YA | EREFVAAIS | N | NTISLQMNSLKPEDTAVYY | KEY | TQVTVSS | Glama |
| | | | | | CAA | | | (9844) |
| 6EHG | QVQLVETGGGLVQA | SIFSL | MGWFRQAPGK | RSGG | TYYADSVKGRFTISRDNGK | GTGWSPQTD | NYWGQGT | Lama |
| | GGSLRLSCAASG | NA | EREFVATIN | R | NMVYLQMHSLKPEDTAIYY | NEY | QVTVSS | Glama |
| | | | | | CAA | | | (9844) |
| 6RTY | QVQLQESGGGLVQP | SGNSI | MGWYRQAPGK | SSGT | TNYADSVKGRFSISRDNAK | VLVRFGGLR | SYWGQGT | Lama |
| | GGSLRLSCAASG | NV | PRELVAEIT | | NTVPLQMNSLKPEDTAIYY | R | QVTVSS | Glama |
| | | | | | CSA | | | (9844) |
| 6RUL | QVQLVESGGALVQP | FPVN | MRWYRQAPGK | SAGD | SSYEDSVKGRFTISRDDAR | NVGFLPADLA | EYWGQGT | Lama |
| | GGSLRLSCAASG | RYS | EREWVAGMS | R | NTVYLQMNSLKPEDTAVYY | WFKRNTLNK | QVTVS | Glama |
| | | | | | CNV | PVIMGRHTW | | (9844) |
| | | | | | | ESIGRPLPGR | | |
| | | | | | | KNIILSSQPG | | |
| | | | | | | TDDRVTWVK | | |
| | | | | | | SVDEAIAACG | | |
| | | | | | | DVPEIMVIGG | | |
| | | | | | | GRVYEQFLP | | |
| | | | | | | KAQKLYLTHI | | |
| | | | | | | DAEVEGDTH | | |
| | | | | | | FPDYEPDDW | | |
| | | | | | | ESVFSEFHD | | |
| | | | | | | ADAQNSHSY | | |
| | | | | | | CFEILERRGG | | |
| | | | | | | GGGMISLIAA | | |
| | | | | | | LAVDRVIGME | | |
| | | | | | | NAMPWN | | |
| 6RVC | QVQLQESGGGLVQA | RTFS | MGWFRQAPGK | STGS | TYYADSVKGRFTISRDNAK | KVAGGSYYQ | DYWGQGT | Lama |
| | GDSLTLSCAASG | SYT | ERDFIAGIT | S | NTVYLQMNSLKPEDTADYY | KDKY | QVTVSS | Glama |
| | | | | | CAR | | | (9844) |
| 6YZ7 | QVQLVESGGGLMQA | RTFS | MGWFRQAPGK | WSGG | AYYADSVKGRFTISRDKAK | TENVRSLLSD | DYWGQGT | Lama |
| | GGSLRLSCAVSG | TAA | EREFVAAIR | S | NTVYLQMNSLKYEDTAVYY | YATWPY | QVTVSS | Glama |
| | | | | | CAR | | | (9844) |
| 6ZHD | QVQLVESGGGLMQA | RTFS | MGWFRQAPGK | WSGG | AYYADSVKGRFTISRDKAK | THYVSYLLSD | DYWGQGT | Lama |
| | GGSLRLSCAVSG | TAA | EREFVAAIR | S | NTVYLQMNSLKYEDTAVYY | YATWPY | QVTVSS | Glama |
| | | | | | CAQ | | | (9844) |
| 6QGW | QMQLVESGGGLVQA | RTFS | MGWFRQAPGK | RSGR | TSLADSVKGRFTISRDNGK | DPQWSRVRS | DYWGQGT | Lama |
| | GGSLTLSCAASG | DYD | AREFVARIS | M | RTVYLQMNSLKPEDTAVYY | GA | RVTVSA | Glama |
| | | | | | CAA | | | (9844) |
| 6QGY | QGQLVESGGGMVQ | RTFN | AAWFRQAPGK | RSGD | TYYTNSVKGRFTISRDSAK | KTGTWATMD | DYWGQGT | Lama |
| | AGGSLRLSCAASG | GWT | DREFVAAIS | Y | NNLYLQMDSLKPEDTAVYY | RRY | RVTVSA | Glama |
| | | | | | CAA | | | (9844) |
| 6QGX | QVQLVESGGGLVQA | RTFS | MAWFRRAPGK | ASAG | IFYTDSVKGRFTISRDHAKN | KTGTWATLD | DYWGQGT | Lama |
| | GGSLRLSCAASG | RYA | EREFVAAIS | T | TVSLQMNSLRPEDTAVYYC | RRY | RVTVSA | Glama |
| | | | | | AA | | | (9844) |
| 6B20 | QVQLVESGGGLVQA | SIFSI | MGWYRQAPGK | RGGR | TNYADSVKGRFTLSRDNAK | GRSR | GYWGQG | Lama |
| | GGSLRLSCAASG | NA | QRELVAAIT | | NTVYLQMNSLKPEDTAVYY | | TQVTVSS | Glama |
| | | | | | CNV | | | (9844) |
| 5O8F | QVQLQESGGGLVQA | HTFN | MGWFRQAPGK | WSGG | TSYADSVKDRFTISRDNAK | KGRYSGGLY | DYWGQGT | Lama |
| | GGSLRLSCAASG | YPI | EREFVGAIS | S | NTVYLEMNNLKPEDTAVYY | YPTNY | QVTVS | Glama |
| | | | | | CAA | | | 9844) |
| 6EZW | QVQLVQSGGGLVQA | RTFS | MGWFRQAPGK | GRGL | PDIADSVNGRFTISRDNAK | DLQYGSSWP | DYWGQGT | Lama |
| | GGSLRLSCAFSG | MYT | EREFVAANR | S | NTLYLQMDSLKPEDTAVYY | QRSSAEY | TVTVSS | Glama |
| | | | | | CAA | | | 9844) |
| 6H02 | QVQLVESGGGLVQA | RTGSI | MGWFRQAPGK | WTPG | TKYADSVKGRVAISRDIAK | CAYGTCYYG | EYWGQGT | Lama |
| | GASLRLSCAVSG | YT | EREVVARTT | S | NTLYLQMNNLKPEDTAVYY | DRAY | QVTVSS | Glama |
| | | | | | CAA | | | (9844) |
| 6H16 | EVQLQESGGGLVQA | RTFSI | IGWFRQAPGK | WSGG | TYYADSVKGRFTISRDNAK | ITYTRGIY | KYWGQGT | Lama |
| | GGSLRLSCAASG | YT | EREFVAEIT | S | NTVYLQMNSLKPEDTAVYY | | QVTVSS | Glama |
| | | | | | CAA | | | (9844) |
| 6H15 | VQLQESGGCLVQAG | STFST | IGWFRQAPGK | WDGG | TYYADSVKGRFTISRDNAK | RGRRYFDFT | DYWGQGT | Lama |
| | GSLRLSCAASG | YT | EREFVAAIH | Q | NTVYLQMNSLKPEDTAVYY | YSDVY | QVTVS | Glama |
| | | | | | CAA | | | (9844) |
| 6FYU | EVQLVESGGGLVQA | RTYA | MGWFRQAPGK | ALGTR | TYYSDSVKGRFTISRDNAK | QGQWRAAP | EFWGQGT | Lama |
| | GGSLKLSCAASG | | EREFVAHIN | | NTEYLEMNNLKPEDTAVYY | VAVAAEY | QVTVS | Glama |
| | | | | | CTA | | | (9844) |
| 6FYS | EVQLVESGGGLVQP | FTLEN | IGWFRQTPGS | KSGS | TYYTDSMRGRFTISRDNAE | TTAGGGLCW | SSWGQGT | Lama |
| | GGSLRLSCAATG | KA | EREGVLCIS | W | NTVYLQMDSLKPEDTAVYY | DGTTFSRLA | QVTVSS | Glama |
| | | | | | CAT | | | (9844) |
| 6GCI | QVQLVESGGGLVQA | ITFSS | MGWFRQAPGK | GSGS | TYYADSVKGRYTISRDNAK | RDDGRMYQ | EYWGQGT | Lama |
| | GDSLRLACAASG | YT | EREYIARIT | N | NTAYLQMNSLKPEDTANYY | GFYDF | QVTVSS | Glama |
| | | | | | CAA | | | (9844) |
| 5EUL | QVQLVETGGGLVQP | SIFNM | MGWYRQAPGK | TDDS | TMYPDSVKGRFTISRDNAK | QRTVMSQ | PYWGQGT | Vicugna |
| | GGSLRLSCGASG | YA | QREVVARIA | | NTVYLQMNSLKPEDTAVYY | | QVTVSS | Pacos |
| | | | | | CYY | | | (30538) |
| 6ITC | VALVESGGALVQPG | FPVN | MRWYRQAPGK | AGDR | SSYEDSVKGRFTISRDDAR | NVGF | EYWGQGT | Lama |
| | GSLRLSCAASG | RYS | EREWVAGMS | | NTVYLQMNSLKPEDTAVYY | | QVTVS | Glama |
| | | | | | CNV | | | (9844) |
| 5G5X | QVQLQESGGGLVQP | SGFN | MGWYRQAPGK | SFGS | TNYADSVKGRFTISRDNAK | GWGATPR | SYWGQGT | Lama |
| | GGSLRLSCAASG | NNA | QRELVAAIT | | NTVYLQMNSLKPEDTAVYY | | QVTVSS | Glama |
| | | | | | CTA | | | (9844) |
| 5O2U | DVQLQESGGGLVQA | SISRF | MGWWRQAPGK | KGFD | PVLADSVKGRFTISIDSAEN | ALDT | AYWGQGT | Lama |
| | GGSLRLSCAASG | NA | EREFVARIV | | TLALQMNRLKPEDTAVYYC | | QVTVSS | Glama |
| | | | | | FA | | | (9844) |
| 6C9W | VQLVESGGGLVQAG | GTFS | MGWFRQDLGK | WTGG | AYYGDSVKGRFTISRDNAK | QGTNGGGYS | NYWGQGT | Lama |
| | DSLRLSCAASG | TFN | EREFVAAIR | R | NTVYLQMNSLKPEDTAVYY | EATSY | QVTVSS | Glama |
| | | | | | CAR | | | (9844) |
| 5VAK | QVQLVESGGGLVQA | RTFS | GGWFRQAPGK | WSGG | KLYADSVKGRFTISRDNAK | RRINEVLTTA | DFWGQGT | Lama |
| | GGSLRLSCAASQ | PYV | EREFVAAIS | T | NTVYLQMNTLKREDTAVYY | PDY | QVTVSS | Glama |
| | | | | | CAA | | | (9844) |
| 6F2G | QVQLVESGGGVVQA | RTFS | MGWFRQAPGE | WSGS | TEYADSVKGRVTISRDNAK | KNGGAASNY | VYWGQGT | Lama |
| | GGSLRLSCAASG | SRA | GREFVATIS | Y | NTVYLQMNSLKPGDTAVY | PNDY | QVTVSS | Glama |
| | | | | | HCAA | | | (9844) |
| 6EQI | QVQLVESGGGLVQA | RPFS | LAWFRQAPGK | RGGS | TFYADSVSGRFTISGGSTK | SITGYGLVDS | TIWGQGT | Lama |
| | GGSLKLSCTTSG | TFD | EREFVSGLR | | NTLYLQMNSLKPEDTAIYY | ASHY | QVTVSS | Glama |
| | | | | | CAR | | | (9844) |
| 6NI2 | QVQLQESGGGLVQA | FFFDT | MAWYRRAPGK | AGGT | TTYADSVKDRFTISRDNAK | FVRS | LSWGQGT | Lama |
| | GGSLRLSCVVSG | VT | HRELVASAT | | NTVYLQMNSLKPEDTAVYY | | QVTVSS | Glama |
| | | | | | CNT | | | (9844) |
| 6O3C | QVQLQESGGGLVQA | YIFSS | MGWYRQAPGK | WGTI | TYYADSVKGRFTISRDNAK | QDLLYYSFP | AYWGQGT | Lama |
| | GGSLRLSCAASG | YA | EREFVATIG | | NTVYLQMNSLKPEDTAVYY | GDH | QVTVSS | Glama |
| | | | | | CAA | | | (9844) |
| 6OZ6 | DVQLQESGGGLVQT | RSFS | MAWFRQAPGK | RRGN | TAYADAVKGRFTISRDNAA | FRVAVTTYTS | NYWGQGT | Lama |
| | GGSLTLSCATSG | LYA | EREFVAGVS | | NTVYLQMTSLKPEDTAVYF | QQANEY | QVTVSS | Glama |
| | | | | | CAA | | | (9844) |
| 6GJQ | QVQLQESGGGLEQP | VIFGI | MGWYRQAPGK | SGGS | TNYADFVEGRFTISRDNAK | TVVVSRYGLT | DYWGQGT | Lama |
| | GGSLRLSCATSG | NA | QRELVATFT | | NTVYLQMNGLRPEDTAVY | Y | QVTVSS | Glama |
| | | | | | YCHA | | | (9844) |
| 6GKD | QVQLQESGGGLVQA | SIRSI | MGWYRQAPGK | RVGN | TDYADSVKGRFTISRDNAK | EITEQSRPFY | DYWGQGT | Lama |
| | GSSLRLACAATG | NN | QRGMVAIIT | | NTVYLQMNSLKPEDTATYY | LTD | QVTVSS | Glama |
| | | | | | CHA | | | (9844) |
| 6GJU | QVQLQESGGGLVQA | STFAI | MGWYRQAPGK | TGD | TRYADSVKGRFTISRDNAK | AVQVRDYR | NYWGQGT | Lama |
| | GGSLRLSCAASG | IA | QRELVAVIS | | NTVYLQMDSLRPEDTAVYY | | QVTVSS | Glama |
| | | | | | CNA | | | (9844) |
| 6GK4 | QVQLQESGGGLVQP | STSSI | MGWYRQAPGK | SGGD | TRYAEPVKGRFTISRDNAQ | NWGRTSV | NSWGQGT | Lama |
| | GGSLRLSCAASG | NA | QREPVAISS | | NKVYLQMNSLKPEDTAVYY | | QVTVSS | Glama |
| | | | | | CWL | | | (9844) |
| 6GJU | QVQLQESGGGLVQA | SIFRI | MGWYRQAPGK | SGGS | TDYADSVKGRFTISRDNAK | DVRTRWYAS | NYWGQGT | Lama |
| | GGSLRLSCAASG | DA | QRELVAHST | | NTVYLQMNSLKPEDTAVYY | N | QVTVSS | Glama |
| | | | | | CNA | | | (9844) |
| 6GKD | QVQLQESGGGLVQA | RAFS | MGWFRQAPGK | GNGS | RDYADSVKGRFTISRDNAK | SSTYYYTDPE | DYWGQGT | Lama |
| | GGSLRLSCTASG | WYV | EREFVATVS | R | NTVYLQMNSLKPEDTAVYY | KY | QVTVSS | Glama |
| | | | | | CAA | | | (9844) |
| 4S11 | QVQLQESGGGLVQA | RTFS | MGWFRQAPGK | RSGS | TRYADSAKGRFTISKDNAK | DLHRPYGPG | DTWGQGT | Lama |
| | GGSLRLSCAASG | SFV | EREFVASIS | V | NTVSLQMDNLNPDDTAVY | SQRTDDY | QVTVSS | Glama |
| | | | | | YCAA | | | (9844) |
| 6GS4 | QVQLQESGGGLVQA | RTFS | MGWFRQAPGK | WTGR | ADYPDSVKGRFTISRDNAK | KQYGSRADY | DYWGQGT | Lama |
| | GGSLRLSCAGSG | SYN | EREFVGGIS | S | NAVYLQMNSLKPEDTAVYY | PWDDY | QVTVSS | Glama |
| | | | | | CAA | | | (9844) |
| 5LMW | QVQLVESGGGLVQA | LTFN | MGWFRQAPGR | WSGD | TYGTDSVKGRFAISRDNAK | DRFLTRSVKY | DYWGQGT | Lama |
| | GDSLRLSCAASG | RYN | EREFVASIS | R | NTMYLQMNSLKPEDTAVY | | QVTVSS | Glama |
| | | | | | YCAA | | | (9844) |
| 6HJX | QVQLQESGGGLVQA | RIFST | MGWFRQAPGK | RIGGS | TVYADFVKGRFTLSRDNAK | RIGGSDRLAP | GYWGQG | Lama |
| | GGSLRLSCAASG | NV | EREFVATVG | | NMVYLQMNSLKPEDTAVY | ENY | TQVTVSS | Glama |
| | | | | | YCGA | | | (9844) |
| 6I53 | QVQLQESGGGLVQA | RTFT | MAWFRQAPGK | QGRI | QYYGDSVRGRFTISRDYAK | GAGFWGLRT | HYWGQGT | Lama |
| | GGSLRVSCAASG | AYI | EREFLAAMD | | NSVDLQLDGLRPEDTAVYY | ASSY | QVTVSS | Glama |
| | | | | | CAA | | | (9844) |
| 6I8H | QVQLQESGGGLVQA | RTFS | MAWFRQAPGK | SSGG | VVYRDSVKGRFTIARDNAA | KWLAADY | NYWGQGT | Lama |
| | GGSLRLSCAGSG | TYD | EREFVSSIS | N | NAVYLQMNSLKPEDTAVYY | | QVTVSS | Glama |
| | | | | | CAA | | | (9844) |
| 6I8G | QVQLQESGGGLVQA | HTAG | MAWFRQAPGK | WSDY | TDYANSVKNRFTISRDNAK | GWPVKVISS | INWGQGT | Lama |
| | GGSLRLSCATST | QYT | EREFVAVLR | S | NTVYLQMNSLKPEDTAVYY | ADEY | QVTVSS | Glama |
| | | | | | CAA | | | (9844) |
| 6N51 | QVQLVESGGGLVQA | RTFT | MGWFRQAPGK | SSGG | THYADSVKGRFTISRDNSK | AMYGSRWP | DYWGQGT | Lama |
| | GGSLRLSCAASG | SYA | ERESVAAIS | S | NTVYLQMNSLKPEDTAVYY | DWEY | QVTVSS | Glama |
| | | | | | CAA | | | (9844) |
| 5LZ0 | DVQLVESGGGLVQA | RTFS | MGWFRQAPGK | RSDN | TYYADSVKGRFTISRDSAK | TPYGSRYYL | DYWGQGT | Lama |
| | GGSLRVSCAASG | SYS | EREFVAAIS | S | NTVYLQMNSLKPEDTAVYY | RELREY | QVTVSS | Glama |
| | | | | | CAA | | | (9844) |
| 6XZU | QVQLVESGGGLVQA | STFS | MGWYRQAAGE | STGR | TNYIDSVKGRFTISRDNAKT | LGATTMIN | TKWGQGT | Lama |
| | GGSLRLSCVVSG | DYA | QRELVAAIY | | TVYLQMNSLKPEDTAVYYC | | QVTVSS | Glama |
| | | | | | NL | | | (9844) |
| 6SWR | QRQLVESGGGLVQP | SILYF | MGWYRQAPGK | SGDS | TNYADPVKGRFTISRDNAK | KEKGWSFSL | DYWGQGT | Lama |
| | GGSLRLSCAASG | NR | QRELVAAIT | | NTVYLQMNSLKPEDTAVYY | Y | PVTVVK | Glama |
| | | | | | CNA | | | (9844) |
| 6SSP | QVQLQESGGGLVQA | FTFD | IGWFRQAPGK | SSLGS | TYYADSVKGRITISRDNAK | GRDADPTIFA | DYWGQGT | Lama |
| | GGSLRLSCAASG | DYT | EREGVSLIS | | NTVYLQMNSLKPEDTAVYY | ILRSEYPF | QVTVSS | Glama |
| | | | | | CAA | | | (9844) |
| 6SSI | QVQLQESGGGLAQA | RNFFI | MNWYRQAPGK | RAGT | TTYADSVKGRFTISRDNAK | G | ASWGQGT | Lama |
| | GGSMRLSCIASG | NI | QRELVAQIT | | NTVYLQMSTLQSEDTAVYY | | QVTVSS | Glama |
| | | | | | CNV | | | (9844) |
| 6U52 | QVKLQQSGGGSVQE | AFFR | MGWYRRAPGN | RNGR | TIYAPSLKDRFTISRDDDNN | NVRTAVAGR | DYWGQGT | Lama |
| | GGSLRLSCASSG | AGP | ERELVAGIS | | ILYLQMSDLTPGDTAVYYC | N | QVTVSS | Glama |
| | | | | | NL | | | (9844) |
| 6U53 | KVQLQQSGGGSVTP | SISDF | MAWYRQAPGK | SAGA | LLYAEPVKGRFTISRDNAK | YAEA | IYWGQGT | Lama |
| | GGSLRLSCAASG | AA | ERDWVGTIF | | NTVYLQMNSLKPEDTAVYY | | QVTVSS | Glama |
| | | | | | CRL | | | (9844) |
| 6UKT | QVQLQESGGGLEQA | LIVSN | MGWFRQAPGK | WNGG | TYYTNSVKGRFTISRDNAK | TSRASVTTRV | GYWGQG | Lama |
| | GDSLRLSCAASG | YA | EREFVAYIN | V | NTVYLQMNSLKPEDTAVYY | ADF | TQVTVSS | Glama |
| | | | | | CAR | | | (9844) |
| 6UKT | QVQLQESGGGLVQP | IIFRS | MAWYRQAPGK | SFGD | AIYRDSVKGRFTISRDNAR | YPVN | SAWGQGT | Lama |
| | GGSLRLSCAASG | NG | EREWVASIT | | NAVSLQTNSLKTEDTAVYY | | QVTVSS | Glama |
| | | | | | CNT | | | (9844) |
| 6UKT | QVQLQESGGGLVQA | GIVHI | MGWFRQAPGK | SNGD | IRYADSVKGRFTLSRDNAK | FLRHTASASY | NYYGQGT | Lama |
| | GGSLRLSCAASG | SS | QRELVATSP | | NTVSLQMNSLEPEDTAVYY | N | QVTVSS | Glama |
| | | | | | CHS | | | (9844) |
| 6UKT | QVQLQESGGGLVQA | RTSD | MAWFRQAPGK | WNSA | TWYADFVKGRFTISRDNAK | RAYGFDTDS | SNWGQGT | Lama |
| | GGSLRLSCAASV | TDG | EREFVGGIR | | NTLYLQMNSLKPEDTALYY | RESAY | QVTVSS | Glama |
| | | | | | CAR | | | (9844) |
| 6VI4 | QVQLQESGGGLVQA | TIFRL | MGWYRRVSGN | SGGS | TKYGDSVKGRFTISRDNAK | EYRTGIWEEL | DGWGQG | Lama |
| | GESLRLSCAASG | YD | QRELVASIT | | NTVYLQMSSLKPEDTAVYY | L | TQVTVSS | Glama |
| | | | | | CNA | | | (9844) |
| 6WAQ | QVQLQESGGGLVQA | RTFS | MGWFRQAPGK | WSGG | TYYTDSVKGRFTISRDNAK | AGLGTVVSE | DYWGQGT | Lama |
| | GGSLRLSCAASG | EYA | EREFVATIS | S | NTVYLQMNSLKPDDTAVYY | WDYDY | QVTVSS | Glama |
| | | | | | CAA | | | (9844) |
| 6WAR | QVQLQESGGGSVQA | SIFSI | MDWYRQAPGK | SGGS | TNYGDFVKGRFTISRDNAK | EVGGWGPP | DYWGHGT | Lama |
| | GGSLRLSCVASG | NA | QRELVAGIT | | NTVYLQMDSLKPEDTAVYY | RP | QVTVSS | Glama |
| | | | | | CAA | | | (9844) |
| 4LHJ | QVQLVESGGGLVQA | SIVNF | MGWYRQAPGK | NEGS | SNYADSVKGRFTISGDNAK | TFGSRWPYA | DHWGQG | Vicugna |
| | GGSLRLSCAASG | ET | ERELVATIT | | NTVSLQMNSLKPEDTAVYY | HS | TQVTVS | Pacos |
| | | | | | CSA | | | (30538) |
| 4LGP | QVQLVETGGGLVQP | GTLE | IGWFRQAPGK | GEYG | TVYVDSVKGRFTASRDNA | GCYSWR | GPWGQG | Vicugna |
| | GGSLTLSCAGSG | HYA | EHEWLVCNR | S | KNTVYLQLNSLKPDDTGIY | | TQVTVS | Pacos |
| | | | | | YCVS | | | (30538) |
| 4LGS | QVQLVESGGGLVQA | GDFS | MAWFRQAPGK | WTGS | TYYLDSVKGRFTISRDNAK | STVFAEITGL | DYWGQGT | Vicugna |
| | GGSLSLSCAASG | RNA | EREFVASIN | G | NALYLQMNNLKPEDTAVYY | AGYQSGSY | QVTVSS | Pacos |
| | | | | | CAR | | | (30538) |
| 5L21 | QVQLVESGGGLAQP | FGTW | VNWYRQPPGK | RYPK | VTYLDSVKGRFTISRDNAK | G | EFWGQGT | Vicugna |
| | GGSLRLSCEASG | FRFD | SREFDELVA | SGI | KMAFLQMDNLKPEDTAVY | | QVTISS | Pacos |
| | | ENT | | | YCNV | | | (30538) |
| 5LHQ | QVQLQESGGGLVQP | FTLG | IGWFRRAPGK | SSGG | TNYADSVKGRFTISRDNAK | EWVPPGYGA | EYWGKGT | Vicugna |
| | GGSLRLSCAASG | YYA | EREGVSCIS | S | NTVDLQMNSLKPEDTAIYY | TVQALCNNA | QVTVSS | Pacos |
| | | | | | CAA | GYGM | | (30538) |
| 5GXB | QVQLVESGGRLVQA | RTFTT | MGWFRQAPGK | WSGG | TYYADSVKGRFTISRDNAK | AARPSYSGD | DYWGQGT | Vicugna |
| | GDSLRLSCAASG | YL | EREFVAAIR | S | NTVYLQMNSLKLEDTAVYY | YGYTEALRY | QVTVSS | Pacos |
| | | | | | CAA | | | (30538) |
| 5HVH | QVQLQESGGGLVQA | SIFSP | MGWYRQAPGK | NVG | STYADSVKGRFTVSRDNA | WGQDGWLG | DYWGQGT | Vicugna |
| | GGSLRLSCAASG | NA | ERELVAART | | KNTVYLQMNSLKPEDTAVY | QY | QVTVSS | Pacos |
| | | | | | YCNA | | | (30538) |
| 5HVH | QVQLQESGGGLVQP | SIFSG | MGWYRQAPGK | SGGS | TDYADSVKGRFTISRDNAK | DPRPWGYDV | DYWGQGT | Vicugna |
| | GGSLRLSCAASG | NA | QRELVAAIT | | NTVYLQMNSLKPEDTAVYY | TDY | QVTVSS | Pacos |
| | | | | | CHV | | | (30538) |
| 4YGA | QVQLVETGGGLVQP | FTLD | VGWFRQVPGK | ANGV | LDYADSIKGRFTISRDNAKN | TREFCSAYVF | EHWGQGT | Vicugna |
| | GESLRLSCVASG | HSA | EREKLLCIN | S | TVYLQMNDLKPEDTATYSC | LY | QVTVSS | Pacos |
| | | | | | AA | | | (30538) |
| 5NBM | QVQLVESGGGSVQA | RTFT | MGWFRQAPGK | RFGF | LYYSDSVKGRFTISRDNAK | RNPRATAED | DYWGQGT | Vicugna |
| | GDSLRLSCTASG | NYG | EREFVAAIG | P | TTVYLQMHNLKPEDTAIYF | VSAY | QVTVSS | Pacos |
| | | | | | CAA | | | (30538) |
| 5O02 | QVQLQESGGGLVQP | FTFSS | MRWYRQAPGK | SAGG | THYADSVKERFTISRDNAK | RRDYGDSWF | GYWGQG | Vicugna |
| | GGSLRLFCAASG | YA | ERELVAAIT | S | NTMYLQMNSLKPEDTAVY | TAGG | TQVTVSS | Pacos |
| | | | | | YCNA | | | (30538) |
| 5J1S | QVQLVETGGGLVQA | NIFSF | MGWYRQAPGK | SGDT | TTYADSVQGRFTISRDNAK | RRNPINGPYY | AYWGQGT | Vicugna |
| | GGSLRLSCAASG | NV | QRELVAAIT | | NAVYLQMNSLTPEDTAVYF | TT | QVTVSS | Pacos |
| | | | | | CNA | | | (30538) |
| 5J57 | VQLAETGGGLVEPG | FRLQ | AGWFRQAPGK | AGGG | TYYTGSVQGRFTISRDNAK | DLEYSQIMPS | GVRGQGT | Vicugna |
| | GSLRLSCAAPE | YYT | EREWVACIS | V | RTVYLQMDSLKPEDTAVYS | CRGSY | QVTVSS | Pacos |
| | | | | | CAA | | | (30538) |
| 5J56 | QVQLVESGGGLVQP | FSGF | IGWFRQAPGK | SSSD | TSYSDSVKGRFTISRDNAK | RLGGWASFS | DYWGQGT | Vicugna |
| | GGSLRLSCVASE | TLDY | EREGLSSIS | GF | NTVYLQMNSLKPEDTAVYY | PQEY | QVTVSS | Pacos |
| | | YA | | | CAA | | | (30538) |
| 5IVN | QVQLVESGGGLVQP | FTLD | IGWFRQAPGK | NSDD | TYYADSVKGRFTIFMNNAK | ARGCKRGRY | DFWGQGT | Vicugna |
| | GGSLTLSCTASG | HYD | EREGVSCIN | D | DTVYLQMNSLKPEDTAIYY | EY | QVTVSS | Pacos |
| | | | | | CAE | | | (30538) |
| 4JVP | EVQLQASGGGLVQP | FTDD | IGWFRQAPGK | NFDG | TYYADSVKSRFTMSRDNA | DKGLCSWLR | GSWGQG | Vicugna |
| | GGSLRLSCTASG | YYA | EREGVSCIT | G | KNTVYLQMNSLKPEDTAVY | AGGKVTF | TQVTVSS | Pacos |
| | | | | | YCAA | | | (30538) |
| 2XXM | QVQLVESGGGLVQA | SFFM | MAWYRQAPGK | GGDM | TVYDDSVKGRFTITRDDDK | SG | SSWGQGT | Vicugna |
| | GGSLRLSCAASG | SNV | ARELIAAIR | S | NILYLQMNDLKPEDTAMYY | | QVTVSS | Pacos |
| | | | | | CKA | | | (30538) |
| 4TVS | QVQLVESGGGLVQA | RTLS | VGWFRQAPGL | RSGG | THYADSVKGRFTISRDNAK | TFTPDGSWY | DYWGQGT | Vicugna |
| | GGSLRLSCAASG | SYA | EREFVATIS | S | NTVYLQMNSLKPEDTAVYY | YTRGSSY | QVTVSS | Pacos |
| | | | | | CAA | | | (30538) |
| 4XT1 | QVQLVESGGGLVRP | SIFTIY | MGWYRQAPGK | FGGD | TNYADSVKGRFTISRDNAK | EETIVEEA | DYWGQGT | Vicugna |
| | GGSLRLSCAASG | A | QRELVARIT | | NAVYLQMNSLKPEDTAVYY | | QVTVSS | Pacos |
| | | | | | CNA | | | (30538) |
| 6QV2 | QGQLVESGGGLVQP | FTLDY | IGWFRQAPGK | NSGG | TKYADSVKGRFTISRDKAK | DRGYSEYDL | DYWGKGT | Vicugna |
| | GGSLRLSCAASG | YA | EREGVSCIS | S | NTVYLQMNSLKPEDTGVY | PCDLVIYGM | PVTVSA | Pacos |
| | | | | | YCAA | | | (30538) |
| 6QV1 | QGQLVESGGGLVQA | RDISF | VGWFRQAPGK | SLRK | INYADSVKGRFTISRDDAK | SLPGL | PYWGQGT | Vicugna |
| | GGSLTLSCAASV | FA | QRELVAQMT | | NTVSLQMNSLKPEDTAVYY | | PVTVSA | Pacos |
| | | | | | CHA | | | (30538) |
| 6RAK | QLQLVESGGGLVQP | SALD | IGWFRQAPGK | KITGN | TAYADSVKGRFTISRDNAK | VTAVLLPGRC | KYWGQGT | Vicugna |
| | GDSLRLSCAVSG | YNA | EREGVACIS | | NTVHLQMNSLKPEDTAVYY | VPG | PVTVSS | Pacos |
| | | | | | CAT | | | (30538) |
| 6IBB | EVQLVESGGGLVQP | YTLA | IGWFRQAPGK | SGGS | TVYSESVKDRFTISRDNAK | DPFGERLCID | ETWGQGT | Vicugna |
| | GGSLRLSCEASG | NYA | EREGVSCIS | | KIVYLQMNSLOPEDTAVYY | PNTFAGYL | QVTVSS | Pacos |
| | | | | | CAA | | | (30538) |
| 5UKB | QVQLVESGGGLVQA | RPFR | LGWFRQAPGK | WGGV | TNYADSVKGRFTISRDDAK | FDSTGSPIVR | EYWGQGT | Vicugna |
| | GGSLTLSCVASG | NYR | EREFVAAIN | I | NTVYLQMNSLKPEETAVYY | DNY | QVTVSS | Pacos |
| | | | | | CAA | | | (30538) |
| 5UK4 | QVQLVETGGGLVQT | RTFS | MGWFRQAPGK | WRND | TTYADSVKGRFTISRDNAS | TKAYNGGET | YYWGQGT | Vicugna |
| | GGSLRLSCKASG | NSI | ERDFVAKIS | Y | NMVYLLMNNLKPEDTAVYY | SGRGF | QVTVSS | Pacos |
| | | | | | CAA | | | (30538) |
| 6IYN | QLQLVESGGGLVQA | RTFS | MGWFRQAPGK | WSGR | TYYADSVKGRFTISRDNAK | GKWDGSYY | DYWGQGT | Vicugna |
| | GGSLRLSCAASG | SYA | EREFVAVVN | R | NTVYLQMNSLKPEDTAVYN | GAP | QVTVSS | Pacos |
| | | | | | CAA | | | (30538) |
| 5VXK | QLQLVESGGGLVQP | FTLD | IAWFRQAPGK | IDGNT | QSYSDSVKGRFTISRDTAN | DRYTSVRQM | RVWGQGT | Vicugna |
| | GGSLRLSCAASG | DQP | EREGVSCIS | | NRVHLQMNNLKPEDTAVY | CTMIEGLH | QVTVSS | Pacos |
| | | | | | YCAA | | | (30538) |
| 5VXM | QVQLVESGGGLVQA | LEMQ | IGWFRQAPGK | DDGS | TRYADSVKGRFTISRDNAK | KSVWFCSVIR | NSWGQGT | Vicugna |
| | GGSLRLSCAVSG | SHA | EREGVSCIN | T | NTVYLQMNSLKPEDTAVYF | SHEF | QVTVSS | Pacos |
| | | | | | CAA | | | (30538) |
| 6CWK | QVQLAESGGGLVQA | RDFS | LAWFRQAPGK | CSGG | TYYADSMQGRFTISRDNAK | STTYCSATTY | DFWGQGT | Vicugna |
| | GGSLKLSCAASG | MYM | EREFVAAIM | GGG | KTVALQMNSLKPEDTAVYY | SSDRLY | QVTVSS | Pacos |
| | | | | | CAA | | | (30538) |
| 5TD8 | QVQLVESGGGLVHP | RTGS | VAWFRQAPGK | AVGLV | RNYADSVLGRFSISRDFAK | RYYSGTYSS | DYWGQGT | Vicugna |
| | GGSLRLSCAASG | RHA | ERDFVASIN | | NEVYLQMNSLEPEDTAVYY | TYDRDDY | QVTVSS | Pacos |
| | | | | | CAA | | | (30538) |
| 6OCD | QLVETGGGLVQSGG | FTLD | IGWFRQAPGK | SSDG | TYYADSVKGRFTISRDNAK | TKYGSSCPIR | DYWGQGT | Vicugna |
| | SLRLSCAASG | NYN | EYGGVSCIS | S | NTVYLQMNNLKPEDTDVY | PY | QVTVSS | Pacos |
| | | | | | YCAA | | | (30538) |
| 6H71 | QVQLQESGGGLVQA | RMFSI | MGWYRQAPGK | EAGT | TTYADSVRGRFTIARDNAK | YIQLDSTIWF | AYWGQGT | Vicugna |
| | GGSLRLSCAASG | NS | ERELVATIS | | NTVYLQMNSLNPEDTAVYY | R | QVTVSS | Pacos |
| | | | | | CNA | | | (30538) |
| 6GWP | QVQLVESGGGLVQP | FTFRT | MQWYRQSPGT | NIGGV | TDYGDSVKGRFTISRDNAK | VRLPQ | RYWGRGT | Vicugna |
| | GGRLRLSCAASG | YA | ERELVAAIS | | TTVYLEMNSLKPEDTATYY | | QVTVSS | Pacos |
| | | | | | CSA | | | (30538) |
| 6GWP | QVQLVESGGGLVQA | FTFD | IAWFRQAPGK | SSDG | AYYADSVKGRFTISSDNAK | VWARVCRNP | DYWGQGT | Vicugna |
| | GGSLRLSCAASG | DYS | EREGVSCIS | S | NTVYLQMNSLKPEDTAVYY | Y | QVTVSS | Pacos |
| | | | | | CAA | | | (30538) |
| 6I2G | EVQLQESGGGLVQP | VTISA | MGWYRQAPGE | ERGN | AMYRESVQGRFTVTRDFT | LEDRVDSFH | DYWGQGT | Vicugna |
| | GGSLRLSCTASG | LNAM | RRVMVAAVS | | NKMVSLQMDNLKPEDTAV | | QVTVSS | Pacos |
| | | A | | | YYCHV | | | (30538) |
| 5E1H | VQLAETGGGLVQAG | TTFSK | MAWFRQAPGK | WNAV | TNYADSVKGRFTVSRDNA | SSIYSDISGA | NYWGQGT | Vicugna |
| | GSLRLSCAASG | NA | EREFVAGIN | S | KNTVYLQMNSLKPEDTAVY | ATVWATSY | QVTVSS | Pacos |
| | | | | | YCAG | | | (30538) |
| 6ZRV | QVQLVESGGGLVQP | FSLD | IGWFRQAPGK | SSDG | TYYTDSVEGRFTISRDNAK | DYGSSWCTF | DYWGQGT | Vicugna |
| | GGSLRLSCAASG | NYA | EREGVSCIS | S | NTVYLQMNSLKPDDTAVYY | NGM | QVTVSS | Pacos |
| | | | | | CAA | | | (30538) |
| 6XW4 | QVQLQESGGGLVQA | RTFR | MGWFRQAPGK | WSAAI | TNYADSVKGRFTILRDKGM | TYSTIAPRTS | DFWGQGT | Vicugna |
| | GGSLRLSCAKSG | AYA | EREFVAAID | | NTAYLQMNSLEPEDTAVYY | Y | QVTVSS | Pacos |
| | | | | | CAA | | | (30538) |
| 6XW6 | QVQLQESGGGLVQA | RTISS | MGWFRQAPGK | GNGG | TYYLDSVKGRFTTSRDNAK | RSRFSAMHL | DDWGQG | Vicugna |
| | GDSLRVSCAASG | SP | EREFVAAIS | N | NTVYLQLNNLKPEDTAIYY | AYRRLVDY | TQVTVSS | Pacos |
| | | | | | CAA | | | (30538) |
| 6XW5 | QVQLQESGGGLVQA | RTFSL | MGWFRQAPGE | RAAY | TYYADSVKGRFTISRDNAK | KGQGGTW | DYWGQGT | Vicugna |
| | GGSLRLSCAASG | TT | DRAFVTSIS | | NMVSLQMNSLKPEDTAVY | | QVTVSS | Pacos |
| | | | | | VCAG | | | (30538) |
| 6XW7 | QVQLQESGGGLVEA | LTFSR | MGWFRQAPGK | RSGG | PNYADSVKGRFTISRDNAK | RGSVYY | DVWGQGT | Vicugna |
| | GGSLRLSCLGSG | YA | EREFVASIT | S | NTVYLQMSSLKPEDTAVYY | | QVTVSS | Pacos |
| | | | | | CAG | | | (30538) |
| 5OMM | QVQLQESGGGLVQS | NINS | VGWYRQAPGN | DDGS | TDYVDSVKGRFTISRDIAEN | TIVVFTTPM | HYWGKGT | Vicugna |
| | GGSLRLSCAASR | MHV | QRELVASIT | | TVYLQMNSLNPEDTAVYYC | | QVTVSS | Pacos |
| | | | | | KG | | | (30538) |
| 6UHT | QVQLVESGGGLVQA | LTYDL | IGWVRQAPGK | STDG | TYYADSVKGRFTISRNNAK | APLAGRYCP | GYWGQG | Vicugna |
| | GGSLRLSCAASI | DYYY | EREGVSCIS | A | NTVYLQMNNLKPEDTAIYY | ASHEY | TQVTVSS | Pacos |
| | | | | | CAA | | | (30538) |
| 6UL6 | QVQLVESGGGLVQV | SDISG | MGWYRQAPGK | SGGS | TDYAGSVKGRFTISRDNAK | YGSG | DYWGQGT | Vicugna |
| | GGSLRLSCVVSG | IA | RREMVADIF | | KTSYLQMNNVKPEDTGVY | | QVTVSS | Pacos |
| | | | | | YCRL | | | (30538) |
| 6UL6 | QLQLVESGGGLVQP | SDFN | MGWYRQVPGK | TEGK | TNYGGSVKGRFTISRDNAK | DWKMGAWT | DYWGKGT | Vicugna |
| | GGSLRLSCVVSG | TYI | PRELVADIT | | NTVYLQMFGLKPEDAGNY | AGDYGI | LVTVSS | Pacos |
| | | | | | VCNA | | | (30538) |
| 6UI1 | QVQLVESGGGLVHP | SLPST | VGWYRQAPGK | LR | INYADSVKGRFTISRDNAK | EYT | HYWGKGT | Vicugna |
| | GGSLRLSCAPSA | PFNP | QREMVASIG | | NTVDLQMDSLRPEDSATY | | LVTVSS | Pacos |
| | | FNNM | | | YCHI | | | (30538) |
| 1SHM | DVQLQESGGGLVQA | ATGS | MGWFRQAPGK | WGSA | TYYASSVRGRFTISRDNAK | GRIGRSVFNL | TWWGQG | Lama |
| | GGSLRLSCAASG | TYD | ERESVAAIN | G | KTVYLQMNSLKPEDTAVYT | RRESWV | TQVTVSS | Glama |
| | | | | | CGA | | | (9844) |
| 1SJX | QVQLQESGGGLVQA | NIFRI | MGWYRQAPGT | SGGS | TKYADSVKGRFTISKDNAK | EDRHRIGTV | GYWGQG | Lama |
| | GGSLRLSCQASG | ND | QRELVAAIT | | NTVYLQMNSLKPEDTAVYY | | TQVTVSS | Glama |
| | | | | | CAA | | | (9844) |
| 3K7U | QVQLQESGGGLVQA | RTFS | MGWFRQAPGK | WTGG | LFYADSVNGRFTISRDNAK | RPQGDYVTA | DYWGQGT | Lama |
| | GGSLTLSCAASG | NNA | EREFVAAIS | L | RTVTLQMNSLKPEDTAVYY | HY | QVTVSS | Glama |
| | | | | | CAA | | | (9844) |
| 3K80 | QVQLQESGGGLVQA | RAFS | MGWFRQAPGK | RSGG | TQYAESLKGRFAISRDNAK | DLYGLGSHM | DSWGQGT | Lama |
| | GDSLRLSCVASG | SYG | ERAFVAAIS | L | NTVYLQMGSLKPEDTAVYY | ENEY | QVTVSS | Glama |
| | | | | | CAG | | | (9844) |
| 4B41 | EVQLVESGGGLVQP | SIISN | MGWYRQAPGK | SGGR | TTYADSVKGRFTISRDNAK | ASLVRGPL | DHWGQG | Lama |
| | GGSLRLSCAASR | NA | QRELVARIS | | TTVYLQMNSLKPEDTAVYY | | TQVTVSS | Glama |
| | | | | | CNA | | | (9844 |
| 4B5E | EVQLVESGGGLVQA | RSFS | MGWFRQAPGK | LNGG | TYSADSVKGRFTISRDNDK | REGDVGLVS | PYWGQGT | Lama |
| | GGSLRLSCAASG | RDA | ERDVVAAIN | R | NTVYLQMSNLKPEDTAVYY | YKRSSNY | QVTVSS | Glama |
| | | | | | CAA | | | (9844) |
| 4LGR | VQLVESGGGLVQPG | SIASI | TCWYRQGTGK | SGGN | TYYADSVKGRFTISRDNAK | DEAGIGGFN | DYWGQGT | Vicugna |
| | GSLRLHCAASG | YR | QRELVAAIT | | NTIDLQMNSLKPEDTAVYY | | QVTVSS | Pacos |
| | | | | | CNA | | | (30538) |
| 4LHQ | VQLVETGGGTVQTG | GSFS | MGWFRQAPGK | WSAS | TYYRDSVKGRFTVSRDNA | SSVYAEMPY | NYWGQGT | Vicugna |
| | GSLRLSCSASG | RNA | EREFVAAIN | S | KNTVYLHLNSLKLEDTAAY | ADSVKATSY | QVTVSS | Pacos |
| | | | | | YCAG | | | (30538) |
| 4M3K | QVQLQESGGGLVQP | SISSIT | MGWYRQDPGK | SVGD | TTYAGSVKGRFTISRDNAK | FMSTNSGRT | SFWGQGT | Lama |
| | GGSLRLSCAASG | T | GRELVALIN | | NTVYLEMSSLKPEDTAVYY | G | QVTVSS | Glama |
| | | | | | CNA | | | (9844) |
| 4NC2 | QVQLVESGGGLVQA | LTFSR | MGWFRQAPGK | WGGT | PNYADSVKGRFTISRDNSK | GLGWDSRYS | NYWGQGT | Lama |
| | GGSLRLSCAASG | YV | EREFVAAIT | | NTQYLQMNSLKPEDTAVY | QSY | QVTVSS | Glama |
| | | | | | YCAA | | | (9844) |
| 4P2C | QVQLQESGGGLVQA | SIFRL | MGWYRQAPGK | SYGD | TNYRDSVKGRFTISRDNAK | NIEAGTYYGP | DYWGQGT | Lama |
| | GGSLRLSCAVSG | S | QREFVASIT | | NTVYLQMNSLKPEDTAVYY | GR | QVTVSS | Glama |
| | | | | | CNA | | | (9844) |
| 4Y7M | QVQLVESGGGLVQA | FTFE | IGWFRQAPGK | NLDG | TYYPDSVKGRFTASSDKAK | VNAQGIYCTD | DYWGKGT | Lama |
| | GGSLRLSCAASG | DYA | EREGVSCIS | S | NMVYLQMNSLKPEDTAVY | YIIGPYGM | QVTVSS | Glama |
| | | | | | YCAA | | | (9844) |
| 4QLR | DVQLVESGGGLVHP | RTFS | LGWFRQAPGK | WSGG | TYYADSVQGRFTISRDNDK | DKYYTGPGG | DYWGRGT | Lama |
| | GGSLRLSCAASG | DYA | DREFVAAIS | S | NSVYLQMNSLKPEDTAVYY | ESVY | QVTVSS | Glama |
| | | | | | CAA | | | (9844) |
| 6APQ | KVQLQESGGGLVQA | GTFSI | LGWYRRAPGK | SGGI | TRYADSVKGRFTISRDNGK | RNWGGGLD | VYWGQGT | Lama |
| | GGSLRLSCAASG | NT | EREFVARIS | | NTVYLDMNSLKPEDTAVYY | | QVTVSS | Glama |
| | | | | | CMY | | | (9844) |
| 4W2Q | KVQLQESGGGLVQV | FTFR | MGWYRRAPGK | TTGT | ADYGDFVKGRFTISRDNAE | DPYGMESL | RYWGQGT | Lama |
| | GGSLRLSCKASG | SSA | QRELVASLT | | NTVDLHMNSLKPEDTAVYY | | QVTVSS | Glama |
| | | | | | CHE | | | (9844) |
| 4W6W | QVQLQESGGGSVQA | YTSG | MGWFRQAPGK | TSGI | VNYADSVKGRFTISQDSAK | GPFVYGRGC | SYWGQGT | Lama |
| | GGSLRLSCAASG | RDS | EREGVACID | | KTLYLEMNSLKPEDTALYS | LGQAFY | QVTVSS | Glama |
| | | | | | CAT | | | (9844) |
| 4W6Y | QVQLQESGGGSVQA | YTYS | MAWFRQVPGK | TRGGI | TYYADSVKGRFTISRDNAK | VREATYSDN | DYWGQGT | Lama |
| | GGSLRLSCAASG | SNC | EREGVASIN | | NTVSLQMNSLKPEDTATYY | RCSVRSYTY | QVTVSS | Glama |
| | | | | | CAA | | | (9844) |
| 4WEM | QVQLQESGGGLVQA | NVDRI | MGWFRQAPGK | EGGI | LNYGDFVKGRFTISRDNAK | SHWGTLLIKG | EHWGKGT | Lama |
| | GGSLRLSCEASG | DA | QREFVGYIS | | NTVYLQMSNLKSEDTGVYF | I | QVTVSS | Glama |
| | | | | | CAA | | | (9844) |
| 4WEN | QVQLQESGGGLVQP | SISSI | MGWYRQAPGS | NTGV | TEFADSVKGRFTISRDNAK | TDWGTLLIKG | DHWGKGT | Lama |
| | GGSLRLSCTASG | NA | KREFVAHIT | | TTVDLQMNSLKPEDTAVYY | I | QVTVSS | Glama |
| | | | | | CAA | | | (9844) |
| 4WEU | QVQLQESGGGLVQA | LTFDT | MGWFRQAPGK | WTGIS | TYYADIAKGRFTISRDNAKN | QKSLNVPAP | DYWGQGT | Lama |
| | GGSLRLSCAASG | YA | KREYVAAIS | | TLYLQMDSLKPEDTAVYYC | W | QVTVSS | Glama |
| | | | | | AA | | | (9844) |
| 5O04 | QVQLQESGGGLVQP | SIFSIY | MGWYRQAPGK | SGGG | TNYADSVKGRFTISGDNAK | EDYSAYAPP | GSRGRGT | Vicugna |
| | GGSLRLSCAASG | A | QRELVASIS | | NTVYLQMNSLKPEDTAVYY | S | QVTVSS | Pacos |
| | | | | | CKR | | | (30538) |
| 4X7F | DVQLVESGGGLVQP | SILSF | MAWYRQGPGE | REGS | TDYADSVKGRFTISRDNAK | GIS | NPWGQGT | Vicugna |
| | GGSLRLSCAASE | NH | QRELVAVIT | | NMVYLLMSNLRPEDTAVYY | | QVTVSS | Pacos |
| | | | | | CNR | | | (30538) |
| 4ZG1 | QVQLQESGGGLVQA | STFGI | MGWYRQAPGK | SGGS | TDYADSVKGRFTISRDNAK | RVGITML | AHWGQGT | Lama |
| | GGSLRLSCAASG | RT | QRDLVAIIS | | NTVYLQMDSLKPEDTAIYY | | QVTVSS | Glama |
| | | | | | CNA | | | (9844) |
| 5BOP | QVQLQESGGGLVQA | GTFS | MGWFRQAPGK | WTGA | TYYADSVRGRFTISRDNAK | RRWLGGSYF | DFWGQGT | Lama |
| | GGSLRLSCAASG | TYG | EREFVAASS | N | NTVYLEMNSLKPEDTAVYY | DPGNY | QVTVSS | Glama |
| | | | | | CAA | | | (9844) |
| 5BOZ | QVQLVESGGGLVQA | RTFS | MGWFRQAPGK | WADG | TLYGDSVKGRFTVSRDNV | NKFVRGLVA | DYWGQGT | Vicugna |
| | GGSLRLSCAASG | RSS | EREFVASIV | T | KNMVYLQMNNLKPEDTAL | VRAIDY | QVTVS | Pacos |
| | | | | | YYCAD | | | (30538) |
| 5E0Q | QVQLVESGGGPVEA | RSFS | MAWFRQAPGK | WSSG | TSIADSVKGRFTMSRDPAK | SNRGSLYTL | EDWGQGT | Vicugna |
| | GGSLRLSCAASG | NSV | EREFLSVLN | R | ITVYLQMNGLKPEDTAVYY | DNQNRY | QVTVSS | Pacos |
| | | | | | CAA | | | (30538) |
| 5F1K | DVQLQESGGGLVQA | RTFR | MAWFRQAPGK | WVGA | TLYADFAKGRFTISRDNAK | GRGIVAGRIP | ADWGQGT | Lama |
| | GGSLRLSCTGSG | NYP | EREFVAGIT | S | NTVYLQMNSLKPEDTAVYS | AEY | QVTVSS | Glama |
| | | | | | CAA | | | (9844) |
| 5F21 | DVQLQESGGGSVQA | LLFRL | MGWYRQAPGK | VGGK | DSVQGRFIITRDNTGDNTK | ASPAVGA | DTWGQGT | Lama |
| | GGSLTLSCTASG | AS | ERELIATIT | TNYK | STVTLQMNRLKPEDTAVYY | | RVTVSS | Glama |
| | | | | | CNT | | | (9844) |
| 6EY6 | QVQLVESGGGLVQA | RTFS | MGWFRQAPGK | WSGG | IHYADSVKGRFTISRDNAK | GFAGYGSFT | DYWGQGT | Lama |
| | GGSLRLSCAASG | SYV | EREFVTAIS | S | NTVYLQMNSLKPEDTAVYT | SRSARDSDK | KVTVSS | Glama |
| | | | | | CVA | Y | | (9844) |
| 5JA8 | QVQLQESGGGLVQP | FTLDY | IGWFRQAPGK | SSGG | TNYADSVKGRFTVSRDNA | DFACPLIREY | DYWGQGT | Lama |
| | GGSLRLSCAASG | YA | EREGVSCIS | T | KNTVYLQMNSLKPEDTAVY | | QVTVSS | Glama |
| | | | | | YCVA | | | (9844) |
| 5JA9 | QVQLQESGGGLVQP | FTFS | MRWYRQAPGE | SVGG | TNYADSVKGRFTISRDNGK | RLSLIS | DSWGQGT | Lama |
| | GGSLRLSCAASG | NYA | EREFVAFIS | S | NTLYLQMNSLKPEDTAVYF | | QVTVSS | Glama |
| | | | | | CVA | | | (9844) |
| 5LHR | QVQLQESGGGLVQA | RTFS | MGWFRQAPGK | WSGG | TNYADSVKGRFTISRDNAK | DLASSRDVS | WYWGQG | Vicugna |
| | GGSLRLSCAASG | SYV | EREFVAAIS | S | NTVYLQMNSLKPEDTAVYY | S | TQVTVSS | Pacos |
| | | | | | CAA | | | (30538) |
| 5LMJ | QVQLVESGGGLVQA | RTFS | MAWFRQAPGK | TKGG | TWYHDSVKGRFTISRDNAK | GAVTYYSAR | DYWGQGT | Lama |
| | GGSLRLSCTASG | DYD | ERDRVSAIS | S | NTVYLQMNSLKPEDTAVYY | YEY | QVTVSS | Glama |
| | | | | | CAA | | | (9844) |
| 5M7Q | QVQLQESGGGSVQA | NTGR | MGWFRQAPGK | RLGG | TSYAGPVKGRFTISQDNAK | DSRPIYSGT | RYWGQGT | Camelus |
| | GGSLRLSCAVSE | | EREKVAIIT | Y | NTVYLLMNSLKPEDTAIYY | W | QVTVSS | Dromedarius |
| | | | | | CAA | | | (9838) |
| 5MJE | QVQLQESGGGLVQP | DRRTI | MGWFRQAPGN | SSGV | TTYVDSVKGRFSISRDSAE | TCYEESRRP | TYWGQGT | Lama |
| | GGSLRLSCVVSG | YT | QGELVATMT | | DSAKNTVSLQMNSLKPEDT | LGSRN | QVTVSS | Glama |
| | | | | | AFY | | | (9844) |
| 5O03 | QVQLQESGGGLVQP | FTLG | IGWFRQAPGK | GSGG | ANYAASVKGRFTISRDNAK | DLSSLTTVQA | DYWGLGT | Lama |
| | GGSLRLSCAASG | YYP | GLEGVSCIS | S | NTVYLQMNSLKPEDTAIYY | MCVIPRPGFS | QVTVSS | Glama |
| | | | | | CAA | AKAY | | (9844) |
| 5O04 | QVQLQESGGGLVQP | IIFFM | VGWYRQAPEK | SDVS | TKYADSVKGRFTISRDNAK | RRASA | DYWGQGT | Vicugna |
| | GGSLRLSCTAPR | YD | QRELVAQIN | | RTVYLQMNDLKPEDAAVY | | QVTVSS | Pacos |
| | | | | | YCNV | | | (30538) |
| 5O05 | QVQLQESGGGLVQP | SVSR | MGWYRQTPGN | SVGS | TNYADSLKGRFTISRENAE | IRYSPIHAPL | DYWGQGT | Vicugna |
| | GGSLRLSCAASG | TYV | QRELVATIT | | NTVYLQMNSLKPEDTAIYY | | QVTVSS | Pacos |
| | | | | | CKY | | | (30538) |
| 5O0W | QVQLQESGGGLVQP | TALTY | IGWFRQAPGK | RINSG | TDYADSVKGRFTISRDDAK | DTTDRYDSA | DYWGQGT | Vicugna |
| | GGSLRLSCAASE | YA | EREGVSCIS | SGAR | NTVTLQMNSLEPEDTARYY | NGRYYCTISS | QVTVSS | Pacos |
| | | | | | CAL | DTY | | (30538) |
| 5OCL | QGQLVESGGGLVQA | IIFREL | IDWYRQAPGK | SAGM | TNYADSVKGRFTISRDNAK | LPRFS | HLWGQGT | Vicugna |
| | GGSLRLSCAASG | G | QRELVASIA | | NTVYLQMNSLKPEDTAVYY | | RVTVSS | Pacos |
| | | | | | CHT | | | (30538) |
| 5OCL | QVQLQESGGGLVQA | RTWS | MGWFRQAPGK | WRGG | THYADFVKGRFTISRDNVK | NPNPSGSSV | DYWGQGT | Lama |
| | GDSLRLSCAASG | IYG | EREFVAGIT | N | NTVYLQMNSLKPEDTAVYY | YRRN | QVTVSS | Glama |
| | | | | | CAA | | | (9844) |
| 5OMN | QVQLQESGGGLVQP | TIFSR | MGWYRQAPGK | SDRS | TWYAESVEGRFTISRDNVK | RTL | GSWGQG | Vicugna |
| | GGSLRLSCAASG | NI | ERELVASIY | | NTLYLQMNSLKPEDTAMYY | | TQVTVSS | Pacos |
| | | | | | CRD | | | (30538) |
| 5TJW | QVQLQESGGGLVQA | RTST | QGWFRQPPGK | THDG | THYTDSVKGRFTISRDNAE | SLGYSGAYA | DYWGQGT | Vicugna |
| | GGSLRLTCALSE | SYA | EREFVASLR | N | NTLYLQMNSLKTEDTAVYY | SGY | QVTVSS | Pacos |
| | | | | | CAA | | | (30538) |
| 5TP3 | QVQLQESGGGLVQP | FTLDY | IGWFRQAPGK | GSSG | TYYPDSVKGRFTISRDNAK | IRSSSWGGC | DYWGKGT | Lama |
| | GGSLRLSCAASG | YY | EREAVSCIS | S | NTVYLQMNSLKPEDTAVYY | VHYGM | QVTVSS | Glama |
| | | | | | CAT | | | (9844) |
| 5TOK | QVQLQESGGGLVQP | FTLDY | IGWFRQAPGK | SSHG | TYYADSVKGRFTISRDNAK | VAVAHFRGC | DYWGKGT | Lama |
| | GGSLRLSCAASG | YY | EREGVSCIS | S | NTVYLQMNSLKPEDTAVYY | GVDGM | QVTVSS | Glama |
| | | | | | CAT | | | (9844) |
| 5U4M | QVQLVESGGGLVQP | FSRF | IGWFRQAPGK | SSSD | TSYSDSVKGRFTISRDNAK | RLGGWASFS | DYWGQGT | Vicugna |
| | GGSLRLSCVASE | TLDY | EREGLSSIS | GF | NTVYLQMNSLKPEDTAVYY | PQEY | QVTVS | Pacos |
| | | YA | | | CAA | | | (30538) |
| 5VL2 | QVQLQQSGGGLVQA | RTHT | MGWFRQAPGK | GVAA | TTYADSVRGRFTISRDDAK | RASMAVSTS | PIWGQGT | Lama |
| | GGSLRLSCAASG | PYA | EREFVGGIG | T | ATVYLQMNSLKPEDTAVYY | PRVY | QVTVSS | Glama |
| | | | | | CAT | | | (9844) |
| 5VM0 | EVQLVESGGGLVQT | RTYT | MAWFRQAPGK | GIDGT | AAYADSVRGRATISRDSAK | RASMQVLTS | PIWGRGT | Lama |
| | GDSLRLSCAASG | PYA | EREFVAGIG | | KTVYLQMNSLKPEDTAVYS | PRVY | QVTVSS | Glama |
| | | | | | CAT | | | (9844) |
| 5VM6 | QVKLQQSGGGMVQ | RALS | VGWFRQIPGK | WSSS | TWYADSVKGRFTISKDDAA | ALRRPGSDA | PYWGQGT | Lama |
| | TGDSLRLSCVGSR | STI | EREFVGGIA | D | NGVHLQMSSLKPEDTAVY | SDYTRIPDY | QVTVSS | Glama |
| | | | | | YCAS | | | (9844) |
| 5VXJ | QVQLAETGGGLAQP | FTFS | MNWYRQAPGK | DAGG | GSIADPVKGRFTISRDNAK | GIFDGNYR | TYWGQGT | Vicugna |
| | GGSLRLSCAASG | RAV | ERELVARIY | N | NTVHLQMNSLKPEDTAMY | | QVTVSS | Pacos |
| | | | | | VCNA | | | (30538) |
| 5VXL | QVQLAESGGGLVQP | GVFII | MGWYRQAPGK | SYSG | TNYADSVKGRFTISRDNVE | NLRTN | NYWGQGT | Vicugna |
| | GGSLRLSCSASG | YN | QRELVASID | SI | KRVYLEMNNLKPEDTAVYY | | QVTVSS | Pacos |
| | | | | | CNA | | | (30538) |
| 6FYT | EVOLVESGGGLVQP | SIFDI | MDWYRQAPGK | RDGS | TNYADSVKGRFTISRDNAK | SLYRDPLGV | VYWGKGA | Lama |
| | GGSLRLSCAVS | YA | QRDLVATSF | | NTLYLQMNSLKPEDTAVYL | AGGMG | LVTVSS | Glama |
| | | | | | CHV | | | (9844) |
| 6DBD | QVKLEESGGGLVQA | RIEGI | VGWYRQGPGK | RNG | NTRYDGSAEGRFTIARENA | LSSGVNP | WAWGQG | Lama |
| | EGSLRLSCVTSG | LL | QRDVVASID | | NTVYLQMNNLRPEDSNVY | | TQVTVSS | Glama |
| | | | | | VCGA | | | (9844) |
| 6DBE | QVKLEESGGGLVQA | STFSI | MGWFRQAPGK | WNGGW | TYYADSVKGRFTIYRDNYK | DDLMIDR | DYWGQGT | Lama |
| | GGSLRLSCAASG | YT | EREFVADIS | S | NTVYLQMNSLKPEDTAVYY | | QVTVSS | Glama |
| | | | | | CNA | | | (9844) |
| 6DYX | QVKLEESGGGLVQA | RTYS | MGWFRQTPGK | WSGG | THYADSVKGRFTISRDNAK | PKGHTGD | HYWGPGT | Vicugna |
| | GGSLRLSCAASG | TYA | ERELVAAIN | N | STVYLQMNSLKPEDTAVYY | | QVTVSS | Pacos |
| | | | | | CAA | | | (30538) |
| 6F5G | QVKLEESGGGLVQA | RTFS | MGWFRQVPGR | DSDA | IYYTDSVKGRFTISRDNAKN | RQYYSGRVY | DYWGQGT | Lama |
| | GGSLRLSCSPSD | AYA | EREFVATIR | S | TVYLQMNSLIPDDTAVYYC | STFREEY | QVTVSS | Glama |
| | | | | | AA | | | (9844) |
| 6H6Y | QVQLQESGGGLVQA | RTFS | SGWFRQAPGK | WSDS | TNYADSVKGRFTISRDTAK | RRLATY | DYWGQGT | Vicugna |
| | GGSLRLSCAVSG | NYY | EREFLASIR | T | NTVYLQMNSLKLEDTAVYH | | QVTVSS | Pacos |
| | | | | | CAA | | | (30538) |
| 6H70 | QVQLQESGGGLVMT | RTIDV | MAWFRQAPGK | WSGM | TYSADSVKDRFTISRDKTK | RSRFIVGVPQ | DYWGQGT | Vicugna |
| | GGSLRLSCAVSG | SV | EREFVSGMR | T | NTVYLQMNSLKPEDTAVYY | ARDLY | QVTVSS | Pacos |
| | | | | | CAA | | | (30538) |
| 6HHU | QVQLVESGGGLVQA | SIFRV | MGWYRQVTGK | SGGR | TNYADSVKGRFTISRDNIK | DLGTGGRSY | DYWGQGT | Lama |
| | GGSLRLFCAASG | NA | QRELVAAIT | | NTVYLQMNSLKPEDTAVYY | | QVTVSS | Glama |
| | | | | | CNA | | | (9844) |
| 6JRI | DVQLVESGGGLVQA | FTFD | MGWFRQAPGK | WSGS | TYYTDSVKGRFTISRDNAK | ARYRGIGSQ | IYWGQGT | Vicugna |
| | GGSLRLSCTASG | DYT | EREGVSYTG | MSGS | NTLYLQMNSLKPEDTAMYY | VRWTDF | QVTVSS | Pacos |
| | | | | T | CAA | | | (30538) |
| 6K2I | QLQLVESGGGLVQA | RTFR | MGWFRQAPGK | WSGG | TYYADSVKGRFTISRDNAK | GVWRSSGW | DYWGQGT | Vicugna |
| | GGSLRLSCVASG | SNA | EREFVAAIR | S | NTVYLQMNSLKPEDTAVYL | DTP | QVTVSS | Pacos |
| | | | | | CAA | | | (30538) |
| 6NFJ | QVQLVESGGGLVQA | RAIRS | MAWFRQAPGK | YIFN | TYYADSVKGRFTISRDNAK | GVGASVSVS | NYWGQGT | Lama |
| | GGSLRLSCAASG | YF | EREFVAAVE | | NTVFLQMNSLKPEDTAVYY | ESWY | QVTVSS | Glama |
| | | | | | CAA | | | (9844) |
| 6OBC | QVQLAETGGGLVQP | SISSF | MGWYRQAPGK | NYGR | TDYGDSLKGRFTISRDNAA | RKWERSVLE | DYWGQGT | Vicugna |
| | GGARTLSCAASE | YF | PRELVAEIS | | NTVNLQMNNLAPEDTALYY | | QVTVSS | Pacos |
| | | | | | CNA | | | (30538) |
| 6OBG | QLVETGGGLVQPGG | SIFSI | MGWYRQAPGK | SSGR | INEADSVKGRFTISRDNAK | LAGSHYYDE | EYWGQGT | Vicugna |
| | SLRLSCAASG | NA | ERELVADIS | | NTVYLQMNSLKPEDTAVYY | Y | QVTVS | Pacos |
| | | | | | CNV | | | (30538) |
| 6OBM | QVQLVETGGGGLVQ | GSISS | MGWYRQAPGK | ASGR | TNYADSVKGRFTISRDNAK | VGGTYYYDE | DYWGQGT | Vicugna |
| | AGGSLRLSCAAS | LNA | ERELVADIS | | NTVSLQMNSLKPEDTAVYY | Y | QVTVS | Pacos |
| | | | | | CNA | | | (30538) |
| 6OBO | VQLAETGGGLAQAG | SIFSI | MGWYRQAPGK | GSGR | TNYADSVKGRFTISRDNAK | VGGSYYYDE | NYWGQGT | Vicugna |
| | GSLRLSCAASG | NA | ERELVADIS | | NTVSLQMNSLKPEDTAVYY | Y | QVTVSS | Pacos |
| | | | | | CNV | | | (30538) |
| 6OC8 | EVQLVESGGGLVQA | SIFRA | VGYYRQTPGR | GGGS | THYADPVKGRFTISRDNAK | GPTLTTGDA | PYWGQGT | Lama |
| | GGSLRLSCAASA | LN | QRELIAGIS | | NRVDLQMNNLKPEDTAVY | G | QVTVSS | Glama |
| | | | | | YCNA | | | (9844) |
| 6OCA | VQLVETGGGVVQAG | RTFS | LGWFRQAPGK | WSVD | TDLANSVKGRFTISGVNAK | GLRGGTYAR | DYWGQGT | Vicugna |
| | GSLRLSCVASG | VSGR | EREFVGSIS | GDAT | NTVYLQMNSLKPEDTAVYY | TIYEY | QVTVSL | Pacos |
| | | TFSD | | YY | CAA | | | (30538) |
| | | HG | | | | | | |
| 6RBB | RGSEFEVQLVQAGG | GTFA | MGWLRQAPGK | PSGG | RIYADSVKGRFTISRDNAG | RRRFDGTSY | DSWGQGT | Lama |
| | SLRLSCAASG | TSP | GTEFVAAIS | D | NFIYLQMNSLKPEDTAVYY | YTGDY | LVTVSS | Glama |
| | | | | | CAV | | | (9844) |
| 6R7T | QVQLQESGGGLVQP | FTFST | MSWFRQAPGK | SSGG | QSYADSVKGRFTISRDNAK | AAWRVGTY | DYRGQGT | Lama |
| | GGSLRLSCAASG | YA | GLEWVASIN | GI | NTLYLQMNSLEPEDTAVYY | | QVTVSS | Glama |
| | | | | | CAA | | | (9844) |
| 6RUM | QVQLVESGGALVQP | FPVN | MRWYRQAPGK | SAGD | SSYEDSVKGRFTISRDDAR | NVGLPADLA | EYWGQGT | Lama |
| | GGSLRLSCAASG | RYS | EREWVAGMS | R | NTVYLQMNSLKPEDTAVYY | WFKRNTLNK | QVTVS | Glama |
| | | | | | CNV | PVIMGRHTW | | (9844) |
| | | | | | | ESIGRPLPGR | | |
| | | | | | | KNIILSSQPG | | |
| | | | | | | TDDRVTWVK | | |
| | | | | | | SVDEAIAACG | | |
| | | | | | | DVPEIMVIGG | | |
| | | | | | | GRVYEQFLP | | |
| | | | | | | KAQKLYLTHI | | |
| | | | | | | DAEVEGDTH | | |
| | | | | | | FPDYEPDDW | | |
| | | | | | | ESVFSEFHD | | |
| | | | | | | ADAQNSHSY | | |
| | | | | | | CFEILERRGG | | |
| | | | | | | GGGMISLIAA | | |
| | | | | | | LAVDRVIGME | | |
| | | | | | | NAMPWNF | | |
| 6U55 | QVQLQESGGGLVQP | FTLAE | VGWFRQAPGK | VGGT | THSIDSVKGRFTISRDNMK | YSSAYDR | NYWGQGT | Lama |
| | GGSLRLSCTASG | YG | ERELVALIA | | NTVYLQMNSLNVEDTAVYY | | QVTVSS | Glama |
| | | | | | CNA | | | (9844) |
| 6Z6V | QVQLVETGGGLVQA | RTFN | MAWFRQAPGT | AGGG | THYADSVKGRFVISRDNDK | DENPPGWPS | DYWGQGT | Lama |
| | GGSLRLSCAASG | NDV | EREFVALIT | | NMAYLQMNSLKSEDTAIYY | RWSSAY | QVTVSS | Glama |
| | | | | | CGA | | | (9844) |

TABLE 10B

| | | SEQ ID | SEQ ID | SEQ ID | | | SEQ ID | |
|---|---|---|---|---|---|---|---|---|
| | | NOS: | NOS: | NOS: | | | NOS: | |
| | SEQ ID NOS: | 8975- | 9991- | 11021- | SEQ ID NOS: | SEQ ID NOS: | 14072- | |
| | 7945-8974 | 9990 | 11020 | 12029 | 12030-13059 | 13060-14071 | 15101 | Source_ |
| ID | Frame1 | CDR1 | Frame2 | CDR2 | Frame3 | CDR3 | Frame4 | organism |
| 3ln9_A | EVQLVESGGGL | YTFSHRY | HRWFRQAP | QSGMR | TYYADSVKGRFTISRDNA | GTRKNVWTRQHP | DYWGQG | Camelidae |
| | VQPGGSLRLSC | | GKEREIVAV | | KNTVYLQMNSLKPEDTA | F | TQVTVSS | |
| | TASG | | IS | | MYYCAA | | | |
| 3tpk_A | EVQLVESGGG | YTFSQEF | VIWFRQAP | LRKGW | TYYADSVKGRFTISQDNA | APTATHALYF | DYWGQG | Camelidae |
| | SVQPGGSLRLS | | GKEREIVSG | | KNTVYLQMNNLKPEDTA | | TQVTVSS | |
| | CTASG | | IS | | MYYCAA | | | |
| 2x89_C | QVQLQESGGG | YTDSRYC | MAWFRQAP | SGRD | TYYADSVKGRFTFSQDN | DIPLRCRDIVAKG | RYWGQG | Camelus |
| | SVQAGGSLRLS | | GKEREWVA | | AKNTVYLQMDSLEPEDTA | GDGF | TQVTVSS | |
| | CAASG | | RIN | | TYYCAT | | | |
| 5dxw_A | QVQLVETGGG | FTFSMHA | MTWYRQAP | SHGDR | ANYTDSVRGRFTISRDNT | PRY | DSWGQG | Camelidae |
| | LVQPGGSLRLS | | GKQRELVA | | KNMVYLQMNSLKPEDTA | | TQVTVSS | |
| | CTASG | | VIT | | VYYCNV | | | |
| 5e5m_H | QVQLVESGGG | STISSVA | VGWYRQTP | TSSTT | ATYADSVKGRFTISRDNA | GL | TNWGRGT | Camelidae |
| | LAQPGGSLRLS | | GNQREWVA | | KNTIYLQMNSLKPEDTAV | | QVTVSS | |
| | CAASG | | TSS | | YYCKT | | | |
| 5imo_B | QVQLVESGGG | RTFSSYG | MGWFRQAP | WNGGS | TYYADSVKGRFTISRDNA | GRWDKYGSSFQD | DYWGQG | Camelidae |
| | LVQAGGSLRLS | | GKEREFVA | | KNTVYLQMNSLKPEDTAV | EY | TQVTVSS | |
| | CAASG | | AIR | | YYCAA | | | |
| 5jds_B | QVQLQESGGG | KMSSRRC | MAWFRQAP | TTSGS | TYLADSVKGRFTISQNNA | DSFEDPTCTLVTS | QYWGQG | Camelidae |
| | LVQPGGSLRLS | | GKERERVA | | KSTVYLQMNSLKPEDTA | SGAF | TQVTVSS | |
| | CAASG | | KLL | | MYYCAA | | | |
| 6qtl_H | QVQLVESGGG | RTGTIYS | MAWFRQAP | WSSGI | TYYMDSVKGRFTISRDKG | TRAYSVGY | DYWGQG | Camelidae |
| | LVQAGGSLRLS | | GKEREFLAT | | KNTVYLQMDSLKPEDTAV | | TQVTVS | |
| | CTASG | | VG | | YYCTA | | | |
| 6dbg_D | QVKLEESGGG | HTYSTYC | MGWFRQVP | VGGSS | TWYADSVRDRFTISQDN | HRFCNTWSLGTL | NVWGQG | Camelus |
| | SVQAGGSLRLS | | GKEREGVA | | AKNTVYLQMNSLKLEDTA | | TQVTVSS | |
| | CAASG | | RIN | | IYYCTL | | | |
| 6dyx_D | QVKLEESGGGL | RTYSTYA | MGWFRQTP | WSGGN | THYADSVKGRFTISRDNA | PKGHTGD | HYWGPGT | Vicugna |
| | VQAGGSLRLSC | | GKERELVA | | KSTVYLQMNSLKPEDTAV | | QVTVSS | |
| | AASG | | AIN | | YYCAA | | | |
| 6dbe_B | QVKLEESGGGL | STFSIYT | MGWFRQAP | WNGGS | TYYADSVKGRFTIYRDNY | DDLMIDR | DYWGQG | Lama |
| | VQAGGSLRLSC | | GKEREFVA | | KNTVYLQMNSLKPEDTAV | | TQVTVSS | |
| | AASG | | DIS | | YYCNA | | | |
| AIZ0302930.1 | QVQLVESGGG | RSSFSRYF | MGWFRQAP | WTGSS | TYYADSVKGRFTISRDNA | DFLRGSRRYGINT | DYWGQG | Camelidae |
| | SVQAGGSLSVS | | GKEREFVA | | KNTVYLQMNSLKPEDTAV | TEEY | TQVTVSS | |
| | CAASG | | AIS | | YYCAA | | | |
| CAP58559.1 | EVQLQASGGG | STFRINR | MGWYRQA | SGGS | TNYADSVKGRFTISRDNA | VTPW | PYWGQGT | Vicugna |
| | LVQAGGSLRLS | | PGKQRELV | | KGTVNLTMNSLKPEDTAV | | QVTVSS | |
| | CAASG | | ASIN | | YYCNR | | | |
| CAP58560.1 | EVQLQASGGG | STFSINV | IGWYRQAP | RSGN | TNYADSVEGRFTISRDYA | KTYLLH | MYWGQG | Vicugna |
| | LVQAGGSLRLS | | GKQRELVA | | KNTVYLQMNSLKPEDTAV | | TQVTVSS | |
| | CAASG | | GIS | | YYCHS | | | |
| CAG33966.1 | EVQLQASGGG | SIFSIDG | MGWYRQA | SGGS | TNYADSVKGRFTISRDNA | RTWR | DYWGQG | Camelidae |
| | LVQPGGSLRLS | | PGKQRELV | | KNMMFLQMRSLLPENTA | | TQVTVSS | |
| | CAASA | | AGIT | | VYYCHA | | | |
| CAG33969.1 | DVQLQASGGG | IVFRVST | MGWYRQA | SGGS | TNYADSVKGRFTIYRDNA | NTGLRTL | SFWGQGT | Camelidae |
| | LVQPGGSLRLS | | PGKQRELV | | KNTVYLQMNSLKPEDTAV | | QVTVSS | |
| | CAASG | | ATIS | | YYCNA | | | |
| CAG33970.1 | EVQLQASGGG | RTFSNAR | MAWFRQAP | WSGTT | TNYADSVKGRFTISRDNA | DGSWRGVCNNV | DYWGQG | Camelidae |
| | LVQAGGSLRLS | | GQEREFVA | | KNTVYLQMNSLKPEDTAV | Y | TQVTVSS | |
| | CAASE | | AIS | | YYCAA | | | |
| CAG33972.1 | RVQLQASGGG | RTLSSYA | MGWFRQAP | WSGTR | TSYADSVKGRFTISRDNA | RSMRSNLYTVYE | DYWGQG | Camelidae |
| | LVQAGGSLRLS | | GKDREFVA | | KNTVYLQMNSLKPEDTAV | APHDY | TQVTVSS | |
| | CAASG | | AIS | | YYCAM | | | |
| CAG33974.1 | CELQASGGGL | SIFSNHA | MGWYRQP | SGGR | INYADFVKGRFTISRDNA | WRLGY | DYWGQG | Camelidae |
| | VQPGGSLRLSC | | PGKQREFV | | KNTVYLQMNKLKPEDTAV | | TQVTVSS | |
| | AASG | | ANIF | | YLCNA | | | |
| CAG33975.1 | EVQLQASGGG | RTFSTYN | MAWFRQAP | WSGG | TYYADSVKGRFTISRDNA | SRRASYFGDPTD | RSWGQG | Camelidae |
| | LLQAGDSLRLS | | GKERELVA | | KNTVSLQMDSLKPEDTAV | F | TQVTVSS | |
| | CAASG | | AIT | | YYCTR | | | |
| AAP22639.1 | QVQLVESGGG | YTYRSTC | LGWFREAP | RGTGD | TYYGDAVRGRFTISQDNA | YSRFGYSGLC | NDWGQG | Camelus |
| | SVQAGGSLRLS | | EKEREVVA | | KKTVYLQMNSLKPEDTA | | TQVTVSS | |
| | CVASG | | AIY | | MYYCNT | | | |
| AAP22640.1 | DVQLVESGGG | YTYENRY | MAWFRQAP | RGGNN | PYYADSVKGRFTISQDKA | QAGRFSGPFTNQ | DYWGQG | Camelus |
| | SVPAGGSLRLS | | GKEREGVA | | KNIVSLLMNSLKPEDTAIY | TY | TQVTVSS | |
| | CAVSG | | AIW | | YCAA | | | |
| AAP22641.1 | QVQLVESGGG | YSVSIGC | MAWFRQAP | RGGSM | TDYTASVKGRFTISRDND | DGPEIATMIG | GSRGRGT | Camelus |
| | SVEAGGSLRLS | | GSGREGVA | | QRTVTLQMNSLKPEDTA | | QVTVSS | |
| | CVVSG | | GIS | | VYYCAR | | | |
| AAP22642.1 | DVQLVESGGG | ITYRMYC | MAWFRQPP | RIDRAG | TYYADSVKGRFTISQDNA | RDYGCYSGSAQG | AYWGRGT | Camelus |
| | SVLTGGSLRLS | | GQEREMVA | GR | KDTLYLQMNSLKPEDTAI | TEF | QVTVSS | |
| | CAAPG | | TVN | | YLCAA | | | |
| 1yc7_B | DVQLVESGGG | STYSPCT | TGWYRQAP | SPGT | IYYQDSVKGRFTISRDNA | QCGVRSIR | EYWGQGT | Camelus |
| | SVQAGGSLRLS | | GKEREWVS | | KNTVYLQMNSLQREDTG | | QVTVSS | |
| | CAVSG | | SIS | | MYYCQI | | | |
| AAP22645.1 | QVQLVESGGG | STYRRVF | TGWFRQAP | VGVGD | TYYADSVKGRFTISQDNA | FMTGTQVLRPQD | KYWGQGT | Camelus |
| | SVLAGGSLRLS | | GKEREGVA | | KNTVYLQMNSLKPEDTA | Y | QVTVSS | |
| | CAASE | | AIL | | MYYCAA | | | |
| AAL40829.1 | EVQLQASGGG | RPFSSFA | MGWFRQAP | ASGGE | TYYTGSLKGRFTISRDNA | TINGA | ARRGQGT | Lama |
| | SVQAGDSLRLS | | GKEREFVA | | KNTVYLQMDSLKPEDTG | | QVTVSS | |
| | CAASG | | AIS | | VYYCAA | | | |
| ACF49483.1 | DVQLQESGGG | YDNDNYC | MGWFRQTP | IGGGS | PVYADFVRGRFTISLDSS | IRKPQFYTCRMW | DIWGQGT | Camelus |
| | SVQAGGSLKLS | | GKEREKVA | | KDTLYLLMNAVTPEDTAM | KPRADF | QVTVSS | |
| | CAISG | | ALN | | YYCAA | | | |
| ACY01865.1 | DVQLQESGGG | DTAAYTDD | LGWFRQAT | TGSSS | TFYIDAVKGRFTFSQDND | ADFTCMPEVTAN | GHWGQG | Camelus |
| | SVQAGGSLKLA | GYC | GNEREGVA | | DKNIVYLQMNSLRPEDTA | QALNIMSGSQFAA | TQVTVSS | |
| | CTVSG | | AIN | | MYICA | G | | |
| AEA07719.1 | QVQLQESGGG | LTDSTCG | MAWYRQAP | TDGN | TYYADSVKGRFTISQDNA | WSESKRLCLPD | NYWGQG | Camelus |
| | SVQAGGSLRLS | | GKERELVST | | KNRVFLQMNSLKPEDTA | | TQVTVSS | |
| | CAASG | | | | MYYCNS | | | |
| AEX07288.1 | QVQLVESGGG | NIYRRNY | VGWFRQAP | IGSRN | TVYADSVKGRFTISQDNA | GLLRAADLGRELR | DYWGQG | Camelus |
| | SVQAGESLRLS | | GKEREGVAI | | KPTVYLQMNSLKPEDTA | SLLTSPNSV | TQVTVSS | |
| | CAGSG | | IY | | MYYCAA | | | |
| AEX07289.1 | QVQLVESGGG | HTLRSHY | MAWFRQVT | GIGSTA | TDYADSVEGRFIISRDNT | RTGFSGSWTRQT | AYWGQGT | Camelus |
| | SVQAGGSLRLS | | GKEREGVA | | ENTVYLQMADLKPEDAA | DY | QVTVFS | |
| | CASST | | YIP | | MYYCAV | | | |
| AEX07290.1 | QRQVVESGGG | FTLDEAD | MGWYRQIP | GFGQ | RYYAESVWGRFTISQDK | NRGFSRSHLTCP | GYWGQG | Camelus |
| | SVQAGETLELS | | GHECELVA | | AKNTVYLEMNSLKPEDTA | APG | TQVTVST | |
| | CTGSG | | SIG | | VYYCSA | | | |
| AEX07291.1 | QVALEECGGV | VASERCA | IYWYRQAP | RPGT | TTTIDSVKGRFSVSRDKA | LGCAQ | ATWGQGI | Camelus |
| | SVQAGGSLRLS | | GKEREWVL | | TETTFLQMDNLKPEDTG | | QVTVSS | |
| | CTAPG | | SLN | | MYTCRT | | | |
| AEX07292.1 | QMQLVESGGG | YTISTDC | MGWFRQTP | TGDGR | TYYADSVKGRFTISQDNA | RPKSGARGCADL | TYRGQGT | Camelus |
| | SVQAGGSLRLS | | GKEREGIAT | | KNTMWLEMNSLKPEDTA | WDWY | QVTVSS | |
| | CAASG | | LY | | MYYCAA | | | |
| AEX07294.1 | QAQVVETGGG | LIYMSNC | MGWFRQAP | TGSGA | TYYPDYLKGRFTITRDNA | GGGDPGNKPAIY | NYWGQG | Camelus |
| | SVKAGGSLRLS | | GKKREGVA | | KNMVFLQIDNLKVEDTAIY | SCYSGSWSREQA | TQVTVSS | |
| | CATSE | | SIY | | YCAA | ED | | |
| AEX07309.1 | QVQLVESGGG | DVYSRVC | MGWLRQAP | TGGSN | AYYADSVKGRFTISQDNA | DARRGYWRPCNS | NYWGQG | Camelus |
| | SVQAGGSLRLS | | GKERERVA | | KSTTYLQMSSLKPEDTAM | IEY | TQVTVSS | |
| | CTASG | | SIY | | YYCAT | | | |
| AEX07301.1 | QVQLQESGGG | ITHSPVC | MGWFRQAP | TAEGK | LFYLESVKGRFTFSRDTT | DPDCRATWYRGG | GAWGQG | Camelus |
| | LVQAGGSLRLS | | GKEREGVA | | KNTISLEMNGLKPDDTAM | | TQVTVSS | |
| | CVASG | | TEY | | YYCAA | | | |
| AEX07302.1 | RVHVVLSGGG | YKDDFNC | MGWFRQVP | GEWQ | PDYADFVNGRFTISQDPA | PSPGVTYGGNDQ | EVRGQGT | Camelus |
| | SVQAGGSLTLS | | GKELESVAT | G | RNTVSLQMYNLQPNDTA | PCKYL | LVIVSS | |
| | CEASG | | IA | | MYYCAT | | | |
| AEX07303.1 | QVQMVESGGG | SISSRNA | IGWFRQAP | VSGK | GVYADSVRGRFTISQNTA | DYGWKLDGRLLG | DYWGQG | Camelus |
| | KVQVGGSLTLS | | GKEREGVA | | KTTVYLQMNNLTIDDTAT | SESWGLQPSGY | TQVTVTE | |
| | CECSG | | AIY | | YVCAS | | | |
| AEX07303.1 | QVQLEEAGGG | DSGVNYC | MGWFRQAP | NR | GTTNYADSVRGRFTISKV | VHLWDGRAWYC | TYWGRGT | Camelus |
| | SVQAGGSLRLS | | GKEREGLA | | KKQLYLDMVNLKPEDTA | PERTSDF | QVTVSA | |
| | CAASG | | TIV | | MYYCAS | | | |
| AEX07313.1 | QAQLRESGGG | YTVSSHC | MGRFREAP | SRGI | ITHAGSVKGRFTISKDNS | HLARGGGYCYDL | DYWGQG | Camelus |
| | SVQPGGTLRLS | | GKEREGVA | | KNTLYLQMNSLKPEDTAL | SLSSRF | TQVTVSS | |
| | CVASG | | VID | | YYCAT | | | |
| AEX07314.1 | QEELMESGGG | FTSSRRC | MGWFRQVP | TGDSR | TNYIDSVKGRFTITHDNAK | GPRKVGSCDTSF | KYWGQGT | Camelus |
| | PVQAGESLRLS | | GQEREVVA | | DTVYLQMNSLKPEDTAM | SY | QVTVSS | |
| | CSASG | | VIF | | YFCAA | | | |
| AEX07315.1 | QVRVMESGGG | NTIDNC | VGWFRQAP | ADDGT | TVYADSVKGRFTISRDNA | NEFGVRVWSLCQ | NFWGQGT | Camelus |
| | SVQAGGSLRLS | | GREREGVA | | KNTVYLQMNNLKPEDTA | STRY | QVTVSS | |
| | CTASG | | YMH | | MYYCAV | | | |
| AEX07317.1 | QAQMVESGGG | YTYASAC | IAWFRQAP | TGRGD | TVYADSVKGRFTISQDNA | SSTTAALVWSPT | NYWGQG | Camelus |
| | SVQAGGSLRLS | | GKEREGVT | | ENTVFLQMDSLKPEDTA | CPLRGSDY | TQVTVSS | |
| | CRAST | | AIY | | MYYCAA | | | |
| AEX07318.1 | QVQLVESGGG | YVNSPNC | WAWFRQAP | TASGI | ATYDDSVKGRFTISQDNA | QRPYGN | TYYGSCT | Camelus |
| | SGQAGGSLRLT | | GKEREGVA | | KNTVLLQMDSLKPEDSA | | RVVGAY | |
| | CGASG | | TIN | | MYYCAV | | | |
| AGJ76249.1 | EVQVVESGGG | DTYSRRC | MGWFRQAP | TNGGG | TAYADPAKGRFTISQDNA | QYAASKTYGRSC | KYWGQGT | Camelus |
| | SVQAGGSLRLS | | GKEREGVA | S | KDTVYLQMQDLEPEDTAI | GTALGDY | LVTVSS | |
| | CAASG | | TIH | | YYCAA | | | |
| AFW03620.1 | DVQLVESGGG | SDITYSTD | MGWFRQAP | NGRAI | TYYADSVKGRFTISQDNA | RLRAGYCYPADY | DYWGKGT | Camelus |
| | SVQGAAGGSL | C | GKEREGVA | | KNTVYLQMNSLRPKDTAI | SM | QVTVSS | |
| | RLSCAA | | TIN | | YYCAA | | | |
| AFW03621.1 | DVQLEESGGG | YIYSTYC | MGWFRQAP | DVGGS | VYYADSVKGRFTISQDIA | LRCLSDSDPDTR | YDWGQG | Camelus |
| | SVQAGGSLRLS | | GKEREGVA | | QDTMYLQMNDLTPENTV | VHMY | TQVTVSS | |
| | CAASG | | AIN | | TYTCAA | | | |
| AFW03622.1 | DVQLEESGGG | YTYSSAC | MGWFRQG | TGGRR | TYYADSVKGRFTISQDNT | GPRRRDYGLGPC | NYWGQG | Camelus |
| | SVQTGGSLRLS | | PGKEREAV | | KDMRYLQMNNLKPEDTA | DY | TQVTVSS | |
| | CAASG | | ADVN | | TYYCAT | | | |
| AFW03623.1 | EVQLEESGGGL | YTFTNCA | AGWYRQAP | SGNR | TNYADSVKGRFTISRDNT | RT | PCWGQG | Camelus |
| | VQPGGSLTLSC | | GKECELVA | | KDIVYLQMNSLKPEDTTV | | TQVTVSS | |
| | AASG | | SIF | | YYCDA | | | |
| AFW03624.1 | EVQLEESGGG | YTFLQLL | HGWFRQAP | TDINK | TFYLESVKGRFTLSQDNA | SRPDSTCDYF | AYRGQGT | Camelus |
| | SVQAGGSLRLS | | GKEREVVA | | KNTLYLQMNSLKPEDTAI | | QVTVSS | |
| | CAASG | | RFN | | YYCAA | | | |
| AFY09731.1 | GLVGGLKARSL | SGYTESVK | MGWFRQAP | IPGGS | TYYDDHVKGRFTISQDNA | RNAGGRFRPSAA | NYWGQG | Camelus |
| | VQAGGSLRLAC | W | GQEREGVA | | QNTVYLQMNSLKPEDTA | GGY | TQVTVSS | |
| | AASA | | VIS | | MYYCAA | | | |
| AGN74801.1 | HVQLVESGGG | YTYSSNC | MGWFRQAP | AGGG | IYYADSVKGRFAISQDNA | GGSLYSGSCSRY | RYRGQGT | Camelus |
| | SVQAGGSLRLS | | GKEREGVA | W | KNMVYLQMNSLKPEDTA | WDEY | QVTVSS | |
| | CAASG | | GVY | | MYYCVA | | | |
| AGN74804.1 | HVQLVESGGG | YTYSSDS | MAWFRQAP | IGSGS | TLYADSVKGRFTISQDKA | GGNWYDGVLNVE | AYWGQGT | Camelus |
| | SVQAGGSLRLS | | GKEREGVA | | KNTVYLQMNSLKPEDTAL | RAF | QVTVSS | |
| | CTYSV | | GIY | | YYCAA | | | |
| AGN74805.1 | DVQLVESGGG | YTYSSAC | MGWFRQAP | TGSGR | TYIADSVKGRFTISQDNA | GGSYYSGSCSRY | RYWGQG | Camelus |
| | AVQAGGSLRLS | | GKEREAVA | | KNTVYLQMNSLEPEDTA | GDEW | TQVTVSS | |
| | CAASR | | GIY | | MYYCVA | | | |
| AGV76520.1 | HVQLVESGGG | YAYVSNC | LAWFRQAP | SGGGS | TYYADSVKGRFTISLDIPK | LSGVCDGGPSMT | GIGVQGT | Camelus |
| | SVQAGGSLRLS | | GKEREEVA | | SAVYLQMNSLKPDDTAM | AIQ | QVTVSS | |
| | CKASG | | AIY | | YYCAA | | | |
| AGV76521.1 | HVQLVESGGG | DGYMRNC | MGWFRQAP | TGGRS | LYYADFMKGRFTISQDNP | ETRGDCSGGYLG | NYWGQG | Camelus |
| | SVQAGGSLRLS | | GKEREGVAI | | KNTLYLQMNILEPEDTAM | PDPY | TQVTVSS | |
| | CAASG | | VA | | YYCAA | | | |
| AGV76522.1 | QVQLVESGGG | YTYSSNY | LGWFRQTP | RGGLR | TYYADSVKGRFTISRDNA | STGRLWAGYDWY | NFWGQGT | Camelus |
| | SVQAGGSLRLS | | GKEREGVA | GGGR | ENTVYLEMNGLIPEDTAM | RPEPY | QVTVSS | |
| | CTASG | | AIY | | YYCAA | | | |
| AGV76523.1 | HVQLVESGGG | HISSTCG | RGWYRQAP | SDGT | IVYADSVKGRFTISQDNV | CR | YWWGQG | Camelus |
| | SVQAGGSLKLS | | GKERELVSI | | ENTLSLQMNSLKTDDTGV | | TLVTVSS | |
| | CAVSG | | IT | | YYCAA | | | |
| AGV76524.1 | QVQLVESGGG | YTYSRYY | MGWFRQAP | SDGR | TIYADSVKGRFTISKDNAK | KIPPKTWFEY | NYWGQG | Camelus |
| | SVQAGGSLRLS | | GKEREGVA | | NTLYLQMNNLKLEDTAMY | | TLVTVSS | |
| | CVASG | | TFD | | YCAA | | | |
| AGV76525.1 | DVQLVESGGG | YTYGSNF | MGWFRQAP | GDGS | TTYAGSVKGRFTISKDNA | TRHDTRATLEDL | NYWGQG | Camelus |
| | SVQAGGSLRLS | | GKEREGVA | | HNTLYLQMNSLKPDDTAA | WY | TQVTVSS | |
| | CAASG | | YID | | YYCAS | | | |
| AGV76526.1 | QVQLVESGGG | YVYISYC | MGWFRQAP | TGGGT | TSYADSVKGRFTISQDNA | DCSIATIAPGSPLI | GFWGQG | Camelus |
| | SVQAGGSLRLS | | GKEREGVA | | KNTVYLQMNSLKPEDTA | EF | TLVTVSS | |
| | CAASG | | AIY | | MYYCAA | | | |
| AGV76527.1 | DVQLVESGGG | YTYSSNC | MGWFRQAP | TVGGS | TYYTDSVKGRFTISKDNA | GWSRADSWCYL | GYWGQG | Camelus |
| | SVQAGGSLRLS | | GKEREGVA | | KNTLYLQMNSLKPEDTTM | PDL | TQVTVSS | |
| | CAASG | | AIY | | YYCAA | | | |
| AGV76528.1 | QVQLVESGGG | YRTSNYC | MGWFRQAP | RVDT | TTYAGSVKGRFTISKDNA | SLRYYCNPKEGLF | KNWGQG | Camelus |
| | SVQAGGSLRLS | | GKEREEVA | | KNTLYLQMNSLKPEDTA | | TLVTVSS | |
| | CATSG | | TID | | MYYCAA | | | |
| AGV76530.1 | HVQLVESGGG | YTYSSYC | MGWFRQSP | SDGS | THYADSVKDRFTISKDNA | DYYSDYDESCDY | NYWGQG | Camelus |
| | SVQAGGSLRLS | | GKERQGVA | | KNTLYLQMNNLKPEDTAV | | TQVTVSS | |
| | CVASE | | VID | | YYCAL | | | |
| AGV76531.1 | DVQLVESGGG | YTYSSSNC | IGWFRQAP | TGGGI | TYYADSVKGRFAISQDNA | QRAEWGECILDD | NYWGQG | Camelus |
| | SVQAGGSLRLS | | GKEREGVA | | KNTVYLQMNSLKPEDTA | Y | TQVTVSS | |
| | CAASG | | TIY | | MYYCAT | | | |
| AGV76534.1 | EVQLVESGGG | YTYSRMC | MGWFRQTP | SAGN | TNYVDSVKGRFTISQDNA | VASVVAHRCSPS | NYWGQG | Camelus |
| | SVQAGGSLRLS | | GKEREGVA | | KNTLYLQMNSLKPEDTAI | PDY | TQVTVSS | |
| | CRASR | | AID | | YYCAA | | | |
| AGV76535.1 | QVQLVESGGG | YTYSSYC | MGWFRQAP | SDGS | TSYADSVKGRFTISQDIAK | VWGSGCSGGYP | GYWGQG | Camelus |
| | SVQAGGSLRLS | | GKEREGVA | | NTLYLQMNSLKPEDTAM | RL | TLVTVSS | |
| | CAASG | | AID | | YYCAA | | | |
| AGV76536.1 | HVQLVESGGG | FTFDDSD | MGWYRQA | SDGS | TYYADSVKGRFTISQDNA | DYGWGGYCYNLE | GYWGQG | Camelus |
| | SVQAGGSLRLS | | PGNECELV | | KNTLYLQMNSLKPEDTAL | TFGF | TLVTVSS | |
| | CTASG | | STIS | | YYCAA | | | |
| AGV76540.1 | QVQLVESGGG | YTYSSNC | MGWFRQAP | TGGGS | TYYADSVKGRFTISQDNA | PPRNREAGDCYS | NYWGQG | Camelus |
| | SVQAGGSLRLS | | GKEREGVA | | KNTVYLQMNSLKPEDTA | GSWSKPREY | TQVTVSS | |
| | CAASG | | AIY | | MYYCAV | | | |
| AGV76541.1 | QVQLVESGGG | YTYSSYC | MGWFRQAP | SDGS | TSYADPVKGRSTISKDNV | HVLCRLLKVL | GIRGQGT | Camelus |
| | SVQAGGSLRLS | | GKEREGVG | | ANTLYLQMNSLKPEDTA | | LVTVSS | |
| | CVASG | | AID | | MYYCAA | | | |
| AGV76542.1 | QVQLVESGGG | HTFSVLS | MGWFRQAP | SDGS | TRYADSVKGRFTISKDNA | DLFGLLFRLLEAE | NYRGQGT | Camelus |
| | SVQAGGSLRLS | | GKDREGVA | | KNTLYLHMNSLKPEDTAM | SY | LVTVSS | |
| | CAASG | | AID | | YYCAA | | | |
| AGV76544.1 | QVQLVESGGG | YIFSSCG | MGWYRQA | SDGT | TSYADSVKGRFTISQDNA | DPWYPAAC | NYWGQG | Camelus |
| | SVQAGGSLKLS | | PGKERELV | | KSTLYLQMNSLKTEDTAV | | TQVTVSS | |
| | CAASG | | STIS | | YYCAA | | | |
| AHW48352.1 | DVQLVESGGG | FTFDDYT | MGWFRQAP | WSGSM | TYYTDSVKGRFTISRDNA | ARYRGIGSQVRW | IYWGQGT | Camelus |
| | LVQAGGSLRLS | | GKEREGVS | SGST | KNTLYLQMNSLKPEDTA | TDF | QVTVS | |
| | CTASG | | YTG | | MYYCAA | | | |
| AHW48351.1 | DVQLVESGGG | FTFDDCT | MGWFRQAP | WSGST | TYYTDSVKGRFTISRDNA | ARYRGIGSRVRW | IYWGQGT | Camelus |
| | LVQAGGSLRLS | | GKEREGVS | SGST | KNTLYLQMNSLKPEDTA | TDF | QVTVS | |
| | CTASG | | YIG | | MYYCAA | | | |
| AID18522.1 | DVQLVESGGG | YMYSSKA | MGWFRQAP | TDGYS | PYYADSVKGRFTISQDNA | SKRGPSMGWVP | GDWGQG | Camelus |
| | SVQAGGSLRLS | | GKEREGVAI | | KNTVYLQMNSLKPEDTA | KSASYF | TQVTVS | |
| | CAGSG | | IY | | MYYCAA | | | |
| AIF28002.1 | QVQLQESGGG | GAYRNAC | MGWFRQAP | SVDT | TYYADPVKGRFTISRDNA | VARVVCPGDKLG | NYWGQG | Camelus |
| | SVQAGGSLKLS | | GKEREGVAI | | KSTVYLLMNSLKPEDTAIY | ASGY | TQVTVSS | |
| | CAASG | | IN | | YCAQ | | | |
| AIF28003.1 | QVQLQESGGG | PTYSSYF | MAWFRQAP | YDGST | TLYADSVKGRFTISQGNA | RRRGWSNTSGW | DYWGQG | Camelus |
| | SVQAGGSLRLS | | GMEREGVA | | KNTKFLLLNNLEPEDTAIY | KQPGWY | TQVTVSS | |
| | CAASG | | ASS | | YCAL | | | |
| AIF28004.1 | QVQLQESGGG | YTFSDYC | MGWFRQAP | GGKR | TYYSDSVRGRFTISRDDY | GGYTTGVCAGGF | NDWGQG | Camelus |
| | SVQAGGSLRLA | | GKEREEVA | | KNTVWLQMDSLKPEDTAI | | TQVTVSS | |
| | CAAPG | | RIS | | YYCAR | | | |
| AIF28006.1 | QVQLQESGGG | YTFSDYC | MGWFRQAP | NGGLI | TRYADSVKGRFTVSRDN | GSYTCNPDRWSQ | KYGGQGT | Camelus |
| | SVQAGGSLRLS | | GKEREGVAI | | AKNTLYLEMNSLKPEDTA | VSDY | QVTVSS | |
| | CADSG | | IS | | TYFCAK | | | |
| AIF28007.1 | QVQLQESGGG | MTFSVYN | LGWLRQAP | RDGS | TDYADSMKGRFTISRDNA | GVGVVD | CTEGQGT | Camelus |
| | SVQAGGSLRLS | | GQECELVS | | KNTMYLQMTSLKPDDTA | | QVTVSS | |
| | CESSG | | TIT | | VYYCAA | | | |
| AIF28009.1 | QVQLQESGGG | DTRRINN | MAWFRQTP | VVIGT | TDYDDSVKGRFTLSRDN | GELIRFSRWRTG | TYWGQGT | Camelus |
| | SVQTGGSLTLS | | GKEREGVA | | AKNTLYLRMNSLKPEDTA | REF | QVTVSS | |
| | CAASV | | RIN | | MYYCAA | | | |
| AIF28012.1 | QVQLQESGGG | NHDSFQT | MGWFRQAP | YVGL | TFVRASVRGRFTISQDDA | KGVADRDFTNF | DYWGQG | Camelus |
| | SVQVGGSLRLS | | GKEREGVA | | MKTMYLQMDSLKAEDTG | | TQVTVSS | |
| | CVYSG | | TIN | | VYYCAA | | | |
| AIF28013.1 | QVQLQESGGG | YTRNGCV | LNWYRLAT | TDGN | TMYRTPVKGRFTISQDSA | TGTTRWCRP | SDWGQG | Camelus |
| | SVQAGESLRLS | | GKPREWVS | | KDTAFLQMNSLKVEDTA | | TQVTVSS | |
| | CTVPG | | SID | | MYFCRT | | | |
| AIG20205.1 | DVQLVESGGG | STVTIGS | MGWFRQAP | TGRGS | TNYADSVKGRFTISQGNA | APGRPSLKYEMR | RYWGQG | Camelus |
| | SVQTGGSLTLS | | GKEREGVA | | KNTVYLQMNSLKPEDTAI | QVDF | TQVTVSS | |
| | CAASG | | LIY | | YYCAA | | | |
| AKE13334.1 | QVQLQESGGG | DTYSSAC | MGWFRQAP | TSTSM | RYYADAVKARFTISQDNA | GHTVGSSWRDPG | RYWGQG | Camelus |
| | SVQAGGSLRLS | | GKEREGVA | RT | KNTVYLQMNSLKPEDIAM | AW | TQVTVSS | |
| | CVASG | | TIC | | YYCAT | | | |
| AKE13335.1 | QVQLQESGGG | YAYDTYY | MGWFRQAP | SLVSG | KYYTDSVKGRFTIFRDDD | SRSGLRARLLRPE | EYWGQGT | Camelus |
| | SVQDGGSLRLS | | GKEREWVA | VAYY | KNTVDLQMNSLKPEDTAI | LY | QVTVSS | |
| | CAASG | | GIT | | YYCAA | | | |
| AKF02407.1 | EVQLVESGGG | YTYCSYG | MSWYRQAP | SDGS | TSYADSVKGRFTISQDNA | DQTGGSWWADC | EVWGQGT | Camelus |
| | SVQAGGSLRLS | | GKEREFVS | | KNTVYLQMNSLIPEDTAM | RYL | MVTVSS | |
| | CAASG | | AID | | YYCKT | | | |
| AKF02408.1 | QVQLVESGGG | YTCYTDD | RSWSRQAP | SDGT | TTYADSVKGRFTISRDNA | VCRDRWNVRQ | TYWGQGT | Camelus |
| | SVQAGGSLRLS | | GKEREFVSI | | KNTVYLQMNSLKPEDTG | | LVTVSS | |
| | CVASG | | IV | | MYYCKI | | | |
| ALN98710.1 | QVQLQESGGG | YIASGCGM | MAWYRQAP | RDG | IRYIGAVKGRFTISRDSAK | CRRSIDDY | TYWGQGT | Camelus |
| | SVQAGGSLKLS | GG | GKERELVST | | NTLYLQMSSLKTEDTGVY | | QVTVSS | |
| | CAASG | | IS | | YCAA | | | |
| ALN98712.1 | QVQLQESGGD | YIFSTCG | MGWYRQA | IDGT | TRYADSVKGRFTISQDRA | GGGSGSWCPG | NYWGQG | Camelus |
| | SVQAGGSLKLS | | PGKERELLS | | KHTLYLQMNSLKTEDTAV | | TQVTVSS | |
| | CAASG | | TRS | | YYCRA | | | |
| ALN98713.1 | QVQLQESGGG | YTDRNYC | MGWFRQAP | SAGN | TAYADSVKGRFTISQDNA | IAKEYNRGGYYC | DYWGQG | Camelus |
| | SVHSGGSLRLS | | GSQREGVA | | KNTLWLRMNTLKPEDTA | RSSGRV | TQVTVSS | |
| | CLAAG | | VID | | MYYCAA | | | |
| ALN98714.1 | QVQLQESGGG | YRLNLNC | MGWFRQAP | SDSY | GRYADSVRGRFAISQDN | WRGDCGIPDMLR | NYRGQGT | Camelus |
| | SVQSGGSLRLS | | GKEREGVA | | AKNTLYLQMNTLNPEDTA | TD | QVTVSS | |
| | CVASG | | TIA | | TYYCAA | | | |
| ALS19648.1 | DVQLQASGGG | FSFADSD | MSWFRQAP | RNGN | TEYSDSVKGRFTISRDNA | PKSGLLRPPISHC | KYWGQGT | Camelus |
| | SVQAGGSLRLS | | GKERELVST | | KNAVYLQMNSLKPEDTA | DSCVVLGVDGY | QVTVST | |
| | CAASG | | IR | | MYYCAA | | | |
| 5foj_A | QVQLQESGGG | DTFSGYL | AAWFRQAP | SKRHT | TSYADSVKGRFTISKDNA | ADAIGLAEYWSTP | KYWGQGT | Camelus |
| | SVQVGGSLRV | | GKGREGVA | | DNIMYLEMNSLKPEDTAI | TLSAARY | QVTVSS | |
| | ACAASG | | AIN | | YYCAA | | | |
| ASM90300.1 | DVQLQESGGG | YTYNTYRR | MGWFRQAP | SVDGR | SYYADSVKGRFTISRDNG | ADKWGGYCGGV | GYEGQGT | Camelus |
| | SVQAGGSLRLS | FC | GREREGVA | | ENAVNLQMNSLKPEDTAI | ADIAHF | QVTVSS | |
| | CAVNA | | SIN | | YYCAA | | | |
| 5u65_B | DVQLQESGGG | LTISNYC | MRWFRQAP | SAGT | TYYADSVKGRFTMSRDN | STRVWGGYCGGL | NDWGQG | Camelus |
| | SVQAGGSLRLS | | GKGREGVA | | AKNTVYLDMNSLKPEDTA | DDATN | TQVTVSS | |
| | CVVSG | | SIN | | IYYCAS | | | |
| ASM90302.1 | DVQLQESGGG | FTTTSYSS | IGWFRQAP | AKGST | LYYVDSVKGRFTISRDND | AADVYRGGYCSA | SYWGQGT | Camelus |
| | SVQAGGSLRLS | TC | GKEREAVA | | NAENTVYLQMNSLKPED | YAGRE | QVTVSS | |
| | CTASG | | TIN | | TSTYYC | | | |
| ASM90303.1 | DVQLQESGGG | YTSSKYC | VGWFRQAP | GGGGA | TSYADSVKGRFTVSQDN | GTRPSIFCYKGDR | SAWGQGT | Camelus |
| | SVQAGGSLRLS | | GKEREGVA | | AEGTVYLQMNDLKPEDT | WY | QVTVSS | |
| | CAASE | | VFI | | AIYYCAA | | | |
| 5u64_B | DVQLQESGGG | VTSTRPC | IGWFRQAP | FRGDS | TYITDSVKGRFTISRDEDS | DVNRGGFCYIED | SYWGQGT | Camelus |
| | SVQAGGSLRLS | | GKEREGVA | | DTVYLQMNSLKPEDTATY | WYF | QVTVSS | |
| | CVASG | | VVN | | YCAA | | | |
| ASM90305.1 | DVQLQESGGG | YTHRRYC | MAWFRQAP | NGGSS | TYFADSVKGRFTISRDSE | DDLRGGYCSSSS | TYWGQGT | Camelus |
| | SVQAGGSLRLS | | GKEREGVA | | ENTLYLQMNSLKAEDTGI | TLY | QVTVSS | |
| | CAASG | | SVN | | YYCAA | | | |
| ASM90306.1 | DVQLQESGGG | YTSNRNV | MGWFRQAP | TRTGS | TDYADSVKGRFTISQDNN | SYQFFSSEPSLSA | TDWGQGT | Camelus |
| | LVQPGGSLTLS | | GKEREGVA | | KNTVYLQMNSLKPEDTA | RRY | QVTVSS | |
| | CAASG | | VIN | | MYYCAA | | | |
| ASM90307.1 | DVQLQESGGG | YTHGSYC | MGWFRQAP | KSGGS | SNYPDSVKGRFTISRDNA | TETWGPYCGQAS | PYWGQGT | Camelus |
| | SVQTGGSLRLS | | GKDREVVA | | DNTVYLLMNDLKPEDTAT | PAYF | QVTVSS | |
| | CVASE | | TIN | | YYCAA | | | |
| AUS94292.1 | QVQLQESGGG | FTFGAYY | MYWFRQAP | TGDDT | TYYADSVKGRFTISQDNA | DSSG | RVRGQGT | Camelus |
| | LVQPGGSLRLS | | GKGLEWVS | | KNTLYLRMNNLKPEDTAV | | QVTVSS | |
| | CAASG | | SIY | | YYCAT | | | |
| AXU39122.1 | EVQLVESGGG | FSFSSNV | MGWFRQAP | VDSGS | TWYADSVKGRFTISLDSA | RRGVILTLSPETY | DYWGQG | Camelus |
| | SVQTGGSLRLS | | GKEREGVA | | NNTLYLQMNSLKPEDTA | | TQVTVSS | |
| | CAASG | | AIS | | MYYCAT | | | |
| BAD00517.1 | DVQLVESGGG | YTDCKYD | MSWYRQVP | KIGTD | TYYPVSVKGRFTVSRDD | GESWWGY | SDWGQG | Camelus |
| | SVQAGGSLRLS | | GKERELVS | | AKNTLNLQLNTLKIEDTA | | TQVTVSS | |
| | CAASR | | QIN | | MYYCVR | | | |
| BAD00570.1 | EVQLVESGGG | YAANRYT | VGWFRQAP | PAGTV | TEYADSVKGRFTISQDDA | GRALVGPGFKGF | TIWGQGT | Camelus |
| | SVQAGGSLRLS | | GKAHEGVA | | KNTVYLQMNNLKPEDTGI | | QVTVSS | |
| | CAAPR | | SIN | | YYCVA | | | |
| BAD00571.1 | QVQLVESGGG | YLFSRHA | MGWFRHRP | SDGT | TDYADSVKGRFTISRDNA | DNPGPPVCRGGN | GYFGQGT | Camelus |
| | SVQAGGSLRLS | | GKECELVS | | KNTVDLQMDSLKPEDTA | CRVCDTQA | QVTVSS | |
| | CKHIG | | MVL | | VYYCAA | | | |
| BAD00574.1 | EVQLVESGGG | FVFDDSD | MGWYRQA | DDGS | TYYHESVQGRFTISRDNV | GSRQFQRTSTTW | GVWAQGT | Camelus |
| | SVQAGGSLNL | | PGRECELV | | KNLMTLQMNNLKVEDTA | CL | QVTVSS | |
| | GCSASG | | ATVS | | RYYCAA | | | |
| BAD00576.1 | EVQLVESGGG | HRTGTTCS | MNWYRQAP | DFGS | TWYADSVKGRFTISEDKA | GNCNYTCHACFP | NYWGQG | Camelus |
| | TVQAGGSLRLS | | AKERELVSS | | KHVLFLQMNNLRPEDTA | PDY | TQVTVSS | |
| | CAASG | | IN | | MYYCAT | | | |
| BAD00577.1 | DVQLVESGGG | IIASNLC | MAWFRQVP | GRPGT | TLYDSSVAGRFKITRDHD | GRGWSCPSNY | DYWGQG | Camelus |
| | SVQAGGSLRLS | | EKQREAVA | T | KNTVFLQMNSLKQEDTAI | | TQVAVSS | |
| | CTVSG | | SIA | | YYCAL | | | |
| BAD00579.1 | AVQLVDSGGG | FSFDDSE | LGWYHQAP | VDGT | TYYATSVKGRFAISRDNA | DAGLNDIELKYTR | NYWGQG | Camelus |
| | SVQAGGSLTLS | | GNECELVS | | KNTVYLRMNSLKPEDSA | TDCGPLAGY | TQVTVSS | |
| | CTGSG | | FIG | | MYYCAT | | | |
| BAD00580.1 | QVKLEESGGG | FGSEYC | IGWFRQAP | SGGGN | KDYADSVKGRFTISQDNA | VPLCSRPPPMIRG | TNWGQGT | Camelus |
| | SVQAGGSLRLS | | EKEREVVA | | KNTVYLQMNSLKPEDTAI | AQHGY | QVTVSS | |
| | CAPSG | | SMN | | YYCAA | | | |
| BAD00581.1 | QVQLVESGGG | YSYSTYC | MGWFRQAP | SAT | GTTYYADSVKGRFTITQIK | DSASSVGFCSNP | DYWGKGT | Camelus |
| | SVQAGGSLRLS | | GKDREGVA | | NTVYLQMTSLTPEDTAIY | LEDM | LVTISS | |
| | CVITE | | SIN | | YCAA | | | |
| BAD00582.1 | VVQLVESGGG | YTDSVKW | VAWFRQAP | SGGFN | TYYADSVKGRFTISQDIAK | DFLSGNLLDGRY | RYWGQG | Camelus |
| | SVQAGGSLRLS | | GKEREGVA | | NTVYLQMNSLKLEDTAM | | TQVTVSS | |
| | CAASG | | CIY | | YYCAT | | | |
| BAD00583.1 | QVQLVESGGG | GPTCSHQ | MEWYRQAP | LDGR | TMYQDSVKGRFAISHDNL | TARRCGFTTTWL | ELWGQGI | Camelus |
| | SVQAGGSLRLS | | GKEREFLS | | KSTVFLQMNSLKPEDTAK | | LVNVSS | |
| | CAPSG | | GIE | | YYCKL | | | |
| BAD00584.1 | AVQLVESGGG | YTYMDYC | AGWFRQVP | SGGGK | TKYADSVKGRFTISQINA | VNSRDGADCPTG | HAWGQG | Camelus |
| | SVQAGGSLRLS | | GEAREAVA | | NNTVSLQMNSLNLEDTAV | RYDY | SQVTVSS | |
| | CTVSG | | FIN | | YYCAS | | | |
| BAD00585.1 | DVQLVESGGG | DPGINLY | ITWFRQPP | TDQNG | IYIADSVKGRFTISQDKAK | GVAASLSPHYGY | NYWGQG | Camelus |
| | SVQAGGSLRLS | | GKEREAVG | | KTVYLEMNNLKLEDTAMY | | TQVTVSS | |
| | CTVSG | | FIN | | YCAA | | | |
| BAD00588.1 | AVQLVESGGG | LSNRQLC | MAWFRQAP | SRGID | AVYADSVKGRFTISEDSA | ALAYAWSCSRAE | NYWGQG | Camelus |
| | SAQAGGSLRLA | | GKAREIVAG | | SNTVYLQMDDLKPEDTAI | AGY | TQVTVSS | |
| | CVAAE | | KK | | YYCAA | | | |
| BAD00590.1 | EVQLVESGGG | PLYSAYC | MSWFRQAP | RDGSS | TVETDSVQGRFTPSQDF | AGRRSECDSERH | PYWGQGT | Camelus |
| | SVQAGGSLRLS | | GKGREWIA | | PSNTVYLQMNSLKPEDTA | SF | QVTVSS | |
| | CTASQ | | TIN | | IYYCAA | | | |
| BAD00591.1 | AVQLVESGGG | DSDSSFC | MAWFRQGP | TYDG | STDYADSVKGRFAISQDN | VRVLAGVPCLPPP | KYWGQGT | Camelus |
| | SVKAGGSLRLS | | GKEREGVA | | HKNIYLQLHSLKPEDTAIY | YEY | QVTVSS | |
| | CATSR | | AIR | | FCAA | | | |
| BAD00592.1 | DVQLVESGGG | YTLASDL | MGWFRQAP | TVGVT | TWYADSVKGRFTISQDN | SFSGGRWSDRSR | KSWGQGT | Camelus |
| | SVQAGGNLTLS | | GEEREGVA | | NKNTVYLRMNNLKPEDT | Y | QVTVSS | |
| | CEASG | | ALN | | AMYYCSA | | | |
| BAD00595.1 | EVQLVESGGG | HTANKWC | MAWFRQVP | TYGDV | TYFRDSVKGRFIISQDNV | LRLQDLWRGSCP | NYWGQG | Camelus |
| | SVQAGGSLRLS | | GKEREGVA | | KNTVHLLMNSLKPEDTAA | ETLGY | TQVTVSS | |
| | CVASG | | AIN | | YYCAA | | | |
| BAD00596.1 | QVQLVESGGG | YTDSENYY | IGWFRQAP | TRAGT | TIYADSVKGRFTISQDKAK | TGPSCLSDSSLRP | GYRGQGT | Camelus |
| | SVQAGGSLRLS | C | GNDPEGVA | | NTSYLQMTSLTPDDMAM | WHFAGVAF | QVTVSS | |
| | CAASA | | YIT | | YYCAA | | | |
| BAD00597.1 | EVQLVESGGG | PTICITD | ISWHRQAP | ASGS | TRYAESVKGRFTVSRDS | TRVGGFCYD | GTWGQG | Camelus |
| | SVQAGGSLRLS | | GKEREFLSS | | ATNTVYLDMNSLTPDDSA | | TQVTVSS | |
| | CVASG | | IV | | MYTCQS | | | |
| BAD00598.1 | QVQLVESGGG | FASTTNC | MGWFRQAP | LGGGH | TYYADSVKGRFTISQDKA | RWAYCRSFRDSP | NYWGQG | Camelus |
| | SVQAGGSLRLS | | GKEREGVA | S | ENTVYLQMNSLKPEDTAI | | TQVTVSS | |
| | CANSR | | VIN | | YYCAA | | | |
| BAD00600.1 | AVQLVESGGG | YSYC | MGWFRQAP | SGSGT | THYADSVKGRFTISQDIA | GQVQCYTEFALR | NDRGQGT | Camelus |
| | SVQAGGSLRLS | | GKEREGVA | | KNTVYLEMNSLKPEDTAI | WLLSW | QVAVSS | |
| | CVASS | | TIN | | YYCVA | | | |
| BAD00601.1 | AVQLVESGGG | NMGRFNS | MGWIRQAP | TGGGS | AWYADSVKGRFTISQDN | DRYNCEYIRRGG | ESRGQGT | Camelus |
| | SVQTGGSLRLT | | GKGREAVA | | TKNTMNLNMNTLKPEDT | EF | QVTVSS | |
| | RALSG | | CIH | | AIYYCAA | | | |
| BAD00602.1 | QVKLEESGGG | SGYVSSYY | IGWFRRAP | SGGDS | TYYADSVKGRFTISQDNA | EGPTNIAAIYWGR | GYWGQG | Camelus |
| | SVQAGGSLRLS | | GKEREGVA | | KNTVYLQMNSLKPEDTAI | AF | TQVTVSS | |
| | CVVSA | | AIV | | YYCAA | | | |
| BAD00604.1 | AVQLVESGGG | YIFTGCG | MGWYRQA | PDGT | TTYAESVKGRFTISQDNA | LSYYSDSELGDC | NFWGQGT | Camelus |
| | SVQAGGSLKLS | | PGKEREMV | | KQTLFLQMNSLKPEDTAV | GDGE | QVTVSS | |
| | CAATT | | STIS | | YFCAV | | | |
| BAD00605.1 | QVQLVESGGG | YMYVSTS | MGWFRQAP | PFGPK | TYYADSVKGRFTISRDNA | LASGEEETVRSW | AAWGQGT | Camelus |
| | SVQAGGSLNLS | | GKEREGVA | | KHTLRMQMTNLKPEDSA | FYGRRDFSSQPD | QVTVSS | |
| | CVASG | | AIY | | MYYCGV | L | | |
| BAD00606.1 | AVQLVESGGG | DTTNNYYC | MGWFRQAP | SGGDS | SYYADSVKGRFTISQDSA | QFARATRLTCEA | DYVGQGV | Camelus |
| | SVQAGGSLSLS | | GKGLEGVA | | KNTVFLEMNSLKPEDTAI | DWYLTSAEVYVY | QVTVSS | |
| | CAASG | | AIN | | YVCAA | | | |
| BAD00607.1 | QVKLEESGGG | VPDEGNC | MGWFRQAP | TRRSI | TFYNPSVKGRFTISRDTIK | VSTAWTLCPRTP | NYWGQG | Camelus |
| | SVQAGGSLRLS | | GNEREWVA | | NTVYLQMDSLKPEDSAM | GAY | TQVTVSS | |
| | CVVSG | | QIE | | YYCAA | | | |
| BAD00601.1 | QVQLVESGGG | NIYCDYD | MNWYRQAP | SDGS | TNYADSVKGRFTISQDNA | ECYSTDYGGNVT | GYWGQG | Camelus |
| | SVQAGGSLTLS | | GKEREFVS | | LDPVYLQMNGLKPEDTA | AGPGL | TQVIVSS | |
| | CVASG | | AID | | MYICKF | | | |
| BAD00611.1 | QVKLEESGGGL | ITYPSCC | MSWYRQLP | ADGGN | RRYADSARGRFTISKGNA | DQACGSWTGPC | GAWGQG | Camelus |
| | VQAGGSLRLSC | | GKERELVAT | | KNSLYLQMNSLKPEDTAV | HANY | TQVTVSS | |
| | EASG | | IS | | YYCAA | | | |
| BAD00613.1 | DVQLVESGGG | YTYSTYY | MGWFRQAP | NGGS | TVYATSVKGRFTISQGNA | AQGLTWGSLLTP | NYWGQG | Camelus |
| | SVQAGGSLRLS | | GKKREGVA | | QKTVYLQMNSLKPEDTAI | TRY | TQVTVSS | |
| | CAASG | | AIV | | YYCAA | | | |
| BAD00614.1 | DVQLVESGGG | SRKNIAST | MGWFRQAP | RGTT | NEIYADSVKGRFTISRDN | CQDSYCYKGGFY | ALWGQGT | Camelus |
| | SVHVGGSLRLS | DC | GKDREVVA | | AQSMSLQMDRLKPEDTA | AVYPGYGC | QVNVSS | |
| | CVVSP | | TLL | | LYYCAA | | | |
| BAD00615.1 | QVQLVESGGG | FIFDASD | MGWYHQV | SKGN | TYYADSVKGRFTISRDNA | APGEVNGWRGL | DCWGQG | Camelus |
| | SVQAGGSLRLS | | PGNECELV | | KNTMYLQMNSLKPEDTA | GTTRS | TQVTVYS | |
| | CTASG | | STIS | | MYYCDT | | | |
| BAD00617.1 | AVQLVESGGG | FTVSDYS | MAWFRQVP | ADTGT | TFYADSVKGRFTISLDNA | ETAPRYYNDAVW | SFWGQGI | Camelus |
| | SVQAGGSLRLS | | GQEREVVA | | KAAVYLQMNSLNTDDTA | PSDSDF | QVTVSS | |
| | CASSG | | HIN | | VYYCAA | | | |
| BAD00618.1 | DVQLVESGGG | DTYTGNC | LGWFRQAP | IKHG | RPYYADSVKGRFTISQDS | IQPTYGIDFFGSC | DYWGQG | Camelus |
| | SVQPGGSLRLS | | GKERVMVA | | ATTTYLQMESLQPEDTA | PSESADF | TQVTVSS | |
| | CEVSG | | TIN | | MYFCAA | | | |
| BAD00619.1 | QVKLEESGGG | YTYSSNC | IGWFRQAP | TGSGS | TFYADSVKGRFTISQDNA | VGYTDYCKSPWP | RFWGQGT | Camelus |
| | SVQAGGSLRLS | | GKEREGVA | S | KNTVYLQMNSLKPEDTA | EAY | QVTVSS | |
| | CATSG | | VIY | | MYYCAA | | | |
| BAD00621.1 | EVQLVESGGG | VTYSNRC | MAWFRQGP | TRGES | PDYAASVKGRFTISQDNA | RTAEWFACPPAA | NDWGPGT | Camelus |
| | SVQAGGSLRLS | | GKEREWVA | | KNTVYLQMNSLKPEDTAT | SVDY | QVTVSS | |
| | CVLSG | | VIN | | YVCAT | | | |
| BAD00622.1 | QVQLVESGGG | YAYSSFC | WAWFRQAP | HGR | TYFGEFVKGRFTISQDNA | GCGAIQPLGAGY | NRWGRG | Camelus |
| | SVQTGGSLRLS | | GKEREGVA | | KNNVYLQMDNLKPEDTAI | | TQVTVSS | |
| | CAGSE | | AIH | | YYCAA | | | |
| BAD00623.1 | AVQLVESGGG | YTSSVYC | MGWFRQAP | SGGGS | TYIADSVKGRFTISHDNAK | HPGRNLAIQYLNA | GYWGQG | Camelus |
| | SVQAGGSLRLS | | GKEREGVA | | NTVYLQMNNLKPEDTAIY | LPGGGASYF | TQVTVSS | |
| | CAASG | | AIN | | YCAA | | | |
| BAD00625.1 | AVQLVESGGG | YTFGIYP | MAWVRQAP | SDGS | TNYYDSAKGRFTISRDNA | DGPDDIATPCPC | EVWGQGT | Camelus |
| | SVPAGGSLKLS | | GKECELVAT | | KNTVYLQMNSLKPEDTA | GAQYRFL | LVNVSS | |
| | CAASG | | LG | | RYYCAA | | | |
| BAD00627.1 | AVQLVDSGGG | YTYSDYC | MGWFRQAP | TGAGL | TYYADSVKGRFTISQDNA | GCQGLYSSCRYC | HDWGQG | Camelus |
| | SVQAGGSLRLS | | GKEREGVA | | KNTVYLLMNSLKPEDTAI | SVY | TQVTVSS | |
| | CAASG | | SIN | | YYCAA | | | |
| BAD00628.1 | QVQLVESGGG | FAYTSYC | LGWFRQVP | PSSGA | TTYPDSTKGRFTISQNDA | GHCKFWYPRLV | ASWGQGT | Camelus |
| | SVQAGGSLRLS | | GKEREGVA | | WYTVYLQMNDLKPEDTA | | QVTVSS | |
| | CSASG | | RIV | | TYYCSA | | | |
| BAD00629.1 | QVQLVESGGG | YTYSNFC | MAWFRQAP | TGGN | SYYADSVKGRFTISQDNA | DNGPGTCIGRPE | RYWGRGT | Camelus |
| | SVQAGGSLRLS | | GQAREGVA | | KNTVYLQMNSLKPEDTAI | YLF | QVTVSS | |
| | CAASG | | YIY | | YYCAA | | | |
| BAD00631.1 | QVQLVESGGG | YTRCRYD | MNWYRQAP | SDGT | TTYATSVKGRFTISQDNA | VILCGDSDMA | NYWGQGI | Camelus |
| | SVQAGGSLTLS | | GKNREFVS | | KNTATLQMASLKPEDTA | | QATVSS | |
| | CAASR | | GID | | MYYCNR | | | |
| BAD00632.1 | QVQLVESGGG | YTDRYCT | MGWYRQA | KNSR | PWYKDSVKGRFTISLNKD | WADRCSPGYGL | HLWGHGT | Camelus |
| | SAQAGGSLRLA | | PGKQRELV | | NSSLELQMDNLKPEDTAT | | QVTVSS | |
| | CGASG | | ANVI | | YYCNI | | | |
| BAD00633.1 | DVQLVESGGG | FSFSRYC | TGWFRQAS | GAGMP | TYYADSVKGRFTISRDNS | RSTFCTGGTWRD | NYWGQG | Camelus |
| | SVQPGGSLRLS | | GKEREGVA | | KDMLYLQINLLKTEDAAIY | RDEV | TQVTVSS | |
| | CTASG | | AIN | | YCAA | | | |
| BAD00634.1 | QVQLVESGGG | YTFSSNI | MAWFRQAP | SDGD | TFYGNSVKGRAAISRDDA | RVFVPPINCPADF | GYWGQG | Camelus |
| | SVQASGSLRLS | | GKECELVA | | DNTVHLQMNSLKPEDTA | | TQVTVSS | |
| | CDASE | | RMT | | VYYCVA | | | |
| BAD00635.1 | DVQLVESGGG | YIDRRDC | IGWFRQAP | TVGGN | TYYSDSVKGRFTISLDTT | NFLVSTSSVLSPL | GDWGQG | Camelus |
| | SVQAGGSLRLS | | GQEREGVA | | NNTVYLEMTSLKPEDTAM | NF | TQVTVSS | |
| | CDASG | | AIY | | YYCAA | | | |
| BAD00636.1 | EVQLVESGGG | TTDIFAY | MAWFRQAP | TGLGF | TYYADSVKDRFTISRDNA | TSGALGGSKLRR | NFWGQGT | Camelus |
| | SVQAGGSLRLS | | GKEREGVA | | KNTVYLQMNSLKPDDTA | EGY | QVTVSS | |
| | CAASK | | AAY | | MYYCAA | | | |
| BAD00640.1 | EVQLVESGGG | STSRLFC | MAWYRHVP | LVSYTT | TYYHDSVKGRFALSRDN | LPGQCGRQDDEY | RYWGQG | Camelus |
| | SVHVGGSLRLS | | GKERERVA | TIK | AGPTWYLQMDDLKLDDT | | TQVNVSS | |
| | CAASG | | AMY | | AMYSCAA | | | |
| BAD00641.1 | AVQLVESGGG | DIDKMSC | MGWFRQAP | IGSSHI | TYYHDSIKGRFTISQSQD | AAGGGGCGASM | DYWGQG | Camelus |
| | SVQAGGSLRLS | | GKGRKPVA | | NAKRMVYLQMNSLKPED | SPGGF | TQVTVSS | |
| | CVLSG | | TTY | | TGLYYC | | | |
| BAD00642.1 | QVQLVESGGG | YVYTEYTC | MGWFRQAP | RDSGS | SYYDDFVAGRFTISQDNA | GIRVPDGSCLSEN | THWGQGT | Camelus |
| | SVEAGGSLRLS | | EKKREGVA | | KATVYLQMDTVKPEDTAS | TIW | QVTVSS | |
| | CTVSG | | CIR | | YVCAI | | | |
| BAD00643.1 | EVOLVESGGG | FTFTASKW | LGWYRLTP | SDGN | RWSSDSVKARFTMSSDN | VLFDVKQGVVPN | DYWGQG | Camelus |
| | SVQAGGSLKLS | A | ENKCEFIST | | AKNTVYLEMNSLKPEDSA | APSPNCTGYF | TQVTVSS | |
| | CSVTG | | IH | | MYFCAA | | | |
| BAD00645.1 | QVQLVESGGG | HRFIHNC | MGWFRQAP | TGDGF | TYYADSVKGRFTISQGNA | APYHCDSASWRV | IFWGEGT | Camelus |
| | SVQAGGSLRLS | | GKEREGVA | | KNTVYLEMNSLKPEDTA | KEG | QVTVSS | |
| | CASSE | | SIY | | MYYCAS | | | |
| BAD00646.1 | QVQLVESGGG | ITYSNNC | MAWFRQAP | TSDGS | THYADSMKGRFTMSQDN | RKGGAGPCGGN | GYWGQG | Camelus |
| | SVQAGGSLRLS | | GKEREGVA | | AKNTVYLQMNSLKPEDTA | WAANLGRF | TQVTVSS | |
| | CAASV | | AIY | | LYYCVA | | | |
| BAD00647.1 | EVQLVESGGG | LTDSFYC | MAWFRQAP | TSGGT | TYYADSVKGRFTISQDNA | VPRFLRCDYYFS | DYWGQG | Camelus |
| | SVQAGGSLRLS | | GKEREGVGI | | KKTNVLLMNNLKPEDTAI | RGTF | TQVTVSS | |
| | CAASG | | IN | | YYCAA | | | |
| BAD00648.1 | AVQLVESGGG | LTYSLCT | MDWYRQAP | RGSLK | TYYADSVKGRLNISKDNA | GRAYLDDPTWLK | DYWGQG | Camelus |
| | SVQAGGSLRLS | | GKEREGLA | | KSTFYLQLRSLKPEDTAM | GTGF | TQVTVSS | |
| | CAASG | | AIY | | YYCAG | | | |
| BAD00649.1 | QVQLVESGGG | DTGTGGH | MGWFREAP | TGGGV | TWYADSVRGRFAASQDN | ADSCVTFARVQG | PYWGKGT | Camelus |
| | SVQAGGSLRLS | YC | GKEREEIVA | | AKNTVSLQMNSLESEDTA | GTLDYSSI | LVTVSS | |
| | CAVSG | | LY | | MYYCAA | | | |
| BAD00652.1 | QVKLEESGGG | DTGSLHY | LAWFRQHS | ISNGVG | TYYADSIKGRFTISRDPG | AGRTKYLRLVAS | HYWGQG | Camelus |
| | SVQVGGSLTLS | | GKEREGVA | GR | KEITLYLKMDNLNSEDTA | GLSTLSTQAY | TPVTVSS | |
| | CVVSG | | SIN | | SYYCA | | | |
| BAD00655.1 | QVQLVESGGG | ATYNLYNG | LGWYRQAP | VHDGS | TYYADSVKGRFTISQDNA | DRSCLPLGNRL | CLLGPGD | Camelus |
| | SVQAGGSLRLS | KC | GKEREGVA | | QNTVYLQMNGLKPEDTAI | | QVTVSS | |
| | CAASG | | HIG | | YYCAQ | | | |
| BAD00656.1 | QVQLVESGGG | SIDNGYC | IAWFRQAP | GGGGS | YAYDGSMTGRVTISQDTT | RRRVAICSPGVG | GDWGQGI | Camelus |
| | SVQAGGSLNLA | | GEEREVVA | SE | KTTVFLEMSSLRPDDTAI | WTAY | QVNVSS | |
| | CKASQ | | VIE | | YYCAA | | | |
| BAD00657.1 | QVKLEESGGG | YTSSRYC | MGWFRQAP | SGGYN | TDYADSVKGRFTISQDNT | NFSPNYALSCRTT | YYWGQGT | Camelus |
| | SGQAGGSLRL | | GKEREWVA | | KNTVYLQMNSLKPEDTAI | LGD | QVTVSS | |
| | SCASSG | | RIN | | YYCAA | | | |
| BAD00662.1 | QVQLVESGGA | FDFADSD | MGWYRQV | SAGI | PLYADSVKGRFTISRDNA | DRIRLGFPRQTPA | SDWGQG | Camelus |
| | SAQAGESLRLS | | PGNECELA | | KNAVYLQMNSLKVGDTG | CTDLRALF | TQVTVSS | |
| | CTASG | | SSIS | | MYYCAA | | | |
| BAD00663.1 | AVQLVESGGG | DTARNYY | WAWFRQVP | TGAGS | RNYADSVKDRFTISRDDT | DARTGGDLRRAS | QSWGRG | Camelus |
| | SVQAGGSLRLS | | GQEREWVA | SIQ | KNAIYLQMSTLKPEDTAM | TY | TQVTVSS | |
| | CAASG | | WID | | YYCGA | | | |
| BAD00665.1 | DVQLVESGGG | ATSRSSC | MGWFRQAP | STAIS | TNYADSVKGRFIISRDNA | GRYGIVNFARCEE | VHWGQG | Camelus |
| | SVEAGGSLRLS | | GKEREAIAG | | ENEVYLQMNNLKPEDTAI | SDF | TQVTVSS | |
| | CKVSG | | IS | | YYCAS | | | |
| BAD00666.1 | QVQLVESGGG | SGISYIM | MTWYRQAP | SDGS | TYYAVSVRGRFTISKGRA | SLECCPVSPSDG | DYWGQG | Camelus |
| | LVQAGGSLTLS | CC | GKERELIST | | KNTLSLQMNNLNVNDTA | TPRLTKVEF | TQVTVSS | |
| | CAPSG | | IS | | VYYCAQ | | | |
| BAD00667.1 | QVQLVESGGG | ANYCSYD | IGWYRQAP | LDGR | TDYADSVKGRFTVSQDN | ELLCYGDYSFMT | YRWGQG | Camelus |
| | SVQAGGPLALS | | GKEREFVS | | SKRTSYLQMNSLKPEDTA | GQ | TQVTVSS | |
| | CEASG | | RID | | MYFCKA | | | |
| BAD00668.1 | EVQLVESGGGL | FTHSSYC | IGWFRQTP | SGGGS | TYYADSVKGRFTISQDKA | DIASYDTSPCTVIA | FDYGQGT | Camelus |
| | VQAGGSLTLSC | | GQERENVA | | KNTVSLQMNNLQPEDTA | GLEY | QVTVSS | |
| | AASG | | NIN | | TYYCAA | | | |
| BAD00669.1 | QVQLVESGGG | FDGIHC | MAWYRLAP | SGGR | TAYADSVKGRFTISRDNT | NPFRCDWSYVPP | LYWGQGT | Camelus |
| | SVQAGGSLRLS | | GTEREGVA | | KNTVFLQMNSLKPEDTAI | ATAF | QVTVSS | |
| | CSASG | | TFS | | YLCAA | | | |
| BAD00672.1 | AVQLVESGGG | YTYTSSS | MGWFRQAP | SGGGN | AFYADSVKGRFIISQDHV | DPKLVCGSVAPTI | SYWGQGT | Camelus |
| | SVQAGGSLKLS | GC | GKEREGVA | | QNTVYLQMNNLKPEDTAI | LLFRLLSMG | QVTVSS | |
| | CAVSG | | TID | | YYCAA | | | |
| BAD00675.1 | DVQLVESGGD | DTYNKR | LTWFRQSP | Y | DRTNYADSVKGRFTISQA | RRSSNKPYFDLR | NYWGQG | Camelus |
| | SVQAGGSLRLS | | GREREGIAT | | KSTVYLQMNSLNPEDTA | SY | TQVTVSS | |
| | CAASG | | IF | | MYYCAL | | | |
| BAD00676.1 | QVQLVESGGG | DTDNLYS | LSWFRQAP | RGGGN | PKYSDSVKGRFTISQDVA | RKDAHVWTSLLY | NYWGQG | Camelus |
| | SVQAGGSLRLS | | GKEREGVA | | SNMVYLQMNSLKPEDTAI | PNQY | TQVTVSS | |
| | CTASG | | GIR | | YYCAA | | | |
| BAD00677.1 | QVQLVESGGG | TTNLNMC | MGWFRQSP | AGGPS | TYYDDSVRGRFVISRDIT | KRLSCADNWVRV | DYWGRGT | Camelus |
| | SVQPGGSLSLS | | GKEREGVA | TYDAT | KKTVYLQMNSLKAEDTAV | SAP | QVTVSS | |
| | CEVSG | | SFY | | YYCAA | | | |
| BAD00679.1 | EVQLVESGGG | YRYSFCS | MAWYRQAP | DDGS | TYYADSVKGRFTISQDNA | VVGGYCYHPYTM | DYWGKGT | Camelus |
| | SVQAGGSLRLS | | GKERELVS | | KKTVYLQMNNLKPEDTA | | LVTISS | |
| | CVVSG | | STI | | MYYCKI | | | |
| BAD00680.1 | QVKLEESGETS | LTFDNVH | MGWYRKTP | SSGE | IWYHMSVQGRFTISRVNE | EGVDWFKGGCS | GRWSQG | Camelus |
| | VMAGGSLRLS | | GSECEMVA | | KRTAYLQMDNLAPSDTA | | TRVTVSS | |
| | CSVSD | | EIK | | MYYCSA | | | |
| BAD00681.1 | QVQLVESVGD | FTETTCV | MGWYRQA | SDGG | TWYKDSVKGRFTISQEYV | IVAPPHERCGELE | RYWGQG | Camelus |
| | SVQAGGSLRLS | | PGKERELV | | KKTVYLEMNSLEPEDTAM | RAY | TQVIVSS | |
| | CVASG | | SSIL | | YYCMI | | | |
| BAD00682.1 | QVKLEESGGG | YTYDSNI | MGWFRQAP | TAGGG | ASYATSVQGRFIISQDDN | DYDPDYSDYNAG | GYWGQG | Camelus |
| | SVQAGGSLRLS | | GKEREGVA | | KNAVYLQMNNLKPEDTA | KELLPSDF | TQVTVSS | |
| | CAASG | | WIY | | MYYCAA | | | |
| BAD00683.1 | AVQLVDSGGG | YTDSSGC | MGWFRPAP | GELGS | TYYADSVKGRFTISQDTA | RPRAGPYDDCLG | YVWGQGT | Camelus |
| | SAQTGGSLRLS | | GKEREGVAI | | KNTVVLLMNSLKPEDTAI | SYWGGRFE | QVTVSS | |
| | CVASG | | IK | | YYCGA | | | |
| BAD00685.1 | DVQLVESGGG | FTFSKCS | MGWYRQA | IDGT | TYVVDSVKGRFVISQDNA | RDAPLGDCYVSS | DYWGKGT | Camelus |
| | SVQAGGSLRLS | | PGKERELV | | KDTVYLQMNSLKPEDTA | WSSQTM | LVTISS | |
| | CAASG | | SSIQ | | MYYCYT | | | |
| BAD00686.1 | EVQLVESGGG | FTNSDRC | MGWFRQTP | FDGDI | IYYADSVKDRFLISRDNAK | RQFSVCAGALMP | DDWGQG | Camelus |
| | SVQAGGSLRLS | | GKEREGVA | | STLYLQMNSLKPEDTAIY | SAY | TQVTVSS | |
| | CAASG | | SIN | | YCAA | | | |
| BAD00687.1 | QVQLVESGGG | FTSNSDYC | IAWFRQAP | SGGGA | THYAESVTGRFTISRDNA | DYGGWCRVKER | TYWGQGT | Camelus |
| | SVQAGGSLRLS | | GKEREGVA | | KNVVILHMNNLKPEDTAIY | GY | QVTVSS | |
| | CAASG | | AIS | | YCAA | | | |
| BAD00688.1 | QVKLEESGGG | HLDSEYC | MAWFRQAP | RHGDS | TTYGDSVKGRFTISQDNA | QYFCLKATMTSN | ILWGQGT | Camelus |
| | SVQAGGSLRLS | | GKEREGVA | | KNAVYLQMDSLKVEDTAI | RY | QVTVSS | |
| | CETSE | | SLK | | YYCAL | | | |
| BAD00690.1 | DVQLVESGGG | YIYSSSC | MGWIRQVP | SGGGT | THYHPSVKDRFTISQDNA | SNRSRFCLSYYS | DVWGQG | Camelus |
| | SVQAGGSLRLS | | GKEREGVG | | KNTIYLQMNTLKPEDTAIY | DYVDMGVI | TQVTVSS | |
| | CVASV | | GIY | | YCAA | | | |
| BAD00691.1 | QVQLVESGGG | WISSRYC | LIWFRQAPG | TGGGV | TYYSDSVKGRFTISQGNT | FLVGFDDLVCRP | NFWGQGL | Camelus |
| | SVQAGGSLRLS | | KEQEGVASI | | KNTWYLQMNNLKPEDTSI | DVSIYPS | QVTVSS | |
| | CTVSG | | N | | YTCGT | | | |
| BAD00693.1 | QVQLVESGGG | FTFDSSD | MGWYRQA | SGRA | IYYGDSVKGRFTISRDNN | YVDVDGSKDITCG | YRSQQGT | Camelus |
| | SVQTGGSLRLS | | PGNECELV | | KNTVYLQMNSLKPEDTA | RRSDFG | QVTVSS | |
| | CTTSG | | ATIT | | MYYCAA | | | |
| BAD00695.1 | QVQLVESGGG | TFDINAC | VSWFRQPP | YSDDG | TYYADSVKDRFTISQDNE | AVGSLRGQCLGR | NFWGQGT | Camelus |
| | SVQAGGSLRLA | | GKDREGVA | | NTKKTVYLQMDNLKPEDT | RLPY | QVTVSS | |
| | CEVSG | | GIN | | AIYYC | | | |
| BAQ56198.1 | QVQLQESGGD | SGYTESV | MGWFRQAP | IPGGS | TYYDDDVKGRFTISQDNA | RNAGGRFRAWA | NYWGQG | Camelus |
| | SVQAGGSLRLA | KW | GQEREGVA | | KNTVYPQMNSLKPEDTA | GGGD | TQVTVSS | |
| | CAASA | | VIS | | MYYCAA | | | |
| BAQ56201.1 | QVQLQESGGG | YIYSNYC | MAWFRQAP | GRDDR | TIYAASLQGRFTISRDNAK | DTSPLCGPATWR | DYWGQG | Camelus |
| | SVQAGGSLRLS | | GKEREGVA | | NTVYLQMNNLKPEDTAIY | NINGY | TQVTVSS | |
| | CAASG | | HIK | | YCAA | | | |
| BAR73350.1 | QVQLQESGGG | YTYRSYC | MAWFRQAP | SRGGT | TYYADSLKGRFTISQDNA | RYSATCPRTLSGF | TYWGQGT | Camelus |
| | SVQAGGSLRLS | | GEEREGVA | | KNTVYLEMNSLKPEDTAI | | QVTVSS | |
| | CAASG | | AIN | | YYCAA | | | |
| CCF72105.1 | QVQLRESGPG | DSITANY | WSWIRQSP | YSGS | TVYSKSHGSRASITRDTS | SGSLDTNDALVA | AYWGQGT | Camelus |
| | LVKPSQTISLT | YD | GKELEWIAA | | KNQISLQLNSVTPEDTAV | WAF | QVTVSS | |
| | CTVSG | | VA | | YYCAR | | | |
| CCF72114.1 | QVQPVESGGG | YGSCKYD | TNWYRQAP | DDRS | TFYADSVKGRFTISEDND | NIVMYGPCPRR | EFWGQGT | Camelus |
| | SVQAGESLRLS | | GKEREFVS | | NVKNFVYLQMNSLKPED | | QVTVSS | |
| | CAASL | | RIK | | TAMYVC | | | |
| 022284 | GGGSVQAGGS | YSNCPLT | WSWYRQFP | PDGN | TKYTYSVKGRFTMSRGS | ALQPGGYCGYGA | CLWGQGT | Camelus |
| | LRLSCAASG | | GTEREFVS | | TEYTVFLQMDNLKPEDTA | | QVTVSS | |
| | | | SMD | | MYYCKT | | | |
| 022285 | GGGSVQAGGS | DSFSRFA | MSWFRQAP | SNGR | TTEADSVQGRFTISRDNS | VSLMDRISQH | GCRGQGT | Camelus |
| | LRLSCTASG | | GKECELVS | | RNTVYLQMNSLKPEDTA | | QVTVSL | |
| | | | SIQ | | VYYCGA | | | |
| 022286 | GGGSVQTGGS | FSFSTSC | MAWFRQAS | SGGGR | TYVAESVKGRFAISQDNA | VPAHLGPGAILDL | KYWGQGT | Camelus |
| | LRLSCAVSG | | GKQREGVA | TYYN | KTTVYLDMNNLTPEDTAT | KKY | QVTVSS | |
| | | | AIN | | YYCAA | | | |
| 022287 | GGGSVQGGGS | YTYGSFC | MGWFREGP | NGGTN | TYYADSVKGRFTISQDST | ELSGGSCELPLLF | DYWGQG | Camelus |
| | LRLSCAISG | | GKEREGIAT | | LKTMYLLMNNLKPEDTGT | | TQVTVSS | |
| | | | IL | | YYCAA | | | |
| 022288 | GGGSVQAGGS | DTGT | MGWFRQAP | PDGM | TFIDEPVKGRFTISRDNA | DWKYWTCGAQT | GQWGQG | Camelus |
| | LTLSCVYTN | | GKECERVA | | QKTLSLRMNSLRPEDTAV | GGYF | AQVTVSS | |
| | | | HIT | | YYCAA | | | |
| 022291 | GGGSAQAGGS | IPLNGYY | IAWFRQAP | GGRDV | TYYADSVTGRFTISRDSP | GSRFSSPVGSTS | NYWGQGI | Camelus |
| | LRLSCAAHG | | GKGREGVA | | KNTVYLQMNSLKPEDTAI | RLESSDY | QVTASS | |
| | | | TIN | | YFCAA | | | |
| 022292 | GGGSVQPGGS | ATYSDYS | IGWIRQAPG | TGATS | KFYVDFVKGRFTISQDNA | ADPSIYYSILGIEY | KYWGQGT | Camelus |
| | LTLSCTVSG | | KDREVVAA | | KNTVYLQMSFLKPEDTAI | | QVTVSS | |
| | | | AN | | YYCAA | | | |
| 022293 | GGGSVQAGGS | FPYSTFC | LGWFRQAP | SAGGN | TYYADAVKGRFTISQGNA | DSPCYMPTMPAP | DDFGQGT | Camelus |
| | LRLSCTGSG | | GKEREGVA | | KNTVFLQMDNLKPEDTAI | PIRDSFGW | QVTVSS | |
| | | | GIN | | YYCAA | | | |
| 022294 | GGGSVQAGGS | YTITDYC | MAWFRQAP | VVRSD | TDYADSVKGRFTISQGNT | TSSFYWYCTTAP | NVWGQG | Camelus |
| | LRLSCAASD | | GKERELVA | TRL | KNTVNLQMNSLTPEDTAI | Y | TQVTVSS | |
| | | | AIQ | | YSCAA | | | |
| 022295 | GGGSVQVGGS | HTDSSTC | IGWFRQAP | FGDGG | TNYRDSVKGRFTISQLNA | TEIEWYGCNLRTT | TRWGQGT | Camelus |
| | LRLSCVAST | | GKEREGVA | | QNTVYLQMNSLKPEDSA | F | QVTVSS | |
| | | | SIY | | MYYCAI | | | |
| 022296 | GGGSVQVGGS | GTPDRVPK | LAWFRQAP | TKDGK | TFYADSVKGRFTIFLDND | NQLAGGWYLDPN | AIWGQGT | Camelus |
| | LKLSCKISG | S | EKEREGIAV | | KTTFSLQLDRLNPEDTAD | YWLSVGAY | HVTVSS | |
| | | | LS | | YYCAA | | | |
| 022297 | GGGSVQAGGS | SPSSTYC | LGWFRQAP | TDGSV | IYAADSVKGRFTISQDTAK | RLTEMGACDARW | NYWGRGT | Camelus |
| | LRLSCNVSG | | GKEREGVT | | KTVYLQMNNLQPEDTAT | ATLATRTFAY | QVTVSS | |
| | | | AIN | | YYCAA | | | |
| 022299 | GGGSVQAGGS | KYMPCTYD | MTWYRQAP | IDGK | TTYADSVKGRFTISQDSA | DSYPCHLL | DVWGQG | Camelus |
| | LRLSCASSS | | GKEREFVS | | KNTVYLQMNSLKPEDTA | | TQVTVSS | |
| | | | SIN | | MYYCKI | | | |
| 038135 | QVQLVESGGG | AAFRSSR | MAWFRQAP | TDAS | TNYPDSVKGRFTISRDNA | LPTSF | TCGGQGT | Camelus |
| | SVQAGGSLRLS | | GKECEWVS | | KNTVYLQMSSLKPEDTAV | | QVTVSS | |
| | CAYSG | | TIG | | YYCEK | | | |
| 038136 | QVQLVESGGG | VNHCINV | MSWYRQVP | NDGT | TRYAGDVKGRFTISQDNA | EPLSGGRRYGSC | NYWGQG | Camelus |
| | SVQAGGSLRLS | | GKGREFVS | | KNTVYLEMNSLKPEDTA | GL | TQVTVSS | |
| | CAASG | | SIA | | MYYCLR | | | |
| 1kxt_F | QVQLVASGGG | YTFSSYP | MGWYRQA | SDGS | ANYADSVKGRFTISRDNA | GPGSGKLVVAGR | NYWGQG | Camelus |
| | SVQAGGSLRLS | | PGKECELS | | ANTAYLQMDSLKPEDTAV | TCYGP | TQVTVSS | |
| | CAASG | | ARIF | | YYCAA | | | |
| 038138 | QVQLVESGGG | YTYSSCT | MTWYRQAP | GDGR | ISYADSVKGRFTISRDNA | SPYSGSRCPIPT | NFWGQGT | Camelus |
| | SVQAGGSLRLS | | GKEREFVS | | GNLVYLQMNSLKPEDTA | | QVTVSS | |
| | CAASG | | VID | | MYYCKA | | | |
| 1kxv_D | QVQLVESGGG | NTLCTYD | MSWYRRAP | NDGT | TTYVDSVAGRFTISQGNA | SLRYGLPGCPI | IPWGQGT | Camelus |
| | TVPAGGSLRLS | | GKGRDFVS | | KNTAYLQMDSLKPDDTA | | QVTVSS | |
| | CAASG | | GID | | MYYCKP | | | |
| 1kxq_H | QVQLVESGGG | YTDT | VGWFRQAP | RRTGY | TYSADSVKGRFTLSQDN | GNSVRLASWEGY | FYWGQGT | Camelus |
| | SVQAGGSLSLS | | GKEREGVA | | NKNTVYLQMNSLKPEDT | | QVTVSS | |
| | CAAST | | AIY | | GIYYCAT | | | |
| 038141 | QVQLVESGGG | YTYTRRC | MAWFRQAP | IDGGR | TDYADSAKGRFTISQDRA | DGGRLDPYCSIKA | RYWGQG | Camelus |
| | SVQTGGSLRLS | | GKEREGVA | | KNTVYLQMNSLKPEDTA | YAY | TQVTVSS | |
| | CAASG | | LIY | | MYYCAG | | | |
| 1g6v_K | QVQLVESGGG | YTVSTYC | MGWFRQAP | GGS | TYYGDSVKGRFTISQDNA | STVASTGWCSRL | HYRGQGT | Camelus |
| | SVQAGGSLRLS | | GKEREGVA | | KNTVYLQMNSLKPEDTAI | RPYDY | QVTVSS | |
| | CAASG | | TIL | | YYCAG | | | |
| 038143 | QVQLVESGGG | ITFGSHC | MTWFRQAP | SGSVT | TKYADSVKGRFTISRDNA | NYYCTFVPGNY | AFWGQGT | Camelus |
| | LVQAGGSLRLS | | GKEREGVA | | KNTVYLEMNSLKPEDTAI | | QVTVSS | |
| | CVTSG | | SIT | | YYCAG | | | |
| 038144 | DVQLVESGGG | YTASTYC | MAWFRQAP | RSSS | TYYDVSVKGRFTVSQDN | IEEYCGGTYWLR | KHWGQG | Camelus |
| | SVQAGGSLRLS | | GKEREGVA | | AKNTVYLQMNSLKPEDTA | PNKY | TQVTVSS | |
| | CAASG | | TIN | | IYYCAA | | | |
| 4ppt_A | QVQLVESGGG | YPHPYLH | MGWFRQAP | SGGGG | TLYADSVKGRFTISRDKG | GGYELRDRTY | GHWGQG | Lama |
| | LVQAGGSLRLS | | GKEREGVA | | KNTVYLQMDSLKPEDTAT | | TQVTVSS | |
| | CAASG | | AMD | | YYCAA | | | |
| 1mvf_B | QVQLVESGGG | FTYSRKY | MGWFRQAP | IDNGN | TIYADSVQGRFTISQDNA | SSRWMDYSALTA | NSWGQG | Camelus |
| | SVQAGGSLRLS | | GKEREGVA | | KNTVYLQMNSLKPEDTA | KAY | TQVTVSS | |
| | CAASG | | AIF | | MYYCAA | | | |
| 3eba_A | QVQLVESGGG | YTYI | SGWFRQAP | SSDGT | TYYADSVKGRFTISQDNA | TEVAGWPLDIGIY | DYWGQG | Camelus |
| | SVQAGGSLRLS | | GKGLEWVA | | KNTVYLQMNSLKPEDTA | | TQVTVSS | |
| | CSASG | | AIR | | MYYCAA | | | |
| 1ri8_A | DVQLVESGGG | YKDRNYC | MGWFRRAP | SSGR | TAYADSVKGRFTISRDVA | GWSSLGSCGTNR | NYWGQG | Camelus |
| | SVQAGGSLRLS | | GKEREGVA | | LDTAYLQMNSLKPEDTA | NRY | TQVTVSS | |
| | CAVSG | | VID | | MYYCAA | | | |
| 1zmy_A | QVQLVESGGG | YTIGPYC | MGWFRQAP | MGGGI | TYYADSVKGRFTISRDNA | DSTIYASYYECGH | DSWGQG | Camelus |
| | SVQAGGSLRLS | | GGEREAVA | | KNTVTLQMNSLKPEDTA | GLSTGGYGY | TQVTVSS | |
| | CTASG | | AIN | | MYYCAA | | | |
| 1zv5_A | DVQLVESGGG | VTYKNYC | IGWFRQAP | SDGGI | TYYADSVKGRFTISQDNA | GYRNYGQCAT | RYWGQG | Camelus |
| | SVQAGESLRLS | | GKDREGVV | | KNTVYLQMNSLKPEDTAS | | TQVTVSS | |
| | CAASG | | FIN | | YYCAA | | | |
| 1zvy_A | DVQLVESGGG | STDSIEY | MTWFRQAP | THTGN | TYYTDSVKGRFTISQDKA | TRKYVPVRFALD | DYWGQG | Camelus |
| | SVQAGGSLRLS | | GKAREGVA | | KNMAYLRMDSVKSEDTAI | QSSY | TQVTVSS | |
| | CAASG | | ALY | | YTCGA | | | |
| 2p4a_D | QVQLVESGGG | YPWTYIY | MGWFRQAP | SGGGG | TLYADSVKGRFTISRDKG | GGDALVATRY | GRWGQG | Camelus |
| | LVQAGGSLRLS | | GKEREGVA | | KNTVYLQMDSLKPEDTAT | | TQVTVSS | |
| | CAASG | | AMD | | YYCAA | | | |
| 3eak_B | QVQLVESGGG | GSEYSYST | LGWFRQAP | SMGGL | TYYADSVKGRFTISRDNS | VRGYFMRLPSSH | RYWGQG | Camelus |
| | LVQPGGSLRLS | FS | GQGLEAVA | | KNTLYLQMNSLRAEDTAV | NF | TLVTVSS | |
| | CAASG | | AIA | | YYCAA | | | |
| 3g9a_B | DVQLQESGGG | DTFSSYS | MAWFRQAP | RDGT | TTYAGSVKGRFTISRDDA | DSGTQLGYVGAV | DYWGKGT | Camelus |
| | SVQAGGSLRLS | | GKECELVS | | KNTVYLQMVNLKSEDTA | GLSCLDYVM | QVTVSS | |
| | CAASG | | NIL | | RYYCAA | | | |
| 3jbc_7 | QVQLQESGGG | YTQSSAC | MGWFRQAP | RFFGT | AYYADSVKGRFTISQDKA | GQGCLTTIQALGG | NAWGQG | Camelus |
| | SVQTGGSLRLS | | GKEREGVA | | KNTVYLQMNSLKPEDTAI | AYGY | TQVTVSS | |
| | CAASE | | GIS | | YYCAA | | | |
| 3jbd_7 | QVQLQESGGG | FTFSHGY | MAWFRQAP | TSGVS | AYYADSVLGRFTISQDNA | TSISCSSGY | MFWGQG | Camelus |
| | SAQTGGSLRLS | | EKEREWVA | | KSTLYLQMNNLKPEDTA | | TQVTVSS | |
| | CAASG | | CVR | | MYYCAA | | | |
| 3jbe_7 | QVQLQESGGG | YQYSLLC | MAWFRQVL | TYNGA | MRYADTVKGRFTVSQDK | GRWRFGDICHEG | NYWGQG | Camelus |
| | SVQAGGSLRLS | | GEGREGVA | | DKNTVYLQMNSLKPEDT | SGY | TQVTVSS | |
| | CAASG | | FIT | | AIYYCAA | | | |
| 3jbg_7 | QVQLQESGGG | YIPSAN | CMRWFRQA | RGGY | TWHADSVKGRFTISGDN | SNTCPGASADF | RSWGQG | Camelus |
| | SVQAGGSLRLS | | PKEREWVA | | AKTAAYLQMNSLKPEDTA | | TQVTVSS | |
| | CAASE | | SVL | | IYYCAY | | | |
| 3ogo_H | QVQLVESGGAL | FPVNRYS | MRWYRQAP | SAGDR | SSYEDSVKGRFTISRDDA | NVGF | EYWGQGT | Camelus |
| | VQPGGSLRLSC | | GKEREWVA | | RNTVYLQMNSLKPEDTA | | QVTVSS | |
| | AASG | | GMS | | VYYCNV | | | |
| 3qsk_B | QVQLVESGGG | YHHPYIY | MGWFRQAP | SGGGG | TLYADSVKGRFTISRDKG | GGHHLRDHTY | GQWGQG | Camelus |
| | LVQAGGSLRLS | | GKEREGVA | | KNTVYLQMDSLKPEDTAT | | TQVTVSS | |
| | CAASG | | AMD | | YYCAA | | | |
| 5c2u_B | QVQLQESGGG | DYYA | IGWFRQAP | SRDGS | TYYPDAVKGRFTISRDNA | DRRQRWGPYYYL | VYWGQGT | Camelus |
| | LVQPGGSLRLS | | GKEREGVA | | KNTVYLQMNSLKPEDTAV | SALEY | QVTVSS | |
| | CVVSG | | AIS | | YYCAA | | | |
| 5e7f_C | QVQLVESGGG | FTFDDSD | MGWYHQA | SDGS | TYYADSVKGRFTISRDNA | ATTTVASPPVRHV | GYWGQG | Camelus |
| | SVQAGGSLRLS | | PGNECELV | | KNTVYLQMNSLKPEDTA | CN | TQVTVSS | |
| | CTASG | | SAIF | | MYYCAA | | | |
| 5fuc_V | DVQFVESGGG | YIYSTYC | MGWFRQAP | TNSGR | TYYADSVKGRFTISQDNA | RPSIRCASFSATE | KDWGQG | Camelus |
| | SVHAGGSLRLN | | GKEREGVA | | KNTVYLQMNSLKPEDTAI | Y | TQVTVSS | |
| | CATSG | | HIY | | YYCAA | | | |
| 5ku2_7 | QVQLQESGGG | YAVSLYS | MGWFRQAP | SSGVD | TTYADSVKGRFTISRDNA | GFGLSLSRYTY | AHWGQG | Camelus |
| | SVQTGGSLTLS | | GKELEGVA | | KDTMYLQMNSPKPEDTAI | | TQVTVSS | |
| | CAASG | | GIS | | YRCAA | | | |
| 5m7q_B | QVQLQESGGG | NTGR | MGWFRQAP | RLGGY | TSYAGPVKGRFTISQDNA | DSRPIYSGTW | RYWGQG | Camelus |
| | SVQAGGSLRLS | | GKEREKVAI | | KNTVYLLMNSLKPEDTAI | | TQVTVSS | |
| | CAVSE | | IT | | YYCAA | | | |
| 5my6_B | QVQLQESGGG | YIFNSCG | MGWYRQS | GDGD | TWHKESVKGRFTISQDN | CYNLE | TYWGQGT | Camelus |
| | SVQAGGSLKLT | | PGRERELV | | VKKTLYLQMNSLKPEDTA | | QVTVSS | |
| | CAASG | | SRIS | | VYFCAV | | | |
| BAD00201.1 | DVQLVESGGG | ATYCTYD | MSWYRQAP | SDGS | ARYADSVKGRFTISRDNA | VFKSWCSDGLGT | NYWGQG | Camelus |
| | SVQAGGSLRLS | | EKDYEFVSV | | KNTVYLQMNSLKPEDTG | TLP | TQVTVSS | |
| | CAASG | | ID | | MYYCKT | | | |
| BAD00202.1 | EVQLVESGGG | YDFSANN | LYWLRQAP | SDGT | TDYADSVKGRFTISRDNA | DISAPPGIGGTCA | DYYGQGT | Camelus |
| | SVQPGGSLRLS | | GKECEFVS | | ENKVYLQMNSLKPEDTA | FLG | QVTVSS | |
| | CAARG | | SID | | VYYCAA | | | |
| BAD00203.1 | EVQLVESGGG | YTFSSYS | MAWFRQAP | SDGS | TNYADSVKGRFTISRDNA | DGGYYSCGVG | FERGQGT | Camelus |
| | SVQAGGSLRLS | | GKECELVS | | KNTVYLQMNALKPEDTAV | | QVTVSS | |
| | CVASG | | SID | | YYCAA | | | |
| BAD00205.1 | EVQLVESGGG | YTSSMNA | MGWFRQAP | RGGK | AYYADSVKGRFTVSRDN | TDESPLRRRFSLL | DWWGRG | Camelus |
| | SVQAGGSLKLS | | GKEREGVA | | VKNTVDLQMKGLKAEDT | DRRRY | TQVFVSS | |
| | CGLSG | | AVS | | ATYYCAA | | | |
| BAD00206.1 | AVQLVESGGG | FPFNGYC | MAWFRQAP | EGRL | TYYNDAVKGRFTISRDNT | KSYMCGSTLWRR | NDWGQG | Camelus |
| | VVHAGGSLTLS | | GQEREGVA | | KSTAELQMNNLRPEDTA | IDQY | TQVTVSS | |
| | CATSG | | AIS | | RYFCAT | | | |
| BAD00207.1 | AVQLVESGGG | YTYSSAR | IGWFRQAP | TDGVT | TYYADAVKGRFTISRDNA | RTSPYSGGWFRV | NGWGQG | Camelus |
| | SVQTGGSLRLS | | GKEREGVA | | KNTVYLQMNSLKPEDTA | SQY | TQVTVSS | |
| | CVASG | | AIL | | MYYCAI | | | |
| BAD00209.1 | QVQLVESGGG | YTISGIC | MGWFRQAP | PGGTG | TYYADSVKGRFTISRDNA | ACATSCVGGYCR | RYWGQG | Camelus |
| | SVQAGGSLRLS | | GKEREGVA | | KNTVDLQMNGLNPEDTAI | GNGY | TQVTVSS | |
| | CAASG | | GIS | | YYCAA | | | |
| BAD00210.1 | EVQLVESGGG | YTISKNR | MAWFRQAP | SGGSS | TLYADSVKGRFTISRDNA | VLRGIGYPNLRSD | KYWGQGT | Camelus |
| | SVQTGGSLRLS | | GKEREGVA | | RNAVYLQMNSLKPEDTG | AY | QVTVSS | |
| | CAASG | | TIS | | TYYCAG | | | |
| BAD00211.1 | DVQLVESGGG | ITYCTGD | RSWYRQAP | NDGT | ASYADSVKGRFTISQDIV | DFVDGTWCAIKF | THWGQGT | Camelus |
| | SEQAGGSLRLS | | GKEREFVS | | KKSVYLRMNSLKPEDTA | GR | QVTVSS | |
| | CAAPN | | SIN | | MYYCKT | | | |
| BAD00212.1 | EVQLVESGGG | FTFANSV | MVWYRQG | TDGT | TYYSTSVKGRFTISRDNA | DFQASTVGRCDG | AKWGQGT | Camelus |
| | SVQAGDSLRLS | | SGNECKLV | | KNTVYLQMNNLKAEDTA | YGY | QVTVSS | |
| | CTTSG | | SSIS | | MYYCAA | | | |
| BAD00213.1 | AVQLVESGGG | NYC | MAWFRQAP | RDGRT | TAYADSVKGRFTISRGNE | YVGGSYSCGTLE | KYWGQGT | Camelus |
| | SVQAGGFLRLS | | GKEREGVA | | KNTVYLLMNNLKAEDTAT | NDGY | QVTVSS | |
| | CVASV | | AIN | | YYCAA | | | |
| BAD00214.1 | EVQLVESGGG | NTYSTNL | MGWFRQAP | CGRGT | TFYADSVKGRFTISQDNA | GSVRGIWSGTSQ | KYWGQGT | Camelus |
| | SVQAGGSLRLS | | GKEREGVA | | KKMVYLQMELLRPEDTGI | Y | QVTVSS | |
| | CAAPG | | AIC | | YYCAS | | | |
| BAD00216.1 | EVQLVESGGD | STYSGNC | MAWFRQAP | TDDDT | TYYADSVKGRFTISQDTA | RDAWRRIGSWRD | DYWGQG | Camelus |
| | SVQAGGSLRLS | | GKEREGVA | | KNTLYLQMNSLKPEDTA | VAIY | TQVTVSS | |
| | CVASS | | VVY | | MYYCAT | | | |
| BAD00217.1 | EVQLVESGGG | YTSGRNY | MAWFRQAP | STGGS | ARYADSVKGRFTISQDLS | GKPYGDMLDARG | KYWGHGT | Camelus |
| | SVQAGGSLRLS | | GKEREGVG | | NDTMYLQMNNLKPEDTG | Y | QVTVSS | |
| | CAASG | | RIY | | MYYCAA | | | |
| BAD00235.1 | QVQLQESGGG | YTTCNSD | MNWYRQAP | SDGS | TSYADSVKGRFTISCDNA | VVLCGHSDYE | TYWGQGT | Camelus |
| | SVQAGGSLRLS | | GKEREFVS | | KNTVHLQMNSLKPEDTA | | QVTVSS | |
| | CAATR | | GID | | MYYCKR | | | |
| BAD00242.1 | QVQLQESGGG | YTHCNYG | MSWHRQAP | SDGS | TSYADSVKGRFTISQDNA | ILLCGDSDYA | THWGQGT | Camelus |
| | SVQAGGSLRLS | | GKEREFVS | | KNTVYLQMDSLKPEDTA | | QVTVSS | |
| | CAASG | | AID | | MYYCKR | | | |
| BAD00221.1 | EVQLVESGGG | YTFCSYD | MNWYRQVP | SDGL | TSYADSVKGRFTISLDIAK | DPGPFSDSRCAY | NAWGQG | Camelus |
| | SVQAGGSLRLS | | GKEREFVS | | NTVYLQMNSLKPEGTAM | | TQVTVSS | |
| | CAASA | | AID | | YYCKT | | | |
| BAD00222.1 | EVQLVESGGG | YTYCSYD | MSWYRQAP | SDGS | TSYADSVKGRFTISQDNA | DTVPFFLMGPYG | NYWGQG | Camelus |
| | SVQAGGSLRLS | | GKEREFVS | | KNTVYLQMNSLKPEDTA | GSCHG | TQVTVSS | |
| | CAASG | | AID | | MYYCKT | | | |
| BAD00224.1 | EVQLVESGGGL | FTFSWAY | MSWFRQAP | SDGSN | TYYKDSLKGRFTISRDNA | GQLLG | RSRGQGT | Camelus |
| | VQPGESLRLSC | | GKELEWVS | | KNTLYLQMNSLKSEDTAL | | QVTVSS | |
| | AASG | | GIN | | YYCNI | | | |
| BAD00226.1 | QVQLVQSGGG | FTFDDSD | MGWFHQAP | SNGN | TYYVDSVQGRFTISRDNA | RNDLVSSNTPPS | NYWGQG | Camelus |
| | SVQAGGSLRLS | | GNECELVS | | KNTVYLQMNSLKPEDTA | NCHPYGY | TRVTVSS | |
| | CTASG | | SIS | | MFYCAT | | | |
| BAD00228.1 | EVQLVESGGG | FTYSTNC | MGRFRQVP | TRRCS | TYYADSVKGRFTISQDNA | TEQVFFCSWLRG | GWWGQG | Camelus |
| | SVQAGGSLRLS | | GKEREGVA | | KNTVYLQMNSLKPEDTA | VDF | TQVTVSS | |
| | CAGSG | | TIY | | MYYCAA | | | |
| BAD00236.1 | QVQLVQSGGG | HTYCSYD | MSWYRQAP | SDGS | TTYADSVKGRFTISQDNA | GVRGGYCPID | NYWGQG | Camelus |
| | SVQAGGSLRLS | | GKEREFVS | | KNTVYLQMNSLKPEDTA | | TQVTVSS | |
| | CAASG | | AID | | MYYCKS | | | |
| BAD00237.1 | QVQLQESGGG | YTTSSCS | MAWYRQAP | SDGS | TYYADSVKGRFTISQDNA | KFKPPLFSCKLGY | NSWGQG | Camelus |
| | SVQAGGSLRLS | | GKERELVST | | KNTVDLQMNSLKPEDTA | | TQVTVSS | |
| | CAASG | | | | MYYCNM | | | |
| BAD00240.1 | QVQLVQSGGG | YTYSSYC | MGWFRQAP | SGGGR | TYYTDSVKGRFTISQDNA | EWYGGSWYPISQ | GYWGQG | Camelus |
| | SVQAGGSLRLS | | GKEREGVA | | KNTVYLQMNSLKPEDTAI | IPYGDCDF | TQVTASS | |
| | CAASG | | TIN | | YYCAA | | | |
| BAD00243.1 | EVQLEESGGG | YTYCRYD | MSWYRQAP | SDGT | TMYADSVKGRFTISRDNA | EIMCGDSDYA | SQWGQG | Camelus |
| | SVQAGGSLSLS | | GKEREFVS | | KNTATLQMNSLKPEDTA | | TQVTVSS | |
| | CAASG | | SID | | MYYCKR | | | |
| BAD00244.1 | QVQLVQSGGG | YTACSYD | MSWYRQAP | TDGS | TSYADSVKGRFTISQDNA | DGVYCSGGYRW | DYWGQG | Camelus |
| | SVQAGGSLRLS | | GKEREFVS | | KNTVYLQMNSLKPEDTA | S | TQVTVSS | |
| | CAASG | | AIE | | MYYCKT | | | |
| BAD00245.1 | QVQLVQSGGG | FTARSYF | MGWFRLAP | SRAGA | TYYADSVKGRFTISQDNA | AVGWLSGAWTW | QYWGQG | Camelus |
| | SVQAGGSLRLA | | GKEREGVA | | ENTVYLQMNSLKPEDTAI | GSKY | TQVTVSS | |
| | CAASG | | AIN | | YYCAA | | | |
| BAD00247.1 | QVQLQESGGG | YTYRSNY | MGWFRQAP | SGGGS | TRIADSVKGRFTISQDNA | GRYWYSM | DYWGKGT | Camelus |
| | SVQAGGSLRLS | | GKERERVA | | KNTVYLQMNSLKPEDTA | | LVTISS | |
| | CVASG | | HIN | | MYYCGA | | | |
| BAD00252.1 | QVQLQESGGG | GTYSTNY | MGWFRQAP | TGGRS | TDYADAVKGRFTISQDNA | GFRGLLSRVLSPS | RYWGQG | Camelus |
| | SVQAGGSLRLS | | GKEREGVA | | KNTVYLQMNSLKPEDTA | NY | TQVTVSS | |
| | CVASR | | AIY | | MYYCAA | | | |
| BAD00254.1 | QVQLQESGGG | YIYCGYSI | MTWYRQAP | LDGT | TTYADSVKGRFTISQDNA | C | NYWGQG | Camelus |
| | SCRLEGLLKLS | | GKEREFVA | | KNTVYLQMNSLKPEDTA | | TQVTVSS | |
| | CTASM | | GTD | | MYYCKR | | | |
| BAD00256.1 | QVQLVQSEGG | KTYCNYD | MNWYRQAP | SDGS | TSYADSVKGRFTISQDNA | ILLCGDSDYE | SHWGPGT | Camelus |
| | SVQAGGSLRLS | | GKEREFVS | | KNTVYLQMNSLKPEDTA | | QVTVSS | |
| | CAASG | | SID | | MYYCKR | | | |
| BAD00257.1 | EVQLVESGGG | YRVCNSD | MSWYRQAP | SDSS | TSYADSVKGRFTISQDNA | VVLCGDSDYE | TTWGQGT | Camelus |
| | SVQAGGSLRLS | | GKEREFVS | | KNTVYLQMNSLKPEDTA | | QVTVSS | |
| | CAASK | | GID | | MYYCKI | | | |
| BAD00261.1 | QVKLEESGGG | YKFSICG | MGWYRQA | RDGR | TTYADSVKGRFTISQDNA | ADQFDCSWVGGY | NAWGQG | Camelus |
| | SVQAGGSLKLS | | PGKQRELV | | KSTLYLQMNGLKPEDTAV | | TQVTVSS | |
| | CAASG | | STIT | | YFCAA | | | |
| BAD00262.1 | QVQLVESGGG | YTYNTYC | VGWFRQAP | SGSVF | TYYADSVKGRFTISEDKA | EGTQLFNRAKCG | KTWGQGT | Camelus |
| | SVQAGGSLKLS | | GMEREGVA | | KKTWYLQMNSLKPEDTAI | TWSAY | QVTVSS | |
| | CVGSG | | GMY | | YYCAI | | | |
| BAD00263.1 | QVQLVESGGG | YTASYIY | MAWFRQAP | SEDGR | TSYADSVKGRFIISQGVA | VSDYDTGPPLLPL | KYWGQGT | Camelus |
| | SVQAGGSLRLS | | GKEREGVA | | KNTVDLQMNNLKPEDSA | ETYGY | QVTVSS | |
| | CTASG | | GIY | | TYYCAV | | | |
| BAD00264.1 | QVQLVESGGG | YIPFRDYD | LNWFRRAP | RDGG | TAYAASAKGRFTMSRDN | DLSLRC | NGGGQGT | Camelus |
| | SVQAGGSLRLS | | GKECEFVS | | AKSMVYLRMNNLKPEDT | | QVTVSS | |
| | CTASG | | RIV | | AVYYCAE | | | |
| BAD00266.1 | QVQLVESGGG | DTSGTYC | MAWFRQVP | SGDSG | ILYADPVKGRFTISQDNAK | TPTAYGGSFCLM | NYWGQG | Camelus |
| | SVQAGGSLRLS | | GKEREGVA | GGT | NSVYLEMNSLKPEDAAIY | LSGGY | TQVTVSS | |
| | CVASG | | SIN | | YCAA | | | |
| BAD00267.1 | DCQLVESGGG | YDTYC | MGWFRQAP | TGGGS | AYHDSVKGRFTISQDNV | ARPHAYCNGVTA | LSWGQGT | Camelus |
| | SVQAGGSLRLS | | GKERDWVA | | KNTVYLQMNSLKPEDTAI | PLNF | QVTVSS | |
| | CAASG | | VIN | | YYCVA | | | |
| BAD00270.1 | EVQLVESGGG | FTFDDSE | MAWYQQAP | SSGG | TSYAESVKGRFIISRDNAK | QDCSGAYCYTMD | PCGGQGT | Camelus |
| | SVQAGGSLRLS | | GNECELVA | | NTVYLQMNSLEPEDTAM | PN | QVTVSS | |
| | CTASG | | TIS | | YYCAT | | | |
| BAD00271.1 | DVQLVESGGG | VTYSSNY | MGWFRQAP | TDGRS | TYYADSVKGRFAVSLDNA | VRWRYGSTCRSA | NYWDWG | Camelus |
| | SVQAGGSLRLS | | GKEREGVA | | KNTVSLQINSLKVEDTAM | LFASGVN | QGTQVTV | |
| | CAASG | | VIC | | YYCAA | | | |
| BAD00272.1 | QVKLEESGGG | YSTNRCI | MAWYRQAP | TGT | TYYAASVKGRFTISQNNA | PSCGEVADGVLG | SFWGQGT | Camelus |
| | SVQAGGSLRLS | | GKERELVS | | KNTVYLQMNALKPEDTA | WGDF | QVTVSS | |
| | CTASG | | SIN | | MYYCNM | | | |
| BAD00274.1 | DVQLVESGGG | STYCDID | MGWYRQA | PDGS | ATYADSVKGRFTMSQDG | EKSRGPRCYGY | NTWGQGT | Camelus |
| | SVQAGGSLRLS | | PGKEREFV | | AKNTVNLQMNSLKPEDT | | QVTVSS | |
| | CTISG | | SLMD | | AKYYCKI | | | |
| BAD00280.1 | EVQLVESGGG | LPSDVVA | MAWYRRAP | SGNQ | PYYADFVAGRFIISQDNT | MFFFGPDRKAK | ITFGQGTQ | Camelus |
| | SGQAGDSLRLS | | GKERELVA | | KKTLFLQMNSLKPEDTAT | | VTVSS | |
| | CATSG | | SLP | | YYCNS | | | |
| BAD00283.1 | DVQLVESGGG | LTISTAC | MGWFRQAP | KGIGG | TWYAASVKGRFTISQDNA | DLSRLCQYRMTA | AHRGQGT | Camelus |
| | SVQAGGSLRLS | | GKEREGVA | | KNTVYLQMNSLEPEDTGI | PRDF | QVTVTS | |
| | CAASG | | SIN | | YYCAA | | | |
| BAD00285.1 | EVQLVESGGG | YTYSSYC | MGWFRQAP | SFGGS | TYYADSVKGRFTISQDNA | STRWLLPHEQSN | NYWGQG | Camelus |
| | SVQAGGSLRLS | | GKEREGVA | | KKTVYLQMNSLKPEDTAI | SLQY | TQVTVSS | |
| | CAASG | | HIN | | YYCAA | | | |
| BAD00286.1 | QVQLVESGGG | FTFDDSD | MGWYHQA | SDGR | TYYEDSVKGRFTISRDNA | PSLAGGSWEGYD | GYWGQG | Camelus |
| | SVQAGGSLRLS | | PGNECELV | | KNTVFLQMNNLKPEDTA | C | TQVTVSS | |
| | CTASG | | GSIS | | MYYCAS | | | |
| BAD00289.1 | AVQLVDSGGG | YTSCPGN | MAWYRQAP | GDGS | AKYAESVKGRFTISQDND | ERPSSYRLGCIHS | AYWGQGT | Camelus |
| | SVQAGGSLRLS | | GKEREFVSI | | KNAVYLQMNSLKPEDTAT | Y | RVTASS | |
| | CAASG | | IE | | YYCKV | | | |
| BAD00291.1 | DVQLVESGGG | NVDSNNC | MGWFRQAP | LGGPG | TDYADSVKGRFTISQDNT | AKGFLFTGSCYS | AYWGQGT | Camelus |
| | SVQAGGSLRLS | | GKEREVVA | | ENTLYLQMNSLKPEDTG | GSWSDSL | QVTVSS | |
| | CVVSG | | TIY | | MYYCAA | | | |
| BAD00293.1 | QVQLVESGGG | DTIRSTSM | MGWFRQAP | TDGS | TIYADSVKDRFAISQDNV | GNCYAGSWCCV | NFWGQGT | Camelus |
| | SVESGESLTLS | | GKECELVAT | | KNSVYLQMNSLKPEDTA | APRRRF | QVTVSS | |
| | CTADA | | IR | | VYYCAV | | | |
| BAD00294.1 | EVQLVVSGGG | YTGSWCS | MGWFRQAP | SDGD | TYYDDSVKGRFTISQDYA | ETGGRPLRVGYCI | MHWGQG | Camelus |
| | SVQAGGSLRLS | | GREREMIAV | | KSMVFLPMNSLEPEDTA | DGT | TQVTVSS | |
| | CAASG | | IT | | MYYCNM | | | |
| BAD00295.1 | QVQLVESGGG | YTGTTCR | MAWYRQAP | SHGD | TYYAESVKGRFTISQDND | IACPAGVSCTRCY | EFWHWG | Camelus |
| | SVQAGGSLKLS | | GKERVLVS | | KKTVYFQMNSLKPEDTA | SEP | QGTQVTV | |
| | CAASG | | SIT | | MYLCNT | | | |
| BAD00296.1 | QVQLVESGGE | DSRSGDIL | SGWFRQAP | RRGGT | TYYSNSVKGRFTISQDKS | TTSGLWARRRRS | NYWGQG | Camelus |
| | SVEAGGSLRLS | A | GKEREGVA | | KNTVYLTMNSLAPEDTAI | DW | TQVTVSS | |
| | CVSSG | | AIG | | YYCAA | | | |
| BAD00297.1 | EVQLVESGGG | YTSSYPC | MAWFRQAP | ADGG | TAYADSVKGRFTISRDGD | KPRRGCVFYRSA | DWWGQG | Camelus |
| | SVEAGGSLRLS | | GKEREGVA | | KNTIDLLINSLKLEDTAIYY | Y | TPVTVSS | |
| | CAAAT | | VAD | | CAA | | | |
| BAD00298.1 | DVQLVESGGG | SPYC | MGWFRQAP | RRGIS | TFDSDHLKGRFTITQDNA | DNRVLYCGEGML | NYRGQGT | Camelus |
| | SVEAGGSLRLS | | GGSQEGIA | | ENTVYLIMNSLIPEDTAIY | GPTEY | QVTVSS | |
| | CVASF | | AVN | | YCAA | | | |
| BAD00300.1 | QVKLEESGGG | YTGILTC | MGWFRQVP | AR | SYYTDSVEGRFTISRDNY | KRTGVCGLGGNF | TDWGRGT | Camelus |
| | SVEAGGSLTLS | | GKEREEVA | | KNMVQLHMTRLTPEDTG | | QVTVSS | |
| | CTAPE | | YIG | | MYYCGA | | | |
| BAD00302.1 | QVQLVESGGG | PINT | LAWFRQAP | PRYFR | TFYADSVKGRFTISEGDD | SAGRLDEPLGAR | LTWGSGL | Camelus |
| | SVQAGGSLTLS | | GAEREGVA | RDN | EDTVYLQMNNLQPEDTA | NY | QVTVSS | |
| | CKNTV | | AIY | | MYYCTA | | | |
| BAD00303.1 | AVQLVESGGG | FTYTAYT | MGWFRQTP | SGGAS | TYYTDSVKGRFTISRGNM | SPSLSASAWSTGI | EFWGPG | Camelus |
| | SVQAGGSLSLS | | EKERERVA | | KNTLSLQMLSLIPEDPAV | SAF | MQVTVFS | |
| | CAAFG | | AIS | | YYCAA | | | |
| BAD00304.1 | AVQLVESGGG | YTNDNYC | VAWFRQAP | KVGGG | TWYADSVKGRFTISQDH | GSTWETCT | DYWGQG | Camelus |
| | SVQSGQSLRLS | | GKEQEAVA | K | ARSAKYLQMDSLKPEDT | | TQVTVSS | |
| | CTVFR | | GLT | | AMYYCAA | | | |
| BAD00305.1 | QVKLEESGGG | NTLTVNC | MGWFRQTP | ADGA | FSYYMDSVKGRFTISQDS | TTYALCRTWYTAA | TYWGQGT | Camelus |
| | SVQAGGSLRLS | | GKEREGVA | | SNTVYLEMDSLKPEDTA | AAYGY | QVTVSS | |
| | CKRSG | | AIA | | MYYCAA | | | |
| BAD00308.1 | AVQLVESGGG | FTYC | MAWFRQGP | SGGGT | YYFDDSMKPRFTISRETA | GSVVGCLGHTFG | TDWGQG | Camelus |
| | SVQAGESLKLS | | GKEREGVA | | KNTVHLQMTSLKPEDTAI | F | AQDTVSS | |
| | CTASL | | AID | | YYCAV | | | |
| BAD00313.1 | EVQLVESGGG | DDYRGFY | MGWFRQAP | IDIYNN | SGYADSAKGRFTISYDNV | ASDPVYYSATSD | YNWGQG | Camelus |
| | SVQIGGSLRLS | | GKEREGVG | ARP | KNTMYLQMSNLKAEDTG | RLIDYAY | TQVTVSS | |
| | CAASG | | SIA | | TYYCAA | | | |
| BAD00314.1 | AVQLVESGGG | VGFSFYT | MAWFHQAP | LDGT | TNYADSVKGRFTISRDNG | GVVSGTVVLDTC | AYWGQGT | Camelus |
| | SVQAGGSLRLS | | GKECELVSI | | KNTADLQMNSLILEDTGV | PPRSHES | QVTVSS | |
| | CTASG | | IS | | YYCAA | | | |
| BAD00315.1 | EVQLVESGGG | YSFSGYC | MGWFRQVP | TFGGT | TLYADSVKARFTISRDNA | CGPRSSFDNCAR | FYWGRGT | Camelus |
| | SVQAGGSLRLS | | GKEREGVA | | KNTMWLQMDSLKPEDTA | GESGY | QVTVSS | |
| | CAASG | | TIN | | IYYCAR | | | |
| BAD00319.1 | QVKLEESGGG | YTYCRYD | MSWYRQAP | GDSS | TSYADSVKGRFTISQDNA | ILNTIATMFDDAPV | GYWGQG | Camelus |
| | SVQAGGSLRLS | | GKEREFVS | | KNTVYLQMNSLIPEDTAM | QPCAF | TQVTVSS | |
| | CAASG | | AID | | YYCKT | | | |
| BAD00320.1 | AVQLVESGGAS | DWDSRKW | MAWFRKAP | TVDDS | TYYADSVKGRFAISRDNA | DSMIAGALNDRG | RFWGQGT | Camelus |
| | VETGGSLRLSC | | GSRREGVA | | KSTLYLQMNGLKPEDTA | Y | QVTVSS | |
| | AVSG | | SIY | | MYYCAS | | | |
| BAD00321.1 | AVQLVESGGG | AAEFRYC | ISWFRQAP | RDGI | TTYADSVKGRFTISQNNA | NPPSGSRACPY | DVWGQG | Camelus |
| | SVQAGGSLRLS | VYT | GKEREFVS | | KNTVFLQMDSLQPGDTA | | TQVTVSS | |
| | CVASP | | SIN | | MYFCKT | | | |
| BAD00323.1 | QVKLEESGGG | LISC | MAWFRQAP | TPVSS | TFYSDSVKGRFTISQDNA | PGKLYYGCDFWS | TYWGQGT | Camelus |
| | SVQTGGSLRLS | | GKEREEVA | | TNTVYLQMDSLKPEDTA | GY | RVTVSA | |
| | CVASE | | AIY | | MYYCAA | | | |
| BAD00325.1 | AVQLVESGGG | YAAT | LGWFRQAP | RDGF | TKYGDSVKGRFTVSRDN | AFLVWGLGDCPD | GAWGQG | Camelus |
| | SVQPGESLRLS | | GKECELVAT | | NKNTMYLEMTSLKPDDA | DRA | TQVTVSS | |
| | CLVSG | | IS | | GVYYCAH | | | |
| BAD00326.1 | QVQLVESGGG | LSLTHFC | MGWFRQD | PPVDT | TYYADSAKGRFTISRDNA | CSVCSGLSCGRP | VHWGQG | Camelus |
| | SVQAGGSLRLS | | PGKEREEV | | KNAVYLQMNSLKPDDTA | | TQVTVSS | |
| | CIASG | | ATIH | | TYYCAA | | | |
| BAD00328.1 | EVQLVESGGG | YTHCDYD | MSWYRQAP | SDGS | VNYADSVKGRFTISQDNA | HGVTWSASRGGR | NFWGQGT | Camelus |
| | SVQAGGSLRLS | | GKEREFVS | | KNTVYLQMNSLKPEDTA | WQDCQSGL | QVTVSS | |
| | CAVSG | | SFD | | MYYCKI | | | |
| BAD00329.1 | EVQLVESGGG | FPNNVNC | VAWVRQGP | TYGS | PYLADSVKGRFTVSLDRP | GTCHDEDGTMKL | AYWGRGT | Camelus |
| | SVQAGGSLRLS | | GKEREGVA | | ENTVYLEMNNLKPEDTGT | NVKGHD | QVTVSS | |
| | CVASE | | GIY | | YICAA | | | |
| BAD00330.1 | QVQLVESGGG | YGYCAYD | MSWYRQTP | SDGS | TSYADSVKGRFTISRDNA | RLRVSVFGCPMG | NYWGQG | Camelus |
| | SVQAGGSLRLS | | GKEREFVS | | KNAVYLQMNSLKPEETA | GN | TQVTVSS | |
| | CAASG | | GID | | MYYCKI | | | |
| BAD00331.1 | QVQLVESGGG | YGYSTDC | MGWFRQAP | GTSI | TYYADSLKGRFTISRDNA | EAGVYPYCSGAW | NYWGQG | Camelus |
| | SVQAGGSLRLS | | GKEREGVA | | RNTLYLQMNSLKPEDTA | DRRSAY | TQVTVSS | |
| | CAASG | | AIH | | MYYCAL | | | |
| BAD00332.1 | EVOLVESGGG | YTFSSGT | MGWFRQAP | SDGD | TNYADSVKGRFTISRDNA | PLTLSRYDCYSSS | GYWGQG | Camelus |
| | SVQAGGSLRLS | | GKGCELVS | | KNTVYLQMNSLKPEDTAV | RSNPL | TQVTVSS | |
| | CAASG | | STI | | YYCAA | | | |
| BAD00333.1 | AVQLVESGGG | YDHCTYD | MSWYRQAP | SDDM | ITYAESVKGRFTISRDNAK | TTAHCSGGECCG | HYWGKGT | Camelus |
| | SVQAGGSLRLS | | GKEREFVS | | STVYLQMNSLKPEDTATY | SFQM | LVTISS | |
| | CAASG | | TID | | YCKS | | | |
| BAD00334.1 | QVKLEESGGG | DTYSSHC | MGWFRQAP | GGL | TYYADSVKGRFTISQDNR | RGGGCWYPWWD | NYWGQG | Camelus |
| | SVQAGGSLRLS | | GKEREGVA | | KNTVYLQMTSLKPEDTA | AY | TQVTVSS | |
| | CAASG | | AIG | | MYYCAA | | | |
| BAD00338.1 | QVQLVESGGG | CTYCRC | MGWFRQAP | RESGT | TYSADSVKGRFTISQDNG | VPTSQWAACPPE | RYWGQG | Camelus |
| | SVQAGGSLRLS | | GKEREIVAR | | KNTVYLHMNTLKPEDTAI | Y | TQVTVSS | |
| | CVASG | | IN | | YYCAA | | | |
| BAD00339.1 | QVQLVESGGG | FTFNRCG | MAWYRQAA | TDGG | GSYADVVKGRFTISQDKA | QLSPEFGKQCA | SNWGQG | Camelus |
| | SVQAGGSLRLS | | GKEREWIS | | KDKVYLQMNNLKPEDTA | | TQVTVSS | |
| | CTAPG | | SIN | | MYSCKT | | | |
| BAD00340.1 | QVQLVESGGG | TYTTDS | MAWFRQTP | TSSGR | TSYADPVKGRFTISQDNA | SYYRDDHSADLD | HLWGQGT | Camelus |
| | SVQAGGSLRLS | | GKKREGVA | | KNTVYLQMNNLKPEDTG | AAEF | QVTVSS | |
| | CAVSG | | RIY | | MYYCAL | | | |
| BAD00341.1 | EVQLVESGGG | SYNMHC | MGWFRQTT | TGAGE | VWYVDSVKGRFTISHDN | DIAPCSGGYCVC | NIWGQGT | Camelus |
| | SVQAGGSLRLS | | GEEREGLA | | AKKMLYLEMNNLQPEDT | STRQKKY | QVTVSS | |
| | CETLE | | SIY | | ATYYCAA | | | |
| BAD00342.1 | EVQLVESGGG | FTFDLAD | MAWYIQPP | SEGV | TYYADAVKGRFTISRDNA | DMGGLWSEWGV | EYSGQGT | Camelus |
| | SVHAGGSLRLS | | GNECELVS | | KNTVYLPMNSLKPEDTA | PSSHCIDF | QVTVSS | |
| | CTTSG | | TIN | | MYYCAA | | | |
| BAD00344.1 | DVQLVESGGG | YTGSTGYC | IAWFRQAP | SSGGS | RYSADSVKGRFTLSQDN | RRYDCFATSWAY | NYWGQG | Camelus |
| | SVHAGGSLRLS | | GKEREGVA | | AKNTVYLHMNSLKPEDTA | EPAY | TQVTVSS | |
| | CAVSG | | TIN | | IYYCAA | | | |
| BAD00347.1 | EVQLVESGGG | YALGDVC | VGWFRQAP | TTYPT | TVTSDSVKGRFTISQDRIK | KSRFVKPCAPAS | TYWGQGT | Camelus |
| | SVQAGGSLRLS | | GKEREGVA | GGGA | NTVYLQMNSLNPEDTAM | DF | QVTVSS | |
| | CTTSN | | AIY | | YYCAA | | | |
| BAD00348.1 | DVQLVESGGG | FTIGHSV | MGWFRQAP | SDGR | TDYVDSVNGRFTVSRDN | LYLIPHLGAPVQC | GDWGQG | Camelus |
| | SVQAGGSLRLS | | GKECELVST | | ADNTIHLQMNSLKPEDTA | RRAF | TQVTVSS | |
| | CAASG | | IF | | VYYCAT | | | |
| BAD00350.1 | AVQLVESGGG | DIDRIRC | MAWFRQVP | TTNSY | TYYQDSVKGRFTISRDSA | ANRCTLGSWEGF | MYWGQG | Camelus |
| | SVQAGGSLRLS | | GKEREGVA | | GITLFLQMNSLQPEDTAM | | TQVTVSS | |
| | CVTAG | | GIL | | YYCEA | | | |
| BAD00351.1 | DVQLVESGGG | YTFCKYA | MTWYRQAP | SDGS | INYADSVKGRFTISQDNA | GFSPYCTDG | PYQGQGT | Camelus |
| | SVQAGGSLRLS | | GKDREFVS | | KNTVYLQMNSLKPEDTA | | QVTVSS | |
| | CAASG | | GIE | | MYYCKT | | | |
| BAD00352.1 | QVQLVESGGG | FTTACRY | MSWYRQAP | WDGS | TVYADSVKGRFTISQDNA | DSGSYRTFWCRD | VHWGQG | Camelus |
| | SVQAGGSLRLS | D | GKEREFVS | | KSTVYLQMNNLKPEDTA | DN | TQVTVSS | |
| | CSVSG | | SIH | | MYYCKI | | | |
| BAD00353.1 | QVQLVESGGD | YTIRPYC | MGWFRQAP | IGGTI | PFYADSVKGRFTISQDTA | RSGFCRREFEHD | DNWGQG | Camelus |
| | SVQAGGSLRLS | | GKEREGVA | | KNTVYLQMDSLLPKDTAI | AY | TQVTVSS | |
| | CAASG | | VMT | | YYCAA | | | |
| BAD00354.1 | EVQLVESGGG | VTNSRYS | IGWFRQAP | TGGM | TNYVTSVKGRFTISQDNA | GYRSYYRDYALL | NSWGQG | Camelus |
| | SVQAGGSLRLS | | EKEREAVA | | KNTVYLQMNSLKPDDTAI | SDGY | TQVSVSS | |
| | CVVSG | | AIN | | YYCAL | | | |
| BAD00355.1 | AVQLVESGGG | STYS | VAWFRQTP | TNHRV | KYYGDSVKGRFTISQDNT | KPSYQSGHWSVG | NFWGQGT | Camelus |
| | SVQDGGSLRLS | | GKEREGVA | | KSTVYLQMNSLKPEDTG | NLY | QVTVSS | |
| | CVVSG | | VIF | | MYYCAA | | | |
| BAD00356.1 | QVKLEESGGG | LMYREYY | MGWFRQAP | APSNS | AQYANSVKGRFTISRDKA | GRGRFRTGLDAV | DAWGQG | Camelus |
| | SVQAGGSLRLS | | GKEREGVA | | KNVMYLQMNNLKPEDTAI | EF | TQVTVSS | |
| | CAASG | | VIS | | YYCAQ | | | |
| BAD00357.1 | AVQLVESGGG | NIYS | MAWFRQAP | LGSII | QYYADSVKGRFTASQDR | REEYTPGHWAQT | HNWGQGI | Camelus |
| | SVQAGGSLRLS | | GKQREGVA | | DKRTMYLQMNSLEVEDT | STY | QVTVSS | |
| | CVASG | | TVY | | GVYYCAV | | | |
| BAD00360.1 | QVQLVESGGG | YTYRRIY | MAWFRQAA | RDIYTG | THYADSAKGRFTVSQDN | GPLYSGLWYRPS | EFWGQGT | Camelus |
| | SVQAGGSLRLS | | GKEREGIAA | HTN | AKTTVYLQMNSLEPEDTG | SY | QVTVSS | |
| | CVASG | | AY | | MYYCAA | | | |
| BAD00361.1 | QVQLVESGGG | RVYS | MAWFRQAP | PGSII | TYHADSLKDRFTASQDR | REYYSPGHWAVE | RNWGQGI | Camelus |
| | SVQAGGSLRLS | | GKQREGVA | | AKRTMYLQMNSLKVEDT | STY | QVTVSS | |
| | CVASG | | TVY | | GMYYCAV | | | |
| BAD00363.1 | QVQLVESGGG | NTWYS | VGWFRLAP | RGSFI | TYYADSVKGRFTISHDNA | KPVYKSGHWTDD | TYWGQGT | Camelus |
| | SVQAGGSLRLS | | GKEREGVA | | KNTVYLQMNSLKSEDTAI | RGY | QVTVSS | |
| | CVASA | | TIY | | YYCAS | | | |
| BAD00365.1 | QVQLVESGGG | LNYKGYY | IGWFRQAP | SPSNW | PEYANSVKGRFTISRDRA | GRGRFKTGLNAA | DAWGQG | Camelus |
| | SVQAGGSLRLS | | GKEREGVA | | ENIVDLQMNSLKPEETAIY | EY | TQVAVSS | |
| | CVVSG | | VIS | | YCAQ | | | |
| BAD00366.1 | QVQLVESGGG | RPECSYD | ISWYRQAP | TTGRT | TRYAEAVKGRFTISRDKA | EGATLICRDY | SYWGLGT | Camelus |
| | SVQAGGSLRLS | | GKEREFVA | SVDGR | KNTVYLQMNSLQPDDTA | | QVNVSS | |
| | CLRSG | | VIE | | MYYCHA | | | |
| BAD00368.1 | EVQLVESGGG | YVYS | TAWFRQTP | RDVGI | TYYADSMKGRFAISQDNP | KPTYQGGPWWK | NSWGQG | Camelus |
| | SVVAGGNLRLS | | GQEREGVA | | KNTVYLELHNLQPQDTA | PDGY | TQVTVSS | |
| | CVASG | | TIY | | MYYCAA | | | |
| BAD00369.1 | EVQLVESGGG | SVYS | MAWFRQAP | PGSRI | QYYADSVKDRFTASQDR | REEYAPGHWAQT | HNWGQGI | Camelus |
| | SVQAGGSLRLS | | GKQREGVA | | AKRTMYLQMNSLEVEDT | STY | QVTVSS | |
| | CVASG | | TVY | | GMYYCAV | | | |
| BAD00370.1 | AVQLVESGGG | NMNSWYS | MVWFRQAP | RDFDI | TYYADSVKGRFTISRDNT | KPVYQGGPWYDE | TYWGQGT | Camelus |
| | SVQAGGSLRLS | | GKEREGVA | | KNTVYLQMNNLKSEDTAI | AGY | QVTVSS | |
| | CVLSG | | TIY | | YYCAA | | | |
| BAD00372.1 | AVQLVDSGGD | YTGNSYC | LGWFRQPP | SGGP | NTYYADSVKGRFTIPQGS | ARLPEEGTCSNL | EYWGQGT | Camelus |
| | SVQAGGSLRLS | | GKEREWVA | | TRLVYLQMNSLKPEDTAI | RFSSDDF | QVIVSS | |
| | CVASG | | AKD | | YYCAA | | | |
| BAD00373.1 | AVQLVESGGG | RTYS | MAWFRQVP | TEASI | TYYAASVEGRFTISLDRT | RIVYNPGHWAQV | NTWGQGI | Camelus |
| | SVPAGGSLRLS | | DKEREGVA | | KSTVYLQMNSLKPEDSGI | STY | QVTVSS | |
| | CAASG | | TAY | | YYCAS | | | |
| BAD00374.1 | EVQLVESGGG | GTYS | TAWFRQAP | SVPGI | QYYADSVKGRFTVSRDN | KKSYGSGHWSMT | DYWGQG | Camelus |
| | SVQAGGSLRLS | | GREREGVA | | AKNTIYLQMNTLKPSDTA | SWY | TQVTVSS | |
| | CVASE | | VTY | | MYYCAA | | | |
| BAD00376.1 | EVPLVESGGGL | FSISSCC | MKWYRQAP | SAANT | LPYADFPVGRFAISRDGA | YCTSGGTCGPPN | NYRGQGT | Camelus |
| | VQAGGSLRLSC | | GKERELVS | YY | KNILYLQMNSLKPEDTAV | FN | QVTVSS | |
| | VASG | | SIT | | YYCAA | | | |
| BAD00377.1 | AVQLVESGGG | KTYC | MGWYRQA | PDYN | VRYYGTSVEGRFSISKGN | REEYGQCATSNR | SSWGQGT | Camelus |
| | SVQGGGSLTLS | | PGKEREGV | | NREVLLQMNSLKPEDTA | PNPDGF | QVTVSS | |
| | CVASG | | AVIY | | MYYCAA | | | |
| BAD00379.1 | AVQLVDSGGG | YSSVTFC | VGWFRQVP | SGGGS | VHYTDSVKGRFAISQDNG | AATNYGCDRLQY | NYWGQG | Camelus |
| | SVQAGGSLRLT | | GKERERVA | | ANTVHLVMNNLKPEDTAI | W | TQVTVSS | |
| | CAVSG | | TIN | | YYCAA | | | |
| BAD00380.1 | EVQLVESGGD | NSYS | MAWFRQAP | TDYNL | AYAADSVKGRFTISQDRA | RLQYGLGLHWSD | DYWGQG | Camelus |
| | SVQTGGSLRLS | | GKEREGVA | | KNTVYLQMNSLKPEDTA | TAAY | TQVTVSS | |
| | CVNSA | | TIY | | MYYCAT | | | |
| BAD00382.1 | DVQLVESGGG | SEYIYS | MAWFRQAP | AVADI | TYVADSVKDRFIISRENAE | RMAYSPDHWQS | QHWGQG | Camelus |
| | SVQAGGSLRLS | | GSPREGVA | | NTVALVMSDLKAEDSAM | SSAY | TQVTVSS | |
| | CTAST | | TIY | | YYCAA | | | |
| BAD00383.1 | QVQLVESGGG | KAYS | MAWFRQAP | TDVPG | TYYADDVKGRFAISQDRA | RLQYGKGLHWTN | NYWGQG | Camelus |
| | SVQAGGSLRLS | | GKEREGVA | V | KNTIYLQLNSLKPEDTGM | SDAY | TQVTVSS | |
| | CVNSV | | TIY | | YFCAT | | | |
| BAD00384.1 | QVKLEESGGG | YIFSNKN | MAWFRQAP | TGSGS | AYYADSVKGRFTISRDNA | DLSGRMRVSRVL | TYWGQGT | Camelus |
| | SVQAGGSLRLS | | GKEREGLA | | KNTVYLQMNSLKPEDTA | VATSY | QVTVSA | |
| | CATSG | | AIY | | MFFCAG | | | |
| BAD00386.1 | AVQLVESGGG | YIYSN | MGWFRQAP | TGAGT | TYYADSVKGRFTISRDNN | TRPYWSRSDYLD | NYWGQG | Camelus |
| | PVQAGGSLTLS | | GKEREGVA | | AKNMLYLHMNSLKPEDT | PRDY | TQVTVSS | |
| | CAASG | | ATN | | AIYYCA | | | |
| BAD00388.1 | AVQLVESGGG | YTYS | MGWFRQAP | TGGLR | TYYADSVKGRFTISRDNY | KDRRELIFRPSEA | NYWGQG | Camelus |
| | SVQAGGSLRLS | | GKEREGVA | | KNTVYLQMNSLKPEDTAI | GTY | TQVTVSS | |
| | CEASG | | AIS | | YYCAS | | | |
| BAD00391.1 | AVQLVESGGG | FTYSDSS | LAWFRQVP | TTSGD | TYHAASVKGRFTISQDKA | KRPWYGGTSEFF | NAWGQG | Camelus |
| | SVQAGGSLRLS | | GKEREGVA | | NHRVYLQMNSLKPEDTA | KEKNY | TQVTVSS | |
| | CAASG | | RIY | | MYFCAA | | | |
| BAD00393.1 | DVQLVESGGG | FTVSDCW | MNWFRQAP | GDGSR | TEYSESVKGRLTISKDNA | DPHDGFWFF | DHWGQG | Camelus |
| | LVQPGGSLRLS | | GKGLEWVS | | KNTLYLQMNTLRPDDTAV | | TQVTVSS | |
| | CAASG | | KIK | | YYCAR | | | |
| BAD00396.1 | EVQLVESGGG | NTFCSLD | MSWYRQAP | VGGGT | TNYADSVKGRFTISRDNA | VGL | GYWGQG | Camelus |
| | SVQAGESLRLS | | GKEREFVS | | KNTLYLQLNSLKTEDTAM | | TQVTVSL | |
| | CAASG | | NID | | YYCAN | | | |
| BAD00484.1 | EVQLVESGGG | YIGSFQC | VGWFRQSP | SDGAA | TYYADPVKGRFTISRDNA | WVLDYAM | DNWTKGT | Camelus |
| | SVQAGGSLRLS | | GKERELVS | | ELTLNLQMNSLKPEDTAV | | LVTISS | |
| | CAASG | | GIT | | YYCAN | | | |
| BAD00501.1 | AVQLVDSGGG | FTFSGTL | MSWFRQAP | TDGGN | TQYCDSVKGRFTISRDNA | AGGAFLPT | SFWGQGT | Camelus |
| | LVQPGGSLRLT | | GKGLEWVS | | KNTLFLQMNGLRPEDTA | | QVTVAS | |
| | CLASG | | GQH | | RYYCVI | | | |
| BAD00506.1 | QVQLVESGGD | FTFSSYD | MSWFRQAP | NRGSV | ADYLDSVKGRFTISRDNA | TASTTMYVPS | NYWGQG | Camelus |
| | LVPPGGSLRLS | | GKGLEWVS | | KNTLHLQMDGVQPEDTA | | TQVTVSS | |
| | CAASG | | GIW | | VYYCAT | | | |
| AAQ19976.1 | DVQLQASGGG | DLFSIVA | MGWYRQR | EGGG | TNYAYDVHGRFTISRDNA | LRGQYRRSEY | DYWGAGT | Lama |
| | LVQAGGSLRLS | | PGKQRELV | | KNTVYLQMNNPNPDDTA | | QVTVSS | |
| | CTASG | | AGIT | | VYYCNI | | | |
| AAQ19977.1 | DVQLQASGGG | SFSRFNV | VGWYRQAP | SGGS | TNYSPSVKGRFTISRDNA | VHTRVIIQQ | SYWGQGT | Lama |
| | LVQAGESLRLS | | GRQRELVA | | KNTVYLQLNSLKPDDTAV | | QVTVSS | |
| | CGGFG | | NMN | | YYCRY | | | |
| AAQ19978.1 | DVQLQASGGG | RSISTYG | MGRFRQTP | WSGGN | EYYADSVKGRFTISRDKS | RAIGNYGSRDED | GSWGQGI | Lama |
| | LVQAGDSLRLS | | GKEREFVA | | KNIVYLRMNSLKTEDTAV | F | QVTVSS | |
| | CTASG | | AIS | | YYCAA | | | |
| AAQ19979.1 | DVQLQASGGG | RMYTIND | MGWYRQA | VGGT | TNYKDSVKGRFTISRDNA | DIRDTLWDSR | LYWGQGT | Lama |
| | VVQPGGSLRLS | | PGNQRELV | | KKTVCLQMNSLKPDDTA | | QVTVSS | |
| | CAASG | | ARIS | | VYYCNA | | | |
| AAQ19980.1 | DVQLQASGGG | RTFSMYA | MAWFRQAP | WSGNT | TRYADSLKGRFIISRDNAA | RSRYSSDF | DQWGQG | Lama |
| | LVQAGASLRLS | | GKEREFVA | | KTVDLQMDKLKPEDTAAY | | TQVTVSS | |
| | CAASG | | AIN | | ICAD | | | |
| AAQ19981.1 | DVQLQASGGG | RTFTTGG | AAWFRQAP | RSGGG | TYYADSAKGRFIISRDNA | RRSVSWGAPSN | DYWGQG | Lama |
| | LVAVGDSLRLT | | GKEREFVA | | KNTGYLQMDSLKPEDTA | WYLTREY | TQVTVSS | |
| | CMASG | | AIS | | VYYCAR | | | |
| AAQ19982.1 | DVQLQASGGG | RIDTTYA | VAWFRQAP | WRGS | TYVANSVKDRFSVSRDN | KDVRGY | GYWGQG | Lama |
| | KVQAGDSLRLS | | GKEREFVA | | AKNTVYLQMNSLKPEDTA | | TQVTVSS | |
| | CAASE | | AIH | | VYYCYM | | | |
| AAQ19985.1 | DVQLQASGGG | RTFNTYV | VGWFRQAP | SSGGD | VHYASSVTGRFIMSRDDA | RNAYTFSALEHQ | PYWGQG | Lama |
| | TVQPGASLKLA | | GQGREFVA | | QNTEYLEMNGLKIEDTAV | Y | AQVTVSS | |
| | CKVSG | | TFS | | YYCAL | | | |
| AAL40827.1 | EVQLQASGGG | SLSRITV | MGWYRQA | SSGG | TDYADSVKGRFTISKDNA | KSRDSAGLSW | DYWGQG | Lama |
| | LVQPGGSLRLS | | PGKQRELV | | KALMYLQMTSLRPEDTAV | | TQVTVSS | |
| | CAASG | | AIIT | | YYCVG | | | |
| AAQ19988.1 | DAQLQASGGG | STSSINS | IGWYRQAP | GGGS | TVYADSVKGRFTTSSDKP | GTVPGRW | DYWGPGT | Lama |
| | LVHAGGSLRLS | | GKQRELVA | | KNIEYLQMNSLKPEDTAV | | QVTVSS | |
| | CAASG | | GFA | | YYCSA | | | |
| AAQ19989.1 | DVQLQASGGG | LTSRKYA | VGWFRQAP | WSGYS | HFYSEGARGRFTMSRDN | DPGVSEYALGLE | KYWGQGT | Lama |
| | SVQAGDSLTLS | | GQEREYIGT | | AKNTASLRMNSLKPSDTA | Y | QVTVSS | |
| | CAASG | | IS | | VYYCAA | | | |
| AAQ19990.1 | DVQLQASGGG | RTFSTYA | MGWFRQAP | WSGVS | TYYADSVKGRFTISRDNA | GSPL | NYWGQG | Lama |
| | LVQAGASLRLS | | GKEREFVA | | KNTVYLQMNSLKPEDTAV | | TQVTVSS | |
| | CAASG | | TIS | | YYCNV | | | |
| AAV66930.1 | QVKLQESGGG | GAFSTYD | LGWYRQAP | RGGHA | THYADSVEGRFTISRDNA | VGPPLRGWNLAY | HYWGQG | Lama |
| | LVQAGASLRLS | | GKEREFVA | | KNTMYLQMDSLSPEDTAI | | | |
| | CVDSG | | AII | | YYCNA | | | |
| AAV66932.1 | QVKLHSSGGG | SIFRITE | MGWYRQA | RDHS | TQYAESVTGRFTISRDVA | MHLTAVYVRQ | PVWGQG | Lama |
| | SVQPGGSLTLT | | PGSQREMV | | KNTVYLEMNNLKPDDTAV | | | |
| | CSASG | | AFVS | | YTCHA | | | |
| AAV66938.1 | QVQLQESGGG | ATFSINR | MGWYRQA | SRDD | TDYADSVKGRFTIWRDD | GAFIRLSRKTY | DYWGQG | Lama |
| | MVQLGGSLKLS | | PGKEREMV | | ANNTVYLQMVNVKAEDT | | | |
| | CVASG | | ARIT | | AIYYCNV | | | |
| AAV66950.1 | DVQLQASGGG | SIFSIGL | MRWYRQAP | SGGN | INYLNSVKGRVTISRDNA | EVRTTAVGSAF | AFWGQG | Lama |
| | LVQPGGSLSLS | | GKQREWIA | | KNTVYLQMNSLMPEDTA | | | |
| | CAASG | | TID | | VYVCNA | | | |
| AAV66951.1 | QVKLQQSGGG | LTFSTYD | WGWYRQA | WTGGS | LYYADSVKGRFTISRDNA | PIATQNP | ASWGQG | Lama |
| | LVQAGGSLRLS | | PGKGREPV | | KNTVYLQMNSLKPEDTAI | | | |
| | CAASG | | AVID | | YYCNA | | | |
| AAV66952.1 | QVKLQESGGR | RTFSTYD | MGWYRQA | SSGSS | TYYADSVKGRFTISRDNA | ELQRLNP | GSWGQG | Lama |
| | LVQAGGSLRLS | | PGKEREVV | | KNTVYLQMNSLKPEDTAI | | | |
| | CAASG | | AVIS | | YYCNA | | | |
| ABC47604.1 | GGGLVQAGGS | RTFYNYA | MGWFRQAP | WSGGS | TLYTDSVKGRFTISRANG | DADAQPMGIIENY | DYWGQG | Lama |
| | LRLSCAASG | | GKEREFVA | | QNTVYLQMNSLKPEDTA | | | |
| | | | AIS | | VYYCAA | | | |
| ABQ52434.1 | EVQLVESGGGL | SIFSLT | MGWYRQA | PGGD | TNYADFVKGRFTISRDNA | RTRNWG | TVWGQGT | Lama |
| | VQPGGSLRLSC | | PGKERELVT | | RSIIYLQMNSLKPEDTAVY | | QVTVSS | |
| | SFPG | | SAT | | YCYA | | | |
| ABQ52435.1 | EVQLVESGGEL | LTFSSYN | MGWFRRAP | WSGRD | TFYADSVKGRFTISRDNA | NPWPVAAPRSG | TYWGQGT | Lama |
| | VQAGGSLRLSC | | GKEREFVA | | KNTVYLQMSSLKPEDTAV | | QVTVSS | |
| | AASG | | SIT | | YYCAA | | | |
| ABQ52436.1 | EVQLVESGGGL | SIFSFA | MSWYRQAP | SDDR | VTYADSVKGRFTISRDNIK | QTDLRDWTVR | EYWGQGT | Lama |
| | VQPGESLTLSC | | GKERELVA | | RTAGLQMNSLKPEDTAV | | QVTVSS | |
| | VVAG | | RIG | | YYCNA | | | |
| ABQ52437.1 | EVQLVESGGGL | SIFTFA | MSWYRQAP | TDDE | TMYKDSVKGRFTISRDNV | RTDYRDWTVR | EYWGQGT | Lama |
| | VQPGGSLTLSC | | RKERELVA | | KRTAGLQMNNLKPEDTA | | QVTVSS | |
| | VAAG | | RIG | | VYYCNA | | | |
| ABS29543.1 | EVQLVESGGGL | VTFTPYQ | MGWYRQA | TGGSR | TNYADFAKGRFTISRDDV | YYAMIGHA | RNWGQG | Lama |
| | VQAGGSLRLSC | | PGKQRALV | | KNTVYLQMNNLKPEDTA | | TQVTVSS | |
| | TSST | | ADIS | | VYYCNT | | | |
| ABS29544.1 | EVQLVESGGG | LTFTPYR | MAWYRQAP | SGDGR | TNYADFAKGRFTISRDNIK | FVSFVGIA | RSWGQG | Lama |
| | FVQAGESLTLS | | GKQRDLVA | T | NTVFLRMTNLKPEDTAVY | | TQVTVSS | |
| | CTSST | | DIS | | YCNT | | | |
| ABS29545.1 | EVQLVESGGGL | LTFTPYR | MGWYRQA | GGDGR | TNYADFAKGRFTISRDNV | YVAIVGHA | RSWGQG | Lama |
| | VQAGDSLTLTC | | PGKQRDLV | T | KNAVYLQMNNLKPEDTAI | | TQVTVSS | |
| | TSPT | | ADIS | | YYCNT | | | |
| ABS29546.1 | EVQLVESGGGL | LTFTPYR | MGWYRQTP | PGDGS | KNYAGFAQGRFTISRDNI | YVAFVGRA | RTWGQGT | Lama |
| | VQAGGSLTLSC | | GKQRDLVA | T | KNTVYLQMNDLKPEDTA | | QVTVTS | |
| | TSST | | DIS | | VYYCNT | | | |
| ABS29547.1 | EVQLVESGGGL | IDFNLNA | MGWFRQAP | SADT | TNYRDFVKGRFTISINDAK | PFPTPSH | YHWGQG | Lama |
| | VQPGGSLRLSC | | GKQRELVA | | NTLYLQMDSLKPEDTAVY | | TQVTVSS | |
| | QFSG | | AIT | | YCNT | | | |
| ABX79392.1 | QVKLEESGGGL | RDFSDYV | MGWFRQAP | RNGLT | TRYADSVKGRFTISRDND | NSAGTYVSPRSR | DYWGQG | Lama |
| | VQAGDSLRVS | | GKEREFVA | | KNMVYLQMNSLKPEDTA | EY | TQVTVSS | |
| | CAASG | | AIS | | VYYCAV | | | |
| ABX79398.1 | QVQLVESGGG | RDFSDYV | MGWFRQAP | RNGIT | TRYADSVKGRFTISRDND | NSAGTYVSPRSR | DYDGWG | Lama |
| | LVQAGDSLRLS | | GKEREFVA | | KNTVYLQMNSLKPEDTAV | | QGTQVTV | |
| | CVDSG | | AIS | | YYCAT | | | |
| ABX79396.1 | QVQLVESGGG | GTFSSYA | MGWFRQAP | GRSSI | RNYDDSVKGRFAISRDSA | DTVFRSFVVGNV | KEWGQGT | Lama |
| | LVQAGGSLTLS | | GKEREFVA | | KNTVYLQMNSLKPEDTAV | | QVTVSS | |
| | CAASG | | AIS | | YYCAA | | | |
| ABX79402.1 | QVQLVESGGG | SIFSLDA | WGWYRQA | SDGS | TSYADSVKGRFTISRDNA | RFQSLY | NSWGQG | Lama |
| | LVQPGGSLRLP | | PGKQREMV | | NNTFYLQMNSLKPEDTAV | | TQVTVSS | |
| | CAASG | | ALVG | | YYCYA | | | |
| ABX79400.1 | QVKLEESGGGL | SIFDIDA | WGWYRQA | STGS | TSYADSVKGRFTLSRDNV | RFDSLY | NSWGQG | Lama |
| | VQPGGSLRLSC | | PGKQREMV | | NNTMYLQMNSLRPEDTA | | TQVTVSS | |
| | AASV | | ALVG | | VYYCYA | | | |
| ABX79401.1 | QVKLEESGGGL | LPFGINA | IGWYRQGP | SDGR | TILEDSVKGRFTISRDNAK | EKGGSP | LYWGQGT | Lama |
| | VQVGGSLRLSC | | GNQRDLVA | | KTVYVQMNNLKPEDTAV | | QVTVSS | |
| | AHSG | | RIT | | YYCAA | | | |
| ACD81985.1 | QVQLEESGGG | RTSSNTF | VGWFRQAP | RSDDR | TYYAASVRGRFTISGDSA | TRTWLVTGQSDY | PYWGQGT | Lama |
| | LVQAGGSLRLS | | GKEREFVA | | KNVVALQMSSLRPEDTA | | QVTVSS | |
| | CAASG | | AIR | | VYYCAA | | | |
| ACG68627.1 | EVQLQASGGG | GTLSDYH | VGWFRRPP | WSGG | TSYTNSVKGRFIISRDNEK | KYRDGERPY | DYWGQG | Lama |
| | LVQAGDSLRLS | | GKERERVA | M | NIVYLQMTRLKPEDTAVY | | TLVTVSS | |
| | CEAAR | | AIS | | YCAA | | | |
| ACG68622.1 | EVQLQASGGG | RTFSSYD | MGWFRQAP | RSGTV | LAYADSVKGRFTISRDNV | DRWENIRRGSV | RSWGQG | Lama |
| | LVQAGGSLRLS | | GKEREFVA | | KNTGYLQMNSLKPEDTA | | TLVTVSS | |
| | CAASG | | VIS | | VYYCAT | | | |
| ACG68628.1 | EVQLQASGGG | GTLSDYH | VGWFRQPP | WSGG | TSYTNSVKGRFIISRDNAK | KYRDGERPF | DYWGQG | Lama |
| | LVQAGDSLRLS | | GKERERVA | M | NIVYLQMTRLKPDDTAVY | | TLVTVSS | |
| | CEAAR | | AIS | | FCAA | | | |
| ACG68629.1 | DVQLQASGGG | HTFSEIG | MGWFRQAP | WSGTS | TYYADSVKGRFTISRDNA | RIQARFLDSSAF | DYWGQG | Lama |
| | LVQPGGSLKLS | | GKVREFVA | | KNTMFLQMNTLRPDDTA | | TQVTVSS | |
| | CAASG | | SIS | | VYYCAA | | | |
| ACG68631.1 | EVQQASGGGL | RTETTSA | IAWFRQAP | ASGLG | INYSGTVKGRFTISRDAD | GFHYIAAIRRTTDF | HFWGPGT | Lama |
| | VQAGGSLRLSC | | GKEREFVA | | KTTVYLQMNSLTPEDTAV | | LVTVSS | |
| | AASG | | QIS | | YYCAA | | | |
| ACG68632.1 | EVQLQASGGG | GTLSDYH | VGWFRRPP | WSGG | TSYTNSVKGRFIISRDNEK | KYGGGERPY | DYWGQG | Lama |
| | LVQAGDSLRLS | | GKERERVA | M | NIVYLQMTRLKPEDTAVY | | TQVTVSS | |
| | CEAAR | | AIS | | YCAA | | | |
| ACJ09035.1 | GGGLVQAGGS | RTFSSYI | MAWFRQAP | WGGD | TYYADSVKGRFTVSRHN | NDNYCSGYGCYD | DYWGPG | Lama |
| | LRLSCVASG | | GKEREFVA | | AKNTVYLQMNSLKPEDA | YMGY | | |
| | | | ASS | | AVYYCAE | | | |
| ACN89786.1 | QVQLQDSGGG | RTFSSYA | MGWFRQPP | RSGS | AIYAYPVKGRFTMSRDNA | ARSGVPSSRPTD | DYWGQG | Lama |
| | LVQAGGSLRLS | | GKEREWVS | | KNTVYLEMNSLKPEDTAV | Y | TQVTVSS | |
| | CEASG | | TIS | | FYCAA | | | |
| AFN61318.1 | QVQLVQSGGG | RTFSSYA | MAWFRQAP | WSGAN | IYVADSVKGRFTISRDNA | KLGFAPVEERQY | DYWGQG | Lama |
| | LVQAGGSLRLS | | GKEREFVA | | KDTVYLQMNSLKPEDTAV | | TQVTVSS | |
| | CAASG | | AIS | | YYCAV | | | |
| AFN61319.1 | QVQLVQSGGG | SFFSINV | MGWYRQA | SDGN | TNYADSVKGRFTISRDNA | RIWSGL | DYWGQG | Lama |
| | LVQPGGSLRLS | | PGKERESV | | KNAVYLQMNSLKPEDTA | | TQVTVSS | |
| | CTASG | | AVIT | | VYFCNA | | | |
| AGH30286.1 | EVQLQASGGG | RTSSMYS | MVWFRQAP | WTSSL | TYYADSLKGRFTISRDNA | DTKTGGGGY | EYWGQVT | Lama |
| | LVQAGGSLRLS | | GKEREFVA | | KNTVYLQMNSLKPEDTAI | | VTVSS | |
| | CAASG | | GII | | YYCAA | | | |
| AGH30281.1 | EVQLQASGGG | SIFSSDV | MGWFRQAP | DDGG | TNYADSVKGRFTISRDNA | RYYSGGYR | NYWGQVT | Lama |
| | LVQPGGSLRLS | | GKERELVAF | | ENTVSLQMNSLKPEDTAV | | VTVSS | |
| | CAASG | | IT | | YYCNA | | | |
| AGH30284.1 | DVQLQASGGG | SIFSSDV | MAWFRQAP | DDGG | TNYADSVKGRFTISRDGA | RYYSGTYR | SYWGQGT | Lama |
| | LVQPGGSLRLS | | GKERELVA | | KSMVSLQMNSLKPEDTA | | QVTVTV | |
| | CAASG | | MIT | | VYYCNA | | | |
| AGH30289.1 | EVQLQASGGG | PTYDSYG | MAWFRQGP | LSHST | TYYADSVKGRFTISRGNA | GNLRIAVPPSSVG | DYWGQG | Lama |
| | LVQAGGSLRLS | | GQGREFVA | | ENTVYLQMNSLKPEDTAV | Y | TQVTVTV | |
| | CAASG | | AIS | | YTCAI | | | |
| AGH30297.1 | DVQLQASGGG | NIDTIDV | MGWYRQA | SQGS | TNYADSVKGRFTISRDNA | WILSTDHSYK | HYWGQG | Lama |
| | LVQAGGSLRLS | | PGKQRELV | | KNTVYLQMNNLEPEDTA | | TQVTVTV | |
| | CAASG | | ADIT | | VYYCAQ | | | |
| AGH30295.1 | EVQLQASGGG | NINTIDV | MGWYRQA | RLAS | ANYADSVKGRFTISRDNA | WILSTDHSYM | HYWGQG | Lama |
| | LVQAGGSLRLS | | PGKQRELV | | KNTVYLQMNNLEPKDTA | | TQVTVTV | |
| | CAASG | | ADIT | | VYYCAQ | | | |
| AGH30293.1 | EVQLQASGGG | RTDSTYA | MAWFRQAP | YTGGT | THYADSVKGRFTISRDVA | KRSSWYRPFGVD | GSWGQG | Lama |
| | LVQAGGSLRLS | | GKEREFVA | | KDIMYLQMNSLKPEDTAV | EF | TQVTVTV | |
| | CAASG | | AIT | | YYCAE | | | |
| AGH30294.1 | DVQLQASGGG | PIYESYG | MAWFRQGP | LSSST | TYYGDSVKGRFTISRDNA | GNLRIAVPPTSVG | DYWGQG | Lama |
| | LVQAGGSLRLS | | GQGREFVA | | QNMVYLQMNSLKPEDTAI | Y | TQVTVTV | |
| | CAASG | | AIH | | YFCAI | | | |
| AGH30298.1 | EVQLQASGGG | GTFSNPS | MGWFRQAP | WSAGS | TYYADSVKGRFTISRDNA | TYYNGEYYLLRAD | QHWGQG | Lama |
| | LVQAGGSLRLS | | GKEREFVA | | KNTVDLRMTSLKPEDTAV | RY | TQVTVTV | |
| | CAASG | | AVS | | YYCSA | | | |
| 4wem_B | QVQLQESGGG | NVDRIDA | MGWFRQAP | EGGI | LNYGDFVKGRFTISRDNA | SHWGTLLIKGI | EHWGKGT | Lama |
| | LVQAGGSLRLS | | GKQREFVG | | KNTVYLQMSNLKSEDTG | | QVTVSS | |
| | CEASG | | YIS | | VYFCAA | | | |
| 4wen_B | QVQLQESGGG | SISSINA | MGWYRQA | NTGV | TEFADSVKGRFTISRDNA | TDWGTLLIKGI | DHWGKGT | Lama |
| | LVQPGGSLRLS | | PGSKREFV | | KTTVDLQMNSLKPEDTAV | | QVTVSS | |
| | CTASG | | AHIT | | YYCAA | | | |
| 4weu_E | QVQLQESGGG | LTFDTYA | MGWFRQAP | WTGIS | TYYADIAKGRFTISRDNAK | QKSLNVPAPW | DYWGQG | Lama |
| | LVQAGGSLRLS | | GKKREYVA | | NTLYLQMDSLKPEDTAVY | | TQVTVSS | |
| | CAASG | | AIS | | YCAA | | | |
| AGN90987.1 | QVQLQESGGG | LTFDTYA | MGWFRQAP | WTGIS | TYYADIAKGRFTISRDNAK | QRSLNVPAPW | DYWGQG | Lama |
| | LVQAGGSLRLS | | GKKREYVA | | NTLYLQMDSLKPEDTAVY | | TQVTVSS | |
| | CAASG | | AIS | | YCAA | | | |
| 4b41_B | EVQLVESGGGL | SIISNNA | MGWYRQA | SGGR | TTYADSVKGRFTISRDNA | ASLVRGPL | DHWGQG | Lama |
| | VQPGGSLRLSC | | PGKQRELV | | KTTVYLQMNSLKPEDTAV | | TQVTVSS | |
| | AASR | | ARIS | | YYCNA | | | |
| 4b5e_B | EVQLVESGGGL | RSFSRDA | MGWFRQAP | LNGGR | TYSADSVKGRFTISRDND | REGDVGLVSYKR | PYWGQGT | Lama |
| | VQAGGSLRLSC | | GKERDVVA | | KNTVYLQMSNLKPEDTAV | SSNY | QVTVSS | |
| | AASG | | AIN | | YYCAA | | | |
| 5h8o_A | QVQLQESGGG | FTFSRYA | MSWYRQAP | SGGGT | IYYADSVKGRFTISREDAK | G | GFWGQG | Lama |
| | LVQPGGSLKLS | | GKERESVA | | NTVYLQMNSLKPEDTAVY | | TQVTVSS | |
| | CAASG | | RIS | | YCYV | | | |
| AHA50077.1 | EVQLQASGGG | RTFSDLS | MGWFRQAP | WSVGT | TYYADSVKGRFTISRDNA | TYYTGEYYLLQAD | QHWGQG | Lama |
| | LVQAGGSLRLS | | GKEREFVA | | KNTVDLQMISLKPEDTAV | RY | TQVTVTV | |
| | CAASG | | AVS | | YYCSA | | | |
| AHA50078.1 | EVQLQASGGG | RTFSDLS | MGWFRQAP | WSVGT | TYYADSVKGRFTISRDNA | TYYTGEYYLVQA | DRYQHW | Lama |
| | LVQAGGSLRLS | | GKEREFVA | | KNTVDLQMISLKPEDTAV | | GQVTVTV | |
| | CAASG | | AVS | | YYCSA | | | |
| AHA50079.1 | EVHLQASGGG | RTFSDLS | MGWFRQAP | WTPGT | TYYADSVKGRFTISRDNA | TYYTGEYYLVQA | QHWGQG | Lama |
| | LVQEGGSLRLS | | GKEREFVA | | KNTVDLQMISLKPEDTAV | DRY | TQVTVTV | |
| | CAASG | | AVS | | YYCTA | | | |
| AHH24256.1 | QVKLEESGGGL | LTFRNFH | MAWFRQVA | WSRDR | QYYPDPVKGRFTITRDNA | RTASASGDWYKG | QYWGQG | Lama |
| | VQAGGSRRLS | | GKEREVVA | | KNTVYLQMNSLKPEDTAV | SY | TQVTVSS | |
| | CATSG | | AIS | | YYCAA | | | |
| AHH24257.1 | QVKLEESGGGL | STFSINA | LGWYRQAP | SDGT | VYYTDSVKGRFTISRDNA | AGKRIGSDGSIWF | GSWGQG | Lama |
| | VQAGGSLRVS | | GKARELVA | | KNTVSLQMSSLKPEDTAV | AVASF | TQVTVSS | |
| | CTASV | | AIG | | YYCNA | | | |
| AHY24762.1 | QVQLVESGGG | RTFSTST | LGWFRQAP | WLGGR | TNYADSVKGRFTISRDSA | RRLGV | DYWGQG | Lama |
| | LVQAGGSLRLS | | GKEREFVA | | KNTLFLQLNSLKPEDTAV | | TQVTVSS | |
| | CAASG | | AIS | | YYCYA | | | |
| AHY24764.1 | QVQLVESGGG | SIFRFYA | MGWYRQA | RGGI | TNYADSVKGRFTISRDNA | VGPLGSTP | REWGQG | Lama |
| | LVQPGASLRLS | | PGKQRELV | | KNTVYLQMNSLKPEDTAV | | TQVTVSS | |
| | CAASR | | ASIT | | YYCNR | | | |
| AHY24765.1 | QVQLVESGGG | SIFRFYA | MGWYRQA | RGGI | TNYADSVKGRFTISRDNA | RRIGR | DYWGQG | Lama |
| | LVQPGGSLRLS | | PGKQRELV | | KNTVYLQMNSLKPEDTAV | | TQVTVSS | |
| | CAASR | | ASIT | | YYCYA | | | |
| AKQ22868.1 | EVQLVESGGGL | RTFSRTS | MGWFRQAP | WGRGS | TYYADSVKGRFTISRDNA | GQEGFYTLVPRN | SYWGQGT | Lama |
| | VQAGGSLRLSC | | GKEREFVA | | KNTVYLQMNSLKPEDTAV | SRNW | QVTVSS | |
| | AASG | | AIN | | YYCAA | | | |
| AKQ22869.1 | EVQLVESGGGL | FTVDNYA | IGWFRQAP | SGGW | KFYPDSMKGRFSISRDNA | DLTNHCGNYDKG | GSWGQG | Lama |
| | VQPGGSLRLSC | | GKEREGVS | | KNTVYLQMNSLKPQDTA | F | TQVTVSS | |
| | AASG | | CIS | | VYYCGT | | | |
| AKQ22870.1 | EVQLVESGGGL | NSLRIGT | MAWYRQAP | SGGS | IDYTDSVKGRFTISRDNA | RSYYSASGAMYT | VIWGQGT | Lama |
| | VQPGGSLRLSC | | GKQREMVA | | KNTVFLQMNSLKPEDTAV | | QVTVSS | |
| | AASG | | TVT | | YYCYA | | | |
| ARG41544.1 | EVOLVESGGGL | RTFSTYP | MAWFRQAP | RGGIT | SYYADSVKGRFTISRDNA | YKASATAYTRGR | VHWGQG | Lama |
| | VQAGGSLRLSC | | GKEREFVA | | KNTVYLQMNSLKPEDTAV | PDEI | TQVTVSS | |
| | AASS | | TIS | | YYCAQ | | | |
| ARG41545.1 | EVQLVESGGG | RYH | MGWFRQAP | SSGGS | TSYADSVKGRFTISRDSA | RSGLWMATSTQG | DYWGQG | Lama |
| | TVKAGGSLRLS | | GKEREFVT | | KSTVYLQMNSLKPEDTAV | HY | TQVTVSS | |
| | CAAPR | | AIS | | YFCAA | | | |
| ARG41546.1 | EVQLVESGGGL | FSLDNYG | IGWFRQAP | STSLT | RYYAASVKGRFTISRDNS | TDRWPGIEWYRG | DNWGQG | Lama |
| | VQPGGSLRLSC | | GKEREGVS | | KNTVYLQMNSLNPEDTA | EY | TQVTVSS | |
| | AASG | | YIS | | VYYCAS | | | |
| ATN23974.1 | QVKLQESGGG | AFFRAGP | MGWYRQA | RDRT | TDYADSVKGRFTISRDDA | LGT | GFFGQGT | Lama |
| | SVQEGGSLRLS | | PGRQRDLV | | KNTVYLQMNSLKSEDTAV | | QVTVSS | |
| | CAASG | | ALIR | | YFCNP | | | |
| ARG41548.1 | EVOLVESGGGL | RTFRSDV | MGWFRQAP | ENGDIL | TRFEGSAKGRFTISRDNA | RWGTITTVSHQY | HFWGQGT | Lama |
| | VQAGGSLRLSC | | GRGREFVA | | NDTVYLQMSSLKPEDTAI | | QVTVSS | |
| | APSE | | AIA | | YYCAA | | | |
| ARG41549.1 | EVQLVESGGGL | PTFSDYA | MAWFRQAR | WIGGS | TYYADSVKGRFTISRDSA | ARLGTYYGSYIRA | EYWGQGT | Lama |
| | VQTGGSLRLSC | | GKEREFVA | | KDTMYLQMNTLKPEDTA | YFNRGRY | QVTVSS | |
| | ARSG | | SIT | | DYYCAI | | | |
| ARG41551.1 | EVQLVESGGGL | RIFSVYR | VGWFRQAP | NRNGA | TFYADSVKGRFTISRDSA | RDQGTTEY | HYWGQG | Lama |
| | VQAGGSLRLSC | | GKEREIVAS | S | KNTVYLQMRSLRPEDTA | | TLVTVSS | |
| | AASG | | IN | | VYYCAA | | | |
| ARG41553.1 | EVQLVESGGGL | RYP | MGWFCQAP | RSGAS | TYYADSVKGRFTIARDSA | KENYYGDYGLAQ | DYWGQG | Lama |
| | VQAGGSLRLSC | | GGEREFVA | | KNTVYLEMNSLKPEDTAV | NY | TQVTVSS | |
| | AASG | | AIS | | YYCAA | | | |
| ARG41554.1 | EVQLVESGGGL | SSTYE | MGWFRQAP | WNGG | TYYADSVKGRFTISRDNA | AYGLRDAFRMYR | PYWGQGT | Lama |
| | VQAGGSLRLSC | | GKEREFVA | R | KMTVYLQMNSLKPEDTA | SEY | QVTVSS | |
| | AASG | | GIN | | AYYCSA | | | |
| ARG41555.1 | EVQLVKSGGGL | RTFSSYA | MGWFRQAP | WNGG | TYYADFVKGRFTISRDNA | GHLTSTGVLHPA | DYWGQG | Lama |
| | VQVGGSLRLSC | | GKERELVA | R | KNTVYLQMNSLKPEDTAV | DYYNWDEY | TQVTVSS | |
| | AASG | | AIN | | YYCAA | | | |
| ARG41556.1 | EVOLVESGGGL | RYV | MGWFRQAP | RSGGS | TTYADSVKGRFTISRDSA | WSYYGVAYTSTT | DYWGQG | Lama |
| | VQAGGSLRLSC | | GQEREFVT | | ENTVYLQMNSLKPEDTAV | SF | TLVTVSS | |
| | ATSG | | SIS | | YYCAA | | | |
| ARG41557.1 | EVQLVESGGGL | FTLDYYA | IGWFRQAP | SSDGS | TYYADSVKGRFTISRDNA | DLKRRCRDYARP | DYWGQG | Lama |
| | VQPGGSLRLSC | | GKEREGVS | | KNTVYLQMNSLKPEDTAV | QRGN | TQVTVSS | |
| | AASG | | CIS | | YYCAT | | | |
| ARG41559.1 | EVQLVESGGAL | RTFSHYA | MGWFRQAP | WDGDS | TSYANSLKGRFTISRDNA | GPNFSTLARRY | DYWGQG | Lama |
| | VQAGDSLRVS | | GKQREFVA | | KNTGYLYMNSLIPEDTAV | | TLVTVSS | |
| | CVASG | | AIS | | YYCAA | | | |
| ARG41560.1 | EVQLVKSGGGL | RVA | MGWFRQPP | ASGAT | KYYADSVKGRFTIFRDNA | RLRWDSDYTSGG | DYWGQG | Lama |
| | VQAGGSLRLSC | | GKEREFVA | | NTTVYLQMNSLKPEDTAV | RY | TQVTVSS | |
| | AASG | | AIS | | YYCAA | | | |
| ARG41562.1 | EVQLVESRGGT | RTFSNYA | AGWFRQAP | AKGGV | TYYVDFVKGRFTISRDNA | DGSYGTILATIAEN | KDWGQG | Lama |
| | VQAGGSLRLSC | | GKEREFVA | | KNTVYLQMNSLKPEDTAV | SRISLL | TLVTVSS | |
| | AASG | | GIS | | YYCAA | | | |
| ARG41563.1 | EVQLVESGGG | RIA | MGWFRQAP | ASGGS | TEYADSVKGRFTISRDSS | RLWNQREY | PYWGQGT | Lama |
| | FVQAGGSLRLS | | GKEREFVA | | KSTAYLQMNSLKPEDTAV | | QVTVSS | |
| | CAASV | | RIS | | YYCAA | | | |
| ARG41564.1 | EVQLVESGGGL | RISSTYR | MGWFRQAP | WSGG | VLYTDSVKGRFTISTDNG | RNYGTTEY | GYWGQG | Lama |
| | VQAGGSLRLAC | | GKEREFVA | G | KNMVYLQMNSLRPEDTA | | TQVTVSS | |
| | AASG | | ASR | | VYYCAG | | | |
| ARG41565.1 | EVQLVESGGGL | LTFSDYA | MGWFRQAA | WGGR | TYYGDFAKGRFTISRENS | KRGMADIAYTHAT | DYWGQG | Lama |
| | VQTGGSLRLSC | | GKEREFVG | H | KNTVYLQMNSLKPEDTAV | SY | TQVTVSS | |
| | AASG | | VIS | | YTCAR | | | |
| ARG41566.1 | EVQLVESGGGL | RYA | MGWFRQAP | KSGGS | TYIADSVKGRFTISRHNAK | NGFLGTSGRRVD | DYWGQG | Lama |
| | VQAGGSLRLSC | | GKEREFVG | | STAYLQMNNLRPEDTAV | QY | TQVTVSS | |
| | AASG | | VIS | | YYCAA | | | |
| ARG41567.1 | EVQLVESGGGL | FTLDNHA | IGWFRRAP | SSSGT | TAYADSVEGRFTISRDNA | RFQGGFLGCTFA | GYWGQG | Lama |
| | VQPGGSLRLSC | | GKERERIGC | | KNTVYLQMNSLKPEDTAV | PQTF | TQVTVSS | |
| | AASG | | IS | | YYCAA | | | |
| ARG41568.1 | EVQLVESGGGL | RYV | MGWFRQAP | RSGGS | TNYADSVKGRFSVSRDS | SNRYGTNVLVTTA | DYWGQG | Lama |
| | VQAGGSLRLSC | | GKEREFVA | | AKNTVYLQMNDLKPEDT | LY | TQVTVSS | |
| | EASG | | VIS | | AVYYCAA | | | |
| ARG41569.1 | EVQLVESGGGL | RTFSVYP | MGWFRQAP | RRGAI | SYYQDSVKGRFTISRDSA | YKASSSSYTRGR | VYWGQGT | Lama |
| | VQAGGSLRLSC | | GKEREFVA | | NDTVYLQMNSLRPEDTA | PDEI | QVTVSS | |
| | AASG | | TIS | | VYYCAQ | | | |
| ARG41570.1 | EVQLVESGGGL | GIFDNYA | MGWIRQAP | WSESS | TYYAASVKGRFTISRDNA | WFWISSTWSYFS | NYRGPGT | Lama |
| | VQAGGSLRLSC | | GKEREFVA | | KNTVYLQMNSLKPEDTAV | ENEP | LVTVSS | |
| | AASG | | GIR | | YSCAA | | | |
| ARG41574.1 | EVQLVESGGGL | IYFRLYT | MNWYRQAL | NDGS | TNYGASIKGRFTISRDNA | H | DYWGQG | Lama |
| | VQAGGSLRLSC | | GKQREYVA | | KNTVYLQMNNLNPEDTA | | TQVTVSS | |
| | VVSG | | MIT | | VYYCNA | | | |
| ARG41575.1 | EVQLVESGGG | RYR | MGWFRQAP | ASGGS | TYYADSVKGRFTIARDNA | KVNYYGDYDLAQ | DYWGQG | Lama |
| | SVQAGGSLRLS | | GKEREFVA | | KTMVYLQMNSLKPEDTA | NY | TQVTVSS | |
| | CAASG | | VIS | | VYYCAA | | | |
| ARG41576.1 | EVQLVESGGGL | SIFSVNA | MGWYRQA | SGRS | PAYADSVKGRFTISRDNA | KGEYYSRSQY | NVWGQG | Lama |
| | VQPGGSLRLSC | | PGKQRELV | | ENTVYLQMNSLKPEDTAV | | TQVTVSS | |
| | AASG | | ASMV | | YYCNI | | | |
| ARG41577.1 | EVQLVESGGGL | RSFSGDT | MGWFLQAP | WSSRS | TNYADSVKGRFTISRDNA | GPPSPYIYSRPDL | TYWGQGT | Lama |
| | VQAGGSLRLSC | | GKEREFVA | | KNTVYLQMNTLKPEDTAV | Y | QVTVSS | |
| | AASG | | GIN | | YYCAA | | | |
| ARG41578.1 | EVQLVESGGG | RVA | MGWFRQAP | ASGGS | SSYADSVKGRFTISRDSA | RARTLGWVSADV | DYWGQG | Lama |
| | SVQAGGSLRLS | | GKEREFVA | | KSTVYLQMNSLEPEDTAV | SAY | TQVTVSS | |
| | CAASG | | AIS | | YYCAA | | | |
| ATN23917.1 | KVKLQESGGG | SAFSINS | MYWYRQAP | SAGA | TTYASSLKDRFTISRDNA | GG | STWGQGT | Lama |
| | AVEIGGSLRLS | | GKVREVVA | | KNTVYLHMSSLKVEDTAV | | QVTVSS | |
| | CVASG | | MIT | | YYCDA | | | |
| ARG41580.1 | EVQLVESGGGL | NFFNIIT | MGWFRQAP | AGRS | ISYLDSVKGRFTIVRDNAK | TGHRSVTGGIY | VTWGQGT | Lama |
| | VQAGGSLRLTC | | GKERELVA | | NTVILEMNSLTPEDTAVY | | LVTVSS | |
| | AASG | | VDT | | YCYA | | | |
| ARG41582.1 | EVQLVESGGGL | RAFSSYA | MGWFRQAP | GRGGV | TYYAASVKGRFTISRDNA | GPNIGVLTDPAYS | GDWGQG | Lama |
| | VQAGGSLRLSC | | GKEREFVA | | KNTVLLQMSSLKPEDTGV | G | TLVTVSS | |
| | AASG | | VIS | | YYCAA | | | |
| ARG41583.1 | EVQLVESGGGL | RSFGSYH | MGWFRQAL | SGGGL | LDYEDSVKGRFTISRDNA | TARVWGSWFTSD | DYWGQG | Lama |
| | VQAGGSLRLSC | | GKEREFVA | | KNTVYLQMNSLTPEDTAV | Y | TLVTVSS | |
| | AASG | | AIS | | YYCAA | | | |
| ARG41584.1 | EVQLVESGGGL | RAYRNYV | MGWFRQG | PDDGS | ILSSNSVRGRITISSDNAK | GTTPSMLARKY | DYWGQG | Lama |
| | VQAGGSLRLSC | | PGKEREFV | | NTVYLQMSTLAPEDTAIY | | TLVTVSS | |
| | VASG | | ATIR | | YCAY | | | |
| ARG41587.1 | EVQLVESGGGL | RTFSTYA | LGWFRQAP | GRGGT | TYYAGSVKGRLTISRDNA | GSDFGAVVDNRP | DYWGQG | Lama |
| | VQAGDSLRLSC | | GKEREFVA | | KNTVYLQMNSLKPEDTAV | | TLVTVSS | |
| | AASG | | VIS | | YYCAA | | | |
| ARG41588.1 | EVQLVESGGGL | IPFSRYH | MGWFRQAP | WSGQN | TYYADSVKGRFTISRDNA | GSRYYTDIIPNYY | RYWGQG | Lama |
| | VQAGGSLRLSC | | GKEREFVA | | KNTGNLQMNSMKPEDTA | | TLVTVSS | |
| | AASG | | SVS | | VYYCAA | | | |
| ARG41589.1 | EVQLVESGGGL | DTFSTYG | MGWFRQAP | WNRR | TYYADSVKGRFTISRDNA | VRGDNLFHTRPR | DYWGQG | Lama |
| | VQAGGSLRLSC | | GKEREIVAR | | KNAAYLEMNSLKPEDTAV | DY | TLVTVSS | |
| | AASG | | IT | | YMCAA | | | |
| ARG41590.1 | EVQLVESGGG | RYA | MGWFRQVS | ASGGS | TYYADSVKGRFTISRDNA | TSGGIFRGLYYDT | NYWGQG | Lama |
| | VVQTGGSLRLS | | GKEREFVA | | KTTLAYLQMISLKPEDTAV | RNY | TLVTVSS | |
| | CAASG | | FIS | | YYCA | | | |
| ARG41594.1 | EVQLVESGGGL | RTFSIYS | MGWFRQAT | WSGTR | TYYADSVKGRFTISGDIAK | GGSRGYVAAPDR | DYWGQG | Lama |
| | VQAGDSLRLSC | | GKEREFVA | | NTVYLQMNNLKPEDTAV | W | TLVTVSS | |
| | AASG | | AIR | | YSCAA | | | |
| ARG41595.1 | EVQLVESGGGL | IYFRLYT | MGWYRQA | STRS | THYADSVKGRFTISRDNA | HNGQG | DFWGQGT | Lama |
| | VQTGGSLRLSC | | PGNQRELV | | KNTLYLQMNSLKPEDTAV | | LVTVSS | |
| | AASG | | ALIT | | YYCNA | | | |
| ARG41596.1 | EVQLVESGGG | RYG | VGWFRQPP | KSGGS | TYYEDSVKGRFTISRDGA | DGGGYSAYYYYT | EYWGQGT | Lama |
| | VVQAGGSLRLS | | GKEREFVS | | KNTVYLQMNSLKPEDTAV | HQTEY | LVTVSS | |
| | CAASG | | AVS | | YYCAA | | | |
| ARG41597.1 | EVQLVESGGGL | RYV | MGWFRQAP | RSGAT | TNYADSVKGRFTISRDSA | SGRYYTAAVEGA | DYWGQG | Lama |
| | VQAGGSLRLSC | | GKEREFVA | | KITVYLQMDSLKPEDTAV | NY | TLVTVSS | |
| | AASG | | AIT | | YYCAA | | | |
| ARG41598.1 | EVQLVESGGAL | RTFSRYA | MGWFRQAP | WDGDS | TSYANSLKGRFTISRDNA | DRGRTTAW | TYWGEGT | Lama |
| | VQAGDSLRVS | | GKQREFVA | | KNTGYLYMNSLIPEDTAV | | LVTVSS | |
| | CVASG | | AIS | | YYCAA | | | |
| ARG41599.1 | EVQLVESGGGL | GTLNGYA | MAWFRQAP | WSGS | KYYADSVKGRFTISRDNA | RRGGGNYYTRAV | DYWGQG | Lama |
| | VQAGDSLRLSC | | GKERDVVA | W | KGTVYLQMNSLKPEDTA | DF | TLVTVSS | |
| | AASG | | TIN | | VYRCAA | | | |
| ARG41601.1 | EVQLVESGGGL | RSFSRST | MGWFRQAP | WAGTT | TYYHDSVKGRFTISRDNA | GRDARILRNARDY | DYWGQG | Lama |
| | VQSGDSLRLSC | | EKEREFVAV | | KNTVYLQMNSLKPEDTAV | | TLVTVSS | |
| | AASG | | IS | | YYCAG | | | |
| ARG41605.1 | EVQLVESGGGL | GTFDDYA | MGWFRQR | WNGA | TYYLNHIKGRFTISRDNAK | ERTLGWGVNPEA | ASWGQGT | Lama |
| | VQVGASLRLSC | | PGEEREFV | M | NTAYLQMNNLEPGDTAV | Y | LVTVSS | |
| | RASG | | AVIS | | YYCAA | | | |
| 4bfb_E | QVQLQESGGG | FTFSSAI | MTWVRQAP | SDGSI | TTYADSVKGRFTISRDNA | A | GRRGPGT | Lama |
| | LVQPGGSLRLS | | GKGREWVS | | RNTLYLQMNSLKPEDTAV | | QVTVSS | |
| | CAASG | | TIG | | YYCTS | | | |
| ATN23916.1 | QVKLQQSGGG | AFFRAGP | MGWYRRAP | RNGR | TIYAPSLKDRFTISRDDDN | NVRTAVAGRN | DYWGQG | Lama |
| | SVQEGGSLRLS | | GNERELVA | | NILYLQMSDLTPGDTAVY | | TQVTVSS | |
| | CASSG | | GIS | | YCNL | | | |
| ATN23921.1 | KVKLQESGGGL | HQLYRPP | VTWYRRAP | GGGT | TIYRDAAKGRFTISRDDV | GKSAYGGY | NYYGQGT | Lama |
| | VRAGESLTLAC | | GKQRELLAF | | KNQVYLQMTNLNPEDTG | | QVTVSS | |
| | AASG | | IT | | VYYCGA | | | |
| ATN23926.1 | QVKLQESGGG | ITFSMYA | MVWHRQAP | SGGS | TRYADSVKDRFTISRDNA | LLGNI | PHWGQG | Lama |
| | LVQAGGSLRLS | | GKQRELVA | | GSTLTLQMNSLKPEDTAV | | TQVTVSS | |
| | CAASG | | GIS | | YYCHA | | | |
| ATN23930.1 | EVKLQESGGG | IAVTNYL | VNWYRRAP | SGGG | RYYTDIAKDRFTISRVVAN | RGFPVVQGALS | EYWGQGT | Lama |
| | SVQAGGSLRLS | | GKPRELVA | | NTVLLHMNSVKPEDTAVY | | QVTVSS | |
| | CTASG | | SIS | | ICRL | | | |
| ATN23928.1 | KVQLQESGGG | ITFGRNI | RRWYRQGS | SWGT | TAYAASVKGRFTISRDNG | VSPRYPYT | TYWGQGT | Lama |
| | LVQAGGSLRLS | | GKQRELVA | | ENTVYLQMNSLKPEDTAV | | QVTISA | |
| | CAASG | | SID | | YTCNA | | | |
| ATN23929.1 | KVQLQQSGGG | SISDFAA | MAWYRQAP | SAGA | LLYAEPVKGRFTISRDNA | YAEA | IYWGQGT | Lama |
| | SVTPGGSLRLS | | GKERDWVG | | KNTVYLQMNSLKPEDTAV | | QVTVSS | |
| | CAASG | | TIF | | YYCRL | | | |
| ATN23931.1 | KVQLQESGGG | IIQSINA | MSWYRRAP | SGGT | AAYADSVKDRFTISRDVL | YDDGT | VYWGQGT | Lama |
| | LVQSGGSLTLS | | GKQREFVAI | | KNTMWMQMDSLKPEDS | | AVTVSS | |
| | CAATG | | IS | | GSYYCRV | | | |
| 4w2o_G | KVQLQESGGG | GTFSINT | LGWYRRAP | SGGI | TRYADSVKGRFTISRDNG | RNWGGGLD | VYWGQGT | Lama |
| | LVQAGGSLRLS | | GKEREFVA | | KNTVYLDMNSLKPEDTAV | | QVTVSS | |
| | CAASG | | RIS | | YYCMY | | | |
| 4w2q_G | KVQLQESGGG | FTFRSSA | MGWYRRAP | TTGT | ADYGDFVKGRFTISRDNA | DPYGMESL | RYWGQG | Lama |
| | LVQVGGSLRLS | | GKQRELVA | | ENTVDLHMNSLKPEDTAV | | TQVTVSS | |
| | CKASG | | SLT | | YYCHE | | | |
| ATN23973.1 | QVQLQESGGG | FTLAEYG | VGWFRQAP | VGGT | THSIDSVKGRFTISRDNM | YSSAYDR | NYWGQG | Lama |
| | LVQPGGSLRLS | | GKERELVAL | | KNTVYLQMNSLNVEDTA | | TQVTVSS | |
| | CTASG | | IA | | VYYCNA | | | |
| CAH18017.1 | LQESGGRLVEA | GTFSWYA | MGWFRQAP | RGGGS | SYYADSVKGRFTISRDNA | GRGAPSDTGRPD | DYWGQG | Lama |
| | GGSLRLSCAVS | | GKEREFVA | | KNTVYLQMNSLKPEDTAV | EY | TQVT | |
| | G | | TIS | | YYCAA | | | |
| CAH18020.1 | LQESGGRLVEA | GTFSWYA | MGWFRQAP | RGGGS | SYYADSVKGRFTISRDNA | GRGSPSDTGRPD | DYWGQG | Lama |
| | GGSLRLSCLVS | | GKEREFVA | | KNTVYLQMNSLKPEDTAV | DY | TQVT | |
| | G | | TVS | | YYCAA | | | |
| CAH18014.1 | LQESGGRLVEA | GTFSWYA | MGWFRQAP | RGGGS | SYYADSVKGRFTISRDNA | GRGAPSDTGRPD | DYWGQG | Lama |
| | GGSLRLSCLVS | | GKEREFVA | | KNTVYLQMNSLKPEDTAV | DY | TQVT | |
| | G | | TVS | | YYCAA | | | |
| CAH18844.1 | LQESGGGLVQ | TIFSIND | MGWYRQA | SDGT | SAYEDSVKGRFTISRDDA | LRASTAGWEPRF | GSWGQG | Lama |
| | PGGSLRLSCVA | ISINH | PGKERELV | | KNMVYLQMNRLKPEDTA | | TQVT | |
| | SG | | AAIT | | VYYCNG | | | |
| CAH18845.1 | LQESGGGLVQ | ITFSDYA | MAWFRQAP | GS | TDYSDSVKGRFTISRDNA | DYGWARPSRHSV | DYWGQG | Lama |
| | AGGSLRLSCTT | | GKGREFVA | | KNTVYLQMNSLRPEDTA | RPEMY | TQVT | |
| | SG | | RSG | | VYYCAG | | | |
| CAH18846.1 | LQESGGGLVPP | RTFSDYS | MAWFRQAP | WGGG | PYYGDSVKGRFTISRDNA | TLDWGTIARMSR | DYWGQG | Lama |
| | GGSLRLSCAAS | | GKDSEFVG | | KNRMYLQMNSLKPEDTA | AGMY | TQVT | |
| | E | | VIS | | TYYCAA | | | |
| CAJ55284.1 | LQESGGGLVQ | RTFSSYA | MGWFRQAP | WSGGS | TYYADSVKGRFTISRDNA | GFALPPS | DYWGQG | Lama |
| | AGGSLRLSCAA | | GKEREFVA | | KNTVYLQMNSLKPEDTAV | | TRVT | |
| | SG | | RIN | | YYCTA | | | |
| CAH18850.1 | LQESGGGLVQ | SIFSPND | MGWYRQA | YSGH | TTYADSVKGRFTISRDTT | FSKWSS | DYWGQG | Lama |
| | PGGSLRLSCAA | | PGLLRELVA | | KKTMYLEMNSLKPEDSG | | TQVT | |
| | SG | | RID | | VYYCGL | | | |
| CAH18851.1 | LQESGGGLVQ | RTGNINT | MTWFRQAP | WGGW | TDYADSVKDRFTISRDGA | DSKYYTVYRSRV | EYWGQGT | Lama |
| | AGDSLRLSCAA | | GKEREFVA | SGGT | KNTMYLQMNSLKFEDTA | TY | QVT | |
| | SG | | GVT | | VYYCAA | | | |
| CAH18852.1 | LQESGGGLVQ | ITFSDYA | MAWFRQAP | GS | TDYADSVKGRFTISRDNA | DLGWARVRRLITR | DYWGQG | Lama |
| | AGGSLRLSCVA | | GKEREFVA | | KSTVYLQMNSLRPEDTAV | PEMY | TQVT | |
| | SG | | RSG | | YYCAG | | | |
| CAH18853.1 | LQESGGGLVQ | RTFSDYA | MGWFRQAP | GS | TYYADSVKGRFTISRDNA | SSWWATAGTGYR | DYWGQG | Lama |
| | AGGSLGLSCAA | | GKEREFVS | | KNTVYLQMNRLKPEDTG | APSWY | TQVT | |
| | SG | | GIG | | VYYCAS | | | |
| CAH18855.1 | LQESGGGLVQ | ITFSDYA | MAWFRQAP | GS | TDYADSVKGRFTISRDNA | DAGWIRSPRHIAR | DYWGQG | Lama |
| | AGGSLRLSCAA | | GKEREFVA | | KNTVYLQMNSLRPEDTA | PEMY | TQVT | |
| | SG | | RSG | | VYYCAG | | | |
| CAF32440.1 | LQESGGGLVQ | TVFSIND | LGWYRQAP | ADGT | SAYEDSVKGRFIISRDDA | LRASNAGWEPRF | GTWGQG | Lama |
| | PGGSLRLSCVA | ISINH | GKERELVA | | KKMVYLQMNSLKPEDTA | | TQVT | |
| | SG | | AIT | | VYYCNG | | | |
| CAH18858.1 | LQESGGSLVQP | SSVSINH | MGWYRQA | SDGE | SAYEDSVKGRFAISRDNL | LRATSAGWELRF | GSWGQG | Lama |
| | GGSLRLSCVAS | | PGKERELV | | KNMVYLQMNTLKAEDTA | | TQVT | |
| | G | | AAIT | | MYYCNY | | | |
| CAJ55287.1 | LQESGGGLVQ | RSVDNYA | MGWFRQAP | WWSG | TEYADSVKGRFTISRDNA | ERGTYFAGRSQD | DDWGRG | Lama |
| | AGDALTLSCAA | | GKELEFVAA | SR | NGMVYLRMSSLRPEDTA | EY | TQVT | |
| | SG | | IS | | VYYCAA | | | |
| CAH18862.1 | LQESGGGLVQ | ITFSLYT | MGWFRQAP | WTGSS | TYYADSVKDRFTTSRDIA | GYRIDTQPMDRD | YYWGQGT | Lama |
| | AGDSLRLSCQA | | GKEREFVS | | KKTVYLQMNSLKPEDTAV | FY | QVT | |
| | SG | | AIT | | YYCAA | | | |
| CAF32446.1 | LQQSGGGLVQ | RAFGYYY | MGWFRQAP | WYDGS | TSYPNSVKGRFTISRDNA | DRSLTVVASSW | RYWGQG | Lama |
| | AGGSLRLSCAA | | GKEREFVA | | KNTVDLQMNSLKSEDTA | | TQVT | |
| | SG | | AIS | | VYYCAG | | | |
| CAH60923.1 | LQESGGGLVQ | RASSSYA | MGWFRQAP | WSGGF | TYYADSVKGRFTISRDNA | RDYRLWSPYQYE | DYWGQG | Lama |
| | AGGSLRLSCAA | | GKEREFVA | | KNTVYLQMNSLKPEETAV | Y | TQVT | |
| | SG | | AIS | | YYCVA | | | |
| CAH60897.1 | LQESGGGLVQ | SSFSIRT | MGWFRQAP | WSGGN | TYYTDFVKGRFTISRDNA | GFAYRSVTYSTR | EYWGQGT | Lama |
| | AGGSLRLSCAA | | GKEREFVA | | KNTVYLQMNSLNPEDTAI | PSFY | QVT | |
| | SG | | AIS | | YYCAA | | | |
| CAH60898.1 | LQESGGGLVQ | LTFSNYV | TGWLRQAP | RSGIS | TDYADSVKGRFTISRDNA | GGGGTYYRRNYK | DYWGQG | Lama |
| | AGGSLSISCAT | | GKDREFVA | | KNTVYLQMNSLKPEDTAV | Y | TQVT | |
| | SG | | SIS | | YYCAA | | | |
| CAH60899.1 | LQESGGGLVQ | LIFSNYV | TGWFRQAP | RSGVS | TDYTDSVKGRFTISRDNA | GGGGTYYQQNYK | DYWGQG | Lama |
| | AGDSLSISCAT | | GKDREFVA | | KNTVYLQMNSLKPEDTAV | Y | TQVT | |
| | SG | | SIS | | YICAA | | | |
| CAH60900.1 | LQESGGGLVQ | RDFSTYA | LAWFRQAP | WTGGS | TYYADSVEGRFTISRDNA | DLPPYGSSWYPP | DYWGQG | Lama |
| | AGGSLRLSCAA | | GKEREFVA | | KNTVYLQMNSLKPEDTAV | RY | TQVT | |
| | SG | | AIT | | YYCAA | | | |
| CAH60902.1 | LQESGGRLVQ | RAFSDYA | VGWFRQAP | WSGGY | TYYADSVKGRFTISRDNA | RHYRLWSPYQYD | DYWGQG | Lama |
| | AGGSLSLSCAA | | GKEREFVA | | KNTAYLQMNSLKPEDTAI | Y | TQVT | |
| | SG | | AIN | | YYCVA | | | |
| CAH60914.1 | LQQSGGGLVQ | RASSSYA | MGWFRQAP | WSGGY | TYYADSVKGRFTISRDNA | RDYRLWSPYQYD | DYWGQG | Lama |
| | AGGSLSLSCAT | | GEEREFVA | | KNTVYLQMNSLKPEDTAV | Y | TQVT | |
| | SG | | AIS | | YYCVA | | | |
| CAH60922.1 | LQESGGGLVQ | SSFSIRT | MGWFRQAP | WSGGN | TYYTDFVKGRFTISRDNA | GFAYRSVTYSSR | EYWGQGT | Lama |
| | AGGSLRLSCAA | | GKEREFVA | | KNTVYLQMNSLKPEDTAI | PSFY | QVT | |
| | SG | | AIS | | YYCAA | | | |
| CAH60939.1 | LQQSGGGSVQ | STFVINP | MRWYRQAP | FNGA | TNYADSVKGRFTISNNNA | VVWVGSESY | DYWGQG | Lama |
| | AGGSLRLSCAA | | GKQRELVA | | KNTVFLQMNSLKPEDTAV | | TQVT | |
| | SA | | GIT | | YYCNV | | | |
| CAH60956.1 | LQESGGGLVQ | GTFSIYT | MGWFRQAP | RSGIS | TDYGDSVKGRFTISGDNA | DKTTYWNIPRDEY | DFWGQGT | Lama |
| | AGGSLRLSCAA | | GKEREFVA | WKGAM | KNTVFLQMNSLKPEDTA | | QVT | |
| | SG | | AIT | | MYYCAA | | | |
| CAH60927.1 | LQESGGDLVQ | STLSIKA | MAWYRQAP | SRGS | PKYADSVKGRFTISRDNA | RTTFLTEGWR | NYWGHGT | Lama |
| | PGGSLRLSCAA | | GKQREMVA | | KNTVSLQMNSLKPEDTAV | | QVT | |
| | SG | | AIT | | YYCNA | | | |
| CAH60928.1 | LQESGGGLVQ | SFGYIGG | MGWYRQTP | SDLS | ADYADSVKGRFTISRDNA | RRPSSSTIL | DDWGQG | Lama |
| | PGGSLRLSCAA | | GKQREFVA | | RHTVYLEMNNLKPEDTG | | TQVT | |
| | SG | | WIT | | VYYCNA | | | |
| CAH60957.1 | LQQSGGGLVQ | GTFSIYT | MGWFRQAP | RSADS | TDYADSVKGRFTISRDNA | DKTTFWNIPRDEY | DYWGQG | Lama |
| | AGNSLTLSCAA | | GKEREFVA | WNGA | KNTVYLQMNSLKPEDTAV | | TQVT | |
| | SG | | AIS | M | YYCTA | | | |
| CAH60933.1 | LQESGGGLVQ | GTSSIYT | MGWFRQAP | RSAIS | TDYADSVKGRFTISTDNA | DRTTSWNIPRDEY | DYWGQG | Lama |
| | AGGSLRLSCAA | | GKEREFVA | WKGAS | KNTVYLQMNSLKPEDTAI | | TQVT | |
| | SG | | AIS | | YFCVA | | | |
| CAH60934.1 | LQESGGGLVQ | INSRIYR | TGWYRQAP | DGGS | TNYGDSVKGRFTISRDNA | NTVANLGR | NYCGQGT | Lama |
| | PGGSLRLSCVA | | GNQRELVA | | KNTVYLQMNNLKPEDTA | | QVT | |
| | SG | | RIS | | VYQCCV | | | |
| CAH60935.1 | LQQSGGGLVQ | NTFVINP | MGWYRQA | FTGT | TNYADSVKGRFTASRDN | VVWVGPKSY | DSWGQG | Lama |
| | AGGSLRLSCTA | | PGKQRELV | | TKNTVFLQMNSLKPEDTA | | TQVT | |
| | AG | | AEVT | | VYYCNA | | | |
| CAH60937.1 | LQESGGGLVQ | TIFSLHA | MGWYRQA | RAGT | TDYGDSVKGRFTISRDNA | RTIVPVPK | NYWGQG | Lama |
| | PGGSLRLSCAS | | PGKQRELV | | ENTVYLQMGSLKPDDTA | | TQVT | |
| | SR | | ATIT | | VYYCNA | | | |
| CAH60938.1 | LQESGGGLVQ | RAFT | VAWFRQAP | WIGSS | TLYADSVKGRFTISRDNA | TRALAIATTSEKY | DYWGQG | Lama |
| | AGDSLRLSCVA | | GKEREFVS | | KNTVYLQMNSLKPEDTAV | | TQVT | |
| | SG | | GIR | | YYCAA | | | |
| CAH60940.1 | LQESGGGLVQ | NTFVLNP | MRWYRQAP | FIGT | TNYADSVKGRFTISTDNA | VVWVGSKSY | DSWGQG | Lama |
| | AGGSLRLSCAA | | GKQRELVA | | KKTVFLQMNSLKPEDTAV | | TQVT | |
| | SA | | GVT | | YYCNV | | | |
| CAH60941.1 | LQESGGGLVQ | TTVSSNT | MDWHRQA | RGDV | LTYSDSVKGRFIISRDPTK | GLNIGTGIIPH | KYWGQGT | Lama |
| | AGGSLRLACAA | | PGKKRELV | | NMVYLQMNELKPEDTAV | | QVT | |
| | SG | | ARIS | | YYCAA | | | |
| CAH60942.1 | LQESGGGLVQ | IIFRFNA | MGWYRQE | SGGTT | INYADSVKGRFTISRDNT | YPGFQLTTP | KWWGQG | Lama |
| | PGGSLRLSCAA | | PGKQREFV | | QKTVYLQMNSLKYDDTA | | TQVT | |
| | SG | | AGIS | | VYFCAA | | | |
| CAJ34301.1 | QLQASGGGLV | LTFSNYV | MGWFRQAP | WSGW | KTDSNSAKGRFTISRNNA | AAAKTRLPQIVYR | GIWGQGT | Lama |
| | QAGGSLRLSCA | | GKEREFVA | | KNTVDLQMNSLKPEDTA | TTGDY | QVTVSS | |
| | ASG | | GIS | | VYYCAA | | | |
| CAJ34302.1 | QLQASGGGLV | LTFSNYV | MGWFRQAP | WSGW | KTDSNSAKGRFTISRNNA | AAARTRLPQIVYR | GIWGQGT | Lama |
| | QAGGSLRLSCA | | GKEREFVA | | KNTVDLQMNSLKPEDTA | TTGDY | QVTVSS | |
| | ASG | | GIS | | VYYCAA | | | |
| CAJ55317.1 | LQQSGGGLVQ | RAFQYYY | MGWFRQAP | WYDGS | TSYADSVKGRFTISRDNA | DRGLTAVASSW | RYWGQG | Lama |
| | AGGSLRLSCAA | | GKEREFVA | | KNTVDLQMNSLKSEDTA | | TQVT | |
| | SG | | AIS | | VYYCAG | | | |
| CAJ55264.1 | LQESGGGLVQ | LIFNNHY | LGWFRQAP | WYDGS | TSYADSVKGRFTISRDSA | DRGLTASPSSW | RYWGQG | Lama |
| | AGGSLRLSCAA | | GKEREFVA | | KNTVDLQMNSLKSEDTA | | TQVT | |
| | SR | | AIS | | VYYCAG | | | |
| CAJ55266.1 | LQESGGGLVQ | LTFSSYY | MGWFRQTP | WYDDS | TSYADSVKNRFTISRDNA | GRSLTVVASSW | RYWGQG | Lama |
| | AGGSLRLSCAA | | GKEREFVA | | KNTVYLQMSSLKPEDTAV | | TQVT | |
| | SG | | AIG | | YYCAG | | | |
| CAJ55279.1 | LQQSGGGMVQ | LIFNNHY | LAWFRQAP | WYDGS | TSYTNSVKGRFTISRDNA | DRGLTSVASSW | RYWGQG | Lama |
| | AGGSLRLSCAA | | GKERMFVA | | KNTVDLQMNSLKSEDTA | | TQVT | |
| | SQ | | AIS | | VYYCAG | | | |
| CAJ55270.1 | LQESGGGLVQ | LTFSRYY | LAWFRQAP | WYDYS | TYYADSVKGRFTISRDNA | GRSLTDVPGSW | SYWGQGT | Lama |
| | AGGSLRLSCAA | | GKERELVAT | | KNTMYLQVNSLKPEDTAV | | QVT | |
| | SG | | IG | | YYCAG | | | |
| CAJ55280.1 | LQQSGGGLVQ | RAFNYYY | MGWFRQAP | WYDAS | TSYADSVKGRFTISRDNA | DRGLTDVPSSW | RYWGQG | Lama |
| | AGGSLRLSCAA | | GKEREFVA | | KNTVDLQMNSLKSEDTA | | TQVT | |
| | SG | | AIS | | VYYCVG | | | |
| CAJ55281.1 | LQQSGGGLVQ | LSFNHYY | MGWFRQAP | WYDHS | TSYADSVKGRFTISRDNA | DRGLTAVDSSW | RYWGQG | Lama |
| | AGGSLRLSCQA | | GKNREFVA | | KNTVDLQMNALKSEDTA | | TQVT | |
| | SR | | AIS | | VYYCVG | | | |
| CAJ55285.1 | LQESGGGLVQ | RTFSSNA | MGWFRQAP | FGGHS | TNYRDSVEGRFTISRDDV | STGSQATMNLRS | TYWGQGT | Lama |
| | AGGSLRLSCAA | | GKEREFVG | | KNTVYLQMNSLKPEDTAV | Y | QVT | |
| | SG | | AIS | | YYCAA | | | |
| CAJ55286.1 | LQESGGGLVQ | RTVSSKA | MGWFRQAP | WGGHS | ANYRDSVEGRFTISRDDS | STGSQATMSFRS | GSWGQG | Lama |
| | AGGSLRLSCAA | | GKEREFVG | | KNTVYLQMNSLKPEDTAV | Y | TQVT | |
| | SG | | SIS | | YYCAA | | | |
| CAJ55289.1 | LQQSGGGLVQ | RTLSTYP | MGWFRQAP | WRG | SSTDTGDSVKGRFTTFKD | GRSYEARSFSAN | AYWGQGT | Lama |
| | AGASLRLSCTA | | GKEREFVA | | NASLYLQMKGLKPEDSA | DY | QVT | |
| | PG | | AVD | | VYYCAA | | | |
| CAJ55290.1 | LQESGGGLVQ | ITLSFYG | MGWFRQAP | SNGDS | TYYEDSVKGRFTISRDNA | AFRLSGSHLFDRL | VHWGQG | Lama |
| | AGGGLRLSCAA | | GGEREFVA | | KSTVFLQMNRLKPEDTAV | YY | TQVT | |
| | SG | | AIT | | YYCGA | | | |
| CAJ55294.1 | LQQSGGGLVQ | RTFSSYS | VGWFRQAP | WSGSS | TYSADSVKGRFTISRDNA | DRSRAEWLVTNE | DYWGQG | Lama |
| | AGGSLRLTCAA | | GNEREFVA | | KNTAYLQMNSLKPEDTAV | ADY | TQVT | |
| | SG | | AIS | | YYCAA | | | |
| CAJ55295.1 | LQESGGGLVQ | RTFSRYV | MGWFRQAP | GSGSS | TSYADSVKGRFTISRDNA | VLSFPSTKERDY | TYWGQGT | Lama |
| | AGGSLRLSCAA | | GKEREFVA | | KNAVYLQMNSLKPEDTA | | QVT | |
| | SG | | AIV | | VYYCAA | | | |
| CAJ55296.1 | LQQSGGGLVQ | RTVENYA | MGWFRQTP | WWSG | ADYSDSVKGRFTISRDNA | EPGTYFAGRFES | DYWGQG | Lama |
| | AGDPLTLSCAA | | GKEPEFVA | SR | KKTLYLQMSSLQPEDTGV | EY | TLVT | |
| | SG | | AIS | | YYCAA | | | |
| CAJ55299.1 | LQESGGGLVQ | SISRFND | MAWSRQAP | SGGN | TRYADSVKGRFTISRDNA | QLPTGWVRRRGS | DDWGQG | Lama |
| | PGGSLRLSCAA | | GEQREFVA | | KNTVYLQMTSLKVEDTAV | | TRVT | |
| | SG | | RID | | YHCNV | | | |
| CAJ55300.1 | LQQSGGGLVQ | RTFNSYA | MGWFRQAP | WGGG | TSYEDSVKGRFTISRDND | GDSTHHIRALSP | EYWGQGT | Lama |
| | AGGSLRLSCTA | | GKKRAFVA | S | KNTVYLRMNSLKPEDTAV | | QVT | |
| | SG | | AIS | | YYCAQ | | | |
| CAJ55302.1 | LQESGGGLVQ | RTFSR | YGWFRQAP | WSSGS | TYYADSVKGRFTISRDNA | SSLYLALQRAEY | DYWGQG | Lama |
| | AGGSLRLSCAA | | GKEREIVAS | | KNTVYLQMNGLKPEDTA | | TQVT | |
| | SG | | IR | | VYSCAA | | | |
| CAJ55303.1 | LQQSGGGLTQ | RTFGTYA | VGWFRQAP | RSGGS | TYYTDSVKGRFTISRDNA | RRHGEY | DYWGQG | Lama |
| | AGGSLRLSCAA | | GKGREFVG | | KNTVYLQMNGLKPEDTA | | TQVT | |
| | SG | | AIR | | VYYCAA | | | |
| CAJ55304.1 | LQESGGGLVQ | STFSSYV | MGWFRQAP | RRGGR | GVYSDSVKGRFTISRDNA | QRRGIPRY | EYWGQGT | Lama |
| | AGVSLRLSCAA | | GKEREFVA | | KNTLYLQMNSLKPEDTAV | | QVT | |
| | SG | | AIT | | YYCAA | | | |
| CAJ55305.1 | LQQSGGGLVQ | RTFTLEA | MGWFRQAP | WRSGS | MDYADSVKGRFTISRDIA | DRDTLRISGAGGA | DYWGQG | Lama |
| | AGDSLRLSCAA | | GKEREFVA | K | KNTVYLQMNSLKPEDTAV | M | TLVT | |
| | SG | | AID | | YYCAA | | | |
| CAJ55308.1 | LQESGGGLVQ | DFFSINN | MGWYRQA | SRGS | TNYADSVKGRFTISRDNA | DETSGWASLR | HYWGQG | Lama |
| | PGGSLRLSCAA | | PGKQRELV | | KNTVYLQMNSLKPEDTAV | | TQVT | |
| | SG | | ATIS | | YYCNA | | | |
| CAJ55311.1 | LQESGGGLVQ | RTFDNWT | LGWFRQAP | WTGGV | TDYADSVKGRFTISRDKA | RAGWDLNRFDY | RYWGQG | Lama |
| | AGGSLRLSCAA | | GKEREFVA | | KNTVALQMNSLKPENTAV | | TQVT | |
| | SG | | AIT | | YYCAA | | | |
| CAJ55312.1 | LQESGGGLVQ | RTFSSYT | MAWFRQAP | WSDGN | TYYADSVKGRFTISRDNA | DYSGLTTRWGAY | DYWGQG | Lama |
| | AGGSLRLSCAA | | GKEREFVSI | | KNTVYLQMNSLKAEDTAV | | TQVT | |
| | SG | | IS | | YYCAA | | | |
| CAJ55313.1 | LQESGGGLVQ | RTFSGGA | MGWFRQAP | WSGHS | TYYGDSVKGRFTISRNNA | RESAGWSSYR | NYWGQG | Lama |
| | AGGSLRLSCAA | | GKEREFVA | | RNTVYLQMNSLKPEDTA | | TQVT | |
| | SG | | AIS | | VYYCNA | | | |
| CAJ55314.1 | LQESGGGLVQ | ISFSIRD | MGWYRQA | SG | GTTNYVDSVKGRFTISRD | DWTTGWRTE | RSWGQG | Lama |
| | AGGSLRLSCAA | | PGKQRELV | | NAKVYLLMNILKPEDTAV | | TQVT | |
| | SG | | ATIS | | YYCAV | | | |
| CAJ55315.1 | LQESGGGLVQ | RTFSNYN | MGWFRQAP | WSGW | PYYADSVKGRFTISRDNA | DRSLTRSASSY | NYWGQG | Lama |
| | AGDSLRLSCAA | | GKEREFVA | SGGN | KNTVYLQMNSLKPEDTAV | | TQVT | |
| | SG | | VIG | | YYCAA | | | |
| CAJ55316.1 | LQQSGGGLVQ | RTFNSYN | MGWFRQSP | WYDSS | TNYADSLKGRFTISRDNA | GRSYSSNPAAY | NYWGQG | Lama |
| | AGGSLRLSCAT | | GKEREFVAL | | KNTMYLQMDSLKPEDTA | | TQVT | |
| | SG | | TS | | VYYCAA | | | |
| CAJ55319.1 | LQESGGGLVQ | SIRTFNT | MGWYRQG | NSGS | TNYADSVKGRFTISRDNA | DVQGGGWGSIQ | RYWGQG | Lama |
| | AGGSLRLSCAA | | TGNERELIA | | INTVYLQMNSLKPEDTAV | | T | |
| | SG | | SIT | | YYCNI | | | |
| CAJ55320.1 | LQESGGGLVQ | RTWNPSA | MGWFRQAP | RGGGS | VYYADSVKGRFTISREVD | QSPGMSGTYSRS | PYWGQGT | Lama |
| | AGGSLRLSCAT | | GKGREFVA | | KNAVYLQMNSLKSEDTA | DVY | QVT | |
| | SG | | AVS | | VYYCAA | | | |
| CAJ57593.2 | LQQSGGGLVQ | RTFSSSL | MGWFRQAP | WNGSV | ISYTDSVKGRFTISRDNAK | AALEYCTGTGCR | GYWGQG | Lama |
| | AGGSLRLSCAA | | GKEREFVA | | NTVWLQMNSLKPEDTAV | Y | TQVT | |
| | SG | | DID | | YYCAQ | | | |
| CAL18273.1 | QVQLQESGGG | RTGRNYD | MGWFRQAP | WNGG | TVYADSVKGRFTISRDNG | TSVRGLVASVRR | DYWGQG | Lama |
| | LVQAGGSLRLS | | GREREFVA | N | KNMMYLQMSSLKPEDTA | YEY | TQVTVSS | |
| | CAASR | | AIT | | VYYCAG | | | |
| 3qxw_E | QVQLVESGGG | RSSRSWA | MAWFRQAP | GDGRL | TTYGDSVKGRFTISRDNA | DDNYVTASWRSG | DYWGQG | Lama |
| | LVQAGGSLRLS | | GKEREFVA | | EYLVYLQMDSLKPEDTAV | P | TQVTVSS | |
| | CAASR | | KIS | | YYCAA | | | |
| CBX21160.1 | LQQSGGGSVQ | LAFSDYA | MGWFRQAP | QIGNN | PNYEDSVNGRFTIARDNA | DRSSLQLYASRV | DYWGQG | Lama |
| | AGGSLRLSCAV | | GKEREFVA | | KNTMYLQMNSLKPEDTA | GDEY | TQVTVSS | |
| | SG | | AIS | | VYYCAA | | | |
| CBX21183.1 | LQESGGGLVQ | FTFSSYA | MAWFRQAP | RSSGR | TYHADSVKGRFTVSRDN | SYFWSDTPFALN | RYWGQG | Lama |
| | PGGSLRLSCAA | | GKERVFVA | | AKNTAYLQMNSLSPEDTA | Y | TQVTVSS | |
| | SG | | ALD | | VYYCAA | | | |
| CBX21184.1 | LQESGGGLVQ | RTSSAFA | IGWFRQAP | WSTGR | TSSADSVRDRFTISRDTP | DPNGRGY | YYWGQG | Lama |
| | AGGSLRLSCAA | | GKEREFVG | | KNLVYLQMDSLKPEDTAV | | SQVTVSS | |
| | SG | | SII | | YYCAA | | | |
| CBX21185.1 | LQESGGGLVQ | RTFPIYG | LGWFRQAP | WSTGR | TYNADSVEGRFAISRDNA | DRDGRGY | VYWGQGT | Lama |
| | VGGSLRLSCAA | | GKEREYVA | | KNTVYLQMNSLRPEDAA | | QVTVSS | |
| | SG | | SIT | | VYYCAG | | | |
| CBX21180.1 | LQESGGGSVQ | LAFSNYH | MGWFRQAP | QIGNN | PNYEDSVNGRFTIARDNA | DRSSPSLRLVASR | DYWGQG | Lama |
| | AGGSLRLSCAV | | GKEREFVA | | KNTMYLQMNSLKPEDTA | VGDEY | TQVTVSS | |
| | SG | | AIS | | VYYCAA | | | |
| CBX21177.1 | LQESGGGLVQ | RTFTSYD | MGWFRQAP | WSGET | TYYADSVKGRFTISRDNT | HYGSRWKSVSVE | TYWGRGT | Lama |
| | AGGSLRLSCAS | | GKERELVA | | KDTVYLQTNNLKPEDTAV | NY | QV | |
| | SA | | AIN | | YYCAA | | | |
| CBX21165.1 | LQESGGGSVQ | LAFSNYH | MGWFRQAP | QIGNN | PNYEDSVNGRFTIARDNA | DRSSPSLRLVASR | DYWGQG | Lama |
| | AGGSLRLSCAV | | GKEREFVA | | KNTMYLQMNSLEPEDTA | VADEY | TQVTVSS | |
| | SG | | AIS | | VYYCAA | | | |
| CBX21166.1 | LQESGGGSVQ | LAFSNYH | MGWFRQAP | QIGNN | PNYEDSVNGRFTIARDNA | DRSNPSLRLVAS | DYWGQG | Lama |
| | AGGSLRLSCAV | | GKEREFVA | | KNTMYLQMNSLKPEDTA | RVGDEY | TQVTVSS | |
| | SG | | AIS | | VYYCAA | | | |
| CBX21168.1 | LQESGGGLVQ | RTASINA | IAWYRQAP | SVGR | SNYADAVEGRFTISRDND | AVGSRWY | ELWGQGT | Lama |
| | AGGSLRLSCAA | | GKEREFLA | | KNTVYLQMNSLKPEDTAA | | QVTVSS | |
| | SG | | RIN | | YYCAA | | | |
| CBX21169.1 | LQQSGGGLVQ | FTFSTYA | MGWFRQAP | RSSDS | TYYVDSVKGRFTVSRDN | SYFWSNTPFALN | RYWGQG | Lama |
| | PGGSLRLSCAA | | GKGREFVA | | AKNTAYLQMNSLSPEDTA | Y | TQVTVSS | |
| | SG | | ALD | | VYYCAA | | | |
| CBX21173.1 | LQQSGGGLVQ | STFSRYR | MGWFRQAP | WSGGS | TYYEDSVKGRFTISRDNA | SQWDGRTWTRP | EYWGQGT | Lama |
| | AGGSLSLSCAF | | GKEREFVG | | KNTVYLQMDSLASEDTG | HAY | QVTVSS | |
| | SG | | TIN | | VYYCAA | | | |
| CBX21176.1 | LQESGGGLVQ | QSFSGYA | MGWFRQAP | WSGNS | TDYGDFVKGRFTVSRDN | DLGAYGGLGLSY | DYWGQG | Lama |
| | TGGSLRLSCAL | | GKEQEFVA | | AKNTVYLQMNSMKPEDT | | TQVTVSS | |
| | SD | | AIN | | AVYYCAA | | | |
| CBX21179.1 | LQQSGGGLVQ | RTFTSYT | MAWFRQAP | GNGGE | TYVPDSVKGRFTISRDNA | EILYCSGYGCRDP | NYWGQG | Lama |
| | AGGSLRLSCAA | | GKEREFVG | | KNTVYLELNSLNPEDTAV | RLY | TQVTVSS | |
| | SG | | VVI | | YTCAA | | | |
| CBX21181.1 | LQQSGGGLVQ | GTFNPYA | MAWYRQAP | RSGDI | TYYADSVKGRFTISRDSA | STLLPTN | DYWGQG | Lama |
| | AGASLRLSCAT | | GREREFVA | | RNTIYLQMNSLKPEDTAV | | TQVTVSA | |
| | SE | | RIY | | YYCSA | | | |
| CBX21186.1 | LQESGGGLVQ | RTFRNYA | MGWFRQAP | WSGDN | TYYADSVKGRFSISMDNA | GFITGIHPYEY | HYWGQG | Lama |
| | AGGSLRLSCAA | | GKERDLVA | | KNTVLLQMNSLKPEDTAV | | TQVTVSS | |
| | SG | | RIE | | YYCAA | | | |
| 035502 | GGLVQSGGSL | FIFVRNP | MSWYRQSP | GASN | KEYADFAKGRFTISRENV | TINGR | DYWGQG | Lama |
| | TLSCEASG | | GKGREFVA | | KRTAYLIMTGLKPEDAAD | | TQVTVSS | |
| | | | AID | | YFCSI | | | |
| 035499 | GGDVQTGGSL | FSLTSST | IGWFRQAP | REKEE | DRYMNSVAGRFTISKDKT | GWTWDDCLHQE | HYWGQG | Lama |
| | RLSCSVPR | | GSGREAVA | | KNMMYLNMTGLKPEDTA | TY | TQVTVSS | |
| | | | CMT | | LYVCAV | | | |
| 035504 | GGLVQTGGSL | TLHSISD | MAWYRQPP | TDGS | TDYDDSVRGRFTITGNNA | VIVSVIRQWP | GNWGQG | Lama |
| | RLSCAASK | | GKRRKGVA | | ENTISLQMNNLRPEDSAV | | AQVTVIS | |
| | | | TIS | | YFCQA | | | |
| 035506 | GGLVQAGDSL | DISAFTT | MGYYRQFP | RAGF | ETYADAVKGRFTVFRDTS | PYIGR | AYWGRGT | Lama |
| | SPSCRPSM | | GQLRELVAT | | KNTVDLQMNDLKVEDTA | | QVTVSS | |
| | | | IT | | VYYCNA | | | |
| 035507 | GGSVQTGGSL | ISAVNQ | VRWFRRPP | SGGV | TKYGDSVKGRFVISHDNA | RIDWTPHEY | EVWGQGI | Lama |
| | RVSCAASG | | GMEREFVA | | DNSVVHLQMNSLKIEDTG | | EVTVSS | |
| | | | RIN | | VYYCN | | | |
| 035510 | GGSVQAGESL | NIFGLNA | VGWYRQAP | SDSN | TIYADSVKGRFTISRNNAK | VQRGRYGYEGDR | NYWGQG | Lama |
| | RLSCVASG | | GSQRELVA | | NTVYLQMNSLKPEDTAVY | | TQVTVSS | |
| | | | RIN | | YCNV | | | |
| 035512 | GGSVQAGGSL | DTYSSQC | MGWFRQAP | SGSGS | TAYADSVKGRFTISRDNA | DSESYGCGYSIVP | DYRGQGT | Lama |
| | RLSCAASG | | GKQREGVA | | KNTVYLQMNSLIPEDTAIY | GF | QVTVSS | |
| | | | VIN | | YCAA | | | |
| 035513 | GGSVKAGGSL | FADSIRC | LAWFRQAS | TGRHN | TYYDDDVKGRVTVSLDST | VYGYYNKPCARA | EYWGQGT | Lama |
| | TLSCKVSA | | GKDRDFIAA | | GAIVSLQVNTLEPADSGM | SSEY | QVTVSS | |
| | | | LH | | YYCAF | | | |
| 035514 | GGSVQAGGSL | DTPSLKC | MAWFRQAP | PVDNS | ITYADSVKGRFTISRDNAK | TRGTDPDCSKRL | GFWGQG | Lama |
| | KLSCLGSG | | GKGREGVA | | TTLSLQMNGLKPDDTAM | SAPRFF | TQVTVSS | |
| | | | VIA | | YYCAA | | | |
| 035515 | GGSVEAGGSL | YIYSGKC | LGWFRQAP | DARI | TYYDDSVKGRFTISVDRV | EGPQLLSSRLCL | SHWGQG | Lama |
| | NISCVVSE | | GEERERVA | | KNTVNLQMNSLKPEDTG | | TWITVSS | |
| | | | TIY | | MYYCAS | | | |
| 035517 | GGSGQAGGSL | YKYDNYC | VGWFRQAP | RSGGS | TIYAESVKGRFAISQDNTK | GSGDCEMIETAL | DAWGQG | Lama |
| | RLSCHVSG | | GKEREWVA | | GTVHLQMNNLEPEDTAK | | TTVTVSS | |
| | | | LIE | | YYCAA | | | |
| 035520 | GGSVQAGGSL | FIHNYNKY | MGWFRQSP | GFGRS | VYYHDDVKGRFTIYRDNA | ASLDVTASLYYMN | SDWGQG | Lama |
| | SLSCAASG | S | GKEREGVA | | KKMVFLQMNSLQPNDTAI | PAWW | TQVTVSS | |
| | | | AID | | YYCAS | | | |
| 035521 | GGSVQAGGSL | DTYSDCN | MAWYRQVT | VDGT | IHFEDNVKGRFTISQDDA | ECRVQVGYGPIQ | NYWGQG | Lama |
| | RLACAASG | | GKERELVS | | KKMLYLQMSNLSPEDTA | DY | TQVTVSS | |
| | | | VIL | | MYYCNT | | | |
| 035522 | GGSVQAGGTL | LTFSTYC | MGWFRQAP | AGGGS | TYYADSVKGRFTIFQDNA | DLDDCNEGGYIVT | SYWGQGT | Lama |
| | RLSCVGSG | | GKEREGVA | | KNTMYLQMNSLKPEDTAI | MS | QVTVSS | |
| | | | VIN | | YYCAA | | | |
| 035523 | GGSVQAGGSL | TTECYYD | MSWYCQAP | SDEG | SRYADSAKGRFTISQDNA | VVKWCPRRLTGL | TYWGQGT | Lama |
| | RLSCVHSG | | GKEREFVS | | KNTVLLQMNNLKPEDTA | | RVTVSS | |
| | | | GID | | MYYCRL | | | |
| 035525 | GGSVQAGGSL | EYTLKVHY | VAWFCQAP | TLDDR | TMYADSVKGRFAISRDDA | DFQGGHLLDARV | TYWGQGT | Lama |
| | RLSCTTTS | | GKEREGVA | | KNTVYLEMNTLKFEDTAM | Y | QVTVSS | |
| | | | AIY | | YYCAA | | | |
| 035528 | GGSVQAGGSL | IDYRPTY | VGWFRQAP | IGAES | GSYSKDVKDRFTVSQDT | TPSYSRNWRNY | SHWGQG | Lama |
| | RLSCSAST | | GKEREWVA | | AKRTVYLQMTSLNPEDTA | | TQVTVSS | |
| | | | GVY | | AYYCGS | | | |
| 035529 | GGSVQAGGSL | HTWNSYV | MGWFRQTP | IGFGS | TFYADSVKGRFTIREQRG | VYNQLWLVQLRA | THWGQGI | Lama |
| | TLSCTASG | | GKEREGVA | | MNTVHLQMNNLKPEDTG | TEY | QVPISS | |
| | | | VIS | | MYYCAA | | | |
| 5ovw_J | QMQLVESGGG | STLDDYA | IGWFRQAP | SSGDS | TNYADSVKGRFTVSRDN | AHRIFGGCLVIHS | VSWGQGT | Lama |
| | LVQPGGSLRLS | | GKEREGVS | | AKNTVYLQMNDLRPEDT | SGY | PVTVSS | |
| | CAAPE | | CIG | | AVYYCAA | | | |
| 5sv4_B | EVQLVESGGGL | RTLGDYG | VAWFRQAP | RSTII | TDYADSVRGRFSISADSA | IANPVYATSRNSD | GHWGQG | Lama |
| | VQAGDSLRLSC | | GKEREFVS | | KNTVYLQMNSLKPEDTAV | DY | TQVTVSS | |
| | TASG | | VIS | | YYCAV | | | |
| 5vaq_B | QVQLVESGGG | RTFSPYV | GGWFRQAP | WSGGT | KLYADSVKGRFTISRDNA | RRINEVLTTAPDY | DFWGQGT | Lama |
| | LVQAGGSLRLS | | GKEREFVA | | KNTVYLQMNTLKREDTAV | | QVTVSS | |
| | CAASQ | | AIS | | YYCAA | | | |
| 5vl2_H | QVQLQQSGGG | RTHTPYA | MGWFRQAP | GVAAT | TTYADSVRGRFTISRDDA | RASMAVSTSPRV | PIWGQGT | Lama |
| | LVQAGGSLRLS | | GKEREFVG | | KATVYLQMNSLKPEDTAV | Y | QVTVSS | |
| | CAASG | | GIG | | YYCAT | | | |
| 5vm0_B | EVQLVESGGGL | RTYTPYA | MAWFRQAP | GIDGT | AAYADSVRGRATISRDSA | RASMQVLTSPRV | PIWGRGT | Lama |
| | VQTGDSLRLSC | | GKEREFVA | | KKTVYLQMNSLKPEDTAV | Y | QVTVSS | |
| | AASG | | GIG | | YSCAT | | | |
| 5vm6_A | QVKLQQSGGG | RALSSTI | VGWFRQIP | WSSSD | TWYADSVKGRFTISKDDA | ALRRPGSDASDY | PYWGQGT | Lama |
| | MVQTGDSLRLS | | GKEREFVG | | ANGVHLQMSSLKPEDTA | TRIPDY | QVTVSS | |
| | CVGSR | | GIA | | VYYCAS | | | |
| 6b20_F | QVQLVESGGG | SIFSINA | MGWYRQA | RGGR | TNYADSVKGRFTLSRDN | GRSR | GYWGQG | Lama |
| | LVQAGGSLRLS | | PGKQRELV | | AKNTVYLQMNSLKPEDTA | | TQVTVSS | |
| | CAASG | | AAIT | | VYYCNV | | | |
| 5c1m_B | QVQLVESGGG | RTSYP | MGWFRRAP | WSGID | PTYADSVADRFTTSRDVA | RAPVGQSSSPYD | DYWGQG | Lama |
| | LVRPGGSLRLS | | GKEREFVA | | NNTLYLQMNSLKHEDTAV | Y | TQVTVSS | |
| | CVDSE | | SIT | | YYCAA | | | |
| 6c9w_B | VQLVESGGGLV | GTFSTFN | MGWFRQDL | WTGGR | AYYGDSVKGRFTISRDNA | QGTNGGGYSEAT | NYWGQG | Lama |
| | QAGDSLRLSCA | | GKEREFVA | | KNTVYLQMNSLKPEDTAV | SY | TQVTVSS | |
| | ASG | | AIR | | YYCAR | | | |
| 6fyt_I | EVQLVESGGGL | SIFDIYA | MDWYRQAP | RDGS | TNYADSVKGRFTISRDNA | SLYRDPLGVAGG | VYWGKGA | Lama |
| | VQPGGSLRLSC | | GKQRDLVA | | KNTLYLQMNSLKPEDTAV | MG | LVTVSS | |
| | AVSI | | TSF | | YLCHV | | | |
| 6ehg_C | QVQLVETGGG | SIFSLNA | MGWFRQAP | RSGGR | TYYADSVKGRFTISRDNG | GTGWSPQTDNEY | NYWGQG | Lama |
| | LVQAGGSLRLS | | GKEREFVA | | KNMVYLQMHSLKPEDTAI | | TQVTVSS | |
| | CAASG | | TIN | | YYCAA | | | |
| 6eqi_B | QVQLVESGGG | RPFSTFD | LAWFRQAP | RGGS | TFYADSVSGRFTISGGST | SITGYGLVDSASH | TIWGQGT | Lama |
| | LVQAGGSLKLS | | GKEREFVS | | KNTLYLQMNSLKPEDTAI | Y | QVTVSS | |
| | CTTSG | | GLR | | YYCAR | | | |
| 5lz0_B | DVQLVESGGG | RTFSSYS | MGWFRQAP | RSDNS | TYYADSVKGRFTISRDSA | TPYGSRYYLRELR | DYWGQG | Lama |
| | LVQAGGSLRVS | | GKEREFVA | | KNTVYLQMNSLKPEDTAV | EY | TQVTVSS | |
| | CAASG | | AIS | | YYCAA | | | |
| 6f0d_B | QVQLVQSGGG | RTFSMYT | MGWFRQAP | GRGLS | PDIADSVNGRFTISRDNA | DLQYGSSWPQRS | DYWGQG | Lama |
| | LVQAGGSLRLS | | GKEREFVA | | KNTLYLQMDSLKPEDTAV | SAEY | TTVTVSS | |
| | CAFSG | | ANR | | YYCAA | | | |
| 6f2w_B | QVQLVESGGG | RTFSSRA | MGWFRQAP | WSGSY | TEYADSVKGRVTISRDNA | KNGGAASNYPND | VYWGQGT | Lama |
| | VVQAGGSLRLS | | GEGREFVA | | KNTVYLQMNSLKPGDTA | Y | QVTVSS | |
| | CAASG | | TIS | | VYHCAA | | | |
| 6f5g_B | QVKLEESGGGL | RTFSAYA | MGWFRQVP | DSDAS | IYYTDSVKGRFTISRDNAK | RQYYSGRVYSTF | DYWGQG | Lama |
| | VQAGGSLRLSC | | GREREFVA | | NTVYLQMNSLIPDDTAVY | REEY | TQVTVSS | |
| | SPSD | | TIR | | YCAA | | | |
| 6fpv_B | QVQLVESGGG | SFFSIND | MGWYRQA | SGGS | TNYADSVEGRSTISSDNA | NVRLREYRTTSY | HYWGQG | Lama |
| | LVQAGGSLRLS | | PGKQRELV | | KNTVYLQLSSLKPEDTAV | | TQVTVSS | |
| | CAASG | | AVIS | | YYCNA | | | |
| 6fyu_I | EVQLVESGGGL | RTYA | MGWFRQAP | ALGTR | TYYSDSVKGRFTISRDNA | QGQWRAAPVAVA | EFWGQGT | Lama |
| | VQAGGSLKLSC | | GKEREFVA | | KNTEYLEMNNLKPEDTAV | AEY | QVTVS | |
| | AASG | | HIN | | YYCTA | | | |
| 6gci_B | QVQLVESGGG | ITFSSYT | MGWFRQAP | GSGSN | TYYADSVKGRYTISRDNA | RDDGRMYQGFYD | EYWGQGT | Lama |
| | LVQAGDSLRLA | | GKEREYIAR | | KNTAYLQMNSLKPEDTA | F | QVTVSS | |
| | CAASG | | IT | | NYYCAA | | | |
| 6gs7_H | QVQLQESGGG | RTFSSYN | MGWFRQAP | WTGRS | ADYPDSVKGRFTISRDNA | KQYGSRADYPWD | DYWGQG | Lama |
| | LVQAGGSLRLS | | GKEREFVG | | KNAVYLQMNSLKPEDTA | DY | TQVTVSS | |
| | CAGSG | | GIS | | VYYCAA | | | |
| 5lmw_A | QVQLVESGGG | LTFNRYN | MGWFRQAP | WSGDR | TYGTDSVKGRFAISRDNA | DRFLTRSVKY | DYWGQG | Lama |
| | LVQAGDSLRLS | | GREREFVA | | KNTMYLQMNSLKPEDTA | | TQVTVSS | |
| | CAASG | | SIS | | VYYCAA | | | |
| 6h15_D | VQLQESGGCL | STFSTYT | IGWFRQAP | WDGG | TYYADSVKGRFTISRDNA | RGRRYFDFTYSD | DYWGQG | Lama |
| | VQAGGSLRLSC | | GKEREFVA | Q | KNTVYLQMNSLKPEDTAV | VY | TQVTVS | |
| | AASG | | AIH | | YYCAA | | | |
| 6h16_B | EVQLQESGGG | RTFSIYT | IGWFRQAP | WSGGS | TYYADSVKGRFTISRDNA | ITYTRGIY | KYWGQGT | Lama |
| | LVQAGGSLRLS | | GKEREFVA | | KNTVYLQMNSLKPEDTAV | | QVTVSS | |
| | CAASG | | EIT | | YYCAA | | | |
| 4s11_A | QVQLQESGGG | RTFSSFV | MGWFRQAP | RSGSV | TRYADSAKGRFTISKDNA | DLHRPYGPGSQR | DTWGQGT | Lama |
| | LVQAGGSLRLS | | GKEREFVA | | KNTVSLQMDNLNPDDTA | TDDY | QVTVSS | |
| | CAASG | | SIS | | VYYCAA | | | |
| 3p0g_B | QVQLQESGGG | SIFSINT | MGWYRQA | SGGS | TNYANSVKGRFTISRDNA | KDYGAVLYEY | DYWGQG | Lama |
| | LVQAGGSLRLS | | PGKQRELV | | ANTVYLQMNSLKPEDTAV | | TQVTVSS | |
| | CAASG | | AAIH | | YYCNV | | | |
| 4qkx_B | QVQLQESGGG | SIFALNI | MGWYRQA | SGGT | TNYANSVKGRFTISRDNA | KDFGAIIYDY | DYWGQG | Lama |
| | LVQAGGSLRLS | | PGKQRELV | | ANTVYLQMNSLKPEDTAV | | TQVTVSS | |
| | CAASG | | AAIH | | YYCNV | | | |
| 6n51_D | QVQLVESGGG | RTFTSYA | MGWFRQAP | SSGGS | THYADSVKGRFTISRDNS | AMYGSRWPDWE | DYWGQG | Lama |
| | LVQAGGSLRLS | | GKERESVA | | KNTVYLQMNSLKPEDTAV | Y | TQVTVSS | |
| | CAASG | | AIS | | YYCAA | | | |
| 6nfj_E | QVQLVESGGG | RAIRSYF | MAWFRQAP | YIFN | TYYADSVKGRFTISRDNA | GVGASVSVSESW | NYWGQG | Lama |
| | LVQAGGSLRLS | | GKEREFVA | | KNTVFLQMNSLKPEDTAV | Y | TQVTVSS | |
| | CAASG | | AVE | | YYCAA | | | |
| 6r7t_A | QVQLQESGGG | FTFSTYA | MSWFRQAP | SSGGG | QSYADSVKGRFTISRDNA | AAWRVGTY | DYRGQGT | Lama |
| | LVQPGGSLRLS | | GKGLEWVA | - | KNTLYLQMNSLEPEDTAV | | QVTVSS | |
| | CAASG | | SIN | | YYCAA | | | |
| AAL31962.1 | QVQLQESGGG | APFREST | MAWYRQTP | SGGS | KTYGVSVQGRFTISRDSD | ALS | NTWGQGI | Lama |
| | LVQPGGSLRLS | | GKERETVA | | RRTVLLQMNNLQPEDTA | | QVTVSS | |
| | CAVSG | | FIT | | VYYCHR | | | |
| AAL31963.1 | QVQLQESGGG | EGFSNYP | MGWYRQA | EGGDR | TNYADAVKGRFTISRDNA | ARWDLGPAPF | GSWGQG | Lama |
| | LVQPGGSLRLS | | PGKQRELV | | KKTVYLQMSSLKPEDTAV | | TQVTVSS | |
| | CVVSG | | AAMS | | YYCNA | | | |
| AAL31964.1 | QVQLQESGGG | RTISTYS | MGWFRQAP | RTGSN | TLYADSVKGRFIISRDNAK | GLATWLATQTDN | DYWGQG | Lama |
| | TVQAGDSLRLS | | GKEREFVA | | NTMYLQMNSLKPADAAV | Y | TQVTVSS | |
| | CAASA | | HIS | | YYCAA | | | |
| 2wzp_L | QVQLQESGGG | RTGSNWC | MGWFRQLA | FDYDM | TYYADSVKGRFTVSRDS | RSGGFSSNRELY | DGWGQG | Lama |
| | LVQAGGSLRLS | | GKEPELVVA | | GKNTVYLQMNSLKPEDT | | TQVTVSS | |
| | CTASR | | LN | | AIYYCAA | | | |
| AAL31966.1 | QVQLQESGGG | RTGSNWS | MGWFRQLA | LDYDI | PYYADSVKGRFTVSTDS | RSGGFSSNRTYY | DYWGQG | Lama |
| | LVQAGGSLRLS | | GKEREFVV | | GKNTVYLQMNSLKPEDT | | TQVTVSS | |
| | CTASR | | ALN | | AIYFCAA | | | |
| AAL35838.1 | DVQLQASGGG | RTFSNYH | MGWFRQAP | WSGGN | TYYADSVKGRFTISRDNA | GSKYGGSWSRS | NYWGQG | Lama |
| | LVQAGGSLRLS | | GTEREFVS | | KNAVYLQMNSLKPEDTA | QDAY | TQVTVSS | |
| | CTASG | | SIK | | VYYCAT | | | |
| AAL35839.1 | EVQLQASGGG | RIFSNAA | MGWFRQAP | WSDGN | TYYADSVKGRFATSRDN | GIGTFGSSWTRA | RYWGQG | Lama |
| | LVQAGGSLRVS | | GKEREFVA | | AKNTVYLQMNSLKPEDTA | DRY | TQVTVSS | |
| | CAASG | | AIR | | VYYCAA | | | |
| AAL35840.1 | EVQLQASGGG | SIFSINT | LGWYRQAP | SGGA | TYYADSMKGRFTISRDNA | RVPL | DYWGQG | Lama |
| | LVQPGGSLRLS | | GKQRELVA | | KNTVYLQMNSLKPEDTAV | | TQVTVSS | |
| | CVASR | | WIT | | YYCNK | | | |
| AAL35841.1 | DVQLQASGGG | RSFSTYR | VGWFRQTP | WNGVK | TRYSDSMNDRFTVSRDN | DQRFDGDDWSPS | TRWGRGT | Lama |
| | LVQAEDSLRLS | | GKEREFVA | | TKNTVFLDLKSLKDEDTA | AF | QVTVSS | |
| | CALSG | | GIN | | VYYCAT | | | |
| AAL35842.1 | EVQLQASGGG | NTISGYA | TGWFRQAP | WSGYS | VYYAKSPKGRFTVSRDN | AVFVRTAGVPTLP | DYWGQG | Lama |
| | LVQAGGSLRLS | | GKEREFVA | | AKKNTVYLQMNSLKHED | GEY | TQVTVSS | |
| | CAASG | | AVT | | TAVYYCA | | | |
| AAL35843.1 | DVQLQASGGG | GSFSNYN | MGWFRQAP | WSGGR | IIVADSVKGRFTIFRDNAK | TKQFFPLSNESV | DYWGRGT | Lama |
| | LVQAGGSLRVS | | GKGREFVV | | STVYLQMNSLTPEDTAIY | WY | QVTVSS | |
| | CAASG | | GIG | | YCAA | | | |
| AAL35844.1 | DVQLQASGGG | RIPRNYP | IGWWRQTP | WTSGT | TYFADSVKGRFAISRDNA | SERDFYTRNYYFT | DYWGQG | Lama |
| | LAQAGRSLTLS | | GKKREFVA | | KKTATLQMNNLQLEDTAV | ESLY | TQVTVSS | |
| | CDVSG | | GIS | | YYCAT | | | |
| AAL35845.1 | DVQLQASGGG | ESIASFN | LGWFRRAP | RTGET | TDYADAVKGRFTISRDNI | DYNLGTFVTRKD | DFWGQGT | Lama |
| | LVQSGGSLRLI | | GKAREFVG | | KNMGYLQMDNLKPGDTA | SMY | QVTVSS | |
| | CEASG | | AVS | | TYYCAA | | | |
| AAL35846.1 | DVQLQASGGG | RTFSSVS | MGWFRQAP | WRGVS | TYYADSVKGRFTISRDNA | RRNFFGNNSAGQ | AYWGQG | Lama |
| | LVQAGGSLRLS | | GKEREFVA | | KNTGYLQMNSLKPEDTA | Y | AQVTVSS | |
| | CAASG | | AIN | | VYYCAA | | | |
| AAL35848.1 | DVQLQASGGG | RTFSSVT | MGWFRQAP | RNSGS | TYYADSVKGRFTIPRDNA | KASMYGSTLYPP | NYWGQG | Lama |
| | LVQAGGSLRLS | | GKEREFIAA | | KNTGYLQMNSLKPEDTA | TGY | TQVTVSS | |
| | CAASG | | MT | | VYYCAA | | | |
| AAL35857.1 | EVQLQASGGG | RTFSRFA | MGWFRRAP | WSGGT | AYGADSAKGRFTISREYA | GRAVSDY | DYWGQG | Lama |
| | LVQAGGSLRLS | | GKEREFVA | | KNIMYLQMNSLKPEDTAV | | TQVTVSS | |
| | CAASG | | AIS | | YYCAA | | | |
| AAL35850.1 | EVQLQASGGG | SIFSESA | MGWYRQA | LDGR | TNYAYYAEGRFTISRDNA | LRSRAVMDTIP | NYWGPGT | Lama |
| | LVQPGGSLRLS | | PGNERELV | | KNTVYLQMNNLEPGDTA | | QVTVSS | |
| | CATSG | | AAIT | | VYYCNA | | | |
| AAL35851.1 | DVQLQASGGG | RTFSSDA | MGWFRQAP | WSGGS | TYYADSVKGRFTISRDNA | DRRRYYSGSYPP | DYWGQG | Lama |
| | LVQAGGSLRLS | | GKEREFVA | | KNTVYLQMNSLKPEDTAV | SEY | TQVTVSS | |
| | CAASG | | AIS | | YYCAH | | | |
| AAL35853.1 | EVQLQASGGG | LPFSTYS | MGWYRQA | GGGN | TYHADSLKDRFTISRDND | DRDFTIVAGFIRS | EYWGEGT | Lama |
| | SVQTGASLRIS | | PGKEREFV | | KNTVYLQMNSLKPEDTAV | QYSPRAV | QVTVSS | |
| | CLASG | | AVIG | | YYCAA | | | |
| AAL35854.1 | EVQLQASGGG | RTFSTYT | MGWFRQAP | RNSVG | TYYRDSVKGRFTISRDNA | DPMYGRSVMSTR | NYWGQG | Lama |
| | LVQAGGSLRLS | | GKEREFVA | | KNTVYLQMNSLEPEDTAV | Y | TQVTVSS | |
| | CVASG | | AIS | | YYCAA | | | |
| AAL35855.1 | EVQLQASGGG | YTFSSHA | MGWFRQTP | ASGGN | QYYKYFAKGRFTISRDNA | ATKQFSNAYSDY | DYWGQG | Lama |
| | LVQAGGSLRLS | | GKDREFVS | | KNTVDLQMNSLRPEDAA | VHDY | TQVTVSS | |
| | CAVSG | | AIS | | VYYCAR | | | |
| AAL35859.1 | EVQLQASGGG | GTFTDYA | MGWFRQAP | WGGYS | TYYSDAVKGRFTISRDND | DPQLITTPEY | NYWGQG | Lama |
| | LAQAGGSLRLS | | GKEREFVA | | KNMSYLQMNNLKPEDTA | | TQVTVSS | |
| | CAASG | | AIN | | VYFCAA | | | |
| AAL35860.1 | DVQLQASGGG | NTISDYA | TGWFRQAP | RRT | GWQVYSDSVKNRFTVSR | SQDSGFDTPVTE | GYWGRG | Lama |
| | LVQAGGSLRLS | | GKEREFVG | | DSAKMYLQMNSLSPDDT | SHLY | TQVTVSS | |
| | CAASG | | SIG | | AVYYCAA | | | |
| AAL35872.1 | DVQLQASGGG | FTFDEHA | IGWFRQAP | TGGS | TWYAASVKGRFTVSRDD | LSQGAM | DYWGKGT | Lama |
| | SVQAGGSLRLS | | GKGLEYVS | | AKNTLYLQMNSLKPEDTG | | QVTVSS | |
| | CAASG | | HID | | LYYCAR | | | |
| AAL35873.1 | DVQLQASGGG | FTLDEHA | IGWFRQAP | IGGGR | TYYADSVKGRFTISGDNA | AHGGYGAF | GSWGQG | Lama |
| | SVQDGGSLRLS | | GKGFESVS | | KNTLYLQMNSLKPEDTAF | | TQVTVSS | |
| | CAASG | | TID | | YYPAR | | | |
| AAL40814.1 | EVQLQASGGG | RTFSSYG | MGWFRQAP | ESGAD | THYADFVRGRFTISGDNA | LDITTAA | SYWGQGT | Lama |
| | LVQTGGSLRLS | | GKEREFVA | | KNTVYLQMNRLTPEDTAV | | QVTVSS | |
| | CAVSG | | AMR | | YYCKA | | | |
| AAL40817.1 | EVQLQASGGG | RTFSSYS | MAWFRQAP | WRSSV | TAYADSVKGRFTISRDNA | EALPGTYGL | DYWGKGT | Lama |
| | LVQPGGSLRLS | | GKEREFVA | | KNTVYLQMNGLKPEDTA | | QVTVSS | |
| | CEASG | | AIN | | VYYCAR | | | |
| AAL40818.1 | DVQLQASGGG | STFSIGA | IGWYRQAP | GGGS | TYYTDSVKGRFTISRDNA | ILAGGLL | AFWGQGT | Lama |
| | LVQAGGSLRLS | | RKQRELVA | | RNTVYLQMNNLKPEDTA | | QVTVSS | |
| | CAASV | | GIS | | VYYCNA | | | |
| AAL40819.1 | EVQLQASGGG | STFSGND | IGWYRQAP | DGGY | TSYATSVKGRFTISRDNR | GGSSG | TFWGQGT | Lama |
| | LVQAGGSLKLS | | GKQRELVA | | KNTAYLQMNSLKPEDTAV | | QVTVSS | |
| | CTASG | | VIS | | YYCFA | | | |
| AAL40823.1 | EVQLQASGGG | PTSITYG | MAWFRQAP | PSGGA | AAYADSVKGRFTISRGVG | GTNWPPK | RYWGLGT | Lama |
| | LVQVGDSLKLS | | GKEREFVS | | KNTVYLQMDSLKVEDTAV | | QVTVSS | |
| | CAASG | | AVT | | YYCAA | | | |
| AAL40825.1 | EVQLQASGGG | RTFGSYT | MGWFRQAP | SAGR | TMYADSVKGRFTISRDNA | GTVLSVATGPY | GYWGQG | Lama |
| | LVQAGGSLRLS | | GKQRELIAR | | KSTVYLQMNSLKPEDTAV | | TQVTVSS | |
| | CVASG | | IN | | YYCAA | | | |
| AAL40826.1 | DVQLQASGGG | RTFSSYG | MGWFRQAP | WRGSS | TYYADSVKGRFTISRDNT | WGAGEDE | DYWGQG | Lama |
| | LVQAGGSLRLS | | GKEREFVA | | VNTVSLQMNNLKPADTG | | TQVTVSS | |
| | CVASG | | SIN | | VYYCAA | | | |
| AAL40830.1 | EVQLQASGGG | ITFSEKH | MAWFRQAP | RGGT | TNYGDSVKGRFTISRQKL | DFYGLGF | DYWGQG | Lama |
| | LVQAGGSLRLS | | GKQRELVA | | NNTIILQMNNLKPEDTAVY | | TQVTVSS | |
| | CVASG | | VIT | | YCAA | | | |
| AAL40831.1 | EVQLQASGGG | RTFSSYH | MGWFRQAP | WSGDT | TYYEASVKGRFTISRDNA | QTRPRPYGTSRA | GYWGQG | Lama |
| | LVQAGDSLRLS | | GKEREFVA | | KNTMYLQMNSLKPEDTA | EGDY | TQVTVSS | |
| | CAASG | | AIN | | VYRCAA | | | |
| AAL58846.1 | EVQLQASGGG | FKITHYT | MGWFRQAP | WGGD | TFYSNSVKGRFTISRDNA | GSTSTATPLRV | DYWGKGT | Lama |
| | LVQAGGSLRLS | | GKEREFVS | N | KNTVYLQMNSLKPEDTA | | QVTVSS | |
| | CAASG | | RIT | | DYYCAA | | | |
| AAL58847.1 | EVQLQASGGG | RTFSIYA | MGWFRQAP | RSGDV | TKYADFVKGRFSISRDNA | TWAYDTVGALTS | NFWGQGT | Lama |
| | LVQAGGSLRLS | | GKEREFVA | | KNMVYLQMNSLKPEDTA | GY | QVTVSS | |
| | CSASV | | GIN | | LYYCAA | | | |
| CAA10533.1 | LQASGGGLVQ | GLIFSSYA | MGWFRQAP | RGGKI | RHYADSVKGRFTISRDDA | DDIGGGWMYERS | NYWGQG | Lama |
| | AGGSLRLSCAE | | GKEREFVA | | KNTLYLQMNSLQPEDTAV | WAQSRRNY | TQVT | |
| | AS | | AIN | | YFCAA | | | |
| CAA15407.1 | QVQLQESGGG | PMSSSAV | MSWFRQAP | WSGGT | SYYANSVEGRFTISRDNA | TQSGSFYQV | DYWGQG | Lama |
| | LVQAGGSLTLS | | GKEQEFVA | | KNTMYLQMNKLKPEDTA | | TQVTVSS | |
| | CAASG | | RIR | | VYTCAL | | | |
| CAA15408.1 | QVQLQESGGR | IDVNRNA | MGWFRQAP | WSDAY | TDYADSVKGRFTISRDNN | GLLDVQYVRQAA | SYWGQGT | Lama |
| | SVQSGGSLRLS | | GTEREFVA | | KNTVYLQMGSLEAGDTA | GY | QVTVSS | |
| | CAASG | | GVR | | LYYCAA | | | |
| CAC07784.1 | QVQLQESGGG | RTSHGYG | MGWFRQIP | WSGRN | TYYADSVKGRFTISRDNV | RTVRVVDISSPVG | AYWGQGT | Lama |
| | LVQAGDSLRLS | GYG | GKERELVA | | KDMLYLQMNSLKPEDTA | F | QVTVSS | |
| | CAASG | | AIR | | VYTCAV | | | |
| CAA15412.1 | QVQLQESGGG | RATSGHG | MGWFRQVP | WSGKE | TWYKDSVKGRFTISRDNA | RPVRVDDISLPVG | DYWGQG | Lama |
| | LVQAGGSLRLS | HYG | GKEREFVA | | KTTVYLQMNSLKPEDTAV | F | TQVTVSS | |
| | CAASG | | AIR | | YYCAA | | | |
| CAA15414.1 | QVQLQESGGG | NIDRLYA | MAWYRQAA | LSGL | LTYADSVKGRFTVSRDNA | AYGNP | TYWGKGT | Lama |
| | LVQPGGSLRLS | | GKQRELVAI | | KNTVYLQMHSLKPEDTAV | | LVTVSS | |
| | CAASG | | AH | | YYCNR | | | |
| CAA15418.1 | QVQLQQSGGG | RTFSGHG | MGWFRQVP | WSGVT | TYYVDSVKGRFTISRDNA | RPVRVDDISTPDG | VYWGQGT | Lama |
| | LVQAGGSLRLS | GYG | GKEREFVA | | KSTVYLQMNSLKPEDTG | F | QVTVSS | |
| | CVASG | | AIR | | VYYCAV | | | |
| 1sjv_A | QVQLQESGGG | NTFSGGF | MGWYRQA | SRGI | TNYADFVKGRFTISRDNA | HYFR | SYWGQGT | Lama |
| | LVQAGESLKLS | | PGKQRELV | | KKTVYLEMNSLEPEDTAV | | QVTVSS | |
| | CAASG | | ATIN | | YYCYT | | | |
| CAA15420.1 | QVQLQESGGG | RASSGHG | MGWFRQVP | WSGKE | TWYVDSVKGRFTISRDNA | RPVRVDDISSPVG | DYWGQG | Lama |
| | LVQAGGSLRLS | GYG | GKEREFVA | | KSTVYLQMNSLKPEDTAV | F | TQVTVSS | |
| | CAASG | | AIR | | YYCAV | | | |
| CAB40421.1 | LQESGGGLVQ | SIFSIDV | MGWYRQA | RGGS | TNYAAFVKGRFTISRDNS | EGIPETLG | SRWGQG | Lama |
| | AGGSLRLSCAA | | PGKQRELV | | KKTLYLQMNSLKPEDTAV | | TQVT | |
| | SA | | AQIT | | YYCRA | | | |
| CAB40422.1 | LQESGGGLVQ | SIVRGNT | MAWYRQAP | SGGS | IDYSDSVKGRFTISRDSLK | DVWYGSTWR | NYWGQG | Lama |
| | PGGSLKLSCAV | | GKERELVA | | NTVCLQMNSLKPEDTAV | | TQVT | |
| | FG | | HYT | | YYCNA | | | |
| CAB40423.1 | LQESGGGLVQ | SFSNMNV | MGWYRQA | SNGY | INYGDSVEGRFTISGSVA | KWIGIVQR | DYWGQG | Lama |
| | PGGSLRLSCAA | | PGKQRELV | | ENTVYLQMNSLRPEDTA | | TQVT | |
| | SG | | AGVN | | VYYCNA | | | |
| CAB40424.1 | LQESGGGLVQ | SIGSINA | MGWYRQA | PGGS | TEYADSVKGRFAISRDNA | INWKS | DYWGQG | Lama |
| | PGGSLRLSCAA | | PGKQRELV | | KTTVYLQMNSLKPEDTAV | | TQVT | |
| | SG | | AQFT | | YYCYC | | | |
| CAB40425.1 | LQESGGGWVQ | MIFDFND | MGWYRQA | RFGR | TNYIDSVKGRFSISNDPA | DPPL | FAWGQGT | Lama |
| | PGGSLRLSCAA | | PGKERELV | | KWTVYLQMNSLKPEDTA | | QVT | |
| | SE | | ASIS | | VYSCNT | | | |
| CAB40426.1 | LQESGGGLVQ | SIYSLDA | MGWYRQA | SRGI | TTYADSVKGRFTISRDNA | VVYYGDWEGSEP | DYWGQG | Lama |
| | PGGSLRLSCAA | | PGEQRELV | | KNTVTLQMNILKPEDTAV | VQHEY | TQVT | |
| | TG | | ATWT | | YYCHA | | | |
| CAB40427.1 | LQESGGGSVQ | TIHSIVD | MAWYRQFP | SGGN | TNYVDSVKGRFTISRDDA | RIRRHESGYAY | DSWGQG | Lama |
| | AGGSLRLSCVA | | GKQRELVA | | KNTIYLQMNSLKPEDTAV | | TQVT | |
| | SG | | ARN | | YYCNA | | | |
| CAB40428.1 | LQESGGGLVQ | SIFSVNA | MGWYRAAP | SGGS | TNYADSVKGRFTISRDNA | CRVGNWRLE | GSWGQG | Lama |
| | AGGSLRLSCAA | | GRQRELVA | | KNTMYLQMNDLKPEDTA | | TQVT | |
| | SG | | SIT | | VYYCTN | | | |
| CAB40429.1 | LQESGGGLVQ | NVFMIKD | IGWYRQAP | DGGT | TNYADSVKGRFTISRDNA | DSWRIGAK | DYWGQG | Lama |
| | PGGSLRLSCAA | | GKQRELIAA | | KNTVFLQLNSLKPEDTAV | | TQVT | |
| | SG | | IT | | YYCHA | | | |
| CAB40430.1 | LQESGGGLVQ | GIFSIND | MVWYRQAP | RIFS | EIYADSVKGRFTISRDNA | PTG | NYWGQG | Lama |
| | TGGSLTLSCTA | | GKQREWVA | | KNIMYLQMNSLRPEDTAV | | TQVT | |
| | SG | | TIT | | YYCSA | | | |
| CAB40431.1 | LQESGGGLVQ | GTNT | MGWFRQAP | WAGDI | KSYGDSVKGRFTISRDNA | SPRLRVEIKAGGY | DFWGQGT | Lama |
| | PGGSLRLSCAA | | GKGREFVA | | KNTVYLQMNSLIPEDTAV | | QVT | |
| | SE | | ALS | | YYCAA | | | |
| CAB40432.1 | LQESGGGLVQ | ITFSNDA | SGWSRQAP | SGGT | TVIADSVKGRFTISRDDAK | DSNGR | TYWGQGT | Lama |
| | AGGSLRLSCAA | | GKQLDFIAR | | NTVYLHMNSLKPEDTAVY | | QVT | |
| | SG | | IR | | FCNA | | | |
| CAB40433.1 | LQESGGGLVQ | STFSIST | MGWHRQA | SLGT | TDYADSVKGRFTISRDNA | DGLYGFTGK | NYWGQG | Lama |
| | AGGSLRLSCGA | | PGKEREYV | | KNTVYLQMNSLKPEDTAV | | TQVT | |
| | SG | | ANIG | | YYCNQ | | | |
| CAB40434.1 | LQESGGGLVQ | RTWNDLD | MGWFRQAP | S | GGTPHYADSRFTLSRDH | GAYSTRPEAY | RYWGQG | Lama |
| | AGGSLRLSCVA | | GNQREFLA | | AKNLVYLQMNSLKPEDTA | | TQVT | |
| | QG | | AIT | | RYYCAA | | | |
| CAB40435.1 | LQQSGGGFVQ | RTSTVNG | MGWYRQA | PRGT | TQYHDNVKGRFTISRDNT | DVGPT | VKVGQGT | Lama |
| | PGGSLRLSCTA | | PGKLRERV | | RTTAYLQMNDLQPDDTA | | QVT | |
| | SG | | AVVM | | NYYCFA | | | |
| CAB40437.1 | LQESGGGLVR | SDINFNV | MGWYRQA | SGGS | TDYADSVKGRFTISRHNA | DIFFV | NYWGKGT | Lama |
| | PGESLRLSCVV | | PGQQRELV | | NNMVYLQMNSLKPEDTA | | LVT | |
| | SG | | ATIT | | VYYCNA | | | |
| CAB40438.1 | LQESGGGLVQ | RTLENYR | VAWFRQFP | WSGDR | RYYTEAVQGRFNISRDNA | AEMYGSLARRDY | RNWGQG | Lama |
| | AGGSLRLSCVA | | GKERELIAV | | ENTVYLLMNSLKPEDTAV | | TQVT | |
| | SG | | IN | | YYCAA | | | |
| CAB40440.1 | LQESGGGLVQ | LAFENYF | VSWFRQPP | YSSSS | TNYADSVKGRFTISRDNA | CSDYYCSGVGA | VYWGQGT | Lama |
| | AGGSLRLSCVA | | GDEREFVA | | VNTAYLQMDNLAPGDTA | | QVT | |
| | SG | | AIT | | VYFCAA | | | |
| CAB40441.1 | LQESGGGLVQ | FTLDLYT | IGWFRQAP | SSDGS | TYYADSVKGRFTISRDNA | DSNYCSGYGPS | DYWGQG | Lama |
| | PGGSLRLSCAA | | GKEREVVS | | KNTVYLQMNSLKPEDTAV | | TQVT | |
| | SG | | CIS | | YYCAT | | | |
| CAB40442.1 | LQESGGGSVQ | FILDYYA | IIWFRQAPG | SSDGT | TYYADSVKGRFTLSRDNA | PEGYYCPTRPLG | EYRGKGT | Lama |
| | PGGSLRLSCST | | KEREAVSCI | | KNTVFLQMDSLKQEDTAL | L | LVT | |
| | SG | | S | | YWCAR | | | |
| CAB40443.1 | LQQSGGRSVQ | RALNTLH | MGWFREAP | RSGET | TWYADFVEGRFTVFRDN | SMSLRPLDPASY | DYWGQG | Lama |
| | AGGSLRLACTA | | GKGREFVG | | AKNAVYLEMTSLKPEDTA | SPDIQPY | TVVT | |
| | SG | | SIS | | SYYCAA | | | |
| CAB40446.1 | LQESGGGLVQ | PTLGSYV | MGWFRQAP | WRGG | TYYAQFAKGRFAISRVTG | KASPAVVTTKQY | DYWGQG | Lama |
| | AGGSLRLSCAA | | GKEREYVG | N | EKMVYLQMNDLNPEDTA | | TQVT | |
| | SG | | SIT | | VYYCAA | | | |
| CAB40448.1 | LQESGGGLVQ | GTFSEHY | MAWFRQAP | WSRGS | TFYADSAKGRFTISRDKG | GSNAGLLTYSDY | EFWGQGT | Lama |
| | PGGSLRLSCAA | | GNQRVFVA | | EKTMYLEMNALKPDDTA | DYLY | QVT | |
| | TG | | VIN | | VYYCAA | | | |
| CAB40449.1 | LQESGGGLVQ | RTSDIYG | MEWHRQVP | WSDGA | TNYADSVKGRFTVSRDN | QPFGASSY | RYWGVGT | Lama |
| | AGNSLRLSCAA | | GKEREFVVI | | AKNTMYLQMNSLKPEDT | | QVT | |
| | SG | | LS | | AVYYCAL | | | |
| CAB40450.1 | LQESGGGLVQ | RIGSISG | MSWYRQVP | EGGA | RNYADFVEGRFTISKDNA | RRRFGGQ | ELWGQGT | Lama |
| | AGGSLKLSCAA | | GAQREFVA | | KSTLYLQMNNLKSDDTG | | QVT | |
| | PE | | TIT | | VYYCKL | | | |
| CAB40451.1 | LQESGGGLVQ | FTFDDYA | ICWFRQAP | KEYAK | TYYTDAVEGRFTISSDDA | KRGWYFPLDLSE | DLWGQGT | Lama |
| | TGGSLRLSCTA | | GKGREGVS | | KNTVYLQMNNLIPEDTAF | Y | PVT | |
| | SG | | CIS | | YYCAR | | | |
| CAB40452.1 | LQQSGGGLVQ | SITSIRS | MGWYRQA | TGGA | TNYADSVKDRFTISRDNA | LLYYSDYENE | NYWGRGT | Lama |
| | PGGSLRLSCAA | | PGKQREFV | | EDTVYLQMNSLKPEDTAI | | QVT | |
| | SG | | AHIS | | YYCNA | | | |
| CAB40454.1 | LQESGGGLVQ | NMFSINA | MGWYRQA | YLGR | TNYTDSVKGRFTISRENA | EATRGWGELRGR | DYWGQG | Lama |
| | PGGSLRLSCAA | | PGKQRELV | | KNTVYLQMSSLKPEDTAI | RH | TQVT | |
| | SG | | ASIT | | YYCNA | | | |
| CAB40455.1 | LQESGGGLVQ | RTFSNYA | MGWFRQAP | RSGTM | TYYADFVKGRFSISRENV | DSTKGWDLRDH | DFWGRGT | Lama |
| | AGGSLRLSCAA | | GKEREFVA | | KNTVYLQMNSLKPEDTAV | | QVT | |
| | SG | | GIS | | YFCNA | | | |
| CAB40456.1 | LQQSGGGLVQ | IIFSVRA | MGWYRQA | RGGS | TNYADSVKGRFTISRDRA | NVLTRALVSG | DYWGQG | Lama |
| | AGGSLRLSCAA | | PGKQRELV | | KNTAYVQMNSLKPDDTA | | TQVT | |
| | SG | | AVIL | | VYYCNA | | | |
| CAB40457.1 | LQESGGGLVQ | SIFSINT | MRWYRQAP | SGGS | TNYADSVKGRFTISRDNA | DGIHYYSDYDAPP | DYWGQG | Lama |
| | AGGSLRLSCAA | | GKHRELVA | | KNTVYLQMNSLKPEDTAV | IREY | TQVT | |
| | SG | | TIT | | YYCNA | | | |
| CAB40458.1 | LQESGGGLVQ | SIFSRNA | MAWYRQAP | VGGG | TTYSGSVKGRFTISRDNA | DAPAGYYPK | AYWGQGT | Lama |
| | PGGSLRLSCAA | | GKQRELVA | | KNTVYLQMNSLKPEDTAV | | QVT | |
| | SG | | AIA | | YYCNA | | | |
| CAB40459.1 | LQESGGGLVQ | RTLSRYT | MAWIRQAP | WTDGS | TYYEDTMKGRVAISRDNV | GSNIGGSRWRY | DYWGQG | Lama |
| | TGASLRLSCVA | | GKGREFIGR | | KNTIYLQMDNLKPEDTAV | | TQVT | |
| | SG | | IT | | YYCAA | | | |
| CAB40461.1 | LQESGGGLVPT | NIFSLNT | MRWYVQTP | SRKI | PKYADSVEGRFTISRDNA | DVHDGMQLR | NYWGQG | Lama |
| | GGSLTLSCEVS | | GNEREMVA | | LNTIDLQMNSLKPEDTGV | | TQVT | |
| | G | | SIT | | YYCNG | | | |
| CAB40462.1 | LQESGGGVVQ | TFQRINH | MAWYRQIP | NDGR | LNYAASVKGRFTISRDSA | DLDVGFITE | NYWGPGT | Lama |
| | PGESLTLSCVA | | GKEREQVAI | | ENTAHLQMKSLRPEDTA | | QVT | |
| | SG | | IH | | VYYCNV | | | |
| CAB40463.1 | LQESGGNFMQ | RAFDAYH | MGWFRQTP | RSGTS | TYYADSVKGRFTISRDNA | ELHNSDPFNPSE | DYWGQG | Lama |
| | AGGSVRLSCAA | | EKQREFVA | | KDTVYLQMNNLKPEDTA | Y | TQVT | |
| | SG | | FIH | | MYYCAA | | | |
| CAB40464.1 | LQESGGGLVQ | FTLDNYA | IGWFRQAP | TDVDS | KYYASALKGRFTISSDSA | SSRHYGANCLY | AFWGHGT | Lama |
| | AGGSLKLSCET | | GKDREEVA | | KNTVYLQMNSLKPEDTAV | | QVT | |
| | SG | | CIS | | YYCGA | | | |
| CAB40466.1 | LQESGGGLVQ | IIFSLNH | MGWYRQA | TGGG | TYYADSVKGRFSISRDNA | RAVDELLT | MNWGQG | Lama |
| | AGGSLRLSCAA | | PGKQRELV | | KNTVYLQMNSLKPEDTAV | | TQVT | |
| | SG | | ALIH | | YYCNS | | | |
| CAB40467.1 | LQESGGGLVQ | SSLSNYT | MGWFRHFP | WSGGR | THYTESVKGRFTTGRDTA | ASRWSWNPTSDD | IYWGQGA | Lama |
| | AGGSLRLSCAA | | GKDREFVA | | KNTVYLQMNRLKPEDTAI | Y | QVT | |
| | SG | | VVK | | YYCAA | | | |
| CAB40469.1 | LQESGGGLVQ | STFTNYA | MGWYREAP | KVGD | TDYAGSVKGRFTISRDKS | FPAIVGPF | TSWGQGT | Lama |
| | PGGSLRLSCAA | | GKQRELVA | | GNTVFLQMNSLKPEDTA | | QVT | |
| | SG | | RIS | | VYYCNT | | | |
| CAB40471.1 | LQESGGGLVQ | RTFSSFV | MGWFRQAA | ERGSL | IHYADFVKGRFTISRDNA | VIGGRWPPSEY | AYWGQGT | Lama |
| | TGGSLRLSCAT | | GTEREFVS | | ENTVSLQMNSLKPEDTAV | | QVT | |
| | SG | | AIS | | YYCAA | | | |
| CAB40472.1 | LQESGGGTVE | NSFNNYV | LAWYRQAP | NNGG | TDYADSVKGRFAISRDNA | RRWIPPGP | INWGQGT | Lama |
| | AGGSLRLSCAA | | GKPRELVA | | ATTLYLQMDHLTPEDTAV | | QVT | |
| | SV | | GYT | | YYCTA | | | |
| CAB40474.1 | LQQSGGGLVQ | VGFSVTN | LGWFRQAP | WSGG | SDYVDSVKGRFTISRDNA | QAAGLSRDAHEY | KYWGQGT | Lama |
| | AGDSLRLSCTY | | GKEREFVA | | KNTATLQMNSLKPEDTAV | | QVT | |
| | SG | | SMM | | YFCAA | | | |
| CAB40480.1 | LQQSGGGLVQ | IIASDSA | MAWYRQAP | AGGL | SNYEDFVNGRFTISRDNA | GQSYIRSYYNPG | DYWGQG | Lama |
| | AGGSLRLSCVA | | GKGREYVA | | RNTMYLQMNSLKPEDTA | | TQVT | |
| | SG | | HIT | | VYYCNV | | | |
| CAB40481.1 | LQESGGGLVQ | SIFSMNT | VSWYRQAP | GDGR | TSYEDFVKGRFTISRDIAK | WISADASMMTT | NYWGQG | Lama |
| | PGGSLRLSCAA | | GNQRELVA | | NTVYLQMNNLKPEDTAV | | TQVT | |
| | SG | | SIR | | YYCNA | | | |
| CAB40482.1 | LQESGGGLVQ | SGISFNA | MGWYRQA | RTGS | TNYADSVKGRFTISRDNA | GLEGLGYRLH | DYWGQG | Lama |
| | PGGSLRLSCTE | | PGKRRELV | | KNTVYLQMNSLKPEDTAV | | TQVT | |
| | SG | | AAIT | | YYCNL | | | |
| CAB40483.1 | LQESGGGLVQ | STFSIDA | MGWYRQA | NDGS | TNYADSVKGRFTVSRDY | EWAAMK | DYWGQG | Lama |
| | AGGSLRLSCAA | | PGKQRELV | | GKNTVYLQMNSLKPEDT | | TQVT | |
| | SG | | ALMG | | AVYYCYA | | | |
| CAB40485.1 | LQESGGGLVQL | SIFSINR | VAWFRQAP | YSGS | TSYVDSVKGRFTISGDNA | ELPRD | VYWGQGT | Lama |
| | GGSLRLSCAAS | | GKQREFVA | | WNPVYLQMNSLKPEDTA | | RVT | |
| | G | | HIT | | VYYCYA | | | |
| CAB40487.1 | LQESGGGLVQ | NIFRINA | MGWYRQA | RGSS | TNYADSVKGRFTISRDNA | QGRYYGGTYDPT | DYWGQG | Lama |
| | AGGSLNLSCTA | | PGKQRELV | | KDTVTLQMNSLKPEDTAV | Y | TQVT | |
| | VG | | AQIS | | YYCNA | | | |
| CAB40488.1 | LQQSGGGLVQ | NASGNAFR | LGWYRQAP | SNGN | THYVDSVKGRFTISKDNA | DNNPIVYRPN | DYWGQG | Lama |
| | PGGSLRLSCAT | INA | GKQRELVA | | KNTVYLQMSSLKPEDTAV | | TQVT | |
| | SG | | LIA | | YTCAV | | | |
| CAB40489.1 | LQESGGGLVQ | SISTIYV | MGWYRQA | NGIY | TNYADSVKGRFTISRDDA | AIYTPGRGEF | DYWGQG | Lama |
| | AGGSLRLACAA | | PGKQRELV | | KNTLYLQMSSLKPEDTAV | | TQVT | |
| | SV | | VSVQ | | YYCNA | | | |
| CAB40491.1 | LQESGGGLVQ | STGSIVT | MGWWRQA | NRGT | TDYGDSLKGRFTISRDNA | HGIFGSSWTT | DYWGQG | Lama |
| | AGGSLTLSCEI | | VGKQREFV | | KNTLYLQMNSLQAGDTA | | TLVT | |
| | SG | | AWIN | | VYYCNA | | | |
| CAB40492.1 | LQESGGGWVQ | SGFRGNV | MAWYRQTP | SDDR | TNYADSVRGRFTISRDNA | DTGGPYGSRN | PYWGRGT | Lama |
| | PGGSLTLSCDA | | GKRRELVA | | KNMMHLQMNSLKPEDTA | | QVT | |
| | SG | | SIT | | VYYCHV | | | |
| CAB40493.1 | LQESGGGLVQ | STFSINP | MGWYRQA | TSTTTN | TNYGDSVKGRFTISRDNA | NARRLYSGSYL | DHWGQG | Lama |
| | AGGSLRLSCAA | | PGKQREFV | Y | RNTVYLHLQMNSLKPEDT | | TQVT | |
| | SG | | ADIY | | AVYYC | | | |
| CAB40494.1 | LQESGGGLVQ | RTFSLYT | MGWYRQA | SQ | GRTNYADSVKGRFTMSR | KLFVGA | DSWGKGT | Lama |
| | AGGSLRLSCVA | | PGKQRELV | | VKNILYLQMDSLKPEDAA | | LVT | |
| | SG | | ASIS | | VYYCHA | | | |
| CAB40495.1 | LQESGGGLVQ | FSLDDYD | IGWFRQAP | FLGHT | TNYADSVKGRFTISRDIAK | RREAPPY | AYWGQGT | Lama |
| | AGGSLRLSCAA | | GKQRELVA | | NSVYLQMNSLKPEDTAV | | QVT | |
| | SG | | SIS | | YYCNA | | | |
| CAB40496.1 | LQQSGGGLVQ | STFSINA | MGWYRQA | SSSS | TNYGDSVKGRFTISRDNA | RRPSAWGTD | NYWGQG | Lama |
| | AGGSLRLSCAA | | PGKQREFV | | KNTVYLQMNSLKPEDTAV | | TQVT | |
| | SG | | AVIS | | YYCNT | | | |
| CAB40497.1 | LQESGGDLVQ | SIDTINT | MAWYRQIP | NKGS | SNYADSVKGRFTISRDNA | EGIMMHNFNWYP | QSWGQG | Lama |
| | PGGSLRLSCVA | | GNQREFLA | | KNTVYLQMNSLKPEDTAV | | TQVT | |
| | PG | | AET | | YYCNA | | | |
| CAB40502.1 | LQESGGGLVQ | RSISDYT | MAWFRQVP | WNGN | LYYTESVKGRFTSSRDNV | RFRLNGYTPSYK | RYWGQG | Lama |
| | AGDSLKLSCVA | | GKEREFVA | | KNTVYLQMNSLKPEDTAV | EDDY | TQVT | |
| | SG | | VVT | | YYCAA | | | |
| CAB40503.1 | LQESGGGLVQ | PISGINR | MNWYRQAP | NDRR | INYAYSVRGRFTISRDNA | LISSVSRNGPFY | DYWGQG | Lama |
| | AGESLRLSCAA | | GKERELVA | | KNAVYLQMNSLKSEDTA | | TQVT | |
| | SG | | SIT | | RYYCYA | | | |
| CAB40504.1 | LQESGGGLVQ | RTISSYF | MAWFRQAP | RSGRG | TDYADSVKGRFTISRDNA | GDYFRSAAQFY | NYWGQG | Lama |
| | AGDSLRLACAA | | GKEREFVAL | | KNTVYLQMNSLKAEDTAV | | TQVT | |
| | SG | | IN | | YYCAA | | | |
| CAB40505.1 | LQESGGGLVQ | TTLDYYA | IGWYRQAP | SSGGA | TYYVDFVKGRFTISRDNA | LRDSYCGATLNY | DYWGKGT | Lama |
| | AGGSLRISCAA | | GKGREGVS | | KNTVYLQMNSLKPEDTAV | YYGI | RVT | |
| | SG | | CIS | | YYCVA | | | |
| CAB40508.1 | LQESGGGLVQ | STIENYA | MAWYRRVP | SGGS | TGYEASVEGRFTISRDNA | YPPL | IAGGQGT | Lama |
| | AGGSLRLSCVA | | GKDREMVA | | KQSVYLQMDSLKPEDTA | | QVT | |
| | SG | | RIT | | VYSCNT | | | |
| CAB40509.1 | LQESGGGLVQ | KFFEYYY | MDWFRQAP | SAGI | IDYADSVKGRFTISRDNA | DAPVDDGGSSR | DYWGQG | Lama |
| | AGGSLKLSCTA | | GKERERVA | | KNTVYLQMTDLKLEDTAV | | TQVT | |
| | SG | | AIS | | YYCHA | | | |
| CAB40510.1 | LQESGGGLAQ | VTLDLYA | IGWFRQAP | SSDGT | PYYADSVKGRFTISRDNA | STLRDYSDNYYFP | DYWGQG | Lama |
| | PGGSLRLSCVA | | GKKREWVS | | KDTVYLQMNSLKPEDTAV | HDYEY | TQVT | |
| | SG | | CIS | | YYCAA | | | |
| CAB40511.1 | LQESGGGLVQ | DTYKIYV | AGWVRQAP | WSGGF | GSYAGSVKGRFTISRNNA | HRARVTLTS | DYWGQG | Lama |
| | AGGSLTLSCAV | | GKERELVG | | KTMLYLQMSSLKPEDTAV | | TQVT | |
| | SG | | DIN | | YYCVA | | | |
| CAB40512.1 | LQESGGGLVQ | FTLENNM | IAWFRQAP | VDGG | IYYTESVKGRFTMSKDNA | YVGREFCSRSSV | DTWGQGT | Lama |
| | TGGSLRLSCAA | | GKGREWVS | | KNAVYLQMNSVNTEDTA | DF | QVT | |
| | PG | | CAA | | DYYCAA | | | |
| CAB40513.1 | LQESGGDLVQ | SFFKINA | MRWYRQAP | SGGA | TDYADSVRDRFTISMDNA | TTTRSYDDTYRNS | NYWGQG | Lama |
| | AGGSLRLSCAA | | GKQRELVA | | KNTVYLQMNSLKPEDTAA | WVY | TQVT | |
| | SG | | RIT | | YYCAA | | | |
| CAB40515.1 | LQQSGGGSVQ | FSFGDYA | VGWFRQAP | DNGGI | TSYVGSAKGRFTISRDNA | SDYRLGCDYVSD | GSWGQG | Lama |
| | PGGSLRLSCAA | | GKEREQVS | | KKMVYLQMNSLKTEDTA | | TQVT | |
| | SG | | CIN | | VYYCVA | | | |
| CAB40516.1 | LQESGGGSVQ | RTDRSNA | MGWFRQAP | WSGGT | TSYVDSVKGRFIISRDNV | ARTVDPLFRESEY | DYWGQG | Lama |
| | AGGSLRLSCTA | | GKEREFVA | | KKTLYLQMNSLKPEDTAV | | TQVT | |
| | SG | | TIS | | YYCAA | | | |
| CAB40517.1 | LQESGGGLVQ | SISSINV | MGWYRQA | HSGGH | TDYAESVKGRFTISGDAA | VSQWR | AYWGQGT | Lama |
| | AGGSLRLSCAA | | PGKQREWV | SGG | KNTVYLQMNSLKPEDTAV | | QVT | |
| | SE | | ATIV | | YYCNQ | | | |
| CAB40518.1 | LQESGGGSVQ | GAFSGLV | MGWFRQVP | RHGDT | PSYADAVKGRFSISRDNA | RRAFRLSSDY | EYWGQGT | Lama |
| | AGGSLRLSCAA | | GKEREFVA | | KNTVYLQMNSLKPDDTA | | QVT | |
| | SG | | QIN | | VYFCAS | | | |
| CAB40519.1 | LQESGGGLVQ | TIFAVHT | MGFYRQSP | TYRAN | TDYGEPVRGRFTLAKDN | ENYRRWPSTTPE | SYWGQGT | Lama |
| | PGGSLRLSCVA | | GKQRELVAI | | ARNTVTLQMDSLKPEDTA | | QVT | |
| | SG | | HN | | VYYCAL | | | |
| CAB40520.1 | LQESGGGLVQ | LTFGTHV | VGWFRQAP | YNVGS | LLYADSVSGRFTISRDNT | ERAERVADANFV | EYWGQGT | Lama |
| | AGGSLRLSCTA | | GNEREFVA | | KNTVYLQMVSLRPEDTAV | | QVT | |
| | SG | | AIS | | YYCGA | | | |
| CAB40521.1 | LQASGGGSVQ | STLSRYS | MGWFRQG | WSGGR | TLYGDAVKGRFTVSTENA | VRLGDSTVD | EYWGQGT | Lama |
| | AGGSLRLSCSV | | PGKEREFV | | KKMVYLEMTNLTPEDTAV | | QVT | |
| | SG | | AALL | | YYCNA | | | |
| CAB40522.1 | LQASGGGLVQ | FSFDDDYV | IGWFRQAP | WHDSH | INTEDSVKGRFTISGDNA | GSSGLKGPY | DYWGQG | Lama |
| | AGGSLRLSCAA | | GKGREGIA | | KNAVYLQMNSLNPEDTA | | TQVT | |
| | SG | | CIR | | VYFCAA | | | |
| CAB40524.1 | LQASGGGSVQ | SIFMINA | MGWYRQA | SGGI | TDYADSVKGRFTITRDNA | IQLTEWGLAVPEY | DYWGQG | Lama |
| | AGGSLTLSCAA | | PGKEREAL | | KNMVFLQMNSLKPEDTA | | TQVT | |
| | SE | | ASIT | | VYYCNA | | | |
| CAB40525.1 | LQASGGGLVQ | RTLDRYG | IGWLRQAP | ATAGR | TYYADFVKGRFTISRDKV | REFESCSEYAGG | DYWGQG | Lama |
| | PGGSLRLSCAA | | GKDREWVA | | KNTVDLQMNRMTPGDTA | Y | TQVT | |
| | SR | | CIT | | VYFCAS | | | |
| CAB40526.1 | LQASGGGLVQ | TLDTYA | IGWFRQAP | GEDDT | TYYVDSLKDRFTISRDNA | SRGNFGSGWYC | DYWGKGT | Lama |
| | PGGSLRLSCTS | | GKGREAVS | | KNTVSLQMNSLEPDDTA | GNDPQYYYGM | QVT | |
| | GV | | CIS | | VYFCAA | | | |
| CAB40527.1 | LQASGGGLVQ | PAFSAYA | MAWFRQAP | RDSRF | TTYLDSVRDRFAISRDNV | DFHTRGTLSRDE | TYWGQGT | Lama |
| | AGGSLRLSCAY | | GEEREFVA | | KNTVYLQMNSLKPDDTA | ADY | QVT | |
| | SG | | AIN | | VYSCAR | | | |
| CAB40528.1 | LQASGGGLVQ | FTLDNYA | IGWFRQAP | TSDGY | TLYSDSAKGRFTISYDND | EGAIHYCSGDFN | GLRGQGT | Lama |
| | AGGSLRLSCAA | | GKEREGVS | | KNTVYLQMNNLKPEDTA | | QVT | |
| | SG | | CIK | | VYYCAT | | | |
| CAB40529.1 | LQASGGGLVQ | RTFSSYA | IGWFRQDP | GTTRS | TVYGDSVKGRFTISRDNA | TAGSYLAPNSF | GYWGQG | Lama |
| | AGGSLTLSCAA | | GKEREFVA | | ENTVYLQMNSLKPEDTAV | | TQVT | |
| | ST | | AVS | | YYCAA | | | |
| CAB40530.1 | LQASGGGLVQ | GLTFSTYA | MGWFRQAP | RGGKI | SHYADSVKGRFTISRDDA | DDIGGGWLYERS | NYWGQG | Lama |
| | AGDSLRLSCTE | | GKEREFVA | | KNTLYLQMNSLQPEDTAV | WALSRRSY | TQVT | |
| | AS | | AIN | | YFCAA | | | |
| CAB40532.1 | LQASGGGLVQ | RTFSSYA | MGWFRQAP | WSGDS | THYADSVKGRFAISRDNA | DRDTGIGAY | DYWGQG | Lama |
| | AGGSLRLSCVA | | GKEREFVA | | KNTLYLQINSLKSEDTAVY | | TQVT | |
| | SG | | AIH | | YCAK | | | |
| CAB40533.1 | LQASGGGLVQ | RAFESYA | VGWFRQAS | WSGGS | TEYADSAKGRFTISRDNA | TARPFLGVPRITR | EYWGQGT | Lama |
| | AGGSLRLSCAA | | GKEREFVA | | KNTEYLQMNSLKPEDTAL | PNEY | QVT | |
| | SG | | AIN | | YYCAA | | | |
| CAB40535.1 | LQASGGGLVQI | RSVTSINL | ISWYRQAP | RSGL | ITFADSVKGRFSISRDSDK | EGTWYSGPRG | GYWGQG | Lama |
| | GGSLRLSCAAS | | GKQRELVA | | NTVYLQMNNMQPEDTAV | | TQVT | |
| | G | | KVP | | YYCNA | | | |
| CAB40536.1 | LQASGGGLVQ | FTFDDTG | IGWFRQAP | SRDGS | THYAHSVKGRFTITSDNA | EDHKSAASVCNS | DHWGQG | Lama |
| | AGGSLRLSCAA | | GKKREGVA | | KNTVYLQMNSLKPEDTAV | WLN | TQVT | |
| | SG | | CIS | | YICAA | | | |
| CAB40537.1 | LQASGGGLVQ | GTSNSYR | MGWFRQAP | RSGGS | TLYADAAKGRFTISRDNA | DLTDIWEGIREYD | AYWGQGT | Lama |
| | AGGSVRLTCAA | | GKEREFVA | | KNTAFLQMTSLKPEDTAV | EY | RVT | |
| | SG | | SIS | | YYCAA | | | |
| CAB40538.1 | LQASGGGLVQ | PTFSIYA | MGWFRQAP | WSGSS | TRYADSLKGRFTISRDNA | SDGALVVRSDMY | DYWGQG | Lama |
| | AGGSLRLSCAA | | GKEREFVA | | KNTVYLQMNSLKPEDTAV | | TQVT | |
| | ST | | AIH | | YYCAA | | | |
| CAB40716.1 | LQESGGGLVQ | RTFSRYN | MGWFRQAP | WNDG | GADYADSVKGRFTISRDA | DDIFATPGKY | DYWGQG | Lama |
| | AGGSLKLSCTA | | GKEREFVA | | DGTLYLQMNSLKPEDTAV | | TQVTVST | |
| | SR | | AIR | | YYCAL | | | |
| CAB40718.1 | LQESGGGLVQ | RTSNIYA | MGWFRQAP | VDGTS | TYYASSVRGRFTISRDDA | RADAEGWW | DYWGQG | Lama |
| | AGGSLRLSCAA | | GQEREAVA | | KNTLYLIMNSLKPEDTGL | | TQVTVSS | |
| | SG | | AIN | | YYCAT | | | |
| CAB40719.1 | LQESGGGVVLA | FTFEDYA | IGWFRQAP | GSDGR | TRYGKAVQGRVSVSKDN | SPWGGEVYGTPP | IYWGKGT | Lama |
| | GGSLRLSCTVS | | GKERERVG | | AKSTIYLQMNNLDDNDTA | CLHQGM | QVTVSA | |
| | G | | CIS | | VYYCAA | | | |
| CAB40722.1 | LQESGGGSVQ | FKLGYYA | IGWFRQAP | NDGS | TYYADSMKGRFTISRNNA | SEFGTAQPLGIRE | DYWGRGT | Lama |
| | PGGSLRLSCAA | | GKEREGVS | | KNMVYLEMNSLKPEDTA | Y | QVTVSS | |
| | AG | | CIT | | VYYCAM | | | |
| CAB40724.1 | LQESGGGLVQ | LRFSDYV | MGWFRQAP | WDGG | TNYADSVKGRFTMSKDN | TRGRSTRLVLPSL | ESWGQGT | Lama |
| | AGGSLRLSCAA | | GKEREFVA | M | AKNTVYLQMNSLRPEDT | V | QVTVSS | |
| | SG | | VIT | | AVYYCAA | | | |
| CAB40725.1 | LQESGGGLVQ | LLFSSYD | MGWFRQFP | ASGRR | TDYVDSVKGRFTVSRDV | GKSAYRGGAHYT | DYWGQG | Lama |
| | AGGSLALSCAA | | GKEREFVA | | AKNTVFLQMNDLKPEDTA | TGAY | TQVTVSS | |
| | SG | | AIS | | VYYCAG | | | |
| CAB40726.1 | LQESGGGLVQ | IAFQGYA | IGWFRQAP | NGEGN | KYYEDSVKGRFTISRDNT | ALSEVWRGSENL | DYWGQG | Lama |
| | PGGSLRLSCEA | | GKERECVS | | KKTVYLEMNSLKPEDTAV | REGY | TEVTVSS | |
| | SG | | YIE | | YYCAA | | | |
| CAB40727.1 | LQESGGGLVQ | LSFSNYA | MGWFRQG | RSGGG | IYYADSVLGRSTISRDNAK | GNIYPSSNYDLNN | GYWGQG | Lama |
| | PGGSLTLSCSA | | PGKEREFV | | HTVYLQMNSLKPDDTAV | SRMY | TQVTVSS | |
| | SG | | AAIS | | YYCAG | | | |
| CAB40728.1 | LQESGGGLVQ | LPVDYYA | IGWFRQAP | NNDGR | TDYADSVKGRFTISRDNA | GRVPHTMMVLTT | DYWGQG | Lama |
| | PGGSLKLSCAA | | GKEREWVS | | KNTVYLQLNSLKPEDTAV | TAYY | TQVTVSS | |
| | TG | | FIS | | YYCAL | | | |
| CAB40729.1 | LQESGGGLVQ | LIIKDTT | AGWFRQAP | WSNEN | TAYADSVKGRFTISRDNA | RPERTYVITPFSD | SYWGQGT | Lama |
| | AGDSLRLSCAA | | GKNREFVA | | KNTVALQMNGLKPEDTA | DY | QVTVSS | |
| | SG | | GIN | | VYYCTA | | | |
| CAB40730.1 | LQESGGGLVQ | RTFS | MGWFRQAP | WSDSS | TYYTDSVRGRFTISRDNA | QGSLYDDYDGLPI | DYWGQG | Lama |
| | AGGSLRLSCAA | | GKEREFVA | | KNTVYLQMDSLKPVDTAI | KY | TQVTVSS | |
| | SG | | AIN | | YYCAA | | | |
| CAB40731.1 | LQESGGGLVQ | SISSIDY | MMWYRQA | SDGS | TNYADSVKGRFTISRDGA | NVASGLKIL | QYWGQG | Lama |
| | AGGSLTLSCAS | | PGKERDMV | | RDTLYLQMNNLSPEDTAV | | TQVTVSS | |
| | SR | | ARID | | YSCVA | | | |
| CAC07788.1 | QVQLQESGGG | FAFNLYW | MYWFRQAP | PGNGIT | TFYADSVKGRFAISRDNA | DPSYQLADFLTSL | DYSGQGT | Lama |
| | LVQPGGSLRLS | | GKGLEWVS | FN | KNTLYLEMNSLQPEDTAV | PN | QVTVSS | |
| | CAASG | | SAS | | YYCAA | | | |
| CAC07789.1 | QVQLQESGGG | LSFSEFV | MTWFRQAP | WMDD | TYYGSSVKGRFFISKDNA | SRDYYGHNANQY | RYWGQG | Lama |
| | LVQAGESLRLS | | GKEREFVA | R | KNTVYLQMNGLKPEDTA | | TQVTVSS | |
| | CVASG | | AIN | | VYYCAA | | | |
| CAD13187.1 | QVQLVESGGG | YAYSSNC | MGWFRQAP | SGGGS | TYYADSVKGRFTISQDNA | HWKAYYNCGMWI | NNWGQG | Lama |
| | SVQAGGSLRLS | | GKEREGVA | | KNTVYLQMNSLKPEDTA | AY | TQVTVSS | |
| | CAASG | | GIY | | MYYCAV | | | |
| CAD22457.1 | LQESGGGLVQ | RTFSSYA | MGWFRQAP | WSGTI | TSYADSVKGRFTISRDNA | ALGYGSRHLDAY | AYWGLGT | Lama |
| | AGGSLRLSCAA | | GKEREFVA | | KNMVYLQMNSLKPEDTA | | QVT | |
| | SG | | AIS | | VYYCAA | | | |
| CAD22458.1 | LQESGGGLVQ | SYFSINR | MGWYRQA | SGGS | TNYADSVKGRFTISRDNA | NAYHSGNLY | TYWGQGT | Lama |
| | AGGSLRLSCAA | | PGNQRELV | | KNTVYLQMNSLKPEDTAV | | QVT | |
| | SE | | ASIS | | YLCNV | | | |
| CAD22459.1 | LQESGGGLVQ | STSSRINA | MGWYRQA | DGGS | TTYADSVKGRFTISRDNA | RVRFFTTYV | NNWGQG | Lama |
| | AGGSLTLSCAA | | PGKQRELV | | KNTMSLLMNSLQPEDTA | | TQVT | |
| | SG | | ASIN | | VYYCNA | | | |
| CAD22461.1 | LQESGGGLVQ | STFSINA | MGWYRQA | SGGG | TNYADSVKGRFTISRDNA | VQQSWTRGGLRS | DYWGQG | Lama |
| | AGGSLRLSCAA | | PGKQRELV | | KNTLYLQMNSLKPEDTAV | Y | TQVT | |
| | SV | | ADIS | | YYCNA | | | |
| CAD22462.1 | LQESGGGLVQ | STFSINA | MGWYRQA | YGGS | TKYADSVKGRFTISRDNA | EGSSWKKF | DYWGQG | Lama |
| | AGGSLRLSCAA | | PGKQRELV | | KNTVYLQMNSLKPEDTAV | | TQVT | |
| | SG | | ALIS | | YYCNA | | | |
| CAD22464.1 | LQESGGGLVQ | SIVSFNA | MAWYRQAP | NVG | TNYAGSVKGRFTISRDTA | DS | IGWGQGT | Lama |
| | PGGSLRLSCQA | | GKQRELVA | | KNTVYLQMNSLKPEDTAV | | QVT | |
| | ST | | LIT | | YYCNA | | | |
| CAD22465.1 | LQQSGGGLVQ | SISRNG | MDWYRQVP | SRGN | TNYADSVKGRFTISRDLA | R | GYWGQG | Lama |
| | PGGSLKLSCAA | | GKEREWVA | | KNMVDLQMNSLKPEDTA | | TQVT | |
| | SG | | TIT | | VYYCRW | | | |
| CAD22466.1 | LQQSGGGLVQ | STAKINA | MGWYRQA | SGGF | TTFASSVKGRFTISRDNA | LAPS | SYWGQGT | Lama |
| | AGGSLRLSCVA | | PGKSREFV | | KNTMYLQMNDLKLDDTG | | QVT | |
| | SG | | AGIT | | VYSCNA | | | |
| CAD22468.1 | LQESGGGLVQ | RTFGIYQ | TAWFRQVP | GYGTS | TYYADSVKGRFTISRDNA | DQTSLQLTKVSAY | RYWGQG | Lama |
| | AGGSLRLSCAP | | GKEREFVA | | KNTVYLQMNSLKPEDTAV | | TQVT | |
| | ST | | TIS | | YYCAA | | | |
| CAD22469.1 | LQESGGGLVQ | STSTVNG | MGWYRQTP | TGGT | TTYADSVKGRFTISRDNA | KNTIPYK | EFWGAGV | Lama |
| | AGGSLRLSCVV | | GNQRELVA | | KNTVYLQMDSLKPEDTAV | | QVT | |
| | SG | | LQS | | YYCNY | | | |
| CAD22470.1 | LQQSGGGLVQ | GTFTMYA | IDWLRQAP | AGGGS | TFYAESVKGRFTISRDNS | RRGIATMAPTSY | ATWGQGT | Lama |
| | AGGSLRLSCAA | | GKEREFVG | | KNTVSLQMNNLKPEDTA | | QVT | |
| | SG | | SIS | | VYYCAA | | | |
| CAD22477.1 | LQESGGGLVQ | FAFSINA | MDWYRQVP | SGGS | TNYADAVKGRFTISRDNA | AYKHNR | DSWGQG | Lama |
| | TGGSLRLSCAA | | GKEREVVA | | KQLVFLQMNNLKPEDTAV | | TQVT | |
| | SG | | RIS | | YYCNS | | | |
| CAD22472.1 | LQESGGGLVQ | STFSINA | ARWYRQAP | IGGT | TNYADSVKGRFTISRDNA | PGPYSDYETMAY | DYWGQG | Lama |
| | AGGSLRLSCAA | | GKQRELVA | | KNTVYLQMNSLKPEDTAV | EY | TQVT | |
| | SG | | AIS | | YYCNA | | | |
| CAD22473.1 | LQESGGGLVQ | RTFSNYH | MGWFRQAA | GTGSI | THYADSVKGRFTISGDSA | TVLARRPAPEAY | DYWGQG | Lama |
| | TGGSLRLSCAA | | GKEREFVA | | KNTVYLQMNNLKPEDTA | | TQVT | |
| | SG | | HMT | | VYYCAA | | | |
| CAD22479.1 | LQQSGGGLVQ | RTFSDYI | MGWFRQAP | WTGAI | TYVVDAVKGRFSISRDNA | GRSGVTVRNS | ESWGQGT | Lama |
| | AGGSLRLSCTA | | GKERERVA | | KNTVYLQMNGVKPEDTA | | QVT | |
| | SG | | TIN | | VYYCAA | | | |
| CAD22476.1 | LQESGGGLVQ | STFSINA | IRWYRQAP | IGGT | TNYADSVKGRFTISRDNA | PGPYSDYETMGY | DYWGQG | Lama |
| | AGGSLRLSCAA | | GKQRELVA | | KNTVYLQMNSLKPEDTAV | EY | TQVT | |
| | SG | | AIS | | YYCNA | | | |
| CAD22482.1 | LQESGGGLVQ | RTFSNYT | MGWFRQAP | GSGKL | AYVADSVKGRFIISRDNA | TILARRPSPGAY | DYWGQG | Lama |
| | AGDSLRLSCVA | | GKEREFVA | | KNTVYLQMNNLKPEDTAL | | AQVT | |
| | SG | | HIT | | YYCAA | | | |
| CAD22480.1 | LQQSGGGLVQ | RAFSSYH | MGWFRQAA | GTGGS | THYADSVKGRFTISRDNA | TILAQRPTPGAY | EYWGPGT | Lama |
| | AGGSLRLSCAA | | GKEREFVA | | KNTVYLQMNNLKPEDTA | | QVT | |
| | SG | | HMT | | VYYCAA | | | |
| CAE84869.1 | DVQLQASGGG | PIFDKNL | MGWGRQA | GSGG | TNYASSVEGRFTISRDNA | AF | AIWGQGT | Lama |
| | VVQPGGSLRLS | | PGKQREYV | | KKTVYLQMNDLKPEDTAV | | QVTVSS | |
| | CAAHD | | ATIS | | YYCNS | | | |
| CAF32436.1 | LQESGGGLVQ | RSDSTDT | MTWFRQAP | WIAN | TYYADSVKGRFTASRDYT | ADHGSRWWRQV | DYWGQG | Lama |
| | AGGSLRLSCAA | | GKEREFVA | | KNTVYLQMNNLTPEDTAV | ADITTY | TQVT | |
| | SG | | SVS | | YYCAA | | | |
| CAF32424.1 | LQESGGGSVQ | RTASNWA | THWFRQAP | SSGGY | TYYADSVKGRFTISRDNT | DSVNRAIMLGTSL | DYWGQG | Lama |
| | AGGSLRLSCAA | | GKEREFVA | | KSTVYLQMNSLKPEDTAV | | TQVT | |
| | SG | | AIS | | YYCAA | | | |
| CAF32421.1 | LQQSGGGLVQ | RTASSWA | IHWFRQAP | SSGEY | TYYADSVKGRFTISRDNT | DSSYRAIMLGRSL | DYWGQG | Lama |
| | TGGSLRLSCAA | | GKEREFVA | | KTTVYLQMDSLKPEDTAV | | TQVT | |
| | SG | | AIS | | YYCAA | | | |
| CAF32422.1 | LQQSGGGSVQ | RTASSWA | IHWFRQAP | SSGDY | TYYADSVKGRFTISRDNT | DSNYRAILLGTSL | NYWGQG | Lama |
| | AGGSLRLSCAA | | GKEREFVA | | KVTVYLQMNSLKPEDTAV | | TQVT | |
| | SG | | AIS | | YYCAA | | | |
| CAF32423.1 | LQQSGGGLVQ | RSFRTYA | MGWFRQAP | WSDSG | TYYADSVKGRFTISRDNA | DPSYARIVGSGRY | EYWGQGT | Lama |
| | NGDSLRLSCAA | | GKEREIVGA | | KNTLYLQMDSLKPEDTAV | II | QVT | |
| | SG | | LS | | YYCAA | | | |
| CAF32427.1 | LQESGGGLVQ | SIFSINA | AGWYRQAP | STGS | TNYADSVKGRFTISRDNA | DGIAVLAGRTDQY | DFWGQGT | Lama |
| | PGGSLRLSCAA | | GKQRELVA | | KNTVYLQMNSLKPEDTAV | | QVT | |
| | SG | | YMS | | YYCNA | | | |
| CAF32439.1 | LQESGGGLVQ | NISSVSF | MAWYRQTP | SGGY | PVYTDSVKGRFTISRDNA | AARTWFYNY | NYWGQG | Lama |
| | PGGSLRLSCAA | | GTQRELVA | | KNTLYLQMNGLKPEDTAV | | TQVT | |
| | SG | | QIP | | YYCYA | | | |
| CAF32430.1 | LQESGGGLVQ | SVASINV | MGWFRQVP | WSSGN | TYYHHFVEGRFTVSRDR | DTGLQYFSYAYS | VYWGQGT | Lama |
| | AGGSLRLSCAA | | GREREFVG | | AKNTVYLQMNSLKSEDTA | RRPSDY | QVT | |
| | SG | | AIG | | IYYCAA | | | |
| CAF32431.1 | LQESGGGLVQ | SVASINV | MGWFRQVP | LSGGN | TYYHHSVKGRFTVSRDR | DTGLAYFSYEYSR | GYWGQG | Lama |
| | AGGSLRLSCAA | | GREREFVG | | AKNTVYLQMDSLKSEDTA | RPSDY | TQVT | |
| | SR | | AIG | | IYYCAA | | | |
| CAF32432.1 | LQESGGGLVQ | RTDT | MTWFRQAP | WIDN | TNYADSVKGRFTVSRDY | ADHGSRWWRQM | DYWGQG | Lama |
| | AGGSLRLSCAA | | GKEREFVA | | TKNTMYLQMNNLAPEDT | ADATTY | TQVT | |
| | SG | | SIS | | AVYYCAA | | | |
| CAF32437.1 | LQQSGGGSVQ | RTASNWP | IHWFRQAP | WSGGY | TYYADSVKGRFTISRDNT | DSVNRAILLGTSL | DYWGQG | Lama |
| | AGGSLRLSCAA | | GKEREFVA | | KSTVYLQMNSLKPEDTAV | | TQVT | |
| | SG | | AIS | | YYCAA | | | |
| CAF32438.1 | LQQSGGGLVQ | SIFSINS | MGWYRQA | SGGR | INYADSVKGRFTISRDNA | DIIATLVGRKDSY | DFWGQGT | Lama |
| | PGGSLRLSCAA | | PGKQRELV | | KNTVYLQMNSLKPEDTAV | | QVT | |
| | SG | | AYIT | | YYCNA | | | |
| CAF32447.1 | LQESGGGLVQ | PIFSPNT | MAWFRQAP | YGGG | TTYVDSVKGRFTISRDNT | WSQWQSE | TYWGQGT | Lama |
| | PGGSLRLSCTA | | GKQRELVA | | KKMMSLQMNSLKPEDTA | | QVT | |
| | SG | | TIT | | VYYCAL | | | |
| CAF32443.1 | LQQSGGGSVQ | PIISPNT | MGWFRQAP | YNGG | TTYADSVKGRFTISRDNT | WSQWESE | TYWGQGT | Lama |
| | PGGSLRLSCAA | | GKQRELVA | | KKMMSLQMNSLNPDDTA | | QVT | |
| | SG | | TIT | | VYYCTL | | | |
| CAF32445.1 | LQESGGGLVQ | PITSPNT | MGWFRQAP | YGGG | TTYADSVKGRFTISRDNA | WNQWESE | SYWGQGT | Lama |
| | PGGSLRLSCVA | | GKQRELVA | | KKTMSLQMNSLKPEDTA | | QVT | |
| | SG | | TIT | | VYYCNL | | | |
| CAH18009.1 | LQESGGGLVQ | RTFSSYH | MGWFRQAA | SDYN | TYYADSVKGRFTISRDNA | RTVFSGPQYYTR | GYWGQG | Lama |
| | AGGSLRLSCEA | | GKEREFVA | | KNTVYLQTNSLKPEDTAV | AHEY | TQVT | |
| | SG | | TIS | | YYCAS | | | |
| CAH18015.1 | LQESGGRLVEA | GTFSWYA | MGWFRQAP | RGGGS | TYYADSVKGRFTISRDNA | GRGSPSDTGRPD | DYWGQG | Lama |
| | GGSLRLSCAVS | | GKEREFVA | | KNTVYLQMNSLKPEDTAV | EY | TQVT | |
| | G | | TVS | | YYCAA | | | |
| CAH18018.1 | LQESGGRLVEA | GTFSWYA | MGWFRQAP | RGGGS | IYYADSVKGRFTISRDNA | GRGAPSDTGRAD | DFWGQGT | Lama |
| | GGSLRLSCAAS | | GKEREFVA | | KNTVYLQMNSLKPEDTAV | EY | QVT | |
| | G | | TVS | | YYCAA | | | |
| 1hcv_A | DVQLQESGGG | RTGSTYD | MGWFRQAP | WDSAR | TYYASSVRGRFTISRDNA | GEGGTW | DSWGQG | Lama |
| | LVQAGGSLRLS | | GKERESVA | | KKTVYLQMNSLKPEDTAV | | TQVTVSS | |
| | CAASG | | AIN | | YTCGA | | | |
| 1qd0_A | QVQLQESGGG | RAASGHG | MGWFRQVP | WSGKE | TWYKDSVKGRFTISRDNA | RPVRVADISLPVG | DYWGQG | Lama |
| | LVQAGGSLRLS | HYG | GKEREFVA | | KTTVYLQMNSLKGEDTAV | F | TQVTVSS | |
| | CAASG | | AIR | | YYCAA | | | |
| 1shm_E | DVQLQESGGG | ATGSTYD | MGWFRQAP | WGSAG | TYYASSVRGRFTISRDNA | GRIGRSVFNLRRE | TWWGQG | Lama |
| | LVQAGGSLRLS | | GKERESVA | | KKTVYLQMNSLKPEDTAV | SWV | TQVTVSS | |
| | CAASG | | AIN | | YTCGA | | | |
| 1sjx_A | QVQLQESGGG | NIFRIND | MGWYRQA | SGGS | TKYADSVKGRFTISKDNA | EDRHRIGTV | GYWGQG | Lama |
| | LVQAGGSLRLS | | PGTQRELV | | KNTVYLQMNSLKPEDTAV | | TQVTVSS | |
| | CQASG | | AAIT | | YYCAA | | | |
| 1u0q_B | EVQLQESGGG | RTFSTYA | VGWFRQAP | TRGGR | TYYADSVKGRFTIAIDNAK | RMPYSGDYRSSG | DYWGQG | Lama |
| | LVQAGGSLRLS | | GKEREFVG | | NTVYLQMNSLKLDDTAVY | TY | TQVTVSS | |
| | CAASG | | YFG | | YCAV | | | |
| 2x1o_B | QVQLQESGGG | RNLRMYR | MGWFRQAP | WSSDT | IYYADSVKGRFIISRDNAK | GAGWAGTMTDY | NYWGQG | Lama |
| | LVQAGGSLRLS | | GKEREFVG | | NTVYLQMNSLKPEDTAVY | | TQVTVSS | |
| | CAAAG | | TMV | | YCAA | | | |
| 2x1p_D | QVQLQESGGG | RTFTSFA | MGWFRQAP | RSGTL | TRYADSAKGRFTISVDNA | DLHRPYGPGTQR | DSWGQG | Lama |
| | LVQAGGSLRLS | | GKEREFVA | | KNTVSLQMDNLNPDDTA | SDEY | TQVTVSS | |
| | CAASG | | SIS | | VYYCAA | | | |
| 2x1q_B | QVQLQESGGG | RTFSNYR | MGWFRQAP | QSGAA | TAYADSVKGRFTFSRDN | SSRVFYTEVLQTT | DYWGQG | Lama |
| | LVQPGGSLRLS | | GKEREFVA | | AKNLLYLEMLSLEPEDTA | TGY | TQVTVSS | |
| | CAASG | | TIS | | VYYCAA | | | |
| 2xa3_A | AVQLQESGGG | RTSSSHD | MGWFRQAP | WSGGT | TNYVDSVKGRFDISKDNA | KWRPLRYSDNPS | NYWGQG | Lama |
| | LVQAGGSLRLS | | GKEREFVA | | KNAVYLQMNSLKPEDTA | NSDY | TQVTVSS | |
| | CTVSA | | AIS | | VYYCAA | | | |
| 3ezj_H | QVQLQESGGG | SIFSINS | MDWDRQA | SGGS | TNYADSVKGRFTISRDNA | NVKTWAGMTR | DYWGQG | Lama |
| | LVQAGGSLRLS | | PGKQRELV | | KNTVYLQMNSLKPEDTAV | | TQVTVSS | |
| | CAASG | | ATIT | | YYCNA | | | |
| 3k7u_A | QVQLQESGGG | RTFSNNA | MGWFRQAP | WTGGL | LFYADSVNGRFTISRDNA | RPQGDYVTAHY | DYWGQG | Lama |
| | LVQAGGSLTLS | | GKEREFVA | | KRTVTLQMNSLKPEDTAV | | TQVTVSS | |
| | CAASG | | AIS | | YYCAA | | | |
| 3k80_B | QVQLQESGGG | RAFSSYG | MGWFRQAP | RSGGL | TQYAESLKGRFAISRDNA | DLYGLGSHMENE | DSWGQG | Lama |
| | LVQAGDSLRLS | | GKERAFVA | | KNTVYLQMGSLKPEDTA | Y | TQVTVSS | |
| | CVASG | | AIS | | VYYCAG | | | |
| 3k81_B | EVQPQESGGG | ITFASEA | WGWYRRA | NEGR | TNYVDSVKGRFTVSRDN | NLQTGTLSGAR | LYWGQGT | Lama |
| | LAQAGGSLRLS | | PGKQRELIA | | AKNVMYLQMNSLKPEDT | | QVTVSS | |
| | CVVSG | | AIN | | AVYYCNA | | | |
| 3rjq_B | AVQLQESGGG | RISSSYD | MGWFRQAP | WSGGT | TDYADSVKGRFAISKDNA | KWRPLRYSDYPS | DYYDWG | Lama |
| | LVQAGGSLRLS | | GKEREFVA | | KNAVYLQMNSLKPEDTA | NS | QGTQVTV | |
| | CTASG | | AIS | | VYYCAA | | | |
| 3stb_B | QVQLQESGGG | RTLSSYA | MGWFRQAP | RSGS | TFYADAVKGRFTISRDNA | DRFSPVVPGPIPV | DSWGQG | Lama |
| | LVQAGGSLRLS | | GKEREFVA | | KNTVYLQMNSLKPEDTAA | NTV | TQVTVSS | |
| | CAASG | | AIN | | YYCAA | | | |
| 4aq1_D | QVQLQESGGG | RTSSAYA | MGWFRQAP | SKGGS | TYYGASMKGRFTISRDNA | SDKYNFDTSHAG | GYWGQG | Lama |
| | LVQAGGSLRLS | | GKEREFVA | | KNTVYLQMNGLAPEDTA | Y | TQVTVSS | |
| | CAASG | | GIS | | VYYCAA | | | |
| 4y8d_D | QVQLQESGGG | YTSR | MGWFRQAP | RQSNL | SYYSDSVRGRFTISQDNA | TTDCAAFVERATA | ITAGQGT | Lama |
| | SVQAGGSLRLS | | GAEREGVA | | KTTAFLLMSSLKPEDTAIY | | QVTVSS | |
| | CGASE | | CIH | | YCAT | | | |
| 4cdg_D | QVQLQESGGG | IWFSINN | MAWYRQTP | SAGT | TNYVDSVKGRFTISRDDA | VADYDMGFQ | SFWGRGT | Lama |
| | LVQAGGSLRLS | | GKQRERIAI | | KNTMYLQMNSLIPEDTAV | | QVTVSS | |
| | CAASG | | IT | | YYCNL | | | |
| 4dka_B | QVQLQESGGG | RTSSLYS | MGWFRQAP | RNGAN | TYYTDSVKGRFTISRDNA | DRFPTMEVVTIMT | DYWGQG | Lama |
| | LVQAGGSLRLS | | GKEREFVA | | KNTVELQMNSLKPEDTAV | NEY | TQVTVSS | |
| | CAASG | | AIS | | YYCAA | | | |
| 4eig_B | QVQLQESGGG | IIFSVYK | MTWYRQAP | TNNN | TMTVDSVKGRFTISRDNV | NRGLAGP | AYWGQGT | Lama |
| | LVQAGGSLRLS | | GKERELVAL | | QNTVYLEMNNLKPEDTA | | QVTVSS | |
| | CKASG | | IT | | VYYCNA | | | |
| 4ej1_D | QVQLQESGGG | RTFSSYA | MGWFRQTP | WGGST | TLYADSVKGRFTMSRDN | DGSQYRSTYSFR | GSWGQG | Lama |
| | LVQAGGSLRLS | | GKEREFVA | | AKNTVYLQMNSLKPEDTA | DKPDY | TQVTVSS | |
| | CTASG | | AIT | | VYYCAA | | | |
| 4fhb_D | QVQLQESGGG | RTFSSYA | MGWFRQAP | WSGSN | TYYADSAKGRFTISRDNA | PGRPHGSSWSLN | DYWGQG | Lama |
| | LVQAGGSLRLS | | GKERDFVA | | KNTVYLQMNSLKPEDTAI | KKGQGY | TQVTVSS | |
| | CEASG | | VIS | | YYCAA | | | |
| 4gft_B | EVQLQESGGG | ERAFSNYA | MGWFRQAP | GSGRS | QYYADSVKGRFTISRDNA | RVVPVFSDSTKG | VYWGQGT | Lama |
| | TVQPGGSLKLS | | GQEREFVA | | MNAVYLQMNSVKAEDTA | Y | QVTVSS | |
| | CSAAP | | GIT | | VYYCAA | | | |
| 4grw_J | EVQLVESGGGL | FTLDYLA | IGWFRQAP | SSGQY | TYYADSVKGRFTISRDNA | DPECYRVRGYYN | DYWGQG | Lama |
| | VQPGGSLRLSC | | GKEREGVS | | ESTVYLQMNSLKPEDTAV | GEY | TQVTVSS | |
| | AASG | | CVS | | YYCAT | | | |
| 5mzv_D | EVQLVESGGGL | RTFSWSA | VGWFRQAP | WSGGS | PYYADSVKDRFTISRDNA | TSLFPTSRGSHY | DTWGQGT | Lama |
| | VQAGGSLRLSC | | GKEREFVA | | KNTVYLQMNSLRPEDTA | | QVTVSS | |
| | AASG | | AIR | | VYLCGE | | | |
| 4hem_G | QVQLVESGGG | STFSNYA | MGWFRQAP | QTGSH | TYYRNSVKGRFTISRDNA | GDNYYYTRTYEY | DYWGQG | Lama |
| | LVQAGGSLRLS | | GPEREFVA | | KNTVYLQMNNMKPEDTA | | TQVTVSS | |
| | CAASE | | TIS | | VYYCAA | | | |
| 4i13_B | QVQLQESGGG | RLTVDYA | IGWFRQAP | WGGGL | TVYGESVEGRFTISRDIAK | SRISYRVWNTIPY | TLWGRGT | Lama |
| | LVQAGASLRLS | | GKEREFVA | | NTMNLQMNVLRPEDTAN | NKL | QVTVSS | |
| | CAASE | | AIS | | YYCAA | | | |
| 4i1n_B | QVQLQESGGG | RLTVDYA | IGWFRQAP | WGGGL | TVYGESVEGRFTISRDIAK | SRISYAVWNTIPY | TLWGRGT | Lama |
| | LVQAGASLRLS | | GKEREFVA | | NTMNLQMNVLRPEDTAN | NKL | QVTVSS | |
| | CAASE | | AIS | | YYCAA | | | |
| 4idl_A | KVQLQQSGGG | NTASIRA | MGWYRRAP | TTGT | ADYGDFVKGRFTISRDNA | DGRRFDGARWR | ESWGQGT | Lama |
| | AVQTGGSLKLT | | GKQREWVA | | NNAATLQMDSLKPEDTA | EY | QVTISS | |
| | CLASG | | SLT | | VYYCNA | | | |
| 4n9o_B | AAQLQESGGG | RTFSSYN | MGWFRQAP | SSGDK | SDYTDSVKGRFTISRDNA | GLGIYIIRARGGY | DHWGQG | Lama |
| | LVQPGGSLRLS | | GKGREFVA | | KNTMYLQMNNLKPEDTA | | TQVTVSS | |
| | CAASG | | SIT | | TYYCAR | | | |
| 4krm_L | QVKLEESGGG | RTSRSYG | MGWFRQAP | WRGDS | TGYADSVKGRFTISRDNA | AAGSAWYGTLYE | DYWGQG | Lama |
| | SVQTGGSLRLT | | GKEREFVS | | KNTVDLQMNSLKPEDTAI | Y | TQVTVSS | |
| | CAASG | | GIS | | YYCAA | | | |
| 4kro_B | QVQLQESGGG | RTFSSYA | MGWFRQAP | WSGGY | TYYTDSVKGRFTISRDNA | TYLSSDYSRYALP | DYWGQG | Lama |
| | LVQPGGSLRLS | | GKQREFVA | | KTTVYLQMNSLKPEDTAV | QRPLDY | TQVTVSS | |
| | CAASG | | AIR | | YYCAA | | | |
| 4krp_B | EVQLVESGGGL | RTFSSYA | MGWFRQAP | WSSGS | TYYADSVKGRFTISRDNA | GYQINSGNYNFK | DYWGQG | Lama |
| | VQAGGSLRLSC | | GKEREFVV | | KNTMYLQMNSLKPEDTA | DYEY | TQVTVSS | |
| | AASG | | AIN | | VYYCAA | | | |
| 4laj_M | EVQLVESGGGL | FTLDYYS | IGWFRQAP | DSDGR | TYYADSVKGRFTISRDNA | DCTVDPSLLYVM | DYYGKGT | Lama |
| | VQPGGSLRLSC | | GKEREGVS | | KNTVYLQMNSLKPEDTAV | | QVTVSS | |
| | AASG | | CIS | | YYCAT | | | |
| 4m3k_B | QVQLQESGGG | SISSITT | MGWYRQD | SVGD | TTYAGSVKGRFTISRDNA | FMSTNSGRTG | SFWGQGT | Lama |
| | LVQPGGSLRLS | | PGKGRELV | | KNTVYLEMSSLKPEDTAV | | QVTVSS | |
| | CAASG | | ALIN | | YYCNA | | | |
| 4n1h_D | QVQLQESGGG | RTFSSYA | MGWFRQAP | RSGGD | TKYADSVKGRFAISRDND | TTYASLSDTYIGE | DDWGQG | Lama |
| | LVQAGASLKLS | | GKEREFVA | | KNTVWLRMNSLKPEDTA | HIY | TQVTVSS | |
| | CAASG | | AIS | | VYYCAA | | | |
| 4nc1_D | QVQLVESGGG | RTFSMDP | MAWFRQPP | STGRT | TYYADSVKGRFTISRDNA | APYGANWYRDEY | AYWGQGT | Lama |
| | LAQAGGSLRLS | | GKEREFVA | | KNTVYLQMNSLKPEDTAV | | QVTVSS | |
| | CAASG | | AGS | | YYCAA | | | |
| 4nc1_F | QVKLEESGGGL | RTFSRYP | VAWFRQAP | STGTS | TYYADSVKGRFTISRDNA | NSQRTRLQDPNE | DYWGQG | Lama |
| | VQAGGSLRLSC | | GAEREFVA | | KVTVYLQMNNLKREDTA | Y | TQVTVSS | |
| | AASE | | VIS | | VYFCAV | | | |
| 4nc2_B | QVQLVESGGG | LTFSRYV | MGWFRQAP | WGGT | PNYADSVKGRFTISRDNS | GLGWDSRYSQSY | NYWGQG | Lama |
| | LVQAGGSLRLS | | GKEREFVA | | KNTQYLQMNSLKPEDTA | | TQVTVSS | |
| | CAASG | | AIT | | VYYCAA | | | |
| 4ocn_F | QVQLQESGGG | RTFSSTV | MAWFRQAP | WSGGN | TYYADSVKGRFTISRDNA | GTYYGTLSYKY | DFWGRGT | Lama |
| | LVPAGGSLRLS | | GKEREFVA | | RNTVYLQMNSLKPEDTA | | QVTVSS | |
| | CVDSG | | TIR | | VYYCAG | | | |
| 4orz_C | EVQLVESGGGL | FTFGTSN | MAWLRQAP | ISGY | TDYADSVKDRFTISRDNA | RVGSEY | DLWGQGT | Lama |
| | VQAGGSLRLFC | | GKRREWVA | | KNTVSLQMNSLKPEDTAI | | QVTVSS | |
| | AASG | | LIT | | YFCAR | | | |
| 4p2c_K | QVQLQESGGG | SIFRLST | MGWYRQA | SYGD | TNYRDSVKGRFTISRDNA | NIEAGTYYGPGR | DYWGQG | Lama |
| | LVQAGGSLRLS | | PGKQREFV | | KNTVYLQMNSLKPEDTAV | | TQVTVSS | |
| | CAVSG | | ASIT | | YYCNA | | | |
| 4pir_J | DVQLVESGGG | SLFSILR | MDWYRQAP | RDAAG | TNYADSVKGRFTISRDSA | DARTITGRA | DYWGQG | Lama |
| | LVQPGGSLRLS | | GKERELVA | YADS | KNTVYLQMNSLKPEDTAV | | TQVTVSS | |
| | CAYSG | | GIT | | YYCNA | | | |
| 4poy_A | QVQLVESGGG | YPHPYLH | MGWFRQAP | SGGGG | TLYADSVKGRFTISRDKG | GGYQLRDRTY | GHWGQG | Lama |
| | LVQAGGSLRLS | | GKEREGVA | | KNTVYLQMDSLKPEDTAT | | TQVTVSS | |
| | CAASG | | AMD | | YYCAA | | | |
| 4qlr_B | DVQLVESGGG | RTFSDYA | LGWFRQAP | WSGGS | TYYADSVQGRFTISRDND | DKYYTGPGGESV | DYWGRGT | Lama |
| | LVHPGGSLRLS | | GKDREFVA | | KNSVYLQMNSLKPEDTA | Y | QVTVSS | |
| | CAASG | | AIS | | VYYCAA | | | |
| 4qo1_A | QVQLQESGGG | RTFSTYA | MGWFRQAP | WSGTT | TYYAESVKDRTTISRDNA | HPQRGWGSTLG | TYWGQGT | Lama |
| | LVQAGGSLRLS | | GREREFLA | | KNTVYLEMNNLNADDTGI | W | QVTVSS | |
| | CAASE | | QIN | | YFCAA | | | |
| 4w81_A | EVQLVESGGGL | RTFSRAV | MGWFRQAP | AAPGT | AFYADSVRGRFSISADSA | DLKMQVAAYMNQ | DYWGQG | Lama |
| | VQAGDSLRLSA | | GKEREFVA | AYY | KNTVYLQMNSLKPEDTAV | RSV | TQVTVSS | |
| | TASG | | AIS | | YYVAA | | | |
| 4w6w_B | QVQLQESGGG | YTSGRDS | MGWFRQAP | TSGI | VNYADSVKGRFTISQDSA | GPFVYGRGCLGQ | SYWGQGT | Lama |
| | SVQAGGSLRLS | | GKEREGVA | | KKTLYLEMNSLKPEDTAL | AFY | QVTVSS | |
| | CAASG | | CID | | YSCAT | | | |
| 4w6y_B | QVQLQESGGG | YTYSSNC | MAWFRQVP | TRGGI | TYYADSVKGRFTISRDNA | VREATYSDNRCS | DYWGQG | Lama |
| | SVQAGGSLRLS | | GKEREGVA | | KNTVSLQMNSLKPEDTAT | VRSYTY | TQVTVSS | |
| | CAASG | | SIN | | YYCAA | | | |
| 5m95_D | QVQLQESGGG | SIFSIDT | ANWYRQPP | RDGN | ANYADSVKGRFTISRDRA | AIRTTVRTSAQ | EYWGQGT | Lama |
| | LVQAGGSLRLS | | GMQRELVA | | RNTVYLQMNSLKPEDTG | | QVTVSS | |
| | CAASR | | TIT | | VYYCNA | | | |
| 5o04_D | QVQLQESGGG | SIFSIYA | MGWYRQA | SGGG | TNYADSVKGRFTISGDNA | EDYSAYAPPS | GSRGRGT | Vicugna |
| | LVQPGGSLRLS | | PGKQRELV | | KNTVYLQMNSLKPEDTAV | | QVTVSS | |
| | CAASG | | ASIS | | YYCKR | | | |
| 4zg1_F | QVQLQESGGG | STFGIRT | MGWYRQA | SGGS | TDYADSVKGRFTISRDNA | RVGITML | AHWGQG | Lama |
| | LVQAGGSLRLS | | PGKQRDLV | | KNTVYLQMDSLKPEDTAI | | TQVTVSS | |
| | CAASG | | AIIS | | YYCNA | | | |
| 5bop_C | QVQLQESGGG | GTFSTYG | MGWFRQAP | WTGAN | TYYADSVRGRFTISRDNA | RRWLGGSYFDPG | DFWGQGT | Lama |
| | LVQAGGSLRLS | | GKEREFVA | | KNTVYLEMNSLKPEDTAV | NY | QVTVSS | |
| | CAASG | | ASS | | YYCAA | | | |
| 5da4_C | QVQLQESGGG | RTFSSDV | MGWFRQAP | RSGGK | SYNADSVKGRFTISRDNA | GDTAITSWYGY | DYWGQG | Lama |
| | LVQAGGSLRLS | | GKEREFVA | | KNTVSLQMNSLKPEDTAV | | TQVTVSS | |
| | CAASG | | AVT | | YYCAA | | | |
| 5dfz_E | QVQLQESGGG | PFSPNS | MGWYRQA | IDGR | TNYQDSVKGRFTISRDYV | ETRGF | MHWGQG | Lama |
| | LVQPGGSLRLS | | PGKQRELV | | KNTAYLQMNNLKPDDTA | | TQVTVSS | |
| | CAPSG | | AVMT | | VYICNA | | | |
| 5f1k_D | DVQLQESGGG | RTFRNYP | MAWFRQAP | WVGAS | TLYADFAKGRFTISRDNA | GRGIVAGRIPAEY | ADWGQG | Lama |
| | LVQAGGSLRLS | | GKEREFVA | | KNTVYLQMNSLKPEDTAV | | TQVTVSS | |
| | CTGSG | | GIT | | YSCAA | | | |
| 5f1o_B | DVQLQESGGG | SREDNYA | MGWFRQAP | WSSGT | TRYLDTVKGRFTISRDNA | RYQPRYYDSGDM | DNWGQG | Lama |
| | LVQAGHSLRLS | | GKEREFVA | | KSTVYLQMNSLKPEDTAV | DGYEY | TQVTVSS | |
| | CVGSG | | AIS | | YYCAA | | | |
| 5f7n_D | QVQLQESGGG | SIFSGNV | MGWYRQA | PQGV | PNYADSVKGRFTISRDNA | LPNY | RSWGQG | Vicugna |
| | LVQPGGSLRLS | | PGKLREWV | | KNMLYLQMSSLKPEDTAL | | TQVTVSS | |
| | CAASG | | AAIT | | YYCNR | | | |
| 5f7l_D | QVQLQESGGG | SIYSLIA | MGWYRQA | SGST | TYYADSVKGRFTISRDNA | YSDRLTDCSNCE | DYWGQG | Lama |
| | LVQPGGSLRLS | | PGKEHELV | | KNTLYLQMNSLKPEDTA | A | TQVTVSH | |
| | CAASG | | ATIS | | MYYCAA | | | |
| 5g5x_B | QVQLQESGGG | SGFNNNA | MGWYRQA | SFGS | TNYADSVKGRFTISRDNA | GWGATPR | SYWGQGT | Lama |
| | LVQPGGSLRLS | | PGKQRELV | | KNTVYLQMNSLKPEDTAV | | QVTVSS | |
| | CAASG | | AAIT | | YYCTA | | | |
| 5hm1_C | DVQLQESGGG | NIVSIDA | AGWFRQAP | TGGA | TNYADSVKGRFTISRDNA | PMIYYGGRYS | DYWGQG | Lama |
| | LVQPGGSLRLS | | GKQREPVA | | KNTVYLQMNSLKPEDTAV | | TQVTVS | |
| | CAASG | | TIL | | YYCYA | | | |
| 5ip4_B | QVQLQESGGG | NIIGGNH | WAWYRQA | RYN | ANYADSVKGRFTISRDNA | EN | YYWGQGT | Lama |
| | LVQAGGSLRLS | | PGQQRDLV | | KNAAYLQMNSLKPEDTAI | | QVTVSS | |
| | CTASV | | ASLS | | YFCAL | | | |
| 5ja8_H | QVQLQESGGG | FTLDYYA | GWFRQAP | SSGGT | TNYADSVKGRFTVSRDN | DFACPLIREY | DYWGQG | Lama |
| | LVQPGGSLRLS | | GKEREGVS | | AKNTVYLQMNSLKPEDTA | | TQVTVSS | |
| | CAASG | | CIS | | VYYCVA | | | |
| 5ja9_B | QVQLQESGGG | FTFSNYA | MRWYRQAP | SVGGS | TNYADSVKGRFTISRDNG | RLSLIS | DSWGQG | Lama |
| | LVQPGGSLRLS | | GEEREFVA | | KNTLYLQMNSLKPEDTAV | | TQVTVSS | |
| | CAASG | | FIS | | YFCVA | | | |
| 5jqh_D | QVQLQESGGG | SIFSLND | MGWYRQA | SGGS | TKYADSVKGRFTISRDNA | KVAGTFSIY | DYWGQG | Lama |
| | LVQAGGSLRLS | | PGKLRELVA | | KNTVYLQMNSLKAEDTAV | | TQVTVSS | |
| | CAASG | | AIT | | YYCNA | | | |
| 5lwf_D | QVQLQESGGG | RTFSNDH | MGWFRKAP | PGTEK | TYYADSVKGRFAFSRDN | TPYYRGSYYAAS | TYWGQGT | Lama |
| | LVQAGGSLRLS | | GKEREFVA | | AKNTVYLQMNSLKPEDTA | TY | QVTVSS | |
| | CAASG | | AIT | | VYYCVA | | | |
| 5m2i_L | VQLVESGGGLV | RSLSNYY | MGWFRQAP | WRGYN | IYYKDSVKGRFTISRDDA | SILPLSDDPGWN | TYWGQGT | Lama |
| | QAGGSLSLSCS | | GKERELLG | | KNTIYLQMNRLKPEDTAV | | QVTVS | |
| | ASG | | NIS | | YYCAA | | | |
| 5m2m_N | QVQLQESGGG | RTFSDHSG | IGWFRQAP | WSSGN | TYYADSVKGRFAISRDIAK | RDGIPTSRSVESY | NYWGQG | Lama |
| | LVQPGGSLRLS | YTYT | GKEREFVA | | NTVDLTMNNLEPEDTAVY | | TQVTVSS | |
| | CAASG | | RIY | | YCAA | | | |
| 5m30_F | QVQLVESGGG | SISGIVV | MAWYRQAP | SGGT | TNYADSVKGRFTISKDNA | FFRRDYVGY | DYWGQG | Vicugna |
| | LVQAGGTLKLS | | GKQRELVA | | ENTLYLRMNSLKPEDTAV | | TQVTVSS | |
| | CAASG | | SIT | | YYCKA | | | |
| 5mp2_D | QVQLVESGGG | NTDSSYY | MGWFRQG | IRAGI | PYYTDSVKGRFTISQDNA | SVRTTIQPFKGNY | NYWGRGT | Lama |
| | SVQAGGSLRLS | | PGKEREGV | | KNTIYLQMNSLKPEDTAM | Y | QVTVSS | |
| | CAASG | | ASIY | | YFCAG | | | |
| 5mwn_N | QVQLVESGGG | IMLGYFT | MAWYRQAP | SGGS | ANYADAVKGRFTISRDNA | RIWRGTVYD | NISGPGT | Lama |
| | LVQPGGSLRLS | | GKQRELVA | | RSTVYLQMNSLKPEDTAV | | QVTVSS | |
| | CAASG | | TEI | | YYCDA | | | |
| 5o03_D | QVQLQESGGG | FTLGYYP | IGWFRQAP | GSGGS | ANYAASVKGRFTISRDNA | DLSSLTTVQAMC | DYWGLGT | Lama |
| | LVQPGGSLRLS | | GKGLEGVS | | KNTVYLQMNSLKPEDTAI | VIPRPGFSAKAY | QVTVSS | |
| | CAASG | | CIS | | YYCAA | | | |
| 5o2u_D | DVQLQESGGG | SISRFNA | MGWWRQA | KGFD | PVLADSVKGRFTISIDSAE | ALDT | AYWGQGT | Lama |
| | LVQAGGSLRLS | | PGKEREFV | | NTLALQMNRLKPEDTAVY | | QVTVSS | |
| | CAASG | | ARIV | | YCFA | | | |
| 5ojm_O | QVQLQESGGG | HTFNYPI | MGWFRQAP | WSGGS | TSYADSVKDRFTISRDNA | KGRYSGGLYYPT | DYWGQG | Lama |
| | LVQAGGSLRLS | | GKEREFVG | | KNTVYLEMNNLKPEDTAV | NY | TQVTVSS | |
| | CAASG | | AIS | | YYCAA | | | |
| 5ocl_G | QVQLQESGGG | RTWSIYG | MGWFRQAP | WRGG | THYADFVKGRFTISRDNV | NPNPSGSSVYRR | DYWGQG | Lama |
| | LVQAGDSLRLS | | GKEREFVA | N | KNTVYLQMNSLKPEDTAV | N | TQVTVSS | |
| | CAASG | | GIT | | YYCAA | | | |
| 2xxm_B | QVQLVESGGG | SFFMSNV | MAWYRQAP | GGDMS | TVYDDSVKGRFTITRDDD | SG | SSWGQGT | Vicugna |
| | LVQAGGSLRLS | | GKARELIAA | | KNILYLQMNDLKPEDTAM | | QVTVSS | |
| | CAASG | | IR | | YYCKA | | | |
| AHA34195.1 | QVQLVETGGG | GTLEHYA | IGWFRQAP | GEYGS | TVYVDSVKGRFTASRDN | GCYSWR | GPWGQG | Vicugna |
| | LVQPGGSLTLS | | GKEHEWLV | | AKNTVYLQLNSLKPDDTG | | TQVTVSS | |
| | CAGSG | | CNR | | IYYCVS | | | |
| ATL24999.1 | QVQLVESGGG | SIASIYR | TCWYRQGT | SGGN | TYYADSVKGRFTISRDNA | DEAGIGGEN | DYWGQG | Vicugna |
| | LVQPGGSLRLH | | GKQRELVA | | KNTIDLQMNSLKPKDTAV | | TQVTVSS | |
| | CAASG | | AIT | | YYCNA | | | |
| AHA34189.1 | QVQLVESGGG | GDFSRNA | MAWFRQAP | WTGSG | TYYLDSVKGRFTISRDNA | STVFAEITGLAGY | DYWGQG | Vicugna |
| | LVQAGGSLSLS | | GKEREFVA | | KNALYLQMNNLKPEDTAV | QSGSY | TQVTVSS | |
| | CAASG | | SIN | | YYCAR | | | |
| AHA34197.1 | QVQLVESGGG | SIVNFET | MGWYRQA | NEGS | SNYADSVKGRFTISGDNA | TFGSRWPYAHS | DHWGQG | Vicugna |
| | LVQAGGSLRLS | | PGKERELV | | KNTVSLQMNSLKPEDTAV | | TQVTVSS | |
| | CAASG | | ATIT | | YYCSA | | | |
| 4lhq_D | VQLVETGGGTV | GSFSRNA | MGWFRQAP | WSASS | TYYRDSVKGRFTVSRDN | SSVYAEMPYADS | NYWGQG | Vicugna |
| | QTGGSLRLSCS | | GKEREFVA | | AKNTVYLHLNSLKLEDTA | VKATSY | TQVTVSS | |
| | ASG | | AIN | | AYYCAG | | | |
| 4tvs_b | QVQLVESGGG | RTLSSYA | VGWFRQAP | RSGGS | THYADSVKGRFTISRDNA | TFTPDGSWYYTR | DYWGQG | Vicugna |
| | LVQAGGSLRLS | | GLEREFVAT | | KNTVYLQMNSLKPEDTAV | GSSY | TQVTVSS | |
| | CAASG | | IS | | YYCAA | | | |
| 4x7f_D | DVQLVESGGG | SILSFNH | MAWYRQG | REGS | TDYADSVKGRFTISRDNA | GIS | NPWGQG | Vicugna |
| | LVQPGGSLRLS | | PGEQRELV | | KNMVYLLMSNLRPEDTA | | TQVTVSS | |
| | CAASE | | AVIT | | VYYCNR | | | |
| 4xt1_C | QVQLVESGGG | SIFTIYA | MGWYRQA | FGGD | TNYADSVKGRFTISRDNA | EETIVEEA | DYWGQG | Vicugna |
| | LVRPGGSLRLS | | PGKQRELV | | KNAVYLQMNSLKPEDTA | | TQVTVSS | |
| | CAASG | | ARIT | | VYYCNA | | | |
| AHA34193.1 | QVQLVESGGG | RTFSRSS | MGWFRQAP | WADGT | TLYGDSVKGRFTVSRDN | NKFVRGLVAVRAI | DYWGQG | Vicugna |
| | LVQAGGSLRLS | | GKEREFVA | | VKNMVYLQMNNLKPEDT | DY | TQVTVSS | |
| | CAASG | | SIV | | ALYYCAD | | | |
| ATL24956.1 | QVQLAETGGG | TTFSKNA | MAWFRQAP | WNAVS | TNYADSVKGRFTVSRDN | SSIYSDISGAATV | NYWGQG | Vicugna |
| | LVQAGGSLRLS | | GKEREFVA | | AKNTVYLQMNSLKPEDTA | WATSY | TQVTVSS | |
| | CAASG | | GIN | | VYYCAG | | | |
| 5gxb_B | QVQLVESGGR | RTFTTYL | MGWFRQAP | WSGGS | TYYADSVKGRFTISRDNA | AARPSYSGDYGY | DYWGQG | Vicugna |
| | LVQAGDSLRLS | | GKEREFVA | | KNTVYLQMNSLKLEDTAV | TEALRY | TQVTVSS | |
| | CAASG | | AIR | | YYCAA | | | |
| 5hvh_C | QVQLQESGGG | SIFSPNA | MGWYRQA | NVG | STYADSVKGRFTVSRDN | WGQDGWLGQY | DYWGQG | Vicugna |
| | LVQAGGSLRLS | | PGKERELV | | AKNTVYLQMNSLKPEDTA | | TQVTVSS | |
| | CAASG | | AART | | VYYCNA | | | |
| 5hvh_B | QVQLQESGGG | SIFSGNA | MGWYRQA | SGGS | TDYADSVKGRFTISRDNA | DPRPWGYDVTDY | DYWGQG | Vicugna |
| | LVQPGGSLRLS | | PGKQRELV | | KNTVYLQMNSLKPEDTAV | | TQVTVSS | |
| | CAASG | | AAIT | | YYCHV | | | |
| 5j1t_C | QVQLVETGGG | NIFSFNV | MGWYRQA | SGDT | TTYADSVQGRFTISRDNA | RRNPINGPYYTT | AYWGQGT | Vicugna |
| | LVQAGGSLRLS | | PGKQRELV | | KNAVYLQMNSLTPEDTAV | | QVTVSS | |
| | CAASG | | AAIT | | YFCNA | | | |
| ATL24970.1 | QVQLVESGGG | FSGFTLDY | IGWFRQAP | SSSDG | TSYSDSVKGRFTISRDNA | RLGGWASFSPQE | DYWGQG | Vicugna |
| | LVQPGGSLRLS | YA | GKEREGLS | F | KNTVYLQMNSLKPEDTAV | Y | TQVTVSS | |
| | CVASE | | SIS | | YYCAA | | | |
| ATL24992.1 | QVQLAETGGG | FRLQYYT | AGWFRQAP | AGGGV | TYYTGSVQGRFTISRDNA | DLEYSQIMPSCR | GVRGQGT | Vicugna |
| | LVEPGGSLRLS | | GKEREWVA | | KRTVYLQMDSLKPEDTAV | GSY | QVTVSS | |
| | CAAPE | | CIS | | YSCAA | | | |
| 5lhr_B | QVQLQESGGG | RTFSSYV | MGWFRQAP | WSGGS | TNYADSVKGRFTISRDNA | DLASSRDVSS | WYWGQG | Vicugna |
| | LVQAGGSLRLS | | GKEREFVA | | KNTVYLQMNSLKPEDTAV | | TQVTVSS | |
| | CAASG | | AIS | | YYCAA | | | |
| 5nbm_F | QVQLVESGGG | RTFTNYG | MGWFRQAP | RFGFP | LYYSDSVKGRFTISRDNA | RNPRATAEDVSA | DYWGQG | Vicugna |
| | SVQAGDSLRLS | | GKEREFVA | | KTTVYLQMHNLKPEDTAI | Y | TQVTVSS | |
| | CTASG | | AIG | | YFCAA | | | |
| 5o02_C | QVQLQESGGG | FTFSSYA | MRWYRQAP | SAGGS | THYADSVKERFTISRDNA | RRDYGDSWFTAG | GYWGQG | Vicugna |
| | LVQPGGSLRLF | | GKERELVA | | KNTMYLQMNSLKPEDTA | G | TQVTVSS | |
| | CAASG | | AIT | | VYYCNA | | | |
| 5o04_F | QVQLQESGGG | IIFFMYD | VGWYRQAP | SDVS | TKYADSVKGRFTISRDNA | RRASA | DYWGQG | Vicugna |
| | LVQPGGSLRLS | | EKQRELVA | | KRTVYLQMNDLKPEDAA | | TQVTVSS | |
| | CTAPR | | QIN | | VYYCNV | | | |
| 5o05_D | QVQLQESGGG | SVSRTYV | MGWYRQTP | SVGS | TNYADSLKGRFTISRENA | IRYSPIHAPL | DYWGQG | Vicugna |
| | LVQPGGSLRLS | | GNQRELVA | | ENTVYLQMNSLKPEDTAI | | TQVTVSS | |
| | CAASG | | TIT | | YYCKY | | | |
| 5o0w_H | QVQLQESGGG | TALTYYA | IGWFRQAP | RINSGS | TDYADSVKGRFTISRDDA | DTTDRYDSANGR | DYWGQG | Vicugna |
| | LVQPGGSLRLS | | GKEREGVS | GAR | KNTVTLQMNSLEPEDTAR | YYCTISSDTY | TQVTVSS | |
| | CAASE | | CIS | | YYCAL | | | |
| 5ocl_H | QGQLVESGGG | IIFRELG | IDWYRQAP | SAGM | TNYADSVKGRFTISRDNA | LPRFS | HLWGQGT | Vicugna |
| | LVQAGGSLRLS | | GKQRELVA | | KNTVYLQMNSLKPEDTAV | | RVTVSS | |
| | CAASG | | SIA | | YYCHT | | | |
| 5omm_C | QVQLQESLVGL | NINSMHV | VGWYRQAP | DDGS | TDYVDSVKGRFTISRDIAE | TIVVFTTPM | HYWGKGT | Vicugna |
| | VQSGGSLRLSC | | GNQRELVA | | NTVYLQMNSLNPEDTAV | | QVTVSS | |
| | AASR | | SIT | | YYCKG | | | |
| 5omn_C | QVQLQESGGG | TIFSRNI | MGWYRQA | SDRS | TWYAESVEGRFTISRDNV | RTL | GSWGQG | Vicugna |
| | LVQPGGSLRLS | | PGKERELV | | KNTLYLQMNSLKPEDTA | | TQVTVSS | |
| | CAASG | | ASIY | | MYYCRD | | | |
| 5td8_E | QVQLVESGGG | RTGSRHA | VAWFRQAP | AVGLV | RNYADSVLGRFSISRDFA | RYYSGTYSSTYD | DYWGQG | Vicugna |
| | LVHPGGSLRLS | | GKERDFVA | | KNEVYLQMNSLEPEDTA | RDDY | TQVTVSS | |
| | CAASG | | SIN | | VYYCAA | | | |
| ANJ89050.1 | QLQLVESGGGL | RTSTSYA | QGWFRQPP | THDGN | THYTDSVKGRFTISRDNA | SLGYSGAYASGY | DYWGQG | Vicugna |
| | VQAGGSLRLTC | | GKEREFVA | | ENTLYLQMNSLKTEDTAV | | TQVTVSS | |
| | ALSE | | SLR | | YYCAA | | | |
| ANJ89062.1 | QLQLVESGGGL | RPFRNYR | LGWFRQAP | WGGVI | TNYADSVKGRFTISRDDA | FDSTGSPIVRDNY | EYWGQGT | Vicugna |
| | VQAGGSLTLSC | | GKEREFVA | | KNTVYLQMNSLKPEETAV | | QVTVSS | |
| | VASG | | AIN | | YYCAA | | | |
| 5vxj_J | QVQLAETGGG | FTFSRAV | MNWYRQAP | DAGGN | GSIADPVKGRFTISRDNA | GIFDGNYR | TYWGQGT | Vicugna |
| | LAQPGGSLRLS | | GKERELVA | | KNTVHLQMNSLKPEDTA | | QVTVSS | |
| | CAASG | | RIY | | MYVCNA | | | |
| 5vxl_B | QVQLAESGGG | GVFIIYN | MGWYRQA | SYSGSI | TNYADSVKGRFTISRDNV | NLRTN | NYWGQG | Vicugna |
| | LVQPGGSLRLS | | PGKQRELV | | EKRVYLEMNNLKPEDTAV | | TQVTVSS | |
| | CSASG | | ASID | | YYCNA | | | |
| 6cwk_A | QVQLAESGGG | RDFSMYM | LAWFRQAP | CSGGG | TYYADSMQGRFTISRDNA | STTYCSATTYSSD | DFWGQGT | Vicugna |
| | LVQAGGSLKLS | | GKEREFVA | GG | KKTVALQMNSLKPEDTAV | RLY | QVTVSS | |
| | CAASG | | AIM | | YYCAA | | | |
| 6h6y_H | QVQLQESGGG | RTFSNYY | SGWFRQAP | WSDST | TNYADSVKGRFTISRDTA | RRLATY | DYWGQG | Vicugna |
| | LVQAGGSLRLS | | GKEREFLAS | | KNTVYLQMNSLKLEDTAV | | TQVTVSS | |
| | CAVSG | | IR | | YHCAA | | | |
| 6h70_D | QVQLQESGGG | RTIDVSV | MAWFRQAP | WSGMT | TYSADSVKDRFTISRDKT | RSRFIVGVPQARD | DYWGQG | Vicugna |
| | LVMTGGSLRLS | | GKEREFVS | | KNTVYLQMNSLKPEDTAV | LY | TQVTVSS | |
| | CAVSG | | GMR | | YYCAA | | | |
| 6h72_D | QVQLQESGGG | RMFSINS | MGWYRQA | EAGT | TTYADSVRGRFTIARDNA | YIQLDSTIWFR | AYWGQGT | Vicugna |
| | LVQAGGSLRLS | | PGKERELV | | KNTVYLQMNSLNPEDTA | | QVTVSS | |
| | CAASG | | ATIS | | VYYCNA | | | |
| 6iyn_A | QLQLVESGGGL | RTFSSYA | MGWFRQAP | WSGRR | TYYADSVKGRFTISRDNA | GKWDGSYYGAP | DYWGQG | Vicugna |
| | VQAGGSLRLSC | | GKEREFVA | | KNTVYLQMNSLKPEDTAV | | TQVTVSS | |
| | AASG | | VVN | | YNCAA | | | |
| ABO38189.1 | QLQLVESGGGL | VTLEEVS | VAWFRQAP | SNGNG | TTYARSVKGRFTISRDSA | VEPNCSSGRKAF | GSWGQG | Vicugna |
| | VQPGGSLQLA | | GKEREGVS | GS | KNAVYLQMNSLKSEDTA | | TQVTVSS | |
| | CEASG | | CIN | | VYYCSV | | | |
| ABO38191.1 | EVQLVESGGG | SLASMNS | MGWYRQS | RSNY | TIYSTSVKDRFILSRDHDK | EGRGWG | PVLGQGT | Vicugna |
| | SVQAGGYLRLS | | PEKEREFVA | | NILSLQMNGLKPEDTAMY | | QVTVSS | |
| | CAVSG | | SIN | | YCAA | | | |
| ABO38192.1 | EVRLEESGGGL | TIFSYNA | MGWYRQP | AQG | TIEYAEAVKGRATISRDNA | WDRSDYRGHGH | DFLGQGT | Vicugna |
| | VQAGGSLRLVC | | PGKERELV | | KKLSLQLNNLKDEDTAMY | | QVTVSS | |
| | VAYG | | AGIT | | YCAA | | | |
| ABO38195.1 | QEQFVETGGG | GAVYHRS | LGWYRQAP | SGGG | TGYADFVKGRFTLSRPFG | WPMKA | STWGQGT | Vicugna |
| | LVQPGGSLTLS | G | GKERAWVG | | NNTVFLQMNNLEPEDTA | | QVTVSS | |
| | CSVYG | | RIF | | VYYCNT | | | |
| ABO38196.1 | QEQLVESGGG | FTIENYH | VRYYRQAP | VGNAW | TSYADSVKGRFTISRDSD | RVFSA | PCAGQGT | Vicugna |
| | LVQPGGSLRLS | | GKECELVA | | NKTVYLQMKSLQPEDTA | | QVTVSS | |
| | CAASG | | GIA | | VYHCNV | | | |
| ABO38199.1 | QVQLVESGGG | SNLRLYG | MGWFRQAP | WIGGA | AYYSTSVKGRFTLTRDYD | STTPPIFTF | NSYGPGT | Vicugna |
| | FVQAGGSLNLS | | GSERELVA | | QKTTLLQMNKLSPEDTAV | | QVTVSS | |
| | CTASG | | QIS | | YFCAA | | | |
| ABO38202.1 | QLHFVESGGG | FTLNYYS | IAWFRRAP | GGGV | TVYAESVKGRFTISRDSA | DEEAVCSVATLKR | DWWGQGI | Vicugna |
| | SVQPGGSLRLT | | GKEREGVS | | KNTMYLEMNSLEPEDTA | RAKY | QVTVSS | |
| | CAAST | | CMS | | EYYCAA | | | |
| ABO38206.1 | QVRPVESGGG | VNLNNFA | IGWFRQAP | NRGT | PYYADFLEGRFTISRDNA | DLGAGGCVLSPA | EVMGPGT | Vicugna |
| | SVQPGEALRLS | | GKEREGVS | | KNTVYLQMNDLKPEDTG | GTLSIARFL | LVNVSS | |
| | CTQPK | | CIG | | VYSCAA | | | |
| ABO38209.1 | QLQFVESGGD | LNFDVLA | MGWFRQAP | NRGGH | TEYVDSVKGRFTISRDNA | PTALRWQCNPAY | GSWGQG | Vicugna |
| | SVQPGGSLRLS | | GKEREAVS | | KNTVYLQMDNLQVEDTA | F | TEVSVSS | |
| | CVASG | | CIS | | VYYCAA | | | |
| ABO38213.1 | QVKLVESGGG | FVLEIFV | IGWFRQVP | IRDGS | AYYEDSVKGRFTVSRDN | ESFWVGPVQAMC | HIWGQGT | Vicugna |
| | FVDAGGSLTLS | | GKEREGISC | | AENTVYLQMNNLKPEDT | NNP | QVTVSA | |
| | CASSG | | IS | | GVYTCAA | | | |
| ABO38215.1 | QPQVAESGGG | TTSSLDYIP | VAWFRQVP | GVPHG | IFYSTPVKGRFTSSRDDA | ANKGFCTLDAND | KYWGQGT | Vicugna |
| | LVQPGGSLTLS | | GKEREGISC | EDH | KNTVYLHMNRLTPGDTA | Y | QVTVSS | |
| | CVVTG | | IG | | VYYCSP | | | |
| ABO38221.1 | QLHVVESGGG | FTLDSHH | IGWFRQAP | TTGDF | PHYADSVKGRFTISRDNA | DSGSRSSWSLCP | DYWGQG | Vicugna |
| | LVQPGGSLRLS | | GKERELVS | | KNTVYLQMNSLKPEDTAV | RGSVGY | TQVTVSS | |
| | CAASG | | CTG | | YYCAA | | | |
| ABO38222.1 | QLHVVESGGG | LDLDYLT | LGWFRQAP | HSGDL | EVYGDSVRGRFAISRDNA | DPARFTTCSPDE | DYWGQG | Vicugna |
| | LVQPGGSLRLS | | GKEREWVS | | KNTVYLQMNRLEPKDQA | Y | TQVTVSS | |
| | CKVSG | | CVD | | VYYCAA | | | |
| ABO38223.1 | QLQLVESGGGL | SSLDSYA | IGWFRQAP | SSGGT | TNYADSVKGRFTISRDNA | APGLTTFQTLCLM | DYWGQG | Vicugna |
| | VQPGGSLRLSC | | GKEREGVS | | KNTVYLQMNSLKPEDTAV | IGG | TQVTVSS | |
| | AASG | | CIN | | YYCAA | | | |
| ABO38224.1 | QLQLVESGGGL | SSLDSYA | IGWFRQAP | SSGGT | TNYADSVKGRFTISRDNA | GSGSTYYDCSGY | DKWDQGT | Vicugna |
| | VQPGGSLRLSC | | GKEREGVS | | KNTVYLQMDSLKLEDTGI | ETGWRI | QVTVSS | |
| | AASG | | CIN | | YVCAA | | | |
| ABO38226.1 | RLHLVESGGGS | EPLENNA | VGWFRQSP | TLGSR | DDYAGSVKGRFTVSRND | VYTGAGQLLPSLC | DYWGQG | Vicugna |
| | VQPGGSLRLSC | | GNSREGISC | GIT | AKNTVYLQMNNLKPEDT | LDENGY | TQVTVSS | |
| | VASG | | IS | | AVYFCAA | | | |
| ABO38227.1 | QVEPVESGGG | EALYHYA | VGWFRQVP | SSGEN | VDYHESVKGRFTISKDST | ASDLWYSGLYCG | DYWGQG | Vicugna |
| | LVQPGGSLRLS | | GRKREWVA | | RNTAYLDIMNIEPEDTATY | PDY | TRVAVSS | |
| | CTSHI | | CIS | | YCGA | | | |
| ABO38228.1 | QLQPARAGGN | RTLNGYD | IGWFRQAY | RSGN | TVYADSVKGRFTVARDN | LGGDVCVRFGRP | DYWDQGT | Vicugna |
| | LVQPGGSLRLF | | GTEREGISC | | AKTATFLEMSDLKPEDTG | GAYYCIANRCYD | QVTVSS | |
| | CGVSE | | TS | | TYFCAA | | | |
| ABO38229.1 | QLQLVESGGGL | SSLSYYH | IGWFRQAP | DSGGS | ITYADSVKGRFTISRDDAE | VTPGYYGGSYSCI | GVWGQG | Vicugna |
| | VQPGGSLRLSC | | GKGREAVA | | NTVHLQMTSLKAEDTAIY | PD | TQVTVSV | |
| | VASG | | CIS | | YCAP | | | |
| ACS73863.1 | HVQLQQSGGG | SIFSIYA | MGWYRQA | SYGS | TNYADSVKGRFTISRDNA | DIATMTAVGGF | DYWGQG | Vicugna |
| | LVQPGGSLRLS | | PGKQRELV | | KNTVYLQMNSLKPEDTAV | | TQVTVSS | |
| | CAASG | | AAIS | | YYCNA | | | |
| ACS73868.1 | QVQLVESGGG | SVFTMYT | TAWYRQTP | DEHR | TNYAASAEGRFTISRDNA | EHDLGYY | DYWGQG | Vicugna |
| | SVQPGGSLRLS | | GNLRELVAS | | KHTVDLQMTNLKPEDTAV | | TQVTVSS | |
| | CAAIG | | IT | | YYCKL | | | |
| ACZ53930.1 | QVQLVESGGG | FTLEYYA | IGWFRQAP | SSGGG | TNYADSVKGRFTISRDNA | DDFRCGSRWAAY | SSWGQGT | Vicugna |
| | LVQPGGSLRLS | | GKGREGVA | | KKMVYLQMNSLKSEDTA | LR | QVTVSS | |
| | CATSG | | CMN | | VYYCVV | | | |
| ACZ80603.1 | DVQLVESGGG | IIFSINA | MGWYRQA | SNGN | TLYADSVKGRFTISRDNA | PDDEY | DYWGQG | Vicugna |
| | LVQAGGSLRLS | | PGKERELV | | KNTLYLQMNSLKPEDTA | | TQVTVSS | |
| | CAASG | | AAIS | | MYYCTA | | | |
| AEJ91544.1 | QLQLVESGGGL | GNLDYYA | IGWFRQAP | SSDGS | TVYTDSVKGRFTISRDNT | VVNYYCTAGGSIH | EIWGQGT | Vicugna |
| | VQPGGSLRLSC | | GKEREGVS | | KNTVDLQMDNLKPEDTA | ASPY | QVTVSS | |
| | AASA | | CIS | | VYYCAT | | | |
| AEJ91545.1 | QVQLVESGGG | SLPSTPFN | VGWYRQAP | LR | INYADSVKGRFTISRDNA | EYT | HYWGKGT | Vicugna |
| | LVHPGGSLRLS | PFNNM | GKQREMVA | | KNTVDLQMDSLRPEDSA | | LVTVSS | |
| | CAPSA | | SIG | | TYYCHI | | | |
| AEJ91548.1 | QVQLVESGGG | FTFGDWV | MSWFRQAP | ATSSL | KYYADSVKGRFTISRDNV | P | NYWGQG | Vicugna |
| | LVQPGGSLRLS | | GKEREFVA | | NNTLFLQMDRLKSEDTAV | | TQVTVSA | |
| | CQVSG | | SIT | | YYCRS | | | |
| AEJ91549.1 | QVQLVESGGG | SDISGIA | MGWYRQA | SGGS | TDYAGSVKGRFTISRDNA | YGSG | DYWGQG | Vicugna |
| | LVQVGGSLRLS | | PGKRREMV | | KKTSYLQMNNVKPEDTG | | TQVTVSS | |
| | CVVSG | | ADIF | | VYYCRL | | | |
| AEJ91550.1 | QVQLVETGGG | FGMDYYY | IGWVRQAP | NIGR | THYADSVKGRFTISRDNA | APLVGNYCPASY | ESWGQGT | Vicugna |
| | LVQPGGALRLS | | GKEREGVS | | KNTVYLQMNSLKPEDTAV | EY | QVTVSS | |
| | CAASV | | CIS | | YYCAA | | | |
| AEJ91551.1 | QVQLVETGGG | RTSSFYA | LAWFRQGP | WIDGS | TRYTDSAKGRFTISRDAA | RTQYGGSSADPK | GYWGQG | Vicugna |
| | LVQAGGSLRLS | | GKEREFVA | | KNTMYLQMNSLKPEDTA | NY | TQVTVSA | |
| | CTASG | | AIG | | VYSCTA | | | |
| AEJ91553.1 | QVQLVETGGG | RSFSAAV | MGWFRQAP | QIIGS | THYADSVKGRFTISRDNA | GPPTMLDVSTDR | DTWGQGT | Vicugna |
| | LVQAGGSLRLS | | GKEREFVA | | KNMLYLDMNSLKPTDTAA | EY | QVTVSS | |
| | CAGSG | | ALR | | YYCTA | | | |
| AER93245.1 | QVQLQESGGG | LIFRFSA | MSWYRQG | SNGGT | TNYADFAKGRFTISRDNA | GLRMDS | RDWGQG | Vicugna |
| | LVQPGGSLRLS | | PGKERELV | | KNTVDLQMNSLKPEDTA | | TQVTVSS | |
| | CAASG | | AGIT | | VYYCHA | | | |
| AER93247.1 | QVQLQDSGGG | LSFRSYG | MGWFRQAP | WIGGF | THYTDSVKGRFTISRDNA | PGAPYSDRGRNA | GYWGQG | Vicugna |
| | LVQAGGSLRLS | | GKEREFVA | | KNTLYLQMNSLKPEDTAV | SEY | TQVTVSS | |
| | CAVSG | | RIS | | YYCAA | | | |
| AER93255.1 | QVQLQDSGGG | IILRFTS | MAWYRQAP | SSGK | TNYADSVQGRFTISRDNV | Q | DLWGQGT | Vicugna |
| | LVQPGGSLRLS | | GKQRELVA | | NKAVYLQMNSLDPEDTA | | QVTVSS | |
| | CATSG | | RIT | | VYYCNT | | | |
| AER93251.1 | QVQLQDSGGG | RTFRLYN | MGYFRQAP | WLGGR | AYYADSVKGRFTISRDGA | KNRGSDSDVVRG | DYWGQG | Vicugna |
| | LVQAGGSLRLS | | GKEREFVA | | KNTLSLQMSSLKPEDTAV | Y | TQVTVSS | |
| | CTASG | | AIT | | YYCAA | | | |
| AER93256.1 | QVQLQESGGG | FTFSRYT | MSWYRQAP | KSGTA | INYADSVKGRFTISRDNA | GGFTIAPLD | SYWGQGT | Vicugna |
| | LVQPGGSLRLS | | GKERELVA | | KNTLYLQMNSLKPEDTAV | | QVTVSS | |
| | CAASG | | AIT | | YYCAR | | | |
| AGY32871.1 | QVQLVETGGG | RTLDMYA | MGWIRQAP | GVGGS | PRYADSVKGRFTISKDNT | GGDIYYGGSP | QWRGQG | Vicugna |
| | LAQAGDSLRLS | | GEEREFVA | | KSTIWLQMNSLKPEDTAV | | TRVTVSS | |
| | CVEPG | | SIS | | YYCAA | | | |
| AGY32872.1 | QVQLVESGGG | RINGDYA | MGWFRQAP | WIGGS | TYYTDSVKGRFTLSRDNA | GHYTDFPTYFKEY | DYWGQG | Vicugna |
| | LVQAGGSLRLS | | GEEREFVA | | KNTLSLQMNSLKPEDTAV | | TQVTVSS | |
| | CAASG | | VNS | | YYCAA | | | |
| AGY32875.1 | QVQLVESGGG | FTLADYV | TVWFRQAP | SSRGT | PNYADSVKGRATVSRNN | IRPARLRAYRECL | DYWGQG | Vicugna |
| | LVQPGGSLKLS | | GKSREGVS | | ANNTVYLQMNGLKPDDT | SSQAEY | TQVTVSS | |
| | CAASG | | CIS | | AIYYCAA | | | |
| AGY32877.1 | QVQLVETGGLV | VPFSDYT | MAWFRQAP | WRGG | PYYGNSGNGRFAISRDIA | SRLRPALASMAS | DYWGQG | Vicugna |
| | QAGGSLRLSCA | | GKEREVVA | G | KSMVYLHMDSLKPEDTA | DY | TQVSVSS | |
| | ASG | | RIT | | VYYCAA | | | |
| AHA34187.1 | QVQLVESGGG | FTLDDYA | IGWFRQVP | D | GSTYYADSVKGRFTISRD | RPCFLGVPLIDF | GSWGQG | Vicugna |
| | LVQPGGSLRLS | | GKEREGVA | | NGAVYLQMNSLKPEDTA | | TQVTVSS | |
| | CAASG | | CVK | | VYYCAS | | | |
| ATL25006.1 | QVQLVESGGG | GTFSDYG | MGWFRQAP | WNGN | IEYADSVKGRFTISRDNA | SISGYAYNTIERY | NYWGQG | Vicugna |
| | LVQAGGSLRLS | | GKEREFVA | GGNG | KNTVHLQMNSLTPEDTAV | | TQVTVSS | |
| | CAASG | | AIR | | YYCAA | | | |
| AHA34194.1 | QVQLVESGGLV | RADIIYA | MGWFRQAP | WSGGS | TYYADSVKGRFTISRDNA | RRSWYRDALSPS | DYWGQG | Vicugna |
| | QAGGSLRLSCA | | GKEREFVA | | KNSVYLQMNSLKPEDTA | RVYEY | TQVTVSS | |
| | ASG | | AVD | | VYYCAA | | | |
| AHA34196.1 | QVQLVESGGG | SIVSIYA | VGWYRQAP | TDGS | TKYSDSVKGRFTISRDNA | DAAGWGDQ | YYWGQGT | Vicugna |
| | LVQPGGSLKLS | | GKQRELLA | | KNTVYLQMNNLKPEDTAI | | QVTVSS | |
| | CRASG | | AIT | | YSCIG | | | |
| AHA34198.1 | QVQLVETGGAL | STFSSYG | MAWYRQAP | PDGT | PSYVNSVKGRFTISRDNA | WPRTMPD | ANWGRGT | Vicugna |
| | VHTGGSLRLSC | | GEQRKWVA | | KNSVYLHMNNLRPEDTA | | QVTVSS | |
| | EVSG | | GIM | | VYYCNQ | | | |
| AHA34199.1 | QVQLVETGGSL | STFNNPA | ITWYRQPP | SGDG | PVYRESVKGRFTIFRDNA | ASPASW | LDWGQGT | Vicugna |
| | RLTCVTSG | | GKQREWVA | | TDALYLRMNSLKPEDTAV | | QVTVSS | |
| | | | SLR | | YHCNT | | | |
| AHA34201.1 | QVQLVESGGG | SAVSDSFS | ISWHRQAP | NRGA | TSYRDSVKGRFTISRDNA | WPREG | LGGGQGT | Vicugna |
| | LVQAGGSLRLS | TYA | GKQREWIA | | KNTVYLQMNNLKPEDTG | | QVTVSS | |
| | CAASG | | GIS | | VYYCEP | | | |
| AHA34205.1 | QVQLVETGGG | PLFNLYD | MAWYRQAP | TDGR | ATYSDSVKGRFTISRNNL | KNSIYW | DSWGQG | Vicugna |
| | LVQAGGSLRLS | | GNQRELVA | | TNTVFLQMSSLKPEDTAV | | TQVTVSS | |
| | CVVSS | | GIL | | YYCNR | | | |
| AIO05488.1 | QLQLVESGGGL | RTFSRYA | MGWFRQAP | RSGVN | TRYEDSVKGRSTISRDNA | RRGPGTSVLSDD | DYWGQG | Vicugna |
| | VQAGGSLRLSC | | GKEREFVA | | KRSVFLQMDSLKRDDTA | Y | TQVTVSS | |
| | AASG | | AIS | | VYYCAA | | | |
| AIO05489.1 | QVQLVESGGG | RTFSRYT | MGWFRQAP | ARGT | IYYADSVKGRFTISRDNA | TRTPRVRLPTESR | TYWGQGT | Vicugna |
| | LVQAGGSLRLS | | GKEREFVA | | KNTVYLQMNSVEPEDTA | EY | QVTVSS | |
| | CAASG | | TIG | | AYYCAT | | | |
| ALD14642.1 | QVQLVESGGG | SSFSRYA | MRWYRQAP | SRGT | SNYADSVKGRFTISRDNA | EWLGRSE | PSWGQGT | Vicugna |
| | LVQPGGSLRLS | | GKQRELVA | | KNTVYLQMNSLKPEDTAV | | QVTVSS | |
| | CAASG | | NIN | | YYCNA | | | |
| ALD14643.1 | QVQLVESGGG | FIFSLYT | MRWHRQA | SATGI | TNYADSVKGRFIISRDDA | VRTTVSR | DYWGQG | Vicugna |
| | LVQPGGSLRLS | | PGKERELV | | KKTGYLQMNSLKPEDTA | | TQVTVSS | |
| | CAASG | | ATIT | | VYYCNA | | | |
| ALD14644.1 | QVQLVESGGG | IIFSIYT | MGWYRQA | SGPS | ANATDSVGGRFTITRDNA | RRGPGIK | NYWGQG | Vicugna |
| | LVQPGGSLRLS | | PGKQRELV | | ENTVYLQMNDLKPEDTA | | TQVTVSS | |
| | CAASG | | AAIP | | VYYCNA | | | |
| ALD14645.1 | QVQLVESGGG | SIARPGA | MAWYRQAP | PGGL | TNYADSVTGRFTISRDNA | RIIPLGLGSEYR | DHWGQG | Vicugna |
| | LVQPGGSLSVS | | GKERELVA | | KRTVYLQMNSLQPEDTA | | TQVTVSS | |
| | CAASG | | SIT | | VYYCHA | | | |
| ALD14646.1 | QVQLVETGGLV | LTFSSTA | MAWFRQAP | GAGIT | IYYSDSVKDRFTISRNNVE | RRNTYTSDYNIPA | PYWGQGT | Vicugna |
| | QPGGSLRLSCA | | GKEREFVA | | NTVYLQMNSLKTEDTAVY | RY | QVTVSS | |
| | ASG | | RIS | | YCAA | | | |
| ALD14647.1 | QVQLVETGGLV | STTATIYS | MNWYRQAP | SDGQ | TNYATSVKGRFTISRDNA | KPWRLQGW | DYWGQG | Vicugna |
| | QPGGSLRLSCA | | GKQRELVA | | KNTVYLLMNSLKLEDTAV | | TQVTVSS | |
| | ASR | | GMT | | YYCYV | | | |
| ALD14648.1 | QVQLVESGGG | SIVNSRT | MAWYRQAP | TAGS | PNYADSVKGRFAISRDNA | LLST | LPYGQGT | Vicugna |
| | LVQPGGSLRLS | | GKQRERVA | | KNTVYLQMNSLKPEDTAV | | QVTVSS | |
| | CAAPE | | TIT | | YYCNT | | | |
| ALD14649.1 | QVQLVESGGG | ERSINNYG | MGWYRQA | SGGT | TNYADSVEGRFTISRDNV | LLRT | FSWGQGT | Vicugna |
| | LVQPGGSLGLS | | PGKQRELV | | KKMVHLQVNSLKPEDTA | | QVTVSS | |
| | CVVAS | | AQIS | | VYYCNS | | | |
| ALD14650.1 | QVQLVETGGG | FTFSSYR | MSWYRQAA | ANGVP | TGYADSVMGRFTISRDNA | PRLHTSV | GYWGQG | Vicugna |
| | LVQPGGSLRLS | | GKERDVVA | | KNTVYLEMNSLNPEDTAV | | TQVTVSS | |
| | CAASG | | | | YYCNA | | | |
| ALD14651.1 | QVQLVESGGG | VIFSIYT | MGWFRQAP | VADGT | ALVADSVTGRFTISRDNA | YLSPRVQSPYITD | QLWGQGT | Vicugna |
| | LVQAGNSLRLS | | GKEREFVA | | KNTVYLHMNSLKPEDTAV | SRY | QVTVSS | |
| | CTASG | | AIG | | YSCAA | | | |
| ALD14652.1 | QVQLVETGGG | RYA | MGWFRQAP | RSGAI | REYADSVKGRFTISRDGA | GRGATFNPEY | AYWGQGT | Vicugna |
| | LVQAGGSLRLS | | GKEREFVA | | ENTVYLEMNSLKPDDTAI | | QVTVSS | |
| | CAASG | | TIS | | YVCAE | | | |
| ALD14653.1 | QVQLAETGGG | LTFGNYA | MGWFRQAP | RSGSN | TWYAEPLKGRFAISRDND | GSYNSDWWNY | MYWGQG | Vicugna |
| | LVQAGGSLRLS | | GKEREFVA | | KNALYLQMNSLKPEDTAV | | TQVTVSS | |
| | CSASG | | SIS | | YYCAG | | | |
| ALD14654.1 | QVQLVESGGG | GRTFSGYA | MGWFRQAP | WSGHN | TYYGDSVKGRFTISRDTA | EGARTHLSDSYY | GYWGQG | Vicugna |
| | GLVQAGGSLRL | | GKEREFVA | | KNTVYLQMNSLKPEDTAV | FPGLWAEPPV | TQVTVSS | |
| | SCAAS | | DIS | | YYCAA | | | |
| ALD14655.1 | QVQLVETGGG | RTFTSYY | IGWFRQEP | WTDDN | TYYADSVKGRFTISRDNA | DYGSGIRAWYNW | IYWGQGT | Vicugna |
| | LVQAGGTLRLS | | GKEREFVA | | ETTAYLQMSGLKPEDTAV | | QVTVSS | |
| | CAASG | | SIG | | YYCAA | | | |
| ALD14658.1 | QLQLAESGGGL | IIFDYYS | VDWYRQAP | GDGS | PNYADSVKGRFTISRDNA | KRTIGTKS | EYWGQGT | Vicugna |
| | VQPGGSLRLSC | | GKERELVAT | | KKTVYLQMNGLKPEETAV | | QVTVSS | |
| | TASG | | IT | | YYCHA | | | |
| ALD14659.1 | QVQLAETGGG | MSFSRRP | MAWYRQAP | SFGDT | TNYTDSVEGRFTISRDNA | LLAT | YAWGQGT | Vicugna |
| | LVQAGGSLRLS | | GKQRERVA | | KNTMYLQMNSLKPDDTA | | QVTVSS | |
| | CLASR | | TIS | | VYYCNT | | | |
| ALD14660.1 | QVQLAESGGG | RTFSSYV | MGWFRQAP | RNGGK | TYYADSVKGRFTISRDGT | AVAASAEFVTARS | EYWGQGT | Vicugna |
| | LVQAGGSLRLS | | GKEREFVA | | ENTVYLQMNSLKPEDTAV | NFY | QVTVSS | |
| | CAASG | | AIS | | YYCAA | | | |
| ANJ89048.1 | QLQLVESGGGL | LSLSDYA | MGWFRQAP | WNGLR | IAYADFVKGRFTISRDNAK | ARSEWGSRAVY | DYWGQG | Vicugna |
| | VQAGGSLRLSC | | GKEREFVS | | NTAYLQMNSLKPEDSAV | | TQVTVSS | |
| | AASG | | GIG | | YYCAA | | | |
| ANJ89051.1 | QVQLVETGGG | GTLRNYD | MAWFRQAP | RNGHI | ISYSDSARGRFTISKDSAK | GSGVGYAFQPTR | QDWGQG | Vicugna |
| | LVQPGGSLRLS | | GKEREFVA | | NALYLQMNDLKPEDTAVY | | TQVTVSS | |
| | CAASG | | SIG | | YCAA | | | |
| ANJ89052.1 | QLQLVESGGGL | GTVSTID | MGWFRQVP | SSGHV | TSTGDSVKGRFTISKDNA | GNWNSRAREY | DSWGQG | Vicugna |
| | VQRGGSLRLS | | GKEREFVA | | KNTVYLQMNDLKPEDTA | | TQVTVSS | |
| | CAASG | | GMS | | VYYCAS | | | |
| ANJ89059.1 | QVQLVESGGG | RTLSTKT | MGWFRQTP | WIGD | TYYANSVKGRFTISRDIAK | ADPVRRPYYYEN | NYWGQG | Vicugna |
| | LVQAGGSLRLS | | GKERDFVA | | NTVYLQMHSLTPEDTAVY | NNNY | TQVTVSS | |
| | CATAG | | AIT | | YCAA | | | |
| ANJ89060.1 | QLQLVESGGDL | GTLSSYA | MGWFRQAP | WSGKR | IAYADFVKGRFTISRDNAK | ARSEWGSRAEY | DYWGQG | Vicugna |
| | VQAGGSLRLSC | | GKEREFVA | | NTVYLQMNSLKPEDSAV | | TQVTVSS | |
| | AASG | | GIG | | YSCAA | | | |
| ANJ89061.1 | QVQLVETGGG | IIFRPNY | MGWYRQA | SGGT | THIADSVKGRFTISRDND | RRSMASNPY | DYWGQG | Vicugna |
| | LVQAGGSLRLS | | PGKQREPV | | KNTVDLQMNSLKPEDTA | | TQVTVSS | |
| | CAASG | | ATLT | | VYYCNY | | | |
| ANJ89063.1 | QVQLVETGGG | SIASLNG | MGWYRQG | NIGE | TYYADSVKGRFTISRDNA | PLRTM | EYWGKGT | Vicugna |
| | LVQAGGSLRLS | | PGKQRELV | | NKTVSLQMNSLKPEDTAV | | LVTVSS | |
| | CVGSG | | ARFT | | YYCNY | | | |
| ATL25005.1 | QVQLAESGGG | RTFNKNA | MAWFRQTP | WTGNS | PYYADSVKGRFAISRDDA | SIYFASATGDQSR | DYWGQG | Vicugna |
| | LVQAGGSLRLS | | GKEREFVA | | KDALYLQMNSLKPEDTAV | VY | TQVTVSS | |
| | CAASG | | SID | | YYCAV | | | |
| ATL24953.1 | QVQLAETGGG | SIFSIWA | MGWYRQG | TDGGR | TNTADSLKGRFTISRDNA | GHRSTWAGYPAA | DYWGQG | Vicugna |
| | LVQPGGSLRLS | | PGTLRELVA | | KNTVYLQMNNLKPEDTA | D | TQVTVSS | |
| | CAASG | | SIT | | VYYCFA | | | |
| ATL24954.1 | QVQLAETGGG | TTFNMYA | MGWYRQA | NTGI | INYADSVKGRFTVSRDNG | RAPTGEGGW | TLMGQGT | Vicugna |
| | LVQAGGSLRLS | | QGNQRELV | | KNTVSLQMNSLKPEDTAV | | QVTVSS | |
| | CGASG | | AIAS | | YFCYA | | | |
| ATL24957.1 | QVQLVETGGG | GTFSSNA | MGWFRQTP | WSASS | TYYRDSVKGRFTVSRDN | SSVYAEMPYAAS | DFWGQGT | Vicugna |
| | TVQIGGSLRLL | | GKEREFVA | | AKNTVYLQMNSLKLEDTA | VKTTSY | QVTVSS | |
| | CSGHG | | GIN | | IYYCAG | | | |
| ATL24958.1 | QVQLAESGGS | STFDDYA | VGWYRQVP | NTGS | PAFADSVKGRFTISRDNA | WPFTKL | PSWGPGT | Vicugna |
| | VQAGGSLRLSC | | GGHRAWVA | | KKTVYLTMNSLKPEDTAI | | QVTVYS | |
| | VVSG | | GIS | | YYCNL | | | |
| ATL24961.1 | QVQLVESGGG | FPFSSYV | MSWYRQAP | SFGTN | TMYANSAKGRFTISRSDG | RNELRD | IYWGQGT | Vicugna |
| | LVQPGGSLRLS | | GKEREWVA | | HNTLYLQMNSLKPEDSAV | | QVTVSA | |
| | CAASG | | TSF | | YFCHA | | | |
| ATL24962.1 | QVQLAESGGG | VTVRDVP | MGWYRQA | NGGLR | TDYTDSVKGRFTISRDDA | LIQWR | DYWGQG | Vicugna |
| | LVQAGGSLRLS | | PGTQREWV | DY | KNTWYLQMNSLKPEDTA | | TQVTVSS | |
| | CAASG | | ARIS | | VYFCKT | | | |
| ATL24964.1 | QLQLVETGGDL | TTLFIAA | MGWYSQAP | SGGR | TNYRDSVKGRFTVSRDL | ERLGK | EYWGQGT | Vicugna |
| | VQAGGSLRLSC | | GKQRELVA | | ANNTVHLEMTSLKPEDTA | | QVTVSS | |
| | AAPR | | TLT | | VYYCYA | | | |
| ATL24965.1 | QVQLAQTGGG | ITDSNYA | ITWYRQPP | RDG | AVYRNSVQGRFTISGHSA | PSPASW | LPWGQGT | Vicugna |
| | LVQTGESLKLS | | GKQREWVA | | ENAVYLQMDSLKPEDTA | | QVTVSS | |
| | CTTSG | | SIT | | VYYCNT | | | |
| ATL24967.1 | QLQLVETGGGL | SFFSMHD | VGWYRQAL | NDGL | TTYADSVKGRFTISRDNT | RFGNDTNR | NYWGQG | Vicugna |
| | VQTGGSLRLSC | | GKQRELVA | | KKTGWLEMNMVKPEDAA | | TQVTVSS | |
| | AASG | | LLN | | VYYCNA | | | |
| ATL24969.1 | QLQLVETGGGL | FTLGHYA | TGWYRQAP | SSGGS | TYYADSVKGRFAISRDNA | EAYGSGCPRVR | DYWGQG | Vicugna |
| | VQPGGSMRLS | | EKEREWVS | | KNTVYLQMNSLKPEDTAV | | TQVTVSS | |
| | CAASG | | CIS | | YVCEV | | | |
| ATL24972.1 | QVQLVESGGG | RTFSTPGR | LGWFRQAP | WGVDD | ADYAASVKGRFTISGVNA | GLRGGTYARIQY | DYWGQG | Vicugna |
| | LVQAGGSLRLS | TFSDYG | GKEREFVA | AYY | KNTVYLQMNSLKPEDTAV | DY | TQVTVSL | |
| | CEASG | | SIS | | YYCAA | | | |
| ATL24973.1 | QVQLAETGGG | SISSFYF | MGWYRQA | NYGR | TDYGDSLKGRFTISRDNA | RKWERSVLE | DYWGQG | Vicugna |
| | LVQPGGARTLS | | PGKPRELV | | ANTVNLQMNNLAPEDTAL | | TQVTVSS | |
| | CAASE | | AEIS | | YYCNA | | | |
| ATL24976.1 | QVQLVESGGG | FTFSSTP | MNWFRQAP | SRNDI | AYYADSVKGRFTVSRDD | PAGRIED | ELWGQGT | Vicugna |
| | LVQPGGSLRLS | | GKEREFVA | | AKNTVYLQMNSLKPEDT | | QVTVSS | |
| | CVASG | | GVG | | GVYYCKR | | | |
| ATL25009.1 | QVQLAESGGG | FTFSTSP | MSWFRQAP | SRNDI | TYYADFVKGRFTISRDNA | PARQIEY | ENWGQG | Vicugna |
| | LVQPGGSLRLS | | GKEREFVA | | KNVAYLQMNNLKPEDTG | | TQVTVSS | |
| | CAASG | | NVG | | VYYCKR | | | |
| ATL24979.1 | QVQLVETGGG | RTFSVSGR | LGWFRQAP | WSVDG | TDLANSVKGRFTISGVNA | GLRGGTYARTIYE | DYWGQG | Vicugna |
| | VVQAGGSLRLS | TFSDHG | GKEREFVG | DATYY | KNTVYLQMNSLKPEDTAV | Y | TQVTVSL | |
| | CVASG | | SIS | | YYCAA | | | |
| ATL25012.1 | QVQLVESGGG | VTFSDYY | IGWHRQDP | WRGDV | TYYGDSVKGRFTISRDNA | YREGI | EYWGKGT | Vicugna |
| | LVQPGGSLRLS | | GKEREFVG | | RNTVYLQMNSLKAGDTA | | QVTVSS | |
| | CAASG | | AVN | | VYYCKA | | | |
| ATL25007.1 | QLQLAETGGGF | RTFSNAL | MAWFRQAP | WSTNN | TRHADSVKGRFTISGDNA | DTSLDRWLRNGN | RYWGRGT | Vicugna |
| | VQAGGSLRLSC | | GKEREFVA | | KNTVYLQMNSLKPEDTAV | P | QVTVSS | |
| | VASG | | RIN | | YYCAG | | | |
| ATL24983.1 | QLQLVESGGSL | FTFSAKG | MSWYRQAP | HTGEN | VNYADSVKGRFRISRNNA | RRILRP | DFWGQGI | Vicugna |
| | KLSCAASG | | GKERELVA | | KNMLYLHMTSLKPEDTAV | | EVTVSP | |
| | | | YIS | | YYCAA | | | |
| ATL24985.1 | QLQLAETGGGL | RSFSLAS | MGWFRQAP | RSISL | TKYADFVKGRYTISRDNV | DLGAWSSVIVEPT | DYWGQG | Vicugna |
| | VQAGGSLRLSC | | GKEREFVA | | KNTVYLQMNTLKPEDTAV | YEW | TQVTVSP | |
| | AASG | | AVS | | YYCAA | | | |
| ATL24986.1 | QVQLVESGGG | SISSIDV | MGWYRQA | SGGS | TDYAGSVKGRFTISRDNA | EKLRAGIPY | NYWGQG | Vicugna |
| | LVQAGGSLRLS | | SGKEREQV | | KNTVSLQMNSLKPEDTAV | | TQVTASS | |
| | CAASE | | AVST | | YYCQA | | | |
| ATL24990.1 | QLQLAETGGGL | SMFSMYN | ITWYRQPP | NIGG | TNYVDSVKGRFTISRDNA | KYSLRSSW | DYWGQG | Vicugna |
| | VQPGGSLRLSC | | GKQRELVA | | KKTAYLQMNSLKPEDTAV | | TQVTVSS | |
| | AASG | | AIT | | YYCNA | | | |
| ATL24993.1 | QVQLVETGGG | GTFSSYA | MGWFRQAP | LSGAG | TYYQDSVKGRFTISRDNA | TGERGYGDQGYL | EVWGRGT | Vicugna |
| | LVQAGGSLRLS | | GKERDFVA | | KNTVYLQMNSLKPEDTAV | | LVTVSS | |
| | CVASG | | GIS | | YYCKA | | | |
| ATL24995.1 | QVQLAETGGG | FSFSDYQ | MSWYRQAP | TTGTF | TQYADSVKGRFTISRDNA | VDLGGSF | GSWGQG | Vicugna |
| | LVQPGGSLRLS | | GKEREWVA | | KNTVYLQLNSVKPDDTAV | | TQVTVSS | |
| | CAASG | | LIT | | YYCKA | | | |
| ATL24996.1 | QVQLAETGGG | SMFSMYR | MSWYRQAP | SGGR | LNYAESVKGRFTISRDNG | YYSDNF | NGWGQG | Vicugna |
| | SVQPGGSLGLS | | GKERELVA | | KKTVYLQMNSLKPEDTAV | | TQVTVSS | |
| | CAASG | | SIG | | YYCNA | | | |
| ATL24997.1 | QVQLAETGGG | RTSSVYS | MGWFRQAA | STNEI | TYYADSVKGRFTVSRDN | PARTIEL | ENWGQG | Vicugna |
| | LVQAGDSLRLS | | GKEREFVA | | AKETVYLQMNNLKPEDT | | TQVTVSS | |
| | CTASG | | SVG | | GVYYCKR | | | |
| ATL24998.1 | QLQLAETGGGL | RMVSSRA | MGWFRQPP | WTGDS | TYYADSVKGRFTISRDNA | PAHFSGNYLSPH | EHWGQG | Vicugna |
| | VQAGGSLRLSC | | GKEREFVA | | NNTVYLQMNSLKPEDTG | VYEY | TQVTVSS | |
| | AASG | | AIS | | VYYCAA | | | |
| ATL25000.1 | QVQLVETGGG | FIFDNYA | IGWFRQAP | SHDGN | TYYADSVKGRFTVSRDS | DRYLYSGSGIVTY | EYWGQGT | Vicugna |
| | LVQAGGSLRLS | | GKEREGVS | | GKNTVYLEMNSLSPEDTA | CPNP | QVTVSS | |
| | CAASG | | CTS | | VYYCAA | | | |
| ATL25001.1 | QVQLAESGGG | GTFSDYG | MGWFRQAP | WNGN | IEYADSVKGRFTISRDNA | SISGYAYNAIERY | NYWGQG | Vicugna |
| | LVQAGGSLRLS | | GKEREFVA | GGNG | KNTVHLQMNSLTPEDTAV | | TQVTVSS | |
| | CATSG | | AIR | | YYCAA | | | |
| ATL25011.1 | QVQLAESGGG | RINSIYA | MDWHRQA | SGGT | ETYADAVKGRFTISRDNA | DVADPGYDD | HFWGQGT | Vicugna |
| | LAQSGGSLRLS | | PGKQREWV | | KNTAYLQMNHLKPEDTG | | QVTVSS | |
| | CVASG | | ARLT | | VYLCDV | | | |
| CAP58558.1 | EVQLQASGGG | FIFGWST | MSWYRQAP | GGGSA | TTYTDSVKGRFTISRDRA | DVSTGFRYQRK | DYWGRGT | Vicugna |
| | SVQPGGSLRLS | | GKQREWVS | | KNTLYLQMNSLKPEDTAI | | QVTVSS | |
| | CAASG | | IS | | YYCNA | | | |
| CAQ53176.1 | QVQLMESGGG | FTLDYYA | IGWFRQAP | SSDGS | TYYADSVKGRFTISRDNA | DRPPCYYCSGYG | GSWGQG | Vicugna |
| | LVQPGGSLRLS | | GKEREGVS | | KNTVYLQMNSLKPEDTAV | CYR | TQVTVSS | |
| | CAASG | | CIS | | YYCAA | | | |
| CAQ53177.1 | QLQLVESGGGL | SIFSINA | MGWYRQA | SGGS | TNYADSVKGRFTISRDNA | ESSWAEGGYL | EVWGQGT | Vicugna |
| | VQPGGSLRLSC | | PGKQRELV | | KNTVYLQMNSLKPEDTAV | | LVTVSS | |
| | AASG | | AAIT | | YYCNA | | | |
| CAQ53178.1 | QLQLVESGGGL | CTFSSYA | MGWFRQAP | WSGGS | TYYADSVKGRFTISRDNA | EAVVAGLLRPPPY | DYWGQG | Vicugna |
| | VRAGGSLRLSC | | GKEREFVA | | KNTVYLQMNSLKPEDTAV | EY | TQVTVSS | |
| | AASG | | AIS | | YYCAA | | | |
| CAQ53179.1 | QVQLVESGGG | RTFSSYA | MGWFRQAP | WSGGS | TYYADSVKGRFTISRDNA | DRSYTVVAGPRY | DYWGQG | Vicugna |
| | LVQAGGSLRLS | | GKEREFVA | | KNTVYLQMNSLKPEDTAV | EY | TQVTVSS | |
| | CAASG | | AIS | | YYCAA | | | |
| CAQ53181.1 | QVQLMESGGG | RTFSSYD | MGWYRQA | WSGGS | TYYADSVKGRFTISRDNA | DKDSDYDVQYGM | DYWGKGT | Vicugna |
| | LVQAGGSLRLS | | PGKEREFV | | KNTVYLQMNSLKPEDTAI | | LVTVSS | |
| | CAASG | | AAIS | | YYCNA | | | |
| CAQ53183.1 | QVQLMESGGG | RTFSSYA | MGWFRQAP | WSGGS | TYYADSVKGRFTISRDNA | APFPDGVLALGTI | GSWGQG | Vicugna |
| | LVQAGGSLRLS | | GKEREFVA | | KNTVYLQMNSLKPEDTAV | ATMPGFF | TQVTVSS | |
| | CAASG | | AIS | | YYCAA | | | |
| CAQ53185.1 | QLQLVESGGGL | FTLDYYA | IGWFRQAP | SSDGS | TYYADSVKGRFTISRDNA | DPNTDYGLEDLN | DYWGQG | Vicugna |
| | VQPGGSLRLSC | | GKEREGVS | | ENTVYLQMNSLKPEDTAV | PQEY | TQVTVSS | |
| | AASG | | CIS | | YYCAT | | | |

TABLE 11
Amino acid profile of natural VHHs and synthetic VHHs in the VHH input library. Position-wise amino acid profile
of natural VHHs and VHHs in the input library. Positions are relative positions within each segment and numbers
are percentage of the corresponding amino acid labelled to the left of each segment.
natural VHHs (298 unique VHHs from RSCB)

| | Frame1 | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 13 |
| P | 0 | 0 | 0 | 0.34 | 0 | 0 | 0 | 0 | 0 | 0 | 0.34 | 0 | 0.67 |
| A | 1.34 | 0 | 0.34 | 0 | 2.35 | 1.34 | 0 | 0 | 0 | 1.34 | 0.34 | 2.35 | 0 |
| G | 0 | 1.68 | 0 | 0.34 | 0 | 0 | 0 | 99 | 99 | 97.7 | 0.34 | 0 | 0 |
| M | 0 | 0.34 | 0 | 0.34 | 0 | 0 | 0 | 0 | 0 | 0 | 0.67 | 0.67 | 0.34 |
| I | 0 | 0 | 0 | 0 | 0 | 0 | 0 | 0 | 0 | 0 | 0 | 0 | 0 |
| L | 0 | 1.68 | 0 | 98.7 | 0 | 0 | 0 | 0 | 0 | 0 | 80.2 | 0.67 | 0 |
| V | 0 | 95 | 0 | 0 | 43.3 | 0 | 0 | 0 | 0.34 | 0 | 0.67 | 95.6 | 0.34 |
| F | 0 | 0 | 0 | 0.34 | 0 | 0 | 0.34 | 0 | 0 | 0 | 0 | 0 | 0 |
| W | 0 | 0 | 0 | 0 | 0 | 0 | 0 | 0 | 0 | 0 | 0 | 0 | 0 |
| C | 0 | 0 | 0 | 0 | 0 | 0 | 0 | 0 | 0 | 0.34 | 0 | 0 | 0 |
| S | 0 | 0 | 0 | 0 | 0.34 | 0 | 94 | 0 | 0 | 0 | 16.1 | 0 | 0 |
| T | 0 | 0 | 0 | 0 | 0 | 0 | 5.7 | 0 | 0 | 0 | 1.34 | 0 | 0.34 |
| N | 0 | 0 | 0 | 0 | 0 | 0 | 0 | 0 | 0 | 0 | 0 | 0 | 0 |
| Q | 77.2 | 0 | 96 | 0 | 51 | 2.01 | 0 | 0 | 0 | 0.34 | 0 | 0 | 95 |
| Y | 0 | 0 | 0 | 0 | 0 | 0 | 0 | 0 | 0 | 0 | 0 | 0 | 0 |
| H | 0 | 0 | 0 | 0 | 0 | 0 | 0 | 0 | 0 | 0 | 0 | 0 | 1.34 |
| R | 0 | 0.34 | 0.34 | 0 | 0 | 0 | 0 | 0 | 0 | 0.34 | 0 | 0 | 0.67 |
| K | 1.34 | 0 | 3.36 | 0 | 0 | 0 | 0 | 0 | 0 | 0 | 0 | 0 | 0 |
| D | 8.05 | 0 | 0 | 0 | 0 | 0.34 | 0 | 0 | 0 | 0 | 0 | 0 | 0 |
| E | 7.38 | 0 | 0 | 0 | 3.02 | 96.3 | 0 | 0.34 | 0 | 0 | 0 | 0.67 | 1.34 |
| | 4.7 | 1.01 | 0 | 0 | 0 | 0 | 0 | 0 | 0 | 0 | 0 | 0 | 0 |
| MAX | 77.2 | 95 | 96 | 98.7 | 51 | 96.3 | 94 | 99 | 99 | 97.7 | 80.2 | 95.6 | 95 |
| | 14 | 15 | 16 | 17 | 18 | 19 | 20 | 21 | 22 | 23 | 24 | 25 | 26 |
| | 27.2 | 0 | 0 | 0 | 0 | 0 | 0 | 0 | 0 | 0 | 1.01 | 1.01 | 0.34 |
| | 66.4 | 0.34 | 1.34 | 0.34 | 0 | 0 | 0 | 1.01 | 0.34 | 75.5 | 83.2 | 1.34 | 1.01 |
| | 0 | 99.3 | 89.9 | 0.67 | 0 | 0 | 0 | 0 | 0 | 0.67 | 2.35 | 0 | 86.6 |
| | 0 | 0 | 0 | 0 | 0.34 | 0 | 0 | 0 | 0 | 0 | 0 | 0 | 0 |
| | 0 | 0 | 0 | 0 | 0 | 0 | 0 | 0 | 0 | 0.34 | 0.34 | 0 | 0.67 |
| | 0 | 0 | 0 | 0 | 98.7 | 0.67 | 98 | 0.67 | 0 | 0.67 | 0.34 | 0 | 0 |
| | 1.01 | 0 | 0 | 0 | 0 | 0 | 1.34 | 0 | 0 | 7.72 | 7.72 | 0 | 1.01 |
| | 0 | 0 | 0 | 0 | 0 | 0 | 0 | 1.01 | 0 | 0 | 0.67 | 0 | 0 |
| | 0 | 0 | 0 | 0 | 0 | 0 | 0 | 0 | 0 | 0 | 0 | 0 | 0 |
| | 0 | 0 | 0 | 0 | 0 | 0 | 0 | 0 | 99 | 0.67 | 0 | 0 | 0 |
| | 0.67 | 0 | 0.34 | 98.3 | 0.67 | 1.01 | 0 | 95.3 | 0.67 | 2.01 | 0.34 | 97 | 0.67 |
| | 4.03 | 0 | 0 | 0.34 | 0 | 4.36 | 0 | 1.34 | 0 | 8.72 | 2.35 | 0.67 | 0.67 |
| | 0 | 0 | 0 | 0 | 0 | 0 | 0 | 0.34 | 0 | 0 | 0 | 0 | 0 |
| | 0.34 | 0 | 0.34 | 0 | 0 | 0 | 0 | 0 | 0 | 0.34 | 0 | 0 | 0.34 |
| | 0 | 0 | 0 | 0 | 0 | 0 | 0 | 0 | 0 | 0 | 0.34 | 0 | 0 |
| | 0 | 0 | 0.34 | 0 | 0 | 0 | 0 | 0.34 | 0 | 0 | 0 | 0 | 0 |
| | 0 | 0 | 0 | 0.34 | 0.34 | 90.3 | 0.67 | 0 | 0 | 0 | 0.34 | 0 | 2.35 |
| | 0 | 0 | 0.34 | 0 | 0 | 3.69 | 0 | 0 | 0 | 1.01 | 0.34 | 0 | 0 |
| | 0 | 0 | 6.04 | 0 | 0 | 0 | 0 | 0 | 0 | 0 | 0.67 | 0 | 0.34 |
| | 0.34 | 0.34 | 1.34 | 0 | 0 | 0 | 0 | 0 | 0 | 2.35 | 0 | 0 | 6.04 |
| | 0 | 0 | 0 | 0 | 0 | 0 | 0 | 0 | 0 | 0 | 0 | 0 | 0 |
| | 66.4 | 99.3 | 89.9 | 98.3 | 98.7 | 90.3 | 98 | 95.3 | 99 | 75.5 | 83.2 | 97 | 86.6 |

| | CDR1 | | | | | | | | | |
|---|---|---|---|---|---|---|---|---|---|---|
| | 1 | 2 | 3 | 4 | 5 | 6 | 7 | 8 | 9 | 10 |
| P | 0.34 | 3.36 | 1.01 | 1.34 | 3.02 | 1.01 | 3.02 | 0 | 0.34 | 0 |
| A | 1.01 | 4.03 | 2.01 | 2.68 | 2.68 | 3.02 | 33.2 | 1.01 | 0 | 1.34 |
| G | 4.03 | 1.01 | 2.35 | 5.03 | 3.02 | 1.34 | 4.36 | 0.34 | 0.34 | 0.34 |
| M | 0 | 1.01 | 0.34 | 0.34 | 2.35 | 0 | 0.34 | 0.34 | 0.34 | 0 |
| I | 3.02 | 19.8 | 4.36 | 0.67 | 14.4 | 2.01 | 3.02 | 0 | 0 | 0 |
| L | 3.69 | 1.68 | 10.7 | 0 | 4.36 | 4.03 | 1.34 | 0 | 0 | 0 |
| V | 1.34 | 1.01 | 4.03 | 0.67 | 2.01 | 0.67 | 8.72 | 0 | 0 | 0 |
| F | 15.8 | 1.34 | 51.3 | 1.68 | 3.02 | 2.35 | 1.68 | 0 | 0.34 | 0.67 |
| W | 0.34 | 0 | 0.67 | 0.34 | 0.67 | 1.01 | 0 | 0 | 0 | 0 |
| C | 0 | 0 | 0 | 0.67 | 0 | 0.67 | 5.7 | 0 | 0 | 0.34 |
| S | 19.5 | 4.7 | 8.39 | 52.3 | 17.8 | 5.03 | 7.05 | 0 | 0 | 0.67 |
| T | 1.34 | 55.7 | 1.34 | 5.7 | 11.4 | 1.34 | 8.05 | 0.67 | 0.34 | 0.34 |
| N | 3.36 | 0.67 | 0.67 | 4.7 | 9.4 | 15.4 | 3.69 | 0.34 | 0 | 0.34 |
| Q | 0 | 0.34 | 0.34 | 0.67 | 0.34 | 0.34 | 0 | 0 | 0 | 0 |
| Y | 9.73 | 0.67 | 6.71 | 1.01 | 6.38 | 48.7 | 5.37 | 1.01 | 2.35 | 0 |
| H | 1.34 | 0.34 | 1.34 | 0.67 | 1.34 | 2.01 | 2.01 | 0.67 | 0 | 0 |
| R | 33.2 | 1.34 | 1.01 | 9.4 | 7.72 | 1.34 | 2.01 | 0.34 | 0 | 0 |
| K | 0 | 0.34 | 0 | 0.34 | 0.67 | 0.67 | 0.34 | 0 | 0 | 0 |
| D | 1.68 | 2.35 | 2.68 | 10.7 | 6.04 | 4.7 | 6.38 | 0.67 | 0 | 0 |
| E | 0.34 | 0.34 | 0.67 | 1.01 | 1.01 | 1.34 | 0 | 0 | 0.34 | 0 |
| | 0 | 0 | 0 | 0 | 2.35 | 3.02 | 3.69 | 94.6 | 95.6 | 96 |
| | 33.2 | 55.7 | 51.3 | 52.3 | 17.8 | 48.7 | 33.2 | 1.01 | 2.35 | 1.34 |

| | Frame2 | | | | | | | | | |
|---|---|---|---|---|---|---|---|---|---|---|
| | 1 | 2 | 3 | 4 | 5 | 6 | 7 | 8 | 9 | 10 |
| P | 0 | 0 | 0 | 0 | 0 | 0 | 1.34 | 97 | 0.34 | 0.34 |
| A | 1.68 | 14.4 | 0 | 0 | 0 | 0 | 89.6 | 1.01 | 0 | 0.67 |
| G | 0.34 | 75.8 | 0 | 0 | 0 | 0 | 1.01 | 0 | 99 | 0.34 |
| M | 73.2 | 0.34 | 0 | 0 | 0 | 0 | 0 | 0 | 0 | 0.34 |
| I | 13.8 | 0 | 0 | 0 | 0 | 0 | 0.34 | 0 | 0 | 0 |
| L | 2.68 | 0 | 0 | 0.67 | 0 | 0 | 0.34 | 0.67 | 0 | 0.34 |
| V | 5.7 | 0 | 0 | 0.67 | 0 | 0 | 3.69 | 0 | 0 | 0 |
| F | 0 | 0 | 0 | 67.1 | 0.34 | 0 | 0 | 0 | 0 | 0 |
| W | 0.67 | 0 | 99.3 | 0.67 | 0 | 0 | 0 | 0 | 0 | 0 |
| C | 0.34 | 0.34 | 0 | 0 | 0 | 0 | 0 | 0 | 0 | 0 |
| S | 0.67 | 1.68 | 0 | 0 | 0 | 0 | 0.67 | 0.34 | 0 | 0.67 |
| T | 0.67 | 1.34 | 0 | 0 | 0 | 0 | 1.68 | 0.67 | 0 | 1.01 |
| N | 0 | 1.68 | 0 | 0 | 0 | 0 | 0 | 0 | 0 | 2.01 |
| Q | 0.34 | 0.34 | 0 | 0 | 0 | 95 | 0.34 | 0 | 0 | 1.34 |
| Y | 0 | 0 | 0.34 | 30.5 | 0 | 0 | 0 | 0 | 0 | 0 |
| H | 0 | 0 | 0 | 0 | 0.34 | 0 | 0 | 0 | 0 | 0 |
| R | 0 | 2.35 | 0.34 | 0 | 99.3 | 4.7 | 0.34 | 0.34 | 0 | 1.68 |
| K | 0 | 0 | 0 | 0 | 0 | 0.34 | 0 | 0 | 0 | 88.9 |
| D | 0 | 1.68 | 0 | 0.34 | 0 | 0 | 0.67 | 0 | 0 | 0 |
| E | 0 | 0 | 0 | 0 | 0 | 0 | 0 | 0 | 0.67 | 2.35 |
| | 0 | 0 | 0 | 0 | 0 | 0 | 0 | 0 | 0 | 0 |
| | 73.2 | 75.8 | 99.3 | 67.1 | 99.3 | 95 | 89.6 | 97 | 99 | 88.9 |

| 11 | 12 | 13 | 14 | 15 | 16 | 17 | 18 | 19 |
|---|---|---|---|---|---|---|---|---|
| 1.01 | 0.34 | 0 | 0.67 | 0 | 0 | 0 | 0 | 0.34 |
| 1.34 | 0 | 0.67 | 1.34 | 0 | 79.5 | 32.9 | 0.34 | 2.01 |
| 4.36 | 0.34 | 0.67 | 21.5 | 0 | 3.69 | 9.73 | 0.34 | 3.36 |
| 0 | 0 | 0 | 1.34 | 0 | 0 | 0 | 5.37 | 0.34 |
| 0 | 0 | 0.34 | 0 | 2.01 | 0 | 1.68 | 80.9 | 0.34 |
| 0.67 | 1.34 | 0 | 25.2 | 3.36 | 0.67 | 2.35 | 3.36 | 1.34 |
| 0 | 0 | 0.34 | 1.34 | 94 | 1.34 | 4.03 | 5.03 | 1.01 |
| 0 | 0 | 0 | 38.9 | 0 | 0 | 2.01 | 0.67 | 2.01 |
| 0 | 0 | 0 | 6.38 | 0 | 0 | 0 | 0 | 0 |
| 0 | 1.01 | 0 | 0 | 0 | 0 | 10.4 | 0 | 0 |
| 0.34 | 0 | 0 | 1.34 | 0.34 | 13.8 | 12.1 | 1.68 | 36.2 |
| 0 | 0.34 | 0 | 0 | 0 | 0.67 | 10.4 | 0.67 | 20.8 |
| 0 | 0.34 | 0 | 0 | 0 | 0 | 0.67 | 0.67 | 13.1 |
| 18.1 | 0 | 0.67 | 0 | 0 | 0 | 1.34 | 0 | 0 |
| 0 | 0.34 | 0 | 1.01 | 0 | 0 | 1.68 | 0 | 3.36 |
| 0.34 | 0.67 | 0 | 0 | 0 | 0 | 1.34 | 0 | 1.34 |
| 1.01 | 95.3 | 0 | 0.34 | 0 | 0 | 5.7 | 0.34 | 7.38 |
| 0.67 | 0 | 0 | 0.67 | 0 | 0 | 0.67 | 0.34 | 0.67 |
| 1.68 | 0 | 4.03 | 0 | 0.34 | 0 | 2.01 | 0 | 6.04 |
| 70.5 | 0 | 93.3 | 0 | 0 | 0.34 | 1.01 | 0.34 | 0.34 |
| 0 | 0 | 0 | 0 | 0 | 0 | 0 | 0 | 0 |
| 70.5 | 95.3 | 93.3 | 38.9 | 94 | 79.5 | 32.9 | 80.9 | 36.2 |

| | CDR2 | | | | | | | | | |
|---|---|---|---|---|---|---|---|---|---|---|
| | 1 | 2 | 3 | 4 | 5 | 6 | 7 | 8 | 9 | 10 |
| P | 2.01 | 0.34 | 1.01 | 0 | 0.34 | 0.34 | 0 | 0 | 0 | 0 |
| A | 3.69 | 4.7 | 2.35 | 4.36 | 1.01 | 0.34 | 0.67 | 0.34 | 0 | 0 |
| G | 5.7 | 23.5 | 69.5 | 37.2 | 6.71 | 0.67 | 1.01 | 0 | 0 | 0 |
| M | 0.67 | 0.34 | 0.34 | 1.01 | 1.01 | 0 | 0 | 0 | 0 | 0 |
| I | 1.34 | 2.01 | 0 | 3.02 | 2.68 | 0.67 | 0.34 | 0 | 0 | 0 |
| L | 1.01 | 1.68 | 0.67 | 0.67 | 3.02 | 0 | 0 | 0 | 0 | 0 |
| V | 1.34 | 3.02 | 0.67 | 3.36 | 2.01 | 0 | 0 | 0 | 0 | 0 |
| F | 1.01 | 1.68 | 1.01 | 0.34 | 0.34 | 0.67 | 0 | 0 | 0.34 | 0 |
| W | 21.8 | 0 | 0 | 0.34 | 0.34 | 0 | 0 | 0 | 0 | 0 |
| C | 0.34 | 0 | 0 | 0 | 0 | 0 | 0 | 0 | 0 | 0 |
| S | 30.9 | 32.6 | 5.7 | 16.4 | 20.8 | 0.67 | 0.34 | 0.34 | 0.34 | 0 |
| T | 7.05 | 5.7 | 3.36 | 7.05 | 8.72 | 0 | 0 | 0.34 | 0.34 | 0 |
| N | 4.03 | 5.37 | 2.01 | 3.36 | 6.38 | 0.34 | 0 | 0 | 0 | 0 |
| Q | 1.01 | 0.67 | 0 | 0.34 | 0.34 | 0 | 0 | 0 | 0 | 0 |
| Y | 0.34 | 2.01 | 0.67 | 1.68 | 2.68 | 0.34 | 0.67 | 0.34 | 0.34 | 0.67 |
| H | 0 | 1.01 | 0.34 | 0.67 | 0.34 | 0 | 0 | 0 | 0 | 0 |
| R | 13.8 | 5.37 | 2.35 | 7.05 | 5.37 | 0 | 0 | 0 | 0.34 | 0 |
| K | 1.01 | 1.01 | 0 | 1.68 | 1.01 | 0.34 | 0 | 0.34 | 0 | 0 |
| D | 2.01 | 7.38 | 9.73 | 8.39 | 2.01 | 0.34 | 0 | 0.67 | 0 | 0 |
| E | 1.01 | 1.68 | 0 | 1.01 | 0.67 | 0 | 0 | 0 | 0 | 0 |
| | 0 | 0 | 0.34 | 2.01 | 34.2 | 95.3 | 97 | 97.7 | 98.3 | 99.3 |
| | 30.9 | 32.6 | 69.5 | 37.2 | 20.8 | 0.67 | 1.01 | 0.67 | 0.34 | 0.67 |

| | Frame3 | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| P | 3 | 0 | 0 | 2 | 0 | 2 | 0 | 0 | 0 | 0 | 0 | 0 | 0 | 0 |
| A | 6 | 2 | 0 | 73 | 1 | 1 | 2 | 1 | 0 | 0 | 0 | 3 | 1 | 1 |
| G | 0 | 0 | 0 | 6 | 2 | 0 | 0 | 0 | 94 | 0 | 0 | 0 | 0 | 0 |
| M | 0 | 1 | 0 | 0 | 0 | 0 | 1 | 0 | 0 | 0 | 0 | 0 | 1 | 0 |
| I | 4 | 2 | 2 | 1 | 0 | 0 | 0 | 0 | 0 | 0 | 0 | 0 | 90 | 0 |
| L | 2 | 4 | 1 | 1 | 0 | 0 | 2 | 1 | 0 | 0 | 0 | 0 | 1 | 0 |
| V | 1 | 3 | 1 | 3 | 0 | 0 | 93 | 0 | 0 | 0 | 1 | 0 | 4 | 0 |
| F | 0 | 2 | 1 | 0 | 0 | 3 | 0 | 0 | 0 | 0 | 96 | 0 | 1 | 0 |
| W | 0 | 3 | 0 | 0 | 0 | 0 | 0 | 0 | 0 | 0 | 0 | 0 | 0 | 0 |
| C | 0 | 0 | 0 | 0 | 0 | 0 | 0 | 0 | 0 | 0 | 0 | 0 | 0 | 0 |
| S | 3 | 5 | 1 | 2 | 1 | 92 | 0 | 0 | 0 | 0 | 0 | 2 | 0 | 97 |
| T | 76 | 6 | 0 | 6 | 0 | 1 | 0 | 0 | 0 | 0 | 0 | 93 | 1 | 1 |
| N | 0 | 18 | 0 | 0 | 2 | 0 | 0 | 1 | 0 | 0 | 0 | 0 | 0 | 0 |
| Q | 1 | 0 | 0 | 1 | 0 | 0 | 0 | 2 | 0 | 0 | 0 | 0 | 0 | 0 |
| Y | 0 | 34 | 92 | 0 | 0 | 0 | 0 | 0 | 0 | 0 | 0 | 0 | 0 | 0 |
| H | 0 | 4 | 1 | 0 | 0 | 0 | 0 | 0 | 0 | 0 | 0 | 0 | 0 | 0 |
| R | 1 | 3 | 0 | 2 | 0 | 0 | 0 | 4 | 0 | 99 | 1 | 0 | 0 | 0 |
| K | 0 | 2 | 0 | 1 | 0 | 0 | 0 | 88 | 0 | 0 | 0 | 0 | 0 | 0 |
| D | 0 | 8 | 0 | 0 | 89 | 0 | 0 | 0 | 4 | 0 | 0 | 1 | 0 | 0 |
| E | 0 | 1 | 0 | 1 | 4 | 0 | 0 | 2 | 1 | 0 | 0 | 0 | 0 | 0 |
| | 0 | 0 | 0 | 0 | 0 | 0 | 0 | 0 | 0 | 0 | 0 | 0 | 0 | 0 |
| | 76 | 34 | 92 | 73 | 89 | 92 | 93 | 88 | 94 | 99 | 96 | 93 | 90 | 97 |
| | 15 | 16 | 17 | 18 | 19 | 20 | 21 | 22 | 23 | 24 | 25 | 26 | 27 | 28 |
| | 0 | 0 | 0 | 0 | 0 | 0 | 0 | 0 | 0 | 0 | 0 | 0 | 0 | 0 |
| | 1 | 0 | 0 | 87 | 3 | 0 | 4 | 5 | 1 | 0 | 0 | 0 | 0 | 0 |
| | 2 | 1 | 0 | 4 | 0 | 0 | 0 | 0 | 0 | 0 | 0 | 0 | 0 | 2 |
| | 0 | 0 | 0 | 0 | 1 | 0 | 5 | 4 | 0 | 0 | 0 | 97 | 0 | 0 |
| | 1 | 0 | 2 | 0 | 0 | 0 | 1 | 1 | 0 | 0 | 0 | 0 | 0 | 0 |
| | 1 | 0 | 0 | 0 | 0 | 0 | 1 | 9 | 0 | 99 | 2 | 2 | 0 | 0 |
| | 0 | 0 | 1 | 2 | 0 | 0 | 0 | 80 | 0 | 0 | 0 | 0 | 0 | 1 |
| | 0 | 0 | 1 | 0 | 0 | 0 | 0 | 0 | 2 | 0 | 0 | 0 | 0 | 0 |
| | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 |
| | 2 | 0 | 4 | 1 | 1 | 2 | 1 | 0 | 5 | 0 | 0 | 0 | 4 | 82 |
| | 0 | 0 | 1 | 1 | 0 | 3 | 84 | 0 | 2 | 0 | 1 | 0 | 1 | 1 |
| | 0 | 1 | 79 | 1 | 2 | 84 | 0 | 0 | 1 | 0 | 0 | 0 | 84 | 12 |
| | 9 | 0 | 0 | 0 | 1 | 0 | 0 | 0 | 0 | 0 | 90 | 0 | 0 | 0 |
| | 0 | 0 | 1 | 0 | 0 | 0 | 0 | 0 | 83 | 0 | 0 | 0 | 0 | 0 |
| | 0 | 0 | 1 | 0 | 0 | 0 | 0 | 0 | 1 | 0 | 1 | 0 | 1 | 0 |
| | 82 | 0 | 0 | 0 | 3 | 1 | 1 | 0 | 0 | 0 | 2 | 0 | 0 | 1 |
| | 3 | 0 | 5 | 0 | 84 | 5 | 0 | 0 | 0 | 0 | 0 | 0 | 0 | 0 |
| | 0 | 97 | 5 | 3 | 0 | 2 | 0 | 0 | 3 | 0 | 1 | 0 | 9 | 1 |
| | 0 | 1 | 1 | 0 | 5 | 0 | 0 | 0 | 0 | 0 | 4 | 0 | 0 | 0 |
| | 0 | 0 | 0 | 0 | 0 | 0 | 0 | 0 | 0 | 0 | 0 | 0 | 0 | 0 |
| | 82 | 97 | 79 | 87 | 84 | 84 | 84 | 80 | 83 | 99 | 90 | 97 | 84 | 82 |

| 29 | 30 | 31 | 32 | 33 | 34 | 35 | 36 | 37 | 38 | 39 | 40 | 41 |
|---|---|---|---|---|---|---|---|---|---|---|---|---|
| 0 | 0 | 90 | 0 | 0 | 0 | 0 | 0 | 0 | 0 | 0 | 0 | 0 |
| 0 | 1 | 1 | 0 | 0 | 1 | 96 | 1 | 0 | 0 | 0 | 64 | 69 |
| 0 | 0 | 0 | 1 | 0 | 0 | 3 | 0 | 0 | 0 | 0 | 3 | 3 |
| 0 | 0 | 0 | 0 | 0 | 0 | 0 | 5 | 0 | 0 | 0 | 0 | 0 |
| 0 | 0 | 0 | 0 | 0 | 0 | 0 | 14 | 0 | 0 | 0 | 0 | 1 |
| 97 | 0 | 3 | 0 | 0 | 0 | 0 | 2 | 0 | 1 | 0 | 0 | 4 |
| 2 | 0 | 0 | 0 | 0 | 0 | 0 | 68 | 0 | 2 | 0 | 2 | 7 |
| 0 | 0 | 0 | 0 | 0 | 0 | 0 | 0 | 0 | 4 | 0 | 1 | 0 |
| 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 99 | 0 | 0 |
| 0 | 0 | 2 | 0 | 0 | 1 | 0 | 1 | 0 | 2 | 0 | 1 | 2 |
| 0 | 1 | 1 | 0 | 0 | 98 | 0 | 5 | 0 | 2 | 0 | 2 | 6 |
| 0 | 2 | 0 | 0 | 0 | 0 | 0 | 1 | 0 | 0 | 0 | 17 | 0 |
| 0 | 1 | 0 | 0 | 0 | 0 | 0 | 0 | 0 | 0 | 0 | 0 | 0 |
| 0 | 0 | 1 | 0 | 0 | 0 | 0 | 0 | 100 | 87 | 0 | 1 | 2 |
| 0 | 0 | 0 | 0 | 0 | 0 | 0 | 0 | 0 | 1 | 0 | 4 | 0 |
| 0 | 4 | 1 | 0 | 0 | 0 | 0 | 1 | 0 | 0 | 0 | 1 | 4 |
| 0 | 87 | 0 | 0 | 0 | 0 | 0 | 0 | 0 | 0 | 0 | 2 | 0 |
| 0 | 0 | 0 | 4 | 99 | 0 | 0 | 1 | 0 | 0 | 0 | 0 | 1 |
| 0 | 4 | 0 | 95 | 0 | 0 | 0 | 0 | 0 | 0 | 0 | 0 | 1 |
| 0 | 0 | 0 | 0 | 0 | 0 | 0 | 0 | 0 | 0 | 0 | 0 | 0 |
| 97 | 87 | 90 | 95 | 99 | 98 | 96 | 68 | 100 | 87 | 99 | 64 | 69 |

| | CDR3 | | | | | | | | | | | | | | |
|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 |
| P | 1.01 | 6.71 | 2.68 | 6.04 | 4.03 | 3.02 | 4.7 | 6.04 | 5.03 | 6.38 | 3.69 | 1.34 | 1.34 | 0.67 | 1.68 |
| A | 6.71 | 5.7 | 5.7 | 7.38 | 5.37 | 6.38 | 6.38 | 3.02 | 6.04 | 7.05 | 5.37 | 2.68 | 3.02 | 2.01 | 2.01 |
| G | 15.4 | 7.72 | 15.8 | 14.4 | 12.1 | 15.4 | 10.1 | 6.38 | 5.7 | 5.37 | 6.38 | 5.7 | 5.03 | 2.68 | 1.68 |
| M | 0 | 1.34 | 0 | 1.68 | 1.01 | 2.01 | 0.67 | 1.34 | 1.34 | 0.34 | 0.34 | 1.68 | 0.67 | 1.01 | 0 |
| I | 1.34 | 4.03 | 2.35 | 3.69 | 3.02 | 2.35 | 3.69 | 3.02 | 1.68 | 0.67 | 3.36 | 1.01 | 1.01 | 0.67 | 1.01 |
| L | 2.35 | 9.06 | 4.36 | 5.03 | 6.71 | 4.36 | 7.72 | 5.7 | 4.03 | 4.03 | 2.68 | 3.02 | 0.34 | 1.34 | 1.68 |
| V | 3.69 | 6.71 | 5.03 | 6.71 | 6.04 | 5.7 | 4.7 | 4.7 | 3.69 | 3.36 | 2.35 | 2.01 | 2.68 | 0.67 | 0.67 |
| F | 2.01 | 3.36 | 4.03 | 4.36 | 4.03 | 2.35 | 1.34 | 2.68 | 2.68 | 1.01 | 1.34 | 1.01 | 1.01 | 2.68 | 0.67 |
| W | 0.67 | 3.69 | 3.36 | 3.02 | 5.03 | 4.03 | 3.02 | 2.68 | 1.68 | 2.68 | 2.01 | 1.34 | 1.01 | 1.01 | 0.34 |
| C | 0.67 | 1.34 | 0.34 | 2.35 | 1.34 | 3.02 | 1.01 | 3.02 | 2.35 | 1.01 | 2.01 | 0.34 | 0 | 0.67 | 0.67 |
| S | 8.05 | 8.05 | 7.38 | 7.72 | 12.4 | 9.73 | 15.1 | 10.4 | 10.4 | 8.05 | 5.37 | 5.7 | 2.35 | 3.69 | 2.35 |
| T | 8.72 | 7.05 | 6.71 | 9.06 | 7.72 | 7.38 | 6.04 | 6.04 | 7.72 | 5.37 | 2.68 | 4.03 | 4.36 | 0.67 | 1.01 |
| N | 4.7 | 2.68 | 3.69 | 1.68 | 2.68 | 1.34 | 0 | 2.68 | 1.68 | 3.02 | 3.02 | 2.35 | 3.36 | 2.35 | 1.34 |
| Q | 2.01 | 1.34 | 5.03 | 1.68 | 1.68 | 1.01 | 1.01 | 1.01 | 1.68 | 1.01 | 2.68 | 1.68 | 1.01 | 0.67 | 0 |
| Y | 2.01 | 3.69 | 10.4 | 8.39 | 7.05 | 8.72 | 9.73 | 8.05 | 9.73 | 12.1 | 6.38 | 8.72 | 10.7 | 6.38 | 6.04 |
| H | 1.34 | 1.01 | 1.01 | 2.01 | 1.34 | 0 | 0.67 | 1.01 | 0.34 | 1.68 | 1.01 | 1.68 | 1.01 | 0.67 | 0 |
| R | 13.8 | 13.8 | 12.1 | 8.39 | 6.71 | 7.38 | 6.71 | 8.72 | 4.7 | 5.03 | 5.37 | 5.37 | 3.36 | 2.68 | 0.67 |
| K | 6.04 | 3.02 | 2.35 | 0 | 0.67 | 0.67 | 0 | 0 | 1.01 | 1.01 | 1.68 | 1.34 | 1.34 | 1.01 | 0.67 |
| D | 15.4 | 5.03 | 3.69 | 1.68 | 3.02 | 4.36 | 6.04 | 6.04 | 5.7 | 4.03 | 4.36 | 4.7 | 3.69 | 2.68 | 2.01 |
| E | 4.03 | 3.36 | 2.01 | 1.68 | 1.68 | 2.01 | 0.67 | 1.68 | 3.36 | 3.36 | 4.36 | 3.36 | 2.01 | 2.01 | 2.68 |
| | 0 | 1.34 | 2.01 | 3.02 | 6.38 | 8.72 | 10.7 | 15.8 | 19.5 | 23.5 | 33.6 | 40.9 | 50.7 | 63.8 | 72.8 |
| | 15.4 | 13.8 | 15.8 | 14.4 | 12.4 | 15.4 | 15.1 | 10.4 | 10.4 | 12.1 | 6.38 | 8.72 | 10.7 | 6.38 | 6.04 |
| | 16 | 17 | 18 | 19 | 20 | 21 | 22 | 23 | 24 | 25 | 26 | 27 | 28 | 29 | 30 |
| | 0.67 | 1.34 | 0 | 0.34 | 0.34 | 0 | 0 | 0 | 0 | 0 | 0 | 0 | 0 | 0 | 0 |
| | 0.34 | 0.34 | 1.01 | 0 | 0 | 0.34 | 0 | 0.34 | 0 | 0 | 0 | 0 | 0 | 0 | 0 |
| | 2.01 | 1.01 | 1.01 | 1.34 | 0 | 1.01 | 0 | 0.34 | 0.34 | 0 | 0 | 0 | 0 | 0 | 0 |
| | 0 | 0 | 0.67 | 0 | 0 | 0.34 | 0.67 | 0.34 | 0 | 0 | 0 | 0 | 0 | 0 | 0 |
| | 0 | 0.34 | 0 | 0 | 0.34 | 0.34 | 0.34 | 0 | 0 | 0 | 0 | 0 | 0 | 0 | 0.34 |
| | 2.01 | 1.34 | 0.34 | 0 | 0 | 0 | 0 | 0 | 0 | 0 | 0 | 0 | 0 | 0 | 0 |
| | 0.67 | 0 | 0 | 0 | 0.67 | 0.34 | 0 | 0 | 0 | 0 | 0 | 0 | 0 | 0 | 0 |
| | 0.34 | 0.67 | 0.34 | 0.34 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0.34 | 0.34 | 0 | 0 |
| | 0.34 | 0 | 0 | 0 | 0 | 0 | 0 | 0 | 0 | 0 | 0 | 0 | 0 | 0 | 0 |
| | 1.34 | 1.01 | 0.34 | 0.34 | 0.34 | 0 | 0 | 0 | 0 | 0 | 0 | 0 | 0 | 0.34 | 0.34 |
| | 1.68 | 1.01 | 0 | 0 | 0 | 0.34 | 0 | 0 | 0 | 0 | 0.34 | 0.34 | 0 | 0 | 0 |
| | 0.34 | 1.01 | 0.34 | 0 | 0 | 0 | 0 | 0 | 0 | 0 | 0 | 0 | 0 | 0 | 0 |
| | 0.67 | 0 | 0 | 0 | 0 | 0 | 0 | 0 | 0 | 0 | 0 | 0 | 0 | 0 | 0 |
| | 8.39 | 3.02 | 2.68 | 1.01 | 1.34 | 0 | 1.01 | 0 | 0.34 | 0 | 0 | 0 | 0 | 0 | 0 |
| | 0 | 0.34 | 0 | 0 | 0 | 0 | 0 | 0 | 0 | 0.34 | 0.34 | 0 | 0 | 0 | 0 |
| | 0.67 | 0.34 | 0 | 0.34 | 0 | 0 | 0 | 0 | 0.34 | 0.34 | 0 | 0 | 0 | 0 | 0 |
| | 0 | 0 | 0.67 | 0.34 | 0 | 0 | 0.34 | 0 | 0 | 0 | 0 | 0 | 0 | 0 | 0 |
| | 1.01 | 0.67 | 0.67 | 0 | 0.34 | 0 | 0 | 0 | 0 | 0 | 0 | 0 | 0 | 0 | 0 |
| | 0.67 | 0 | 0 | 0 | 0 | 0 | 0 | 0 | 0 | 0 | 0 | 0 | 0.34 | 0.34 | 0 |
| | 78.9 | 87.6 | 91.9 | 96 | 96.6 | 97.3 | 97.7 | 99 | 99 | 99.3 | 99.3 | 99.3 | 99.3 | 99.3 | 99.3 |
| | 8.39 | 3.02 | 2.68 | 1.34 | 1.34 | 1.01 | 1.01 | 0.34 | 0.34 | 0.34 | 0.34 | 0.34 | 0.34 | 0.34 | 0.34 |

VHH input library (43105 VHHs)

| | Frame4 | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 13 |
| P | 2.35 | 1.01 | 0 | 0 | 1.01 | 0 | 0 | 1.34 | 0 | 0 | 0 | 0 | 0 |
| A | 4.36 | 1.01 | 0.34 | 0 | 0 | 0 | 0.34 | 0 | 0 | 0 | 0 | 0 | 1.68 |
| G | 8.05 | 0.67 | 0 | 100 | 0 | 100 | 0 | 0 | 0 | 0 | 0 | 0 | 0 |
| M | 0.67 | 0 | 0 | 0 | 0 | 0 | 0 | 0 | 0 | 0 | 0 | 0 | 0 |
| I | 2.01 | 1.34 | 0 | 0 | 0 | 0 | 0 | 0 | 0 | 0 | 0.67 | 0 | 0 |
| L | 0.67 | 1.68 | 0 | 0 | 0.34 | 0 | 0 | 2.35 | 0 | 0 | 0 | 0 | 0.34 |
| V | 1.68 | 2.68 | 0 | 0 | 0 | 0 | 0 | 0 | 100 | 0 | 99.3 | 0.34 | 0 |
| F | 0.34 | 3.69 | 0 | 0 | 0 | 0 | 0 | 0 | 0 | 0 | 0 | 0 | 0 |
| W | 0.67 | 0.34 | 97 | 0 | 0 | 0 | 0 | 0 | 0 | 0 | 0 | 0 | 0 |
| C | 0 | 0 | 0 | 0 | 0 | 0 | 0 | 0 | 0 | 0 | 0 | 0 | 0 |
| S | 4.03 | 7.38 | 0.34 | 0 | 0 | 0 | 0 | 0 | 0 | 0 | 0 | 99 | 92.3 |
| T | 3.69 | 1.68 | 0 | 0 | 0 | 0 | 99 | 0.34 | 0 | 100 | 0 | 0 | 0 |
| N | 11.1 | 1.01 | 0 | 0 | 0 | 0 | 0 | 0 | 0 | 0 | 0 | 0 | 0 |
| Q | 0.34 | 0.67 | 0 | 0 | 90.3 | 0 | 0 | 94 | 0 | 0 | 0 | 0 | 0 |
| Y | 1.01 | 67.8 | 0.67 | 0 | 0 | 0 | 0 | 0 | 0 | 0 | 0 | 0 | 0 |
| H | 2.01 | 5.37 | 0 | 0 | 0.34 | 0 | 0 | 0 | 0 | 0 | 0 | 0 | 0.34 |
| R | 3.02 | 0.67 | 1.68 | 0 | 2.68 | 0 | 0 | 1.68 | 0 | 0 | 0 | 0 | 0 |
| K | 1.34 | 0.34 | 0 | 0 | 5.37 | 0 | 0 | 0.34 | 0 | 0 | 0 | 0 | 0.34 |
| D | 46 | 2.68 | 0 | 0 | 0 | 0 | 0 | 0 | 0 | 0 | 0 | 0 | 0 |
| E | 6.71 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 5.03 |
| | 46 | 67.8 | 97 | 100 | 90.3 | 100 | 99 | 14 | 100 | 100 | 99.3 | 99 | 92.3 |

| | Frame1 | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 13 |
| P | 0 | 0 | 0 | 0 | 0 | 0 | 0 | 0 | 0 | 0 | 0 | 0 | 0 |
| A | 0 | 0 | 0 | 0 | 0 | 0 | 0 | 0 | 0 | 32 | 0 | 0 | 0 |
| G | 0 | 0 | 0 | 0 | 0 | 0 | 0 | 99 | 99 | 67 | 0 | 0 | 0 |
| M | 0 | 0 | 0 | 0 | 0 | 0 | 0 | 0 | 0 | 0 | 0 | 0 | 0 |
| I | 0 | 0 | 0 | 0 | 0 | 0 | 0 | 0 | 0 | 0 | 0 | 0 | 0 |
| L | 0 | 0 | 0 | 99 | 0 | 0 | 0 | 0 | 0 | 0 | 99 | 0 | 0 |
| V | 0 | 99 | 0 | 0 | 69 | 0 | 0 | 0 | 0 | 0 | 0 | 99 | 0 |
| F | 0 | 0 | 0 | 0 | 1 | 0 | 0 | 0 | 0 | 0 | 0 | 0 | 0 |
| W | 0 | 0 | 0 | 0 | 0 | 0 | 0 | 0 | 0 | 0 | 0 | 0 | 0 |
| C | 0 | 0 | 0 | 0 | 0 | 0 | 0 | 0 | 0 | 0 | 0 | 0 | 0 |
| S | 0 | 0 | 0 | 0 | 0 | 0 | 99 | 0 | 0 | 0 | 0 | 0 | 0 |
| T | 0 | 0 | 0 | 0 | 0 | 0 | 0 | 0 | 0 | 0 | 0 | 0 | 0 |
| N | 0 | 0 | 0 | 0 | 0 | 0 | 0 | 0 | 0 | 0 | 0 | 0 | 0 |
| Q | 63 | 0 | 99 | 0 | 30 | 0 | 0 | 0 | 0 | 0 | 0 | 0 | 98 |
| Y | 0 | 0 | 0 | 0 | 0 | 0 | 0 | 0 | 0 | 0 | 0 | 0 | 0 |
| H | 0 | 0 | 0 | 0 | 0 | 0 | 0 | 0 | 0 | 0 | 0 | 0 | 2 |
| R | 0 | 0 | 0 | 0 | 0 | 0 | 0 | 0 | 0 | 0 | 0 | 0 | 0 |
| K | 0 | 0 | 0 | 0 | 0 | 0 | 0 | 0 | 0 | 0 | 0 | 0 | 0 |
| D | 0 | 0 | 0 | 0 | 0 | 1 | 0 | 0 | 0 | 0 | 0 | 0 | 0 |
| E | 37 | 0 | 0 | 0 | 0 | 99 | 0 | 0 | 0 | 0 | 0 | 0 | 0 |
| MAX | 0 | 0 | 0 | 0 | 0 | 0 | 0 | 0 | 0 | 0 | 0 | 0 | 0 |
| | 63 | 99 | 99 | 99 | 69 | 99 | 99 | 99 | 99 | 67 | 99 | 99 | 98 |
| | 14 | 15 | 16 | 17 | 18 | 19 | 20 | 21 | 22 | 23 | 24 | 25 | 26 |
| | 32 | 0 | 0 | 0 | 0 | 0 | 0 | 0 | 0 | 0 | 0 | 0 | 0 |
| | 67 | 0 | 0 | 0 | 0 | 0 | 0 | 0 | 0 | 89 | 98 | 0 | 0 |
| | 0 | 99 | 63 | 0 | 0 | 0 | 0 | 0 | 0 | 0 | 0 | 0 | 97 |
| | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 |
| | 0 | 0 | 0 | 0 | 99 | 0 | 99 | 0 | 0 | 0 | 0 | 0 | 0 |
| | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 |
| | 0 | 0 | 0 | 0 | 0 | 0 | 0 | 0 | 99 | 0 | 0 | 0 | 0 |
| | 0 | 0 | 0 | 98 | 0 | 0 | 0 | 99 | 0 | 0 | 0 | 98 | 0 |
| | 0 | 0 | 0 | 0 | 0 | 0 | 0 | 0 | 0 | 10 | 0 | 0 | 0 |
| | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 |
| | 0 | 0 | 0 | 2 | 0 | 99 | 0 | 0 | 0 | 0 | 0 | 0 | 0 |
| | 0 | 0 | 0 | 0 | 0 | 0 | 0 | 0 | 0 | 0 | 0 | 0 | 0 |
| | 0 | 0 | 37 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 |
| | 67 | 99 | 63 | 98 | 99 | 99 | 99 | 99 | 99 | 89 | 98 | 98 | 97 |

| | CDR1 | | | | | | | | | |
|---|---|---|---|---|---|---|---|---|---|---|
| | 1 | 2 | 3 | 4 | 5 | 6 | 7 | 8 | 9 | 10 |
| P | 3 | 3 | 3 | 3 | 2 | 2 | 2 | 0 | 0 | 0 |
| A | 5 | 5 | 5 | 5 | 5 | 5 | 6 | 0 | 0 | 0 |
| G | 9 | 7 | 8 | 8 | 8 | 8 | 8 | 0 | 0 | 0 |
| M | 3 | 3 | 2 | 2 | 3 | 2 | 2 | 0 | 0 | 0 |
| I | 4 | 5 | 4 | 4 | 4 | 4 | 4 | 0 | 0 | 0 |
| L | 8 | 8 | 8 | 8 | 8 | 7 | 7 | 0 | 0 | 0 |
| V | 9 | 8 | 10 | 9 | 9 | 9 | 9 | 0 | 0 | 0 |
| F | 4 | 6 | 7 | 5 | 5 | 4 | 4 | 0 | 0 | 0 |
| W | 2 | 3 | 3 | 3 | 3 | 3 | 2 | 0 | 0 | 0 |
| C | 6 | 6 | 6 | 5 | 5 | 5 | 5 | 0 | 0 | 0 |
| S | 9 | 9 | 8 | 10 | 10 | 8 | 8 | 0 | 0 | 0 |
| T | 4 | 6 | 4 | 4 | 4 | 4 | 4 | 0 | 0 | 0 |
| N | 4 | 3 | 3 | 3 | 2 | 2 | 2 | 0 | 0 | 0 |
| Q | 2 | 1 | 1 | 1 | 1 | 1 | 1 | 0 | 0 | 0 |
| Y | 5 | 5 | 4 | 4 | 4 | 5 | 4 | 0 | 0 | 0 |
| H | 4 | 3 | 3 | 3 | 2 | 2 | 2 | 0 | 0 | 0 |
| R | 8 | 7 | 7 | 7 | 7 | 7 | 7 | 0 | 0 | 0 |
| K | 2 | 2 | 2 | 2 | 2 | 2 | 1 | 0 | 0 | 0 |
| D | 3 | 3 | 3 | 3 | 3 | 3 | 3 | 0 | 0 | 0 |
| E | 2 | 2 | 2 | 2 | 2 | 2 | 2 | 0 | 0 | 0 |
| | 3 | 6 | 8 | 10 | 13 | 14 | 17 | 99 | 99 | 99 |
| | 9 | 9 | 10 | 10 | 10 | 9 | 9 | 0 | 0 | 0 |

| | Frame2 | | | | | | | | | |
|---|---|---|---|---|---|---|---|---|---|---|
| | 1 | 2 | 3 | 4 | 5 | 6 | 7 | 8 | 9 | 10 |
| P | 0 | 0 | 0 | 0 | 0 | 0 | 0 | 99 | 0 | 0 |
| A | 0 | 0 | 0 | 0 | 0 | 0 | 99 | 0 | 0 | 0 |
| G | 1 | 99 | 0 | 0 | 0 | 0 | 0 | 0 | 99 | 0 |
| M | 98 | 0 | 0 | 0 | 0 | 0 | 0 | 0 | 0 | 0 |
| I | 0 | 0 | 0 | 0 | 0 | 0 | 0 | 0 | 0 | 0 |
| L | 0 | 0 | 0 | 0 | 0 | 0 | 0 | 0 | 0 | 0 |
| V | 0 | 0 | 0 | 0 | 0 | 0 | 0 | 0 | 0 | 0 |
| F | 0 | 0 | 0 | 78 | 0 | 0 | 0 | 0 | 0 | 0 |
| W | 0 | 0 | 99 | 0 | 0 | 0 | 0 | 0 | 0 | 0 |
| C | 0 | 0 | 0 | 0 | 0 | 0 | 0 | 0 | 0 | 0 |
| S | 0 | 0 | 0 | 0 | 0 | 0 | 0 | 0 | 0 | 0 |
| T | 0 | 0 | 0 | 0 | 0 | 0 | 0 | 0 | 0 | 0 |
| N | 0 | 0 | 0 | 0 | 0 | 0 | 0 | 0 | 0 | 0 |
| Q | 0 | 0 | 0 | 0 | 0 | 99 | 0 | 0 | 0 | 0 |
| Y | 0 | 0 | 0 | 22 | 0 | 0 | 0 | 0 | 0 | 0 |
| H | 0 | 0 | 0 | 0 | 0 | 0 | 0 | 0 | 0 | 0 |
| R | 0 | 0 | 0 | 0 | 99 | 0 | 0 | 0 | 0 | 0 |
| K | 0 | 0 | 0 | 0 | 0 | 0 | 0 | 0 | 0 | 99 |
| D | 0 | 0 | 0 | 0 | 0 | 0 | 0 | 0 | 0 | 0 |
| E | 0 | 0 | 0 | 0 | 0 | 0 | 0 | 0 | 0 | 0 |
| | 0 | 0 | 0 | 0 | 0 | 0 | 0 | 0 | 0 | 0 |
| | 98 | 99 | 99 | 78 | 99 | 99 | 99 | 99 | 99 | 99 |

| 11 | 12 | 13 | 14 | 15 | 16 | 17 | 18 | 19 |
|---|---|---|---|---|---|---|---|---|
| 0 | 0 | 0 | 0 | 0 | 0 | 0 | 0 | 0 |
| 0 | 0 | 0 | 0 | 0 | 99 | 30 | 0 | 0 |
| 0 | 0 | 0 | 0 | 0 | 0 | 0 | 0 | 0 |
| 0 | 0 | 0 | 0 | 0 | 0 | 0 | 0 | 0 |
| 0 | 0 | 0 | 0 | 0 | 0 | 0 | 98 | 0 |
| 0 | 0 | 0 | 0 | 0 | 0 | 0 | 0 | 0 |
| 0 | 0 | 0 | 0 | 99 | 0 | 0 | 0 | 0 |
| 0 | 0 | 0 | 99 | 0 | 0 | 0 | 0 | 0 |
| 0 | 0 | 0 | 0 | 0 | 0 | 0 | 0 | 0 |
| 0 | 0 | 0 | 0 | 0 | 0 | 0 | 0 | 0 |
| 0 | 0 | 0 | 0 | 0 | 0 | 30 | 0 | 28 |
| 0 | 0 | 0 | 0 | 0 | 0 | 38 | 0 | 34 |
| 0 | 0 | 0 | 0 | 0 | 0 | 0 | 0 | 36 |
| 0 | 0 | 0 | 0 | 0 | 0 | 0 | 0 | 0 |
| 0 | 0 | 0 | 0 | 0 | 0 | 0 | 0 | 0 |
| 0 | 0 | 0 | 0 | 0 | 0 | 0 | 0 | 0 |
| 0 | 99 | 0 | 0 | 0 | 0 | 0 | 0 | 0 |
| 0 | 0 | 0 | 0 | 0 | 0 | 0 | 0 | 0 |
| 0 | 0 | 0 | 0 | 0 | 0 | 0 | 0 | 0 |
| 99 | 0 | 99 | 0 | 0 | 0 | 0 | 0 | 0 |
| 0 | 0 | 0 | 0 | 0 | 0 | 0 | 0 | 0 |
| 99 | 99 | 99 | 99 | 99 | 99 | 3 | 98 | 36 |

| | CDR2 | | | | | | | | | |
|---|---|---|---|---|---|---|---|---|---|---|
| | 1 | 2 | 3 | 4 | 5 | 6 | 7 | 8 | 9 | 10 |
| P | 4 | 4 | 4 | 4 | 3 | 0 | 0 | 0 | 0 | 0 |
| A | 4 | 6 | 6 | 6 | 6 | 0 | 0 | 0 | 0 | 0 |
| G | 7 | 7 | 8 | 9 | 9 | 0 | 0 | 0 | 0 | 0 |
| M | 3 | 2 | 2 | 2 | 2 | 0 | 0 | 0 | 0 | 0 |
| I | 5 | 4 | 4 | 3 | 4 | 0 | 0 | 0 | 0 | 0 |
| L | 10 | 9 | 8 | 9 | 8 | 0 | 0 | 0 | 0 | 0 |
| V | 8 | 9 | 8 | 8 | 9 | 0 | 0 | 0 | 0 | 0 |
| F | 7 | 5 | 5 | 5 | 5 | 0 | 0 | 0 | 0 | 0 |
| W | 3 | 3 | 3 | 3 | 3 | 0 | 0 | 0 | 0 | 0 |
| C | 6 | 6 | 6 | 6 | 6 | 0 | 0 | 0 | 0 | 0 |
| S | 10 | 10 | 10 | 9 | 9 | 0 | 0 | 0 | 0 | 0 |
| T | 5 | 5 | 5 | 4 | 4 | 0 | 0 | 0 | 0 | 0 |
| N | 3 | 3 | 3 | 3 | 3 | 0 | 0 | 0 | 0 | 0 |
| Q | 2 | 2 | 2 | 2 | 1 | 0 | 0 | 0 | 0 | 0 |
| Y | 4 | 5 | 4 | 4 | 4 | 0 | 0 | 0 | 0 | 0 |
| H | 4 | 3 | 3 | 3 | 3 | 0 | 0 | 0 | 0 | 0 |
| R | 8 | 8 | 8 | 8 | 8 | 0 | 0 | 0 | 0 | 0 |
| K | 1 | 1 | 1 | 1 | 1 | 0 | 0 | 0 | 0 | 0 |
| D | 3 | 3 | 4 | 3 | 4 | 0 | 0 | 0 | 0 | 0 |
| E | 1 | 2 | 2 | 2 | 2 | 0 | 0 | 0 | 0 | 0 |
| | 2 | 3 | 4 | 5 | 6 | 99 | 99 | 99 | 99 | 99 |
| | 10 | 10 | 10 | 9 | 9 | 0 | 0 | 0 | 0 | 0 |

| | Frame3 | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| P | 0 | 0 | 0 | 0 | 0 | 0 | 0 | 0 | 0 | 0 | 0 | 0 | 0 | 0 |
| A | 0 | 0 | 0 | 99 | 0 | 0 | 0 | 0 | 0 | 0 | 0 | 0 | 0 | 0 |
| G | 0 | 0 | 0 | 0 | 0 | 0 | 0 | 0 | 99 | 0 | 0 | 0 | 0 | 0 |
| M | 0 | 0 | 0 | 0 | 0 | 0 | 0 | 0 | 0 | 0 | 0 | 0 | 0 | 0 |
| I | 0 | 0 | | 0 | 0 | 0 | 0 | 0 | 0 | 0 | 0 | 0 | 99 | 0 |
| L | 0 | 0 | 0 | 0 | 0 | 0 | 0 | 0 | 0 | 0 | 0 | 0 | 0 | 0 |
| V | 0 | 0 | 0 | 0 | 0 | 0 | 99 | 0 | 0 | 0 | 0 | 0 | 0 | 0 |
| F | 0 | 0 | 0 | 0 | 0 | 0 | 0 | 0 | 0 | 0 | 99 | 0 | 0 | 0 |
| W | 0 | 0 | 0 | 0 | 0 | 0 | 0 | 0 | 0 | 0 | 0 | 0 | 0 | 0 |
| C | 0 | 0 | 0 | 0 | 0 | 0 | 0 | 0 | 0 | 0 | 0 | 0 | 0 | 0 |
| S | 0 | 0 | 0 | 0 | 0 | 99 | 0 | 0 | 0 | 0 | 0 | 34 | 0 | 99 |
| T | 99 | 0 | 0 | 0 | 0 | 0 | 0 | 0 | 0 | 0 | 0 | 66 | 0 | 0 |
| N | 0 | 25 | 0 | 0 | 0 | 0 | 0 | 0 | 0 | 0 | 0 | 0 | 0 | 0 |
| Q | 0 | 0 | 0 | 0 | 0 | 0 | 0 | 0 | 0 | 0 | 0 | 0 | 0 | 0 |
| Y | 0 | 36 | 99 | 0 | 0 | 0 | 0 | 0 | 0 | 0 | 0 | 0 | 0 | 0 |
| H | 0 | 0 | 0 | 0 | 0 | 0 | 0 | 0 | 0 | 0 | 0 | 0 | 0 | 0 |
| R | 0 | 0 | 0 | 0 | 0 | 0 | 0 | 34 | 0 | 99 | 0 | 0 | 0 | 0 |
| K | 0 | 0 | 0 | 0 | 0 | 0 | 0 | 66 | 0 | 0 | 0 | 0 | 0 | 0 |
| D | 0 | 39 | 0 | 0 | 100 | 0 | 0 | 0 | 0 | 0 | 0 | 0 | 0 | 0 |
| E | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 |
| | 99 | 39 | 99 | 99 | 100 | 99 | 99 | 66 | 99 | 99 | 99 | 66 | 99 | 99 |
| | 15 | 16 | 17 | 18 | 19 | 20 | 21 | 22 | 23 | 24 | 25 | 26 | 27 | 28 |
| | 0 | 0 | 0 | 0 | 0 | 0 | 0 | 0 | 0 | 0 | 0 | 0 | 0 | 0 |
| | 33 | 0 | 0 | 99 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 99 | 0 | 0 |
| | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 99 | 0 | 0 | 0 | 0 |
| | 0 | 0 | 0 | 0 | 0 | 0 | 0 | 99 | 0 | 0 | 0 | 0 | 0 | 0 |
| | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 |
| | 0 | 0 | 34 | 0 | 0 | 0 | 0 | 0 | 0 | 0 | 0 | 0 | 0 | 99 |
| | 0 | 0 | 0 | 0 | 0 | 0 | 99 | 0 | 0 | 0 | 0 | 0 | 0 | 0 |
| | 0 | 0 | 27 | 0 | 0 | 99 | 0 | 0 | 0 | 0 | 0 | 0 | 99 | 0 |
| | 0 | 0 | 0 | 0 | 0 | 0 | 0 | 0 | 0 | 0 | 99 | 0 | 0 | 0 |
| | 0 | 0 | 0 | 0 | 0 | 0 | 0 | 0 | 99 | 0 | 0 | 0 | 0 | 0 |
| | 0 | 0 | 0 | 0 | 0 | 0 | 0 | 0 | 0 | 0 | 0 | 0 | 0 | 0 |
| | 66 | 0 | 0 | 0 | 40 | 0 | 0 | 0 | 0 | 0 | 0 | 0 | 0 | 0 |
| | 0 | 0 | 0 | 0 | 60 | 0 | 0 | 0 | 0 | 0 | 0 | 0 | 0 | 0 |
| | 0 | 99 | 39 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 |
| | 66 | 99 | 39 | 99 | 60 | 99 | 99 | 99 | 99 | 99 | 99 | 99 | 99 | 99 |

| 29 | 30 | 31 | 32 | 33 | 34 | 35 | 36 | 37 | 38 | 39 | 40 | 41 |
|---|---|---|---|---|---|---|---|---|---|---|---|---|
| 0 | 0 | 99 | 0 | 0 | 0 | 0 | 0 | 0 | 0 | 0 | 0 | 0 |
| 0 | 0 | 0 | 0 | 0 | 0 | 99 | 0 | 0 | 0 | 0 | 87 | 75 |
| 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 0 | 0 | 0 |
| 99 | 0 | 0 | 0 | 0 | 0 | 0 | 0 | 0 | 0 | 0 | 0 | 0 |
| 0 | 0 | 0 | 0 | 0 | 0 | 0 | 99 | 0 | 0 | 0 | 0 | 6 |
| 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 98 | 0 | 0 |
| 0 | 0 | 0 | 0 | 0 | 0 | 0 | 0 | 0 | 0 | 0 | 0 | 0 |
| 0 | 0 | 0 | 0 | 0 | 99 | 0 | 0 | 0 | 0 | 0 | 0 | 8 |
| 0 | 0 | 0 | 0 | 0 | 0 | 0 | 0 | 0 | 0 | 0 | 10 | 0 |
| 0 | 0 | 0 | 0 | 0 | 0 | 0 | 0 | 0 | 0 | 0 | 0 | 0 |
| 0 | 0 | 0 | 0 | 0 | 0 | 0 | 0 | 99 | 99 | 0 | 0 | 0 |
| 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 0 | 0 | 8 |
| 0 | 99 | 0 | 0 | 0 | 0 | 0 | 0 | 0 | 0 | 0 | 0 | 0 |
| 0 | 0 | 0 | 0 | 99 | 0 | 0 | 0 | 0 | 0 | 0 | 0 | 0 |
| 0 | 0 | 0 | 99 | 0 | 0 | 0 | 0 | 0 | 0 | 0 | 0 | 0 |
| 0 | 0 | 0 | 0 | 0 | 0 | 0 | 0 | 0 | 0 | 0 | 0 | 0 |
| 99 | 99 | 99 | 99 | 99 | 99 | 99 | 99 | 99 | 99 | 98 | 87 | 75 |

| | CDR3 | | | | | | | | | | | | | | |
|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 |
| P | 11 | 10 | 8 | 7 | 7 | 7 | 3 | 3 | 3 | 2 | 1 | 1 | 1 | 0 | 0 |
| A | 6 | 6 | 6 | 6 | 6 | 5 | 4 | 4 | 4 | 4 | 1 | 1 | 1 | 0 | 0 |
| G | 4 | 4 | 6 | 6 | 6 | 6 | 7 | 6 | 7 | 5 | 2 | 2 | 2 | 0 | 0 |
| M | 2 | 2 | 2 | 2 | 2 | 2 | 2 | 2 | 2 | 1 | 0 | 0 | 0 | 0 | 0 |
| I | 4 | 4 | 4 | 4 | 4 | 4 | 3 | 3 | 3 | 2 | 1 | 1 | 1 | 0 | 0 |
| L | 8 | 7 | 8 | 7 | 7 | 7 | 6 | 6 | 6 | 5 | 1 | 1 | 1 | 0 | 0 |
| V | 4 | 4 | 6 | 6 | 6 | 6 | 7 | 6 | 7 | 5 | 2 | 2 | 2 | 0 | 0 |
| F | 3 | 3 | 4 | 4 | 3 | 3 | 4 | 4 | 3 | 3 | 1 | 1 | 1 | 0 | 0 |
| W | 1 | 1 | 2 | 2 | 2 | 2 | 2 | 2 | 2 | 2 | 1 | 1 | 1 | 0 | 0 |
| C | 3 | 3 | 4 | 4 | 4 | 4 | 4 | 4 | 4 | 3 | 1 | 1 | 1 | 0 | 0 |
| S | 9 | 10 | 10 | 10 | 9 | 9 | 7 | 7 | 7 | 5 | 2 | 2 | 2 | 0 | 0 |
| T | 9 | 9 | 7 | 7 | 7 | 7 | 3 | 3 | 3 | 2 | 1 | 1 | 1 | 0 | 0 |
| N | 5 | 6 | 4 | 5 | 4 | 4 | 2 | 2 | 2 | 2 | 0 | 0 | 0 | 0 | 0 |
| Q | 2 | 2 | 2 | 2 | 2 | 2 | 1 | 1 | 1 | 1 | 0 | 0 | 0 | 0 | 0 |
| Y | 4 | 4 | 4 | 4 | 4 | 4 | 3 | 3 | 3 | 2 | 1 | 1 | 1 | 0 | 0 |
| H | 7 | 7 | 5 | 5 | 5 | 5 | 2 | 2 | 2 | 2 | 0 | 1 | 0 | 0 | 0 |
| R | 8 | 8 | 8 | 7 | 8 | 7 | 6 | 6 | 6 | 4 | 2 | 2 | 2 | 0 | 0 |
| K | 2 | 2 | 2 | 2 | 2 | 2 | 1 | 1 | 1 | 1 | 0 | 0 | 0 | 0 | 0 |
| D | 4 | 4 | 4 | 4 | 4 | 4 | 2 | 2 | 2 | 2 | 1 | 1 | 1 | 0 | 0 |
| E | 1 | 1 | 2 | 2 | 2 | 2 | 1 | 2 | 2 | 1 | 0 | 0 | 0 | 0 | 0 |
| | 1 | 2 | 3 | 5 | 7 | 9 | 30 | 31 | 32 | 45 | 82 | 82 | 83 | 100 | 100 |
| | 11 | 10 | 10 | 10 | 9 | 9 | 7 | 7 | 7 | 5 | 2 | 2 | 2 | 0 | 0 |
| | 16 | 17 | 18 | 19 | 20 | 21 | 22 | 23 | 24 | 25 | 26 | 27 | 28 | 29 | 30 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 | 0 | 0 |
| | 0 | 0 | | 0 | 0 | 0 | 0 | 0 | 0 | 0 | 0 | 0 | 0 | 0 | 0 |
| | 100 | 100 | 100 | 100 | 100 | 100 | 100 | 100 | 100 | 100 | 100 | 100 | 100 | 100 | 100 |
| | 0 | 0 | 0 | 0 | 0 | 0 | 0 | 0 | 0 | 0 | 0 | 0 | 0 | 0 | 0 |

| | Frame4 | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 13 |
| P | 0 | 0 | 0 | 0 | 0 | 0 | 0 | 0 | 0 | 0 | 0 | 0 | 0 |
| A | 0 | 0 | 0 | 0 | 0 | 0 | 0 | 0 | 0 | 0 | 0 | 0 | 0 |
| G | 13 | 0 | 0 | 99 | 0 | 99 | 0 | 0 | 0 | 0 | 0 | 0 | 0 |
| M | 0 | 0 | 0 | 0 | 0 | 0 | 0 | 0 | 0 | 0 | 0 | 0 | 0 |
| I | 0 | 0 | 0 | 0 | 0 | 0 | 0 | 0 | 0 | 0 | 0 | 0 | 0 |
| L | 0 | 0 | 0 | 0 | 0 | 0 | 0 | 0 | 0 | 0 | 0 | 0 | 0 |
| V | 0 | 0 | 0 | 0 | 0 | 0 | 0 | 0 | 99 | 0 | 99 | 0 | 0 |
| F | 0 | 0 | 0 | 0 | 0 | 0 | 0 | 0 | 0 | 0 | 0 | 0 | 0 |
| W | 0 | 0 | 99 | 0 | 0 | 0 | 0 | 0 | 0 | 0 | 0 | 0 | 0 |
| C | 0 | 0 | 0 | 0 | 0 | 0 | 0 | 0 | 0 | 0 | 0 | 0 | 0 |
| S | 0 | 7 | 0 | 0 | 0 | 0 | 0 | 0 | 0 | 0 | 0 | 99 | 99 |
| T | 0 | 0 | 0 | 0 | 0 | 0 | 100 | 0 | 0 | 99 | 0 | 0 | 0 |
| N | 13 | 0 | 0 | 0 | 0 | 0 | 0 | 0 | 0 | 0 | 0 | 0 | 0 |
| Q | 0 | 0 | 0 | 0 | 99 | 0 | 0 | 99 | 0 | 0 | 0 | 0 | 0 |
| Y | 0 | 91 | 0 | 0 | 0 | 0 | 0 | 0 | 0 | 0 | 0 | 0 | 0 |
| H | 0 | 0 | 0 | 0 | 0 | 0 | 0 | 0 | 0 | 0 | 0 | 0 | 0 |
| R | 0 | 0 | 0 | 0 | 0 | 0 | 0 | 0 | 0 | 0 | 0 | 0 | 0 |
| K | 0 | 0 | 0 | 0 | 0 | 0 | 0 | 0 | 0 | 0 | 0 | 0 | 0 |
| D | 64 | 0 | 0 | 0 | 0 | 0 | 0 | 0 | 0 | 0 | 0 | 0 | 0 |
| E | 7 | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 | 0 |
| | 64 | 9 | 99 | 99 | 99 | 99 | 100 | 99 | 99 | 99 | 99 | 99 | 99 |

TABLE 12
Primers and templates used for generation, selection and sequencing of VHH library.
Primer sequences used in this study and VHH frame template sequences. PCR cycling
conditions were also shown. (SEQ ID NOS: 15102-15157)
Primers and Templates for VHH library generation templates

| | |
|---|---|
| frames_v1 | GTAACCCACTCGTGCACCCAACTGATCTTCAGCATCTTTTACTTTCACCAGCGTTTCTGGGTGAGCAAAAACAGGAAGGCA |
| | AAATGCCGCAAAAAAGGGAATAAGGGCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATAAACAAATAGGGGTTCCGCGCACATT |
| | TCCCCGAAAAGTGCCACCTGACGTCGACGGATCGGGAGTTTAAACTAATACGACTCACTATAGGGTCTAGAAATAATTTTG |
| | TTTAACTTTAAGAAGGAGATATACCATGCAGGTTCAACTGGTTGAATCTGGTGGCGCATTAGTTCAACCGGGTGGTAGCCT |
| | TCGTCTGAGCTGCGCCGCAAGCGGACGTACATTCAGCAGCTATGCAATGGGTTGGTTTCGCCAGGCACCTGGTAAAGAAC |
| | GTGAATTTGTGGCAGCCATTTCAAGCAGTGGTGGCACCTACTATGCCGATTCTGTTAAAGGTCGCTTCACTATTTCACGCG |
| | ATGATGCGCGTAATACCGTGTACTTACAGATGAACTCACTGAAACCTGAGGATACCGCCGTTTATTATTGTGCGGCAGGCC |
| | GTGGAGGCGGTAGCGACTATTGGGGTCAGGGCACACAAGTCACGGTCTCAAGCGGCGGaagctctGGtGAACAGAAACTG |
| | ATTTCAGAAGAGGATCTGGCAGAACAGAAATTAATCTCAGAGGAAGACCTGGGTTCTGGTGAGCAAAAGTTAATTTCTGA |
| | AGAAGACCTGGCAGGTGGCTCTGGTGATGGAGGTAGCTCTGGCGATGGTGCGGATGGCAGCGGTGGTGCATCTGGTTCA |
| | AGCGCTTACAGCGATGATGTTAGTGCAAGCGGTCGTACCTACCGCATCTTCTCATATAACTACGCCAGCTATAGCGACTGG |
| | GTTTAAACCGGCTGCTAACAAAGCCCGAAAGGAAGCTGAGTTGGCTGCTGCCACCGCTGAGCAATAACTAGCATAACCCC |
| | TTGGGGCCTCTAAACGGGTCTTGAGGGGTTTTTTGCTGAAAGGAGGAACTATATCCGGTGAAAGGTTGGGCTTCGGAATC |
| | GTTTTCCGGGACGCCGGCTGGATGATCCTCCAGCGCGGGGATCTCATGCTGGAGTTCTTCGCCCACCCCAACTTGTTTATT |
| | GCAGCTTATAATGGTTACAAATAAAGCAATAGCATCACAAATTTCACAAATAAAGCATTTTTTTCACTGCATTCTAGTTGTG |
| | GTTTGTCCAAACTCATCAATGTATCTTATCATGTCTGTATACCGTCGACCTCTAGCTAGAGCTTGGC |
| frames_v2 | GTAACCCACTCGTGCACCCAACTGATCTTCAGCATCTTTTACTTTCACCAGCGTTTCTGGGTGAGCAAAAACAGGAAGGCA |
| | AAATGCCGCAAAAAAGGGAATAAGGGCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATAAACAAATAGGGGTTCCGCGCACATT |
| | TCCCCGAAAAGTGCCACCTGACGTCGACGGATCGGGAGTTTAAACTAATACGACTCACTATAGGGTCTAGAAATAATTTTG |
| | TTTAACTTTAAGAAGGAGATATACCATGGAAGTTCAACTGGTTGAATCTGGTGGCGGTTTAGTTCAAGCCGGTGATAGCCT |
| | TCGTCTGAGCTGCGCCGCAAGCGGACGTACATTCAGCAGCTATGCAATGGGTTGGTTTCGCCAGGCACCTGGTAAAGAAC |
| | GTGAATTTGTGGCAGCCATTTCAAGCAGTGGTGGCACCTACTATGCCGATTCTGTTCGTGGTCGCTTCAGCATTTCAGCAG |
| | ATTCTGCGAAGAATACCGTGTACTTACAGATGAACTCACTGAAACCTGAGGATACCGCCGTTTATTATTGTGCGGCAGGCC |
| | GTGGAGGCGGTAGCGACTATTGGGGTCAGGGCACACAAGTCACGGTCTCAAGCGGCGGaagctctGGtGAACAGAAACTG |
| | ATTTCAGAAGAGGATCTGGCAGAACAGAAATTAATCTCAGAGGAAGACCTGGGTTCTGGTGAGCAAAAGTTAATTTCTGA |
| | AGAAGACCTGGCAGGTGGCTCTGGTGATGGAGGTAGCTCTGGCGATGGTGCGGATGGCAGCGGTGGTGCATCTGGTTCA |
| | AGCGCTTACAGCGATGATGTTAGTGCAAGCGGTCGTACCTACCGCATCTTCTCATATAACTACGCCAGCTATAGCGACTGG |
| | GTTTAAACCGGCTGCTAACAAAGCCCGAAAGGAAGCTGAGTTGGCTGCTGCCACCGCTGAGCAATAACTAGCATAACCCC |
| | TTGGGGCCTCTAAACGGGTCTTGAGGGGTTTTTTGCTGAAAGGAGGAACTATATCCGGTGAAAGGTTGGGCTTCGGAATC |
| | GTTTTCCGGGACGCCGGCTGGATGATCCTCCAGCGCGGGGATCTCATGCTGGAGTTCTTCGCCCACCCCAACTTGTTTATT |
| | GCAGCTTATAATGGTTACAAATAAAGCAATAGCATCACAAATTTCACAAATAAAGCATTTTTTTCACTGCATTCTAGTTGTG |
| | GTTTGTCCAAACTCATCAATGTATCTTATCATGTCTGTATACCGTCGACCTCTAGCTAGAGCTTGGC |
| frames_v3 | GTAACCCACTCGTGCACCCAACTGATCTTCAGCATCTTTTACTTTCACCAGCGTTTCTGGGTGAGCAAAAACAGGAAGGCA |
| | AAATGCCGCAAAAAAGGGAATAAGGGCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATAAACAAATAGGGGTTCCGCGCACATT |
| | TCCCCGAAAAGTGCCACCTGACGTCGACGGATCGGGAGTTTAAACTAATACGACTCACTATAGGGTCTAGAAATAATTTTG |
| | TTTAACTTTAAGAAGGAGATATACCATGCAGGTTCAACTGCAAGAATCTGGTGGCGGTTTAGTTCAAGCCGGTGGTAGCCT |
| | TCGTCTGAGCTGCGCCGCAAGCGGACGTACATTCAGCAGCTATGCAATGGGTTGGTTTCGCCAGGCACCTGGTAAAGAAC |
| | GTGAATTTGTGGCAGCCATTTCAAGCAGTGGTGGCACCTACTATGCCGATTCTGTTAAAGGTCGCTTCACTATTTCACGCG |
| | ATAATGCGAAGAATACCGTGTACTTACAGATGAACTCACTGAAACCTGAGGATACCGCCGTTTATTATTGTGCGGCAGGCC |
| | GTGGAGGCGGTAGCGACTATTGGGGTCAGGGCACACAAGTCACGGTCTCAAGCGGCGGaagctctGGtGAACAGAAACTG |
| | ATTTCAGAAGAGGATCTGGCAGAACAGAAATTAATCTCAGAGGAAGACCTGGGTTCTGGTGAGCAAAAGTTAATTTCTGA |
| | AGAAGACCTGGCAGGTGGCTCTGGTGATGGAGGTAGCTCTGGCGATGGTGCGGATGGCAGCGGTGGTGCATCTGGTTCA |
| | AGCGCTTACAGCGATGATGTTAGTGCAAGCGGTCGTACCTACCGCATCTTCTCATATAACTACGCCAGCTATAGCGACTGG |
| | GTTTAAACCGGCTGCTAACAAAGCCCGAAAGGAAGCTGAGTTGGCTGCTGCCACCGCTGAGCAATAACTAGCATAACCCC |
| | TTGGGGCCTCTAAACGGGTCTTGAGGGGTTTTTTGCTGAAAGGAGGAACTATATCCGGTGAAAGGTTGGGCTTCGGAATC |
| | GTTTTCCGGGACGCCGGCTGGATGATCCTCCAGCGCGGGGATCTCATGCTGGAGTTCTTCGCCCACCCCAACTTGTTTATT |
| | GCAGCTTATAATGGTTACAAATAAAGCAATAGCATCACAAATTTCACAAATAAAGCATTTTTTTCACTGCATTCTAGTTGTG |
| | GTTTGTCCAAACTCATCAATGTATCTTATCATGTCTGTATACCGTCGACCTCTAGCTAGAGCTTGGC |

| stage 1 PCR1 primers | | |
|---|---|---|
| (CDR2 randomization) | cycling condition | |
| sense | GCGGCCGCTGGGCACCTTTTGGTGCCCAGCGGCCGCCACCTGACGT | 95° C. 1 mins, (95° C. 15s, 55° |
| | CCGAGGATCGGGAG | C. 20s) 65° C. 40s) X30, 65° C. |
| antisense | GBTAATGGHTGCCACAAATTCACGTTC | 2 mins |
| stage 1 PCR2 primers | | |
| (CDR2 randomization) | | |
| sense | NNBNNBNNBNNBNNBACCDACTATGCCGATTCTGTT | 95° C. 1 mins, (95° C. 15s, 55° |
| antisense | GCGGCCGCTCCGCAGCTTTTGCTGCGGAGCGGCCGCGCCAAGCTCT | C. 20s) 65° C. 40s) X30, 65° C. |
| | AGCTAGAGGTCGAC | 2 mins |
| stage 2 PCR1 primers | | |
| (CDR1 randomization) | cycling condition | |
| sense | GCGGCCGCTGGGCACCTTTTGGTGCCCAGCGGCCGCGTAACCCACT | 95° C. 1 mins, (95° C. 15s, 55° |
| | CGTGCACCCAACTG | C. 20s) 65° C. 40s) X30, 65° C. |
| antisense | mixture of the following 2 primers at 1:1 ratio | 2 mins |
| | TCCGCTTGCGGCGCAGCTCAGACGAAGG | |
| | TCCGCTTGCGGTGCAGCTCAGACGAAGG | |
| stage 2 PCR2 primers | | |
| (CDR1 randomization) | cycling condition | |
| sense | mixture of the following 2 primers at 1:1 ratio | 95° C. 1 mins, (95° C. 15s, 55° |
| | NNBNNBNNBNNBNNBNNBNNBATGGGTTGGTTTCGCCAGGCACCTG | C. 20s) 65° C. 40s) X30, 65° C. |
| | GT | 2 mins |
| | NNBNNBNNBNNBNNBNNBNNBATGGGTTGGTATCGCCAGGCACCTG | |
| | GT | |
| antisense | GCGGCCGCTCCGCAGCTTTTGCTGCGGAGCGGCCGCGCCAAGCTCT | |
| | AGCTAGAGGTCGAC | |
| stage 3 PCR1 primers | | |
| (CDR3 randomization) | cycling condition | |
| sense | GCGGCCGCTGGGCACCTTTTGGTGCCCAGCGGCCGCCACCTGACGT | 95° C. 1 mins, (95° C. 15s, 55° |
| | CGACGGATCGGGAG | C. 20s) 65° C. 40s) X30, 65° C. |
| antisense | mixture of the following 10 primers at | 2 mins |
| | 66:8:7:6:13:66:8:7:6:13 ratio | |
| | VNNVNNTGCCGCACAATAATAAACGGCGGTATCCTCAGGTTTCAG | |
| | VNNVNNACGCGCACAATAATAAACGGCGGTATCCTCAGGTTTCAG | |
| | VNNVNNTGTCGCACAATAATAAACGGCGGTATCCTCAGGTTTCAG | |
| | VNNVNNAACCGCACAATAATAAACGGCGGTATCCTCAGGTTTCAG | |
| | VNNVNNTGCATTACAATAATAAACGGCGGTATCCTCAGGTTTCAG | |
| | VNNVNNVNNVNNVNNVNNTGCCGCACAATAATAAACGGCGGTATC | |
| | VNNVNNVNNVNNVNNVNNACGCGCACAATAATAAACGGCGGTATC | |
| | VNNVNNVNNVNNVNNVNNTGTCGCACAATAATAAACGGCGGTATC | |
| | VNNVNNVNNVNNVNNVNNAACCGCACAATAATAAACGGCGGTATC | |
| | VNNVNNVNNVNNVNNVNNTGCATTACAATAATAAACGGCGGTATC | |
| stage 3 PCR2 primers | | |
| (CDR3 randomization) | cycling condition | |
| sense | mixture of the following 10 primers at | 95° C. 1 mins, (95° C. 15s, 55° |
| | 57:14:13:8:8:57:14:13:8:8 ratio | C. 20s) 65° C. 40s) X30, 65° C. |
| | NNBNNBNNBNNBGACTATTGGGGTCAGGGCACACAAGTCACGGTC | 2 mins |
| | NNBNNBNNBNNBAACTATTGGGGTCAGGGCACACAAGTCACGGTC | |
| | NNBNNBNNBNNBGGCTATTGGGGTCAGGGCACACAAGTCACGGTC | |
| | NNBNNBNNBNNBGAGTATTGGGGTCAGGGCACACAAGTCACGGTC | |
| | NNBNNBNNBNNBGACTCTTGGGGTCAGGGCACACAAGTCACGGTC | |
| | NNBNNBNNBNNBNNBNNBNNBGACTATTGGGGTCAGGGCACACAA | |
| | NNBNNBNNBNNBNNBNNBNNBAACTATTGGGGTCAGGGCACACAA | |
| | NNBNNBNNBNNBNNBNNBNNBGGCTATTGGGGTCAGGGCACACAA | |
| | NNBNNBNNBNNBNNBNNBNNBGAGTATTGGGGTCAGGGCACACAA | |
| | NNBNNBNNBNNBNNBNNBNNBGACTCTTGGGGTCAGGGCACACAA | |
| antisense | GCGGCCGCTCCGCAGCTTTTGCTGCGGAGCGGCCGCGCCAAGCTCT | |
| | AGCTAGAGGTCGAC | |

Primers for ribosome display and in vitro selection

| DNA | | |
|---|---|---|
| recovery primer pair | cycling condition | |
| sense | AAACTAATACGACTCACTATAGGGTCTAGAAATAATTTTGTTTAAC | 95° C. 1 mins, (95° C. 15s, 65° |
| | TTTAAGAAGGAG | C.1 5s, 72° C. 20s) XN, 72° C. |
| antisense | ATAGCTGGCGTAGTTATATGAGAAGATG | 10s |

reverse transcription primer
CCAGTCGCTATAGCTGGCGTAGTTA
TATGAG

Primers for sequencing library generation

| Primers for first PCR | cycling condition | |
|---|---|---|
| sense | mixture of the following 8 primers at equal | 95° C. 1 mins, (95° C. 15s, 60° |
| | molar ratio | C. 15s, 72° C. 20s) X4, 72° C. |
| | ACACGACGCTCTTCCGATCTNNCTTTAAGAAGGAGATATACCATG | 1 mins |
| | ACACGACGCTCTTCCGATCTNNNNCTTTAAGAAGGAGATATACCATG | |
| | ACACGACGCTCTTCCGATCTNNNNNNCTTTAAGAAGGAGATATACC | |
| | ATG | |
| | ACACGACGCTCTTCCGATCTNNNNNNNNCTTTAAGAAGGAGATATA | |
| | CCATG | |
| | ACACGACGCTCTTCCGATCTNNNNNNNNNNCTTTAAGAAGGAGATA | |
| | TACCATG | |
| | ACACGACGCTCTTCCGATCTNNNNNNNNNNNCTTTAAGAAGGAGA | |
| | TATACCATG | |
| | ACACGACGCTCTTCCGATCTNNNNNNNNNNNNCTTTAAGAAGGAG | |
| | ATATACCATG | |
| | ACACGACGCTCTTCCGATCTNNNNNNNNNNNNNCTTTAAGAAGGA | |
| | GATATACCATG | |
| antisense | mixture of the following 8 primers at equal | |
| | molar ratio, or use subsets for multiplexing | |
| | samples, underlined sequences are indexes | |
| | CAGACGTGTGCTCTTCCGATCTGCTTCTGAAATCAGTTTCTGTTC | |
| | CAGACGTGTGCTCTTCCGATCTAACTTCTGAAATCAGTTTCTGTTC | |
| | CAGACGTGTGCTCTTCCGATCTTCGCTTCTGAAATCAGTTTCTGTTC | |
| | CAGACGTGTGCTCTTCCGATCTCGTACTTCTGAAATCAGTTTCTGTT | |
| | C | |
| | CAGACGTGTGCTCTTCCGATCTCTTGGCTTCTGAAATCAGTTTCTGT | |
| | TC | |
| | CAGACGTGTGCTCTTCCGATCTACCAACGCTTCTGAAATCAGTTTCT | |
| | GTTC | |
| | CAGACGTGTGCTCTTCCGATCTGGATCTAGCTTCTGAAATCAGTTTC | |
| | TGTTC | |
| | CAGACGTGTGCTCTTCCGATCTTAGCTGCTCCTTCTGAAATCAGTTT | |
| | CTGTTC | |
| Primers | | |
| for second PCR | condition cycling | |
| sense | AATGATACGGCGACCACCGAGATCTACACTCTTTCCCTACACGACGC | 95° C. 1 mins, (95° C. 15s, 65° |
| | TCTTCCGATCT | C. 15s, 72° C. 20s) XN, 72° C. |
| antisense | CAAGCAGAAGACGGCATACGAGATGTGACTGGAGTTCAGACGTGTG | 20s |
| | CTCTTCCGATCT | |

TABLE 13
Affinity maturation subtracted amino acid profile for SR4 and SR6. Position-wise post- minus pre- affinity maturation
amino acid profile for SR4 and SR6. Numbers are percent point change of each amino acid after affinity maturation.
SR4 post- minus pre- affinity maturation a.a. profile
Frame1

| | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| P | −0.01 | 0.00 | −1.78 | −0.94 | −0.05 | 0.00 | −0.70 | 0.00 | 0.00 | 0.00 | −0.59 | 0.00 | −0.12 | −0.02 |
| A | −0.01 | −0.05 | 0.00 | 0.00 | −0.86 | 1.35 | −0.01 | −0.05 | 0.10 | 0.15 | 0.00 | −0.92 | 0.00 | 0.45 |
| G | −1.76 | −0.06 | 0.00 | 0.00 | −1.83 | 1.73 | 0.00 | 0.83 | −4.74 | −2.74 | 0.00 | −0.72 | −0.01 | −0.40 |
| M | 0.01 | 0.00 | 0.00 | −0.11 | 0.00 | 0.00 | −0.01 | −0.01 | 0.00 | 0.00 | 0.10 | 0.00 | 0.00 | 0.00 |
| I | 0.00 | 0.00 | 0.00 | −0.05 | 0.33 | 0.04 | −1.18 | −0.01 | 0.01 | 0.01 | 0.00 | 0.00 | 0.00 | −0.01 |
| L | 0.00 | 0.00 | 0.02 | 4.17 | −0.02 | 0.05 | 0.69 | 0.00 | 0.01 | 0.01 | 2.59 | 0.04 | 0.64 | 0.00 |
| V | 0.02 | 1.96 | 0.01 | −0.05 | 3.27 | 12.60 | −0.03 | 0.10 | 1.83 | 1.59 | −0.01 | 2.02 | 0.00 | 0.15 |
| F | 0.00 | 0.00 | 0.00 | 0.00 | 0.46 | 0.00 | 0.04 | 0.00 | 0.00 | 0.00 | 0.00 | 0.13 | 0.00 | 0.00 |
| W | −0.03 | −0.01 | 0.00 | 0.00 | −0.02 | −0.06 | 0.00 | −0.01 | 0.01 | −0.05 | 0.00 | 0.00 | 0.00 | 0.00 |
| C | 0.00 | −0.01 | 0.00 | 0.00 | −0.01 | 0.00 | 0.00 | 0.19 | 0.01 | 0.02 | 0.00 | 0.00 | 0.00 | 0.00 |
| S | 0.00 | −1.77 | −0.06 | −0.03 | −0.01 | −0.02 | 0.34 | −0.05 | 0.01 | 0.01 | −0.04 | 0.00 | −0.57 | −0.07 |
| T | 0.00 | 0.00 | 0.00 | −1.79 | −0.02 | 0.03 | 0.89 | 0.00 | 0.00 | 0.00 | −0.80 | −0.02 | −0.01 | 0.02 |
| N | 0.02 | 0.00 | 0.00 | −0.03 | −0.02 | 0.00 | −0.03 | 0.00 | −0.04 | 0.00 | 0.00 | −0.05 | 0.00 | 0.00 |
| Q | 0.02 | 0.00 | 1.83 | −1.10 | 0.00 | 0.08 | 0.00 | −0.02 | 0.00 | −0.01 | −1.12 | 0.00 | 0.65 | 0.01 |
| Y | 0.03 | 0.00 | 0.00 | 0.00 | 0.00 | 0.03 | 0.02 | 0.01 | 0.00 | 0.00 | 0.00 | 0.00 | 0.00 | 0.00 |
| H | −0.01 | 0.00 | 0.02 | 0.00 | 0.00 | 0.00 | 0.00 | 0.00 | 0.00 | 0.00 | 0.00 | 0.00 | 0.12 | 0.00 |
| R | 0.00 | 0.00 | 0.02 | −0.07 | 0.00 | −1.43 | −0.01 | −0.78 | 2.96 | 1.10 | −0.12 | 0.00 | −0.70 | 0.00 |
| K | 1.20 | −0.03 | −0.02 | 0.00 | 0.00 | 2.26 | −0.04 | −0.01 | 0.01 | 0.00 | 0.00 | 0.00 | 0.03 | 0.00 |
| D | 0.59 | −0.02 | −0.02 | 0.00 | −1.21 | −0.26 | 0.00 | −0.17 | 0.00 | 0.00 | 0.00 | −0.47 | 0.00 | −0.01 |
| E | −0.01 | 0.01 | 0.00 | 0.00 | −0.01 | #### | 0.02 | −0.02 | −0.18 | −0.08 | 0.00 | −0.01 | −0.02 | −0.12 |

SR4 post- minus pre- affinity maturation a.a. profile
Frame1

| | 15 | 16 | 17 | 18 | 19 | 20 | 21 | 22 | 23 | 24 | 25 | 26 |
|---|---|---|---|---|---|---|---|---|---|---|---|---|
| P | 0.00 | 0.00 | −0.97 | −0.55 | −0.05 | 0.00 | 0.00 | 0.00 | −0.06 | −0.02 | 0.11 | 0.00 |
| A | −0.04 | −0.01 | 0.00 | 0.00 | 0.00 | 0.00 | 0.00 | 0.00 | −0.40 | 0.31 | 0.05 | 0.01 |
| G | 0.20 | 0.11 | −0.01 | 0.00 | −0.04 | 0.00 | −0.37 | −0.15 | −0.02 | −0.01 | 0.00 | 0.09 |
| M | 0.00 | 0.00 | 0.00 | 0.00 | 0.00 | −0.01 | 0.00 | 0.00 | 0.00 | 0.00 | 0.00 | 0.00 |
| I | 0.00 | −0.01 | −0.01 | 0.08 | 0.00 | −0.42 | 0.09 | 0.00 | 0.00 | 0.00 | 0.00 | 0.00 |
| L | 0.00 | 0.00 | −0.07 | 1.27 | 0.00 | 2.06 | −0.02 | 0.00 | 0.00 | 0.00 | 0.16 | 0.00 |
| V | −0.06 | 0.15 | −0.01 | −0.03 | 0.00 | −0.13 | 0.00 | 0.00 | 0.20 | 0.00 | 0.00 | 0.15 |
| F | 0.00 | 0.01 | 0.00 | −0.15 | 0.00 | −0.83 | 0.00 | −0.07 | 0.00 | 0.00 | 0.00 | 0.00 |
| W | 0.00 | 0.00 | 0.00 | 0.00 | 0.00 | 0.00 | 0.00 | −0.07 | 0.00 | 0.00 | 0.00 | 0.00 |
| C | 0.05 | 0.00 | −0.01 | 0.00 | −0.03 | 0.00 | 0.18 | 2.05 | 0.00 | 0.00 | 0.00 | −0.11 |
| S | −0.03 | 0.00 | 1.63 | 0.00 | −0.11 | −0.61 | 0.37 | −0.68 | 0.14 | −0.05 | −0.90 | −0.17 |
| T | 0.00 | 0.00 | −0.50 | 0.00 | 0.00 | −0.01 | 0.01 | 0.00 | 0.20 | −0.17 | 0.56 | 0.00 |
| N | 0.00 | −0.07 | −0.01 | 0.00 | 0.00 | 0.00 | −0.06 | 0.00 | 0.00 | 0.00 | 0.00 | 0.00 |
| Q | 0.00 | 0.00 | 0.00 | −0.04 | 0.00 | 0.00 | 0.00 | 0.00 | 0.00 | 0.00 | 0.00 | 0.00 |
| Y | 0.00 | −0.06 | 0.00 | 0.00 | 0.00 | −0.03 | 0.00 | −0.21 | 0.00 | 0.00 | 0.01 | 0.00 |
| H | 0.00 | 0.00 | 0.00 | −0.56 | −0.29 | 0.00 | 0.00 | 0.00 | 0.00 | 0.00 | 0.00 | 0.00 |
| R | −0.01 | 0.00 | 0.00 | −0.03 | 0.52 | 0.00 | −0.22 | −0.86 | 0.00 | 0.00 | 0.00 | −0.01 |
| K | 0.00 | −0.02 | −0.03 | 0.00 | 0.00 | −0.01 | 0.00 | 0.00 | 0.00 | 0.00 | 0.00 | 0.00 |
| D | −0.12 | 0.01 | 0.00 | 0.00 | 0.00 | 0.00 | 0.00 | 0.00 | 0.00 | 0.00 | 0.00 | 0.04 |
| E | 0.00 | −0.12 | 0.00 | 0.00 | 0.00 | 0.00 | 0.00 | 0.00 | −0.06 | −0.07 | 0.00 | 0.00 |

CDR1

| | 1 | 2 | 3 | 4 | 5 | 6 | 7 | 8 | 9 | 10 |
|---|---|---|---|---|---|---|---|---|---|---|
| P | 0.00 | 0.20 | −0.51 | 0.00 | 0.00 | 0.00 | 0.00 | 0.00 | 0.00 | 0.00 |
| A | 0.00 | 0.00 | 0.00 | −0.01 | 0.00 | 0.00 | 0.00 | 0.00 | 0.00 | 0.00 |
| G | 0.00 | 0.00 | 0.00 | −0.61 | −0.15 | 0.00 | −0.20 | 0.00 | 0.00 | 0.00 |
| M | 0.00 | 0.00 | 0.06 | 0.00 | 0.00 | 0.00 | 0.00 | 0.00 | 0.00 | 0.00 |
| I | 0.06 | 0.00 | 0.00 | −0.09 | 0.00 | −0.01 | −0.02 | 0.00 | 0.00 | 0.00 |
| L | −0.16 | −0.37 | 1.29 | 0.00 | −0.01 | −0.01 | 0.00 | 0.00 | 0.00 | 0.00 |
| V | −0.03 | 0.00 | 0.00 | 0.00 | 0.00 | 0.00 | 0.00 | 0.00 | 0.00 | 0.00 |
| F | 0.79 | 0.00 | 0.00 | 0.00 | 0.03 | 0.01 | −0.13 | 0.00 | 0.00 | 0.00 |
| W | 0.00 | 0.00 | 0.00 | 0.00 | 0.02 | 0.00 | −0.04 | 0.00 | 0.00 | 0.00 |
| C | −0.02 | 0.00 | 0.00 | −0.79 | 1.48 | −0.34 | 3.59 | 0.00 | 0.00 | 0.00 |
| S | −0.21 | 0.00 | 0.00 | 1.80 | −0.81 | −0.14 | −1.65 | 0.00 | 0.00 | 0.00 |
| T | 0.00 | 0.00 | −0.01 | 0.00 | 0.00 | −0.01 | 0.00 | 0.00 | 0.00 | 0.00 |
| N | 0.00 | 0.00 | 0.00 | −0.15 | 0.01 | −0.58 | 0.00 | 0.00 | 0.00 | 0.00 |
| Q | 0.00 | 0.92 | −0.54 | 0.00 | 0.00 | 0.00 | 0.00 | 0.00 | 0.00 | 0.00 |
| Y | −0.42 | 0.00 | 0.00 | 0.00 | 0.18 | 1.90 | −0.28 | 0.00 | 0.00 | 0.00 |
| H | −0.01 | −0.04 | 0.00 | 0.00 | 0.00 | −0.71 | 0.00 | 0.00 | 0.00 | 0.00 |
| R | 0.00 | −0.59 | −0.31 | −0.15 | −0.76 | 0.00 | −1.26 | 0.00 | 0.00 | 0.00 |
| K | 0.00 | −0.15 | 0.00 | 0.00 | 0.00 | 0.00 | 0.00 | 0.00 | 0.00 | 0.00 |
| D | 0.00 | 0.00 | 0.00 | −0.01 | 0.00 | −0.13 | 0.00 | 0.00 | 0.00 | 0.00 |
| E | 0.00 | 0.02 | 0.00 | 0.00 | 0.00 | 0.00 | 0.00 | 0.00 | 0.00 | 0.00 |

Frame2

| | 1 | 2 | 3 | 4 | 5 | 6 | 7 | 8 | 9 | 10 | 11 | 12 | 13 | 14 | 15 | 16 | 17 | 18 | 19 |
|---|---|---|---|---|---|---|---|---|---|---|---|---|---|---|---|---|---|---|---|
| P | 0.00 | 0.00 | 0.00 | −0.01 | −0.05 | −0.36 | −0.01 | 0.46 | 0.00 | 0.00 | 0.00 | 0.09 | 0.00 | 0.00 | −0.01 | −0.03 | 0.00 | −0.01 | −0.13 |
| A | 0.02 | 0.01 | 0.00 | −0.03 | 0.00 | 0.00 | 0.17 | 0.01 | −0.04 | 0.00 | 0.01 | 0.00 | −0.03 | 0.00 | −0.93 | 0.81 | 0.00 | 0.01 | −0.66 |
| G | 0.00 | 0.61 | −0.28 | 0.00 | −0.04 | 0.00 | −0.04 | 0.00 | 0.55 | −0.01 | −0.64 | −0.01 | −0.87 | 0.00 | −0.15 | −0.05 | −0.89 | 0.00 | 0.00 |
| M | −3.71 | 0.00 | −0.01 | 0.00 | 0.00 | 0.00 | 0.00 | 0.00 | 0.00 | 0.00 | −0.01 | 0.00 | 0.00 | 0.00 | 0.00 | 0.00 | 0.00 | −0.12 | −0.51 |
| I | 0.61 | 0.00 | 0.00 | −0.53 | 0.00 | 0.00 | 0.00 | 0.00 | 0.00 | −0.44 | 0.00 | 0.00 | 0.00 | −0.65 | −0.24 | 0.00 | 0.07 | 2.47 | 0.00 |
| L | 1.09 | 0.00 | −0.13 | −2.29 | −0.15 | −0.45 | 0.00 | −0.05 | 0.00 | 0.00 | 0.00 | 0.44 | 0.00 | −1.57 | −0.06 | 0.00 | 0.00 | −0.17 | 0.00 |
| V | 3.31 | −0.06 | 0.00 | −0.28 | 0.00 | 0.00 | −0.06 | 0.00 | −0.16 | 0.00 | −0.46 | 0.00 | −0.81 | −0.16 | 2.27 | −0.20 | 0.00 | −0.16 | 0.00 |
| F | −0.01 | 0.00 | 0.00 | 4.90 | 0.00 | 0.00 | 0.00 | 0.00 | 0.00 | 0.00 | 0.00 | 0.00 | 0.00 | 4.49 | 0.00 | 0.00 | 0.00 | −0.89 | 0.00 |
| W | 0.00 | 0.00 | 2.38 | 0.00 | 0.00 | 0.00 | 0.00 | 0.00 | 0.00 | 0.00 | 0.00 | 0.41 | 0.00 | 0.00 | 0.00 | 0.00 | 0.00 | 0.00 | 0.00 |
| C | 0.00 | −0.08 | 0.30 | −0.11 | −0.31 | 0.00 | 0.00 | 0.00 | −0.05 | 0.00 | 0.00 | 0.01 | 0.00 | −0.10 | 0.00 | 0.00 | 0.20 | 0.00 | 0.00 |
| S | −0.01 | −0.10 | −0.03 | −1.00 | −0.21 | 0.00 | −0.07 | −0.14 | −0.28 | −0.01 | 0.00 | 0.00 | 0.00 | −0.95 | 0.00 | −0.18 | 1.07 | −0.16 | −0.56 |
| T | −0.09 | 0.00 | 0.00 | −0.02 | 0.00 | 0.00 | −0.01 | −0.03 | 0.00 | 0.00 | 0.00 | 0.00 | 0.00 | −0.01 | 0.00 | −0.21 | 0.00 | −0.46 | 2.06 |
| N | 0.00 | 0.00 | 0.00 | 0.00 | 0.00 | 0.00 | 0.00 | 0.00 | 0.00 | −0.17 | 0.00 | 0.00 | 0.00 | 0.00 | 0.00 | 0.00 | −0.16 | −0.49 | 0.00 |
| Q | −0.01 | 0.00 | 0.00 | 0.00 | 0.00 | 1.90 | 0.00 | 0.01 | 0.00 | −0.02 | −0.02 | 0.18 | 0.00 | 0.00 | 0.00 | 0.00 | 0.00 | 0.00 | 0.00 |
| Y | 0.00 | 0.00 | 0.00 | −0.61 | 0.00 | −0.01 | 0.00 | 0.00 | 0.00 | 0.01 | 0.00 | 0.00 | 0.00 | −1.05 | 0.00 | 0.00 | −0.01 | −0.01 | 0.00 |
| H | 0.00 | 0.00 | 0.00 | −0.01 | −0.49 | −0.20 | 0.00 | −0.24 | 0.00 | 0.00 | 0.00 | −0.01 | 0.00 | 0.00 | 0.00 | 0.00 | 0.00 | 0.00 | 0.00 |
| R | −0.14 | −0.02 | −2.23 | 0.00 | 1.24 | −0.84 | 0.00 | −0.02 | −0.03 | 0.13 | 0.00 | −1.11 | 0.00 | 0.00 | 0.00 | 0.00 | −0.28 | 0.00 | −0.01 |
| K | −1.07 | 0.00 | 0.00 | 0.00 | 0.00 | −0.02 | 0.00 | 0.00 | 0.00 | 0.52 | −0.12 | 0.00 | −0.17 | 0.00 | 0.00 | 0.00 | 0.00 | 0.00 | −0.19 |
| D | 0.00 | −0.37 | 0.00 | −0.01 | 0.00 | 0.00 | 0.00 | 0.00 | 0.01 | 0.00 | −0.16 | 0.00 | 0.00 | −0.01 | 0.00 | 0.00 | −0.01 | 0.00 | 0.00 |
| E | 0.00 | 0.00 | 0.00 | 0.00 | 0.00 | −0.01 | 0.01 | 0.00 | 0.00 | −0.01 | 1.40 | 0.00 | 1.88 | 0.00 | −0.89 | −0.13 | 0.00 | 0.00 | 0.00 |

CDR2

| | 1 | 2 | 3 | 4 | 5 | 6 | 7 | 8 | 9 | 10 |
|---|---|---|---|---|---|---|---|---|---|---|
| P | −0.16 | −0.35 | −0.09 | 0.00 | −0.03 | 0.00 | 0.00 | 0.00 | 0.00 | 0.00 |
| A | 0.00 | 0.00 | 0.97 | 0.00 | 0.00 | 0.00 | 0.00 | 0.00 | 0.00 | 0.00 |
| G | 0.00 | 0.00 | 0.02 | 0.00 | −0.03 | 0.00 | 0.00 | 0.00 | 0.00 | 0.00 |
| M | 0.00 | 0.00 | 0.00 | 0.00 | 0.00 | 0.00 | 0.00 | 0.00 | 0.00 | 0.00 |
| I | 0.00 | 0.00 | 0.00 | −0.35 | 0.00 | 0.00 | 0.00 | 0.00 | 0.00 | 0.00 |
| L | −1.45 | −1.07 | 0.00 | −1.32 | −0.19 | 0.00 | 0.00 | 0.00 | 0.00 | 0.00 |
| V | 0.00 | 0.00 | −0.28 | −0.17 | 0.00 | 0.00 | 0.00 | 0.00 | 0.00 | 0.00 |
| F | 0.00 | 0.00 | 0.00 | 3.03 | 0.00 | 0.00 | 0.00 | 0.00 | 0.00 | 0.00 |
| W | 0.00 | 0.00 | 0.00 | 0.00 | 0.00 | 0.00 | 0.00 | 0.00 | 0.00 | 0.00 |
| C | 0.00 | −0.01 | 0.00 | −0.10 | −0.70 | 0.00 | 0.00 | 0.00 | 0.00 | 0.00 |
| S | 0.00 | 0.00 | 0.02 | −0.85 | −0.27 | 0.00 | 0.00 | 0.00 | 0.00 | 0.00 |
| T | 0.00 | 0.00 | −0.48 | 0.00 | 0.00 | 0.00 | 0.00 | 0.00 | 0.00 | 0.00 |
| N | 0.00 | 0.11 | 0.00 | 0.00 | 0.00 | 0.00 | 0.00 | 0.00 | 0.00 | 0.00 |
| Q | 3.92 | −0.25 | 0.00 | 0.00 | 0.00 | 0.00 | 0.00 | 0.00 | 0.00 | 0.00 |
| Y | 0.00 | 0.06 | 0.00 | −0.24 | 0.00 | 0.00 | 0.00 | 0.00 | 0.00 | 0.00 |
| H | −1.01 | 2.68 | 0.00 | 0.00 | −0.61 | 0.00 | 0.00 | 0.00 | 0.00 | 0.00 |
| R | −1.18 | −1.14 | 0.00 | 0.00 | 1.83 | 0.00 | 0.00 | 0.00 | 0.00 | 0.00 |
| K | −0.09 | 0.00 | 0.00 | 0.00 | 0.00 | 0.00 | 0.00 | 0.00 | 0.00 | 0.00 |
| D | 0.00 | −0.02 | −0.01 | 0.00 | 0.00 | 0.00 | 0.00 | 0.00 | 0.00 | 0.00 |
| E | −0.02 | 0.00 | −0.15 | 0.00 | 0.00 | 0.00 | 0.00 | 0.00 | 0.00 | 0.00 |

Frame3

| | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| P | −0.15 | 0.00 | 0.00 | −0.08 | 0.00 | 0.00 | 0.00 | 0.00 | 0.00 | −0.06 | −0.01 | −0.05 | 0.00 | 0.00 |
| A | −0.09 | 0.00 | 0.00 | 0.99 | −0.04 | 0.00 | −0.34 | −0.01 | 0.01 | 0.00 | 0.00 | −0.18 | 0.00 | 0.00 |
| G | 0.00 | 0.00 | 0.00 | −0.02 | −0.55 | −0.07 | −0.18 | 0.00 | 0.31 | −0.08 | 0.00 | 0.00 | 0.00 | −0.36 |
| M | 0.00 | 0.00 | 0.00 | 0.00 | 0.00 | 0.00 | 0.00 | −0.17 | 0.00 | 0.00 | 0.00 | 0.00 | −0.34 | 0.00 |
| I | 0.17 | −0.01 | 0.00 | 0.00 | 0.00 | −0.04 | −0.18 | 0.00 | 0.00 | 0.00 | −0.38 | −0.01 | 1.25 | 0.07 |
| L | 0.00 | 0.00 | −0.02 | 0.00 | 0.00 | 0.00 | 0.00 | 0.00 | 0.00 | −0.09 | −2.36 | 0.00 | 0.23 | 0.00 |
| V | −0.01 | 0.00 | 0.00 | −0.25 | −0.73 | 0.00 | 1.48 | 0.00 | −0.08 | 0.00 | −0.10 | 0.00 | −0.07 | 0.00 |
| F | −0.01 | −0.50 | −0.35 | 0.00 | 0.00 | 0.00 | −0.01 | 0.00 | 0.00 | 0.00 | 4.08 | 0.00 | 0.00 | 0.00 |
| W | 0.00 | −0.01 | 0.00 | 0.00 | 0.00 | 0.00 | 0.00 | 0.00 | 0.00 | 0.00 | 0.00 | 0.00 | 0.00 | 0.00 |
| C | 0.00 | −0.37 | −0.43 | 0.00 | 0.00 | −0.17 | 0.00 | 0.00 | −0.16 | −0.57 | −0.01 | 0.00 | 0.00 | −0.05 |
| S | 0.29 | −0.02 | −0.07 | −0.04 | 0.00 | 0.55 | 0.00 | 0.01 | −0.10 | −0.21 | −0.67 | −0.25 | −0.01 | 1.41 |
| T | −0.17 | 0.00 | 0.00 | −0.53 | 0.00 | −0.02 | −0.01 | −0.11 | 0.00 | 0.00 | 0.00 | 0.49 | −0.38 | −0.01 |
| N | −0.06 | −0.77 | −0.68 | 0.00 | −0.20 | 0.04 | 0.00 | −0.16 | 0.00 | 0.00 | −0.05 | −0.01 | −0.01 | −0.02 |
| Q | 0.00 | 0.00 | 0.00 | 0.00 | 0.00 | 0.00 | 0.00 | −0.07 | 0.00 | 0.00 | 0.00 | 0.00 | 0.00 | 0.00 |
| Y | 0.00 | 2.48 | 2.13 | −0.01 | −0.27 | 0.00 | 0.00 | 0.00 | 0.00 | 0.00 | −0.48 | −0.01 | 0.00 | 0.00 |
| H | 0.00 | −0.70 | −0.46 | 0.00 | −0.02 | 0.00 | 0.00 | 0.00 | 0.00 | −0.41 | −0.02 | 0.00 | 0.00 | 0.00 |
| R | 0.00 | 0.00 | −0.02 | 0.00 | 0.00 | −0.30 | 0.00 | −0.26 | −0.07 | 1.42 | −0.01 | 0.00 | −0.10 | −1.06 |
| K | 0.00 | 0.00 | 0.00 | 0.00 | 0.00 | 0.00 | 0.00 | 1.27 | 0.00 | 0.00 | 0.00 | 0.00 | −0.56 | 0.00 |
| D | 0.00 | −0.09 | −0.10 | −0.06 | 1.71 | 0.00 | −0.77 | 0.00 | 0.10 | 0.00 | 0.00 | 0.00 | 0.00 | 0.00 |
| E | 0.00 | 0.00 | 0.00 | 0.00 | 0.09 | 0.00 | 0.00 | −0.50 | 0.00 | 0.00 | 0.00 | 0.00 | 0.00 | 0.00 |

Frame3

| | 15 | 16 | 17 | 18 | 19 | 20 | 21 | 22 | 23 | 24 | 25 | 26 | 27 | 28 |
|---|---|---|---|---|---|---|---|---|---|---|---|---|---|---|
| P | −0.07 | 0.00 | 0.00 | −0.03 | −0.03 | 0.00 | −0.19 | 0.00 | 0.00 | −1.03 | −0.39 | 0.00 | 0.00 | 0.00 |
| A | 0.00 | −0.16 | −0.11 | 0.07 | 0.00 | 0.00 | −0.41 | −0.35 | 0.00 | 0.00 | 0.00 | 0.01 | 0.00 | −0.01 |
| G | −0.04 | −0.94 | −0.65 | −0.05 | 0.00 | 0.00 | 0.00 | −2.11 | 0.00 | 0.00 | 0.00 | 0.00 | −0.01 | −0.15 |
| M | 0.00 | 0.00 | 0.00 | 0.00 | 0.00 | 0.00 | 0.00 | 0.00 | 0.00 | 0.00 | 0.00 | 2.73 | 0.00 | 0.00 |
| I | 0.00 | 0.00 | 0.00 | 0.00 | 0.00 | −0.65 | −0.08 | −0.11 | 0.00 | −0.12 | 0.00 | −0.05 | −0.05 | 0.00 |
| L | −0.24 | 0.00 | 0.00 | 0.00 | 0.26 | 0.00 | 0.00 | 0.01 | −0.02 | 2.46 | −0.21 | −0.23 | 0.00 | 0.00 |
| V | 0.00 | −0.87 | −1.02 | 0.01 | 0.00 | −0.01 | 0.00 | 3.21 | 0.00 | −0.01 | 0.00 | −0.63 | 0.00 | 0.00 |
| F | 0.00 | 0.00 | 0.00 | 0.00 | 0.00 | 0.00 | −0.01 | 0.01 | −0.19 | −0.29 | 0.00 | 0.00 | 0.00 | 0.00 |
| W | 0.00 | 0.00 | 0.00 | 0.00 | −0.15 | 0.00 | 0.00 | 0.00 | 0.00 | 0.00 | −0.01 | 0.00 | 0.00 | 0.00 |
| C | −0.56 | 0.00 | 0.00 | 0.00 | 0.00 | 0.00 | 0.00 | 0.00 | −0.32 | −0.01 | 0.00 | 0.00 | 0.01 | −0.21 |
| S | −0.31 | 0.00 | −0.01 | −0.13 | 0.00 | −0.28 | −0.28 | 0.00 | 0.05 | 0.00 | 0.00 | −0.02 | −0.18 | 0.59 |
| T | 0.00 | 0.00 | 0.00 | −0.02 | 0.00 | −0.15 | 0.99 | 0.00 | −0.01 | 0.00 | 0.00 | −0.75 | 0.00 | −0.01 |
| N | 0.00 | −0.43 | −0.72 | 0.00 | 0.00 | 1.86 | −0.04 | 0.00 | −0.29 | 0.00 | 0.00 | 0.00 | 0.34 | 0.04 |
| Q | 0.00 | 0.00 | 0.00 | 0.00 | 0.34 | 0.00 | 0.00 | 0.00 | −0.01 | −0.02 | 1.61 | 0.00 | −0.01 | 0.00 |
| Y | 0.00 | −0.37 | −0.34 | 0.00 | 0.00 | −0.86 | 0.00 | 0.00 | 1.07 | 0.00 | 0.00 | 0.00 | −0.50 | 0.00 |
| H | −0.61 | −0.02 | −0.01 | 0.00 | −0.01 | −0.16 | 0.00 | 0.00 | −0.18 | −0.78 | −0.04 | 0.00 | −0.09 | 0.00 |
| R | 1.83 | 0.00 | 0.00 | 0.00 | −0.42 | 0.00 | 0.00 | 0.00 | −0.01 | −0.20 | −0.92 | −0.18 | −0.02 | −0.27 |
| K | 0.00 | 0.00 | 0.00 | 0.00 | 0.00 | −0.08 | 0.00 | 0.00 | 0.00 | 0.00 | −0.09 | −0.88 | −0.34 | 0.00 |
| D | 0.00 | 2.73 | 3.07 | 0.01 | 0.00 | 0.31 | 0.00 | −0.65 | −0.09 | 0.00 | 0.00 | 0.00 | 0.81 | 0.00 |
| E | 0.00 | 0.05 | −0.20 | 0.15 | 0.00 | 0.00 | 0.00 | −0.01 | 0.00 | 0.00 | 0.04 | 0.00 | 0.01 | 0.00 |

Frame3

| | 29 | 30 | 31 | 32 | 33 | 34 | 35 | 36 | 37 | 38 | 39 | 40 | 41 |
|---|---|---|---|---|---|---|---|---|---|---|---|---|---|
| P | −0.65 | 0.00 | 0.22 | 0.00 | 0.00 | −0.14 | −0.02 | 0.00 | 0.00 | 0.00 | 0.00 | −0.06 | −0.06 |
| A | 0.00 | 0.00 | 0.01 | −0.15 | −0.06 | −0.26 | 1.28 | −0.61 | 0.00 | 0.00 | −0.01 | 0.19 | 1.23 |
| G | 0.00 | 0.00 | 0.00 | −0.74 | −0.90 | 0.00 | 0.00 | −2.19 | 0.00 | −0.01 | −0.13 | −0.04 | −0.05 |
| M | 0.00 | 0.00 | 0.00 | −0.01 | 0.00 | −0.01 | 0.00 | 0.00 | 0.00 | 0.00 | 0.00 | 0.00 | 0.00 |
| I | −0.12 | 0.19 | 0.00 | 0.00 | −0.01 | −0.28 | 0.00 | 0.13 | −0.01 | 0.00 | 0.00 | 0.00 | 0.00 |
| L | 1.87 | 0.00 | −0.16 | 0.00 | 0.00 | 0.00 | 0.00 | 0.00 | 0.00 | 0.00 | 0.00 | 0.00 | 0.00 |
| V | 0.03 | 0.01 | 0.00 | −0.60 | −0.79 | 0.00 | −0.50 | 2.60 | 0.00 | 0.00 | 0.00 | 0.43 | −0.26 |
| F | −0.14 | 0.00 | 0.00 | 0.00 | 0.00 | 0.00 | 0.00 | 0.31 | −0.36 | 0.11 | −0.11 | 0.00 | 0.00 |
| W | 0.00 | 0.00 | 0.00 | −0.01 | 0.00 | 0.00 | 0.00 | 0.00 | 0.00 | 0.00 | −0.05 | 0.00 | 0.00 |
| C | 0.00 | 0.00 | 0.00 | 0.00 | 0.00 | 0.00 | 0.00 | 0.00 | −0.69 | −0.41 | 2.82 | 0.00 | 0.00 |
| S | 0.00 | 0.00 | −0.03 | 0.00 | 0.00 | −0.18 | −0.16 | −0.02 | −0.14 | −0.03 | −0.69 | −0.08 | −0.12 |
| T | −0.01 | 0.06 | 0.01 | 0.00 | 0.00 | 0.95 | −0.40 | −0.02 | −0.01 | −0.01 | 0.00 | −0.23 | −0.57 |
| N | 0.00 | 0.09 | 0.00 | 0.00 | −0.15 | −0.09 | 0.00 | 0.00 | −0.78 | −0.60 | −0.01 | 0.00 | 0.00 |
| Q | 0.00 | 0.06 | −0.02 | −0.03 | 0.00 | 0.00 | 0.00 | 0.00 | 0.00 | 0.00 | 0.00 | 0.00 | −0.01 |
| Y | −0.01 | −0.02 | 0.00 | 0.00 | −0.23 | 0.00 | 0.00 | 0.00 | 2.83 | 1.72 | −0.42 | 0.00 | 0.00 |
| H | −0.82 | 0.00 | 0.00 | 0.00 | −0.04 | 0.00 | 0.00 | 0.00 | −0.70 | −0.56 | 0.00 | 0.00 | 0.00 |
| R | −0.14 | −0.53 | −0.03 | −0.02 | 0.00 | 0.00 | −0.01 | 0.00 | −0.02 | −0.01 | −1.40 | 0.00 | 0.00 |
| K | 0.00 | −0.39 | 0.00 | −0.19 | 0.00 | 0.00 | 0.00 | 0.00 | 0.00 | 0.00 | 0.00 | 0.00 | 0.00 |
| D | 0.00 | 0.00 | 0.00 | 0.04 | 2.47 | 0.00 | 0.00 | −0.18 | −0.11 | −0.20 | 0.00 | −0.21 | 0.00 |
| E | 0.00 | 0.52 | 0.00 | 1.72 | −0.30 | 0.00 | −0.20 | −0.02 | 0.00 | 0.00 | 0.00 | 0.00 | −0.15 |

CDR3

| | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| P | −0.03 | 0.00 | −0.71 | 1.42 | 0.00 | 0.00 | −0.66 | 0.00 | 0.00 | 0.00 | 0.00 | 0.00 | 0.00 | 0.00 | 0.00 |
| A | 0.82 | −0.13 | −1.04 | 0.00 | 0.00 | 0.00 | 0.00 | −0.01 | 0.00 | −0.14 | 0.00 | 0.00 | 0.00 | 0.00 | 0.00 |
| G | 0.02 | −0.98 | 0.00 | 0.00 | −0.02 | 0.00 | 0.00 | 0.09 | 0.00 | −0.69 | 0.00 | 0.00 | 0.00 | 0.00 | 0.00 |
| M | 0.00 | 0.00 | 0.00 | 0.00 | 0.00 | 0.03 | −0.09 | 0.00 | 0.00 | 0.00 | 0.00 | 0.00 | 0.00 | 0.00 | 0.00 |
| I | 0.00 | 0.00 | −0.19 | 0.00 | 0.64 | 1.66 | 0.00 | 0.12 | 0.00 | 0.00 | 0.00 | 0.00 | 0.00 | 0.00 | 0.00 |
| L | −0.02 | 0.00 | 0.00 | −0.59 | −0.01 | −0.11 | 0.57 | −0.02 | 0.01 | −0.02 | 0.00 | 0.00 | 0.00 | 0.00 | 0.00 |
| V | −0.41 | −0.99 | 0.00 | 0.00 | 0.01 | −0.49 | 0.01 | −0.37 | 0.00 | −0.80 | 0.00 | 0.00 | 0.00 | 0.00 | 0.00 |
| F | 0.00 | 0.00 | 0.00 | −0.02 | −0.01 | 0.46 | 0.00 | 0.10 | 0.02 | 0.00 | 0.00 | 0.00 | 0.00 | 0.00 | 0.00 |
| W | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| C | 0.00 | 0.00 | 0.00 | 0.00 | 0.01 | −0.01 | 0.00 | 0.00 | 0.28 | 0.00 | 0.00 | 0.00 | 0.00 | 0.00 | 0.00 |
| S | 0.00 | 0.00 | −0.58 | −0.20 | −0.77 | −0.17 | 0.00 | −0.02 | 0.13 | 0.00 | 0.00 | 0.00 | 0.00 | 0.00 | 0.00 |
| T | −0.12 | 0.00 | 2.58 | −0.27 | −0.11 | −0.98 | 0.00 | 0.00 | 0.00 | −0.01 | 0.00 | 0.00 | 0.00 | 0.00 | 0.00 |
| N | 0.00 | 0.00 | −0.07 | 0.00 | 0.63 | −0.39 | 0.00 | 0.00 | 0.28 | 0.00 | 0.00 | 0.00 | 0.00 | 0.00 | 0.00 |
| Q | 0.00 | −0.03 | 0.00 | −0.01 | 0.00 | 0.00 | 0.30 | 0.00 | 0.00 | −0.03 | 0.00 | 0.00 | 0.00 | 0.00 | 0.00 |
| Y | 0.00 | 0.00 | 0.00 | 0.00 | −0.24 | −0.01 | 0.00 | 0.00 | −1.21 | 0.00 | 0.00 | 0.00 | 0.00 | 0.00 | 0.00 |
| H | 0.00 | 0.00 | 0.00 | −0.28 | 0.00 | 0.00 | 0.00 | 0.00 | 0.28 | 0.00 | 0.00 | 0.00 | 0.00 | 0.00 | 0.00 |
| R | −0.01 | 0.00 | 0.00 | −0.04 | 0.00 | 0.00 | −0.13 | 0.00 | 0.00 | −0.01 | 0.00 | 0.00 | 0.00 | 0.00 | 0.00 |
| K | 0.00 | −0.13 | 0.00 | 0.00 | −0.15 | 0.00 | 0.00 | 0.00 | 0.00 | −0.30 | 0.00 | 0.00 | 0.00 | 0.00 | 0.00 |
| D | 0.00 | −0.01 | 0.00 | 0.00 | 0.02 | 0.00 | 0.00 | 0.10 | 0.21 | 0.07 | 0.00 | 0.00 | 0.00 | 0.00 | 0.00 |
| E | −0.25 | 2.27 | 0.00 | 0.00 | 0.00 | 0.00 | 0.00 | 0.00 | 0.00 | 1.93 | 0.00 | 0.00 | 0.00 | 0.00 | 0.00 |

CDR3

| | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|
| P | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| A | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| G | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| M | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| I | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| L | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| V | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| F | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| W | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| C | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| S | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| T | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| N | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| Q | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| Y | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| H | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| R | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| K | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| D | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| E | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |

SR6 post- minus pre- affinity maturation a.a. profile
Frame4

| | 1 | 2 | 3 | 4 | 5 | 6 | 7 | 8 | 9 | 10 | 11 | 12 | 13 |
|---|---|---|---|---|---|---|---|---|---|---|---|---|---|
| P | −0.01 | 0.00 | 0.00 | 0.00 | 0.08 | 0.00 | −0.15 | 0.08 | 0.00 | −0.78 | 0.00 | −1.11 | 0.00 |
| A | 0.04 | −0.01 | 0.00 | 0.01 | 0.00 | −0.09 | −0.06 | 0.00 | 0.04 | −0.05 | 0.03 | 0.02 | −0.02 |
| G | 0.00 | 0.00 | −0.13 | 0.16 | 0.00 | 0.73 | 0.00 | 0.00 | 0.02 | 0.00 | −0.01 | 0.00 | 0.01 |
| M | 0.00 | 0.00 | 0.00 | 0.00 | 0.00 | 0.00 | 0.00 | 0.00 | 0.00 | 0.00 | 0.00 | 0.00 | −0.03 |
| I | −0.24 | 0.01 | 0.00 | 0.00 | 0.00 | −0.01 | −0.02 | 0.00 | 0.01 | 0.02 | −0.05 | 0.00 | −0.03 |
| L | 0.00 | −0.01 | −0.13 | 0.00 | 0.05 | 0.00 | 0.00 | 0.21 | −0.04 | 0.00 | 0.00 | −0.01 | 0.00 |
| V | 0.01 | 0.00 | 0.00 | 0.02 | 0.00 | −0.14 | 0.00 | 0.00 | 0.71 | 0.00 | 1.10 | −0.01 | −1.33 |
| F | −0.02 | −0.21 | 0.00 | 0.00 | 0.00 | 0.00 | 0.00 | 0.00 | −0.03 | 0.00 | −1.04 | 0.00 | 0.00 |
| W | 0.00 | 0.00 | 1.97 | 0.00 | 0.00 | −0.01 | 0.00 | 0.00 | 0.00 | 0.00 | 0.00 | 0.00 | 0.00 |
| C | −0.02 | −0.09 | −0.12 | −0.04 | 0.00 | −0.09 | 0.00 | 0.00 | 0.00 | 0.00 | 0.00 | −0.01 | −0.05 |
| S | 1.03 | −0.07 | −0.03 | −0.03 | 0.00 | −0.07 | −0.30 | 0.00 | −0.66 | −0.01 | −0.01 | 1.11 | 1.31 |
| T | 0.76 | 0.00 | 0.00 | 0.00 | 0.00 | 0.00 | 0.73 | 0.00 | 0.00 | 0.82 | 0.00 | 0.02 | 0.01 |
| N | −1.62 | −0.19 | 0.00 | 0.00 | 0.00 | 0.00 | −0.20 | 0.00 | 0.00 | 0.02 | 0.00 | 0.00 | 0.00 |
| Q | 0.00 | 0.00 | −0.01 | 0.00 | 0.32 | 0.00 | 0.00 | 0.65 | 0.00 | −0.01 | 0.00 | −0.01 | 0.00 |
| Y | 0.29 | 0.78 | 0.00 | −0.01 | 0.00 | 0.00 | −0.01 | 0.00 | −0.01 | 0.00 | −0.01 | 0.01 | 0.00 |
| H | 0.13 | −0.06 | 0.00 | 0.00 | −0.16 | 0.00 | 0.00 | −0.04 | 0.00 | 0.00 | 0.00 | 0.00 | 0.00 |
| R | −0.01 | 0.00 | −1.52 | −0.03 | −0.24 | −0.13 | 0.00 | −0.67 | 0.00 | 0.00 | 0.00 | 0.00 | 0.13 |
| K | −0.14 | 0.00 | −0.02 | 0.00 | −0.06 | 0.00 | 0.00 | −0.22 | 0.00 | 0.00 | 0.00 | 0.00 | 0.00 |
| D | −0.21 | −0.15 | 0.00 | −0.11 | 0.00 | −0.18 | 0.00 | 0.00 | −0.04 | 0.00 | −0.01 | 0.00 | 0.00 |
| E | 0.00 | 0.00 | −0.01 | 0.00 | 0.00 | −0.01 | 0.00 | 0.00 | 0.00 | 0.00 | 0.00 | 0.00 | 0.00 |

Frame1

| | 1 | 2 | 3 | 4 | 5 | 6 | 7 | 8 | 9 | 10 | 11 | 12 | 13 |
|---|---|---|---|---|---|---|---|---|---|---|---|---|---|
| P | 0.00 | 0.00 | 0.00 | 0.00 | 0.00 | 0.00 | −1.82 | 0.00 | 0.00 | 0.00 | 0.00 | 0.00 | −0.57 |
| A | 0.13 | −0.27 | 0.00 | 0.00 | 0.13 | −0.18 | −0.14 | 0.00 | 0.00 | 0.03 | 0.00 | −0.38 | 0.00 |
| G | −0.86 | −0.57 | −0.30 | 0.00 | −0.61 | −1.36 | 0.00 | 1.63 | 1.84 | −0.16 | 0.00 | −0.30 | 0.00 |
| M | 0.00 | 0.00 | 0.00 | 0.00 | 0.00 | 0.00 | 0.00 | 0.00 | 0.00 | 0.00 | 0.03 | 0.00 | 0.00 |
| I | 0.00 | −0.27 | 0.00 | −1.18 | −0.16 | 0.03 | −0.88 | −0.30 | 0.00 | 0.00 | −1.80 | −0.14 | 0.00 |
| L | 0.00 | 0.00 | 0.11 | 2.92 | 0.48 | 0.00 | 0.27 | 0.00 | 0.00 | 0.00 | 1.24 | 0.03 | −1.23 |
| V | 0.67 | 1.69 | 0.00 | −0.27 | 1.18 | 4.72 | 0.00 | −0.30 | 0.16 | 0.03 | 0.03 | 1.39 | 0.00 |
| F | 0.00 | 0.00 | 0.00 | −0.35 | 0.10 | 0.00 | 0.10 | 0.00 | 0.00 | 0.00 | 1.52 | 0.01 | 0.00 |
| W | 0.03 | 0.00 | 0.03 | 0.00 | 0.00 | 0.00 | 0.00 | 0.00 | 0.00 | 0.00 | 0.00 | 0.00 | 0.00 |
| C | 0.00 | 0.00 | 0.00 | 0.00 | 0.00 | 0.00 | 0.00 | 0.00 | 0.00 | 0.25 | 0.00 | 0.00 | 0.00 |
| S | 0.03 | −0.61 | −0.61 | −0.21 | −0.57 | 0.03 | 1.71 | 0.00 | 0.00 | 0.22 | −0.78 | 0.00 | −0.30 |
| T | 0.00 | 0.00 | −0.27 | −0.30 | −0.30 | 0.00 | 1.03 | 0.00 | 0.00 | 0.00 | 0.00 | 0.00 | 0.00 |
| N | 0.00 | 0.03 | 0.00 | 0.00 | 0.03 | 0.00 | 0.00 | 0.00 | 0.00 | 0.00 | 0.00 | 0.00 | 0.00 |
| Q | 5.30 | 0.00 | −0.12 | −0.61 | 0.00 | 0.51 | 0.00 | 0.00 | 0.00 | 0.00 | 0.00 | 0.00 | 0.97 |
| Y | 0.03 | 0.00 | 0.00 | 0.00 | 0.00 | 0.00 | 0.03 | 0.00 | 0.00 | 0.00 | 0.03 | 0.00 | 0.00 |
| H | −0.24 | 0.00 | 0.01 | 0.00 | 0.00 | 0.00 | 0.00 | 0.00 | 0.00 | 0.00 | 0.00 | 0.00 | −0.26 |
| R | 0.03 | 0.00 | 1.14 | 0.00 | 0.00 | −0.91 | −0.30 | −1.08 | −1.75 | −0.45 | −0.27 | 0.00 | 1.57 |
| K | 0.13 | 0.00 | 0.00 | 0.00 | 0.00 | −0.14 | 0.00 | 0.00 | 0.00 | 0.00 | 0.00 | 0.00 | 0.13 |
| D | 0.70 | 0.00 | 0.00 | 0.00 | 0.00 | −0.35 | 0.00 | 0.00 | 0.03 | 0.06 | 0.00 | −0.61 | 0.00 |
| E | −5.98 | 0.00 | 0.00 | 0.00 | −0.27 | −2.35 | 0.00 | 0.06 | −0.27 | 0.00 | 0.00 | 0.00 | −0.30 |

Frame1

| | 14 | 15 | 16 | 17 | 18 | 19 | 20 | 21 | 22 | 23 | 24 | 25 | 26 |
|---|---|---|---|---|---|---|---|---|---|---|---|---|---|
| P | 0.03 | 0.00 | 0.00 | −1.36 | −0.27 | 0.00 | 0.00 | 0.00 | 0.00 | −0.30 | 0.00 | −1.18 | 0.00 |
| A | 0.58 | 0.00 | −0.18 | −0.81 | 0.00 | 0.00 | 0.00 | 0.00 | 0.00 | −1.09 | 0.88 | 0.06 | 0.00 |
| G | −0.27 | 1.36 | −0.51 | 0.00 | 0.00 | 0.00 | 0.00 | −0.56 | 0.00 | 0.06 | 0.00 | 0.00 | 0.96 |
| M | 0.00 | 0.00 | 0.00 | 0.03 | −0.42 | 0.00 | 0.00 | 0.00 | 0.00 | 0.00 | 0.00 | 0.00 | 0.00 |
| I | 0.00 | 0.00 | 0.00 | 0.00 | 0.00 | 0.00 | 0.14 | 0.16 | 0.00 | 0.00 | 0.00 | 0.00 | 0.00 |
| L | 0.00 | 0.00 | 0.00 | −0.30 | 2.35 | −0.21 | 1.35 | 0.00 | 0.00 | 0.00 | 0.03 | 0.00 | 0.00 |
| V | 0.29 | 0.00 | −1.69 | 0.03 | −0.30 | 0.00 | −0.30 | 0.00 | 0.00 | 0.56 | −0.11 | 0.00 | −0.30 |
| F | 0.00 | 0.00 | 0.00 | 0.00 | 0.00 | 0.00 | −0.58 | 0.00 | −0.27 | 0.00 | 0.00 | 0.13 | 0.00 |
| W | 0.00 | 0.00 | 0.00 | 0.00 | 0.00 | 0.00 | 0.00 | 0.00 | 0.00 | 0.00 | 0.00 | 0.00 | 0.00 |
| C | 0.00 | 0.03 | 0.03 | 0.03 | 0.00 | −0.62 | 0.00 | −0.43 | 1.99 | 0.00 | 0.00 | 0.00 | −0.30 |
| S | −0.24 | −0.81 | 0.00 | 1.81 | 0.00 | 0.19 | −0.61 | 0.77 | −1.15 | 0.10 | −0.27 | 0.34 | −0.18 |
| T | −0.48 | 0.00 | 0.00 | 0.57 | 0.00 | 0.00 | 0.00 | 0.03 | 0.03 | 0.59 | −0.22 | 0.08 | 0.00 |
| N | 0.00 | 0.00 | 0.11 | 0.00 | 0.00 | 0.00 | 0.00 | −0.51 | 0.00 | 0.00 | 0.00 | 0.00 | 0.00 |
| Q | 0.00 | 0.00 | 0.00 | 0.00 | −1.48 | 0.00 | 0.00 | 0.00 | 0.00 | 0.00 | 0.00 | 0.00 | 0.00 |
| Y | 0.00 | 0.00 | −0.21 | 0.00 | 0.00 | 0.00 | 0.00 | 0.00 | 0.00 | 0.00 | 0.00 | 0.57 | 0.00 |
| H | 0.00 | 0.00 | 0.06 | 0.00 | 0.00 | −1.02 | 0.00 | 0.00 | 0.00 | 0.00 | 0.00 | 0.00 | 0.00 |
| R | 0.00 | 0.00 | 0.00 | 0.00 | 0.13 | 1.66 | 0.00 | 0.54 | −0.61 | 0.00 | 0.00 | 0.00 | 0.00 |
| K | 0.00 | 0.00 | 0.00 | 0.00 | 0.00 | 0.00 | 0.00 | 0.00 | 0.00 | 0.00 | 0.00 | 0.00 | 0.00 |
| D | 0.00 | −0.27 | 2.80 | 0.00 | 0.00 | 0.00 | 0.00 | 0.00 | 0.00 | 0.10 | 0.00 | 0.00 | −0.18 |
| E | 0.10 | −0.30 | −0.42 | 0.00 | 0.00 | 0.00 | 0.00 | 0.00 | 0.00 | 0.00 | −0.30 | 0.00 | 0.00 |

CDR1

| | 1 | 2 | 3 | 4 | 5 | 6 | 7 | 8 | 9 | 10 |
|---|---|---|---|---|---|---|---|---|---|---|
| P | 0.00 | 0.00 | −0.30 | 0.00 | 0.00 | 0.00 | 0.00 | 0.00 | 0.00 | 0.00 |
| A | −0.30 | 0.00 | 0.00 | −0.54 | 0.00 | 0.00 | 0.00 | 0.00 | 0.00 | 0.00 |
| G | −0.51 | −1.15 | 0.00 | −0.45 | 0.00 | 0.00 | 0.03 | 0.00 | 0.00 | 0.00 |
| M | 0.00 | 0.00 | 0.00 | 0.00 | 0.00 | 0.00 | 0.00 | 0.00 | 0.00 | 0.00 |
| I | 0.00 | 0.00 | 0.00 | −0.21 | 0.06 | 0.00 | 0.00 | 0.00 | 0.00 | 0.00 |
| L | 0.00 | 0.00 | −2.12 | 0.03 | 2.01 | −0.57 | 0.03 | 0.00 | 0.00 | 0.00 |
| V | −1.21 | 0.00 | 0.00 | 1.61 | −0.57 | 0.00 | 0.00 | 0.00 | 0.00 | 0.00 |
| F | 0.00 | 0.00 | 0.00 | 0.03 | −0.08 | 0.00 | 0.03 | 0.00 | 0.00 | 0.00 |
| W | 0.00 | −0.30 | 0.00 | 0.00 | 0.00 | 2.30 | 0.00 | 0.00 | 0.00 | 0.00 |
| C | 0.00 | 3.61 | 0.03 | 0.00 | 0.00 | 0.06 | −0.24 | 0.00 | 0.00 | 0.00 |
| S | 0.03 | −0.42 | 0.00 | 0.00 | −1.15 | 0.00 | −1.18 | 0.00 | 0.00 | 0.00 |
| T | 0.00 | 0.00 | 0.00 | 0.00 | 0.00 | 0.00 | 0.00 | 0.00 | 0.00 | 0.00 |
| N | 0.41 | 0.00 | −0.18 | 0.00 | 0.00 | 0.00 | 0.00 | 0.00 | 0.00 | 0.00 |
| Q | 0.00 | 0.00 | −0.08 | 0.00 | −0.30 | 0.00 | 0.00 | 0.00 | 0.00 | 0.00 |
| Y | −0.91 | −0.54 | −0.72 | 0.00 | 0.00 | 0.00 | 0.00 | 0.00 | 0.00 | 0.00 |
| H | 0.00 | 0.00 | 5.90 | 0.00 | 0.00 | 0.00 | −0.91 | 0.00 | 0.00 | 0.00 |
| R | 0.00 | −1.20 | −1.96 | 0.00 | 0.00 | −1.79 | 2.23 | 0.00 | 0.00 | 0.00 |
| K | 0.00 | 0.00 | 0.00 | 0.00 | 0.00 | 0.00 | 0.00 | 0.00 | 0.00 | 0.00 |
| D | 2.33 | 0.00 | −0.57 | −0.48 | 0.03 | 0.00 | 0.00 | 0.00 | 0.00 | 0.00 |
| E | 0.16 | 0.00 | 0.00 | 0.00 | 0.00 | 0.00 | 0.00 | 0.00 | 0.00 | 0.00 |

Frame2

| | 1 | 2 | 3 | 4 | 5 | 6 | 7 | 8 | 9 | 10 | 11 | 12 | 13 | 14 | 15 | 16 | 17 | 18 | 19 |
|---|---|---|---|---|---|---|---|---|---|---|---|---|---|---|---|---|---|---|---|
| P | 0.00 | 0.00 | 0.00 | 0.00 | 0.03 | 0.03 | 0.00 | −0.41 | 0.00 | 0.00 | 0.00 | 0.16 | 0.00 | 0.00 | 0.00 | 0.00 | 0.00 | 0.00 | 0.00 |
| A | 0.00 | 0.10 | 0.00 | 0.00 | 0.00 | 0.00 | 0.47 | 0.03 | −0.30 | 0.00 | 0.03 | 0.00 | 0.00 | 0.00 | −0.27 | 2.54 | 2.39 | 0.00 | 0.00 |
| G | 0.00 | 0.11 | 0.00 | 0.00 | 0.00 | 0.00 | 0.00 | 0.00 | 2.07 | 0.00 | 0.08 | 0.00 | −0.57 | 0.00 | 0.03 | 0.10 | −0.30 | 0.00 | 0.08 |
| M | 2.40 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.03 | 0.00 |
| I | 0.16 | 0.00 | 0.00 | −0.24 | 0.00 | 0.00 | 0.00 | 0.00 | 0.00 | −0.85 | 0.00 | 0.00 | 0.00 | −0.30 | −0.22 | 0.00 | 0.00 | 1.50 | −0.61 |
| L | 0.44 | 0.00 | −0.30 | −3.41 | 0.00 | −1.96 | 0.00 | 0.16 | 0.00 | 0.03 | 0.00 | 0.89 | 0.00 | −1.21 | 0.03 | 0.00 | 0.00 | 0.00 | 0.00 |
| V | −0.34 | 0.03 | 0.00 | −0.30 | 0.00 | 0.00 | −0.26 | 0.00 | −0.30 | 0.00 | −0.26 | 0.00 | 0.25 | 0.00 | 1.64 | −0.30 | −0.61 | 0.08 | 0.00 |
| F | 0.00 | 0.00 | 0.00 | 5.10 | 0.00 | 0.00 | 0.00 | 0.00 | 0.00 | 0.00 | 0.00 | 0.00 | 0.00 | 2.84 | −0.30 | 0.00 | 0.00 | −0.61 | 0.00 |
| W | 0.00 | 0.00 | 3.78 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| C | 0.00 | 0.00 | −0.14 | 0.00 | −0.18 | 0.00 | 0.00 | 0.00 | 0.00 | 0.00 | 0.00 | 0.43 | 0.00 | −0.30 | 0.00 | 0.00 | 0.00 | 0.00 | −0.88 |
| S | 0.00 | 0.00 | 0.00 | −0.88 | 0.03 | 0.00 | −0.29 | 0.32 | 0.00 | 0.00 | 0.00 | 0.10 | 0.00 | −0.91 | 0.00 | −0.91 | 0.00 | 0.06 | 2.92 |
| T | −1.75 | 0.00 | 0.00 | 0.00 | 0.00 | 0.00 | −0.05 | 0.16 | 0.00 | −0.14 | 0.00 | 0.00 | 0.00 | 0.00 | 0.00 | −1.45 | −0.57 | −0.19 | 0.00 |
| N | 0.00 | 0.00 | 0.00 | 0.00 | 0.00 | 0.00 | 0.00 | 0.00 | 0.00 | −1.34 | 0.00 | 0.00 | 0.00 | 0.00 | 0.00 | 0.00 | 0.00 | −0.88 | −0.91 |
| Q | 0.00 | 0.00 | 0.00 | 0.00 | 0.00 | 3.85 | 0.00 | −0.27 | 0.00 | 0.03 | 0.03 | 0.00 | −0.27 | 0.00 | 0.00 | 0.00 | 0.00 | 0.00 | 0.00 |
| Y | 0.00 | 0.00 | 0.00 | −0.27 | 0.00 | 0.00 | 0.00 | 0.00 | 0.00 | 0.00 | 0.00 | 0.00 | 0.00 | −0.11 | 0.00 | 0.00 | 0.00 | 0.00 | 0.00 |
| H | 0.00 | 0.00 | 0.00 | 0.00 | −0.57 | −1.18 | 0.00 | −0.02 | 0.00 | 0.00 | 0.00 | 0.43 | 0.00 | 0.00 | 0.00 | 0.00 | 0.00 | 0.00 | 0.00 |
| R | −0.30 | 0.03 | −3.33 | 0.00 | 0.69 | −0.99 | 0.00 | 0.03 | −0.85 | −0.32 | 0.00 | −2.00 | 0.00 | 0.00 | 0.00 | 0.00 | 0.00 | 0.00 | −0.61 |
| K | −0.61 | 0.00 | 0.00 | 0.00 | 0.00 | 0.22 | 0.00 | 0.00 | 0.00 | 3.05 | −0.81 | 0.00 | −0.24 | 0.00 | 0.00 | 0.00 | 0.00 | 0.00 | 0.00 |
| D | 0.00 | 0.00 | 0.00 | 0.00 | 0.00 | 0.00 | 0.00 | 0.00 | 0.00 | 0.00 | −0.88 | 0.00 | −0.88 | 0.00 | −0.91 | 0.00 | 0.00 | 0.00 | 0.00 |
| E | 0.00 | −0.27 | 0.00 | 0.00 | 0.00 | 0.03 | 0.13 | 0.00 | −0.62 | −0.46 | 1.81 | 0.00 | 1.71 | 0.00 | 0.00 | 0.03 | −0.91 | 0.00 | 0.00 |

CDR2

| | 1 | 2 | 3 | 4 | 5 | 6 | 7 | 8 | 9 | 10 |
|---|---|---|---|---|---|---|---|---|---|---|
| P | 0.00 | 0.00 | 0.00 | −0.54 | 0.00 | 0.00 | 0.00 | 0.00 | 0.00 | 0.00 |
| A | 0.00 | −0.27 | 0.00 | 0.00 | 0.00 | 0.00 | 0.00 | 0.00 | 0.00 | 0.00 |
| G | 0.00 | −2.39 | 0.72 | 0.00 | 0.00 | 0.00 | 0.00 | 0.00 | 0.00 | 0.00 |
| M | 0.00 | 0.00 | 0.00 | −0.35 | 0.00 | 0.00 | 0.00 | 0.00 | 0.00 | 0.00 |
| I | −0.54 | 0.00 | 0.00 | 0.00 | −0.61 | 0.00 | 0.00 | 0.00 | 0.00 | 0.00 |
| L | 1.66 | 0.00 | 0.00 | −0.07 | −3.64 | 0.00 | 0.00 | 0.00 | 0.00 | 0.00 |
| V | 0.03 | −1.21 | 0.00 | 0.00 | −4.24 | 0.00 | 0.00 | 0.00 | 0.00 | 0.00 |
| F | −0.27 | 0.00 | 0.00 | 0.00 | 11.26 | 0.00 | 0.00 | 0.00 | 0.00 | 0.00 |
| W | 0.00 | 0.00 | 0.00 | 0.00 | 0.00 | 0.00 | 0.00 | 0.00 | 0.00 | 0.00 |
| C | 0.00 | 0.00 | −0.54 | 0.00 | −0.30 | 0.00 | 0.00 | 0.00 | 0.00 | 0.00 |
| S | −0.88 | 0.00 | 0.13 | 0.00 | −2.12 | 0.00 | 0.00 | 0.00 | 0.00 | 0.00 |
| T | 0.00 | 0.00 | 0.00 | 0.00 | 0.00 | 0.00 | 0.00 | 0.00 | 0.00 | 0.00 |
| N | 0.00 | 0.06 | 0.00 | 0.00 | 0.00 | 0.00 | 0.00 | 0.00 | 0.00 | 0.00 |
| Q | 0.00 | 0.00 | 0.00 | 0.82 | 0.00 | 0.00 | 0.00 | 0.00 | 0.00 | 0.00 |
| Y | 0.00 | −0.27 | 0.00 | 0.00 | −0.35 | 0.00 | 0.00 | 0.00 | 0.00 | 0.00 |
| H | 0.00 | 0.00 | 0.00 | 0.00 | 0.00 | 0.00 | 0.00 | 0.00 | 0.00 | 0.00 |
| R | 0.00 | 0.00 | 0.00 | 0.14 | 0.00 | 0.00 | 0.00 | 0.00 | 0.00 | 0.00 |
| K | 0.00 | 0.00 | 0.00 | 0.00 | 0.00 | 0.00 | 0.00 | 0.00 | 0.00 | 0.00 |
| D | 0.00 | 4.05 | −0.30 | 0.00 | 0.00 | 0.00 | 0.00 | 0.00 | 0.00 | 0.00 |
| E | 0.00 | 0.03 | 0.00 | 0.00 | 0.00 | 0.00 | 0.00 | 0.00 | 0.00 | 0.00 |

Frame3

| | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| P | −0.48 | 0.00 | 0.00 | 0.00 | 0.00 | −0.61 | 0.00 | 0.00 | 0.00 | 0.00 | 0.00 | 0.00 | 0.00 | −0.88 |
| A | 0.92 | 0.00 | 0.00 | 1.34 | 0.03 | 0.22 | 0.01 | 0.00 | 0.06 | 0.00 | 0.00 | 0.00 | 0.00 | 0.00 |
| G | 0.00 | 0.00 | 0.00 | 0.00 | −0.21 | 0.00 | 0.03 | 0.00 | 2.91 | 0.00 | 0.00 | −0.10 | 0.00 | 0.00 |
| M | 0.00 | 0.00 | 0.00 | 0.00 | 0.00 | 0.00 | 0.00 | 0.00 | 0.00 | 0.00 | 0.00 | 0.00 | 0.06 | 0.00 |
| I | −0.13 | −0.54 | 0.00 | 0.00 | 0.00 | 0.00 | −0.24 | 0.00 | 0.00 | 0.00 | −0.21 | 0.32 | 2.57 | 0.00 |
| L | 0.00 | 0.00 | 0.00 | 0.00 | 0.00 | −0.45 | 0.06 | 0.25 | 0.00 | 0.00 | −0.21 | 0.00 | 0.06 | 0.00 |
| V | 0.00 | 0.00 | 0.00 | −0.27 | −0.35 | 0.00 | 0.40 | 0.00 | −0.61 | 0.00 | −0.27 | 0.00 | 0.86 | 0.00 |
| F | 0.00 | 0.00 | −0.78 | 0.00 | 0.00 | 0.00 | 0.00 | 0.00 | 0.00 | 0.00 | 2.54 | 0.00 | −2.42 | 0.29 |
| W | 0.00 | 0.00 | 0.00 | 0.00 | 0.00 | 0.00 | 0.00 | 0.00 | 0.00 | −1.52 | 0.00 | 0.00 | 0.00 | 0.00 |
| C | 0.00 | 0.00 | −0.54 | 0.00 | 0.00 | 0.00 | 0.00 | −0.11 | −0.27 | 0.00 | −0.57 | −0.21 | 0.00 | 0.00 |
| S | 0.46 | −0.58 | 0.00 | −0.18 | 0.00 | 0.72 | 0.00 | 0.13 | −1.79 | 0.00 | −0.72 | 0.28 | 0.00 | 0.95 |
| T | −0.81 | 0.22 | 0.00 | −0.42 | 0.00 | 0.11 | 0.00 | 0.00 | 0.03 | 0.00 | 0.00 | 0.00 | −0.53 | 0.03 |
| N | 0.03 | 3.26 | −0.91 | 0.00 | −0.54 | 0.00 | 0.00 | 0.00 | 0.00 | 0.00 | 0.00 | −0.18 | −0.61 | 0.00 |
| Q | 0.00 | 0.00 | 0.00 | 0.00 | 0.00 | 0.00 | 0.00 | 0.00 | 0.00 | −0.54 | 0.00 | 0.00 | 0.00 | 0.00 |
| Y | 0.00 | −0.88 | 3.69 | 0.00 | −0.24 | 0.00 | 0.00 | 0.00 | 0.00 | 0.00 | −0.56 | 0.00 | 0.00 | −0.38 |
| H | 0.00 | −0.30 | −1.45 | 0.00 | 0.06 | 0.00 | 0.00 | −0.96 | 0.00 | 0.00 | 0.00 | 0.00 | 0.00 | 0.00 |
| R | 0.00 | 0.00 | 0.00 | 0.00 | 0.00 | 0.00 | 0.00 | 0.69 | 0.00 | 2.06 | 0.00 | −0.11 | 0.00 | 0.00 |
| K | 0.00 | −0.61 | 0.00 | 0.00 | 0.00 | 0.00 | 0.00 | 0.00 | 0.00 | 0.00 | 0.00 | 0.00 | 0.00 | 0.00 |
| D | 0.00 | −0.57 | 0.00 | −0.48 | 0.67 | 0.00 | 0.00 | 0.00 | −0.34 | 0.00 | 0.00 | 0.00 | 0.00 | 0.00 |
| E | 0.00 | 0.00 | 0.00 | 0.00 | 0.57 | 0.00 | −0.27 | 0.00 | 0.00 | 0.00 | 0.00 | 0.00 | 0.00 | 0.00 |

Frame3

| | 15 | 16 | 17 | 18 | 19 | 20 | 21 | 22 | 23 | 24 | 25 | 26 | 27 | 28 |
|---|---|---|---|---|---|---|---|---|---|---|---|---|---|---|
| P | 0.00 | 0.00 | 0.01 | 0.00 | 0.00 | 0.00 | 0.06 | 0.00 | 0.00 | −0.30 | −0.30 | 0.00 | 0.00 | 0.00 |
| A | 0.40 | −0.30 | −0.08 | −0.22 | 0.00 | 0.00 | −0.82 | −0.81 | 0.00 | 0.00 | 0.00 | 0.03 | 0.00 | 0.00 |
| G | 0.00 | −0.30 | 0.00 | 0.03 | 0.03 | 0.00 | 0.00 | −1.79 | 0.00 | 0.00 | 0.00 | 0.00 | 0.00 | 0.03 |
| M | 0.00 | 0.00 | 0.00 | 0.00 | 0.52 | 0.00 | 0.01 | 0.00 | 0.00 | 0.00 | 0.00 | 3.34 | 0.00 | 0.00 |
| I | 0.00 | 0.00 | 0.00 | 0.00 | 0.00 | −0.78 | 0.00 | −0.02 | 0.00 | −1.48 | 0.00 | −0.53 | −0.45 | 0.19 |
| L | 0.00 | 0.00 | −0.42 | 0.00 | 0.03 | 0.00 | 0.00 | 0.00 | 0.00 | 2.93 | 0.43 | 0.27 | 0.00 | 0.00 |
| V | −0.29 | −0.18 | 0.00 | −0.14 | 0.00 | 0.00 | 0.00 | 2.78 | 0.00 | 0.00 | 0.00 | −0.27 | 0.00 | 0.00 |
| F | 0.00 | 0.00 | 0.00 | 0.00 | 0.00 | 0.00 | 0.00 | 0.41 | −0.70 | −0.30 | 0.00 | 0.00 | −0.30 | 0.00 |
| W | 0.00 | 0.00 | 0.00 | 0.00 | 0.00 | 0.00 | 0.00 | 0.00 | 0.00 | 0.00 | 0.00 | 0.00 | 0.00 | 0.00 |
| C | 0.00 | 0.00 | 0.00 | 0.00 | 0.00 | −0.27 | 0.00 | 0.00 | −0.80 | 0.00 | 0.00 | 0.00 | 0.00 | −0.47 |
| S | 0.06 | 0.00 | 0.71 | 0.19 | 0.00 | 0.38 | −0.42 | 0.00 | −0.18 | 0.00 | 0.00 | 0.00 | −0.90 | 2.53 |
| T | −0.18 | 0.00 | −0.23 | −0.03 | −0.11 | −0.02 | 1.12 | 0.00 | 0.00 | 0.00 | 0.00 | −0.78 | −0.11 | 0.00 |
| N | 0.00 | 0.03 | 0.00 | 0.00 | 0.70 | 0.53 | 0.00 | 0.00 | −0.83 | 0.00 | 0.00 | 0.00 | 3.71 | −0.13 |
| Q | 0.00 | 0.00 | 0.00 | 0.00 | −0.14 | 0.00 | 0.00 | 0.00 | 0.00 | 0.00 | 0.72 | 0.00 | 0.00 | 0.00 |
| Y | 0.00 | −0.30 | 0.00 | 0.00 | 0.00 | 2.02 | 0.00 | 0.00 | 4.42 | 0.00 | 0.00 | 0.00 | 0.11 | 0.00 |
| H | 0.00 | 0.00 | 0.00 | 0.00 | 0.00 | 0.06 | 0.00 | 0.00 | −2.04 | −0.57 | −0.02 | 0.00 | −0.24 | 0.00 |
| R | 0.00 | 0.00 | 0.00 | 0.00 | 0.49 | 0.00 | 0.00 | 0.00 | 0.03 | −0.27 | −0.89 | −0.54 | 0.03 | −2.19 |
| K | 0.00 | 0.00 | 0.00 | 0.00 | −2.42 | −0.21 | 0.03 | 0.00 | 0.00 | 0.00 | 0.06 | −1.52 | 0.03 | 0.00 |
| D | 0.00 | 0.83 | 0.00 | 0.00 | 0.00 | −1.71 | 0.00 | −0.57 | 0.10 | 0.00 | 0.00 | 0.00 | −1.92 | 0.03 |
| E | 0.00 | 0.22 | 0.00 | 0.17 | 0.90 | 0.00 | 0.00 | 0.00 | 0.00 | 0.00 | 0.00 | 0.00 | 0.03 | 0.00 |

Frame3

| | 29 | 30 | 31 | 32 | 33 | 34 | 35 | 36 | 37 | 38 | 39 | 40 | 41 |
|---|---|---|---|---|---|---|---|---|---|---|---|---|---|
| P | −1.05 | 0.00 | 0.48 | 0.00 | 0.00 | 0.03 | 0.00 | 0.00 | 0.00 | 0.00 | 0.00 | 0.00 | −0.30 |
| A | 0.00 | 0.00 | 0.03 | 0.03 | 0.00 | −0.19 | 0.78 | −0.53 | 0.00 | 0.00 | 0.00 | 0.75 | 1.15 |
| G | 0.00 | 0.00 | 0.00 | −0.96 | −1.52 | 0.00 | 0.03 | −2.09 | 0.00 | 0.00 | 0.03 | 0.10 | 0.00 |
| M | −0.08 | 0.00 | 0.03 | 0.00 | 0.00 | 0.00 | 0.00 | 0.00 | 0.00 | 0.00 | 0.00 | 0.00 | 0.00 |
| I | 0.00 | 0.09 | 0.00 | 0.00 | 0.00 | −0.24 | 0.00 | −0.26 | 0.00 | 0.03 | 0.00 | 0.00 | 0.00 |
| L | 3.56 | 0.00 | 0.38 | 0.00 | 0.00 | 0.00 | 0.00 | 0.00 | 0.00 | 0.00 | 0.00 | 0.00 | 0.00 |
| V | 0.00 | 0.00 | 0.00 | −0.45 | −0.30 | 0.00 | 0.00 | 3.29 | 0.00 | 0.00 | 0.00 | −0.42 | −0.24 |
| F | 0.00 | 0.00 | 0.00 | 0.00 | 0.00 | 0.00 | 0.00 | 0.03 | −0.24 | 0.21 | −0.88 | 0.00 | 0.00 |
| W | 0.00 | 0.00 | 0.00 | 0.00 | 0.00 | 0.00 | 0.00 | 0.00 | 0.00 | 0.00 | 0.03 | 0.00 | 0.00 |
| C | 0.00 | 0.00 | 0.00 | 0.00 | 0.00 | 0.00 | 0.00 | 0.00 | −1.82 | −0.69 | 3.08 | 0.00 | 0.00 |
| S | 0.00 | 0.00 | −0.45 | 0.00 | 0.00 | 0.17 | −0.27 | 0.00 | −0.61 | 0.03 | −1.05 | −0.27 | 0.10 |
| T | 0.00 | −0.18 | 0.03 | 0.00 | 0.00 | 0.83 | −0.57 | 0.00 | 0.00 | 0.00 | 0.00 | −0.16 | 0.05 |
| N | 0.00 | 0.03 | 0.00 | 0.00 | −0.30 | −0.61 | 0.00 | −0.30 | −1.82 | −0.48 | 0.00 | 0.00 | 0.00 |
| Q | −2.12 | −0.18 | 0.06 | 0.00 | 0.00 | 0.00 | 0.00 | 0.00 | 0.00 | 0.00 | 0.00 | 0.00 | 0.00 |
| Y | 0.00 | 0.03 | 0.00 | 0.00 | −0.30 | 0.00 | 0.00 | 0.00 | 6.24 | 0.48 | −0.30 | 0.00 | 0.00 |
| H | 0.00 | 0.03 | 0.00 | 0.00 | 0.00 | 0.00 | 0.00 | 0.00 | −1.75 | 0.41 | 0.00 | 0.00 | 0.00 |
| R | −0.30 | −0.23 | −0.57 | 0.00 | 0.00 | 0.00 | 0.00 | 0.00 | 0.00 | 0.00 | −0.91 | 0.00 | 0.00 |
| K | 0.00 | −0.19 | 0.00 | −0.30 | 0.00 | 0.00 | 0.00 | 0.00 | 0.00 | 0.00 | 0.00 | 0.00 | 0.00 |
| D | 0.00 | 0.00 | 0.00 | 0.00 | 2.36 | 0.00 | 0.03 | −0.14 | 0.00 | 0.00 | 0.00 | 0.00 | −0.75 |
| E | 0.00 | 0.58 | 0.00 | 1.68 | 0.06 | 0.00 | 0.00 | 0.00 | 0.00 | 0.00 | 0.00 | 0.00 | 0.00 |

CDR3

| | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| P | 0.00 | 1.96 | 0.00 | 0.00 | 0.00 | 0.00 | 0.00 | 0.00 | 0.00 | 0.00 | 0.00 | 0.00 | 0.00 | 0.00 | 0.00 |
| A | 0.00 | 0.03 | 0.00 | 0.00 | 0.00 | 0.00 | 0.00 | 0.00 | 0.00 | 0.00 | 0.00 | 0.00 | 0.00 | 0.00 | 0.00 |
| G | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| M | 0.71 | 0.00 | 0.00 | 0.00 | 0.00 | 0.00 | 0.00 | 0.00 | 0.00 | 0.00 | 0.00 | 0.00 | 0.00 | 0.00 | 0.00 |
| I | −1.32 | 0.00 | −1.15 | −0.62 | 0.00 | 0.00 | 0.00 | 0.00 | 0.00 | 0.00 | 0.00 | 0.00 | 0.00 | 0.00 | 0.00 |
| L | 2.64 | −0.91 | −0.53 | 2.52 | 0.00 | 0.00 | 0.00 | 0.00 | 0.00 | 0.00 | 0.00 | 0.00 | 0.00 | 0.00 | 0.00 |
| V | −0.54 | 0.00 | 0.03 | 0.06 | 0.00 | 0.00 | 0.00 | 0.00 | 0.00 | 0.00 | 0.00 | 0.00 | 0.00 | 0.00 | 0.00 |
| F | 0.00 | 0.00 | 3.74 | −1.37 | 0.00 | 0.00 | 0.00 | 0.00 | 0.00 | 0.00 | 0.00 | 0.00 | 0.00 | 0.00 | 0.00 |
| W | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| C | 0.00 | 0.00 | 0.03 | 0.16 | 0.00 | 0.00 | 0.00 | 0.00 | 0.00 | 0.00 | 0.00 | 0.00 | 0.00 | 0.00 | 0.00 |
| S | 0.00 | 0.06 | −1.84 | −0.75 | 0.00 | 0.00 | 0.00 | 0.00 | 0.00 | 0.00 | 0.00 | 0.00 | 0.00 | 0.00 | 0.00 |
| T | −0.57 | −0.27 | 0.00 | 0.00 | 0.00 | 0.00 | 0.00 | 0.00 | 0.00 | 0.00 | 0.00 | 0.00 | 0.00 | 0.00 | 0.00 |
| N | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| Q | 0.00 | −0.91 | 0.00 | 0.00 | 0.00 | 0.00 | 0.00 | 0.00 | 0.00 | 0.00 | 0.00 | 0.00 | 0.00 | 0.00 | 0.00 |
| Y | 0.00 | 0.00 | −0.29 | 0.00 | 0.00 | 0.00 | 0.00 | 0.00 | 0.00 | 0.00 | 0.00 | 0.00 | 0.00 | 0.00 | 0.00 |
| H | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| R | −0.61 | 0.03 | 0.00 | 0.00 | 0.00 | 0.00 | 0.00 | 0.00 | 0.00 | 0.00 | 0.00 | 0.00 | 0.00 | 0.00 | 0.00 |
| K | −0.30 | 0.00 | 0.00 | 0.00 | 0.00 | 0.00 | 0.00 | 0.00 | 0.00 | 0.00 | 0.00 | 0.00 | 0.00 | 0.00 | 0.00 |
| D | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| E | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |

CDR3

| | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|
| P | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| A | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| G | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| M | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| I | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| L | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| V | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| F | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| W | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| C | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| S | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| T | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| N | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| Q | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| Y | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| H | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| R | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| K | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| D | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| E | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |

Frame4

| | 1 | 2 | 3 | 4 | 5 | 6 | 7 | 8 | 9 | 10 | 11 | 12 | 13 |
|---|---|---|---|---|---|---|---|---|---|---|---|---|---|
| P | 0.00 | 0.00 | 0.00 | 0.00 | −0.11 | 0.00 | 0.00 | 0.21 | 0.00 | 0.00 | 0.00 | 0.06 | 0.00 |
| A | −0.30 | 0.00 | 0.00 | 0.00 | 0.00 | 0.03 | −0.05 | 0.00 | 0.06 | 0.06 | 0.06 | 0.00 | −0.54 |
| G | −0.48 | 0.00 | 0.10 | 1.12 | 0.00 | 0.14 | 0.00 | 0.00 | 0.00 | 0.00 | 0.00 | 0.00 | 0.10 |
| M | 0.00 | 0.00 | 0.00 | 0.00 | 0.00 | 0.00 | 0.00 | 0.00 | 0.00 | 0.00 | 0.00 | 0.00 | 0.00 |
| I | 0.00 | 0.00 | 0.00 | 0.00 | 0.00 | 0.00 | −0.14 | 0.00 | 0.00 | 0.00 | 0.00 | 0.00 | 0.06 |
| L | 0.00 | 0.03 | 0.10 | 0.00 | −0.61 | 0.00 | 0.00 | 0.20 | 0.00 | 0.00 | 0.00 | −0.54 | 0.00 |
| V | −0.51 | 0.00 | 0.00 | 0.03 | 0.00 | 0.00 | 0.03 | 0.00 | −0.06 | 0.00 | 0.18 | 0.00 | 0.00 |
| F | 0.00 | 0.83 | 0.00 | 0.00 | 0.00 | 0.00 | 0.00 | 0.00 | 0.00 | 0.00 | 0.00 | 0.00 | 0.00 |
| W | 0.00 | 0.00 | 0.80 | 0.00 | 0.00 | 0.00 | 0.00 | 0.00 | 0.00 | 0.00 | 0.00 | 0.00 | 0.00 |
| C | 0.00 | −0.08 | 0.13 | 0.00 | 0.00 | −0.24 | 0.00 | 0.00 | 0.00 | 0.00 | 0.00 | 0.00 | 0.13 |
| S | 0.00 | −0.61 | 0.03 | 0.03 | 0.00 | 0.06 | −1.08 | 0.00 | 0.00 | 0.10 | 0.00 | 0.38 | 0.40 |
| T | 0.00 | 0.00 | 0.00 | 0.00 | 0.00 | 0.00 | 1.21 | 0.00 | 0.00 | −0.19 | 0.00 | 0.00 | 0.00 |
| N | 0.22 | −0.57 | 0.00 | 0.00 | 0.00 | 0.00 | 0.00 | 0.00 | 0.00 | 0.00 | 0.00 | 0.00 | −0.30 |
| Q | 0.00 | 0.00 | 0.00 | 0.00 | 2.72 | 0.00 | 0.00 | 2.08 | 0.00 | 0.03 | 0.00 | 0.00 | 0.00 |
| Y | −0.21 | 0.79 | 0.00 | 0.00 | 0.00 | 0.00 | 0.00 | 0.00 | 0.00 | 0.00 | −0.24 | 0.10 | 0.00 |
| H | 0.00 | −0.38 | 0.00 | 0.00 | −2.14 | 0.00 | 0.00 | 0.10 | 0.00 | 0.00 | 0.00 | 0.00 | 0.00 |
| R | 0.00 | 0.00 | −1.15 | −0.91 | 0.25 | 0.00 | 0.03 | −2.38 | 0.00 | 0.00 | 0.00 | 0.00 | 0.16 |
| K | 0.00 | 0.00 | 0.00 | 0.00 | −0.24 | 0.00 | 0.00 | −0.21 | 0.00 | 0.00 | 0.00 | 0.00 | 0.00 |
| D | 0.67 | 0.00 | 0.00 | 0.00 | 0.00 | 0.00 | 0.00 | 0.00 | 0.00 | 0.00 | 0.00 | 0.00 | 0.00 |
| E | 0.60 | 0.00 | 0.00 | −0.27 | 0.13 | 0.00 | 0.00 | 0.00 | 0.00 | 0.00 | 0.00 | 0.00 | 0.00 |

TABLE 14
VHH variants ELISA and neutralization data. ELISA binding assay and pseudotyped virus
neutralization assay results for all VHH variants characterized in this study.

| sample_ID | ELISA A450 | Pseudovirus neutralization % inhibition | | | | | |
|---|---|---|---|---|---|---|---|
| VHH concentration | 50 nM | 500 nM | | 50 nM | | 5 nM | |
| SR1 | 0.375 | 32.96703297 | 14.03921569 | 17.10843373 | 13.77431907 | 15.42130366 | 12.67605634 |
| SR1_t2 | 0.263 | 20.25117739 | 13.41176471 | 8.43373494 | 7.237354086 | −1.907790143 | 10.17214397 |
| SR2 | 0.625 | 31.711146 | 19.52941176 | 4.738955823 | 4.124513619 | 1.430842607 | 4.225352113 |
| SR2_c3 | 0.455 | 67.81789639 | 59.37254902 | 16.30522088 | 16.42023346 | 0.635930048 | 4.851330203 |
| SR2_c4 | 1.650 | 49.45054945 | 38.03921569 | 12.77108434 | 11.28404669 | 14.30842607 | 2.660406886 |
| SR2_f3 | 0.006 | 6.017699115 | −0.622222222 | 5.008635579 | 2.670111972 | 2.586206897 | 0 |
| SR2_t5 | 0.333 | 61.2244898 | 52 | 14.0562249 | 18.13229572 | 2.702702703 | 13.92801252 |
| SR2_t9 | 0.799 | 23.54788069 | 20.78431373 | 13.41365462 | 9.883268482 | 12.40063593 | 12.05007825 |
| SR4 | 0.080 | 32.18210361 | 20 | 11.00401606 | 8.793774319 | 6.200317965 | 0.625978091 |
| SR4_c1 | 0.221 | 45.21193093 | 34.2745098 | 8.59437751 | 10.97276265 | −3.33863275 | 3.286384977 |
| SR4_f3 | 0.145 | 28.72841444 | 16.8627451 | 4.578313253 | 9.105058366 | 3.974562798 | 0 |
| SR4_t4 | 0.268 | 52.90423862 | 36.47058824 | 6.987951807 | 5.369649805 | 4.92845787 | −0.625978091 |
| SR4_t5 | 0.093 | 50.08849558 | 35.64444444 | 20.7253886 | 6.287683032 | −0.344827586 | 2.426343154 |
| SR4_t6 | 0.187 | 73.45132743 | 66.4 | 47.66839378 | 31.78294574 | 9.482758621 | 4.332755633 |
| SR4_t9 | 0.358 | 94.12389381 | 91.71555556 | 77.54749568 | 73.47114556 | 12.06896552 | 6.932409012 |
| SR4_t13 | 0.778 | 95.00784929 | 92.48627451 | 62.08835341 | 50.97276265 | 13.83147854 | 4.06885759 |
| SR6 | 0.042 | 31.86813187 | 19.37254902 | 14.53815261 | 14.24124514 | 13.19554849 | 9.546165884 |
| SR6_c3 | 0.709 | 86.27943485 | 75.52941176 | 66.74698795 | 58.44357977 | 27.82193959 | 24.25665102 |
| SR6_f3 | 0.153 | 21.41138733 | 19.33438986 | 15.54878049 | 11.90661479 | 11.48804934 | 8.647798742 |
| SR6_t4 | 0.705 | 84.18604651 | 77.81299525 | 64.78658537 | 59.53307393 | 20.1233616 | 16.98113208 |
| SR6_t5 | 1.038 | 65.03608661 | 61.33122029 | 39.48170732 | 32.45136187 | 12.10485736 | 8.805031447 |
| SR6_t6 | 0.708 | 69.20609463 | 62.12361331 | 32.62195122 | 28.87159533 | 4.240555127 | −1.257861635 |
| SR6_t9 | 0.657 | 90.26463512 | 82.2503962 | 63.87195122 | 64.82490272 | 24.59521974 | 11.16352201 |
| SR6_t12 | 0.505 | 86.35124298 | 80.34865293 | 60.97560976 | 57.3540856 | 16.5767155 | 3.93081761 |
| SR8 | 0.025 | 23.97754611 | 16.32329635 | 11.8902439 | 8.949416342 | 14.26368543 | 8.805031447 |
| SR8_c2 | 0.028 | 12.10906175 | 10.30110935 | 3.963414634 | 3.346303502 | 4.548959136 | 1.41509434 |
| SR8_t5 | 0.000 | 1.592920354 | −3.288888889 | −4.490500864 | −0.947459087 | −8.275862069 | −6.585788562 |
| SR12 | 0.935 | 8.4202085 | 4.595879556 | 2.134146341 | −0.077821012 | 1.00231303 | −0.471698113 |
| SR12_c3 | 0.547 | 51.72413793 | 36.76703645 | 13.87195122 | 9.883268482 | 0.077101002 | −2.51572327 |
| SR12_c5 | 0.462 | 12.91098637 | 5.229793978 | 10.67073171 | 3.968871595 | 8.712413261 | 3.616352201 |
| SR12_f3 | 0.465 | 14.03368083 | 8.557844691 | 11.58536585 | 5.992217899 | 12.72166538 | 3.93081761 |
| SR12_t6 | 0.784 | 44.66720128 | 35.340729 | 11.43292683 | 9.260700389 | 7.32459522 | 0.157232704 |
| SR12_t9 | 0.809 | 58.2300885 | 46.48888889 | 15.54404145 | 9.905254091 | −1.724137931 | −0.519930676 |
| VHH72 | 0.287 | 81.39534884 | 49.60380349 | 4.115853659 | 3.968871595 | 6.090979183 | 2.672955975 |

TABLE 15
High-throughput sequencing metadata. Number of sequences obtained
by high-throughput sequencing for indicated analyses.

| Initial selection for identifying | | cluster- | Unique |
|---|---|---|---|
| RBD and EGFP binders, number of | total | forming | cluster |
| sequences passing quality trimming | sequenced | sequences | sequences |
| input library | 107033 | 0 | 0 |
| RBD output after 3 rounds | 101647 | 83392 | 19223 |
| EGFP output after 3 rounds | 89754 | 71210 | 36379 |

| Affinity maturation of selected | pre-affinity | post-affinity |
|---|---|---|
| RBD binders, number of sequences | maturation | maturation |
| passing quality trimming | sequences | sequences |
| SR1 | 14596 | 6426 |
| SR2 | 255 | 48 |
| SR4 | 8624 | 85756 |
| SR6 | 330 | 3146 |
| SR8 | 10248 | 3377 |
| SR12 | 213 | 457 |

TABLE 16

| Cluster | Binding | Neutralizing |
|---|---|---|
| SR9 | non-binder | |
| SR6 | weak binder | Neutralizer |
| SR12 | strong binder | Neutralizer |
| SR8 | weak binder | Neutralizer |
| SR5 | non-binder | |
| SR14 | weak binder | |
| SR4 | weak binder | Neutralizer |
| SR10 | non-binder | |
| SR3 | non-binder | |
| SR1 | strong binder | Neutralizer |
| SR2 | strong binder | Neutralizer |
| SR11 | weak binder | |
| SR13 | weak binder | |
| SR7 | weak binder | |

### REFERENCES

1. Gray, A. C. et al. Animal-derived-antibody generation faces strict reform in accordance with European Union policy on animal use. Nat. Methods 17, 755-756 (2020).
2. Dübel, S., Stoevesandt, O., Taussig, M. J. & Hust, M. Generating recombinant antibodies to the complete human proteome. Trends Biotechnol. 28, 333-339 (2010).
3. Miersch, S. & Sidhu, S. S. Synthetic antibodies: Concepts, potential and practical considerations. Methods 57, 486-498 (2012).
4. Gray, A. et al. Animal-free alternatives and the antibody iceberg. Nat. Biotechnol. 38, 1234-1239 (2020).
5. Bradbury, A. R. M., Sidhu, S., Dübel, S. & McCafferty, J. Beyond natural antibodies: The power of in vitro display technologies. Nat. Biotechnol. 29, 245-254 (2011).
6. McMahon, C. et al. Yeast surface display platform for rapid discovery of conformationally selective nanobodies. Nat. Struct. Mol. Biol. 25, 289-296 (2018).
7. Moutel, S. et al. NaLi—H1: A universal synthetic library of humanized nanobodies providing highly functional antibodies and intrabodies. Elife 5, 1-31 (2016).
8. Zimmermann, I. et al. Synthetic single domain antibodies for the conformational trapping of membrane proteins. Elife 7, 1-32 (2018).
9. Muyldermans, S. Nanobodies: Natural single-domain antibodies. Annu. Rev. Biochem. 82, 775-797 (2013).
10. Turner, K. B., Zabetakis, D., Goldman, E. R. & Anderson, G. P. Enhanced stabilization of a stable single domain antibody for SEB toxin by random mutagenesis and stringent selection. Protein Eng. Des. Sel. 27, 89-95 (2014).
11. Huo, J. et al. Neutralizing nanobodies bind SARS-CoV-2 spike RBD and block interaction with ACE2. Nat. Struct. Mol. Biol. (2020). doi:10.1038/s41594-020-0469-6
12. Boder, E. T. & Wittrup, K. D. Yeast surface display for screening combinatorial polypeptide libraries. Nat. Biotechnol. 15, 553-557 (1997).
13. Hanes, J. & Plückthun, A. In vitro selection and evolution of functional proteins by using ribosome display. Proc. Natl. Acad. Sci. U.S.A 94, 4937-4942 (1997).
14. Hanes, J., Schaffitzel, C., Knappik, A. & Püickthun, A. Picomolar affinity antibodies from a fully synthetic naive library selected and evolved by ribosome display. Nat. Biotechnol. 18, 1287-1292 (2000).
15. He, M. & Taussig, M. J. Ribosome display: Cell-free protein display technology. Briefings Funct. Genomics Proteomics 1, 204-212 (2002).
16. Kirchhofer, A. et al. Modulation of protein properties in living cells using nanobodies. Nat. Struct. Mol. Biol. 17, 133-139 (2010).
17. Zhou, P. et al. A pneumonia outbreak associated with a new coronavirus of probable bat origin. Nature 579, 270-273 (2020).
18. Egloff, P. et al. Engineered peptide barcodes for in-depth analyses of binding protein libraries. Nat. Methods 16, 421-428 (2019).
19. Wrapp, D. et al. Structural Basis for Potent Neutralization of Betacoronaviruses by Single-Domain Camelid Antibodies. Cell 1004-1015 (2020). doi:10.1016/j.cell.2020.04.031
20. Rogers, T. F. et al. Isolation of potent SARS-CoV-2 neutralizing antibodies and protection from disease in a small animal model. Science 7520, eabc7520 (2020).
21. Hansen, J. et al. Studies in humanized mice and convalescent humans yield a SARS-CoV-2 antibody cocktail. Science 0827, eabd0827 (2020).
22. Scheid, J. F. et al. B cell genomics behind cross-neutralization of SARS-CoV-2 variants and SARS-CoV. Cell 184, 3205-3221.e24 (2021).
23. Li, W. et al. High Potency of a Bivalent Human VH Domain in SARS-CoV-2 Animal Models. Cell 183, 429-441.e16 (2020).
24. Li, Q. et al. SARS-CoV-2 501Y.V2 variants lack higher infectivity but do have immune escape. Cell (2021). doi:10.1016/j.cell.2021.02.042
25. Hanke, L. et al. An alpaca nanobody neutralizes SARS-CoV-2 by blocking receptor interaction. Nat. Commun. 11, 1-9 (2020).
26. Xiang, Y. et al. Versatile and multivalent nanobodies efficiently neutralize SARS-CoV-2. Science 1484, eabe4747 (2020).
27. Moore, M. J. et al. Retroviruses Pseudotyped with the Severe Acute Respiratory Syndrome Coronavirus Spike Protein Efficiently Infect Cells Expressing Angiotensin-Converting Enzyme 2.J. Virol. 78, 10628-10635 (2004).
28. Gentili, M. et al. Transmission of innate immune signaling by packaging of cGAMP in viral particles. Science 349, 1232-1236 (2015).
29. Raab, M. et al. ESCRT III repairs nuclear envelope ruptures during cell migration to limit DNA damage and cell death. Science 352, 359-362 (2016).
30. Henikoff, S. & Henikoff, J. G. Amino acid substitution matrices from protein blocks. Proc. Natl. Acad. Sci. U.S.A 89, 10915-10919 (1992).
31. Cohen, J. (2020). Antibodies may curb pandemic before vaccines. Science 369, 752-753.
32. Kirchhofer, A., Helma, J., Schmidthals, K., Frauer, C., Cui, S., Karcher, A., Pellis, M., Muyldermans, S., Casas-Delucchi, C. S., Cardoso, M. C., et al. (2010). Modulation of protein properties in living cells using nanobodies. Nat. Struct. Mol. Biol. 17, 133-139.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features hereinbefore set forth.

1. An antibody or antigen binding fragment comprising one or more complementarity-determining regions (CDRs) selected or derived from any cluster or CDRs in any of Tables 1-9, preferably,
wherein the CDRs are selected or derived from the SR1, SR2, SR4, SR6, SR8, SR12, SR15, SR18, SR25, SR30 or SR38 cluster families; or
wherein the antibody or antigen binding fragment comprises CDRs from SR6v15, SR6v7, SR38, SR6c3, SR4t13, or SR2c3.

2-3. (canceled)

4. The antibody or antigen binding fragment of claim 1, wherein the antibody or antigen binding fragment is a heavy chain antibody or variable domain of the heavy chain (VHH), preferably,
wherein the heavy chain antibody or variable domain of the heavy chain (VHH) is SR38 and binds to a N501Y SARS-CoV-2 variant; or
wherein the heavy chain antibody or variable domain of the heavy chain (VHH) is SR6v15; or
wherein the heavy chain antibody or variable domain of the heavy chain (VHH) is a dimer of SR6v15; or
wherein the heavy chain antibody or variable domain of the heavy chain (VHH) is SR6v7.

5-8. (canceled)

9. The antibody or antigen binding fragment of claim 4, wherein the heavy chain antibody or VHH are derived from camelid heavy chain antibodies, preferably, wherein one or more framework residues in camelid antibodies are humanized, more preferably, wherein the humanized residues are located in one or more positions selected from the group consisting of frame 2 position 4, frame 2 position 11, frame 2 position 12, frame 2 position 14, and frame 4 position 8.

10-11. (canceled)

12. The antibody or antigen binding fragment of claim 1, wherein the antibody or antigen binding fragment is modified to alter binding affinity, stability, in vivo half-life, neutralizing activity and/or dimerization, preferably, wherein the antibody or antigen binding fragment is a fusion protein, more preferably, wherein the antibody or antigen binding fragment is fused to another antibody or antibody fragment, Fc domain, antigen binding domain, glutathione S-transferase (GST), and/or serum albumin.

13-14. (canceled)

15. A method of treating SARS-CoV-2 infection comprising administering to a subject in need thereof the antibody or antigen binding fragment of claim 1, preferably,
wherein the subject is infected with a SARS-CoV-2 variant, more preferably,
wherein SR38 is administered to the subject, more preferably, wherein the subject is infected with a SARS-CoV-2 variant containing the N501Y mutation; or
wherein SR6v15 is administered to the subject; or
wherein a dimer of SR6v15 is administered to the subject.

16-20. (canceled)

21. A method of detecting SARS-CoV-2 comprising contacting a biological sample obtained from a subject with the antibody or antigen binding fragment of claim 1, preferably,
wherein the antibody is SR38 and a variant containing the N501Y mutation is detected; or
wherein the antibody is SR38 and a variant containing the E484K mutation is detected; or
wherein the antibody is SR6v15.

22-24. (canceled)

25. A method of generating a VHH library comprising a VHH template with a randomized CDR1, CDR2 and CDR3 comprising:
a. providing a VHH template;
b. providing a first set of primers capable of amplifying the VHH template from a first CDR sequence to the end of the template, wherein the set of primers comprise:
i. a primer comprising a 5′ randomized sequence corresponding to all or part of the first CDR sequence and a 3′ sequence capable of hybridizing to a non-randomized sequence; and
ii. a hairpin primer capable of hybridizing to one end of the template;
c. providing a second set of primers capable of amplifying the VHH template from the sequence directly adjacent to where the first primer set amplified from to the other end of the template, wherein the set of primers comprise:
i. a primer capable of hybridizing to the sequence directly adjacent to where the first primer set amplified from, optionally, wherein the primer starts within the first CDR sequence and comprises a 5′ randomized sequence corresponding to the remaining first CDR sequence and a 3′ sequence capable of hybridizing to a non-randomized sequence; and
ii. a hairpin primer capable of hybridizing to the other end of the template;
d. PCR amplifying the VHH template with the first and second sets of primers to generate two single-end blocked PCR products corresponding to the entire VHH template;
e. ligating the two PCR products;
f. repeating steps (a) to (e) for the second CDR sequence, wherein the randomized VHH ligation product obtained in step (e) is used as the template, whereby a VHH template randomized for two CDRs is obtained; and
g. repeating steps (a) to (e) for the third CDR sequence, wherein the randomized VHH ligation product obtained in step (f) is used as the template, whereby a VHH template randomized for all three CDRs is obtained.

26. The method of claim 25, wherein the primer sequences are 5′ NNB randomized, where N is a mixture of A, T, G, C bases, and B is a mixture of G, C, T bases; and/or wherein the primer sequences are 5′ randomized using NNN tri-nucleotide sequence, where N is a mixture of A, T, C, G nucleotides.

27. (canceled)

28. The method of claim 25, wherein step (d) is performed using a DNA polymerase without strand displacement activity or with weak strand displacement activity; and/or
wherein step (d) is performed using an elongation temperature of 65° C.

29. (canceled)

30. The method of claim 25, wherein CDR2 is randomized first; and/or
wherein CDR1 is randomized second; and/or
wherein CDR3 is randomized last and/or
wherein CDR2 encodes for 4 or 5 amino acids; and/or
wherein CDR1 encodes for 4 to 8 amino acids; and/or
wherein CDR3 encodes for 4 to 30 amino acids.

31-35. (canceled)

36. The method of claim 25, wherein the VHH templates comprise a promoter sequence upstream of the VHH template, optionally, wherein the promoter is a T7 promoter.

37. (canceled)

38. The method of claim 25, wherein the VHH templates comprise an epitope tag sequence downstream of and in frame with the VHH template, optionally, wherein the epitope tag comprises one or more myc tags.

39. (canceled)

40. The method of claim 38, further comprising displaying the CDR1 and/or CDR2 randomized library of step (e) or (f) with ribosome display; enriching library members using the epitope tag; and using the enriched DNAs for input in step (g).

41. The method of claim 25, wherein the VHH templates do not include a stop codon.

42. A method of identifying CDRs for generating an antibody or binding fragment of an antibody specific to an antigen of interest comprising:
a. providing a linear DNA library, wherein each sequence in the library encodes for an antibody framework comprising three CDRs and operably linked to a 5′ promoter sequence, and wherein at least one CDR is randomized;
b. performing ribosome display on the linear DNA library, whereby mRNAs transcribed from the linear DNA library are translated to an antibody protein that is tethered to the ribosome ribonucleoprotein complex;
c. binding the ribonucleoprotein complexes to an immobilized antigen of interest;
d. performing reverse transcription PCR (RT-PCR) on mRNA extracted from ribonucleoprotein complexes bound to the immobilized antigen, whereby cDNA is generated;
e. optionally, repeating steps (b) to (d) using the cDNA from the bound ribonucleoprotein complexes as the linear DNA input;
f. sequencing the cDNAs to obtain antibody sequences; and
g. clustering the antibody sequences based on similarity of their CDRs to identify distinct antibody clusters containing CDRs specific to the antigen of interest.

43. The method of claim 42, wherein all three CDRs are randomized.

44. The method of claim 42, wherein the CDRs are encoded by DNA oligos with 5′ NNB or NNN randomized sequences, where N is a mixture of A, T, G, C bases, and B is a mixture of G, C, T bases; and/or
wherein step (c) is performed in solutions containing Mg²⁺ ions at concentrations of 5 mM or less; and/or
wherein step (d) is performed using a mixture of two DNA polymerases in the PCR reaction, wherein one type is a DNA polymerase without strand displacement activity or with weak strand displacement activity and the other type is a DNA polymerase with strong strand displacement activity; and/or
wherein steps (b) to (d) are performed for three rounds.

45-47. (canceled)

48. The method of claim 42, further comprising identifying amino acid substitutions that will increase antibody binding and/or viral neutralization activity, said method comprising:
h. introducing random mutations across the full length of one or more identified antibody frameworks using error prone PCR to obtain a mutated linear DNA library;
i. repeating steps (b) to (d) for 1 to 3 rounds using the linear DNA library obtained in step (h) as the linear DNA library;
j. sequencing the linear DNA library in (h) and the cDNA obtained in (i);
k. calculating the percentage of each proteinogenic amino acid found at each antibody framework amino acid position among all sequenced antibody frameworks obtained from (h) and (i);
l. identifying amino acids at each position with increased percentage in (i) as compared to in sequences from (h); and
m. replacing the amino acids at said positions in the antibody framework with the identified amino acids.

49. The method of claim 42, wherein step (c) is performed using a binding time of less than 1 minute; and/or
wherein CDRs are selected from one or more of the clusters having the largest number of members; and/or
wherein the antibody frameworks are heavy chain antibody variable domains (VHHs), optionally, wherein the VHHs are camelid VHHs.

50-52. (canceled)

53. The method of claim 42, wherein the linear DNA library in step (a) is obtained according to a method comprising:
a. providing a VHH template;
b. providing a first set of primers capable of amplifying the VHH template from a first CDR sequence to the end of the template, wherein the set of primers comprise:
i. a primer comprising a 5′ randomized sequence corresponding to all or part of the first CDR sequence and a 3′ sequence capable of hybridizing to a non-randomized sequence; and
ii. a hairpin primer capable of hybridizing to one end of the template;
c. providing a second set of primers capable of amplifying the VHH template from the sequence directly adjacent to where the first primer set amplified from to the other end of the template, wherein the set of primers comprise:
i. a primer capable of hybridizing to the sequence directly adjacent to where the first primer set amplified from, optionally, wherein the primer starts within the first CDR sequence and comprises a 5′ randomized sequence corresponding to the remaining first CDR sequence and a 3′ sequence capable of hybridizing to a non-randomized sequence; and
ii. a hairpin primer capable of hybridizing to the other end of the template;
d. PCR amplifying the VHH template with the first and second sets of primers to generate two single-end blocked PCR products corresponding to the entire VHH template;
e. ligating the two PCR products;
f. repeating steps (a) to (e) for the second CDR sequence, wherein the randomized VHH ligation product obtained in step (e) is used as the template, whereby a VHH template randomized for two CDRs is obtained; and
g. repeating steps (a) to (e) for the third CDR sequence, wherein the randomized VHH ligation product obtained in step (f) is used as the template, whereby a VHH template randomized for all three CDRs is obtained.

54. The method of claim 42, further comprising validating at least one member of a cluster or VHH sequence with amino acid substitutions by expressing the antibody framework and determining binding to the antigen of interest; and/or
wherein the antigen of interest is associated with a viral pathogen and the antibody framework is tested for neutralizing activity; and/or
further comprising transferring one or more of the CDRs to a different antibody framework; and/or
further comprising synthesizing one or more sequences from each antibody cluster for cloning of the antibody genes and testing the antibody proteins.

55-57. (canceled)
